(19) United States
(12) Patent Application Publication
(10) Pub. No.: US 2023/0148214 A1
(43) Pub. Date: May 11, 2023

(54) INDAZOLES AND AZAINDAZOLES AS LRRK2 INHIBITORS

(71) Applicant: ESCAPE Bio, Inc., South San Francisco, CA (US)

(72) Inventors: Albert W. Garofalo, South San Francisco, CA (US); Jacob Bradley Schwarz, San Ramon, CA (US); Fabio Maria Sabbatini, Verona (IT); Silvia Bernardi, Verona (IT); Marco Migliore, Verona (IT); Federica Budassi, Urbino (IT); Claudia Beato, Verona (IT)

(21) Appl. No.: 17/908,977

(22) PCT Filed: Mar. 5, 2021
(86) PCT No.: PCT/US2021/021054

§ 371 Date: Sep. 2, 2022

Related U.S. Application Data
(60) Provisional application No. 62/986,474, filed on Mar. 6, 2020.

(51) Int. Cl. C07D 491/052; C07D 231/54; C07D 401/12; C07D 487/04; C07D 471/04; C07D 519/00

(52) U.S. Cl. CPC C07D 491/052; C07D 231/54; C07D 401/12; C07D 487/04; C07D 471/04; C07D 519/00

(57) ABSTRACT

The present invention is directed to indazole and azaindazole compounds which are inhibitors of LRRK2 and are useful in the treatment of CNS disorders.

### FIELD OF THE INVENTION

The present invention is directed to indazole and azaindazole compounds which are inhibitors of LRRK2 and are useful in the treatment of CNS disorders.

### BACKGROUND OF THE INVENTION

Parkinson's disease (“PD”) is the most common form of parkinsonism, a movement disorder, and the second most common, age-related neurodegenerative disease estimated to affect 1-2% of the population over age 65. PD is characterized by tremor, rigidity, postural instability, impaired speech, and bradykinesia. It is a chronic, progressive disease with increasing disability and diminished quality of life. In addition to PD, parkinsonism is exhibited in a range of conditions such as progressive supranuclear palsy, corticobasal degeneration, multiple system atrophy, and dementia with Lewy bodies.

Current therapeutic strategies for PD are primarily palliative and focus on reducing the severity of symptoms using supplemental dopaminergic medications. At present, there is no disease-modifying therapy that addresses the underlying neuropathological cause of the disease, thus constituting a significant unmet medical need.

It has long been known that family members of PD patients have an increased risk of developing the disease compared to the general population. Leucine-rich repeat kinase 2 (“LRRK2,” also known as dardarin) is a 286 kDa multi-domain protein that has been linked to PD by genome-wide association studies. LRRK2 expression in the brain is highest in areas impacted by PD (Eur. J. Neurosci. 2006, 23(3):659) and LRRK2 has been found to localize in Lewy Bodies, which are intracellular protein aggregates considered to be a hallmark of the disease. Patients with point mutations in LRRK2 present disease that is indistinguishable from idiopathic patients. While more than 20 LRRK2 mutations have been associated with autosomal-dominantly inherited parkinsonism, the G2019S mutation located within the kinase domain of LRRK2 is by far the most common. This particular mutation is found in >85% of LRRK2-linked PD patients. It has been shown that the G2019S mutation in LRRK2 leads to an enhancement in LRRK2 kinase activity and inhibition of this activity is a therapeutic target for the treatment of PD.

In addition to PD, LRRK2 has been linked to other diseases such as cancer, leprosy, and Crohn's disease (Sci. Signal., 2012, 5(207), pe2). As there are presently limited therapeutic options for treating PD and other disorders associated with aberrant LRRK2 kinase activity, there remains a need for developing LRRK2 inhibitors.

### SUMMARY OF THE INVENTION

The present invention is directed to a compound of Formula I.

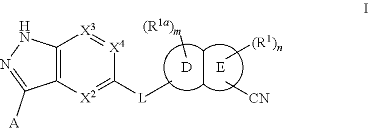

or a pharmaceutically acceptable salt thereof, wherein constituent members are defined herein.

The present invention is further directed to a pharmaceutical composition comprising a compound of Formula I, or a pharmaceutically acceptable salt thereof, and at least one pharmaceutically acceptable carrier.

The present invention is further directed to a method of inhibiting LRRK2 activity, comprising contacting a compound of Formula I, or a pharmaceutically acceptable salt thereof, with LRRK2.

The present invention is further directed to a method of treating a disease or disorder associated with elevated expression or activity of LRRK2, or a functional variant thereof, said method comprising administering to a patient in need thereof a therapeutically effective amount of a compound of Formula I, or a pharmaceutically acceptable salt thereof.

The present invention is further directed to a method for treating a neurodegenerative disease in a patient comprising administering to the patient a therapeutically effective amount of the compound of Formula I, or a pharmaceutically acceptable salt thereof.

The present invention is further directed to a compound of the invention, or a pharmaceutically acceptable salt thereof, for use in the treatment of a disease associated with elevated expression or activity of LRRK2, or a functional variant thereof. The present invention is further directed to use of a compound of the invention, or a pharmaceutically acceptable salt thereof, in the manufacture of a medicament for use in therapy.

### DETAILED DESCRIPTION

The present invention is directed to an inhibitor of LRRK2 which is a compound of Formula I:

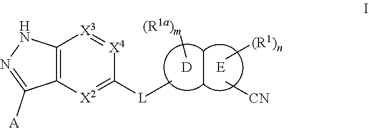

or a pharmaceutically acceptable salt thereof, wherein:

A is selected from Cy¹, Cy¹-C₁₋₄ alkyl-, Cy¹-C₂₋₄ alkenyl-, halo, C₁₋₆ alkyl, C₂₋₆ alkenyl, C₂₋₆ alkynyl, C₁₋₆ haloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, C(═NR^{e})NR^{c}R^{d}, NR^{c}C(═NR^{e})NR^{c}R^{d}, NR^{c}R^{d}, NR^{c}C(O)R^{b}, NR^{c}C(O)OR^{a}, NR^{c}C(O)NR^{c}R^{d}, NR^{c}C(S)NR^{c}R^{d}, NR^{c}S(O)R^{b}, NR^{c}S(O)₂R^{b}, NR^{c}S(O)₂NR^{c}R^{d}, S(O)R^{b}, S(O)NR^{e}R^{d}, S(O)₂R^{b}, and S(O)₂NR^{e}R^{d}; wherein said C₁₋₆ alkyl, C₂₋₆ alkenyl, C₂₋₆ alkynyl, and C₁₋₆ haloalkyl of A are each optionally substituted with 1, 2, 3, 4, or 5 substituents independently selected from halo, C₁₋₆ alkyl, C₂₋₆ alkenyl, C₂₋₆ alkynyl, C₁₋₆ haloalkyl, C₃₋₇ cycloalkyl, CN, NO₂, OR^{a}, SR^{a}, C(O)R^{b}, C(O)NR^{c}R^{d}, C(O)OR^{a}, OC(O)R^{b}, C(O)NR^{c}R^{d}, C(═NR^{e})NR^{c}R^{d}, NR^{c}C(═NR^{e})NR^{c}R^{d}, NR^{c}R^{d}, NR^{c}C(O)R^{b}, NR^{c}C(O)OR^{a}, NR^{c}C(O)NR^{c}R^{d}, NR^{c}S(O)R^{b}, NR^{c}S(O)₂R^{b}, NR^{c}S(O)₂NR^{c}R^{d}, S(O)R^{b}, S(O)NR^{c}R^{d}, S(O)₂R^{b}, and S(O)₂NR^{c}R^{d};

L is O, S, or NR^{N};

R^{N} is H or C₁₋₄ alkyl;

X² is N or CR²;

X³ is N or CR³;

X⁴ is N or CR⁴; wherein not more than two of X², X³, and X⁴ are simultaneously N;

Ring D is a C₄₋₇ cycloalkyl group or a 4-7 membered heterocycloalkyl group, each of which is fused with Ring E;

Ring E is phenyl or a 5- to 6-membered heteroaryl group, fused with Ring D;

Cy¹ is selected from C₆₋₁₀ aryl, C₃₋₁₀ cycloalkyl, 5-14 membered heteroaryl, and 4-14 membered heterocycloalkyl, each optionally substituted by 1, 2, 3, 4, or 5 substituents independently selected from halo, C₁₋₆ alkyl, C₂₋₆ alkenyl, C₂₋₆ alkynyl, C₁₋₆ haloalkyl, C₆₋₁₀ aryl, C₃₋₁₀ cycloalkyl, 5-14 membered heteroaryl, 4-14 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, C(O)N(R^{c})OR^{a}, NR^{c}R^{d}, NR^{c}C(O)R^{b}, NR^{c}C(O)OR^{a}, NR^{c}C(O)NR^{c}R^{d}, C(═NR^{e})R^{b}, C(═NR^{e})NR^{c}R^{d}, NR^{c}C(═NR^{e})NR^{c}R^{d}, NR^{c}S(O)R^{b}, NR^{c}S(O)₂R^{b}, NR^{c}S(O)₂NR^{c}R^{d}, S(O)R^{b}, S(O)NR^{c}R^{d}, S(O)₂R^{b}, and S(O)₂NR^{c}R^{d}, wherein said substituents C₁₋₆ alkyl, C₂₋₆ alkenyl, C₂₋₆ alkynyl, C₁₋₆ haloalkyl, C₆₋₁₀ aryl, C₃₋₁₀ cycloalkyl, 5-14 membered heteroaryl, 4-14 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl are each optionally substituted by 1, 2, or 3 further substituents independently selected from halo, C₁₋₆ alkyl, C₂₋₆ alkenyl, C₂₋₆ alkynyl, C₁₋₆ haloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)OR^{a}, NR^{c}C(O)NR^{c}R^{d}, C(═NR^{e})R^{b}, C(═NR^{e})NR^{c}R^{d}, NR^{c}C(═NR^{e})NR^{c}R^{d}, NR^{c}S(O)R^{b}, NR^{c}S(O)₂R^{b}, NR^{c}S(O)₂NR^{c}R^{d}, S(O)R^{b}, S(O)NR^{c}R^{d}, S(O)₂R^{b}, and S(O)₂NR^{c}R^{d};

each R¹ and R^{1a} is independently selected from H, D, halo, C₁₋₆ alkyl, C₂₋₆ alkenyl, C₂-6 alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}C(S)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}; wherein said C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R¹ are each optionally substituted with 1, 2, 3, 4, or 5 substituents independently selected from halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};

or two R¹ groups together with the atoms to which they are attached form a C₃₋₇ cycloalkyl group which is optionally substituted with 1, 2, 3, 4, or 5 substituents independently selected from halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{a1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{a1}, S(O)₂R^{b1}, and S(O)₂NR^{c1}R^{d1};

or two R^{1a} groups together with the atoms to which they are attached form a C₃₋₇ cycloalkyl group which is optionally substituted with 1, 2, 3, 4, or 5 substituents independently selected from halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};

R² and R⁴ are each independently selected from H, D, halo, C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a}2, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2}, wherein said C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R² and R⁴ are each optionally substituted with 1, 2, 3, 4, or 5 substituents independently selected from halo, C₁₋₆ alkyl, C₂₋₆ alkenyl, C₂₋₆ alkynyl, C₁₋₆ haloalkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2};

R³ is selected from H, D, halo, C₁₋₆ alkyl, C₂₋₆ alkenyl, C₂₋₆ alkynyl, C₁₋₆ haloalkyl, C₃-4 cycloalkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, C(═NR^{e3})R^{b3}, C(═NR^{e3})NR^{c3}R^{d3}, NR^{c3}C(═NR^{e3})NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3}, wherein said C₁₋₆ alkyl, C₂₋₆ alkenyl, C₂₋₆ alkynyl, C₁₋₆ haloalkyl, and C₃₋₄ cycloalkyl of R³ are each optionally substituted with 1, 2, or 3 substituents independently selected from halo, C₁₋₄ alkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, C(═NR^{e3})R^{b3}, C(═NR^{e3})NR^{c3}R^{d3}, NR^{c3}C(═NR^{e3})NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3};

each R^{a}, R^{b}, R^{c}, R^{d}, R^{a1}, R^{b1}, R^{c1}, R^{d1}, R^{a2}, R^{b2}, R^{c2}, and R^{d2} is independently selected from H, D, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl, wherein said C₁₋₆ alkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{a}, R^{b}, R^{c}, R^{d}, R^{a1}, R^{b1}, R^{c1}, R^{d1}, R^{a2}, R^{b2}, R^{c2}, and R^{d2} is optionally substituted with 1, 2, 3, 4, or 5 substituents independently selected from halo, C₁₋₄ alkyl, C₁₋₄ haloalkyl, C₁₋₆ haloalkyl, C₂₋₆ alkenyl, C₂₋₆ alkynyl, CN, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)NR^{c3}R^{d3}, NR^{c3}C(O)OR^{a3}, C(═NR^{e3})NR^{c3}R^{d3}, NR^{c3}C(═NR^{e3})NR^{c3}R^{d3}, S(O)R^{b3}, S(O)NR^{c3}R^{d3}, S(O)₂R^{b3}, NR^{c3}S(O)₂R^{b3}, NR^{c3}S(O)₂NR^{c3}R^{d3}, and S(O)₂NR^{c3}R^{d3};

each R^{a3}, R^{b3}, R^{c3}, and R^{d3} is independently selected from H, D, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-6 membered heteroaryl, and 4-7 membered heterocycloalkyl, wherein said C₁₋₆ alkyl, C₁₋₆ haloalkyl, C₂₋₆ alkenyl, C₂₋₆ alkynyl, C₆₋₁₀ aryl, C₃₋₇ cycloalkyl, 5-6 membered heteroaryl, and 4-7 membered heterocycloalkyl are each optionally substituted with 1, 2, or 3 substituents independently selected from OH, CN, amino, halo, C₁₋₆ alkyl, C₁₋₆ alkoxy, C₁₋₆ haloalkyl, and C₁₋₆ haloalkoxy;

each R^{e}, R^{e1}, R^{e2}, and R^{e3} is independently selected from H, D, C₁₋₄ alkyl, and CN;

n is 0, 1, 2, or 3; and

m is 0, 1 or 2.

Also provided herein is a compound of Formula I:

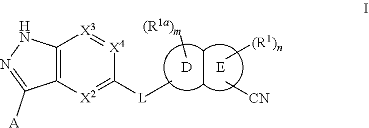

or a pharmaceutically acceptable salt thereof, wherein:

A is selected from Cy¹, Cy¹-C₁₋₄ alkyl-, halo, C₁₋₆ alkyl, C₂₋₆ alkenyl, C₂₋₆ alkynyl, C₁₋₆ haloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, C(═NR^{e})NR^{c}R^{d}, NR^{c}C(═NR^{e})NR^{c}R^{d}, NR^{c}R^{d}, NR^{c}C(O)R^{b}, NR^{c}C(O)OR^{a}, NR^{c}C(O)NR^{c}R^{d}, NR^{c}S(O)R^{b}, NR^{c}S(O)₂R^{b}, NR^{c}S(O)₂NR^{c}R^{d}, S(O)R^{b}, S(O)NR^{c}R^{d}, S(O)₂R^{b}, and S(O)₂NR^{c}R^{d}; wherein said C₁₋₆ alkyl, C₂₋₆ alkenyl, C₂₋₆ alkynyl, and C₁₋₆ haloalkyl of A are each optionally substituted with 1, 2, 3, 4, or 5 substituents independently selected from halo, C₁₋₆ alkyl, C₂₋₆ alkenyl, C₂₋₆ alkynyl, C₁₋₆ haloalkyl, C₃₋₇ cycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, C(═NR^{e})NR^{c}R^{d}, NR^{c}C(═NR^{e})NR^{c}R^{d}, NR^{c}R^{d}, NR^{c}C(O)R^{b}, NR^{c}C(O)OR^{a}, NR^{c}C(O)NR^{c}R^{d}, NR^{c}S(O)R^{b}, NR^{c}S(O)₂R^{b}, NR^{c}S(O)₂NR^{c}R^{d}, S(O)R^{b}, S(O)NR^{c}R^{d}, S(O)₂R^{b}, and S(O)₂NR^{c}R^{d};

L is O, S, or NR^{N};

R^{N} is H or C₁₋₄ alkyl;

X² is N or CR²;

X³ is N or CR³;

X⁴ is N or CR⁴; wherein not more than two of X², X³, and X⁴ are simultaneously N;

Ring D is a C₄₋₇ cycloalkyl group fused with Ring E;

Ring E is phenyl or a 5- to 6-membered heteroaryl group, fused with Ring D;

Cy¹ is selected from C₆₋₁₀ aryl, C₃₋₁₀ cycloalkyl, 5-14 membered heteroaryl, and 4-14 membered heterocycloalkyl, each optionally substituted by 1, 2, 3, 4, or 5 substituents independently selected from halo, C₁₋₆ alkyl, C₂₋₆ alkenyl, C₂₋₆ alkynyl, C₁₋₆ haloalkyl, C₆₋₁₀ aryl, C₃₋₁₀ cycloalkyl, 5-14 membered heteroaryl, 4-14 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a}, SR^{a}, C(O)R^{b}, C(O)NR^{e}R^{d}, C(O)OR^{a}, OC(O)R^{b}, OC(O)NR^{c}R^{d}, NR^{c}R^{d}, NR^{c}C(O)R^{b}, NR^{c}C(O)OR^{a}, NR^{c}C(O)NR^{c}R^{d}, C(═NR^{e})R^{b}, C(═NR^{e})NR^{c}R^{d}, NR^{c}C(═NR^{e})NR^{c}R^{d}, NR^{c}S(O)R^{b}, NR^{c}S(O)₂R^{b}, NR^{c}S(O)₂NR^{c}R^{d}, S(O)R^{b}, S(O)NR^{e}R^{d}, S(O)₂R^{b}, and S(O)₂NR^{e}R^{d}, wherein said substituents C₁₋₆ alkyl, C₂₋₆ alkenyl, C₂₋₆ alkynyl, C₁₋₆ haloalkyl, C₆₋₁₀ aryl, C₃₋₁₀ cycloalkyl, 5-14 membered heteroaryl, 4-14 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl are each optionally substituted by 1, 2, or 3 further substituents independently selected from halo, C₁₋₆ alkyl, C₂₋₆ alkenyl, C₂₋₆ alkynyl, C₁₋₆ haloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)OR^{a}, NR^{c}C(O)NR^{c}R^{d}, C(═NR^{e})R^{b}, C(═NR^{e})NR^{c}R^{d}, NR^{c}C(═NR^{e})NR^{c}R^{d}, NR^{c}S(O)R^{b}, NR^{c}S(O)₂R^{b}, NR^{c}S(O)₂NR^{c}R^{d}, S(O)R^{b}, S(O)NR^{c}R^{d}, S(O)₂R^{b}, and S(O)₂NR^{c}R^{d};

each R¹ and R^{1a} is independently selected from H, halo, C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}; wherein said C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R¹ are each optionally substituted with 1, 2, 3, 4, or 5 substituents independently selected from halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};

or two R¹ groups together with the atoms to which they are attached form a C₅₋₇ cycloalkyl group which is optionally substituted with 1, 2, 3, 4, or 5 substituents independently selected from halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};

R² and R⁴ are each independently selected from H, halo, C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a}2, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2}, wherein said C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R² and R⁴ are each optionally substituted with 1, 2, 3, 4, or 5 substituents independently selected from halo, C₁₋₆ alkyl, C₂₋₆ alkenyl, C₂₋₆ alkynyl, C₁₋₆ haloalkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2};

R³ is selected from H, halo, C₁₋₆ alkyl, C₂₋₆ alkenyl, C₂₋₆ alkynyl, C₁₋₆ haloalkyl, C₃₋₄ cycloalkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, C(═NR^{e3})R^{b3}, C(═NR^{e3})NR^{c3}R^{d3}, NR^{c3}C(═NR^{e3})NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3}, wherein said C₁₋₆ alkyl, C₂₋₆ alkenyl, C₂₋₆ alkynyl, C₁₋₆ haloalkyl, and C₃₋₄ cycloalkyl of R³ are each optionally substituted with 1, 2, or 3 substituents independently selected from halo, C₁₋₄ alkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, C(═NR^{e3})R^{b3}, C(═NR^{e3})NR^{c3}R^{d3}, NR^{c3}C(═NR^{e3})NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3};

each R^{a}, R^{b}, R^{c}, R^{d}, R^{a1}, R^{b1}, R^{c1}, R^{d1}, R^{a2}, R^{b2}, R^{c2}, and R^{d2} is independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl, wherein said C₁₋₆ alkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{a}, R^{b}, R^{c}, R^{d}, R^{a1}, R^{b1}, R^{c1}, R^{d1}, R^{a2}, R^{b2}, R^{c2}, and R^{d2} is optionally substituted with 1, 2, 3, 4, or 5 substituents independently selected from halo, C₁₋₄ alkyl, C₁₋₄haloalkyl, C₁₋₆ haloalkyl, C₂₋₆ alkenyl, C₂₋₆ alkynyl, CN, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)NR^{c3}R^{d3}, NR^{c3}C(O)OR^{a3}, C(═NR^{e3})NR^{c3}R^{d3}, NR^{c3}C(═NR^{e3})NR^{c3}R^{d3}, S(O)R^{b3}, S(O)NR^{c3}R^{d3}, S(O)₂R^{b3}, NR^{c3}S(O)₂R^{b3}, NR^{c3}S(O)₂NR^{c3}R^{d3}, and S(O)₂NR^{c3}R^{d3};

each R^{a3}, R^{b3}, R^{c3}, and R^{d3} is independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-6 membered heteroaryl, and 4-7 membered heterocycloalkyl, wherein said C₁₋₆ alkyl, C₁₋₆ haloalkyl, C₂₋₆ alkenyl, C₂₋₆ alkynyl, C₆₋₁₀ aryl, C₃₋₇ cycloalkyl, 5-6 membered heteroaryl, and 4-7 membered heterocycloalkyl are each optionally substituted with 1, 2, or 3 substituents independently selected from OH, CN, amino, halo, C₁₋₆ alkyl, C₁₋₆ alkoxy, C₁₋₆ haloalkyl, and C₁₋₆ haloalkoxy;

each R^{e}, R^{e1}, R^{e2}, and R^{e3} is independently selected from H, C₁₋₄ alkyl, and CN;

n is 0, 1, 2, or 3; and

m is 0, 1 or 2.

In some embodiments, X² is CR². In some embodiments, X² is N.

In some embodiments, X³ is CR³. In some embodiments, X³ is N.

In some embodiments, X⁴ is CR⁴. In some embodiments, X⁴ is N. In some embodiments, X⁴ is CH or N. In some embodiments, X⁴ is CH.

In some embodiments, A is selected from Cy¹, Cy¹-C₁₋₄ alkyl-, halo, C₁₋₆ alkyl, C₂₋₆ alkenyl, C₂₋₆ alkynyl, C₁₋₆ haloalkyl, CN, NO₂, OR^{a}, SR^{a}, C(O)R^{b}, C(O)NR^{e}R^{d}, C(O)OR^{a}, OC(O)R^{b}, OC(O)NR^{c}R^{d}, C(═NR^{e})NR^{c}R^{d}, NR^{c}C(═NR^{e})NR^{c}R^{d}, NR^{c}R^{d}, NR^{c}C(O)R^{b}, NR^{c}C(O)OR^{a}, NR^{c}C(O)NR^{c}R^{d}, NR^{c}S(O)R^{b}, NR^{c}S(O)₂R^{b}, NR^{c}S(O)₂NR^{c}R^{d}, S(O)R^{b}, S(O)NR^{e}R^{d}, S(O)₂R^{b}, and S(O)₂NR^{e}R^{d}; wherein said C₁₋₆ alkyl, C₂₋₆ alkenyl, C₂₋₆ alkynyl, and C₁₋₆ haloalkyl of A are each optionally substituted with 1, 2, 3, 4, or 5 substituents independently selected from halo, C₁₋₆ alkyl, C₂₋₆ alkenyl, C₂₋₆ alkynyl, C₁₋₆ haloalkyl, C₃₋₇ cycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, C(═NR^{e})NR^{c}R^{d}, NR^{c}C(═NR^{e})NR^{c}R^{d}, NR^{c}R^{d}, NR^{c}C(O)R^{b}, NR^{c}C(O)OR^{a}, NR^{c}C(O)NR^{c}R^{d}, NR^{c}S(O)R^{b}, NR^{c}S(O)₂R^{b}, NR^{c}S(O)₂NR^{c}R^{d}, S(O)R^{b}, S(O)NR^{c}R^{d}, S(O)₂R^{b}, and S(O)₂NR^{c}R^{d}.

In some embodiments, A is selected from Cy¹, halo, C₁₋₆ alkyl, C₁₋₆ haloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)OR^{a}, NR^{c}C(O)NR^{c}R^{d}, NR^{c}S(O)₂R^{b}, NR^{c}S(O)₂NR^{c}R^{d}, S(O)₂R^{b}, and S(O)₂NR^{e}R^{d}; wherein said C₁₋₆ alkyl and C₁₋₆ haloalkyl of A are each optionally substituted with 1, 2, 3, 4, or 5 substituents independently selected from halo, C₁₋₆ alkyl, C₁₋₆ haloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)OR^{a}, NR^{c}C(O)NR^{c}R^{d}, NR^{c}S(O)₂R^{b}, NR^{c}S(O)₂NR^{c}R^{d}, S(O)₂R^{b}, and S(O)₂NR^{c}R^{d}.

In some embodiments, A is selected from Cy¹, Cy¹-C₁₋₄ alkyl-, Cy¹-C₂₋₄ alkenyl-, halo, C₁₋₆ alkyl, C₁₋₆ haloalkyl, C₂₋₆ alkenyl, and OR^{a}.

In some embodiments, A is selected from halo, C₁₋₆ alkyl, C₁₋₆ haloalkyl, C₂₋₆ alkenyl, and OR^{a}.

In some embodiments, A is selected from Cy¹, Cy¹-C₁₋₄ alkyl-, and Cy¹-C₂₋₄ alkenyl-.

In some embodiments, A is selected from Cy¹, halo, and C₁₋₆ alkyl.

In some embodiments, A is selected from halo and C₁₋₆ alkyl.

In some embodiments, A is selected from methyl and iodide.

In some embodiments, A is Cy¹.

In some embodiments, A is selected from cyclopropylmethyl, styryl, methyl, bromide, chloride, iodide, CF₃, prop-1-en-1-yl, and methoxy.

In some embodiments, A is selected from methyl, bromide, chloride, iodide, CF₃, prop-1-en-1-yl, and methoxy.

In some embodiments, A is selected from methyl, iodide, isoxazol-4-yl, oxazol-5-yl, 1-(difluoromethyl)-1H-pyrazol-4-yl, and furan-3-yl.

In some embodiments, Cy¹ is 5-14 membered heteroaryl optionally substituted by 1, 2, 3, 4, or 5 substituents independently selected from halo, C₁₋₆ alkyl, C₂₋₆ alkenyl, C₂₋₆ alkynyl, C₁₋₆ haloalkyl, C₆₋₁₀ aryl, C₃₋₁₀ cycloalkyl, 5-14 membered heteroaryl, 4-14 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)OR^{a}, NR^{c}C(O)NR^{c}R^{d}, C(═NR^{e})R^{b}, C(═NR^{e})NR^{c}R^{d}, NR^{c}C(═NR^{e})NR^{c}R^{d}, NR^{c}S(O)R^{b}, NR^{c}S(O)₂R^{b}, NR^{c}S(O)₂NR^{c}R^{d}, S(O)R^{b}, S(O)NR^{c}R^{d}, S(O)₂R^{b}, and S(O)₂NR^{c}R^{d}, wherein said substituents C₁₋₆ alkyl, C₂₋₆ alkenyl, C₂₋₆ alkynyl, C₁₋₆ haloalkyl, C₆₋₁₀ aryl, C₃₋₁₀ cycloalkyl, 5-14 membered heteroaryl, 4-14 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl are each optionally substituted by 1, 2, or 3 further substituents independently selected from halo, C₁₋₆ alkyl, C₂₋₆ alkenyl, C₂₋₆ alkynyl, C₁₋₆ haloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)OR^{a}, NR^{c}C(O)NR^{c}R^{d}, C(═NR^{e})R^{b}, C(═NR^{e})NR^{c}R^{d}, NR^{c}C(═NR^{e})NR^{c}R^{d}, NR^{c}S(O)R^{b}, NR^{c}S(O)₂R^{b}, NR^{c}S(O)₂NR^{c}R^{d}, S(O)R^{b}, S(O)NR^{c}R^{d}, S(O)₂R^{b}, and S(O)₂NR^{c}R^{d}.

In some embodiments, Cy¹ is 5-10 membered heteroaryl optionally substituted by 1, 2, 3, 4, or 5 substituents independently selected from halo, C₁₋₆ alkyl, C₂₋₆ alkenyl, C₂₋₆ alkynyl, C₁₋₆ haloalkyl, C₆₋₁₀ aryl, C₃₋₁₀ cycloalkyl, 5-14 membered heteroaryl, 4-14 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)OR^{a}, NR^{c}C(O)NR^{c}R^{d}, C(═NR^{e})R^{b}, C(═NR^{e})NR^{c}R^{d}, NR^{c}C(═NR^{e})NR^{c}R^{d}, NR^{c}S(O)R^{b}, NR^{c}S(O)₂R^{b}, NR^{c}S(O)₂NR^{c}R^{d}, S(O)R^{b}, S(O)NR^{c}R^{d}, S(O)₂R^{b}, and S(O)₂NR^{c}R^{d}, wherein said substituents C₁₋₆ alkyl, C₂₋₆ alkenyl, C₂₋₆ alkynyl, C₁₋₆ haloalkyl, C₆₋₁₀ aryl, C₃₋₁₀ cycloalkyl, 5-14 membered heteroaryl, 4-14 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl are each optionally substituted by 1, 2, or 3 further substituents independently selected from halo, C₁₋₆ alkyl, C₂₋₆ alkenyl, C₂₋₆ alkynyl, C₁₋₆ haloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)OR^{a}, NR^{c}C(O)NR^{c}R^{d}, C(═NR^{e})R^{b}, C(═NR^{e})NR^{c}R^{d}, NR^{c}C(═NR^{e})NR^{c}R^{d}, NR^{c}S(O)R^{b}, NR^{c}S(O)₂R^{b}, NR^{c}S(O)₂NR^{c}R^{d}, S(O)R^{b}, S(O)NR^{c}R^{d}, S(O)₂R^{b}, and S(O)₂NR^{c}R^{d}.

In some embodiments, Cy¹ is 5-10 membered heteroaryl optionally substituted by 1, 2, 3, 4, or 5 substituents independently selected from halo, C₁₋₆ alkyl, C₁₋₆ haloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)OR^{a}, NR^{c}C(O)NR^{c}R^{d}, NR^{c}S(O)₂R^{b}, NR^{c}S(O)₂NR^{c}R^{d}, S(O)₂R^{b}, and S(O)₂NR^{c}R^{d}.

In some embodiments, Cy¹ is C₃₋₁₀ cycloalkyl, C₆₋₁₀ aryl, or 5-10 membered heteroaryl, each optionally substituted by 1, 2, 3, 4, or 5 substituents independently selected from 4-14 membered heterocycloalkyl, halo, C₁₋₆ alkyl, C₁₋₆ haloalkyl, CN, NO₂, OR^{a}, SR^{a}, C(O)R^{b}, C(O)NR^{c}R^{d}, C(O)OR^{a}, C(O)N(R^{c})OR^{a}, OC(O)R^{b}, OC(O)NR^{c}R^{d}, NR^{c}R^{d}, NR^{c}C(O)R^{b}, NR^{c}C(O)OR^{a}, NR^{c}C(O)NR^{c}R^{d}, NR^{c}S(O)₂R^{b}, NR^{c}S(O)₂NR^{c}R^{d}, S(O)₂R^{b}, and S(O)₂NR^{c}R^{d}.

In some embodiments, Cy¹ is C₃₋₁₀ cycloalkyl, C₆₋₁₀ aryl, or 5-10 membered heteroaryl, each optionally substituted by 1 or 2 substituents independently selected from 4-14 membered heterocycloalkyl, halo, C₁₋₆ alkyl, C₁₋₆ haloalkyl, CN, NO₂, OR^{a}, SR^{a}, C(O)R^{b}, C(O)NR^{c}R^{d}, C(O)OR^{a}, C(O)N(R^{c})OR^{a}, OC(O)R^{b}, OC(O)NR^{c}R^{d}, NR^{c}R^{d}, NR^{c}C(O)R^{b}, NR^{c}C(O)OR^{a}, NR^{c}C(O)NR^{c}R^{d}, NR^{c}S(O)₂R^{b}, NR^{c}S(O)₂NR^{c}R^{d}, S(O)₂R^{b}, and S(O)₂NR^{c}R^{d}.

In some embodiments, Cy¹ is 5-10 membered heteroaryl optionally substituted by 1 or 2 substituents independently selected from halo, C₁₋₆ alkyl, C₁₋₆ haloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)OR^{a}, NR^{c}C(O)NR^{c}R^{d}, NR^{c}S(O)₂R^{b}, NR^{c}S(O)₂NR^{c}R^{d}, S(O)₂R^{b}, and S(O)₂NR^{c}R^{d}.

In some embodiments, Cy¹ is C₃₋₆ cycloalkyl, phenyl, or 5-6 membered heteroaryl, each optionally substituted by 1 or 2 substituents independently selected from 4-14 membered heterocycloalkyl, halo, C₁₋₆ alkyl, C₁₋₆ haloalkyl, CN, NO₂, OR^{a}, SR^{a}, C(O)R^{b}, C(O)NR^{c}R^{d}, C(O)OR^{a}, C(O)N(R^{c})OR^{a}, OC(O)R^{b}, OC(O)NR^{c}R^{d}, NR^{c}R^{d}, NR^{c}C(O)R^{b}, NR^{c}C(O)OR^{a}, NR^{c}C(O)NR^{c}R^{d}, NR^{c}S(O)₂R^{b}, NR^{c}S(O)₂NR^{c}R^{d}, S(O)₂R^{b}, and S(O)₂NR^{c}R^{d}.

In some embodiments, Cy¹ is phenyl or 5-6 membered heteroaryl, each optionally substituted by 1 or 2 substituents independently selected from 4-14 membered heterocycloalkyl, halo, C₁₋₆ alkyl, C₁₋₆ haloalkyl, CN, NO₂, OR^{a}, SR^{a}, C(O)R^{b}, C(O)NR^{e}R^{d}, C(O)OR^{a}, C(O)N(R^{c})OR^{a}, OC(O)R^{b}, OC(O)NR^{c}R^{d}, NR^{c}R^{d}, NR^{c}C(O)R^{b}, NR^{c}C(O)OR^{a}, NR^{c}C(O)NR^{c}R^{d}, NR^{c}S(O)₂R^{b}, NR^{c}S(O)₂NR^{c}R^{d}, S(O)₂R^{b}, and S(O)₂NR^{c}R^{d}.

In some embodiments, Cy¹ is phenyl optionally substituted by 1 or 2 substituents independently selected from 4-14 membered heterocycloalkyl, halo, C₁₋₆ alkyl, C₁₋₆ haloalkyl, CN, NO₂, OR^{a}, SR^{a}, C(O)R^{b}, C(O)NR^{c}R^{d}, C(O)OR^{a}, C(O)N(R^{c})OR^{a}, OC(O)R^{b}, OC(O)NR^{c}R^{d}, NR^{c}R^{d}, NR^{c}C(O)R^{b}, NR^{c}C(O)OR^{a}, NR^{c}C(O)NR^{c}R^{d}, NR^{c}S(O)₂R^{b}, NR^{c}S(O)₂NR^{c}R^{d}, S(O)₂R^{b}, and S(O)₂NR^{c}R^{d}.

In some embodiments, Cy¹ is 5-6 membered heteroaryl optionally substituted by 1 or 2 substituents independently selected from halo, C₁₋₆ alkyl, C₁₋₆ haloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)OR^{a}, NR^{c}C(O)NR^{c}R^{d}, NR^{c}S(O)₂R^{b}, NR^{c}S(O)₂NR^{c}R^{d}, S(O)₂R^{b}, and S(O)₂NR^{c}R^{d}.

In some embodiments, Cy¹ is 5-membered heteroaryl optionally substituted by 1 or 2 substituents independently selected from halo, C₁₋₆ alkyl, C₁₋₆ haloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)OR^{a}, NR^{c}C(O)NR^{c}R^{d}, NR^{c}S(O)₂R^{b}, NR^{c}S(O)₂NR^{c}R^{d}, S(O)₂R^{b}, and S(O)₂NR^{c}R^{d}.

In some embodiments, Cy¹ is 6-membered heteroaryl optionally substituted by 1 or 2 substituents independently selected from halo, C₁₋₆ alkyl, C₁₋₆ haloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)OR^{a}, NR^{c}C(O)NR^{c}R^{d}, NR^{c}S(O)₂R^{b}, NR^{c}S(O)₂NR^{c}R^{d}, S(O)₂R^{b}, and S(O)₂NR^{c}R^{d}.

In some embodiments, Cy¹ is 5-6 membered heteroaryl optionally substituted with C₁₋₆ haloalkyl.

In some embodiments, Cy¹ is 5-membered heteroaryl optionally substituted with C₁₋₆ haloalkyl.

In some embodiments, Cy¹ is selected from isoxazolyl, oxazolyl, pyrazolyl, and furanyl, each of which is optionally substituted by 1 or 2 substituents independently selected from halo, C₁₋₆ alkyl, C₁₋₆ haloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)OR^{a}, NR^{c}C(O)NR^{c}R^{d}, NR^{c}S(O)₂R^{b}, NR^{c}S(O)₂NR^{c}R^{d}, S(O)₂R^{b}, and S(O)₂NR^{c}R^{d}.

In some embodiments, Cy¹ is selected from phenyl, pyridinyl, isoxazolyl, oxazolyl, pyrazolyl, furanyl, thiazolyl, cyclohexyl, oxo-1,2-dihydropyridinyl, cyclohex-1-en-1-yl, 1H,2′H-[3,6′-biindazol]-yl, benzo[d]thiazolyl, 1H-indolyl, 6-oxo-1,6-dihydropyridin-3-yl, cyclopent-1-en-1-yl, benzo[d]thiazolyl, benzo[d][1,3]dioxolyl, 2-oxoindolinyl, 1H,2′H-[3,5′-biindazol]-5-yl, 2,3-dihydrobenzo[b][1,4]dioxinyl, 1,4-dioxaspiro[4.5]dec-7-enyl, 3,6-dihydro-2H-pyran-4-yl, 1,2,3,6-tetrahydropyridin-4-yl, 5,6-dihydro-4H-pyrrolo[1,2-b]pyrazolyl, 2-oxo-1,2-dihydropyridin-4-yl, and 1,2-oxazolyl, each of which is optionally substituted by 1 or 2 substituents independently selected from 5-6 membered heterocycloalkyl, C₃₋₆ cycloalkyl, halo, C₁₋₆ alkyl, C₁₋₆ haloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)OR^{a}, NR^{c}C(O)NR^{c}R^{d}, NR^{c}S(O)₂R^{b}, NR^{c}S(O)₂NR^{c}R^{d}, S(O)₂R^{b}, and S(O)₂NR^{c}R^{d}.

In some embodiments, Cy¹ is selected from isoxazolyl, oxazolyl, pyrazolyl, and furanyl, each of which is optionally substituted by C₁₋₆ haloalkyl.

In some embodiments, Cy¹ is selected from isoxazol-4-yl, oxazol-5-yl, 1-(difluoromethyl)-1H-pyrazol-4-yl, and furan-3-yl.

In some embodiments, Cy¹ is selected from isoxazol-4-yl, oxazol-5-yl, 1-(difluoromethyl)-1H-pyrazol-4-yl, furan-3-yl, 4-carboxyphenyl, thiazol-5-yl, 1H-2-yl, 1-methyl-1H-2-yl, 2-methyloxazol-5-yl, 1H-pyrazol-5-yl, 3-methylisothiazol-5-yl, pyrazin-2-yl, 2-morpholinopyridin-4-yl, 2-methoxypyridin-4-yl, cyclopropyl, cyclohexyl, 1-methyl-2-oxo-1,2-dihydropyridin-3-yl, 2′-methyl-1H,2′H-[3,6′-biindazol]-5-yl, 3-(methylsulfonyl)phenyl, 3,5-dimethoxyphenyl, benzo[d]thiazol-6-yl, 1H-indol-6-yl, 1-methyl-6-oxo-1,6-dihydropyridin-3-yl, 4-cyanophenyl, pyridin-4-yl, cyclopent-1-en-1-yl, 3-carboxy-4-fluorophenyl, benzo[d]thiazol-5-yl, 3-(difluoromethyl)phenyl, 3-(methoxycarbamoyl)phenyl, 4-nitrophenyl, 3,4-dimethoxyphenyl, 4-morpholinophenyl, 4-methoxy-3-methylphenyl, 4-(methylsulfonyl)phenyl, 5-cyclopropylpyridin-3-yl, benzo[d][1,3]dioxol-5-yl, 1H-indol-6-yl, 1-(tert-butoxycarbonyl)-1H-pyrrol-2-yl, 4-(morpholine-4-carbonyl)phenyl, 2-oxoindolin-6-yl, 2′-methyl-1H,2′H-[3,5′-biindazol]-5-yl, 2,3-dihydrobenzo[b][1,4]dioxin-6-yl, 3-acetamidophenyl, 3-(dimethylcarbamoyl)phenyl, 1,4-dioxaspiro[4.5]dec-7-en-8-yl, 3,6-dihydro-2H-pyran-4-yl, 3-cyanophenyl, 2-methylpyridin-4-yl, 6-cyanopyridin-3-yl, 4-methoxyphenyl, 1-methyl-1,2,3,6-tetrahydropyridin-4-yl, 4-bromophenyl, 5,6-dihydro-4H-pyrrolo[1,2-b]pyrazol-3-yl, 1-methyl-2-oxo-1,2-dihydropyridin-4-yl, pyridin-3-yl, 5-methylpyridin-3-yl, 2-ethylpyridin-4-yl, 2-methoxypyridin-4-yl, and 1,2-oxazol-4-yl.

In some embodiments, L is 0 or NR^{N}. In some embodiments, L is O or NH. In some embodiments, L is 0. In some embodiments, L is NR^{N}. In some embodiments, L is NH. In some embodiments, R^{N} is H. In some embodiments, L is NCH₃. In some embodiments, R^{N} is CH₃. In some embodiments, R^{N} is H or CH₃.

In some embodiments, Ring D is C₃₋₇ cycloalkyl fused with Ring E. In some embodiments, Ring D is C₅₋₇ cycloalkyl fused with Ring E.

In some embodiments, Ring D is cyclopentyl or cyclohexyl fused with Ring E. In some embodiments, Ring D is cyclopentyl fused with Ring E. In some embodiments, Ring D is cyclohexyl fused with Ring E.

In some embodiments, Ring D is tetrahydropyranyl, cyclopentyl, cyclohexyl, or cycloheptyl, each of which is fused with Ring E.

In some embodiments, Ring D is cyclopentyl, cyclohexyl, or cycloheptyl, each of which is fused with Ring E.

In some embodiments, Ring D is a tetrahydropyranyl group fused with Ring E.

In some embodiments, Ring E is phenyl or a 6-membered heteroaryl group, fused with Ring D.

In some embodiments, Ring E is phenyl or a 5-6 membered heteroaryl group, each of which is fused with Ring D.

In some embodiments, Ring E is phenyl fused with Ring D.

In some embodiments, Ring E is a 5-6 membered heteroaryl group fused with Ring D.

In some embodiments, Ring E is pyridinyl fused with Ring D.

In some embodiments, Ring E is phenyl or pyridinyl fused with Ring D.

In some embodiments, Ring E is phenyl, pyridinyl, pyridazinyl, oxazolyl, thiazolyl, or pyrazinyl, each of which is fused with Ring D.

In some embodiments, Ring E is pyridinyl, pyridazinyl, oxazolyl, thiazolyl, or pyrazinyl, each of which is fused with Ring D.

In some embodiments, m is 0. In some embodiments, m is 2. In some embodiments, m is 0, 1, or 2.

In some embodiments, n is 0 or 1. In some embodiments, n is 0. In some embodiments, n is 1. In some embodiments, n is 2. In some embodiments, n is 0, 1, or 2.

In some embodiments, each R¹ and R^{1a} is independently selected from H, halo, C₁₋₆ alkyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, NR^{c1}S(O)₂NR^{c1}R^{d1}, S(O)₂R^{b1}, and S(O)₂NR^{c1}R^{d1}.

In some embodiments, each R¹ is independently selected from H, halo, C₁₋₆ alkyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, NR^{c1}S(O)₂NR^{c1}R^{d1}, S(O)₂R^{b1}, and S(O)₂NR^{c1}R^{d1}.

In some embodiments, each R^{1a} is independently selected from H, halo, C₁₋₆ alkyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, NR^{c1}S(O)₂NR^{c1}R^{d1}, S(O)₂R^{b1}, and S(O)₂NR^{c1}R^{d1}.

In some embodiments, each R¹ is independently selected from halo, C₁₋₆ alkyl, C₁₋₆ haloalkyl, and OR^{a1}.

In some embodiments, each R¹ is independently selected from halo and C₁₋₆ alkyl.

In some embodiments, each R¹ is independently selected from F and methyl.

In some embodiments, each R¹ is independently selected from F, Cl, Br, methyl, CF₃, OCH₃, and CHF₂

In some embodiments, each R^{1a} is independently selected from H, halo, and C₁₋₆ alkyl.

In some embodiments, each R^{1a} is independently selected from H, D, halo, and C₁₋₆ alkyl.

In some embodiments, R^{1a} is H, D, F, or methyl.

In some embodiments, each R^{1a} is H.

In some embodiments, two R^{1a} groups together with the atoms to which they are attached form a C₃₋₇ cycloalkyl group.

In some embodiments, two R^{1a} groups together with the atoms to which they are attached form a cyclopropyl group.

In some embodiments, R² and R⁴ are each independently selected from H, halo, C₁₋₆ alkyl, CN, NO₂, OR², SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a}2, NR^{c2}C(O)NR^{c2}R^{d2}, NR^{c2}S(O)₂R^{b2}, NR^{c2}S(O)₂NR^{c2}R^{d2}, S(O)₂R^{b2}, and S(O)₂NR^{c2}R^{d2}, wherein said C₁₋₆ alkyl is optionally substituted with 1, 2, 3, 4, or 5 substituents independently selected from C₁₋₆ alkyl, halo, CN, NO₂, OR^{a2}, SR^{a}2, C(O)R^{b2}, C(O)NR^{c2}R^{d2}, C(O)OR^{a}2, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)₂R^{b2}, NR^{c2}S(O)₂NR^{c2}R^{d2}, S(O)₂R^{b2}, and S(O)₂NR^{c2}R^{d2}.

In some embodiments, R² and R⁴ are each independently selected from H, halo, C₁₋₆ alkyl, CN, and OR^{a}2.

In some embodiments, R² is H.

In some embodiments, R⁴ is H.

In some embodiments, R³ is selected from H, halo, C₁₋₆ alkyl, C₁₋₆ haloalkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)₂R^{b3}, NR^{c3}S(O)₂NR^{c3}R^{d3}, S(O)₂R^{b3}, and S(O)₂NR^{c3}R^{d3}, wherein said C₁₋₆ alkyl and C₁₋₆ haloalkyl of R³ are each optionally substituted with 1, 2, or 3 substituents independently selected from halo, C₁₋₄ alkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)₂R^{b3}, NR^{c3}S(O)₂NR^{c3}R^{d3}, S(O)₂R^{b3}, and S(O)₂NR^{c3}R^{d3}.

In some embodiments, R³ is selected from H, halo, C₁₋₆ alkyl, and C₁₋₆ haloalkyl.

In some embodiments, R³ is H.

In some embodiments, each R^{a}, R^{b}, R^{c}, R^{d}, R^{a1}, R^{b1}, R^{c1}, R^{d1}, R^{a2}, R^{b2}, R^{c2}, and R^{d2} is independently selected from H, C₁₋₆ alkyl, and C₁₋₆ haloalkyl, wherein said C₁₋₆ alkyl of R^{a}, R^{b}, R^{c}, R^{d}, R^{a1}, R^{b1}, R^{c1}, R^{d1}, R^{a2}, R^{b2}, R^{c2}, and R^{d2} is optionally substituted with 1, 2, 3, 4, or 5 substituents independently selected from halo, C₁₋₄ alkyl, C₁₋₄haloalkyl, C₁₋₆ haloalkyl, CN, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)NR^{c3}R^{d3}, NR^{c3}C(O)OR^{a3}, S(O)₂R^{b3}, NR^{c3}S(O)₂R^{b3}, NR^{c3}S(O)₂NR^{c3}R^{d3}, and S(O)₂NR^{c3}R^{d3}.

In some embodiments, each R^{a}, R^{b}, R^{c}, R^{d}, R^{a1}, R^{b1}, R^{c1}, and R^{d1} is independently selected from H, C₁₋₆ alkyl, and C₁₋₆ haloalkyl, wherein said C₁₋₆ alkyl of R^{a}, R^{b}, R^{c}, R^{d}, R^{a1}, R^{b1}, R^{c1}, and R^{d1} is optionally substituted with 1, 2, 3, 4, or 5 substituents independently selected from halo, C₁₋₄ alkyl, C₁₋₄haloalkyl, C₁₋₆ haloalkyl, CN, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)NR^{c3}R^{d3}, NR^{c3}C(O)OR^{a3}, S(O)₂R^{b3}, NR^{c3}S(O)₂R^{b3}, NR^{c3}S(O)₂NR^{c3}R^{d3}, and S(O)₂NR^{c3}R^{d3}.

In some embodiments, each R^{a}, R^{b}, R^{c}, R^{d}, R^{a1}, R^{b1}, R^{c1}, R^{d1}, R^{a2}, R^{b2}, R^{c2}, and R^{d2} is independently selected from H, C₁₋₆ alkyl, and C₁₋₆ haloalkyl.

In some embodiments, each R^{a}, R^{b}, R^{c}, R^{d}, R^{a1}, R^{b1}, R^{c1}, and R^{d1} is independently selected from H, C₁₋₆ alkyl, and C₁₋₆ haloalkyl.

In some embodiments, each R^{a3}, R^{b3}, R^{c3}, and R^{d3} is independently selected from H, C₁₋₆ alkyl, and C₁₋₆ haloalkyl, wherein said C₁₋₆ alkyl, and C₁₋₆ haloalkyl are each optionally substituted with 1, 2, or 3 substituents independently selected from OH, CN, amino, halo, C₁₋₆ alkyl, C₁₋₆ alkoxy, C₁₋₆ haloalkyl, and C₁₋₆ haloalkoxy.

In some embodiments, each R^{a3}, R^{b3}, R^{c3}, and R^{d3} is independently selected from H, C₁₋₆ alkyl, and C₁₋₆ haloalkyl.

In some embodiments, the compound is of Formula II:

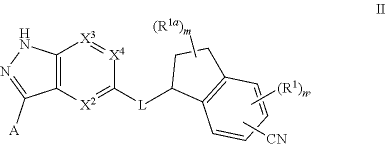

or a pharmaceutically acceptable salt thereof.

In some embodiments, the compound is of Formula III:

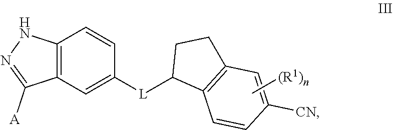

or a pharmaceutically acceptable salt thereof.

In some embodiments, the compound is of Formula IVa:

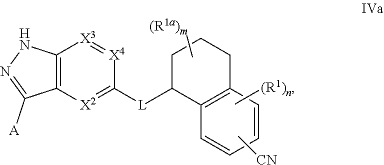

or a pharmaceutically acceptable salt thereof.

In some embodiments, the compound is of Formula IVb:

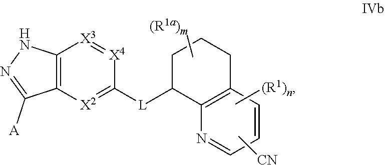

or a pharmaceutically acceptable salt thereof.

In some embodiments, the compound is of Formula Va:

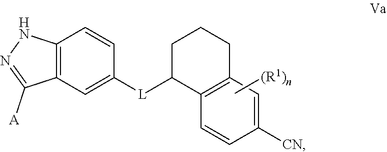

or a pharmaceutically acceptable salt thereof.

In some embodiments, the compound is of Formula Vb:

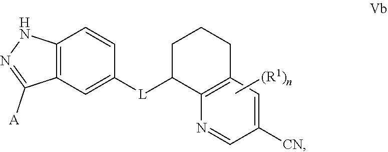

or a pharmaceutically acceptable salt thereof.

In some embodiments, the compound is of Formula VIa:

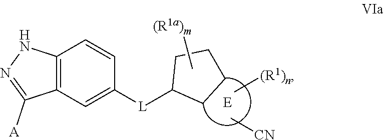

or a pharmaceutically acceptable salt thereof. In some embodiments, the compound is Formula VIb:

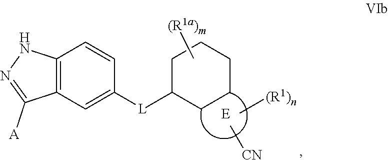

or a pharmaceutically acceptable salt thereof.

In some embodiments, provided herein is a compound of Formula I, or a pharmaceutically acceptable salt thereof, wherein:

A is selected from Cy¹, halo, and C₁₋₆ alkyl;

L is O or NH;

X² is CR²;

X³ is CR³;

X⁴ is N or CR⁴;

Ring D is cyclopentyl or cyclohexyl group fused with Ring E;

Ring E is phenyl or a 6-membered heteroaryl group, fused with Ring D;

Cy¹ is 5-6 membered heteroaryl optionally substituted by 1 or 2 substituents independently selected from halo, C₁₋₆ alkyl, C₁₋₆ haloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)OR^{a}, NR^{c}C(O)NR^{c}R^{d}, NR^{c}S(O)₂R^{b}, NR^{c}S(O)₂NR^{c}R^{d}, S(O)₂R^{b}, and S(O)₂NR^{c}R^{d};

each R¹ and R^{1a} is independently selected from H, halo, C₁₋₆ alkyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, NR^{c1}S(O)₂NR^{c1}R^{d1}, S(O)₂R^{b1}, and S(O)₂NR^{c1}R^{d1};

R², R³, and R⁴ are each H;

each R^{a}, R^{b}, R^{c}, R^{d}, R^{a1}, R^{b1}, R^{c1}, and R^{d1} is independently selected from H, C₁₋₆ alkyl, and C₁₋₆ haloalkyl, wherein said C₁₋₆ alkyl of R^{a}, R^{b}, R^{c}, R^{d}, R^{a1}, R^{b1}, R^{c1}, and R^{d1} is optionally substituted with 1, 2, 3, 4, or 5 substituents independently selected from halo, C₁₋₄ alkyl, C₁₋₄ haloalkyl, C₁₋₆ haloalkyl, CN, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)NR^{c3}R^{d3}, NR^{c3}C(O)OR^{a3}, S(O)₂R^{b3}, NR^{c3}S(O)₂R^{b3}, NR^{c3}S(O)₂NR^{c3}R^{d3}, and S(O)₂NR^{c1}R^{d1};

each R^{a3}, R^{b3}, R^{c3}, and R^{d3} is independently selected from H, C₁₋₆ alkyl, and C₁₋₆ haloalkyl, wherein said C₁₋₆ alkyl, and C₁₋₆ haloalkyl are each optionally substituted with 1, 2, or 3 substituents independently selected from OH, CN, amino, halo, C₁₋₆ alkyl, C₁₋₆ alkoxy, C₁₋₆ haloalkyl, and C₁₋₆ haloalkoxy;

n is 0, or 1; and

m is 0.

In some embodiments, provided herein is a compound of Formula I, or a pharmaceutically acceptable salt thereof, wherein:

A is selected from Cy¹, Cy¹-C₁₋₄ alkyl-, Cy¹-C₂₋₄ alkenyl-, halo, C₁₋₆ alkyl, C₁₋₆ haloalkyl, C₂₋₆ alkenyl, and OR^{a};

L is O, NH, or NCH₃;

X² is N or CR²;

X³ is CR³;

X⁴ is N or CR⁴;

Ring D is cyclopentyl, cyclohexyl, cycloheptyl, or tetrahydropyranyl, each of which is fused with Ring E;

Ring E is phenyl or a 5-6 membered heteroaryl group, each of which is fused with Ring D;

Cy¹ is C₃₋₆ cycloalkyl, phenyl, or 5-6 membered heteroaryl, each optionally substituted by 1 or 2 substituents independently selected from 4-14 membered heterocycloalkyl, halo, C₁₋₆ alkyl, C₁₋₆ haloalkyl, CN, NO₂, OR^{a}, SR^{a}, C(O)R^{b}, C(O)NR^{c}R^{d}, C(O)OR^{a}, C(O)N(R^{c})OR^{a}, OC(O)R^{b}, OC(O)NR^{c}R^{d}, NR^{c}R^{d}, NR^{c}C(O)R^{b}, NR^{c}C(O)OR^{a}, NR^{c}C(O)NR^{c}R^{d}, NR^{c}S(O)₂R^{b}, NR^{c}S(O)₂NR^{c}R^{d}, S(O)₂R^{b}, and S(O)₂NR^{c}R^{d};

each R¹ and R^{1a} is independently selected from H, D, halo, C₁₋₆ alkyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, NR^{c1}S(O)₂NR^{c1}R^{d1}, S(O)₂R^{b1}, and S(O)₂NR^{c1}R^{d1};

or two R^{1a} groups together with the atoms to which they are attached form a C₃₋₆ cycloalkyl group;

R², R³, and R⁴ are each H;

each R^{a}, R^{b}, R^{c}, R^{d}, R^{a1}, R^{b1}, R^{c1}, and R^{d1} is independently selected from H, C₁₋₆ alkyl, and C₁₋₆ haloalkyl, wherein said C₁₋₆ alkyl of R^{a}, R^{b}, R^{c}, R^{d}, R^{a1}, R^{b1}, R^{c1}, and R^{d1} is optionally substituted with 1, 2, 3, 4, or 5 substituents independently selected from halo, C₁₋₄ alkyl, C₁₋₄ haloalkyl, C₁₋₆ haloalkyl, CN, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)NR^{c3}R^{d3}, NR^{c3}C(O)OR^{a3}, S(O)₂R^{b3}, NR^{c3}S(O)₂R^{b3}, NR^{c3}S(O)₂NR^{c3}R^{d3}, and S(O)₂NR^{c3}R^{d3};

each R^{a3}, R^{b3}, R^{c3}, and R^{d3} is independently selected from H, C₁₋₆ alkyl, and C₁₋₆ haloalkyl, wherein said C₁₋₆ alkyl, and C₁₋₆ haloalkyl are each optionally substituted with 1, 2, or 3 substituents independently selected from OH, CN, amino, halo, C₁₋₆ alkyl, C₁₋₆ alkoxy, C₁₋₆ haloalkyl, and C₁₋₆ haloalkoxy;

n is 0, 1, or 2; and

m is 0, 1, or 2.

Provided herein is a compound selected from the following:
(S)-1-((3-Methyl-1H-indazol-5-yl)oxy)-2,3-dihydro-1H-indene-5-carbonitrile;
(R)-1-((3-Methyl-1H-indazol-5-yl)oxy)-2,3-dihydro-1H-indene-5-carbonitrile;
(S)-8-((3-(Isoxazol-4-yl)-1H-indazol-5-yl)oxy)-5,6,7,8-tetrahydroquinoline-3-carbonitrile;
(R)-8-((3-(Isoxazol-4-yl)-1H-indazol-5-yl)oxy)-5,6,7,8-tetrahydroquinoline-3-carbonitrile;
(S)-1-((3-(Oxazol-5-yl)-1H-indazol-5-yl)oxy)-2,3-dihydro-1H-indene-5-carbonitrile;
(R)-1-((3-(Oxazol-5-yl)-1H-indazol-5-yl)oxy)-2,3-dihydro-1H-indene-5-carbonitrile;
(S)-1-((3-(1-(Difluoromethyl)-1H-pyrazol-4-yl)-1H-indazol-5-yl)oxy)-2,3-dihydro-1H-indene-5-carbonitrile;
(R)-1-((3-(1-(Difluoromethyl)-1H-pyrazol-4-yl)-1H-indazol-5-yl)oxy)-2,3-dihydro-1H-indene-5-carbonitrile;
(S)-1-((3-(Isoxazol-4-yl)-1H-indazol-5-yl)oxy)-2,3-dihydro-1H-indene-5-carbonitrile;
(S)-1-((3-(Isoxazol-4-yl)-1H-indazol-5-yl)oxy)-2,3-dihydro-1H-indene-5-carbonitrile;
(S)-5-((3-(Isoxazol-4-yl)-1H-indazol-5-yl)amino)-5,6,7,8-tetrahydronaphthalene-2-carbonitrile;
(R)-5-((3-(Isoxazol-4-yl)-1H-indazol-5-yl)amino)-5,6,7,8-tetrahydronaphthalene-2-carbonitrile;
(S)-8-((3-(Isoxazol-4-yl)-1H-indazol-5-yl)amino)-5,6,7,8-tetrahydroquinoline-3-carbonitrile;
(R)-8-((3-(Isoxazol-4-yl)-1H-indazol-5-yl)amino)-5,6,7,8-tetrahydroquinoline-3-carbonitrile;
(S)-1-((3-(Isoxazol-4-yl)-1H-indazol-5-yl)amino)-2,3-dihydro-1H-indene-5-carbonitrile;
(R)-1-((3-(Isoxazol-4-yl)-1H-indazol-5-yl)amino)-2,3-dihydro-1H-indene-5-carbonitrile;
(S)-1-((3-(Oxazol-5-yl)-1H-indazol-5-yl)amino)-2,3-dihydro-1H-indene-5-carbonitrile;
(R)-1-((3-(Oxazol-5-yl)-1H-indazol-5-yl)amino)-2,3-dihydro-1H-indene-5-carbonitrile;
1-((3-(Furan-3-yl)-1H-indazol-5-yl)amino)-2,3-dihydro-1H-indene-5-carbonitrile;
(S)-1-((3-(Furan-3-yl)-1H-indazol-5-yl)amino)-2,3-dihydro-1H-indene-5-carbonitrile;
(R)-1-((3-(Furan-3-yl)-1H-indazol-5-yl)amino)-2,3-dihydro-1H-indene-5-carbonitrile
(S)-1-((3-(1-(Difluoromethyl)-1H-pyrazol-4-yl)-1H-indazol-5-yl)amino)-2,3-dihydro-1H-indene-5-carbonitrile;
(R)-1-((3-(1-(Difluoromethyl)-1H-pyrazol-4-yl)-1H-indazol-5-yl)amino)-2,3-dihydro-1H-indene-5-carbonitrile;
(S)-1-((3-Iodo-1H-indazol-5-yl)oxy)-2,3-dihydro-1H-indene-5-carbonitrile;
(S)-7-methyl-1-((3-(oxazol-5-yl)-1H-indazol-5-yl)oxy)-2,3-dihydro-1H-indene-5-carbonitrile;
(R)-7-methyl-1-((3-(oxazol-5-yl)-1H-indazol-5-yl)oxy)-2,3-dihydro-1H-indene-5-carbonitrile;
(S)-5-((3-iodo-1H-indazol-5-yl)amino)-5,6,7,8-tetrahydronaphthalene-2-carbonitrile;
(R)-5-((3-iodo-1H-indazol-5-yl)amino)-5,6,7,8-tetrahydronaphthalene-2-carbonitrile;
(S)-8-((3-(Oxazol-5-yl)-1H-indazol-5-yl)oxy)-5,6,7,8-tetrahydroquinoline-3-carbonitrile;
(R)-8-((3-(Oxazol-5-yl)-1H-indazol-5-yl)oxy)-5,6,7,8-tetrahydroquinoline-3-carbonitrile;
(S)-5-((3-(Isoxazol-4-yl)-1H-indazol-5-yl)oxy)-5,6,7,8-tetrahydronaphthalene-2-carbonitrile;
(R)-5-((3-(Isoxazol-4-yl)-1H-indazol-5-yl)oxy)-5,6,7,8-tetrahydronaphthalene-2-carbonitrile;
(S)-5-((3-(Oxazol-5-yl)-1H-indazol-5-yl)oxy)-5,6,7,8-tetrahydronaphthalene-2-carbonitrile;
(R)-5-((3-(Oxazol-5-yl)-1H-indazol-5-yl)oxy)-5,6,7,8-tetrahydronaphthalene-2-carbonitrile;
(S)-5-((3-Methyl-1H-indazol-5-yl)oxy)-5,6,7,8-tetrahydronaphthalene-2-carbonitrile;
(R)-5-((3-Methyl-1H-indazol-5-yl)oxy)-5,6,7,8-tetrahydronaphthalene-2-carbonitrile;
(S)-1-((3-(Oxazol-5-yl)-1H-pyrazolo[3,4-c]pyridin-5-yl)oxy)-2,3-dihydro-1H-indene-5-carbonitrile;
(R)-1-((3-(Oxazol-5-yl)-1H-pyrazolo[3,4-c]pyridin-5-yl)oxy)-2,3-dihydro-1H-indene-5-carbonitrile;
(S)-1-((3-(Oxazol-5-yl)-1H-pyrazolo[3,4-c]pyridin-5-yl)amino)-2,3-dihydro-1H-indene-5-carbonitrile
(R)-1-((3-(Oxazol-5-yl)-1H-pyrazolo[3,4-c]pyridin-5-yl)amino)-2,3-dihydro-1H-indene-5-carbonitrile
(S)-4-Methyl-1-((3-(oxazol-5-yl)-1H-indazol-5-yl)oxy)-2,3-dihydro-1H-indene-5-carbonitrile;
(R)-4-Methyl-1-((3-(oxazol-5-yl)-1H-indazol-5-yl)oxy)-2,3-dihydro-1H-indene-5-carbonitrile;
(S)-6-Fluoro-1-((3-(oxazol-5-yl)-1H-indazol-5-yl)oxy)-2,3-dihydro-1H-indene-5-carbonitrile;
(R)-6-Fluoro-1-((3-(oxazol-5-yl)-1H-indazol-5-yl)oxy)-2,3-dihydro-1H-indene-5-carbonitrile;
(S)-4-Fluoro-1-((3-(oxazol-5-yl)-1H-indazol-5-yl)oxy)-2,3-dihydro-1H-indene-5-carbonitrile;
(R)-4-Fluoro-1-((3-(oxazol-5-yl)-1H-indazol-5-yl)oxy)-2,3-dihydro-1H-indene-5-carbonitrile;
(S)-6-Fluoro-1-((3-(oxazol-5-yl)-1H-indazol-5-yl)amino)-2,3-dihydro-1H-indene-5-carbonitrile;
(R)-6-Fluoro-1-((3-(oxazol-5-yl)-1H-indazol-5-yl)amino)-2,3-dihydro-1H-indene-5-carbonitrile;
(S)-4-Fluoro-1-((3-(oxazol-5-yl)-1H-indazol-5-yl)amino)-2,3-dihydro-1H-indene-5-carbonitrile;
(R)-4-Fluoro-1-((3-(oxazol-5-yl)-1H-indazol-5-yl)amino)-2,3-dihydro-1H-indene-5-carbonitrile;
(S)-4-Methyl-1-((3-(oxazol-5-yl)-1H-indazol-5-yl)amino)-2,3-dihydro-1H-indene-5-carbonitrile;
(R)-4-Methyl-1-((3-(oxazol-5-yl)-1H-indazol-5-yl)amino)-2,3-dihydro-1H-indene-5-carbonitrile;
(S)-6-Methyl-1-((3-(oxazol-5-yl)-1H-indazol-5-yl)amino)-2,3-dihydro-1H-indene-5-carbonitrile; and
(R)-6-Methyl-1-((3-(oxazol-5-yl)-1H-indazol-5-yl)amino)-2,3-dihydro-1H-indene-5-carbonitrile;

or a pharmaceutically acceptable salt of any of the aforementioned.

Provided herein is a compound selected from:
6-Methyl-1-((3-(oxazol-5-yl)-1H-indazol-5-yl)oxy)-2,3-dihydro-1H-indene-5-carbonitrile;
1-((3-(Oxazol-5-yl)-1H-pyrazolo[4,3-b]pyridin-5-yl)amino)-2,3-dihydro-1H-indene-5-carbonitrile;
1-((3-(Oxazol-5-yl)-1H-pyrazolo[4,3-b]pyridin-5-yl)oxy)-2,3-dihydro-1H-indene-5-carbonitrile;
5-((3-(Oxazol-5-yl)-1H-pyrazolo[3,4-c]pyridin-5-yl)oxy)-5,6,7,8-tetrahydronaphthalene-2-carbonitrile;
5-((3-(Oxazol-5-yl)-1H-pyrazolo[3,4-c]pyridin-5-yl)amino)-5,6,7,8-tetrahydronaphthalene-2-carbonitrile;
6,6-Difluoro-7-((3-(oxazol-5-yl)-1H-indazol-5-yl)amino)-6,7-dihydro-5H-cyclopenta[b]pyridine-3-carbonitrile;
4-((3-(Oxazol-5-yl)-1H-indazol-5-yl)amino)-4,5,6,7-tetrahydrobenzo[d]oxazole-2-carbonitrile;
4-((3-(Oxazol-5-yl)-1H-indazol-5-yl)amino)-5,6-dihydro-4H-cyclopenta[d]oxazole-2-carbonitrile;
3-((3-(Oxazol-5-yl)-1H-indazol-5-yl)oxy)-2,3-dihydro-1H-indene-4-carbonitrile;
5-((3-(2-Morpholinopyridin-4-yl)-1H-indazol-5-yl)amino)-5,6,7,8-tetrahydronaphthalene-2-carbonitrile;
5-((3-(2-Morpholinopyridin-4-yl)-1H-indazol-5-yl)oxy)-5,6,7,8-tetrahydronaphthalene-2-carbonitrile;
1-((3-(2-Methoxypyridin-4-yl)-1H-indazol-5-yl)amino)-2,3-dihydro-1H-indene-5-carbonitrile;
7-((3-(Oxazol-5-yl)-1H-indazol-5-yl)oxy)-6,7-dihydro-5H-cyclopenta[c]pyridazine-3-carbonitrile;
2,2-Difluoro-1-((3-(oxazol-5-yl)-1H-indazol-5-yl)amino)-2,3-dihydro-1H-indene-5-carbonitrile;
5-((3-Methyl-1H-indazol-5-yl)amino)-5,6,7,8-tetrahydronaphthalene-2-carbonitrile;
5-((3-(Thiazol-5-yl)-1H-indazol-5-yl)amino)-5,6,7,8-tetrahydronaphthalene-2-carbonitrile;
5-((3-Cyclohexyl-1H-indazol-5-yl)amino)-5,6,7,8-tetrahydronaphthalene-2-carbonitrile;
5-((3-(1-Methyl-2-oxo-1,2-dihydropyridin-3-yl)-1H-indazol-5-yl)amino)-5,6,7,8-tetrahydronaphthalene-2-carbonitrile;
7-((3-(Oxazol-5-yl)-1H-indazol-5-yl)amino)-4,5,6,7-tetrahydrobenzo[d]thiazole-2-carbonitrile;
6-((3-(Oxazol-5-yl)-1H-indazol-5-yl)amino)-4b,5,5a,6-tetrahydrocyclopropa[3,4]cyclopenta[1,2-b]pyridine-3-carbonitrile;
8-((3-(1-(Difluoromethyl)-1H-pyrazol-4-yl)-1H-indazol-5-yl)amino)-5,6,7,8-tetrahydroquinoline-3-carbonitrile;
1-((3-(1-(Difluoromethyl)-1H-pyrazol-4-yl)-1H-indazol-5-yl)oxy)-4-methyl-2,3-dihydro-1H-indene-5-carbonitrile;
1-((3-(1-(Difluoromethyl)-1H-pyrazol-4-yl)-1H-indazol-5-yl)oxy)-4-fluoro-2,3-dihydro-1H-indene-5-carbonitrile;
1-((3-(1-(Difluoromethyl)-1H-pyrazol-4-yl)-1H-indazol-5-yl)amino)-4-methyl-2,3-dihydro-1H-indene-5-carbonitrile;
1-((3-(1-(Difluoromethyl)-1H-pyrazol-4-yl)-1H-indazol-5-yl)amino)-4-fluoro-2,3-dihydro-1H-indene-5-carbonitrile;
5-((3-(Cyclohex-1-en-1-yl)-1H-indazol-5-yl)amino)-5,6,7,8-tetrahydronaphthalene-2-carbonitrile;
4-(5-((6-Cyano-1,2,3,4-tetrahydronaphthalen-1-yl)amino)-1H-indazol-3-yl)benzoic acid;
5-((2′-Methyl-1H,2′H-[3,6′-biindazol]-5-yl)amino)-5,6,7,8-tetrahydronaphthalene-2-carbonitrile;
5-((3-(3-(Methylsulfonyl)phenyl)-1H-indazol-5-yl)amino)-5,6,7,8-tetrahydronaphthalene-2-carbonitrile;
5-((3-(3,5-Dimethoxyphenyl)-1H-indazol-5-yl)amino)-5,6,7,8-tetrahydronaphthalene-2-carbonitrile;
5-((3-(Benzo[d]thiazol-6-yl)-1H-indazol-5-yl)amino)-5,6,7,8-tetrahydronaphthalene-2-carbonitrile;
5-((3-(2-Methyloxazol-5-yl)-1H-indazol-5-yl)amino)-5,6,7,8-tetrahydronaphthalene-2-carbonitrile;
5-((3-(1H-Indol-6-yl)-1H-indazol-5-yl)amino)-5,6,7,8-tetrahydronaphthalene-2-carbonitrile;
5-((3-(1-Methyl-6-oxo-1,6-dihydropyridin-3-yl)-1H-indazol-5-yl)amino)-5,6,7,8-tetrahydronaphthalene-2-carbonitrile;
5-((3-(4-Cyanophenyl)-1H-indazol-5-yl)amino)-5,6,7,8-tetrahydronaphthalene-2-carbonitrile;
5-((3-(Pyridin-4-yl)-1H-indazol-5-yl)amino)-5,6,7,8-tetrahydronaphthalene-2-carbonitrile;
5-((3-(Cyclopent-1-en-1-yl)-1H-indazol-5-yl)amino)-5,6,7,8-tetrahydronaphthalene-2-carbonitrile;
5-(5-((6-Cyano-1,2,3,4-tetrahydronaphthalen-1-yl)amino)-1H-indazol-3-yl)-2-fluorobenzoic acid;
5-((3-(Benzo[d]thiazol-5-yl)-1H-indazol-5-yl)amino)-5,6,7,8-tetrahydronaphthalene-2-carbonitrile;
5-((3-(3-(Difluoromethyl)phenyl)-1H-indazol-5-yl)amino)-5,6,7,8-tetrahydronaphthalene-2-carbonitrile;
3-(5-((6-Cyano-1,2,3,4-tetrahydronaphthalen-1-yl)amino)-1H-indazol-3-yl)-N-methoxybenzamide;
5-((3-(4-Nitrophenyl)-1H-indazol-5-yl)amino)-5,6,7,8-tetrahydronaphthalene-2-carbonitrile;
5-((3-(3,4-Dimethoxyphenyl)-1H-indazol-5-yl)amino)-5,6,7,8-tetrahydronaphthalene-2-carbonitrile;
5-((3-(4-Morpholinophenyl)-1H-indazol-5-yl)amino)-5,6,7,8-tetrahydronaphthalene-2-carbonitrile;
5-((3-(4-Methoxy-3-methylphenyl)-1H-indazol-5-yl)amino)-5,6,7,8-tetrahydronaphthalene-2-carbonitrile;
5-((3-(4-(Methylsulfonyl)phenyl)-1H-indazol-5-yl)amino)-5,6,7,8-tetrahydronaphthalene-2-carbonitrile;
(E)-5-((3-(Prop-1-en-1-yl)-1H-indazol-5-yl)amino)-5,6,7,8-tetrahydronaphthalene-2-carbonitrile;
5-((3-(5-Cyclopropylpyridin-3-yl)-1H-indazol-5-yl)amino)-5,6,7,8-tetrahydronaphthalene-2-carbonitrile;
5-((3-(Benzo[d][1,3]dioxol-5-yl)-1H-indazol-5-yl)amino)-5,6,7,8-tetrahydronaphthalene-2-carbonitrile;
5-((3-(1H-Indol-5-yl)-1H-indazol-5-yl)amino)-5,6,7,8-tetrahydronaphthalene-2-carbonitrile;
5-((3-(1H-Pyrrol-2-yl)-1H-indazol-5-yl)amino)-5,6,7,8-tetrahydronaphthalene-2-carbonitrile;
tert-Butyl 2-(5-((6-cyano-1,2,3,4-tetrahydronaphthalen-1-yl)amino)-1H-indazol-3-yl)-1H-pyrrole-1-carboxylate;
5-((3-(4-(Morpholine-4-carbonyl)phenyl)-1H-indazol-5-yl)amino)-5,6,7,8-tetrahydronaphthalene-2-carbonitrile;
5-((3-(2-Oxoindolin-6-yl)-1H-indazol-5-yl)amino)-5,6,7,8-tetrahydronaphthalene-2-carbonitrile;
5-((2′-Methyl-1H,2′H-[3,5′-biindazol]-5-yl)amino)-5,6,7,8-tetrahydronaphthalene-2-carbonitrile;
5-((3-(2,3-Dihydrobenzo[b][1,4]dioxin-6-yl)-1H-indazol-5-yl)amino)-5,6,7,8-tetrahydronaphthalene-2-carbonitrile;
N-(3-(5-((6-Cyano-1,2,3,4-tetrahydronaphthalen-1-yl)amino)-1H-indazol-3-yl)phenyl)acetamide;
3-(5-((6-Cyano-1,2,3,4-tetrahydronaphthalen-1-yl)amino)-1H-indazol-3-yl)-N,N-dimethylbenzamide;
5-((3-(1,4-Dioxaspiro[4.5]dec-7-en-8-yl)-1H-indazol-5-yl)amino)-5,6,7,8-tetrahydronaphthalene-2-carbonitrile;
5-((3-(3,6-Dihydro-2H-pyran-4-yl)-1H-indazol-5-yl)amino)-5,6,7,8-tetrahydronaphthalene-2-carbonitrile;
5-((3-(3-Cyanophenyl)-1H-indazol-5-yl)amino)-5,6,7,8-tetrahydronaphthalene-2-carbonitrile;
5-((3-(2-Methylpyridin-4-yl)-1H-indazol-5-yl)amino)-5,6,7,8-tetrahydronaphthalene-2-carbonitrile;
5-(5-((6-Cyano-1,2,3,4-tetrahydronaphthalen-1-yl)amino)-1H-indazol-3-yl)picolinonitrile;
5-((3-(4-Methoxyphenyl)-1H-indazol-5-yl)amino)-5,6,7,8-tetrahydronaphthalene-2-carbonitrile;
(E)-5-((3-Styryl-1H-indazol-5-yl)amino)-5,6,7,8-tetrahydronaphthalene-2-carbonitrile;
5-((3-(1-Methyl-1,2,3,6-tetrahydropyridin-4-yl)-1H-indazol-5-yl)amino)-5,6,7,8-tetrahydronaphthalene-2-carbonitrile;
5-((3-(4-Bromophenyl)-1H-indazol-5-yl)amino)-5,6,7,8-tetrahydronaphthalene-2-carbonitrile;
5-((3-(5,6-Dihydro-4H-pyrrolo[1,2-b]pyrazol-3-yl)-1H-indazol-5-yl)amino)-5,6,7,8-tetrahydronaphthalene-2-carbonitrile;
5-((3-(1-methyl-2-oxo-1,2-dihydropyridin-4-yl)-1H-indazol-5-yl)amino)-5,6,7,8-tetrahydronaphthalene-2-carbonitrile;
5-((3-(Pyridin-3-yl)-1H-indazol-5-yl)amino)-5,6,7,8-tetrahydronaphthalene-2-carbonitrile;
(Z)-5-((3-(Prop-1-en-1-yl)-1H-indazol-5-yl)amino)-5,6,7,8-tetrahydronaphthalene-2-carbonitrile;
5-((3-(5-Methylpyridin-3-yl)-1H-indazol-5-yl)amino)-5,6,7,8-tetrahydronaphthalene-2-carbonitrile;
1-Methoxy-5-{[3-(1,3-oxazol-5-yl)-1H-indazol-5-yl]amino}-5,6,7,8-tetrahydronaphthalene-2-carbonitrile;
2-Methoxy-8-((3-(oxazol-5-yl)-1H-indazol-5-yl)amino)-5,6,7,8-tetrahydroquinoline-3-carbonitrile;
7-((3-(Oxazol-5-yl)-1H-indazol-5-yl)oxy)-6,7-dihydro-5H-cyclopenta[b]pyridine-3-carbonitrile;
5-((3-(Oxazol-5-yl)-1H-indazol-5-yl)amino)-6,7,8,9-tetrahydro-5H-benzo[7]annulene-2-carbonitrile;
3,3-Dimethyl-1-((3-(oxazol-5-yl)-1H-indazol-5-yl)amino)-2,3-dihydro-1H-indene-5-carbonitrile;
2-Methyl-8-((3-(oxazol-5-yl)-1H-indazol-5-yl)amino)-5,6,7,8-tetrahydroquinoline-3-carbonitrile;
4-Methyl-7-((3-(oxazol-5-yl)-1H-indazol-5-yl)oxy)-6,7-dihydro-5H-cyclopenta[b]pyridine-3-carbonitrile;
4-Methyl-7-((3-(oxazol-yl)-1H-indazol-5-yl)amino)-6,7-dihydro-5H-cyclopenta[b]pyridine-3-carbonitrile;
7-((3-(Oxazol-5-yl)-1H-indazol-5-yl)amino)-6,7-dihydro-5H-cyclopenta[b]pyridine-3-carbonitrile;
3-Methyl-5-((3-(oxazol-5-yl)-1H-indazol-5-yl)amino)-5,6,7,8-tetrahydronaphthalene-2-carbonitrile;
1-(Methyl(3-(oxazol-5-yl)-1H-indazol-5-yl)amino)-2,3-dihydro-1H-indene-5-carbonitrile;
2-Chloro-8-((3-(oxazol-5-yl)-1H-indazol-5-yl)amino)-5,6,7,8-tetrahydroquinoline-3-carbonitrile;
4-Methoxy-1-((3-(oxazol-5-yl)-1H-indazol-5-yl)amino)-2,3-dihydro-1H-indene-5-carbonitrile;
5-((3-(Isoxazol-4-yl)-1H-indazol-5-yl)amino)-3-methoxy-5,6,7,8-tetrahydronaphthalene-2-carbonitrile;
8,8-Dimethyl-5-((3-(oxazol-5-yl)-1H-indazol-5-yl)amino)-5,6,7,8-tetrahydronaphthalene-2-carbonitrile;
5-((3-(2-Ethylpyridin-4-yl)-1H-indazol-5-yl)amino)-5,6,7,8-tetrahydronaphthalene-2-carbonitrile;
5-((3-(2-Methoxypyridin-4-yl)-1H-indazol-5-yl)amino)-5,6,7,8-tetrahydronaphthalene-2-carbonitrile;
1-((3-(Isoxazol-4-yl)-1H-indazol-5-yl)amino)-4-methyl-2,3-dihydro-1H-indene-5-carbonitrile;
5-((3-(Oxazol-5-yl)-1H-indazol-5-yl)amino)-5,6,7,8-tetrahydronaphthalene-2-carbonitrile;
8-((3-(Oxazol-5-yl)-1H-indazol-5-yl)amino)-5,6,7,8-tetrahydroquinoline-3-carbonitrile;
8-((3-(2-Ethylpyridin-4-yl)-1H-indazol-5-yl)oxy)-5,6,7,8-tetrahydroquinoline-3-carbonitrile;
8-((3-(Oxazol-5-yl)-1H-indazol-5-yl)oxy)-5,6,7,8-tetrahydroisoquinoline-3-carbonitrile;
8-((3-(2-Methoxypyridin-4-yl)-1H-indazol-5-yl)oxy)-5,6,7,8-tetrahydroquinoline-3-carbonitrile;
1-((3-Cyclopropyl-1H-indazol-5-yl)oxy)-2,3-dihydro-1H-indene-5-carbonitrile;
8-((3-Bromo-1H-indazol-5-yl)amino)-5,6,7,8-tetrahydroquinoline-3-carbonitrile;
8-((3-Chloro-1H-indazol-5-yl)amino)-5,6,7,8-tetrahydroquinoline-3-carbonitrile;
1-((3-Iodo-1H-indazol-5-yl)amino)-4-methyl-2,3-dihydro-1H-indene-5-carbonitrile;
1-((3-Iodo-1H-indazol-5-yl)amino)-2,3-dihydro-1H-indene-5-carbonitrile;
1-((3-Methyl-1H-indazol-5-yl)amino)-2,3-dihydro-1H-indene-4-carbonitrile;
3-((3-Methyl-1H-indazol-5-yl)amino)-2,3-dihydro-1H-indene-5-carbonitrile;
7-Methyl-1-((3-(oxazol-5-yl)-1H-indazol-5-yl)amino)-2,3-dihydro-1H-indene-5-carbonitrile;
5-((5-Cyano-7-fluoro-2,3-dihydro-1H-inden-1-yl)amino)-3-(oxazol-5-yl)-1H-indazol-2-ium;
5-((3-Cyclopropyl-1H-indazol-5-yl)oxy)-5,6,7,8-tetrahydroquinoline-2-carbonitrile;
1-((3-Cyclopropyl-1H-indazol-5-yl)amino)-2,3-dihydro-1H-indene-5-carbonitrile;
5-((3-Iodo-1H-indazol-5-yl)oxy)-5,6,7,8-tetrahydroquinoline-2-carbonitrile;
8-((3-Cyclopropyl-1H-indazol-5-yl)oxy)-5,6,7,8-tetrahydroquinoline-3-carbonitrile;
8-((3-Iodo-1H-indazol-5-yl)oxy)-5,6,7,8-tetrahydroquinoline-3-carbonitrile;
3,3-Difluoro-1-((3-(oxazol-5-yl)-1H-indazol-5-yl)amino)-2,3-dihydro-1H-indene-5-carbonitrile;
5-((3-(Cyclopropylmethyl)-1H-indazol-5-yl)amino)-5,6,7,8-tetrahydronaphthalene-2-carbonitrile;
3′-((3-(Oxazol-5-yl)-1H-indazol-5-yl)amino)-2′,3′-dihydrospiro[cyclopropane-1,1′-indene]-6′-carbonitrile;
8-((3-(1-(Difluoromethyl)-1H-pyrazol-4-yl)-1H-indazol-5-yl)oxy)-5,6,7,8-tetrahydroquinoline-3-carbonitrile;
8-((3-(Thiazol-5-yl)-1H-indazol-5-yl)amino)-5,6,7,8-tetrahydroquinoline-3-carbonitrile;
1-Chloro-5-((3-(oxazol-5-yl)-1H-indazol-5-yl)amino)-5,6,7,8-tetrahydronaphthalene-2-carbonitrile;
4-Chloro-8-((3-(oxazol-5-yl)-1H-indazol-5-yl)amino)-5,6,7,8-tetrahydroquinoline-3-carbonitrile;
8-((3-(Oxazol-5-yl)-1H-indazol-5-yl)amino)-4-(trifluoromethyl)-5,6,7,8-tetrahydroquinoline-3-carbonitrile;
4-Methoxy-8-((3-(oxazol-5-yl)-1H-indazol-5-yl)amino)-5,6,7,8-tetrahydroquinoline-3-carbonitrile;
4-(Difluoromethyl)-8-((3-(oxazol-5-yl)-1H-indazol-5-yl)amino)-5,6,7,8-tetrahydroquinoline-3-carbonitrile;
8-((3-(1H-Pyrrol-2-yl)-1H-indazol-5-yl)amino)-5,6,7,8-tetrahydroquinoline-3-carbonitrile;
8-((3-(1-Methyl-1H-pyrrol-3-yl)-1H-indazol-5-yl)amino)-5,6,7,8-tetrahydroquinoline-3-carbonitrile;
4-Methyl-8-((3-(oxazol-5-yl)-1H-indazol-5-yl)amino)-5,6,7,8-tetrahydroquinoline-3-carbonitrile;
2,2,4-Trifluoro-1-((3-(oxazol-5-yl)-1H-indazol-5-yl)amino)-2,3-dihydro-1H-indene-5-carbonitrile;
5-((3-(Trifluoromethyl)-1H-indazol-5-yl)oxy)-5,6,7,8-tetrahydronaphthalene-2-carbonitrile;
5-((3-(Trifluoromethyl)-1H-indazol-5-yl)amino)-5,6,7,8-tetrahydronaphthalene-2-carbonitrile;
4-Methoxy-7-((3-(oxazol-5-yl)-1H-indazol-5-yl)amino)-6,7-dihydro-5H-cyclopenta[b]pyridine-3-carbonitrile;
2,2-Difluoro-1-((3-(2-methyloxazol-5-yl)-1H-indazol-5-yl)amino)-2,3-dihydro-1H-indene-5-carbonitrile;
4-Fluoro-1-((3-(2-methyloxazol-5-yl)-1H-indazol-5-yl)oxy)-2,3-dihydro-1H-indene-5-carbonitrile;
5-((3-(1H-Pyrazol-5-yl)-1H-indazol-5-yl)amino)-5,6,7,8-tetrahydronaphthalene-2-carbonitrile;
8-((3-(3-Methylisothiazol-5-yl)-1H-indazol-5-yl)amino)-5,6,7,8-tetrahydroquinoline-3-carbonitrile;
2-Methyl-8-((3-(2-methyloxazol-5-yl)-1H-indazol-5-yl)amino)-5,6,7,8-tetrahydroquinoline-3-carbonitrile
6-Fluoro-1-((3-(2-methyloxazol-5-yl)-1H-indazol-5-yl)oxy)-2,3-dihydro-1H-indene-5-carbonitrile;
8-((3-(Pyrazin-2-yl)-1H-indazol-5-yl)oxy)-5,6,7,8-tetrahydroquinoline-3-carbonitrile;
8-Deuterio-8-((3-(oxazol-5-yl)-1H-indazol-5-yl)amino)-5,6,7,8-tetrahydroquinoline-3-carbonitrile;
5-[[3-(1,3-Oxazol-5-yl)-1H-indazol-5-yl]oxy]-5,6,7,8-tetrahydroquinoxaline-2-carbonitrile;
2,4-Dimethyl-8-[[3-(1,3-oxazol-5-yl)-1H-indazol-5-yl]amino]-5,6,7,8-tetrahydroquinoline-3-carbonitrile;
2-Methoxy-4-methyl-8-[[3-(1,3-oxazol-5-yl)-1H-indazol-5-yl]amino]-5,6,7,8-tetrahydroquinoline-3-carbonitrile;
2-Chloro-4-methyl-7-[[3-(1,3-oxazol-5-yl)-1H-indazol-5-yl]oxy]-6,7-dihydro-5H-cyclopenta[b]pyridine-3-carbonitrile;
4,6-Difluoro-1-[[3-(1,3-oxazol-5-yl)-1H-indazol-5-yl]oxy]-2,3-dihydro-1H-indene-5-carbonitrile;
trans-3-Methyl-1-[[3-(1,3-oxazol-5-yl)-1H-indazol-5-yl]amino]-2,3-dihydro-1H-indene-5-carbonitrile;
cis-3-Methyl-1-[[3-(1,3-oxazol-5-yl)-1H-indazol-5-yl]amino]-2,3-dihydro-1H-indene-5-carbonitrile;
2,4-Dimethyl-7-[[3-(1,3-oxazol-5-yl)-1H-indazol-5-yl]amino]-6,7-dihydro-5H-cyclopenta[b]pyridine-3-carbonitrile;
2-Chloro-7-[[3-(1,3-oxazol-5-yl)-1H-indazol-5-yl]oxy]-6,7-dihydro-5H-cyclopenta[b]pyridine-3-carbonitrile;
6,6-Dimethyl-7-[[3-(1,3-oxazol-5-yl)-1H-indazol-5-yl]amino]-5,7-dihydrocyclopenta[b]pyridine-3-carbonitrile;
cis-6-Methyl-5-((3-(oxazol-5-yl)-1H-indazol-5-yl)amino)-5,6,7,8-tetrahydronaphthalene-2-carbonitrile;
trans-6-Methyl-5-((3-(oxazol-5-yl)-1H-indazol-5-yl)amino)-5,6,7,8-tetrahydronaphthalene-2-carbonitrile;
cis-6-Methyl-5-[[3-(1,3-oxazol-5-yl)-1H-indazol-5-yl]oxy]-5,6,7,8-tetrahydronaphthalene-2-carbonitrile;
trans-6-Methyl-5-[[3-(1,3-oxazol-5-yl)-1H-indazol-5-yl]oxy]-5,6,7,8-tetrahydronaphthalene-2-carbonitrile;
2-Chloro-8-((3-(oxazol-5-yl)-1H-indazol-5-yl)oxy)-5,6,7,8-tetrahydroquinoline-3-carbonitrile;
2-Chloro-8-[[3-(1,2-oxazol-4-yl)-1H-indazol-5-yl]oxy]-5,6,7,8-tetrahydroquinoline-3-carbonitrile;
2-Methoxy-8-[[3-(1,3-oxazol-5-yl)-1H-indazol-5-yl]oxy]-5,6,7,8-tetrahydroquinoline-3-carbonitrile;
3-Fluoro-5-[[3-(1,3-oxazol-5-yl)-1H-indazol-5-yl]oxy]-5,6,7,8-tetrahydronaphthalene-2-carbonitrile;
2-Chloro-8-[(3-cyclopropyl-1H-indazol-5-yl)oxy]-5,6,7,8-tetrahydroquinoline-3-carbonitrile;
5-((3-(Oxazol-5-yl)-1H-indazol-5-yl)oxy)-6,7-dihydro-5H-cyclopenta[b]pyrazine-2-carbonitrile;
2-Chloro-8-[(3-cyclopropyl-1H-indazol-5-yl)amino]-4-methyl-5,6,7,8-tetrahydroquinoline-3-carbonitrile;
2-Chloro-8-[(3-cyclopropyl-1H-indazol-5-yl)oxy]-4-methyl-5,6,7,8-tetrahydroquinoline-3-carbonitrile;
2-Chloro-7-[(3-methoxy-1H-indazol-5-yl)oxy]-6,7-dihydro-5H-cyclopenta[b]pyridine-3-carbonitrile;
2-Chloro-8-[(3-methoxy-1H-indazol-5-yl)oxy]-5,6,7,8-tetrahydroquinoline-3-carbonitrile;
2-Bromo-7-[(3-methoxy-1H-indazol-5-yl)oxy]-6,7-dihydro-5H-cyclopenta[b]pyridine-3-carbonitrile;
2-Bromo-8-[(3-methoxy-1H-indazol-5-yl)oxy]-5,6,7,8-tetrahydroquinoline-3-carbonitrile;
6-Chloro-1-[(3-methoxy-1H-indazol-5-yl)oxy]-2,3-dihydro-1H-indene-5-carbonitrile;
8-[[6-Methyl-3-(1,3-oxazol-5-yl)-1H-indazol-5-yl]amino]-5,6,7,8-tetrahydroquinoline-3-carbonitrile;
4-((3-(Oxazol-5-yl)-1H-indazol-5-yl)amino)chromane-7-carbonitrile;
4-((3-(Isoxazol-4-yl)-1H-indazol-5-yl)amino)chromane-7-carbonitrile;
4-((3-Iodo-1H-indazol-5-yl)amino)chromane-7-carbonitrile; and
4-((3-(Oxazol-5-yl)-1H-indazol-5-yl)amino)-3,4-dihydro-2H-pyrano[3,2-b]pyridine-7-carbonitrile,

or a pharmaceutically acceptable salt of any of the aforementioned.

It is further appreciated that certain features of the invention, which are, for clarity, described in the context of separate embodiments, can also be provided in combination in a single embodiment (while the embodiments are intended to be combined as if written in multiply dependent form). Conversely, various features of the invention which are, for brevity, described in the context of a single embodiment, can also be provided separately or in any suitable subcombination. Thus, it is contemplated as features described as embodiments of the compounds of Formula I can be combined in any suitable combination.

At various places in the present specification, certain features of the compounds are disclosed in groups or in ranges. It is specifically intended that such a disclosure include each and every individual subcombination of the members of such groups and ranges. For example, the term “C₁₋₆ alkyl” is specifically intended to individually disclose (without limitation) methyl, ethyl, C₃ alkyl, C₄ alkyl, C₅ alkyl and C₆ alkyl.

The term “n-membered,” where n is an integer, typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring and 1,2,3,4-tetrahydro-naphthalene is an example of a 10-membered cycloalkyl group.

At various places in the present specification, variables defining divalent linking groups may be described. It is specifically intended that each linking substituent include both the forward and backward forms of the linking substituent. For example, —NR(CR′R″)ₙ— includes both —NR(CR′R″)ₙ— and —(CR′R″)ₙNR— and is intended to disclose each of the forms individually. Where the structure requires a linking group, the Markush variables listed for that group are understood to be linking groups. For example, if the structure requires a linking group and the Markush group definition for that variable lists “alkyl” or “aryl” then it is understood that the “alkyl” or “aryl” represents a linking alkylene group or arylene group, respectively.

The term “substituted” means that an atom or group of atoms formally replaces hydrogen as a “substituent” attached to another group. The term “substituted”, unless otherwise indicated, refers to any level of substitution, e.g., mono-, di-, tri-, tetra- or penta-substitution, where such substitution is permitted. The substituents are independently selected, and substitution may be at any chemically accessible position. It is to be understood that substitution at a given atom is limited by valency. It is to be understood that substitution at a given atom results in a chemically stable molecule. The phrase “optionally substituted” means unsubstituted or substituted. The term “substituted” means that a hydrogen atom is removed and replaced by a substituent. A single divalent substituent, e.g., oxo, can replace two hydrogen atoms.

The term “Cₙ₋ₘ” indicates a range which includes the endpoints, wherein n and m are integers and indicate the number of carbons. Examples include C₁₋₄, C₁₋₆ and the like.

The term “alkyl” employed alone or in combination with other terms, refers to a saturated hydrocarbon group that may be straight-chained or branched. The term “Cₙ₋ₘ alkyl”, refers to an alkyl group having n to m carbon atoms. An alkyl group formally corresponds to an alkane with one C—H bond replaced by the point of attachment of the alkyl group to the remainder of the compound. In some embodiments, the alkyl group contains from 1 to 6 carbon atoms, from 1 to 4 carbon atoms, from 1 to 3 carbon atoms, or 1 to 2 carbon atoms. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl and the like.

The term “alkenyl” employed alone or in combination with other terms, refers to a straight-chain or branched hydrocarbon group corresponding to an alkyl group having one or more double carbon-carbon bonds. An alkenyl group formally corresponds to an alkene with one C—H bond replaced by the point of attachment of the alkenyl group to the remainder of the compound. The term “Cₙ₋ₘ alkenyl” refers to an alkenyl group having n to m carbons. In some embodiments, the alkenyl moiety contains 2 to 6, 2 to 4, or 2 to 3 carbon atoms. Example alkenyl groups include, but are not limited to, ethenyl, n-propenyl, isopropenyl, n-butenyl, sec-butenyl and the like.

The term “alkynyl” employed alone or in combination with other terms, refers to a straight-chain or branched hydrocarbon group corresponding to an alkyl group having one or more triple carbon-carbon bonds. An alkynyl group formally corresponds to an alkyne with one C—H bond replaced by the point of attachment of the alkyl group to the remainder of the compound. The term “Cₙ₋ₘ alkynyl” refers to an alkynyl group having n to m carbons.

Example alkynyl groups include, but are not limited to, ethynyl, propyn-1-yl, propyn-2-yl and the like. In some embodiments, the alkynyl moiety contains 2 to 6, 2 to 4, or 2 to 3 carbon atoms.

The term “alkylene”, employed alone or in combination with other terms, refers to a divalent alkyl linking group. An alkylene group formally corresponds to an alkane with two C—H bond replaced by points of attachment of the alkylene group to the remainder of the compound. The term “Cₙ₋ₘ alkylene” refers to an alkylene group having n to m carbon atoms. Examples of alkylene groups include, but are not limited to, ethan-1,2-diyl, ethan-1,1-diyl, propan-1,3-diyl, propan-1,2-diyl, propan-1,1-diyl, butan-1,4-diyl, butan-1,3-diyl, butan-1,2-diyl, 2-methyl-propan-1,3-diyl and the like.

The term “alkoxy”, employed alone or in combination with other terms, refers to a group of formula —O-alkyl, wherein the alkyl group is as defined above. The term “Cₙ₋ₘ alkoxy” refers to an alkoxy group, the alkyl group of which has n to m carbons. Example alkoxy groups include methoxy, ethoxy, propoxy (e.g., n-propoxy and isopropoxy), t-butoxy and the like. In some embodiments, the alkyl group has 1 to 6, 1 to 4, or 1 to 3 carbon atoms.

The term “C n-m dialkoxy” refers to a linking group of formula —O—(Cₙ₋ₘ alkyl)-O—, the alkyl group of which has n to m carbons. Example dialkyoxy groups include —OCH₂CH₂O— and OCH₂CH₂CH₂O—. In some embodiments, the two O atoms of a C n-m dialkoxy group may be attached to the same B atom to form a 5- or 6-membered heterocycloalkyl group.

The term “amino” refers to a group of formula —NH₂.

The term “carbonyl”, employed alone or in combination with other terms, refers to a —C(═O)— group, which also may be written as C(O).

The term “cyano” or “nitrile” refers to a group of formula —C≡N, which also may be written as —CN.

The terms “halo” or “halogen”, used alone or in combination with other terms, refers to fluoro, chloro, bromo and iodo. In some embodiments, “halo” refers to a halogen atom selected from F, Cl, or Br. In some embodiments, halo groups are F.

The term “haloalkyl” as used herein refers to an alkyl group in which one or more of the hydrogen atoms has been replaced by a halogen atom. The term “Cₙ₋ₘ haloalkyl” refers to a Cₙ₋ₘ alkyl group having n to m carbon atoms and from at least one up to {2(n to m)+1}halogen atoms, which may either be the same or different. In some embodiments, the halogen atoms are fluoro atoms. In some embodiments, the haloalkyl group has 1 to 6 or 1 to 4 carbon atoms. Example haloalkyl groups include CF₃, C₂F₅, CHF₂, CH₂F, CCl₃, CHCl₂, C₂Cl₅ and the like. In some embodiments, the haloalkyl group is a fluoroalkyl group.

The term “haloalkoxy”, employed alone or in combination with other terms, refers to a group of formula —O-haloalkyl, wherein the haloalkyl group is as defined above. The term “Cₙ₋ₘ haloalkoxy” refers to a haloalkoxy group, the haloalkyl group of which has n to m carbons. Example haloalkoxy groups include trifluoromethoxy and the like. In some embodiments, the haloalkoxy group has 1 to 6, 1 to 4, or 1 to 3 carbon atoms.

The term “oxo” refers to an oxygen atom as a divalent substituent, forming a carbonyl group when attached to carbon, or attached to a heteroatom forming a sulfoxide or sulfone group, or an N-oxide group. In some embodiments, heterocyclic groups may be optionally substituted by 1 or 2 oxo (═O) substituents.

The term “sulfido” refers to a sulfur atom as a divalent substituent, forming a thiocarbonyl group (C═S) when attached to carbon.

The term “oxidized” in reference to a ring-forming N atom refers to a ring-forming N-oxide.

The term “oxidized” in reference to a ring-forming S atom refers to a ring-forming sulfonyl or ring-forming sulfinyl.

The term “aromatic” refers to a carbocycle or heterocycle having one or more polyunsaturated rings having aromatic character (i.e., having (4n+2) delocalized π (pi) electrons where n is an integer).

The term “aryl,” employed alone or in combination with other terms, refers to an aromatic hydrocarbon group, which may be monocyclic or polycyclic (e.g., having 2 fused rings). The term “Cₙ₋ₘ aryl” refers to an aryl group having from n to m ring carbon atoms.

Aryl groups include, e.g., phenyl, naphthyl, and the like. In some embodiments, aryl groups have from 6 to about 10 carbon atoms. In some embodiments aryl groups have 6 carbon atoms. In some embodiments aryl groups have 10 carbon atoms. In some embodiments, the aryl group is phenyl.

The term “heteroaryl” or “heteroaromatic,” employed alone or in combination with other terms, refers to a monocyclic or polycyclic aromatic heterocycle having at least one heteroatom ring member selected from sulfur, oxygen and nitrogen. In some embodiments, the heteroaryl ring has 1, 2, 3 or 4 heteroatom ring members independently selected from nitrogen, sulfur and oxygen. In some embodiments, any ring-forming N in a heteroaryl moiety can be an N-oxide. In some embodiments, the heteroaryl has 5-14 ring atoms including carbon atoms and 1, 2, 3 or 4 heteroatom ring members independently selected from nitrogen, sulfur and oxygen. In some embodiments, the heteroaryl has 5-10 ring atoms including carbon atoms and 1, 2, 3 or 4 heteroatom ring members independently selected from nitrogen, sulfur and oxygen. In some embodiments, the heteroaryl has 5-6 ring atoms and 1 or 2 heteroatom ring members independently selected from nitrogen, sulfur and oxygen. In some embodiments, the heteroaryl is a five-membered or six-membered heteroaryl ring. In other embodiments, the heteroaryl is an eight-membered, nine-membered or ten-membered fused bicyclic heteroaryl ring. Example heteroaryl groups include, but are not limited to, pyridinyl (pyridyl), pyrimidinyl, pyrazinyl, pyridazinyl, and the like.

A five-membered heteroaryl ring is a heteroaryl group having five ring atoms wherein one or more (e.g., 1, 2 or 3) ring atoms are independently selected from N, O and S. Exemplary five-membered ring heteroaryls include thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl and 1,3,4-oxadiazolyl.

A six-membered heteroaryl ring is a heteroaryl group having six ring atoms wherein one or more (e.g., 1, 2 or 3) ring atoms are independently selected from N, O and S. Exemplary six-membered ring heteroaryls are pyridyl, pyrazinyl, pyrimidinyl, triazinyl, isoindolyl, and pyridazinyl.

The term “cycloalkyl,” employed alone or in combination with other terms, refers to a non-aromatic hydrocarbon ring system (monocyclic, bicyclic or polycyclic), including cyclized alkyl and alkenyl groups. The term “Cₙ₋ₘ cycloalkyl” refers to a cycloalkyl that has n to m ring member carbon atoms. Cycloalkyl groups can include mono- or polycyclic (e.g., having 2, 3 or 4 fused rings) groups and spirocycles. Cycloalkyl groups can have 3, 4, 5, 6 or 7 ring-forming carbons (C₃₋₇). In some embodiments, the cycloalkyl group has 3 to 6 ring members, 3 to 5 ring members, or 3 to 4 ring members. In some embodiments, the cycloalkyl group is monocyclic. In some embodiments, the cycloalkyl group is monocyclic or bicyclic. In some embodiments, the cycloalkyl group is a C₃₋₆ monocyclic cycloalkyl group. Ring-forming carbon atoms of a cycloalkyl group can be optionally oxidized to form an oxo or sulfido group. Cycloalkyl groups also include cycloalkylidenes. In some embodiments, cycloalkyl is cyclopropyl, cyclobutyl, cyclopentyl or cyclohexyl. Also included in the definition of cycloalkyl are moieties that have one or more aromatic rings fused (i.e., having a bond in common with) to the cycloalkyl ring, e.g., benzo or thienyl derivatives of cyclopentane, cyclohexane and the like. A cycloalkyl group containing a fused aromatic ring can be attached through any ring-forming atom including a ring-forming atom of the fused aromatic ring. Examples of cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, bicyclo[1.1.1]pentanyl, bicyclo[2.1.1]hexanyl, and the like. In some embodiments, the cycloalkyl group is cyclopropyl, cyclobutyl, cyclopentyl, or cyclohexyl.

The term “heterocycloalkyl,” employed alone or in combination with other terms, refers to a non-aromatic ring or ring system, which may optionally contain one or more alkenylene groups as part of the ring structure, which has at least one heteroatom ring member independently selected from nitrogen, sulfur, oxygen and phosphorus, and which has 4-10 ring members, 4-7 ring members, or 4-6 ring members. Included within the term “heterocycloalkyl” are monocyclic 4-, 5-, 6- and 7-membered heterocycloalkyl groups. Heterocycloalkyl groups can include mono- or bicyclic (e.g., having two fused or bridged rings) or spirocyclic ring systems. In some embodiments, the heterocycloalkyl group is a monocyclic group having 1, 2 or 3 heteroatoms independently selected from nitrogen, sulfur and oxygen. Ring-forming carbon atoms and heteroatoms of a heterocycloalkyl group can be optionally oxidized to form an oxo or sulfido group or other oxidized linkage (e.g., C(O), S(O), C(S) or S(O)₂, N-oxide etc.) or a nitrogen atom can be quaternized. The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. Also included in the definition of heterocycloalkyl are moieties that have one or more aromatic rings fused (i.e., having a bond in common with) to the heterocycloalkyl ring, e.g., benzo or thienyl derivatives of piperidine, morpholine, azepine, etc. A heterocycloalkyl group containing a fused aromatic ring can be attached through any ring-forming atom including a ring-forming atom of the fused aromatic ring.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas an azetidin-3-yl ring is attached at the 3-position.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds and the like can also be present in the compounds described herein, and all such stable isomers are contemplated in the present invention. Cis and trans geometric isomers of the compounds of the present invention are described and may be isolated as a mixture of isomers or as separated isomeric forms.

Resolution of racemic mixtures of compounds can be carried out by any of numerous methods known in the art. One method includes fractional recrystallization using a chiral resolving acid which is an optically active, salt-forming organic acid. Suitable resolving agents for fractional recrystallization methods are, e.g., optically active acids, such as the D and L forms of tartaric acid, diacetyltartaric acid, dibenzoyltartaric acid, mandelic acid, malic acid, lactic acid or the various optically active camphorsulfonic acids such as 3-camphorsulfonic acid. Other resolving agents suitable for fractional crystallization methods include stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane and the like.

Resolution of racemic mixtures can also be carried out by elution on a column packed with an optically active resolving agent (e.g., dinitrobenzoylphenylglycine). Suitable elution solvent composition can be determined by one skilled in the art.

In some embodiments, the compounds of the invention have the (R)-configuration. In other embodiments, the compounds have the (S)-configuration. In compounds with more than one chiral centers, each of the chiral centers in the compound may be independently (R) or (S), unless otherwise indicated.

Compounds of the invention also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone—enol pairs, amide—imidic acid pairs, lactam—lactim pairs, enamine—imine pairs, and annular forms where a proton can occupy two or more positions of a heterocyclic system, e.g., 1H- and 3H-imidazole, 1H-, 2H- and 4H-1,2,4-triazole, 1H- and 2H-isoindole and 1H- and 2H-pyrazole. Tautomeric forms can be in equilibrium or sterically locked into one form by appropriate substitution.

Compounds of the invention can also include all isotopes of atoms occurring in the intermediates or final compounds. Isotopes include those atoms having the same atomic number but different mass numbers. For example, isotopes of hydrogen include tritium and deuterium. One or more constituent atoms of the compounds of the invention can be replaced or substituted with isotopes of the atoms in natural or non-natural abundance. In some embodiments, the compound includes at least one deuterium atom. For example, one or more hydrogen atoms in a compound of the present disclosure can be replaced or substituted by deuterium. In some embodiments, the compound includes two or more deuterium atoms. In some embodiments, the compound includes 1, 2, 3, 4, 5, 6, 7, 8, 9, 10, 11 or 12 deuterium atoms. Synthetic methods for including isotopes into organic compounds are known in the art (Deuterium Labeling in Organic Chemistry by Alan F. Thomas (New York, N.Y., Appleton-Century-Crofts, 1971; The Renaissance of H/D Exchange by Jens Atzrodt, Volker Derdau, Thorsten Fey and Jochen Zimmermann, Angew. Chem. Int. Ed. 2007, 7744-7765; The Organic Chemistry of Isotopic Labelling by James R. Hanson, Royal Society of Chemistry, 2011). Isotopically labeled compounds can used in various studies such as NMR spectroscopy, metabolism experiments, and/or assays.

The term, “compound,” as used herein is meant to include all stereoisomers, geometric isomers, tautomers and isotopes of the structures depicted. The term is also meant to refer to compounds of the inventions, regardless of how they are prepared, e.g., synthetically, through biological process (e.g., metabolism or enzyme conversion), or a combination thereof.

All compounds, and pharmaceutically acceptable salts thereof, can be found together with other substances such as water and solvents (e.g., hydrates and solvates) or can be isolated. When in the solid state, the compounds described herein and salts thereof may occur in various forms and may, e.g., take the form of solvates, including hydrates. The compounds may be in any solid state form, such as a polymorph or solvate, so unless clearly indicated otherwise, reference in the specification to compounds and salts thereof should be understood as encompassing any solid state form of the compound.

In some embodiments, the compounds of the invention, or salts thereof, are substantially isolated. By “substantially isolated” is meant that the compound is at least partially or substantially separated from the environment in which it was formed or detected. Partial separation can include, e.g., a composition enriched in the compounds of the invention. Substantial separation can include compositions containing at least about 50%, at least about 60%, at least about 70%, at least about 80%, at least about 90%, at least about 95%, at least about 97%, or at least about 99% by weight of the compounds of the invention, or salt thereof.

The phrase “pharmaceutically acceptable” is employed herein to refer to those compounds, materials, compositions and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The expressions, “ambient temperature” and “room temperature,” as used herein, are understood in the art, and refer generally to a temperature, e.g., a reaction temperature, that is about the temperature of the room in which the reaction is carried out, e.g., a temperature from about 20° C. to about 30° C.

The present invention also includes pharmaceutically acceptable salts of the compounds described herein. The term “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the non-toxic salts of the parent compound formed, e.g., from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol or butanol) or acetonitrile (MeCN) are preferred. Lists of suitable salts are found in Remington's Pharmaceutical Sciences, 17^{th} Ed., (Mack Publishing Company, Easton, 1985), p. 1418, Berge et al., J. Pharm. Sci., 1977, 66(1), 1-19 and in Stahl et al., Handbook of Pharmaceutical Salts: Properties, Selection, and Use, (Wiley, 2002). In some embodiments, the compounds described herein include the N-oxide forms.

### Synthesis

Compounds of the invention, including salts thereof, can be prepared using known organic synthesis techniques and can be synthesized according to any of numerous possible synthetic routes, such as those in the Schemes below.

The reactions for preparing compounds of the invention can be carried out in suitable solvents which can be readily selected by one of skill in the art of organic synthesis. Suitable solvents can be substantially non-reactive with the starting materials (reactants), the intermediates or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of compounds of the invention can involve the protection and deprotection of various chemical groups. The need for protection and deprotection, and the selection of appropriate protecting groups, can be readily determined by one skilled in the art. The chemistry of protecting groups is described, e.g., in Kocienski, Protecting Groups, (Thieme, 2007); Robertson, Protecting Group Chemistry, (Oxford University Press, 2000); Smith et al., March's Advanced Organic Chemistry: Reactions, Mechanisms, and Structure, 6^{th} Ed. (Wiley, 2007); Peturssion et al., “Protecting Groups in Carbohydrate Chemistry,” J Chem. Educ., 1997, 74(11), 1297; and Wuts et al., Protective Groups in Organic Synthesis, 4th Ed., (Wiley, 2006).

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry or by chromatographic methods such as high performance liquid chromatography (HPLC) or thin layer chromatography (TLC).

The Schemes below provide general guidance in connection with preparing the compounds of the invention. One skilled in the art would understand that the preparations shown in the Schemes can be modified or optimized using general knowledge of organic chemistry to prepare various compounds of the invention.

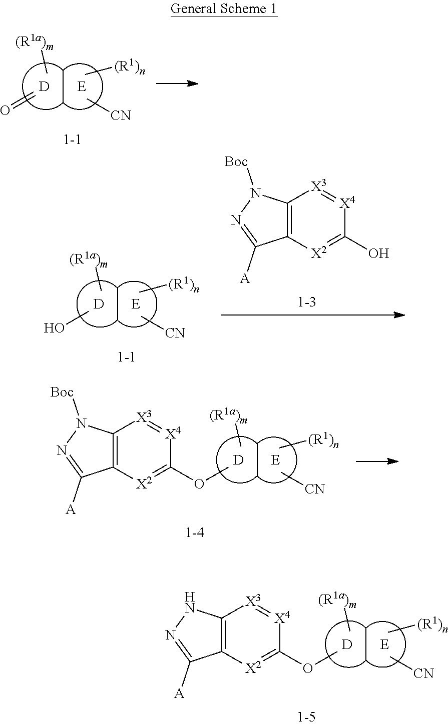

A general synthesis of compounds of the invention comprises a procedure as shown in General Scheme 1 above. Ketone 1-1 can be reduced with a hydride reducing agent (e.g., NaBH₄, or sodium triacetoxyborohydride) to provide alcohol 1-2. Combining alcohol 1-2 and alcohol 1-3 with a suitable activating agent (e.g., triphenylphosphine and diethyl azodicarboxylate) will lead to products of type 1-4. Deprotection with an acid (e.g., HCl or TFA) will lead to product 1-5. Products of 1-5 can be purified by silica gel chromatography, preparative reverse-phase HPLC, SFC, chiral phase HPLC, as well as other purification methods such as crystallization.

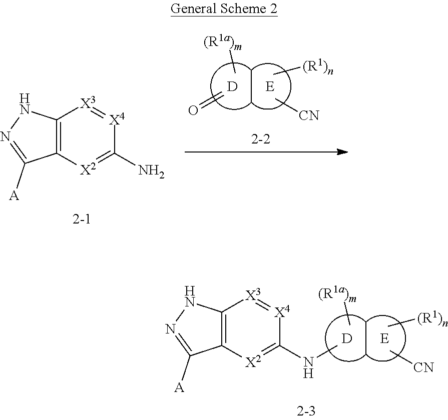

Products of type 2-3 may be prepared using the procedure as shown in General Scheme 2. A 5-aminoindazole or aza derivative thereof (2-1) is coupled with a compound 2-2 via reductive amination (e.g., in the presence of NaBH₄) to provide an amine of type 2-3. Products of type 2-3 can be purified by silica gel chromatography, preparative reverse-phase HPLC, SFC, chiral phase HPLC, as well as other purification methods such as crystallization.

### Methods of Use

Over-activation of LRRK2 kinase activity, e.g., in kinase mutant G2019S, is a mechanism in alpha-synuclein related neurodegeneration, and is implicated in diseases that are characterized by the formation of Lewy bodies. Compounds as described herein, e.g., compounds of Formula I, exhibit inhibitory activity against LRRK2 kinase, including LRRK2 mutant kinase, such as mutant G2019S. Kinase activity can be determined using a kinase assay, which typically employs a kinase substrate and a phosphate group donor, such as ATP (or a derivative thereof). An exemplary kinase assay is described in Example A.

The present disclosure provides methods of modulating (e.g., inhibiting) LRRK2 activity, by contacting LRRK2 with a compound of the invention, or a pharmaceutically acceptable salt thereof. In some embodiments, the contacting can be administering to a patient, in need thereof, a compound provided herein, or a pharmaceutically acceptable salt thereof. In certain embodiments, the compounds of the present disclosure, or pharmaceutically acceptable salts thereof, are useful for therapeutic administration to treat neurodegenerative disease. For example, a method of treating a disease or disorder associated with inhibition of LRRK2 interaction can include administering to a patient in need thereof a therapeutically effective amount of a compound provided herein, or a pharmaceutically acceptable salt thereof. The compounds of the present disclosure can be used alone, in combination with other agents or therapies or as an adjuvant or neoadjuvant for the treatment of diseases or disorders, including neurodegenerative diseases. For the uses described herein, any of the compounds of the disclosure, including any of the embodiments thereof, may be used.

Compounds and compositions as described herein, e.g., compounds of Formula I are useful in the treatment and/or prevention of LRRK2 kinase mediated disorders, including LRRK2 kinase mutant mediated diseases. LRRK2 kinase mutant G2019S mediated diseases include, but are not limited to, neurological diseases such as Parkinson's disease and other Lewy body diseases such as Parkinson disease with dementia, Parkinson's associated risk syndrome, dementia with Lewy bodies (e.g., diffuse Lewy body disease (DLBD), Lewy body dementia, Lewy body disease, cortical Lewy body disease or senile dementia of Lewy type), Lewy body variant of Alzheimer's disease (i.e., diffuse Lewy body type of Alzheimer's disease), combined Parkinson's disease and Alzheimer's disease, as well as diseases associated with glial cortical inclusions, such as syndromes identified as multiple system atrophy, including striatonigral degeneration, olivopontocerebellar atrophy, and Shy-Drager syndrome, or other diseases associated with Parkinsonism, such as Hallervorden-Spatz syndrome (also referred to as Hallervorden-Spatz disease), fronto-temporal dementia, Sandhoff disease, progressive supranuclear palsy, corticobasal degeneration, autonomic dysfunctions (e.g., postural or orthostatic hypotension), cerebellar dysfunctions, ataxia, movement disorders, cognitive deterioration, sleep disorders, hearing disorders, tremors, rigidity (e.g., joint stiffness, increased muscle tone), bradykinesia, akinesia and postural instability (failure of postural reflexes, along other disease related factors such as orthostatic hypotension or cognitive and sensory changes, which lead to impaired balance and falls); cancers, including melanoma, acute myelogenous leukemia, breast carcinoma, lung adenocarincoma, prostate adenocarcinoma, renal cell carcinoma, and papillary thyroid carcinoma; autoimmune diseases such as Inflammatory Bowel Disease (e.g. Crohn's disease and ulcerative colitis); and leprosy.

In some embodiments, a method of treating a disease is provided comprising administering to a patient in need thereof a therapeutically effective amount of a compound of Formula I, or a pharmaceutically acceptable salt thereof, wherein the disease is selected from the group consisting of Parkinson's disease, Parkinson disease with dementia, Parkinson's associated risk syndrome, dementia with Lewy bodies, Lewy body variant of Alzheimer's disease, combined Parkinson's disease and Alzheimer's disease, multiple system atrophy, striatonigral degeneration, olivopontocerebellar atrophy, Shy-Drager syndrome, Hallervorden-Spatz syndrome, fronto-temporal dementia, Sandhoff disease, progressive supranuclear palsy, corticobasal degeneration, postural hypotension, orthostatic hypotension, cerebellar dysfunctions, ataxia, movement disorders, cognitive deterioration, sleep disorders, hearing disorders, tremors, rigidity, bradykinesia, akinesia, postural instability, melanoma, acute myelogenous leukemia, breast carcinoma, lung adenocarincoma, prostate adenocarcinoma, renal cell carcinoma, papillary thyroid carcinoma, Crohn's disease, ulcerative colitis, and leprosy.

In some embodiments, a method of treating a neurological disease is provided comprising administering to a patient in need thereof a therapeutically effective amount of a compound of Formula I, or a pharmaceutically acceptable salt thereof, wherein the neurological disease is selected from the group consisting of Parkinson's disease, Parkinson disease with dementia, Parkinson's associated risk syndrome, dementia with Lewy bodies, Lewy body variant of Alzheimer's disease, combined Parkinson's disease and Alzheimer's disease, multiple system atrophy, striatonigral degeneration, olivopontocerebellar atrophy, Shy-Drager syndrome, Hallervorden-Spatz syndrome, fronto-temporal dementia, Sandhoff disease, progressive supranuclear palsy, corticobasal degeneration, postural hypotension, orthostatic hypotension, cerebellar dysfunctions, ataxia, movement disorders, cognitive deterioration, sleep disorders, hearing disorders, tremors, rigidity, bradykinesia, akinesia, and postural instability.

In some embodiments, a method of treating a neurological disease is provided comprising administering to a patient in need thereof a therapeutically effective amount of a compound of Formula I, or a pharmaceutically salt thereof, wherein the neurological disease is selected from the group consisting of Parkinson's disease, Parkinson disease with dementia, Parkinson's associated risksyndrome, dementia with Lewy bodies, Lewy body variant of Alzheimer's disease, combined Parkinson's disease and Alzheimer's disease, multiple system atrophy, striatonigral degeneration, olivopontocerebellar atrophy, and Shy-Drager syndrome.

In some embodiments, a method of treating Parkinson's disease is provided comprising administering to a patient in need thereof a therapeutically effective amount of a compound of Formula I, or a pharmaceutically acceptable salt thereof.

In some embodiments, a method of treating a cancer is provided comprising administering to a patient in need thereof a therapeutically effective amount of a compound of Formula I, or a pharmaceutically acceptable salt thereof, wherein the cancer is selected from melanoma, acute myelogenous leukemia, breast carcinoma, lung adenocarincoma, prostate adenocarcinoma, renal cell carcinoma, and papillary thyroid carcinoma.

In some embodiments, a method of treating an autoimmune disease is provided comprising administering to a patient in need thereof a therapeutically effective amount of a compound of Formula I, or a pharmaceutically acceptable salt thereof, wherein the autoimmune disease is selected from Crohn's disease and ulcerative colitis.

In some embodiments, a method of treating leprosy is provided comprising administering to a patient in need thereof a therapeutically effective amount of a compound of Formula I, or a pharmaceutically acceptable salt thereof, or a composition comprising such compound or salt thereof.

In some embodiments, the compounds as described herein, e.g., compounds of Formula I, are inhibitors of LRRK2 kinase activity. In some embodiments, the compounds as described herein, e.g. compounds of Formula I, are inhibitors of LRRK2 mutant kinase activity. In some embodiments, the compounds as described herein, e.g. compounds of Formula I, are inhibitors of LRRK2 mutant G2019S kinase activity.

Compounds as described herein, e.g., compounds of Formula I, exhibit cellular biological activities, including but not limited to reduction in phosphorylation of ser910 or ser935 in HEK-293 cells transfected with either wild-type LRRK2 or LRRK2 G2019S mutant.

In some embodiments, compounds of Formula I are selective LRRK2 G2019S mutant inhibitors as compared to wild-type LRRK2.

As used herein, the term “contacting” refers to the bringing together of the indicated moieties in an in vitro system or an in vivo system such that they are in sufficient physical proximity to interact.

The terms “individual” or “patient,” used interchangeably, refer to any animal, including mammals, preferably mice, rats, other rodents, rabbits, dogs, cats, swine, cattle, sheep, horses, or primates, and most preferably humans.

The phrase “therapeutically effective amount” refers to the amount of active compound or pharmaceutical agent that elicits the biological or medicinal response in a tissue, system, animal, individual or human that is being sought by a researcher, veterinarian, medical doctor or other clinician.

As used herein, the term “treating” or “treatment” refers to one or more of (1) inhibiting the disease; e.g., inhibiting a disease, condition or disorder in an individual who is experiencing or displaying the pathology or symptomatology of the disease, condition or disorder (i.e., arresting further development of the pathology and/or symptomatology); and (2) ameliorating the disease; e.g., ameliorating a disease, condition or disorder in an individual who is experiencing or displaying the pathology or symptomatology of the disease, condition or disorder (i.e., reversing the pathology and/or symptomatology) such as decreasing the severity of disease.

As used herein, the term “selective” or “selectivity” as it relates to kinase activity, means that a compound as described herein, e.g. a compound of Formula I, is a more potent inhibitor of a particular kinase, such as LRRK2 kinase, when compared to another kinase.

While LRRK2 has other enzymatic activities, it is understood that when inhibitory activity or selectivity of LRRK2, or any mutation thereof, is mentioned, it is the LRRK2 kinase activity that is being referred to, unless clearly stated otherwise. As such, selectivity of LRRK2 relative to another kinase indicates a comparison of the IC₅₀ of a compound on the kinase activity of LRRK2 to the IC₅₀ of the compound on the kinase activity of another kinase. For example, a compound that is 10 fold selective for LRRK2 kinase activity relative to another kinase activity will have a ratio of IC₅₀(other kinase)÷IC₅₀(LRRK2)=10 (or a ratio of IC₅₀(LRRK2)÷IC₅₀(other kinase)=0.1).

In some embodiments, a compound as described herein, e.g., a compound of Formula I, is selective for a LRRK2 mutant over wild type LRRK2. Selectivity of LRRK2 mutants relative to wild type LRRK2 indicates a comparison of the IC₅₀ of a compound on the kinase activity of the mutant LRRK2 to the IC₅₀ of the compound on the kinase activity of wild type LRRK2. For example, a compound that is 10 fold selective for LRRK2 mutant kinase activity relative to wild type LRKK2 kinase activity will have a ratio of IC₅₀(wild type LRRK2)÷IC₅₀(mutant LRRK2)=10. In some embodiments, a compound provided herein is greater than 1 fold selective, greater than 2 fold selective, greater than 5 fold selective, greater than 10 fold selective, greater than 25 fold selective, or greater than 50 fold selective for LRRK2 mutant kinase over wild type LRRK2. In some embodiments, the LRRK2 mutant is LRRK2 G2019S.

The term “LRRK2-mediated condition”, “Leucine-rich repeat kinase 2 mediated disorder” or any other variation thereof, as used herein means any disease or other condition in which LRRK2, including any mutations thereof, is known to play a role, or a disease state that is associated with elevated activity or expression of LRRK2, including any mutations thereof. For example, a “LRRK2-mediated condition” may be relieved by inhibiting LRRK2 kinase activity. Such conditions include certain neurodegenerative diseases, such as Lewy body diseases, including, but not limited to, Parkinson's disease, Lewy body variant of Alzheimer's disease, combined Parkinson's disease and Alzheimer's disease, dementia with Lewy bodies, diffuse Lewy body disease, as well as any syndrome identified as multiple system atrophy; certain cancers, such as melanoma, papillary renal cell carcinoma and papillary thyroid carcinoma; certain autoimmune diseases, such as Inflammatory Bowel Disease (e.g. Crohn's disease and ulcerative colitis); and leprosy.

The term “neurodegenerative diseases” includes any disease or condition characterized by problems with movements, such as ataxia, and conditions affecting cognitive abilities (e.g., memory) as well as conditions generally related to all types of dementia. “Neurodegenerative diseases” may be associated with impairment or loss of cognitive abilities, potential loss of cognitive abilities and/or impairment or loss of brain cells. Exemplary “neurodegenerative diseases” include Alzheimer's disease, Parkinson's disease, amyotrophic lateral sclerosis (ALS), Down syndrome, dementia, multi-infarct dementia, mild cognitive impairment (MCI), epilepsy, seizures, Huntington's disease, neurodegeneration induced by viral infection (e.g. AIDS, encephalopathies), traumatic brain injuries, as well as ischemia and stroke.

“Neurodegenerative diseases” also includes any undesirable condition associated with the disease. For instance, a method of treating a neurodegenerative disease includes methods of treating or preventing loss of neuronal function characteristic of neurodegenerative disease.

In some embodiments, the compounds of the invention are useful in preventing or reducing the risk of developing any of the diseases referred to herein; e.g., preventing or reducing the risk of developing a disease, condition or disorder in an individual who may be predisposed to the disease, condition or disorder but does not yet experience or display the pathology or symptomatology of the disease.

### Combination Therapies

One or more additional pharmaceutical agents or treatment methods can be used in combination with a compound of Formula I for treatment of LRRK2-associated diseases, disorders, or conditions, or diseases or conditions as described herein. The agents can be combined with the present compounds in a single dosage form, or the agents can be administered simultaneously or sequentially as separate dosage forms. In some embodiments, the additional pharmaceutical agent is a dopamine precursor, including, for example, levodopa, melevodopa, and etilevodopa. In some embodiments, the additional pharmaceutical agent is a dopamine agonist, including, for example, pramipexole, ropinorole, apomorphine, rotigotine, bromocriptine, cabergoline, and pergolide. In some embodiments, the additional pharmaceutical agent is a monamine oxidase B (“MAO B”) inhibitor, including, for example, selegiline and rasagiline. In some embodiments, the additional pharmaceutical agent is a catechol O-methyltransferase (“COMT”) inhibitor, including, for example, tolcapone and entacapone. In some embodiments, the additional pharmaceutical agent is an anticholinergic agent including, for example, benztropine, trihexyphenidyl, procyclidine, and biperiden. In some embodiments, the additional pharmaceutical agent is a glutamate (“NMDA”) blocking drug, including, for example, amantadine. In some embodiments, the additional pharmaceutical agent is an adenosine A2A antagonist, including, for example, istradefylline and preladenant. In some embodiments, the additional pharmaceutical agent is a 5-HTTa antagonist, including, for example, piclozotan and pardoprunox. In some embodiments, the additional pharmaceutical agent is an alpha 2 antagonist, including, for example, atipamezole and fipamezole.

### Formulations, Dosage Forms, and Administration

When employed as pharmaceuticals, the compounds of the present disclosure can be administered in the form of pharmaceutical compositions. Thus the present disclosure provides a composition comprising a compound of Formula I or any of the formulas as described herein, a compound as recited in any of the claims and described herein, or a pharmaceutically acceptable salt thereof, or any of the embodiments thereof, and at least one pharmaceutically acceptable carrier. These compositions can be prepared in a manner well known in the pharmaceutical arts, and can be administered by a variety of routes, depending upon whether local or systemic treatment is indicated and upon the area to be treated. Administration may be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral or parenteral. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or may be, e.g., by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

This invention also includes pharmaceutical compositions which contain, as the active ingredient, the compound of the present disclosure or a pharmaceutically acceptable salt thereof, in combination with one or more pharmaceutically acceptable carriers. In some embodiments, the composition is suitable for topical administration. In making the compositions of the invention, the active ingredient is typically mixed with an excipient, diluted by an excipient or enclosed within such a carrier in the form of, e.g., a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, e.g., up to 10% by weight of the active compound, soft and hard gelatin capsules, suppositories, sterile injectable solutions and sterile packaged powders.

In some embodiments, the composition is a sustained release composition comprising at least one compound described herein, or a pharmaceutically acceptable salt thereof, and at least one pharmaceutically acceptable carrier or excipient

The compositions can be formulated in a unit dosage form, each dosage containing from about 5 to about 1,000 mg (1 g). The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

The active compound may be effective over a wide dosage range and is generally administered in a therapeutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms and the like.

The therapeutic dosage of a compound of the present invention can vary according to, e.g., the particular use for which the treatment is made, the manner of administration of the compound, the health and condition of the patient, and the judgment of the prescribing physician. The proportion or concentration of a compound of the invention in a pharmaceutical composition can vary depending upon a number of factors including dosage, chemical characteristics (e.g., hydrophobicity), and the route of administration. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

The liquid forms in which the compounds and compositions of the present invention can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions can be nebulized by use of inert gases. Nebulized solutions may be breathed directly from the nebulizing device or the nebulizing device can be attached to a face mask, tent, or intermittent positive pressure breathing machine. Solution, suspension, or powder compositions can be administered orally or nasally from devices which deliver the formulation in an appropriate manner.

Topical formulations can contain one or more conventional carriers. In some embodiments, ointments can contain water and one or more hydrophobic carriers.

### Examples

Experimental procedures for compounds of the invention are provided below. Where the preparation of starting materials is not described, these are commercially available, known in the literature, or readily obtainable by those skilled in the art using standard procedures. Where it is stated that compounds were prepared analogously to earlier examples or intermediates, it will be appreciated by the skilled person that the reaction time, number of equivalents of reagents and temperature can be modified for each specific reaction and that it may be necessary or desirable to employ different work-up or purification techniques. Where reactions are carried out using microwave irradiation, the microwave used is a Biotage Initiator. The actual power supplied varies during the course of the reaction in order to maintain a constant temperature.

All solvents used were commercially available and were used without further purification. Reactions were typically run using anhydrous solvents under an inert atmosphere of nitrogen.

Liquid chromotography conditions are described below.

Liquid Chromatography—Mass spectrometry Method A

HPLC Conditions: Chiralpak AS-H (25*2.0 cm, 5 μm); Mobile phase: n-hexane/(EtOH+0.1% isopropylamine) 65/35% v/v; Flow rate: 17 mL/min; DAD detection 220 nm Loop 750 μL; Total amount: 32 mg. Solubilization: 32 mg in 3.0 mL (EtOH/MeOH 1/1)=10.6 mg/mL. Injection: 8 mg/injection.

Liquid Chromatography—Mass spectrometry Method B

HPLC Conditions: Column Chiralpak IC (25*2.0 cm, 5 μm); Mobile phase: n-hexane/(EtOH+0.1% isopropylamine) 60/40% v/v; Flow rate: 17 mL/min; DAD detection 220 nm Loop 800 μL: Total amount: 23 mg. Solubilization 23 mg in 2.5 mL (EtOH/MeOH 1/1)=9.2 mg/mL. Injection: 7.4 mg/injection.

Liquid Chromatography—Mass spectrometry Method C

SFC conditions: Chiralpak AS-H (25*2.0 cm, 5 μm); Mobile phase: n-hexane (EtOH+0.1% isopropaylamine) 15%; Flow rate: 45 mL/min.

Liquid Chromatography—Mass spectrometry Method D

HPLC Conditions: Chiralpak AD-H (25*2.0 cm, 5 μm); Mobile phase: n-hexane/(EtOH+0.1% isopropylamine) 60/40% v/v; Flow rate: 17 mL/min; DAD detection 220 nm Loop 800 μL; Total amount 52 mg. Solubilization 52 mg in 5.0 mL 1,1,1,3,3,3-hexafluoro-2-propanol (EtOH/MeOH) 1/1=10.4 mg/mL. Injection: 8.32 mg/injection.

Liquid Chromatography—Mass spectrometry Method E

HPLC Conditions: Chiralpak AD-H (25*2.0 cm, 5 μm; Mobile phase: n-hexane/(EtOH+0.1% isopropylamine) 60/40% v/v; Flow rate: 17 mL/min; DAD detection 220 nm Loop 1250 μL; Total amount 32 mg. Solubilization 32 mg in 6.0 mL 1,1,1,3,3,3-hexafluoro-2-propanol/(EtOH/MeOH) 1/1=5.3 mg/mL. Injection: 6.6 mg/injection.

### Liquid Chromatography-Mass Spectrometry Method F

HPLC Conditions: MDAP Method; LC/MS System: Fractionlynx (Waters) with QDa MS detector; Column: XSelect CSH Prep. C18 OBD (30*100 mm, 5 μm); Mobile phase: A=H₂O+0.1% HCOOH; B=MeCN 60/40% v/v; Flow rate: 40 mL/min; UV detection range: 210 nm to 350 nm; Loop: 1 mL.

### Liquid Chromatography-Mass Spectrometry Method G

HPLC Conditions: MDAP Method; LC/MS System: Fractionlynx (Waters) with QDa MS detector; Column: XSelect CSH Prep. C18 OBD (30*100 mm, 5 μm); Mobile phase: A=H₂O+0.1% HCOOH; B=MeCN 65/35% v/v; Flow rate: 40 mL/min; UV detection range: 210 nm to 350 nm; Loop: 1 mL.

### Liquid Chromatography-Mass Spectrometry Method H

HPLC Conditions: Chiralpak IC (25*2.0 cm, 5 μm); Mobile phase: n-hexane/(EtOH+0.1% isopropylamine) 55/45% v/v; Flow rate: 17 mL/min; DAD detection 220 nm Loop 1000 μL; Total amount 3.0 mg. Solubilization: 3.0 mg in 2.0 mL (EtOH/MeOH 1/1)=1.5 mg/mL. Injection 1.5 mg/injection.

### Liquid Chromatography-Mass Spectrometry Method I

HPLC Conditions: Chiralpak AD-H (25*2.0 cm, 5 μm); Mobile phase: n-hexane/(EtOH+0.1% isopropylamine) 60/40% v/v; Flow rate: 17 mL/min; DAD detection 220 nm Loop 850 μL; Total amount 25 mg. Solubilization: 25 mg in 3.0 mL (EtOH/MeOH 1/1)=8.3 mg/mL. Injection 8.3 mg/injection.

### Liquid Chromatography-Mass Spectrometry Method J

HPLC Conditions: MDAP Method; LC/MS System: Fractionlynx (Waters) with QDa MS detector; Column: XSelect CSH Prep. C18 OBD (30*100 mm, 5 μm); Mobile phase: A=H₂O+0.1% HCOOH; B=MeCN 67/33% v/v; Flow rate: 40 mL/min; UV detection range: 210 nm to 350 nm; Loop: 1 mL.

### Liquid Chromatography-Mass Spectrometry Method: K

HPLC Conditions: Chiralpak AS-H (25*2.0 cm, 5 μm); Mobile phase: n-hexane/(EtOH+0.1% isopropylamine) 60/40% v/v; Flow rate: 17 mL/min; DAD detection 220 nm Loop 1000 μL; Total amount 28 mg. Solubilization 28 mg in 4.5 mL 1,1,1,3,3,3-hexafluoro-2-propanol/(EtOH/MeOH) 1/1=6.2 mg/mL. Injection 6.2 mg/injection.

### Liquid Chromatography-Mass Spectrometry Method: L

HPLC Conditions: MDAP Method; LC/MS System: Fractionlynx (Waters) with QDa MS detector; Column: XSelect CSH Prep. C18 OBD (30*100 mm, 5 μm); Mobile phase: A=H₂O+0.1% HCOOH; B=MeCN 60/40% v/v; Flow rate: 40 mL/min; UV detection range: 210 nm to 350 nm; Loop: 1 mL.

### Liquid Chromatography-Mass Spectrometry Method: M

SFC Conditions: Chiralcel OJ-H (25*2.0 cm, 5 μm); Mobile phase: (MeOH+0.1% isopropylamine) 45%; Flow rate: 45 mL/min; UV detection 220 nm; Loop 700 μL; Total amount 280 mg. Sample preparation 280 mg in 10 mL EtOH/MeOH 1/1=30 mg/mL. Injection 21 mg.

### Liquid Chromatography-Mass Spectrometry Method: N

HPLC Conditions: MDAP Method; LC/MS System: Fractionlynx (Waters) with QDa MS detector; Column: XSelect CSH Prep. C18 OBD (30*100 mm, 5 μm); Mobile phase: A=H₂O+0.1% HCOOH; B=MeCN 70/30% v/v; Flow rate: 40 mL/min; UV detection range: 210 nm to 350 nm; Loop: 1 mL.

### Liquid Chromatography-Mass Spectrometry Method: O

SFC Conditions: Chiralpak AS-H (25*2.0 cm, 5 μm); Mobile phase: (EtOH+0.1% isopropylamine) 20%; Flow rate: 45 mL/min; UV detection 220 nm Loop 200 μL; Total amount 175 mg. Sample preparation: 175 mg in 3.0 mL 1,1,1,3,3,3-hexafluoro-2-propanol=58.3 mg/mL. Injection 11.7 mg.

Liquid Chromatography—Mass spectrometry Method P

HPLC Conditions: Chiralpak AS-H (25*0.46 cm, 5 μm); Mobile phase: n-hexane/(EtOH+0.1% isopropylamine) 60/40% v/v; Flow rate: 1.0 mL/min; DAD 220 nm Loop 20 μL.

Liquid Chromatography—Mass spectrometry Method Q

HPLC Conditions: Chiralcel OD-H (25*2.0 cm), 5 μm); Mobile phase: n-hexane/(EtOH+0.1% isopropylamine) 80/20% v/v; Flow rate: 17 mL/min; DAD detection 220 nm Loop 100 μL; Total amount 13.2 mg; Solubilization: 13.2 mg in 2.8 mL 1,1,1,3,3,3-hexafluoro-2-propanol/(EtOH/MeOH) 1/1=4.7 mg/mL. Injection: 4.7 mg/injection.

Liquid Chromatography—Mass spectrometry Method R

HPLC Conditions: Chiralpak IC (25*2.0 cm), 5 μm); Mobile phase: n-hexane/(EtOH+0.1% isopropylamine) 70/30% v/v; Flow rate: 17 mL/min; DAD detection 220 nm Loop 850 μL; Total amount 20 mg. Solubilization: 20 mg in 2.5 mL DCM/(EtOH/MeOH) 1/1=8 mg/mL. Injection 6.8 mg/injection.

Liquid Chromatography—Mass spectrometry Method S

HPLC Conditions: Chiralpak AD-H (25*2.0 cm, 5 μm); Mobile phase: n-hexane/(EtOH+0.1% isopropylamine) 65/35% v/v; Flow rate: 17 mL/min; DAD detection 220 nm Loop 800 μL; Total amount 50 mg. Solubilization: 50 mg in 5.0 mL 1,1,1,3,3,3-hexafluoro-2-propanol/(EtOH/MeOH 1/1)=10 mg/mL. Injection: 8.0 mg/injection.

Liquid Chromatography—Mass spectrometry Method T

HPLC Conditions: Chiralpak AD-H (25*2.0 cm, 5 μm); Mobile phase: n-hexane/(EtOH+0.1% isopropylamine) 55/45% v/v; Flow rate: 17 mL/min; DAD detection 220 nm Loop 800 μL; Total amount 60 mg. Solubilization: 60 mg in 5.0 mL (EtOH/MeOH 1/1)=12 mg/mL. Injection: 9.6 mg/injection.

Liquid Chromatography—Mass spectrometry Method U

HPLC Conditions: Chiralpak IC (25*2.0 cm), 5 μm); Mobile phase: n-hexane/(EtOH+0.1% isopropylamine) 60/40% v/v; Flow rate: 17 mL/min; DAD detection 220 nm Loop 800 μL; Total amount 49 mg. Solubilization: 49 mg in 2.0 mL DCM/(EtOH/MeOH 1/1)=10.9 mg/mL. Injection 8.7 mg/injection.

Liquid Chromatography—Mass spectrometry Method V

SFC Conditions: Chiralpak AS, (250*30, 10 um); Mobile phase: A for CO₂ and B for MeOH(0.1% NH₃.H₂O); gradient: B %=33% isocratic elution mode; Flow rate: 60 g/min.

Liquid Chromatography—Mass spectrometry Method W

HPLC Conditions: Waters Xbridge BEH C 18 (100*30 mm, 10 um); Mobile phase: [water(10 mM NH₄HCO₂)-ACN]; B %: 23%-53%, 8 min.

Liquid Chromatography—Mass spectrometry Method X

SFC Conditions: DAICEL CHIRALPAK AS (250 mm*30 mm, 10 um); Mobile phase: [0.1% NH₃.H₂O EtOH]; B %: 50%-50%, 12 min.

Liquid Chromatography—Mass spectrometry Method Y

HPLC Conditions: Waters Xbridge BEH C18 (100*30 mm, 10 um); Mobile phase: [water (10 mM NH₄HCO₂)-ACN]; B %: 28%-58%, 8 min.

Liquid Chromatography—Mass spectrometry Method Z

SFC Conditions: Chiralpak AD (250*30, 10 um); Mobile phase: A for CO₂ and B for MeOH (0.1% NH₃.H₂O); gradient: B %=42% isocratic elution mode; Flow rate: 70 g/min.

Liquid Chromatography—Mass spectrometry Method AA

SFC Conditions: Chiralpak IG (250*30 mm, 10 um); Mobile phase: A for CO₂ and B for MeOH(0.1% NH₃.H₂O); gradient: B %=50% isocratic mode; Flow rate: 80 g/min; wavelength: 220 nm.

Liquid Chromatography—Mass spectrometry Method AB

SFC Conditions: Chiralpak OJ (250*30 mm, 10 um); Mobile phase: A for CO₂ and B for EtOH(0.1% NH₃.H₂O); gradient: B %=41% isocratic elution mode; Flow rate: 68 g/min; wavelength: 220 nm.

Liquid Chromatography—Mass spectrometry Method AC

HPLC conditions: Waters Xbridge BEH C18 (100*30 mm, 10 um); Mobile phase: [water (10 mM NH₄HCO₃)-ACN]; B %: 35%-65%.

Liquid Chromatography—Mass spectrometry Method AD

SFC conditions: CHIRALPAK AY (250*30 mm, 10 um); Mobile phase: A for CO₂ and B for EtOH (0.1% NH₄OH); Gradient: B %=46% isocratic elution mode; Flow rate: 75 g/min; Wavelength: 220 nm.

Liquid Chromatography—Mass spectrometry Method AE

HPLC conditions: Phenomenex Gemini-NX C18 (75*30 mm, 3 um); Mobile phase: [water (10 mM NH₄HCO₃)-ACN]; B %: 30%-50%.

Liquid Chromatography—Mass spectrometry Method AF

HPLC conditions: DAICEL CHIRALCEL OJ (250*30 mm, 10 um); Mobile phase: [water (0.1% NH₄OH)-IPA]; B %: 35%.

Liquid Chromatography—Mass spectrometry Method AG

HPLC conditions: Phenomenex Gemini-NX C18 (75*30 mm, 3 um); Mobile phase: [water (10 mM NH₄HCO₃)-ACN]; B %: 33%-53%.

Liquid Chromatography—Mass spectrometry Method AH

HPLC conditions: DAICEL CHIRALPAK AD (250*30 mm, 10 um); Mobile phase: [water (0.1% NH₄OH)-IPA]; B %: 35%.

Liquid Chromatography—Mass spectrometry Method AI

HPLC conditions: Phenomenex Synergi C18 (150*25 mm, 10 um); Mobile phase: [water (0.1% TFA)-ACN]; B %: 35%-55%.

Liquid Chromatography—Mass spectrometry Method AJ

HPLC conditions: DAICEL CHIRALPAK AS (250*30 mm, 10 um); Mobile phase: [water (0.1% NH₄OH)-EtOH]; B %: 50%.

Liquid Chromatography—Mass spectrometry Method AK

HPLC conditions: Phenomenex Luna C18 (200*40 mm, 10 um); Mobile phase: [water (0.1% TFA)-ACN]; B %: 15%-47%.

Liquid Chromatography—Mass spectrometry Method AL

SFC conditions: DAICEL CHIRALCEL OJ (250*30 mm, 10 um); Mobile phase: [water (0.1% NH₄OH)-MeOH]; B %: 45%.

Liquid Chromatography—Mass spectrometry Method AM

HPLC conditions: Phenomenex Luna C18 (200*40 mm, 10 um); Mobile phase: [water (0.1% TFA)-ACN]; B %: 15%-45%.

Liquid Chromatography—Mass spectrometry Method AN

SFC conditions: REGIS (s,s) WHELK-01 (250*50 mm, 10 um); Mobile phase: [water (0.1% NH₄OH)-EtOH]; B %: 50%.

Liquid Chromatography—Mass spectrometry Method AO

HPLC conditions: Phenomenex Gemini-NX C18 (75*30 mm, 3 um); Mobile phase: [water (10 mM NH₄HCO₃)-ACN]; B %: 30%-50%.

Liquid Chromatography—Mass spectrometry Method AP

HPLC conditions: Phenomenex Gemini-NX C18 (75*30 mm, 3 um); Mobile phase: [water (10 mM, NH₄HCO₃)-ACN]; B %: 15%-35%.

Liquid Chromatography—Mass spectrometry Method AQ

HPLC conditions: DAICEL CHIRALPAK IC (250*30 mm, 5 um); Mobile phase: [water (0.1% NH₄OH), EtOH]; B %: 40%.

Liquid Chromatography—Mass spectrometry Method AR

HPLC conditions: Phenomenex Synergi C18 (150*25 mm, 10 um); Mobile phase: [water (0.1% TFA)-ACN]; B %: 10%-30%.

Liquid Chromatography—Mass spectrometry Method AS

HPLC conditions: REGIS (s, s) WHELK-01 (250*50 mm, 10 um); Mobile phase: [water (0.1% NH₄OH)-MeOH]; B %: 60%.

Liquid Chromatography—Mass spectrometry Method AT

HPLC conditions: Phenomenex luna C18 (100*40 mm, 5 um); Mobile phase: [water (0.1% TFA)-ACN]; B %: 1%-33%.

Liquid Chromatography—Mass spectrometry Method AU

HPLC conditions: Phenomenex-Cellulose-2 (250*30 mm, 10 um); Mobile phase: [water (0.1% NH₄OH)-EtOH]; B %: 55%.

Liquid Chromatography—Mass spectrometry Method AV

HPLC conditions: Phenomenex Luna (80*30 mm, 3 um); Mobile phase: [water (0.1% TFA)-ACN]; B %: 20%-50%.

Liquid Chromatography—Mass spectrometry Method AW

HPLC conditions: DAICEL CHIRALPAK AD (250*30 mm, 10 um); Mobile phase: [0.1% NH₄OH, EtOH]; B %: 50%.

Liquid Chromatography—Mass spectrometry Method AX

HPLC conditions: Regis (s,s) WHELK-01 (250*30 mm, 5 um); Mobile phase: [0.1% NH₄OH, IPA]; B %: 45%.

Liquid Chromatography—Mass spectrometry Method AY

HPLC conditions: Phenomenex Gemini-NX (150*30 mm, 5 um); Mobile phase: [water (10 mm, NH₄HCO₃)-ACN]; B %: 40%-60%.

Liquid Chromatography—Mass spectrometry Method AZ

HPLC conditions: DAICEL CHIRALPAK AS(250*30 mm, 10 um); Mobile phase: [MeOH]; B %: 50%.

Liquid Chromatography—Mass spectrometry Method BA

HPLC conditions: Phenomenex Luna C18 (150*30 mm, 5 um); Mobile phase: [water (0.1% TFA)-ACN]; B %: 20%-50%.

Liquid Chromatography—Mass spectrometry Method BB

HPLC conditions: Phenomenex Luna (80*30 mm, 3 um); Mobile phase: [water (0.1% TFA)-ACN]; B %: 35%-65%.

Liquid Chromatography—Mass spectrometry Method BC

HPLC conditions: DAICEL CHIRALPAK AD (250*30 mm, 10 um); Mobile phase: [0.1% NH₄OH, EtOH]; B %: 50%.

Liquid Chromatography—Mass spectrometry Method BD

HPLC conditions: Phenomenex Gemini-NX C18 (75*30 mm, 3 um); Mobile phase: [water (0.1% TFA)-ACN]; B %: 20%-50%.

Liquid Chromatography—Mass spectrometry Method BE

HPLC conditions: DAICEL CHIRALCEL OJ (250*30 mm, 10 um); Mobile phase: [water (0.1% NH₄OH)-MeOH]; B %: 50%.

Liquid Chromatography—Mass spectrometry Method BF

HPLC conditions: DAICEL CHIRALPAK AD (250*30 mm, 10 um); Mobile phase: [water (0.1% NH₄OH)-EtOH]; B %: 50%.

Liquid Chromatography—Mass spectrometry Method BG

HPLC conditions: Phenomenex Synergi C18 (150*25 mm, 10 um); Mobile phase: [water (0.1% TFA)-ACN]; B %: 20%-40%.

Liquid Chromatography—Mass spectrometry Method BH

HPLC conditions: DAICEL CHIRALPAK AS (250*30 mm, 10 um); Mobile phase: [water (0.1% NH₄OH)-IPA]; B %: 50%.

Liquid Chromatography—Mass spectrometry Method BI

HPLC conditions: Phenomenex Gemini-NX C18 (75*30 mm, 3 um); Mobile phase: [water (0.05% NH₄OH+10 mM NH₄HCO₃)-ACN]; B %: 30%-60%.

Liquid Chromatography—Mass spectrometry Method BJ

HPLC conditions: DAICEL CHIRALPAK IG (250*30 mm, 10 um); Mobile phase: [water (0.1% NH₄OH)-EtOH]; B %: 60%.

Liquid Chromatography—Mass spectrometry Method BK

HPLC conditions: Phenomenex Gemini-NX C18 (75*30 mm, 3 um); Mobile phase: [water (10 mM NH₄HCO₃)-ACN]; B %: 18%-58%.

Liquid Chromatography—Mass spectrometry Method BL

HPLC conditions: DAICEL CHIRALCEL OJ (250*30 mm, 10 um); Mobile phase: [water (0.1% NH₄OH)-MeOH]); B %: 45%.

Liquid Chromatography—Mass spectrometry Method BM

HPLC conditions: DAICEL CHIRALPAK AS (250*30 mm, 10 um); Mobile phase: [water (0.1% NH₄OH)-EtOH]; B %: 50%.

Liquid Chromatography—Mass spectrometry Method BN

HPLC conditions: Phenomenex Luna C18 (100*30 mm, 5 um); Mobile phase: [water (0.1% TFA)-ACN]; B %: 35%-47%.

Liquid Chromatography—Mass spectrometry Method BO

HPLC conditions: Phenomenex Luna C18 (100*40 mm, 5 um); Mobile phase: [water (0.1% TFA)-ACN]; B %: 20%-48%.

Liquid Chromatography—Mass spectrometry Method BP

HPLC conditions: Phenomenex Gemini-NX (80*40 mm, 3 um); Mobile phase: [water (10 mM NH₄HCO₃)-ACN]; B %: 30%-50%.

Liquid Chromatography—Mass spectrometry Method BQ

HPLC conditions: Phenomenex Gemini-NX C18 (75*30 mm, 3 um); Mobile phase: [water (10 mM NH₄HCO₃)-ACN]; B %: 25%-55%.

Liquid Chromatography—Mass spectrometry Method BR

HPLC conditions: Phenomenex Luna C18 (150*30 mm, 5 um); Mobile phase: [water (0.1% TFA)-ACN]; B %: 10%-45%.

Liquid Chromatography—Mass spectrometry Method BS

HPLC conditions: Phenomenex Luna C18 (150*30 mm, 5 um); Mobile phase: [water (0.1% TFA)-ACN]; B %: 35%-55%.

Liquid Chromatography—Mass spectrometry Method BT

HPLC conditions: DAICEL CHIRALPAK AD (250*30 mm, 10 um); Mobile phase: [0.1% NH₄OH, EtOH]; B %: 50%.

Liquid Chromatography—Mass spectrometry Method BU

HPLC conditions: Phenomenex Luna C18 (100*30 mm, 5 um); Mobile phase: [water (0.1% TFA)-ACN]; B %: 15%-45%.

Liquid Chromatography—Mass spectrometry Method BV

HPLC conditions: DAICEL CHIRALPAK AD (250*30 mm, 10 um); Mobile phase: [water (0.1% NH₄OH)-EtOH]; B %: 45%.

Liquid Chromatography—Mass spectrometry Method BW

HPLC conditions: Phenomenex Luna C18 (100*40 mm, 5 um); Mobile phase: [water (0.1% TFA)-ACN]; B %: 10%-40%.

Liquid Chromatography—Mass spectrometry Method BX

HPLC conditions: DAICEL CHIRALPAK AD (250*30 mm, 10 um); Mobile phase: [water (0.1% NH₄OH)-EtOH]; B %: 60%.

Liquid Chromatography—Mass spectrometry Method BY

HPLC conditions: Phenomenex Luna C18 (100*40 mm, 5 um); Mobile phase: [water (0.1% TFA)-ACN]; B %: 10%-40%.

Liquid Chromatography—Mass spectrometry Method BZ

HPLC conditions: REGIS (s,s) WHELK-01 (250*30 mm, 5 um); Mobile phase: [water (0.1% NH₄OH)-EtOH]; B %: 45%.

Liquid Chromatography—Mass spectrometry Method CA

HPLC conditions: DAICEL CHIRALPAK AD (250*30 mm, 10 um); Mobile phase: [water (0.1% NH₄OH)-IPA]; B %: 40%.

Liquid Chromatography—Mass spectrometry Method CB

HPLC conditions: Phenomenex Gemini-NX C18 (75*30 mm, 3 um); Mobile phase: [water (10 mM NH₄HCO₃)-ACN]; B %: 35%-60%.

Liquid Chromatography—Mass spectrometry Method CC

HPLC conditions: DAICEL CHIRALPAK AD (250*30 mm, 10 um); Mobile phase: [water (0.1% NH₄OH)-EtOH]; B %: 42%.

Liquid Chromatography—Mass spectrometry Method CD

HPLC conditions: Phenomenex Luna C18 (100*40 mm, 5 um); Mobile phase: [water (0.1% TFA)-ACN]; B %: 20%-50%.

Liquid Chromatography—Mass spectrometry Method CE

HPLC conditions: DAICEL CHIRALPAK AS (250*30 mm, 10 um); Mobile phase: [water (0.1% NH₄OH)-EtOH]; B %: 45%.

Liquid Chromatography—Mass spectrometry Method CF

HPLC conditions: Waters Xbridge BEH C18 (100*25 mm, 5 um); Mobile phase: [water (10 mM NH₄HCO₃)-ACN]; B %: 40%-65%.

Liquid Chromatography—Mass spectrometry Method CG

HPLC conditions: DAICEL CHIRALPAK AS (250*30 mm, 10 um); Mobile phase: [water (0.1% NH₄OH)-EtOH]; B %: 40%.

Liquid Chromatography—Mass spectrometry Method CH

HPLC conditions: DAICEL CHIRALPAK AS (250*30 mm, 10 um); Mobile phase: [water (0.1% NH₄OH)-EtOH]; B %: 47%.

Liquid Chromatography—Mass spectrometry Method CI

HPLC conditions: Phenomenex Luna C18 (100*40 mm, 5 um); Mobile phase: [water (0.1% TFA)-ACN]; B %: 35%-70%.

Liquid Chromatography—Mass spectrometry Method CJ

HPLC conditions: Phenomenex Gemini-NX C18 (75*30 mm, 3 um); Mobile phase: [water (10 mM NH₄HCO₃)-ACN]; B %: 45%-75%.

Liquid Chromatography—Mass spectrometry Method CK

HPLC conditions: Waters Xbridge BEH C18 (100*30 mm, 10 um); Mobile phase: [water (10 mM NH₄HCO₃)-ACN]; B %: 15%-45%.

Liquid Chromatography—Mass spectrometry Method CL

HPLC conditions: Phenomenex Luna C18 (150*30 mm, 5 um); Mobile phase: [water (0.1% TFA)-ACN]; B %: 20%-50%.

Liquid Chromatography—Mass spectrometry Method CM

HPLC conditions: CHIRALPAK AS-H (25*2.0 cm, 5 um); Mobile phase: n-hexane/(EtOH+0.10% isopropylamine) 60/40% v/v, Flow rate: 17 mL/min; DAD detection 220 nm Loop 1000 μL. Total amount; 13 mg. Solubilization 13 mg in 3.0 mL EtOH/MeOH 1/1=4.3 mg/mL, Injection 4.3 mg/injection.

Liquid Chromatography—Mass spectrometry Method CO

HPLC conditions: CHIRALPAK AS-H (25*2.0 cm, 5 um); Mobile phase: n-hexane/(EtOH+0.1% isopropylamine) 65/35% v/v; Flow rate: 17 mL/min; DAD detection 220 nm Loop 750 μL; Total amount: 7 mg. Solubilization: 7 mg in 1.5 mL EtOH/MeOH 1/1=3.5 mg/mL. Injection: 3.5 mg/injection.

Liquid Chromatography—Mass spectrometry Method CQ

HPLC conditions: CHIRALCEL OJ-H (25×2.0 cm, 5 um); Mobile phase: n-hexane/(EtOH+0.1% isopropylamine) 60/40% v/v; Flow rate: 17 mL/min; DAD detection 220 nm Loop 750 μL; Total amount: 50 mg. Solubilization: 50 mg in 3.5 mL EtOH/MeOH 1/1=14.2 mg/mL. Injection: 10.7 mg/injection.

Liquid Chromatography—Mass spectrometry Method CR

HPLC conditions: CHIRALPAK AD-H (25*2.0 cm, 5 um); Mobile phase: n-hexane/(EtOH/MeOH 1/1+0.1% isopropylamine) 50/50% v/v; Flow rate: 17 mL/min; DAD detection 220 Loop 300 μL; Total amount: 90 mg. Solubilization: 90 mg in 2.7 mL 1,1,1,3,3,3-hexafluoro-2-propanol=33.3 mg/mL. Injection: 10 mg/injection.

Liquid Chromatography—Mass spectrometry Method CS

HPLC conditions: Chiralcel OJ-H (25*0.0 cm, 5 um); Mobile phase: n-hexane/(EtOH/MeOH 1/1+0.1% isopropylamine) 50/50% v/v; Flow rate: 17 mL/min; DAD detection 220 nm Loop 1000 μL; Total amount: 12 mg. Solubilization 12 mg in 2.0 mL EtOH/MeOH 1/1=6.0 mg/mL. Injection: 6.0 mg/injection.

Liquid Chromatography—Mass spectrometry Method CT

HPLC conditions: Chiralpak AS-H (25*2.0 cm, 5 um); Mobile phase: n-hexane/(EtOH/MeOH+0.1% isopropylamine) 65/35% v/v; Flow rate: 17 mL/min; DAD detection 220 Loop 1000 μL; Total amount: 45 mg. Solubilization 55 mg in 7.0 mL 1,1,1,3,3,3-hexafluoro-2-propanol/(EtOH/MeOH 1/1) 1/1=6.4 mg/mL. Injection: 6.4 mg/injection.

Liquid Chromatography—Mass spectrometry Method CU

HPLC conditions: Chiralcel OJ-H (25*2.0 cm, 5 um); Mobile phase: n-hexane/(EtOH+0.1% isopropylamine) 60/40% v/v; Flow rate: 17 mL/min; DAD detection 220 nm Loop 800 μL; Total amount: 50 mg. Solubilization 50 mg in 6.0 mL 1,1,1,3,3,3-hexafluoro-2-propanol/(EtOH/MeOH 1/1) 1/1=8.3 mg/mL. Injection: 6.6 mg/injection.

Liquid Chromatography—Mass spectrometry Method CV HPLC conditions and results: Chiralpak AS-H (25*2.0 cm, 5 um); Mobile phase: n-hexane/(EtOH+0.1% isopropylamine) 70/30% v/v; Flow rate: 17 mL/min; DAD detection 220 Loop 700 μL; Total amount: 65 mg. Solubilization 65 mg in 4.0 mL EtOH/MeOH 1/1=16.25 mg/mL. Injection: 11.4 mg/injection.
Liquid Chromatography—Mass spectrometry Method CW

HPLC conditions: Chiralpak AD-H (25*2.0 cm, 5 um); Mobile phase: n-hexane/(EtOH+0.1% isopropylamine) 60/40% v/v; Flow rate; 17 mL/min; DAD detection 220 Loop 300 μL. Total amount; 121 mg. Solubilization 121 mg in 2.5 mL 1,1,1,3,3,3-hexafluoro-2-propanol=48.4 mg/mL. Injection: 14.5 mg/injection.

Liquid Chromatography—Mass spectrometry Method CX

HPLC conditions: Chiralpak IC (25*2.0 cm, 5 um); Mobile phase: n-hexane/(EtOH+0.1% isopropylamine) 65/35% v/v; Flow rate; 17 mL/min; DAD detection 220 nm Loop 1000 μL; Total amount: 13.5 mg. Solubilization 13.5 mg in 2.0 mL (EtOH/MeOH) 1/1=6.7 mg/mL. Injection: 6.7 mg/injection.

Liquid Chromatography—Mass spectrometry Method CY

HPLC conditions: Chiralpak AD-H (25*2.0 cm, 5 um); Mobile phase: n-hexane/(EtOH/MeOH 1/1+0.1% isopropylamine) 45/55% v/v; Flow rate; 17 mL/min; DAD detection 220 nm Loop 1000 μL. Total amount: 20 mg. Solubilization 20 mg in 2.0 mL 1,1,1,3,3,3-hexafluoro-2-propanol/(EtOH/MeOH 1/1) 1/1=10.0 mg/mL. Injection: 10.0 mg/injection.

Liquid Chromatography—Mass spectrometry Method DA

HPLC conditions: Chiralpak IC (25×2.0 cm, 5 um); Mobile phase: n-hexane/(EtOH+0.1% isopropylamine) 80/20% v/v; Flow rate; 17 mL/min; DAD detection 220 nm Loop 1000 μL. Total amount: 21 mg. Solubilization 21 mg in 1.5 mL EtOH/MeOH 1/1=14 mg/mL. Injection: 7 mg/injection.

Liquid Chromatography—Mass spectrometry Method DB

HPLC conditions: Chiralpak IC (25*2.0 cm, 5 um); mobile phase: n-hexane/(EtOH+0.1% isopropylamine) 60/40% v/v; Flow rate; 17 mL/min; DAD detection 220 nm Loop 1000 μL; Total amount: 12.5 mg. Solubilization: 12.5 mg in 2.0 mL (EtOH/MeOH) 1/1=6.2 mg/mL. Injection 6.2 mg/injection.

Liquid Chromatography—Mass spectrometry Method DC

HPLC conditions: Chiralpak AS-H (25*2.0 cm, 5 um); mobile phase: n-hexane/(EtOH+0.1% isopropylamine) 65/35% v/v; Flow rate 17 mL/min; DAD detection 220 Loop 1000 μL; Total amount: 60 mg. Solubilization: 60 mg in 6.5 mL 1,1,1,3,3,3-hexafluoro-2-propanol/(EtOH/MeOH 1/1) 1/1=9.2 mg/mL. Injection 9.2 mg/injection.

Liquid Chromatography—Mass spectrometry Method DD

HPLC conditions: Chiralpak AS-H (25*2.0 cm, 5 um); Mobile phase: n-hexane/(EtOH+0.1% isopropylamine) 75/25% v/v; Flow rate; 17 mL/min; DAD detection 220 Loop 1000 μL; Total amount: 30 mg. Solubilization: 30 mg in 3.0 mL/(EtOH/MeOH 1/1) 1/1=10 mg/mL. Injection: 10 mg/injection.

Liquid Chromatography—Mass spectrometry Method DE

HPLC conditions: Chiralpak AS-H (25*2.0 cm, 5 um); Mobile phase: n-hexane/(EtOH+0.1% isopropylamine) 70/30% v/v; Flow rate; 17 mL/min; DAD detection 220 nm Loop 2500 μL; Total amount: 65 mg. Solubilization: 65 mg in 10.0 mL 1,1,1,3,3,3-hexafluoro2-propanol/(EtOH/MeOH 1/1) 1/1=6.5 mg/mL. Injection: 16.2 mg/injection.

Liquid Chromatography—Mass spectrometry Method DF

HPLC conditions: Chiralpak AS-H (25*2.0 cm, 5 um); Mobile phase: n-hexane/(EtOH+0.1% isopropylamine) 60/40% v/v; Flow rate; 17 mL/min; DAD detection 220 nm Loop 2500 μL; Total amount: 50 mg. Solubilization: 50 mg in 9.0 mL 1,1,1,3,3,3-hexafluoro2-propanol/(EtOH/MeOH 1/1) 1/1=5.3 mg/mL. Injection: 14 mg/injection.

Liquid Chromatography—Mass spectrometry Method DG

HPLC conditions: Chiralpak IC (25*2.0 cm, 5 um); Mobile phase: n-hexane/(EtOH+0.1% isopropylamine) 70/30% v/v; Flow rate; 17 mL/min; DAD detection 220 nm Loop 750 μL; Total amount: 40 mg. Solubilization: 40 mg in 5.5 mL DCM/(EtOH/MeOH 1/1) 1/1=7.2 mg/mL. Injection: 5.5 mg/injection.

Liquid Chromatography—Mass spectrometry Method DH

HPLC conditions: Chiralpak IC (25*2.0 cm, 5 um); Mobile phase: n-hexane/(EtOH+0.1% isopropylamine) 60/40% v/v; Flow rate; 17 mL/min; DAD detection 220 nm Loop 1000 μL; Total amount: 80 mg. Solubilization: 80 mg in 8.0 mL EtOH/MeOH 1/1=10 mg/mL. Injection: 10 mg/injection.

Liquid Chromatography—Mass spectrometry Method DI

HPLC conditions: Chiralpak IC (25*2.0 cm, 5 um); Mobile phase: n-hexane/(EtOH+0.1% isopropylamine) 40/60% v/v; Flow rate; 17 mL/min; DAD detection 220 nm Loop 800 μL; Total amount: 48 mg. Solubilization: 48 mg in 4.0 mL DCM/(EtOH/MeOH) 1/1=12 mg/mL. Injection: 9.6 mg/injection.

Liquid Chromatography—Mass spectrometry Method DJ

HPLC conditions: Chiralpak IC (25*2.0 cm, 5 um); Mobile phase: n-hexane/(EtOH+0.1% isopropylamine) 60/40% v/v; Flow rate; 17 mL/min; DAD detection 220 nm Loop 850 μL; Total amount: 30 mg. Solubilization: 30 mg in 2.5 mL EtOH/MeOH 1/1=12 mg/mL. Injection: 10 mg/injection.

Liquid Chromatography—Mass spectrometry Method DK

HPLC conditions: Chiralcel OD-H (25*2.0 cm, 5 um); Mobile phase: n-hexane/(EtOH+0.1% isopropylamine) 60/40% v/v; Flow rate; 17 mL/min; DAD detection 220 nm Loop 1250 μL; Total amount: 60 mg. Solubilization: 60 mg in 8.0 mL 1,1,1,3,3,3-hexafluoro-2-propanol/(EtOH/MeOH 1/1) 1/1=7.5 mg/mL. Injection: 9.4 mg/injection.

Liquid Chromatography—Mass spectrometry Method DL

HPLC conditions: Chiralpak AD-H (25*2.0 cm, 5 um); Mobile phase: n-hexane/(EtOH+0.1% isopropylamine) 60/40% v/v; Flow rate; 17 mL/min; DAD detection 220 nm Loop 500 μL; Total amount: 65 mg. Solubilization: 65 mg in 3.5 mL EtOH/MeOH 1/1=18.6 mg/mL. Injection: 7.1 mg/injection.

Liquid Chromatography—Mass spectrometry Method DM

HPLC conditions: Chiralpak AS-H (25*2.0 cm, 5 um); Mobile phase: n-hexane/(EtOH+0.1% isopropylamine) 70/30% v/v; Flow rate; 17 mL/min; DAD detection 220 nm Loop 400 μL; Total amount: 106 mg. Solubilization: 106 mg in 9.0 mL 1,1,1,3,3,3-hexafluoro-2-propanol/(EtOH/MeOH 1/1) 1/1=11.8 mg/mL. Injection: 4.6 mg/injection.

Liquid Chromatography—Mass spectrometry Method DN

HPLC conditions: Chiralpak AS-H (25*2.0 cm, 5 um); Mobile phase: n-hexane/(EtOH+0.1% isopropylamine) 65/35% v/v; Flow rate; 17 mL/min; DAD detection 220 Loop 1000 μL; Total amount: 57 mg. Solubilization: 57 mg in 6 mL 1,1,1,3,3,3-hexafluoro-2-propanol/(EtOH/MeOH 1/1) 1/1=9.5 mg/mL. Injection: 9.5 mg/injection.

Liquid Chromatography—Mass spectrometry Method DO

HPLC conditions: Chiralpak IC (25*2.0 cm, 5 um); Mobile phase: n-hexane/(EtOH+0.1% isopropylamine) 65/35% v/v; Flow rate; 17 mL/min; DAD detection 220 Loop 1000 μL; Total amount: 80 mg. Solubilization: 80 mg in 4 mL DCM/(EtOH/MeOH 1/1) 1/1=20 mg/mL. Injection: 20 mg/injection.

Liquid Chromatography—Mass spectrometry Method DP

HPLC conditions: Chiralpak IC (25*2.0 cm, 5 um); Mobile phase: n-hexane/(EtOH+0.1% isopropylamine) 75/25% v/v; Flow rate; 17 mL/min; DAD detection 220 nm Loop 700 μL; Total amount: 35 mg. Solubilization: 35 mg in 2.5 mL DCM/(EtOH/MeOH 1/1) 1/1=14 mg/mL. Injection: 9.8 mg/injection.

Liquid Chromatography—Mass spectrometry Method DQ

HPLC conditions: Chiralpak AS-H (25*2.0 cm, 5 um); Mobile phase: n-hexane/(EtOH+0.1% isopropylamine) 70/30% v/v; Flow rate; 17 mL/min; DAD detection 220 nm, Loop 750 μL; Total amount: 30 mg. Solubilization: 30 mg in 1.8 mL EtOH/MeOH 1/1=16.7 mg/mL. Injection: 12.5 mg/injection.

Liquid Chromatography—Mass spectrometry Method DR

HPLC conditions: Chiralpak IC (25*2.0 cm, 5 um); Mobile phase: n-hexane/(EtOH+0.1% isopropylamine) 70/30% v/v; Flow rate; 17 mL/min; DAD detection 220 nm Loop 500 μL Total amount: 30 mg. Solubilization: 30 mg in 2.3 mL EtOH/MeOH 1/1=13.0 mg/mL. Injection: 6.5 mg/injection.

Liquid Chromatography—Mass spectrometry Method DS

HPLC conditions: Chiralpak IC (25*2.0 cm, 5 um); Mobile phase: n-hexane/(EtOH+0.1% isopropylamine) 60/40% v/v; Flow rate; 17 mL/min; DAD detection 220 nm Loop 1500 μL Total amount: 56 mg. Solubilization: 56 mg in 5.0 mL EtOH/MeOH 1/1=11.2 mg/mL. Injection: 16.8 mg/injection.

Liquid Chromatography—Mass spectrometry Method DT

HPLC conditions: Chiralpak IC (25*2.0 cm, 5 um); Mobile phase: n-hexane/(EtOH+0.1% isopropylamine) 60/40% v/v; Flow rate; 17 mL/min; DAD detection 220 nm Loop 500 μL; Total amount: 56 mg. Solubilization: 56 mg in 3.0 mL EtOH/MeOH 1/1=18.7 mg/mL. Injection: 9.3 mg/injection.

Liquid Chromatography—Mass spectrometry Method DU

HPLC conditions: Chiralpak IC (25*2.0 cm, 5 um); Mobile phase: n-hexane/(EtOH+0.1% isopropylamine) 70/30% v/v; Flow rate; 17 mL/min; DAD detection 220 Loop 1000 μL; Total amount: 23 mg. Solubilization: 23 mg in 4.0 mL DCM/(EtOH/MeOH 1/1) 1/1=5.75 mg/mL. Injection: 5.75 mg/injection.

Liquid Chromatography—Mass spectrometry Method DV

HPLC conditions: Chiralpak AS-H (25*2.0 cm, 5 um); Mobile phase: n-hexane/(EtOH+0.1% isopropylamine) 70/30% v/v; Flow rate; 17 mL/min; DAD detection 220 nm Loop 1500 μL; Total amount: 32 mg. Solubilization: 32 mg in 4.2 mL 1,1,1,3,3,3-hexafluoro2-propanol/(EtOH/MeOH 1/1) 1/1=7.6 mg/mL. Injection: 11.4 mg/injection.

Liquid Chromatography—Mass spectrometry Method DW

HPLC conditions: Chiralpak AD-H (25*2.0 cm, 5 um); Mobile phase: n-hexane/(EtOH+0.1% isopropylamine) 60/40% v/v; Flow rate; 17 mL/min; DAD detection 220 nm Loop 1000 μL; Total amount: 38 mg. Solubilization: 38 mg in 5.7 mL EtOH/MeOH 1/1=6.7 mg/mL. Injection: 6.7 mg/injection.

Liquid Chromatography—Mass spectrometry Method DX

HPLC conditions: Chiralpak AS-H (25*2.0 cm, 5 um); Mobile phase: n-hexane/(EtOH+0.1% isopropylamine) 70/30% v/v; Flow rate; 17 mL/min; DAD detection 220 nm Loop 1000 μL; Total amount: 40 mg. Solubilization: 40 mg in 4.0 mL EtOH/MeOH 1/1=10 mg/mL. Injection: 10 mg/injection.

Liquid Chromatography—Mass spectrometry Method DY

HPLC conditions: Chiralpak AS-H (25*2.0 cm, 5 um); Mobile phase: n-hexane/(EtOH+0.1% isopropylamine) 80/20% v/v; Flow rate; 17 mL/min; DAD detection 220 nm Loop 700 μL; Total amount: 80 mg. Solubilization: 80 mg in 5.5 mL EtOH/MeOH 1/1=14.5 mg/mL. Injection: 10.2 mg/injection.

Liquid Chromatography—Mass spectrometry Method DZ

HPLC conditions: Chiralpak AD-H (25*2.0 cm, 5 um); Mobile phase: n-hexane/(EtOH+0.1% isopropylamine) 50/50% v/v; Flow rate; 17 mL/min; DAD detection 220 nm Loop 1000 μL; Total amount: 88 mg. Solubilization: 88 mg in 6.0 mL (EtOH/MeOH 1/1)=14.7 mg/mL. Injection: 14.7 mg/injection.

Liquid Chromatography—Mass spectrometry Method EA

HPLC conditions: Chiralpak AD-H (25*2.0 cm, 5 um); Mobile phase: n-hexane/(EtOH+0.1% isopropylamine) 60/40% v/v; Flow rate; 17 mL/min; DAD detection 220 nm Loop 700 μL; Total amount: 90 mg. Solubilization: 90 mg in 7.5 mL (EtOH/MeOH 1/1)=12 mg/mL. Injection: 8.4 mg/injection.

Liquid Chromatography—Mass spectrometry Method EB

HPLC conditions: Chiralcel OJ-H (25*2.0 cm, 5 um); Mobile phase: n-hexane/(EtOH/MeOH 1/1+0.1% isopropylamine) 60/40% v/v; Flow rate; 17 mL/min; DAD detection 220 nm Loop 750 μL; Total amount: 57 mg. Solubilization: 57 mg in 5.0 mL EtOH/MeOH 1/1=11.4 mg/mL. Injection: 8.5 mg/injection.

Liquid Chromatography—Mass spectrometry Method EC

HPLC conditions: Chiralpak AD-H (25*2.0 cm, 5 um); Mobile phase: n-hexane/(EtOH/MeOH 1/1+0.1% isopropylamine) 55/45% v/v; Flow rate; 17 mL/min; DAD detection 220 nm Loop 600 μL; Total amount: 59 mg. Solubilization: 59 mg in 5.0 mL 1,1,1,3,3,3-hexafluoro-2-propanol/(EtOH/MeOH 1/1) 1/1 11.8 mg/mL. Injection: 7.1 mg/injection.

Liquid Chromatography—Mass spectrometry Method ED

HPLC conditions: Phenomenex Gemini-NX C18 (75*30 mm, 3 um); Mobile phase: [water (0.05% NH₄OH+10 mM NH₄HCO₃)-ACN]; B %: 25%-45%.

Liquid Chromatography—Mass spectrometry Method EE

SFC conditions: DAICEL CHIRALPAK IG (250*30 mm, 10 um); Mobile phase: [0.1% NH₄OH, MeOH]; B %: 35%.

Liquid Chromatography—Mass spectrometry Method EF

HPLC conditions: Waters Xbridge Prep OBD C18 (150*40 mm*10 um); Mobile phase: [water (10 mM NH₄HCO₃)-ACN]; B %: 40%-70%.

Liquid Chromatography—Mass spectrometry Method EG

SFC conditions: DAICEL CHIRALPAK AD (250*30 mm, 10 um); Mobile phase: [0.1% NH₄OH, EtOH]; B %: 50%.

Liquid Chromatography—Mass spectrometry Method EH

HPLC conditions: Phenomenex luna C18 (100*40 mm, 5 um); Mobile phase: [water (0.1% TFA)-ACN]; B %: 15%-48%.

Liquid Chromatography—Mass spectrometry Method EI

SFC conditions: DAICEL CHIRALPAK IG (250*30 mm, 10 um); Mobile phase: [0.1% NH₄OH, MeOH]; B %: 55%.

Liquid Chromatography—Mass spectrometry Method EJ

HPLC conditions: Phenomenex luna C18 (100*40 mm, 5 um); Mobile phase: [water (0.1% TFA)-ACN]; B %: 20%-57%.

Liquid Chromatography—Mass spectrometry Method EK

SFC conditions: REGIS (s,s) WHELK-01 (250*50 mm, 10 um); Mobile phase: [0.1% NH₄OH, MeOH]; B %: 45%.

Liquid Chromatography—Mass spectrometry Method EL

HPLC conditions: Phenomenex Gemini-NX (150*30 mm, 5 um); Mobile phase: [water (0.1% TFA)-ACN]; B %: 40%-70%.

Liquid Chromatography—Mass spectrometry Method EM

HPLC conditions: Phenomenex Gemini-NX (150*30 mm, 5 um); Mobile phase: [water (0.1% TFA)-ACN]; B %: 40%-70%.

Liquid Chromatography—Mass spectrometry Method EN

HPLC conditions: Phenomenex Gemini-NX (150*30 mm, 5 um); Mobile phase: [water (0.1% TFA)-ACN]; B %: 15%-45%.

Liquid Chromatography—Mass spectrometry Method EO

SFC conditions: DAICEL CHIRALPAK AD (250*30 mm, 10 um); Mobile phase: [0.1% NH₄OH, ETOH]; B %: 60%.

Liquid Chromatography—Mass spectrometry Method EP

HPLC conditions: Phenomenex Gemini-NX C18 (75*30 mm, 3 um); Mobile phase: [water (10 mM NH₄HCO₃)-ACN]; B %: 20%-45%.

Liquid Chromatography—Mass spectrometry Method EQ

SFC conditions: REGIS (s,s) WHELK-O1 (250*30 mm, 5 um); Mobile phase: [0.1% NH₄OH, EtOH]; B %: 45%.

Liquid Chromatography—Mass spectrometry Method ER

HPLC conditions: Phenomenex luna C18 (100*40 mm, 5 um); Mobile phase: [water (0.1% TFA)-ACN]; B %: 20%-60%.

Liquid Chromatography—Mass spectrometry Method ES

SFC conditions: DAICEL CHIRALPAK IG (250*30 mm, 10 um); Mobile phase: [0.1% NH₄OH, EtOH]; B %: 50%.

Liquid Chromatography—Mass spectrometry Method ET

HPLC conditions: Kromasil C18 (250*50 mm, 10 um); Mobile phase: [water (10 mM NH₄HCO₃)-ACN]; B %: 35%-75%.

Liquid Chromatography—Mass spectrometry Method EU

HPLC conditions: Mobile phase: [water (0.1% TFA)-ACN]; B %: 15%-45%.

Liquid Chromatography—Mass spectrometry Method EV

SFC conditions: DAICEL CHIRALPAK AD (250*30 mm, 10 um); Mobile phase: [0.1% NH₄OH, EtOH]; B %: 55%.

Liquid Chromatography—Mass spectrometry Method EW

HPLC conditions: Phenomenex luna C18 (100*40 mm, 5 um); Mobile phase: [water (0.1% TFA)-ACN]; B %: 10%-45%.

Liquid Chromatography—Mass spectrometry Method EX

SFC conditions: DAICEL CHIRALPAK IG (250*30 mm, 10 um); Mobile phase: [0.1% NH₄OH, EtOH]; B %: 55%.

Liquid Chromatography—Mass spectrometry Method EY

HPLC conditions: Waters Xbridge BEH C18 (100*30 mm, 10 um); Mobile phase: [water (10 mM NH₄HCO₃)-ACN]; B %: 20%-50%.

Liquid Chromatography—Mass spectrometry Method EZ

SFC conditions: DAICEL CHIRALPAK AD (250*30 mm, 10 um); Mobile phase: [0.1% NH₄OH, EtOH]; B %: 60%.

Liquid Chromatography—Mass spectrometry Method FA

HPLC conditions: Waters Xbridge BEH C18 (100*30 mm, 10 um); Mobile phase: [water (10 mM NH₄HCO₃)-ACN]; B %: 20%-50%.

Liquid Chromatography—Mass spectrometry Method FB

SFC conditions: REGIS (S,S) WHELK-01 (250*25 mm, 10 um); Mobile phase: [0.1% NH₄OH, IPA]; B %: 50%.

Liquid Chromatography—Mass spectrometry Method FC

HPLC conditions: Waters Xbridge BEH C18 (100*30 mm, 10 um); Mobile phase: [water (10 mM NH₄HCO₃)-ACN]; B %: 20%-55%.

Liquid Chromatography—Mass spectrometry Method FD

HPLC conditions: Phenomenex Gemini-NX (80*40 mm, 3 um); Mobile phase: [water (10 mM NH₄HCO₃)-ACN]; B %: 20%-50%.

Liquid Chromatography—Mass spectrometry Method FE

SFC conditions: REGIS (R,R)WHELK-01 (250*25 mm, 10 um); Mobile phase: [0.1% NH₄OH, EtOH]; B %: 50%.

Liquid Chromatography—Mass spectrometry Method FF

HPLC conditions: Waters Xbridge BEH C18 (100*30 mm, 10 um); Mobile phase: [water (0.1% TFA)-ACN]; B %: 6%-40%.

Liquid Chromatography—Mass spectrometry Method FG

SFC conditions: DAICEL CHIRALCEL OJ (250*30 mm, 10 um); Mobile phase: [0.1% NH₄OH, EtOH]; B %: 50%.

Liquid Chromatography—Mass spectrometry Method FH

SFC conditions: DAICEL CHIRALPAK AD (250*30 mm, 10 um); Mobile phase: [EtOH]; B %: 55%.

Liquid Chromatography—Mass spectrometry Method FI

HPLC conditions: Phenomenex Gemini-NX C18 (75*30 mm*3 um); Mobile phase: [water (10 mM NH₄HCO₃)-ACN]; B %: 25%-55%.

Liquid Chromatography—Mass spectrometry Method FJ

HPLC conditions: Phenomenex luna C18 (100*40 mm, 5 um); Mobile phase: [water (0.1% TFA)-ACN]; B %: 20%-60%.

Liquid Chromatography—Mass spectrometry Method FK

SFC conditions: DAICEL CHIRALCEL OD (250*30 mm, 10 um); Mobile phase: [0.1% NH₄OH, IPA]; B %: 40%.

Liquid Chromatography—Mass spectrometry Method FL

HPLC conditions: Waters Xbridge BEH C18 (100*30 mm, 10 um); Mobile phase: [water (10 mM NH₄HCO₃)-ACN]; B %: 35%-65%.

Liquid Chromatography—Mass spectrometry Method FM

SFC conditions: DAICEL CHIRALPAK AD (250*30 mm, 10 um); Mobile phase: [0.1% NH₄OH, MeOH]; B %: 25%.

Liquid Chromatography—Mass spectrometry Method FN

HPLC conditions: Phenomenex luna C18 (100*40 mm, 5 um); Mobile phase: [water (0.1% TFA)-ACN]; B %: 35%-65%.

Liquid Chromatography—Mass spectrometry Method FO

SFC conditions: DAICEL CHIRALPAK AS (250*30 mm, 10 um); Mobile phase: [0.1% NH₄OH, EtOH]; B %: 45%.

Liquid Chromatography—Mass spectrometry Method FP

HPLC conditions: Phenomenex luna C18 (100*40 mm, 5 um); Mobile phase: [water (0.1% TFA)-ACN]; B %: 5%-35%.

Liquid Chromatography—Mass spectrometry Method FQ

SFC conditions: DAICEL CHIRALPAK AS (250*30 mm, 10 um); Mobile phase: [0.1% NH₄OH, MeOH]; B %: 45%.

Liquid Chromatography—Mass spectrometry Method FR

HPLC conditions: Phenomenex Luna C18 (150*30 mm, 5 um); Mobile phase: [water (0.1% TFA)-ACN]; B %: 38%-68%.

Liquid Chromatography—Mass spectrometry Method FS

HPLC conditions: Waters Xbridge BEH C18 (100*30 mm, 10 um); Mobile phase: [water (10 mM NH₄HCO₃)-ACN]; B %: 23%-53%.

Liquid Chromatography—Mass spectrometry Method FT

SFC conditions: DAICEL CHIRALPAK IG (250*30 mm, 10 um); Mobile phase: [0.1% NH₄OH, MeOH]; B %: 55%.

Liquid Chromatography—Mass spectrometry Method FU

SFC conditions: DAICEL CHIRALPAK AD (250*30 mm, 10 um); Mobile phase: [0.1% NH₄OH, EtOH]; B %: 55%.

Liquid Chromatography—Mass spectrometry Method FV

HPLC conditions: Waters Xbridge Prep OBD C18 (150*40 mm, 10 um); Mobile phase: [water (10 mM NH₄HCO₃)-ACN]; B %:25%-55%.

Liquid Chromatography—Mass spectrometry Method FW

SFC conditions: DAICEL CHIRALPAK AD (250*30 mm, 10 um); Mobile phase: [0.1% NH₄OH, EtOH]; B %: 50%.

Liquid Chromatography—Mass spectrometry Method FX

HPLC conditions: Waters Xbridge BEH C18 (100*30 mm, 10 um); Mobile phase: [water (10 mM NH₄HCO₃)-ACN]; B %: 30%-50%.

Liquid Chromatography—Mass spectrometry Method FY

HPLC conditions: Waters Xbridge BEH C18 (100*30 mm, 10 um); Mobile phase: [water (10 mM NH₄HCO₃)-ACN]; B %: 22%-42%.

Liquid Chromatography—Mass spectrometry Method FZ

SFC conditions: REGIS (R,R)WHELK-01 (250*25 mm, 10 um); Mobile phase: [0.1% NH₄OH, EtOH]; B %: 50%.

Liquid Chromatography—Mass spectrometry Method GA

HPLC conditions: Phenomenex Gemini-NX (80*40 mm, 3 um); Mobile phase: [water (10 mM NH₄HCO₃)-ACN]; B %: 40%-70%.

Liquid Chromatography—Mass spectrometry Method GB

HPLC conditions: Waters Xbridge BEH C18 (100*25 mm, 5 um); Mobile phase: [water (10 mM NH₄HCO₃)-ACN]; B %: 30%-65%.

Liquid Chromatography—Mass spectrometry Method GC

SFC conditions: DAICEL CHIRALPAK AS (250*30 mm, 10 um); Mobile phase: [0.1% NH₄OH, IPA]; B %: 30%.

Liquid Chromatography—Mass spectrometry Method GD

HPLC conditions: Waters Xbridge Prep OBD C18 (150*40 mm, 10 um); Mobile phase: [water (10 mM NH₄HCO₃)-ACN]; B %: 25%-55%.

Liquid Chromatography—Mass spectrometry Method GE

HPLC conditions: Phenomenex Gemini-NX C18 (75*30 mm, 3 um); Mobile phase: [water (0.05% NH₄OH+10 mM NH₄HCO₃)-ACN]; B %: 20%-45%.

Liquid Chromatography—Mass spectrometry Method GF

SFC conditions: REGIS (R,R)WHELK-01 (250*25 mm, 10 um); Mobile phase: [0.1% NH₄OH, EtOH]; B %: 50%.

Liquid Chromatography—Mass spectrometry Method GH

HPLC conditions: Waters Xbridge BEH C18 (100*30 mm, 10 um); Mobile phase: [water (10 mM NH₄HCO₃)-ACN]; B %: 15%-45%.

Liquid Chromatography—Mass spectrometry Method GI

SFC conditions: DAICEL CHIRALPAK IG (250*30 mm, 10 um); Mobile phase: [0.1% NH₄OH, EtOH]; B %: 62%.

Liquid Chromatography—Mass spectrometry Method GJ

HPLC conditions: Column Chiralpak IC (25*2.0 cm, 5 μm); Mobile phase n-hexane/(EtOH+0.1% isopropylamine) 60/40% v/v; Flow rate: 17 mL/min; DAD detection 220 Loop 1000 μL; Total amount: 13 mg; Solubilization: 13 mg in 2.0 mL EtOH/MeOH 1/1=6.5 mg/mL; Injection: 6.5 mg/injection.

Liquid Chromatography—Mass spectrometry Method GK

HPLC conditions: Column Chiralpak AD-H (25*2.0 cm, 5 μm); Mobile phase n-hexane/(EtOH/MeOH+0.1% isopropylamine) 60/40% v/v; Flow rate: 17 mL/min; DAD detection 220 nm Loop 3000 μL; Total amount 87 mg; Solubilization: 87 mg in 11 mL (4 mL hexafluoro-2-propanol+6 mL EtOH/MeOH 1/1)=7.9 mg/mL; Injection: 14.4 mg/injection.

Liquid Chromatography—Mass spectrometry Method GL

HPLC conditions: Column Chiralpak AD-H (25*2.0 cm, 5 μm); Mobile phase n-hexane/(EtOH/MeOH+0.10% isopropylamine) 60/40% v/v; Flow rate: 17 mL/min DAD detection 220 nm Loop 2000 μL; Total amount 25 mg; Solubilization: 25 mg in 4 mL (EtOH/MeOH 1/1)=6.3 mg/mL; Injection: 12.5 mg/injection.

Liquid Chromatography—Mass spectrometry Method GM

HPLC conditions: Column Chiralcel OJ-H (25*2.0 cm, 5 μm); Mobile phase n-hexane/EtOH 60/40% v/v; Flow rate: 17 mL/min DAD detection 220 nm Loop 1200 μL; Total amount: 29 mg; Solubilization: 29 mg in 5.0 mL 1,1,1,3,3,3-hexafluoro-2-propanol/(EtOH/MeOH 1/1) 1/1=5.8 mg/mL; Injection: 7.0 mg/injection.

Liquid Chromatography—Mass spectrometry Method GN

HPLC conditions: Column Chiralpak AD-H (25*2.0 cm, 5 μm); Mobile phase n-hexane/(EtOH+0.1% isopropylamine) 70/30% v/v; Flow rate: 17 mL/min; DAD detection 220 nm Loop 1200 μL; Total amount: 15 mg; Solubilization: 15 mg in 3.5 mL EtOH/MeOH 1/1=4.3 mg/mL; Injection: 5.1 mg/injection.

Liquid Chromatography—Mass spectrometry Method GO

HPLC conditions: Column Chiralpak AD-H (25*2.0 cm, 5 μm); Mobile phase n-hexane/(EtOH+0.1% isopropylamine) 70/30% v/v; Flow rate: 17 mL/min; DAD detection 220 nm Loop 500 μL Total amount: 100 mg; Solubilization 100 mg in 5.0 mL EtOH/MeOH 1/1=20 mg/mL; Injection 10 mg/injection.

Liquid Chromatography—Mass spectrometry Method GP

HPLC conditions: Column Chiralpak AS-H (25*2.0 cm, 5 μm); Mobile phase n-hexane/(EtOH+0.1% isopropylamine) 60/40% v/v; Flow rate: 17 mL/min DAD detection 220 nm Loop 1500 μL Total amount: 69 mg; Solubilization: 69 mg in 7.0 mL 1,1,1,3,3,3-hexafluoro-2-propanol/(EtOH/MeOH 1/1) 1/1=9.8 mg/mL; Injection 14.8 mg/injection.

Liquid Chromatography—Mass spectrometry Method GQ

HPLC conditions: Column Chiralpak AD-H (25*2.0 cm, 5 μm); Mobile phase n-hexane/(EtOH+0.1% isopropylamine) 65/35% v/v; Flow rate: 17 mL/min; DAD detection 220 nm Loop 2000 μL; Total amount: 24 mg; Solubilization: 24 mg in 8.0 mL 1,1,1,3,3,3-hexafluoro-2-propanol/(EtOH/MeOH 1/1) 1/1=3 mg/mL; Injection: 6 mg/injection.

Liquid Chromatography—Mass spectrometry Method GR

HPLC conditions: Column Chiralpak AS-H (25*2.0 cm, 5 μm); Mobile phase n-hexane/(EtOH+0.1% isopropylamine) 60/40% v/v; Flow rate: 17 mL/min; DAD detection 220 nm Loop 1000 μL; Total amount: 3.5 mg Solubilization: 3.5 mg in 1.0 mL EtOH/MeOH 1/1=3.5 mg/mL; Injection: 3.5 mg/injection.

Liquid Chromatography—Mass spectrometry Method GS

HPLC conditions: Column Chiralpak IC (25*2.0 cm, 5 μm); Mobile phase n-hexane/(EtOH+0.1% isopropylamine) 65/35% v/v; Flow rate: 17 mL/min; DAD detection 220 nm Loop 1000 μL; Total amount: 10 mg Solubilization: 10 mg in 2.0 mL EtOH/MeOH 1/1=5 mg/mL; Injection: 5 mg/injection.

Liquid Chromatography—Mass spectrometry Method GT

HPLC conditions: Column Chiralcel OJ-H (25*2.0 cm, 5 μm); Mobile phase: n-hexane/(EtOH+0.1% isopropylamine) 65/35% v/v; Flow rate: 17 mL/min; DAD detection 220 nm Loop 750 μL; Total amount: 58 mg; Solubilization: 58 mg in 4.0 mL EtOH/MeOH 1/1=14.5 mg/mL; Injection: 10.9 mg/injection.

Liquid Chromatography—Mass spectrometry Method GU

HPLC conditions: Column Chiralpak AD-H (25*2.0 cm, 5 μm); Mobile phase: n-hexane/(EtOH+0.1% isopropylamine) 70/30% v/v; Flow rate: 17 mL/min; DAD detection 220 nm Loop 750 μL; Total amount: 30.7 mg; Solubilization: 30.7 mg in 3.0 mL EtOH/MeOH 1/1=10.2 mg/mL; Injection: 7.7 mg/injection.

Liquid Chromatography—Mass spectrometry Method GV

HPLC conditions: Column Chiralcel OD-H (25*2.0 cm, 5 μm); Mobile phase: n-hexane/(EtOH+0.1% isopropylamine) 46/54% v/v; Flow rate: 17 mL/min; DAD detection 220 nm Loop 1250 μL; Total amount: 10 mg; Solubilization: 10 mg in 2.5 mL EtOH/MeOH 1/1=4.0 mg/mL; Injection: 5.0 mg/injection.

Liquid Chromatography—Mass spectrometry Method GW

HPLC conditions: Column Chiralpak AS-H (25*2.0 cm, 5 μm); Mobile phase: n-hexane/(EtOH/MeOH 1/1+0.1% isopropylamine) 55/45% v/v; Flow rate: 17 mL/min; DAD detection 220 nm Loop 1000 μL; Total amount: 63 mg; Solubilization: 63 mg in 4.0 mL EtOH/MeOH 1/1=15.7 mg/mL; Injection: 15.7 mg/injection.

Liquid Chromatography—Mass spectrometry Method GX

HPLC conditions: Column Chiralpak AS-H (25*2.0 cm, 5 μm); Mobile phase: n-hexane/(EtOH+0.1% isopropylamine) 55/45% v/v; Flow rate: 17 mL/min; DAD detection 220 nm Loop 1500 μL; Total amount: 35 mg; Solubilization: 35 mg in 4.5 mL (hexafluoro-2-propanol/(EtOH/MeOH 1/1) 1/1=7.8 mg/mL; Injection: 11.7 mg/injection.

Liquid Chromatography—Mass spectrometry Method GY

HPLC conditions: Column Chiralpak AS-H (25*2.0 cm, 5 μm); Mobile phase: n-hexane/(EtOH+0.1% isopropylamine) 60/40% v/v; Flow rate: 17 mL/min; DAD detection 220 nm Loop 2500 μL; Total amount: 44 mg; Solubilization: 44 mg in 17 mL EtOH/MeOH 1/1=2.6 mg/mL; Injection: 6.5 mg/injection.

Liquid Chromatography—Mass spectrometry Method GZ

HPLC conditions: Column Chiralpak AS-H (25*2.0 cm, 5 μm); Mobile phase: n-hexane/(EtOH+0.1% isopropylamine) 80/20% v/v; Flow rate: 17 mL/min; DAD detection 220 nm Loop 1000 μL; Total amount: 38 mg; Solubilization: 38 mg in 9.0 mL EtOH/MeOH 1/1=4.2 mg/mL; Injection: 4.2 mg/injection.

Liquid Chromatography—Mass spectrometry Method HA

HPLC conditions: Column Chiralpak AD-H (25*2.0 cm, 5 μm); Mobile phase: n-hexane/(EtOH+0.1% isopropylamine) 60/40% v/v; Flow rate: 17 mL/min; DAD detection 220 nm Loop 1000 μL; Total amount: 52 mg; Solubilization: 52 mg in 5.0 mL EtOH/MeOH 1/1=10.4 mg/mL; Injection: 10.4 mg/injection.

Liquid Chromatography—Mass spectrometry Method HB

HPLC conditions: Column Chiralpak AD-H (25*2.0 cm, 5 μm); Mobile phase: n-hexane/(EtOH+0.1% isopropylamine) 70/30% v/v; Flow rate: 17 mL/min; DAD detection 220 nm Loop 3000 μL; Total amount: 25 mg; Solubilization: 25 mg in 3.0 mL EtOH/MeOH 1/1=8.3 mg/mL; Injection: 12.5 mg/injection.

Liquid Chromatography—Mass spectrometry Method HC

HPLC conditions: Column Chiralcel OD-H (25*2.0 cm, 5 μm); Mobile phase: n-hexane/(EtOH+0.1% isopropylamine) 46/54% v/v; Flow rate: 17 mL/min; DAD detection 220 nm Loop 1250 μL; Total amount: 10 mg; Solubilization: 10 mg in 2.5 mL EtOH/MeOH 1/1=4.0 mg/mL; Injection: 5.0 mg/injection.

Liquid Chromatography—Mass spectrometry Method HD

HPLC conditions: Column Whelk 01 (25*3.0 cm, 10 μm); Mobile phase: n-hexane/(EtOH+0.1% isopropylamine) 40/60% v/v; Flow rate: 40 mL/min; DAD detection 220 nm Loop 3000 μL; Total amount: 6 mg; Solubilization: 6 mg in 3 mL (hexafluoro-3-isopropanol/(EtOH/MeOH 1/1) 1/1=2.0 mg/mL Injection: 6 mg/injection.

Liquid Chromatography—Mass spectrometry Method HE

HPLC conditions: Column Chiralpak AS-H (25*2.0 cm, 5 μm); Mobile phase: n-hexane/(EtOH+0.1% isopropylamine) 60/40% v/v; Flow rate: 17 mL/min; DAD detection 220 nm Loop 2000 μL; Total amount: 2 mg; Solubilization: 2 mg in 2.0 mL 1,1,1,3,3,3-hexafluoro-2-propanol/(EtOH/MeOH 1/1) 1/1=1 mg/mL Injection: 2 mg/injection.

Liquid Chromatography—Mass spectrometry Method HF

HPLC conditions: Column Chiralpak AS-H (25*2.0 cm, 5 μm); Mobile phase: n-hexane/(EtOH+0.1% isopropylamine) 55/45% v/v; Flow rate: 17 mL/min; DAD detection 220 nm Loop 1500 μL; Total amount: 100 mg; Solubilization: 100 mg in 6.5 mL 1,1,1,3,3,3-hexafluoro-2-propanol/(EtOH/MeOH 1/1) 1/1=15.4 mg/mL; Injection: 23.1 mg/injection.

Liquid Chromatography—Mass spectrometry Method HG

HPLC conditions: Column Chiralpak AS-H (25*2.0 cm, 5 μm); Mobile phase: n-hexane/(EtOH+0.1% isopropylamine) 55/45% v/v; Flow rate: 17 mL/min; DAD detection 220 nm Loop 2000 μL; Total amount: 87 mg; Solubilization: 87 mg in 10 mL 1,1,1,3,3,3-hexafluoro-2-propanol/(EtOH/MeOH 1/1) 1/1=8.7 mg/mL; Injection: 17.4 mg/injection.

Liquid Chromatography—Mass spectrometry Method HH

HPLC conditions: Column Chiralpak AS-H (25*2.0 cm, 5 μm); Mobile phase: n-hexane/(EtOH+0.1% isopropylamine) 55/45% v/v; Flow rate: 17 mL/min; DAD detection 220 nm Loop 2500 μL; Total amount: 15 mg; Solubilization: 15 mg in 7.0 mL 1,1,1,3,3,3-hexafluoro-2-propanol/(EtOH/MeOH 1/1) 1/1=2.1 mg/mL; Injection: 5.3 mg/injection.

Liquid Chromatography—Mass spectrometry Method HI

HPLC conditions: Column Chiralpak AS-H (25*2.0 cm, 5 μm); Mobile phase: n-hexane/(EtOH+0.1% isopropylamine) 55/45% v/v; Flow rate: 17 mL/min; DAD detection 220 nm Loop 1500 μL; Total amount: 125 mg; Solubilization: 125 mg in 6.0 mL 1,1,1,3,3,3-hexafluoro-2-propanol/(EtOH/MeOH 1/1) 1/1=20.8 mg/mL; Injection: 30.2 mg/injection.

Liquid Chromatography—Mass spectrometry Method HJ

HPLC conditions: Column Chiralpak AD-H (25*2.0 cm, 5 μm); Mobile phase: n-hexane/(EtOH+0.1% isopropylamine) 60/40% v/v; Flow rate: 17 mL/min; DAD detection 220 nm Loop 1000 μL; Total amount: 16 mg; Solubilization: 16 mg in 3.0 mL EtOH/MeOH 1/1=5.3 mg/mL; Injection: 5.3 mg/injection.

Liquid Chromatography—Mass spectrometry Method HK

HPLC conditions: Column Chiralpak AS-H (25*2.0 cm, 5 μm); Mobile phase: n-hexane/(EtOH+0.1% isopropylamine) 60/40% v/v; Flow rate: 17 mL/min; DAD detection 220 nm Loop 1000 μL; Total amount: 44 mg; Solubilization: 44 mg in 4.0 mL 1,1,1,3,3,3-hexafluoro-2-propanol/(EtOH/MeOH 1/1) 1/1=11 mg/mL; Injection: 11 mg/injection.

Liquid Chromatography—Mass spectrometry Method HL

SFC conditions: Column: Chiralpak AS, (250*30 mm, 10 um); mobile phase: A for CO₂ and B for MeOH(0.1% NH₃.H₂O); gradient: B %=33% isocratic elution mode; Flow rate: 60 g/min.

### Intermediate 1: tert-Butyl 5-((3-cyano-5,6,7,8-tetrahydroquinolin-8-yl)oxy)-3-iodo-1H-indazole-1-carboxylate

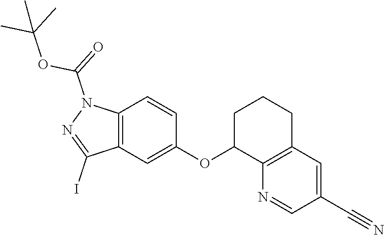 [SMILES: CC(C)(C)OC(=O)n1nc(I)c2cc(OC3CCCc4cc(C#N)cnc43)ccc21]

### Step 1: 8-Hydroxy-5,6,7,8-tetrahydroquinoline-3-carbonitrile

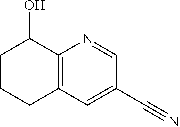 [SMILES: N#Cc1cnc2c(c1)CCCC2O]

A flask was charged with 8-oxo-5,6,7,8-tetrahydroquinoline-3-carbonitrile (100.57 mg, 0.580 mmol) and MeOH (10 mL). NaBH₄ (44.19 mg, 1.17 mmol) was added and the solution was stirred for 30 min. The reaction mixture was transferred to a separatory funnel with water and the aqueous layer was extracted with DCM (3×). The combined organic layers were dried over Na₂SO₄, filtered, and concentrated under vacuum. The material was purified by silica gel chromatography using a gradient of 0-50% EtOAc in cyclohexane (12 CV) as eluent to afford the title compound (88 mg, 86%). MS-ESI (m/z) calc'd for C₁₀H₁₁N₂O [M+H]⁺: 175.1. Found 175.1.

### Step 2: tert-Butyl 5-((3-cyano-5,6,7,8-tetrahydroquinolin-8-yl)oxy)-3-iodo-1H-indazole-1-carboxylate

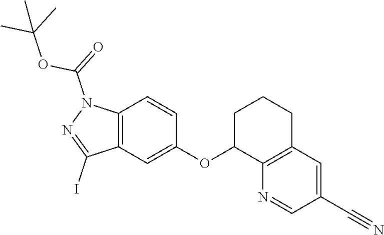 [SMILES: CC(C)(C)OC(=O)n1nc(I)c2cc(OC3CCCc4cc(C#N)cnc43)ccc21]

8-Hydroxy-5,6,7,8-tetrahydroquinoline-3-carbonitrile (85.0 mg, 0.490 mmol) was dissolved in THF (0.600 mL) and purged with N₂. tert-Butyl 5-hydroxy-3-iodo-1H-indazole-1-carboxylate (175.73 mg, 0.490 mmol) and triphenylphosphine (140.78 mg, 0.540 mmol) were added followed by dropwise addition of diethyl azodicarboxylate (84.98 mg, 0.490 mmol). After stirring for 1 hr, the solution was diluted with water and EtOAc. The organic layers were combined, washed with brine, dried over anhydrous Na₂SO₄, filtered and concentrated. This material was purified by silica gel chromatography using a gradient of 0-20% EtOAc in cyclohexane (12 CV) as eluent to afford the title compound (211 mg, 84%). MS-ESI (m/z) calc'd for C₂₂H₂₂IN₄O₃ [M+H]⁺: 517.1. Found 517.3.

### Intermediate 2: 5-Hydroxy-1-methoxy-5,6,7,8-tetrahydronaphthalene-2-carbonitrile

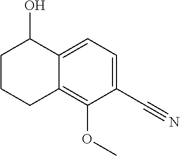

### Step 1: 6-Bromo-5-hydroxy-3,4-dihydronaphthalen-1(2H)-one

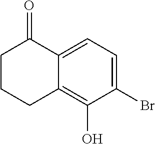 [SMILES: O=C1CCCc2c1ccc(Br)c2O]

To a solution of 5-hydroxy-1-tetralone (2.0 g, 12.33 mmol) in dry DCM (100 mL) under an N₂ atmosphere was added N-ethylethanamine (0.15 mL, 1.48 mmol). Then a solution of 1-bromopyrrolidine-2,5-dione (2.19 g, 12.33 mmol) in DCM (100 mL) was slowly added over 2 hrs at r.t. The reaction mixture was then stirred at r.t. for 1 hr. The solvent was evaporated under reduced pressure and the residue was purified by chromatography on a 100 g silica gel column using a 0-25% EtOAc/cyclohexane gradient eluent to afford the title compound (2.21 g, 74%) as a white solid. ¹H NMR (400 MHz, DMSO-d₆) δ 9.51 (br. s., 1H) 7.51 (d, J=8.36 Hz, 1H) 7.33 (d, J=8.58 Hz, 1H) 2.88 (t, J=6.05 Hz, 2H) 2.53-2.61 (m, 2H) 2.03 (quin, J=6.38 Hz, 2H). MS-ESI (m/z) calc'd for C₁₀H₁₀BrO₂ [M+H]⁺: 241.0. Found 241.0, 243.0.

### Step 2: 6-Bromo-5-methoxy-3,4-dihydronaphthalen-1(2H)-one

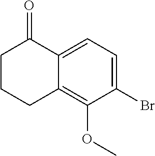

To a solution of 6-bromo-5-hydroxy-3,4-dihydro-2H-naphthalen-1-one (1.0 g, 4.15 mmol) in anhydrous DMF (7 mL) was added cesium carbonate (2027.25 mg, 6.22 mmol) followed by iodomethane (0.39 mL, 6.22 mmol). The mixture was stirred for 2 hrs at 25° C. Saturated aqueous NH₄Cl (10 mL) was added to quench the reaction, followed by addition of H₂O. The mixture was extracted with EtOAc (3×) and the combined organic phases were washed with brine (1×), dried over anhydrous Na₂SO₄ and the solvent was evaporated under reduced pressure to afford the title compound (830 mg, 78%) as a yellow solid. ¹H NMR (400 MHz, DMSO-d₆) δ 7.61-7.66 (m, 1H) 7.57-7.61 (m, 1H) 3.79 (s, 3H) 2.98 (t, J=6.05 Hz, 2H) 2.60 (dd, J=7.37, 5.83 Hz, 2H) 2.05 (quin, J=6.33 Hz, 2H). MS-ESI (m/z) calc'd for C₁₁H₁₂BrO₂ [M+H]⁺: 255.0. Found 255.0, 257.0.

### Step 3: 1-Methoxy-5-oxo-5,6,7,8-tetrahydronaphthalene-2-carbonitrile

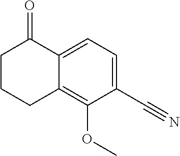

A 0.1 N aqueous solution of potassium hexacyanoferrate (II) (16.35 mL, 1.63 mmol), 6-bromo-5-methoxy-3,4-dihydro-2H-naphthalen-1-one (417.0 mg, 1.63 mmol) and KOAc (160.42 mg, 1.63 mmol) were dissolved in a mixture of 1,4-dioxane (20 mL)/H₂O (3 mL) in a sealed microwave vial. The mixture was degassed with N₂ for 15 minutes. Then XPhos (116.89 mg, 0.250 mmol) and XPhos Pd G3 (207.54 mg, 0.25 mmol) were added and the mixture was stirred at 100° C. overnight. XPhos (0.15 eq) and XPhos Pd G3 (0.15 eq) were added and the mixture was stirred at 100° C. for 2 hrs. This procedure was conducted a second time with 6-bromo-5-methoxy-3,4-dihydro-2H-naphthalen-1-one (400 mg, 1.54 mmol) and the mixtures were combined together. The reaction mixture was partitioned between H₂O and EtOAc, the phases were separated, the aqueous layer was extracted with EtOAc (3×), and the combined organic phases were washed with brine (2×), dried over anhydrous Na₂SO₄ and evaporated to dryness. The material was purified by chromatography on a 25 g silica gel column using a 0-50% EtOAc/cyclohexane gradient eluent to afford the title compound (520 mg, 81%) as an off-white solid. ¹H NMR (400 MHz, DMSO-d₆) δ 7.72-7.79 (m, 2H) 3.94-4.00 (m, 3H) 2.95 (t, J=6.16 Hz, 2H) 2.66 (dd, J=7.37, 5.83 Hz, 2H) 2.51 (dt, J=3.69, 1.79 Hz, 7H) 2.01-2.13 (m, 2H. MS-ESI (m/z) calc'd for C₁₂H₁₂NO₂ [M+H]⁺: 202.1. Found 202.1.

### Step 4: 5-Hydroxy-1-methoxy-5,6,7,8-tetrahydronaphthalene-2-carbonitrile

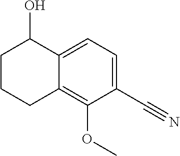

To a solution of 1-methoxy-5-oxo-7,8-dihydro-6H-naphthalene-2-carbonitrile (120.0 mg, 0.49 mmol) in MeOH (5 mL) was added sodium borohydride (37.0 mg, 0.98 mmol) and the mixture was stirred at 25° C. for 1 hr. A second equivalent of sodium borohydride was then added and the mixture was stirred for 1 hr. The solvent was evaporated and the residue was taken up in H₂O and extracted with DCM (3×). The combined organic layers were passed through a phase separator and evaporated to afford the title compound (98 mg, 99%) as a colorless oil. ¹H NMR (400 MHz, DMSO-d₆) δ 7.56 (d, J=8.14 Hz, 1H) 7.35 (d, J=7.92 Hz, 1H) 5.41 (d, J=5.94 Hz, 1H) 4.52-4.61 (m, 1H) 3.88 (s, 3H) 2.59-2.73 (m, 2H) 1.79-2.00 (m, 2H) 1.58-1.73 (m, 3H) MS-ESI (m/z) calc'd for C₁₂H₁₄NO₂ [M+H]⁺: 204.1. Found 204.0.

### Example 1: 1-((3-Methyl-1H-indazol-5-yl)oxy)-2,3-dihydro-1H-indene-5-carbonitrile, enantiomer 1 and 2

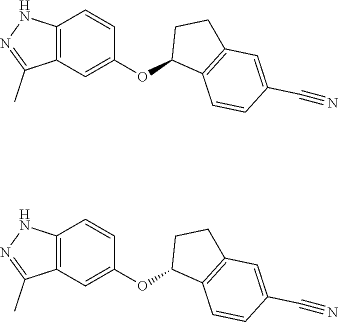 [SMILES: Cc1n[nH]c2ccc(O[C@H]3CCc4cc(C#N)ccc43)cc12 ; Cc1n[nH]c2ccc(O[C@@H]3CCc4cc(C#N)ccc43)cc12]

### Step 1: tert-Butyl 5-bromo-3-methyl-1H-indazole-1-carboxylate

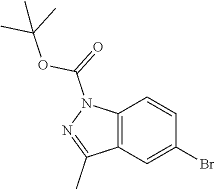 [SMILES: Cc1nn(C(=O)OC(C)(C)C)c2ccc(Br)cc12]

5-Bromo-3-methyl-1H-indazole (1.84 g, 8.72 mmol) and DMAP (11.0 mg, 0.090 mmol) were dissolved in DCM (36 mL). Di-tert-butyl dicarbonate (2.09 g, 9.59 mmol) was added, and the mixture was stirred at r.t. for 3 hrs. The solvent was removed under reduced pressure. The residue was diluted with EtOAc and washed with 1 N NaOH (2×), 0.1 N HCl solution, and brine. The organic layer was dried over Na₂SO₄ and filtered. The filtrate was concentrated to afford the title compound (2.72 g, 100%). ¹H NMR (400 MHz, CDCl₃) δ 8.02 (d, J=8.80 Hz), 7.76-7.86 (m, 1H), 7.59-7.66 (m, 1H), 2.52-2.65 (m, 3H), 1.71-1.74 (m, 9H). MS-ESI (m/z) calc'd for C₉H₈BrN₂O₂ [M+H]⁺: 255.0. Found [M−t-Bu+H]⁺ 255.2.

### Step 2: tert-Butyl 3-methyl-5-(4,4,5,5-tetramethyl-1,3,2-dioxaborolan-2-yl)-1H-indazole-1-carboxylate

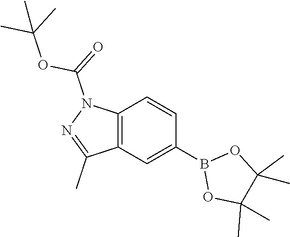 [SMILES: Cc1nn(C(=O)OC(C)(C)C)c2ccc(B3OC(C)(C)C(C)(C)O3)cc12]

To a solution of tert-butyl 5-bromo-3-methyl-1H-indazole-1-carboxylate (2.72 g, 8.73 mmol) in 1,4-dioxane (69 mL) was added [1,1′-bis(diphenylphosphino)ferrocene]dichloropalladium (II) (0.64 g, 0.870 mmol), bis(pinacolato)diborane (4.43 g, 17.45 mmol) and KOAc (1.71 g, 17.45 mmol). The reaction mixture was stirred at 80° C. for 2 hrs and the mixture was filtered and concentrated to afford the title compound (3.12 g, 100%) which was used without further purification. MS-ESI (m/z) calc'd for C₁₅H₂₀BN₂O₄ [M−t-Bu+H]⁺: 303.1. Found [M−t-Bu+H]⁺303.1.

### Step 3: tert-Butyl 5-hydroxy-3-methyl-1H-indazole-1-carboxylate

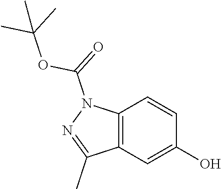 [SMILES: Cc1nn(C(=O)OC(C)(C)C)c2ccc(O)cc12]

To a solution of tert-butyl 3-methyl-5-(4,4,5,5-tetramethyl-1,3,2-dioxaborolan-2-yl)-1H-indazole-1-carboxylate (3.12 g, 8.73 mmol) in MeOH (25 mL) was added hydrogen peroxide (4.37 g, 45 mmol). The mixture was stirred at r.t. for 3 days. The reaction was quenched with an saturated aqueous Na₂SO₃ and then partitioned between water and EtOAc (3×). The combined organic phases were washed with brine, dried over anhydrous Na₂SO₄, filtered and concentrated. The residue was purified by column chromatography using a gradient of 0-50% EtOAc in cyclohexane (8 CV) as eluent to afford the title compound (2.2 g, 99%). ¹H NMR (400 MHz, CDCl₃) δ 7.99 (d, J=8.80 Hz, 1H), 7.10 (dd, J=9.02, 2.42 Hz, 1H), 7.05-6.99 (m, 1H), 2.60-2.49 (m, 3H), 1.74-1.72 (m, 9H). MS-ESI (m/z) calc'd for C₁₃H₁₇N₂O₃ [M+H]⁺: 249.1. Found 249.2.

### Step 4: 1-Hydroxy-2,3-dihydro-1H-indene-5-carbonitrile

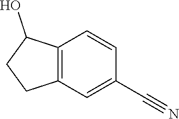 [SMILES: N#Cc1ccc2c(c1)CCC2O]

A flask was charged with the 2,3-dihydro-1-oxo-1H-indene-5-carbonitrile (500.0 mg, 3.18 mmol) and MeOH (10 mL). NaBH₄ (240.69 mg, 6.36 mmol) was then added to the stirred mixture and the resulting clear, colorless solution was left stirring for 30 min at r.t. The reaction mixture was diluted with water (100 mL) and extracted with DCM (3×100 mL). The combined organic layers were dried over Na₂SO₄, filtered and concentrated under vacuum to afford the title compound (498 mg, 98%) which was used without further purification. ¹H NMR (400 MHz, CDCl₃) δ 7.67-7.44 (m, 3H), 5.50-5.12 (m, 1H), 3.19-3.01 (m, 1H), 2.68-2.48 (m, 1H), 2.14-1.94 (m, 1H), 1.94-1.82 (m, 1H). MS-ESI (m/z) calc'd for C₁₀H₁₀NO [M+H]⁺: 160.1. Found 160.1.

### Step 5: tert-Butyl 5-((5-cyano-2,3-dihydro-1H-inden-1-yl)oxy)-3-methyl-1H-indazole-1-carboxylate

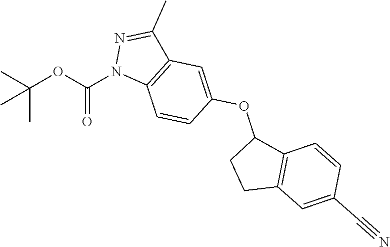 [SMILES: Cc1nn(C(=O)OC(C)(C)C)c2ccc(OC3CCc4cc(C#N)ccc43)cc12]

A solution of 1-hydroxy-2,3-dihydro-1H-indene-5-carbonitrile (150.0 mg, 0.940 mmol) in THF (1.007 mL) was purged with N₂. tert-Butyl 5-hydroxy-3-methylindazole-1-carboxylate (324.95 mg, 0.940 mmol) and triphenylphosphine (271.88 mg, 1.04 mmol) were added followed by dropwise addition of diethyl azodicarboxylate (164.12 mg, 0.940 mmol) at 0° C. and the reaction mixture was allowed to reach r.t. After stirring for 1 hr, the resulting solution was diluted with water and EtOAc. The organic layer was washed with brine, dried over anhydrous Na₂SO₄, filtered and concentrated. The material was purified by silica gel chromatography using a gradient of 0-20% EtOAc in cyclohexane (12 CV) as eluent to afford the title compound (110.7 mg, 30%). MS-ESI (m/z) calc'd for C₂₃H₂₄N₃O₃ [M+H]⁺: 390.2. Found 390.3.

### Step 6: 1-((3-Methyl-1H-indazol-5-yl)oxy)-2,3-dihydro-1H-indene-5-carbonitrile

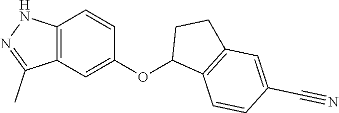 [SMILES: Cc1n[nH]c2ccc(OC3CCc4cc(C#N)ccc43)cc12]

To a solution of tert-butyl 5-((5-cyano-2,3-dihydro-1H-inden-1-yl)oxy)-3-methyl-1H-indazole-1-carboxylate (110.7 mg, 0.280 mmol) in DCM (2.5 mL) was added trifluoroacetic acid (0.5 mL, 6.53 mmol). The solution was stirred overnight, concentrated under reduced pressure and purified by reversed phase column chromatography using a gradient of 0-60% MeCN in H₂O (7 CV) as eluent to afford the title compound (33.1 mg, 40%). ¹H NMR (400 MHz, CDCl₃) δ 7.63 (s, 1H), 7.60-7.50 (M, 2H), 7.43-7.34 (m, 1H), 7.19 (d, J=2.20 Hz, 1H), 7.13 (s, 1H), 5.82 (dd, J=6.71, 5.17 Hz, 1H), 3.32-3.15 (M, 1H), 3.10-2.95 (m, 1H), 2.78-2.63 (m, 1H), 2.59 (s, 3H), 2.39-2.26 (m, 1H). MS-ESI (m/z) calc'd for C₁₈H₁₆N₃O [M+H]⁺: 290.1. Found 290.2.

### Step 7: 1-((3-Methyl-1H-indazol-5-yl)oxy)-2,3-dihydro-1H-indene-5-carbonitrile, enantiomer 1 and 2

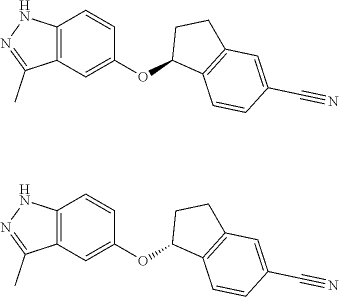 [SMILES: Cc1n[nH]c2ccc(O[C@H]3CCc4cc(C#N)ccc43)cc12 ; Cc1n[nH]c2ccc(O[C@@H]3CCc4cc(C#N)ccc43)cc12]

1-((3-Methyl-1H-indazol-5-yl)oxy)-2,3-dihydro-1H-indene-5-carbonitrile (33.1 mg, 0.114 mmol) was subjected to chiral separation using Method A to afford 1-((3-methyl-1H-indazol-5-yl)oxy)-2,3-dihydro-1H-indene-5-carbonitrile, enantiomer 1 (12.68 mg). ¹H NMR (400 MHz, DMSO-d₆) δ 12.48 (s, 1H), 7.82 (s, 1H), 7.69 (ddt, J=7.8, 1.5, 0.8 Hz, 1H), 7.57 (d, J=7.8 Hz, 1H), 7.40-7.27 (m, 2H), 7.03 (dd, J=8.9, 2.3 Hz, 1H), 5.93 (dd, J=6.8, 4.9 Hz, 1H), 3.15-2.87 (m, 2H), 2.64 (dddd, J=13.4, 8.3, 6.7, 5.1 Hz, 1H), 2.46 (s, 3H), 2.14-2.00 (m, 1H). MS-ESI (m/z) calc'd for C₁₈H₁₆N₃O [M+H]⁺: 290.1. Found 290.2. A second fraction was isolated to afford 1-((3-methyl-1H-indazol-5-yl)oxy)-2,3-dihydro-1H-indene-5-carbonitrile, enantiomer 2 (12.2 mg). ¹H NMR (400 MHz, DMSO-d₆) δ 12.49 (s, 1H), 7.83 (s, 1H), 7.70 (ddt, J=7.8, 1.5, 0.8 Hz, 1H), 7.57 (d, J=7.8 Hz, 1H), 7.43-7.24 (m, 2H), 7.04 (dd, J=8.9, 2.3 Hz, 1H), 5.98-5.84 (m, 1H), 3.15-3.02 (m, 1H), 2.95 (dt, J=16.1, 7.2 Hz, 1H), 2.65 (dddd, J=13.5, 8.4, 6.7, 5.2 Hz, 1H), 2.47 (s, 3H), 2.11 (dddd, J=13.5, 8.7, 6.2, 4.8 Hz, 1H). MS-ESI (m/z) calc'd for C₁₈H₁₆N₃O [M+H]⁺: 290.1. Found 290.2.

### Example 2: 8-((3-(Isoxazol-4-yl)-1H-indazol-5-yl)oxy)-5,6,7,8-tetrahydroquinoline-3-carbonitrile, enantiomer 1 and 2

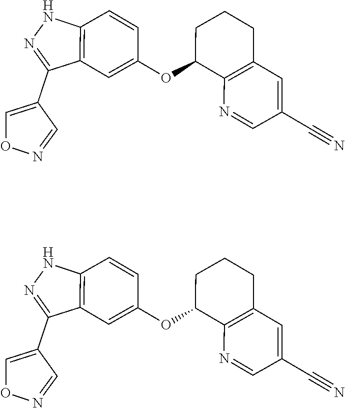 [SMILES: N#Cc1cnc2c(c1)CCC[C@@H]2Oc1ccc2[nH]nc(-c3cnoc3)c2c1 ; N#Cc1cnc2c(c1)CCC[C@H]2Oc1ccc2[nH]nc(-c3cnoc3)c2c1]

### Step 1: 8-((3-Isoxazol-4-yl)-1H-indazol-5-yl)oxy)-5,6,7,8-tetrahydroquinoline-3-carbonitrile

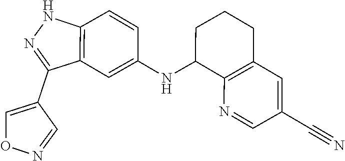 [SMILES: N#Cc1cnc2c(c1)CCCC2Nc1ccc2[nH]nc(-c3cnoc3)c2c1]

A microwave vial was charged with tert-butyl 5-((3-cyano-5,6,7,8-tetrahydroquinolin-8-yl)oxy)-3-iodo-1H-indazole-1-carboxylate (100.0 mg, 0.190 mmol), Pd(amphos)Cl₂ (13.34 mg, 0.020 mmol), isoxazole-4-boronic acid (21.2 mg, 0.190 mmol), 1,4-dioxane (1.779 mL) and water (0.445 mL). The vial was flushed with N₂ for 5 min, then KOAc (33.18 mg, 0.340 mmol) was added. The vial was sealed and irradiated at 100° C. for 30 min. The reaction mixture was concentrated to afford the title compound (26 mg, 38%) which was used without further purification.

### Step 2: 8-((3-(Isoxazol-4-yl)-1H-indazol-5-yl)oxy)-5,6,7,8-tetrahydroquinoline-3-carbonitrile, enantiomer 1 and 2

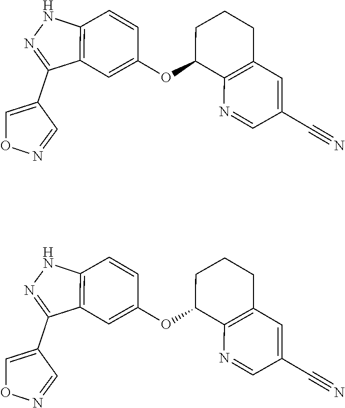 [SMILES: N#Cc1cnc2c(c1)CCC[C@@H]2Oc1ccc2[nH]nc(-c3cnoc3)c2c1 ; N#Cc1cnc2c(c1)CCC[C@H]2Oc1ccc2[nH]nc(-c3cnoc3)c2c1]

8-((3-Isoxazol-4-yl)-1H-indazol-5-yl)oxy)-5,6,7,8-tetrahydroquinoline-3-carbonitrile was subjected to chiral separation using Method B to afford 8-((3-(isoxazol-4-yl)-1H-indazol-5-yl)oxy)-5,6,7,8-tetrahydroquinoline-3-carbonitrile, enantiomer 1 (8.5 mg, 13%). ¹H NMR (400 MHz, DMSO-d₆) δ 13.19 (s, 1H), 9.75 (s, 1H), 9.18 (s, 1H), 8.86 (d, J=2.0 Hz, 1H), 8.21 (d, J=2.0 Hz, 1H), 7.67 (d, J=2.2 Hz, 1H), 7.51 (d, J=9.0 Hz, 1H), 7.16 (dd, J=9.0, 2.2 Hz, 1H), 5.70 (d, J=4.1 Hz, 1H), 3.04-2.75 (m, 2H), 2.31-2.22 (m, 1H), 2.08-1.87 (m, 2H), 1.82 (d, J=10.5 Hz, 1H). MS-ESI (m/z) calc'd for C₂₀H₁₆N₅O₂ [M+H]⁺: 358.1. Found 358.3. A second fraction was isolated to afford 8-((3-(isoxazol-4-yl)-1H-indazol-5-yl)oxy)-5,6,7,8-tetrahydroquinoline-3-carbonitrile, enantiomer 2 (7.8 mg, 12%). ¹H NMR (400 MHz, DMSO-d₆) δ 13.18 (s, 1H), 9.75 (s, 1H), 9.18 (s, 1H), 8.86 (d, J=2.1 Hz, 1H), 8.21 (d, J=2.0 Hz, 1H), 7.67 (d, J=2.2 Hz, 1H), 7.51 (d, J=9.0 Hz, 1H), 7.16 (dd, J=9.0, 2.2 Hz, 1H), 5.70 (d, J=4.0 Hz, 1H), 3.02-2.77 (m, 2H), 2.26 (d, J=12.5 Hz, 1H), 2.07-1.75 (m, 3H). ). MS-ESI (m/z) calc'd for C₂₀H₁₆N₅O₂ [M+H]⁺: 358.1. Found 358.3.

### Example 3: 1-((3-(Oxazol-5-yl)-1H-indazol-5-yl)oxy)-2,3-dihydro-1H-indene-5-carbonitrile, enantiomer 1 and 2

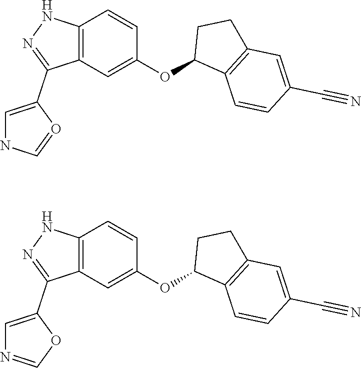

### Step 1: 1-((3-Oxazol-5-yl)-1H-indazol-5-yl)oxy)-2,3-dihydro-1H-indene-5-carbonitrile

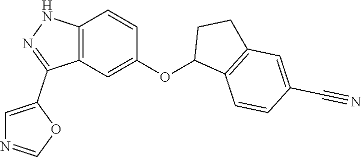 [SMILES: N#Cc1ccc2c(c1)CCC2Oc1ccc2[nH]nc(-c3cnco3)c2c1]

A microwave vial was charged with tert-butyl 5-[(5-cyano-2,3-dihydro-1H-inden-1-yl)oxy]-3-iodoindazole-1-carboxylate (100.0 mg, 0.200 mmol), Pd(amphos)Cl₂ (14.16 mg, 0.020 mmol), 5-(4,4,5,5-tetramethyl-1,3,2-dioxaborolan-2-yl)-1,3-oxazole (42.79 mg, 0.220 mmol), 1,4-dioxane (2.49 mL) and water (0.623 mL). The vial was flushed with N₂ for 5 min, then KOAc (35.24 mg, 0.360 mmol) was added. The vial was sealed and irradiated at 100° C. for 30 min. The reaction mixture was concentrated to afford the title compound (29 mg, 38%) which was used without further purification.

### Step 2: 1-((3-(Oxazol-5-yl)-1H-indazol-5-yl)oxy)-2,3-dihydro-1H-indene-5-carbonitrile, enantiomer 1 and 2

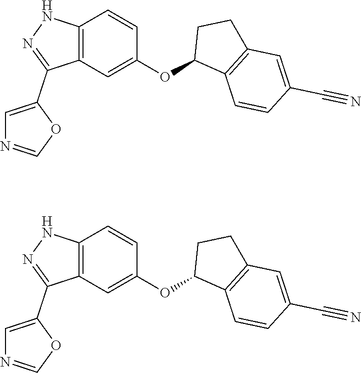 [SMILES: N#Cc1ccc2c(c1)CC[C@@H]2Oc1ccc2[nH]nc(-c3cnco3)c2c1 ; N#Cc1ccc2c(c1)CC[C@H]2Oc1ccc2[nH]nc(-c3cnco3)c2c1]

1-((3-(Oxazol-5-yl)-1H-indazol-5-yl)oxy)-2,3-dihydro-1H-indene-5-carbonitrile was subjected to chiral separation using Method C to afford 1-((3-(oxazol-5-yl)-1H-indazol-5-yl)oxy)-2,3-dihydro-1H-indene-5-carbonitrile, enantiomer 1 (7.1 mg, 10%). ¹H NMR (400 MHz, DMSO-d₆) δ 13.40 (s, 1H), 8.50 (s, 1H), 7.83 (d, J=2.1 Hz, 2H), 7.70 (dd, J=7.8, 1.5 Hz, 1H), 7.59 (dd, J=5.1, 2.8 Hz, 2H), 7.55 (d, J=9.0 Hz, 1H), 7.16 (dd, J=9.0, 2.3 Hz, 1H), 6.09 (dd, J=6.8, 4.8 Hz, 1H), 3.18-3.04 (m, 1H), 2.97 (dt, J=16.1, 7.2 Hz, 1H), 2.74-2.59 (m, 1H), 2.20-2.04 (m, 1H). MS-ESI (m/z) calc'd for C₂₀H₁₅N₄O₂ [M+H]⁺: 343.1. Found 343.2. A second fraction was isolated to afford 1-((3-(oxazol-5-yl)-1H-indazol-5-yl)oxy)-2,3-dihydro-1H-indene-5-carbonitrile, enantiomer 2 (8.3 mg, 12%). ¹H NMR (400 MHz, DMSO-d₆) δ 13.41 (s, 1H), 8.51 (s, 1H), 7.84 (d, J=2.3 Hz, 2H), 7.70 (dd, J=7.8, 1.5 Hz, 1H), 7.62-7.52 (m, 3H), 7.16 (dd, J=9.0, 2.3 Hz, 1H), 6.09 (dd, J=6.8, 4.8 Hz, 1H), 3.19-3.05 (m, 1H), 2.97 (dt, J=16.2, 7.3 Hz, 1H), 2.67 (dddd, J=13.5, 8.4, 6.8, 5.3 Hz, 1H), 2.19-2.05 (m, 1H). ). MS-ESI (m/z) calc'd for C₂₀H₁₅N₄O₂ [M+H]⁺: 343.1. Found 343.2.

### Example 4: 1-((3-(1-(Difluoromethyl)-1H-pyrazol-4-yl)-1H-indazol-5-yl)oxy)-2,3-dihydro-1H-indene-5-carbonitrile, enantiomer 1 and 2

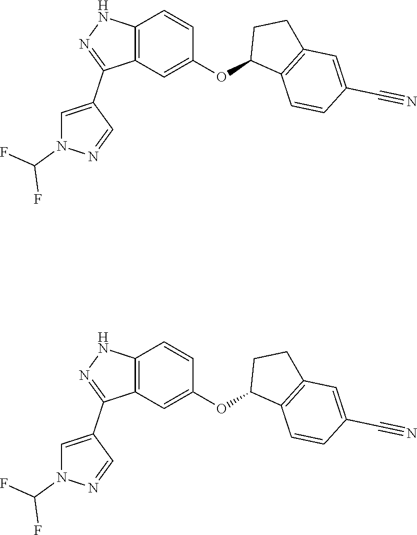 [SMILES: N#Cc1ccc2c(c1)CC[C@@H]2Oc1ccc2[nH]nc(-c3cnn(C(F)F)c3)c2c1 ; N#Cc1ccc2c(c1)CC[C@H]2Oc1ccc2[nH]nc(-c3cnn(C(F)F)c3)c2c1]

### Step 1: 1-(Difluoromethyl)-4-(4,4,5,5-tetramethyl-1,3,2-dioxaborolan-2-yl)-1H-pyrazole

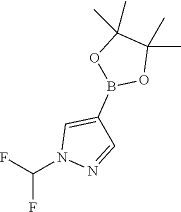 [SMILES: CC1(C)OB(c2cnn(C(F)F)c2)OC1(C)C]

4-(4,4,5,5-tetramethyl-1,3,2-dioxaborolan-2-yl)-1H-pyrazole (0.97 g, 5 mmol) and 1,4,7,10,13,16-hexaoxacyclooctadecane (0.26 g, 1 mmol) were dissolved in MeCN (25 mL). Sodium 2-chloro-2,2-difluoroacetate (0.91 g, 6 mmol) was added and the mixture was stirred at 80° C. for 6 hrs. The solvent was evaporated and the residue was purified by column chromatography on silica gel using a 0-40% EtOAc-cyclohexane gradient (10 CV) as eluent to afford the title compound (755 mg, 62%) as a colorless oil. ¹H NMR (400 MHz, DMSO-d₆) δ ppm 8.50-8.33 (m, 1H), 7.94-7.87 (m, 1H), 3.40-3.24 (m, 1H), 1.33-1.22 (m 12H). MS-ESI (m/z) calc'd for C₁₀H₁₆FB₂N₂O₂ [M+H]⁺: 245.1. Found 245.0.

### Step 2: 1-((3-(Oxazol-5-yl)-1H-indazol-5-yl)oxy)-2,3-dihydro-1H-indene-5-carbonitrile

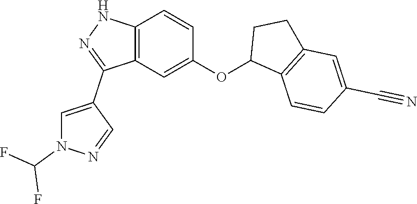 [SMILES: N#Cc1ccc2c(c1)CCC2Oc1ccc2[nH]nc(-c3cnn(C(F)F)c3)c2c1]

A microwave vial was charged with tert-butyl 5-[(5-cyano-2,3-dihydro-1H-inden-1-yl)oxy]-3-iodoindazole-1-carboxylate (70.0 mg, 0.140 mmol), Pd(amphos)Cl₂ (9.92 mg, 0.010 mmol), 1-(difluoromethyl)-4-(4,4,5,5-tetramethyl-1,3,2-dioxaborolan-2-yl)-1H-pyrazole (34.08 mg, 0.140 mmol), 1,4-dioxane (1.245 mL) and water (0.311 mL). The vial was flushed with N₂ for 5 min and then KOAc (24.67 mg, 0.250 mmol) was added. The vial was sealed and irradiated at 100° C. for 30 min. The reaction mixture was concentrated to afford the title compound (32 mg, 59%).

### Step 3: 1-((3-(1-(Difluoromethyl)-1H-pyrazol-4-yl)-1H-indazol-5-yl)oxy)-2,3-dihydro-1H-indene-5-carbonitrile, enantiomer 1 and 2

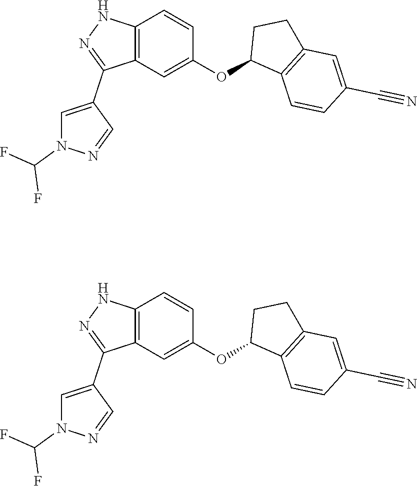 [SMILES: N#Cc1ccc2c(c1)CC[C@@H]2Oc1ccc2[nH]nc(-c3cnn(C(F)F)c3)c2c1 ; N#Cc1ccc2c(c1)CC[C@H]2Oc1ccc2[nH]nc(-c3cnn(C(F)F)c3)c2c1]

1-((3-(oxazol-5-yl)-1H-indazol-5-yl)oxy)-2,3-dihydro-1H-indene-5-carbonitrile was subjected to chiral separation using Method D to afford 1-((3-(1-(difluoromethyl)-1H-pyrazol-4-yl)-1H-indazol-5-yl)oxy)-2,3-dihydro-1H-indene-5-carbonitrile, enantiomer 1 (7 mg, 13%). ¹H NMR (400 MHz, DMSO-d₆) δ 13.07 (s, 1H), 8.90 (s, 1H), 8.37 (s, 1H), 7.87 (t, J=59.0 Hz, 1H), 7.84 (s, 1H), 7.70 (dd, J=7.8, 1.5 Hz, 1H), 7.64 (d, J=2.3 Hz, 1H), 7.59 (d, J=7.9 Hz, 1H), 7.51 (d, J=9.0 Hz, 1H), 7.13 (dd, J=9.0, 2.3 Hz, 1H), 6.14 (dd, J=6.8, 4.7 Hz, 1H), 3.17-3.07 (m, 1H), 3.02-2.91 (m, 1H), 2.72-2.59 (m, 1H), 2.19-2.07 (m, 1H). MS-ESI (m/z) calc'd for C₂₁H₁₆FN₅O [M+H]⁺: 392.1. Found 392.3. A second fraction was isolated to afford 1-((3-(1-(difluoromethyl)-1H-pyrazol-4-yl)-1H-indazol-5-yl)oxy)-2,3-dihydro-1H-indene-5-carbonitrile, enantiomer 2 (13 mg, 24%). ¹H NMR (400 MHz, DMSO-d₆) δ 13.08 (s, 1H), 8.90 (s, 1H), 8.37 (s, 1H), 8.02-7.82 (m, 2H), 7.73-7.63 (m, 2H), 7.55 (dd, J=32.4, 8.4 Hz, 2H), 7.13 (dd, J=9.0, 2.3 Hz, 1H), 6.14 (dd, J=6.8, 4.8 Hz, 1H), 3.19-2.88 (m, 2H), 2.72-2.60 (m, 1H), 2.19-2.04 (m, 1H). MS-ESI (m/z) calc'd for C₂₁H₁₆F₂N₅O [M+H]⁺: 392.1. Found 392.3.

### Example 5: 1-((3-(Isoxazol-4-yl)-1H-indazol-5-yl)oxy)-2,3-dihydro-1H-indene-5-carbonitrile, enantiomer 1 and 2

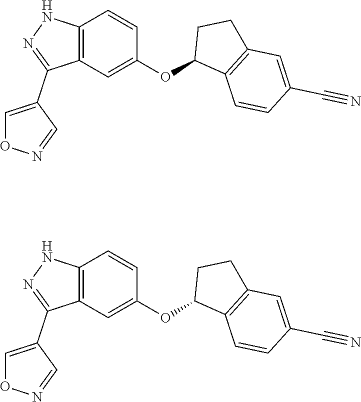 [SMILES: N#Cc1ccc2c(c1)CC[C@@H]2Oc1ccc2[nH]nc(-c3cnoc3)c2c1 ; N#Cc1ccc2c(c1)CC[C@H]2Oc1ccc2[nH]nc(-c3cnoc3)c2c1]

### Step 1: 1-((3-(Isoxazol-4-yl)-1H-indazol-5-yl)oxy)-2,3-dihydro-1H-indene-5-carbonitrile

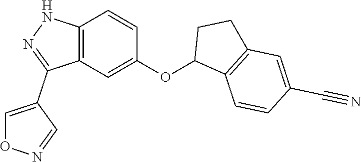 [SMILES: N#Cc1ccc2c(c1)CCC2Oc1ccc2[nH]nc(-c3cnoc3)c2c1]

A microwave vial was charged with tert-butyl 5-[(5-cyano-2,3-dihydro-1H-inden-1-yl)oxy]-3-iodoindazole-1-carboxylate (70.0 mg, 0.140 mmol), Pd(amphos)Cl₂ (9.92 mg, 0.010 mmol), isoxazole-4-boronic acid (15.76 mg, 0.140 mmol), 1,4-dioxane (1.245 mL) and water (0.311 mL). The vial was flushed with N₂ for 5 min, then KOAc (24.67 mg, 0.250 mmol) was added. The vial was sealed and irradiated at 100° C. for 30 min. The reaction mixture was evaporated to dryness to afford the title compound (32 mg, 71%).

### Step 2: 1-((3-(Isoxazol-4-yl)-1H-indazol-5-yl)oxy)-2,3-dihydro-1H-indene-5-carbonitrile, enantiomer 1 and 2

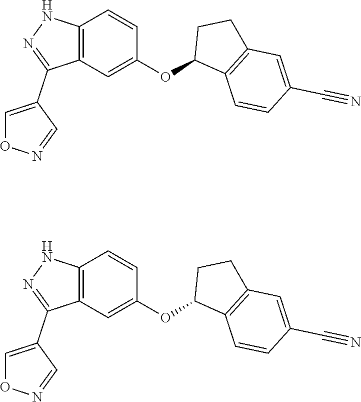 [SMILES: N#Cc1ccc2c(c1)CC[C@@H]2Oc1ccc2[nH]nc(-c3cnoc3)c2c1 ; N#Cc1ccc2c(c1)CC[C@H]2Oc1ccc2[nH]nc(-c3cnoc3)c2c1]

1-((3-(Oxazol-5-yl)-1H-indazol-5-yl)oxy)-2,3-dihydro-1H-indene-5-carbonitrile was subjected to chiral separation using Method E to afford 1-((3-(isoxazol-4-yl)-1H-indazol-5-yl)oxy)-2,3-dihydro-1H-indene-5-carbonitrile, enantiomer 1 (7.1 mg, 15%). ¹H NMR (400 MHz, DMSO-d₆) δ 13.21 (s, 1H), 9.76 (s, 1H), 9.19 (s, 1H), 7.84 (s, 1H), 7.70 (dd, J=7.8, 1.5 Hz, 1H), 7.64 (d, J=2.2 Hz, 1H), 7.56 (dd, J=25.1, 8.4 Hz, 2H), 7.14 (dd, J=9.0, 2.3 Hz, 1H), 6.16-6.10 (m, 1H), 3.17-2.91 (m, 2H), 2.75-2.65 (m, 1H), 2.11 (ddt, J=13.5, 8.6, 5.5 Hz, 1H). MS-ESI (m/z) calc'd for C₂₀H₁₅N₄O₂ [M+H]⁺: 343.3. Found 343.3. A second fraction was isolated to afford 1-((3-(isoxazol-4-yl)-1H-indazol-5-yl)oxy)-2,3-dihydro-1H-indene-5-carbonitrile, enantiomer 2 (3.5 mg, 7%). ¹H NMR (400 MHz, DMSO-d₆) δ 13.21 (s, 1H), 9.76 (s, 1H), 9.19 (s, 1H), 7.84 (s, 1H), 7.70 (dd, J=7.8, 1.5 Hz, 1H), 7.64 (d, J=2.3 Hz, 1H), 7.56 (dd, J=25.0, 8.4 Hz, 2H), 7.14 (dd, J=9.0, 2.3 Hz, 1H), 6.13 (dd, J=6.8, 4.8 Hz, 1H), 3.18-2.91 (m, 2H), 2.69 (dddd, J=13.5, 9.0, 6.9, 5.2 Hz, 1H), 2.18-2.00 (m, 1H). MS-ESI (m/z) calc'd for C₂₀H₁₅N₄O₂ [M+H]⁺: 343.3. Found 343.3.

### Example 6: 5-((3-(Isoxazol-4-yl)-1H-indazol-5-yl)amino)-5,6,7,8-tetrahydronaphthalene-2-carbonitrile, enantiomer 1 and 2

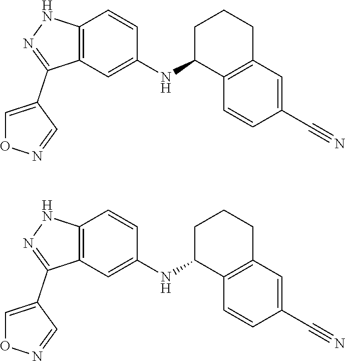 [SMILES: N#Cc1ccc2c(c1)CCC[C@@H]2Nc1ccc2[nH]nc(-c3cnoc3)c2c1 ; N#Cc1ccc2c(c1)CCC[C@H]2Nc1ccc2[nH]nc(-c3cnoc3)c2c1]

### Step 1: 5-((3-(Isoxazol-4-yl)-1H-indazol-5-yl)amino)-5,6,7,8-tetrahydronaphthalene-2-carbonitrile

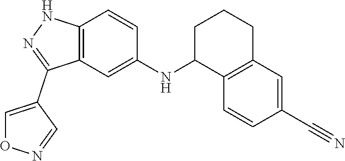 [SMILES: N#Cc1ccc2c(c1)CCCC2Nc1ccc2[nH]nc(-c3cnoc3)c2c1]

To a stirred solution of 3-(isoxazol-4-yl)-1H-indazol-5-amine (120.45 mg, 0.600 mmol) and 6-cyano-1-tetralone (100.0 mg, 0.580 mmol) in toluene (1.87 mL) was added 4-methylbenzenesulfonic acid hydrate (11.11 mg, 0.060 mmol) and the mixture was stirred at 110° C. overnight. The reaction was left to reach r.t. and then warmed to 40° C. NaBH(OAc)₃ (371.39 mg, 1.75 mmol) was added portionwise over 2 hrs. The solvent was evaporated and the residue was taken up in water and extracted with EtOAc. The organic layer was washed with brine, and evaporated to afford the title compound (188 mg, 88%).

### Step 2: 5-((3-(Isoxazol-4-yl)-1H-indazol-5-yl)amino)-5,6,7,8-tetrahydronaphthalene-2-carbonitrile, enantiomer 1 and 2

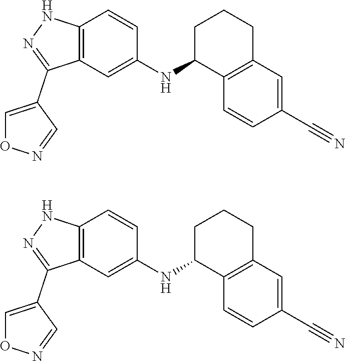 [SMILES: N#Cc1ccc2c(c1)CCC[C@@H]2Nc1ccc2[nH]nc(-c3cnoc3)c2c1 ; N#Cc1ccc2c(c1)CCC[C@H]2Nc1ccc2[nH]nc(-c3cnoc3)c2c1]

5-((3-(Isoxazol-4-yl)-1H-indazol-5-yl)amino)-5,6,7,8-tetrahydronaphthalene-2-carbonitrile was subjected to chiral separation using Method F to afford 5-((3-(isoxazol-4-yl)-1H-indazol-5-yl)amino)-5,6,7,8-tetrahydronaphthalene-2-carbonitrile, enantiomer 1 (4.14 mg, 2%). ¹H NMR (400 MHz, methanol-d₄) δ 9.25 (s, 1H), 8.92 (s, 1H), 7.61 (d, J=8.0 Hz, 1H), 7.51 (d, J=1.6 Hz, 1H), 7.46 (dd, J=8.0, 1.8 Hz, 1H), 7.40-7.35 (m, 1H), 7.07-7.00 (m, 2H), 4.82 (d, J=4.3 Hz, 1H), 2.87 (qd, J=17.1, 8.6 Hz, 2H), 2.19-2.04 (m, 1H), 2.04-1.81 (m, 3H). MS-ESI (m/z) calc'd for C₂₁H₁₈N₅O [M+H]⁺: 356.1. Found 356.3. A second fraction was isolated to afford 5-((3-(isoxazol-4-yl)-1H-indazol-5-yl)amino)-5,6,7,8-tetrahydronaphthalene-2-carbonitrile, enantiomer 2 (3.6 mg, 2%). ¹H NMR (400 MHz, methanol-d₄) δ 9.25 (s, 1H), 8.92 (s, 1H), 7.61 (d, J=8.0 Hz, 1H), 7.51 (d, J=1.7 Hz, 1H), 7.46 (dd, J=8.0, 1.8 Hz, 1H), 7.41-7.36 (m, 1H), 7.06-7.00 (m, 2H), 4.83-4.80 (m, 1H), 2.88 (qd, J=17.0, 8.7 Hz, 2H), 2.16-2.05 (m, 1H), 2.04-1.83 (m, 3H). MS-ESI (m/z) calc'd for C₂₁H₁₈N₅O [M+H]⁺: 356.1. Found 356.3.

### Example 7: 8-((3-(Isoxazol-4-yl)-1H-indazol-5-yl)amino)-5,6,7,8-tetrahydroquinoline-3-carbonitrile, enantiomer 1 and 2

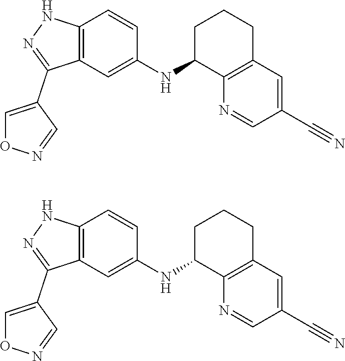 [SMILES: N#Cc1cnc2c(c1)CCC[C@@H]2Nc1ccc2[nH]nc(-c3cnoc3)c2c1 ; N#Cc1cnc2c(c1)CCC[C@H]2Nc1ccc2[nH]nc(-c3cnoc3)c2c1]

### Step 1: 8-((3-(Isoxazol-4-yl)-1H-indazol-5-yl)amino)-5,6,7,8-tetrahydroquinoline-3-carbonitrile

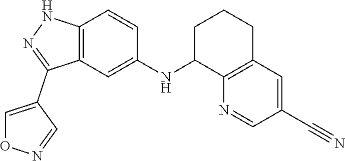 [SMILES: N#Cc1cnc2c(c1)CCCC2Nc1ccc2[nH]nc(-c3cnoc3)c2c1]

To a stirred solution of 3-(isoxazol-4-yl)-1H-indazol-5-amine (60.0 mg, 0.300 mmol) and 8-oxo-5,6,7,8-tetrahydroquinoline-3-carbonitrile (129.01 mg, 0.750 mmol) in 1,4-dioxane (0.865 mL) was added acetic acid (0.06 g, 0.980 mmol) and the mixture was stirred at 100° C. for 4 hrs. NaBH(OAc)₃ (0.17 g, 0.900 mmol) was added portionwise over 2 hrs. The solvent was evaporated and the residue was taken up in water and extracted with EtOAc. The organic layer was washed with brine and concentrated. This material was purified by prep HPLC using Method G to afford the title compound (2.9 mg, 3%).

### Step 2: 8-((3-(Isoxazol-4-yl)-1H-indazol-5-yl)amino)-5,6,7,8-tetrahydroquinoline-3-carbonitrile, enantiomer 1 and 2

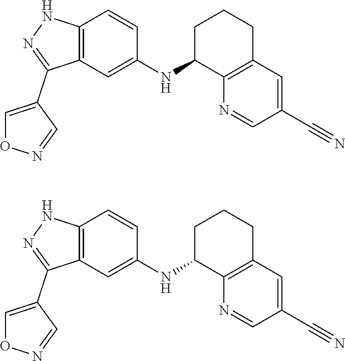 [SMILES: N#Cc1cnc2c(c1)CCC[C@@H]2Nc1ccc2[nH]nc(-c3cnoc3)c2c1 ; N#Cc1cnc2c(c1)CCC[C@H]2Nc1ccc2[nH]nc(-c3cnoc3)c2c1]

8-((3-(Isoxazol-4-yl)-1H-indazol-5-yl)amino)-5,6,7,8-tetrahydroquinoline-3-carbonitrile was subjected to chiral separation using Method H to afford 8-((3-(isoxazol-4-yl)-1H-indazol-5-yl)amino)-5,6,7,8-tetrahydroquinoline-3-carbonitrile, enantiomer 1 (1.2 mg, 1%). ¹H NMR (400 MHz, methanol-d₄) δ 8.48 (s, 1H), 8.14 (s, 1H), 7.91-7.86 (m, 1H), 7.18 (dd, J=2.0, 1.0 Hz, 1H), 6.58 (dd, J=9.0, 0.7 Hz, 1H), 6.32 (d, J=2.0 Hz, 1H), 6.26 (dd, J=8.9, 2.1 Hz, 1H), 3.99 (t, J=5.4 Hz, 1H), 2.23-2.05 (m, 2H), 1.45-1.08 (m, 4H). MS-ESI (m/z) calc'd for C₂₀H₁₇N₆O [M+H]⁺: 357.1. Found 357.2. A second fraction was isolated to afford 8-((3-(isoxazol-4-yl)-1H-indazol-5-yl)amino)-5,6,7,8-tetrahydroquinoline-3-carbonitrile, enantiomer 2 (1.6 mg, 2%). ¹H NMR (400 MHz, methanol-d₄) δ 8.48 (s, 1H), 8.14 (s, 1H), 7.89 (dd, J=2.0, 1.0 Hz, 1H), 7.18 (dt, J=2.0, 1.0 Hz, 1H), 6.58 (dd, J=9.0, 0.7 Hz, 1H), 6.32 (d, J=2.0 Hz, 1H), 6.26 (dd, J=8.9, 2.1 Hz, 1H), 3.99 (t, J=5.3 Hz, 1H), 2.13 (dtd, J=24.4, 17.5, 6.6 Hz, 2H), 1.50-1.10 (m, 4H). MS-ESI (m/z) calc'd for C₂₀H₁₇N₆O [M+H]⁺: 357.1. Found 357.2.

### Example 8: 1-((3-(Isoxazol-4-yl)-1H-indazol-5-yl)amino)-2,3-dihydro-1H-indene-5-carbonitrile, enantiomer 1 and 2

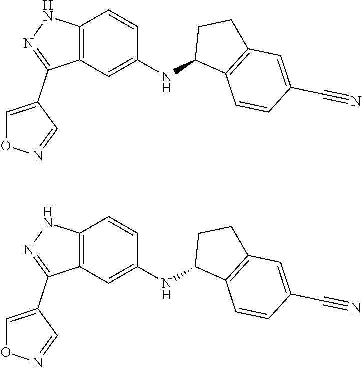 [SMILES: N#Cc1ccc2c(c1)CC[C@@H]2Nc1ccc2[nH]nc(-c3cnoc3)c2c1 ; N#Cc1ccc2c(c1)CC[C@H]2Nc1ccc2[nH]nc(-c3cnoc3)c2c1]

### Step 1: 1-((3-(Isoxazol-4-yl)-1H-indazol-5-yl)amino)-2,3-dihydro-1H-indene-5-carbonitrile

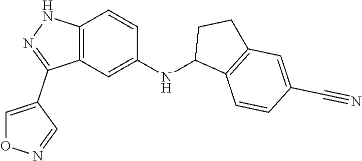 [SMILES: N#Cc1ccc2c(c1)CCC2Nc1ccc2[nH]nc(-c3cnoc3)c2c1]

To a stirred solution of 3-(isoxazol-4-yl)-1H-indazol-5-amine (150.0 mg, 0.740 mmol) and 1-oxo-2,3-dihydroindene-5-carbonitrile (291.46 mg, 1.85 mmol) in 1,4-dioxane (2.141 mL) was added acetic acid (0.15 g, 2.42 mmol) and the mixture was stirred at 100° C. for 4 hrs. Sodium triacetoxyborohydride (0.43 g, 2.23 mmol) was added portionwise over 2 hrs. The solvent was evaporated and the residue was taken up in water and extracted with EtOAc. The organic layer was washed with brine and evaporated to obtain material that was purified by silica gel chromatography using a 0-60% gradient of EtOAc in cyclohexane (12 CV) as eluent to afford the title compound (23 mg, 9%) as a yellow oil.

### Step 2: 1-((3-(Isoxazol-4-yl)-1H-indazol-5-yl)amino)-2,3-dihydro-1H-indene-5-carbonitrile, enantiomer 1 and 2

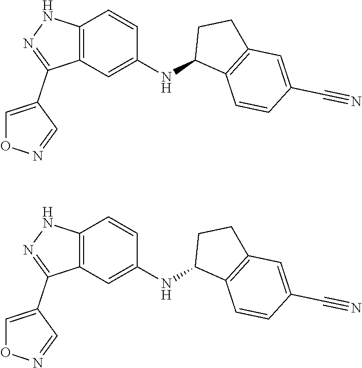 [SMILES: N#Cc1ccc2c(c1)CC[C@@H]2Nc1ccc2[nH]nc(-c3cnoc3)c2c1 ; N#Cc1ccc2c(c1)CC[C@H]2Nc1ccc2[nH]nc(-c3cnoc3)c2c1]

1-((3-(Isoxazol-4-yl)-1H-indazol-5-yl)amino)-2,3-dihydro-1H-indene-5-carbonitrile was subjected to chiral separation using Method I to afford 1-((3-(Isoxazol-4-yl)-1H-indazol-5-yl)amino)-2,3-dihydro-1H-indene-5-carbonitrile, enantiomer 1 (5.7 mg, 2%). ¹H NMR (400 MHz, DMSO-d₆) δ 12.91 (s, 1H), 9.61 (s, 1H), 9.11 (s, 1H), 7.76 (s, 1H), 7.66-7.59 (m, 1H), 7.46 (d, J=7.8 Hz, 1H), 7.36 (d, J=8.9 Hz, 1H), 7.13 (d, J=2.1 Hz, 1H), 6.97 (dd, J=8.9, 2.0 Hz, 1H), 5.91 (d, J=9.2 Hz, 1H), 5.33 (q, J=8.0 Hz, 1H), 3.09-2.85 (m, 2H), 2.71-2.58 (m, 1H), 1.91-1.76 (m, 1H). MS-ESI (m/z) calc'd for C₂₀H₁₆N₅O [M+H]⁺: 342.1. Found 342.3. A second fraction was isolated to afford 1-((3-(isoxazol-4-yl)-1H-indazol-5-yl)amino)-2,3-dihydro-1H-indene-5-carbonitrile, enantiomer 2 (5.2 mg, 2%). ¹H NMR (400 MHz, DMSO-d₆) δ 12.91 (s, 1H), 9.61 (s, 1H), 9.11 (s, 1H), 7.76 (s, 1H), 7.62 (dd, J=8.2, 1.5 Hz, 1H), 7.41 (dd, J=41.7, 8.4 Hz, 2H), 7.13 (d, J=2.0 Hz, 1H), 6.97 (dd, J=8.9, 2.0 Hz, 1H), 5.91 (d, J=9.2 Hz, 1H), 5.34 (q, J=7.8 Hz, 1H), 3.10-2.82 (m, 2H), 2.71-2.59 (m, 1H), 1.83 (dq, J=12.3, 8.6 Hz, 1H). MS-ESI (m/z) calc'd for C₂₀H₁₆N₅O [M+H]⁺: 342.1. Found 342.2.

### Example 9: 1-((3-(Oxazol-5-yl)-1H-indazol-5-yl)amino)-2,3-dihydro-1H-indene-5-carbonitrile, enantiomer 1 and 2

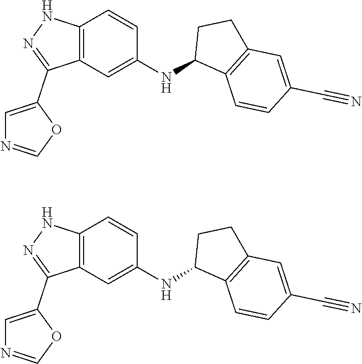 [SMILES: N#Cc1ccc2c(c1)CC[C@@H]2Nc1ccc2[nH]nc(-c3cnco3)c2c1 ; N#Cc1ccc2c(c1)CC[C@H]2Nc1ccc2[nH]nc(-c3cnco3)c2c1]

### Step 1: 1-((3-(Oxazol-5-yl)-1H-indazol-5-yl)amino)-2,3-dihydro-1H-indene-5-carbonitrile

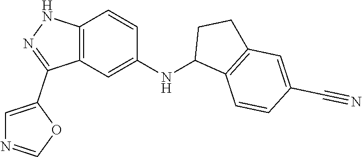 [SMILES: N#Cc1ccc2c(c1)CCC2Nc1ccc2[nH]nc(-c3cnco3)c2c1]

To a stirred solution of 3-(1,3-oxazol-5-yl)-1H-indazol-5-amine (167.0 mg, 0.750 mmol) and 1-oxo-2,3-dihydroindene-5-carbonitrile (294.99 mg, 1.88 mmol) in 1,4-dioxane (2.167 mL) was added acetic acid (0.14 mL, 2.45 mmol) and the mixture was stirred at 100° C. for 4 hrs. Sodium triacetoxyborohydride (0.43 g, 2.25 mmol) was added portionwise over 2 hrs. The solvent was evaporated and the residue was taken up in water and extracted with EtOAc. The organic layer was washed with brine and evaporated to obtain a dark oil which was purified by prep HPLC Method J to afford the title compound (28 mg, 11%) as a green solid.

### Step 2: 1-((3-(Oxazol-5-yl)-1H-indazol-5-yl)amino)-2,3-dihydro-1H-indene-5-carbonitrile, enantiomer 1 and 2

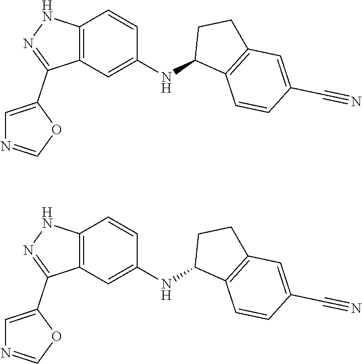 [SMILES: N#Cc1ccc2c(c1)CC[C@@H]2Nc1ccc2[nH]nc(-c3cnco3)c2c1 ; N#Cc1ccc2c(c1)CC[C@H]2Nc1ccc2[nH]nc(-c3cnco3)c2c1]

1-((3-(Oxazol-5-yl)-1H-indazol-5-yl)amino)-2,3-dihydro-1H-indene-5-carbonitrile was subjected to chiral separation using Method K to afford 1-[[3-(1,3-oxazol-5-yl)-1H-indazol-5-yl]amino]-2,3-dihydro-1H-indene-5-carbonitrile, enantiomer 1 (8.3 mg, 3%). ¹H NMR (400 MHz, DMSO-d₆) δ 13.12 (s, 1H), 8.45 (s, 1H), 7.76 (s, 1H), 7.67-7.61 (m, 2H), 7.42 (dd, J=28.1, 8.4 Hz, 2H), 7.11 (d, J=2.1 Hz, 1H), 7.01 (dd, J=9.0, 2.1 Hz, 1H), 6.02 (d, J=8.8 Hz, 1H), 5.24 (q, J=8.0 Hz, 1H), 3.10-2.84 (m, 2H), 2.70-2.57 (m, 1H), 1.87 (dd, J=12.5, 8.3 Hz, 1H). MS-ESI (m/z) calc'd for C₂₀H₁₆N₅O [M+H]⁺: 342.1. Found 342.3. A second fraction was isolated to afford 1-((3-(oxazol-5-yl)-1H-indazol-5-yl)amino)-2,3-dihydro-1H-indene-5-carbonitrile, enantiomer 2 (8.1 mg, 3%). ¹H NMR (400 MHz, DMSO-d₆) δ 13.12 (s, 1H), 8.45 (s, 1H), 7.76 (s, 1H), 7.69-7.58 (m, 2H), 7.42 (dd, J=28.5, 8.4 Hz, 2H), 7.11 (d, J=2.0 Hz, 1H), 7.01 (dd, J=9.0, 2.1 Hz, 1H), 6.02 (d, J=8.8 Hz, 1H), 5.24 (q, J=8.0 Hz, 1H), 3.08-2.85 (m, 2H), 2.70-2.58 (m, 1H), 1.87 (dq, J=12.4, 8.6 Hz, 1H). MS-ESI (m/z) calc'd for C₂₀H₁₆N₅O [M+H]⁺: 342.1. Found 342.3.

### Example 10: 1-((3-(Furan-3-yl)-1H-indazol-5-yl)amino)-2,3-dihydro-1H-indene-5-carbonitrile

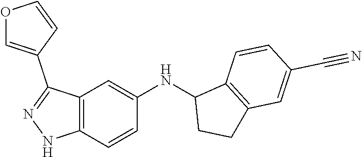 [SMILES: N#Cc1ccc2c(c1)CCC2Nc1ccc2[nH]nc(-c3ccoc3)c2c1]

To a stirred solution of 3-(furan-3-yl)-1H-indazol-5-amine (190.12 mg, 0.950 mmol) in toluene 2 mL was added titanium(IV) chloride 1.0 M in DCM (0.64 mL, 0.640 mmol) for 30 min at r.t. under nitrogen atmosphere. The resulting mixture was stirred at 90° C. for 30 min followed by addition of 1-oxo-2,3-dihydroindene-5-carbonitrile (100.0 mg, 0.640 mmol). The resulting mixture was stirred for 10 min at 90° C., poured into water and extracted with EtOAc. The organic layer was separated, dried over Na₂SO₄ and then concentrated. The resulting solid was dissolved in 2.0 mL of methanol and NaBH₃CN (59.97 mg, 0.950 mmol) was added followed by 1 drop of acetic acid. The mixture was refluxed for 3 hrs under an argon atmosphere, cooled to r.t. and concentrated. The residue was diluted with 3 mL of water and extracted with EtOAc. The combined organic extracts were dried over Na₂SO₄ and concentrated. The residue was purified by preparative HPLC Method L to afford the title compound (1.6 mg, 1%). ¹H NMR (400 MHz, methanol-d₄) δ 8.09 (dd, J=1.54, 0.88 Hz, 1H), 7.65 (s, 1H), 7.63 (t, J=1.76 Hz, 1H), 7.52-7.55 (m, 2H), 7.39 (d, J=9.02 Hz, 1H), 7.11 (s, 1H), 7.08 (dd, J=8.80, 2.20 Hz, 1H), 6.98 (dd, J=1.87, 0.77 Hz, 1H), 5.23 (t, J=7.37 Hz, 1H), 2.96-3.17 (m, 2H), 2.65-2.75 (m, 1H), 1.96-2.08 (m, 1H). MS-ESI (m/z) calc'd for C₂₁H₁₇N₄O [M+H]⁺: 341.1. Found 341.2.

### Example 11: 1-((3-(Furan-3-yl)-1H-indazol-5-yl)amino)-2,3-dihydro-1H-indene-5-carbonitrile, enantiomer 1 and 2

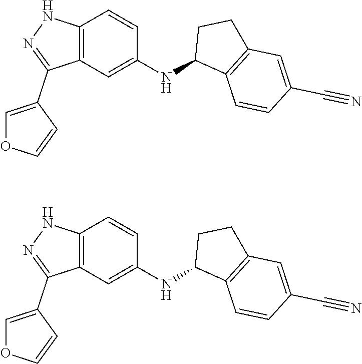 [SMILES: N#Cc1ccc2c(c1)CC[C@@H]2Nc1ccc2[nH]nc(-c3ccoc3)c2c1 ; N#Cc1ccc2c(c1)CC[C@H]2Nc1ccc2[nH]nc(-c3ccoc3)c2c1]

### Step 1: 1-((3-(Furan-3-yl)-1H-indazol-5-yl)amino)-2,3-dihydro-1H-indene-5-carbonitrile

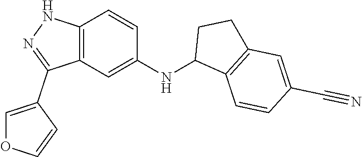 [SMILES: N#Cc1ccc2c(c1)CCC2Nc1ccc2[nH]nc(-c3ccoc3)c2c1]

To a stirred solution of 3-(furan-3-yl)-1H-indazol-5-amine (0.5 g, 2.5 mmol) and 1-oxo-2,3-dihydroindene-5-carbonitrile (0.98 g, 6.25 mmol) in 1,4-dioxane (7.5 mL) was added acetic acid (0.47 mL, 8.16 mmol) and the mixture was stirred at 100° C. for 4 hrs. Sodium triacetoxyborohydride (1.44 g, 7.5 mmol) was added portionwise over 2 hrs. The solvent was evaporated, the residue was taken up in water and extracted with EtOAc (3×), the combined organic layers were washed with brine and evaporated to obtain a residue which was purified by column chromatography on silica gel using a 0-70% gradient of EtOAc in cyclohexane (15 CV) as eluent to obtain a solid which was dissolved in a minimum of Et₂O and precipitated with cyclohexane (4V with respect to Et₂O), then left standing for 15 hrs. The solid was filtered and dried to afford the title compound (300 mg, 35%) as a beige solid. ¹H NMR (400 MHz, DMSO-d₆) δ 12.68 (s, 1H), 8.31 (t, J=1.2 Hz, 1H), 7.76 (dd, J=3.6, 1.8 Hz, 2H), 7.62 (dd, J=7.8, 1.6 Hz, 1H), 7.46 (d, J=7.8 Hz, 1H), 7.32 (d, J=8.8 Hz, 1H), 7.07 (d, J=2.1 Hz, 1H), 7.00-6.87 (m, 2H), 5.83 (d, J=9.1 Hz, 1H), 5.29 (q, J=8.0 Hz, 1H), 3.02 (ddd, J=16.2, 8.8, 3.2 Hz, 1H), 2.91 (dt, J=16.3, 8.3 Hz, 1H), 2.61 (ddt, J=11.0, 7.4, 3.2 Hz, 1H), 1.85 (dq, J=12.4, 8.6 Hz, 1H). MS-ESI (m/z) calc'd for C₂₁H₁₇N₄O [M+H]⁺: 341.1. Found 341.1.

### Step 2: 1-((3-(Furan-3-yl)-1H-indazol-5-yl)amino)-2,3-dihydro-1H-indene-5-carbonitrile, enantiomer 1 and 2

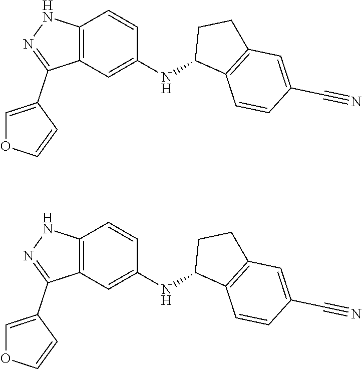 [SMILES: N#Cc1ccc2c(c1)CC[C@H]2Nc1ccc2[nH]nc(-c3ccoc3)c2c1 ; N#Cc1ccc2c(c1)CC[C@H]2Nc1ccc2[nH]nc(-c3ccoc3)c2c1]

1-((3-(Furan-3-yl)-1H-indazol-5-yl)amino)-2,3-dihydro-1H-indene-5-carbonitrile was subjected to chiral separation using Method M to afford 1-((3-(furan-3-yl)-1H-indazol-5-yl)amino)-2,3-dihydro-1H-indene-5-carbonitrile, enantiomer 1 (110 mg, 79%). ¹H NMR (400 MHz, DMSO-d₆) δ 12.68 (s, 1H), 8.31 (t, J=1.1 Hz, 1H), 7.81-7.72 (m, 2H), 7.62 (dd, J=7.9, 1.5 Hz, 1H), 7.46 (d, J=7.8 Hz, 1H), 7.32 (d, J=8.8 Hz, 1H), 7.10-7.03 (m, 1H), 7.00-6.90 (m, 2H), 5.83 (d, J=9.1 Hz, 1H), 5.29 (q, J=8.0 Hz, 1H), 3.02 (ddd, J=16.3, 8.7, 3.3 Hz, 1H), 2.91 (dt, J=16.4, 8.4 Hz, 1H), 2.63 (tdd, J=11.0, 7.4, 3.5 Hz, 1H), 1.85 (dq, J=12.3, 8.6 Hz, 1H). MS-ESI (m/z) calc'd for C₂₁H₁₇N₄O [M+H]⁺: 341.1. Found 341.2. A second fraction was isolated to afford 1-((3-(furan-3-yl)-1H-indazol-5-yl)amino)-2,3-dihydro-1H-indene-5-carbonitrile, enantiomer 2 (114 mg, 81%). ¹H NMR (400 MHz, DMSO-d₆) δ 12.68 (s, 1H), 8.31 (t, J=1.2 Hz, 1H), 7.83-7.73 (m, 2H), 7.62 (d, J=7.6 Hz, 1H), 7.46 (d, J=7.8 Hz, 1H), 7.32 (d, J=8.9 Hz, 1H), 7.07 (s, 1H), 7.01-6.89 (m, 2H), 5.83 (d, J=9.1 Hz, 1H), 5.29 (q, J=8.0 Hz, 1H), 3.02 (ddd, J=16.1, 8.9, 3.4 Hz, 1H), 2.91 (dt, J=16.2, 8.4 Hz, 1H), 2.62 (ddt, J=11.0, 7.4, 3.8 Hz, 1H), 1.85 (dq, J=12.4, 8.7 Hz, 1H). Chiral SFC: 10.7. MS-ESI (m/z) calc'd for C₂₁H₁₇N₄O [M+H]⁺: 341.1. Found 341.2.

### Example 12: 1-((3-(1-(Difluoromethyl)-1H-pyrazol-4-yl)-1H-indazol-5-yl)amino)-2,3-dihydro-1H-indene-5-carbonitrile, enantiomer 1 and 2

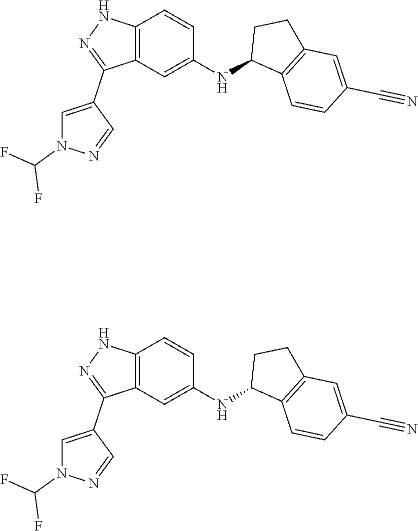 [SMILES: N#Cc1ccc2c(c1)CC[C@@H]2Nc1ccc2[nH]nc(-c3cnn(C(F)F)c3)c2c1 ; N#Cc1ccc2c(c1)CC[C@H]2Nc1ccc2[nH]nc(-c3cnn(C(F)F)c3)c2c1]

### Step 1: 3-(1-(Difluoromethyl)-1H-pyrazol-4-yl)-1H-indazol-5-amine

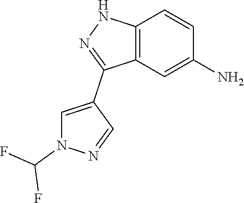 [SMILES: Nc1ccc2[nH]nc(-c3cnn(C(F)F)c3)c2c1]

3-Iodo-1H-indazole-5-amine (1.3 g, 5 mmol) was dissolved in THF (28.37 mL). A solution of K₃PO₄ (3.18 g, 15 mmol) and 1-(difluoromethyl)-4-(4,4,5,5-tetramethyl-1,3,2-dioxaborolan-2-yl)pyrazole (1.46 g, 6 mmol) in water (10.64 mL) was added and the mixture was degassed with N₂ for 15 minutes. SPhos-Pd-G2 (0.36 g, 0.500 mmol) was added and the mixture was stirred at 100° C. under N₂ for 1 hr. Water was added and the organic solvent was evaporated. The solid formed was filtered, washed with water, and dried. The filtrate was extracted with EtOAc (3×) and the combined organic layers were evaporated and added to the solid to obtain a residue which triturated with DCM to afford the title compound (880 mg, 70%) as a grey solid. ¹H NMR (400 MHz, DMSO-d₆) δ 12.70 (s, 1H), 8.61 (s, 1H), 8.22 (s, 1H), 7.88 (t, J=59.1 Hz, 1H), 7.28 (d, J=8.8 Hz, 1H), 7.03 (d, J=2.0 Hz, 1H), 6.84 (dd, J=8.8, 2.0 Hz, 1H), 4.82 (s, 2H). MS-ESI (m/z) calc'd for C₁₁H₁₀F₂N₅ [M+H]⁺: 250.1. Found 250.3.

### Step 2: 1-((3-(1-(Difluoromethyl)-1H-pyrazol-4-yl)-1H-indazol-5-yl)amino)-2,3-dihydro-1H-indene-5-carbonitrile

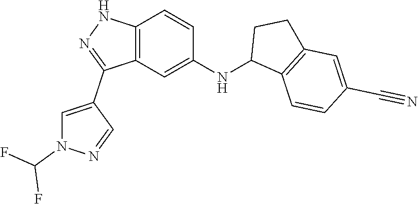 [SMILES: N#Cc1ccc2c(c1)CCC2Nc1ccc2[nH]nc(-c3cnn(C(F)F)c3)c2c1]

To a stirred solution of 3-[1-(difluoromethyl)pyrazol-4-yl]-1H-indazol-5-amine (249.22 mg, 1 mmol) and 1-oxo-2,3-dihydroindene-5-carbonitrile (392.92 mg, 2.5 mmol) in 1,4-dioxane (3 mL) was added acetic acid (0.19 mL, 3.26 mmol) and the mixture was stirred at 100° C. for 4 hrs. Sodium triacetoxyborohydride (0.58 g, 3 mmol) was added portionwise over 2 hrs. The solvent was evaporated and the residue was taken up in water and extracted with EtOAc. The organic layer was washed with brine and concentrated. The material was purified by prep HPLC Method N to afford the title compound (175 mg, 45%) as a green solid. ¹H NMR (400 MHz, DMSO-d₆) δ 12.79 (s, 1H), 8.71 (d, J=0.7 Hz, 1H), 8.28 (s, 1H), 7.85 (t, J=59.1 Hz, 1H), 7.75 (d, J=1.5 Hz, 1H), 7.62 (dd, J=7.8, 1.5 Hz, 1H), 7.46 (d, J=7.8 Hz, 1H), 7.34 (d, J=8.9 Hz, 1H), 7.13 (d, J=2.1 Hz, 1H), 6.97 (dd, J=8.9, 2.1 Hz, 1H), 5.86 (d, J=9.2 Hz, 1H), 5.32 (q, J=8.0 Hz, 1H), 3.04 (ddd, J=16.4, 8.8, 3.3 Hz, 1H), 2.91 (dt, J=16.4, 8.4 Hz, 1H), 2.62 (dtd, J=12.4, 7.7, 3.4 Hz, 1H), 1.87 (dq, J=12.5, 8.6 Hz, 1H). MS-ESI (m/z) calc'd for C₂₁H₁₇F₂N₆ [M+H]⁺: 391.1. Found 391.2.

### Step 3: 1-((3-(1-(Difluoromethyl)-1H-pyrazol-4-yl)-1H-indazol-5-yl)amino)-2,3-dihydro-1H-indene-5-carbonitrile, enantiomer 1 and 2

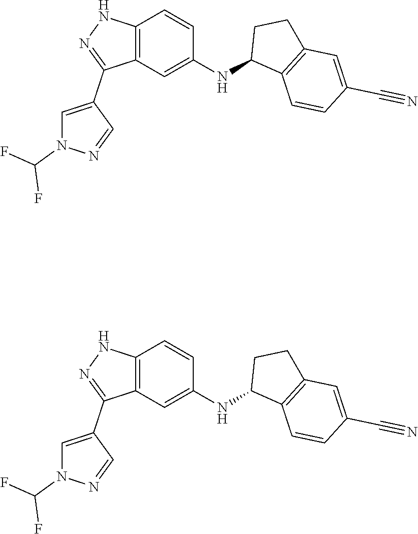 [SMILES: N#Cc1ccc2c(c1)CC[C@@H]2Nc1ccc2[nH]nc(-c3cnn(C(F)F)c3)c2c1 ; N#Cc1ccc2c(c1)CC[C@H]2Nc1ccc2[nH]nc(-c3cnn(C(F)F)c3)c2c1]

1-((3-(1-(Difluoromethyl)-1H-pyrazol-4-yl)-1H-indazol-5-yl)amino)-2,3-dihydro-1H-indene-5-carbonitrile was subjected to chiral separation using Method O to afford 1-((3-(1-(difluoromethyl)-1H-pyrazol-4-yl)-1H-indazol-5-yl)amino)-2,3-dihydro-1H-indene-5-carbonitrile, enantiomer 1 (55 mg, 63%). ¹H NMR (400 MHz, DMSO-d₆) δ 12.78 (s, 1H), 8.71 (d, J=0.7 Hz, 1H), 8.28 (d, J=0.7 Hz, 1H), 7.85 (t, J=59.1 Hz, 1H), 7.75 (d, J=1.5 Hz, 1H), 7.62 (dd, J=7.8, 1.5 Hz, 1H), 7.46 (d, J=7.8 Hz, 1H), 7.34 (d, J=8.9 Hz, 1H), 7.13 (d, J=2.1 Hz, 1H), 6.97 (dd, J=8.9, 2.1 Hz, 1H), 5.86 (d, J=9.2 Hz, 1H), 5.32 (q, J=8.0 Hz, 1H), 3.04 (ddd, J=16.4, 8.7, 3.3 Hz, 1H), 2.91 (dt, J=16.3, 8.4 Hz, 1H), 2.69-2.56 (m, 1H), 1.87 (dq, J=12.5, 8.6 Hz, 1H). MS-ESI (m/z) calc'd for C₂₁H₁₇F₂N₆ [M+H]⁺: 391.1. Found 391.3. A second fraction was isolated to afford 1-((3-(1-(difluoromethyl)-1H-pyrazol-4-yl)-1H-indazol-5-yl)amino)-2,3-dihydro-1H-indene-5-carbonitrile carbonitrile, enantiomer 2 (56 mg, 64%). ¹H NMR (400 MHz, DMSO-d₆) δ 12.78 (s, 1H), 8.71 (d, J=0.7 Hz, 1H), 8.28 (d, J=0.6 Hz, 1H), 7.85 (t, J=59.1 Hz, 1H), 7.75 (d, J=1.4 Hz, 1H), 7.62 (dd, J=7.8, 1.5 Hz, 1H), 7.46 (d, J=7.8 Hz, 1H), 7.34 (d, J=8.9 Hz, 1H), 7.13 (d, J=2.1 Hz, 1H), 6.97 (dd, J=8.9, 2.0 Hz, 1H), 5.86 (d, J=9.2 Hz, 1H), 5.32 (q, J=8.0 Hz, 1H), 3.04 (ddd, J=16.3, 8.7, 3.3 Hz, 1H), 2.91 (dt, J=16.3, 8.4 Hz, 1H), 2.69-2.56 (m, 1H), 1.87 (dq, J=12.5, 8.6 Hz, 1H). MS-ESI (m/z) calc'd for C₂₁H₁₇F₂N₆ [M+H]⁺: 391.1. Found 391.3.

### Example 13: 1-((3-Iodo-1H-indazol-5-yl)oxy)-2,3-dihydro-1H-indene-5-carbonitrile

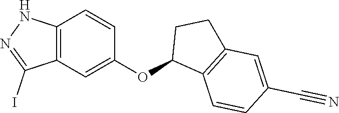 [SMILES: N#Cc1ccc2c(c1)CC[C@@H]2Oc1ccc2[nH]nc(I)c2c1]

### Step 1: 1-Hydroxy-2,3-dihydro-1H-indene-5-carbonitrile, enantiomer 1

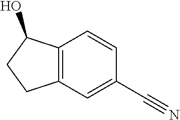 [SMILES: N#Cc1ccc2c(c1)CC[C@H]2O]

Potassium hexacyanoferrate(II), 0.1 N standardized solution (11.73 mL, 1.17 mmol), (R)-5-bromo-2,3-dihydro-1H-inden-1-ol (500.0 mg, 2.35 mmol) and KOAc (460.6 mg, 4.69 mmol) were dissolved in a mixture of 1,4-dioxane (40 mL)/water (5.7 mL) under N₂. XPHOS (111.87 mg, 0.230 mmol) and XPHOS-Pd-G3 (198.63 mg, 0.230 mmol) were added and the mixture was stirred at 105° C. for 18 hrs. The reaction mixture was partitioned between water and EtOAc and the phases were separated. The aqueous layer was extracted with EtOAc and the combined organic phases were washed with brine, dried over Na₂SO₄ and concentrated. The material was purified by column chromatography on silica gel using a 0-50% gradient of EtOAc in DCM to afford the title compound (100 mg, 27%) as an off-white solid. ¹H NMR (400 MHz, DMSO-d₆) δ 7.73-7.62 (m, 2H), 7.49 (d, J=7.70 Hz, 1H), 5.50 (d, J=5.94 Hz, 1H), 5.08 (q, J=6.60 Hz, 1H), 3.01-2.87 (m, 1H), 2.75 (dt, J=16.23, 8.06 Hz, 1H), 2.44-2.30 (m, 1H), 1.87-1.71 (m, 1H). MS-ESI (m/z) calc'd for C₁₀H₁₀NO [M+H]⁺: 160.1. Found 160.0.

### Step 2: tert-Butyl 5-((5-cyano-2,3-dihydro-1H-inden-1-yl)oxy)-3-iodo-1H-indazole-1-carboxylate, enantiomer 1

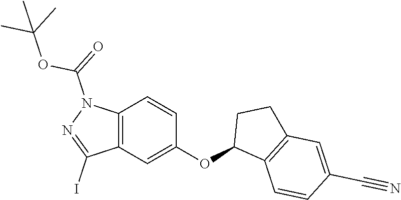 [SMILES: CC(C)(C)OC(=O)n1nc(I)c2cc(O[C@H]3CCc4cc(C#N)ccc43)ccc21]

1-Hydroxy-2,3-dihydro-1H-indene-5-carbonitrile, enantiomer 1 (100.0 mg, 0.630 mmol) was dissolved in THF (4.286 mL) and stirred under N₂. tert-Butyl 5-hydroxy-3-iodo-1H-indazole-1-carboxylate (511.5 mg, 1.42 mmol) and triphenylphosphine (409.77 mg, 1.56 mmol) were added followed by dropwise addition of diethyl azodicarboxylate (0.22 mL, 1.42 mmol) at 0° C. The reaction was stirred at r.t. for 1 hr. The mixture was then quenched with water and partitioned between water and EtOAc. The organic layer was separated, washed with brine, dried over Na₂SO₄, filtered and concentrated. The material was purified by column chromatography on silica gel using a 0-50% gradient of EtOAc in cyclohexane (10 CV) to afford the title compound (220 mg, 70%) as a white solid. ¹H NMR (400 MHz, DMSO-d₆) δ 7.99 (d, J=9.1 Hz, 1H), 7.84 (s, 1H), 7.75-7.66 (m, 1H), 7.59 (d, J=7.9 Hz, 1H), 7.39 (dd, J=9.1, 2.4 Hz, 1H), 7.12 (d, J=2.4 Hz, 1H), 6.14-6.07 (m, 1H), 3.11 (ddd, J=14.3, 8.6, 5.3 Hz, 1H), 2.98 (dt, J=16.0, 7.1 Hz, 1H), 2.66 (td, J=13.7, 6.8 Hz, 1H), 2.16-2.05 (m, 1H), 1.64 (s, 9H). MS-ESI (m/z) calc'd for C₂₂H₂₁IN₃O₃ [M+H]⁺: 502.1. Found 502.0.

### Step 3: 1-((3-Iodo-1H-indazol-5-yl)oxy)-2,3-dihydro-1H-indene-5-carbonitrile, enantiomer 1

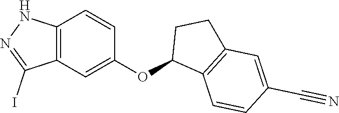 [SMILES: N#Cc1ccc2c(c1)CC[C@@H]2Oc1ccc2[nH]nc(I)c2c1]

To a solution of tert-butyl 5-((5-cyano-2,3-dihydro-1H-inden-1-yl)oxy)-3-iodo-1H-indazole-1-carboxylate, enantiomer 1 (220.0 mg, 0.440 mmol) in 3 mL of DCM was added trifluoroacetic acid (0.500 mL) at 0° C. The reaction mixture was stirred at r.t. for 3 hrs and then concentrated under reduced pressure. The material was purified by reversed phase column chromatography using a 2-100% gradient of MeCN in H₂O (0.1% formic acid, 10 CV). This material was then further purified by column chromatography on silica gel using a 0-50% gradient of EtOAc in cyclohexane (8 CV) to obtain the title compound (52 mg, 29%). ¹H NMR (400 MHz, DMSO-d₆) δ 13.41 (s, 1H), 7.84 (d, J=1.5 Hz, 1H), 7.71 (dd, J=7.9, 1.5 Hz, 1H), 7.58 (d, J=7.8 Hz, 1H), 7.50 (d, J=8.9 Hz, 1H), 7.14 (dd, J=9.0, 2.3 Hz, 1H), 6.99 (d, J=2.3 Hz, 1H), 6.01 (dd, J=6.8, 4.8 Hz, 1H), 3.19-3.05 (m, 1H), 2.98 (dt, J=16.0, 7.2 Hz, 1H), 2.74-2.58 (m, 1H), 2.12 (ddt, J=13.6, 8.7, 5.7 Hz, 1H). MS-ESI (m/z) calc'd for C₁₇H₁₃IN₃O [M+H]⁺: 402.0. Found 402.0.

### Example 14: 7-Methyl-1-((3-(oxazol-5-yl)-1H-indazol-5-yl)oxy)-2,3-dihydro-1H-indene-5-carbonitrile, enantiomer 1 and 2

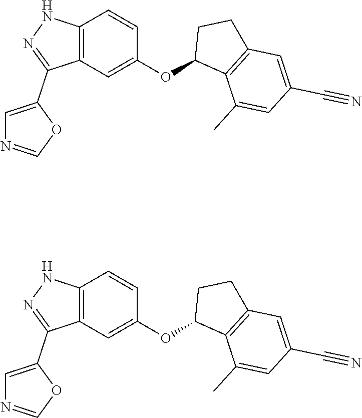 [SMILES: Cc1cc(C#N)cc2c1[C@@H](Oc1ccc3[nH]nc(-c4cnco4)c3c1)CC2 ; Cc1cc(C#N)cc2c1[C@H](Oc1ccc3[nH]nc(-c4cnco4)c3c1)CC2]

### Step 1: 5-Bromo-7-methyl-2,3-dihydro-1H-inden-1-ol

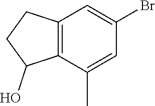

To a solution of 5-bromo-7-methyl-2,3-dihydro-1H-inden-1-one (250.0 mg, 1.11 mmol) in MeOH (15 mL) was added sodium borohydride (84.04 mg, 2.22 mmol). The resulting clear solution was allowed to stir for 2 hrs at 25° C. The reaction was quenched with water and extracted with DCM. The combined organic layers were dried on sodium sulfate, filtered and concentrated to afford the title compound (244 mg, 96%) as a white solid. ¹H NMR (400 MHz, DMSO-d₆) δ 7.23 (s, 1H), 7.16 (d, J=0.96 Hz, OH), 5.06 (td, J=6.44, 3.02 Hz, 1H), 4.99 (d, J=6.38 Hz, 1H), 2.99 (dt, J=15.84, 7.74 Hz, 1H), 2.32 (s, 3H), 2.60-2.80 (m, 1H), 2.23 (ddt, J=13.72, 8.85, 7.17 Hz, 1H), 1.85 (dddd, J=13.41, 8.37, 4.27, 3.08 Hz, 1H). MS-ESI (m/z) calc'd for C₁₀H₁₂BrO [M+H]⁺: 227.0. Found 225.7, 227.0.

### Step 2: 1-Hydroxy-7-methyl-2,3-dihydro-1H-indene-5-carbonitrile

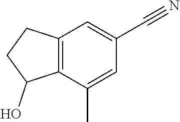

In a sealed microwave vial, 5-bromo-7-methyl-2,3-dihydro-1H-inden-1-ol (80.0 mg, 0.350 mmol) and KOAc (69.14 mg, 0.700 mmol) were dissolved in a mixture of 1,4-dioxane (3 mL)/water (0.500 mL). Potassium hexacyanoferrate (II), 0.1 N standardized solution (3.52 mL, 0.350 mmol) was added and the mixture was degassed with N₂ for 15 minutes. Then XPHOS (134.35 mg, 0.280 mmol) and [2-(2-aminophenyl)phenyl]palladium(1+); dicyclohexyl-[2-[2,4,6-tri(propan-2-yl)phenyl]phenyl]phosphane methanesulfonate; hydrofluoride (0.28 mL, 0.280 mmol) were added and the mixture was stirred at 100° C. for 6 hrs. The reaction mixture was partitioned between water and EtOAc and the phases were separated. The aqueous layer was extracted with EtOAc and the combined organic phases were washed with water, dried over Na₂SO₄ and concentrated. The material was purified by column chromatography on silica gel using a 0-50% EtOAc/DCM gradient eluent. The first eluted fractions were taken up in cyclohexane and filtered to afford the title compound (25 mg, 41%) as an off-white solid. ¹H NMR (400 MHz, CDCl₃) δ 7.37 (s, 1H), 7.31 (s, 1H), 5.35 (td, J=6.73, 3.06 Hz, 1H), 2.91-2.71 (m, 1H), 3.25-3.07 (m, 1H), 2.51-2.41 (m, 1H), 2.46 (s, 3H), 2.08 (dddd, J=14.05, 8.44, 4.16, 3.06 Hz, 1H). MS-ESI (m/z) calc'd for C₁₁H₁₂NO [M+H]⁺: 174.1. Found 174.2.

### Step 3: tert-Butyl 5-((5-cyano-7-methyl-2,3-dihydro-1H-inden-1-yl)oxy)-3-iodo-1H-indazole-1-carboxylate

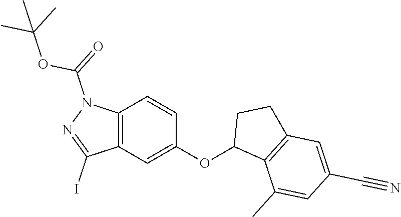 [SMILES: Cc1cc(C#N)cc2c1C(Oc1ccc3c(c1)c(I)nn3C(=O)OC(C)(C)C)CC2]

1-Hydroxy-7-methyl-2,3-dihydro-1H-indene-5-carbonitrile (25.0 mg, 0.140 mmol) was dissolved in THF (1 mL) and stirred under N₂. tert-Butyl 5-hydroxy-3-iodo-1H-indazole-1-carboxylate (511.5 mg, 1.42 mmol) and triphenylphosphine (409.77 mg, 1.56 mmol) were added followed by dropwise addition of diethyl azodicarboxylate (0.22 mL, 1.42 mmol) at 0° C. After stirring for 3 hrs, the resulting solution was partitioned between water and EtOAc. The organic layer was separated, washed with brine, dried over Na₂SO₄, filtered and concentrated. The material was purified by column chromatography on silica gel using a 30-70% EtOAc/cyclohexane gradient eluent to afford the title compound (28 mg, 37%) as a white solid. ¹H NMR (400 MHz, CDCl₃) δ 8.06 (d, J=9.26 Hz, 1H), 7.41 (s, 1H), 7.48 (s, 1H), 7.23 (dd, J=9.11, 2.45 Hz, 1H), 6.96 (d, J=2.50 Hz, 1H), 5.89 (dd, J=6.68, 2.40 Hz, 1H), 3.26 (dt, J=16.50, 8.07 Hz, 1H), 3.12-2.91 (m, 1H), 2.76-2.51 (m, 1H), 2.40 (s, 3H), 1.74 (s, 8H). MS-ESI (m/z) calc'd for C₁₁H₁₂NO [[M−t-Bu+H]⁺: 416.0. Found [M−t-Bu+H]⁺416.0.

### Step 4: 7-Methyl-1-((3-(oxazol-5-yl)-1H-indazol-5-yl)oxy)-2,3-dihydro-1H-indene-5-carbonitrile

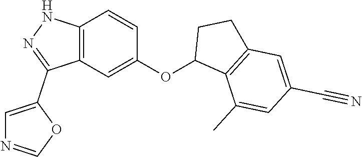 [SMILES: Cc1cc(C#N)cc2c1C(Oc1ccc3[nH]nc(-c4cnco4)c3c1)CC2]

A microwave vial was charged with tert-butyl 5-((5-cyano-7-methyl-2,3-dihydro-1H-inden-1-yl)oxy)-3-iodo-1H-indazole-1-carboxylate (27.0 mg, 0,050 mmol), 5-(4,4,5,5-tetramethyl-1,3,2-dioxaborolan-2-yl)-1,3-oxazole (11.24 ng, 0.060 mmol) and KOAc (9.26 mg, 0.090 mmol), 1,4-dioxane (0.500 mL) and water (0.124 mL). The vial was flushed with N₂ for 15 min, then Pd(amphos)Cl₂ (3.72 mg, 0.010 mmol) was added. The vial was sealed and irradiated at 100° C. for 30 min. The material was partitioned between water and EtOAc, the aqueous layer was extracted with EtOAc and the combined organic layers were washed with water, dried with Na₂SO₄, filtered and concentration. The material was purified by column chromatography on silica gel using a 20-80% gradient of EtOAc in cyclohexane to afford the title compound (14 mg, 75%) as an off-white solid.

### Step 5: 7-Methyl-1-((3-(oxazol-5-yl)-1H-indazol-5-yl)oxy)-2,3-dihydro-1H-indene-5-carbonitrile, enantiomer 1 and 2

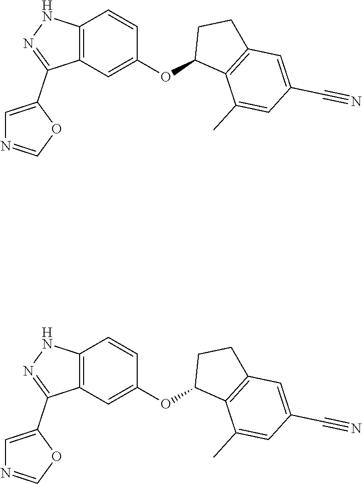 [SMILES: Cc1cc(C#N)cc2c1[C@@H](Oc1ccc3[nH]nc(-c4cnco4)c3c1)CC2 ; Cc1cc(C#N)cc2c1[C@H](Oc1ccc3[nH]nc(-c4cnco4)c3c1)CC2]

7-Methyl-1-((3-(oxazol-5-yl)-1H-indazol-5-yl)oxy)-2,3-dihydro-1H-indene-5-carbonitrile was subjected to chiral separation using Method Q to afford 7-methyl-1-((3-(oxazol-5-yl)-1H-indazol-5-yl)oxy)-2,3-dihydro-1H-indene-5-carbonitrile, enantiomer 1 (2.9 mg, 15%). ¹H NMR (400 MHz, DMSO-d₆) δ 13.41 (s, 1H), 8.51 (s, 1H), 7.83 (s, 1H), 7.66 (s, 1H), 7.57 (s, 2H), 7.53 (d, J=2.3 Hz, 1H), 7.14 (dd, J=9.0, 2.3 Hz, 1H), 6.09 (dd, J=6.7, 2.4 Hz, 1H), 3.13 (dt, J=16.2, 7.8 Hz, 1H), 2.97 (ddd, J=16.8, 9.0, 3.7 Hz, 1H), 2.59 (ddd, J=14.2, 7.1, 2.0 Hz, 1H), 2.35 (s, 3H), 2.18 (ddt, J=14.3, 8.6, 3.3 Hz, 1H). MS-ESI (m/z) calc'd for C₂₁H₁₇N₄O₂ [M+H]⁺: 357.1. Found 357.1. A second fraction was isolated to afford 7-methyl-1-((3-(oxazol-5-yl)-1H-indazol-5-yl)oxy)-2,3-dihydro-1H-indene-5-carbonitrile, enantiomer 2 (2.5 mg, 13%). ¹H NMR (400 MHz, DMSO-d₆) δ 13.40 (br. s., 1H) 8.51 (s, 1H) 7.82 (s, 1H) 7.66 (s, 1H) 7.50-7.60 (m, 3H) 7.13 (dd, J=8.80, 1.98 Hz, 1H) 6.09 (d, J=4.62 Hz, 1H) 3.13 (dt, J=16.01, 7.95 Hz, 1H) 2.97 (ddd, J=16.78, 8.97, 3.74 Hz, 1H) 2.55-2.64 (m, 1H) 2.34 (s, 3H) 2.11-2.24. MS-ESI (m/z) calc'd for C₂₁H₁₇N₄O₂ [M+H]⁺: 357.1. Found 357.1.

### Example 15: 5-((3-Iodo-1H-indazol-5-yl)amino)-5,6,7,8-tetrahydronaphthalene-2-carbonitrile, enantiomer 1 and 2

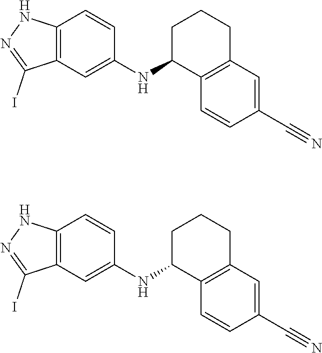 [SMILES: N#Cc1ccc2c(c1)CCC[C@@H]2Nc1ccc2[nH]nc(I)c2c1 ; N#Cc1ccc2c(c1)CCC[C@H]2Nc1ccc2[nH]nc(I)c2c1]

### Step 1: 5-((3-Iodo-1H-indazol-5-yl)amino)-5,6,7,8-tetrahydronaphthalene-2-carbonitrile

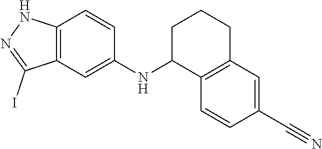 [SMILES: N#Cc1ccc2c(c1)CCCC2Nc1ccc2[nH]nc(I)c2c1]

To a stirred solution of 3-iodo-1H-indazol-5-amine (605.26 mg, 2.34 mmol) and 6-cyano-1-tetralone (200.0 mg, 1.17 mmol) in 1,4-dioxane (4.325 mL) was added 4-methylbenzenesulfonic acid hydrate (22.22 mg, 0.120 mmol) and the mixture was stirred at 105° C. for 4 hrs. The reaction was brought to r.t. and then heated to 40° C. NaBH(OAc)₃ (673.81 mg, 3.5 mmol) was added portionwise over 3 hrs and the mixture was stirred for 18 hrs at 40° C. The solvent was evaporated and the residue was taken up in water and extracted with EtOAc. The organic layer was washed with brine and concentrated. The material was purified by reversed phase column chromatography using a 2-100% gradient of MeCN in H₂O (0.1% formic acid, 8 CV) to afford the title compound (197 mg, 41%) as a white solid.

### Step 2: 5-((3-Iodo-1H-indazol-5-yl)amino)-5,6,7,8-tetrahydronaphthalene-2-carbonitrile, enantiomer 1 and 2

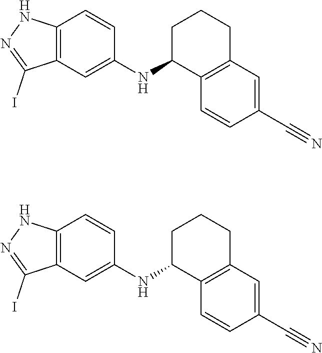 [SMILES: N#Cc1ccc2c(c1)CCC[C@@H]2Nc1ccc2[nH]nc(I)c2c1 ; N#Cc1ccc2c(c1)CCC[C@H]2Nc1ccc2[nH]nc(I)c2c1]

5-((3-Iodo-1H-indazol-5-yl)amino)-5,6,7,8-tetrahydronaphthalene-2-carbonitrile (20 mg) was subjected to chiral separation using Method R to afford 5-((3-iodo-1H-indazol-5-yl)amino)-5,6,7,8-tetrahydronaphthalene-2-carbonitrile, enantiomer 1 (6.6 mg). ¹H NMR (400 MHz, DMSO-d₆) δ 13.08 (s, 1H), 7.63 (d, J=1.7 Hz, 1H), 7.57 (dd, J=8.1, 1.8 Hz, 1H), 7.50 (d, J=8.0 Hz, 1H), 7.31 (d, J=8.9 Hz, 1H), 7.00 (dd, J=9.0, 2.1 Hz, 1H), 6.42 (d, J=2.0 Hz, 1H), 5.95 (d, J=8.9 Hz, 1H), 4.68 (d, J=8.0 Hz, 1H), 2.80 (dd, J=16.2, 9.1 Hz, 2H), 2.03-1.73 (m, 4H). MS-ESI (m/z) calc'd for C₁₈H₁₅IN₄ [M+H]⁺: 415.0. Found 415.0. A second fraction was isolated to afford 5-((3-iodo-1H-indazol-5-yl)amino)-5,6,7,8-tetrahydronaphthalene-2-carbonitrile, enantiomer 2 (5.9 mg). ¹H NMR (400 MHz, DMSO-d₆) δ 13.08 (s, 1H), 7.63 (d, J=1.7 Hz, 1H), 7.57 (dd, J=8.1, 1.8 Hz, 1H), 7.50 (d, J=8.0 Hz, 1H), 7.31 (d, J=8.9 Hz, 1H), 7.00 (dd, J=9.0, 2.1 Hz, 1H), 6.42 (d, J=2.0 Hz, 1H), 5.95 (d, J=8.9 Hz, 1H), 4.68 (d, J=8.0 Hz, 1H), 2.80 (dd, J=16.2, 9.1 Hz, 2H), 2.03-1.73 (m, 4H). MS-ESI (m/z) calc'd for C₁₈H₁₅IN₄ [M+H]⁺: 415.0. Found 415.1.

### Example 16: 8-((3-(Oxazol-5-yl)-1H-indazol-5-yl)oxy)-5,6,7,8-tetrahydroquinoline-3-carbonitrile, enantiomer 1 and 2

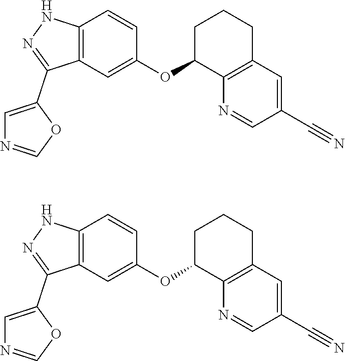 [SMILES: N#Cc1cnc2c(c1)CCC[C@@H]2Oc1ccc2[nH]nc(-c3cnco3)c2c1 ; N#Cc1cnc2c(c1)CCC[C@H]2Oc1ccc2[nH]nc(-c3cnco3)c2c1]

### Step 1: 8-Hydroxy-5,6,7,8-tetrahydroquinoline-3-carbonitrile

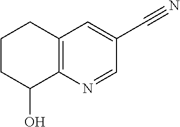 [SMILES: N#Cc1cnc2c(c1)CCCC2O]

A flask was charged with 8-oxo-5,6,7,8-tetrahydroquinoline-3-carbonitrile (100.57 mg, 0.580 mmol) and MeOH (10 mL). NaBH₄ (44.19 mg, 1.17 mmol) was then added to the stirred mixture and the resulting clear, colorless solution was allowed to stir for 30 min. Water was added and the aqueous layer was extracted with DCM. The combined organic layers were dried over Na₂SO₄, filtered, and concentrated under vacuum. The material was purified by column chromatography on silica gel using a 0-50% EtOAc/cyclohexane gradient eluent to afford the title compound (88 mg, 86%). MS-ESI (m/z) calc'd for C₁₀H₁₁N₂O [M+H]⁺: 175.1. Found 175.1.

### Step 2: tert-Butyl 5-((3-cyano-5,6,7,8-tetrahydroquinolin-8-yl)oxy)-3-iodo-1H-indazole-1-carboxylate

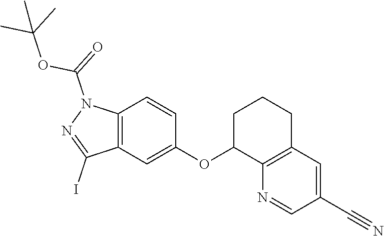 [SMILES: CC(C)(C)OC(=O)n1nc(I)c2cc(OC3CCCc4cc(C#N)cnc43)ccc21]

A solution of 2,8-hydroxy-5,6,7,8-tetrahydroquinoline-3-carbonitrile (85.0 mg, 0.490 mmol) in THF (0.600 mL) was added to a flask under an N₂ atmosphere. tert-Butyl 5-hydroxy-3-iodo-1H-indazole-1-carboxylate (175.73 mg, 0.490 mmol) and triphenylphosphine (140.78 mg, 0.540 mmol) were added followed by dropwise addition of diethyl azodicarboxylate (84.98 mg, 0.488 mL, 0.490 mmol). After stirring for 1 hr, the resulting solution was diluted with water and EtOAc and extracted. The organic layers were combined, washed with brine, dried over Na₂SO₄, filtered and concentrated. The material was purified by column chromatography on silica gel using a 0-20% EtOAc/cyclohexane gradient eluent to afford the title compound (211 mg, 84%). MS-ESI (m/z) calc'd for C₁₀H₁₁N₂O [M+H]⁺: 517.1. Found 517.1.

### Step 3: 8-((3-(Oxazol-5-yl)-1H-indazol-5-yl)oxy)-5,6,7,8-tetrahydroquinoline-3-carbonitrile

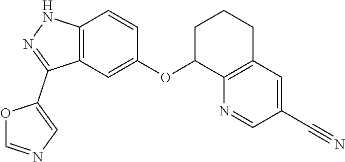 [SMILES: N#Cc1cnc2c(c1)CCCC2Oc1ccc2[nH]nc(-c3cnco3)c2c1]

A microwave vial was charged with tert-butyl 5-((3-cyano-5,6,7,8-tetrahydroquinolin-8-yl)oxy)-3-iodo-1H-indazole-1-carboxylate (109.0 mg, 0.210 mmol), Pd(amphos)Cl₂ (14.99 mg, 0.020 mmol), 5-(4,4,5,5-tetramethyl-1,3,2-dioxaborolan-2-yl)-1,3-oxazole (45.29 mg, 0.230 mmol), 1,4-dioxane (4 mL) and water (1 mL). The vial was flushed with N for 15 min followed by addition of KOAc (37.29 mg, 0.380 mmol). The vial was sealed and irradiated at 100° C. for 30 min. The material was partitioned between water and EtOAc and extracted.

The combined organic layers were washed with brine, dried over Na₂SO₄, filtered and concentrated. The material was purified by column chromatography on silica gel using a 30-20% EtOAc/cyclohexane gradient eluent to afford the title compound (50 mg, 66%) as an off-white solid.

### Step 4: 8-((3-(Oxazol-5-yl)-1H-indazol-5-yl)oxy)-5,6,7,8-tetrahydroquinoline-3-carbonitrile, enantiomer 1 and 2

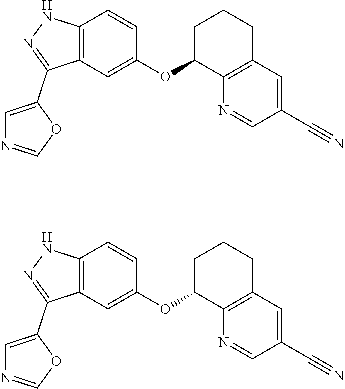 [SMILES: N#Cc1cnc2c(c1)CCC[C@@H]2Oc1ccc2[nH]nc(-c3cnco3)c2c1 ; N#Cc1cnc2c(c1)CCC[C@H]2Oc1ccc2[nH]nc(-c3cnco3)c2c1]

8-((3-(Oxazol-5-yl)-1H-indazol-5-yl)oxy)-5,6,7,8-tetrahydroquinoline-3-carbonitrile was subjected to chiral separation using Method S to afford 8-((3-(oxazol-5-yl)-1H-indazol-5-yl)oxy)-5,6,7,8-tetrahydroquinoline-3-carbonitrile, enantiomer 1 (6.6 mg, 1.3%). ¹H NMR (400 MHz, DMSO-d₆) δ 13.39 (s, 1H), 8.86 (d, J=2.03 Hz, 1H), 8.51 (s, 1H), 8.20 (d, J=2.07 Hz, 1H), 7.81 (s, 1H), 7.66 (d, J=2.25 Hz, 1H), 7.54 (d, J=9.01 Hz, 1H), 7.19 (dd, J=9.02, 2.28 Hz, 1H), 5.63 (t, J=3.77 Hz, 1H), 2.97 (dt, J=17.62, 4.66 Hz, 1H), 2.89-2.77 (m, 1H), 2.31-2.20 (m, 1H), 2.11-1.89 (m, 2H), 1.88-1.76 (m, 1H). MS-ESI (m/z) calc'd for C₂₀H₁₆N₅O₂ [M+H]⁺: 358.1. Found 358.2. A second fraction was isolated to afford 8-((3-(oxazol-5-yl)-1H-indazol-5-yl)oxy)-5,6,7,8-tetrahydroquinoline-3-carbonitrile, enantiomer 2 (5.9 mg, 1.2%). ¹H NMR (400 MHz, DMSO-d₆) δ 13.40 (s, 1H), 8.87 (d, J=2.05 Hz, 1H), 8.51 (s, 1H), 8.21 (d, J=2.07 Hz, 1H), 7.82 (s, 1H), 7.67 (d, J=2.24 Hz, 1H), 7.54 (d, J=9.00 Hz, 1H), 7.20 (dd, J=9.02, 2.31 Hz, 1H), 5.64 (t, J=3.77 Hz, 1H), 2.98 (dt, J=17.05, 4.42 Hz, 1H), 2.90-2.77 (m, 1H), 2.33-2.18 (m, 1H), 2.12-1.89 (m, 2H), 1.91-1.76 (m, 1H). MS-ESI (m/z) calc'd for C₂₀H₁₆N₅O₂ [M+H]⁺: 358.1. Found 358.2.

### Example 17: 5-((3-(Isoxazol-4-yl)-1H-indazol-5-yl)oxy)-5,6,7,8-tetrahydronaphthalene-2-carbonitrile, enantiomer 1 and 2

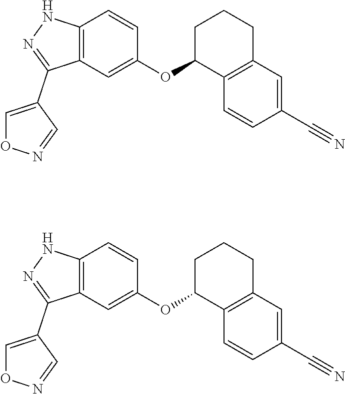 [SMILES: N#Cc1ccc2c(c1)CCC[C@@H]2Oc1ccc2[nH]nc(-c3cnoc3)c2c1 ; N#Cc1ccc2c(c1)CCC[C@H]2Oc1ccc2[nH]nc(-c3cnoc3)c2c1]

### Step 1: 5-Hydroxy-5,6,7,8-tetrahydronaphthalene-2-carbonitrile

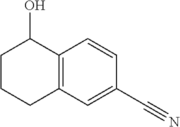 [SMILES: N#Cc1ccc2c(c1)CCCC2O]

A solution of 6-cyano-1-tetralone (250.0 mg, 1.46 mmol) in MeOH (24.86 mL) was cooled to 0° C. Sodium borohydride (110.48 mg, 2.92 mmol) was added and the resulting solution was warmed to r.t. and stirred for 30 min. Water was added and the aqueous layer was extracted with DCM. The combined organic layers were dried over Na₂SO₄, filtered and concentrated to afford the title compound (246.7 mg, 97%). ¹H NMR (400 MHz, CDCl₃) δ 7.58 (d, J=8.0 Hz, 1H), 7.51-7.46 (m, 1H), 7.40 (d, J=1.6 Hz, 1H), 4.79 (q, J=5.8 Hz, 1H), 2.90-2.81 (m, 1H), 2.75 (dt, J=17.2, 6.2 Hz, 1H), 2.14-2.05 (m, 1H), 2.04-1.94 (m, 1H), 1.92-1.79 (m, 2H), 1.77 (d, J=6.5 Hz, 1H). MS-ESI (m/z) calc'd for C₁₁H₁₂NO [M+H]⁺: 174.1. Found 174.0.

### Step 2: tert-Butyl 5-((6-cyano-1,2,3,4-tetrahydronaphthalen-1-yl)oxy)-3-iodo-1H-indazole-1-carboxylate

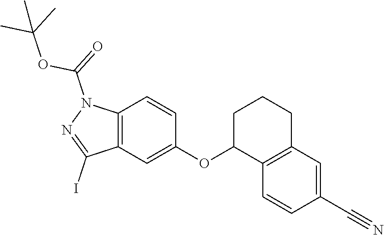 [SMILES: CC(C)(C)OC(=O)n1nc(I)c2cc(OC3CCCc4cc(C#N)ccc43)ccc21]

5-Hydroxy-5,6,7,8-tetrahydronaphthalene-2-carbonitrile (246.0 mg, 1.42 mmol) was dissolved in THF (2 mL) and stirred under N₂. tert-Butyl 5-hydroxy-3-iodo-1H-indazole-1-carboxylate (511.5 mg, 1.42 mmol) and triphenylphosphine (409.77 mg, 1.56 mmol) were added followed by dropwise addition of diethyl azodicarboxylate (0.22 mL, 1.42 mmol) at 0° C. The reaction was then stirred at room temperature for 2 hrs after which tert-butyl 5-hydroxy-3-iodo-1H-indazole-1-carboxylate (250 mg) and triphenylphosphine (200 mg) were added. After stirring for 1 hr, the resulting solution was partitioned between water and EtOAc. The organic layer was separated, washed with brine, dried over anhydrous Na₂SO₄, filtered and concentrated. The material was purified by column chromatography on silica gel using a 0-50% EtOAc/cyclohexane gradient eluent to afford the title compound (499 mg, 68%) as a white solid. ¹H NMR (400 MHz, DMSO-d₆) δ 7.99 (d, J=9.12 Hz, 1H), 7.69 (d, J=1.69 Hz, 1H), 7.64 (dd, J=7.94, 1.74 Hz, 1H), 1.64 (s, 10H), 7.55 (d, J=8.01 Hz, 1H), 7.41 (dd, J=9.12, 2.41 Hz, 1H), 7.17 (d, J=2.38 Hz, 1H), 5.72 (t, J=4.90 Hz, 1H), 2.91 (dt, J=17.19, 5.60 Hz, 1H), 2.85-2.71 (m, 1H), 2.02 (ddd, J=12.74, 8.51, 3.96 Hz, 2H), 1.95-1.74 (m, 1H). MS-ESI (m/z) calc'd for C₂₃H₂₃IN₃O₃ [M+H]⁺: 516.1. Found 516.3.

### Step 3: 5-((3-(Isoxazol-4-yl)-1H-indazol-5-yl)oxy)-5,6,7,8-tetrahydronaphthalene-2-carbonitrile

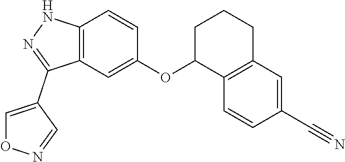 [SMILES: N#Cc1ccc2c(c1)CCCC2Oc1ccc2[nH]nc(-c3cnoc3)c2c1]

A microwave vial was charged with tert-butyl 5-[(6-cyano-1,2,3,4-tetrahydronaphthalen-1-yl)oxy]-3-iodoindazole-1-carboxylate (184.0 mg, 0.360 mmol), isoxazole-4-boronic acid (40.3 mg, 0.360 mmol), KOAc (70.08 mg, 0.710 mmol), 1,4-dioxane (4 mL) and water (1 mL). The vial was flushed with N₂ for 10 min, then Pd(amphos)Cl₂ (50.71 mg, 0.070 mmol) was added. The vial was sealed and irradiated at 100° C. for 30 min. The reaction mixture was partitioned between EtOAc and water and extracted. The organic layer was washed with water, dried over Na₂SO₄, filtered and concentrated. The material was purified by column chromatography on silica gel using a 0-80% gradient of EtOAc in cyclohexane to afford the title compound (65 mg, 51%) as an off-white solid.

### Step 4: 5-((3-(Isoxazol-4-yl)-1H-indazol-5-yl)oxy)-5,6,7,8-tetrahydronaphthalene-2-carbonitrile, enantiomer 1 and 2

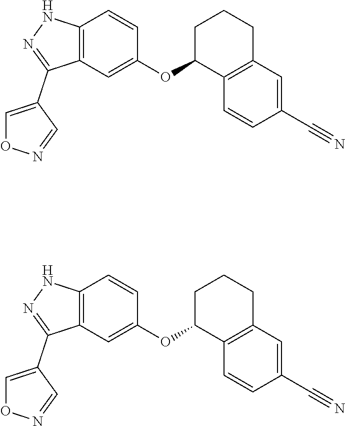 [SMILES: N#Cc1ccc2c(c1)CCC[C@@H]2Oc1ccc2[nH]nc(-c3cnoc3)c2c1 ; N#Cc1ccc2c(c1)CCC[C@H]2Oc1ccc2[nH]nc(-c3cnoc3)c2c1]

5-((3-(Isoxazol-4-yl)-1H-indazol-5-yl)oxy)-5,6,7,8-tetrahydronaphthalene-2-carbonitrile was subjected to chiral separation using Method S to afford 5-((3-(isoxazol-4-yl)-1H-indazol-5-yl)oxy)-5,6,7,8-tetrahydronaphthalene-2-carbonitrile, enantiomer 1 (18.9 mg, 15%). ¹H NMR (400 MHz, DMSO-d₆) δ 10.05 (t, J=5.67 Hz, 1H), 8.93 (s, 1H), 8.85 (s, 1H), 7.58-7.41 (m, 4H), 7.29 (d, J=2.20 Hz, 1H), 7.21 (dd, J=8.99, 2.23 Hz, 1H), 5.41-5.36 (m, 1H), 2.96 (dt, J=16.87, 5.58 Hz, 1H), 2.89-2.77 (m, 1H), 2.25-2.15 (m, 1H), 2.15-2.02 (m, 2H), 1.93-1.81 (m, 1H), MS-ESI (m/z) calc'd for C₂₁H₁₇N₄O₂ [M+H]⁺: 357.1. Found 357.1. A second fraction was isolated to afford 5-((3-(isoxazol-4-yl)-1H-indazol-5-yl)oxy)-5,6,7,8-tetrahydronaphthalene-2-carbonitrile, enantiomer 2 (17.8 mg, 14%). ¹H NMR (400 MHz, DMSO-d₆) δ 10.06 (s, 1H), 8.93 (s, 1H), 8.85 (s, 1H), 7.64-7.41 (m, 4H), 7.29 (d, J=2.20 Hz, 1H), 7.21 (dd, J=8.99, 2.25 Hz, 1H), 5.43-5.36 (m, 1H), 2.96 (dt, J=16.95, 5.58 Hz, 1H), 2.88-2.78 (m, 1H), 2.26-2.15 (m, 1H), 2.14-2.00 (m, 2H), 1.94-1.81 (m, 1H). MS-ESI (m/z) calc'd for C₂₁H₁₇N₄O₂ [M+H]⁺: 357.1. Found 357.1.

### Example 18: 5-((3-(Oxazol-5-yl)-1H-indazol-5-yl)oxy)-5,6,7,8-tetrahydronaphthalene-2-carbonitrile, enantiomer 1 and 2

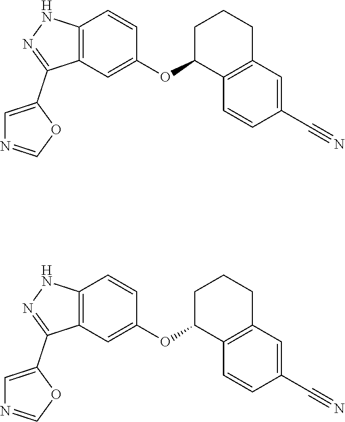 [SMILES: N#Cc1ccc2c(c1)CCC[C@@H]2Oc1ccc2[nH]nc(-c3cnco3)c2c1 ; N#Cc1ccc2c(c1)CCC[C@H]2Oc1ccc2[nH]nc(-c3cnco3)c2c1]

### Step 1: 5-((3-(Oxazol-5-yl)-1H-indazol-5-yl)oxy)-5,6,7,8-tetrahydronaphthalene-2-carbonitrile

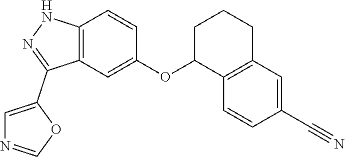 [SMILES: N#Cc1ccc2c(c1)CCCC2Oc1ccc2[nH]nc(-c3cnco3)c2c1]

A microwave vial was charged with tert-butyl 5-[(6-cyano-1,2,3,4-tetrahydronaphthalen-1-yl)oxy]-3-iodoindazole-1-carboxylate (218.0 mg, 0.420 mmol), Pd(amphos)Cl₂ (30.04 mg, 0.040 mmol), 5-(4,4,5,5-tetramethyl-1,3,2-dioxaborolan-2-yl)-1,3-oxazole (90.75 mg, 0.470 mmol), 1,4-dioxane (4 mL) and water (1 mL). The vial was flushed with N₂ for 15 min, then KOAc (74.73 mg, 0.760 mmol) was added. The vial was sealed and irradiated at 100° C. for 30 min. The material was partitioned between water and EtOAc and extracted. The combined organic layers were washed with brine, dried with Na₂SO₄, filtered and evaporated to dryness. The material was purified by column chromatography on silica gel using a 20-30% EtOAc/cyclohexane gradient eluent to afford the title compound (106 mg, 70%).

### Step 2: 5-((3-(Oxazol-5-yl)-1H-indazol-5-yl)oxy)-5,6,7,8-tetrahydronaphthalene-2-carbonitrile, enantiomer 1 and 2

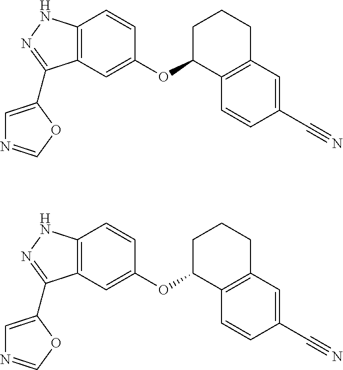 [SMILES: N#Cc1ccc2c(c1)CCC[C@@H]2Oc1ccc2[nH]nc(-c3cnco3)c2c1 ; N#Cc1ccc2c(c1)CCC[C@H]2Oc1ccc2[nH]nc(-c3cnco3)c2c1]

5-((3-(Oxazol-5-yl)-1H-indazol-5-yl)oxy)-5,6,7,8-tetrahydronaphthalene-2-carbonitrile was subjected to chiral separation using Method T to afford 5-((3-oxazol-5-yl)-1H-indazol-5-yl)oxy)-5,6,7,8-tetrahydronaphthalene-2-carbonitrile, enantiomer 1 (38.9 mg, 26%). ¹H NMR (400 MHz, DMSO-d₆) δ 13.39 (s, 1H), 8.50 (s, 1H), 7.85 (s, 1H), 7.72-7.48 (m, 5H), 7.20 (dd, J=9.01, 2.28 Hz, 1H), 5.68 (t, J=4.91 Hz, 1H), 2.91 (dt, J=17.13, 5.66 Hz, 1H), 2.86-2.73 (m, 1H), 2.02 (q, J=5.40 Hz, 2H), 1.98-1.86 (m, 1H), 1.87-1.75 (m, 1H), MS-ESI (m/z) calc'd for C₂₁H₁₇N₄O₂ [M+H]⁺: 357.1. Found 357.1. A second fraction was isolated to afford 5-((3-(oxazol-5-yl)-1H-indazol-5-yl)oxy)-5,6,7,8-tetrahydronaphthalene-2-carbonitrile, enantiomer 2 (38.8 mg, 26%). ¹H NMR (400 MHz, DMSO-d₆) δ 13.40 (s, 1H), 8.50 (s, 1H), 7.85 (s, 1H), 7.72-7.46 (m, 5H), 7.20 (dd, J=9.01, 2.23 Hz, 1H), 5.68 (t, J=4.92 Hz, 1H), 2.91 (dt, J=17.19, 5.66 Hz, 1H), 2.85-2.74 (m, 1H), 2.03 (q, J=5.42 Hz, 2H), 1.98-1.86 (m, 1H), 1.86-1.75 (m, 1H). MS-ESI (m/z) ca'lc'd for C₂₁H₁₇N₄O₂ [M+H]⁺: 357.1. Found 357.1.

### Example 19: 5-((3-Methyl-1H-indazol-5-yl)oxy)-5,6,7,8-tetrahydronaphthalene-2-carbonitrile, enantiomer 1 and 2

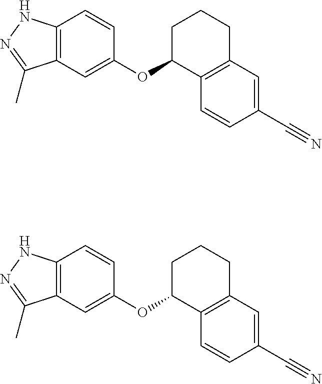 [SMILES: Cc1n[nH]c2ccc(O[C@H]3CCCc4cc(C#N)ccc43)cc12 ; Cc1n[nH]c2ccc(O[C@@H]3CCCc4cc(C#N)ccc43)cc12]

### Step 1: tert-Butyl 5-bromo-3-methyl-1H-indazole-1-carboxylate

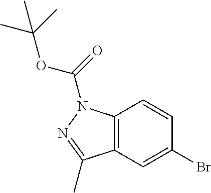 [SMILES: Cc1nn(C(=O)OC(C)(C)C)c2ccc(Br)cc12]

5-Bromo-3-methyl-1H-indazole (1.84 g, 8.72 mmol) and DMAP (11.0 mg, 0.090 mmol) were dissolved in DCM (36 mL). Di-tert-butyl dicarbonate (2.09 g, 9.59 mmol) was added and the mixture was stirred at r.t. for 3 hrs and then concentrated. The residue was diluted with EtOAc and washed with 1 N NaOH, 0.1 N HCl, and brine. The organic layer was dried over Na₂SO₄ and filtered. The filtrate was concentrated under reduced pressure to afford the title compound (2.715 g, 100%). ¹H NMR (400 MHz, CDCl₃) δ 8.02 (d, J=8.80 Hz, 1H), 7.86-7.77 (m, 1H), 7.67-7.58 (m, 1H), 2.66-2.55 (m, 3H), 1.79-1.74 (m, 9H). MS-ESI (m/z) calc'd for C₁₃H₁₆BrN₂O₂ [M+H]⁺: 311.0, 313.1. Found 312.2.

### Step 2: tert-Butyl 3-methyl-5-(4,4,5,5-tetramethyl-1,3,2-dioxaborolan-2-yl)-1H-indazole-1-carboxylate

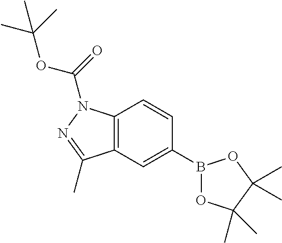 [SMILES: Cc1nn(C(=O)OC(C)(C)C)c2ccc(B3OC(C)(C)C(C)(C)O3)cc12]

To a solution of tert-butyl 5-bromo-3-methyl-1H-indazole-1-carboxylate (2.72 g, 8.73 mmol) in 1,4-dioxane (69 mL) was added Pd(dppf)Cl₂ (0.64 g, 0.870 mmol), 4,4,5,5-tetramethyl-2-(4,4,5,5-tetramethyl-1,3,2-dioxaborolan-2-yl)-1,3,2-dioxaborolane (4.43 g, 17.45 mmol) and KOAc (1.71 g, 17.45 mmol). The reaction mixture was stirred at 80° C. for 2 hrs and the mixture was filtered and concentrated to afford the title compound (3.13, 100%). MS-ESI (m/z) calc'd for C₁₉H₂₈BN₂O₄ [M+H]⁺: 359.2. Found 359.4.

### Step 3: tert-Butyl 5-hydroxy-3-methyl-1H-indazole-1-carboxylate

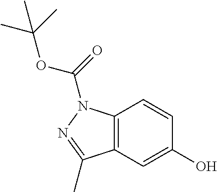 [SMILES: Cc1nn(C(=O)OC(C)(C)C)c2ccc(O)cc12]

To a solution of tert-butyl 3-methyl-5-(4,4,5,5-tetramethyl-1,3,2-dioxaborolan-2-yl)indazole-1-carboxylate (3.13 g, 8.73 mmol) in MeOH (25 mL) was added hydrogen peroxide (4.37 g, 45 mmol) and the mixture was stirred at r.t. for 3 days. The reaction was quenched with saturated aqueous Na₂SO₃ and then partitioned between water and EtOAc and extracted. The combined organic phases were washed with brine, dried over Na₂SO₄, filtered and concentrated. The residue was purified by column chromatography on silica gel using a 0-50% EtOAc/cyclohexane gradient eluent to afford the title compound (2.2 g, 98%). ¹H NMR (400 MHz, CDCl₃) δ ppm 7.99 (d, J=8.80 Hz, 1H), 7.10 (dd, J=9.02, 2.42 Hz, 1H), 7.05-7.00 (m, 1H), 5.47 (s, 1H), 2.62-2.51 (m, 3H), 1.72-1.79 (m, 9H). MS-ESI (m/z) calc'd for C₁₉H₂₈BN₂O₄ [M+H]⁺: 249.1. Found 249.2.

### Step 4: tert-Butyl 5-((6-cyano-1,2,3,4-tetrahydronaphthalen-1-yl)oxy)-3-methyl-1H-indazole-1-carboxylate

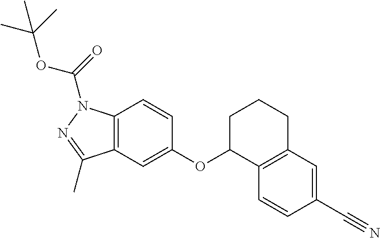 [SMILES: Cc1nn(C(=O)OC(C)(C)C)c2ccc(OC3CCCc4cc(C#N)ccc43)cc12]

A solution of 5-hydroxy-5,6,7,8-tetrahydronaphthalene-2-carbonitrile (107.0 mg, 0.620 mmol), tert-butyl 5-hydroxy-3-methyl-1H-indazole-1-carboxylate (213.02 mg, 0.620 mmol) and triphenylphosphine (178.23 mg, 0.680 mmol) in 1 mL of THF was stirred at −10° C. Diethyl azodicarboxylate (0.1 mL, 0.620 mmol) was then added dropwise and the reaction mixture was allowed to reach r.t. After 1 hr, an additional 0.5 eq of triphenylphosphine and diethyl azodicarboxylate were added. After stirring for 1 hr the resulting solution was diluted with water and EtOAc. The organic layers were separated, combined, washed with brine, dried over Na₂SO₄, filtered and concentrated. The material was purified by column chromatography on silica gel using a 0-20% EtOAc/cyclohexane gradient eluent to afford the title compound (75.5 mg, 30%). MS-ESI (m/z) calc'd for C₁₉H₂₈BN₂O₄ [M+H]⁺: 404.2. Found 404.5.

### Step 5: 5-((3-Methyl-1H-indazol-5-yl)oxy)-5,6,7,8-tetrahydronaphthalene-2-carbonitrile

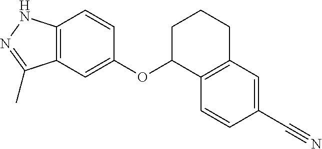 [SMILES: Cc1n[nH]c2ccc(OC3CCCc4cc(C#N)ccc43)cc12]

A solution of tert-butyl 5-((6-cyano-1,2,3,4-tetrahydronaphthalen-1-yl)oxy)-3-methyl-1H-indazole-1-carboxylate (75.5 mg, 0.190 mmol) and trifluoroacetic acid (300.0 mL, 3917.7 mmol) in DCM (1 mL) was stirred at r.t. overnight. The reaction mixture was concentrated. The material was purified by reversed phase chromatography on using a 0-60% MeCN/H₂O (0.1% formic acid) gradient eluent to afford the title compound (49 mg, 86%).

### Step 6: 5-((3-Methyl-1H-indazol-5-yl)oxy)-5,6,7,8-tetrahydronaphthalene-2-carbonitrile, enantiomer 1 and 2

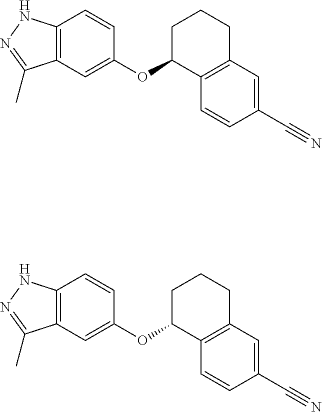 [SMILES: Cc1n[nH]c2ccc(O[C@H]3CCCc4cc(C#N)ccc43)cc12 ; Cc1n[nH]c2ccc(O[C@@H]3CCCc4cc(C#N)ccc43)cc12]

5-((3-Methyl-1H-indazol-5-yl)oxy)-5,6,7,8-tetrahydronaphthalene-2-carbonitrile was subjected to chiral separation using Method U to afford: 5-((3-methyl-1H-indazol-5-yl)oxy)-5,6,7,8-tetrahydronaphthalene-2-carbonitrile, enantiomer 1 (14.6 mg, 26%). ¹H NMR (400 MHz, methanol-d₄) δ 7.61-7.51 (m, 3H), 7.42 (d, J=9.0 Hz, 1H), 7.32 (d, J=2.3 Hz, 1H), 7.15 (dd, J=9.0, 2.3 Hz, 1H), 5.48 (t, J=4.8 Hz, 1H), 3.03-2.79 (m, 2H), 2.54 (s, 3H), 2.21-2.00 (m, 3H), 1.96-1.82 (m, 1H). MS-ESI (m/z) calc'd for C₁₉H₁₈N₃O [M+H]⁺: 304.1. Found 304.2. A second fraction was isolated to afford 5-((3-methyl-1H-indazol-5-yl)oxy)-5,6,7,8-tetrahydronaphthalene-2-carbonitrile, enantiomer 2 (14.3 mg, 25%). ¹H NMR (400 MHz, methanol-d₄) δ 7.60-7.49 (m, 3H), 7.42 (dd, J=8.9, 0.7 Hz, 1H), 7.32 (d, J=2.2 Hz, 1H), 7.15 (dd, J=9.0, 2.3 Hz, 1H), 5.48 (t, J=4.9 Hz, 1H), 3.04-2.79 (m, 2H), 2.54 (s, 3H), 2.22-1.99 (m, 3H), 1.94-1.80 (m, 1H). MS-ESI (m/z) calc'd for C₁₉H₁₈N₃O [M+H]⁺: 304.1. Found 304.2.

### Example 20: 1-((3-(Oxazol-5-yl)-1H-pyrazolo[3,4-c]pyridin-5-yl)oxy)-2,3-dihydro-1H-indene-5-carbonitrile, enantiomer 1 and 2

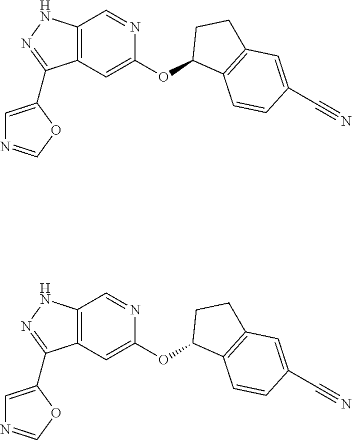 [SMILES: N#Cc1ccc2c(c1)CC[C@@H]2Oc1cc2c(-c3cnco3)n[nH]c2cn1 ; N#Cc1ccc2c(c1)CC[C@H]2Oc1cc2c(-c3cnco3)n[nH]c2cn1]

### Step 1: 1-Hydroxy-2,3-dihydro-1H-indene-5-carbonitrile

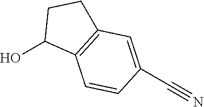 [SMILES: N#Cc1ccc2c(c1)CCC2O]

To a stirred solution of 1-oxo-2,3-dihydro-1H-indene-5-carbonitrile (3.4 g, 21.63 mmol) in MeOH (22 mL) was added NaBH₄ (982.11 mg, 25.96 mmol) gradually over 0.5 hr at 0° C. The reaction mixture was then warmed to 20° C. and stirred for 2 hrs. The reaction mixture was concentrated and the residue was dissolved in EtOAc (50 mL) and extracted with water. The organic phase was washed with saturated aqueous NaHCO₃, washed with brine, dried over Na₂SO₄, filtered and concentrated. The residue was purified by silica gel chromatography using 5-50% EtOAc/petroleum ether gradient eluent to afford the title compound (2.67 g, 77%) as a white solid.

### Step 2: 5-Bromo-1H-pyrazolo[3,4-c]pyridine-3-carbaldehyde

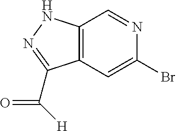 [SMILES: O=Cc1n[nH]c2cnc(Br)cc12]

To a solution of NaNO₂ (2.80 g, 40.60 mmol) in H₂O (8 mL) at 0° C. was added HCl (2 M, 17.76 mL) slowly and the resulting mixture was kept under N₂ for 10 mins before adding DMF (18 mL). Then a solution of 5-bromo-1H-pyrazolo[3,4-c]pyridine (1 g, 5.08 mmol) in DMF (18 mL) was added at 0° C. The reaction mixture was heated at 80° C. and stirred for 6 hrs under N₂. The resulting mixture was adjusted to pH=8 with saturated aqueous NaHCO₃. The aqueous phase was extracted with EtOAc and the combined organic phases were dried over Na₂SO₄, filtered and concentrated to afford the title compound (1 g) as a brown oil, which was used without further purification.

### Step 3: 5-Bromo-1-((2-(trimethylsilyl)ethoxy)methyl)-1H-pyrazolo[3,4-c]pyridine-3-carbaldehyde

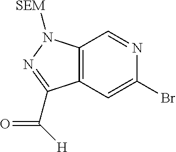 [SMILES: C[Si](C)(C)CCOCn1nc(C=O)c2cc(Br)ncc21]

To a stirred solution of 5-bromo-1H-pyrazolo[3,4-c]pyridine-3-carbaldehyde (1 g, 4.42 mmol) in THF (20 mL) at 20° C. was added N-cyclohexyl-N-methylcyclohexanamine (1.73 g, 8.85 mmol) and SEM-Cl (1.11 g, 6.64 mmol). The reaction mixture was then stirred at 20° C. for 12 hrs. The reaction mixture was poured into water (20 mL) and the aqueous phase was extracted with EtOAc. The combined organic phases were washed with brine, dried over Na₂SO₄, filtered and concentrated in vacuum. The residue was purified by silica gel chromatography using a 0-20% EtOAc/petroleum ether gradient eluent to afford the title compound (1 g, 63%) as a light yellow oil.

### Step 4: 5-(5-Bromo-1-((2-(trimethylsilyl)ethoxy)methyl)-1H-pyrazolo[3,4-c]pyridin-3-yl)oxazole

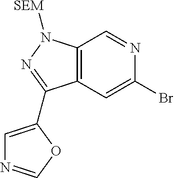 [SMILES: C[Si](C)(C)CCOCn1nc(-c2cnco2)c2cc(Br)ncc21]

To a stirred solution of 5-bromo-1-((2-(trimethylsilyl)ethoxy)methyl)-1H-pyrazolo[3,4-c]pyridine-3-carbaldehyde (1 g, 2.81 mmol) in MeOH (16 mL) was added K₂CO₃ (775.81 mg, 5.61 mmol) and TosMIC (602.77 mg, 3.09 mmol) at 20° C. The reaction mixture was then heated to 80° C. and stirred for 1 hr. After cooling to 20° C., the reaction mixture was concentrated and purified silica gel chromatography using a 0-20% EtOAc/petroleum ether gradient eluent to afford the title compound (1 g, 90%) as a light yellow oil.

### Step 5: 3-(Oxazol-5-yl)-1-((2-(trimethylsilyl)ethoxy)methyl)-1H-pyrazolo[3,4-c]pyridin-5-ol

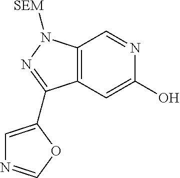 [SMILES: C[Si](C)(C)CCOCn1nc(-c2cnco2)c2cc(O)ncc21]

5-(5-Bromo-1-((2-(trimethylsilyl)ethoxy)methyl)-1H-pyrazolo[3,4-c]pyridin-3-yl)oxazole (100 mg, 252.96 umol), KOH (85.15 mg, 1.52 mmol), Pd₂(dba)₃ (11.58 mg, 12.65 umol) and t-Bu Xphos (10.74 mg, 25.30 umol) in dioxane (2 mL) and H₂O (2 mL) was degassed at 20° C. and then heated to 60° C. for 12 hrs under N₂. After cooling to 20° C., the reaction mixture was filtered and the filtrate was adjusted to pH=5 with AcOH. The filtrate was diluted with water (5 mL) and extracted with EtOAc. The combined organic phases were washed with brine, dried over Na₂SO₄, filtered and concentrated. The residue was purified by preparative-TLC (SiO₂, Rf=0.50) using 25% EtOH/EtOAc gradient eluent to afford the title compound (50 mg) as a yellow solid.

### Step 6: 1-((3-(Oxazol-5-yl)-1-((2-(trimethylsilyl)ethoxy)methyl)-1H-pyrazolo[3,4-c]pyridin-5-yl)oxy)-2,3-dihydro-1H-indene-5-carbonitrile

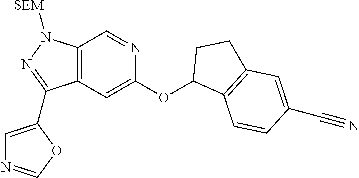 [SMILES: C[Si](C)(C)CCOCn1nc(-c2cnco2)c2cc(OC3CCc4cc(C#N)ccc43)ncc21]

To a stirred solution of 3-(oxazol-5-yl)-1-((2-(trimethylsilyl)ethoxy)methyl)-1H-pyrazolo[3,4-c]pyridin-5-ol (70 mg, 210.57 umol) and 1-hydroxy-2,3-dihydro-1H-indene-5-carbonitrile (36.87 mg, 231.63 umol) in toluene (5 mL) was added n-Bu₃P (85.20 mg, 421.14 umol) and 1,1′-(azodicarbonyl)dipiperidine (106.26 mg, 421.14 umol) at 0° C. The reaction mixture was stirred at 60° C. for 12 hrs under N₂. After cooling to 20° C., the reaction mixture was concentrated. The residue was diluted with water and extracted with EtOAc. The combined organic phases were concentrated and the residue was purified by preparative TLC (SiO₂, Rf=0.43) using 30% EtOAc/petroleum ether as eluent to afford the title compound (60 mg, 60%) as a yellow oil.

### Step 7: 1-((3-(Oxazol-5-yl)-1H-pyrazolo[3,4-c]pyridin-5-yl)oxy)-2,3-dihydro-1H-indene-5-carbonitrile

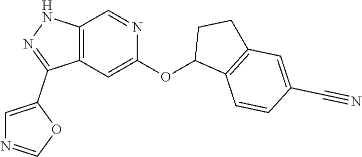 [SMILES: N#Cc1ccc2c(c1)CCC2Oc1cc2c(-c3cnco3)n[nH]c2cn1]

To a stirred solution of 1-((3-(oxazol-5-yl)-1-((2-(trimethylsilyl)ethoxy)methyl)-1H-pyrazolo[3,4-c]pyridin-5-yl)oxy)-2,3-dihydro-1H-indene-5-carbonitrile (40 mg, 84.46 umol) and ethane-1,2-diamine (15.23 mg, 253.38 umol) in THF (2 mL) was added a 1 M solution of TBAF in THF (422.30 uL, 422.3 umol) under N₂ at 20° C. The reaction mixture was heated to 60° C. and stirred for 12 hrs. The reaction mixture was purified by preparative TLC (SiO₂, Rf=0.43) using 20% EtOH/EtOAc gradient eluent. The obtained material was then further purified by SFC conditions using Method HL to afford the title compound (8 mg, 30%).

### Step 8: 1-((3-(Oxazol-5-yl)-1H-pyrazolo[3,4-c]pyridin-5-yl)oxy)-2,3-dihydro-1H-indene-5-carbonitrile, enantiomer 1 and 2

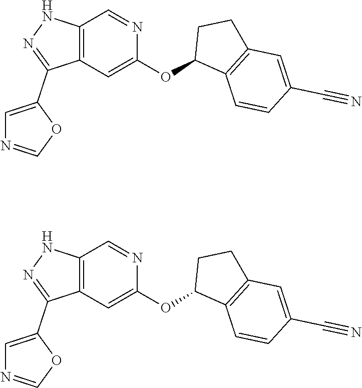 [SMILES: N#Cc1ccc2c(c1)CC[C@@H]2Oc1cc2c(-c3cnco3)n[nH]c2cn1 ; N#Cc1ccc2c(c1)CC[C@H]2Oc1cc2c(-c3cnco3)n[nH]c2cn1]

1-((3-(Oxazol-5-yl)-1H-pyrazolo[3,4-c]pyridin-5-yl)oxy)-2,3-dihydro-1H-indene-5-carbonitrile was subjected to chiral separation using Method V to afford 1-((3-(oxazol-5-yl)-1H-pyrazolo[3,4-c]pyridin-5-yl)oxy)-2,3-dihydro-1H-indene-5-carbonitrile, enantiomer 1 (4 mg, 14%) as a white solid. ¹H NMR (400 MHz, DMSO-d₆) δ 8.85 (s, 1H), 8.51 (s, 1H), 7.87 (s, 1H), 7.82 (s, 1H), 7.69-7.63 (m, 1H), 7.61-7.56 (m, 1H), 7.39 (s, 1H), 6.55 (dd, J=5.2, 6.7 Hz, 1H), 3.18-3.07 (m, 1H), 3.03-2.90 (m, 1H), 2.72-2.59 (m, 1H), 2.22-2.08 (m, 1H). MS-ESI (m/z) calc'd for C₁₉H₁₄N₅O₂ [M+H]⁺: 344.1. Found 344.2. A second fraction was isolated to afford 1-((3-(oxazol-5-yl)-1H-pyrazolo[3,4-c]pyridin-5-yl)oxy)-2,3-dihydro-1H-indene-5-carbonitrile, enantiomer 2 (4 mg, 13%) as a white solid. ¹H NMR (400 MHz, DMSO-d₆) δ ppm 8.85 (s, 1H), 8.51 (s, 1H), 7.87 (s, 1H), 7.81 (s, 1H), 7.69-7.63 (m, 1H), 7.61-7.54 (m, 1H), 7.38 (s, 1H), 6.59-6.49 (m, 1H), 3.18-3.06 (m, 1H), 3.03-2.90 (m, 1H), 2.71-2.59 (m, 1H), 2.21-2.09 (m, 1H). MS-ESI (m/z) calc'd for C₁₉H₁₄N₅O₂ [M+H]⁺: 344.1. Found 344.2.

### Example 21: 1-((3-(Oxazol-5-yl)-1H-pyrazolo[3,4-c]pyridin-5-yl)amino)-2,3-dihydro-1H-indene-5-carbonitrile, enantiomer 1 and 2

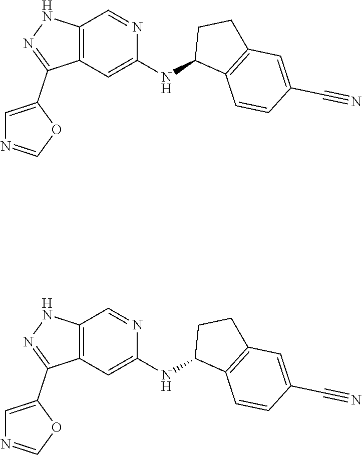 [SMILES: N#Cc1ccc2c(c1)CC[C@@H]2Nc1cc2c(-c3cnco3)n[nH]c2cn1 ; N#Cc1ccc2c(c1)CC[C@H]2Nc1cc2c(-c3cnco3)n[nH]c2cn1]

### Step 1: 1-Oxo-2,3-dihydro-1H-indene-5-carbonitrile

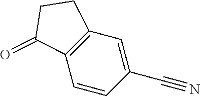 [SMILES: N#Cc1ccc2c(c1)CCC2=O]

A mixture of 5-bromo-2,3-dihydro-1H-inden-1-one (5 g, 23.69 mmol), CuCN (2.55 g, 28.43 mmol) in 50 mL of DMF at 20° C. was degassed and purged with N₂ (3×) and then the mixture was stirred at 145° C. for 12 hrs under N₂. The reaction mixture was concentrated and purified by silica gel chromatography using a 0-6% EtOAc/petroleum ether gradient eluent to afford the title compound (1.3 g, 35%) as a yellow solid.

### Step 2: 1-Amino-2,3-dihydro-1H-indene-5-carbonitrile

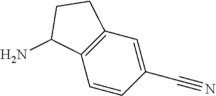 [SMILES: N#Cc1ccc2c(c1)CCC2N]

To a solution of 1-oxo-2,3-dihydro-1H-indene-5-carbonitrile (0.9 g, 5.73 mmol) in 10 mL of MeOH was added NH₄OAc (6.62 g, 85.90 mmol) in a microwave vial. The mixture was stirred at 20° C. for 10 min. Then the NaBH₃CN (1.44 g, 22.91 mmol) was added to the mixture. The vial was sealed and irradiated at 90° C. for 30 min. The procedure was repeated with a second 0.45 g batch and the mixtures were combined and filtered. The filtrate was concentrated and the residue was diluted with 200 mL of H₂O and acidified with 1N HCl to pH=3. The aqueous phase was then extracted with EtOAc and the organic phase was discarded. The aqueous phase was basified with addition of solid NaHCO₃ to pH=8 and extracted with DCM. The combined organic phases were dried over Na₂SO₄ to afford the title compound (330 mg, 37%) as a green oil.

### Step 3: 1-((3-(Oxazol-5-yl)-1-((2-(trimethylsilyl)ethoxy)methyl)-1H-pyrazolo[3,4-c]pyridin-5-yl)amino)-2,3-dihydro-1H-indene-5-carbonitrile

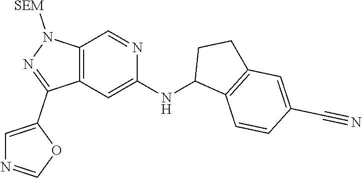 [SMILES: C[Si](C)(C)CCOCn1nc(-c2cnco2)c2cc(NC3CCc4cc(C#N)ccc43)ncc21]

A mixture of 1-amino-2,3-dihydro-1H-indene-5-carbonitrile (100 mg, 632.11 umol), 5-(5-bromo-1-((2-(trimethylsilyl)ethoxy)methyl)-1H-pyrazolo[3,4-c]pyridin-3-yl)oxazole (124.95 mg, 316.06 umol), a 2 M solution of sodium tert-butoxide in THF (2 M, 316.06 uL) and tBuXPhos-Pd-G3 (25.11 mg, 31.61 umol) in 4 mL of THF was degassed and purged with N₂ (3×) at 20° C. The mixture was then stirred at 80° C. for 6 hrs under N₂. The reaction mixture was concentrated and purified by silica gel chromatography using a 0-31% EtOAc/petroleum ether gradient eluent to afford the title compound (50 mg, 17%) as a yellow oil.

### Step 4: 1-((1-(Hydroxymethyl)-3-(oxazol-5-yl)-1H-pyrazolo[3,4-c]pyridin-5-yl)amino)-2,3-dihydro-1H-indene-5-carbonitrile

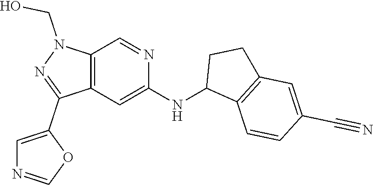 [SMILES: N#Cc1ccc2c(c1)CCC2Nc1cc2c(-c3cnco3)nn(CO)c2cn1]

A solution of 1-((3-(oxazol-5-yl)-1-((2-(trimethylsilyl)ethoxy)methyl)-1H-pyrazolo[3,4-c]pyridin-5-yl)amino)-2,3-dihydro-1H-indene-5-carbonitrile (60 mg, 126.95 umol) in 3 mL of TFA was stirred at 20° C. for 1 hr. The reaction mixture was concentrated under reduced pressure to afford the title compound (100 mg, TFA salt) as a yellow oil which was used without further purification.

### Step 5: 1-((3-(Oxazol-5-yl)-1H-pyrazolo[3,4-c]pyridin-5-yl)amino)-2,3-dihydro-1H-indene-5-carbonitrile

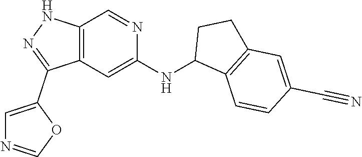 [SMILES: N#Cc1ccc2c(c1)CCC2Nc1cc2c(-c3cnco3)n[nH]c2cn1]

To a solution of 1-((1-(hydroxymethyl)-3-(oxazol-5-yl)-1H-pyrazolo[3,4-c]pyridin-5-yl)amino)-2,3-dihydro-1H-indene-5-carbonitrile (100 mg, 268.54 umol, TFA salt) in 3 mL of dioxane was added 25% solution of NH₄OH (564.75 mg, 4.03 mmol). The mixture was stirred at 20° C. for 12 hrs. The reaction mixture was concentrated under reduced pressure and the residue was purified by Method W to afford the title compound (9 mg, 10%) as a pale yellow solid.

### Step 6: 1-((3-(Oxazol-5-yl)-1H-pyrazolo[3,4-c]pyridin-5-yl)amino)-2,3-dihydro-1H-indene-5-carbonitrile, enantiomer 1 and 2

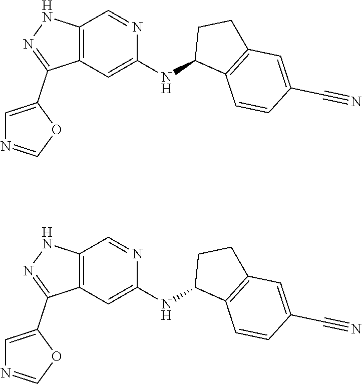 [SMILES: N#Cc1ccc2c(c1)CC[C@@H]2Nc1cc2c(-c3cnco3)n[nH]c2cn1 ; N#Cc1ccc2c(c1)CC[C@H]2Nc1cc2c(-c3cnco3)n[nH]c2cn1]

1-((3-(Oxazol-5-yl)-1H-pyrazolo[3,4-c]pyridin-5-yl)amino)-2,3-dihydro-1H-indene-5-carbonitrile was subjected to chiral separation using Method X to afford 1-((3-(oxazol-5-yl)-1H-pyrazolo[3,4-c]pyridin-5-yl)amino)-2,3-dihydro-1H-indene-5-carbonitrile, enantiomer 1 (4.55 mg, 5%) as a pale yellow solid. ¹H NMR (400 MHz, CDCl₃) δ 10.44 (br s, 1H), 8.70 (s, 1H), 8.04 (s, 1H), 7.58 (s, 2H), 7.53-7.44 (m, 2H), 6.96 (s, 1H), 5.45 (q, J=7.8 Hz, 1H), 4.76 (br d, J=8.3 Hz, 1H), 3.14-2.94 (m, 2H), 2.79 (qd, J=3.8, 16.2 Hz, 1H), 1.99 (qd, J=8.6, 12.7 Hz, 1H). MS-ESI (m/z) calc'd for C₁₉H₁₄N₅O₂ [M+H]⁺: 343.1. Found 343.1. A second fraction was isolated to afford 1-((3-(oxazol-5-yl)-1H-pyrazolo[3,4-c]pyridin-5-yl)amino)-2,3-dihydro-1H-indene-5-carbonitrile, enantiomer 2 (4.06 mg, 4%) as a pale yellow solid. ¹H NMR (400 MHz, CDCl₃) δ 10.43 (br s, 1H), 8.70 (s, 1H), 8.04 (s, 1H), 7.58 (s, 2H), 7.53-7.42 (m, 2H), 6.96 (s, 1H), 5.45 (q, J=7.5 Hz, 1H), 4.76 (br d, J=8.1 Hz, 1H), 3.17-2.93 (m, 2H), 2.86-2.70 (m, 1H), 1.99 (qd, J=8.6, 12.7 Hz, 1H). MS-ESI (m/z) calc'd for C₁₉H₁₄N₅O₂ [M+H]⁺: 343.1. Found 343.1.

### Example 22: 4-Methyl-1-((3-(oxazol-5-yl)-1H-indazol-5-yl)oxy)-2,3-dihydro-1H-indene-5-carbonitrile, enantiomer 1 and 2

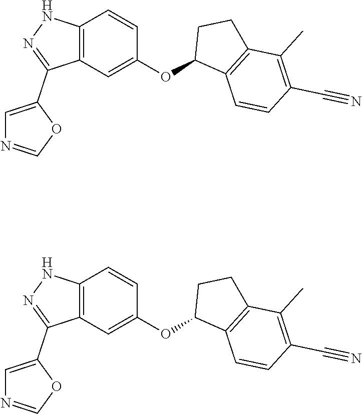 [SMILES: Cc1c(C#N)ccc2c1CC[C@@H]2Oc1ccc2[nH]nc(-c3cnco3)c2c1 ; Cc1c(C#N)ccc2c1CC[C@H]2Oc1ccc2[nH]nc(-c3cnco3)c2c1]

### Step 1: 5-Bromo-4-methyl-2,3-dihydro-1H-inden-1-ol

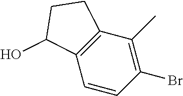

To a solution of 4-bromo-5-methyl-2,3-dihydro-1H-inden-1-one (200 mg, 888.57 umol) in 1 mL of EtOH was added NaBH₄ (50.42 mg, 1.33 mmol) at 20° C. The mixture was then stirred at 60° C. for 10 min. The reaction mixture was concentrated under reduced pressure and the residue was diluted with H₂O (5 mL) and extracted with EtOAc. The combined organic layers were dried over Na₂SO₄, filtered and concentrated under reduced pressure to afford the title compound (200 mg) as a white solid which was used without further purification.

### Step 2: 1-Hydroxy-4-methyl-2,3-dihydro-1H-indene-5-carbonitrile

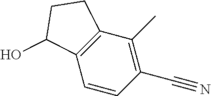

A mixture of 5-bromo-4-methyl-2,3-dihydro-1H-inden-1-ol (110 mg, 484.37 umol), Zn(CN)₂ (85.32 mg, 726.56 umol), Pd₂(dba)₃ (44.35 mg, 48.44 umol), dppf (26.85 mg, 48.44 umol) and Zn (3.17 mg, 48.44 umol) in 2 mL of DMA was degassed and purged with N₂ (3×) at 20° C. The mixture was then stirred at 120° C. for 2 hrs under an N₂ atmosphere in a microwave reactor. The mixture was diluted with H₂O (10 mL) and extracted with EtOAc. The combined organic layers were washed with brine, dried over Na₂SO₄, filtered and concentrated under reduced pressure to give a residue. The residue was purified by preparative TLC (SiO₂, Rf=0.40) using a 50% EtOAc/petroleum ether gradient eluent to afford the title compound (40 mg, 42%) as a pale yellow solid.

### Step 3: 5-Bromo-1H-indazole-3-carbaldehyde

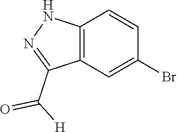 [SMILES: O=Cc1n[nH]c2ccc(Br)cc12]

To a solution of NaNO₂ (8.45 g, 122.42 mmol) in 10 mL of H₂O was added 5-bromo-1H-indazole (3 g, 15.30 mmol) in 60 mL of ACN slowly at 0° C. HCl (2 M, 36.04 mL) was then added to the mixture slowly at 0° C. The mixture was stirred at 25° C. for 5 hrs. The solution was concentrated and the solid was collected by filtration and transferred to a flask. DCM (80 mL) was added and stirred for 30 min at 0° C., the solid was filtered and concentrated to afford the title compound (1.8 g) as a brown solid which was used without further purification.

### Step 4: 5-Bromo-1-(tetrahydro-2H-pyran-2-yl)-1H-indazole-3-carbaldehyde

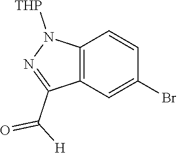

To a solution of 5-bromo-1H-indazole-3-carbaldehyde (1.78 g, 7.93 mmol) in 50 mL of CHCl₃ was added MsOH (76.19 mg, 792.75 umol) and DHP (2.00 g, 23.78 mmol) at 20° C. Then the mixture was stirred at 70° C. for 12 hrs. The mixture was concentrated and purified by silica gel chromatography using a 0-9% EtOAc/petroleum ether gradient eluent to afford the title compound (930 mg, 38%) as a yellow solid.

### Step 5: 5-(5-Bromo-1-(tetrahydro-2H-pyran-2-yl)-1H-indazol-3-yl)oxazole

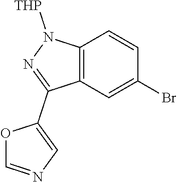

To a solution of 5-bromo-1-(tetrahydro-2H-pyran-2-yl)-1H-indazole-3-carbaldehyde (930 mg, 3.01 mmol), TosMIC (646.04 mg, 3.31 mmol) in 15 mL of MeOH was added K₂CO₃ (623.62 mg, 4.51 mmol). The mixture was stirred at 20° C. for 2 hrs. The reaction mixture was filtered and the filtrate was concentrated under reduced pressure to give a residue. The residue was diluted with EtOAc (20 mL) and filtered. The filtrate was concentrated and purified by silica gel chromatography using a 0-12% EtOAc/petroleum ether gradient to afford the title compound (860 mg, 82%) as a white solid.

### Step 6: 5-(1-(Tetrahydro-2H-pyran-2-yl)-5-(4,4,5,5-tetramethyl-1,3,2-dioxaborolan-2-yl)-1H-indazol-3-yl)oxazole

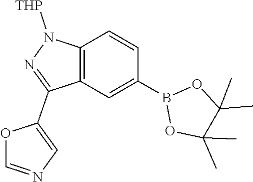

A mixture of 5-(5-bromo-1-(tetrahydro-2H-pyran-2-yl)-1H-indazol-3-yl)oxazole (400 mg, 1.15 mmol), bis(pinacolato)diboron (583.44 mg, 2.30 mmol), Pd(dppf)Cl₂ (84.06 mg, 114.88 umol), KOAc (338.23 mg, 3.45 mmol) in 10 mL of dioxane was degassed and purged with N₂ (3×) at 20° C., and then the mixture was stirred at 120° C. for 1 hr under N₂. The reaction mixture was concentrated and purified by silica gel chromatography using a 0-24% EtOAc/petroleum ether gradient eluent to afford the title compound (400 mg, 88%) as a yellow oil.

### Step 7: 3-(Oxazol-5-yl)-1-(tetrahydro-2H-pyran-2-yl)-1H-indazol-5-ol

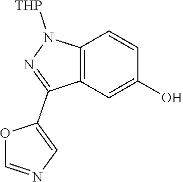

To a solution of 5-(1-(tetrahydro-2H-pyran-2-yl)-5-(4,4,5,5-tetramethyl-1,3,2-dioxaborolan-2-yl)-1H-indazol-3-yl)oxazole (400 mg, 1.01 mmol) in 5 mL of THF and 5 mL of H₂O was added NaBO₃.4H₂O (467.12 mg, 3.04 mmol) at 20° C. The mixture was then stirred at 50° C. for 1 hr. The mixture was extracted with EtOAc and the combined organic layers were dried over Na₂SO₄, filtered and concentrated under reduced pressure to give a residue. The residue was purified by silica gel chromatography using a 0-75% EtOAc/petroleum ether gradient eluent to afford the title compound (220 mg, 76%) as an off-white solid.

### Step 8: 4-Methyl-1-((3-(oxazol-5-yl)-1-(tetrahydro-2H-pyran-2-yl)-1H-indazol-5-yl)oxy)-2,3-dihydro-1H-indene-5-carbonitrile

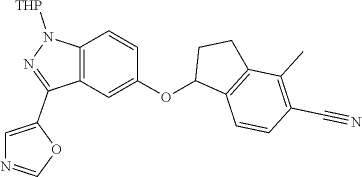

To a solution of 1-hydroxy-4-methyl-2,3-dihydro-1H-indene-5-carbonitrile (40 mg, 230.93 umol), 3-(oxazol-5-yl)-1-(tetrahydro-2H-pyran-2-yl)-1H-indazol-5-ol (65.88 mg, 230.93 umol) in 4 mL of THF was added n-Bu₃P (93.44 mg, 461.87 umol) and ADDP (116.53 mg, 461.87 umol) at 0° C., the mixture was stirred at 45° C. for 1 hr. The mixture was filtered and the filtrate was concentrated and purified by silica gel chromatography using a 0-25% EtOAc/petroleum ether gradient eluent to afford the title compound (50 mg, 49%) as a yellow oil.

### Step 9: 4-Methyl-1-((3-(oxazol-5-yl)-1H-indazol-5-yl)oxy)-2,3-dihydro-1H-indene-5-carbonitrile

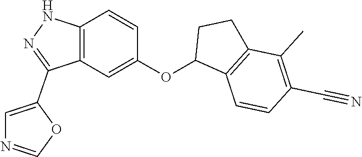 [SMILES: Cc1c(C#N)ccc2c1CCC2Oc1ccc2[nH]nc(-c3cnco3)c2c1]

A solution of 4-methyl-1-((3-(oxazol-5-yl)-1-(tetrahydro-2H-pyran-2-yl)-1H-indazol-5-yl)oxy)-2,3-dihydro-1H-indene-5-carbonitrile (50 mg, 113.51 umol) in 2.5 mL of DCM (2.5 mL) and 0.5 mL of TFA was stirred at 20° C. for 4 hrs. Saturated aqueous NaHCO₃ was added to the mixture at 0° C. to pH=8, then the mixture was extracted with DCM. The combined organic layers were dried over Na₂SO₄, filtered and concentrated under reduced pressure to give a residue. The residue was purified Method Y to afford the title compound (6 mg, 15%) as a white solid.

### Step 10: 4-Methyl-1-((3-(oxazol-5-yl)-1H-indazol-5-yl)oxy)-2,3-dihydro-1H-indene-5-carbonitrile, enantiomer 1 and 2

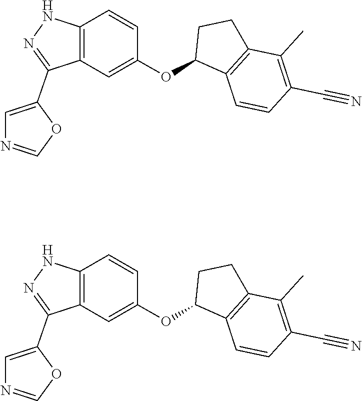 [SMILES: Cc1c(C#N)ccc2c1CC[C@@H]2Oc1ccc2[nH]nc(-c3cnco3)c2c1 ; Cc1c(C#N)ccc2c1CC[C@H]2Oc1ccc2[nH]nc(-c3cnco3)c2c1]

4-Methyl-1-((3-(oxazol-5-yl)-1H-indazol-5-yl)oxy)-2,3-dihydro-1H-indene-5-carbonitrile was subjected to chiral separation using Method Z to afford 4-methyl-1-((3-(oxazol-5-yl)-1H-indazol-5-yl)oxy)-2,3-dihydro-1H-indene-5-carbonitrile, enantiomer 1 (1.83 mg, 28%) as a pale yellow solid. ¹H NMR (400 MHz, CDCl₃) δ 8.47 (s, 1H), 7.77 (s, 1H), 7.65 (d, J=7.70 Hz, 1H), 7.50-7.59 (m, 2H), 7.41 (d, J=7.70 Hz, 1H), 7.08 (br d, J=10.64 Hz, 1H), 6.02-6.10 (m, 1H), 3.02-3.11 (m, 1H), 2.86-2.96 (m, 1H), 2.63-2.69 (m, 1H), 2.46 (s, 3H), 2.09-2.17 (m, 1H). MS-ESI (m/z) calc'd for C₂₁H₁₇N₄O₂ [M+H]⁺: 357.1. Found 357.1. A second fraction was isolated to afford 4-methyl-1-((3-(oxazol-5-yl)-1H-indazol-5-yl)oxy)-2,3-dihydro-1H-indene-5-carbonitrile, enantiomer 2 (2.23 mg, 36%) as a pale yellow solid. ¹H NMR (400 MHz, CDCl₃) δ 13.42 (br s, 1H), 8.51 (s, 1H), 7.84 (s, 1H), 7.66 (d, J=7.70 Hz, 1H), 7.52-7.60 (m, 2H), 7.42 (d, J=7.82 Hz, 1H), 7.15 (dd, J=2.20, 9.05 Hz, 1H), 6.01-6.16 (m, 1H), 3.00-3.12 (m, 1H), 2.85-2.97 (m, 1H), 2.61-2.73 (m, 1H), 2.46 (s, 3H), 2.09-2.17 (m, 1H). MS-ESI (m/z) calc'd for C₂₁H₁₇N₄O₂ [M+H]⁺: 357.1. Found 357.1.

### Example 23: 6-Fluoro-1-((3-(oxazol-5-yl)-1H-indazol-5-yl)oxy)-2,3-dihydro-1H-indene-5-carbonitrile, enantiomer 1 and 2

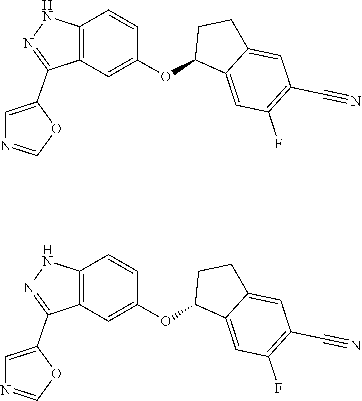 [SMILES: N#Cc1cc2c(cc1F)[C@@H](Oc1ccc3[nH]nc(-c4cnco4)c3c1)CC2 ; N#Cc1cc2c(cc1F)[C@H](Oc1ccc3[nH]nc(-c4cnco4)c3c1)CC2]

Prepared as described for 4-methyl-1-((3-(oxazol-5-yl)-1H-indazol-5-yl)oxy)-2,3-dihydro-1H-indene-5-carbonitrile, enantiomer 1 and 2 using 6-fluoro-1-hydroxy-2,3-dihydro-1H-indene-5-carbonitrile in place of 1-hydroxy-4-methyl-2,3-dihydro-1H-indene-5-carbonitrile to afford 6-fluoro-1-((3-(oxazol-5-yl)-1H-indazol-5-yl)oxy)-2,3-dihydro-1H-indene-5-carbonitrile, enantiomer 1 (4.2 mg, 46%). ¹H NMR (400 MHz, DMSO-d₆) δ 13.42 (s, 1H), 8.52 (s, 1H), 7.92 (d, J=5.87 Hz, 1H), 7.86 (s, 1H), 7.57-7.63 (m, 2H), 7.56 (d, J=2.08 Hz, 1H), 7.19 (dd, J=2.26, 9.11 Hz, 1H), 6.07 (t, J=5.87 Hz, 1H), 3.02-3.14 (m, 1H), 2.87-3.00 (m, 1H), 2.73 (br dd, J=5.99, 12.96 Hz, 1H), 2.08-2.20 (m, 1H). MS-ESI (m/z) calc'd for C₂₀H₁₄FN₄O₂ [M+H]⁺: 361.1. Found 361.1. A second fraction was isolated to afford 6-fluoro-1-((3-(oxazol-5-yl)-1H-indazol-5-yl)oxy)-2,3-dihydro-1H-indene-5-carbonitrile, enantiomer 2 (2.23 mg, 36%) as a pale yellow solid. ¹H NMR (400 MHz, DMSO-d₆) δ 13.42 (s, 1H), 8.52 (s, 1H), 7.92 (d, J=5.87 Hz, 1H), 7.86 (s, 1H), 7.57-7.64 (m, 2H), 7.56 (d, J=2.32 Hz, 1H), 7.18 (dd, J=2.20, 9.05 Hz, 1H), 6.07 (t, J=6.11 Hz, 1H), 3.07 (br dd, J=8.07, 13.33 Hz, 1H), 2.94 (td, J=7.93, 15.80 Hz, 1H), 2.72 (br dd, J=5.14, 11.98 Hz, 1H), 2.14 (dt, J=6.42, 13.48 Hz, 1H). MS-ESI (m/z) calc'd for C₂₀H₁₄FN₄O₂ [M+H]⁺: 361.1. Found 361.1.

### Example 24: 4-Fluoro-1-((3-(oxazol-5-yl)-1H-indazol-5-yl)oxy)-2,3-dihydro-1H-indene-5-carbonitrile, enantiomer 1 and 2

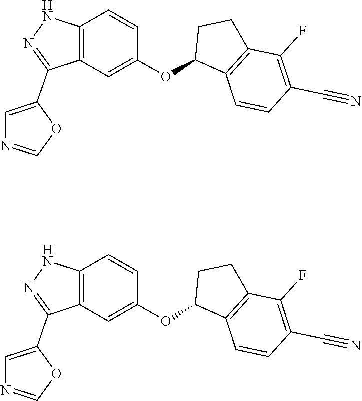 [SMILES: N#Cc1ccc2c(c1F)CC[C@@H]2Oc1ccc2[nH]nc(-c3cnco3)c2c1 ; N#Cc1ccc2c(c1F)CC[C@H]2Oc1ccc2[nH]nc(-c3cnco3)c2c1]

Prepared as described for 4-methyl-1-((3-(oxazol-5-yl)-1H-indazol-5-yl)oxy)-2,3-dihydro-1H-indene-5-carbonitrile, enantiomer 1 and 2 using 4-fluoro-1-hydroxy-2,3-dihydro-1H-indene-5-carbonitrile in place of 1-hydroxy-4-methyl-2,3-dihydro-1H-indene-5-carbonitrile. The first fraction was isolated to afford 4-fluoro-1-((3-(oxazol-5-yl)-1H-indazol-5-yl)oxy)-2,3-dihydro-1H-indene-5-carbonitrile, enantiomer 1 (4.2 mg, 46%). ¹H NMR (400 MHz, DMSO-d₆) δ 13.43 (br s, 1H), 8.51 (s, 1H), 7.86 (s, 1H), 7.81 (t, J=6.84 Hz, 1H), 7.54-7.61 (m, 2H), 7.45 (d, J=7.72 Hz, 1H), 7.13-7.20 (m, 1H), 6.14 (t, J=5.73 Hz, 1H), 3.10-3.21 (m, 1H), 2.94-3.06 (m, 1H), 2.74 (dt, J=7.39, 13.29 Hz, 1H), 2.12-2.23 (m, 1H). MS-ESI (m/z) calc'd for C₂₀H₁₄FN₄O₂ [M+H]⁺: 361.1. Found 361.1. A second fraction was isolated to afford 4-fluoro-1-((3-(oxazol-5-yl)-1H-indazol-5-yl)oxy)-2,3-dihydro-1H-indene-5-carbonitrile, enantiomer 2 (2.23 mg, 36%) as a pale yellow solid. ¹H NMR (400 MHz, DMSO-d₆) δ 13.43 (br s, 1H), 8.52 (s, 1H), 7.86 (s, 1H), 7.78-7.84 (m, 1H), 7.54-7.62 (m, 2H), 7.45 (d, J=7.94 Hz, 1H), 7.17 (dd, J=2.21, 9.04 Hz, 1H), 6.14 (t, J=5.62 Hz, 1H), 3.11-3.21 (m, 1H), 2.96-3.06 (m, 1H), 2.74 (dt, J=7.61, 13.06 Hz, 1H), 2.13-2.24 (m, 1H). MS-ESI (m/z) calc'd for C₂₀H₁₄FN₄O₂ [M+H]⁺: 361.1. Found 361.1.

### Example 25: 6-Fluoro-1-((3-(oxazol-5-yl)-1H-indazol-5-yl)amino)-2,3-dihydro-1H-indene-5-carbonitrile, enantiomer 1 and 2

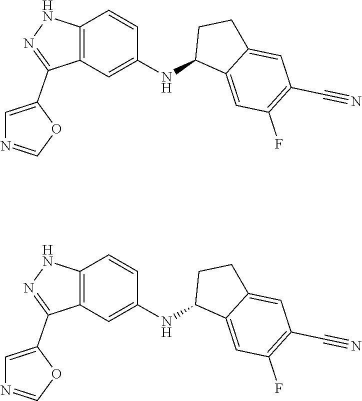 [SMILES: N#Cc1cc2c(cc1F)[C@@H](Nc1ccc3[nH]nc(-c4cnco4)c3c1)CC2 ; N#Cc1cc2c(cc1F)[C@H](Nc1ccc3[nH]nc(-c4cnco4)c3c1)CC2]

### Step 1: N-(5-Bromo-6-fluoro-2,3-dihydro-1H-inden-1-yl)-3-(oxazol-5-yl)-1H-indazol-5-amine

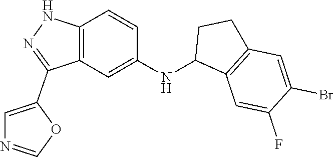 [SMILES: Fc1cc2c(cc1Br)CCC2Nc1ccc2[nH]nc(-c3cnco3)c2c1]

To a solution of 5-bromo-6-fluoro-2,3-dihydro-1H-inden-1-one (100 mg, 436.59 umol), 3-(oxazol-5-yl)-1H-indazol-5-amine (174.81 mg, 873.19 umol) in 2 mL of MeOH and 2 mL of DCE was added AcOH (2.62 mg, 43.66 umol) to pH=5. The mixture was stirred at 20° C. for 3 hrs, then NaBH₃CN (137.18 mg, 2.18 mmol) was added. The reaction mixture was stirred at 20° C. for 12 hrs and then at 40° C. for an additional 12 hrs. The reaction mixture was purified by preparative TLC (SiO₂, Rf=0.30) using EtOAc as eluent to afford the title compound (130 mg, 59%) as a yellow oil.

### Step 2: 6-Fluoro-1-((3-(oxazol-5-yl)-1H-indazol-5-yl)amino)-2,3-dihydro-1H-indene-5-carbonitrile

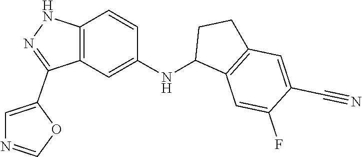 [SMILES: N#Cc1cc2c(cc1F)C(Nc1ccc3[nH]nc(-c4cnco4)c3c1)CC2]

To a solution of N-(5-bromo-6-fluoro-2,3-dihydro-1H-inden-1-yl)-3-(oxazol-5-yl)-1H-indazol-5-amine (13 mg, 31.46 umol), Zn(CN)₂ (11.08 mg, 94.38 umol), and Zn (411.41 ug, 6.29 umol) in 2 mL of DMA in a microwave tube was added dppf (3.49 mg, 6.29 umol) and Pd₂(dba)₃ (5.76 mg, 6.29 umol) under N₂ at 20° C. The sealed tube was irradiated at 100° C. for 2 hrs. The reaction mixture was adjusted to pH=8 by saturated aqueous NaHCO₃. The aqueous phase was extracted with DCM and the combined organic phases were dried over Na₂SO₄, filtered and concentrated. The residue was purified by preparative TLC (SiO₂, Rf=0.50) using EtOAc as eluent to afford the title compound (6 mg, 18%) as a pale yellow solid.

### Step 3: 6-Fluoro-1-((3-(oxazol-5-yl)-1H-indazol-5-yl)amino)-2,3-dihydro-1H-indene-5-carbonitrile, enantiomer 1 and 2

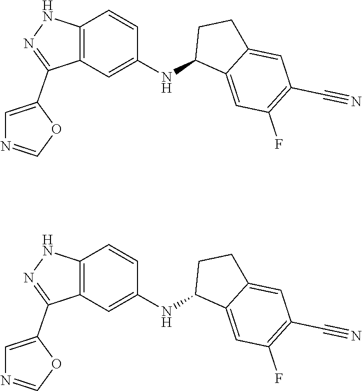 [SMILES: N#Cc1cc2c(cc1F)[C@@H](Nc1ccc3[nH]nc(-c4cnco4)c3c1)CC2 ; N#Cc1cc2c(cc1F)[C@H](Nc1ccc3[nH]nc(-c4cnco4)c3c1)CC2]

6-Fluoro-1-((3-(oxazol-5-yl)-1H-indazol-5-yl)amino)-2,3-dihydro-1H-indene-5-carbonitrile was subjected to chiral separation using Method AA to afford 6-fluoro-1-((3-(oxazol-5-yl)-1H-indazol-5-yl)amino)-2,3-dihydro-1H-indene-5-carbonitrile, enantiomer 1 (1.06 mg, 3%) as a pale yellow solid. ¹H NMR (400 MHz, CD₃OD) δ 8.33 (s, 1H), 7.63 (d, J=6.0 Hz, 1H), 7.59 (s, 1H), 7.42 (d, J=9.0 Hz, 1H), 7.27 (d, J=9.3 Hz, 1H), 7.21 (s, 1H), 7.08 (dd, J=2.0, 8.9 Hz, 1H), 5.24 (t, J=7.7 Hz, 1H), 3.11-2.89 (m, 2H), 2.78-2.69 (m, 1H), 2.00 (qd, J=8.6, 12.6 Hz, 1H). MS-ESI (m/z) calc'd for C₂₀H₁₅FN₅O [M+H]⁺: 360.1. Found 360.1. A second fraction was isolated to afford 6-fluoro-1-((3-(oxazol-5-yl)-1H-indazol-5-yl)amino)-2,3-dihydro-1H-indene-5-carbonitrile, enantiomer 2 (1.53 mg, 4%) as a pale yellow solid. ¹H NMR (400 MHz, CD₃OD) δ 8.32 (s, 1H), 7.63 (d, J=5.9 Hz, 1H), 7.58 (s, 1H), 7.42 (d, J=9.0 Hz, 1H), 7.27 (d, J=9.3 Hz, 1H), 7.20 (s, 1H), 7.08 (dd, J=1.9, 8.9 Hz, 1H), 5.23 (t, J=7.6 Hz, 1H), 3.10-2.88 (m, 2H), 2.79-2.66 (m, 1H), 2.00 (qd, J=8.7, 12.7 Hz, 1H). MS-ESI (m/z) calc'd for C₂₀H₁₅FN₅O [M+H]⁺: 360.1. Found 360.1.

### Example 26: 4-Fluoro-1-((3-(oxazol-5-yl)-1H-indazol-5-yl)amino)-2,3-dihydro-1H-indene-5-carbonitrile, enantiomer 1 and 2

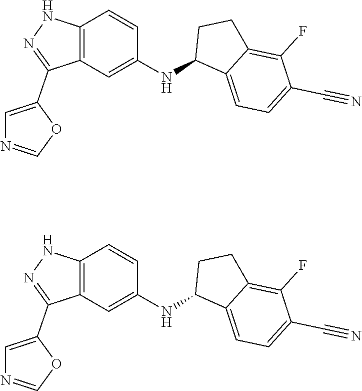 [SMILES: N#Cc1ccc2c(c1F)CC[C@@H]2Nc1ccc2[nH]nc(-c3cnco3)c2c1 ; N#Cc1ccc2c(c1F)CC[C@H]2Nc1ccc2[nH]nc(-c3cnco3)c2c1]

Prepared as described for 6-fluoro-1-((3-(oxazol-5-yl)-1H-indazol-5-yl)amino)-2,3-dihydro-1H-indene-5-carbonitrile enantiomer 1 and 2 using 5-bromo-4-fluoro-2,3-dihydro-1H-inden-1-one in place of 5-bromo-6-fluoro-2,3-dihydro-1H-inden-1-one to afford 4-fluoro-1-((3-(oxazol-5-yl)-1H-indazol-5-yl)oxy)-2,3-dihydro-1H-indene-5-carbonitrile, enantiomer 1 (1.12 mg, 3%). ¹H NMR (400 MHz, DMSO-d₆) δ 8.31 (s, 1H), 7.61-7.56 (m, 1H), 7.56-7.48 (m, 1H), 7.42 (d, J=9.0 Hz, 1H), 7.33 (d, J=7.9 Hz, 1H), 7.22 (s, 1H), 7.07 (dd, J=2.2, 9.0 Hz, 1H), 5.28 (t, J=7.5 Hz, 1H), 3.22-3.09 (m, 1H), 2.98 (td, J=8.3, 16.4 Hz, 1H), 2.82-2.67 (m, 1H), 2.04 (qd, J=8.4, 12.8 Hz, 1H). MS-ESI (m/z) calc'd for C₂₀H₁₅FN₅O [M+H]⁺: 360.1. Found 360.2. A second fraction was isolated to afford 4-fluoro-1-((3-(oxazol-5-yl)-1H-indazol-5-yl)oxy)-2,3-dihydro-1H-indene-5-carbonitrile, enantiomer 2 (1.51 mg, 4%) as a pale yellow solid. ¹H NMR (400 MHz, DMSO-d₆) δ 13.43 (br s, 1H), 8.52 (s, 1H), 7.86 (s, 1H), 7.78-7.84 (m, 1H), 7.54-7.62 (m, 2H), 7.45 (d, J=7.94 Hz, 1H), 7.17 (dd, J=2.21, 9.04 Hz, 1H), 6.14 (t, J=5.62 Hz, 1H), 3.11-3.21 (m, 1H), 2.96-3.06 (m, 1H), 2.74 (dt, J=7.61, 13.06 Hz, 1H), 2.13-2.24 (m, 1H). MS-ESI (m/z) calc'd for C₂₀H₁₅FN₅O [M+H]⁺: 360.1. Found 360.2.

### Example 27: 4-Methyl-1-((3-(oxazol-5-yl)-1H-indazol-5-yl)amino)-2,3-dihydro-1H-indene-5-carbonitrile, enantiomer 1 and 2

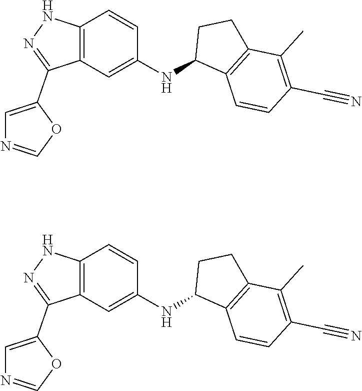 [SMILES: Cc1c(C#N)ccc2c1CC[C@@H]2Nc1ccc2[nH]nc(-c3cnco3)c2c1 ; Cc1c(C#N)ccc2c1CC[C@H]2Nc1ccc2[nH]nc(-c3cnco3)c2c1]

### Step 1: N-(5-Bromo-4-methyl-2,3-dihydro-1H-inden-1-yl)-3-(oxazol-5-yl)-1H-indazol-5-amine

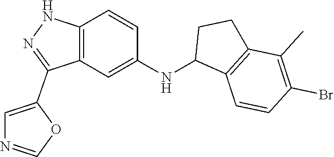 [SMILES: Cc1c(Br)ccc2c1CCC2Nc1ccc2[nH]nc(-c3cnco3)c2c1]

To a solution of 3-(oxazol-5-yl)-1H-indazol-5-amine (177.89 mg, 888.57 umol) and 5-bromo-4-methyl-2,3-dihydro-1H-inden-1-one (100 mg, 444.28 umol) in 10 mL of toluene was added Ti(i-PrO)₄ (631.36 mg, 2.22 mmol) and the reaction mixture was stirred at 130° C. for 16 hrs. After cooling to 20° C., the reaction mixture was concentrated. The residue was dissolved into MeOH (10 mL) and then NaBH₄ (134.47 mg, 3.55 mmol) was added at 0° C. Then the reaction mixture was stirred 20° C. for 4 hrs. After cooling to 0° C., the reaction mixture was poured into saturated aqueous NH₄Cl (30 mL) and the mixture was stirred at 0° C. for 1 hr. The reaction mixture was then poured into water (10 mL) and extracted with EtOAc. The combined organic phases were concentrated. The residue was purified by column chromatography on silica gel using a 0-33% EtOAc/ether gradient eluent to afford the title compound (95 mg, 52%) as a dark brown solid.

### Step 2: 4-Methyl-1-((3-(oxazol-5-yl)-1H-indazol-5-yl)amino)-2,3-dihydro-1H-indene-5-carbonitrile

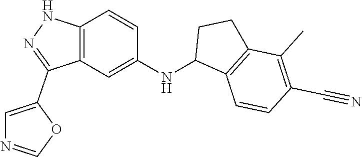 [SMILES: Cc1c(C#N)ccc2c1CCC2Nc1ccc2[nH]nc(-c3cnco3)c2c1]

A mixture of N-(5-bromo-4-methyl-2,3-dihydro-1H-inden-1-yl)-3-(oxazol-5-yl)-1H-indazol-5-amine (50 mg, 122.17 umol), Zn(CN)₂ (43.04 mg, 366.50 umol), Pd₂(dba)₃ (22.37 mg, 24.43 umol), dppf (13.55 mg, 24.43 umol), Zn (23.97 mg, 366.50 umol) in 3 mL of DMA was degassed and purged with N₂ (3×) at 20° C. in a microwave vial. The mixture was stirred under N₂ and irradiated at 120° C. for 2 hrs. After cooling to 20° C., the reaction mixture was adjusted to pH=8 with saturated aqueous NaHCO₃. The reaction mixture was poured into water (5 mL) and extracted with EtOAc. The combined organic phases were concentrated and the residue was purified by preparative TLC (SiO₂, Rf=0.50) using EtOAc as eluent to afford the title compound (9 mgs, 21%).

### Step 3: 4-Methyl-1-((3-(oxazol-5-yl)-1H-indazol-5-yl)amino)-2,3-dihydro-1H-indene-5-carbonitrile, enantiomer 1 and 2

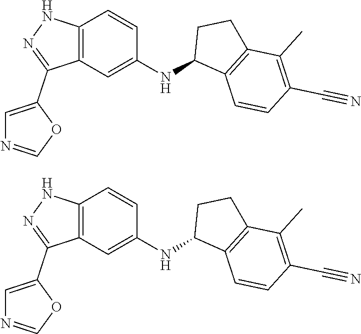

4-Methyl-1-((3-(oxazol-5-yl)-1H-indazol-5-yl)amino)-2,3-dihydro-1H-indene-5-carbonitrile was subjected to chiral separation using Method AB to afford 4-methyl-1-((3-(oxazol-5-yl)-1H-indazol-5-yl)amino)-2,3-dihydro-1H-indene-5-carbonitrile, enantiomer 1 (3.32 mg, 7%) as a pale yellow solid. ¹H NMR (400 MHz, CD₃OD) δ 8.31 (s, 1H), 7.55 (s, 1H), 7.49 (d, J=7.9 Hz, 1H), 7.41 (d, J=8.6 Hz, 1H), 7.34 (d, J=7.7 Hz, 1H), 7.19 (d, J=2.0 Hz, 1H), 7.07 (dd, J=2.2, 9.0 Hz, 1H), 5.21 (t, J=7.4 Hz, 1H), 3.12-3.01 (m, 1H), 2.91 (td, J=8.2, 16.3 Hz, 1H), 2.76-2.64 (m, 1H), 2.49 (s, 3H), 2.06-1.93 (m, 1H). MS-ESI (m/z) calc'd for C₂₁H₁₈N₅O [M+H]⁺: 356.1. Found 356.2. A second fraction was isolated to afford 4-methyl-1-((3-(oxazol-5-yl)-1H-indazol-5-yl)amino)-2,3-dihydro-1H-indene-5-carbonitrile, enantiomer 2 (4.13 mg, 9%) as a pale yellow solid. ¹H NMR (400 MHz, CD₃OD) δ 8.31 (s, 1H), 7.55 (s, 1H), 7.49 (d, J=7.9 Hz, 1H), 7.41 (d, J=8.6 Hz, 1H), 7.34 (d, J=7.7 Hz, 1H), 7.19 (d, J=2.0 Hz, 1H), 7.07 (dd, J=2.2, 9.0 Hz, 1H), 5.21 (t, J=7.4 Hz, 1H), 3.12-3.01 (m, 1H), 2.91 (td, J=8.2, 16.3 Hz, 1H), 2.76-2.65 (m, 1H), 2.49 (s, 3H), 2.05-1.94 (m, 1H). MS-ESI (m/z) calc'd for C₂₁H₁₈N₅O [M+H]⁺: 356.1. Found 356.2.

### Example 28: 6-Methyl-1-((3-(oxazol-5-yl)-1H-indazol-5-yl)amino)-2,3-dihydro-1H-indene-5-carbonitrile, enantiomer 1 and 2

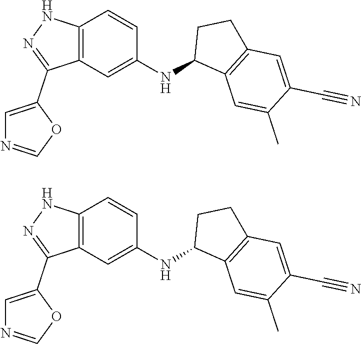 [SMILES: Cc1cc2c(cc1C#N)CC[C@@H]2Nc1ccc2[nH]nc(-c3cnco3)c2c1 ; Cc1cc2c(cc1C#N)CC[C@H]2Nc1ccc2[nH]nc(-c3cnco3)c2c1]

Prepared as described for 4-methyl-1-((3-(oxazol-5-yl)-1H-indazol-5-yl)amino)-2,3-dihydro-1H-indene-5-carbonitrile, enantiomer 1 and 2 using 5-bromo-6-methyl-2,3-dihydro-1H-inden-1-one in place of 5-bromo-4-methyl-2,3-dihydro-1H-inden-1-one to afford 6-methyl-1-((3-(oxazol-5-yl)-1H-indazol-5-yl)amino)-2,3-dihydro-1H-indene-5-carbonitrile, enantiomer 1 (3.94 mg, 23%). ¹H NMR (400 MHz, DMSO-d₆) δ 8.32 (s, 1H), 7.56 (d, J=3.9 Hz, 2H), 7.45-7.35 (m, 2H), 7.19 (s, 1H), 7.11-7.02 (m, 1H), 5.22-5.13 (m, 1H), 3.12-3.00 (m, 1H), 2.99-2.87 (m, 1H), 2.75-2.62 (m, 1H), 2.46 (s, 3H), 2.06-1.88 (m, 1H). MS-ESI (m/z) calc'd for C₂₁H₁₈N₅O [M+H]⁺: 356.1. Found 356.2. A second fraction was isolated to afford 6-methyl-1-((3-(oxazol-5-yl)-1H-indazol-5-yl)amino)-2,3-dihydro-1H-indene-5-carbonitrile, enantiomer 2 (3.94 mg, 23%) as a pale yellow solid. ¹H NMR (400 MHz, DMSO-d₆) δ 8.32 (s, 1H), 7.59-7.53 (m, 2H), 7.43-7.38 (m, 2H), 7.19 (s, 1H), 7.08 (d, J=8.8 Hz, 1H), 5.17 (t, J=7.5 Hz, 1H), 3.03 (br d, J=8.3 Hz, 1H), 2.99-2.87 (m, 1H), 2.69 (br d, J=11.4 Hz, 1H), 2.47 (s, 3H), 2.06-1.89 (m, 1H). MS-ESI (m/z) calc'd for C₂₁H₁₈N₅O [M+H]⁺: 356.1. Found 356.2.

### Example 29: 6-Methyl-1-((3-(oxazol-5-yl)-1H-indazol-5-yl)oxy)-2,3-dihydro-1H-indene-5-carbonitrile, enantiomer 1 and 2

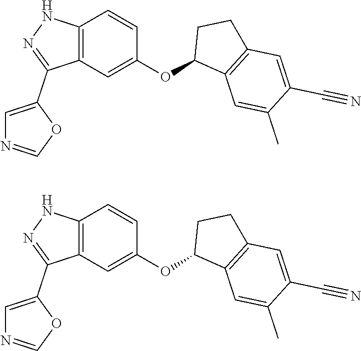 [SMILES: Cc1cc2c(cc1C#N)CC[C@@H]2Oc1ccc2[nH]nc(-c3cnco3)c2c1 ; Cc1cc2c(cc1C#N)CC[C@H]2Oc1ccc2[nH]nc(-c3cnco3)c2c1]

### Step 1: 5-Bromo-6-methyl-2,3-dihydro-1H-inden-1-ol

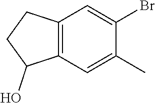

To a solution of 5-bromo-6-methyl-indan-1-one (200 mg, 888.57 umol) in EtOH (2 mL) was added NaBH₄ (50.42 mg, 1.33 mmol) at 20° C. The mixture was stirred at 60° C. for 10 min. After cooling to 20° C., the reaction mixture was concentrated under reduced pressure to remove solvent. The residue was diluted with H₂O and extracted with EtOAc (3×). The combined organic layers were dried over Na₂SO₄, filtered, and concentrated under reduced pressure to afford the title compound (120 mg, 59%) as a white solid.

### Step 2: 5-(5-((5-Bromo-6-methyl-2,3-dihydro-1H-inden-1-yl)oxy)-1-(tetrahydro-2H-pyran-2-yl)-1H-indazol-3-yl)oxazole

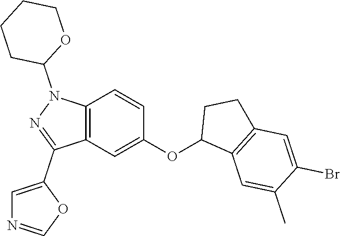 [SMILES: Cc1cc2c(cc1Br)CCC2Oc1ccc2c(c1)c(-c1cnco1)nn2C1CCCCO1]

To a stirred solution of 3-(oxazol-5-yl)-1-(tetrahydro-2H-pyran-2-yl)-1H-indazol-5-ol (100 mg, 350.51 umol) and 5-bromo-6-methyl-2,3-dihydro-1H-inden-1-ol (79.60 mg, 350.51 umol) in toluene (4 mL) was added tributylphosphine (141.83 mg, 701.02 umol) and 1,1′-(azodicarbonyl)dipiperidine (176.88 mg, 701.02 umol) at 0° C. under N₂. The reaction mixture was then stirred at 60° C. for 12 hrs under N₂. The reaction mixture was diluted with H₂O and the aqueous phase was extracted with EtOAc (3×). The combined organic phases were concentrated and purified by preparative-TLC (SiO₂, petroleum ether/EtOAc=1:1, Rf=0.43) to afford the title compound (80 mg, 46%) as a light yellow oil. MS-ESI (m/z) calc'd for C₂₅H₂₅BrN₃O₃ [M+H]⁺: 494.1/496.1. Found 494.1/496.1.

### Step 3: 6-Methyl-1-((3-(oxazol-5-yl)-1-(tetrahydro-2H-pyran-2-yl)-1H-indazol-5-yl)oxy)-2,3-dihydro-1H-indene-5-carbonitrile

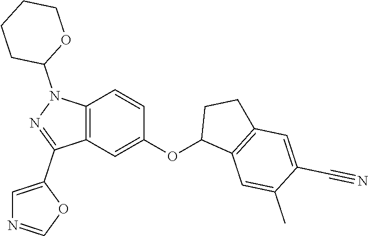 [SMILES: Cc1cc2c(cc1C#N)CCC2Oc1ccc2c(c1)c(-c1cnco1)nn2C1CCCCO1]

A mixture of 5-(5-((5-bromo-6-methyl-2,3-dihydro-1H-inden-1-yl)oxy)-1-(tetrahydro-2H-pyran-2-yl)-1H-indazol-3-yl)oxazole (80 mg, 161.82 umol), Zn(CN)₂ (28.50 mg, 242.73 umol), 1,1-bis(diphenylphosphino)ferrocene (8.97 mg, 16.18 umol), Pd₂dba₃ (9.30 mg, 16.18 umol) and Zn (1.06 mg, 16.18 umol) in DMA (2 mL) was added to a microwave vial. The vial was degassed and purged with N₂ (3×) at 20° C., and then the mixture was stirred at 120° C. for 2 hrs under an N₂ atmosphere. After cooling to 20° C., the reaction mixture was adjusted to pH 8 with a saturated aqueous NaHCO₃ solution. The reaction mixture was poured into H₂O and extracted with EtOAc (3×). The combined organic phases were concentrated and purified by preparative-TLC (SiO₂, petroleum ether/EtOAc=1:1, Rf=0.50) to afford the title compound (30 mg, 42%) as a yellow oil. MS-ESI (m/z) calc'd for C₂₆H₂₅N₄O₃ [M+H]⁺: 441.2. Found 441.2.

### Step 4: 6-Methyl-1-((3-(oxazol-5-yl)-1H-indazol-5-yl)oxy)-2,3-dihydro-1H-indene-5-carbonitrile, enantiomer 1 and 2

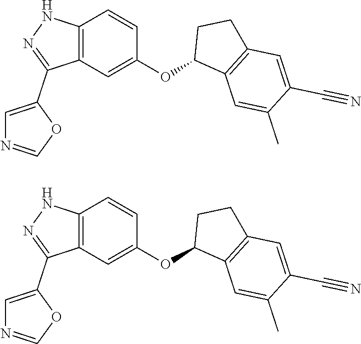 [SMILES: Cc1cc2c(cc1C#N)CC[C@H]2Oc1ccc2[nH]nc(-c3cnco3)c2c1 ; Cc1cc2c(cc1C#N)CC[C@@H]2Oc1ccc2[nH]nc(-c3cnco3)c2c1]

To a solution of 6-methyl-1-((3-(oxazol-5-yl)-1-(tetrahydro-2H-pyran-2-yl)-1H-indazol-5-yl)oxy)-2,3-dihydro-1H-indene-5-carbonitrile (30 mg, 68.11 umol) in DCM (2 mL) was added TFA (0.5 mL). The mixture was stirred at 20° C. for 1 hr. The reaction mixture was adjusted to pH 8 with a saturated aqueous NaHCO₃ solution and extracted with EtOAc (3×). The combined organic phases were concentrated in vacuum to give a residue. The residue was purified by preparative-HPLC using Method AC to afford 6-methyl-1-((3-(oxazol-5-yl)-1H-indazol-5-yl)oxy)-2,3-dihydro-1H-indene-5-carbonitrile (9 mg, 37%) as a pale yellow solid. Then the racemate was further separated by SFC method using Method AD to afford 6-methyl-1-((3-(oxazol-5-yl)-1H-indazol-5-yl)oxy)-2,3-dihydro-1H-indene-5-carbonitrile, enantiomer 1 (1.36 mg, 6%) as a white solid. ¹H NMR (400 MHz, MeOD) δ 8.37 (s, 1H), 7.67 (s, 1H), 7.62 (s, 1H), 7.59 (d, J=2.1 Hz, 1H), 7.54 (d, J=9.0 Hz, 1H), 7.45 (s, 1H), 7.20 (dd, J=2.3, 9.0 Hz, 1H), 5.99-5.92 (m, 1H), 3.19-3.06 (m, 1H), 3.03-2.91 (m, 1H), 2.78-2.64 (m, 1H), 2.51 (s, 3H), 2.23 (dddd, J=5.0, 6.3, 8.6, 13.6 Hz, 1H), MS-ESI (m/z) calc'd for C₂₁H₁₇N₄O₂ [M+H]⁺: 357.1. Found 357.2. A later eluting fraction was also isolated to afford 6-methyl-1-((3-(oxazol-5-yl)-1H-indazol-5-yl)oxy)-2,3-dihydro-1H-indene-5-carbonitrile, enantiomer 2 (1.13 mg, 5%) as a white solid. ¹H NMR (400 MHz, MeOD) δ 8.39 (s, 1H), 7.69 (s, 1H), 7.64 (s, 1H), 7.61 (d, J=2.2 Hz, 1H), 7.56 (d, J=9.0 Hz, 1H), 7.47 (s, 1H), 7.22 (dd, J=2.3, 9.0 Hz, 1H), 6.01-5.95 (m, 1H), 3.20-3.10 (m, 1H), 3.06-2.94 (m, 1H), 2.74 (dddd, J=5.1, 6.8, 8.4, 13.5 Hz, 1H), 2.53 (s, 3H), 2.31-2.18 (m, 1H). MS-ESI (m/z) calc'd for C₂₁H₁₇N₄O₂ [M+H]⁺: 357.1. Found 357.2.

### Example 30: 1-((3-(Oxazol-5-yl)-1H-pyrazolo[4,3-b]pyridin-5-yl)amino)-2,3-dihydro-1H-indene-5-carbonitrile, enantiomer 1 and 2

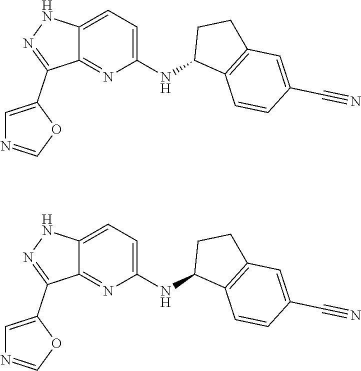 [SMILES: N#Cc1ccc2c(c1)CC[C@H]2Nc1ccc2[nH]nc(-c3cnco3)c2n1 ; N#Cc1ccc2c(c1)CC[C@@H]2Nc1ccc2[nH]nc(-c3cnco3)c2n1]

### Step 1: 5-Chloro-3-iodo-1H-pyrazolo[4,3-b]pyridine

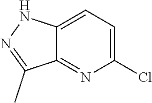

To a solution of 5-chloro-1H-pyrazolo[4,3-b]pyridine (2 g, 13.02 mmol) in DMF (15 mL) was added I₂ (6.61 g, 26.05 mmol) followed by KOH (3.65 g, 65.12 mmol) portion-wise at 0° C. The mixture was stirred at 20° C. for 12 hrs and stirred at 60° C. for another 5 hrs. Additional I₂ (6.61 g, 26.05 mmol) and KOH (3.65 g, 65.12 mmol) was then added to the mixture and stirring was continued at 20° C. for another 12 hrs. The reaction mixture was filtered and the solid was washed with EtOAc (2×). The filtrate was poured into 2N NaOH (200 mL) and extracted with EtOAc (3×); the combined organic layers were dried over Na₂SO₄ and evaporated to dryness to afford material that was further purified by flash silica gel chromatography (ISCO; 40 g SepaFlash column) using a 0-20% EtOAc/petroleum ether gradient eluent to afford the title compound (3.1 g, 85%) as a brown solid. MS-ESI (m/z) calc'd for C₆H₄ClIN₃ [M+H]⁺: 279.9/281.9. Found 279.9/281.9.

### Step 2: 5-Chloro-3-iodo-1-((2-(trimethylsilyl)ethoxy)methyl)-1H-pyrazolo[4,3-b]pyridine

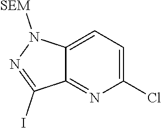 [SMILES: C[Si](C)(C)CCOCn1nc(I)c2nc(Cl)ccc21]

To a solution of 5-chloro-3-iodo-1H-pyrazolo[4,3-b]pyridine (3.1 g, 11.09 mmol) and SEM-Cl (2.77 g, 16.64 mmol) in THF (100 mL) was added N-cyclohexyl-N-methyl-cyclohexanamine (4.33 g, 22.19 mmol). The mixture was stirred at 20° C. for 12 hrs. The solvent was evaporated and the residue was purified by flash silica gel chromatography (ISCO; 40 g SepaFlash column) using a 0-10% EtOAc/petroleum ether gradient eluent to afford the title compound (4.33 g, 95%) as a light yellow oil. MS-ESI (m/z) calc'd for C₁₂H₁₈ClIN₃OSi [M+H]⁺: 410.0/412.0. Found 410.0/412.0.

### Step 3: 5-Chloro-1-((2-(trimethylsilyl)ethoxy)methyl)-3-vinyl-1H-pyrazolo[4,3-b]pyridine

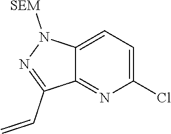 [SMILES: C=Cc1nn(COCC[Si](C)(C)C)c2ccc(Cl)nc12]

A mixture of 5-chloro-3-iodo-1-((2-(trimethylsilyl)ethoxy)methyl)-1H-pyrazolo[4,3-b]pyridine (3.8 g, 9.27 mmol), potassium vinyltrifluoroborate (1.33 g, 9.92 mmol), K₂CO₃ (2.56 g, 18.55 mmol), Pd(dppf)Cl₂ (678.62 mg, 927.45 umol) in THF (70 mL) and H₂O (14 mL) was degassed and purged with N₂ (3×) at 20° C. and then the mixture was stirred at 70° C. for 12 hrs under an N₂ atmosphere. The reaction mixture was evaporated to give a residue that was diluted with H₂O and extracted with EtOAc (3×), the combined organic layers were dried over Na₂SO₄ and evaporated to dryness to afford material that was further purified by flash silica gel chromatography (ISCO; 12 g SepaFlash column) using a 0-5% EtOAc/petroleum ether gradient eluent to afford the title compound (4.33 g, 95%) as a light yellow oil. MS-ESI (m/z) calc'd for C₁₄H₂₁ClN₃OSi [M+H]⁺: 310.1/312.1. Found 310.1/312.1.

### Step 4: 5-Chloro-1-((2-(trimethylsilyl)ethoxy)methyl)-1H-pyrazolo[4,3-b]pyridine-3-carbaldehyde

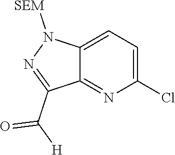 [SMILES: C[Si](C)(C)CCOCn1nc(C=O)c2nc(Cl)ccc21]

To a solution of 5-chloro-1-((2-(trimethylsilyl)ethoxy)methyl)-3-vinyl-1H-pyrazolo[4,3-b]pyridine (2.1 g, 6.78 mmol) in DCM (100 mL) was bubbled ozone (15 psi) for 10 minutes at −60° C. after which triphenylphosphine (8.89 g, 33.89 mmol) was added to the mixture. The mixture was stirred at −60° C. for another 20 minutes. The reaction mixture was evaporated to give a residue and the residue was purified by flash silica gel chromatography (ISCO; 40 g SepaFlash column) using a 0-8% EtOAc/petroleum ether gradient eluent to afford the title compound (2.02 g, 95%) as a light yellow oil. ¹H NMR (400 MHz, CDCl₃) δ 10.45 (s, 1H), 8.05 (d, J=9 Hz, 1H), 7.50 (d, J=9 Hz, 1H), 5.88 (s, 2H), 3.59-3.65 (m, 2H), 0.92-0.97 (m, 2H), −0.01-0.01 (m, 9H). MS-ESI (m/z) calc'd for C₁₃H₁₉ClN₃O₂Si [M+H]⁺: 312.1/314.1. Found 312.1/314.1.

### Step 5: 5-(5-Chloro-1-((2-(trimethylsilyl)ethoxy)methyl)-1H-pyrazolo[4,3-b]pyridin-3-yl)oxazole

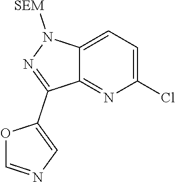 [SMILES: C[Si](C)(C)CCOCn1nc(-c2cnco2)c2nc(Cl)ccc21]

To a solution of 5-chloro-1-(2-trimethylsilylethoxymethyl)pyrazolo[4,3-b]pyridine-3-carbaldehyde (800 mg, 2.57 mmol) in MeOH (25 mL) was added K₂CO₃ (709.12 mg, 5.13 mmol) and TosMIC (751.30 mg, 3.85 mmol) at 20° C. and the mixture was stirred at 60° C. for 0.5 hr. The reaction mixture was evaporated to give a residue and the residue was purified by flash silica gel chromatography (ISCO; 12 g SepaFlash column) using a 0-18% EtOAc/petroleum ether gradient eluent to afford the title compound (750 mg, 83%) as a light yellow solid. MS-ESI (m/z) calc'd for C₁₅H₂₀ClN₄O₂Si [M+H]⁺: 351.1/353.1. Found 351.0/353.0.

### Step 6: 1-((3-(Oxazol-5-yl)-1-((2-(trimethylsilyl)ethoxy)methyl)-1H-pyrazolo[4,3-b]pyridin-5-yl)amino)-2,3-dihydro-1H-indene-5-carbonitrile

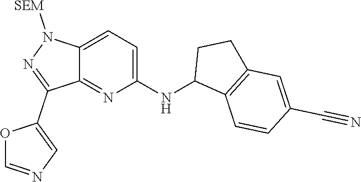 [SMILES: C[Si](C)(C)CCOCn1nc(-c2cnco2)c2nc(NC3CCc4cc(C#N)ccc43)ccc21]

A mixture of 5-(5-chloro-1-((2-(trimethylsilyl)ethoxy)methyl)-1H-pyrazolo[4,3-b]pyridin-3-yl)oxazole (250 mg, 712.51 umol), 1-aminoindane-5-carbonitrile (135.26 mg, 855.01 umol), t-BuONa (2 M, 712.51 uL), tBuXPhos Pd G3 (56.60 mg, 71.25 umol) in THF (8 mL) was degassed and purged with N₂ (3×) at 20° C., and then the mixture was stirred at 60° C. for 12 hrs under an N₂ atmosphere. The reaction mixture was evaporated to give a residue and the residue was purified by flash silica gel chromatography (ISCO; 4 g SepaFlash column) using a 0-40% EtOAc/petroleum ether gradient eluent to afford the title compound (40 mg, 12%) as a yellow oil. MS-ESI (m/z) calc'd for C₂₅H₂₉N₆O₂Si [M+H]⁺: 473.2. Found 473.2.

### Step 7: 1-((1-(Hydroxymethyl)-3-(oxazol-5-yl)-1H-pyrazolo[4,3-b]pyridin-5-yl)amino)-2,3-dihydro-1H-indene-5-carbonitrile

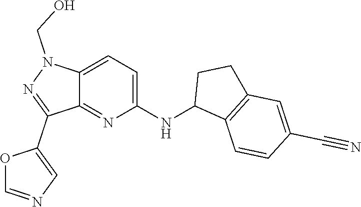 [SMILES: N#Cc1ccc2c(c1)CCC2Nc1ccc2c(n1)c(-c1cnco1)nn2CO]

To a solution of 1-((3-(oxazol-5-yl)-1-((2-(trimethylsilyl)ethoxy)methyl)-1H-pyrazolo[4,3-b]pyridin-5-yl)amino)-2,3-dihydro-1H-indene-5-carbonitrile (35 mg, 74.06 umol) in DCM (1 mL) was added TFA (84.44 mg, 740.56 umol). The mixture was stirred at 20° C. for 4 hrs. The reaction mixture was evaporated to afford the title compound (35 mg, 80%) as a yellow gum. MS-ESI (m/z) calc'd for C₂₀H₁₇N₆O₂ [M+H]⁺: 373.1. Found 373.1.

### Step 8: 1-((3-(Oxazol-5-yl)-1H-pyrazolo[4,3-b]pyridin-5-yl)amino)-2,3-dihydro-1H-indene-5-carbonitrile

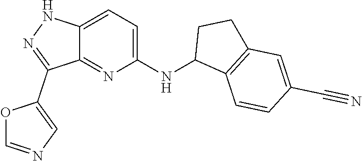 [SMILES: N#Cc1ccc2c(c1)CCC2Nc1ccc2[nH]nc(-c3cnco3)c2n1]

To a solution of 1-[[1-(hydroxymethyl)-3-oxazol-5-yl-pyrazolo[4,3-b]pyridin-5-yl]amino]indane-5-carbonitrile (35 mg, 71.96 umol) in 1,4-dioxane (1.5 mL) was added NH₄OH (80.70 mg, 575.66 umol) and the mixture was stirred at 20° C. for 4 hrs. The reaction mixture was evaporated to give a residue. The residue was purified by preparative-HPLC using Method AE to afford the title compound (9 mg, 36%) as a white solid. MS-ESI (m/z) calc'd for C₁₉H₁₅N₆O [M+H]⁺: 343.1. Found 343.1.

### Step 9: 1-((3-(Oxazol-5-yl)-1H-pyrazolo[4,3-b]pyridin-5-yl)amino)-2,3-dihydro-1H-indene-5-carbonitrile, enantiomer 1 and 2

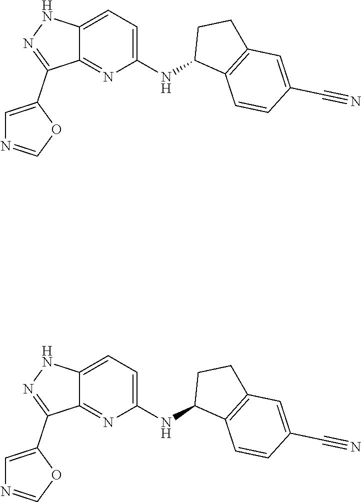 [SMILES: N#Cc1ccc2c(c1)CC[C@H]2Nc1ccc2[nH]nc(-c3cnco3)c2n1 ; N#Cc1ccc2c(c1)CC[C@@H]2Nc1ccc2[nH]nc(-c3cnco3)c2n1]

1-((3-(Oxazol-5-yl)-1H-pyrazolo[4,3-b]pyridin-5-yl)amino)-2,3-dihydro-1H-indene-5-carbonitrile was subjected to chiral separation using Method AF to afford 1-((3-(oxazol-5-yl)-1H-pyrazolo[4,3-b]pyridin-5-yl)amino)-2,3-dihydro-1H-indene-5-carbonitrile, enantiomer 1 (2.41 mg, 27%). ¹H NMR (400 MHz, DMSO-d₆) δ 8.43 (s, 1H), 7.73-7.79 (m, 2H), 7.69 (s, 1H), 7.61 (d, J=7 Hz, 1H), 7.49-7.53 (m, 1H), 7.32 (d, J=8 Hz, 1H), 6.75 (d, J=9 Hz, 1H), 5.72-5.79 (m, 1H), 2.92-3.10 (m, 2H), 2.58-2.65 (m, 1H), 1.97 (dq, J=12, 9 Hz, 1H). MS-ESI (m/z) calc'd for C₁₉H₁₅N₆O [M+H]⁺: 343.1. Found 343.1. A later eluting fraction was also isolated to afford 1-((3-(oxazol-5-yl)-1H-pyrazolo[4,3-b]pyridin-5-yl)amino)-2,3-dihydro-1H-indene-5-carbonitrile, enantiomer 2 (2.32 mg, 26%). ¹H NMR (400 MHz, DMSO-d₆) δ 13.18 (br s, 1H), 8.44 (s, 1H), 7.72-7.78 (m, 2H), 7.70 (s, 1H), 7.61 (d, J=8 Hz, 1H), 7.51 (d, J=8 Hz, 1H), 7.33 (d, J=8 Hz, 1H), 6.76 (d, J=9 Hz, 1H), 5.75 (q, J=8 Hz, 1H), 2.92-3.10 (m, 2H), 2.58-2.65 (m, 1H), 1.97 (dq, J=12, 9 Hz, 1H). MS-ESI (m/z) calc'd for C₁₉H₁₅N₆O [M+H]⁺: 343.1. Found 343.1.

### Example 31: 1-((3-(Oxazol-5-yl)-1H-pyrazolo[4,3-b]pyridin-5-yl)oxy)-2,3-dihydro-1H-indene-5-carbonitrile, enantiomer 1 and 2

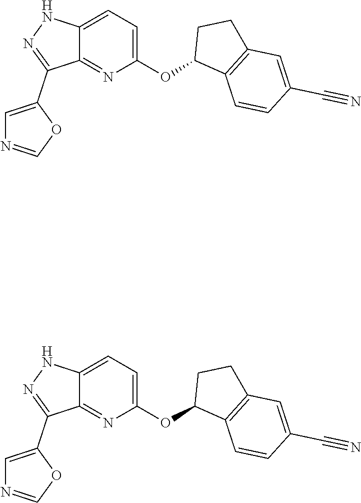 [SMILES: N#Cc1ccc2c(c1)CC[C@H]2Oc1ccc2[nH]nc(-c3cnco3)c2n1 ; N#Cc1ccc2c(c1)CC[C@@H]2Oc1ccc2[nH]nc(-c3cnco3)c2n1]

### Step 1: 5-(5-Chloro-1-((2-(trimethylsilyl)ethoxy)methyl)-1H-pyrazolo[4,3-b]pyridin-3-yl)oxazole

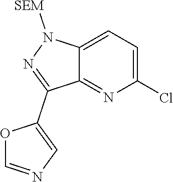 [SMILES: C[Si](C)(C)CCOCn1nc(-c2cnco2)c2nc(Cl)ccc21]

To a solution of 5-chloro-1-(2-trimethylsilylethoxymethyl)pyrazolo[4,3-b]pyridine-3-carbaldehyde (200 mg, 641.36 umol) in MeOH (6 mL) was added K₂CO₃ (177.28 mg, 1.28 mmol) and TosMIC (187.83 mg, 962.03 umol) at 20° C. The mixture was stirred at 60° C. for 0.5 hr. The reaction mixture was evaporated to give a residue and the residue was purified by preparative-TLC (petroleum ether/EtOAc=2:1, Rf=0.53) to afford the title compound (86 mg, 38%) as a white solid. MS-ESI (m/z) calc'd for C₁₅H₂₀ClN₄O₂Si [M+H]⁺: 351.1/353.1. Found 351.1/353.1.

### Step 2: 3-(Oxazol-5-yl)-1-((2-(trimethylsilyl)ethoxy)methyl)-1H-pyrazolo[4,3-b]pyridin-5-ol

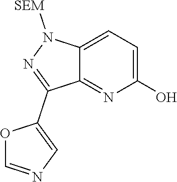 [SMILES: C[Si](C)(C)CCOCn1nc(-c2cnco2)c2nc(O)ccc21]

A mixture of 5-(5-chloro-1-((2-(trimethylsilyl)ethoxy)methyl)-1H-pyrazolo[4,3-b]pyridin-3-yl)oxazole (90 mg, 256.50 umol), KOH (86.35 mg, 1.54 mmol), Pd₂dba₃ (11.74 mg, 12.83 umol), t-BuXphos (10.89 mg, 25.65 umol) in 1,4-dioxane (3 mL) and H₂O (3 mL) was degassed and purged with N₂ (3×) at 20° C., then the mixture was stirred at 60° C. for 12 hrs under an N₂ atmosphere. The reaction mixture was diluted with H₂O, acidified with AcOH to pH=5, and extracted with EtOAc (3×). The combined organic layers were dried over Na₂SO₄ and evaporated to give a residue. The residue was purified by preparative-TLC (100% petroleum ether, Rf=0.23) to afford the title compound (141 mg, 83%) as a gray solid. ¹H NMR (400 MHz, CDCl₃) δ 11.34 (br s, 1H), 8.07 (s, 1H), 7.95 (s, 1H), 7.84 (d, J=9.66 Hz, 1H), 6.77 (d, J=9.66 Hz, 1H), 5.69 (s, 2H), 3.59-3.66 (m, 2H), 0.91-0.98 (m, 2H), −0.04-0.03 (m, 9H). MS-ESI (m/z) calc'd for C₁₅H₂₁N₄O₃Si [M+H]⁺: 333.1. Found 333.1.

### Step 3: 1-((3-(Oxazol-5-yl)-1-((2-(trimethylsilyl)ethoxy)methyl)-1H-pyrazolo[4,3-b]pyridin-5-yl)oxy)-2,3-dihydro-1H-indene-5-carbonitrile

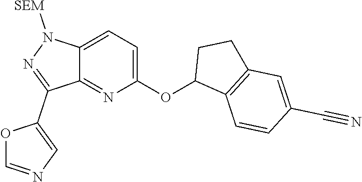 [SMILES: C[Si](C)(C)CCOCn1nc(-c2cnco2)c2nc(OC3CCc4cc(C#N)ccc43)ccc21]

To a solution of 3-oxazol-5-yl-1-(2-trimethylsilylethoxymethyl)pyrazolo[4,3-b]pyridin-5-ol (70 mg, 210.57 umol) and 1-hydroxyindane-5-carbonitrile (67.04 mg, 421.14 umol) in THF (5 mL) was added 1,1′-(azodicarbonyl)dipiperidine (106.26 mg, 421.14 umol) and tributylphosphine (85.21 mg, 421.14 umol) at 0° C. The mixture was stirred at 40° C. for 1 hr. The reaction mixture was cooled to 20° C. and filtered, the filtrate was evaporated to give a residue and the residue was purified by preparative-TLC (petroleum ether/EtOAc=2:1, Rf=0.45) to afford the title compound (90 mg, 99%) as yellow gum. MS-ESI (m/z) calc'd for C₂₅H₂₈N₅O₃Si [M+H]⁺: 474.2. Found 474.1.

### Step 4: 1-((3-(Oxazol-5-yl)-1H-pyrazolo[4,3-b]pyridin-5-yl)oxy)-2,3-dihydro-1H-indene-5-carbonitrile

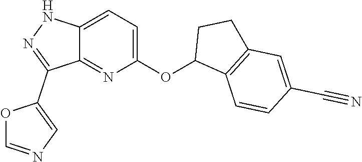 [SMILES: N#Cc1ccc2c(c1)CCC2Oc1ccc2[nH]nc(-c3cnco3)c2n1]

To a solution of 1-[3-oxazol-5-yl-1-(2-trimethylsilylethoxymethyl)pyrazolo[4,3-b]pyridin-5-yl]oxyindane-5-carbonitrile (90 mg, 163.43 umol) in DMF (3 mL) was added TBAF (2.45 mL, 2.45 mmol) and ethane-1,2-diamine (147.33 mg, 2.45 mmol) at 20° C. The reaction mixture was stirred at 90° C. for 3 hrs. The reaction mixture was evaporated to give a residue and the residue was purified by preparative-TLC (100% petroleum ether, Rf=0.23) and further purified by preparative-HPLC using Method AG to afford the title compound (33 mg, 59%) as a white solid. MS-ESI (m/z) calc'd for C₁₉H₁₄N₅O₂ [M+H]⁺: 344.1. Found 344.1.

### Step 5: 1-((3-(Oxazol-5-yl)-1H-pyrazolo[4,3-b]pyridin-5-yl)oxy)-2,3-dihydro-1H-indene-5-carbonitrile, enantiomer 1 and 2

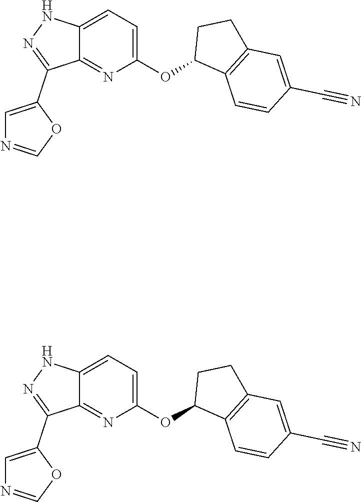 [SMILES: N#Cc1ccc2c(c1)CC[C@H]2Oc1ccc2[nH]nc(-c3cnco3)c2n1 ; N#Cc1ccc2c(c1)CC[C@@H]2Oc1ccc2[nH]nc(-c3cnco3)c2n1]

1-((3-(Oxazol-5-yl)-1H-pyrazolo[4,3-b]pyridin-5-yl)oxy)-2,3-dihydro-1H-indene-5-carbonitrile was subjected to chiral separation using Method AH to afford 1-((3-(oxazol-5-yl)-1H-pyrazolo[4,3-b]pyridin-5-yl)oxy)-2,3-dihydro-1H-indene-5-carbonitrile, enantiomer 1 (12.21 mg, 37%) as a white solid. ¹H NMR (400 MHz, DMSO-d₆) δ 8.51 (br s, 1H), 8.03 (br d, J=9 Hz, 1H), 7.82-7.90 (m, 2H), 7.64-7.72 (m, 2H), 6.80-6.97 (m, 1H), 6.66-6.80 (m, 1H), 2.96-3.22 (m, 2H), 2.77 (td, J=13, 8 Hz, 1H), 2.14-2.24 (m, 1H). MS-ESI (m/z) calc'd for C₁₉H₁₄N₅O₂ [M+H]⁺: 344.1. Found 344.0. A later eluting fraction was also isolated to afford 1-((3-(oxazol-5-yl)-1H-pyrazolo[4,3-b]pyridin-5-yl)oxy)-2,3-dihydro-1H-indene-5-carbonitrile, enantiomer 2 (14.39 mg, 43%) as a white solid. ¹H NMR (400 MHz, DMSO-d₆) δ 13.56 (br s, 1H), 8.52 (s, 1H), 8.04 (d, J=9 Hz, 1H), 7.90 (s, 1H), 7.85 (s, 1H), 7.65-7.71 (m, 2H), 6.93 (d, J=9 Hz, 1H), 6.75 (dd, J=7, 5 Hz, 1H), 2.99-3.19 (m, 2H), 2.73-2.82 (m, 1H), 2.15-2.24 (m, 1H). MS-ESI (m/z) calc'd for C₁₉H₁₄N₅O₂ [M+H]⁺: 344.1. Found 344.0.

### Example 32: 5-((3-(Oxazol-5-yl)-1H-pyrazolo[3,4-c]pyridin-5-yl)oxy)-5,6,7,8-tetrahydronaphthalene-2-carbonitrile, enantiomer 1 and 2

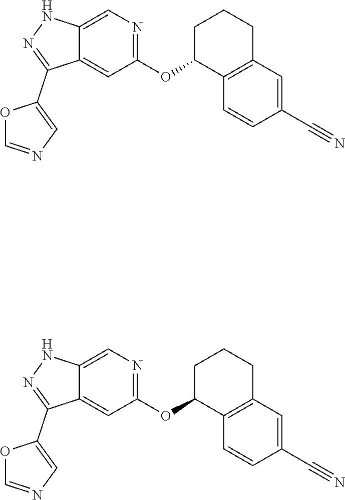 [SMILES: N#Cc1ccc2c(c1)CCC[C@H]2Oc1cc2c(-c3cnco3)n[nH]c2cn1 ; N#Cc1ccc2c(c1)CCC[C@@H]2Oc1cc2c(-c3cnco3)n[nH]c2cn1]

### Step 1: 5-Hydroxy-5,6,7,8-tetrahydronaphthalene-2-carbonitrile

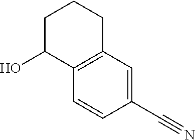 [SMILES: N#Cc1ccc2c(c1)CCCC2O]

To a mixture of 1-oxotetralin-6-carbonitrile (300 mg, 1.75 mmol) in MeOH (4 mL) was added NaBH₄ (79.56 mg, 2.10 mmol) at 0° C., the mixture was degassed and purged with N₂ (3×), and the mixture was stirred at 0° C. for 0.5 hr under an N₂ atmosphere. Then the mixture was stirred at 20° C. for 2 hrs under an N₂ atmosphere. The mixture was concentrated under vacuum, diluted with H₂O, and extracted with EtOAc (3×). The combined organic phases were dried with anhydrous Na₂SO₄ and concentrated to give a residue. The residue was purified by flash silica gel chromatography (ISCO; 4 g SepaFlash column) using a 0-12% EtOAc/petroleum ether gradient eluent to afford the title compound (223 mg, 73%) as a yellow solid. ¹H NMR (400 MHz, CDCl₃) δ 7.58 (d, J=7.94 Hz, 1H), 7.45-7.50 (m, 1H), 7.40 (s, 1H), 4.79 (t, J=5.62 Hz, 1H), 2.70-2.90 (m, 2H), 1.94-2.13 (m, 2H), 1.76-1.93 (m, 3H).

### Step 2: 5-Bromo-1H-pyrazolo[3,4-c]pyridine-3-carbaldehyde

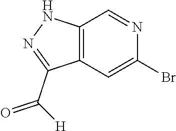 [SMILES: O=Cc1n[nH]c2cnc(Br)cc12]

To a solution of NaNO₂ (1.54 g, 22.33 mmol) in H₂O (5 mL) was added HCl (2 M, 9.77 mL) at 0° C. dropwise slowly, then the mixture was stirred at 0° C. for 10 min before adding DMF (10 mL). A solution of 5-bromo-1H-pyrrolo[2,3-c]pyridine (550 mg, 2.79 mmol) in DMF (10 mL) was then added at 0° C. and the mixture was heated to 80° C. and stirred for 12 hrs. The reaction mixture was diluted with H₂O and extracted with EtOAc (3×). The combined organic phases were dried with anhydrous Na₂SO₄ and evaporated to give a residue. The residue was purified by flash silica gel chromatography (ISCO; 20 g SepaFlash column) using a 0-22% EtOAc/petroleum ether gradient eluent to afford the title compound (615 mg, 49%) as a white solid. MS-ESI (m/z) calc'd for C₇H₅BrN₃O [M+H]⁺: 226.0/228.0. Found 225.9/227.9.

### Step 3: 5-Bromo-1-((2-(trimethylsilyl)ethoxy)methyl)-1H-pyrazolo[3,4-c]pyridine-3-carbaldehyde

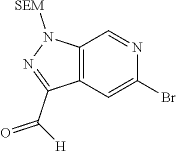 [SMILES: C[Si](C)(C)CCOCn1nc(C=O)c2cc(Br)ncc21]

To a solution of 5-bromo-1H-pyrazolo[3,4-c]pyridine-3-carbaldehyde (615 mg, 2.72 mmol) in THF (20 mL) was added N-cyclohexyl-N-methyl-cyclohexanamine (1.06 g, 5.44 mmol) and 2-(chloromethoxyethyl)trimethylsilane (680.44 mg, 4.08 mmol) at 20° C. The mixture was stirred at 20° C. for 12 hrs. The reaction was filtered and the filtrate was concentrated. The residue was purified by flash silica gel chromatography (ISCO; 20 g SepaFlash column) using a 0-6% EtOAc/petroleum ether gradient eluent to afford the title compound (950 mg, 98%) as a yellow oil. MS-ESI (m/z) calc'd for C₁₃H₁₉BrN₃O₂Si [M+H]⁺: 356.0/358.0. Found 355.9/357.9.

### Step 4: 5-(5-Bromo-1-((2-(trimethylsilyl)ethoxy)methyl)-1H-pyrazolo[3,4-c]pyridin-3-yl)oxazole

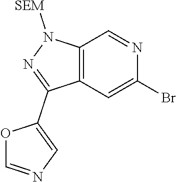 [SMILES: C[Si](C)(C)CCOCn1nc(-c2cnco2)c2cc(Br)ncc21]

To a solution of 5-bromo-1-((2-(trimethylsilyl)ethoxy)methyl)-1H-pyrazolo[3,4-c]pyridine-3-carbaldehyde (850 mg, 2.39 mmol) in MeOH (10 mL) was added K₂CO₃ (494.59 mg, 3.58 mmol) and TosMIC (465.78 mg, 2.39 mmol) at 20° C. The mixture was stirred at 65° C. for 1 hr. The reaction was filtered and the filtrate was concentrated. The residue was purified by flash silica gel chromatography (ISCO; 20 g SepaFlash column) using a 0-17% EtOAc/petroleum ether gradient eluent to afford the title compound (810 mg, 86%) as a yellow oil. MS-ESI (m/z) calc'd for C₁₅H₁₉BrN₄O₂Si [M+H]⁺: 395.1/397.1. Found 395.0/397.0.

### Step 5: 3-(Oxazol-5-yl)-1-((2-(trimethylsilyl)ethoxy)methyl)-1H-pyrazolo[3,4-c]pyridin-5-ol

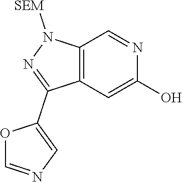 [SMILES: C[Si](C)(C)CCOCn1nc(-c2cnco2)c2cc(O)ncc21]

A mixture of 5-(5-bromo-1-((2-(trimethylsilyl)ethoxy)methyl)-1H-pyrazolo[3,4-c]pyridin-3-yl)oxazole (100 mg, 252.96 umol), KOH (85.15 mg, 1.52 mmol), Pd₂dba₃ (11.58 mg, 12.65 umol) and di-tert-butyl-[2-[2,4,6-tri(propan-2-yl)phenyl]phenyl]phosphane (10.74 mg, 25.30 umol) in dioxane (2 mL) and H₂O (2 mL) was degassed and purged with N₂ (3×) at 20° C. The mixture was stirred at 60° C. for 12 hrs under an N₂ atmosphere and then filtered.

The filtrate was adjusted to pH=5 with AcOH, diluted with H₂O, and extracted with EtOAc (3×). This procedure was conducted 2 additional times and the extracts were combined. The combined organic phases were dried with anhydrous Na₂SO₄ and concentrated to give a residue. The residue was purified by preparative-TLC (SiO₂, petroleum ether/[EtOAc/EtOH=3:1]=1:1, Rf=0.51) to afford the title compound (50 mg, 19%) as a yellow solid. MS-ESI (m/z) calc'd for C₁₅H₂₁N₄O₃Si [M+H]⁺: 333.1. Found 331.1.

### Step 6: 5-((3-(Oxazol-5-yl)-1-((2-(trimethylsilyl)ethoxy)methyl)-1H-pyrazolo[3,4-c]pyridin-5-yl)oxy)-5,6,7,8-tetrahydronaphthalene-2-carbonitrile

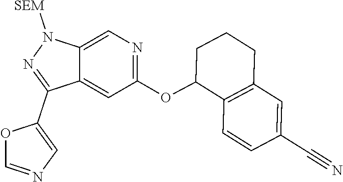 [SMILES: C[Si](C)(C)CCOCn1nc(-c2cnco2)c2cc(OC3CCCc4cc(C#N)ccc43)ncc21]

To a solution of 3-(oxazol-5-yl)-1-((2-(trimethylsilyl)ethoxy)methyl)-1H-pyrazolo[3,4-c]pyridin-5-ol (60 mg, 180.49 umol) and 5-hydroxy-5,6,7,8-tetrahydronaphthalene-2-carbonitrile (31.26 mg, 180.49 umol) in THF (3 mL) was added tributylphosphine (73.03 mg, 360.98 umol) and N-(piperidine-1-carbonylimino)piperidine-1-carboxamide (91.08 mg, 360.98 umol) at 0° C. The mixture was stirred at 45° C. for 1 hr. The reaction was filtered and the filtrate was concentrated. The residue was purified by preparative-TLC (SiO₂, petroleum ether/[EtOAc/EtOH=3:1]=1:1, Rf=0.30) to afford the title compound (30 mg, 34%) as a yellow solid. MS-ESI (m/z) calc'd for C₂₆H₃₀N₅O₃Si [M+H]⁺: 488.2. Found 488.2.

### Step 7: 5-((3-(Oxazol-5-yl)-1H-pyrazolo[3,4-c]pyridin-5-yl)oxy)-5,6,7,8-tetrahydronaphthalene-2-carbonitrile

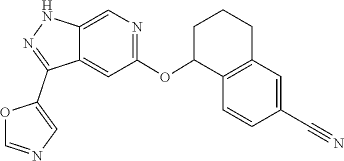 [SMILES: N#Cc1ccc2c(c1)CCCC2Oc1cc2c(-c3cnco3)n[nH]c2cn1]

To a solution of 5-((3-(oxazol-5-yl)-1-((2-(trimethylsilyl)ethoxy)methyl)-1H-pyrazolo[3,4-c]pyridin-5-yl)oxy)-5,6,7,8-tetrahydronaphthalene-2-carbonitrile (10 mg, 20.51 umol) in THF (0.5 mL) was added ethane-1,2-diamine (6.16 mg, 102.54 umol) and tetrabutylammonium fluoride (1 M, 102.54 uL) at 20° C. The mixture was stirred at 60° C. for 12 hrs. The mixture was concentrated to give a residue. This procedure was conducted a second time and the residues were combined and purified by preparative-HPLC using Method AI to afford the title compound (9 mg, 61%) as a white solid. MS-ESI (m/z) calc'd for C₂₀H₁₆N₅O₂ [M+H]⁺: 358.1. Found 358.0.

### Step 8: 5-((3-(Oxazol-5-yl)-1H-pyrazolo[3,4-c]pyridin-5-yl)oxy)-5,6,7,8-tetrahydronaphthalene-2-carbonitrile, enantiomer 1 and 2

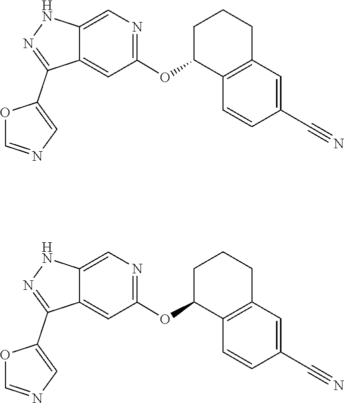 [SMILES: N#Cc1ccc2c(c1)CCC[C@H]2Oc1cc2c(-c3cnco3)n[nH]c2cn1 ; N#Cc1ccc2c(c1)CCC[C@@H]2Oc1cc2c(-c3cnco3)n[nH]c2cn1]

5-((3-(Oxazol-5-yl)-1H-pyrazolo[3,4-c]pyridin-5-yl)oxy)-5,6,7,8-tetrahydronaphthalene-2-carbonitrile was subjected to chiral separation using Method AJ to afford 5-((3-(oxazol-5-yl)-1H-pyrazolo[3,4-c]pyridin-5-yl)oxy)-5,6,7,8-tetrahydronaphthalene-2-carbonitrile, enantiomer 1 (3.97 mg, 58%). ¹H NMR (400 MHz, DMSO-d₆) δ 8.83 (d, J=0.98 Hz, 1H), 8.50 (s, 1H), 7.83 (s, 1H), 7.69 (s, 1H), 7.58-7.63 (m, 1H), 7.50-7.56 (m, 1H), 7.37 (d, J=1.10 Hz, 1H), 6.27 (t, J=5.26 Hz, 1H), 2.76-2.96 (m, 2H), 2.08-2.16 (m, 1H), 1.91-2.05 (m, 2H), 1.79-1.87 (m, 1H). MS-ESI (m/z) calc'd for C₂₀H₁₆N₅O₂ [M+H]⁺: 358.1. Found 358.0. A later eluting fraction was also isolated to afford 5-((3-(oxazol-5-yl)-1H-pyrazolo[3,4-c]pyridin-5-yl)oxy)-5,6,7,8-tetrahydronaphthalene-2-carbonitrile, enantiomer 2 (2.68 mg, 39%). ¹H NMR (400 MHz, DMSO-d₆) δ 8.84 (d, J=0.98 Hz, 1H), 8.50 (s, 1H), 7.83 (s, 1H), 7.69 (s, 1H), 7.58-7.64 (m, 1H), 7.49-7.57 (m, 1H), 7.37 (d, J=0.98 Hz, 1H), 6.27 (t, J=5.20 Hz, 1H), 2.75-2.98 (m, 2H), 2.08-2.17 (m, 1H), 1.93-2.06 (m, 2H), 1.80-1.86 (m, 1H). MS-ESI (m/z) calc'd for C₂₀H₁₆N₅O₂ [M+H]⁺: 358.1. Found 358.1.

### Example 33: 5-((3-(Oxazol-5-yl)-1H-pyrazolo[3,4-c]pyridin-5-yl)amino)-5,6,7,8-tetrahydronaphthalene-2-carbonitrile, enantiomer 1 and 2

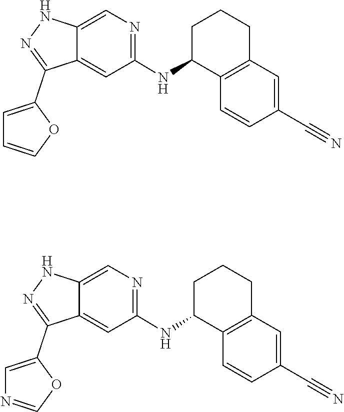 [SMILES: N#Cc1ccc2c(c1)CCC[C@@H]2Nc1cc2c(-c3ccco3)n[nH]c2cn1 ; N#Cc1ccc2c(c1)CCC[C@H]2Nc1cc2c(-c3cnco3)n[nH]c2cn1]

### Step 1: 5-Amino-5,6,7,8-tetrahydronaphthalene-2-carbonitrile

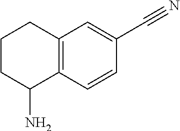 [SMILES: N#Cc1ccc2c(c1)CCCC2N]

To a solution of 1-oxotetralin-6-carbonitrile (1.2 g, 7.00 mmol) in MeOH (10 mL) in a microwave reaction tube was added NH₄OAc (8.1 g, 105.14 mmol) and the mixture was stirred at 20° C. for 10 min. Then NaBH₃CN (1.76 g, 28.04 mmol) was added to the mixture.

The tube was sealed and heated at 90° C. for 30 min under microwave irradiation. The reaction was filtered and the filtrate was concentrated. The residue was diluted with H₂O, acidified with TN HCl to pH=3 and extracted with EtOAc (5×). The organic phase was discarded. The aqueous phase was basified with solid NaHCO₃ to pH=8 and extracted with CH₂Cl₂ (5×). The combined organic phases were dried with anhydrous Na₂SO₄ and evaporated to dryness to afford the title compound (330 mg, 27%) as a colorless oil. MS-ESI (m/z) calc'd for C₁₁H₁₃N₂ [M+H]⁺: 173.1. Found 173.1.

### Step 2: 5-((3-(Oxazol-5-yl)-1-((2-(trimethylsilyl)ethoxy)methyl)-H-pyrazolo[3,4-c]pyridin-5-yl)amino)-5,6,7,8-tetrahydronaphthalene-2-carbonitrile

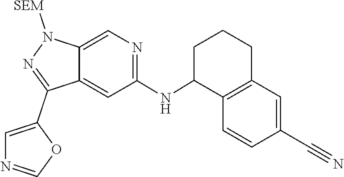 [SMILES: C[Si](C)(C)CCOCn1nc(-c2cnco2)c2cc(NC3CCCc4cc(C#N)ccc43)ncc21]

To a solution of 2-[(5-bromo-3-oxazol-5-yl-pyrazolo[3,4-c]pyridin-1-yl)methoxy]ethyl-trimethyl-silane (100 mg, 252.96 umol) in THF (4.8 mL) was added 5-amino-5,6,7,8-tetrahydronaphthalene-2-carbonitrile (114 mg, 661.92 umol) and tBuBrettPhos Pd G3 (26.29 mg, 33.10 umol) at 20° C. Then a solution of NaOtBu (2 M, 330.96 uL) in THF (0.2 mL) was added. The mixture was stirred at 60° C. for 12 hrs under an N₂ atmosphere. The mixture was evaporated to give a residue and the residue was purified by flash silica gel chromatography (ISCO; 20 g SepaFlash column) using a 0-16% EtOAc/petroleum ether gradient eluent to afford the title compound (76 mg, 24%) as a green oil. MS-ESI (m/z) calc'd for C₂₆H₃₁N₆O₂Si [M+H]⁺: 487.2. Found 487.2.

### Step 3: 5-((3-(Oxazol-5-yl)-H-pyrazolo[3,4-c]pyridin-5-yl)amino)-5,6,7,8-tetrahydronaphthalene-2-carbonitrile

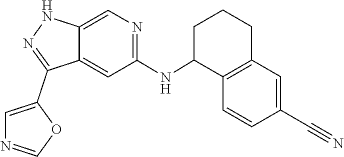 [SMILES: N#Cc1ccc2c(c1)CCCC2Nc1cc2c(-c3cnco3)n[nH]c2cn1]

To a solution of 5-((3-(oxazol-5-yl)-1-((2-(trimethylsilyl)ethoxy)methyl)-1H-pyrazolo[3,4-c]pyridin-5-yl)amino)-5,6,7,8-tetrahydronaphthalene-2-carbonitrile (95 mg, 195.22 umol) in THF (4 mL) was added ethane-1,2-diamine (117.32 mg, 1.95 mmol) and tetrabutylammonium fluoride (1 M, 1.95 mL) at 20° C. The mixture was stirred at 60° C. for 12 hrs. The mixture was evaporated to give a residue and the residue was purified by preparative-HPLC using Method AK to afford the title compound (10 mg, 11%) as a yellow solid. MS-ESI (m/z) calc'd for C₂₀H₁₇N₆O [M+H]⁺: 357.1. Found 357.1.

### Step 4: 5-((3-(Oxazol-5-yl)-1H-pyrazolo[3,4-c]pyridin-5-yl)amino)-5,6,7,8-tetrahydronaphthalene-2-carbonitrile, enantiomer 1 and 2

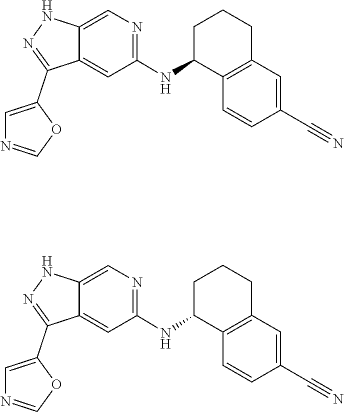 [SMILES: N#Cc1ccc2c(c1)CCC[C@@H]2Nc1cc2c(-c3cnco3)n[nH]c2cn1 ; N#Cc1ccc2c(c1)CCC[C@H]2Nc1cc2c(-c3cnco3)n[nH]c2cn1]

5-((3-(Oxazol-5-yl)-1H-pyrazolo[3,4-c]pyridin-5-yl)amino)-5,6,7,8-tetrahydronaphthalene-2-carbonitrile was subjected to chiral separation using Method AL to afford 5-((3-(oxazol-5-yl)-1H-pyrazolo[3,4-c]pyridin-5-yl)amino)-5,6,7,8-tetrahydronaphthalene-2-carbonitrile, enantiomer 1 (4.03 mg, 45%). ¹H NMR (400 MHz, DMSO-d₆) δ 12.49-13.72 (m, 1H), 8.69 (d, J=1.10 Hz, 1H), 8.53 (s, 1H), 7.61-7.64 (m, 2H), 7.52-7.56 (m, 1H), 7.45-7.49 (m, 1H), 6.98 (d, J=1.22 Hz, 1H), 6.58 (d, J=9.05 Hz, 1H), 5.21-5.30 (m, 1H), 2.75-2.91 (m, 2H), 1.91-2.11 (m, 2H), 1.71-1.89 (m, 2H). MS-ESI (m/z) calc'd for C₂₀H₁₇N₆O [M+H]⁺: 357.1. Found 357.1. A later eluting fraction was also isolated to afford 5-((3-(oxazol-5-yl)-1H-pyrazolo[3,4-c]pyridin-5-yl)amino)-5,6,7,8-tetrahydronaphthalene-2-carbonitrile, enantiomer 2 (4.05 mg, 45%). ¹H NMR (400 MHz, DMSO-d₆) δ 13.51 (br s, 1H), 8.69 (d, J=0.98 Hz, 1H), 8.53 (s, 1H), 7.60-7.64 (m, 2H), 7.52-7.56 (m, 1H), 7.45-7.50 (m, 1H), 6.98 (d, J=1.22 Hz, 1H), 6.57 (d, J=9.05 Hz, 1H), 5.21-5.30 (m, 1H), 2.78-2.90 (m, 2H), 1.89-2.08 (m, 2H), 1.74-1.88 (m, 2H). MS-ESI (m/z) calc'd for C₂₀H₁₇N₆O [M+H]⁺: 357.1. Found 357.1.

### Example 34: 6,6-Difluoro-7-((3-(oxazol-5-yl)-1H-indazol-5-yl)amino)-6,7-dihydro-5H-cyclopenta[b]pyridine-3-carbonitrile

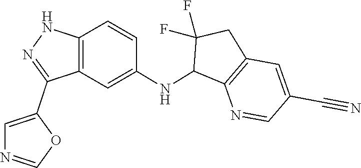 [SMILES: N#Cc1cnc2c(c1)CC(F)(F)C2Nc1ccc2[nH]nc(-c3cnco3)c2c1]

### Step 1: 3-Bromo-6,7-dihydro-5H-cyclopenta[b]pyridine 1-oxide

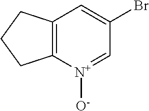 [SMILES: [O-][n+]1cc(Br)cc2c1CCC2]

To a solution of 3-bromo-6,7-dihydro-5H-cyclopenta[b]pyridine (12 g, 60.59 mmol) in 1,2-dichloroethane (360 mL) was added MCPBA (24.60 g, 121.18 mmol) at 20° C. and the mixture was stirred at 70° C. for 12 hrs. The reaction mixture was diluted with DCM and basified with saturated aqueous Na₂CO₃ to pH=8. The organic layer was separated and the aqueous phase was extracted with DCM (3×). The combined organic layers were dried over Na₂SO₄ and evaporated to dryness to afford the title compound (10.4 g, 80%) as a black solid. MS-ESI (m/z) calc'd for C₈H₉BrNO [M+H]⁺: 214.0/216.0. Found 213.9.1/215.9.

### Step 2: 3-Bromo-6,7-dihydro-5H-cyclopenta[b]pyridin-7-ol

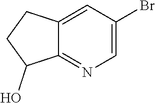 [SMILES: OC1CCc2cc(Br)cnc21]

A mixture of 3-bromo-6,7-dihydro-5H-cyclopenta[b]pyridine 1-oxide (8.4 g, 39.24 mmol) and TFAA (57.69 g, 274.69 mmol) was stirred at 50° C. for 1 hr. The reaction mixture was evaporated to give a residue and the residue was purified by flash silica gel chromatography (ISCO; 40 g SepaFlash column) using a 0-40% EtOAc/petroleum ether gradient eluent to afford the title compound (6.5 g, 77%) as a green solid. ¹H NMR (400 MHz, CHLOROFORM-d) δ 8.42 (s, 1H), 7.62-7.66 (m, 1H), 5.10 (dd, J=7, 6 Hz, 1H), 4.21 (br s, 1H), 2.91-3.01 (m, 1H), 2.71-2.81 (m, 1H), 2.44-2.54 (m, 1H), 1.95-2.06 (m, 1H). MS-ESI (m/z) calc'd for C₈H₉BrNO [M+H]⁺: 214.0/216.0. Found 213.9.1/215.9.

### Step 3: 3-Bromo-5H-cyclopenta[b]pyridin-7(6H)-one

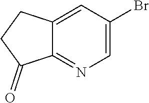 [SMILES: O=C1CCc2cc(Br)cnc21]

To a solution of 3-bromo-6,7-dihydro-5H-cyclopenta[b]pyridin-7-ol (2 g, 9.34 mmol) in DCM (100 mL) was added Dess-Martin periodinane (4.76 g, 11.21 mmol) and the mixture was stirred at 20° C. for 2 hrs. The reaction mixture was then diluted with saturated aqueous Na₂CO₃ to pH=8, and extracted with DCM (3×). The combined organic layers were dried over Na₂SO₄ and evaporated to afford a residue. The residue was purified by flash silica gel chromatography (ISCO; 20 g SepaFlash column) using a 0-50% EtOAc/petroleum ether gradient eluent to afford the title compound (1.4 g, 71%) as a gray solid. ¹H NMR (400 MHz, CDCl₃) δ 8.80 (s, 1H), 8.05 (d, J=1 Hz, 1H), 3.13-3.20 (m, 2H), 2.74-2.80 (m, 2H). MS-ESI (m/z) calc'd for C₈H₇BrNO [M+H]⁺: 212.0/214.0. Found 211.9/213.9.

### Step 4: 3-Bromo-6,6-difluoro-5,6-dihydro-7H-cyclopenta[b]pyridin-7-one

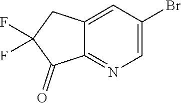 [SMILES: O=C1c2ncc(Br)cc2CC1(F)F]

To a mixture of NaH (784.67 mg, 19.62 mmol) in THF (30 mL) was added a solution of 3-bromo-5H-cyclopenta[b]pyridin-7(6H)-one (1.3 g, 6.13 mmol) in THF (20 mL) at 0° C. The mixture was stirred at 0° C. for 10 minutes, then 1-chloromethyl-4-fluoro-1,4-diazoniabicyclo[2.2.2]octane bis(tetrafluoroborate) (4.56 g, 12.87 mmol) was added. The mixture was allowed to warm to 20° C. and stirred for an additional 1 hr. The reaction mixture was quenched with H₂O and extracted with EtOAc (3×). The combined organic layers were dried over Na₂SO₄ and evaporated to give a residue. The residue was purified by flash silica gel chromatography (ISCO; 20 g SepaFlash column) using a 0-25% EtOAc/petroleum ether gradient eluent to afford the title compound (490 mg, 32%) as a yellow solid. ¹H NMR (400 MHz, CDCl₃) δ 8.87 (s, 1H), 8.01 (s, 1H), 3.54 (t, J=12 Hz, 2H). MS-ESI (m/z) calc'd for C₈H₅BrF₂NO [M+H]⁺: 247.9/249.9. Found 247.9/249.9.

### Step 5: N-(3-Bromo-6,6-difluoro-6,7-dihydro-5H-cyclopenta[b]pyridin-7-yl)-3-(oxazol-5-yl)-JH-indazol-5-amine

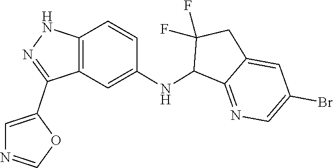 [SMILES: FC1(F)Cc2cc(Br)cnc2C1Nc1ccc2[nH]nc(-c3cnco3)c2c1]

To a solution of 3-oxazol-5-yl-1H-indazol-5-amine (80 mg, 399.61 umol) and 3-bromo-6,6-difluoro-5,6-dihydro-7H-cyclopenta[b]pyridin-7-one (99.11 mg, 399.61 umol) in toluene (4 mL) was added Ti(Oi-Pr)₄ (567.87 mg, 2.00 mmol) and the mixture was stirred at 100° C. for 4 hrs. After cooling to 20° C., the mixture was evaporated to give a residue. The residue was dissolved in MeOH (4 mL) and NaBH₄ (120.94 mg, 3.20 mmol) was added at 0° C. The mixture was then stirred at 20° C. for an additional 4 hrs. The reaction mixture was filtered and the filtrate was evaporated to give a residue. The residue was purified by preparative-TLC (SiO₂, 100% petroleum ether, Rf=0.33) to afford the title compound (80 mg, 46%) as an orange solid. MS-ESI (m/z) calc'd for C₁₈H₁₃BrF₂N₅O [M+H]⁺: 432.0/434.0. Found 432.0/434.0.

### Step 6: 6,6-Difluoro-7-((3-(oxazol-5-yl)-1H-indazol-5-yl)amino)-6,7-dihydro-5H-cyclopenta[b]pyridine-3-carbonitrile

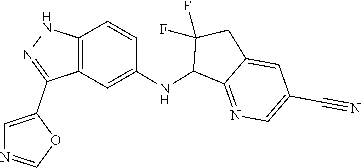 [SMILES: N#Cc1cnc2c(c1)CC(F)(F)C2Nc1ccc2[nH]nc(-c3cnco3)c2c1]

A mixture of N-(3-bromo-6,6-difluoro-6,7-dihydro-5H-cyclopenta[b]pyridin-7-yl)-3-(oxazol-5-yl)-1H-indazol-5-amine (30 mg, 69.41 umol), tetrapotassium hexacyanoferrate trihydrate (14.66 mg, 34.70 umol), XPhos (1.65 mg, 3.47 umol), 0.05 M aqueous KOAc (180.46 uL, 9.02 umol) (aqueous solution), XantPhos Pd G3 (3.29 mg, 3.47 umol) in 1,4-dioxane (0.18 mL) was degassed and purged with N₂ (3×) at 20° C., and then the mixture was stirred at 100° C. for 1 hr under N₂ atmosphere. The reaction mixture was evaporated to give a residue that was purified by preparative-HPLC using Method BA to afford the title compound (2.73 mg, 7%) as a green solid. ¹H NMR (400 MHz, DMSO-d₆) δ 13.22 (br s, 1H), 9.02 (s, 1H), 8.52-8.55 (m, 1H), 8.37 (s, 1H), 7.70 (s, 1H), 7.48 (d, J=9 Hz, 1H), 7.31 (s, 1H), 7.21 (dd, J=9, 2 Hz, 1H), 6.34 (br s, 1H), 5.78-5.89 (m, 1H), 3.61-3.78 (m, 2H). MS-ESI (m/z) calc'd for C₁₉H₁₃F₂N₆O [M+H]⁺: 379.0. Found 379.0.

### Example 35: 4-((3-(Oxazol-5-yl)-1H-indazol-5-yl)amino)-4,5,6,7-tetrahydrobenzo[d]oxazole-2-carbonitrile, enantiomer 1 and 2

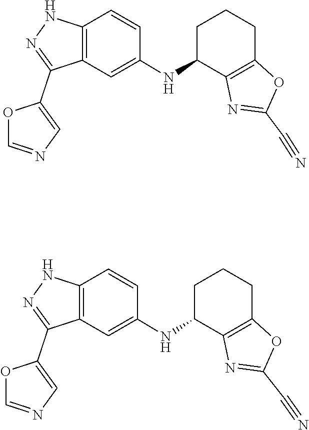 [SMILES: N#Cc1nc2c(o1)CCC[C@@H]2Nc1ccc2[nH]nc(-c3cnco3)c2c1 ; N#Cc1nc2c(o1)CCC[C@H]2Nc1ccc2[nH]nc(-c3cnco3)c2c1]

### Step 1: 2-Diazocyclohexane-1,3-dione

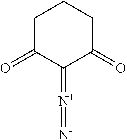

To a solution of cyclohexane-1,3-dione (5 g, 44.59 mmol) and N-(4-azidosulfonylphenyl)acetamide (10.71 g, 44.59 mmol) in MeCN (240 mL) was added Et₃N (4.96 g, 49.05 mmol) at 15° C. The mixture was stirred at 15° C. for 1 hr. The mixture was filtered and the solid was washed with DCM (1×), the filtrate was evaporated to give a residue that was purified by flash silica gel chromatography (ISCO; 25 g SepaFlash column) using a 0-16% EtOAc/petroleum ether gradient eluent to afford the title compound (6 g, 97%) as a pale yellow solid. ¹H NMR (400 MHz, CDCl₃) δ 2.50-2.58 (m, 4H), 1.99-2.07 (m, 2H). MS-ESI (m/z) calc'd for C₆H₇N₂O₂ [M+H]⁺: 139.0. Found 139.1.

### Step 2: Ethyl 4-oxo-4,5,6,7-tetrahydrobenzo[d]oxazole-2-carboxylate

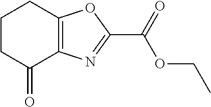

To a solution of 2-diazocyclohexane-1,3-dione (6 g, 43.44 mmol) in ethyl cyanoformate (15.15 g, 152.89 mmol) was added Rh₂(OAc)₄ (383.99 mg, 868.79 umol) at 15° C. The mixture was stirred at 60° C. for 12 hrs. The reaction mixture was concentrated and the residue was purified by flash silica gel chromatography (ISCO; 25 g SepaFlash column) using a 0-16% EtOAc/petroleum ether gradient eluent to afford the title compound (2.4 g, 26%) as a pale yellow solid. ¹H NMR (400 MHz, CDCl₃) δ 4.32 (qd, J=7.07, 2.44 Hz, 2H), 2.94 (td, J=6.16, 2.31 Hz, 2H), 2.47-2.52 (m, 2H), 2.11-2.19 (m, 2H), 1.28 (td, J=7.07, 2.38 Hz, 3H). MS-ESI (m/z) calc'd for C₁₀H₁₂NO₄ [M+H]⁺: 210.1. Found 210.1.

### Step 3: 4-Oxo-4,5,6,7-tetrahydrobenzo[d]oxazole-2-carboxamide

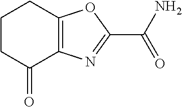 [SMILES: NC(=O)c1nc2c(o1)CCCC2=O]

A solution of ethyl 4-oxo-4,5,6,7-tetrahydrobenzo[d]oxazole-2-carboxylate (2.4 g, 11.47 mmol) in NH₄OH (13.65 g, 15 mL, 25%) was stirred at 40° C. for 1 hr. The mixture was filtered and the solid was dried under reduced pressure to afford the title compound (800 mg, 38%) as a white solid. MS-ESI (m/z) calc'd for C₈H₉N₂O₃ [M+H]⁺: 181.1. Found 181.0.

### Step 4:4-Oxo-4,5,6,7-tetrahydrobenzo[d]oxazole-2-carbonitrile

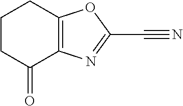 [SMILES: N#Cc1nc2c(o1)CCCC2=O]

To a solution of 4-oxo-4,5,6,7-tetrahydrobenzo[d]oxazole-2-carboxamide (800 mg, 4.44 mmol) in THF (12 mL) was added TFAA (4.66 g, 22.20 mmol) and Et₃N (898.66 mg, 8.88 mmol) at 0° C. The mixture was stirred at 15° C. for 12 hrs. The reaction mixture was then evaporated to give a residue that was purified by flash silica gel chromatography (ISCO; 12 g SepaFlash column) using a 0-25% EtOAc/petroleum ether gradient eluent to afford the title compound (500 mg, 69%) as a yellow oil. ¹H NMR (400 MHz, CDCl₃) δ 3.02 (t, J=6.30 Hz, 2H), 2.56-2.65 (m, 2H), 2.20-2.30 (m, 2H). MS-ESI (m/z) calc'd for C₈H₇N₂O₂ [M+H]⁺: 163.0. Found 163.0.

### Step 5: 4-((3-(Oxazol-5-yl)-1H-indazol-5-yl)amino)-4,5,6,7-tetrahydrobenzo[d]oxazole-2-carbonitrile

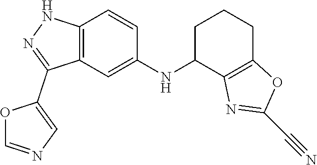 [SMILES: N#Cc1nc2c(o1)CCCC2Nc1ccc2[nH]nc(-c3cnco3)c2c1]

To a solution of 3-oxazol-5-yl-1H-indazol-5-amine (100 mg, 499.51 umol) 4-oxo-4,5,6,7-tetrahydrobenzo[d]oxazole-2-carbonitrile (80.99 mg, 499.51 umol) in MeOH (1 mL) was added AcOH (59.99 mg, 999.02 umol) to adjust pH=5 and the mixture was stirred at 15° C. for 1 hr. NaBH₃CN (94.17 mg, 1.50 mmol) was then added and the mixture was stirred at 15° C. for 13 hrs. The reaction mixture was concentrated and the residue was purified by preparative-HPLC using Method AM to afford the title compound (100 mg, 43%) as a pale yellow solid. MS-ESI (m/z) calc'd for C₁₈H₁₅N₆O₂ [M+H]⁺: 347.1. Found 347.1.

### Step 6: 4-((3-(Oxazol-5-yl)-1H-indazol-5-yl)amino)-4,5,6,7-tetrahydrobenzo[d]oxazole-2-carbonitrile, enantiomer 1 and 2

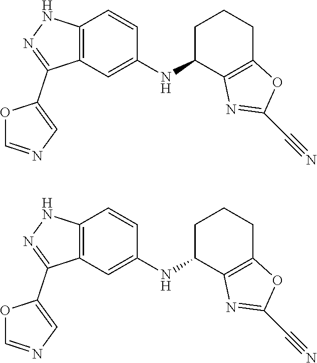 [SMILES: N#Cc1nc2c(o1)CCC[C@@H]2Nc1ccc2[nH]nc(-c3cnco3)c2c1 ; N#Cc1nc2c(o1)CCC[C@H]2Nc1ccc2[nH]nc(-c3cnco3)c2c1]

4-((3-(Oxazol-5-yl)-1H-indazol-5-yl)amino)-4,5,6,7-tetrahydrobenzo[d]oxazole-2-carbonitrile was subjected to chiral separation using Method AN to afford 4-((3-(oxazol-5-yl)-1H-indazol-5-yl)amino)-4,5,6,7-tetrahydrobenzo[d]oxazole-2-carbonitrile, enantiomer 1 (3.59 mg, 37%) as a yellow solid. ¹H NMR (400 MHz, MeOD) δ 8.34 (s, 1H), 7.61 (s, 1H), 7.40 (d, J=9.03 Hz, 1H), 7.24 (d, J=1.63 Hz, 1H), 7.05 (dd, J=2.07, 8.97 Hz, 1H), 4.68-4.73 (m, 1H), 2.63-2.93 (m, 2H), 1.90-2.17 (m, 4H). MS-ESI (m/z) calc'd for C₁₈H₁₅N₆O₂ [M+H]⁺: 347.1. Found 347.1. A later eluting fraction was also isolated to afford 4-((3-(oxazol-5-yl)-1H-indazol-5-yl)amino)-4,5,6,7-tetrahydrobenzo[d]oxazole-2-carbonitrile, enantiomer 2 (1.75 mg, 18%) as a yellow solid. ¹H NMR (400 MHz, MeOD) δ 8.34 (s, 1H), 7.61 (s, 1H), 7.40 (d, J=9.03 Hz, 1H), 7.24 (d, J=1.88 Hz, 1H), 7.05 (dd, J=2.07, 8.97 Hz, 1H), 4.71 (br s, 1H), 2.65-2.91 (m, 2H), 1.92-2.17 (m, 4H). MS-ESI (m/z) calc'd for C₁₈H₁₅N₆O₂ [M+H]⁺: 347.1. Found 347.1.

### Example 36: 4-((3-(Oxazol-5-yl)-1H-indazol-5-yl)amino)-5,6-dihydro-4H-cyclopenta[d]oxazole-2-carbonitrile, enantiomer 1 and 2

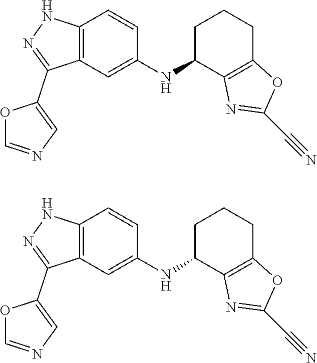 [SMILES: N#Cc1nc2c(o1)CCC[C@@H]2Nc1ccc2[nH]nc(-c3cnco3)c2c1 ; N#Cc1nc2c(o1)CCC[C@H]2Nc1ccc2[nH]nc(-c3cnco3)c2c1]

4-((3-(Oxazol-5-yl)-1H-indazol-5-yl)amino)-5,6-dihydro-4H-cyclopenta[d]oxazole-2-carbonitrile was subjected to chiral separation using Method AQ to afford 4-((3-(oxazol-5-yl)-1H-indazol-5-yl)amino)-5,6-dihydro-4H-cyclopenta[d]oxazole-2-carbonitrile, enantiomer 1 (3.4 mg, 37%) as a yellow solid. ¹H NMR (400 MHz, DMSO-d₆) δ 13.18 (br s, 1H), 8.54 (s, 1H), 7.73 (s, 1H), 7.45 (d, J=9 Hz, 1H), 7.18 (s, 1H), 7.01 (dd, J=9, 2 Hz, 1H), 6.04 (d, J=8 Hz, 1H), 4.98 (br s, 1H), 3.12-3.23 (m, 1H), 2.99-3.10 (m, 1H), 2.87-2.98 (m, 1H), 2.47 (ddd, J=13, 9, 4 Hz, 1H). MS-ESI (m/z) calc'd for C₁₇H₁₃N₆O₂ [M+H]⁺: 333.1. Found 333.0. A later eluting fraction was also isolated to afford 4-((3-(oxazol-5-yl)-1H-indazol-5-yl)amino)-5,6-dihydro-4H-cyclopenta[d]oxazole-2-carbonitrile, enantiomer 2 (3.09 mg, 33%) as a yellow solid. ¹H NMR (400 MHz, DMSO-d₆) δ 13.13 (br s, 1H), 8.48 (s, 1H), 7.67 (s, 1H), 7.40 (d, J=9 Hz, 1H), 7.12 (s, 1H), 6.96 (dd, J=9, 2 Hz, 1H), 5.98 (d, J=8 Hz, 1H), 4.93 (br s, 1H), 3.07-3.18 (m, 1H), 2.94-3.05 (m, 1H), 2.82-2.91 (m, 1H), 2.41 (ddd, J=13, 9, 5 Hz, 1H). MS-ESI (m/z) calc'd for C₁₇H₁₃N₆O₂ [M+H]⁺: 333.1. Found 333.0.

### Example 37: 3-((3-(Oxazol-5-yl)-1H-indazol-5-yl)oxy)-2,3-dihydro-1H-indene-4-carbonitrile, enantiomer 1 and 2

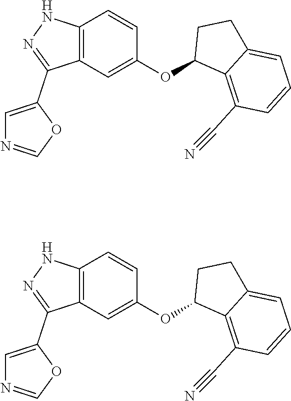 [SMILES: N#Cc1cccc2c1[C@@H](Oc1ccc3[nH]nc(-c4cnco4)c3c1)CC2 ; N#Cc1cccc2c1[C@H](Oc1ccc3[nH]nc(-c4cnco4)c3c1)CC2]

### Step 1: 3-Oxo-2,3-dihydro-1H-indene-4-carbonitrile

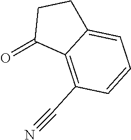 [SMILES: N#Cc1cccc2c1C(=O)CC2]

7-Bromoindan-1-one (500 mg, 2.37 mmol) and CuCN (275.83 mg, 3.08 mmol) were taken up in a microwave tube in NMP (5 mL) at 20° C. The sealed tube was heated at 190° C. for 1 hr under microwave irradiation. The reaction mixture was diluted with H₂O and extracted with EtOAc (4×). The combined organic layers were concentrated and the residue was combined with another 0.02 g batch to afford the title compound (580 mg) as a brown solid. ¹H NMR (400 MHz, CDCl₃) δ 7.65-7.79 (m, 3H), 3.16-3.23 (m, 2H), 2.77-2.81 (m, 2H). MS-ESI (m/z) calc'd for C₁₀H₈NO [M+H]⁺: 158.1. Found 158.0.

### Step 2: 3-Hydroxy-2,3-dihydro-1H-indene-4-carbonitrile

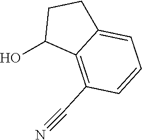 [SMILES: N#Cc1cccc2c1C(O)CC2]

To a solution of 3-oxo-2,3-dihydro-1H-indene-4-carbonitrile (560 mg, 3.56 mmol) in MeOH (3 mL) was added NaBH₄ (269.58 mg, 7.13 mmol) at 20° C. The mixture was stirred at 50° C. for 40 minutes. The reaction mixture was quenched by addition of saturated aqueous NH₄Cl at 20° C. and extracted with EtOAc (3×). The combined organic layers were concentrated to afford the title compound (290 mg, 51%) as a light brown gum. ¹H NMR (400 MHz, DMSO-d₆) δ 7.62 (dd, J=7.58, 0.73 Hz, 1H) 7.57 (dd, J=7.58, 0.61 Hz, 1H) 7.38-7.44 (m, 1H) 5.53 (d, J=6.97 Hz, 1H) 5.20 (td, J=6.91, 4.77 Hz, 1H) 2.98-3.08 (m, 1H) 2.71-2.83 (m, 1H) 2.30-2.41 (m, 1H) 1.88 (m, 1H).

### Step 3: 3-((3-(Oxazol-5-yl)-1-(tetrahydro-2H-pyran-2-yl)-1H-indazol-5-yl)oxy)-2,3-dihydro-1H-indene-4-carbonitrile

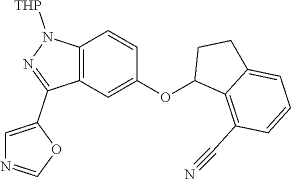

To a solution of 3-hydroxy-2,3-dihydro-1H-indene-4-carbonitrile (100 mg, 628.20 umol) and 3-oxazol-5-yl-1-tetrahydropyran-2-yl-indazol-5-ol (107.53 mg, 376.92 umol) in THF (4 mL) was added 1,1′-(azodicarbonyl)dipiperidine (317.01 mg, 1.26 mmol) and tributylphosphine (254.19 mg, 1.26 mmol) at 0° C. The mixture was stirred at 45° C. for 2 hrs. The reaction mixture was diluted with H₂O and extracted with EtOAc (4×). The combined organic layers were concentrated and the residue was purified by preparative-TLC (SiO₂, petroleum ether/EtOAc=1:4, Rf=0.40) to afford the title compound (67 mg, 25%) as a light yellow solid. MS-ESI (m/z) calc'd for C₂₅H₂₃N₄O₃ [M+H]⁺: 427.2. Found 427.2.

### Step 4: 3-((3-(Oxazol-5-yl)-1H-indazol-5-yl)oxy)-2,3-dihydro-1H-indene-4-carbonitrile

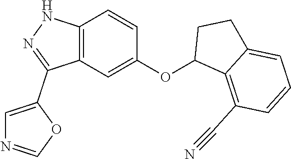 [SMILES: N#Cc1cccc2c1C(Oc1ccc3[nH]nc(-c4cnco4)c3c1)CC2]

To a solution of 3-((3-(oxazol-5-yl)-1-(tetrahydro-2H-pyran-2-yl)-1H-indazol-5-yl)oxy)-2,3-dihydro-1H-indene-4-carbonitrile (57 mg, 133.66 umol) in MeOH (5 mL) and H₂O (1 mL) was added p-toluenesulfonic acid (115.08 mg, 668.28 umol) at 20° C. The mixture was stirred at 70° C. for 3 hrs. The reaction mixture was then diluted with H₂O and extracted with EtOAc (4×). The combined organic layers were concentrated and the residue was combined with material from another 0.01 g batch. The residue was purified by preparative-TLC (SiO₂, 100% EtOAc, Rf=0.40) to afford the title compound (26 mg, 49%) as an off-white solid. MS-ESI (m/z) calc'd for C₂₀H₁₅N₄O₂ [M+H]⁺: 343.1. Found 343.1.

### Step 5: 3-((3-(Oxazol-5-yl)-1H-indazol-5-yl)oxy)-2,3-dihydro-1H-indene-4-carbonitrile, enantiomer 1 and 2

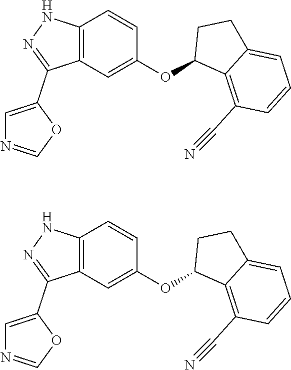 [SMILES: N#Cc1cccc2c1[C@@H](Oc1ccc3[nH]nc(-c4cnco4)c3c1)CC2 ; N#Cc1cccc2c1[C@H](Oc1ccc3[nH]nc(-c4cnco4)c3c1)CC2]

3-((3-(Oxazol-5-yl)-1H-indazol-5-yl)oxy)-2,3-dihydro-1H-indene-4-carbonitrile was subjected to chiral separation using Method AX to afford 3-((3-(oxazol-5-yl)-1H-indazol-5-yl)oxy)-2,3-dihydro-1H-indene-4-carbonitrile, enantiomer 1 (2.01 mg, 21%) as a white solid: ¹H NMR (400 MHz, DMSO-d₆) δ 13.42 (br s, 1H), 8.52 (s, 1H), 7.84 (s, 1H), 7.77 (br d, J=7.58 Hz, 1H), 7.72 (br d, J=7.70 Hz, 1H), 7.53-7.61 (m, 3H), 7.15-7.21 (m, 1H), 6.14 (dd, J=6.54, 3.24 Hz, 1H), 3.11-3.21 (m, 1H), 2.96-3.06 (m, 1H), 2.64-2.76 (m, 1H), 2.19 (td, J=8.93, 4.16 Hz, 1H). MS-ESI (m/z) calc'd for C₂₀H₁₅N₄O₂ [M+H]⁺: 343.1. Found 343.1. A later eluting fraction was also isolated to afford 3-((3-(oxazol-5-yl)-1H-indazol-5-yl)oxy)-2,3-dihydro-1H-indene-4-carbonitrile, enantiomer 2 (2.33 mg, 25%) as a white solid: ¹H NMR (400 MHz, DMSO-d₆) δ 13.42 (br s, 1H), 8.52 (s, 1H), 7.84 (s, 1H), 7.77 (d, J=7.58 Hz, 1H), 7.72 (d, J=7.46 Hz, 1H), 7.53-7.62 (m, 3H), 7.18 (dd, J=9.17, 2.08 Hz, 1H), 6.14 (dd, J=6.72, 3.42 Hz, 1H), 3.13-3.23 (m, 1H), 2.96-3.06 (m, 1H), 2.65-2.76 (m, 1H), 2.14-2.23 (m, 1H). MS-ESI (m/z) calc'd for C₂₀H₁₅N₄O₂ [M+H]⁺: 343.1. Found 343.1.

### Example 38: 5-((3-(2-Morpholinopyridin-4-yl)-1H-indazol-5-yl)amino)-5,6,7,8-tetrahydronaphthalene-2-carbonitrile, enantiomer 1 and 2

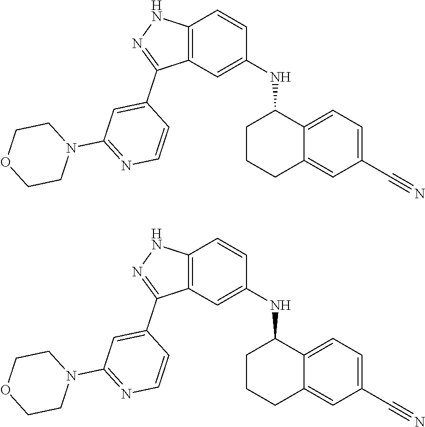 [SMILES: N#Cc1ccc2c(c1)CCC[C@@H]2Nc1ccc2[nH]nc(-c3ccnc(N4CCOCC4)c3)c2c1 ; N#Cc1ccc2c(c1)CCC[C@H]2Nc1ccc2[nH]nc(-c3ccnc(N4CCOCC4)c3)c2c1]

### Step 1: 5-((3-Bromo-1H-indazol-5-yl)amino)-5,6,7,8-tetrahydronaphthalene-2-carbonitrile

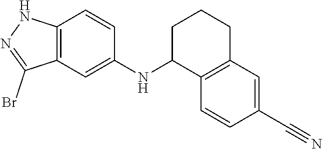 [SMILES: N#Cc1ccc2c(c1)CCCC2Nc1ccc2[nH]nc(Br)c2c1]

To a solution of 3-bromo-1H-indazol-5-amine (100 mg, 471.59 umol) and 1-oxotetralin-6-carbonitrile (40.37 mg, 235.80 umol) in toluene (5 mL) was added Ti(Oi-Pr)₄ (335.08 mg, 1.18 mmol) at 20° C. The mixture was stirred at 130° C. for 12 hrs. The mixture was concentrated and dissolved in MeOH (5 mL), then NaBH₄ (71.37 mg, 1.89 mmol) was added at 0° C. and the mixture was stirred at 20° C. for 4 hrs. The reaction mixture was quenched with saturated aqueous NH₄Cl solution at 20° C. and extracted with EtOAc (3×). The combined organic phases were concentrated to give a residue and the residue was purified by preparative-TLC (SiO₂, 100% EtOAc, Rf=0.60) to afford the title compound (45 mg, 52%) as a light brown gum. ¹H NMR (400 MHz, CDCl₃) δ 9.92 (br s, 1H), 7.56 (d, J=8.53 Hz, 1H), 7.43-7.47 (m, 2H), 7.32 (d, J=8.91 Hz, 1H), 6.88 (dd, J=8.91, 2.26 Hz, 1H), 6.74 (d, J=2.01 Hz, 1H), 4.70 (br d, J=4.77 Hz, 1H), 3.86 (br d, J=7.78 Hz, 1H), 2.78-2.97 (m, 2H), 2.07-2.15 (m, 1H), 1.87-2.03 (m, 3H). MS-ESI (m/z) calc'd for C₁₈H₁₆BrN₄ [M+H]⁺: 367.1/369.1. Found 367.0/369.0.

### Step 2: 5-((3-(2-Morpholinopyridin-4-yl)-1H-indazol-5-yl)amino)-5,6,7,8-tetrahydronaphthalene-2-carbonitrile

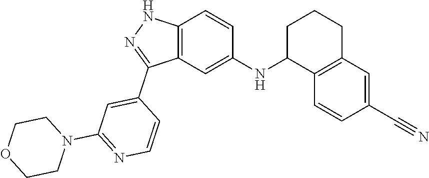 [SMILES: N#Cc1ccc2c(c1)CCCC2Nc1ccc2[nH]nc(-c3ccnc(N4CCOCC4)c3)c2c1]

To a solution of 5-((3-bromo-1H-indazol-5-yl)amino)-5,6,7,8-tetrahydronaphthalene-2-carbonitrile (80 mg, 217.84 umol) and (2-morpholino-4-pyridyl)boronic acid (54.38 mg, 261.41 umol) in H₂O (0.5 mL) and EtOH (2 mL) was added Pd(amphos)Cl₂ (15.42 mg, 21.79 umol) and AcOK (64.14 mg, 653.52 umol) under an N₂ atmosphere at 20° C. The mixture was stirred at 90° C. for 12 hrs. Another equivalent of (2-morpholino-4-pyridyl)boronic acid (54.38 mg, 261.41 umol) was added and Pd(amphos)Cl₂ (15.42 mg, 21.79 umol) under N₂ atmosphere at 20° C. The mixture was stirred at 90° C. for additional 12 hrs. The reaction mixture was concentrated. The residue was diluted with H₂O and extracted with EtOAc (3×). The combined organic layers were concentrated and the residue was purified by preparative-HPLC using Method AY to afford the title compound (12 mg, 12%) as a yellow solid. MS-ESI (m/z) calc'd for C₂₇H₂₇N₆O [M+H]⁺: 451.2. Found 451.2.

### Step 3: 5-((3-(2-Morpholinopyridin-4-yl)-1H-indazol-5-yl)amino)-5,6,7,8-tetrahydronaphthalene-2-carbonitrile, enantiomer 1 and 2

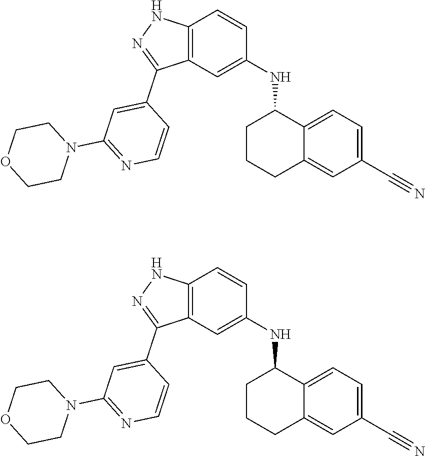 [SMILES: N#Cc1ccc2c(c1)CCC[C@@H]2Nc1ccc2[nH]nc(-c3ccnc(N4CCOCC4)c3)c2c1 ; N#Cc1ccc2c(c1)CCC[C@H]2Nc1ccc2[nH]nc(-c3ccnc(N4CCOCC4)c3)c2c1]

5-((3-(2-Morpholinopyridin-4-yl)-1H-indazol-5-yl)amino)-5,6,7,8-tetrahydronaphthalene-2-carbonitrile was subjected to chiral separation using Method AZ to afford 5-((3-(2-morpholinopyridin-4-yl)-1H-indazol-5-yl)amino)-5,6,7,8-tetrahydronaphthalene-2-carbonitrile, enantiomer 1 (2.77 mg, 30%) as a yellow solid. ¹H NMR (400 MHz, MeOH-d₄) δ 8.16 (d, J=5.50 Hz, 1H), 7.61 (d, J=8.07 Hz, 1H), 7.53 (s, 1H), 7.47 (d, J=7.95 Hz, 1H), 7.42 (d, J=8.80 Hz, 1H), 7.30 (s, 1H), 7.27 (d, J=5.38 Hz, 1H), 7.10 (s, 1H), 7.06 (dd, J=8.93, 2.08 Hz, 1H), 4.71 (t, J=5.87 Hz, 1H), 3.79-3.85 (m, 4H), 3.48-3.54 (m, 4H), 2.80-2.95 (m, 2H), 1.87-2.14 (m, 4H). MS-ESI (m/z) calc'd for C₂₇H₂₇N₆O [M+H]⁺: 451.2. Found 451.2. A later eluting fraction was also isolated to afford 5-((3-(2-morpholinopyridin-4-yl)-1H-indazol-5-yl)amino)-5,6,7,8-tetrahydronaphthalene-2-carbonitrile, enantiomer 2 (2.82 mg, 31%) as a yellow solid. ¹H NMR (400 MHz, MeOH-d₄) δ 8.18 (d, J=5.26 Hz, 1H), 7.61 (d, J=8.07 Hz, 1H), 7.53 (s, 1H), 7.47 (d, J=8.19 Hz, 1H), 7.41 (d, J=8.93 Hz, 1H), 7.26 (s, 1H), 7.23 (d, J=5.26 Hz, 1H), 7.10 (s, 1H), 7.05 (dd, J=8.93, 2.08 Hz, 1H), 4.71 (t, J=5.87 Hz, 1H), 3.78-3.84 (m, 4H), 3.47-3.53 (m, 4H), 2.80-2.97 (m, 2H), 1.88-2.13 (m, 4H). MS-ESI (m/z) calc'd for C₂₇H₂₇N₆O [M+H]⁺: 451.2. Found 451.1.

### Example 39: 5-((3-(2-Morpholinopyridin-4-yl)-1H-indazol-5-yl)oxy)-5,6,7,8-tetrahydronaphthalene-2-carbonitrile, enantiomer 1 and 2

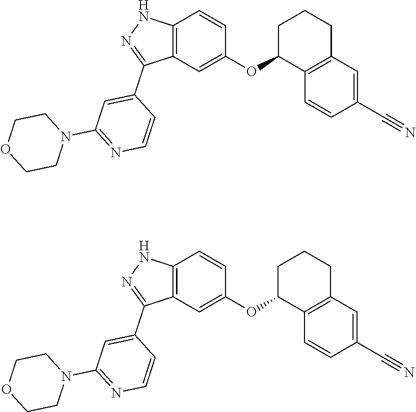 [SMILES: N#Cc1ccc2c(c1)CCC[C@@H]2Oc1ccc2[nH]nc(-c3ccnc(N4CCOCC4)c3)c2c1 ; N#Cc1ccc2c(c1)CCC[C@H]2Oc1ccc2[nH]nc(-c3ccnc(N4CCOCC4)c3)c2c1]

### Step 1: 4-(4-bromopyridin-2-yl)morpholine

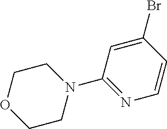 [SMILES: Brc1ccnc(N2CCOCC2)c1]

To a solution of 4-bromo-2-fluoro-pyridine (2 g, 11.36 mmol) in DMF (10 mL) was added morpholine (1.19 g, 13.64 mmol, 1.20 mL) and Cs₂CO₃ (7.41 g, 22.73 mmol) at 20° C. The mixture was stirred at 100° C. for 12 hrs. The reaction mixture was then diluted with H₂O and extracted with EtOAc (3×), the combined organic layers were dried over Na₂SO₄ and evaporated to give a residue. The residue was purified by flash silica gel chromatography (ISCO; 12 g SepaFlash column) using a 0-8% EtOAc/petroleum ether gradient eluent to afford the title compound (2.13 g, 75%) as a white solid. MS-ESI (m/z) calc'd for C₉H₁₂BrN₂O [M+H]⁺: 243.0/245.0. Found 243.0/245.0.

### Step 2: (2-Morpholinopyridin-4-yl)boronic acid

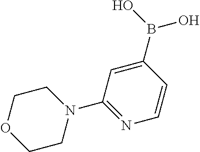 [SMILES: OB(O)c1ccnc(N2CCOCC2)c1]

A mixture of 4-(4-bromopyridin-2-yl)morpholine (1.08 g, 4.44 mmol), bis(pinacolato)diboron (1.35 g, 5.33 mmol), AcOK (1.09 g, 11.11 mmol), [1,1-bis(diphenylphosphino)ferrocene]dichloropalladium(II) (325.07 mg, 444.26 umol) in 1,4-dioxane (15 mL) was degassed and purged with N₂ (3×) at 20° C., and then the mixture was stirred at 80° C. for 12 hrs under N₂ atmosphere. The reaction mixture was filtered and the filtrate was evaporated to afford the title compound (2.1 g, 90%) as a black oil. MS-ESI (m/z) calc'd for C₉H₁₄BN₂O₃ [M+H]⁺: 209.1. Found 209.0.

### Step 3: 4-(4-(5-Bromo-1H-indazol-3-yl)pyridin-2-yl)morpholine

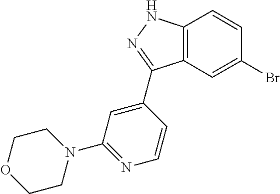 [SMILES: Brc1ccc2[nH]nc(-c3ccnc(N4CCOCC4)c3)c2c1]

A mixture of (2-morpholinopyridin-4-yl)boronic acid (1.29 g, 6.19 mmol), 5-bromo-3-iodo-1H-indazole (1 g, 3.10 mmol), AcOK (911.74 mg, 9.29 mmol), bis(4-(di-tert-butylphosphanyl)-N,N-dimethyl-aniline); dichloropalladium (219.27 mg, 309.67 umol) in EtOH (20 mL) and H₂O (4 mL) was degassed and purged with N₂ (3×) at 15° C., and then the mixture was stirred at 90° C. for 12 hrs under N₂ atmosphere. The reaction mixture was concentrated under reduced pressure to remove solvent. The residue was purified by flash silica gel chromatography (ISCO; 12 g SepaFlash column) using a 0-20% EtOAc/petroleum ether gradient eluent to afford the title compound (580 mg, 26%) as a brown solid. MS-ESI (m/z) calc'd for C₁₆H₁₆BrN₄O [M+H]⁺: 359.0/361.0. Found 359.0/361.0.

### Step 4: 4-(4-(5-Bromo-1-(tetrahydro-2H-pyran-2-yl)-1H-indazol-3-yl)pyridin-2-yl)morpholine

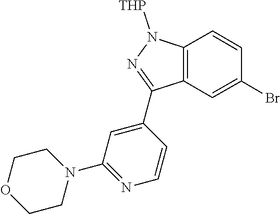

To a solution of 4-(4-(5-bromo-1H-indazol-3-yl)pyridin-2-yl)morpholine (580 mg, 1.61 mmol) and 3,4-dihydro-2H-pyran-2-ylmethanol (407.44 mg, 4.84 mmol, 442.87 uL) in CHCl₃ (7 mL) was added methanesulfonic acid (15.52 mg, 161.46 umol) at 15° C. The mixture was stirred at 70° C. for 12 hrs. The reaction mixture was concentrated to give a residue and the residue was purified by preparative-TLC (SiO₂, petroleum ether/EtOAc=1:1, Rf=0.60) to afford the title compound (470 mg, 66%) as a white solid. MS-ESI (m/z) calc'd for C₂₁H₂₄BrN₄O₂ [M+H]⁺: 443.1/445.1. Found 443.1/445.2.

### Step 5: 4-(4-(1-(Tetrahydro-2H-pyran-2-yl)-5-(4,4,5,5-tetramethyl-1,3,2-dioxaborolan-2-yl)-1H-indazol-3-yl)pyridin-2-yl)morpholine

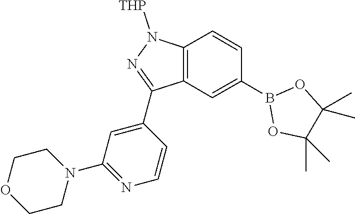

To a solution of 4-(4-(5-bromo-1-(tetrahydro-2H-pyran-2-yl)-1H-indazol-3-yl)pyridin-2-yl)morpholine (470 mg, 1.06 mmol) in 1,4-dioxane (5 mL) was added 4,4,5,5-tetramethyl-2-(4,4,5,5-tetramethyl-1,3,2-dioxaborolan-2-yl)-1,3,2-dioxaborolane (403.82 mg, 1.59 mmol), [1,1-bis(diphenylphosphino)ferrocene]dichloropalladium(II) (77.57 mg, 106.01 umol), and KOAc (312.13 mg, 3.18 mmol) at 20° C. The mixture was stirred at 100° C. for 2 hrs and then diluted with H₂O and extracted with EtOAc (3×). The combined organic layers were dried over Na₂SO₄ and evaporated to give a residue. The residue was purified by flash silica gel chromatography (ISCO; 4 g SepaFlash column) using a 0-29% EtOAc/petroleum ether gradient eluent to afford the title compound (480 mg, 92%) as a yellow oil. MS-ESI (m/z) calc'd for C₂₇H₃₆BN₄O₄ [M+H]⁺: 491.3. Found 491.3.

### Step 6: 3-(2-Morpholinopyridin-4-yl)-1-(tetrahydro-2H-pyran-2-yl)-1H-indazol-5-ol

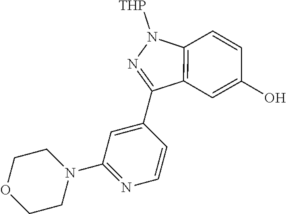

A mixture of 4-(4-(1-(tetrahydro-2H-pyran-2-yl)-5-(4,4,5,5-tetramethyl-1,3,2-dioxaborolan-2-yl)-1H-indazol-3-yl)pyridin-2-yl)morpholine (480 mg, 978.79 umol), sodium perborate tetrahydrate (451.79 mg, 2.94 mmol) in THF (3 mL) and H₂O (3 mL) was degassed and purged with N₂ (3×) at 15° C., and then the mixture was stirred at 50° C. for 1 hr under N₂ atmosphere. The reaction mixture was concentrated under reduced pressure to remove solvent. The residue was purified by flash silica gel chromatography (ISCO; 12 g SepaFlash column) using a 0-12% EtOAc/petroleum ether gradient eluent to afford the title compound (270 mg, 72%) as a brown oil. MS-ESI (m/z) calc'd for C₂₁H₂₅N₄O₃ [M+H]⁺: 381.2. Found 381.2.

### Step 7: 5-((3-(2-Morpholinopyridin-4-yl)-1-(tetrahydro-2H-pyran-2-yl)-1H-indazol-5-yl)oxy)-5,6,7,8-tetrahydronaphthalene-2-carbonitrile

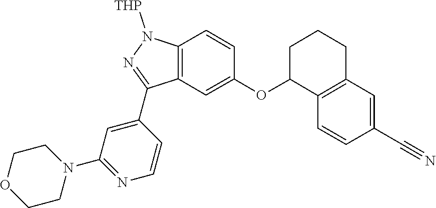

To a solution of 3-(2-morpholinopyridin-4-yl)-1-(tetrahydro-2H-pyran-2-yl)-1H-indazol-5-ol (200 mg, 525.71 umol) in THF (5 mL) was added tributylphosphine (319.08 mg, 1.58 mmol, 389.12 uL) and N-(piperidine-1-carbonylimino)piperidine-1-carboxamide (397.93 mg, 1.58 mmol) and 1-hydroxytetralin-6-carbonitrile (91.06 mg, 525.71 umol) at 0° C. The mixture was stirred at 45° C. for 1 hr. The reaction mixture was concentrated under reduced pressure to give a residue and the residue was purified by flash silica gel chromatography (ISCO; 4 g SepaFlash column) using a 0-30% EtOAc/petroleum ether gradient eluent to afford the title compound (175 mg, 62%) as a yellow solid. MS-ESI (m/z) calc'd for C₃₂H₃₄N₅O₃ [M+H]⁺: 536.3. Found 536.2.

### Step 8: 5-((3-(2-Morpholinopyridin-4-yl)-1H-indazol-5-yl)oxy)-5,6,7,8-tetrahydronaphthalene-2-carbonitrile

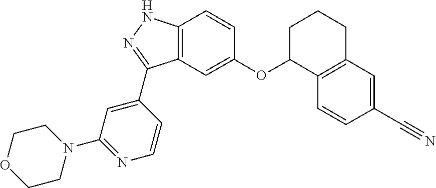 [SMILES: N#Cc1ccc2c(c1)CCCC2Oc1ccc2[nH]nc(-c3ccnc(N4CCOCC4)c3)c2c1]

To a solution of 5-((3-(2-morpholinopyridin-4-yl)-1-(tetrahydro-2H-pyran-2-yl)-1H-indazol-5-yl)oxy)-5,6,7,8-tetrahydronaphthalene-2-carbonitrile (155 mg, 289.38 umol) in MeOH (5 mL) and H₂O (1 mL) was added p-toluenesulfonic acid (249.16 mg, 1.45 mmol) at 20° C. The mixture was stirred at 70° C. for 3 hrs. The reaction mixture was concentrated under reduced pressure to give a residue. The residue was purified by preparative-HPLC using Method BB to afford the title compound (40 mg, 24%) as a yellow solid. MS-ESI (m/z) calc'd for C₂₇H₂₆N₅O₂ [M+H]⁺: 452.2. Found 452.2.

### Step 9: 5-((3-(2-Morpholinopyridin-4-yl)-1H-indazol-5-yl)oxy)-5,6,7,8-tetrahydronaphthalene-2-carbonitrile, enantiomer 1 and 2

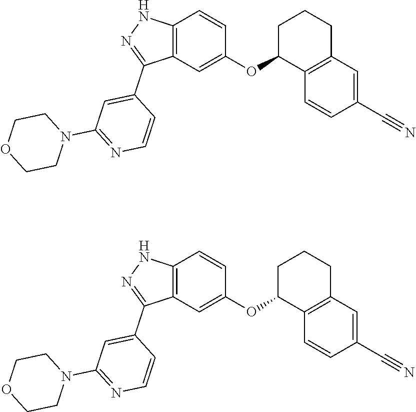 [SMILES: N#Cc1ccc2c(c1)CCC[C@@H]2Oc1ccc2[nH]nc(-c3ccnc(N4CCOCC4)c3)c2c1 ; N#Cc1ccc2c(c1)CCC[C@H]2Oc1ccc2[nH]nc(-c3ccnc(N4CCOCC4)c3)c2c1]

5-((3-(2-Morpholinopyridin-4-yl)-1H-indazol-5-yl)oxy)-5,6,7,8-tetrahydronaphthalene-2-carbonitrile was subjected to chiral separation using Method BC to afford 5-((3-(2-morpholinopyridin-4-yl)-1H-indazol-5-yl)oxy)-5,6,7,8-tetrahydronaphthalene-2-carbonitrile, enantiomer 1 (1.60 mg, 18%). ¹H NMR (400 MHz, DMSO-d₆) δ 13.33 (s, 1H), 8.24 (d, J=5.29 Hz, 1H), 7.69 (s, 1H), 7.62-7.66 (m, 2H), 7.56-7.60 (m, 2H), 7.28-7.30 (m, 1H), 7.26 (s, 1H), 7.20 (dd, J=8.82, 2.20 Hz, 1H), 5.65 (t, J=4.74 Hz, 1H), 3.71-3.75 (m, 4H), 3.48-3.53 (m, 4H), 2.76-2.93 (m, 2H), 2.00-2.07 (m, 2H), 1.77-1.94 (m, 2H). MS-ESI (m/z) calc'd for C₂₇H₂₆N₅O₂ [M+H]⁺: 452.2. Found 452.2. A later eluting fraction was also isolated to afford 5-((3-(2-morpholinopyridin-4-yl)-1H-indazol-5-yl)oxy)-5,6,7,8-tetrahydronaphthalene-2-carbonitrile, enantiomer 2 (3.00 mg, 33%). ¹H NMR (400 MHz, DMSO-d₆) δ 13.39 (s, 1H), 8.24 (d, J=5.29 Hz, 1H), 7.69 (s, 1H), 7.63-7.66 (m, 2H), 7.55-7.60 (m, 2H), 7.28-7.30 (m, 1H), 7.26 (s, 1H), 7.20 (dd, J=9.04, 1.98 Hz, 1H), 5.65 (t, J=5.07 Hz, 1H), 3.71-3.75 (m, 4H), 3.49-3.52 (m, 4H), 2.74-2.94 (m, 2H), 2.00-2.06 (m, 2H), 1.75

### Example 40: 1-((3-(2-Methoxypyridin-4-yl)-1H-indazol-5-yl)amino)-2,3-dihydro-1H-indene-5-carbonitrile

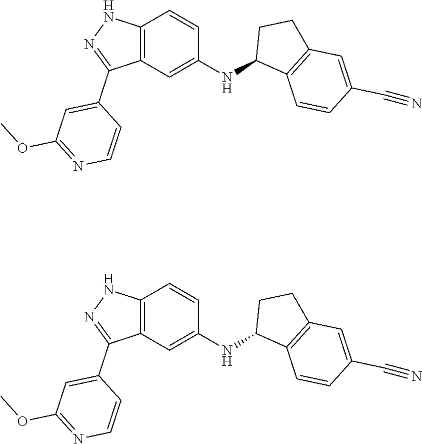 [SMILES: COc1cc(-c2n[nH]c3ccc(N[C@H]4CCc5cc(C#N)ccc54)cc23)ccn1 ; COc1cc(-c2n[nH]c3ccc(N[C@@H]4CCc5cc(C#N)ccc54)cc23)ccn1]

### Step 1: 1-Oxo-2,3-dihydro-1H-indene-5-carbonitrile

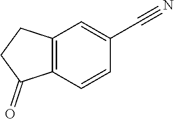 [SMILES: N#Cc1ccc2c(c1)CCC2=O]

A mixture of 5-bromo-2,3-dihydro-1H-inden-1-one (1 g, 4.74 mmol), CuCN (551.67 mg, 6.16 mmol) taken up into a microwave tube in NMP (4 mL) at 20° C. And then the mixture was stirred at 190° C. for 1 hr under an N₂ atmosphere using microwave irradiation. The reaction mixture was filtered and the filtrate was diluted with H₂O and extracted with EtOAc (3×). The combined organic layers were dried over Na₂SO₄ and evaporated to give a residue. The residue was purified by flash silica gel chromatography (ISCO; 20 g SepaFlash column) using a 0-17% EtOAc/petroleum ether gradient eluent to afford the title compound (820 mg, 82%) as a yellow solid. MS-ESI (m/z) calc'd for C₁₀H₈NO [M+H]⁺: 158.1. Found 158.0.

### Step 2: 3-Bromo-1H-indazol-5-amine

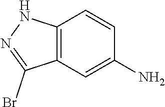 [SMILES: Nc1ccc2[nH]nc(Br)c2c1]

To a solution of 3-bromo-5-nitro-1H-indazole (1 g, 4.13 mmol) in EtOH (10 mL) was added SnCl₂.2H₂O (3.73 g, 16.53 mmol) at 20° C. The mixture was stirred at 90° C. for 12 hrs. The reaction mixture was adjusted to pH=8 with 2 N aqueous NaOH and filtered. The solid was washed with EtOH and the filtrate was evaporated to give a residue. The residue was diluted with H₂O and extracted with EtOAc (3×). This procedure was conducted an additional time and the organic extracts were combined, washed with brine, dried over anhydrous Na₂SO₄ and evaporated to dryness to afford the title compound (1.1 g, 60%) as a black solid. MS-ESI (m/z) calc'd for C₇H₇BrN₃ [M+H]⁺: 212.0/214.0. Found 212.0/214.0.

### Step 3: 3-(2-Methoxypyridin-4-yl)-1H-indazol-5-amine

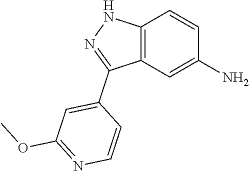 [SMILES: COc1cc(-c2n[nH]c3ccc(N)cc23)ccn1]

A mixture of 3-bromo-1H-indazol-5-amine (200 mg, 943.19 umol), (2-methoxy-4-pyridyl)boronic acid (173.11 mg, 1.13 mmol), AcOK (277.70 mg, 2.83 mmol), bis(4-(di-tert-butylphosphanyl)-N,N-dimethylaniline); dichloropalladium (133.57 mg, 188.64 umol) in H₂O (2 mL) and EtOH (8 mL) was degassed and purged with N₂ (3×) at 20° C., and then the mixture was stirred at 90° C. for 12 hrs under an N₂ atmosphere. The reaction mixture was evaporated to remove EtOH, then it was diluted with H₂O and extracted with EtOAc (3×), the combined organic layers were dried over Na₂SO₄ and evaporated to give a residue. The residue was purified by preparative-TLC (SiO₂, petroleum ether/EtOAc=1:1, Rf=0.20) to afford the title compound (126 mg, 56%) as a yellow solid. MS-ESI (m/z) calc'd for C₁₃H₁₃N₄O [M+H]⁺: 241.1. Found 241.1.

### Step 4: 1-((3-(2-Methoxypyridin-4-yl)-1H-indazol-5-yl)amino)-2,3-dihydro-1H-indene-5-carbonitrile

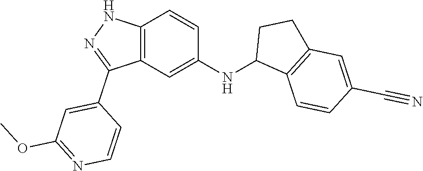 [SMILES: COc1cc(-c2n[nH]c3ccc(NC4CCc5cc(C#N)ccc54)cc23)ccn1]

To a solution of 3-(2-methoxypyridin-4-yl)-1H-indazol-5-amine (100.41 mg, 417.93 umol), 1-oxoindane-5-carbonitrile (54.74 mg, 348.28 umol) in MeOH (2 mL) and 1,2-dichloroethane (2 mL) was added AcOH (2.09 mg, 34.83 umol) to make pH=5 at 20° C., the mixture was stirred at 70° C. for 5 hrs. Then NaBH₃CN (65.66 mg, 1.04 mmol) was added and the reaction mixture was stirred at 70° C. for 12 hrs. The reaction mixture was concentrated to give a residue that was purified by preparative-HPLC using Method BD to afford the title compound (25.5 mg, 15%) as yellow oil. MS-ESI (m/z) calc'd for C₂₃H₂₀N₅O [M+H]⁺: 382.2. Found 382.2.

### Step 5: 1-((3-(2-Methoxypyridin-4-yl)-1H-indazol-5-yl)amino)-2,3-dihydro-1H-indene-5-carbonitrile, enantiomer 1 and 2

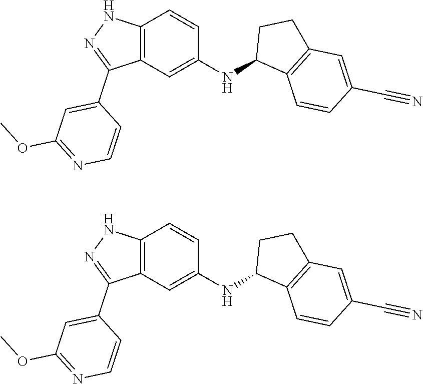 [SMILES: COc1cc(-c2n[nH]c3ccc(N[C@H]4CCc5cc(C#N)ccc54)cc23)ccn1 ; COc1cc(-c2n[nH]c3ccc(N[C@@H]4CCc5cc(C#N)ccc54)cc23)ccn1]

1-((3-(2-Methoxypyridin-4-yl)-1H-indazol-5-yl)amino)-2,3-dihydro-1H-indene-5-carbonitrile was subjected to chiral separation using Method BE to afford 1-((3-(2-methoxypyridin-4-yl)-1H-indazol-5-yl)amino)-2,3-dihydro-1H-indene-5-carbonitrile, enantiomer 1 (3.12 mg, 35%). ¹H NMR (400 MHz, MeOD-d₄) δ 8.07 (d, J=5.38 Hz, 1H), 7.53 (s, 1H), 7.40-7.45 (m, 2H), 7.38 (dd, J=5.50, 1.22 Hz, 1H), 7.32 (d, J=8.93 Hz, 1H), 7.12 (s, 1H), 7.04 (s, 1H), 6.96 (dd, J=8.99, 2.02 Hz, 1H), 5.09 (t, J=7.58 Hz, 1H), 3.86 (s, 3H), 2.83-3.02 (m, 2H), 2.50-2.60 (m, 1H), 1.94 (dq, J=12.72, 8.48 Hz, 1H). MS-ESI (m/z) calc'd for C₂₃H₂₀N₅O [M+H]⁺: 382.1. Found 382.1. A later eluting fraction was also isolated to afford 1-((3-(2-methoxypyridin-4-yl)-1H-indazol-5-yl)amino)-2,3-dihydro-1H-indene-5-carbonitrile, enantiomer 2 (3.89 mg, 43%). ¹H NMR (400 MHz, MeOD-d₄) δ 8.07 (d, J=5.50 Hz, 1H), 7.53 (s, 1H), 7.40-7.46 (m, 2H), 7.37-7.40 (m, 1H), 7.32 (d, J=8.93 Hz, 1H), 7.12 (s, 1H), 7.04 (s, 1H), 6.96 (dd, J=8.99, 2.02 Hz, 1H), 5.09 (t, J=7.58 Hz, 1H), 3.86 (s, 3H), 2.82-3.04 (m, 2H), 2.49-2.62 (m, 1H), 1.94 (dq, J=12.72, 8.48 Hz, 1H). MS-ESI (m/z) calc'd for C₂₃H₂₀N₅O [M+H]⁺: 382.1. Found 382.1.

### Example 41: 7-((3-(Oxazol-5-yl)-1H-indazol-5-yl)oxy)-6,7-dihydro-5H-cyclopenta[c]pyridazine-3-carbonitrile

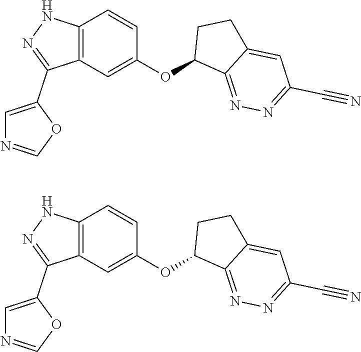 [SMILES: N#Cc1cc2c(nn1)[C@@H](Oc1ccc3[nH]nc(-c4cnco4)c3c1)CC2 ; N#Cc1cc2c(nn1)[C@H](Oc1ccc3[nH]nc(-c4cnco4)c3c1)CC2]

### Step 1: 2,4,4a,5,6,7-Hexahydro-3H-cyclopenta[c]pyridazin-3-one

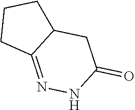

To a solution of ethyl 2-(2-oxocyclopentyl) acetate (5 g, 29.38 mmol) in EtOH (60 mL) was added NH₂NH₂.H₂O (1.65 g, 32.31 mmol) at 20° C. The mixture was stirred at 80° C. for 12 hrs. The reaction mixture was concentrated to afford the title compound (4 g, 98%) as a light yellow solid. MS-ESI (m/z) calc'd for C₇H₁₁N₂O [M+H]⁺: 139.1. Found 139.2.

### Step 2: 2,5,6,7-Tetrahydro-3H-cyclopenta[c]pyridazin-3-one

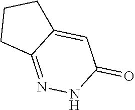

To a solution of 2,4,4a,5,6,7-hexahydro-3H-cyclopenta[c]pyridazin-3-one (4 g, 28.95 mmol) in ACN (120 mL) was added CuCl₂ (7.78 g, 57.90 mmol) at 20° C. The mixture was stirred at 80° C. 1 hr. The reaction mixture was cooled to 20° C., filtered, the filtrate was concentrated to afford the title compound (3.9 g, 98%) as a dark green solid. MS-ESI (m/z) calc'd for C₇H₉N₂O [M+H]⁺: 137.1. Found 137.2.

### Step 3: 3-Chloro-6,7-dihydro-5H-cyclopenta[c]pyridazine

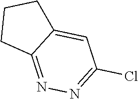

A solution of 2,5,6,7-tetrahydro-3H-cyclopenta[c]pyridazin-3-one (1.16 g, 8.52 mmol) in POCl₃ (10 mL) was stirred at 90° C. for 1 hr. The reaction mixture was quenched by addition Na₂CO₃ saturated solution at 0° C. to pH=7, and then diluted with H₂O and extracted with EtOAc (3×). The combined organic phase was dried with anhydrous Na₂SO₄, the mixture was filtered and the filtrate was concentrated to give a residue. This procedure was conducted 2 additional times and the extracts were combined and purified by flash silica gel chromatography (ISCO; 25 g SepaFlash column) using a 0-20% EtOAc/petroleum ether gradient eluent to afford the title compound (2.82 g, 71%) as a white solid. MS-ESI (m/z) calc'd for C₇H₈ClN₂ [M+H]⁺: 155.0/157.0. Found 155.0/157.0.

### Step 4: 3-Chloro-6,7-dihydro-5H-cyclopenta[c]pyridazine 1-oxide

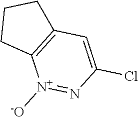

To a solution of 3-chloro-6,7-dihydro-5H-cyclopenta[c]pyridazine (1 g, 6.47 mmol) in CH₂Cl₂ (30 mL) was added MCPBA (1.31 g, 6.47 mmol) at 20° C. and the mixture was stirred for 12 hrs. Then a 10% aqueous solution of Na₂SO₃ was added and stirring continued at 0° C. for 1 hr. The mixture was then diluted with H₂O and extracted with CH₂Cl₂ (3×). The combined organic phases were dried with anhydrous Na₂SO₄, filtered and the filtrate was concentrated to afford the title compound (1 g, 90%) as a white solid. MS-ESI (m/z) calc'd for C₇H₈ClN₂O [M+H]⁺: 171.0/173.0. Found 171.0/173.0.

### Step 5: 3-Chloro-6,7-dihydro-5H-cyclopenta[c]pyridazin-7-yl 2,2,2-trifluoroacetate

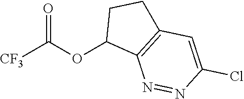 [SMILES: O=C(OC1CCc2cc(Cl)nnc21)C(F)(F)F]

A solution of 3-chloro-6,7-dihydro-5H-cyclopenta[c]pyridazine 1-oxide (1 g, 5.86 mmol) in TFAA (8.62 g, 41.03 mmol) was stirred at 50° C. for 5 hrs. The mixture was then concentrated to afford the title compound (1.3 g, 83%) as a black gum which was used without further purification. MS-ESI (m/z) calc'd for C₉H₇ClF₃N₂O₂ [M+H]⁺: 267.0/269.0. Found 267.0/269.0.

### Step 6: 3-Chloro-6,7-dihydro-5H-cyclopenta[c]pyridazin-7-ol

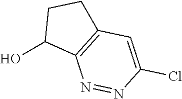 [SMILES: OC1CCc2cc(Cl)nnc21]

To a solution of NaOH (450.11 mg, 11.25 mmol) in H₂O (30 mL) was added 3-chloro-6,7-dihydro-5H-cyclopenta[c]pyridazin-7-yl 2,2,2-trifluoroacetate (1 g, 3.75 mmol) at 20° C. and the mixture was stirred for 1 hr. The reaction mixture was diluted with H₂O and extracted with EtOAc (6×). The combined organic phases were dried with anhydrous Na₂SO₄, filtered and the filtrate was concentrated. The residue was purified by flash silica gel chromatography (ISCO; 4 g SepaFlash column) using a 0-35% EtOAc/petroleum ether gradient eluent to afford the title compound (382 mg, 60%) as a yellow solid. ¹H NMR (400 MHz, DMSO-d₆) δ 7.79-7.81 (m, 1H), 5.83 (br s, 1H), 5.17 (dd, J=7.00, 5.50 Hz, 1H), 2.95-3.10 (m, 1H), 2.75-2.90 (m, 1H), 2.30-2.45 (m, 1H), 1.79-1.99 (m, 1H). MS-ESI (m/z) calc'd for C₇H₈ClN₂O [M+H]⁺: 171.0/173.0. Found 171.1/173.1.

### Step 7: 5-(5-((3-Chloro-6,7-dihydro-5H-cyclopenta[c]pyridazin-7-yl)oxy)-1-(tetrahydro-2H-pyran-2-yl)-1H-indazol-3-yl)oxazole

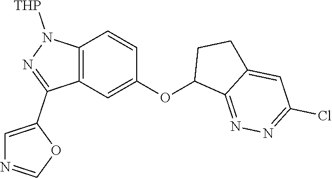

To a solution of 3-chloro-6,7-dihydro-5H-cyclopenta[c]pyridazin-7-ol (143.51 mg, 841.23 umol) in toluene (5 mL) was added 3-oxazol-5-yl-1-tetrahydropyran-2-yl-indazol-5-ol (120 mg, 420.61 umol), 1,1′-(azodicarbonyl)dipiperidine (212.25 mg, 841.23 umol) and tributylphosphine (170.19 mg, 841.23 umol) at 0° C. The mixture was stirred at 90° C. for 12 hrs. The mixture was concentrated and purified by preparative-TLC (SiO₂, 100% EtOAc, Rf=0.30) to afford the title compound (100 mg, 54%) as a brown solid. MS-ESI (m/z) calc'd for C₂₂H₂₁ClN₅O₃ [M+H]⁺: 438.1/440.1. Found 438.3/440.2.

### Step 8: 7-((3-(Oxazol-5-yl)-1-(tetrahydro-2H-pyran-2-yl)-1H-indazol-5-yl)oxy)-6,7-dihydro-5H-cyclopenta[c]pyridazine-3-carbonitrile

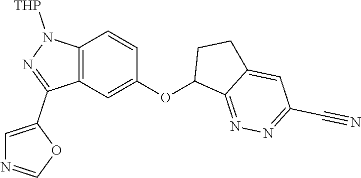

A mixture of Zn(CN)₂ (75.09 mg, 639.45 umol), 5-[5-[(3-chloro-6,7-dihydro-5H-cyclopenta[c]pyridazin-7-yl)oxy]-1-tetrahydropyran-2-yl-indazol-3-yl]oxazole (140 mg, 319.72 umol), Zn (20.91 mg, 319.72 umol), 1,1-bis(diphenylphosphino)ferrocene (5.32 mg, 9.59 umol) and Pd₂dba₃(0) (17.57 mg, 19.18 umol) in DMA (5 mL) was degassed and purged with N₂ (3×) at 20° C., and then the mixture was stirred at 90° C. for 2 hrs under N₂ atmosphere. The reaction was filtered and the filtrate was evaporated to give a residue. The residue was purified by preparative-TLC (SiO₂, petroleum ether/EtOAc=1:2, Rf=0.27) to afford the title compound (90 mg, 66%) as a brown solid. MS-ESI (m/z) calc'd for C₂₃H₂₁N₆O₃ [M+H]⁺: 429.2. Found 429.1.

### Step 9: 7-((3-(Oxazol-5-yl)-1H-indazol-5-yl)oxy)-6,7-dihydro-5H-cyclopenta[c]pyridazine-3-carbonitrile

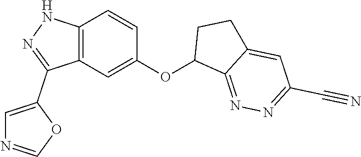 [SMILES: N#Cc1cc2c(nn1)C(Oc1ccc3[nH]nc(-c4cnco4)c3c1)CC2]

A solution of 7-((3-(oxazol-5-yl)-1-(tetrahydro-2H-pyran-2-yl)-1H-indazol-5-yl)oxy)-6,7-dihydro-5H-cyclopenta[c]pyridazine-3-carbonitrile (80 mg, 186.72 umol) in TFA (0.8 mL) and CH₂Cl₂ (3.2 mL) was stirred at 20° C. for 1.5 hrs. The reaction mixture was diluted with an aqueous NaHCO₃ solution to pH=8. The layers were separated and the aqueous layer was extracted with EtOAc (3×). The combined organic layers were dried over Na₂SO₄, filtered and concentrated under reduced pressure to give a residue. The residue was purified by preparative-HPLC using Method CK to afford the title compound (27.8 mg, 66%) as a pale green solid. MS-ESI (m/z) calc'd for C₁₈H₁₃N₆O₂ [M+H]⁺: 345.1. Found 345.2.

### Step 10: 7-((3-(Oxazol-5-yl)-1H-indazol-5-yl)oxy)-6,7-dihydro-5H-cyclopenta[c]pyridazine-3-carbonitrile, enantiomer 1 and 2

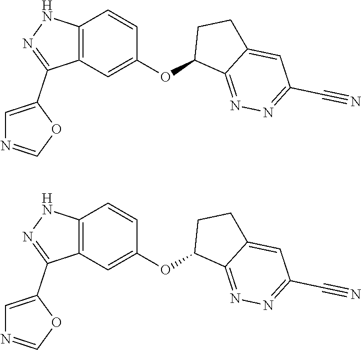 [SMILES: N#Cc1cc2c(nn1)[C@@H](Oc1ccc3[nH]nc(-c4cnco4)c3c1)CC2 ; N#Cc1cc2c(nn1)[C@H](Oc1ccc3[nH]nc(-c4cnco4)c3c1)CC2]

rac-7-((3-(Oxazol-5-yl)-1H-indazol-5-yl)oxy)-6,7-dihydro-5H-cyclopenta[c]pyridazine-3-carbonitrile was subjected to chiral separation using Method BF to afford 7-((3-(oxazol-5-yl)-1H-indazol-5-yl)oxy)-6,7-dihydro-5H-cyclopenta[c]pyridazine-3-carbonitrile, enantiomer 1 (2.41 mg, 27%). ¹H NMR (400 MHz, DMSO-d₆) δ 13.45 (br s, 1H), 8.53 (s, 1H), 8.42 (s, 1H), 7.84 (s, 1H), 7.76 (d, J=1.83 Hz, 1H), 7.58 (d, J=9.05 Hz, 1H), 7.22 (dd, J=8.99, 2.14 Hz, 1H), 6.30 (dd, J=6.91, 4.59 Hz, 1H), 3.02-3.30 (m, 2H), 2.73 (td, J=14.27, 6.79 Hz, 1H), 2.21-2.31 (m, 1H). MS-ESI (m/z) calc'd for C₁₈H₁₃N₆O₂ [M+H]⁺: 345.1. Found 345.0. A later eluting fraction was also isolated to afford 7-((3-(oxazol-5-yl)-1H-indazol-5-yl)oxy)-6,7-dihydro-5H-cyclopenta[c]pyridazine-3-carbonitrile, enantiomer 2 (2.79 mg, 31%). ¹H NMR (400 MHz, DMSO-d₆) δ 13.45 (br s, 1H), 8.53 (s, 1H), 8.42 (s, 1H), 7.84 (s, 1H), 7.76 (d, J=1.83 Hz, 1H), 7.58 (d, J=9.05 Hz, 1H), 7.22 (dd, J=9.05, 2.20 Hz, 1H), 6.30 (dd, J=6.97, 4.65 Hz, 1H), 3.04-3.29 (m, 2H), 2.73 (td, J=14.18, 6.97 Hz, 1H), 2.21-2.32 (m, 1H). MS-ESI (m/z) calc'd for C₁₈H₁₃N₆O₂ [M+H]⁺: 345.1. Found 345.0.

### Example 42: 2,2-Difluoro-1-((3-(oxazol-5-yl)-1H-indazol-5-yl)amino)-2,3-dihydro-1H-indene-5-carbonitrile, enantiomer 1 and 2

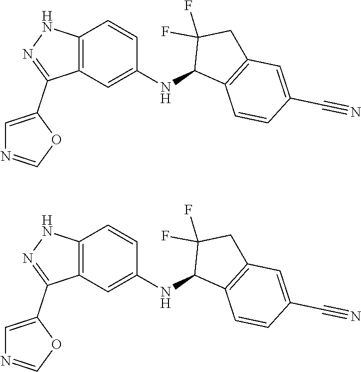 [SMILES: N#Cc1ccc2c(c1)CC(F)(F)[C@@H]2Nc1ccc2[nH]nc(-c3cnco3)c2c1 ; N#Cc1ccc2c(c1)CC(F)(F)[C@@H]2Nc1ccc2[nH]nc(-c3cnco3)c2c1]

### Step 1: 2,2-Difluoro-1-((3-(oxazol-5-yl)-JH-indazol-5-yl)amino)-2,3-dihydro-JH-indene-5-carbonitrile

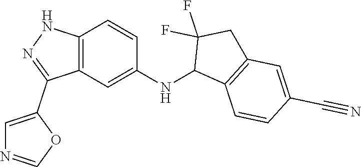 [SMILES: N#Cc1ccc2c(c1)CC(F)(F)C2Nc1ccc2[nH]nc(-c3cnco3)c2c1]

A mixture of N-(5-bromo-2,2-difluoro-indan-1-yl)-3-oxazol-5-yl-1H-indazol-5-amine (180 mg, 417.41 umol), zinc cyanide (147.04 mg, 1.25 mmol), Zn (81.88 mg, 1.25 mmol), 1,1-bis(diphenylphosphino)ferrocene (46.28 mg, 83.48 umol) and Pd₂dba₃ (76.45 mg, 83.48 umol) in DMA (9 mL) was degassed and purged with N₂ (3×) at 20° C., and then the mixture was stirred at 100° C. for 2 hrs under an N₂ atmosphere in a microwave reactor. The solvent was evaporated and the residue was purified by preparative-HPLC using Method BK to afford the title compound (70 mg, 44%) as a pale yellow solid. ¹H NMR (400 MHz, MeOD) δ 8.38 (s, 1H), 7.69-7.77 (m, 2H), 7.60-7.66 (m, 2H), 7.50 (d, J=9 Hz, 1H), 7.42 (s, 1H), 7.25 (dd, J=9, 2 Hz, 1H), 5.64 (dd, J=12, 9 Hz, 1H), 3.54-3.74 (m, 2H). MS-ESI (m/z) calc'd for C₂₀H₁₄F₂N₅O [M+H]⁺: 378.1. Found 378.2.

### Step 2: 2,2-Difluoro-1-((3-(oxazol-5-yl)-1H-indazol-5-yl)amino)-2,3-dihydro-1H-indene-5-carbonitrile enantiomer 1 and 2

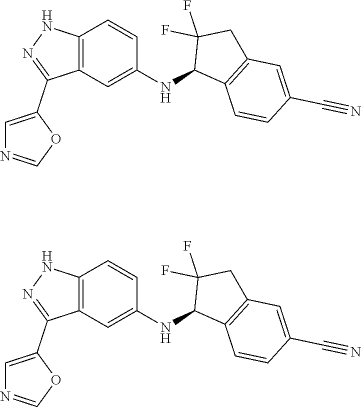 [SMILES: N#Cc1ccc2c(c1)CC(F)(F)[C@@H]2Nc1ccc2[nH]nc(-c3cnco3)c2c1 ; N#Cc1ccc2c(c1)CC(F)(F)[C@@H]2Nc1ccc2[nH]nc(-c3cnco3)c2c1]

2,2-Difluoro-1-((3-(oxazol-5-yl)-1H-indazol-5-yl)amino)-2,3-dihydro-1H-indene-5-carbonitrile was subjected to chiral separation using Method BL to afford 2,2-difluoro-1-((3-(oxazol-5-yl)-1H-indazol-5-yl)amino)-2,3-dihydro-1H-indene-5-carbonitrile, enantiomer 1 (22.44 mg, 32%). ¹H NMR (400 MHz, MeOD) (8.34 (s, 1H), 7.67-7.73 (m, 2H), 7.57-7.62 (m, 2H), 7.46 (d, J=9 Hz, 1H), 7.38 (d, J=2 Hz, 1H), 7.20 (dd, J=9, 2 Hz, 1H), 5.60 (dd, J=12, 9 Hz, 1H), 3.53-3.69 (m, 2H). MS-ESI (m/z) calc'd for C₂₀H₁₄F₂N₅O [M+H]⁺: 378.1. Found 378.1. A later eluting fraction was also isolated to afford 2,2-difluoro-1-((3-(oxazol-5-yl)-1H-indazol-5-yl)amino)-2,3-dihydro-1H-indene-5-carbonitrile, enantiomer 2 (17.71 mg, 25%). ¹H NMR (400 MHz, MeOD) (8.34 (s, 1H), 7.66-7.73 (m, 2H), 7.55-7.62 (m, 2H), 7.45 (d, J=9 Hz, 1H), 7.37 (s, 1H), 7.20 (dd, J=9, 2 Hz, 1H), 5.59 (br dd, J=12, 10 Hz, 1H), 3.52-3.68 (m, 2H). MS-ESI (m/z) calc'd for C₂₀H₁₄F₂N₅O [M+H]⁺: 378.1. Found 378.1.

### Example 43: 5-((3-Methyl-1H-indazol-5-yl)amino)-5,6,7,8-tetrahydronaphthalene-2-carbonitrile, enantiomer 1 and 2

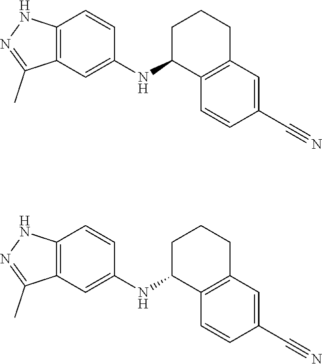 [SMILES: Cc1n[nH]c2ccc(N[C@H]3CCCc4cc(C#N)ccc43)cc12 ; Cc1n[nH]c2ccc(N[C@@H]3CCCc4cc(C#N)ccc43)cc12]

### Step 1: 3-Methyl-1H-indazol-5-amine

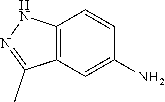

To a solution of 3-methyl-5-nitro-1H-indazole (200 mg, 1.13 mmol) in EtOH (3 mL) was added SnCl₂.2H₂O (1.02 g, 4.52 mmol) at 20° C. The mixture was stirred at 90° C. for 12 hrs. The reaction mixture was adjusted to pH=8 with a 2 N aqueous NaOH solution and filtered. The solid was washed with EtOH (50 ml) and filtered. The filtrate was concentrated to give a residue that was diluted with H₂O and extracted with EtOAc (3×). The combined organic phases were washed with brine, passed through a phase separator and evaporated to dryness to afford the title compound (60 mg, 36%) as a black solid. MS-ESI (m/z) calc'd for C₈H₁₀N₃ [M+H]⁺: 148.1. Found 148.1.

### Step 2: 5-((3-Methyl-1H-indazol-5-yl)amino)-5,6,7,8-tetrahydronaphthalene-2-carbonitrile

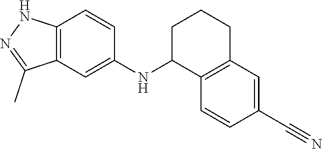 [SMILES: Cc1n[nH]c2ccc(NC3CCCc4cc(C#N)ccc43)cc12]

To a solution of 3-methyl-1H-indazol-5-amine (60 mg, 407.67 umol) and 1-oxotetralin-6-carbonitrile (34.90 mg, 203.84 umol) in toluene (5 mL) was added Ti(Oi-Pr)₄ (289.66 mg, 1.02 mmol) at 20° C. The mixture was stirred at 130° C. for 12 hrs. The mixture was concentrated and dissolved in MeOH (5 mL), then NaBH₄ (61.69 mg, 1.63 mmol) was added at 0° C. and the mixture was stirred at 20° C. for 4 hrs. The reaction mixture was evaporated to give a residue, the residue was diluted with H₂O and extracted with EtOAc (3×), the combined organic layers were dried over Na₂SO₄, then filtered and concentrated and purified by preparative-HPLC using Method CL to afford the title compound (9 mg, 11%) as a white solid. MS-ESI (m/z) calc'd for C₁₉H₁₉N₄ [M+H]⁺: 303.2. Found 303.2.

### Step 3: 5-((3-Methyl-1H-indazol-5-yl)amino)-5,6,7,8-tetrahydronaphthalene-2-carbonitrile, enantiomer 1 and 2

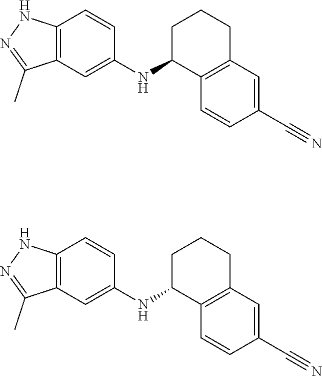 [SMILES: Cc1n[nH]c2ccc(N[C@H]3CCCc4cc(C#N)ccc43)cc12 ; Cc1n[nH]c2ccc(N[C@@H]3CCCc4cc(C#N)ccc43)cc12]

5-((3-Methyl-1H-indazol-5-yl)amino)-5,6,7,8-tetrahydronaphthalene-2-carbonitrile was subjected to chiral separation using Method BM to afford 5-((3-methyl-1H-indazol-5-yl)amino)-5,6,7,8-tetrahydronaphthalene-2-carbonitrile, enantiomer 1 (2.83 mg, 38%) as a white solid. ¹H NMR (400 MHz, MeOD) δ 7.59 (d, J=8.07 Hz, 1H), 7.49 (s, 1H), 7.45 (d, J=7.95 Hz, 1H), 7.27 (d, J=8.93 Hz, 1H), 6.96 (dd, J=8.93, 2.08 Hz, 1H), 6.84 (d, J=1.83 Hz, 1H), 4.64-4.73 (m, 1H), 2.78-2.95 (m, 2H), 2.46 (s, 3H), 1.85-2.09 (m, 4H). MS-ESI (m/z) calc'd for C₁₉H₁₉N₄ [M+H]⁺: 303.2. Found 303.1. A later eluting fraction was also isolated to afford 5-((3-methyl-1H-indazol-5-yl)amino)-5,6,7,8-tetrahydronaphthalene-2-carbonitrile, enantiomer 2 (3.39 mg, 31%) as a white solid. ¹H NMR (400 MHz, MeOD) δ 7.59 (d, J=7.95 Hz, 1H), 7.50 (s, 1H), 7.42-7.47 (m, 1H), 7.24-7.30 (m, 1H), 6.96 (dd, J=8.93, 2.08 Hz, 1H), 6.84 (s, 1H), 4.69 (br t, J=5.32 Hz, 1H), 2.78-2.96 (m, 2H), 2.46 (s, 3H), 1.85-2.09 (m, 4H). MS-ESI (m/z) calc'd for C₁₉H₁₉N₄ [M+H]⁺: 303.2. Found 303.1.

### Example 44: 5-((3-(Thiazol-5-yl)-1H-indazol-5-yl)amino)-5,6,7,8-tetrahydronaphthalene-2-carbonitrile, enantiomer 1

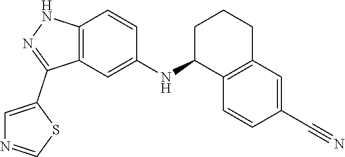 [SMILES: N#Cc1ccc2c(c1)CCC[C@@H]2Nc1ccc2[nH]nc(-c3cncs3)c2c1]

A mixture of 5-((3-iodo-1H-indazol-5-yl)amino)-5,6,7,8-tetrahydronaphthalene-2-carbonitrile, enantiomer 1 (20 mg, 48.28 umol), 5-(tributylstannyl)thiazole (23.48 mg, 62.77 umol) and PdCl₂(PPh₃)₂ (3.39 mg, 4.83 umol) in DMF (1 mL) was degassed and purged with N₂ (3×) at 20° C., and then the mixture was stirred at 120° C. for 12 hrs under an N₂ atmosphere. The reaction mixture was evaporated to give a residue that was purified by preparative-HPLC using Method BN to afford the title compound (3.02 mg, 12%) as a yellow solid. ¹H NMR (400 MHz, DMSO-d₆) δ 13.01 (br s, 1H), 9.04 (s, 1H), 8.43 (s, 1H), 7.64 (s, 1H), 7.59-7.52 (m, 2H), 7.37 (d, J=9.0 Hz, 1H), 7.14 (s, 1H), 7.04-6.98 (m, 1H), 4.90 (br s, 1H), 2.89-2.77 (m, 2H), 2.04-1.79 (m, 4H). MS-ESI (m/z) calc'd for C₂₁H₁₈N₅S [M+H]⁺: 372.1. Found 372.0.

### Example 45: 5-((3-Cyclohexyl-1H-indazol-5-yl)amino)-5,6,7,8-tetrahydronaphthalene-2-carbonitrile, enantiomer 1

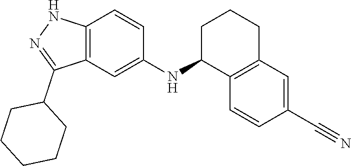 [SMILES: N#Cc1ccc2c(c1)CCC[C@@H]2Nc1ccc2[nH]nc(C3CCCCC3)c2c1]

A mixture of 5-((3-(cyclohex-1-en-1-yl)-1H-indazol-5-yl)amino)-5,6,7,8-tetrahydronaphthalene-2-carbonitrile, enantiomer 1 (18 mg, 48.85 umol) and 10% Pd/C (40 mg, 48.85 umol) in MeOH (1 mL) was degassed and purged with H₂ (3×), and then the mixture was stirred at 20° C. for 4 hrs under an H₂ atmosphere (15 psi). The mixture was filtered and the filtrate was concentrated to give a residue. The residue was purified by preparative-HPLC using Method BO to afford the title compound (4.06 mg, 16%) as a white solid. ¹H NMR (400 MHz, DMSO-d₆) δ 7.67 (s, 1H), 7.63-7.58 (m, 1H), 7.58-7.52 (m, 1H), 7.33 (br d, J=7.1 Hz, 1H), 7.22-6.79 (m, 2H), 4.78 (br s, 1H), 2.97-2.73 (m, 3H), 1.96-1.67 (m, 9H), 1.66-1.51 (m, 2H), 1.40 (q, J=12.5 Hz, 2H), 1.32-1.21 (m, 1H). MS-ESI (m/z) calc'd for C₂₄H₂₇N₄ [M+H]⁺: 371.2. Found 371.1.

### Example 46: 5-((3-(1-Methyl-2-oxo-1,2-dihydropyridin-3-yl)-1H-indazol-5-yl)amino)-5,6,7,8-tetrahydronaphthalene-2-carbonitrile, enantiomer 1

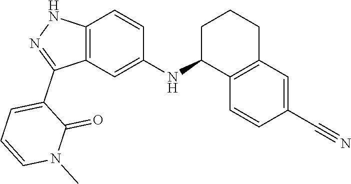 [SMILES: Cn1cccc(-c2n[nH]c3ccc(N[C@H]4CCCc5cc(C#N)ccc54)cc23)c1=O]

### Step 1: (1-Methyl-2-oxo-1,2-dihydropyridin-3-yl)boronic acid

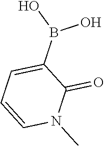

A mixture of 3-bromo-1-methyl-pyridin-2-one (70 mg, 372.30 umol), KOAc (109.61 mg, 1.12 mmol), and Pd(dppf)Cl₂DCM (15.20 mg, 18.61 umol) in 1,4-dioxane (2 mL) was degassed and purged with N₂ (3×) at 20° C. The mixture was then stirred at 100° C. for 2 hrs under an N₂ atmosphere. The reaction mixture was evaporated to dryness to afford the title compound (50 mg, 60%) as a black solid. MS-ESI (m/z) calc'd for C₆H₉BNO₃ [M+H]⁺: 154.1. Found 154.1.

### Step 2: 5-((3-(1-Methyl-2-oxo-1,2-dihydropyridin-3-yl)-H-indazol-5-yl)amino)-5,6,7,8-tetrahydronaphthalene-2-carbonitrile, enantiomer 1

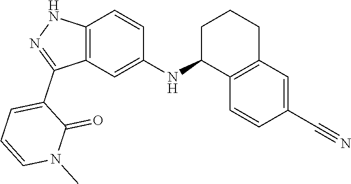 [SMILES: Cn1cccc(-c2n[nH]c3ccc(N[C@H]4CCCc5cc(C#N)ccc54)cc23)c1=O]

A mixture of 5-((3-iodo-1H-indazol-5-yl)amino)-5,6,7,8-tetrahydronaphthalene-2-carbonitrile (20 mg, 48.28 umol), enantiomer 1, (1-methyl-2-oxo-1,2-dihydropyridin-3-yl)boronic acid (44.31 mg, 289.69 umol), Pd(amphos)Cl₂ (3.42 mg, 4.83 umol) and AcOK (14.22 mg, 144.84 umol) in EtOH (2 mL) and H₂O (0.5 mL) was degassed and purged with N₂ (3×) at 20° C., and then the mixture was stirred at 100° C. for 12 hrs under an N₂ atmosphere. The reaction mixture was evaporated to give a residue that was purified by preparative-HPLC using Method BP to give material of insufficient purity. The material was then re-purified by preparative-HPLC using Method BQ to afford the title compound (4.97 mg, 24%) as a yellow solid. ¹H NMR (400 MHz, DMSO-d₆) δ 12.72 (br s, 1H), 7.75 (dd, J=1.9, 6.7 Hz, 1H), 7.61 (s, 1H), 7.57-7.51 (m, 2H), 7.39-7.10 (m, 2H), 6.96 (s, 1H), 6.94-6.89 (m, 1H), 6.31 (br s, 1H), 5.67 (br s, 1H), 4.58 (br s, 1H), 3.62-3.42 (m, 3H), 2.88-2.72 (m, 2H), 1.96-1.74 (m, 4H). MS-ESI (m/z) calc'd for C₂₄H₂₂N₅O [M+H]⁺: 396.2. Found 396.1.

### Example 47: 7-((3-(Oxazol-5-yl)-1H-indazol-5-yl)amino)-4,5,6,7-tetrahydrobenzo[d]thiazole-2-carbonitrile

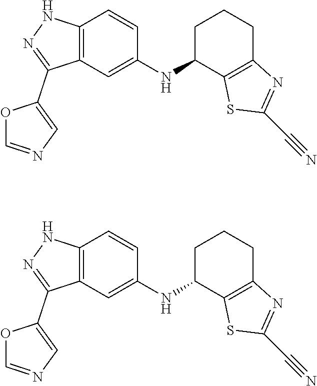 [SMILES: N#Cc1nc2c(s1)[C@@H](Nc1ccc3[nH]nc(-c4cnco4)c3c1)CCC2 ; N#Cc1nc2c(s1)[C@H](Nc1ccc3[nH]nc(-c4cnco4)c3c1)CCC2]

### Step 1: 2-Bromo-N-(3-(oxazol-5-yl)-1H-indazol-5-yl)-4,5,6,7-tetrahydrobenzo[d]thiazol-7-amine

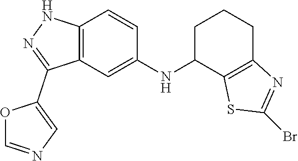 [SMILES: Brc1nc2c(s1)C(Nc1ccc3[nH]nc(-c4cnco4)c3c1)CCC2]

To a solution of 2-bromo-5,6-dihydro-4H-1,3-benzothiazol-7-one (1.02 g, 4.40 mmol) and 3-oxazol-5-yl-1H-indazol-5-amine (880 mg, 4.40 mmol) in toluene (30 mL) was added Ti(i-PrO)₄ (6.25 g, 21.98 mmol) at 20° C. The mixture was stirred at 120° C. for 12 hrs and then concentrated to give a residue. The residue was diluted with MeOH (30 mL) and NaBH₄ (1.33 g, 35.17 mmol) was added at 0° C. and the mixture was then stirred at 20° C. for 12 hrs. The reaction mixture was concentrated to remove MeOH. The residue was diluted with EtOAc and H₂O, filtered and the filtrate was passed through a phase separator and evaporated to give a residue. The residue was purified by flash silica gel column chromatography (ISCO; 12 g SepaFlash column) using a 0-50% EtOAc/petroleum ether gradient eluent to afford the title compound (850 mg, 20%) as a yellow solid. MS-ESI (m/z) calc'd for C₁₇H₁₅BrN₅OS [M+H]⁺: 418.0/416.0. Found 418.0/416.0.

### Step 2: 7-((3-(Oxazol-5-yl)-1H-indazol-5-yl)amino)-4,5,6,7-tetrahydrobenzo[d]thiazole-2-carbonitrile

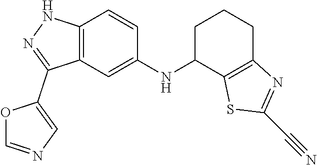 [SMILES: N#Cc1nc2c(s1)C(Nc1ccc3[nH]nc(-c4cnco4)c3c1)CCC2]

A mixture of 2-bromo-N-(3-(oxazol-5-yl)-1H-indazol-5-yl)-4,5,6,7-tetrahydrobenzo[d]thiazol-7-amine (400 mg, 960.86 umol), Zn(CN)₂ (225.67 mg, 1.92 mmol), Zn (43.98 mg, 672.60 umol), Pd₂dba₃ (87.99 mg, 96.09 umol) and 1,1-bis(diphenylphosphino)ferrocene (106.54 mg, 192.17 umol) in DMA (4 mL) was degassed and purged with N₂ (3×) at 20° C. The mixture was then stirred at 80° C. for 12 hrs under an N₂ atmosphere followed by stirring at 100° C. for an additional 12 hrs. The mixture was filtered and the filtrate was concentrated under vacuum. The residue was diluted with MeOH and filtered. The filtrate was purified by preparative-HPLC using Method BR and further purified by preparative-HPLC using Method BS to afford the title compound (20.38 mg, 4%) as a yellow solid. ¹H NMR (400 MHz, DMSO-d₆) δ 13.16 (br s, 1H), 8.46 (s, 1H), 7.73 (s, 1H), 7.42 (d, J=8.8 Hz, 1H), 7.32-7.18 (m, 2H), 6.99 (dd, J=2.0, 9.0 Hz, 1H), 5.17 (br t, J=6.4 Hz, 1H), 2.90-2.80 (m, 2H), 2.27-2.17 (m, 1H), 2.15-2.05 (m, 1H), 1.96-1.84 (m, 1H), 1.82-1.71 (m, 1H). MS-ESI (m/z) calc'd for C₁₈H₁₅N₆OS [M+H]⁺: 363.1. Found 363.0.

### Step 6: 7-((3-(Oxazol-5-yl)-1H-indazol-5-yl)amino)-4,5,6,7-tetrahydrobenzo[d]thiazole-2-carbonitrile, enantiomer 1 and 2

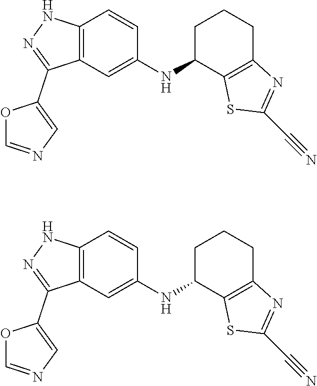 [SMILES: N#Cc1nc2c(s1)[C@@H](Nc1ccc3[nH]nc(-c4cnco4)c3c1)CCC2 ; N#Cc1nc2c(s1)[C@H](Nc1ccc3[nH]nc(-c4cnco4)c3c1)CCC2]

7-((3-(Oxazol-5-yl)-1H-indazol-5-yl)amino)-4,5,6,7-tetrahydrobenzo[d]thiazole-2-carbonitrile was subjected to chiral separation using Method BT to afford 7-((3-(oxazol-5-yl)-1H-indazol-5-yl)amino)-4,5,6,7-tetrahydrobenzo[d]thiazole-2-carbonitrile, enantiomer 1 (2.35 mg, 25%) as a yellow solid. ¹H NMR (400 MHz, DMSO-d₆) δ 13.21 (br s, 1H), 8.49 (s, 1H), 7.76 (s, 1H), 7.44 (d, J=9.0 Hz, 1H), 7.21 (s, 1H), 7.00 (dd, J=1.8, 9.0 Hz, 1H), 6.13 (d, J=10.3 Hz, 1H), 5.26-5.13 (m, 1H), 2.95-2.79 (m, 2H), 2.28-2.06 (m, 2H), 1.97-1.72 (m, 2H). MS-ESI (m/z) calc'd for C₁₈H₁₅N₆OS [M+H]⁺: 363.1. Found 363.0. A later eluting fraction was also isolated to afford 7-((3-(oxazol-5-yl)-1H-indazol-5-yl)amino)-4,5,6,7-tetrahydrobenzo[d]thiazole-2-carbonitrile, enantiomer 2 (2.64 mg, 28%) as a yellow solid. ¹H NMR (400 MHz, DMSO-d₆) δ 13.21 (br s, 1H), 8.49 (s, 1H), 7.76 (s, 1H), 7.44 (d, J=8.9 Hz, 1H), 7.21 (s, 1H), 7.05-6.97 (m, 1H), 6.13 (d, J=10.1 Hz, 1H), 5.19 (br s, 1H), 2.95-2.79 (m, 2H), 2.28-2.04 (m, 2H), 1.98-1.72 (m, 2H). MS-ESI (m/z) calc'd for C₁₈H₁₅N₆OS [M+H]⁺: 363.1. Found 363.0.

### Example 48: 6-((3-(Oxazol-5-yl)-1H-indazol-5-yl)amino)-4b,5,5a,6-tetrahydrocyclopropa[3,4]cyclopenta[1,2-b]pyridine-3-carbonitrile

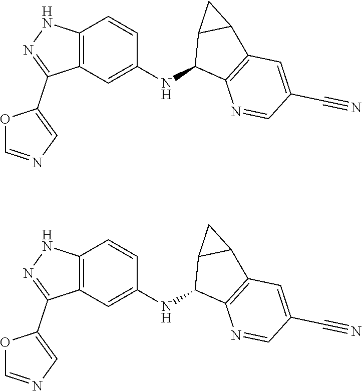 [SMILES: N#Cc1cnc2c(c1)C1CC1[C@@H]2Nc1ccc2[nH]nc(-c3cnco3)c2c1 ; N#Cc1cnc2c(c1)C1CC1[C@H]2Nc1ccc2[nH]nc(-c3cnco3)c2c1]

### Step 1: 3-Bromo-6,7-dihydro-5H-cyclopenta[b]pyridine

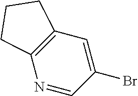

To a mixture of 6,7-dihydro-5H-cyclopenta[b]pyridine (20 g, 167.84 mmol) and AlCl₃ (55.95 g, 419.59 mmol) was added Br₂ (31.11 g, 194.69 mmol) dropwise at 100° C. over 5 minutes. The mixture was then stirred at 100° C. for another 55 minutes. The reaction mixture was poured into ice water slowly and basified with saturated aqueous Na₂CO₃ to pH=7 and the mixture was filtered. The filtrate was extracted with EtOAc (3×) and the combined organic layers were dried over Na₂SO₄ and evaporated to give a residue that was purified by flash silica gel chromatography (ISCO; 220 g SepaFlash column) using a 0-10% EtOAc/petroleum ether gradient eluent to afford the title compound (25.8 g, 75%) as a white solid. ¹H NMR (400 MHz, CDCl₃) δ 8.23-8.44 (m, 1H), 7.47-7.67 (m, 1H), 2.77-3.03 (m, 4H), 1.98-2.21 (m, 2H). MS-ESI (m/z) calc'd for C₈H₉BrN [M+H]⁺: 198.0/222.0. Found 198.1/200.1.

### Step 2: 3-Bromo-6,7-dihydro-5H-cyclopenta[b]pyridine 1-oxide

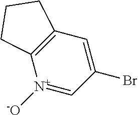

To a solution of 3-bromo-6,7-dihydro-5H-cyclopenta[b]pyridine (10 g, 50.49 mmol) in DCE (300 mL) was added MCPBA (21.78 g, 100.98 mmol) at 20° C. The mixture was then stirred at 70° C. for 12 hrs, quenched with saturated aqueous Na₂SO₃, and extracted with DCM (4×). The combined organic layers were dried over Na₂SO₄ and evaporated to afford the title compound (10 g, 92%) as a brown gum. MS-ESI (m/z) calc'd for C₈H₉BrNO [M+H]⁺: 214.0/216.0. Found 213.9/216.0.

### Step 3: 3-Bromo-6,7-dihydro-5H-cyclopenta[b]pyridin-7-yl acetate

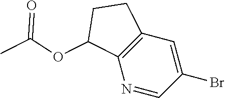

A solution of 3-bromo-6,7-dihydro-5H-cyclopenta[b]pyridine 1-oxide (10 g, 46.72 mmol) in Ac₂O (33.38 g, 327.01 mmol) was stirred at 60° C. for 12 hrs. The reaction mixture was evaporated to give a residue that was purified by flash silica gel chromatography (ISCO; 40 g SepaFlash column) using a 0-5% EtOAc/petroleum ether gradient eluent to afford the title compound (8.8 g, 74%) as a red gum. MS-ESI (m/z) calc'd for C₁₀H₁₁BrNO₂ [M+H]⁺: 256.0/258.0. Found 256.0/258.0.

### Step 4: 3-Bromo-7H-cyclopenta[b]pyridine and 3-Bromo-5H-cyclopenta[b]pyridine

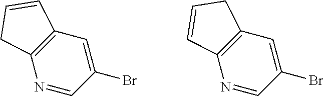 [SMILES: Brc1cnc2c(c1)C=CC2 ; Brc1cnc2c(c1)CC=C2]

A solution of (3-bromo-6,7-dihydro-5H-cyclopenta[b]pyridin-7-yl) acetate (8.8 g, 34.36 mmol) in H₂SO₄ (12.34 mL, 226.79 mmol) was stirred at 120° C. for 2 hrs. The reaction mixture was poured into ice water and basified with solid Na₂CO₃ to pH=8. The mixture was then extracted with EtOAc (3×) and the combined organic layers were dried over Na₂SO₄ and evaporated to give a residue. The residue was purified by flash silica gel chromatography (ISCO; 40 g SepaFlash column) using a 0-8% EtOAc/petroleum ether gradient eluent to afford a 2:1 mixture of the title compounds (4.38 g, 65%) as a pink solid. 3-Bromo-7H-cyclopenta[b]pyridine: ¹H NMR (400 MHz, DMSO-d₆) δ 8.52 (d, J=2 Hz, 1H), 8.07-8.11 (m, 1H), 7.05 (dt, J=6, 2 Hz, 1H), 6.96-7.00 (m, 1H), 3.50 (s, 2H). 3-Bromo-5H-cyclopenta[b]pyridine: ¹H NMR (400 MHz, DMSO-d₆) δ 8.39 (d, J=2 Hz, 1H), 7.99 (d, J=2 Hz, 1H), 6.95 (br s, 1H), 6.76-6.79 (m, 1H), 3.47 (s, 2H). MS-ESI (m/z) calc'd for C₈H₇BrN [M+H]⁺: 196.0/198.0. Found 196.0/198.0.

### Step 5: 3-Bromo-4b,5,5a,6-tetrahydrocyclopropa[3,4]cyclopenta[1,2-b]pyridine and 3-Bromo-5,5a,6,6a-tetrahydrocyclopropa[4,5]cyclopenta[1,2-b]pyridine

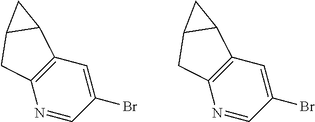 [SMILES: Brc1cnc2c(c1)C1CC1C2 ; Brc1cnc2c(c1)C1CC1C2]

To a solution of KOH aqueous (320 mL, 50%) and diethyl ether (350 mL) was added 1-methyl-1-nitrosourea (10.52 g, 102.02 mmol) slowly at 0° C. and the mixture was stirred at 0° C. for 10 minutes. The ether layer was separated and used in the subsequent step. To a solution of 3-bromo-7H-cyclopenta[b]pyridine and 3-bromo-5H-cyclopenta[b]pyridine (2 g, 10.20 mmol) in diethyl ether (50 mL) was added palladium acetate (229.04 mg, 1.02 mmol) and the mixture was purged and degassed with N₂ (3×). The prepared ether layer was then added to the mixture at 0° C. Stirring was then continued at 20° C. for 12 hrs under an N₂ atmosphere. The reaction mixture was poured into an 20% AcOH aqueous solution; then the mixture was basified with saturated aqueous Na₂CO₃ to pH=7 and extracted with EtOAc (4×). The combined organic layers were dried over Na₂SO₄, filtered, and the solvent was evaporated to give a residue. The residue was purified by flash silica gel chromatography (ISCO; 20 g SepaFlash column) using a 0-8% EtOAc/petroleum ether gradient eluent to afford a 1:1 mixture of the title compounds (780 mg, 36%) as a red gum. 3-Bromo-4b,5,5a,6-tetrahydrocyclopropa[3,4]cyclopenta[1,2-b]pyridine: MS-ESI (m/z) calc'd for C₉H₉BrN [M+H]⁺: 210.0/212.0. Found 210.2/212.2. 3-Bromo-5,5a,6,6a-tetrahydrocyclopropa[4,5]cyclopenta[1,2-b]pyridine: MS-ESI (m/z) calc'd for C₉H₉BrN [M+H]⁺: 210.0/212.0. Found 210.2/212.2.

### Step 6: 3-Bromo-4b,5,5a,6-tetrahydrocyclopropa[3,4]cyclopenta[1,2-b]pyridine 1-oxide and 3-Bromo-5,5a,6,6a-tetrahydrocyclopropa[4,5]cyclopenta[1,2-b]pyridine 1-oxide

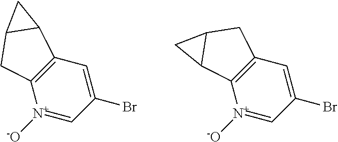

To a solution of 3-bromo-4b,5,5a,6-tetrahydrocyclopropa[3,4]cyclopenta[1,2-b]pyridine and 3-bromo-5,5a,6,6a-tetrahydrocyclopropa[4,5]cyclopenta[1,2-b]pyridine (780 mg, 3.71 mmol) in DCM (25 mL) was added MCPBA (1.20 g, 5.57 mmol). The mixture was stirred at 20° C. for 12 hrs and then quenched with saturated aqueous Na₂CO₃ and stirred at 25° C. for 0.5 hr. The mixture was extracted with DCM (3×); the combined organic layers were dried over Na₂SO₄, filtered and concentrated to afford a 1:1 mixture of the title compounds (735 mg, 87%) as a brown gum. 3-Bromo-4b,5,5a,6-tetrahydrocyclopropa[3,4]cyclopenta[1,2-b]pyridine 1-oxide: MS-ESI (m/z) calc'd for C₉H₉BrNO [M+H]⁺: 226.0/228.0. Found 226.1/228.1. 3-Bromo-5,5a,6,6a-tetrahydrocyclopropa[4,5]cyclopenta[1,2-b]pyridine 1-oxide: MS-ESI (m/z) calc'd for C₉H₉BrNO [M+H]⁺: 226.0/228.0. Found 226.1/228.1.

### Step 7: 3-Bromo-4b,5,5a,6-tetrahydrocyclopropa[3,4]cyclopenta[1,2-b]pyridin-6-ol

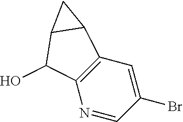 [SMILES: OC1c2ncc(Br)cc2C2CC12]

A mixture of 3-bromo-4b,5,5a,6-tetrahydrocyclopropa[3,4]cyclopenta[1,2-b]pyridine 1-oxide and 3-bromo-5,5a,6,6a-tetrahydrocyclopropa[4,5]cyclopenta[1,2-b]pyridine 1-oxide (685 mg, 3.03 mmol) and TFAA (5 mL, 35.95 mmol) in DCM (5 mL) was stirred at 40° C. for 12 hrs. The reaction mixture was evaporated to give a residue that was diluted with 2M NaOH and extracted with EtOAc (3×). The combined organic layers were dried over Na₂SO₄ and the solvent was evaporated. The material was purified by flash silica gel chromatography (ISCO; 12 g SepaFlash column) using a 0-35% EtOAc/petroleum ether gradient eluent to afford the title compound (127 mg, 18%) as a yellow gum. MS-ESI (m/z) calc'd for C₉H₉BrNO [M+H]⁺: 226.0/228.0. Found 226.2/228.1.

### Step 8: 3-Bromo-5,5a-dihydrocyclopropa[3,4]cyclopenta[1,2-b]pyridin-6(4bH)-one

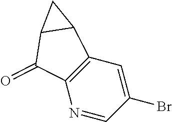 [SMILES: O=C1c2ncc(Br)cc2C2CC12]

To a solution of 3-bromo-4b,5,5a,6-tetrahydrocyclopropa[3,4]cyclopenta[1,2-b]pyridin-6-ol (127 mg, 561.77 umol) in DCM (5 mL) was added Dess-Martin periodinane (285.93 mg, 674.13 umol) and the mixture was stirred at 20° C. for 12 hrs. The solvent was evaporated to give a residue that was purified by preparative-TLC (petroleum ether/EtOAc=1/1, Rf=0.43) to afford the title compound (110 mg, 87%) as a yellow oil. ¹H NMR (400 MHz, CDCl₃) δ 8.63 (d, J=2 Hz, 1H), 7.92 (d, J=2 Hz, 1H), 2.86 (dt, J=7, 4 Hz, 1H), 2.57 (dt, J=9, 4 Hz, 1H), 1.66 (ddd, J=9, 7, 5 Hz, 1H), 1.37 (q, J=4 Hz, 1H). MS-ESI (m/z) calc'd for C₉H₇BrNO [M+H]⁺: 224.0/226.0. Found 224.1/226.1.

### Step 9: 3-Bromo-N-(3-(oxazol-5-yl)-1H-indazol-5-yl)-4b,5,5a,6-tetrahydrocyclopropa[3,4]cyclopenta[1,2-b]pyridin-6-amine

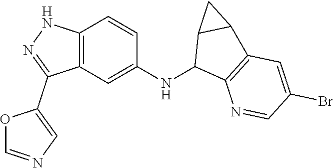 [SMILES: Brc1cnc2c(c1)C1CC1C2Nc1ccc2[nH]nc(-c3cnco3)c2c1]

To a solution of 3-oxazol-5-yl-1H-indazol-5-amine (85 mg, 424.58 umol) and 3-bromo-5,5a-dihydrocyclopropa[3,4]cyclopenta[1,2-b]pyridin-6(4bH)-one (95.13 mg, 424.58 umol) in toluene (5 mL) was added Ti(Oi-Pr)₄ (603.35 mg, 2.12 mmol) and the mixture was stirred at 100° C. for 12 hrs. After cooling to 20° C., the mixture was evaporated to give a residue that was dissolved in MeOH (5 mL). NaBH₄ (128.50 mg, 3.40 mmol) was then added to the mixture at 0° C. and the mixture was stirred at 20° C. for an additional 4 hrs. The solvent was evaporated to give a residue that was purified by preparative-TLC (100% EtOAc) to afford the title compound (73 mg, 42%) as a gray solid. MS-ESI (m/z) calc'd for C₁₉H₁₅BrN₅O [M+H]⁺: 408.0/410.0. Found 408.0/410.0.

### Step 10: 6-((3-(Oxazol-5-yl)-1H-indazol-5-yl)amino)-4b,5,5a,6-tetrahydrocyclopropa[3,4]cyclopenta[1,2-b]pyridine-3-carbonitrile

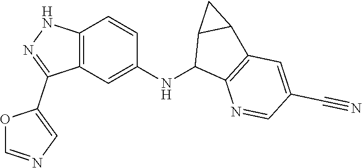 [SMILES: N#Cc1cnc2c(c1)C1CC1C2Nc1ccc2[nH]nc(-c3cnco3)c2c1]

A mixture of 3-bromo-N-(3-(oxazol-5-yl)-1H-indazol-5-yl)-4b,5,5a,6-tetrahydrocyclopropa[3,4]cyclopenta[1,2-b]pyridin-6-amine (30 mg, 73.48 umol), zinc cyanide (25.89 mg, 220.45 umol), Zn (4.81 mg, 73.48 umol), 1,1′-bis(diphenylphosphino)ferrocene (8.15 mg, 14.70 umol) and Pd₂dba₃ (13.46 mg, 14.70 umol) in DMA (2 mL) was degassed and purged with N₂ (3×) at 20° C. and then stirred at 120° C. for 2 hrs under an N₂ atmosphere in a microwave reactor. The reaction mixture was filtered and the filtrate was evaporated to give a residue that was purified by preparative-HPLC using Method BW to afford the title compound (9 mg, 25%) as a yellow solid. MS-ESI (m/z) calc'd for C₁₉H₁₅BrN₅O [M+H]⁺: 355.1. Found 355.1.

### Step 11: 6-((3-(Oxazol-5-yl)-1H-indazol-5-yl)amino)-4b,5,5a,6-tetrahydrocyclopropa[3,4]cyclopenta[1,2-b]pyridine-3-carbonitrile, enantiomer 1 and 2

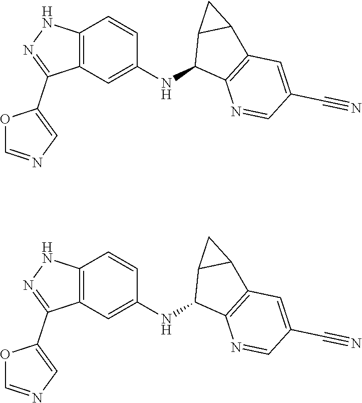 [SMILES: N#Cc1cnc2c(c1)C1CC1[C@@H]2Nc1ccc2[nH]nc(-c3cnco3)c2c1 ; N#Cc1cnc2c(c1)C1CC1[C@H]2Nc1ccc2[nH]nc(-c3cnco3)c2c1]

6-((3-(Oxazol-5-yl)-1H-indazol-5-yl)amino)-4b,5,5a,6-tetrahydrocyclopropa[3,4]cyclopenta[1,2-b]pyridine-3-carbonitrile was subjected to chiral separation using Method BX to afford 6-((3-(oxazol-5-yl)-1H-indazol-5-yl)amino)-4b,5,5a,6-tetrahydrocyclopropa[3,4]cyclopenta[1,2-b]pyridine-3-carbonitrile, enantiomer 1 (3.46 mg, 52%) as a yellow solid. ¹H NMR (400 MHz, DMSO-d₆) δ 13.12 (br s, 1H), 8.78 (d, J=2 Hz, 1H), 8.47 (s, 1H), 8.22 (d, J=2 Hz, 1H), 7.73 (s, 1H), 7.40 (d, J=9 Hz, 1H), 7.22 (s, 1H), 7.08 (dd, J=9, 2 Hz, 1H), 5.92 (d, J=8 Hz, 1H), 5.51 (t, J=7 Hz, 1H), 2.55-2.61 (m, 1H), 2.42-2.46 (m, 1H), 1.06 (td, J=8, 5 Hz, 1H), 0.58 (q, J=4 Hz, 1H). MS-ESI (m/z) calc'd for C₁₉H₁₅BrN₅O [M+H]⁺: 355.1. Found 355.0. A later eluting fraction was also isolated to afford 6-((3-(oxazol-5-yl)-1H-indazol-5-yl)amino)-4b,5,5a,6-tetrahydrocyclopropa[3,4]cyclopenta[1,2-b]pyridine-3-carbonitrile, enantiomer 2 (2.83 mg, 43%) as a yellow solid. ¹H NMR (400 MHz, DMSO-d₆) δ 13.12 (s, 1H), 8.78 (d, J=2 Hz, 1H), 8.47 (s, 1H), 8.22 (d, J=2 Hz, 1H), 7.73 (s, 1H), 7.40 (d, J=9 Hz, 1H), 7.22 (s, 1H), 7.08 (dd, J=9, 2 Hz, 1H), 5.92 (d, J=8 Hz, 1H), 5.50 (t, J=7 Hz, 1H), 2.55-2.60 (m, 1H), 2.42-2.46 (m, 1H), 1.01-1.08 (m, 1H), 0.58 (q, J=4 Hz, 1H). MS-ESI (m/z) calc'd for C₁₉H₁₅BrN₅O [M+H]⁺: 355.1. Found 355.0.

### Example 49: 8-((3-(1-(Difluoromethyl)-1H-pyrazol-4-yl)-1H-indazol-5-yl)amino)-5,6,7,8-tetrahydroquinoline-3-carbonitrile, enantiomer 1 and 2

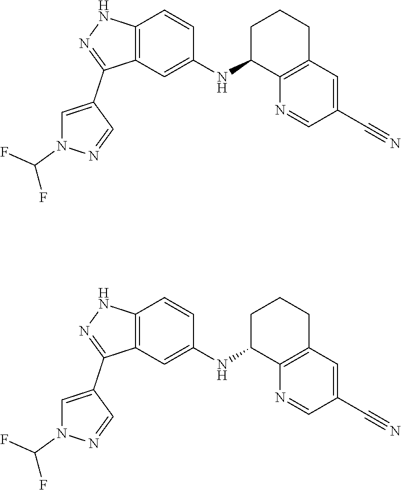 [SMILES: N#Cc1cnc2c(c1)CCC[C@@H]2Nc1ccc2[nH]nc(-c3cnn(C(F)F)c3)c2c1 ; N#Cc1cnc2c(c1)CCC[C@H]2Nc1ccc2[nH]nc(-c3cnn(C(F)F)c3)c2c1]

### Step 1: 3-Bromo-5,6,7,8-tetrahydroquinoline 1-oxide

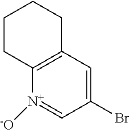

To a solution of 3-bromo-5,6,7,8-tetrahydroquinoline (1 g, 4.72 mmol) in DCM (15 mL) was added MCPBA (2.03 g, 9.43 mmol) and the mixture was stirred at 40° C. for 2 hrs. The reaction mixture was then cooled to 0° C. and quenched by addition of 10% aqueous Na₂SO₃ (10 mL) and filtered. The filtrate was extracted with DCM (3×). The combined organic layers were passed through a phase separator and concentrated to afford the title compound (1 g, 92%) as a yellow solid. MS-ESI (m/z) calc'd for C₉H₁₁BrNO [M+H]⁺: 228.0/230.0. Found 228.1/230.1.

### Step 2: 3-Bromo-5,6,7,8-tetrahydroquinolin-8-yl acetate

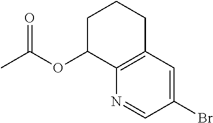

A mixture of 3-bromo-5,6,7,8-tetrahydroquinoline 1-oxide (1 g, 4.38 mmol) in acetic anhydride (5.13 g, 50.24 mmol) was stirred at 50° C. for 12 hrs. The reaction mixture was concentrated and the residue was adjusted to pH=8 with saturated aqueous NaHCO₃ (10 mL) and extracted with EtOAc (3×). The combined organic layers were passed through a phase separator and concentrated to afford the title compound (1 g, 84%) as a yellow oil. MS-ESI (m/z) calc'd for C₁₁H₁₃BrNO₂ [M+H]⁺: 270.0/272.0 Found 270.0/272.0.

### Step 3: 3-Bromo-5,6,7,8-tetrahydroquinolin-8-ol

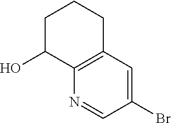 [SMILES: OC1CCCc2cc(Br)cnc21]

To a solution of 3-bromo-5,6,7,8-tetrahydroquinolin-8-yl acetate (1 g, 3.70 mmol) in MeOH (10 mL) was added K₂CO₃ (2.05 g, 14.81 mmol). The mixture was stirred at 20° C. for 12 hrs. The mixture was filtered and the filtrate was evaporated to give a residue that was purified by flash silica gel chromatography (ISCO; 12 g SepaFlash column) using a 0-23% EtOAc/petroleum ether gradient eluent to afford the title compound (500 mg, 59%) as a yellow oil. ¹H NMR (400 MHz, DMSO-d₆) δ 8.49 (d, J=2.21 Hz, 1H), 7.77 (s, 1H), 5.21 (d, J=4.41 Hz, 1H), 4.50-4.55 (m, 1H), 2.63-2.83 (m, 2H), 1.80-1.93 (m, 3H), 1.61-1.71 (m, 1H). MS-ESI (m/z) calc'd for C₉H₁₁BrNO [M+H]⁺: 228.0/230.0. Found 228.0/230.0.

### Step 4: 3-Bromo-6,7-dihydroquinolin-8(5H)-one

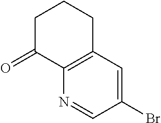 [SMILES: O=C1CCCc2cc(Br)cnc21]

To a solution of 3-bromo-5,6,7,8-tetrahydroquinolin-8-ol (500 mg, 2.19 mmol) in DCM (50 mL) was added Dess-Martin periodinane (3.72 g, 8.77 mmol). The mixture was stirred at 20° C. for 12 hrs. The mixture was then filtered and the filtrate was evaporated to give a residue that was purified by flash silica gel chromatography (ISCO; 12 g SepaFlash column) using a 0-36% EtOAc/petroleum ether gradient eluent to afford the title compound (400 mg, 81%) as a yellow solid. ¹H NMR (400 MHz, DMSO-d₆) δ 8.71 (s, 1H), 8.18 (s, 1H), 3.00 (t, J=5.95 Hz, 1H), 2.66-2.71 (m, 1H), 2.51-2.52 (m, 1H), 2.01-2.11 (s, 1H), 1.66 (s, 2H). MS-ESI (m/z) calc'd for C₉H₉BrNO [M+H]⁺: 226.0/228.0. Found 225.9/228.0.

### Step 5: 3-Bromo-N-(3-(1-(difluoromethyl)-1H-pyrazol-4-yl)-1H-indazol-5-yl)-5,6,7,8-tetrahydroquinolin-8-amine

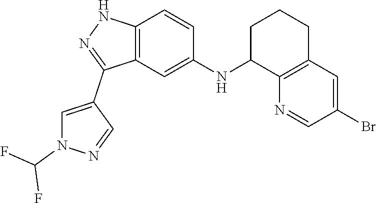 [SMILES: FC(F)n1cc(-c2n[nH]c3ccc(NC4CCCc5cc(Br)cnc54)cc23)cn1]

To a solution of 3-[1-(difluoromethyl)pyrazol-4-yl]-1H-indazol-5-amine (200 mg, 802.51 umol) and 3-bromo-6,7-dihydroquinolin-8(5H)-one (181.42 mg, 802.51 umol) in MeOH (15 mL) was added acetic acid (96.38 mg, 1.61 mmol) to adjust pH=5. The mixture was then stirred at 20° C. for 1 hr. NaBH₃CN (151.29 mg, 2.41 mmol) was added and the mixture was stirred at 20° C. for 12 hrs. The solution was evaporated and the residue was purified by flash silica gel chromatography (ISCO; 12 g SepaFlash column) using a 0-70% EtOAc/petroleum ether gradient eluent to afford the title compound (300 mg, 81%) as a yellow oil. MS-ESI (m/z) calc'd for C₂₀H₁₈BrF₂N₆ [M+H]⁺: 459.1/461.1. Found 459.0/461.0.

### Step 6: 8-((3-(1-(Difluoromethyl)-1H-pyrazol-4-yl)-1H-indazol-5-yl)amino)-5,6,7,8-tetrahydroquinoline-3-carbonitrile

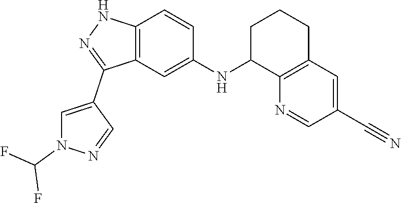 [SMILES: N#Cc1cnc2c(c1)CCCC2Nc1ccc2[nH]nc(-c3cnn(C(F)F)c3)c2c1]

To a solution of 3-bromo-N-(3-(1-(difluoromethyl)-1H-pyrazol-4-yl)-1H-indazol-5-yl)-5,6,7,8-tetrahydroquinolin-8-amine (200 mg, 435.46 umol) in DMA (2 mL) was added zinc cyanide (102.27 mg, 870.91 umol), Zn (56.95 mg, 870.91 umol), 1,1-bis(diphenylphosphino)ferrocene (48.28 mg, 87.09 umol), and Pd₂dba₃ (79.75 mg, 87.09 umol) at 20° C. The mixture was stirred at 100° C. for 2 hrs under an N₂ atmosphere. The reaction mixture was concentrated. This procedure was conducted a second time using 100 mgs of the bromide starting material and the residues were combined. The material was purified by preparative-HPLC using Method BY to afford the title compound (50 mg, 19%) as a yellow solid. MS-ESI (m/z) calc'd for C₂₁H₁₈F₂N₇ [M+H]⁺: 406.2. Found 406.1.

### Step 7: 8-((3-(1-(Difluoromethyl)-1H-pyrazol-4-yl)-1H-indazol-5-yl)amino)-5,6,7,8-tetrahydroquinoline-3-carbonitrile, enantiomer 1 and 2

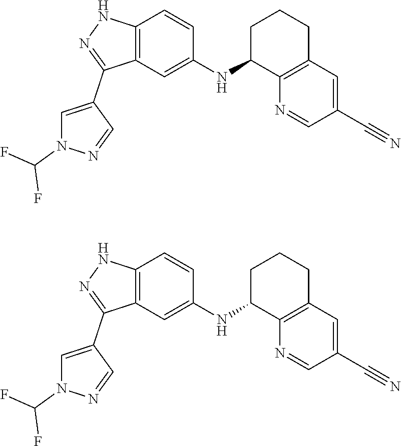 [SMILES: N#Cc1cnc2c(c1)CCC[C@@H]2Nc1ccc2[nH]nc(-c3cnn(C(F)F)c3)c2c1 ; N#Cc1cnc2c(c1)CCC[C@H]2Nc1ccc2[nH]nc(-c3cnn(C(F)F)c3)c2c1]

8-((3-(1-(Difluoromethyl)-1H-pyrazol-4-yl)-1H-indazol-5-yl)amino)-5,6,7,8-tetrahydroquinoline-3-carbonitrile was subjected to chiral separation using Method BZ to afford 8-((3-(1-(difluoromethyl)-1H-pyrazol-4-yl)-1H-indazol-5-yl)amino)-5,6,7,8-tetrahydroquinoline-3-carbonitrile, enantiomer 1 (2.03 mg, 22%) as a yellow oil: ¹H NMR (400 MHz, MeOD) δ 8.70 (d, J=1.76 Hz, 1H), 8.54 (s, 1H), 8.24 (s, 1H), 7.98 (s, 1H), 7.41-7.73 (m, 1H), 7.38 (d, J=8.82 Hz, 1H), 7.17 (s, 1H), 7.04-7.09 (m, 1H), 4.78 (t, J=5.07 Hz, 1H), 2.83-3.05 (m, 2H), 1.86-2.25 (m, 4H). MS-ESI (m/z) calc'd for C₂₁H₁₈F₂N₇ [M+H]⁺: 406.2. Found 406.0. A later eluting fraction was also isolated to afford 8-((3-(1-(difluoromethyl)-1H-pyrazol-4-yl)-1H-indazol-5-yl)amino)-5,6,7,8-tetrahydroquinoline-3-carbonitrile, enantiomer 2 (2.31 mg, 25%) as a yellow oil: ¹H NMR (400 MHz, MeOD) δ 8.70 (s, 1H), 8.54 (s, 1H), 8.24 (s, 1H), 7.98 (s, 1H), 7.40-7.74 (m, 1H), 7.38 (d, J=9.04 Hz, 1H), 7.17 (s, 1H), 7.07 (dd, J=9.04, 1.76 Hz, 1H), 4.78 (t, J=5.07 Hz, 1H), 2.81-3.03 (m, 2H), 1.87-2.23 (m, 4H). MS-ESI (m/z) calc'd for C₂₁H₁₈F₂N₇ [M+H]⁺: 406.2. Found 406.0.

### Example 50: 1-((3-(1-(Difluoromethyl)-1H-pyrazol-4-yl)-1H-indazol-5-yl)oxy)-4-methyl-2,3-dihydro-1H-indene-5-carbonitrile, enantiomer 1 and 2

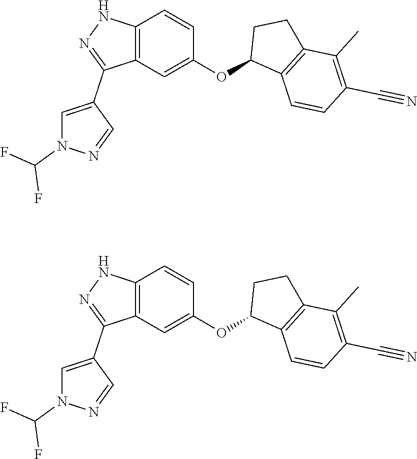 [SMILES: Cc1c(C#N)ccc2c1CC[C@@H]2Oc1ccc2[nH]nc(-c3cnn(C(F)F)c3)c2c1 ; Cc1c(C#N)ccc2c1CC[C@H]2Oc1ccc2[nH]nc(-c3cnn(C(F)F)c3)c2c1]

### Step 1: 5-Bromo-4-methyl-2,3-dihydro-1H-inden-1-ol

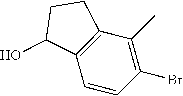

To a solution of 5-bromo-4-methyl-2,3-dihydro-1H-inden-1-one (200 mg, 888.57 umol) in EtOH (5 mL) was added NaBH₄ (67.23 mg, 1.78 mmol) at 0° C. and the mixture was stirred at 50° C. for 0.5 hr. The reaction mixture was quenched by addition of a saturated aqueous NaHCO₃ solution at 0° C. to give a final pH=8. The mixture was concentrated to give a residue that was diluted with H₂O and extracted with EtOAc (3×). The combined organic phases were dried over anhydrous Na₂SO₄, filtered, and the solvent evaporated to afford the title compound (200 mg, 99%) as a white solid. ¹H NMR (400 MHz, DMSO-d₆) δ 7.42 (d, J=7.88 Hz, 1H), 7.10 (d, J=8.00 Hz, 1H), 5.30 (br s, 1H), 5.02 (br t, J=6.38 Hz, 1H), 2.93 (ddd, J=16.10, 8.79, 3.88 Hz, 1H), 2.70 (dt, J=16.01, 7.88 Hz, 1H), 2.30-2.40 (m, 1H), 2.28 (s, 3H), 1.73-1.85 (m, 1H).

### Step 2: 1-Hydroxy-4-methyl-2,3-dihydro-1H-indene-5-carbonitrile

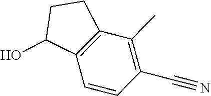

A mixture of 5-bromo-4-methyl-2,3-dihydro-1H-inden-1-ol (200 mg, 880.68 umol), zinc cyanide (155.12 mg, 1.32 mmol), Zn (5.76 mg, 88.07 umol), 1,1-bis(diphenylphosphino)ferrocene (48.82 mg, 88.07 umol) and Pd₂dba₃ (80.65 mg, 88.07 umol) in DMA (3 mL) was degassed and purged with N₂ (3×). The mixture was then stirred at 120° C. for 2 hrs under an N₂ atmosphere in a microwave reactor. The reaction was filtered and the filtrate was concentrated to give a residue. The residue was diluted with H₂O and extracted with EtOAc (3×). The combined organic phases were dried over anhydrous Na₂SO₄, filtered, and the solvent was evaporated to give a residue. The residue was purified by silica gel chromatography (ISCO; 4 g SepaFlash Column) using a 0-20% EtOAc/petroleum ether gradient eluent to afford the title compound (80 mg, 52%) as a white solid. MS-ESI (m/z) calc'd for C₁₁H₁₂NO [M+H]⁺: 174.1. Found 174.0.

### Step 3: 1-((3-(1-(Difluoromethyl)-1H-pyrazol-4-yl)-1H-indazol-5-yl)oxy)-4-methyl-2,3-dihydro-1H-indene-5-carbonitrile

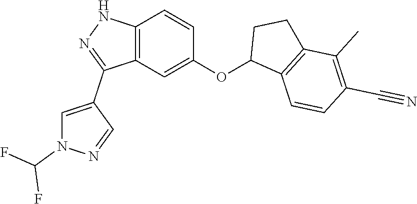 [SMILES: Cc1c(C#N)ccc2c1CCC2Oc1ccc2[nH]nc(-c3cnn(C(F)F)c3)c2c1]

To a solution of 1-hydroxy-4-methyl-2,3-dihydro-1H-indene-5-carbonitrile (60 mg, 346.40 umol) and 3-(1-(difluoromethyl)-1H-pyrazol-4-yl)-1H-indazol-5-ol (86.67 mg, 346.40 umol) in toluene (3 mL) was added tributylphosphine (140.17 mg, 692.80 umol) and 1,1-(azodicarbonyl)dipiperidine (174.80 mg, 692.80 umol) at 0° C. The mixture was then stirred at 100° C. for 12 hrs. The reaction was filtered and the filtrate was concentrated to give a residue that was purified by preparative-HPLC using Method BZ to afford the title compound (20 mg, 11%) as a brown solid. MS-ESI (m/z) calc'd for C₂₂H₁₈F₂N₅O [M+H]⁺: 406.1. Found 406.1.

### Step 4: 1-((3-(1-(Difluoromethyl)-1H-pyrazol-4-yl)-1H-indazol-5-yl)oxy)-4-methyl-2,3-dihydro-1H-indene-5-carbonitrile, enantiomer 1 and 2

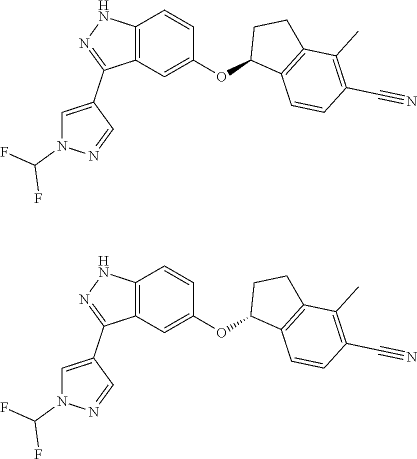 [SMILES: Cc1c(C#N)ccc2c1CC[C@@H]2Oc1ccc2[nH]nc(-c3cnn(C(F)F)c3)c2c1 ; Cc1c(C#N)ccc2c1CC[C@H]2Oc1ccc2[nH]nc(-c3cnn(C(F)F)c3)c2c1]

1-((3-(1-(Difluoromethyl)-1H-pyrazol-4-yl)-1H-indazol-5-yl)oxy)-4-methyl-2,3-dihydro-1H-indene-5-carbonitrile was subjected to chiral separation using Method CA to afford 1-((3-(1-(difluoromethyl)-1H-pyrazol-4-yl)-1H-indazol-5-yl)oxy)-4-methyl-2,3-dihydro-1H-indene-5-carbonitrile, enantiomer 1 (3.14 mg, 44%). ¹H NMR (400 MHz, DMSO-d₆) δ 13.08 (s, 1H), 8.91 (s, 1H), 8.38 (s, 1H), 7.70-8.06 (m, 1H), 7.60-7.69 (m, 2H), 7.51 (d, J=9.05 Hz, 1H), 7.42 (d, J=7.95 Hz, 1H), 7.12 (dd, J=8.93, 2.08 Hz, 1H), 6.14 (dd, J=6.60, 4.52 Hz, 1H), 2.87-3.12 (m, 2H), 2.60-2.75 (m, 1H), 2.47 (s, 3H), 2.09-2.20 (m, 1H). MS-ESI (m/z) calc'd for C₂₂H₁₈F₂N₅O [M+H]⁺: 406.1. Found 406.0. A later eluting fraction was also isolated to afford 1-((3-(1-(difluoromethyl)-1H-pyrazol-4-yl)-1H-indazol-5-yl)oxy)-4-methyl-2,3-dihydro-1H-indene-5-carbonitrile, enantiomer 2 (2.98 mg, 42%). ¹H NMR (400 MHz, DMSO-d₆) δ 13.08 (br s, 1H), 8.92 (s, 1H), 8.38 (s, 1H), 7.71-8.05 (m, 1H), 7.61-7.69 (m, 2H), 7.51 (d, J=8.93 Hz, 1H), 7.42 (d, J=7.82 Hz, 1H), 7.12 (dd, J=8.99, 2.14 Hz, 1H), 6.14 (dd, J=6.66, 4.59 Hz, 1H), 2.87-3.13 (m, 2H), 2.62-2.72 (m, 1H), 2.47 (s, 3H), 2.09-2.19 (m, 1H). MS-ESI (m/z) calc'd for C₂₂H₁₈F₂N₅O [M+H]⁺: 406.1. Found 406.0.

### Example 51: 1-((3-(1-(Difluoromethyl)-1H-pyrazol-4-yl)-1H-indazol-5-yl)oxy)-4-fluoro-2,3-dihydro-1H-indene-5-carbonitrile

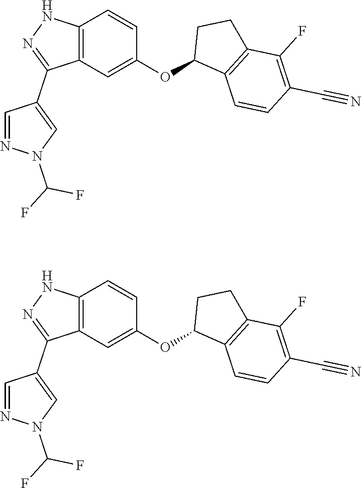 [SMILES: N#Cc1ccc2c(c1F)CC[C@@H]2Oc1ccc2[nH]nc(-c3cnn(C(F)F)c3)c2c1 ; N#Cc1ccc2c(c1F)CC[C@H]2Oc1ccc2[nH]nc(-c3cnn(C(F)F)c3)c2c1]

### Step 1: 5-Bromo-4-fluoro-2,3-dihydro-1H-inden-1-ol

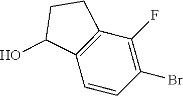 [SMILES: OC1CCc2c1ccc(Br)c2F]

To a solution of 5-bromo-4-fluoro-2,3-dihydro-1H-inden-1-one (200 mg, 873.19 umol) in EtOH (5 mL) was added NaBH₄ (66.07 mg, 1.75 mmol) at 20° C. The mixture was stirred at 60° C. for 0.1 hr. The reaction mixture was diluted with H₂O and extracted with EtOAc (3×). The combined organic phases were dried over Na₂SO₄, filtered, and concentrated under reduced pressure to afford the title compound (180 mg, 85%) as a yellow solid. ¹H NMR (400 MHz, CDCl₃) δ 7.38-7.48 (m, 1H), 7.08 (d, J=8.16 Hz, 1H), 5.24 (br t, J=6.06 Hz, 1H), 3.13 (ddd, J=4.63, 8.65, 16.48 Hz, 1H), 2.78-2.94 (m, 1H), 2.48-2.62 (m, 1H), 1.93-2.06 (m, 1H), 1.84 (br s, 1H).

### Step 2: 4-Fluoro-1-hydroxy-2,3-dihydro-1H-indene-5-carbonitrile

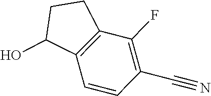 [SMILES: N#Cc1ccc2c(c1F)CCC2O]

A mixture of 5-bromo-4-fluoro-2,3-dihydro-1H-inden-1-ol (180 mg, 779.01 umol), Zn(CN)₂ (137.21 mg, 1.17 mmol), Pd₂dba₃ (71.34 mg, 77.90 umol), 1,1-bis(diphenylphosphino)ferrocene (43.19 mg, 77.90 umol) and Zn (5.09 mg, 77.90 umol) in DMA (4 mL) was degassed and purged with N₂ (3×) at 20° C. The mixture was then stirred at 120° C. for 2 hrs under an N₂ atmosphere in a microwave reactor. The reaction mixture was diluted with H₂O and extracted with EtOAc (3×). The combined organic phases were dried over Na₂SO₄ and concentrated to give a residue. The residue was purified by preparative-TLC (SiO₂, petroleum ether/EtOAc=1/1) to afford the title compound (68 mg, 49%) as a yellow solid. ¹H NMR (400 MHz, CDCl₃) δ 7.53 (dd, J=5.66, 7.69 Hz, 1H), 7.31 (d, J=7.75 Hz, 1H), 5.31 (t, J=6.68 Hz, 1H), 3.15 (ddd, J=3.93, 8.79, 16.60 Hz, 1H), 2.80-2.96 (m, 1H), 2.62 (dddd, J=3.99, 7.12, 8.21, 13.34 Hz, 1H), 1.97-2.14 (m, 1H). MS-ESI (m/z) calc'd for C₁₀H₇FNO [M−H]⁻: 176.1. Found 175.9.

### Step 3: 5-Bromo-3-(1-(difluoromethyl)-1H-pyrazol-4-yl)-1H-indazole

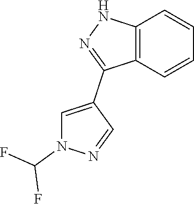 [SMILES: FC(F)n1cc(-c2n[nH]c3ccccc23)cn1]

A mixture of 5-bromo-3-iodo-1H-indazole (800 mg, 2.47 mmol), 1-(difluoromethyl)-4-(4,4,5,5-tetramethyl-1,3,2-dioxaborolan-2-yl)-1H-pyrazole (925.76 mg, 3.79 mmol), Pd(dppf)Cl₂ (181.27 mg, 244.73 umol), K₂CO₃ (1.03 g, 7.43 mmol) in H₂O (3 mL) and dioxane (30 mL) was degassed and purged with N₂ (3×) at 20° C. The mixture was then stirred at 110° C. for 2 hrs under an N₂ atmosphere. The reaction mixture was concentrated to give a residue that was purified by silica gel chromatography (ISCO; 25 g SepaFlash column) using a 0-20% EtOAc/petroleum ether gradient eluent to afford the title compound (750 mg, 95%) as a white solid. ¹H NMR (400 MHz, DMSO-d₆) δ 13.36 (br s, 1H), 9.02 (s, 1H), 8.35 (d, J=5.07 Hz, 2H), 7.67-8.02 (m, 1H), 7.49-7.58 (m, 2H). MS-ESI (m/z) calc'd for C₁₁H₈BrF₂N₄ [M+H]⁺: 313.0/315.0. Found 312.9/314.9.

### Step 4: 3-(1-(Difluoromethyl)-1H-pyrazol-4-yl)-5-(4,4,5,5-tetramethyl-1,3,2-dioxaborolan-2-yl)-1H-indazole

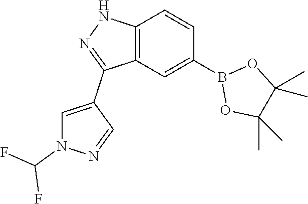 [SMILES: CC1(C)OB(c2ccc3[nH]nc(-c4cnn(C(F)F)c4)c3c2)OC1(C)C]

A mixture of 5-bromo-3-(1-(difluoromethyl)-1H-pyrazol-4-yl)-1H-indazole (740 mg, 2.36 mmol), 4,4,5,5-tetramethyl-2-(4,4,5,5-tetramethyl-1,3,2-dioxaborolan-2-yl)-1,3,2-dioxaborolane (660.19 mg, 2.60 mmol), Pd(dppf)Cl₂ (172.94 mg, 236.35 umol), KOAc (695.87 mg, 7.09 mmol) in 1,4-dioxane (20 mL) was degassed and purged with N₂ (3×) at 20° C. The mixture was then stirred at 120° C. for 2 hrs under an N₂ atmosphere. The reaction mixture was concentrated under reduced pressure to remove solvent and the residue was purified by silica gel chromatography (ISCO; 12 g SepaFlash column) using a 0-19% EtOAc/petroleum ether gradient eluent to afford the title compound (580 mg, 68%) as a yellow oil. ¹H NMR (400 MHz, CDCl₃) δ 8.39 (d, J=18.96 Hz, 2H), 8.28 (s, 1H), 7.81-7.90 (m, 1H), 7.47 (d, J=8.82 Hz, 1H), 7.12-7.33 (m, 1H), 1.23-1.25 (m, 12H). MS-ESI (m/z) calc'd for C₁₇H₂₀BF₂N₄O₂ [M+H]⁺: 361.2. Found 361.1.

### Step 5: 3-(1-(Difluoromethyl)-1H-pyrazol-4-yl)-1H-indazol-5-ol

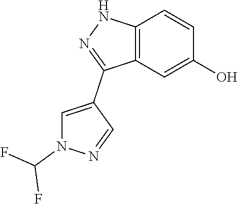 [SMILES: Oc1ccc2[nH]nc(-c3cnn(C(F)F)c3)c2c1]

To a solution of 3-(1-(difluoromethyl)-1H-pyrazol-4-yl)-5-(4,4,5,5-tetramethyl-1,3,2-dioxaborolan-2-yl)-1H-indazole (460 mg, 1.28 mmol) in THF (6 mL) and H₂O (6 mL) was added sodium perborate tetrahydrate (589.53 mg, 3.83 mmol) at 20° C. The mixture was stirred at 50° C. for 1 hr. The reaction mixture was filtered and the solid was dried under vacuum to afford the product. The filtrate was also worked up to recover product, the filtrate was diluted with H₂O and extracted with EtOAc (3×). The organic phase was dried over Na₂SO₄, filtered and concentrated under reduced pressure to give a residue. The residue was purified by silica gel chromatography (ISCO; 4 g SepaFlash column) using a 0-20% EtOAc/petroleum ether gradient eluent. This was combined with the initial solid collected to afford the title compound (290 mg, 90%) as a white solid. ¹H NMR (400 MHz, DMSO-d₆) δ 12.91 (s, 1H), 9.20 (s, 1H), 8.72 (s, 1H), 8.25 (s, 1H), 7.69-8.05 (m, 1H), 7.39 (d, J=8.82 Hz, 1H), 7.22 (d, J=2.03 Hz, 1H), 6.97 (dd, J=2.15, 8.94 Hz, 1H). MS-ESI (m/z) calc'd for C₁₁H₉F₂N₄O [M+H]⁺: 251.1. Found 251.0.

### Step 6: 1-((3-(1-(Difluoromethyl)-1H-pyrazol-4-yl)-1H-indazol-5-yl)oxy)-4-fluoro-2,3-dihydro-1H-indene-5-carbonitrile

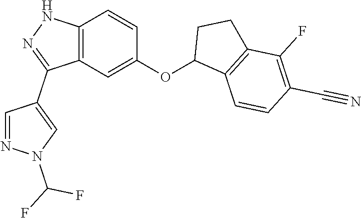 [SMILES: N#Cc1ccc2c(c1F)CCC2Oc1ccc2[nH]nc(-c3cnn(C(F)F)c3)c2c1]

To a solution of 4-fluoro-1-hydroxy-2,3-dihydro-1H-indene-5-carbonitrile (50 mg, 282.21 umol), 3-(1-(difluoromethyl)-1H-pyrazol-4-yl)-1H-indazol-5-ol (70.61 mg, 282.21 umol) in toluene (4 mL) was added tributylphosphine (114.19 mg, 564.41 umol) and N-(piperidine-1-carbonylimino)piperidine-1-carboxamide (142.41 mg, 564.41 umol) at 0° C. The mixture was then stirred at 100° C. for 12 hrs. The reaction mixture was filtered and the filtrate was concentrated to give a residue that was purified by preparative-HPLC using Method CB to afford the title compound (20 mg, 17%) as a brown solid. MS-ESI (m/z) calc'd for C₂₁H₁₅F₃N₅O [M+H]⁺: 410.1. Found 410.1.

### Step 7: 1-((3-(1-(Difluoromethyl)-1H-pyrazol-4-yl)-1H-indazol-5-yl)oxy)-4-fluoro-2,3-dihydro-1H-indene-5-carbonitrile

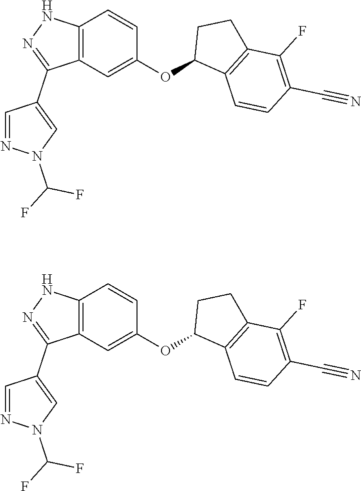 [SMILES: N#Cc1ccc2c(c1F)CC[C@@H]2Oc1ccc2[nH]nc(-c3cnn(C(F)F)c3)c2c1 ; N#Cc1ccc2c(c1F)CC[C@H]2Oc1ccc2[nH]nc(-c3cnn(C(F)F)c3)c2c1]

1-((3-(1-(Difluoromethyl)-1H-pyrazol-4-yl)-1H-indazol-5-yl)oxy)-4-fluoro-2,3-dihydro-1H-indene-5-carbonitrile was subjected to chiral separation using Method CC to afford 1-((3-(1-(difluoromethyl)-1H-pyrazol-4-yl)-1H-indazol-5-yl)oxy)-4-fluoro-2,3-dihydro-1H-indene-5-carbonitrile, enantiomer 1 (2.92 mg, 32%). ¹H NMR (400 MHz, DMSO-d₆) δ 13.11 (br s, 1H), 8.92 (s, 1H), 8.38 (s, 1H), 7.72-8.04 (m, 2H), 7.65 (d, J=1.96 Hz, 1H), 7.41-7.54 (m, 2H), 7.13 (dd, J=8.99, 2.14 Hz, 1H), 6.15-6.24 (m, 1H), 2.97-3.23 (m, 2H), 2.73 (td, J=13.30, 7.64 Hz, 1H), 2.13-2.25 (m, 1H). MS-ESI (m/z) calc'd for C₂₁H₁₅F₃N₅O [M+H]⁺: 410.1. Found 410.0. A later eluting fraction was also isolated to afford 1-((3-(1-(difluoromethyl)-1H-pyrazol-4-yl)-1H-indazol-5-yl)oxy)-4-fluoro-2,3-dihydro-1H-indene-5-carbonitrile, enantiomer 2 (2.58 mg, 29%). ¹H NMR (400 MHz, DMSO-d₆) δ 8.92 (s, 1H), 8.38 (s, 1H), 7.72-8.04 (m, 2H), 7.65 (d, J=1.71 Hz, 1H), 7.53 (br d, J=8.80 Hz, 1H), 7.45 (d, J=7.82 Hz, 1H), 7.13 (dd, J=8.93, 2.08 Hz, 1H), 6.19 (t, J=5.62 Hz, 1H), 2.94-3.26 (m, 2H), 2.73 (td, J=13.17, 7.64 Hz, 1H), 2.15-2.25 (m, 1H). MS-ESI (m/z) calc'd for C₂₁H₁₅F₃N₅O [M+H]⁺: 410.1. Found 410.0.

### Example 52: 1-((3-(1-(Difluoromethyl)-1H-pyrazol-4-yl)-1H-indazol-5-yl)amino)-4-methyl-2,3-dihydro-1H-indene-5-carbonitrile, enantiomer 1 and 2

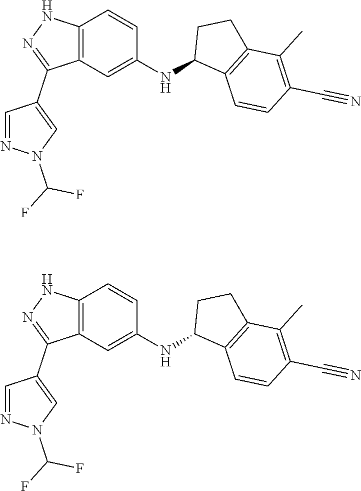 [SMILES: Cc1c(C#N)ccc2c1CC[C@@H]2Nc1ccc2[nH]nc(-c3cnn(C(F)F)c3)c2c1 ; Cc1c(C#N)ccc2c1CC[C@H]2Nc1ccc2[nH]nc(-c3cnn(C(F)F)c3)c2c1]

### Step 1: 3-Bromo-5-nitro-1H-indazole

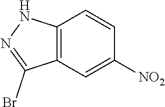 [SMILES: O=[N+]([O-])c1ccc2[nH]nc(Br)c2c1]

To a solution of 5-nitro-1H-indazole (10 g, 61.30 mmol) in AcOH (200 mL) was added bromine (15.80 mL, 306.50 mmol) at 20° C. The mixture was then stirred at 80° C. for 2 hrs. The reaction mixture was poured into H₂O, filtered and the solid was dried under vacuum to afford the title compound (13.09 g, 70%) as a pale yellow solid.

### Step 2: 3-(1-(Difluoromethyl)-1H-pyrazol-4-yl)-5-nitro-1H-indazole

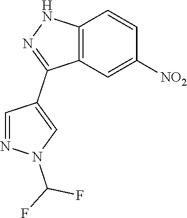 [SMILES: O=[N+]([O-])c1ccc2[nH]nc(-c3cnn(C(F)F)c3)c2c1]

A mixture of 3-bromo-5-nitro-1H-indazole (1 g, 4.13 mmol), 1-(difluoromethyl)-4-(4,4,5,5-tetramethyl-1,3,2-dioxaborolan-2-yl)pyrazole (1.01 g, 4.13 mmol), AcOK (1.22 g, 12.40 mmol) and bis(4-(di-tert-butylphosphanyl)-N,N-dimethylaniline); dichloropalladium (292.56 mg, 413.17 umol) in EtOH (20 mL) and H₂O (4 mL) was degassed and purged with N₂ (3×) at 20° C., and then the mixture was stirred at 90° C. for 12 hrs under an N₂ atmosphere. The reaction was filtered and the filtrate was evaporated to give a residue that was diluted with H₂O and extracted with EtOAc (3×). The combined organic phases were dried over anhydrous Na₂SO₄ and evaporated. The material was purified by flash silica gel column chromatography (ISCO; 12 g SepaFlash column) using a 0-20% EtOAc/petroleum ether gradient eluent to afford the title compound (1.09 g, 47%) as a yellow solid. MS-ESI (m/z) calc'd for C₁₁H₈F₂N₅O₂ [M+H]⁺: 280.1. Found 280.2.

### Step 3: 3-(1-(Difluoromethyl)-1H-pyrazol-4-yl)-1H-indazol-5-amine

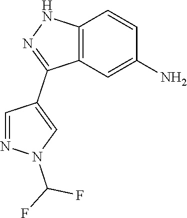 [SMILES: Nc1ccc2[nH]nc(-c3cnn(C(F)F)c3)c2c1]

To a solution of 3-(1-(difluoromethyl)-1H-pyrazol-4-yl)-5-nitro-1H-indazole (1.09 g, 3.89 mmol) in EtOH (8 mL) and H₂O (8 mL) was added Fe (1.09 g, 19.47 mmol) and NH₄Cl (1.04 g, 19.47 mmol) at 20° C. The mixture was then stirred at 80° C. for 2 hrs and filtered. The filtrate was evaporated to dryness to afford the title compound (756 mg, 69%) as a red solid. MS-ESI (m/z) calc'd for C₁₁H₁₀F₂N₅ [M+H]⁺: 250.1. Found 250.1.

### Step 4: N-(5-Bromo-4-methyl-2,3-dihydro-1H-inden-1-yl)-3-(1-(difluoromethyl)-1H-pyrazol-4-yl)-1H-indazol-5-amine

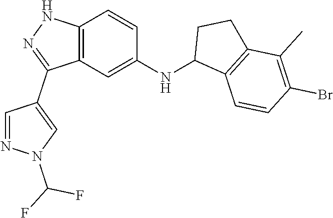 [SMILES: Cc1c(Br)ccc2c1CCC2Nc1ccc2[nH]nc(-c3cnn(C(F)F)c3)c2c1]

To a solution of 3-(1-(difluoromethyl)-1H-pyrazol-4-yl)-1H-indazol-5-amine (177.16 mg, 710.85 umol) and 5-bromo-4-methyl-indan-1-one (80 mg, 355.43 umol) in toluene (4 mL) was added Ti(i-PrO)₄ (505.09 mg, 1.78 mmol) at 20° C. The mixture was then stirred at 130° C. for 6 hrs. The reaction mixture was concentrated under reduced pressure to remove solvent. The residue was diluted with MeOH (4 mL) and then NaBH₄ (107.57 mg, 2.84 mmol) was added to the mixture at 0° C. and the mixture was stirred at 20° C. for 4 hrs. The mixture was filtered and the filtrate was concentrated to give a residue that was purified by flash silica gel column chromatography (ISCO; 12 g SepaFlash column) using a 0-23% EtOAc/petroleum ether gradient eluent to afford the title compound (165 mg, 23%) as a yellow oil. MS-ESI (m/z) calc'd for C₂₁H₁₉BrF₂N₅ [M+H]⁺: 458.1/460.1. Found 458.1/460.1.

### Step 5: 1-((3-(1-(Difluoromethyl)-1H-pyrazol-4-yl)-1H-indazol-5-yl)amino)-4-methyl-2,3-dihydro-1H-indene-5-carbonitrile

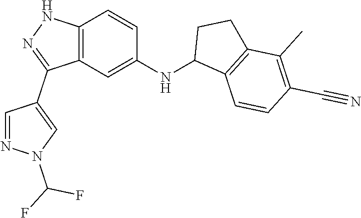 [SMILES: Cc1c(C#N)ccc2c1CCC2Nc1ccc2[nH]nc(-c3cnn(C(F)F)c3)c2c1]

N-(5-Bromo-4-methyl-2,3-dihydro-1H-inden-1-yl)-3-(1-(difluoromethyl)-1H-pyrazol-4-yl)-1H-indazol-5-amine (145 mg, 316.39 umol), Zn(CN)₂ (111.45 mg, 949.16 umol), Zn (20.69 mg, 316.39 umol), 1,1-bis(diphenylphosphino)ferrocene (52.62 mg, 94.92 umol) and Pd₂dba₃ (28.97 mg, 31.64 umol) were placed in a microwave reaction tube in DMA (5 mL) at 20° C. under an N₂ atmosphere. The sealed tube was heated at 100° C. for 2 hrs under microwave irradiation. The mixture was filtered and the filtrate was purified by preparative-HPLC using Method CD to afford the title compound (50 mg, 30%) as a yellow solid. MS-ESI (m/z) calc'd for C₂₂H₁₉F₂N₆ [M+H]⁺: 405.2. Found 405.1.

### Step 6: 1-((3-(1-(Difluoromethyl)-1H-pyrazol-4-yl)-1H-indazol-5-yl)amino)-4-methyl-2,3-dihydro-1H-indene-5-carbonitrile, enantiomer 1 and 2

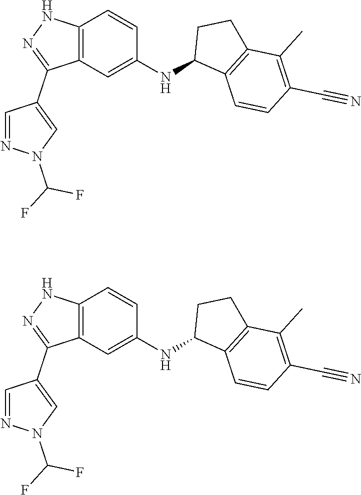 [SMILES: Cc1c(C#N)ccc2c1CC[C@@H]2Nc1ccc2[nH]nc(-c3cnn(C(F)F)c3)c2c1 ; Cc1c(C#N)ccc2c1CC[C@H]2Nc1ccc2[nH]nc(-c3cnn(C(F)F)c3)c2c1]

1-((3-(1-(Difluoromethyl)-1H-pyrazol-4-yl)-1H-indazol-5-yl)amino)-4-methyl-2,3-dihydro-1H-indene-5-carbonitrile was subjected to chiral separation using Method CE to afford 1-((3-(1-(difluoromethyl)-1H-pyrazol-4-yl)-1H-indazol-5-yl)amino)-4-methyl-2,3-dihydro-1H-indene-5-carbonitrile, enantiomer 1 (3.46 mg, 38%) as a yellow solid. ¹H NMR (400 MHz, DMSO-d₆) δ 12.80 (br s, 1H), 8.71 (s, 1H), 8.28 (s, 1H), 8.03-7.67 (m, 1H), 7.58 (d, J=7.9 Hz, 1H), 7.40-7.23 (m, 2H), 7.10 (s, 1H), 6.96 (dd, J=1.9, 8.9 Hz, 1H), 5.84 (d, J=9.3 Hz, 1H), 5.31 (q, J=7.8 Hz, 1H), 3.05-2.96 (m, 1H), 2.84 (td, J=8.0, 16.5 Hz, 1H), 2.68-2.57 (m, 1H), 2.44 (s, 3H), 1.92-1.81 (m, 1H). MS-ESI (m/z) calc'd for C₂₂H₁₉F₂N₆ [M+H]⁺: 405.2. Found 405.2. A later eluting fraction was also isolated to afford 1-((3-(1-(difluoromethyl)-1H-pyrazol-4-yl)-1H-indazol-5-yl)amino)-4-methyl-2,3-dihydro-1H-indene-5-carbonitrile, enantiomer 2 (2.84 mg, 31%) as a yellow solid. ¹H NMR (400 MHz, DMSO-d₆) δ 12.80 (br s, 1H), 8.71 (s, 1H), 8.28 (s, 1H), 8.01-7.69 (m, 1H), 7.58 (d, J=7.9 Hz, 1H), 7.37-7.26 (m, 2H), 7.10 (s, 1H), 6.96 (dd, J=2.0, 9.0 Hz, 1H), 5.84 (d, J=8.6 Hz, 1H), 5.35-5.26 (m, 1H), 3.07-2.96 (m, 1H), 2.84 (td, J=8.5, 16.4 Hz, 1H), 2.68-2.59 (m, 1H), 2.44 (s, 3H), 1.92-1.81 (m, 1H). MS-ESI (m/z) calc'd for C₂₂H₁₉F₂N₆ [M+H]⁺: 405.2. Found 405.2.

### Example 53: 1-((3-(1-(Difluoromethyl)-1H-pyrazol-4-yl)-1H-indazol-5-yl)amino)-4-fluoro-2,3-dihydro-1H-indene-5-carbonitrile, enantiomer 1 and 2

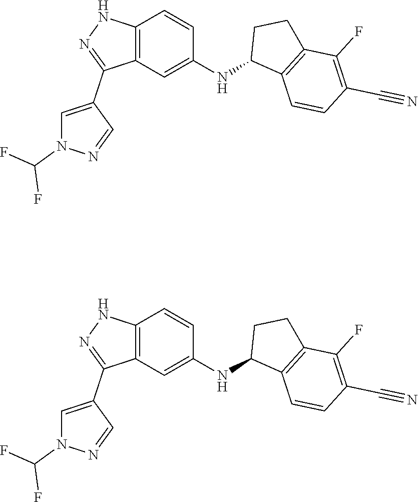 [SMILES: N#Cc1ccc2c(c1F)CC[C@H]2Nc1ccc2[nH]nc(-c3cnn(C(F)F)c3)c2c1 ; N#Cc1ccc2c(c1F)CC[C@@H]2Nc1ccc2[nH]nc(-c3cnn(C(F)F)c3)c2c1]

### Step 1: 3-(1-(Difluoromethyl)-1H-pyrazol-4-yl)-5-nitro-1H-indazole

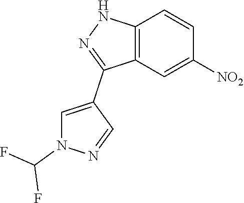 [SMILES: O=[N+]([O-])c1ccc2[nH]nc(-c3cnn(C(F)F)c3)c2c1]

A mixture of 3-bromo-5-nitro-1H-indazole (500 mg, 2.07 mmol), 1-(difluoromethyl)-4-(4,4,5,5-tetramethyl-1,3,2-dioxaborolan-2-yl)pyrazole (504.16 mg, 2.07 mmol), KOAc (608.23 mg, 6.20 mmol), bis(4-(di-tert-butylphosphanyl)-N,N-dimethylaniline); dichloropalladium (146.28 mg, 206.59 umol) in EtOH (10 mL) and H₂O (2 mL) was degassed and purged with N₂ (3×) at 20° C., then the mixture was stirred at 90° C. for 12 hrs under an N₂ atmosphere. The reaction mixture was evaporated to give a residue which was diluted with H₂O and extracted with EtOAc (3×). The combined organic phases were dried over anhydrous Na₂SO₄, filtered, and the solvent was evaporated. The material was purified by flash silica gel chromatography (ISCO; 4 g SepaFlash column) using a 0-30% EtOAc/petroleum ether gradient eluent to afford the title compound (260 mg, 56%) as a yellow solid. MS-ESI (m/z) calc'd for C₁₁H₈F₂N₅O₂ [M+H]⁺: 280.1. Found 280.0.

### Step 2: 3-(1-(Difluoromethyl)-1H-pyrazol-4-yl)-1H-indazol-5-amine

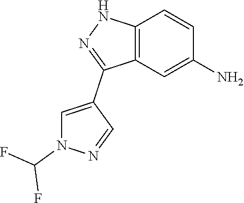 [SMILES: Nc1ccc2[nH]nc(-c3cnn(C(F)F)c3)c2c1]

To a solution of 3-(1-(difluoromethyl)-1H-pyrazol-4-yl)-5-nitro-1H-indazole (260 mg, 931.23 umol) in EtOH (2 mL) and H₂O (2 mL) was added Fe (260.02 mg, 4.66 mmol) and NH₄Cl (249.06 mg, 4.66 mmol) at 20° C. The mixture was stirred at 80° C. for 2 hrs. The reaction mixture was filtered and the filtrate was evaporated to give a residue. The residue was diluted with H₂O and extracted with EtOAc (3×). The combined organic layers were passed through a phase separator and concentrated to afford the title compound (180 mg, 78%) as a yellow solid. MS-ESI (m/z) calc'd for C₁₁H₁₀F₂N₅ [M+H]⁺: 250.1. Found 250.0.

### Step 3: N-(5-Bromo-4-fluoro-2,3-dihydro-1H-inden-1-ylidene)-3-(1-(difluoromethyl)-1H-pyrazol-4-yl)-1H-indazol-5-amine

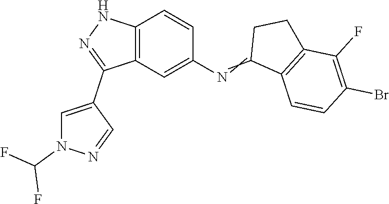 [SMILES: Fc1c(Br)ccc2c1CCC2=Nc1ccc2[nH]nc(-c3cnn(C(F)F)c3)c2c1]

To a solution of 3-[1-(difluoromethyl)pyrazol-4-yl]-1H-indazol-5-amine (150 mg, 601.88 umol) and 5-bromo-4-fluoro-indan-1-one (137.86 mg, 601.88 umol) in toluene (4 mL) was added Ti(i-PrO)₄ (855.30 mg, 3.01 mmol) and the mixture was stirred at 120° C. for 12 hrs. The solvent was evaporated from the reaction mixture to afford the title compound (260 mg, 94%) as a yellow solid which was used without further purification. MS-ESI (m/z) calc'd for C₂₀H₁₄BrF₃N₅ [M+H]⁺: 460.0/462.0. Found 460.0/462.0.

### Step 4: N-(5-Bromo-4-fluoro-2,3-dihydro-1H-inden-1-yl)-3-(1-(difluoromethyl)-1H-pyrazol-4-yl)-1H-indazol-5-amine

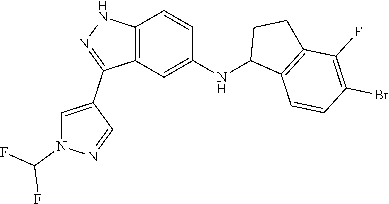 [SMILES: Fc1c(Br)ccc2c1CCC2Nc1ccc2[nH]nc(-c3cnn(C(F)F)c3)c2c1]

To a solution of N-(5-bromo-4-fluoro-2,3-dihydro-1H-inden-1-ylidene)-3-(1-(difluoromethyl)-1H-pyrazol-4-yl)-1H-indazol-5-amine (290.99 mg, 632.25 umol) in MeOH (5 mL) was added NaBH₄ (191.36 mg, 5.06 mmol) at 0° C. The mixture was stirred at 20° C. for 12 hrs. The solvent was evaporated from the reaction mixture to give a residue which was purified by preparative-TLC (petroleum ether/EtOAc=1/1, Rf=0.33) to afford the title compound (140 mg, 48%) as a white solid. MS-ESI (m/z) calc'd for C₂₀H₁₆BrF₃N₅ [M+H]⁺: 462.1/464.1. Found 462.1/464.0.

### Step 5: 1-((3-(1-(Difluoromethyl)-1H-pyrazol-4-yl)-1H-indazol-5-yl)amino)-4-fluoro-2,3-dihydro-1H-indene-5-carbonitrile

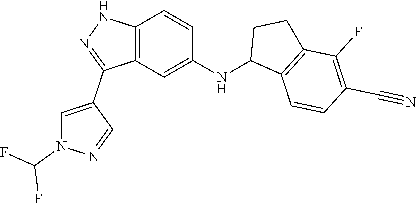 [SMILES: N#Cc1ccc2c(c1F)CCC2Nc1ccc2[nH]nc(-c3cnn(C(F)F)c3)c2c1]

A mixture of N-(5-bromo-4-fluoro-indan-1-yl)-3-[1-(difluoromethyl)pyrazol-4-yl]-1H-indazol-5-amine (140 mg, 302.86 umol), zinc cyanide (106.69 mg, 908.57 umol), Zn (19.80 mg, 302.86 umol), 1,1-bis(diphenylphosphino)ferrocene (50.37 mg, 90.86 umol) and Pd₂dba₃ (27.73 mg, 30.29 umol) in DMA (3 mL) was degassed and purged with N₂ (3×). The mixture was then stirred at 100° C. for 2 hrs under an N₂ atmosphere in a microwave reactor. The reaction mixture was filtered and the filtrate was concentrated to give a residue which was purified by preparative-TLC (petroleum ether/EtOAc=1/1, Rf=0.33) and further purified by preparative-HPLC using Method CF to afford the title compound (56 mg, 45%) as white solid. MS-ESI (m/z) calc'd for C₂₁H₁₆F₃N₆ [M+H]⁺: 409.1. Found 409.1.

### Step 6: 1-((3-(1-(Difluoromethyl)-1H-pyrazol-4-yl)-1H-indazol-5-yl)amino)-4-fluoro-2,3-dihydro-1H-indene-5-carbonitrile, enantiomer 1 and 2

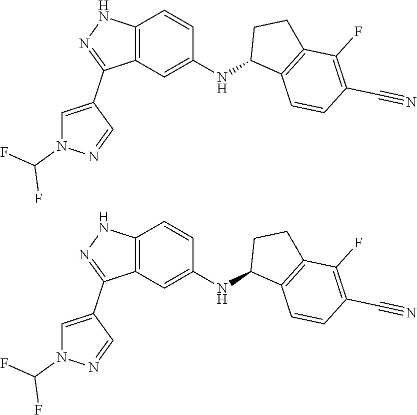 [SMILES: N#Cc1ccc2c(c1F)CC[C@H]2Nc1ccc2[nH]nc(-c3cnn(C(F)F)c3)c2c1 ; N#Cc1ccc2c(c1F)CC[C@@H]2Nc1ccc2[nH]nc(-c3cnn(C(F)F)c3)c2c1]

1-((3-(1-(Difluoromethyl)-1H-pyrazol-4-yl)-1H-indazol-5-yl)amino)-4-fluoro-2,3-dihydro-1H-indene-5-carbonitrile was subjected to chiral separation using Method CG to afford 1-((3-(1-(difluoromethyl)-1H-pyrazol-4-yl)-1H-indazol-5-yl)amino)-4-fluoro-2,3-dihydro-1H-indene-5-carbonitrile, enantiomer 1 (3.65 mg, 40%) as a gray solid. ¹H NMR (400 MHz, DMSO-d₆) δ 12.82 (s, 1H), 8.73 (s, 1H), 8.30 (s, 1H), 7.69-8.02 (m, 2H), 7.27-7.38 (m, 2H), 7.15 (s, 1H), 6.97 (dd, J=9, 2 Hz, 1H), 5.94 (d, J=9 Hz, 1H), 5.36-5.45 (m, 1H), 3.08-3.19 (m, 1H), 2.93 (dt, J=16, 8 Hz, 1H), 2.63-2.72 (m, 1H), 1.94 (dq, J=13, 8 Hz, 1H). MS-ESI (m/z) calc'd for C₂₁H₁₆F₃N₆ [M+H]⁺: 409.1. Found 409.0. A later eluting fraction was also isolated to afford 1-((3-(1-(difluoromethyl)-1H-pyrazol-4-yl)-1H-indazol-5-yl)amino)-4-fluoro-2,3-dihydro-1H-indene-5-carbonitrile, enantiomer 2 (4.33 mg, 46%) as a gray solid. ¹H NMR (400 MHz, DMSO-d₆) δ 12.82 (s, 1H), 8.73 (s, 1H), 8.30 (s, 1H), 7.67-8.03 (m, 2H), 7.26-7.39 (m, 2H), 7.15 (s, 1H), 6.97 (dd, J=9, 2 Hz, 1H), 5.94 (br d, J=9 Hz, 1H), 5.40 (q, J=8 Hz, 1H), 3.11 (ddd, J=16, 9, 3 Hz, 1H), 2.93 (dt, J=16, 8 Hz, 1H) 2.62-2.72 (m, 1H), 1.94 (dq, J=13, 8 Hz, 1H). MS-ESI (m/z) calc'd for C₂₁H₁₆F₃N₆ [M+H]⁺: 409.1. Found 409.0.

### Example 54: 5-((3-(Cyclohex-1-en-1-yl)-1H-indazol-5-yl)amino)-5,6,7,8-tetrahydronaphthalene-2-carbonitrile

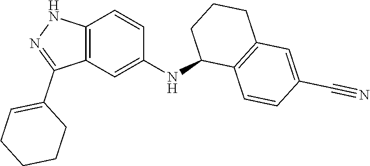 [SMILES: N#Cc1ccc2c(c1)CCC[C@@H]2Nc1ccc2[nH]nc(C3=CCCCC3)c2c1]

### Step 1: 3-Iodo-5-nitro-1H-indazole

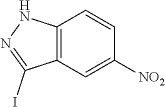 [SMILES: O=[N+]([O-])c1ccc2[nH]nc(I)c2c1]

To a solution of 5-nitro-1H-indazole (5 g, 30.65 mmol) in DMF (50 mL) was added iodine (23.34 g, 91.95 mmol) and KOH (6.54 g, 116.47 mmol) at 25° C. The mixture was stirred at 65° C. for 2 hrs. The reaction mixture was then poured into saturated aqueous Na₂SO₃ and a yellow solid formed. The solid was collected by filtration, washed with H₂O (3×), and dried under vacuum to afford the title compound (9.57 g, 60%) as a yellow solid.

### Step 2: 3-Iodo-1H-indazol-5-amine

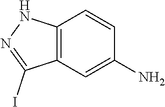 [SMILES: Nc1ccc2[nH]nc(I)c2c1]

To a solution of 3-iodo-5-nitro-1H-indazole (9.57 g, 33.11 mmol) in EtOH (216 mL) and H₂O (72 mL) was added NH₄Cl (8.86 g, 165.55 mmol) and Fe (9.25 g, 165.55 mmol) at 25° C. The mixture was then stirred at 80° C. for 1 hr and filtered. The filtrate was evaporated to give a residue that was diluted with H₂O and extracted with EtOAc (3×). The combined organic phases were dried over anhydrous Na₂SO₄, filtered, and the filtrate was concentrated to give a residue. The residue was purified by flash silica gel column chromatography (ISCO; 40 g SepaFlash column) using a 0-42% EtOAc/petroleum ether gradient eluent to afford the title compound (4 g, 53%) as a yellow solid. ¹H NMR (400 MHz, DMSO-d₆) δ 13.03 (s, 1H), 7.25 (d, J=8.8 Hz, 1H), 6.84 (dd, J=2.0, 8.8 Hz, 1H), 6.43 (d, J=1.3 Hz, 1H), 5.00 (s, 2H). MS-ESI (m/z) calc'd for C₇H₇IN₃ [M+H]⁺: 260.0. Found 259.9.

### Step 3: 5-((3-Iodo-1H-indazol-5-yl)amino)-5,6,7,8-tetrahydronaphthalene-2-carbonitrile

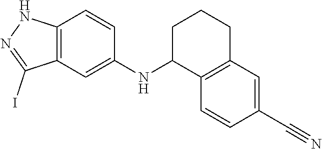 [SMILES: N#Cc1ccc2c(c1)CCCC2Nc1ccc2[nH]nc(I)c2c1]

To a solution of 3-iodo-1H-indazol-5-amine (2.46 g, 9.50 mmol) and 1-oxotetralin-6-carbonitrile (1.02 g, 5.94 mmol) in toluene (32 mL) was added Ti(Oi-Pr)₄ (8.76 mL, 29.69 mmol) at 20° C. The mixture was then stirred at 110° C. for 3 hrs. The reaction mixture was concentrated to give a residue that was diluted with MeOH (32 mL). NaBH₄ (1.80 g, 47.51 mmol) was then added at 0° C. and the mixture was stirred at 20° C. for 12 hrs. The mixture was filtered and the filtrate was evaporated to give a residue which was purified by flash silica gel column chromatography (ISCO; 20 g SepaFlash column) using a 0-100% EtOAc/petroleum ether gradient eluent to afford the title compound (1 g, 33%) as a yellow solid. MS-ESI (m/z) calc'd for C₁₈H₁₆IN₄ [M+H]⁺: 415.0. Found 415.1.

### Step 4: 5-((3-Iodo-1H-indazol-5-yl)amino)-5,6,7,8-tetrahydronaphthalene-2-carbonitrile, enantiomer 1 and 2

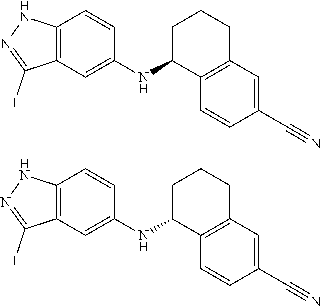 [SMILES: N#Cc1ccc2c(c1)CCC[C@@H]2Nc1ccc2[nH]nc(I)c2c1 ; N#Cc1ccc2c(c1)CCC[C@H]2Nc1ccc2[nH]nc(I)c2c1]

5-((3-Iodo-1H-indazol-5-yl)amino)-5,6,7,8-tetrahydronaphthalene-2-carbonitrile was subjected to chiral separation using Method CH to afford 5-((3-iodo-1H-indazol-5-yl)amino)-5,6,7,8-tetrahydronaphthalene-2-carbonitrile, enantiomer 1 (640 mg, 40%) as a yellow solid. ¹H NMR (400 MHz, DMSO-d₆) δ 13.09 (br s, 1H), 7.63 (s, 1H), 7.59-7.56 (m, 1H), 7.52-7.47 (m, 1H), 7.31 (d, J=8.9 Hz, 1H), 7.00 (dd, J=1.9, 9.0 Hz, 1H), 6.42 (s, 1H), 5.97 (d, J=8.9 Hz, 1H), 4.68 (br d, J=7.7 Hz, 1H), 2.88-2.73 (m, 2H), 2.00-1.77 (m, 4H). MS-ESI (m/z) calc'd for C₁₈H₁₆IN₄ [M+H]⁺: 415.0. Found 415.0. A later eluting fraction was also isolated to afford 5-((3-iodo-1H-indazol-5-yl)amino)-5,6,7,8-tetrahydronaphthalene-2-carbonitrile, enantiomer 2 (540 mg, 39%) as a yellow solid. ¹H NMR (400 MHz, DMSO-d₆) δ 13.09 (s, 1H), 7.63 (s, 1H), 7.60-7.55 (m, 1H), 7.52-7.48 (m, 1H), 7.31 (d, J=8.9 Hz, 1H), 7.00 (dd, J=2.0, 9.0 Hz, 1H), 6.42 (s, 1H), 5.97 (d, J=8.8 Hz, 1H), 4.68 (br d, J=7.0 Hz, 1H), 2.88-2.71 (m, 2H), 2.01-1.77 (m, 4H). MS-ESI (m/z) calc'd for C₁₈H₁₆IN₄ [M+H]⁺: 415.0. Found 415.0.

### Step 5: 5-((3-(Cyclohex-1-en-1-yl)-1H-indazol-5-yl)amino)-5,6,7,8-tetrahydronaphthalene-2-carbonitrile, enantiomer 1

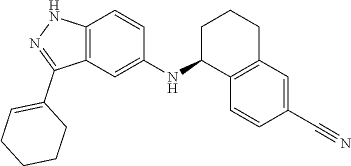 [SMILES: N#Cc1ccc2c(c1)CCC[C@@H]2Nc1ccc2[nH]nc(C3=CCCCC3)c2c1]

A mixture of 5-((3-iodo-1H-indazol-5-yl)amino)-5,6,7,8-tetrahydronaphthalene-2-carbonitrile, enantiomer 1 (50 mg, 120.70 umol), 1-cyclohexen-1-yl-boronic acid (19.76 mg, 156.91 umol), bis(4-(di-tert-butylphosphanyl)-N,N-dimethylaniline); dichloropalladium (8.55 mg, 12.07 umol) and AcOK (35.54 mg, 362.11 umol) in EtOH (2 mL) and H₂O (0.5 mL) was degassed and purged with N₂ (3×) at 20° C., and then the mixture was stirred at 100° C. for 12 hrs under an N₂ atmosphere. The reaction mixture was concentrated to give a residue. The residue was purified by preparative-HPLC using Method BI. The material was further purified by preparative-HPLC using Method CJ to afford the title compound (12.31 mg, 20%) as a white solid. ¹H NMR (400 MHz, DMSO-d₆) δ 12.44 (s, 1H), 7.62 (s, 1H), 7.59-7.49 (m, 2H), 7.26 (d, J=8.8 Hz, 1H), 6.99 (s, 1H), 6.91 (dd, J=1.8, 9.0 Hz, 1H), 6.32 (br s, 1H), 5.71 (d, J=9.0 Hz, 1H), 4.75-4.66 (m, 1H), 2.86-2.74 (m, 2H), 2.53 (br d, J=4.0 Hz, 2H), 2.22 (br d, J=3.1 Hz, 2H), 1.97-1.77 (m, 4H), 1.75-1.68 (m, 2H), 1.68-1.60 (m, 2H). MS-ESI (m/z) calc'd for C₂₄H₂₅N₄ [M+H]⁺: 369.2. Found 369.2.

### Example 55: 4-(5-((6-Cyano-1,2,3,4-tetrahydronaphthalen-1-yl)amino)-1H-indazol-3-yl)benzoic acid, enantiomer 1

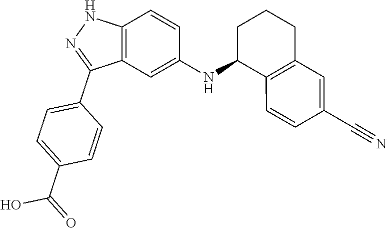 [SMILES: N#Cc1ccc2c(c1)CCC[C@@H]2Nc1ccc2[nH]nc(-c3ccc(C(=O)O)cc3)c2c1]

Prepared as described for 5-((3-(cyclohex-1-en-1-yl)-1H-indazol-5-yl)amino)-5,6,7,8-tetrahydronaphthalene-2-carbonitrile, enantiomer 1, using 4-boronobenzoic acid in place of cyclohex-1-en-1-ylboronic acid to afford the title compound (1.96 mg, 9%) as a yellow gum. ¹H NMR (400 MHz, DMSO-d₆) δ 13.05 (br s, 1H), 8.01 (s, 4H), 7.64 (s, 1H), 7.59-7.51 (m, 2H), 7.38 (d, J=8.9 Hz, 1H), 7.16 (s, 1H), 7.00 (d, J=8.9 Hz, 1H), 5.92 (br d, J=8.4 Hz, 1H), 4.81 (br d, J=7.5 Hz, 1H), 4.09 (br s, 1H), 2.82 (br d, J=6.0 Hz, 2H), 2.01-1.76 (m, 4H). MS-ESI (m/z) calc'd for C₂₅H₂₁N₄O₂ [M+H]⁺: 409.2. Found 409.1.

### Example 56: 5-((2′-Methyl-1H,2′H-[3,6′-biindazol]-5-yl)amino)-5,6,7,8-tetrahydronaphthalene-2-carbonitrile, enantiomer 1

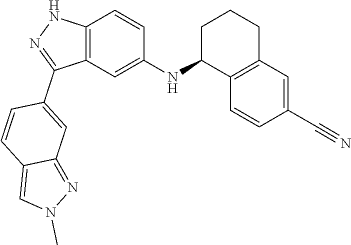 [SMILES: Cn1cc2ccc(-c3n[nH]c4ccc(N[C@H]5CCCc6cc(C#N)ccc65)cc34)cc2n1]

Prepared as described for 5-((3-(cyclohex-1-en-1-yl)-1H-indazol-5-yl)amino)-5,6,7,8-tetrahydronaphthalene-2-carbonitrile, enantiomer 1, using 2-methyl-6-(4,4,5,5-tetramethyl-1,3,2-dioxaborolan-2-yl)indazole in place of cyclohex-1-en-1-ylboronic acid to afford the title compound (6.82 mg, 25%) as a yellow solid. ¹H NMR (400 MHz, DMSO-d₆) δ 8.35 (s, 1H), 8.03 (s, 1H), 7.84-7.73 (m, 1H), 7.70-7.65 (m, 2H), 7.63-7.57 (m, 2H), 7.48 (br d, J=8.8 Hz, 2H), 7.13 (br d, J=8.4 Hz, 1H), 4.87 (br s, 1H), 4.19 (s, 3H), 2.94-2.75 (m, 2H), 1.99-1.74 (m, 4H). MS-ESI (m/z) calc'd for C₂₆H₂₃N₆ [M+H]⁺: 419.2. Found 419.1.

### Example 57: 5-((3-(3-(Methylsulfonyl)phenyl)-1H-indazol-5-yl)amino)-5,6,7,8-tetrahydronaphthalene-2-carbonitrile, enantiomer 1

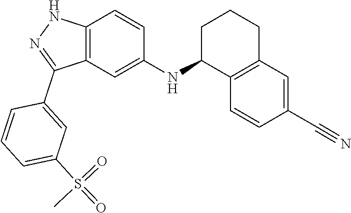 [SMILES: CS(=O)(=O)c1cccc(-c2n[nH]c3ccc(N[C@H]4CCCc5cc(C#N)ccc54)cc23)c1]

Prepared as described for 5-((3-(cyclohex-1-en-1-yl)-1H-indazol-5-yl)amino)-5,6,7,8-tetrahydronaphthalene-2-carbonitrile, enantiomer 1, using (3-methylsulfonylphenyl)boronic acid in place of cyclohex-1-en-1-ylboronic acid to afford the title compound (9.66 mg, 35%) as a pink solid. ¹H NMR (400 MHz, DMSO-d₆) δ 8.41 (s, 1H), 8.23 (d, J=7.7 Hz, 1H), 7.89 (d, J=7.9 Hz, 1H), 7.79-7.74 (m, 1H), 7.66 (s, 1H), 7.62-7.54 (m, 2H), 7.46 (d, J=8.8 Hz, 1H), 7.25 (br s, 1H), 7.09 (br d, J=9.0 Hz, 1H), 4.82 (br t, J=4.4 Hz, 1H), 3.27 (s, 3H), 2.91-2.74 (m, 2H), 2.01-1.73 (m, 4H). MS-ESI (m/z) calc'd for C₂₅H₂₃N₄₀₂S [M+H]⁺: 443.2. Found 443.0.

### Example 58: 5-((3-(3,5-Dimethoxyphenyl)-1H-indazol-5-yl)amino)-5,6,7,8-tetrahydronaphthalene-2-carbonitrile, enantiomer 1

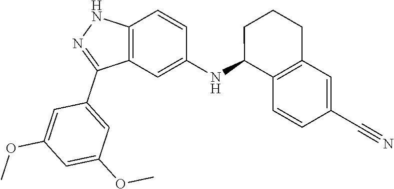 [SMILES: COc1cc(OC)cc(-c2n[nH]c3ccc(N[C@H]4CCCc5cc(C#N)ccc54)cc23)c1]

Prepared as described for 5-((3-(cyclohex-1-en-1-yl)-1H-indazol-5-yl)amino)-5,6,7,8-tetrahydronaphthalene-2-carbonitrile, enantiomer 1, using 2-(3,5-dimethoxyphenyl)-4,4,5,5-tetramethyl-1,3,2-dioxaborolane in place of cyclohex-1-en-1-ylboronic acid to afford the title compound (6.07 mg, 23%) as a pink solid. ¹H NMR (400 MHz, DMSO-d₆) δ 7.63 (s, 1H), 7.60-7.56 (m, 1H), 7.55-7.52 (m, 1H), 7.41 (br d, J=8.8 Hz, 1H), 7.23-7.12 (m, 1H), 7.06 (br d, J=8.4 Hz, 1H), 6.97 (d, J=2.2 Hz, 2H), 6.47 (t, J=2.2 Hz, 1H), 4.73 (br s, 1H), 3.77 (s, 6H), 2.88-2.72 (m, 2H), 2.04-1.67 (m, 4H). MS-ESI (m/z) calc'd for C₂₆H₂₅N₄O₂ [M+H]⁺: 425.2. Found 425.1.

### Example 59: 5-((3-(Benzo[d]thiazol-6-yl)-1H-indazol-5-yl)amino)-5,6,7,8-tetrahydronaphthalene-2-carbonitrile

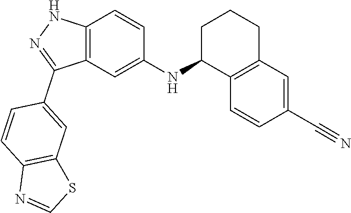 [SMILES: N#Cc1ccc2c(c1)CCC[C@@H]2Nc1ccc2[nH]nc(-c3ccc4ncsc4c3)c2c1]

Prepared as described for 5-((3-(cyclohex-1-en-1-yl)-1H-indazol-5-yl)amino)-5,6,7,8-tetrahydronaphthalene-2-carbonitrile, enantiomer 1, using 6-(4,4,5,5-tetramethyl-1,3,2-dioxaborolan-2-yl)-1,3-benzothiazole in place of cyclohex-1-en-1-ylboronic acid to afford the title compound (7.6 mg, 37%,) as a yellow solid. ¹H NMR (400 MHz, MeOH-d₄) δ 9.14 (s, 1H) 8.37 (d, J=1.00 Hz, 1H) 8.03-8.09 (m, 1H) 7.92-8.00 (m, 1H) 7.52 (d, J=8.00 Hz, 1H) 7.41 (s, 1H) 7.26-7.36 (m, 2H) 7.03 (s, 1H) 6.94 (dd, J=8.94, 1.94 Hz, 1H) 4.64 (br t, J=5.88 Hz, 1H) 2.66-2.85 (m, 2H) 1.72-2.00 (m, 4H). MS-ESI (m/z) calc'd for C₂₅H₂₀N₅S [M+H]⁺: 422.1. Found 422.1.

### Example 60: 5-((3-(2-Methyloxazol-5-yl)-1H-indazol-5-yl)amino)-5,6,7,8-tetrahydronaphthalene-2-carbonitrile, enantiomer 1

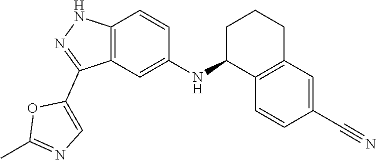 [SMILES: Cc1ncc(-c2n[nH]c3ccc(N[C@H]4CCCc5cc(C#N)ccc54)cc23)o1]

Prepared as described for 5-((3-(cyclohex-1-en-1-yl)-1H-indazol-5-yl)amino)-5,6,7,8-tetrahydronaphthalene-2-carbonitrile, enantiomer 1, using 2-methyl-5-(4,4,5,5-tetramethyl-1,3,2-dioxaborolan-2-yl)oxazole in place of cyclohex-1-en-1-ylboronic acid to afford the title compound (15.56 mg, 82%) as a pale yellow solid. ¹H NMR (400 MHz, DMSO-d₆) δ 13.00 (s, 1H), 7.64 (s, 1H), 7.59-7.55 (m, 1H), 7.55-7.51 (m, 1H), 7.49 (s, 1H), 7.35 (d, J=9.6 Hz, 1H), 7.04-6.95 (m, 2H), 5.94 (d, J=9.1 Hz, 1H), 4.83 (br d, J=8.5 Hz, 1H), 2.91-2.75 (m, 2H), 2.01-1.78 (m, 4H). MS-ESI (m/z) calc'd for C₂₂H₂₀N₅O [M+H]⁺: 370.2. Found 370.0.

### Example 61: 5-((3-(1H-Indol-6-yl)-1H-indazol-5-yl)amino)-5,6,7,8-tetrahydronaphthalene-2-carbonitrile, enantiomer 1

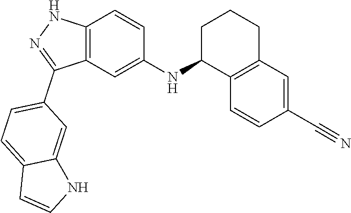 [SMILES: N#Cc1ccc2c(c1)CCC[C@@H]2Nc1ccc2[nH]nc(-c3ccc4cc[nH]c4c3)c2c1]

Prepared as described for 5-((3-(cyclohex-1-en-1-yl)-1H-indazol-5-yl)amino)-5,6,7,8-tetrahydronaphthalene-2-carbonitrile, enantiomer 1, using 6-(4,4,5,5-tetramethyl-1,3,2-dioxaborolan-2-yl)-1H-indole in place of cyclohex-1-en-1-ylboronic acid to afford the title compound (3.34 mg, 16%) as a yellow solid. ¹H NMR (400 MHz, DMSO-d₆) δ 12.65 (s, 1H), 11.07 (br s, 1H), 7.86 (s, 1H), 7.61 (s, 1H), 7.59-7.55 (m, 1H), 7.54 (s, 2H), 7.52-7.47 (m, 1H), 7.38-7.24 (m, 2H), 7.12 (s, 1H), 6.95 (dd, J=1.8, 8.8 Hz, 1H), 6.42 (br s, 1H), 5.80 (d, J=9.0 Hz, 1H), 4.80-4.59 (m, 1H), 2.87-2.71 (m, 2H), 2.00-1.71 (m, 4H). MS-ESI (m/z) calc'd for C₂₆H₂₂N₅ [M+H]⁺: 404.2. Found 404.1.

### Example 62: 5-((3-(1-Methyl-6-oxo-1,6-dihydropyridin-3-yl)-1H-indazol-5-yl)amino)-5,6,7,8-tetrahydronaphthalene-2-carbonitrile, enantiomer 1

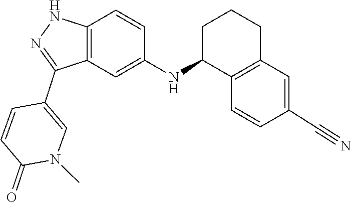 [SMILES: Cn1cc(-c2n[nH]c3ccc(N[C@H]4CCCc5cc(C#N)ccc54)cc23)ccc1=O]

Prepared as described for 5-((3-(cyclohex-1-en-1-yl)-1H-indazol-5-yl)amino)-5,6,7,8-tetrahydronaphthalene-2-carbonitrile, enantiomer 1, using 1-methyl-5-(4,4,5,5-tetramethyl-1,3,2-dioxaborolan-2-yl)pyridin-2-one in place of cyclohex-1-en-1-ylboronic acid to afford the title compound (7.89 mg, 41%) as a yellow solid. ¹H NMR (400 MHz, MeOD) δ 7.89-8.02 (m, 2H) 7.50 (d, J=8.00 Hz, 1H) 7.40 (s, 1H) 7.35 (d, J=8.00 Hz, 1H) 7.27 (d, J=9.63 Hz, 1H) 6.88-6.94 (m, 2H) 6.57 (d, J=9.63 Hz, 1H) 4.66 (br t, J=5.44 Hz, 1H) 3.56 (s, 3H) 2.67-2.86 (m, 2H) 1.72-2.02 (m, 4H). MS-ESI (m/z) calc'd for C₂₄H₂₂N₅O [M+H]⁺: 396.2. Found 396.0.

### Example 63: 5-((3-(4-Cyanophenyl)-1H-indazol-5-yl)amino)-5,6,7,8-tetrahydronaphthalene-2-carbonitrile, enantiomer 1

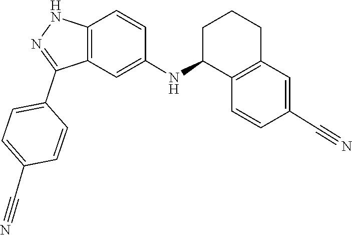 [SMILES: N#Cc1ccc(-c2n[nH]c3ccc(N[C@H]4CCCc5cc(C#N)ccc54)cc23)cc1]

Prepared as described for 5-((3-(cyclohex-1-en-1-yl)-1H-indazol-5-yl)amino)-5,6,7,8-tetrahydronaphthalene-2-carbonitrile, enantiomer 1, using (4-cyanophenyl)boronic acid in place of cyclohex-1-en-1-ylboronic acid to afford the title compound (9.42 mg, 45%) as a green solid. ¹H NMR (400 MHz, MeOD) δ 8.06 (d, J=8.4 Hz, 2H), 7.81 (d, J=8.6 Hz, 2H), 7.61 (d, J=8.2 Hz, 1H), 7.52 (s, 1H), 7.46 (d, J=9.5 Hz, 1H), 7.41 (d, J=9.0 Hz, 1H), 7.12 (s, 1H), 7.03 (dd, J=2.0, 9.0 Hz, 1H), 4.79-4.73 (m, 1H), 2.98-2.79 (m, 2H), 2.12-1.74 (m, 4H). MS-ESI (m/z) calc'd for C₂₅H₂₀N₅ [M+H]⁺: 390.2. Found 390.1.

### Example 64: 5-((3-(Pyridin-4-yl)-1H-indazol-5-yl)amino)-5,6,7,8-tetrahydronaphthalene-2-carbonitrile, enantiomer 1

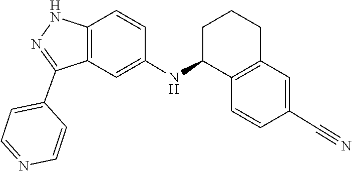 [SMILES: N#Cc1ccc2c(c1)CCC[C@@H]2Nc1ccc2[nH]nc(-c3ccncc3)c2c1]

Prepared as described for 5-((3-(cyclohex-1-en-1-yl)-1H-indazol-5-yl)amino)-5,6,7,8-tetrahydronaphthalene-2-carbonitrile, enantiomer 1, using 4-pyridylboronic acid in place of cyclohex-1-en-1-ylboronic acid to afford the title compound (12.61 mg, 20%) as a yellow solid. ¹H NMR (400 MHz, DMSO-d₆) δ 13.21 (s, 1H), 8.67-8.56 (m, 2H), 7.89 (d, J=6.0 Hz, 2H), 7.65 (s, 1H), 7.59-7.52 (m, 2H), 7.41 (d, J=8.8 Hz, 1H), 7.19 (s, 1H), 7.01 (dd, J=1.9, 8.9 Hz, 1H), 5.97 (d, J=9.0 Hz, 1H), 4.90-4.80 (m, 1H), 2.89-2.75 (m, 2H), 2.01-1.78 (m, 4H). MS-ESI (m/z) calc'd for C₂₃H₂₀N₅ [M+H]⁺: 366.2. Found 366.2.

### Example 65: 5-((3-(Cyclopent-1-en-1-yl)-1H-indazol-5-yl)amino)-5,6,7,8-tetrahydronaphthalene-2-carbonitrile, enantiomer 1

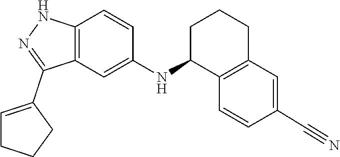 [SMILES: N#Cc1ccc2c(c1)CCC[C@@H]2Nc1ccc2[nH]nc(C3=CCCC3)c2c1]

Prepared as described for 5-((3-(cyclohex-1-en-1-yl)-1H-indazol-5-yl)amino)-5,6,7,8-tetrahydronaphthalene-2-carbonitrile, enantiomer 1, using cyclopenten-1-ylboronic acid in place of cyclohex-1-en-1-ylboronic acid to afford the title compound (3.35 mg, 14%) as a green solid. ¹H NMR (400 MHz, DMSO-d₆) δ 12.52 (s, 1H), 7.62 (s, 1H), 7.59-7.49 (m, 2H), 7.28 (d, J=8.8 Hz, 1H), 7.02 (s, 1H), 6.93 (dd, J=1.9, 8.9 Hz, 1H), 6.30 (br s, 1H), 5.75 (d, J=9.3 Hz, 1H), 4.76 (br d, J=8.2 Hz, 1H), 2.81 (br s, 4H), 2.54 (br s, 2H), 1.98-1.86 (m, 4H), 1.83 (br d, J=10.8 Hz, 2H). MS-ESI (m/z) calc'd for C₂₃H₂₃N₄ [M+H]⁺: 355.2. Found 355.1.

### Example 66: 5-(5-((6-Cyano-1,2,3,4-tetrahydronaphthalen-1-yl)amino)-1H-indazol-3-yl)-2-fluorobenzoic acid, enantiomer 1

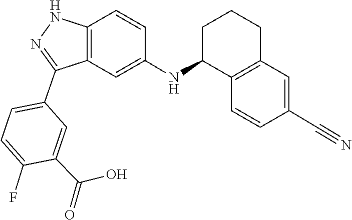 [SMILES: N#Cc1ccc2c(c1)CCC[C@@H]2Nc1ccc2[nH]nc(-c3ccc(F)c(C(=O)O)c3)c2c1]

Prepared as described for 5-((3-(cyclohex-1-en-1-yl)-1H-indazol-5-yl)amino)-5,6,7,8-tetrahydronaphthalene-2-carbonitrile, enantiomer 1, using 5-borono-2-fluoro-benzoic acid in place of cyclohex-1-en-1-ylboronic acid to afford the title compound (10.84 mg, 52%) as a pale yellow solid. ¹H NMR (400 MHz, DMSO-d₆) δ 12.88 (br s, 1H), 8.23 (br d, J=5.07 Hz, 1H), 7.91 (br s, 1H), 7.62 (s, 1H), 7.55 (s, 2H), 7.36 (d, J=9.04 Hz, 1H), 7.26 (br t, J=9.70 Hz, 1H), 7.07 (s, 1H), 6.99 (br d, J=8.82 Hz, 1H), 5.89 (br d, J=8.38 Hz, 1H), 4.72 (br s, 1H), 2.74-2.89 (m, 2H), 1.75-2.01 (m, 4H). MS-ESI (m/z) calc'd for C₂₅H₂₀FN₄O₂ [M+H]⁺: 427.2. Found 427.1.

### Example 67: 5-((3-(Benzo[d]thiazol-5-yl)-1H-indazol-5-yl)amino)-5,6,7,8-tetrahydronaphthalene-2-carbonitrile, enantiomer 1

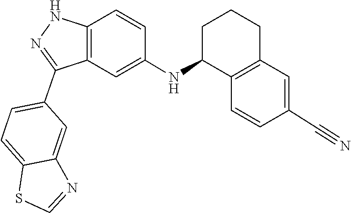 [SMILES: N#Cc1ccc2c(c1)CCC[C@@H]2Nc1ccc2[nH]nc(-c3ccc4scnc4c3)c2c1]

Prepared as described for 5-((3-(cyclohex-1-en-1-yl)-1H-indazol-5-yl)amino)-5,6,7,8-tetrahydronaphthalene-2-carbonitrile, enantiomer 1, using 5-(4,4,5,5-tetramethyl-1,3,2-dioxaborolan-2-yl)benzo[d]thiazole in place of cyclohex-1-en-1-ylboronic acid to afford the title compound (4.28 mg, 20%) as a white solid. ¹H NMR (400 MHz, DMSO-d₆) δ 12.98 (s, 1H) 9.44 (s, 1H) 8.51 (s, 1H) 8.25 (d, J=8 Hz, 1H) 8.06 (d, J=8 Hz, 1H) 7.65 (s, 1H) 7.54-7.60 (m, 2H) 7.40 (d, J=9 Hz, 1H) 7.21 (s, 1H) 7.03 (dd, J=9, 2 Hz, 1H) 5.94 (d, J=9 Hz, 1H) 4.75-4.83 (m, 1H) 2.74-2.89 (m, 2H) 1.74-2.02 (m, 4H). MS-ESI (m/z) calc'd for C₂₅H₂₀N₅S [M+H]⁺: 422.1. Found 422.0.

### Example 68: 5-((3-(3-(Difluoromethyl)phenyl)-1H-indazol-5-yl)amino)-5,6,7,8-tetrahydronaphthalene-2-carbonitrile, enantiomer 1

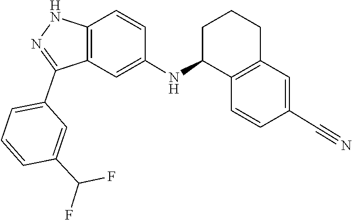 [SMILES: N#Cc1ccc2c(c1)CCC[C@@H]2Nc1ccc2[nH]nc(-c3cccc(C(F)F)c3)c2c1]

Prepared as described for 5-((3-(cyclohex-1-en-1-yl)-1H-indazol-5-yl)amino)-5,6,7,8-tetrahydronaphthalene-2-carbonitrile, enantiomer 1, using (3-(difluoromethyl)phenyl)boronic acid in place of cyclohex-1-en-1-ylboronic acid to afford the title compound (4.65 mg, 22%) as a white solid. ¹H NMR (400 MHz, DMSO-d₆) δ 13.01 (s, 1H) 8.03-8.09 (m, 2H) 7.49-7.68 (m, 5H) 7.39 (d, J=9 Hz, 1H) 6.97-7.28 (m, 3H) 5.96 (d, J=9 Hz, 1H) 4.74-4.82 (m, 1H) 2.74-2.90 (m, 2H) 1.74-1.99 (m, 4H). MS-ESI (m/z) calc'd for C₂₅H₂₁F₂N₄ [M+H]⁺: 415.2. Found 415.1.

### Example 69: 3-(5-((6-Cyano-1,2,3,4-tetrahydronaphthalen-1-yl)amino)-1H-indazol-3-yl)-N-methoxybenzamide, enantiomer 1

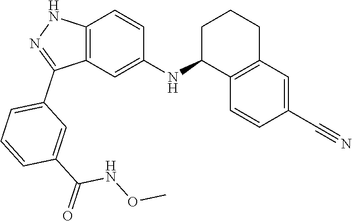 [SMILES: CONC(=O)c1cccc(-c2n[nH]c3ccc(N[C@H]4CCCc5cc(C#N)ccc54)cc23)c1]

Prepared as described for 5-((3-(cyclohex-1-en-1-yl)-1H-indazol-5-yl)amino)-5,6,7,8-tetrahydronaphthalene-2-carbonitrile, enantiomer 1, using (3-(methoxycarbamoyl)phenyl)boronic acid in place of cyclohex-1-en-1-ylboronic acid to afford the title compound (4.28 mg, 20%) as a green solid. ¹H NMR (400 MHz, DMSO-d₆) δ 12.97 (s, 1H) 11.83 (br s, 1H) 8.28 (s, 1H) 8.05 (br d, J=8 Hz, 1H) 7.69 (d, J=8 Hz, 1H) 7.64 (s, 1H) 7.53-7.59 (m, 3H) 7.39 (d, J=9 Hz, 1H) 7.14 (s, 1H) 7.01 (dd, J=9, 2 Hz, 1H) 5.92 (d, J=9 Hz, 1H) 4.73-4.82 (m, 1H) 3.72 (s, 3H) 2.74-2.91 (m, 2H) 1.73-2.02 (m, 4H). MS-ESI (m/z) calc'd for C₂₆H₂₄N₅O₂ [M+H]⁺: 438.2. Found 438.2.

### Example 70: 5-((3-(4-Nitrophenyl)-1H-indazol-5-yl)amino)-5,6,7,8-tetrahydronaphthalene-2-carbonitrile, enantiomer 1

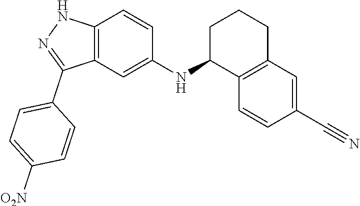 [SMILES: N#Cc1ccc2c(c1)CCC[C@@H]2Nc1ccc2[nH]nc(-c3ccc([N+](=O)[O-])cc3)c2c1]

Prepared as described for 5-((3-(cyclohex-1-en-1-yl)-1H-indazol-5-yl)amino)-5,6,7,8-tetrahydronaphthalene-2-carbonitrile, enantiomer 1, using (4-nitrophenyl)boronic acid in place of cyclohex-1-en-1-ylboronic acid to afford the title compound (8.38 mg, 42%) as a yellow solid. ¹H NMR (400 MHz, DMSO-d₆) δ 13.29 (br s, 1H), 8.28-8.34 (m, 2H), 8.20 (d, J=8.82 Hz, 2H), 7.65 (s, 1H), 7.53-7.59 (m, 2H), 7.42 (d, J=8.82 Hz, 1H), 7.20 (s, 1H), 7.02 (d, J=9.26 Hz, 1H), 6.01 (d, J=9.04 Hz, 1H), 4.87 (br s, 1H), 2.78-2.88 (m, 2H), 1.80-2.04 (m, 4H). MS-ESI (m/z) calc'd for C₂₄H₂₀N₅O₂ [M+H]⁺: 410.2. Found 410.1.

### Example 71: 5-((3-(3,4-Dimethoxyphenyl)-1H-indazol-5-yl)amino)-5,6,7,8-tetrahydronaphthalene-2-carbonitrile, enantiomer 1

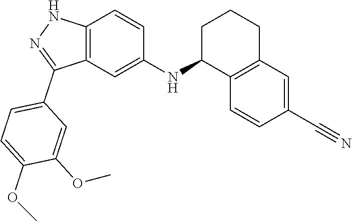 [SMILES: COc1ccc(-c2n[nH]c3ccc(N[C@H]4CCCc5cc(C#N)ccc54)cc23)cc1OC]

Prepared as described for 5-((3-(cyclohex-1-en-1-yl)-1H-indazol-5-yl)amino)-5,6,7,8-tetrahydronaphthalene-2-carbonitrile, enantiomer 1, using (3,4-dimethoxyphenyl)boronic acid in place of cyclohex-1-en-1-ylboronic acid to afford the title compound (1.76 mg, 7%) as a yellow solid. ¹H NMR (400 MHz, DMSO-d₆) δ 12.70 (s, 1H), 7.63 (s, 1H), 7.53-7.60 (m, 2H), 7.32-7.44 (m, 3H), 6.93-7.13 (m, 3H), 5.83 (d, J=8.82 Hz, 1H), 4.71 (br d, J=8.16 Hz, 1H), 3.79 (d, J=1.32 Hz, 6H), 2.72-2.91 (m, 2H), 1.76-2.00 (m, 4H). MS-ESI (m/z) calc'd for C₂₆H₂₅N₄O₂ [M+H]⁺: 425.2. Found 425.1.

### Example 72: 5-((3-(4-Morpholinophenyl)-1H-indazol-5-yl)amino)-5,6,7,8-tetrahydronaphthalene-2-carbonitrile, enantiomer 1

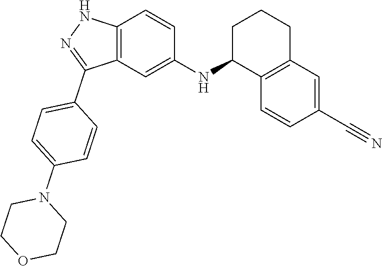 [SMILES: N#Cc1ccc2c(c1)CCC[C@@H]2Nc1ccc2[nH]nc(-c3ccc(N4CCOCC4)cc3)c2c1]

Prepared as described for 5-((3-(cyclohex-1-en-1-yl)-1H-indazol-5-yl)amino)-5,6,7,8-tetrahydronaphthalene-2-carbonitrile, enantiomer 1, using (4-morpholinophenyl)boronic acid in place of cyclohex-1-en-1-ylboronic acid to afford the title compound (6.32 mg, 29%) as a yellow solid. ¹H NMR (400 MHz, DMSO-d₆) δ 12.63 (s, 1H), 7.74 (d, J=8.82 Hz, 2H), 7.64 (s, 1H), 7.52-7.59 (m, 2H), 7.32 (d, J=8.82 Hz, 1H), 7.00-7.09 (m, 3H), 6.96 (br d, J=9.26 Hz, 1H), 5.79 (d, J=9.26 Hz, 1H), 4.72 (br s, 1H), 3.72-3.80 (m, 4H), 3.11-3.19 (m, 4H), 2.74-2.88 (m, 2H), 1.74-2.01 (m, 4H). MS-ESI (m/z) calc'd for C₂₈H₂₈N₅O [M+H]⁺: 450.2. Found 450.1.

### Example 73: 5-((3-(4-Methoxy-3-methylphenyl)-1H-indazol-5-yl)amino)-5,6,7,8-tetrahydronaphthalene-2-carbonitrile, enantiomer 1

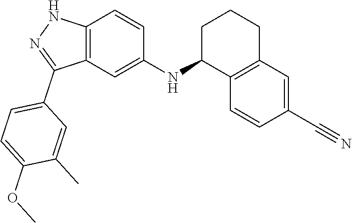 [SMILES: COc1ccc(-c2n[nH]c3ccc(N[C@H]4CCCc5cc(C#N)ccc54)cc23)cc1C]

Prepared as described for 5-((3-(cyclohex-1-en-1-yl)-1H-indazol-5-yl)amino)-5,6,7,8-tetrahydronaphthalene-2-carbonitrile, enantiomer 1, using (4-methoxy-3-methyl-phenyl)boronic acid in place of cyclohex-1-en-1-ylboronic acid to afford the title compound (10.39 mg, 50%) as a yellow solid. ¹H NMR (400 MHz, DMSO-d₆) δ 12.65 (br s, 1H), 7.59-7.66 (m, 3H), 7.51-7.58 (m, 2H), 7.31 (d, J=8.82 Hz, 1H), 6.98 (td, J=8.21, 16.21 Hz, 3H), 5.79 (d, J=8.82 Hz, 1H), 4.65-4.76 (m, 1H), 3.81 (s, 3H), 2.72-2.88 (m, 2H), 2.20 (s, 3H), 1.73-1.99 (m, 4H). MS-ESI (m/z) calc'd for C₂₆H₂₅N₄O [M+H]⁺:409.2. Found 409.1.

### Example 74: 5-((3-(4-(Methylsulfonyl)phenyl)-1H-indazol-5-yl)amino)-5,6,7,8-tetrahydronaphthalene-2-carbonitrile, enantiomer 1

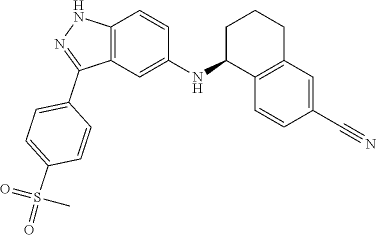 [SMILES: CS(=O)(=O)c1ccc(-c2n[nH]c3ccc(N[C@H]4CCCc5cc(C#N)ccc54)cc23)cc1]

Prepared as described for 5-((3-(cyclohex-1-en-1-yl)-1H-indazol-5-yl)amino)-5,6,7,8-tetrahydronaphthalene-2-carbonitrile, enantiomer 1, using (4-methylsulfonylphenyl)boronic acid in place of cyclohex-1-en-1-ylboronic acid to afford the title compound (4.21 mg, 15%) as a yellow solid. ¹H NMR (400 MHz, DMSO-d₆) δ 13.25 (s, 1H), 8.23 (d, J=8.60 Hz, 2H), 8.05 (d, J=8.60 Hz, 2H), 7.71 (s, 1H), 7.62 (q, J=8.09 Hz, 2H), 7.49 (d, J=9.04 Hz, 1H), 7.31 (br s, 1H), 7.10 (br d, J=8.82 Hz, 1H), 4.91 (br s, 1H), 3.30 (s, 3H), 2.79-2.96 (m, 2H), 1.79-2.07 (m, 4H). MS-ESI (m/z) calc'd for C₂₅H₂₃N₄₀₂S [M+H]⁺: 443.2. Found 443.2.

### Example 75: (E)-5-((3-(Prop-1-en-1-yl)-1H-indazol-5-yl)amino)-5,6,7,8-tetrahydronaphthalene-2-carbonitrile, enantiomer 1

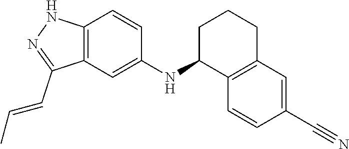 [SMILES: C/C=C/c1n[nH]c2ccc(N[C@H]3CCCc4cc(C#N)ccc43)cc12]

Prepared as described for 5-((3-(cyclohex-1-en-1-yl)-1H-indazol-5-yl)amino)-5,6,7,8-tetrahydronaphthalene-2-carbonitrile, enantiomer 1, using 4,4,5,5-tetramethyl-2-[(E)-prop-1-enyl]-1,3,2-dioxaborolane in place of cyclohex-1-en-1-ylboronic acid to afford the title compound (2.43 mg, 11%) as a yellow solid. ¹H NMR (400 MHz, DMSO-d₆) δ 12.49 (s, 1H), 7.63 (s, 1H), 7.51-7.60 (m, 2H), 7.26 (d, J=8.80 Hz, 1H), 7.00 (s, 1H), 6.92 (dd, J=8.93, 1.59 Hz, 1H), 6.64 (dd, J=16.14, 1.59 Hz, 1H), 6.31-6.43 (m, 1H), 5.75 (br d, J=9.05 Hz, 1H), 4.69-4.81 (m, 1H), 2.73-2.92 (m, 2H), 1.79-2.00 (m, 7H). MS-ESI (m/z) calc'd for C₂₁H₂₁N₄ [M+H]⁺: 329.2. Found 329.1.

### Example 76: 5-((3-(5-Cyclopropylpyridin-3-yl)-1H-indazol-5-yl)amino)-5,6,7,8-tetrahydronaphthalene-2-carbonitrile, enantiomer 1

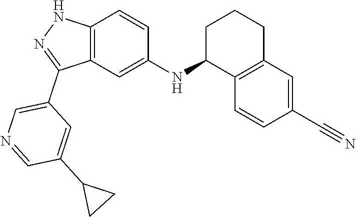 [SMILES: N#Cc1ccc2c(c1)CCC[C@@H]2Nc1ccc2[nH]nc(-c3cncc(C4CC4)c3)c2c1]

Prepared as described for 5-((3-(cyclohex-1-en-1-yl)-1H-indazol-5-yl)amino)-5,6,7,8-tetrahydronaphthalene-2-carbonitrile, enantiomer 1, using (5-cyclopropylpyridin-3-yl)boronic acid in place of cyclohex-1-en-1-ylboronic acid to afford the title compound (2.02 mg, 10%) as a yellow solid. ¹H NMR (400 MHz, DMSO-d₆) δ 13.02 (s, 1H), 8.86 (d, J=1.59 Hz, 1H), 8.36 (d, J=1.96 Hz, 1H), 7.77 (s, 1H), 7.64 (s, 1H), 7.51-7.61 (m, 2H), 7.39 (d, J=8.80 Hz, 1H), 6.98-7.08 (m, 2H), 5.94 (d, J=8.68 Hz, 1H), 4.76 (br d, J=8.19 Hz, 1H), 2.74-2.91 (m, 2H), 1.94-2.09 (m, 2H), 1.76-1.91 (m, 3H), 1.00-1.10 (m, 2H), 0.73-0.85 (m, 2H). MS-ESI (m/z) calc'd for C₂₆H₂₄N₅ [M+H]⁺: 406.2. Found 406.1.

### Example 77: 5-((3-(Benzo[d][1,3]dioxol-5-yl)-1H-indazol-5-yl)amino)-5,6,7,8-tetrahydronaphthalene-2-carbonitrile, enantiomer 1

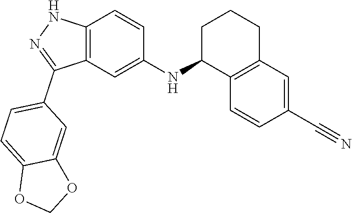 [SMILES: N#Cc1ccc2c(c1)CCC[C@@H]2Nc1ccc2[nH]nc(-c3ccc4c(c3)OCO4)c2c1]

Prepared as described for 5-((3-(cyclohex-1-en-1-yl)-1H-indazol-5-yl)amino)-5,6,7,8-tetrahydronaphthalene-2-carbonitrile, enantiomer 1, using benzo[d][1,3]dioxol-5-ylboronic acid in place of cyclohex-1-en-1-ylboronic acid to afford the title compound (4.03 mg, 16%) as a white solid. ¹H NMR (400 MHz, DMSO-d₆) δ 12.75 (br s, 1H), 7.64 (s, 1H), 7.50-7.60 (m, 2H), 7.30-7.40 (m, 3H), 6.93-7.08 (m, 3H), 6.06 (s, 2H), 5.84 (br d, J=9.05 Hz, 1H), 4.76 (br d, J=7.58 Hz, 1H), 2.74-2.91 (m, 2H), 1.77-2.01 (m, 4H). MS-ESI (m/z) calc'd for C₂₅H₂₁N₄O₂ [M+H]⁺: 409.2. Found 409.1.

### Example 78: 5-((3-(1H-Indol-5-yl)-1H-indazol-5-yl)amino)-5,6,7,8-tetrahydronaphthalene-2-carbonitrile, enantiomer 1

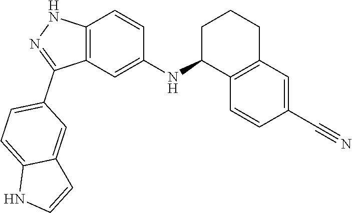 [SMILES: N#Cc1ccc2c(c1)CCC[C@@H]2Nc1ccc2[nH]nc(-c3ccc4[nH]ccc4c3)c2c1]

Prepared as described for 5-((3-(cyclohex-1-en-1-yl)-1H-indazol-5-yl)amino)-5,6,7,8-tetrahydronaphthalene-2-carbonitrile, enantiomer 1, using (1H-indol-5-yl)boronic acid in place of cyclohex-1-en-1-ylboronic acid to afford the title compound (3.99 mg, 16%) as a white solid. ¹H NMR (400 MHz, DMSO-d₆) δ 12.56 (br s, 1H), 11.15 (br s, 1H), 7.97 (s, 1H), 7.62-7.67 (m, 2H), 7.58 (s, 2H), 7.47 (d, J=8.56 Hz, 1H), 7.30-7.39 (m, 2H), 7.12 (s, 1H), 6.98 (br d, J=8.80 Hz, 1H), 6.48 (d, J=2.32 Hz, 1H), 5.83 (br d, J=8.93 Hz, 1H), 4.73 (br d, J=5.99 Hz, 1H), 2.76-2.88 (m, 2H), 1.77-2.00 (m, 4H). MS-ESI (m/z) calc'd for C₂₆H₂₂N₅ [M+H]⁺: 404.2. Found 404.1.

### Example 79: 5-((3-(1H-Pyrrol-2-yl)-1H-indazol-5-yl)amino)-5,6,7,8-tetrahydronaphthalene-2-carbonitrile, enantiomer 1

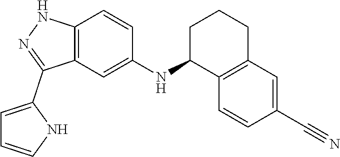 [SMILES: N#Cc1ccc2c(c1)CCC[C@@H]2Nc1ccc2[nH]nc(-c3ccc[nH]3)c2c1]

Prepared as described for 5-((3-(cyclohex-1-en-1-yl)-1H-indazol-5-yl)amino)-5,6,7,8-tetrahydronaphthalene-2-carbonitrile, enantiomer 1, using (1-(tert-butoxycarbonyl)-1H-pyrrol-2-yl)boronic acid in place of cyclohex-1-en-1-ylboronic acid to afford the title compound (4.80 mg, 21%, TFA salt) as a green solid. ¹H NMR (400 MHz, DMSO-d₆) δ 12.53 (s, 1H), 11.16 (br s, 1H), 7.64 (s, 1H), 7.53-7.60 (m, 2H), 7.30 (d, J=8.80 Hz, 1H), 7.04 (s, 1H), 6.96 (dd, J=8.86, 1.77 Hz, 1H), 6.78 (br s, 1H), 6.52 (br s, 1H), 6.13 (q, J=2.61 Hz, 1H), 5.77 (d, J=9.05 Hz, 1H), 4.78 (br d, J=8.07 Hz, 1H), 2.75-2.91 (m, 2H), 1.77-2.04 (m, 4H). MS-ESI (m/z) calc'd for C₂₂H₂₀N₅ [M+H]⁺: 354.2. Found 354.1.

### Example 80: tert-Butyl 2-(5-((6-cyano-1,2,3,4-tetrahydronaphthalen-1-yl)amino)-1H-indazol-3-yl)-1H-pyrrole-1-carboxylate, enantiomer 1

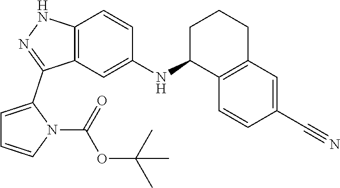 [SMILES: CC(C)(C)OC(=O)n1cccc1-c1n[nH]c2ccc(N[C@H]3CCCc4cc(C#N)ccc43)cc12]

Prepared as described for 5-((3-(cyclohex-1-en-1-yl)-1H-indazol-5-yl)amino)-5,6,7,8-tetrahydronaphthalene-2-carbonitrile, enantiomer 1, using (1-(tert-butoxycarbonyl)-1H-pyrrol-2-yl)boronic acid in place of cyclohex-1-en-1-ylboronic acid to afford the title compound (14.1 mg, 64%) as a white solid. ¹H NMR (400 MHz, DMSO-d₆) δ 12.68 (s, 1H), 7.61 (s, 1H), 7.52-7.57 (m, 1H), 7.45-7.50 (m, 1H), 7.41 (dd, J=2.81, 1.71 Hz, 1H), 7.31 (d, J=8.80 Hz, 1H), 6.95 (dd, J=8.93, 1.59 Hz, 1H), 6.59 (s, 1H), 6.30-6.39 (m, 2H), 5.73 (br d, J=9.17 Hz, 1H), 4.55-4.67 (m, 1H), 2.69-2.89 (m, 2H), 1.70-1.93 (m, 4H), 1.15 (s, 9H). MS-ESI (m/z) calc'd for C₂₇H₂₈N₅O₂ [M+H]⁺: 454.2. Found 454.1.

### Example 81: 5-((3-(4-(Morpholine-4-carbonyl)phenyl)-1H-indazol-5-yl)amino)-5,6,7,8-tetrahydronaphthalene-2-carbonitrile, enantiomer 1

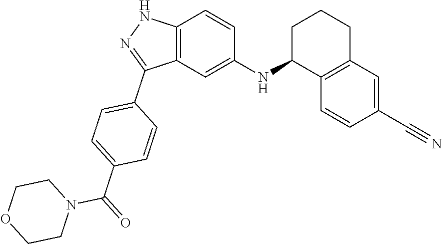 [SMILES: N#Cc1ccc2c(c1)CCC[C@@H]2Nc1ccc2[nH]nc(-c3ccc(C(=O)N4CCOCC4)cc3)c2c1]

Prepared as described for 5-((3-(cyclohex-1-en-1-yl)-1H-indazol-5-yl)amino)-5,6,7,8-tetrahydronaphthalene-2-carbonitrile, enantiomer 1, using morpholino-[4-(4,4,5,5-tetramethyl-1,3,2-dioxaborolan-2-yl)phenyl]methanone in place of cyclohex-1-en-1-ylboronic acid to afford the title compound (7.65 mg, 32%) as a yellow solid. ¹H NMR (400 MHz, DMSO-d₆) δ 12.96 (s, 1H), 7.96 (d, J=8.16 Hz, 2H), 7.64 (s, 1H), 7.47-7.60 (m, 4H), 7.37 (d, J=9.04 Hz, 1H), 7.13 (s, 1H), 6.99 (dd, J=1.65, 8.93 Hz, 1H), 5.89 (d, J=9.04 Hz, 1H), 4.79 (br d, J=7.28 Hz, 1H), 3.38-3.74 (m, 8H), 2.73-2.90 (m, 2H), 1.72-2.02 (m, 4H). MS-ESI (m/z) calc'd for C₂₉H₂₈N₅O₂ [M+H]⁺: 478.2. Found 478.2.

### Example 82: 5-((3-(2-Oxoindolin-6-yl)-1H-indazol-5-yl)amino)-5,6,7,8-tetrahydronaphthalene-2-carbonitrile, enantiomer 1

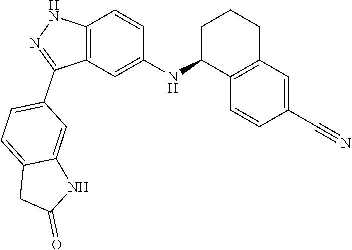 [SMILES: N#Cc1ccc2c(c1)CCC[C@@H]2Nc1ccc2[nH]nc(-c3ccc4c(c3)NC(=O)C4)c2c1]

Prepared as described for 5-((3-(cyclohex-1-en-1-yl)-1H-indazol-5-yl)amino)-5,6,7,8-tetrahydronaphthalene-2-carbonitrile, enantiomer 1, using 6-(4,4,5,5-tetramethyl-1,3,2-dioxaborolan-2-yl)indolin-2-one in place of cyclohex-1-en-1-ylboronic acid to afford the title compound (1.41 mg, 6%) as a pale yellow solid. ¹H NMR (400 MHz, DMSO-d₆) δ 12.82 (s, 1H), 10.42 (s, 1H), 7.64 (s, 1H), 7.51-7.60 (m, 2H), 7.42 (d, J=6.84 Hz, 1H), 7.33-7.37 (m, 2H), 7.28 (d, J=7.72 Hz, 1H), 7.06 (s, 1H), 6.98 (d, J=10.58 Hz, 1H), 5.87 (d, J=8.82 Hz, 1H), 4.72 (s, 1H), 3.51 (s, 2H), 2.83 (br s, 2H), 1.76-2.00 (m, 4H). MS-ESI (m/z) calc'd for C₂₆H₂₂N₅O [M+H]⁺: 420.2. Found 420.1.

### Example 83: 5-((2′-Methyl-1H,2′H-[3,5′-biindazol]-5-yl)amino)-5,6,7,8-tetrahydronaphthalene-2-carbonitrile, enantiomer 1

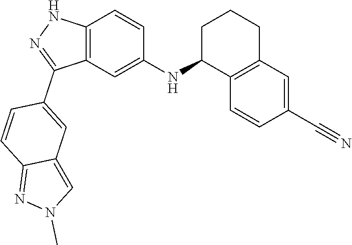 [SMILES: Cn1cc2cc(-c3n[nH]c4ccc(N[C@H]5CCCc6cc(C#N)ccc65)cc34)ccc2n1]

Prepared as described for 5-((3-(cyclohex-1-en-1-yl)-1H-indazol-5-yl)amino)-5,6,7,8-tetrahydronaphthalene-2-carbonitrile, enantiomer 1, using (2-methyl-2H-indazol-5-yl)boronic acid in place of cyclohex-1-en-1-ylboronic acid to afford the title compound (1.5 mg, 6%) as a yellow solid. ¹H NMR (400 MHz, MeOD) δ 8.25 (s, 1H), 8.09 (s, 1H), 7.84 (dd, J=1.59, 9.05 Hz, 1H), 7.69 (d, J=9.05 Hz, 1H), 7.62 (d, J=8.07 Hz, 1H), 7.53 (s, 1H), 7.47 (d, J=8.07 Hz, 1H), 7.42 (d, J=8.93 Hz, 1H), 7.19 (s, 1H), 7.06 (dd, J=2.02, 8.99 Hz, 1H), 4.74 (t, J=5.69 Hz, 1H), 4.19-4.29 (m, 3H), 2.79-2.95 (m, 2H), 1.82-2.08 (m, 4H). MS-ESI (m/z) calc'd for C₂₆H₂₃N₆ [M+H]⁺: 419.2. Found 419.1.

### Example 84: 5-((3-(2,3-Dihydrobenzo[b][1,4]dioxin-6-yl)-1H-indazol-5-yl)amino)-5,6,7,8-tetrahydronaphthalene-2-carbonitrile, enantiomer 1

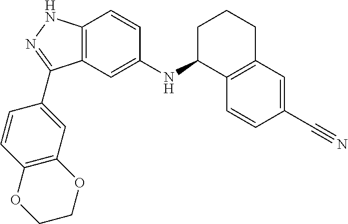 [SMILES: N#Cc1ccc2c(c1)CCC[C@@H]2Nc1ccc2[nH]nc(-c3ccc4c(c3)OCCO4)c2c1]

Prepared as described for 5-((3-(cyclohex-1-en-1-yl)-1H-indazol-5-yl)amino)-5,6,7,8-tetrahydronaphthalene-2-carbonitrile, enantiomer 1, using (2,3-dihydrobenzo[b][1,4]dioxin-6-yl)boronic acid in place of cyclohex-1-en-1-ylboronic acid to afford the title compound (1.7 mg, 6%) as a yellow solid. ¹H NMR (400 MHz, DMSO-d₆) δ 12.79 (br s, 1H), 7.66 (s, 1H), 7.57-7.62 (m, 1H), 7.52-7.57 (m, 1H), 7.28-7.41 (m, 3H), 7.12 (br d, J=11.80 Hz, 1H), 7.02 (br d, J=8.94 Hz, 1H), 6.94 (d, J=8.34 Hz, 1H), 4.77 (br s, 1H), 4.28 (s, 4H), 2.76-2.86 (m, 2H), 1.77-1.93 (m, 4H). MS-ESI (m/z) calc'd for C₂₆H₂₃N₄O₂ [M+H]⁺: 423.2. Found 423.1.

### Example 85: N-(3-(5-((6-Cyano-1,2,3,4-tetrahydronaphthalen-1-yl)amino)-1H-indazol-3-yl)phenyl)acetamide, enantiomer 1

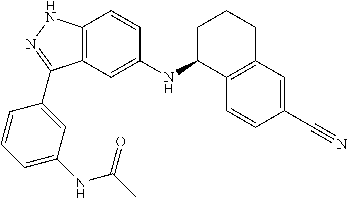 [SMILES: CC(=O)Nc1cccc(-c2n[nH]c3ccc(N[C@H]4CCCc5cc(C#N)ccc54)cc23)c1]

Prepared as described for 5-((3-(cyclohex-1-en-1-yl)-1H-indazol-5-yl)amino)-5,6,7,8-tetrahydronaphthalene-2-carbonitrile, enantiomer 1, using (3-acetamidophenyl)boronic acid in place of cyclohex-1-en-1-ylboronic acid to afford the title compound (8.05 mg, 39%) as a pale yellow solid. ¹H NMR (400 MHz, DMSO-d₆) δ 12.86 (s, 1H), 10.03 (s, 1H), 8.31 (s, 1H), 7.64 (s, 1H), 7.52-7.60 (m, 3H), 7.46 (br d, J=8.46 Hz, 1H), 7.31-7.40 (m, 2H), 7.14 (s, 1H), 6.97 (dd, J=1.55, 8.94 Hz, 1H), 5.89 (d, J=8.94 Hz, 1H), 4.75 (br d, J=8.34 Hz, 1H), 2.77-2.89 (m, 2H), 2.06 (s, 3H), 1.80-2.02 (m, 4H) MS-ESI (m/z) calc'd for C₂₆H₂₄N₅O [M+H]⁺: 422.2. Found 422.1.

### Example 86: 3-(5-((6-Cyano-1,2,3,4-tetrahydronaphthalen-1-yl)amino)-1H-indazol-3-yl)-N,N-dimethylbenzamide, enantiomer 1

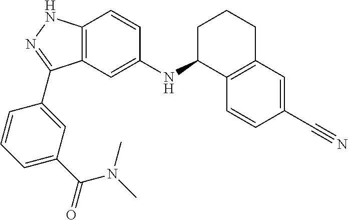 [SMILES: CN(C)C(=O)c1cccc(-c2n[nH]c3ccc(N[C@H]4CCCc5cc(C#N)ccc54)cc23)c1]

Prepared as described for 5-((3-(cyclohex-1-en-1-yl)-1H-indazol-5-yl)amino)-5,6,7,8-tetrahydronaphthalene-2-carbonitrile, enantiomer 1, using (3-(dimethylcarbamoyl)phenyl)boronic acid in place of cyclohex-1-en-1-ylboronic acid to afford the title compound (6.21 mg, 30%) as a gray solid. ¹H NMR (400 MHz, DMSO-d₆) δ 12.92 (s, 1H), 7.94 (d, J=7.94 Hz, 1H), 7.83 (s, 1H), 7.63 (s, 1H), 7.49-7.58 (m, 3H), 7.36 (dd, J=8.27, 14.44 Hz, 2H), 7.02 (dd, J=9.92, 10.80 Hz, 2H), 5.92 (d, J=8.82 Hz, 1H), 4.69-4.76 (m, 1H), 2.90-3.08 (m, 6H), 2.75-2.87 (m, 2H), 1.75-2.02 (m, 4H). MS-ESI (m/z) calc'd for C₂₇H₂₆N₅O [M+H]⁺: 436.2. Found 436.1.

### Example 87: 5-((3-(1,4-Dioxaspiro[4.5]dec-7-en-8-yl)-1H-indazol-5-yl)amino)-5,6,7,8-tetrahydronaphthalene-2-carbonitrile, enantiomer 1

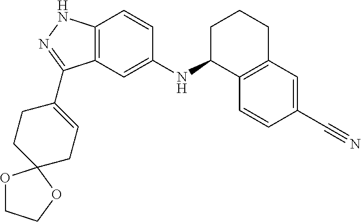 [SMILES: N#Cc1ccc2c(c1)CCC[C@@H]2Nc1ccc2[nH]nc(C3=CCC4(CC3)OCCO4)c2c1]

Prepared as described for 5-((3-(cyclohex-1-en-1-yl)-1H-indazol-5-yl)amino)-5,6,7,8-tetrahydronaphthalene-2-carbonitrile, enantiomer 1, using 4,4,5,5-tetramethyl-2-(1,4-dioxaspiro[4.5]dec-7-en-8-yl)-1,3,2-dioxaborolane in place of cyclohex-1-en-1-ylboronic acid to afford the title compound (7.81 mg, 37%) as a yellow solid. ¹H NMR (400 MHz, DMSO-d₆) δ 12.49 (s, 1H), 7.62 (s, 1H), 7.51-7.58 (m, 2H), 7.27 (d, J=8.60 Hz, 1H), 6.98 (s, 1H), 6.92 (d, J=8.82 Hz, 1H), 6.20 (br s, 1H), 5.72 (d, J=9.04 Hz, 1H), 4.73 (br s, 1H), 3.92 (s, 4H), 2.69-2.89 (m, 4H), 2.41 (br s, 2H), 1.76-1.98 (m, 6H). MS-ESI (m/z) calc'd for C₂₆H₂₇N₄O₂ [M+H]⁺: 427.2. Found 427.1.

### Example 88: 5-((3-(3,6-Dihydro-2H-pyran-4-yl)-1H-indazol-5-yl)amino)-5,6,7,8-tetrahydronaphthalene-2-carbonitrile, enantiomer 1

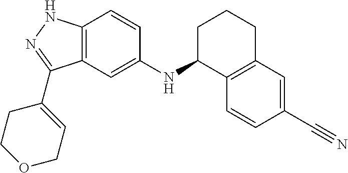 [SMILES: N#Cc1ccc2c(c1)CCC[C@@H]2Nc1ccc2[nH]nc(C3=CCOCC3)c2c1]

Prepared as described for 5-((3-(cyclohex-1-en-1-yl)-1H-indazol-5-yl)amino)-5,6,7,8-tetrahydronaphthalene-2-carbonitrile, enantiomer 1, using 2-(3,6-dihydro-2H-pyran-4-yl)-4,4,5,5-tetramethyl-1,3,2-dioxaborolane in place of cyclohex-1-en-1-ylboronic acid to afford the title compound (3.45 mg, 15%) as a yellow solid. ¹H NMR (400 MHz, DMSO-d₆) δ 12.70 (s, 1H), 7.64 (s, 1H), 7.51-7.61 (m, 2H), 7.33 (br d, J=8.16 Hz, 1H), 7.13 (br s, 1H), 6.97 (br d, J=7.50 Hz, 1H), 6.38 (br s, 1H), 4.79 (br s, 1H), 4.27 (br s, 2H), 3.84 (br t, J=5.18 Hz, 2H), 2.81 (br d, J=8.82 Hz, 2H), 2.62 (br s, 2H), 1.74-1.93 (m, 4H). MS-ESI (m/z) calc'd for C₂₃H₂₃N₄O [M+H]⁺: 371.2. Found 371.1.

### Example 89: 5-((3-(3-Cyanophenyl)-1H-indazol-5-yl)amino)-5,6,7,8-tetrahydronaphthalene-2-carbonitrile, enantiomer 1

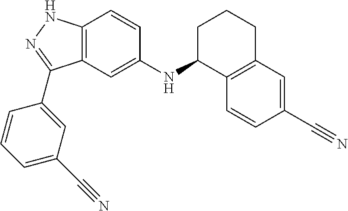 [SMILES: N#Cc1cccc(-c2n[nH]c3ccc(N[C@H]4CCCc5cc(C#N)ccc54)cc23)c1]

Prepared as described for 5-((3-(cyclohex-1-en-1-yl)-1H-indazol-5-yl)amino)-5,6,7,8-tetrahydronaphthalene-2-carbonitrile, enantiomer 1, using (3-cyanophenyl)boronic acid in place of cyclohex-1-en-1-ylboronic acid to afford the title compound (6.8 mg, 28%) as a yellow solid. ¹H NMR (400 MHz, DMSO-d₆) δ 13.15 (br s, 1H), 8.21-8.27 (m, 2H), 7.81 (d, J=7.75 Hz, 1H), 7.68-7.72 (m, 1H), 7.67 (s, 1H), 7.58 (q, J=8.07 Hz, 2H), 7.43 (br d, J=8.70 Hz, 1H), 7.22 (br s, 1H), 7.06 (br d, J=7.75 Hz, 1H), 4.87 (br s, 1H), 2.75-2.92 (m, 2H), 1.75-1.97 (m, 4H). MS-ESI (m/z) calc'd for C₂₅H₂₀N₅ [M+H]⁺: 390.2. Found 390.0.

### Example 90: 5-((3-(2-Methylpyridin-4-yl)-1H-indazol-5-yl)amino)-5,6,7,8-tetrahydronaphthalene-2-carbonitrile, enantiomer 1

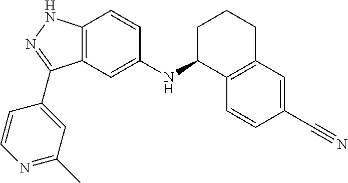 [SMILES: Cc1cc(-c2n[nH]c3ccc(N[C@H]4CCCc5cc(C#N)ccc54)cc23)ccn1]

Prepared as described for 5-((3-(cyclohex-1-en-1-yl)-1H-indazol-5-yl)amino)-5,6,7,8-tetrahydronaphthalene-2-carbonitrile, enantiomer 1, using 2-methyl-4-(4,4,5,5-tetramethyl-1,3,2-dioxaborolan-2-yl)pyridine in place of cyclohex-1-en-1-ylboronic acid to afford the title compound (5.77 mg, 24%) as an orange solid. ¹H NMR (400 MHz, DMSO-d₆) δ 13.92 (br s, 1H), 8.70 (d, J=6.24 Hz, 1H), 8.28-8.33 (m, 2H), 7.66 (s, 1H), 7.57-7.62 (m, 1H), 7.48-7.55 (m, 2H), 7.28 (s, 1H), 7.07 (dd, J=1.71, 9.05 Hz, 1H), 4.96 (br s, 1H), 2.79-2.93 (m, 2H), 2.76 (s, 3H), 1.81-2.04 (m, 4H). MS-ESI (m/z) calc'd for C₂₄H₂₂N₅ [M+H]⁺: 380.2. Found 380.1.

### Example 91: 5-(5-((6-Cyano-1,2,3,4-tetrahydronaphthalen-1-yl)amino)-1H-indazol-3-yl)picolinonitrile, enantiomer 1

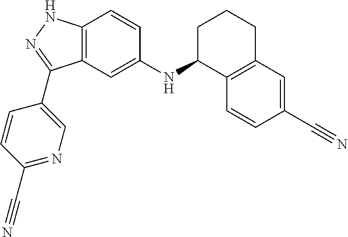 [SMILES: N#Cc1ccc2c(c1)CCC[C@@H]2Nc1ccc2[nH]nc(-c3ccc(C#N)nc3)c2c1]

Prepared as described for 5-((3-(cyclohex-1-en-1-yl)-1H-indazol-5-yl)amino)-5,6,7,8-tetrahydronaphthalene-2-carbonitrile, enantiomer 1, using 5-(4,4,5,5-tetramethyl-1,3,2-dioxaborolan-2-yl)picolinonitrile in place of cyclohex-1-en-1-ylboronic acid to afford the title compound (1.77 mg, 9%) as a yellow gum. ¹H NMR (400 MHz, DMSO-d₆) δ 9.32 (s, 1H), 8.49 (br d, J=9.04 Hz, 1H), 8.08 (d, J=8.38 Hz, 1H), 7.64 (s, 1H), 7.49-7.60 (m, 2H), 7.43 (d, J=9.26 Hz, 1H), 7.20 (s, 1H), 7.01 (d, J=9.04 Hz, 1H), 6.00 (d, J=9.26 Hz, 1H), 4.90 (br s, 1H), 2.82 (br d, J=6.17 Hz, 2H), 1.80-1.99 (m, 4H). MS-ESI (m/z) calc'd for C₂₄H₁₉N₆ [M+H]⁺: 391.2. Found 391.1.

### Example 92: 5-((3-(4-Methoxyphenyl)-1H-indazol-5-yl)amino)-5,6,7,8-tetrahydronaphthalene-2-carbonitrile, enantiomer 1

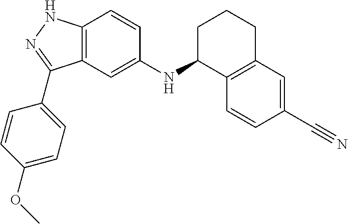 [SMILES: COc1ccc(-c2n[nH]c3ccc(N[C@H]4CCCc5cc(C#N)ccc54)cc23)cc1]

Prepared as described for 5-((3-(cyclohex-1-en-1-yl)-1H-indazol-5-yl)amino)-5,6,7,8-tetrahydronaphthalene-2-carbonitrile, enantiomer 1, using (4-methoxyphenyl)boronic acid in place of cyclohex-1-en-1-ylboronic acid to afford the title compound (10.9 mg, 42%) as a yellow gum. ¹H NMR (400 MHz, DMSO-d₆) δ 7.81 (d, J=8.82 Hz, 2H), 7.68 (s, 1H), 7.59 (q, J=8.23 Hz, 2H), 7.42 (br d, J=8.60 Hz, 1H), 7.29 (br d, J=7.50 Hz, 1H), 7.00-7.13 (m, 3H), 4.83 (br s, 1H), 3.81 (s, 3H), 2.75-2.90 (m, 2H), 1.75-1.95 (m, 4H). MS-ESI (m/z) calc'd for C₂₅H₂₃N₄O [M+H]⁺: 395.2. Found 395.1.

### Example 93: (E)-5-((3-Styryl-1H-indazol-5-yl)amino)-5,6,7,8-tetrahydronaphthalene-2-carbonitrile, enantiomer 1

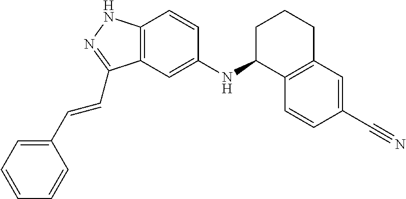 [SMILES: N#Cc1ccc2c(c1)CCC[C@@H]2Nc1ccc2[nH]nc(/C=C/c3ccccc3)c2c1]

Prepared as described for 5-((3-(cyclohex-1-en-1-yl)-1H-indazol-5-yl)amino)-5,6,7,8-tetrahydronaphthalene-2-carbonitrile, enantiomer 1, using (E)-4,4,5,5-tetramethyl-2-styryl-1,3,2-dioxaborolane in place of cyclohex-1-en-1-ylboronic acid to afford the title compound (6.25 mg, 25%) as a yellow solid. ¹H NMR (400 MHz, DMSO-d₆) δ 7.57-7.69 (m, 5H), 7.46-7.53 (m, 1H), 7.24-7.43 (m, 6H), 7.04 (br d, J=8.68 Hz, 1H), 4.88 (br s, 1H), 2.73-2.93 (m, 2H), 1.78-2.03 (m, 4H). MS-ESI (m/z) calc'd for C₂₆H₂₃N₄ [M+H]⁺: 391.2. Found 391.1.

### Example 94: 5-((3-(1-Methyl-1,2,3,6-tetrahydropyridin-4-yl)-1H-indazol-5-yl)amino)-5,6,7,8-tetrahydronaphthalene-2-carbonitrile, enantiomer 1

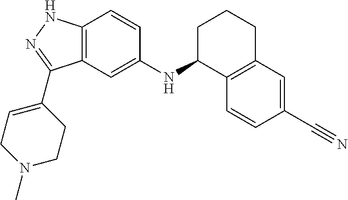 [SMILES: CN1CC=C(c2n[nH]c3ccc(N[C@H]4CCCc5cc(C#N)ccc54)cc23)CC1]

Prepared as described for 5-((3-(cyclohex-1-en-1-yl)-1H-indazol-5-yl)amino)-5,6,7,8-tetrahydronaphthalene-2-carbonitrile, enantiomer 1, using 1-methyl-4-(4,4,5,5-tetramethyl-1,3,2-dioxaborolan-2-yl)-1,2,3,6-tetrahydropyridine in place of cyclohex-1-en-1-ylboronic acid to afford the title compound (9.47 mg, 39%) as a yellow gum. ¹H NMR (400 MHz, DMSO-d₆) δ 12.85 (br s, 1H), 9.85 (br s, 1H), 7.64 (s, 1H), 7.56-7.61 (m, 1H), 7.47-7.55 (m, 1H), 7.34 (d, J=8.93 Hz, 1H), 7.08 (br s, 1H), 6.97 (br d, J=9.05 Hz, 1H), 6.38 (br s, 1H), 4.80 (br d, J=4.65 Hz, 1H), 4.03 (br d, J=16.63 Hz, 1H), 3.82 (br d, J=15.04 Hz, 1H), 3.63 (br d, J=6.97 Hz, 1H), 3.22-3.35 (m, 1H), 2.97-3.09 (m, 1H), 2.70-2.96 (m, 6H), 1.73-1.98 (m, 4H). MS-ESI (m/z) calc'd for C₂₄H₂₆N₅ [M+H]⁺: 384.2. Found 384.1.

### Example 95: 5-((3-(4-Bromophenyl)-1H-indazol-5-yl)amino)-5,6,7,8-tetrahydronaphthalene-2-carbonitrile, enantiomer 1

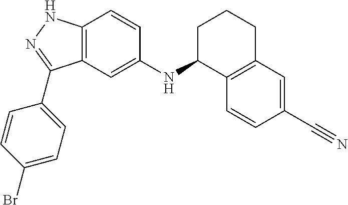 [SMILES: N#Cc1ccc2c(c1)CCC[C@@H]2Nc1ccc2[nH]nc(-c3ccc(Br)cc3)c2c1]

Prepared as described for 5-((3-(cyclohex-1-en-1-yl)-1H-indazol-5-yl)amino)-5,6,7,8-tetrahydronaphthalene-2-carbonitrile, enantiomer 1, using 2-(4-bromophenyl)-4,4,5,5-tetramethyl-1,3,2-dioxaborolane in place of cyclohex-1-en-1-ylboronic acid to afford the title compound (1.85 mg, 2%) as a brown solid. ¹H NMR (400 MHz, DMSO-d₆) δ 7.85 (d, 2H, J=8.6 Hz), 7.6-7.7 (m, 3H), 7.5-7.6 (m, 2H), 7.39 (d, 1H, J=8.6 Hz), 7.1-7.2 (m, 1H), 7.0-7.1 (m, 1H), 4.8-4.8 (m, 1H), 2.8-2.9 (m, 2H), 1.8-2.0 (m, 4H). MS-ESI (m/z) calc'd for C₂₄H₂₀BrN₄ [M+H]⁺: 443.1/445.1. Found 443.0/445.0.

### Example 96: 5-((3-(5,6-Dihydro-4H-pyrrolo[1,2-b]pyrazol-3-yl)-1H-indazol-5-yl)amino)-5,6,7,8-tetrahydronaphthalene-2-carbonitrile, enantiomer 1

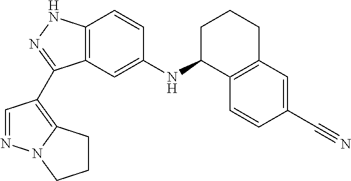 [SMILES: N#Cc1ccc2c(c1)CCC[C@@H]2Nc1ccc2[nH]nc(-c3cnn4c3CCC4)c2c1]

Prepared as described for 5-((3-(cyclohex-1-en-1-yl)-1H-indazol-5-yl)amino)-5,6,7,8-tetrahydronaphthalene-2-carbonitrile, enantiomer 1, using 3-(4,4,5,5-tetramethyl-1,3,2-dioxaborolan-2-yl)-5,6-dihydro-4H-pyrrolo[1,2-b]pyrazole in place of cyclohex-1-en-1-ylboronic acid to afford the title compound (3.50 mg, 14%) as a white solid. ¹H NMR (400 MHz, DMSO-d₆) δ 7.94 (s, 1H), 7.66 (s, 1H), 7.5-7.6 (m, 2H), 7.36 (br d, 1H, J=7.3 Hz), 7.0-7.3 (m, 2H), 4.82 (br s, 1H), 4.1-4.2 (m, 2H), 3.0-3.1 (m, 2H), 2.7-2.9 (m, 2H), 2.60 (quin, 2H, J=7.3 Hz), 1.8-1.9 (m, 4H). MS-ESI (m/z) calc'd for C₂₄H₂₃N₆ [M+H]⁺: 395.2. Found 395.1.

### Example 97: 5-((3-(1-Methyl-2-oxo-1,2-dihydropyridin-4-yl)-1H-indazol-5-yl)amino)-5,6,7,8-tetrahydronaphthalene-2-carbonitrile, enantiomer 1

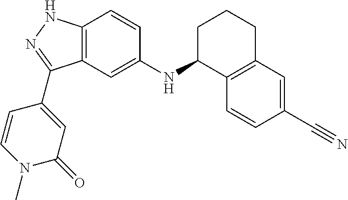 [SMILES: Cn1ccc(-c2n[nH]c3ccc(N[C@H]4CCCc5cc(C#N)ccc54)cc23)cc1=O]

Prepared as described for 5-((3-(cyclohex-1-en-1-yl)-1H-indazol-5-yl)amino)-5,6,7,8-tetrahydronaphthalene-2-carbonitrile, enantiomer 1, using (1-methyl-2-oxo-1,2-dihydropyridin-4-yl)boronic acid in place of cyclohex-1-en-1-ylboronic acid to afford the title compound (2.28 mg, 12%) as a yellow solid. ¹H NMR (400 MHz, DMSO-d₆) δ 13.23 (br s, 1H), 7.7-7.7 (m, 1H), 7.63 (s, 1H), 7.5-7.6 (m, 2H), 7.40 (d, 1H, J=8.9 Hz), 7.09 (s, 1H), 7.01 (dd, 1H, J=1.8, 9.0 Hz), 6.8-6.8 (m, 2H), 6.00 (d, 1H, J=9.0 Hz), 4.7-4.8 (m, 1H), 3.44 (s, 3H), 2.7-2.9 (m, 2H), 1.7-2.0 (m, 4H). MS-ESI (m/z) calc'd for C₂₄H₂₂N₅O [M+H]⁺: 396.2. Found 396.1.

### Example 98: 5-((3-(Pyridin-3-yl)-1H-indazol-5-yl)amino)-5,6,7,8-tetrahydronaphthalene-2-carbonitrile, enantiomer 1

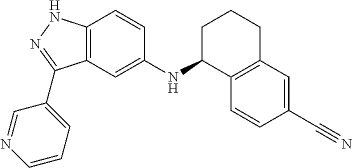 [SMILES: N#Cc1ccc2c(c1)CCC[C@@H]2Nc1ccc2[nH]nc(-c3cccnc3)c2c1]

Prepared as described for 5-((3-(cyclohex-1-en-1-yl)-1H-indazol-5-yl)amino)-5,6,7,8-tetrahydronaphthalene-2-carbonitrile, enantiomer 1, using pyridin-3-ylboronic acid in place of cyclohex-1-en-1-ylboronic acid to afford the title compound (4.84 mg, 21%) as a yellow gum. ¹H NMR (400 MHz, DMSO-d₆) δ 13.27 (br s, 1H), 9.22 (br s, 1H), 8.67 (br s, 1H), 8.59 (br d, J=8.19 Hz, 1H), 7.77 (br d, J=4.65 Hz, 1H), 7.65 (s, 1H), 7.52-7.61 (m, 2H), 7.44 (d, J=8.93 Hz, 1H), 7.22 (br s, 1H), 7.05 (br d, J=8.93 Hz, 1H), 4.87 (br s, 1H), 2.75-2.91 (m, 2H), 1.81-2.00 (m, 4H). MS-ESI (m/z) calc'd for C₂₃H₂₀N₅ [M+H]⁺: 366.2. Found 366.1.

### Example 99: (Z)-5-((3-(Prop-1-en-1-yl)-1H-indazol-5-yl)amino)-5,6,7,8-tetrahydronaphthalene-2-carbonitrile, enantiomer 1

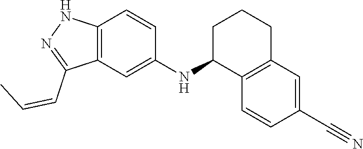 [SMILES: C/C=C\c1n[nH]c2ccc(N[C@H]3CCCc4cc(C#N)ccc43)cc12]

Prepared as described for 5-((3-(cyclohex-1-en-1-yl)-1H-indazol-5-yl)amino)-5,6,7,8-tetrahydronaphthalene-2-carbonitrile, enantiomer 1, using (Z)-prop-1-en-1-ylboronic acid in place of cyclohex-1-en-1-ylboronic acid to afford the title compound (2.11 mg, 10%) as a green solid. ¹H NMR (400 MHz, DMSO-d₆) δ 12.3-12.6 (m, 1H), 7.62 (s, 1H), 7.5-7.6 (m, 2H), 7.25 (d, 1H, J=8.8 Hz), 7.0-7.0 (m, 1H), 6.91 (dd, 1H, J=1.6, 8.9 Hz), 6.63 (dd, 1H, J=1.5, 16.2 Hz), 6.3-6.5 (m, 1H), 5.6-5.8 (m, 1H), 4.6-4.8 (m, 1H), 2.7-2.9 (m, 2H), 1.7-2.0 (m, 7H). MS-ESI (m/z) calc'd for C₂₁H₂₁N₄ [M+H]⁺: 329.2. Found 329.1.

### Example 100: 5-((3-(5-Methylpyridin-3-yl)-1H-indazol-5-yl)amino)-5,6,7,8-tetrahydronaphthalene-2-carbonitrile, enantiomer 1

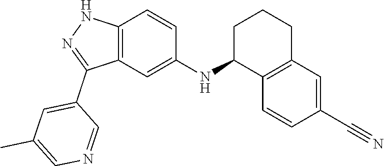 [SMILES: Cc1cncc(-c2n[nH]c3ccc(N[C@H]4CCCc5cc(C#N)ccc54)cc23)c1]

Prepared as described for 5-((3-(cyclohex-1-en-1-yl)-1H-indazol-5-yl)amino)-5,6,7,8-tetrahydronaphthalene-2-carbonitrile, enantiomer 1, using (5-methylpyridin-3-yl)boronic acid in place of cyclohex-1-en-1-ylboronic acid to afford the title compound (2.46 mg, 10%) as a yellow gum. ¹H NMR (400 MHz, DMSO-d₆) δ 13.32 (br s, 1H), 9.08 (s, 1H), 8.59 (s, 1H), 8.50 (s, 1H), 7.65 (s, 1H), 7.5-7.6 (m, 2H), 7.44 (d, 1H, J=9.0 Hz), 7.20 (s, 1H), 7.06 (dd, 1H, J=1.6, 9.0 Hz), 4.8-4.9 (m, 1H), 2.7-2.9 (m, 2H), 1.8-2.0 (m, 4H). MS-ESI (m/z) calc'd for C₂₄H₂₂N₅ [M+H]⁺: 380.2. Found 380.1.

### Example 101: 1-Methoxy-5-{[3-(1,3-oxazol-5-yl)-1H-indazol-5-yl]amino}-5,6,7,8-tetrahydronaphthalene-2-carbonitrile, enantiomer 1 and 2

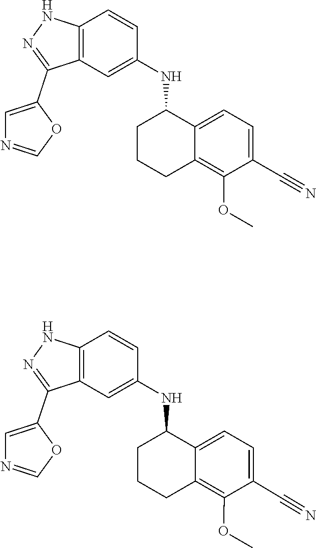 [SMILES: COc1c(C#N)ccc2c1CCC[C@@H]2Nc1ccc2[nH]nc(-c3cnco3)c2c1 ; COc1c(C#N)ccc2c1CCC[C@H]2Nc1ccc2[nH]nc(-c3cnco3)c2c1]

### Step 1: 5-Nitro-1-((2-(trimethylsilyl)ethoxy)methyl)-1H-indazole-3-carbaldehyde

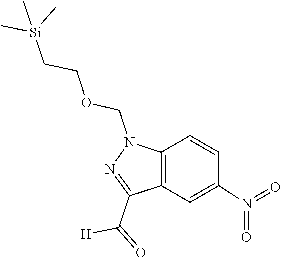 [SMILES: C[Si](C)(C)CCOCn1nc(C=O)c2cc([N+](=O)[O-])ccc21]

To a solution of 5-nitro-1H-indazole-3-carbaldehyde (2.87 g, 15 mmol) in DMF (30 mL) were added K₂CO₃ (2.28 g, 16.5 mmol) and SEM-Cl (2.92 mL, 16.5 mmol). The mixture was stirred at 75° C. for 1 hr, then poured into H₂O. The solid that formed was filtered under vacuum to afford the title compound (4.62 g, 96%) as a red solid. ¹H NMR (400 MHz, DMSO-d₆) δ 10.26 (s, 1H), 8.97 (dd, J=2.2, 0.7 Hz, 1H), 8.42 (dd, J=9.2, 2.2 Hz, 1H), 8.17 (dd, J=9.3, 0.7 Hz, 1H), 6.01 (s, 2H), 3.64-3.56 (m, 2H), 0.88-0.80 (m, 2H), −0.10 (s, 9H). MS-ESI (m/z) calc'd for C₁₄H₂₀N₃₀₄Si [M+H]⁺: 322.1. Found 322.1.

### Step 2: 5-(5-Nitro-1-((2-(trimethylsilyl)ethoxy)methyl)-1H-indazol-3-yl)oxazole

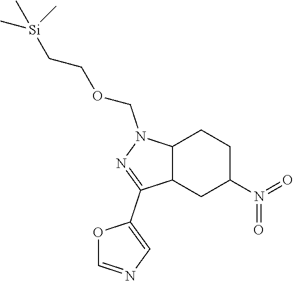 [SMILES: C[Si](C)(C)CCOCN1N=C(c2cnco2)C2CC([N+](=O)[O-])CCC21]

To a suspension of 5-nitro-1-(2-trimethylsilylethoxymethyl)indazole-3-carbaldehyde (4.62 g, 14.37 mmol) in MeOH (71.85 mL) were added K₂CO₃ (2.19 g, 15.81 mmol) and TosMIC (3.09 g, 15.81 mmol). The mixture was then stirred at 65° C. for 2 hrs. The solution was concentrated under vacuum and H₂O was added. The solid that formed was filtered, washed with water and dried to afford the title compound (5.1 g, 98%) as a yellow solid. Major isomer ¹H NMR (400 MHz, DMSO-d₆) δ 8.98 (dd, J=2.2, 0.7 Hz, 1H), 8.68 (s, 1H), 8.39 (dd, J=9.3, 2.2 Hz, 1H), 8.08 (dd, J=9.2, 0.7 Hz, 1H), 8.04 (s, 1H), 5.92 (s, 2H), 3.63-3.56 (m, 2H), 0.86-0.80 (m, 2H), −0.10 (s, 9H). MS-ESI (m/z) calc'd for C₁₆H₂₁N₄O₄Si [M+H]⁺: 361.2. Found 361.1.

### Step 3: 3-(Oxazol-5-yl)-1-((2-(trimethylsilyl)ethoxy)methyl)-1H-indazol-5-amine

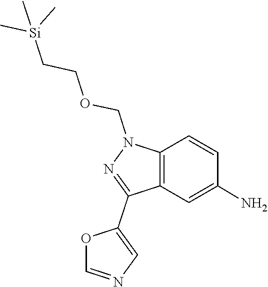 [SMILES: C[Si](C)(C)CCOCn1nc(-c2cnco2)c2cc(N)ccc21]

To a suspension of 5-(5-nitro-1-((2-(trimethylsilyl)ethoxy)methyl)-1H-indazol-3-yl)oxazole (8.7 g, 24.14 mmol) in MeOH (80.46 mL) was added 10% palladium on carbon (1.28 g, 1.21 mmol) and the mixture was hydrogenated at 4 atm for 4 hrs. The catalyst was removed by filtration through Celite and the filtrate was evaporate to dryness. The residue was taken up in water and extracted with EtOAc (2×). The combined organic layers were dried over Na₂SO₄, treated with activated carbon, passed through a phase separator and evaporated to afford the title compound (7.61 g, 95%) as a dark oil. ¹H NMR (400 MHz, DMSO-d₆) δ 8.52 (s, 1H), 7.58 (s, 1H), 7.50 (dd, J=8.9, 0.7 Hz, 1H), 7.06 (dd, J=2.1, 0.7 Hz, 1H), 6.93 (dd, J=8.9, 2.1 Hz, 1H), 5.68 (s, 2H), 5.09 (s, 2H), 3.55-3.50 (m, 2H), 0.87-0.75 (m, 2H), −0.11 (s, 9H). MS-ESI (m/z) calc'd for C₁₆H₂₃N₄₀₂Si [M+H]⁺: 331.2. Found 331.3.

### Step 4: 2-Nitro-N-(3-(oxazol-5-yl)-1-((2-(trimethylsilyl)ethoxy)methyl)-1H-indazol-5-yl)benzenesulfonamide

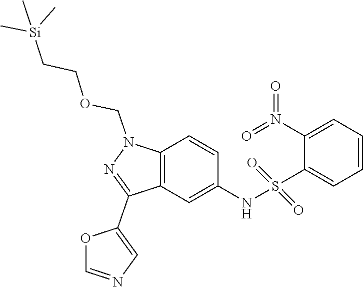 [SMILES: C[Si](C)(C)CCOCn1nc(-c2cnco2)c2cc(NS(=O)(=O)c3ccccc3[N+](=O)[O-])ccc21]

To a solution of 3-(1,3-oxazol-5-yl)-1-(2-trimethylsilylethoxymethyl)indazol-5-amine (7.61 g, 23.03 mmol) in DCM (150 mL) was added pyridine (1.85 mL, 23.03 mmol) and then 2-nitrobenzenesulfonyl chloride (5.1 g, 23.03 mmol). The mixture was stirred at 25° C. for 2 hrs. The solvent was evaporated and the residue was purified by silica gel chromatography using a 0-50% EtOAc/cyclohexane gradient eluent to afford the title compound (8.01 g, 67%) as a yellow solid. ¹H NMR (400 MHz, DMSO-d₆) δ 10.73 (s, 1H), 8.61 (s, 1H), 8.00-7.91 (m, 2H), 7.85-7.72 (m, 4H), 7.65 (s, 1H), 7.29 (dd, J=9.0, 2.0 Hz, 1H), 5.76 (s, 2H), 3.56-3.46 (m, 2H), 0.78 (dd, J=8.5, 7.4 Hz, 2H), −0.15 (s, 9H). MS-ESI (m/z) calc'd for C₂₂H₂₆N₅O₆SSi [M+H]⁺: 516.1. Found 516.1.

### Step 5: N-(6-Cyano-5-methoxy-1,2,3,4-tetrahydronaphthalen-1-yl)-2-nitro-N-(3-(oxazol-5-yl)-1-((2-(trimethylsilyl)ethoxy)methyl)-1H-indazol-5-yl)benzenesulfonamide

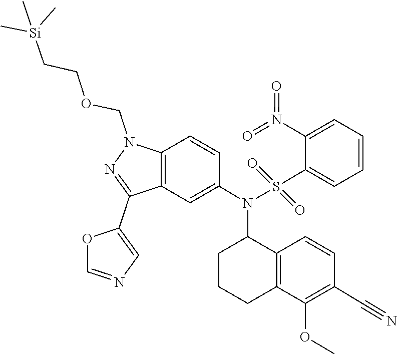 [SMILES: COc1c(C#N)ccc2c1CCCC2N(c1ccc2c(c1)c(-c1cnco1)nn2COCC[Si](C)(C)C)S(=O)(=O)c1ccccc1[N+](=O)[O-]]

To a solution of 5-hydroxy-1-methoxy-5,6,7,8-tetrahydronaphthalene-2-carbonitrile (98.0 mg, 0.39 mmol), 2-nitro-N-[3-(1,3-oxazol-5-yl)-1-(2-trimethylsilylethoxymethyl)indazol-5-yl]benzenesulfonamide (198.9 mg, 0.39 mmol) and triphenylphosphine (202.4 mg, 0.77 mmol) in THF (7 mL) was added dropwise diethyl azodicarboxylate (121.48 μL, 0.77 mmol) and the mixture was stirred at 25° C. for 1.5 hrs. The reaction mixture was partitioned between H₂O and EtOAc, the phases were separated, the aqueous layer was extracted with EtOAc (3×), and the combined organic phases were washed with brine, dried over anhydrous Na₂SO₄ and evaporated to dryness to afford the title compound (360 mg, 67%) as a beige solid. MS-ESI (m/z) calc'd for C₃₄H₃₇N₆O₇SSi [M+H]⁺: 701.2. Found 701.3.

### Step 6: 1-Methoxy-5-((3-(oxazol-5-yl)-1-((2-(trimethylsilyl)ethoxy)methyl)-1H-indazol-5-yl)amino)-5,6,7,8-tetrahydronaphthalene-2-carbonitrile

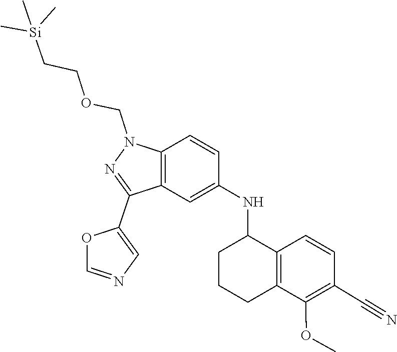 [SMILES: COc1c(C#N)ccc2c1CCCC2Nc1ccc2c(c1)c(-c1cnco1)nn2COCC[Si](C)(C)C]

To a solution of N-(6-cyano-5-methoxy-1,2,3,4-tetrahydronaphthalen-1-yl)-2-nitro-N-[3-(1,3-oxazol-5-yl)-1-(2-trimethylsilylethoxymethyl)indazol-5-yl]benzenesulfonamide (360.0 mg, 0.26 mmol) in DMF (5 mL) was added K₂CO₃ (142 mg, 1.03 mmol) and benzenethiol (0.08 mL, 0.77 mmol), the mixture was stirred at r.t. for 1 hr. The reaction mixture was partitioned between H₂O and EtOAc and the phases were separated. The aqueous layer was extracted with EtOAc (2×) and the combined organic phases were washed with brine (1×), dried over anhydrous Na₂SO₄, and evaporated to dryness. The residue was purified by SCX, using a 5 g cartridge, washed with MeOH and eluted the compound with NH₃ 2M solution in MeOH. Product-containing fractions were combined and evaporated to dryness to afford the title compound (128 mg, 63%) as a yellow solid. ¹H NMR (400 MHz, DMSO-d₆) δ 12.42 (br. s., 1H) 7.16-7.21 (m, 1H) 7.01 (t, J=1.98 Hz, 1H) 6.96-7.00 (m, 1H) 6.42-6.44 (m, 1H) 6.42 (s, 1H) 6.33-6.41 (m, 1H) 6.46 (s, 1H) 3.82-3.84 (m, 1H) 3.82 (s, 1H) 3.81-3.84 (m, 1H) 3.78 (s, 3H) 3.48 (d, J=7.04 Hz, 1H) 3.29-3.33 (m, 1H) 3.31 (br. s., 2H) 3.18 (d, J=5.72 Hz, 2H). MS-ESI (m/z) calc'd for C₂₈H₃₄N₅O₃Si [M+H]⁺: 516.2. Found 516.3.

### Step 7: 1-Methoxy-5-{[3-(1,3-oxazol-5-yl)-1H-indazol-5-yl]amino}-5,6,7,8-tetrahydronaphthalene-2-carbonitrile

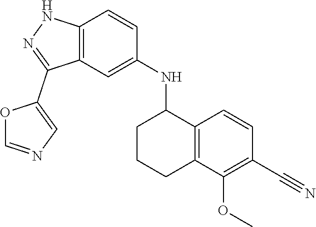 [SMILES: COc1c(C#N)ccc2c1CCCC2Nc1ccc2[nH]nc(-c3cnco3)c2c1]

To a solution of 1-methoxy-5-{[3-(1,3-oxazol-5-yl)-1-{[2-(trimethylsilyl)ethoxy]methyl}-1H-indazol-5-yl]amino}-5,6,7,8-tetrahydronaphthalene-2-carbonitrile (128.0 mg, 0.16 mmol) in DCM (4 mL) was added trifluoroacetic acid (1 mL) and the mixture was stirred at r.t. overnight. The mixture was evaporated to dryness and redissolved in MeOH (4 mL), NH₄OH (1 mL) was added and the mixture was stirred at r.t. for 1 hr. The reaction mixture was then partitioned between H₂O and EtOAc and the phases were separated. The aqueous layer was extracted with EtOAc (3×) and the combined organic phases were washed with H₂O, dried over anhydrous Na₂SO₄ and evaporated to dryness. The residue was purified by reversed phase chromatography using a 0-80% EtOAc/cyclohexane gradient eluent to afford the title compound (59 mg, 95%).

### Step 8: 1-Methoxy-5-{[3-(1,3-oxazol-5-yl)-1H-indazol-5-yl]amino}-5,6,7,8-tetrahydronaphthalene-2-carbonitrile, enantiomer 1 and 2

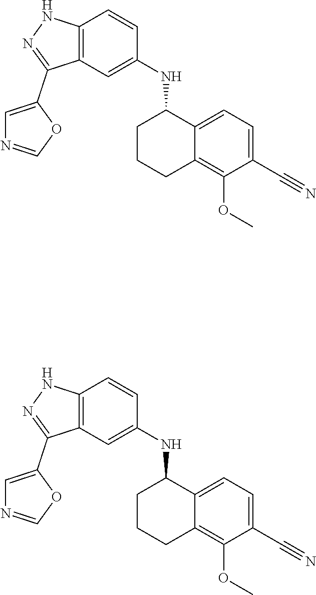 [SMILES: COc1c(C#N)ccc2c1CCC[C@@H]2Nc1ccc2[nH]nc(-c3cnco3)c2c1 ; COc1c(C#N)ccc2c1CCC[C@H]2Nc1ccc2[nH]nc(-c3cnco3)c2c1]

1-Methoxy-5-{[3-(1,3-oxazol-5-yl)-1H-indazol-5-yl]amino}-5,6,7,8-tetrahydronaphthalene-2-carbonitrile was subjected to chiral separation using Method EC to afford 1-methoxy-5-{[3-(1,3-oxazol-5-yl)-1H-indazol-5-yl]amino}-5,6,7,8-tetrahydronaphthalene-2-carbonitrile, enantiomer 1 (15.87 mg, 26%) as an off-white solid. ¹H NMR (400 MHz, DMSO-d₆) δ 13.09 (br. s., 1H) 8.44 (s, 1H) 7.64 (s, 1H) 7.54 (d, J=8.1 Hz, 1H) 7.37 (d, J=8.8 Hz, 1H) 7.29 (d, J=8.14 Hz, 1H) 7.03 (s, 1H) 6.99 (dd, J=9.1, 2.09 Hz, 1H) 5.95 (d, J=9.02 Hz, 1H) 4.81 (brs, 1H) 3.93 (s, 3H) 2.65-2.85 (m, 2H) 1.77-1.99 (m, 4H). MS-ESI (m/z) calc'd for C₂₂H₂₀N₅O₂ [M+H]⁺: 386.1. Found 386.2. A later eluting fraction was also isolated to afford 1-methoxy-5-{[3-(1,3-oxazol-5-yl)-1H-indazol-5-yl]amino}-5,6,7,8-tetrahydronaphthalene-2-carbonitrile, enantiomer 2 (15.98 mg, 26%) as an off-white solid. ¹H NMR (400 MHz, DMSO-d₆) δ 13.08 (br. s., 1H) 8.44 (s, 1H) 7.64 (s, 1H) 7.54 (d, J=7.92 Hz, 1H) 7.37 (d, J=8.80 Hz, 1H) 7.29 (d, J=8.14 Hz, 1H) 7.03 (s, 1H) 6.99 (dd, J=8.91, 2.09 Hz, 1H) 5.95 (d, J=9.24 Hz, 1H) 4.82 (br. s., 1H) 3.93 (s, 3H) 2.65-2.86 (m, 2H) 1.77-1.99 (m, 4H). MS-ESI (m/z) calc'd for C₂₂H₂₀N₅O₂ [M+H]⁺: 386.1. Found 386.2.

### Example 102: 2-Methoxy-8-((3-(oxazol-5-yl)-1H-indazol-5-yl)amino)-5,6,7,8-tetrahydroquinoline-3-carbonitrile, enantiomer 1 and 2

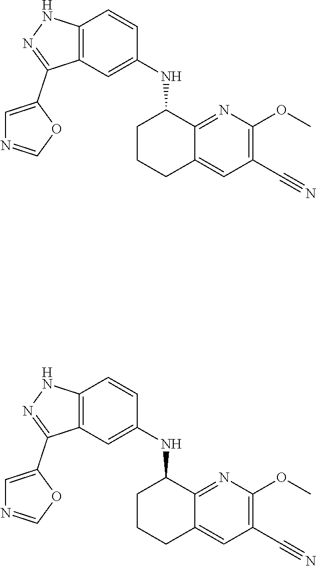 [SMILES: COc1nc2c(cc1C#N)CCC[C@@H]2Nc1ccc2[nH]nc(-c3cnco3)c2c1 ; COc1nc2c(cc1C#N)CCC[C@H]2Nc1ccc2[nH]nc(-c3cnco3)c2c1]

### Step 1: 2-Chloro-3-cyano-5,6,7,8-tetrahydroquinoline 1-oxide

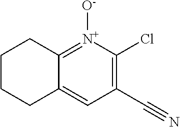 [SMILES: N#Cc1cc2c([n+]([O-])c1Cl)CCCC2]

To a solution of 2-chloro-5,6,7,8-tetrahydroquinoline-3-carbonitrile (1.0 g, 5.19 mmol) in trifluoroacetic acid (25 mL) was added hydrogen peroxide (1.59 mL, 15.57 mmol) and the mixture was stirred at 75° C. for 15 hrs. Water was added and the solution was neutralized by addition of solid K₂CO₃. The mixture was then extracted with DCM (3×) and the combined organic layers were washed with H₂O, passed through a phase separator, and the solvent was evaporated under reduced pressure to afford the title compound (875 mg, 81%) as a light yellow solid. ¹H NMR (400 MHz, DMSO-d₆) δ 7.78 (s, 1H) 2.79 (dt, J=16.67, 6.19 Hz, 4H) 1.77-1.92 (m, 2H) 1.63-1.74 (m, 2H), MS-ESI (m/z) calc'd for C₁₀H₉ClN[N+][O−] [M+H]⁺: 209.0. Found 209.0.

### Step 2: 3-Cyano-2-methoxy-5,6,7,8-tetrahydroquinoline 1-oxide

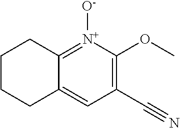

To a solution of 2-chloro-3-cyano-5,6,7,8-tetrahydroquinoline 1-oxide (875.0 mg, 4.19 mmol) in MeOH (12 mL) was added sodium methoxide (453.1 mg, 8.39 mmol) and the mixture was stirred at r.t. for 48 hrs. The reaction mixture was partitioned between H₂O and EtOAc and the phases were separated. The aqueous layer was extracted with EtOAc (2×) and the combined organic phases were washed with brine, dried over anhydrous Na₂SO₄, and evaporated to dryness. The material was purified by silica gel chromatography first using a 0-100% EtOAc/cyclohexane gradient eluent and then with a 90% EtOAc/MeOH eluent to afford the title compound (260 mg, 30%) as a yellow solid. ¹H NMR (400 MHz, DMSO-d₆) δ 7.60 (s, 1H) 4.15 (s, 3H) 2.77 (t, J=6.49 Hz, 2H) 2.72 (t, J=6.16 Hz, 2H) 1.76-1.86 (m, 2H) 1.63-1.72 (m, 2H) MS-ESI (m/z) calc'd for C₁₁H₁₃N[N+]O[O−] [M+H]⁺: 205.1. Found 205.1.

### Step 3: 8-Hydroxy-2-methoxy-5,6,7,8-tetrahydroquinoline-3-carbonitrile

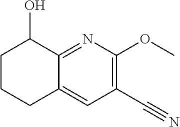

To a solution of 3-cyano-2-methoxy-5,6,7,8-tetrahydroquinoline 1-oxide (260.0 mg, 1.27 mmol) in DCM (5 mL) was added dropwise trifluoroacetic anhydride (0.53 mL, 3.82 mmol) and the mixture was stirred at 25° C. for 1 hr. The solvent was evaporated and the residue was taken up in MeOH, then K₂CO₃ was added to make the solution basic and the suspension was stirred at 25° C. for 20 min. The solvent was evaporated and the residue was taken up in H₂O and extracted with DCM (3×). The combined organic layers were washed with brine, passed through a phase separator and evaporated under reduced pressure to dryness. The material was purified by normal phase chromatography on a 10 g silica gel column, using a 0-40% EtOAc/cyclohexane gradient eluent to afford the title compound (191.4 mg, 74%) as a white solid. ¹H NMR (400 MHz, DMSO-d₆) δ 8.00 (s, 1H) 5.24 (d, J=4.62 Hz, 1H) 4.48 (d, J=5.06 Hz, 1H) 3.99 (s, 3H) 2.55-2.78 (m, 2H) 1.75-1.99 (m, 3H) 1.56-1.74 (m, 1H). MS-ESI (m/z) calc'd for C₁₁H₁₃N₂O₂ [M+H]⁺: 205.1. Found 205.1.

### Step 4: N-(3-Cyano-2-methoxy-5,6,7,8-tetrahydroquinolin-8-yl)-2-nitro-N-(3-(oxazol-5-yl)-1-((2-(trimethylsilyl)ethoxy)methyl)-1H-indazol-5-yl)benzenesulfonamide

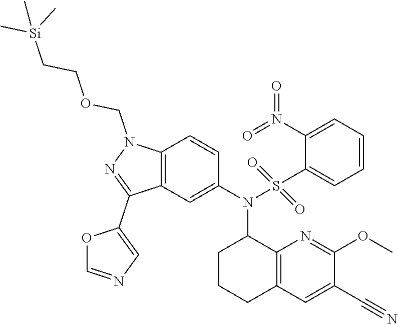

To a solution of 8-hydroxy-2-methoxy-5,6,7,8-tetrahydroquinoline-3-carbonitrile (100.0 mg, 0.49 mmol), 2-nitro-N-[3-(1,3-oxazol-5-yl)-1-(2-trimethylsilylethoxymethyl)indazol-5-yl]benzenesulfonamide (252.5 mg, 0.49 mmol) and triphenylphosphine (256.9 mg, 0.98 mmol) in THF (7 mL), was added dropwise diethyl azodicarboxylate (154.2 μL, 0.98 mmol) and the mixture was stirred at 25° C. for 1.5 hrs. The reaction mixture was partitioned between H₂O and EtOAc and the phases were separated. The aqueous layer was extracted with EtOAc (2×) and the combined organic phases were washed with H₂O, dried over anhydrous Na₂SO₄, filtered, and evaporated to dryness. The residue was purified by chromatography on a 25 g silica gel column, using a 0-50% EtOAc/cyclohexane gradient eluent to afford the title compound (429 mg, 85%) as a beige solid. ¹H NMR (400 MHz, DMSO-d₆) δ 8.50 (s, 1H) 8.01 (dd, J=8.03, 0.99 Hz, 1H) 7.97 (s, 1H) 7.87-7.93 (m, 1H) 7.69-7.80 (m, 3H) 7.56 (d, J=1.54 Hz, 1H) 7.53 (s, 1H) 7.06 (dd, J=9.02, 1.76 Hz, 1H) 5.76 (s, 2H) 5.53-5.62 (m, 1H) 4.02 (s, 3H) 3.52 (t, J=8.03 Hz, 2H) 2.27-2.48 (m, 2H) 1.59-1.94 (m, 4H) 0.77 (td, J=7.98, 1.43 Hz, 2H) −0.17-−0.15 (m, 9H). MS-ESI (m/z) calc'd for C₃₃H₃₆N₇₀₇SSi [M+H]⁺: 702.2. Found 702.3.

### Step 5: 2-Methoxy-8-((3-(oxazol-5-yl)-1-((2-(trimethylsilyl)ethoxy)methyl)-1H-indazol-5-yl)amino)-5,6,7,8-tetrahydroquinoline-3-carbonitrile

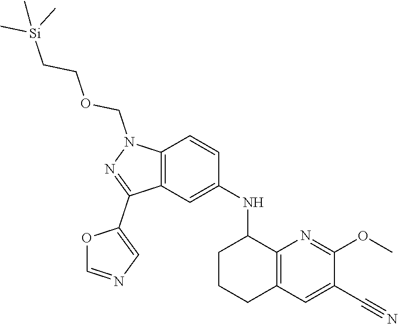

To a solution of N-(3-cyano-2-methoxy-5,6,7,8-tetrahydroquinolin-8-yl)-2-nitro-N-[3-(1,3-oxazol-5-yl)-1-(2-trimethylsilylethoxymethyl)indazol-5-yl]benzenesulfonamide (343.9 mg, 0.49 mmol) in DMF (5 mL) were added K₂CO₃ (270.9 mg, 1.96 mmol) and benzenethiol (0.15 mL, 1.47 mmol) and the mixture was stirred at r.t. for 1 hr. The reaction mixture was partitioned between water and EtOAc and the phases were separated. The aqueous layer was extracted with EtOAc (2×) and the combined organic phases were washed with water (1×), dried over anhydrous Na₂SO₄, filtered, and evaporated to dryness. The residue was purified by SCX, using a 5 g cartridge, washing with MeOH and eluting the compound with a 2 M solution of NH₃ in MeOH to afford the title compound (165 mg, 65%) as a yellow solid. ¹H NMR (400 MHz, DMSO-d₆) δ 8.51 (s, 1H) 8.05 (s, 1H) 7.71 (s, 1H) 7.54 (d, J=9.02 Hz, 1H) 7.25 (s, 1H) 7.11 (dd, J=9.02, 1.76 Hz, 1H) 5.94 (d, J=8.14 Hz, 1H) 5.70 (s, 2H) 4.74 (d, J=7.04 Hz, 1H) 3.64 (s, 3H) 3.53 (t, J=7.92 Hz, 2H) 2.69-2.82 (m, 2H) 2.03-2.14 (m, 1H) 1.88-2.00 (m, 2H) 1.75-1.87 (m, 1H) 0.80 (t, J=8.03 Hz, 2H) −0.11 (s, 9H). MS-ESI (m/z) calc'd for C₂₇H₃₃N₆O₃Si [M+H]⁺: 517.2. Found 517.2.

### Step 6: 2-Methoxy-8-((3-(oxazol-5-yl)-1H-indazol-5-yl)amino)-5,6,7,8-tetrahydroquinoline-3-carbonitrile

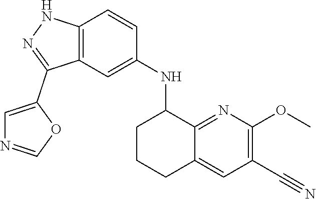 [SMILES: COc1nc2c(cc1C#N)CCCC2Nc1ccc2[nH]nc(-c3cnco3)c2c1]

Prepared as described for 1-methoxy-5-{[3-(1,3-oxazol-5-yl)-1H-indazol-5-yl]amino}-5,6,7,8-tetrahydronaphthalene-2-carbonitrile using 2-methoxy-8-{[3-(1,3-oxazol-5-yl)-1-{[2-(trimethylsilyl)ethoxy]methyl}-1H-indazol-5-yl]amino}-5,6,7,8-tetrahydroquinoline-3-carbonitrile in place of 1-methoxy-5-{[3-(1,3-oxazol-5-yl)-1-{[2-(trimethylsilyl)ethoxy]methyl}-1H-indazol-5-yl]amino}-5,6,7,8-tetrahydronaphthalene-2-carbonitrile, to afford the title compound (57 mg, 46%).

### Step 7: 2-Methoxy-8-((3-(oxazol-5-yl)-1H-indazol-5-yl)amino)-5,6,7,8-tetrahydroquinoline-3-carbonitrile, enantiomer 1 and 2

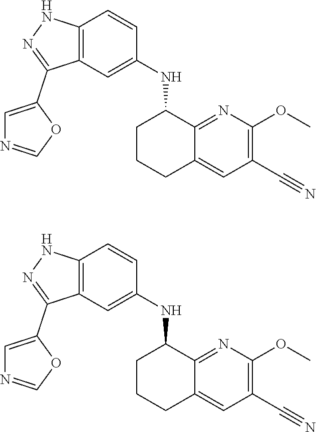 [SMILES: COc1nc2c(cc1C#N)CCC[C@@H]2Nc1ccc2[nH]nc(-c3cnco3)c2c1 ; COc1nc2c(cc1C#N)CCC[C@H]2Nc1ccc2[nH]nc(-c3cnco3)c2c1]

2-Methoxy-8-((3-(oxazol-5-yl)-1H-indazol-5-yl)amino)-5,6,7,8-tetrahydroquinoline-3-carbonitrile was subjected to chiral separation using Method EB to afford 2-methoxy-8-((3-(oxazol-5-yl)-1H-indazol-5-yl)amino)-5,6,7,8-tetrahydroquinoline-3-carbonitrile, enantiomer 1 (19.8 mg, 16%) as a yellow solid. ¹H NMR (400 MHz, DMSO-d₆) δ 13.06 (br. s., 1H) 8.46 (s, 1H) 8.05 (s, 1H) 7.62 (s, 1H) 7.35 (d, J=9.02 Hz, 1H) 7.21 (s, 1H) 7.04 (dd, J=8.80, 1.98 Hz, 1H) 5.83 (d, J=7.48 Hz, 1H) 4.63-4.78 (m, 1H) 3.68 (s, 3H) 2.68-2.82 (m, 2H) 2.01-2.18 (m, 1H) 1.88-2.00 (m, 2H) 1.76-1.86 (m, 1H). MS-ESI (m/z) calc'd for C₂₁H₁₉N₆O₂ [M+H]⁺: 387.2. Found 387.2. A later eluting fraction was also isolated to afford 2-methoxy-8-((3-(oxazol-5-yl)-1H-indazol-5-yl)amino)-5,6,7,8-tetrahydroquinoline-3-carbonitrile, enantiomer 2 (21.8 mg, 18%) as a yellow solid. ¹H NMR (400 MHz, DMSO-d₆) δ 13.06 (br. s., 1H) 8.46 (s, 1H) 8.05 (s, 1H) 7.63 (s, 1H) 7.35 (d, J=9.02 Hz, 1H) 7.21 (d, J=1.54 Hz, 1H) 7.04 (dd, J=9.02, 1.98 Hz, 1H) 5.83 (d, J=7.70 Hz, 1H) 4.63-4.79 (m, 1H) 3.68 (s, 3H) 2.69-2.84 (m, 2H) 2.01-2.15 (m, 1H) 1.90-2.00 (m, 2H) 1.77-1.86 (m, 1H). MS-ESI (m/z) calc'd for C₂₁H₁₉N₆O₂ [M+H]⁺: 387.2. Found 387.2.

### Example 103: 7-((3-(Oxazol-5-yl)-1H-indazol-5-yl)oxy)-6,7-dihydro-5H-cyclopenta[b]pyridine-3-carbonitrile, enantiomer 1 and 2

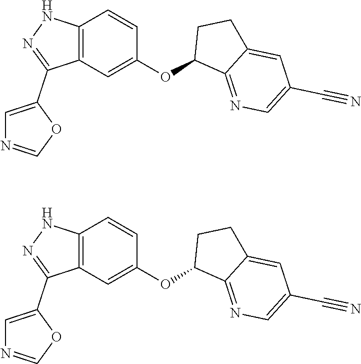 [SMILES: N#Cc1cnc2c(c1)CC[C@@H]2Oc1ccc2[nH]nc(-c3cnco3)c2c1 ; N#Cc1cnc2c(c1)CC[C@H]2Oc1ccc2[nH]nc(-c3cnco3)c2c1]

### Step 1: 5-Bromo-1-((2-(trimethylsilyl)ethoxy)methyl)-1H-indazole-3-carbaldehyde

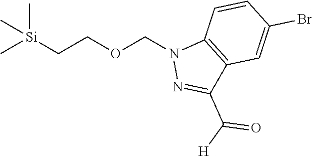 [SMILES: C[Si](C)(C)CCOCn1nc(C=O)c2cc(Br)ccc21]

To a solution of 5-bromo-1H-indazole-3-carbaldehyde (4.91 g, 21.82 mmol) in DMF (52.32 mL) was added K₂CO₃ (3.32 g, 24 mmol) and SEM-Cl (4.25 mL, 24 mmol). The mixture was stirred at 25° C. for 1 hr, then poured into H₂O and extracted with Et₂O. The combined organic layers were passed through a phase separator and evaporated to afford the title compound (7.75 g, 95%) as a red oil. ¹H NMR (400 MHz, DMSO-d₆) δ 10.19 (s, 1H), 8.30 (dd, J=1.9, 0.7 Hz, 1H), 7.92 (dd, J=8.9, 0.7 Hz, 1H), 7.74 (dd, J=8.9, 1.9 Hz, 1H), 5.92 (s, 2H), 3.60-3.54 (m, 2H), 0.83-0.77 (m, 2H), −0.11 (s, 9H). MS-ESI (m/z) calc'd for C₁₄H₄₀BrN₂O₂Si [M+H]⁺: 355.0. Found 355.1, 357.1.

### Step 2: 5-(5-Bromo-1-((2-(trimethylsilyl)ethoxy)methyl)-1H-indazol-3-yl)oxazole

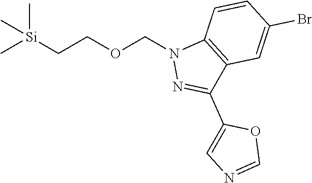 [SMILES: C[Si](C)(C)CCOCn1nc(-c2cnco2)c2cc(Br)ccc21]

To a suspension of 5-bromo-1-((2-(trimethylsilyl)ethoxy)methyl)-1H-indazole-3-carbaldehyde (7.75 g, 21.82 mmol) in MeOH (109.1 mL) was added K₂CO₃ (3.32 g, 24 mmol) and TosMIC (4.69 g, 24 mmol), then the mixture was stirred at 65° C. for 1 hr. The solvent was evaporated and the residue was taken up in water and extracted with DCM. The organic layer was passed through a phase separator and evaporated to obtain a dark oil which was purified by column chromatography (NH, 100 g cartridge), using a 0-30% EtOAc/cyclohexane gradient eluent to afford the title compound (6.62 g, 77%) as an orange oil. ¹H NMR (400 MHz, DMSO-d₆) δ 8.57 (s, 1H), 8.31 (dd, J=1.8, 0.7 Hz, 1H), 7.96 (s, 1H), 7.84 (dd, J=8.9, 0.7 Hz, 1H), 7.68 (dd, J=8.9, 1.8 Hz, 1H), 5.83 (s, 2H), 3.58-3.52 (m, 2H), 0.81 (dd, J=8.4, 7.5 Hz, 2H), −0.12 (s, 9H). MS-ESI (m/z) calc'd for C₁₆H₂₁BrN₃O₂Si [M+H]⁺: 396.1, 394.1. Found 396.1, 394.1.

### Step 3: 5-(5-(4,4,5,5-Tetramethyl-1,3,2-dioxaborolan-2-yl)-1-((2-(trimethylsilyl)ethoxy)methyl)-1H-indazol-3-yl)oxazole

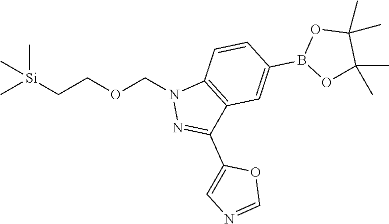

To a solution of 5-(5-bromo-1-((2-(trimethylsilyl)ethoxy)methyl)-1H-indazol-3-yl)oxazole (1.97 g, 5 mmol) in 1,4-dioxane (50 mL) was added KOAc (1.47 g, 15 mmol) and bis(pinacolato)diborane (1.4 g, 5.5 mmol). The mixture was degassed then Pd(dppf)Cl₂.DCM (0.41 g, 0.500 mmol) was added and the reaction was stirred at 100° C. for 4 hrs. The solvent was evaporated and the residue was taken up in DCM and filtered through Celite. The filtrate was evaporated and the residue was purified by silica gel chromatography using a 0-50% EtOAc/cyclohexane gradient to afford the title compound (2.21 g, 100%) as an orange oil. ¹H NMR (400 MHz, DMSO-d₆) δ 8.84-8.59 (m, 1H), 8.45-8.35 (m, 1H), 7.87-7.54 (m, 3H), 5.99-5.79 (m, 2H), 3.68-3.50 (m, 2H), 1.37-1.28 (m, 12H), 0.88-0.75 (m, 2H), −0.08-−0.17 (m, 9H). MS-ESI (m/z) calc'd for C₂₂H₃₃BN₃O₄Si [M+H]⁺: 442.3. Found 442.3, 444.3.

### Step 4: 3-(Oxazol-5-yl)-1-((2-(trimethylsilyl)ethoxy)methyl)-1H-indazol-5-ol

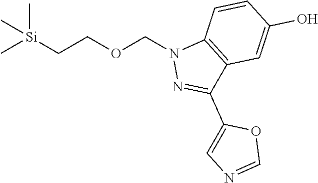 [SMILES: C[Si](C)(C)CCOCn1nc(-c2cnco2)c2cc(O)ccc21]

To a solution of 5-(5-(4,4,5,5-tetramethyl-1,3,2-dioxaborolan-2-yl)-1-((2-(trimethylsilyl)ethoxy)methyl)-1H-indazol-3-yl)oxazole (2.21 g, 5 mmol) in MeOH (25 mL) was added hydrogen peroxide (2.55 mL, 25 mmol) and the mixture was stirred at 25° C. for 4 hrs. The solvent was evaporated and the residue was taken up in H₂O and extracted with EtOAc. The organic layer was dried over Na₂SO₄, filtered, and the solvent evaporated to give a residue which was purified by silica gel chromatography using a 0-50% EtOAc/cyclohexane gradient eluent to afford the title compound (1.32 g, 80%) as a beige solid. ¹H NMR (400 MHz, DMSO-d₆) δ 9.50 (s, 1H), 8.54 (s, 1H), 7.67 (s, 1H), 7.64 (dd, J=9.0, 0.7 Hz, 1H), 7.29 (dd, J=2.2, 0.7 Hz, 1H), 7.07 (dd, J=9.0, 2.2 Hz, 1H), 5.74 (s, 2H), 3.53 (dd, J=8.4, 7.5 Hz, 2H), 0.83-0.78 (m, 2H), −0.11 (s, 9H). MS-ESI (m/z) calc'd for C₁₆H₂₄N₃O₃Si [M+H]⁺: 332.1. Found 332.1.

### Step 5: 5-(5-((3-Bromo-6,7-dihydro-5H-cyclopenta[b]pyridin-7-yl)oxy)-1-((2-(trimethylsilyl)ethoxy)methyl)-1H-indazol-3-yl)oxazole

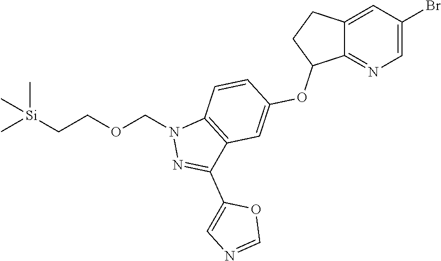 [SMILES: C[Si](C)(C)CCOCn1nc(-c2cnco2)c2cc(OC3CCc4cc(Br)cnc43)ccc21]

To a solution of 3-bromo-5H,6H,7H-cyclopenta[b]pyridin-7-ol (100.0 mg, 0.47 mmol), 3-(oxazol-5-yl)-1-((2-(trimethylsilyl)ethoxy)methyl)-1H-indazol-5-ol (154.84 mg, 0.47 mmol) and triphenylphosphine (245.06 mg, 0.93 mmol) in THF (7.0 mL) was added diethyl azodicarboxylate (0.15 mL, 0.93 mmol) dropwise and the mixture was stirred at 25° C. for 15 hrs. The solvent was evaporated and the residue was purified by silica gel chromatography using a 0-50% EtOAc/cyclohexane gradient eluent to afford the title compound (70 mg, 28%). MS-ESI (m/z) calc'd for C₂₄H₂₈BrN₄O₃Si [M+H]⁺: 527.1, 529.1. Found 527.1, 529.1.

### Step 6: 7-((3-(Oxazol-5-yl)-1-((2-(trimethylsilyl)ethoxy)methyl)-1H-indazol-5-yl)oxy)-6,7-dihydro-5H-cyclopenta[b]pyridine-3-carbonitrile

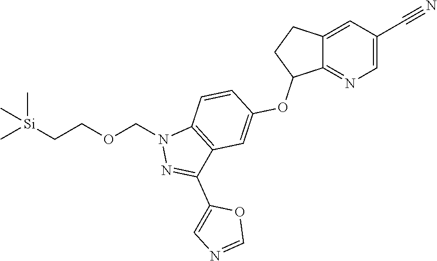 [SMILES: C[Si](C)(C)CCOCn1nc(-c2cnco2)c2cc(OC3CCc4cc(C#N)cnc43)ccc21]

A 0.1 N aqueous solution of potassium hexacyanoferrate (II) (1.33 mL, 0.13 mmol), 5-(5-((3-bromo-6,7-dihydro-5H-cyclopenta[b]pyridin-7-yl)oxy)-1-((2-(trimethylsilyl)ethoxy)methyl)-1H-indazol-3-yl)oxazole (70.0 mg, 0.13 mmol) and KOAc (13.02 mg, 0.13 mmol) were dissolved in a mixture of 1,4-dioxane (1.27 mL)/H₂O (0.13 mL) in a sealed microwave vial. The mixture was degassed with N₂ for 15 minutes, then XPhos (18.49 mg, 0.04 mmol) and XPhos Pd G3 (32.84 mg, 0.04 mmol) were added and the mixture was stirred at 105° C. for 4 hrs. The reaction mixture was partitioned between H₂O and EtOAc and the phases were separated. The aqueous layer was extracted with EtOAc (2×) and the combined organic phases were washed with brine, dried over anhydrous Na₂SO₄, filtered, and evaporated to dryness. The material was purified by silica gel chromatography using a 0-80% EtOAc/cyclohexane gradient eluent to afford the title compound (30 mg, 48%) as an orange solid. MS-ESI (m/z) calc'd for C₂₅H₂₈N₅O₃Si [M+H]⁺: 474.1. Found 474.3.

### Step 7: 7-((3-(Oxazol-5-yl)-1H-indazol-5-yl)oxy)-6,7-dihydro-5H-cyclopenta[b]pyridine-3-carbonitrile

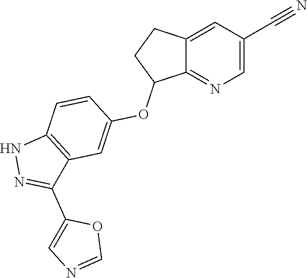 [SMILES: N#Cc1cnc2c(c1)CCC2Oc1ccc2[nH]nc(-c3cnco3)c2c1]

Prepared as described for 1-methoxy-5-{[3-(1,3-oxazol-5-yl)-1H-indazol-5-yl]amino}-5,6,7,8-tetrahydronaphthalene-2-carbonitrile using 7-((3-(oxazol-5-yl)-1-((2-(trimethylsilyl)ethoxy)methyl)-1H-indazol-5-yl)oxy)-6,7-dihydro-5H-cyclopenta[b]pyridine-3-carbonitrile in place of 1-methoxy-5-{[3-(1,3-oxazol-5-yl)-1-{[2-(trimethylsilyl)ethoxy]methyl}-1H-indazol-5-yl]amino}-5,6,7,8-tetrahydronaphthalene-2-carbonitrile, to afford the title compound (19.8 mg, 16%) as an orange solid.

### Step 8: 7-((3-(Oxazol-5-yl)-1H-indazol-5-yl)oxy)-6,7-dihydro-5H-cyclopenta[b]pyridine-3-carbonitrile, enantiomer 1 and 2

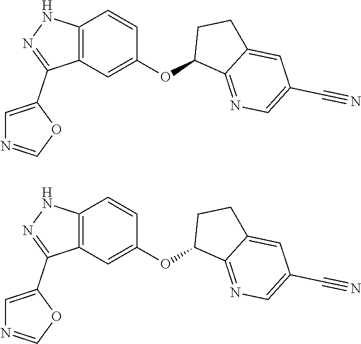 [SMILES: N#Cc1cnc2c(c1)CC[C@@H]2Oc1ccc2[nH]nc(-c3cnco3)c2c1 ; N#Cc1cnc2c(c1)CC[C@H]2Oc1ccc2[nH]nc(-c3cnco3)c2c1]

7-((3-(Oxazol-5-yl)-1H-indazol-5-yl)oxy)-6,7-dihydro-5H-cyclopenta[b]pyridine-3-carbonitrile was purified by chiral separation using Method CM to afford 7-((3-(oxazol-5-yl)-1H-indazol-5-yl)oxy)-6,7-dihydro-5H-cyclopenta[b]pyridine-3-carbonitrile, enantiomer 1 (3.4 mg, 16%) as a white solid. ¹H NMR (400 MHz, MeOD) δ 8.85-8.74 (m, 1H), 8.36 (s, 1H), 8.16 (dd, J=2.0, 1.0 Hz, 1H), 7.75 (d, J=2.3 Hz, 1H), 7.69 (s, 1H), 7.53 (dd, J=9.0, 0.7 Hz, 1H), 7.24 (dd, J=9.0, 2.3 Hz, 1H), 5.91 (dd, J=7.0, 4.5 Hz, 1H), 3.27-3.19 (m, 1H), 3.15-2.99 (m, 1H), 2.83-2.69 (m, 1H), 2.40-2.28 (m, 1H). MS-ESI (m/z) calc'd for C₂₁H₂₀N₆O₂ [M+H]⁺: 344.1. Found 344.2. A later eluting fraction was also isolated to afford 7-((3-(oxazol-5-yl)-1H-indazol-5-yl)oxy)-6,7-dihydro-5H-cyclopenta[b]pyridine-3-carbonitrile, enantiomer 2 (3.0 mg, 14%) as a white solid. ¹H NMR (400 MHz, MeOD) δ 8.80 (dd, J=1.9, 0.9 Hz, 1H), 8.36 (s, 1H), 8.16 (dd, J=2.0, 1.0 Hz, 1H), 7.75 (d, J=2.3 Hz, 1H), 7.68 (s, 1H), 7.53 (dd, J=9.1, 0.7 Hz, 1H), 7.24 (dd, J=9.1, 2.3 Hz, 1H), 5.90 (dd, J=7.0, 4.5 Hz, 1H), 3.28-3.18 (m, 1H), 3.15-3.01 (m, 1H), 2.83-2.69 (m, 1H), 2.42-2.27 (m, 1H). MS-ESI (m/z) calc'd for C₂₁H₂₀N₆O₂ [M+H]⁺: 344.1. Found 344.2.

### Example 104: 5-((3-(Oxazol-5-yl)-1H-indazol-5-yl)amino)-6,7,8,9-tetrahydro-5H-benzo[7]annulene-2-carbonitrile, enantiomer 1 and 2

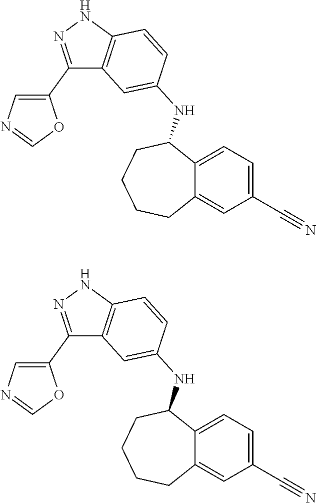 [SMILES: N#Cc1ccc2c(c1)CCCC[C@@H]2Nc1ccc2[nH]nc(-c3cnco3)c2c1 ; N#Cc1ccc2c(c1)CCCC[C@H]2Nc1ccc2[nH]nc(-c3cnco3)c2c1]

### Step 1: 2-Bromo-6,7,8,9-tetrahydro-5H-benzo[7]annulen-5-ol

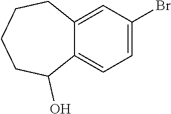 [SMILES: OC1CCCCc2cc(Br)ccc21]

To a solution of 2-bromo-6,7,8,9-tetrahydro-5H-benzo[7]annulen-5-one (300.0 mg, 1.25 mmol) in MeOH (5 mL) was added sodium borohydride (71.2 mg, 1.88 mmol) and the mixture was stirred at r.t. for 1 hr. The reaction was quenched with water and then extracted with EtOAc. The organic phase was dried over anhydrous Na₂SO₄ and evaporated to dryness. The residue was purified by silica gel chromatography using a 0-30% EtOAc/cyclohexane gradient eluent to afford the title compound (260 mg, 86%) as a colorless oil. ¹H NMR (400 MHz, DMSO-d₆) δ 7.38 (d, J=8.2 Hz, 1H), 7.33 (dd, J=8.2, 2.1 Hz, 1H), 7.28 (d, J=2.1 Hz, 1H), 5.27 (d, J=4.2 Hz, 1H), 4.75-4.62 (m, 1H), 2.80 (dd, J=13.9, 7.3 Hz, 1H), 2.72-2.59 (m, 1H), 1.88 (d, J=11.3 Hz, 2H), 1.81-1.58 (m, 2H), 1.45 (q, J=11.8, 10.7 Hz, 1H), 1.24 (t, J=12.1 Hz, 1H) MS-ESI (m/z) calc'd for C₁₁H₁₄BrO [M+H]⁺: 241.0, 242.0. Found 223.0, 224.9 [M+H-OH]⁺.

### Step 2: N-(2-Bromo-6,7,8,9-tetrahydro-5H-benzo[7]annulen-5-yl)-2-nitro-N-(3-(oxazol-5-yl)-1-((2-(trimethylsilyl)ethoxy)methyl)-1H-indazol-5-yl)benzenesulfonamide

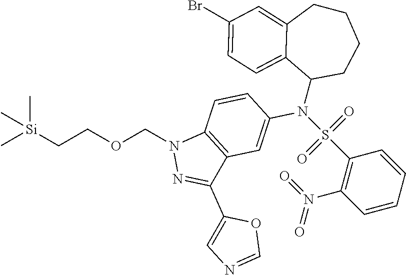 [SMILES: C[Si](C)(C)CCOCn1nc(-c2cnco2)c2cc(N(C3CCCCc4cc(Br)ccc43)S(=O)(=O)c3ccccc3[N+](=O)[O-])ccc21]

To a solution of 2-bromo-6,7,8,9-tetrahydro-5H-benzo[7]annulen-5-ol (90.0 mg, 0.37 mmol), 2-nitro-N-[3-(1,3-oxazol-5-yl)-1-(2-trimethylsilylethoxymethyl)indazol-5-yl]benzenesulfonamide (192.46 mg, 0.37 mmol) and triphenylphosphine (195.8 mg, 0.75 mmol) in THF (2.4 mL) was added diethyl azodicarboxylate (0.12 mL, 0.75 mmol) dropwise and the mixture was stirred at 25° C. for 18 hrs. After evaporation of the solvent, the residue was extracted with EtOAc and H₂O. The organic phase was dried over Na₂SO₄, filtered, and concentrated to dryness. The residue was purified by reversed phase chromatography using a 2-100% MeCN—H₂O (0.1% HCOOH) gradient eluent to afford the title compound (152 mg, 55%) as a yellow solid. MS-ESI (m/z) calc'd for C₃₃H₃₇BrN₅O₆SSi [M+H]⁺: 738.1, Found 738.2, 740.2.

### Step 3: N-(2-Cyano-6,7,8,9-tetrahydro-5H-benzo[7]annulen-5-yl)-2-nitro-N-(3-(oxazol-5-yl)-1-((2-(trimethylsilyl)ethoxy)methyl)-1H-indazol-5-yl)benzenesulfonamide

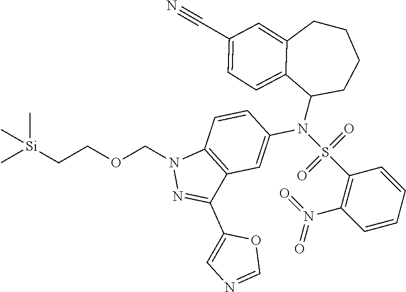 [SMILES: C[Si](C)(C)CCOCn1nc(-c2cnco2)c2cc(N(C3CCCCc4cc(C#N)ccc43)S(=O)(=O)c3ccccc3[N+](=O)[O-])ccc21]

A 0.1 N aqueous solution of potassium hexacyanoferrate (II) (2.03 mL, 0.20 mmol), N-(2-bromo-6,7,8,9-tetrahydro-5H-benzo[7]annulen-5-yl)-2-nitro-N-(3-(oxazol-5-yl)-1-((2-(trimethylsilyl)ethoxy)methyl)-1H-indazol-5-yl)benzenesulfonamide (150.0 mg, 0.20 mmol) and KOAc (19.93 mg, 0.20 mmol) were dissolved in a mixture of 1,4-dioxane (2.0 mL)/H₂O (0.2 mL) in a sealed MW vial. The mixture was degassed with N₂ for 15 minutes. Then XPhos (18.49 mg, 0.040 mmol) and XPhos Pd G3 (13.75 mg, 0.020 mmol) were added and the mixture was left stirring at 105° C. for 2 hrs. The reaction mixture was partitioned between H₂O and EtOAc and the phases were separated. The aqueous layer was extracted with EtOAc (2×) and the combined organic phases were washed with brine, dried over anhydrous Na₂SO₄, filtered, and evaporated to dryness. The material was purified by silica gel chromatography using a 0-50% EtOAc/cyclohexane gradient eluent to afford the title compound (58 mg, 42%). MS-ESI (m/z) calc'd for C₃₄H₃₇N₆O₆SSi [M+H]⁺: 685.2, Found 685.2.

### Step 4: 5-((3-(Oxazol-5-yl)-1-((2-(trimethylsilyl)ethoxy)methyl)-1H-indazol-5-yl)amino)-6,7,8,9-tetrahydro-5H-benzo[7]annulene-2-carbonitrile

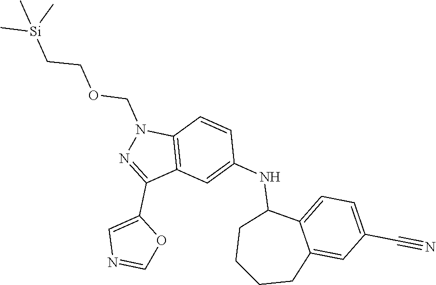 [SMILES: C[Si](C)(C)CCOCn1nc(-c2cnco2)c2cc(NC3CCCCc4cc(C#N)ccc43)ccc21]

To a solution of N-(2-cyano-6,7,8,9-tetrahydro-5H-benzo[7]annulen-5-yl)-2-nitro-N-(3-(oxazol-5-yl)-1-((2-(trimethylsilyl)ethoxy)methyl)-1H-indazol-5-yl)benzenesulfonamide (58.0 mg, 0.08 mmol) in DMF (0.862 mL) was added K₂CO₃ (46.82 mg, 0.34 mmol) and benzenethiol (0.03 mL, 0.250 mmol). The mixture was then stirred at 25° C. for 2 hrs. Water was added and the mixture was extracted with EtOAc (3×). The combined organic layers were evaporated to give a yellow oil which was passed through an SCX cartridge to afford the title compound (36 mg, 85%) as a yellow oil. MS-ESI (m/z) calc'd for C₂₈H₃₄N₅O₂Si [M+H]⁺: 500.2, Found 500.0.

### Step 5: 5-((3-(Oxazol-5-yl)-1H-indazol-5-yl)amino)-6,7,8,9-tetrahydro-5H-benzo[7]annulene-2-carbonitrile

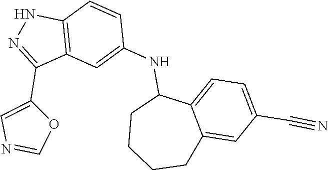 [SMILES: N#Cc1ccc2c(c1)CCCCC2Nc1ccc2[nH]nc(-c3cnco3)c2c1]

Prepared as described for 1-methoxy-5-{[3-(1,3-oxazol-5-yl)-1H-indazol-5-yl]amino}-5,6,7,8-tetrahydronaphthalene-2-carbonitrile using 5-((3-(oxazol-5-yl)-1-((2-(trimethylsilyl)ethoxy)methyl)-1H-indazol-5-yl)amino)-6,7,8,9-tetrahydro-5H-benzo[7]annulene-2-carbonitrile in place of 1-methoxy-5-{[3-(1,3-oxazol-5-yl)-1-{[2-(trimethylsilyl)ethoxy]methyl}-1H-indazol-5-yl]amino}-5,6,7,8-tetrahydronaphthalene-2-carbonitrile, to afford the title compound (7.0 mg, 42%).

### Step 6: 5-((3-(Oxazol-5-yl)-1H-indazol-5-yl)amino)-6,7,8,9-tetrahydro-5H-benzo[7]annulene-2-carbonitrile, enantiomer 1 and 2

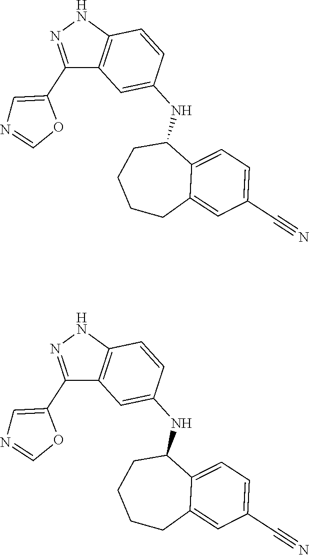 [SMILES: N#Cc1ccc2c(c1)CCCC[C@@H]2Nc1ccc2[nH]nc(-c3cnco3)c2c1 ; N#Cc1ccc2c(c1)CCCC[C@H]2Nc1ccc2[nH]nc(-c3cnco3)c2c1]

5-((3-(Oxazol-5-yl)-1H-indazol-5-yl)amino)-6,7,8,9-tetrahydro-5H-benzo[7]annulene-2-carbonitrile was subjected to chiral separation using Method CO to afford 5-{[3-(1,3-oxazol-5-yl)-1H-indazol-5-yl]amino}-6,7,8,9-tetrahydro-5H-benzo[7]annulene-2-carbonitrile, enantiomer 1 (3.5 mg, 21%) as a white solid. ¹H NMR (400 MHz, MeOD) δ 8.23 (s, 1H), 7.54 (d, J=1.7 Hz, 1H), 7.51 (d, J=8.0 Hz, 1H), 7.43 (dd, J=8.0, 1.8 Hz, 1H), 7.36 (dd, J=9.0, 0.7 Hz, 1H), 7.29 (s, 1H), 7.02 (dd, J=9.0, 2.1 Hz, 1H), 6.61 (d, J=2.1 Hz, 1H), 4.74 (d, J=9.7 Hz, 1H), 3.25-3.14 (m, 1H), 2.99 (dd, J=14.8, 5.5 Hz, 1H), 2.17 (d, J=13.3 Hz, 1H), 2.01 (t, J=5.3 Hz, 3H), 1.82-1.66 (m, 1H), 1.51-1.35 (m, 1H). MS-ESI (m/z) calc'd for C₂₂H₂₀N₅O [M+H]⁺: 370.2. Found 370.2. A later eluting fraction was also isolated to afford 5-{[3-(1,3-oxazol-5-yl)-1H-indazol-5-yl]amino}-6,7,8,9-tetrahydro-5H-benzo[7]annulene-2-carbonitrile, enantiomer 2 (3.1 mg, 14%) as a white solid. ¹H NMR (400 MHz, MeOD) δ 8.23 (s, 1H), 7.53 (d, J=1.7 Hz, 1H), 7.50 (d, J=8.0 Hz, 1H), 7.43 (dd, J=8.0, 1.7 Hz, 1H), 7.36 (dd, J=9.0, 0.7 Hz, 1H), 7.29 (s, 1H), 7.02 (dd, J=9.0, 2.2 Hz, 1H), 6.61 (d, J=2.3 Hz, 1H), 4.74 (dd, J=9.8, 1.9 Hz, 1H), 3.26-3.13 (m, 1H), 3.06-2.91 (m, 1H), 2.26-2.12 (m, 1H), 2.10-1.94 (m, 3H), 1.83-1.66 (m, 1H), 1.53-1.35 (m, 1H). MS-ESI (m/z) calc'd for C₂₂H₂₀N₅O [M+H]⁺: 370.2. Found 370.2.

### Example 105: 3,3-Dimethyl-1-((3-(oxazol-5-yl)-1H-indazol-5-yl)amino)-2,3-dihydro-1H-indene-5-carbonitrile, enantiomer 1 and 2

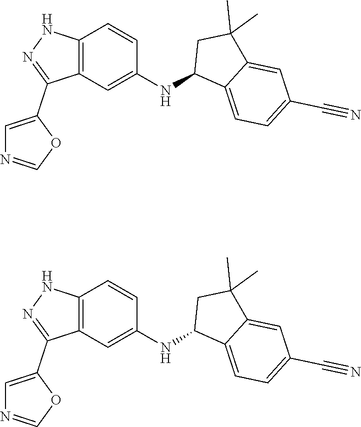 [SMILES: CC1(C)C[C@H](Nc2ccc3[nH]nc(-c4cnco4)c3c2)c2ccc(C#N)cc21 ; CC1(C)C[C@@H](Nc2ccc3[nH]nc(-c4cnco4)c3c2)c2ccc(C#N)cc21]

### Step 1: 3,3-Dimethyl-1-oxo-2,3-dihydro-1H-indene-5-carbonitrile

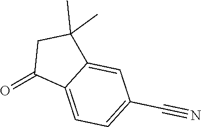

To a solution of 3,3-dimethyl-1-oxo-2H-indene-5-carbonitrile (70.0 mg, 0.380 mmol) in MeOH (3.5 mL) was added sodium borohydride (20.02 mg, 0.530 mmol) and the mixture was stirred at r.t. for 1 hr. The solvent was evaporated and the residue was taken up in EtOAc and H₂O. The organic phase was dried over Na₂SO₄, filtered, and concentrated to dryness. The residue was purified by silica gel chromatography using a 0-50% EtOAc/cyclohexane gradient eluent to afford the title compound (54 mg, 59%) as a pale yellow solid. ¹H NMR (400 MHz, DMSO-d₆) δ 7.72 (d, J=1.5 Hz, 1H), 7.66 (dd, J=7.8, 1.5 Hz, 1H), 7.47 (d, J=7.8 Hz, 1H), 5.48 (d, J=5.7 Hz, 1H), 5.14 (q, J=6.8 Hz, 1H), 2.26 (dd, J=12.6, 7.1 Hz, 1H), 1.73 (dd, J=12.6, 7.4 Hz, 1H), 1.34 (s, 3H), 1.16 (s, 3H). MS-ESI (m/z) calc'd for C₁₂H₁₄NO [M+H]⁺: 188.2, Found 188.1.

### Step 2: 1-Hydroxy-3,3-dimethyl-2,3-dihydro-1H-indene-5-carbonitrile

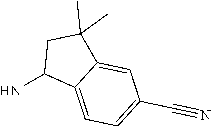

To a solution of 3,3-dimethyl-1-oxo-2H-indene-5-carbonitrile (70.0 mg, 0.380 mmol) in MeOH (3.5 mL) was added sodium borohydride (20.02 mg, 0.530 mmol) and the mixture was stirred at r.t. for 1 hr. The solvent was evaporated and the residue was extracted with EtOAc and H₂O. The organic phase was dried over Na₂SO₄, filtered, and concentrated to dryness. The residue was purified by silica gel chromatography using using a 0-50% EtOAc/cyclohexane gradient eluent to afford the title compound (54 mg, 59%). ¹H NMR (400 MHz, DMSO-d₆) δ 7.72 (d, J=1.5 Hz, 1H), 7.66 (dd, J=7.8, 1.5 Hz, 1H), 7.47 (d, J=7.8 Hz, 1H), 5.48 (d, J=5.7 Hz, 1H), 5.14 (q, J=6.8 Hz, 1H), 2.26 (dd, J=12.6, 7.1 Hz, 1H), 1.73 (dd, J=12.6, 7.4 Hz, 1H), 1.34 (s, 3H), 1.16 (s, 3H). MS-ESI (m/z) calc'd for C₁₂H₁₄NO [M+H]⁺: 188.2, Found 188.1.

### Step 3: N-(5-Cyano-3,3-dimethyl-2,3-dihydro-1H-inden-1-yl)-2-nitro-N-(3-(oxazol-5-yl)-1-((2-(trimethylsilyl)ethoxy)methyl)-1H-indazol-5-yl)benzenesulfonamide

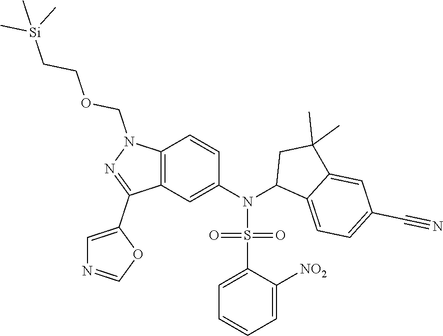 [SMILES: CC1(C)CC(N(c2ccc3c(c2)c(-c2cnco2)nn3COCC[Si](C)(C)C)S(=O)(=O)c2ccccc2[N+](=O)[O-])c2ccc(C#N)cc21]

To a solution of 1-hydroxy-3,3-dimethyl-2,3-dihydro-1H-indene-5-carbonitrile (54.0 mg, 0.29 mmol), 2-nitro-N-[3-(1,3-oxazol-5-yl)-1-(2-trimethylsilylethoxymethyl)indazol-5-yl]benzenesulfonamide (148.7 mg, 0.29 mmol) and triphenylphosphine (151.3 mg, 0.58 mmol) in THF (2.803 mL), was added dropwise diethyl azodicarboxylate (0.09 mL, 0.58 mmol) and the mixture was stirred at 25° C. for 18 hrs. After evaporation of the solvent, the residue was taken up in EtOAc and H₂O. The organic phase was dried over Na₂SO₄, filtered and concentrated to dryness. The residue was purified by reversed phase chromatography using a 2-100% MeCN—H₂O (0.1% HCOOH) gradient eluent to afford the title compound (165 mg, 84%) as a yellow solid. MS-ESI (m/z) calc'd for C₃₄H₃₇N₆O₆SSi [M+H]⁺: 685.2, Found 685.4.

### Step 4: 3,3-Dimethyl-1-((3-(oxazol-5-yl)-1-((2-(trimethylsilyl)ethoxy)methyl)-1H-indazol-5-yl)amino)-2,3-dihydro-1H-indene-5-carbonitrile

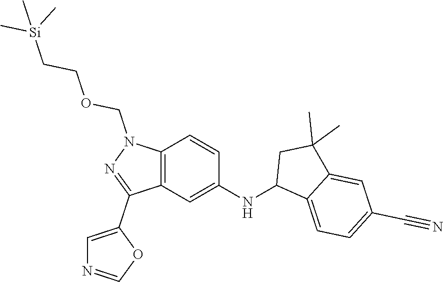 [SMILES: CC1(C)CC(Nc2ccc3c(c2)c(-c2cnco2)nn3COCC[Si](C)(C)C)c2ccc(C#N)cc21]

To a solution of N-(5-cyano-3,3-dimethyl-1,2-dihydroinden-1-yl)-2-nitro-N-[3-(1,3-oxazol-5-yl)-1-(2-trimethylsilylethoxymethyl)indazol-5-yl]benzenesulfonamide (165.0 mg, 0.24 mmol) in DMF (2.45 mL) was added K₂CO₃ (133.2 mg, 0.96 mmol) and benzenethiol (0.07 mL, 0.72 mmol) and the mixture was stirred at 25° C. for 2 hrs. Water was added and the mixture was extracted with EtOAc (3×). The combined organic layers were evaporated to give a yellow oil which was passed through an SCX cartridge to afford the title compound (95 mg, 79%), as a yellow oil. MS-ESI (m/z) calc'd for C₂₈H₃₄N₅O₂Si [M+H]⁺: 500.2, Found 500.3.

### Step 5: 3,3-Dimethyl-1-((3-(oxazol-5-yl)-1H-indazol-5-yl)amino)-2,3-dihydro-1H-indene-5-carbonitrile

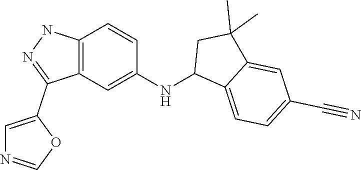 [SMILES: CC1(C)CC(Nc2ccc3[nH]nc(-c4cnco4)c3c2)c2ccc(C#N)cc21]

Prepared as described for 1-methoxy-5-{[3-(1,3-oxazol-5-yl)-1H-indazol-5-yl]amino}-5,6,7,8-tetrahydronaphthalene-2-carbonitrile using 3,3-dimethyl-1-{[3-(1,3-oxazol-5-yl)-1-{[2-(trimethylsilyl)ethoxy]methyl}-1H-indazol-5-yl]amino}-2,3-dihydro-1H-indene-5-carbonitrile in place of 1-methoxy-5-{[3-(1,3-oxazol-5-yl)-1-{[2-(trimethylsilyl)ethoxy]methyl}-1H-indazol-5-yl]amino}-5,6,7,8-tetrahydronaphthalene-2-carbonitrile, to afford the title compound (50.0 mg, 71%) as a yellow solid.

### Step 6: 3,3-Dimethyl-1-((3-(oxazol-5-yl)-1H-indazol-5-yl)amino)-2,3-dihydro-1H-indene-5-carbonitrile, enantiomer 1 and 2

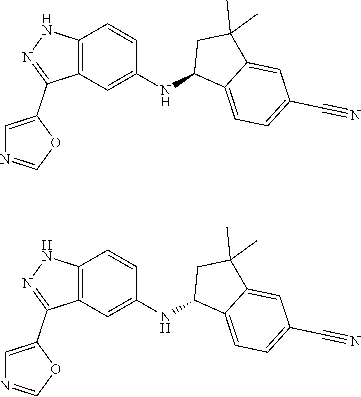 [SMILES: CC1(C)C[C@H](Nc2ccc3[nH]nc(-c4cnco4)c3c2)c2ccc(C#N)cc21 ; CC1(C)C[C@@H](Nc2ccc3[nH]nc(-c4cnco4)c3c2)c2ccc(C#N)cc21]

3,3-Dimethyl-1-((3-(oxazol-5-yl)-1H-indazol-5-yl)amino)-2,3-dihydro-1H-indene-5-carbonitrile was subjected to chiral separation using Method CQ to afford 3,3-dimethyl-1-{[3-(1,3-oxazol-5-yl)-1H-indazol-5-yl]amino}-2,3-dihydro-1H-indene-5-carbonitrile, enantiomer 1 (16.4 mg, 23%) as a white solid. ¹H NMR (400 MHz, DMSO-d₆) δ 13.11 (s, 1H), 8.45 (s, 1H), 7.79 (d, J=1.5 Hz, 1H), 7.66 (s, 1H), 7.63 (dd, J=7.8, 1.5 Hz, 1H), 7.45-7.35 (m, 2H), 7.10 (d, J=2.0 Hz, 1H), 7.02 (dd, J=8.9, 2.1 Hz, 1H), 6.00 (d, J=8.8 Hz, 1H), 5.31 (q, J=8.2 Hz, 1H), 2.49-2.42 (m, 1H), 1.83 (dd, J=12.5, 8.4 Hz, 1H), 1.40 (s, 3H), 1.30 (s, 3H). MS-ESI (m/z) calc'd for C₂₂H₂₀N₅O [M+H]⁺: 370.2. Found 370.2. A later eluting fraction was also isolated to afford 3,3-dimethyl-1-{[3-(1,3-oxazol-5-yl)-1H-indazol-5-yl]amino}-2,3-dihydro-1H-indene-5-carbonitrile, enantiomer 2 (17.2 mg, 24%) as a white solid. ¹H NMR (400 MHz, DMSO-d₆) δ 13.12 (s, 1H), 8.45 (s, 1H), 7.79 (d, J=1.5 Hz, 1H), 7.65 (s, 1H), 7.63 (dd, J=7.8, 1.5 Hz, 1H), 7.45-7.34 (m, 2H), 7.10 (d, J=2.0 Hz, 1H), 7.02 (dd, J=9.0, 2.1 Hz, 1H), 5.99 (d, J=8.7 Hz, 1H), 5.31 (q, J=8.2 Hz, 1H), 2.48-2.42 (m, 1H), 1.83 (dd, J=12.5, 8.4 Hz, 1H), 1.40 (s, 3H), 1.30 (s, 3H). MS-ESI (m/z) calc'd for C₂₂H₂₀N₅O [M+H]⁺: 370.2. Found 370.2.

### Example 106: 2-Methyl-8-((3-(oxazol-5-yl)-1H-indazol-5-yl)amino)-5,6,7,8-tetrahydroquinoline-3-carbonitrile, enantiomer 1 and 2

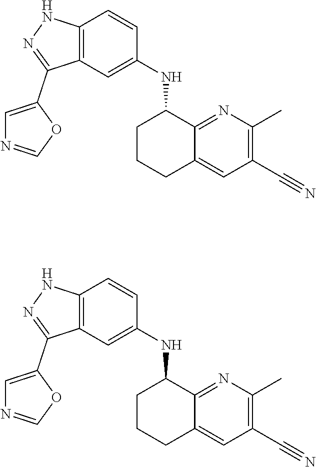 [SMILES: Cc1nc2c(cc1C#N)CCC[C@@H]2Nc1ccc2[nH]nc(-c3cnco3)c2c1 ; Cc1nc2c(cc1C#N)CCC[C@H]2Nc1ccc2[nH]nc(-c3cnco3)c2c1]

### Step 1: 2-Chloro-3-cyano-5,6,7,8-tetrahydroquinoline 1-oxide

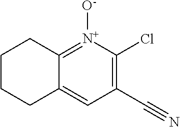 [SMILES: N#Cc1cc2c([n+]([O-])c1Cl)CCCC2]

To a solution of 2-chloro-5,6,7,8-tetrahydroquinoline-3-carbonitrile (2.0 g, 10.38 mmol) in trifluoroacetic acid (50 mL) was added hydrogen peroxide (3.18 mL, 31.15 mmol) and the mixture was stirred at 75° C. for 15 hrs. Water was added and the solution was neutralized by addition of solid K₂CO₃, then extracted with DCM (3×) and the combined organic layers were passed through a phase separator and evaporated to afford the title compound (1.94 g, 90%) as a yellow solid. ¹H NMR (400 MHz, DMSO-d₆) δ 7.79 (s, 1H) 2.78 (dt, J=16.56, 6.24 Hz, 4H) 1.77-1.86 (m, 2H) 1.63-1.72 (m, 2H). MS-ESI (m/z) calc'd for C₁₀H₁₀ClN[N+][O−] [M+H]⁺: 209.0, Found 209.0.

### Step 2: 2-Chloro-8-hydroxy-5,6,7,8-tetrahydroquinoline-3-carbonitrile

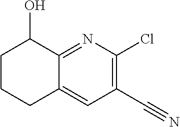 [SMILES: N#Cc1cc2c(nc1Cl)C(O)CCC2]

To a solution of 2-chloro-3-cyano-5,6,7,8-tetrahydroquinoline 1-oxide (1.94 g, 9.3 mmol) in DCM (50 mL) was added trifluoroacetic anhydride (3.88 mL, 27.89 mmol) dropwise and the mixture was stirred at 25° C. for 20 hrs. The solvent was evaporated and the residue was taken up in MeOH. K₂CO₃ was added till basic pH and the suspension was stirred at 25° C. for 1 hr. The solvent was evaporated, keeping the temperature under 40° C. The residue was taken up in water and extracted with DCM (3×). The combined organic layers were passed through a phase separator and evaporated to dryness. The material was purified by chromatography on a 50 g silica gel column using a 0-10% MeOH/DCM gradient eluent to afford the title compound (0.95 g, 49%) as a white solid. ¹H NMR (400 MHz, DMSO-d₆) δ 8.26 (s, 1H) 5.63 (d, J=5.28 Hz, 1H) 4.56 (q, J=4.84 Hz, 1H) 2.77-2.87 (m, 1H) 2.64-2.75 (m, 1H) 1.80-1.95 (m, 3H) 1.65-1.78 (m, 1H). MS-ESI (m/z) calc'd for C₁₀H₁₀ClN₂O [M+H]⁺: 209.0, Found 209.0.

### Step 3: 8-Hydroxy-2-methyl-5,6,7,8-tetrahydroquinoline-3-carbonitrile

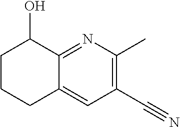

A solution of 2-chloro-8-hydroxy-5,6,7,8-tetrahydroquinoline-3-carbonitrile (150.0 mg, 0.720 mmol), K₂CO₃ (198.73 mg, 1.44 mmol) and 2,4,6-trimethyl-1,3,5,2,4,6-trioxatriborinane (0.2 mL, 1.44 mmol) in 1,4-dioxane (6 mL) and H₂O (3 mL) was degassed with N₂ for 15 min. Then Pd(PPh₃)₄ (166.16 mg, 0.140 mmol) was added and the mixture was stirred at 90° C. for 4 hrs. Due to incomplete reaction, the mixture was then heated to 100° C. in a microwave reactor for 15 min. Irradiation at 100° C. for 15 min was then repeated for an additional 2 cycles. The reaction mixture was partitioned between H₂O and DCM and the phases were separated. The aqueous layer was extracted with DCM (2×) and the combined organic phases were washed with water (1×), passed through a phase separator, and evaporated to dryness. The material was purified by silica gel chromatography on a 25 g column, using a 0-4% MeOH/DCM gradient eluent to afford the title compound (120 mg, 85%) as a brown solid, ¹H NMR (400 MHz, DMSO-d₆) δ 7.98 (s, 1H) 5.33 (d, J=4.62 Hz, 1H) 4.55 (q, J=4.47 Hz, 1H) 2.66-2.86 (m, 2H) 2.63 (s, 3H) 1.82-1.96 (m, 3H) 1.63-1.75 (m, 1H) MS-ESI (m/z) calc'd for C₁₁H₁₃N₂O [M+H]⁺: 189.1, Found 189.0.

### Step 4: N-(3-Cyano-2-methyl-5,6,7,8-tetrahydroquinolin-8-yl)-2-nitro-N-(3-(oxazol-5-yl)-1-((2-(trimethylsilyl)ethoxy)methyl)-1H-indazol-5-yl)benzenesulfonamide

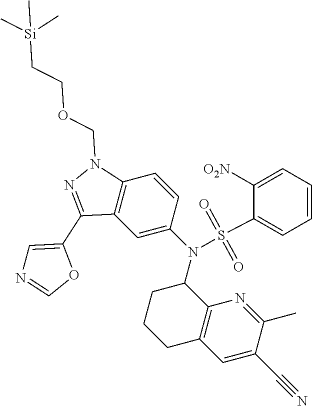 [SMILES: Cc1nc2c(cc1C#N)CCCC2N(c1ccc2c(c1)c(-c1cnco1)nn2COCC[Si](C)(C)C)S(=O)(=O)c1ccccc1[N+](=O)[O-]]

To a solution of 8-hydroxy-2-methyl-5,6,7,8-tetrahydroquinoline-3-carbonitrile

(220.0 mg, 0.61 mmol), 2-nitro-N-[3-(1,3-oxazol-5-yl)-1-(2-trimethylsilylethoxymethyl)indazol-5-yl]benzenesulfonamide (313.37 mg, 0.61 mmol) and triphenylphosphine (318.82 mg, 1.22 mmol) in THF (7 mL), was added dropwise diethyl azodicarboxylate (191.4 mL, 1.22 mmol) and the mixture was stirred at 25° C. for 1.5 hrs. The reaction mixture was partitioned between H₂O and EtOAc and the phases were separated. The aqueous layer was extracted with EtOAc (2×) and the combined organic phases were washed with water (1×), dried over anhydrous Na₂SO₄ and evaporated to dryness. The material was purified by silica gel chromatography on a 25 g column, using a 0-40% EtOAc/cyclohexane gradient eluent to afford the title compound (570 mg, 95%) as an orange solid. MS-ESI (m/z) calc'd for C₃₃H₃₆N₇O₆SSi [M+H]⁺: 686.2, Found 686.3.

### Step 5: 2-Methyl-8-((3-(oxazol-5-yl)-1-((2-(trimethylsilyl)ethoxy)methyl)-1H-indazol-5-yl)amino)-5,6,7,8-tetrahydroquinoline-3-carbonitrile

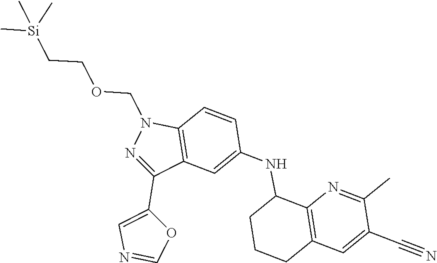 [SMILES: Cc1nc2c(cc1C#N)CCCC2Nc1ccc2c(c1)c(-c1cnco1)nn2COCC[Si](C)(C)C]

To a solution of N-(3-cyano-2-methyl-5,6,7,8-tetrahydroquinolin-8-yl)-2-nitro-N-(3-(oxazol-5-yl)-1-((2-(trimethylsilyl)ethoxy)methyl)-1H-indazol-5-yl)benzenesulfonamide (416.98 mg, 0.61 mmol) in DMF (7 mL) were added K₂CO₃ (336.13 mg, 2.43 mmol) and benzenethiol (0.19 mL, 1.82 mmol) and the mixture was stirred at r.t. for 2 hrs. The reaction mixture was partitioned between H₂O and EtOAc and the phases were separated. The aqueous layer was extracted with EtOAc (2×) and the combined organic phases were washed with water (1×), dried over anhydrous Na₂SO₄ and evaporated to dryness. The residue was purified by SCX, using a 5 g cartridge, washing with MeOH and then eluting the compound with a 2M solution of NH₃ in MeOH. The product containing fractions were combined and evaporated to dryness to afford the title compound (214 mg, 70%) as a yellow solid. ¹H NMR (400 MHz, DMSO-d₆) δ 8.52 (s, 1H) 8.07 (s, 1H) 7.75 (s, 1H) 7.56 (d, J=9.02 Hz, 1H) 7.17 (d, J=1.54 Hz, 1H) 7.08 (dd, J=9.13, 2.09 Hz, 1H) 6.02 (d, J=7.26 Hz, 1H) 5.71 (s, 2H) 4.75 (d, J=6.38 Hz, 1H) 3.48-3.63 (m, 2H) 2.71-2.94 (m, 2H) 2.60 (s, 3H) 1.87-2.08 (m, 3H) 1.75-1.85 (m, 1H) 0.77-0.86 (m, 2H) −0.11-−0.08 (m, 9H). MS-ESI (m/z) calc'd for C₂₇H₃₃N₆O₂Si [M+H]⁺: 501.2, Found 501.3.

### Step 6: 2-Methyl-8-((3-(oxazol-5-yl)-1H-indazol-5-yl)amino)-5,6,7,8-tetrahydroquinoline-3-carbonitrile

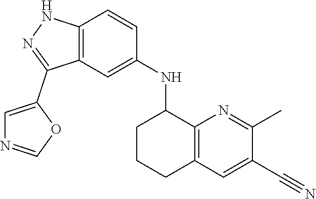 [SMILES: Cc1nc2c(cc1C#N)CCCC2Nc1ccc2[nH]nc(-c3cnco3)c2c1]

Prepared as described for 1-methoxy-5-{[3-(1,3-oxazol-5-yl)-1H-indazol-5-yl]amino}-5,6,7,8-tetrahydronaphthalene-2-carbonitrile using 2-methyl-8-{[3-(1,3-oxazol-5-yl)-1-{[2-(trimethylsilyl)ethoxy]methyl}-1H-indazol-5-yl]amino}-5,6,7,8-tetrahydroquinoline-3-carbonitrile in place of 1-methoxy-5-{[3-(1,3-oxazol-5-yl)-1-{[2-(trimethylsilyl)ethoxy]methyl}-1H-indazol-5-yl]amino}-5,6,7,8-tetrahydronaphthalene-2-carbonitrile, to afford the title compound (90.0 mg, 57%).

### Step 7: 2-Methyl-8-((3-(oxazol-5-yl)-1H-indazol-5-yl)amino)-5,6,7,8-tetrahydroquinoline-3-carbonitrile, enantiomer 1 and 2

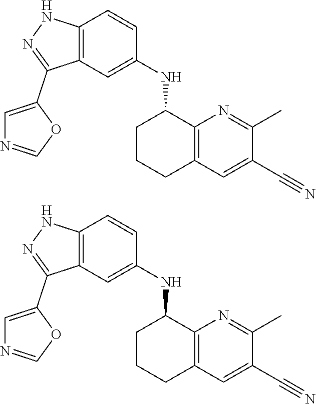 [SMILES: Cc1nc2c(cc1C#N)CCC[C@@H]2Nc1ccc2[nH]nc(-c3cnco3)c2c1 ; Cc1nc2c(cc1C#N)CCC[C@H]2Nc1ccc2[nH]nc(-c3cnco3)c2c1]

2-Methyl-8-((3-(oxazol-5-yl)-1H-indazol-5-yl)amino)-5,6,7,8-tetrahydroquinoline-3-carbonitrile was subjected to chiral separation using Method CR to afford 2-methyl-8-{[3-(1,3-oxazol-5-yl)-1H-indazol-5-yl]amino}-5,6,7,8-tetrahydroquinoline-3-carbonitrile, enantiomer 1 (24.6 mg, 15%) as a yellow solid. ¹H NMR (400 MHz, DMSO-d₆) δ 13.09 (br. s., 1H) 8.48 (s, 1H) 8.06 (s, 1H) 7.66 (s, 1H) 7.37 (d, J=8.80 Hz, 1H) 7.13 (d, J=1.10 Hz, 1H) 7.01 (dd, J=8.91, 2.09 Hz, 1H) 5.91 (d, J=7.04 Hz, 1H) 4.64-4.78 (m, 1H) 2.71-2.97 (m, 2H) 2.60 (s, 3H) 1.87-2.07 (m, 3H) 1.82 (d, J=3.30 Hz, 1H). MS-ESI (m/z) calc'd for C₂₁H₁₉N₆O [M+H]⁺: 371.1. Found 371.2. A later eluting fraction was also isolated to afford 2-methyl-8-{[3-(1,3-oxazol-5-yl)-1H-indazol-5-yl]amino}-5,6,7,8-tetrahydroquinoline-3-carbonitrile, enantiomer 2 (23.4 mg, 15%) as a yellow solid. ¹NMR (400 MHz, DMSO-d₆) δ 13.08 (br. s., 1H) 8.48 (s, 1H) 8.06 (s, 1H) 7.66 (s, 1H) 7.37 (d, J=8.80 Hz, 1H) 7.13 (d, J=1.32 Hz, 1H) 7.01 (dd, J=9.02, 1.98 Hz, 1H) 5.92 (d, J=7.48 Hz, 1H) 4.68-4.74 (m, 1H) 2.70-2.95 (m, 2H) 2.60 (s, 3H) 1.86-2.12 (m, 3H) 1.82 (d, J=3.74 Hz, 1H). MS-ESI (m/z) calc'd for C₂₁H₁₉N₆O [M+H]⁺: 371.1. Found 371.2.

### Example 107: 4-Methyl-7-((3-(oxazol-5-yl)-1H-indazol-5-yl)oxy)-6,7-dihydro-5H-cyclopenta[b]pyridine-3-carbonitrile, enantiomer 1 and 2

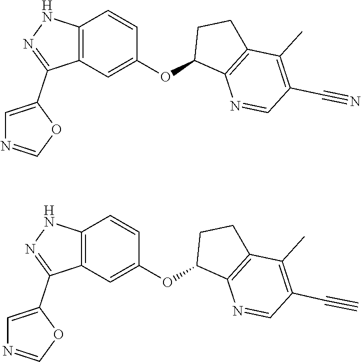 [SMILES: Cc1c(C#N)cnc2c1CC[C@@H]2Oc1ccc2[nH]nc(-c3cnco3)c2c1 ; C#Cc1cnc2c(c1C)CC[C@H]2Oc1ccc2[nH]nc(-c3cnco3)c2c1]

### Step 1: 4-Methyl-2-oxo-2,5,6,7-tetrahydro-1H-cyclopenta[b]pyridine-3-carbonitrile and 1-methyl-3-oxo-3,5,6,7-tetrahydro-2H-cyclopenta[c]pyridine-4-carbonitrile

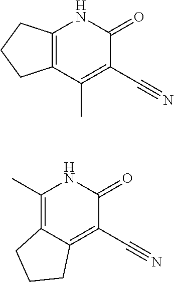

To a solution of 2-acetyl-1-cyclopentanone (2.52 g, 20.00 mmol) and 2-cyanoacetamide (1.68 g, 20 mmol) in EtOH (50 mL) was added piperidine (1.98 mL, 20 mmol) and the mixture was stirred at 75° C. overnight. After cooling the solid was filtered to obtain a mixture (˜1:1) of the title compounds (1.62 g, 46%) as a white solid. ¹H NMR (400 MHz, DMSO-d₆) δ 12.24 (s, 1H), 2.89 (t, J=7.6 Hz, 2H), 2.64 (t, J=7.4 Hz, 2H), 2.19 (s, 3H), 2.10-1.93 (m, 2H). MS-ESI (m/z) calc'd for C₁₀H₁₁N₂O [M+H]⁺: 175.1. Found 175.0. ¹H NMR (400 MHz, DMSO-d₆) δ 12.24 (s, 1H), 2.79 (t, J=7.7 Hz, 2H), 2.64 (t, J=7.4 Hz, 2H), 2.27 (s, 3H), 2.10-1.93 (m, 2H). MS-ESI (m/z) calc'd for C₁₀H₁₁N₂O [M+H]⁺: 175.1. Found 175.0.

### Step 2: 2-Chloro-4-methyl-2,5,6,7-tetrahydro-1H-cyclopenta[b]pyridine-3-carbonitrile and 3-Chloro-1-methyl-3,5,6,7-tetrahydro-2H-cyclopenta[c]pyridine-4-carbonitrile

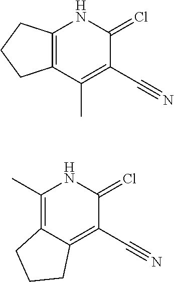

A suspension of 4-methyl-2-oxo-2,5,6,7-tetrahydro-1H-cyclopenta[b]pyridine-3-carbonitrile and 1-methyl-3-oxo-3,5,6,7-tetrahydro-2H-cyclopenta[c]pyridine-4-carbonitrile (1.62 g, 9.3 mmol) in POCl₃ (10.0 mL, 107.0 mmol) was heated at 100° C. overnight. The excess POCl₃ was evaporated and the remaining oil was taken up in water and stirred for 30 minutes. A solid formed that was collected by filtration and dried under vacuum to give a mixture (˜1:1) of the title compounds (1.79 g, 100%) as an off-white solid. ¹H NMR (400 MHz, DMSO-d₆) δ 3.08 (t, J=7.7 Hz, 2H), 2.90 (td, J=7.5, 3.7 Hz, 2H), 2.45 (s, 3H), 2.20-2.04 (m, 2H). MS-ESI (m/z) calc'd for C₁₀H₉N₂Cl [M+H]⁺: 193.0. Found 192.9. ¹H NMR (400 MHz, DMSO-d₆) δ 3.00 (dd, J=8.2, 7.4 Hz, 2H), 2.90 (td, J=7.5, 3.7 Hz, 2H), 2.43 (s, 3H), 2.20-2.04 (m, 2H). MS-ESI (m/z) calc'd for C₁₀H₉N₂Cl [M+H]⁺: 193.0. Found 193.0.

### Step 3: 4-Methyl-2,5,6,7-tetrahydro-1H-cyclopenta[b]pyridine-3-carbonitrile

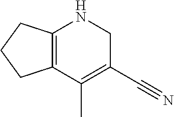

To a solution 2-chloro-4-methyl-2,5,6,7-tetrahydro-1H-cyclopenta[b]pyridine-3-carbonitrile and 3-chloro-1-methyl-3,5,6,7-tetrahydro-2H-cyclopenta[c]pyridine-4-carbonitrile (1.79 g, 9.30 mmol) in MeOH (46.5 mL) was added 10% palladium on carbon (0.99 g, 0.93 mmol). Then ammonium formate (1.76 g, 27.9 mmol) was added and the mixture was stirred at 60° C. for 1 hr. The catalyst was removed by filtration through Celite and the filtrate was evaporated to dryness. The residue was taken up in H₂O and extracted with DCM (3×). The combined organic layers were passed through a phase separator and evaporated to dryness. The material was purified by chromatography on a 28 g NH column using a 0-100% EtOAc/cyclohexane gradient eluent to afford the title compound (380 mg, 25%) as a white solid. ¹H NMR (400 MHz, DMSO-d₆) δ 8.65 (s, 1H), 2.99 (t, J=7.8 Hz, 2H), 2.96-2.88 (m, 3H), 2.39 (s, 4H), 2.08 (p, J=7.7 Hz, 3H). MS-ESI (m/z) calc'd for C₁₀H₁₁N₂ [M+H]⁺: 159.1. Found 158.9.

### Step 4: 3-Cyano-4-methyl-6,7-dihydro-5H-cyclopenta[b]pyridine 1-oxide

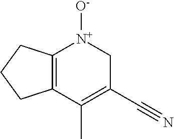

To a solution of 4-methyl-6,7-dihydro-5H-cyclopenta[b]pyridine-3-carbonitrile (360.0 mg, 2.28 mmol) in DCM (24.0 mL) was added MCPBA (561.0 mg, 2.28 mmol) and the mixture was stirred at 25° C. overnight. The mixture was diluted with DCM and quenched by addition of saturated aqueous Na₂S203. The organic layer washed with K₂CO₃ solution, passed through a phase separator and evaporated to afford the title compound (375 mg, 94%) as a beige solid. ¹H NMR (400 MHz, DMSO-d₆) δ 8.65 (s, 1H), 3.05-2.99 (m, 2H), 2.98 (t, J=7.7 Hz, 2H), 2.33 (s, 3H), 2.11 (p, J=7.8 Hz, 2H). MS-ESI (m/z) calc'd for C₁₀H₁₁N₂₀ [M+H]⁺: 175.1. Found 175.0.

### Step 5:7-Hydroxy-4-methyl-6,7-dihydro-5H-cyclopenta[b]pyridine-3-carbonitrile

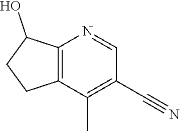

To a solution of 3-cyano-4-methyl-6,7-dihydro-5H-cyclopenta[b]pyridine 1-oxide (375.0 mg, 2.15 mmol) in DCM (11.0 mL) was added trifluoroacetic anhydride (0.9 mL, 6.46 mmol) and the mixture was stirred at 25° C. for 24 hrs. An aqueous solution of K₂CO₃ was then added and stirring was continued for an additional 6 hrs. The organic layer was passed through a phase separator and evaporated to afford the title compound (375 mg, 100%) as a dark solid. ¹H NMR (400 MHz, DMSO-d₆) δ 8.76 (s, 1H), 5.59 (d, J=5.7 Hz, 1H), 4.99 (dt, J=7.5, 5.8 Hz, 1H), 2.95 (ddd, J=16.6, 8.9, 4.6 Hz, 1H), 2.74 (ddd, J=16.1, 8.4, 6.9 Hz, 1H), 2.41 (s, 3H), 2.46-2.33 (m, 1H), 1.84 (dddd, J=12.9, 8.9, 6.7, 5.9 Hz, 1H). MS-ESI (m/z) calc'd for C₁₀H₁₁N₂O [M+H]⁺: 175.1. Found 175.0.

### Step 6: 4-Methyl-7-((3-(oxazol-5-yl)-1-((2-(trimethylsilyl)ethoxy)methyl)-1H-indazol-5-yl)oxy)-6,7-dihydro-5H-cyclopenta[b]pyridine-3-carbonitrile

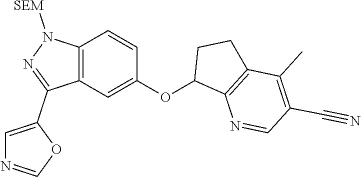 [SMILES: Cc1c(C#N)cnc2c1CCC2Oc1ccc2c(c1)c(-c1cnco1)nn2COCC[Si](C)(C)C]

To a solution of 3-(1,3-oxazol-5-yl)-1-(2-trimethylsilylethoxymethyl)indazol-5-ol (165.7 mg, 0.50 mmol), 7-hydroxy-4-methyl-6,7-dihydro-5H-cyclopenta[b]pyridine-3-carbonitrile (87.1 mg, 0.50 mmol) and triphenylphosphine (131.1 mg, 0.50 mmol) in DCM (5.0 mL) was added diethyl azodicarboxylate (78.7 uL, 0.50 mmol) and the mixture was stirred at 25° C. overnight. The solvent was evaporated, and the residue was passed through an SCX (5 g) cartridge to afford the title compound (100 mg, 41%) as a black solid which was used in the next step without further purification. MS-ESI (m/z) calc'd for C₂₆H₃₀N₅O₃Si [M+H]⁺: 488.2. Found 488.3.

### Step 7: 4-Methyl-7-((3-(oxazol-5-yl)-1H-indazol-5-yl)oxy)-6,7-dihydro-5H-cyclopenta[b]pyridine-3-carbonitrile

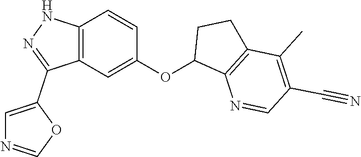 [SMILES: Cc1c(C#N)cnc2c1CCC2Oc1ccc2[nH]nc(-c3cnco3)c2c1]

Prepared as described for 1-methoxy-5-{[3-(1,3-oxazol-5-yl)-1H-indazol-5-yl]amino}-5,6,7,8-tetrahydronaphthalene-2-carbonitrile using 4-methyl-7-((3-(oxazol-5-yl)-1-((2-(trimethylsilyl)ethoxy)methyl)-1H-indazol-5-yl)oxy)-6,7-dihydro-5H-cyclopenta[b]pyridine-3-carbonitrile in place of 1-methoxy-5-{[3-(1,3-oxazol-5-yl)-1-{[2-(trimethylsilyl)ethoxy]methyl}-1H-indazol-5-yl]amino}-5,6,7,8-tetrahydronaphthalene-2-carbonitrile, to afford the title compound (20.0 mg, 83%).

### Step 8: 4-Methyl-7-((3-(oxazol-5-yl)-1H-indazol-5-yl)oxy)-6,7-dihydro-5H-cyclopenta[b]pyridine-3-carbonitrile, enantiomer 1 and 2

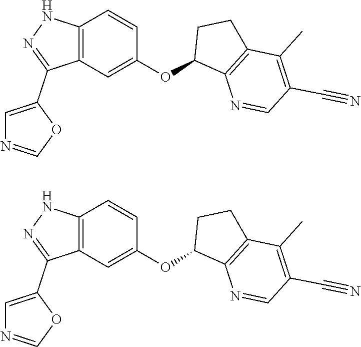 [SMILES: Cc1c(C#N)cnc2c1CC[C@@H]2Oc1ccc2[nH]nc(-c3cnco3)c2c1 ; Cc1c(C#N)cnc2c1CC[C@H]2Oc1ccc2[nH]nc(-c3cnco3)c2c1]

4-Methyl-7-((3-(oxazol-5-yl)-1H-indazol-5-yl)oxy)-6,7-dihydro-5H-cyclopenta[b]pyridine-3-carbonitrile was subjected to chiral separation using Method CS to afford 4-methyl-7-[[3-(1,3-oxazol-5-yl)-1H-indazol-5-yl]oxy]-6,7-dihydro-5H-cyclopenta[b]pyridine-3-carbonitrile, enantiomer 1 (3.0 mg, 12%) was obtained as a yellow solid. ¹H NMR (400 MHz, acetone-d₆) δ 12.49 (s, 1H), 8.75 (s, 1H), 8.27 (s, 1H), 7.84 (d, J=2.3 Hz, 1H), 7.66 (s, 1H), 7.59 (dd, J=9.0, 0.7 Hz, 1H), 7.23 (dd, J=9.0, 2.3 Hz, 1H), 5.93 (dd, J=7.1, 4.0 Hz, 1H), 3.21 (ddd, J=16.5, 8.7, 5.9 Hz, 1H), 3.05 (ddd, J=16.8, 8.8, 4.9 Hz, 1H), 2.85-2.74 (m, 1H), 2.55 (s, 3H), 2.36 (dddd, J=13.8, 8.8, 4.9, 4.0 Hz, 1H). MS-ESI (m/z) calc'd for C₂₀H₁₆N₅O₂ [M+H]⁺: 358.1. Found 358.1. A later eluting fraction was also isolated to afford 4-methyl-7-[[3-(1,3-oxazol-5-yl)-1H-indazol-5-yl]oxy]-6,7-dihydro-5H-cyclopenta[b]pyridine-3-carbonitrile, enantiomer 2 (1.2 mg, 5%) as a yellow solid. ¹H NMR (400 MHz, acetone-d₆) δ 12.49 (s, 1H), 8.75 (s, 1H), 8.27 (s, 1H), 7.84 (d, J=2.3 Hz, 1H), 7.66 (s, 1H), 7.59 (dd, J=9.0, 0.7 Hz, 1H), 7.23 (dd, J=9.0, 2.3 Hz, 1H), 5.93 (dd, J=7.1, 4.0 Hz, 1H), 3.21 (ddd, J=16.5, 8.7, 5.9 Hz, 1H), 3.05 (ddd, J=16.8, 8.8, 4.9 Hz, 1H), 2.85-2.74 (m, 1H), 2.55 (s, 3H), 2.36 (dddd, J=13.8, 8.8, 4.9, 4.0 Hz, 1H). MS-ESI (m/z) calc'd for C₂₀H₁₆N₅O₂ [M+H]⁺: 358.1. Found 358.1.

### Example 108: 4-Methyl-7-((3-(oxazol-yl)-1H-indazol-5-yl)amino)-6,7-dihydro-5H-cyclopenta[b]pyridine-3-carbonitrile

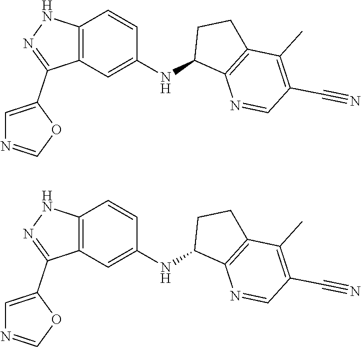 [SMILES: Cc1c(C#N)cnc2c1CC[C@@H]2Nc1ccc2[nH]nc(-c3cnco3)c2c1 ; Cc1c(C#N)cnc2c1CC[C@H]2Nc1ccc2[nH]nc(-c3cnco3)c2c1]

### Step 1: N-(3-Cyano-4-methyl-6,7-dihydro-5H-cyclopenta[b]pyridin-7-yl)-2-nitro-N-(3-(oxazol-5-yl)-1-((2-(trimethylsilyl)ethoxy)methyl)-1H-indazol-5-yl)benzenesulfonamide

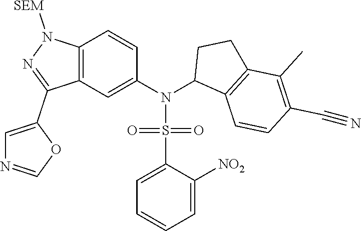 [SMILES: Cc1c(C#N)ccc2c1CCC2N(c1ccc2c(c1)c(-c1cnco1)nn2COCC[Si](C)(C)C)S(=O)(=O)c1ccccc1[N+](=O)[O-]]

To a solution of 7-hydroxy-4-methyl-6,7-dihydro-5H-cyclopenta[b]pyridine-3-carbonitrile (87.1 mg, 0.50 mmol) and 2-nitro-N-[3-(1,3-oxazol-5-yl)-1-(2-trimethylsilylethoxymethyl)indazol-5-yl]benzenesulfonamide (274.2 mg, 0.50 mmol) in THF (5.0 mL), were added triphenylphosphine (131.1 mg, 0.50 mmol) and diethyl azodicarboxylate (78.7 uL, 0.50 mmol). The mixture was stirred at 25° C. for 24 hrs. The solvent was evaporated to dryness and the residue was purified by chromatography on a 11 g NH column using a 0-100% EtOAc/cyclohexane gradient eluent afford the title compound (336 mg, 100%) as a beige solid. ¹H NMR (400 MHz, DMSO-d₆) δ 9.12-6.90 (m, 10H), 6.20-5.91 (m, 1H), 5.75 (d, J=8.4 Hz, 2H), 3.66-3.45 (m, 2H), 2.80-2.60 (m, 2H), 2.45-2.34 (m, 1H), 2.21 (d, J=4.2 Hz, 3H), 2.11-1.99 (m, 1H), 0.87-0.66 (m, 2H), −0.18 (s, 9H). MS-ESI (m/z) calc'd for C₃₂H₃₄N₇O₆SiS [M+H]⁺: 672.2. Found 672.1.

### Step 2: 4-Methyl-7-((3-(oxazol-5-yl)-1-((2-(trimethylsilyl)ethoxy)methyl)-1H-indazol-5-yl)amino)-6,7-dihydro-5H-cyclopenta[b]pyridine-3-carbonitrile

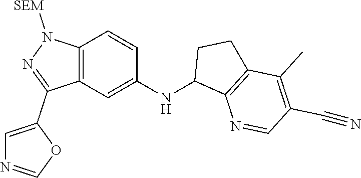 [SMILES: Cc1c(C#N)cnc2c1CCC2Nc1ccc2c(c1)c(-c1cnco1)nn2COCC[Si](C)(C)C]

To a solution of N-(3-cyano-4-methyl-6,7-dihydro-5H-cyclopenta[b]pyridin-7-yl)-2-nitro-N-(3-(oxazol-5-yl)-1-((2-(trimethylsilyl)ethoxy)methyl)-1H-indazol-5-yl)benzenesulfonamide (335.9 mg, 0.50 mmol) in DMF (5.0 mL) were added K₂CO₃ (276.4 mg, 2.00 mmol) and benzenethiol (153.4 uL, 1.50 mmol) and the mixture was stirred at 25° C. for 1 hr. Water was added and the mixture was extracted with EtOAc (3×). The combined organic layers were evaporated to give a yellow oil which was passed through an SCX cartridge (10 g) to afford the title compound (224 mg, 92%) as a dark oil. ¹H NMR (400 MHz, DMSO-d₆) δ 8.90-6.70 (m, 6H), 6.08 (d, J=7.2 Hz, 1H), 5.71 (s, 2H), 5.12 (p, J=7.5 Hz, 1H), 3.67-3.44 (m, 2H), 3.15-2.98 (m, 1H), 2.89 (s, 1H), 2.73 (d, J=0.7 Hz, 1H), 2.47 (s, 3H), 2.06-1.82 (m, 1H), 0.95-0.72 (m, 2H), 0.02-−0.20 (m, 9H). MS-ESI (m/z) calc'd for C₂₆H₃₁N₆O₂Si [M+H]⁺: 487.2. Found 487.2.

### Step 3: 4-Methyl-7-((3-(oxazol-5-yl)-1H-indazol-5-yl)amino)-6,7-dihydro-5H-cyclopenta[b]pyridine-3-carbonitrile

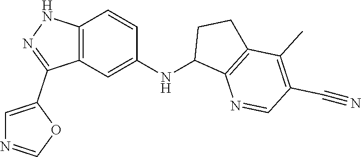 [SMILES: Cc1c(C#N)cnc2c1CCC2Nc1ccc2[nH]nc(-c3cnco3)c2c1]

Prepared as described for 1-methoxy-5-{[3-(1,3-oxazol-5-yl)-1H-indazol-5-yl]amino}-5,6,7,8-tetrahydronaphthalene-2-carbonitrile using 4-methyl-7-((3-(oxazol-5-yl)-1-((2-(trimethylsilyl)ethoxy)methyl)-1H-indazol-5-yl)amino)-6,7-dihydro-5H-cyclopenta[b]pyridine-3-carbonitrile in place of 1-methoxy-5-{[3-(1,3-oxazol-5-yl)-1-{[2-(trimethylsilyl)ethoxy]methyl}-1H-indazol-5-yl]amino}-5,6,7,8-tetrahydronaphthalene-2-carbonitrile, to afford the title compound (45 mg, 27%).

### Step 4: 4-Methyl-7-((3-(oxazol-yl)-1H-indazol-5-yl)amino)-6,7-dihydro-5H-cyclopenta[b]pyridine-3-carbonitrile, enantiomer 1 and 2

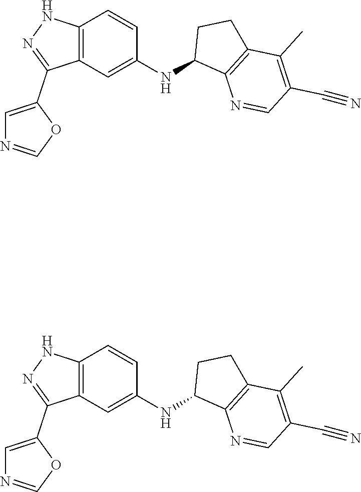 [SMILES: Cc1c(C#N)cnc2c1CC[C@@H]2Nc1ccc2[nH]nc(-c3cnco3)c2c1 ; Cc1c(C#N)cnc2c1CC[C@H]2Nc1ccc2[nH]nc(-c3cnco3)c2c1]

4-Methyl-7-((3-(oxazol-5-yl)-1H-indazol-5-yl)amino)-6,7-dihydro-5H-cyclopenta[b]pyridine-3-carbonitrile was subjected to chiral separation using Method CT to afford 4-methyl-7-((3-(oxazol-yl)-1H-indazol-5-yl)amino)-6,7-dihydro-5H-cyclopenta[b]pyridine-3-carbonitrile, enantiomer 1 (15.8 mg, 10%) was obtained as an off-white solid. ¹H NMR (400 MHz, DMSO-d₆) δ 13.09 (s, 1H), 8.75 (s, 1H), 8.46 (s, 1H), 7.63 (s, 1H), 7.37 (d, J=8.9 Hz, 1H), 7.14 (d, J=2.0 Hz, 1H), 7.02 (dd, J=9.0, 2.1 Hz, 1H), 5.96 (d, J=7.1 Hz, 1H), 5.09 (q, J=7.3 Hz, 1H), 3.05 (ddd, J=16.4, 8.9, 3.8 Hz, 1H), 2.89 (dt, J=16.3, 8.0 Hz, 1H), 2.70 (ddt, J=16.1, 8.0, 3.9 Hz, 1H), 2.47 (s, 3H), 2.01-1.86 (m, 1H). MS-ESI (m/z) calc'd for C₂₀H₁₇N₆O [M+H]⁺: 357.1. Found 357.2. A later eluting fraction was also isolated to afford 4-methyl-7-((3-(oxazol-yl)-1H-indazol-5-yl)amino)-6,7-dihydro-5H-cyclopenta[b]pyridine-3-carbonitrile, enantiomer 2 (13.5 mg, 8%) as an off-white solid. ¹H NMR (400 MHz, DMSO-d₆) δ 13.09 (s, 1H), 8.75 (s, 1H), 8.46 (s, 1H), 7.63 (s, 1H), 7.37 (d, J=9.0 Hz, 1H), 7.14 (s, 1H), 7.02 (dd, J=9.0, 2.1 Hz, 1H), 5.96 (d, J=7.0 Hz, 1H), 5.09 (q, J=7.3 Hz, 1H), 3.07-3.00 (m, 1H), 2.89 (dt, J=16.4, 8.0 Hz, 1H), 2.74-2.66 (m, 1H), 2.47 (s, 3H), 2.02-1.86 (m, 1H). MS-ESI (m/z) calc'd for C₂₀H₁₇N₆O [M+H]⁺: 357.1. Found 357.2.

### Example 109: 7-((3-(Oxazol-5-yl)-1H-indazol-5-yl)amino)-6,7-dihydro-5H-cyclopenta[b]pyridine-3-carbonitrile, enantiomer 1 and 2

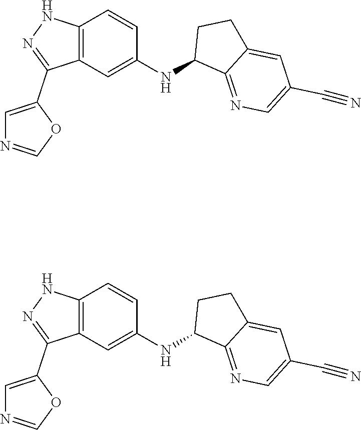 [SMILES: N#Cc1cnc2c(c1)CC[C@@H]2Nc1ccc2[nH]nc(-c3cnco3)c2c1 ; N#Cc1cnc2c(c1)CC[C@H]2Nc1ccc2[nH]nc(-c3cnco3)c2c1]

### Step 1: N-(3-Bromo-6,7-dihydro-5H-cyclopenta[b]pyridin-7-yl)-2-nitro-N-(3-(oxazol-5-yl)-1-((2-(trimethylsilyl)ethoxy)methyl)-1H-indazol-5-yl)benzenesulfonamide

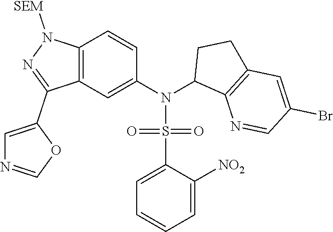 [SMILES: C[Si](C)(C)CCOCn1nc(-c2cnco2)c2cc(N(C3CCc4cc(Br)cnc43)S(=O)(=O)c3ccccc3[N+](=O)[O-])ccc21]

To a solution of 3-bromo-6,7-dihydro-5H-cyclopenta[b]pyridin-7-ol (100.0 mg, 0.47 mmol), 2-nitro-N-(3-(oxazol-5-yl)-1-((2-(trimethylsilyl)ethoxy)methyl)-1H-indazol-5-yl)benzenesulfonamide (256.3 mg, 0.47 mmol) and triphenylphosphine (245.1 mg, 0.93 mmol) in THF (4.7 mL), was added dropwise diethyl azodicarboxylate (147 μL, 0.93 mmol) and the mixture was stirred at 25° C. for 15 hrs. The solvent was evaporated under reduced pressure and the material was purified by chromatography on a 10 g NH-silica gel column, using a 0-50% EtOAc/cyclohexane gradient eluent. Selected fractions were combined and evaporated to dryness. The residue was further purified by additional chromatography on a 10 g NH-silica gel column, using a 0-50% EtOAc/cyclohexane gradient eluent to afford the title compound (320 mg, 96%) as an orange solid. MS-ESI (m/z) calc'd for C₃₀H₃₂BrN₆O₆SSi [M+H]⁺: 711.1; 713.1. Found 711.1; 713.1.

### Step 2: N-(3-Cyano-6,7-dihydro-5H-cyclopenta[b]pyridin-7-yl)-2-nitro-N-(3-(oxazol-5-yl)-1-((2-(trimethylsilyl)ethoxy)methyl)-1H-indazol-5-yl)benzenesulfonamide

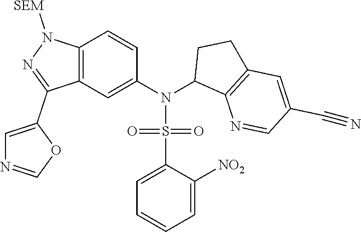 [SMILES: C[Si](C)(C)CCOCn1nc(-c2cnco2)c2cc(N(C3CCc4cc(C#N)cnc43)S(=O)(=O)c3ccccc3[N+](=O)[O-])ccc21]

A 0.1 N aqueous solution of potassium hexacyanoferrate (II) (3.93 mL, 0.39 mmol), N-(3-bromo-6,7-dihydro-5H-cyclopenta[b]pyridin-7-yl)-2-nitro-N-(3-(oxazol-5-yl)-1-((2-(trimethylsilyl)ethoxy)methyl)-1H-indazol-5-yl)benzenesulfonamide (280.0 mg, 0.39 mmol) and KOAc (38.6 mg, 0.39 mmol) were dissolved in a mixture of 1,4-dioxane (3.78 mL) and H₂O (0.377 mL) in a sealed microwave vial. The mixture was degassed with N₂ for 15 minutes. Then XPhos (15.0 mg, 0.03 mmol) and XPhos Pd G3 (26.6 mg, 0.03 mmol) were added and the mixture was left stirring at 100° C. for 4 hrs. The reaction mixture was partitioned between H₂O and EtOAc and the phases were separated. The aqueous layer was extracted with EtOAc (3×) and the combined organic phases were washed with brine (2×), dried over anhydrous Na₂SO₄, filtered and evaporated to dryness. The material was purified by chromatography on a 25 g silica gel column using a 0-50% EtOAc/cyclohexane gradient eluent to afford the title compound (124.4 mg, 48%) as an orange solid. ¹H NMR (400 MHz, DMSO-d₆) δ 9.05 (d, J=2.1 Hz, 1H), 8.59 (s, 1H), 8.02 (dd, J=7.9, 1.3 Hz, 2H), 7.99-7.91 (m, 2H), 7.80 (td, J=7.6, 1.4 Hz, 1H), 7.68 (d, J=9.0 Hz, 1H), 7.57 (s, 1H), 7.49 (d, J=1.9 Hz, 1H), 7.00 (dd, J=8.9, 2.0 Hz, 1H), 6.06 (dd, J=8.5, 7.1 Hz, 1H), 5.74 (s, 2H), 3.50 (t, J=7.9 Hz, 2H), 2.78 (dt, J=15.9, 7.6 Hz, 1H), 2.68 (ddd, J=13.2, 8.6, 4.3 Hz, 1H), 2.41-2.30 (m, 1H), 2.17-2.03 (m, 1H), 0.76 (td, J=7.5, 2.0 Hz, 2H), −0.18 (s, 9H). MS-ESI (m/z) calc'd for C₃₁H₃₂N₇O₆SSi [M+H]⁺: 658.2. Found 658.3.

### Step 3: 7-((3-(Oxazol-5-yl)-1-((2-(trimethylsilyl)ethoxy)methyl)-1H-indazol-5-yl)amino)-6,7-dihydro-5H-cyclopenta[b]pyridine-3-carbonitrile

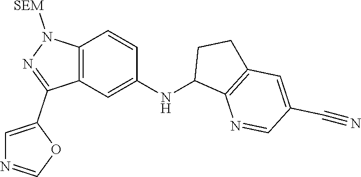 [SMILES: C[Si](C)(C)CCOCn1nc(-c2cnco2)c2cc(NC3CCc4cc(C#N)cnc43)ccc21]

To a solution of N-(3-cyano-6,7-dihydro-5H-cyclopenta[b]pyridin-7-yl)-2-nitro-N-(3-(oxazol-5-yl)-1-((2-(trimethylsilyl)ethoxy)methyl)-1H-indazol-5-yl)benzenesulfonamide (124.4 mg, 0.15 mmol) in DMF (1.5 mL) was added K₂CO₃ (84.7 mg, 0.16 mmol) and benzenethiol (0.047 mL, 0.46 mmol), the mixture was stirred at r.t. for 2 hrs. The reaction mixture was partitioned between H₂O and EtOAc and the phases were separated. The aqueous layer was extracted with EtOAc (2×) and the combined organic phases were washed with brine, dried over anhydrous Na₂SO₄, and evaporated to dryness. The residue was purified by SCX using a 5 g cartridge, washed with MeOH and then eluting the compound with a 2M solution of NH₃ in MeOH. Selected fractions were combined and evaporated to dryness to afford the title compound (72 mg, 99%) as a yellow solid. ¹H NMR (400 MHz, MeOD) δ 8.77-8.67 (m, 1H), 8.35 (s, 1H), 8.08 (p, J=1.1 Hz, 1H), 7.64 (s, 1H), 7.53 (d, J=9.0 Hz, 1H), 7.28 (d, J=2.1 Hz, 1H), 7.14 (dd, J=9.0, 2.2 Hz, 1H), 5.74 (s, 2H), 5.12 (t, J=7.6 Hz, 1H), 3.64-3.56 (m, 2H), 3.14 (ddd, J=12.7, 8.8, 4.5 Hz, 1H), 3.05 (dt, J=16.7, 8.1 Hz, 1H), 2.83 (ddt, J=16.2, 7.9, 3.8 Hz, 1H), 2.08 (dq, J=12.9, 8.3 Hz, 1H), 0.92-0.81 (m, 2H), −0.08 (s, 9H). MS-ESI (m/z) calc'd for C₂₅H₂₉N₆O₂Si [M+H]⁺: 473.2. Found 473.3.

### Step 4: 7-((3-(Oxazol-5-yl)-1H-indazol-5-yl)amino)-6,7-dihydro-5H-cyclopenta[b]pyridine-3-carbonitrile

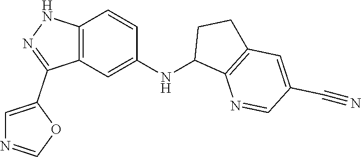 [SMILES: N#Cc1cnc2c(c1)CCC2Nc1ccc2[nH]nc(-c3cnco3)c2c1]

Prepared as described for 1-methoxy-5-{[3-(1,3-oxazol-5-yl)-1H-indazol-5-yl]amino}-5,6,7,8-tetrahydronaphthalene-2-carbonitrile using 7-((3-(oxazol-5-yl)-1-((2-(trimethylsilyl)ethoxy)methyl)-1H-indazol-5-yl)amino)-6,7-dihydro-5H-cyclopenta[b]pyridine-3-carbonitrile in place of 1-methoxy-5-{[3-(1,3-oxazol-5-yl)-1-{[2-(trimethylsilyl)ethoxy]methyl}-1H-indazol-5-yl]amino}-5,6,7,8-tetrahydronaphthalene-2-carbonitrile, to afford the title compound (50 mg, 88%).

### Step 5: 7-((3-(Oxazol-5-yl)-1H-indazol-5-yl)amino)-6,7-dihydro-5H-cyclopenta[b]pyridine-3-carbonitrile, enantiomer 1 and 2

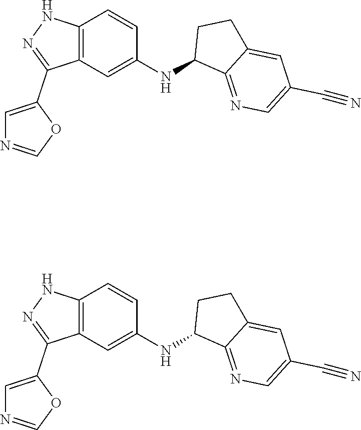 [SMILES: N#Cc1cnc2c(c1)CC[C@@H]2Nc1ccc2[nH]nc(-c3cnco3)c2c1 ; N#Cc1cnc2c(c1)CC[C@H]2Nc1ccc2[nH]nc(-c3cnco3)c2c1]

7-((3-(Oxazol-5-yl)-1H-indazol-5-yl)amino)-6,7-dihydro-5H-cyclopenta[b]pyridine-3-carbonitrile was purified by chiral separation using Method CU to afford 7-((3-(oxazol-5-yl)-1H-indazol-5-yl)amino)-6,7-dihydro-5H-cyclopenta[b]pyridine-3-carbonitrile, enantiomer 1 (11 mg, 21%) as a beige solid. ¹H NMR (400 MHz, DMSO-d₆) δ 13.09 (s, 1H), 8.82 (d, J=1.9 Hz, 1H), 8.46 (s, 1H), 8.30-8.13 (m, 1H), 7.64 (s, 1H), 7.37 (d, J=8.9 Hz, 1H), 7.15 (d, J=2.0 Hz, 1H), 7.02 (dd, J=9.0, 2.1 Hz, 1H), 5.99 (d, J=7.3 Hz, 1H), 5.12 (q, J=7.5 Hz, 1H), 3.06 (ddd, J=12.4, 8.6, 4.4 Hz, 1H), 2.95 (dt, J=16.4, 8.1 Hz, 1H), 2.76-2.62 (m, 1H), 1.95 (dq, J=12.5, 8.2 Hz, 1H). MS-ESI (m/z) calc'd for C₁₉H₁₅N₆₀ [M+H]⁺: 343.1. Found 343.2. A later eluting fraction was also isolated to afford 7-((3-(oxazol-5-yl)-1H-indazol-5-yl)amino)-6,7-dihydro-5H-cyclopenta[b]pyridine-3-carbonitrile, enantiomer 2 (11.3 mg, 22%) as a beige solid. ¹H NMR (400 MHz, DMSO-d₆) δ 13.09 (s, 1H), 8.82 (d, J=1.9 Hz, 1H), 8.46 (s, 1H), 8.22 (d, J=1.9 Hz, 1H), 7.64 (s, 1H), 7.37 (d, J=8.9 Hz, 1H), 7.15 (d, J=2.0 Hz, 1H), 7.02 (dd, J=9.0, 2.1 Hz, 1H), 5.99 (d, J=7.2 Hz, 1H), 5.12 (q, J=7.4 Hz, 1H), 3.06 (ddd, J=16.6, 8.7, 3.8 Hz, 1H), 2.95 (dt, J=16.4, 8.1 Hz, 1H), 2.76-2.63 (m, 1H), 2.02-1.88 (m, 1H). MS-ESI (m/z) calc'd for C₁₉H₁₅N₆O [M+H]⁺: 343.1. Found 343.2.

### Example 110: 3-Methyl-5-((3-(oxazol-5-yl)-1H-indazol-5-yl)amino)-5,6,7,8-tetrahydronaphthalene-2-carbonitrile, enantiomer 1 and 2

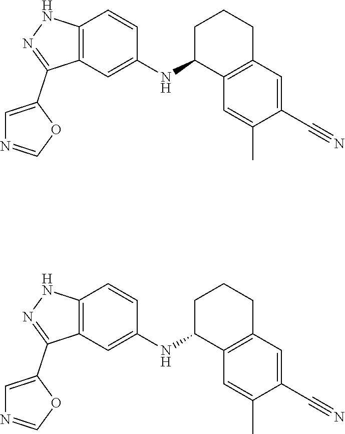 [SMILES: Cc1cc2c(cc1C#N)CCC[C@@H]2Nc1ccc2[nH]nc(-c3cnco3)c2c1 ; Cc1cc2c(cc1C#N)CCC[C@H]2Nc1ccc2[nH]nc(-c3cnco3)c2c1]

### Step 1: 3-Methyl-5-oxo-5,6,7,8-tetrahydronaphthalene-2-carbonitrile

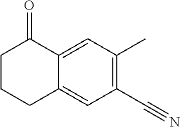

A 0.1 N aqueous solution of potassium hexacyanoferrate (II) (10.46 mL, 1.05 mmol), 6-bromo-7-methyl-1,2,3,4-tetrahydronaphtalen-1-one (250.0 mg, 1.05 mmol) and KOAc (102.61 mg, 1.05 mmol) were dissolved in a mixture of 1,4-dioxane (10 mL) and H₂O (1 mL) in a sealed microwave vial. The mixture was degassed with N₂ for 15 minutes. Then XPhos (18.5 mg, 0.04 mmol) and XPhos Pd G3 (32.8 mg, 0.04 mmol) were added and the mixture was left stirring at 100° C. for 1 hr. XPhos (37.0 mg, 0.08 mmol) and XPhos Pd G3 (65.6 mg, 0.04 mmol) were added again and the mixture was stirred at 100° C. for 3 hrs. The reaction mixture was partitioned between H₂O and EtOAc. The phases were separated and the aqueous layer was extracted with EtOAc (2×). The combined organic phases were washed with H₂O (1×), dried over anhydrous Na₂SO₄, and evaporated to dryness. The material was purified by chromatography on a 25 g silica gel column using a 0-15% EtOAc/cyclohexane gradient eluent to obtain a residue (316 mg) which was further purified by chromatography on a 28 g NH silica gel column, using a 0-20% EtOAc/cyclohexane gradient eluent to afford the title compound (100 mg, 52%) as a white solid. ¹H NMR (400 MHz, DMSO-d₆) δ 7.88 (s, 1H) 7.84 (s, 1H) 2.95 (t, J=6.05 Hz, 2H) 2.62-2.68 (m, 2H) 2.50 (s, 3H, peak under DMSO signal) 2.05 (quin, J=6.38 Hz, 2H). MS-ESI (m/z) calc'd for C₁₂H₁₂NO [M+H]⁺: 186.1, Found 186.1.

### Step 2: 5-Hydroxy-3-methyl-5,6,7,8-tetrahydronaphthalene-2-carbonitrile

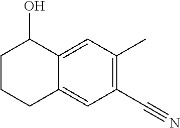

To a solution of 3-methyl-5-oxo-7,8-dihydro-6H-naphthalene-2-carbonitrile (100.0 mg, 0.52 mmol) in MeOH (5 mL) was added sodium borohydride (39.62 mg, 1.05 mmol) and the mixture was stirred at 25° C. for 30 minutes. The solvent was evaporated and the residue was taken up in H₂O and extracted with DCM (3×). The combined organic layers were passed through a phase separator and evaporated to afford the title compound (92 mg, 94%) as a white solid. ¹H NMR (400 MHz, DMSO-d₆) δ 7.47 (s, 2H) 5.35 (d, J=5.94 Hz, 1H) 4.50-4.62 (m, 1H) 2.59-2.80 (m, 2H) 2.43 (s, 3H) 1.80-1.99 (m, 2H) 1.58-1.73 (m, 2H) MS-ESI (m/z) calc'd for C₁₂H₁₄NO [M+H]⁺: 188.1, Found 188.1.

### Step 3: N-(6-Cyano-7-methyl-1,2,3,4-tetrahydronaphthalen-1-yl)-2-nitro-N-(3-(oxazol-5-yl)-1-((2-(trimethylsilyl)ethoxy)methyl)-1H-indazol-5-yl)benzenesulfonamide

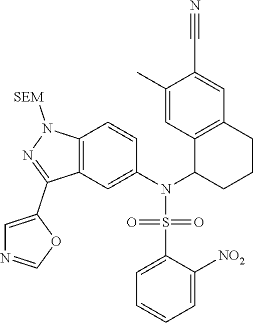 [SMILES: Cc1cc2c(cc1C#N)CCCC2N(c1ccc2c(c1)c(-c1cnco1)nn2COCC[Si](C)(C)C)S(=O)(=O)c1ccccc1[N+](=O)[O-]]

To a solution of 5-hydroxy-3-methyl-5,6,7,8-tetrahydronaphthalene-2-carbonitrile (90.0 mg, 0.480 mmol), 2-nitro-N-[3-(1,3-oxazol-5-yl)-1-(2-trimethylsilylethoxymethyl)indazol-5-yl]benzenesulfonamide (247.84 mg, 0.48 mmol) and triphenylphosphine (252.15 mg, 0.96 mmol) in THF (6 mL), was added dropwise diethyl azodicarboxylate (151.37 uL, 0.96 mmol) and the mixture was stirred at 25° C. for 18 hrs. The reaction mixture was partitioned between water and EtOAc, the phases were separated, and the aqueous layer was extracted with EtOAc (2×). The combined organic phases were washed with H₂O, dried over anhydrous Na₂SO₄, and evaporated to dryness. The material was purified by chromatography on a 25 g silica gel column using a 0-80% EtOAc/cyclohexane gradient eluent to afford the title compound (350 mg, 87%) as an orange solid. MS-ESI (m/z) calc'd for C₃₄H₃₇N₆O₆SSi [M+H]⁺: 685.2, Found 685.3.

### Step 4: 3-Methyl-5-((3-(oxazol-5-yl)-1-((2-(trimethylsilyl)ethoxy)methyl)-1H-indazol-5-yl)amino)-5,6,7,8-tetrahydronaphthalene-2-carbonitrile

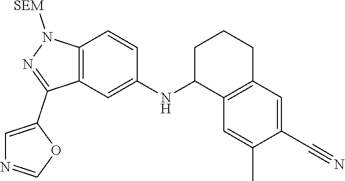 [SMILES: Cc1cc2c(cc1C#N)CCCC2Nc1ccc2c(c1)c(-c1cnco1)nn2COCC[Si](C)(C)C]

To a solution of N-(6-cyano-7-methyl-1,2,3,4-tetrahydronaphthalen-1-yl)-2-nitro-N-[3-(1,3-oxazol-5-yl)-1-(2-trimethylsilylethoxymethyl)indazol-5-yl]benzenesulfonamide (450.0 mg, 0.47 mmol) in DMF (7 mL) was added K₂CO₃ (261.55 mg, 1.89 mmol) and benzenethiol (0.15 mL, 1.42 mmol), the mixture was stirred at 25° C. for 2 hrs. The reaction mixture was partitioned between H₂O and EtOAc and the phases were separated. The aqueous layer was extracted with EtOAc (2×) and the combined organic phases were washed with H₂O (1×), dried over anhydrous Na₂SO₄, and evaporated to dryness. The residue was purified by SCX using a 5 g cartridge, washing with MeOH and then eluting the compound with a 2M solution of NH₃ in MeOH. The product-containing fractions were combined, evaporated to dryness, and the residue was purified again by chromatography on a 10 g silica gel column using a 0-40% EtOAc/cyclohexane gradient eluent to afford the title compound (159 mg, 67%) as a light yellow solid. ¹H NMR (400 MHz, DMSO-d₆) δ 8.48-8.54 (m, 1H) 7.74 (s, 1H) 7.54-7.61 (m, 2H) 7.44 (s, 1H) 7.04-7.13 (m, 2H) 6.02 (d, J=9.24 Hz, 1H) 5.67-5.75 (m, 2H) 4.82 (m, J=7.04 Hz, 1H) 3.49-3.65 (m, 3H) 2.69-2.88 (m, 2H) 2.39 (s, 3H) 1.73-2.03 (m, 4H) 0.75-0.92 (m, 2H) −0.11-−0.08 (m, 9H). MS-ESI (m/z) calc'd for C₂₈H₃₄N₅O₂Si [M+H]⁺: 501.3, Found 501.3.

### Step 5: 3-Methyl-5-((3-(oxazol-5-yl)-1H-indazol-5-yl)amino)-5,6,7,8-tetrahydronaphthalene-2-carbonitrile

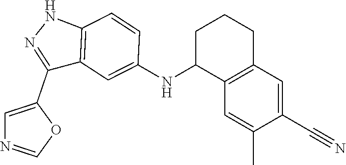 [SMILES: Cc1cc2c(cc1C#N)CCCC2Nc1ccc2[nH]nc(-c3cnco3)c2c1]

Prepared as described for 1-methoxy-5-{[3-(1,3-oxazol-5-yl)-1H-indazol-5-yl]amino}-5,6,7,8-tetrahydronaphthalene-2-carbonitrile using 3-methyl-5-{[3-(1,3-oxazol-5-yl)-1-{[2-(trimethylsilyl)ethoxy]methyl}-1H-indazol-5-yl]amino}-5,6,7,8-tetrahydronaphthalene-2-carbonitrile in place of 1-methoxy-5-{[3-(1,3-oxazol-5-yl)-1-{[2-(trimethylsilyl)ethoxy]methyl}-1H-indazol-5-yl]amino}-5,6,7,8-tetrahydronaphthalene-2-carbonitrile, to afford 3-methyl-5-{[3-(1,3-oxazol-5-yl)-1H-indazol-5-yl]amino}-5,6,7,8-tetrahydronaphthalene-2-carbonitrile (65 mg, 55%).

### Step 6: 3-Methyl-5-((3-(oxazol-5-yl)-1H-indazol-5-yl)amino)-5,6,7,8-tetrahydronaphthalene-2-carbonitrile, enantiomer 1 and 2

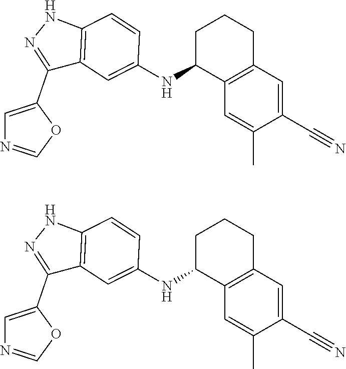 [SMILES: Cc1cc2c(cc1C#N)CCC[C@@H]2Nc1ccc2[nH]nc(-c3cnco3)c2c1 ; Cc1cc2c(cc1C#N)CCC[C@H]2Nc1ccc2[nH]nc(-c3cnco3)c2c1]

3-Methyl-5-((3-(oxazol-5-yl)-1H-indazol-5-yl)amino)-5,6,7,8-tetrahydronaphthalene-2-carbonitrile was subjected to chiral separation using Method CV to afford 3-methyl-5-((3-(oxazol-5-yl)-1H-indazol-5-yl)amino)-5,6,7,8-tetrahydronaphthalene-2-carbonitrile, enantiomer 1 (18.3 mg, 16%) as a yellow solid. ¹H NMR (400 MHz, DMSO-d₆) δ 13.09 (br. s., 1H) 8.46 (s, 1H) 7.66 (s, 1H) 7.57 (s, 1H) 7.45 (s, 1H) 7.38 (d, J=9.02 Hz, 1H) 6.97-7.08 (m, 2H) 5.91 (d, J=9.24 Hz, 1H) 4.73-4.83 (m, 1H) 2.69-2.88 (m, 2H) 2.40 (s, 3H) 1.73-2.03 (m, 4H). MS-ESI (m/z) calc'd for C₂₂H₂₀N₅O [M+H]⁺: 370.2. Found 370.2. A later eluting fraction was also isolated to afford 3-methyl-5-((3-(oxazol-5-yl)-1H-indazol-5-yl)amino)-5,6,7,8-tetrahydronaphthalene-2-carbonitrile, enantiomer 2 (17.2 mg, 15%) as a yellow solid. ¹H NMR (400 MHz, DMSO-d₆) δ 13.09 (br. s., 1H) 8.46 (s, 1H) 7.66 (s, 1H) 7.57 (s, 1H) 7.45 (s, 1H) 7.38 (d, J=9.02 Hz, 1H) 7.05 (s, 1H) 7.01 (dd, J=9.02, 1.98 Hz, 1H) 5.91 (d, J=9.02 Hz, 1H) 4.74-4.83 (m, 1H) 2.70-2.88 (m, 2H) 2.40 (s, 3H) 1.73-2.02 (m, 4H). MS-ESI (m/z) calc'd for C₂₂H₂₀N₅O [M+H]⁺: 370.2. Found 370.2.

### Example 111: 1-(Methyl(3-(oxazol-5-yl)-1H-indazol-5-yl)amino)-2,3-dihydro-1H-indene-5-carbonitrile, enantiomer 1 and 2

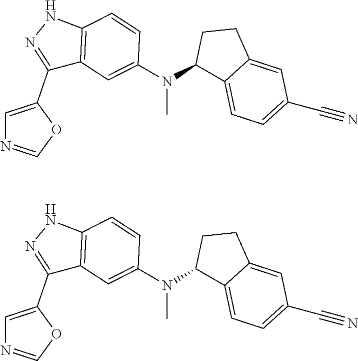 [SMILES: CN(c1ccc2[nH]nc(-c3cnco3)c2c1)[C@H]1CCc2cc(C#N)ccc21 ; CN(c1ccc2[nH]nc(-c3cnco3)c2c1)[C@@H]1CCc2cc(C#N)ccc21]

### Step 1: 1-((3-(Oxazol-5-yl)-1-((2-(trimethylsilyl)ethoxy)methyl)-1H-indazol-5-yl)amino)-2,3-dihydro-1H-indene-5-carbonitrile

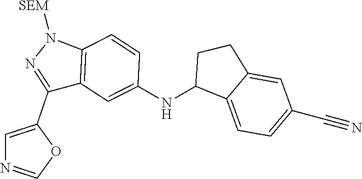 [SMILES: C[Si](C)(C)CCOCn1nc(-c2cnco2)c2cc(NC3CCc4cc(C#N)ccc43)ccc21]

To a stirred solution of 3-(1,3-oxazol-5-yl)-1-{[2-(trimethylsilyl)ethoxy]methyl}-1H-indazol-5-amine (504.6 mg, 1.53 mmol) and 1-oxo-2,3-dihydro-1H-indene-5-carbonitrile (200.0 mg, 1.27 mmol) in 1,4-dioxane (12.7 mL) was added 4-methylbenzenesulfonic acid hydrate (24.2 mg, 0.13 mmol) and the mixture was stirred at 100° C. for 1.5 hrs. The reaction was cooled to 40° C. and sodium triacetoxyborohydride (245.0 mg, 1.27 mmol) was added portionwise over 1.5 hrs. Then the mixture was stirred at 40° C. overnight and then partitioned between H₂O and EtOAc. The phases were separated and the organic layer was washed with saturated aqueous NH₄Cl (1×), dried over anhydrous Na₂SO₄, filtered and evaporated to dryness. The material was purified by chromatography on a 25 g silica gel column, using a 0-50% EtOAc/cyclohexane gradient eluent to afford the title compound (72 mg, 12%). ¹H NMR (400 MHz, CDCl₃) δ 8.02 (s, 1H), 7.60 (s, 1H), 7.56 (s, 1H), 7.54-7.47 (m, 3H), 7.19 (d, J=2.3 Hz, 1H), 6.98 (dd, J=9.0, 2.2 Hz, 1H), 5.76 (s, 2H), 5.28-5.08 (m, 1H), 3.98 (s, 1H), 3.66-3.57 (m, 2H), 3.18-3.07 (m, 1H), 3.07-2.95 (m, 1H), 2.83-2.71 (m, 1H), 2.02 (dq, J=12.6, 8.3 Hz, 1H), 0.97-0.87 (m, 2H), −0.03 (s, 9H). MS-ESI (m/z) calc'd for C₂₆H₃₀N₅O₂Si [M+H]⁺: 472.2. Found 472.2.

### Step 2: 1-(Methyl(3-(oxazol-5-yl)-1-((2-(trimethylsilyl)ethoxy)methyl)-1H-indazol-5-yl)amino)-2,3-dihydro-1H-indene-5-carbonitrile

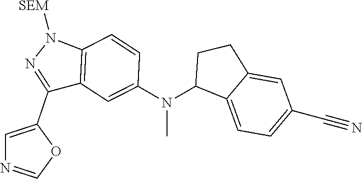 [SMILES: CN(c1ccc2c(c1)c(-c1cnco1)nn2COCC[Si](C)(C)C)C1CCc2cc(C#N)ccc21]

To a stirred solution of 1-((3-(oxazol-5-yl)-1-((2-(trimethylsilyl)ethoxy)methyl)-1H-indazol-5-yl)amino)-2,3-dihydro-1H-indene-5-carbonitrile (50.0 mg, 0.11 mmol) in acetonitrile (1 mL) were added Cs₂CO₃ (69.08 mg, 0.21 mmol) and iodomethane (33.0 uL, 0.53 mmol). The resulting mixture was heated at 80° C. for 2 hrs. Then the mixture was diluted with EtOAc and washed with saturated aqueous NaHCO₃. The phases were separated and the organic layer was dried over Na₂SO₄, filtered, and evaporated to dryness. The material was combined with material from a second 20 mg reaction and purified by chromatography on a 25 g silica gel column, using a 0-50% EtOAc/cyclohexane gradient eluent to afford the title compound (20 mg, 28%) as a yellow solid. MS-ESI (m/z) calc'd for C₂₇H₃₂N₅O₂Si [M+H]⁺: 486.2. Found 486.2.

### Step 3: 1-(Methyl(3-(oxazol-5-yl)-1H-indazol-5-yl)amino)-2,3-dihydro-1H-indene-5-carbonitrile

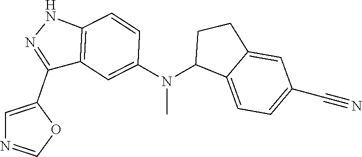 [SMILES: CN(c1ccc2[nH]nc(-c3cnco3)c2c1)C1CCc2cc(C#N)ccc21]

Prepared as described for 1-methoxy-5-{[3-(1,3-oxazol-5-yl)-1H-indazol-5-yl]amino}-5,6,7,8-tetrahydronaphthalene-2-carbonitrile using 1-(methyl(3-(oxazol-5-yl)-1-((2-(trimethylsilyl)ethoxy)methyl)-1H-indazol-5-yl)amino)-2,3-dihydro-1H-indene-5-carbonitrile in place of 1-methoxy-5-{[3-(1,3-oxazol-5-yl)-1-{[2-(trimethylsilyl)ethoxy]methyl}-1H-indazol-5-yl]amino}-5,6,7,8-tetrahydronaphthalene-2-carbonitrile, to afford 1-{methyl [3-(1,3-oxazol-5-yl)-1H-indazol-5-yl]amino}-2,3-dihydro-1H-indene-5-carbonitrile (13.5 mg, 92%).

### Step 4: 1-(Methyl(3-(oxazol-5-yl)-1H-indazol-5-yl)amino)-2,3-dihydro-1H-indene-5-carbonitrile, enantiomer 1 and 2

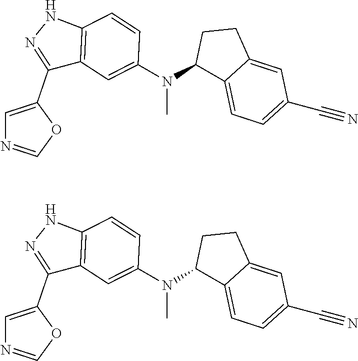 [SMILES: CN(c1ccc2[nH]nc(-c3cnco3)c2c1)[C@H]1CCc2cc(C#N)ccc21 ; CN(c1ccc2[nH]nc(-c3cnco3)c2c1)[C@@H]1CCc2cc(C#N)ccc21]

1-(Methyl(3-(oxazol-5-yl)-1H-indazol-5-yl)amino)-2,3-dihydro-1H-indene-5-carbonitrile was subjected to chiral separation using Method CX to afford 1-(methyl(3-(oxazol-5-yl)-1H-indazol-5-yl)amino)-2,3-dihydro-1H-indene-5-carbonitrile, enantiomer 1 (3.5 mg, 24%) as a white solid. ¹H NMR (400 MHz, MeOD) δ 8.34 (s, 1H), 7.64 (s, 1H), 7.60 (s, 1H), 7.58-7.53 (m, 1H), 7.51 (dd, J=9.2, 0.7 Hz, 1H), 7.42 (dd, J=9.2, 2.3 Hz, 1H), 7.38 (d, J=7.8 Hz, 1H), 7.34 (d, J=2.2 Hz, 1H), 5.65 (t, J=7.9 Hz, 1H), 3.08 (ddd, J=16.6, 9.3, 3.7 Hz, 1H), 3.03-2.91 (m, 1H), 2.71 (s, 3H), 2.49-2.38 (m, 1H), 2.25-2.12 (m, 1H). MS-ESI (m/z) calc'd for C₂₁H₁₈N₅O [M+H]⁺: 356.1. Found 356.1. A later eluting fraction was also isolated to afford 1-(methyl(3-(oxazol-5-yl)-1H-indazol-5-yl)amino)-2,3-dihydro-1H-indene-5-carbonitrile, enantiomer 2 (3.2 mg, 22%) as a white solid. ¹H NMR (400 MHz, MeOD) δ 8.34 (s, 1H), 7.65 (s, 1H), 7.60 (s, 1H), 7.58-7.54 (m, 1H), 7.51 (dd, J=9.2, 0.7 Hz, 1H), 7.43 (dd, J=9.2, 2.3 Hz, 1H), 7.38 (d, J=7.8 Hz, 1H), 7.34 (d, J=2.2 Hz, 1H), 5.66 (t, J=7.9 Hz, 1H), 3.09 (ddd, J=16.6, 9.3, 3.6 Hz, 1H), 3.03-2.90 (m, 1H), 2.72 (s, 3H), 2.51-2.38 (m, 1H), 2.27-2.12 (m, 1H). MS-ESI (m/z) calc'd for C₂₁H₁₈N₅O [M+H]⁺: 356.1. Found 356.1.

### Example 112: 2-Chloro-8-((3-(oxazol-5-yl)-1H-indazol-5-yl)amino)-5,6,7,8-tetrahydroquinoline-3-carbonitrile

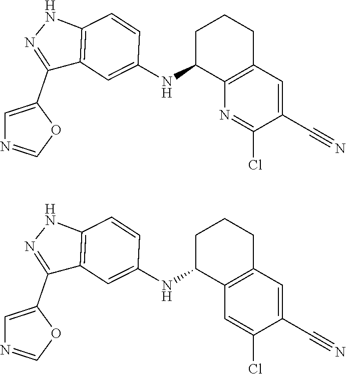 [SMILES: N#Cc1cc2c(nc1Cl)[C@@H](Nc1ccc3[nH]nc(-c4cnco4)c3c1)CCC2 ; N#Cc1cc2c(cc1Cl)[C@H](Nc1ccc3[nH]nc(-c4cnco4)c3c1)CCC2]

### Step 1: 2-Chloro-3-cyano-5,6,7,8-tetrahydroquinoline 1-oxide

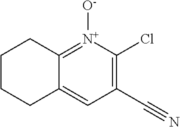 [SMILES: N#Cc1cc2c([n+]([O-])c1Cl)CCCC2]

To a solution of 2-chloro-5,6,7,8-tetrahydroquinoline-3-carbonitrile (2.0 g, 10.38 mmol) in trifluoroacetic acid (50 mL) was added hydrogen peroxide (3.18 mL, 31.15 mmol) and the mixture was stirred at 75° C. for 15 hrs. Water was added and the solution was neutralized by addition of solid K₂CO₃ and extracted with DCM (3×). The combined organic layers were passed through a phase separator and the liquid phase was evaporated to afford the title compound (1.94 g, 90%) as a light yellow solid. ¹H NMR (400 MHz, DMSO-d₆) δ 7.79 (s, 1H) 2.78 (dt, J=16.56, 6.24 Hz, 4H) 1.77-1.86 (m, 2H) 1.63-1.72 (m, 2H). MS-ESI (m/z) calc'd for C₁₀H₁₀ClN[N+][O−] [M+H]⁺: 209.0, Found 209.0.

### Step 2: 2-Chloro-8-hydroxy-5,6,7,8-tetrahydroquinoline-3-carbonitrile

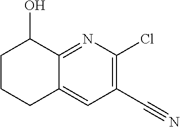 [SMILES: N#Cc1cc2c(nc1Cl)C(O)CCC2]

To a solution of 2-chloro-3-cyano-5,6,7,8-tetrahydroquinoline 1-oxide (1.94 g, 9.3 mmol) in DCM (50 mL) was added trifluoroacetic anhydride (3.88 mL, 27.89 mmol) dropwise and the mixture was stirred at 25° C. for 20 hrs. The solvent was evaporated and the residue was taken up in MeOH. K₂CO₃ was added till basic pH and the suspension was stirred at 25° C. for 1 hr. The solvent was evaporated while maintaining the temperature <40° C. The residue was taken up in water and extracted with DCM (3×). The combined organic layers were passed through a phase separator and evaporated to dryness. The material was purified by chromatography on a 50 g silica gel column, using a 0-10% MeOH/DCM gradient eluent to afford the title compound (0.95 g, 49%) as a white solid. ¹H NMR (400 MHz, DMSO-d₆) δ 8.26 (s, 1H) 5.63 (d, J=5.28 Hz, 1H) 4.56 (q, J=4.84 Hz, 1H) 2.77-2.87 (m, 1H) 2.64-2.75 (m, 1H) 1.80-1.95 (m, 3H) 1.65-1.78 (m, 1H). MS-ESI (m/z) calc'd for C₁₀H₁₀ClN₂O [M+H]⁺: 209.0. Found 209.0.

### Step 3: 2-Chloro-8-oxo-5,6,7,8-tetrahydroquinoline-3-carbonitrile

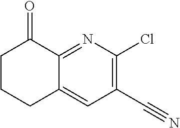 [SMILES: N#Cc1cc2c(nc1Cl)C(=O)CCC2]

A solution of 2-chloro-8-hydroxy-5,6,7,8-tetrahydroquinoline-3-carbonitrile (200.0 mg, 0.960 mmol) in DCM (1.3 mL) was treated with Dess-Martin periodinane (487.9 mg, 1.15 mmol) and stirred at r.t. for 20 hrs. The reaction mixture was diluted with DCM and quenched by addition of 3 mL of saturated aqueous sodium bicarbonate. After stirring at room temperature for 20 minutes, the phases were separated. The aqueous layer was extracted with DCM (2×) and the combined organic phases were washed with water (1×), passed through a phase separator, evaporated and purified by chromatography on a 25 g silica gel column using a 0-80% EtOAc/cyclohexane gradient eluent to afford the title compound (190 mg, 95%) as a beige solid. ¹H NMR (400 MHz, DMSO-d₆) δ 8.65 (s, 1H) 3.02 (t, J=6.05 Hz, 2H) 2.72-2.80 (m, 2H) 2.10 (quin, J=6.38 Hz, 2H). MS-ESI (m/z) calc'd for C₁₀H₈ClN₂O [M+H]⁺: 207.0. Found 207.0.

### Step 4: 2-Chloro-8-((3-iodo-1H-indazol-5-yl)amino)-5,6,7,8-tetrahydroquinoline-3-carbonitrile

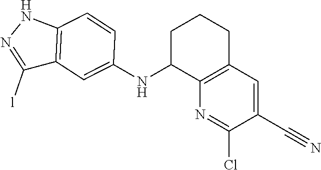

To a stirred solution of 3-iodo-1H-indazol-5-amine (193.62 mg, 0.75 mmol) and 2-chloro-8-oxo-5,6,7,8-tetrahydroquinoline-3-carbonitrile (130.0 mg, 0.62 mmol) in 1,4-dioxane (5 mL) was added 4-methylbenzenesulfonic acid hydrate (11.85 mg, 0.06 mmol) and the mixture was stirred at 100° C. for 4 hrs. The reaction was left to reach r.t. and then heated to 40° C., sodium triacetoxyborohydride (359.25 mg, 1.87 mmol) was added portionwise over 3 hrs and the mixture was left stirring overnight at 40° C. The mixture was cooled to r.t. and sodium borohydride (70.69 mg, 1.87 mmol) was added and the reaction mixture was stirred at r.t. for 48 hrs. The reaction mixture was partitioned between water and EtOAc, the phases were separated, the aqueous layer was extracted with EtOAc (2×) and the combined organic phases washed with water (1×), dried over anhydrous Na₂SO₄ and evaporated to dryness. The material was purified by chromatography on a 20 g silica gel column, using a 0-100% EtOAc/cyclohexane gradient eluent. Selected fractions were collected together and purified again by chromatography on a 25 g NH silica gel column, using a 0-100% EtOAc/cyclohexane gradient eluent to afford the title compound (100 mg, 36%) as a yellow solid. ¹H NMR (400 MHz, DMSO-d₆) δ 13.08 (br. s., 1H) 8.33 (s, 1H) 7.31 (d, J=8.80 Hz, 1H) 6.98 (dd, J=8.91, 1.87 Hz, 1H) 6.52 (d, J=1.54 Hz, 1H) 5.97 (d, J=7.92 Hz, 1H) 4.66 (d, J=7.48 Hz, 1H) 2.75-2.94 (m, 2H) 1.75-1.99 (m, 4H). MS-ESI (m/z) calc'd for C₁₇H₁₄ClIN₅ [M+H]⁺: 450.0. Found 450.0.

### Step 5: 2-Chloro-8-((3-(oxazol-5-yl)-1H-indazol-5-yl)amino)-5,6,7,8-tetrahydroquinoline-3-carbonitrile

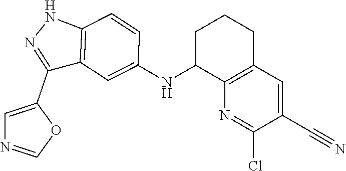 [SMILES: N#Cc1cc2c(nc1Cl)C(Nc1ccc3[nH]nc(-c4cnco4)c3c1)CCC2]

2-Chloro-8-[(3-iodo-1H-indazol-5-yl)amino]-5,6,7,8-tetrahydroquinoline-3-carbonitrile (90.0 mg, 0.20 mmol), oxazole-5-boronic acid pinacol ester (42.93 mg, 0.22 mmol) and KOAc (35.36 mg, 0.36 mmol) were dissolved in 1,4-dioxane (3 mL) and H₂O (1 mL) in a microwave vial. Then the mixture was degassed with N₂ for 5 minutes and 4-di-tert-butylphosphino-N,N-dimethylaniline dichloropalladium (14.17 mg, 0.020 mmol) was added. The vial was sealed and irradiated in a microwave reactor at 100° C. under N₂ for 30 min. The reaction mixture was partitioned between H₂O and EtOAc and the phases were separated. The aqueous layer was extracted with EtOAc (2×) and the combined organic phases were washed with brine (1×), dried over anhydrous Na₂SO₄, and evaporated to dryness. The residue was purified by chromatography on a 10 g silica gel column using a 0-100% EtOAc/cyclohexane gradient eluent to afford the title compound (20 mg, 26%) as a yellow solid.

### Step 6: 2-Chloro-8-((3-(oxazol-5-yl)-1H-indazol-5-yl)amino)-5,6,7,8-tetrahydroquinoline-3-carbonitrile, enantiomer 1 and 2

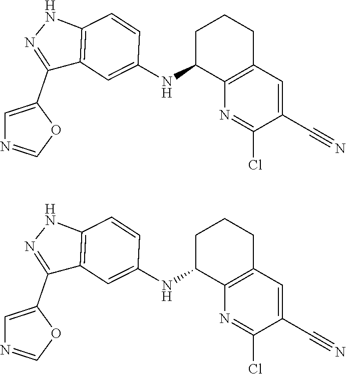 [SMILES: N#Cc1cc2c(nc1Cl)[C@@H](Nc1ccc3[nH]nc(-c4cnco4)c3c1)CCC2 ; N#Cc1cc2c(nc1Cl)[C@H](Nc1ccc3[nH]nc(-c4cnco4)c3c1)CCC2]

2-Chloro-8-((3-(oxazol-5-yl)-1H-indazol-5-yl)amino)-5,6,7,8-tetrahydroquinoline-3-carbonitrile was subjected to chiral preparative HPLC using Method CY. The first eluted enantiomer, 2-chloro-8-((3-(oxazol-5-yl)-1H-indazol-5-yl)amino)-5,6,7,8-tetrahydroquinoline-3-carbonitrile, enantiomer 1 (9.2 mg, 12%) was obtained as a yellow solid. ¹H NMR (400 MHz, DMSO-d₆) δ 13.09 (br. s., 1H) 8.47 (s, 1H) 8.34 (s, 1H) 7.68 (s, 1H) 7.38 (d, J=9.02 Hz, 1H) 7.11 (s, 1H) 6.99 (dd, J=9.02, 1.98 Hz, 1H) 5.99 (d, J=7.70 Hz, 1H) 4.69-4.89 (m, 1H) 2.72-3.00 (m, 2H) 1.73-2.06 (m, 4H). MS-ESI (m/z) calc'd for C₂₀H₁₆ClN₆O [M+H]⁺: 391.1. Found 391.1. A later eluting fraction was also isolated to afford 2-chloro-8-((3-(oxazol-5-yl)-1H-indazol-5-yl)amino)-5,6,7,8-tetrahydroquinoline-3-carbonitrile, enantiomer 2 (9 mg, 12%) as a yellow solid. ¹H NMR (400 MHz, DMSO-d₆) δ 13.10 (br. s., 1H) 8.47 (s, 1H) 8.34 (s, 1H) 7.68 (s, 1H) 7.38 (d, J=9.02 Hz, 1H) 7.11 (d, J=1.32 Hz, 1H) 6.99 (dd, J=9.02, 1.98 Hz, 1H) 5.99 (d, J=7.92 Hz, 1H) 4.74-4.83 (m, 1H) 2.73-2.96 (m, 2H) 1.75-2.05 (m, 4H). MS-ESI (m/z) calc'd for C₂₀H₁₆ClN₆O [M+H]⁺: 391.1. Found 391.1.

### Example 113: 4-Methoxy-1-((3-(oxazol-5-yl)-1H-indazol-5-yl)amino)-2,3-dihydro-1H-indene-5-carbonitrile, enantiomer 1 and 2

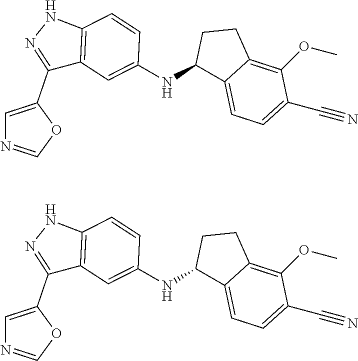 [SMILES: COc1c(C#N)ccc2c1CC[C@@H]2Nc1ccc2[nH]nc(-c3cnco3)c2c1 ; COc1c(C#N)ccc2c1CC[C@H]2Nc1ccc2[nH]nc(-c3cnco3)c2c1]

### Step 1: 4-Methoxy-1-oxo-2,3-dihydro-1H-indene-5-carbonitrile

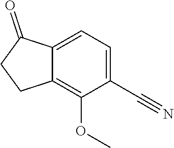

A 0.1 N aqueous solution of potassium hexacyanoferrate (II) (5.07 mL, 0.51 mmol), 5-bromo-4-methoxy-2,3-dihydro-1H-inden-1-one (380.0 mg, 1.58 mmol) and KOAc (309.4 mg, 3.15 mmol) were dissolved in a mixture of 1,4-dioxane (7.62 mL)/H₂O (1.09 mL) in a sealed microwave reaction vial. The mixture was degassed with N₂ for 15 minutes. Then XPhos (75.1 mg, 0.16 mmol) and XPhos-Pd-G3 (133.4 mg, 0.16 mmol) were added and the mixture was left stirring at 110° C. for 2 hrs. The reaction mixture was partitioned between H₂O and EtOAc and the phases were separated. The aqueous layer was extracted with EtOAc (3×) and the combined organic phases were washed with brine (2×), dried over anhydrous Na₂SO₄, filtered, and evaporated to dryness. The material was purified by chromatography on a 25 g silica gel column, using a 0-50% EtOAc/cyclohexane gradient eluent to afford the title compound (65 mg, 22%) as a beige solid. MS-ESI (m/z) calc'd for C₁₁H₁₀NO₂ [M+H]⁺: 187.1. Found 187.9.

### Step 2: 1-((3-Iodo-1H-indazol-5-yl)amino)-4-methoxy-2,3-dihydro-1H-indene-5-carbonitrile

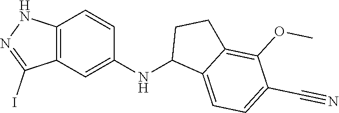 [SMILES: COc1c(C#N)ccc2c1CCC2Nc1ccc2[nH]nc(I)c2c1]

To a stirred solution of 4-methoxy-1-oxo-2,3-dihydro-1H-indene-5-carbonitrile (150.0 mg, 0.61 mmol) and 3-iodo-1H-indazol-5-amine (189.3 mg, 0.73 mmol) in 1,4-dioxane (7.3 mL) was added 4-methylbenzenesulfonic acid hydrate (11.6 mg, 0.06 mmol) and the mixture was stirred at 100° C. for 1.5 hrs. The reaction was cooled to 40° C. and sodium triacetoxyborohydride (120.0 mg, 0.61 mmol) was added portionwise over 1.5 hrs. Then the mixture was left stirring at 40° C. overnight. The reaction mixture was partitioned between H₂O and EtOAc, the phases were separated, the organic layer was washed with saturated aqueous NH₄Cl (1×), dried over anhydrous Na₂SO₄, filtered and evaporated to dryness. The residue was purified by reversed phase chromatography on a 12 g C18 column, using a 2-100% CH₃CN/H₂O with 0.1% formic acid gradient eluent to afford the title compound (43 mg, 16%). ¹H NMR (400 MHz, DMSO-d₆) δ 13.10 (s, 1H), 7.56 (d, J=7.7 Hz, 1H), 7.33 (d, J=8.9 Hz, 1H), 7.11 (d, J=7.8 Hz, 1H), 7.00 (dd, J=9.0, 2.1 Hz, 1H), 6.48 (s, 1H), 6.00 (d, J=8.7 Hz, 1H), 5.08 (q, J=8.0 Hz, 1H), 4.02 (s, 3H), 3.22-3.09 (m, 1H), 3.06-2.95 (m, 1H), 2.61-2.52 (m, 1H), 1.94-1.77 (m, 1H). MS-ESI (m/z) calc'd for C₁₈H₁₆IN₄O [M+H]⁺: 431.0. Found 431.0.

### Step 3: 4-Methoxy-1-((3-(oxazol-5-yl)-1H-indazol-5-yl)amino)-2,3-dihydro-1H-indene-5-carbonitrile

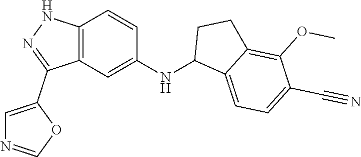 [SMILES: COc1c(C#N)ccc2c1CCC2Nc1ccc2[nH]nc(-c3cnco3)c2c1]

Prepared as described for 2-chloro-8-{[3-(1,3-oxazol-5-yl)-1H-indazol-5-yl]amino}-5,6,7,8-tetrahydroquinoline-3-carbonitrile, using 1-[(3-iodo-1H-indazol-5-yl)amino]-4-methoxy-2,3-dihydro-1H-indene-5-carbonitrile in place of 2-chloro-8-[(3-iodo-1H-indazol-5-yl)amino]-5,6,7,8-tetrahydroquinoline-3-carbonitrile to afford the title compound (12.5 mg, 34%).

### Step 4: 4-Methoxy-1-((3-(oxazol-5-yl)-1H-indazol-5-yl)amino)-2,3-dihydro-1H-indene-5-carbonitrile, enantiomer 1 and 2

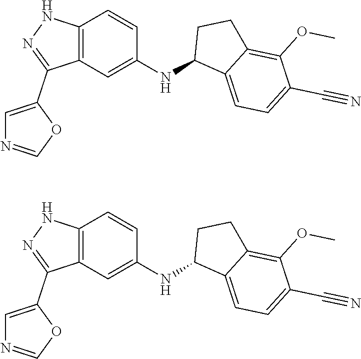 [SMILES: COc1c(C#N)ccc2c1CC[C@@H]2Nc1ccc2[nH]nc(-c3cnco3)c2c1 ; COc1c(C#N)ccc2c1CC[C@H]2Nc1ccc2[nH]nc(-c3cnco3)c2c1]

4-Methoxy-1-((3-(oxazol-5-yl)-1H-indazol-5-yl)amino)-2,3-dihydro-1H-indene-5-carbonitrile was subjected to chiral separation using Method DB to afford 4-methoxy-1-((3-(oxazol-5-yl)-1H-indazol-5-yl)amino)-2,3-dihydro-1H-indene-5-carbonitrile, enantiomer 1 (4.4 mg, 12%) as a white solid. ¹H NMR (400 MHz, DMSO-d₆) δ 13.11 (s, 1H), 8.45 (s, 1H), 7.65 (s, 1H), 7.56 (d, J=7.8 Hz, 1H), 7.39 (d, J=8.9 Hz, 1H), 7.13 (d, J=7.8 Hz, 1H), 7.09 (d, J=2.0 Hz, 1H), 7.00 (dd, J=9.0, 2.1 Hz, 1H), 6.01 (d, J=8.8 Hz, 1H), 5.21 (q, J=8.1 Hz, 1H), 4.02 (s, 3H), 3.16 (ddd, J=15.9, 8.7, 3.2 Hz, 1H), 3.01 (dt, J=16.3, 8.3 Hz, 1H), 2.59 (ddd, J=10.9, 7.7, 3.4 Hz, 1H), 1.87 (dq, J=12.4, 8.6 Hz, 1H). MS-ESI (m/z) calc'd for C₂₁H₁₈N₅O₂ [M+H]⁺: 372.1. Found 372.1. A later eluting fraction was also isolated to afford 4-methoxy-1-((3-(oxazol-5-yl)-1H-indazol-5-yl)amino)-2,3-dihydro-1H-indene-5-carbonitrile, enantiomer 2 (5.1 mg, 14%) as a white solid. ¹H NMR (400 MHz, DMSO-d₆) δ 13.12 (s, 1H), 8.45 (s, 1H), 7.65 (s, 1H), 7.56 (d, J=7.8 Hz, 1H), 7.39 (d, J=9.0 Hz, 1H), 7.13 (d, J=7.8 Hz, 1H), 7.09 (d, J=2.0 Hz, 1H), 7.00 (dd, J=9.0, 2.1 Hz, 1H), 6.01 (d, J=8.9 Hz, 1H), 5.21 (q, J=8.0 Hz, 1H), 4.02 (s, 3H), 3.16 (ddd, J=11.8, 8.8, 4.5 Hz, 1H), 3.01 (dt, J=16.1, 8.0 Hz, 1H), 2.65-2.55 (m, 1H), 1.87 (dq, J=12.4, 8.5 Hz, 1H). MS-ESI (m/z) calc'd for C₂₁H₁₈N₅O₂ [M+H]⁺: 372.1. Found 372.1.

### Example 114: 5-((3-(Isoxazol-4-yl)-1H-indazol-5-yl)amino)-3-methoxy-5,6,7,8-tetrahydronaphthalene-2-carbonitrile, enantiomer 1 and 2

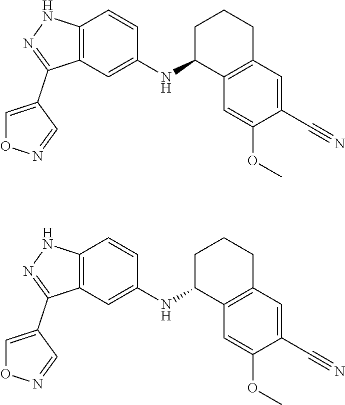 [SMILES: COc1cc2c(cc1C#N)CCC[C@@H]2Nc1ccc2[nH]nc(-c3cnoc3)c2c1 ; COc1cc2c(cc1C#N)CCC[C@H]2Nc1ccc2[nH]nc(-c3cnoc3)c2c1]

### Step 1: 5-((3-Iodo-1H-indazol-5-yl)amino)-3-methoxy-5,6,7,8-tetrahydronaphthalene-2-carbonitrile

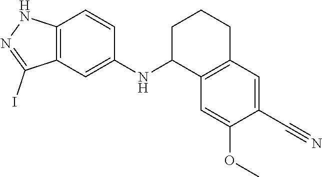 [SMILES: COc1cc2c(cc1C#N)CCCC2Nc1ccc2[nH]nc(I)c2c1]

To a stirred solution of 3-iodo-1H-indazol-5-amine (251.04 mg, 0.97 mmol) and 3-methoxy-5-oxo-7,8-dihydro-6H-naphthalene-2-carbonitrile (130.0 mg, 0.65 mmol) in 1,4-dioxane (5 mL) was added 4-methylbenzenesulfonic acid hydrate (12.29 mg, 0.060 mmol) and the mixture was stirred at 100° C. for 4 hrs. The reaction was cooled to r.t. and then heated to 40° C. Sodium triacetoxyborohydride (372.63 mg, 1.94 mmol) was added portionwise over 3 hrs and the mixture was left stirring for 18 hrs at 40° C. The reaction mixture was then partitioned between H₂O and EtOAc. The phases were separated and the aqueous layer was extracted with EtOAc (2×). The combined organic phases were washed with H₂O (1×), dried over anhydrous Na₂SO₄ and evaporated to dryness. The material was purified by chromatography on a 25 g silica gel column, using a 0-100% EtOAc/cyclohexane gradient eluent. Selected fractions were combined, evaporated to dryness and the residue was purified again by reversed phase chromatography on a 12 g C18 column using a 5-65% CH₃CN/H₂O gradient eluent with 0.1% formic acid to afford the title compound (53 mg, 18%) as a yellow solid. ¹H NMR (400 MHz, DMSO-d₆) δ 13.09 (s, 1H) 7.53 (s, 1H) 7.33 (d, J=9.02 Hz, 1H) 7.19 (s, 1H) 7.03 (dd, J=9.02, 1.98 Hz, 1H) 6.47 (s, 1H) 5.92 (d, J=9.02 Hz, 1H) 4.64-4.74 (m, 1H) 3.79 (s, 3H) 2.62-2.81 (m, 2H) 1.70-1.99 (m, 4H). MS-ESI (m/z) calc'd for C₁₉H₁₈IN₄O [M+H]⁺: 446.0. Found 445.0.

### Step 2: 5-((3-(Isoxazol-4-yl)-1H-indazol-5-yl)amino)-3-methoxy-5,6,7,8-tetrahydronaphthalene-2-carbonitrile, enantiomer 1 and 2

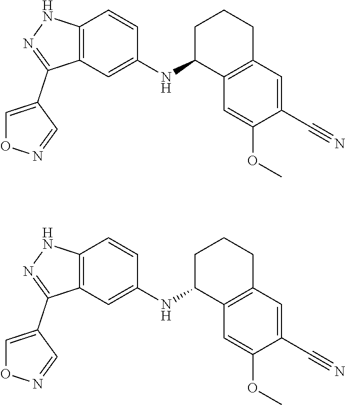 [SMILES: COc1cc2c(cc1C#N)CCC[C@@H]2Nc1ccc2[nH]nc(-c3cnoc3)c2c1 ; COc1cc2c(cc1C#N)CCC[C@H]2Nc1ccc2[nH]nc(-c3cnoc3)c2c1]

Prepared as described for 2-chloro-8-{[3-(1,3-oxazol-5-yl)-1H-indazol-5-yl]amino}-5,6,7,8-tetrahydroquinoline-3-carbonitrile using 5-((3-iodo-1H-indazol-5-yl)amino)-3-methoxy-5,6,7,8-tetrahydronaphthalene-2-carbonitrile in place of 2-chloro-8-[(3-iodo-1H-indazol-5-yl)amino]-5,6,7,8-tetrahydroquinoline-3-carbonitrile. The residue was subjected to chiral separation using Method DC to afford 5-((3-(isoxazol-4-yl)-1H-indazol-5-yl)amino)-3-methoxy-5,6,7,8-tetrahydronaphthalene-2-carbonitrile, enantiomer 1 (10.8 mg, 10%) as a yellow solid. ¹H NMR (400 MHz, DMSO-d₆) δ 13.10 (s, 1H) 8.47 (s, 1H) 7.66 (s, 1H) 7.54 (s, 1H) 7.39 (d, J=8.80 Hz, 1H) 7.21 (s, 1H) 7.08 (s, 1H) 7.03 (dd, J=8.91, 2.09 Hz, 1H) 5.92 (d, J=9.02 Hz, 1H) 4.80 (m, J=4.18 Hz, 1H) 3.78 (s, 3H) 2.65-2.84 (m, 2H) 1.69-2.03 (m, 4H) MS-ESI (m/z) calc'd for C₂₂H₂₀N₅O₂ [M+H]⁺: 386.2. Found 386.2. A later eluting fraction was also isolated to afford 5-((3-(isoxazol-4-yl)-1H-indazol-5-yl)amino)-3-methoxy-5,6,7,8-tetrahydronaphthalene-2-carbonitrile, enantiomer 2 (10.7 mg, 10%) as a yellow solid. ¹H NMR (400 MHz, DMSO-d₆) δ 13.10 (s, 1H) 8.47 (s, 1H) 7.66 (s, 1H) 7.54 (s, 1H) 7.39 (d, J=8.80 Hz, 1H) 7.21 (s, 1H) 7.08 (s, 1H) 7.03 (dd, J=8.91, 2.09 Hz, 1H) 5.92 (d, J=9.24 Hz, 1H) 4.76-4.86 (m, 1H) 3.78 (s, 3H) 2.65-2.84 (m, 2H) 1.71-2.01 (m, 4H). MS-ESI (m/z) calc'd for C₂₂H₂₀N₅O₂ [M+H]⁺: 386.2. Found 386.2.

### Example 115: 8,8-Dimethyl-5-((3-(oxazol-5-yl)-1H-indazol-5-yl)amino)-5,6,7,8-tetrahydronaphthalene-2-carbonitrile, enantiomer 1 and 2

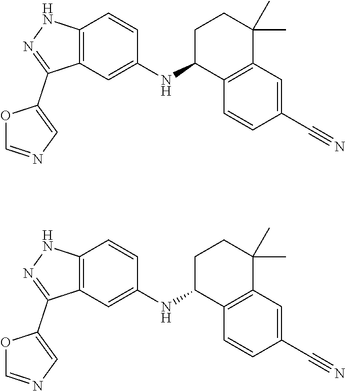 [SMILES: CC1(C)CC[C@H](Nc2ccc3[nH]nc(-c4cnco4)c3c2)c2ccc(C#N)cc21 ; CC1(C)CC[C@@H](Nc2ccc3[nH]nc(-c4cnco4)c3c2)c2ccc(C#N)cc21]

### Step 1: 8,8-Dimethyl-5-oxo-5,6,7,8-tetrahydronaphthalene-2-carbonitrile

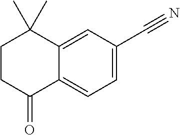

A 0.1 N aqueous solution of potassium hexacyanoferrate (II) (5.14 mL, 0.51 mmol), 6-bromo-4,4-dimethyl-3,4-dihydronaphthalen-1(2H)-one (130.0 mg, 0.51 mmol) and KOAc (50.4 mg, 0.51 mmol) were dissolved in a mixture of 1,4-dioxane (7 mL) and H₂O (1 mL) in a sealed microwave vial. The mixture was degassed with N₂ for 15 minutes. Then XPhos (18.49 mg, 0.04 mmol) and XPhos Pd G3 (32.84 mg, 0.04 mmol) were added and the mixture was stirred at 100° C. for 1 hr. A further amount of 0.08 eq of XPhos and 0.08 eq of XPhos Pd G3 were added and the mixture was stirred at 100° C. for 6 hrs. The reaction mixture was partitioned between water and EtOAc and the phases were separated. The aqueous layer was extracted with EtOAc (2×) and the combined organic phases were washed with H₂O (1×), dried over anhydrous Na₂SO₄, and evaporated to dryness. The residue was purified by chromatography on a 10 g silica gel column, using a 0-15% EtOAc/cyclohexane gradient eluent to afford the title compound (80 mg, 18%) as a light brown solid. ¹H NMR (400 MHz, DMSO-d₆) δ 8.10 (d, J=1.32 Hz, 1H) 7.97 (d, J=8.14 Hz, 1H) 7.79 (dd, J=7.92, 1.54 Hz, 1H) 2.74 (dd, J=7.37, 6.27 Hz, 2H) 1.97-2.02 (m, 2H) 1.38 (s, 6H). MS-ESI (m/z) calc'd for C₁₃H₁₄NO [M+H]⁺: 199.1. Found 200.0.

### Step 2: 5-((3-Iodo-1H-indazol-5-yl)amino)-8,8-dimethyl-5,6,7,8-tetrahydronaphthalene-2-carbonitrile

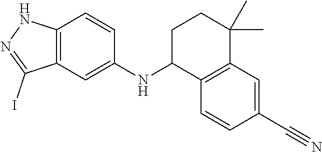 [SMILES: CC1(C)CCC(Nc2ccc3[nH]nc(I)c3c2)c2ccc(C#N)cc21]

To a stirred solution of 3-iodo-1H-indazol-5-amine (156.02 mg, 0.60 mmol) and 8,8-dimethyl-5-oxo-5,6,7,8-tetrahydronaphthalene-2-carbonitrile (80.0 mg, 0.40 mmol) in 1,4-dioxane (3 mL) was added 4-methylbenzenesulfonic acid hydrate (7.64 mg, 0.04 mmol) and the mixture was stirred at 100° C. for 4 hrs. The reaction was warmed to r.t. and then heated to 40° C. Sodium triacetoxyborohydride (231.58 mg, 1.2 mmol) was added portionwise over 2 hrs and the mixture was stirred for 18 hrs at 40° C. The reaction mixture was partitioned between water and EtOAc and the phases were separated. The aqueous layer was extracted with EtOAc (2×) and the combined organic phases were washed with H₂O (1×), dried over anhydrous Na₂SO₄ and evaporated to dryness. The material was purified by chromatography on a 25 g silica gel column using a 0-100% EtOAc/cyclohexane gradient eluent to give material of insufficient purity. The residue was then purified again by reversed phase chromatography on a 12 g C18 column using a 5-70% CH₃CN/H₂O gradient eluent (0.1% formic acid) to afford the title compound (15 mg, 8%) as a yellow solid. ¹H NMR (400 MHz, DMSO-d₆) δ 13.08 (s, 1H) 7.92 (d, J=1.54 Hz, 1H) 7.54-7.59 (m, 1H) 7.49-7.54 (m, 1H) 7.32 (d, J=9.02 Hz, 1H) 7.00 (dd, J=9.02, 1.98 Hz, 1H) 6.42 (s, 1H) 5.98 (d, J=8.80 Hz, 1H) 4.61-4.72 (m, 1H) 1.77-2.04 (m, 3H) 1.65-1.75 (m, 1H) 1.35 (s, 3H) 1.31 (s, 3H) MS-ESI (m/z) calc'd for C₂₀H₂₀IN₄ [M+H]⁺: 443.1. Found 443.1.

### Step 3: 8,8-Dimethyl-5-((3-(oxazol-5-yl)-1H-indazol-5-yl)amino)-5,6,7,8-tetrahydronaphthalene-2-carbonitrile, enantiomer 1 and 2

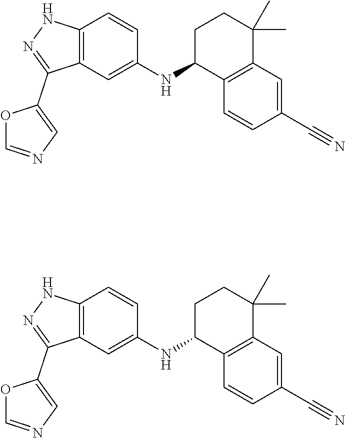 [SMILES: CC1(C)CC[C@H](Nc2ccc3[nH]nc(-c4cnco4)c3c2)c2ccc(C#N)cc21 ; CC1(C)CC[C@@H](Nc2ccc3[nH]nc(-c4cnco4)c3c2)c2ccc(C#N)cc21]

Prepared as described for 2-chloro-8-{[3-(1,3-oxazol-5-yl)-1H-indazol-5-yl]amino}-5,6,7,8-tetrahydroquinoline-3-carbonitrile using 5-((3-Iodo-1H-indazol-5-yl)amino)-8,8-dimethyl-5,6,7,8-tetrahydronaphthalene-2-carbonitrile in place of 2-chloro-8-[(3-iodo-1H-indazol-5-yl)amino]-5,6,7,8-tetrahydroquinoline-3-carbonitrile. The residue was subjected to chiral separation using Method DD to afford 8,8-dimethyl-5-((3-(oxazol-5-yl)-1H-indazol-5-yl)amino)-5,6,7,8-tetrahydronaphthalene-2-carbonitrile, enantiomer 1 (4.6 mg, 35%) as a yellow solid. ¹H NMR (400 MHz, DMSO-d₆) δ 13.10 (br. s., 1H) 8.45 (s, 1H) 7.93 (d, J=1.10 Hz, 1H) 7.65 (s, 1H) 7.52-7.59 (m, 2H) 7.38 (d, J=8.80 Hz, 1H) 6.98-7.06 (m, 2H) 5.98 (d, J=9.02 Hz, 1H) 4.74-4.87 (m, 1H) 1.94-2.06 (m, 1H) 1.78-1.93 (m, 2H) 1.66-1.77 (m, 1H) 1.35 (s, 3H) 1.32 (s, 3H). MS-ESI (m/z) calc'd for C₂₃H₂₂N₅O [M+H]⁺: 384.2. Found 384.2. A later eluting fraction was also isolated to afford 8,8-dimethyl-5-((3-(oxazol-5-yl)-1H-indazol-5-yl)amino)-5,6,7,8-tetrahydronaphthalene-2-carbonitrile, enantiomer 2 (3.7 mg, 28%) as a yellow solid. ¹H NMR (400 MHz, DMSO-d₆) δ 13.09 (s, 1H) 8.45 (s, 1H) 7.93 (d, J=0.88 Hz, 1H) 7.65 (s, 1H) 7.52-7.59 (m, 2H) 7.38 (d, J=8.80 Hz, 1H) 6.98-7.07 (m, 2H) 5.98 (d, J=9.02 Hz, 1H) 4.74-4.87 (m, 1H) 1.95-2.05 (m, 1H) 1.78-1.94 (m, 2H) 1.64-1.77 (m, 1H) 1.35 (s, 3H) 1.32 (s, 3H). MS-ESI (m/z) calc'd for C₂₃H₂₂N₅O [M+H]⁺: 384.2. Found 384.2.

### Example 116: 5-((3-(2-Ethylpyridin-4-yl)-1H-indazol-5-yl)amino)-5,6,7,8-tetrahydronaphthalene-2-carbonitrile, enantiomer 1 and 2

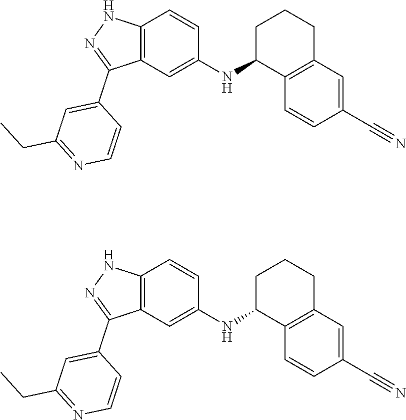 [SMILES: CCc1cc(-c2n[nH]c3ccc(N[C@H]4CCCc5cc(C#N)ccc54)cc23)ccn1 ; CCc1cc(-c2n[nH]c3ccc(N[C@@H]4CCCc5cc(C#N)ccc54)cc23)ccn1]

### Step 1: 5-((3-(2-Ethylpyridin-4-yl)-1H-indazol-5-yl)amino)-5,6,7,8-tetrahydronaphthalene-2-carbonitrile

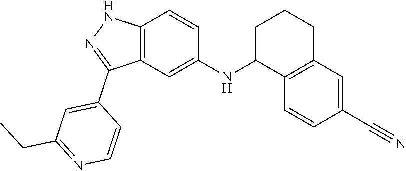 [SMILES: CCc1cc(-c2n[nH]c3ccc(NC4CCCc5cc(C#N)ccc54)cc23)ccn1]

Prepared as described for 2-chloro-8-{[3-(1,3-oxazol-5-yl)-1H-indazol-5-yl]amino}-5,6,7,8-tetrahydroquinoline-3-carbonitrile using 5-((3-iodo-1H-indazol-5-yl)amino)-5,6,7,8-tetrahydronaphthalene-2-carbonitrile in place of 2-chloro-8-[(3-iodo-1H-indazol-5-yl)amino]-5,6,7,8-tetrahydroquinoline-3-carbonitrile, and 2-ethyl-4-(4,4,5,5-tetramethyl-1,3,2-dioxaborolan-2-yl)pyridine in place of 5-(4,4,5,5-tetramethyl-1,3,2-dioxaborolan-2-yl)-1,3-oxazole to afford the title compound (65 mg, 91%).

### Step 2: 5-((3-(2-Ethylpyridin-4-yl)-1H-indazol-5-yl)amino)-5,6,7,8-tetrahydronaphthalene-2-carbonitrile, enantiomer 1 and 2

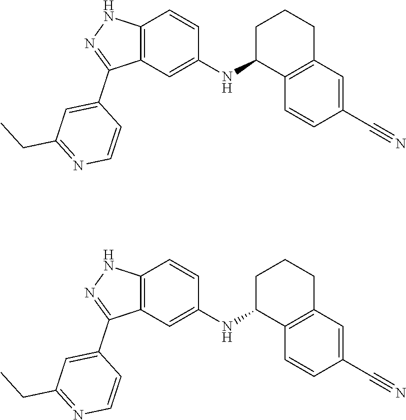 [SMILES: CCc1cc(-c2n[nH]c3ccc(N[C@H]4CCCc5cc(C#N)ccc54)cc23)ccn1 ; CCc1cc(-c2n[nH]c3ccc(N[C@@H]4CCCc5cc(C#N)ccc54)cc23)ccn1]

5-((3-(2-Ethylpyridin-4-yl)-1H-indazol-5-yl)amino)-5,6,7,8-tetrahydronaphthalene-2-carbonitrile was subjected to chiral separation using Method DE to afford 5-((3-(2-ethylpyridin-4-yl)-1H-indazol-5-yl)amino)-5,6,7,8-tetrahydronaphthalene-2-carbonitrile, enantiomer 1 (15.0 mg, 21%) as a yellow solid. ¹H NMR (400 MHz, DMSO-d₆) δ 13.16 (s, 1H) 8.48-8.56 (m, 1H) 7.63-7.72 (m, 3H) 7.52-7.62 (m, 2H) 7.41 (d, J=8.80 Hz, 1H) 7.14 (s, 1H) 7.03 (dd, J=9.02, 1.98 Hz, 1H) 5.98 (d, J=9.02 Hz, 1H) 4.81 (m, J=6.82 Hz, 1H) 2.75-2.91 (m, 4H) 1.76-2.05 (m, 4H) 1.27 (t, J=7.59 Hz, 3H). MS-ESI (m/z) calc'd for C₂₅H₂₄N₅ [M+H]⁺: 394.2. Found 394.2. A later eluting fraction was also isolated to afford 5-((3-(2-ethylpyridin-4-yl)-1H-indazol-5-yl)amino)-5,6,7,8-tetrahydronaphthalene-2-carbonitrile, enantiomer 2 (14.9 mg, 21%) as a yellow solid. ¹H NMR (400 MHz, DMSO-d₆) δ 13.16 (s, 1H) 8.48-8.56 (m, 1H) 7.64-7.72 (m, 3H) 7.53-7.62 (m, 2H) 7.41 (d, J=8.80 Hz, 1H) 7.14 (s, 1H) 7.03 (dd, J=9.02, 1.98 Hz, 1H) 5.99 (d, J=9.02 Hz, 1H) 4.76-4.88 (m, 1H) 2.75-2.91 (m, 4H) 1.78-2.04 (m, 4H) 1.27 (t, J=7.59 Hz, 3H) MS-ESI (m/z) calc'd for C₂₅H₂₄N₅ [M+H]⁺: 394.2. Found 394.2.

### Example 117: 5-((3-(2-Methoxypyridin-4-yl)-1H-indazol-5-yl)amino)-5,6,7,8-tetrahydronaphthalene-2-carbonitrile, enantiomer 1 and 2

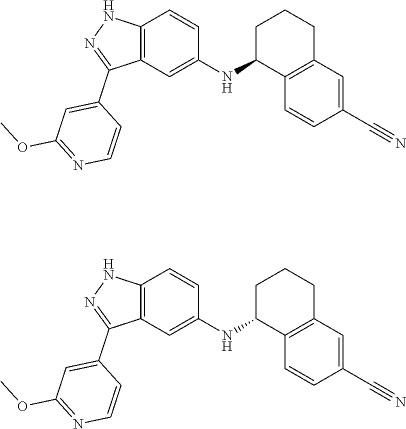 [SMILES: COc1cc(-c2n[nH]c3ccc(N[C@H]4CCCc5cc(C#N)ccc54)cc23)ccn1 ; COc1cc(-c2n[nH]c3ccc(N[C@@H]4CCCc5cc(C#N)ccc54)cc23)ccn1]

Prepared as described for 2-chloro-8-{[3-(1,3-oxazol-5-yl)-1H-indazol-5-yl]amino}-5,6,7,8-tetrahydroquinoline-3-carbonitrile using 5-[(3-iodo-1H-indazol-5-yl)amino]-5,6,7,8-tetrahydronaphthalene-2-carbonitrile in place of 2-chloro-8-[(3-iodo-1H-indazol-5-yl)amino]-5,6,7,8-tetrahydroquinoline-3-carbonitrile, and 2-methoxy-4-(4,4,5,5-tetramethyl-1,3,2-dioxaborolan-2-yl)pyridine in place of 5-(4,4,5,5-tetramethyl-1,3,2-dioxaborolan-2-yl)-1,3-oxazole. The residue (50 mg, 70%) was subjected to chiral separation using Method DF to afford 5-[[3-(2-methoxypyridin-4-yl)-1H-indazol-5-yl]amino]-5,6,7,8-tetrahydronaphthalene-2-carbonitrile, enantiomer 1 (20.0 mg, 28%) as a yellow solid. ¹H NMR (400 MHz, DMSO-d₆) δ 13.18 (br. s., 1H) 8.22 (d, J=5.94 Hz, 1H) 7.65 (s, 1H) 7.49-7.61 (m, 3H) 7.41 (d, J=8.80 Hz, 1H) 7.21 (d, J=0.66 Hz, 1H) 7.13 (s, 1H) 7.02 (dd, J=8.91, 1.87 Hz, 1H) 5.97 (d, J=9.02 Hz, 1H) 4.76-4.88 (m, 1H) 3.90 (s, 3H) 2.75-2.95 (m, 2H) 1.75-2.04 (m, 4H) MS-ESI (m/z) calc'd for C₂₄H₂₂N₅O [M+H]⁺: 396.2. Found 396.2. A later eluting fraction was also isolated to afford 5-[[3-(2-methoxypyridin-4-yl)-1H-indazol-5-yl]amino]-5,6,7,8-tetrahydronaphthalene-2-carbonitrile, enantiomer 2 (20.0 mg, 28%) as a yellow solid. ¹H NMR (400 MHz, DMSO-d₆) δ 13.18 (br. s., 1H) 8.18-8.25 (m, 1H) 7.65 (s, 1H) 7.49-7.61 (m, 3H) 7.41 (d, J=8.80 Hz, 1H) 7.21 (d, J=0.66 Hz, 1H) 7.13 (s, 1H) 7.02 (dd, J=9.02, 1.98 Hz, 1H) 5.97 (d, J=9.02 Hz, 1H) 4.82 (m, J=5.06 Hz, 1H) 3.90 (s, 3H) 2.75-2.90 (m, 2H) 1.75-2.03 (m, 4H). MS-ESI (m/z) calc'd for C₂₄H₂₂N₅O [M+H]⁺: 396.2. Found 396.2.

### Example 118: 1-((3-(Isoxazol-4-yl)-1H-indazol-5-yl)amino)-4-methyl-2,3-dihydro-1H-indene-5-carbonitrile, enantiomer 1 and 2

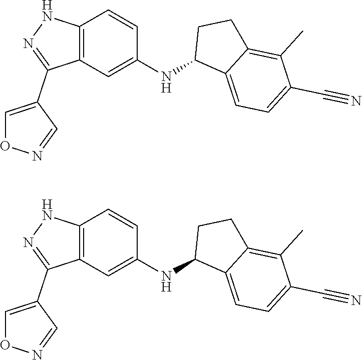 [SMILES: Cc1c(C#N)ccc2c1CC[C@H]2Nc1ccc2[nH]nc(-c3cnoc3)c2c1 ; Cc1c(C#N)ccc2c1CC[C@@H]2Nc1ccc2[nH]nc(-c3cnoc3)c2c1]

Prepared as described for 2-chloro-8-{[3-(1,3-oxazol-5-yl)-1H-indazol-5-yl]amino}-5,6,7,8-tetrahydroquinoline-3-carbonitrile, using 1-((3-iodo-1H-indazol-5-yl)amino)-4-methyl-2,3-dihydro-1H-indene-5-carbonitrile in place of 2-chloro-8-[(3-iodo-1H-indazol-5-yl)amino]-5,6,7,8-tetrahydroquinoline-3-carbonitrile and (1,2-oxazol-4-yl)boronic acid in place of oxazole-5-boronic acid pinacol ester to afford the title compound (40 mg, 52%) as a racemic mixture. The residue was purified by chiral separation using Method DG to afford 1-((3-(isoxazol-4-yl)-1H-indazol-5-yl)amino)-4-methyl-2,3-dihydro-1H-indene-5-carbonitrile, enantiomer 1 (11.5 mg, 15%) as a white solid. ¹H NMR (400 MHz, DMSO-d₆) δ 12.90 (s, 1H), 9.60 (s, 1H), 9.11 (s, 1H), 7.58 (d, J=7.8 Hz, 1H), 7.35 (d, J=8.9 Hz, 1H), 7.29 (d, J=7.8 Hz, 1H), 7.10 (d, J=2.0 Hz, 1H), 6.97 (dd, J=9.0, 2.0 Hz, 1H), 5.87 (d, J=9.1 Hz, 1H), 5.33 (q, J=7.9 Hz, 1H), 3.08-2.94 (m, 1H), 2.90-2.76 (m, 1H), 2.72-2.60 (m, 1H), 2.44 (s, 3H), 1.91-1.76 (m, 1H). MS-ESI (m/z) calc'd for C₂₁H₁₈N₅O [M+H]⁺: 356.1. Found 356.2. A later eluting fraction was also isolated to afford 1-((3-(isoxazol-4-yl)-1H-indazol-5-yl)amino)-4-methyl-2,3-dihydro-1H-indene-5-carbonitrile, enantiomer 2 (11 mg, 14%) as a beige solid. ¹H NMR (400 MHz, DMSO-d₆) δ 12.90 (s, 1H), 9.60 (s, 1H), 9.11 (s, 1H), 7.58 (d, J=7.8 Hz, 1H), 7.35 (d, J=8.9 Hz, 1H), 7.29 (d, J=7.8 Hz, 1H), 7.10 (d, J=2.0 Hz, 1H), 6.97 (dd, J=8.9, 2.1 Hz, 1H), 5.87 (d, J=9.1 Hz, 1H), 5.33 (q, J=8.0 Hz, 1H), 3.07-2.94 (m, 1H), 2.90-2.76 (m, 1H), 2.73-2.59 (m, 1H), 2.44 (s, 3H), 1.91-1.75 (m, 1H). MS-ESI (m/z) calc'd for C₂₁H₁₈N₅O [M+H]⁺: 356.1. Found 356.2.

### Example 119: 5-((3-(Oxazol-5-yl)-1H-indazol-5-yl)amino)-5,6,7,8-tetrahydronaphthalene-2-carbonitrile, enantiomer 1 and 2

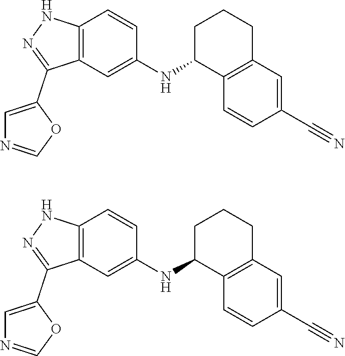 [SMILES: N#Cc1ccc2c(c1)CCC[C@H]2Nc1ccc2[nH]nc(-c3cnco3)c2c1 ; N#Cc1ccc2c(c1)CCC[C@@H]2Nc1ccc2[nH]nc(-c3cnco3)c2c1]

Prepared as described for 2-chloro-8-{[3-(1,3-oxazol-5-yl)-1H-indazol-5-yl]amino}-5,6,7,8-tetrahydroquinoline-3-carbonitrile using 5-((3-iodo-1H-indazol-5-yl)amino)-5,6,7,8-tetrahydronaphthalene-2-carbonitrile in place of 2-chloro-8-[(3-iodo-1H-indazol-5-yl)amino]-5,6,7,8-tetrahydroquinoline-3-carbonitrile to afford 5-{[3-(1,3-oxazol-5-yl)-1H-indazol-5-yl]amino}-5,6,7,8-tetrahydronaphthalene-2-carbonitrile (80 mg) which was subjected to chiral separation using Method DH to afford 5-((3-(oxazol-5-yl)-1H-indazol-5-yl)amino)-5,6,7,8-tetrahydronaphthalene-2-carbonitrile, enantiomer 1 (19.9 mg, 30%) as an off-white solid. ¹H NMR (400 MHz, DMSO-d₆) δ 13.09 (br. s., 1H) 8.46 (s, 1H) 7.63-7.68 (m, 2H) 7.52-7.60 (m, 2H) 7.38 (d, J=9.02 Hz, 1H) 6.99-7.08 (m, 2H) 5.96 (d, J=9.02 Hz, 1H) 4.83 (m, J=8.14 Hz, 1H) 2.75-2.91 (m, 2H) 1.78-2.04 (m, 4H). MS-ESI (m/z) calc'd for C₂₁H₁₈N₅O [M+H]⁺: 356.1. Found 356.1. A later eluting fraction was also isolated to afford 5-((3-(oxazol-5-yl)-1H-indazol-5-yl)amino)-5,6,7,8-tetrahydronaphthalene-2-carbonitrile, enantiomer 2 (23.1 mg, 34%) as an off-white solid. ¹H NMR (400 MHz, DMSO-d₆) δ 13.09 (br. s., 1H) 8.45 (s, 1H) 7.62-7.68 (m, 2H) 7.48-7.61 (m, 2H) 7.38 (d, J=9.02 Hz, 1H) 6.95-7.09 (m, 2H) 5.96 (d, J=9.24 Hz, 1H) 4.82 (m, J=9.02 Hz, 1H) 2.73-2.96 (m, 2H) 1.78-2.04 (m, 4H). MS-ESI (m/z) calc'd for C₂₁H₁₈N₅O [M+H]⁺: 356.1. Found 356.1.

### Example 120: 8-((3-(Oxazol-5-yl)-1H-indazol-5-yl)amino)-5,6,7,8-tetrahydroquinoline-3-carbonitrile, enantiomer 1 and 2

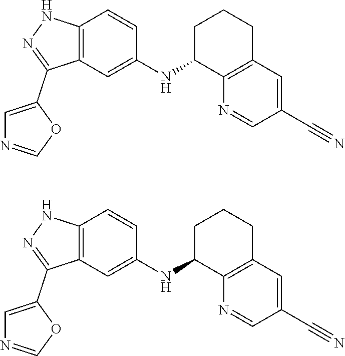 [SMILES: N#Cc1cnc2c(c1)CCC[C@H]2Nc1ccc2[nH]nc(-c3cnco3)c2c1 ; N#Cc1cnc2c(c1)CCC[C@@H]2Nc1ccc2[nH]nc(-c3cnco3)c2c1]

### Step 1: 8-((3-Iodo-1H-indazol-5-yl)amino)-5,6,7,8-tetrahydroquinoline-3-carbonitrile

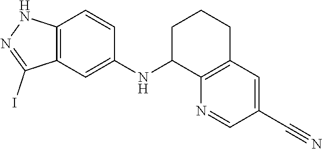 [SMILES: N#Cc1cnc2c(c1)CCCC2Nc1ccc2[nH]nc(I)c2c1]

To a solution of 3-iodo-1H-indazol-5-amine (282.1 mg, 1.09 mmol) and 8-oxo-5,6,7,8-tetrahydroquinoline-3-carbonitrile (125.0 mg, 0.73 mmol) in 1,4-dioxane (5 mL) was added p-toluensulfonic acid monohydrate (13.8 mg, 0.07 mmol) and the mixture was stirred at 100° C. for 3 hrs. The reaction was cooled to 40° C. and sodium triacetoxyborohydride (558.3 mg, 2.90 mmol) was added portionwise over 3 hrs. The mixture was then stirred at 40° C. overnight. The reaction mixture was partitioned between H₂O and EtOAc and the phases were separated. The aqueous layer was extracted with EtOAc (2×) and the combined organic phases were washed with H₂O (1×), dried over anhydrous Na₂SO₄, and evaporated to dryness. The residue was purified by chromatography on a 28 g NH silica gel column, using a 0-100% EtOAc/cyclohexane gradient eluent to give material of insufficient purity. The residue was then purified again by reversed phase chromatography on a 30 g C18 column, using a 5-50% CH₃CN/H₂O gradient eluent (0.1% formic acid) to afford the title compound (75 mg, 25%) as a yellow solid. ¹H NMR (400 MHz, DMSO-d₆) δ 13.07 (s, 1H) 8.81 (d, J=1.98 Hz, 1H) 8.13 (d, J=1.98 Hz, 1H) 7.30 (d, J=9.02 Hz, 1H) 7.00 (dd, J=8.91, 2.09 Hz, 1H) 6.49 (s, 1H) 5.93 (d, J=7.48 Hz, 1H) 4.60-4.74 (m, 1H) 2.75-3.00 (m, 2H) 1.76-2.13 (m, 4H). MS-ESI (m/z) calc'd for C₁₇H₁₅IN₅ [M+H]⁺: 416.0. Found 416.0.

### Step 2: 8-((3-(Oxazol-5-yl)-1H-indazol-5-yl)amino)-5,6,7,8-tetrahydroquinoline-3-carbonitrile, enantiomer 1 and 2

Prepared as described for 2-chloro-8-([3-(1,3-oxazol-5-yl)-1H-indazol-5-yl]amino)-5,6,7,8-tetrahydroquinoline-3-carbonitrile using 8-((3-iodo-1H-indazol-5-yl)amino)-5,6,7,8-tetrahydroquinoline-3-carbonitrile in place of 2-chloro-8-[(3-iodo-1H-indazol-5-yl)amino]-5,6,7,8-tetrahydroquinoline-3-carbonitrile to afford a racemic mixture of the title compound (48 mg, 75%). The mixture was purified by chiral separation using Method DI afford 8-((3-(oxazol-5-yl)-1H-indazol-5-yl)amino)-5,6,7,8-tetrahydroquinoline-3-carbonitrile, enantiomer 1 (12.5 mg, 19%) as a yellow solid. ¹H NMR (400 MHz, DMSO-d₆) δ 13.07 (br. s., 1H) 8.81 (d, J=2.20 Hz, 1H) 8.47 (s, 1H) 8.14 (d, J=1.98 Hz, 1H) 7.65 (s, 1H) 7.36 (d, J=9.02 Hz, 1H) 7.09 (s, 1H) 7.00 (dd, J=8.91, 2.09 Hz, 1H) 5.94 (d, J=7.26 Hz, 1H) 4.73-4.85 (m, 1H) 2.75-2.99 (m, 2H) 1.79-2.16 (m, 4H). MS-ESI (m/z) calc'd for C₂₀H₁₇N₆₀ [M+H]⁺: 357.1. Found 357.1. A later eluting fraction was also isolated to afford 8-((3-(oxazol-5-yl)-1H-indazol-5-yl)amino)-5,6,7,8-tetrahydroquinoline-3-carbonitrile, enantiomer 2 (14.8 mg, 23%) as a yellow solid. ¹H NMR (400 MHz, DMSO-d₆) δ 13.08 (br. s., 1H) 8.81 (d, J=1.98 Hz, 1H) 8.47 (s, 1H) 8.14 (d, J=1.98 Hz, 1H) 7.65 (s, 1H) 7.36 (d, J=8.80 Hz, 1H) 7.09 (d, J=1.54 Hz, 1H) 7.00 (dd, J=8.91, 2.09 Hz, 1H) 5.93 (d, J=7.48 Hz, 1H) 4.78 (q, J=5.80 Hz, 1H) 2.76-2.99 (m, 2H) 1.76-2.15 (m, 4H). MS-ESI (m/z) calc'd for C₂₀H₁₇N₆O [M+H]⁺: 357.1. Found 357.1.

### Example 121: 8-((3-(2-Ethylpyridin-4-yl)-1H-indazol-5-yl)oxy)-5,6,7,8-tetrahydroquinoline-3-carbonitrile, enantiomer 1 and 2

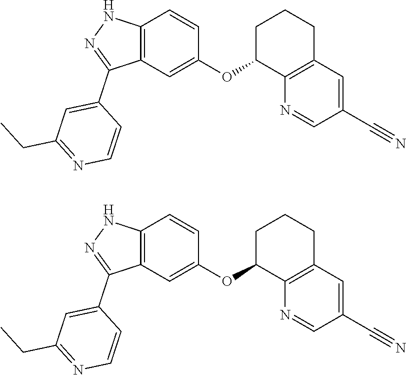 [SMILES: CCc1cc(-c2n[nH]c3ccc(O[C@@H]4CCCc5cc(C#N)cnc54)cc23)ccn1 ; CCc1cc(-c2n[nH]c3ccc(O[C@H]4CCCc5cc(C#N)cnc54)cc23)ccn1]

### Step 1: 8-Hydroxy-5,6,7,8-tetrahydroquinoline-3-carbonitrile

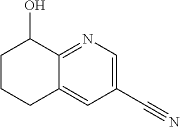 [SMILES: N#Cc1cnc2c(c1)CCCC2O]

8-Oxo-5,6,7,8-tetrahydroquinoline-3-carbonitrile (250.0 mg, 1.45 mmol) was dissolved in MeOH (25 mL), then sodium borohydride (109.86 mg, 2.9 mmol) was added and the mixture was allowed to stir at r.t. for 30 min. The reaction mixture was partitioned between H₂O and EtOAc and the phases were separated. The aqueous layer was extracted with EtOAc (2×) and the combined organic phases were washed with H₂O, dried over anhydrous Na₂SO₄, and evaporated to dryness to afford the title compound (205 mg, 81%) as a yellow oil. ¹H NMR (400 MHz, DMSO-d₆) δ 8.73-8.91 (m, 1H) 7.99-8.13 (m, 1H) 5.40 (d, J=4.40 Hz, 1H) 4.60 (q, J=4.25 Hz, 1H) 2.66-2.91 (m, 2H) 1.62-1.97 (m, 4H). MS-ESI (m/z) calc'd for C₁₀H₁₁N₂O [M+H]⁺: 175.1. Found 175.0.

### Step 2: tert-Butyl 5-((3-cyano-5,6,7,8-tetrahydroquinolin-8-yl)oxy)-3-iodo-1H-indazole-1-carboxylate

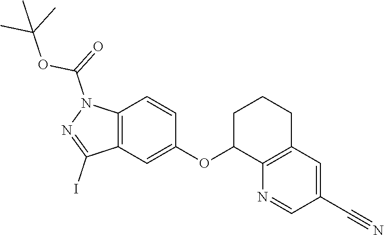 [SMILES: CC(C)(C)OC(=O)n1nc(I)c2cc(OC3CCCc4cc(C#N)cnc43)ccc21]

Diethyl azodicarboxylate (0.18 mL, 1.15 mmol) was added to a solution of rac-8-hydroxy-5,6,7,8-tetrahydroquinoline-3-carbonitrile (200.0 mg, 1.15 mmol), tert-butyl 5-hydroxy-3-iodo-1H-indazole-1-carboxylate (413.49 mg, 1.15 mmol) and triphenylphosphine (331.25 mg, 1.26 mmol) in THF (5 mL) at r.t. The reaction mixture was allowed to stir for 15 minutes at 0° C., and then warmed to r.t. and stirred for 2 hrs. The reaction mixture was partitioned between H₂O and EtOAc and the phases were separated. The aqueous layer was extracted with EtOAc (2×) and the combined organic phases were washed with H₂O (1×), dried over anhydrous Na₂SO₄, and evaporated to dryness. The residue was purified by chromatography on a 25 g silica gel column, using a 0-30% EtOAc/cyclohexane gradient eluent to give material of insufficient purity. The residue was purified again by chromatography on a 28 g NH silica gel column, a 0-20% EtOAc/cyclohexane gradient eluent to afford the title compound (330 mg, 56%), as a white solid. ¹H NMR (400 MHz, DMSO-d₆) δ 8.85 (d, J=1.98 Hz, 1H) 8.21 (d, J=1.98 Hz, 1H) 7.98 (d, J=9.24 Hz, 1H) 7.41 (dd, J=9.24, 2.42 Hz, 1H) 7.22 (d, J=2.20 Hz, 1H) 5.67 (t, J=4.07 Hz, 1H) 2.91-3.07 (m, 1H) 2.77-2.90 (m, 1H) 2.23 (m, J=6.60 Hz, 1H) 2.02-2.15 (m, 1H) 1.78-2.01 (m, 2H) 1.65 (s, 9H). MS-ESI (m/z) calc'd for C₂₂H₂₂IN₄O₃ [M+H]⁺: 517.0. Found 394.1, 517.0.

### Step 3: 8-((3-(2-Ethylpyridin-4-yl)-1H-indazol-5-yl)oxy)-5,6,7,8-tetrahydroquinoline-3-carbonitrile

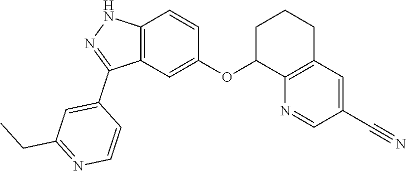 [SMILES: CCc1cc(-c2n[nH]c3ccc(OC4CCCc5cc(C#N)cnc54)cc23)ccn1]

tert-Butyl-5-((3-cyano-5,6,7,8-tetrahydroquinolin-8-yl)oxy)-3-iodo-1H-indazole-1-carboxylate (75.0 mg, 0.15 mmol), 2-ethyl-4-(4,4,5,5-tetramethyl-1,3,2-dioxaborolan-2-yl)pyridine (37.2 mg, 0.16 mmol) and KOAc (25.7 mg, 0.26 mmol) were dissolved in 1,4-dioxane (2 mL) and water (0.5 mL) in a microwave vial. The mixture was degassed with N₂ for 5 minutes and Pd(amphos)Cl₂ (10.3 mg, 0.01 mmol) was added. The vial was sealed and the mixture irradiated in a microwave reactor at 100° C. under N₂ for 30 min. The reaction mixture was partitioned between H₂O and EtOAc and the phases were separated. The aqueous layer was extracted with EtOAc (2×) and the combined organic phases were washed with H₂O (1×), dried over Na₂SO₄, and evaporated to dryness. The residue was purified by chromatography on a 5 g silica gel column, using a 0-100% EtOAc/cyclohexane gradient eluent to afford the title compound (30 mg, 52%) as a yellow solid.

### Step 4: 8-((3-(2-Ethylpyridin-4-yl)-1H-indazol-5-yl)oxy)-5,6,7,8-tetrahydroquinoline-3-carbonitrile, enantiomer 1 and 2

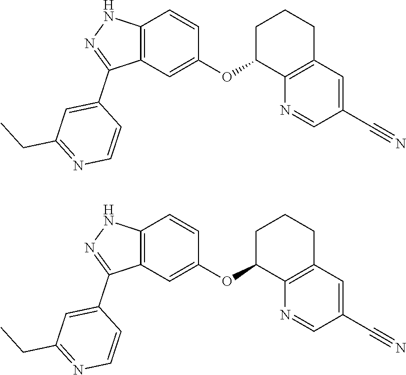 [SMILES: CCc1cc(-c2n[nH]c3ccc(O[C@@H]4CCCc5cc(C#N)cnc54)cc23)ccn1 ; CCc1cc(-c2n[nH]c3ccc(O[C@H]4CCCc5cc(C#N)cnc54)cc23)ccn1]

The residue was purified by chiral separation using Method DJ to afford 8-((3-(2-ethylpyridin-4-yl)-1H-indazol-5-yl)oxy)-5,6,7,8-tetrahydroquinoline-3-carbonitrile, enantiomer 1 (13.4 mg, 23%) as a light yellow solid. ¹H NMR (400 MHz, DMSO-d₆) δ 13.43 (s, 1H) 8.87 (d, J=1.98 Hz, 1H) 8.58 (d, J=5.06 Hz, 1H) 8.22 (d, J=1.98 Hz, 1H) 7.76-7.85 (m, 3H) 7.57 (d, J=9.02 Hz, 1H), 7.22 (dd, J=9.02, 1.98 Hz, 1H), 5.61-5.71 (m, 1H) 2.93-3.04 (m, 1H) 2.77-2.90 (m, 3H), 2.24-2.32 (m, 1H), 1.91-2.08 (m, 2H), 1.77-1.90 (m, 1H) 1.30 (t, J=7.59 Hz, 3H). MS-ESI (m/z) calc'd for C₂₄H₂₂N₅O [M+H]⁺: 396.2. Found 396.2. A later eluting fraction was also isolated to afford 8-((3-(2-ethylpyridin-4-yl)-1H-indazol-5-yl)oxy)-5,6,7,8-tetrahydroquinoline-3-carbonitrile, enantiomer 2 (13.4 mg, 23%) as a light yellow solid. ¹H NMR (400 MHz, DMSO-d₆) δ 13.43 (s, 1H) 8.87 (d, J=1.98 Hz, 1H) 8.58 (d, J=5.06 Hz, 1H) 8.22 (d, J=1.98 Hz, 1H) 7.75-7.86 (m, 3H) 7.57 (d, J=9.02 Hz, 1H) 7.22 (dd, J=9.02, 2.20 Hz, 1H) 5.61-5.72 (m, 1H) 2.93-3.05 (m, 1H) 2.77-2.92 (m, 3H) 2.24-2.33 (m, 1H) 1.91-2.07 (m, 2H) 1.77-1.90 (m, 1H) 1.30 (t, J=7.59 Hz, 3H). MS-ESI (m/z) calc'd for C₂₄H₂₂N₅O [M+H]⁺: 396.2. Found 396.2.

### Example 122: 8-((3-(Oxazol-5-yl)-1H-indazol-5-yl)oxy)-5,6,7,8-tetrahydroisoquinoline-3-carbonitrile, enantiomer 1 and 2

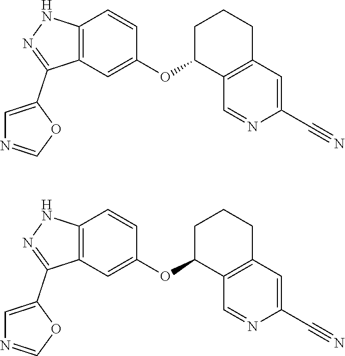 [SMILES: N#Cc1cc2c(cn1)[C@H](Oc1ccc3[nH]nc(-c4cnco4)c3c1)CCC2 ; N#Cc1cc2c(cn1)[C@@H](Oc1ccc3[nH]nc(-c4cnco4)c3c1)CCC2]

### Step 1: tert-Butyl 5-((3-cyano-5,6,7,8-tetrahydroisoquinolin-8-yl)oxy)-3-iodo-1H-indazole-1-carboxylate

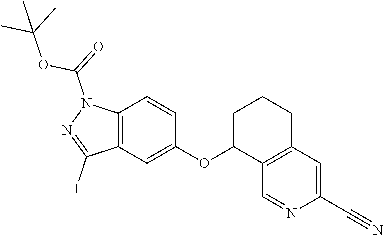 [SMILES: CC(C)(C)OC(=O)n1nc(I)c2cc(OC3CCCc4cc(C#N)ncc43)ccc21]

Diethyl azodicarboxylate (219.9 mg, 1.26 mmol) was added dropwise to a stirred solution of 5-hydroxy-5,6,7,8-tetrahydroquinoline-2-carbonitrile (220.0 mg, 1.26 mmol), tert-butyl 5-hydroxy-3-iodo-1H-indazole-1-carboxylate (454.8 mg, 1.26 mmol) and triphenylphosphine (364.4 mg, 1.39 mmol) in THF (5.5 mL) at 0° C. The reaction mixture was stirred for 15 minutes at 0° C., and then warmed to r.t. and stirred for 2 hrs. The reaction mixture was partitioned between H₂O and EtOAc and the phases were separated. The aqueous layer was extracted with EtOAc (2×) and the combined organic phases were washed with H₂O (1×), dried over anhydrous Na₂SO₄ and evaporated to dryness. The material was purified by chromatography, first on a 25 g silica gel column using a 0-30% EtOAc/cyclohexane gradient eluent and then on a 28 g NH silica gel column, using a 0-30% EtOAc/cyclohexane gradient eluent to afford the title compound (460 mg, 70%) as a white solid. ¹H NMR (400 MHz, DMSO-d₆) δ 8.02 (dd, J=9.79, 8.69 Hz, 2H) 7.89 (d, J=7.92 Hz, 1H) 7.44 (dd, J=9.02, 2.42 Hz, 1H) 7.21 (d, J=2.42 Hz, 1H) 5.81 (t, J=4.73 Hz, 1H) 2.87-3.07 (m, 2H) 1.86-2.16 (m, 4H) 1.65 (s, 9H). MS-ESI (m/z) calc'd for C₂₂H₂₂IN₄O₃ [M+H]⁺: 517.1. Found 517.1.

### Step 2: 8-((3-(Oxazol-5-yl)-1H-indazol-5-yl)oxy)-5,6,7,8-tetrahydroisoquinoline-3-carbonitrile, enantiomer 1 and 2

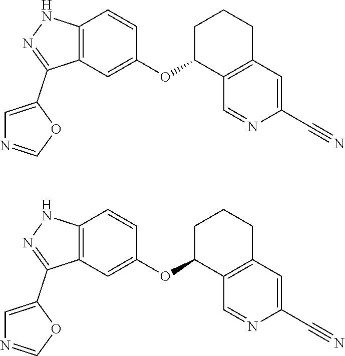 [SMILES: N#Cc1cc2c(cn1)[C@H](Oc1ccc3[nH]nc(-c4cnco4)c3c1)CCC2 ; N#Cc1cc2c(cn1)[C@@H](Oc1ccc3[nH]nc(-c4cnco4)c3c1)CCC2]

Prepared as described for 8-((3-(2-ethylpyridin-4-yl)-1H-indazol-5-yl)oxy)-5,6,7,8-tetrahydroquinoline-3-carbonitrile using tert-butyl 5-((3-cyano-5,6,7,8-tetrahydroisoquinolin-8-yl)oxy)-3-iodo-1H-indazole-1-carboxylate, in place of tert-butyl 5-[(3-cyano-5,6,7,8-tetrahydroquinolin-8-yl)oxy]-3-iodo-1H-indazole-1-carboxylate and 5-(4,4,5,5-tetramethyl-1,3,2-dioxaborolan-2-yl)-1,3-oxazole in place of 2-ethyl-4-(4,4,5,5-tetramethyl-1,3,2-dioxaborolan-2-yl)pyridine to afford the title compound (60 mg, 91%) as a racemic mixture. The mixture was purified by chiral separation using Method DK to afford 8-((3-(oxazol-5-yl)-1H-indazol-5-yl)oxy)-5,6,7,8-tetrahydroisoquinoline-3-carbonitrile, enantiomer 1 (21 mg, 32%) as a white solid. ¹H NMR (400 MHz, DMSO-d₆) δ 13.42 (br. s., 1H) 8.51 (s, 1H) 8.05 (d, J=8.14 Hz, 1H) 7.89 (d, J=7.92 Hz, 1H) 7.86 (s, 1H) 7.67 (d, J=2.20 Hz, 1H) 7.58 (d, J=9.02 Hz, 1H) 7.23 (dd, J=9.02, 2.42 Hz, 1H) 5.76 (t, J=4.62 Hz, 1H) 2.87-3.08 (m, 2H) 1.97-2.15 (m, 3H) 1.85-1.96 (m, 1H). MS-ESI (m/z) calc'd for C₂₀H₁₆N₅O₂ [M+H]⁺: 358.1. Found 358.1. A later eluting fraction was also isolated to afford 8-((3-(oxazol-5-yl)-1H-indazol-5-yl)oxy)-5,6,7,8-tetrahydroisoquinoline-3-carbonitrile, enantiomer 2 (19 mg, 29%). ¹H NMR (400 MHz, DMSO-d₆) δ 13.42 (br. s., 1H) 8.51 (s, 1H) 8.05 (d, J=7.92 Hz, 1H) 7.89 (d, J=7.92 Hz, 1H) 7.86 (s, 1H) 7.67 (d, J=2.20 Hz, 1H) 7.58 (d, J=9.02 Hz, 1H) 7.23 (dd, J=9.02, 2.42 Hz, 1H) 5.76 (t, J=4.51 Hz, 1H) 2.88-3.08 (m, 2H) 1.98-2.17 (m, 3H) 1.84-1.97 (m, 1H). MS-ESI (m/z) calc'd for C₂₀H₁₆N₅₀₂ [M+H]⁺: 358.1. Found 358.1.

### Example 123: 8-((3-(2-Methoxypyridin-4-yl)-1H-indazol-5-yl)oxy)-5,6,7,8-tetrahydroquinoline-3-carbonitrile, enantiomer 1 and 2

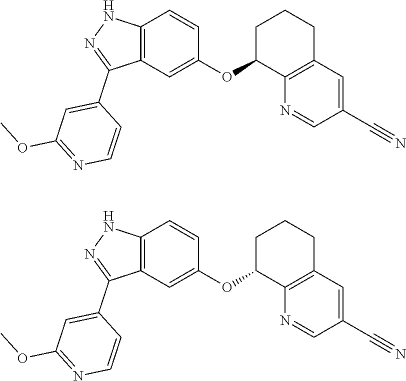 [SMILES: COc1cc(-c2n[nH]c3ccc(O[C@H]4CCCc5cc(C#N)cnc54)cc23)ccn1 ; COc1cc(-c2n[nH]c3ccc(O[C@@H]4CCCc5cc(C#N)cnc54)cc23)ccn1]

Prepared as described for 8-((3-(2-ethylpyridin-4-yl)-1H-indazol-5-yl)oxy)-5,6,7,8-tetrahydroquinoline-3-carbonitrile using (2-methoxypyridin-4-yl)boronic acid in place of 2-ethyl-4-(4,4,5,5-tetramethyl-1,3,2-dioxaborolan-2-yl)pyridine, to afford 8-{[3-(2-methoxypyridin-4-yl)-1H-indazol-5-yl]oxy}-5,6,7,8-tetrahydroquinoline-3-carbonitrile. The material was purified by chiral separation using Method DL to afford 8-((3-(2-methoxypyridin-4-yl)-1H-indazol-5-yl)oxy)-5,6,7,8-tetrahydroquinoline-3-carbonitrile, enantiomer 1 (16.3 mg, 26%) as a white solid. ¹H NMR (400 MHz, DMSO-d₆) δ 13.45 (br. s., 1H) 8.87 (d, J=1.98 Hz, 1H) 8.27 (d, J=5.72 Hz, 1H) 8.21 (d, J=1.98 Hz, 1H) 7.78 (d, J=2.20 Hz, 1H) 7.61 (dd, J=5.39, 1.43 Hz, 1H) 7.56 (d, J=9.02 Hz, 1H) 7.34 (d, J=0.66 Hz, 1H) 7.20 (dd, J=9.02, 2.20 Hz, 1H) 5.61-5.69 (m, 1H) 3.93 (s, 3H) 2.93-3.03 (m, 1H) 2.78-2.91 (m, 1H) 2.23-2.32 (m, 1H) 1.92-2.08 (m, 2H) 1.79-1.90 (m, 1H). MS-ESI (m/z) calc'd for C₂₃H₂₀N₅O₂ [M+H]⁺: 398.1. Found 398.1. A later eluting fraction was also isolated to afford 8-((3-(2-methoxypyridin-4-yl)-1H-indazol-5-yl)oxy)-5,6,7,8-tetrahydroquinoline-3-carbonitrile, enantiomer 2 (17.1 mg, 28%) as a white solid. ¹H NMR (400 MHz, DMSO-d₆) δ 13.46 (br. s., 1H) 8.87 (d, J=2.20 Hz, 1H) 8.27 (d, J=5.50 Hz, 1H) 8.21 (d, J=1.98 Hz, 1H) 7.78 (d, J=1.98 Hz, 1H) 7.61 (dd, J=5.39, 1.43 Hz, 1H) 7.56 (d, J=9.02 Hz, 1H) 7.34 (d, J=0.66 Hz, 1H) 7.20 (dd, J=9.02, 2.20 Hz, 1H) 5.62-5.68 (m, 1H) 3.93 (s, 3H) 2.92-3.06 (m, 1H) 2.76-2.90 (m, 1H) 2.21-2.31 (m, 1H) 1.91-2.09 (m, 2H) 1.78-1.90 (m, 1H). MS-ESI (m/z) calc'd for C₂₃H₂₀N₅O₂ [M+H]⁺: 398.1. Found 398.1.

### Example 124: 1-((3-Cyclopropyl-1H-indazol-5-yl)oxy)-2,3-dihydro-1H-indene-5-carbonitrile, enantiomer 1 and 2

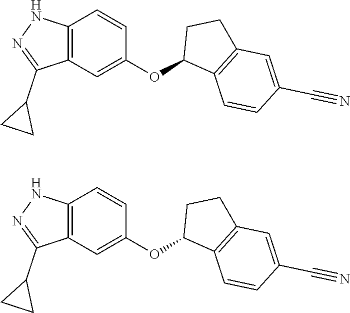 [SMILES: N#Cc1ccc2c(c1)CC[C@@H]2Oc1ccc2[nH]nc(C3CC3)c2c1 ; N#Cc1ccc2c(c1)CC[C@H]2Oc1ccc2[nH]nc(C3CC3)c2c1]

### Step 1: 1-Hydroxy-2,3-dihydro-1H-indene-5-carbonitrile

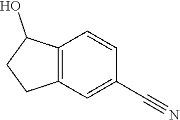 [SMILES: N#Cc1ccc2c(c1)CCC2O]

To a suspension of 2,3-dihydro-1-oxo-1H-indene-5-carbonitrile (1.57 g, 10.00 mmol) in MeOH (20.0 mL) was added sodium borohydride (756.6 mg, 20.00 mmol) and the mixture was stirred at 25° C. for 3 hrs. The solvent was evaporated and the residue was taken up in water and stirred for 1 hr. A solid formed that was collected by vacuum filtration to afford the title compound (1.23 g, 77) as a grey solid. ¹H NMR (400 MHz, DMSO-d₆) δ 7.68 (s, 1H), 7.67-7.62 (m, 1H), 7.50 (d, J=7.7 Hz, 1H), 5.49 (d, J=5.8 Hz, 1H), 5.08 (q, J=6.7 Hz, 1H), 2.94 (ddd, J=16.2, 8.8, 3.5 Hz, 1H), 2.75 (dt, J=16.3, 8.2 Hz, 1H), 2.37 (dddd, J=12.6, 8.0, 7.2, 3.5 Hz, 1H), 1.79 (dtd, J=12.7, 8.6, 7.0 Hz, 1H). MS-ESI (m/z) calc'd for C₁₀H₁₀NO [M+H]⁺: 160.0. Found 159.9.

### Step 2: tert-Butyl 5-((5-cyano-2,3-dihydro-1H-inden-1-yl)oxy)-3-iodo-1H-indazole-1-carboxylate

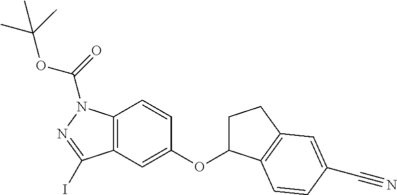 [SMILES: CC(C)(C)OC(=O)n1nc(I)c2cc(OC3CCc4cc(C#N)ccc43)ccc21]

To a solution of 1-hydroxy-2,3-dihydro-1H-indene-5-carbonitrile (159.1 mg, 1.00 mmol), tert-butyl 5-hydroxy-3-iodo-1H-indazole-1-carboxylate (360.1 mg, 1.00 mmol) and triphenylphosphine (262.3 mg, 1.00 mmol) was added diethyl azodicarboxylate (0.16 mL, 1.00 mmol). The mixture was stirred at 25° C. for 2 hrs. The solvent was then evaporate to dryness and the residue was purified by silica gel chromatography on a 25 g column using a 0-100% EtOAc/cyclohexane gradient eluent to afford the title compound (310 mg, 62%) as a white solid. ¹H NMR (400 MHz, DMSO-d₆) δ 7.99 (d, J=9.1 Hz, 1H), 7.84 (s, 1H), 7.71 (dd, J=7.9, 1.5 Hz, 1H), 7.58 (d, J=7.8 Hz, 1H), 7.38 (dd, J=9.1, 2.5 Hz, 1H), 7.11 (d, J=2.4 Hz, 1H), 6.10 (dd, J=6.7, 4.6 Hz, 1H), 3.11 (ddd, J=16.4, 8.6, 5.4 Hz, 1H), 3.03-2.92 (m, 1H), 2.66 (dddd, J=13.6, 8.4, 6.8, 5.3 Hz, 1H), 2.10 (dddd, J=13.4, 8.6, 6.0, 4.6 Hz, 1H), 1.64 (s, 9H). MS-ESI (m/z) calc'd for C₂₂H₂₁IN₃O₃ [M+H]⁺: 502.0. Found 502.0.

### Step 3: 1-((3-Cyclopropyl-1H-indazol-5-yl)oxy)-2,3-dihydro-1H-indene-5-carbonitrile, enantiomer 1 and 2

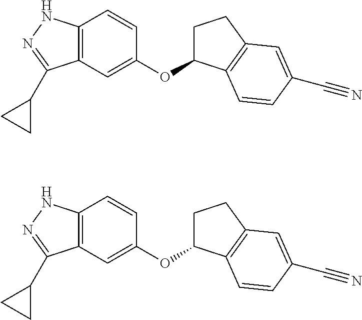 [SMILES: N#Cc1ccc2c(c1)CC[C@@H]2Oc1ccc2[nH]nc(C3CC3)c2c1 ; N#Cc1ccc2c(c1)CC[C@H]2Oc1ccc2[nH]nc(C3CC3)c2c1]

Prepared as described for 8-((3-(2-ethylpyridin-4-yl)-1H-indazol-5-yl)oxy)-5,6,7,8-tetrahydroquinoline-3-carbonitrile using cyclopropylboronic acid in place of 2-ethyl-4-(4,4,5,5-tetramethyl-1,3,2-dioxaborolan-2-yl)pyridine, and tert-butyl 5-((5-cyano-2,3-dihydro-1H-inden-1-yl)oxy)-3-iodoindazole-1-carboxylate in place of tert-butyl 5-((3-cyano-5,6,7,8-tetrahydroquinolin-8-yl)oxy)-3-iodo-1H-indazole-1-carboxylate, to afford 1-((3-cyclopropyl-1H-indazol-5-yl)oxy)-2,3-dihydro-1H-indene-5-carbonitrile. The material was purified by chiral separation using Method DM to afford 1-((3-cyclopropyl-1H-indazol-5-yl)oxy)-2,3-dihydro-1H-indene-5-carbonitrile, enantiomer 1 (7.9 mg, 4%) as a white solid. ¹H NMR (400 MHz, DMSO-d₆) δ 12.41 (s, 1H), 7.82 (s, 1H), 7.73-7.66 (m, 1H), 7.57 (d, J=7.9 Hz, 1H), 7.43-7.33 (m, 2H), 7.03 (dd, J=8.9, 2.3 Hz, 1H), 5.94 (dd, J=6.8, 4.9 Hz, 1H), 3.16-3.05 (m, 1H), 3.03-2.89 (m, 1H), 2.64 (dddd, J=13.5, 8.4, 6.7, 5.1 Hz, 1H), 2.30-2.21 (m, 1H), 2.11 (dddd, J=13.4, 8.6, 6.2, 4.8 Hz, 1H), 1.00-0.87 (m, 4H). MS-ESI (m/z) calc'd for C₂₀H₁₈N₃O [M+H]⁺: 316.1. Found 316.1. A later eluting fraction was also isolated to afford 1-((3-cyclopropyl-1H-indazol-5-yl)oxy)-2,3-dihydro-1H-indene-5-carbonitrile, enantiomer 2 (9.3 mg, 5%) as a white solid. ¹H NMR (400 MHz, DMSO-d₆) δ 12.41 (s, 1H), 7.82 (s, 1H), 7.70 (ddd, J=7.8, 1.6, 0.8 Hz, 1H), 7.57 (d, J=7.8 Hz, 1H), 7.44-7.32 (m, 2H), 7.03 (dd, J=8.9, 2.3 Hz, 1H), 5.94 (dd, J=6.7, 4.9 Hz, 1H), 3.16-3.04 (m, 1H), 3.01-2.87 (m, 1H), 2.64 (dddd, J=13.5, 8.4, 6.7, 5.1 Hz, 1H), 2.30-2.21 (m, 1H), 2.11 (dddd, J=13.5, 8.6, 6.2, 4.8 Hz, 1H), 1.02-0.86 (m, 4H). MS-ESI (m/z) calc'd for C₂₀H₁₈N₃O [M+H]⁺: 316.1. Found 316.1.

### Example 125: 8-((3-Bromo-1H-indazol-5-yl)amino)-5,6,7,8-tetrahydroquinoline-3-carbonitrile, enantiomer 1 and 2

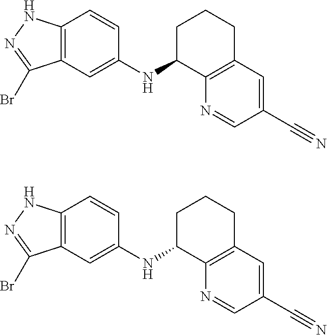 [SMILES: N#Cc1cnc2c(c1)CCC[C@@H]2Nc1ccc2[nH]nc(Br)c2c1 ; N#Cc1cnc2c(c1)CCC[C@H]2Nc1ccc2[nH]nc(Br)c2c1]

To a stirred solution of 3-bromo-1H-indazol-5-amine (184.73 mg, 0.87 mmol) and 8-oxo-5,6,7,8-tetrahydroquinoline-3-carbonitrile (100.0 mg, 0.580 mmol) in 1,4-dioxane (4.8 mL) was added 4-methylbenzenesulfonic acid hydrate (11.05 mg, 0.06 mmol) and the mixture was stirred at 100° C. for 3 hrs. The reaction was cooled to 40° C. and sodium triacetoxyborohydride (446.65 mg, 2.32 mmol) was added portionwise over 2 hrs. Then the mixture was left stirring at 40° C. overnight. The reaction mixture was partitioned between H₂O and EtOAc, the phases were separated, and the aqueous layer was extracted with EtOAc (2×). The combined organic phases were washed with water (1×), dried over anhydrous Na₂SO₄ and evaporated to dryness. The residue was purified by chromatography on a 25 g silica gel column using a 0-70% EtOAc/cyclohexane gradient eluent to give material of insufficient purity. The residue was then purified again by reversed phase chromatography on a 12 g C18 column using a 5-50% CH₃CN/H₂O gradient eluent in presence of 0.1% formic acid to afford 8-[(3-bromo-1H-indazol-5-yl)amino]-5,6,7,8-tetrahydroquinoline-3-carbonitrile (57 mg, 0.15 mmol, 27%) as a yellow solid. The enantiomers were separated by chiral chromatography using Method DN to afford the 8-((3-bromo-1H-indazol-5-yl)amino)-5,6,7,8-tetrahydroquinoline-3-carbonitrile, enantiomer 1 (16.9 mg, 8%) as an off-white solid. ¹H NMR (400 MHz, DMSO-d₆) δ 12.97 (s, 1H) 8.81 (d, J=1.98 Hz, 1H) 8.13 (d, J=1.98 Hz, 1H) 7.32 (d, J=9.02 Hz, 1H) 7.01 (dd, J=9.02, 2.20 Hz, 1H) 6.61 (d, J=1.76 Hz, 1H) 5.97 (d, J=7.26 Hz, 1H) 4.61-4.74 (m, 1H) 2.75-2.97 (m, 2H) 1.73-2.11 (m, 4H). MS-ESI (m/z) calc'd for C₁₇H₁₅BrN₅ [M+H]⁺: 368.0. Found 368.0. A later eluting fraction was also isolated to afford 8-((3-bromo-1H-indazol-5-yl)amino)-5,6,7,8-tetrahydroquinoline-3-carbonitrile, enantiomer 2 (17 mg, 8%) as an off-white solid. ¹H NMR (400 MHz, DMSO-d₆) δ 12.97 (s, 1H) 8.81 (d, J=1.98 Hz, 1H) 8.13 (d, J=1.98 Hz, 1H) 7.32 (d, J=9.02 Hz, 1H) 7.01 (dd, J=9.02, 2.20 Hz, 1H) 6.61 (d, J=1.76 Hz, 1H) 5.97 (d, J=7.26 Hz, 1H) 4.61-4.74 (m, 1H) 2.75-2.97 (m, 2H) 1.73-2.11 (m, 4H). MS-ESI (m/z) calc'd for C₁₇H₁₅BrN₅ [M+H]⁺: 368.0. Found 368.0.

### Example 126: 8-((3-Chloro-1H-indazol-5-yl)amino)-5,6,7,8-tetrahydroquinoline-3-carbonitrile, enantiomer 1 and 2

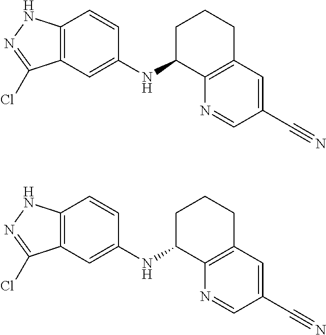 [SMILES: N#Cc1cnc2c(c1)CCC[C@@H]2Nc1ccc2[nH]nc(Cl)c2c1 ; N#Cc1cnc2c(c1)CCC[C@H]2Nc1ccc2[nH]nc(Cl)c2c1]

Prepared as described for 8-((3-bromo-1H-indazol-5-yl)amino)-5,6,7,8-tetrahydroquinoline-3-carbonitrile using 3-chloro-1H-indazol-5-amine in place of 3-bromo-1H-indazol-5-amine to afford 8-[(3-chloro-1H-indazol-5-yl)amino]-5,6,7,8-tetrahydroquinoline-3-carbonitrile (80.0 mg, 42%) which was purified by chiral separation using Method DO to afford 8-((3-chloro-1H-indazol-5-yl)amino)-5,6,7,8-tetrahydroquinoline-3-carbonitrile, enantiomer 1 (30.2 mg, 16%) as a yellow solid. ¹H NMR (400 MHz, DMSO-d₆) δ 12.81 (s, 1H) 8.81 (d, J=1.98 Hz, 1H) 8.13 (d, J=1.98 Hz, 1H) 7.31 (d, J=9.02 Hz, 1H) 7.01 (dd, J=9.13, 2.09 Hz, 1H) 6.68 (d, J=1.98 Hz, 1H) 5.96 (d, J=7.26 Hz, 1H) 4.62-4.74 (m, 1H) 2.74-3.01 (m, 2H) 1.76-2.13 (m, 4H). MS-ESI (m/z) calc'd for C₁₇H₁₅ClN₅ [M+H]⁺: 324.1. Found 324.1. A later eluting fraction was also isolated to afford 8-((3-chloro-1H-indazol-5-yl)amino)-5,6,7,8-tetrahydroquinoline-3-carbonitrile, enantiomer 2 (29.5 mg, 16%) as a yellow solid. ¹H NMR (400 MHz, DMSO-d₆) δ 12.82 (s, 1H) 8.81 (d, J=1.98 Hz, 1H) 8.13 (d, J=2.20 Hz, 1H) 7.31 (d, J=9.02 Hz, 1H) 7.01 (dd, J=9.02, 2.20 Hz, 1H) 6.68 (d, J=1.98 Hz, 1H) 5.96 (d, J=7.26 Hz, 1H) 4.60-4.77 (m, 1H) 2.75-3.01 (m, 2H) 1.76-2.12 (m, 4H). MS-ESI (m/z) calc'd for C₁₇H₁₅ClN₅ [M+H]⁺: 324.1. Found 324.1.

### Example 127: 1-((3-Iodo-1H-indazol-5-yl)amino)-4-methyl-2,3-dihydro-1H-indene-5-carbonitrile, enantiomer 1 and 2

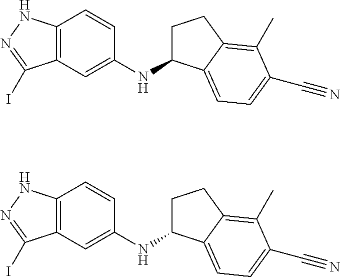 [SMILES: Cc1c(C#N)ccc2c1CC[C@@H]2Nc1ccc2[nH]nc(I)c2c1 ; Cc1c(C#N)ccc2c1CC[C@H]2Nc1ccc2[nH]nc(I)c2c1]

Prepared as described for 8-[(3-bromo-1H-indazol-5-yl)amino]-5,6,7,8-tetrahydroquinoline-3-carbonitrile, using 3-iodo-1H-indazol-5-amine in place of 3-bromo-1H-indazol-5-amine and 4-methyl-1-oxo-2,3-dihydro-1H-indene-5-carbonitrile in place of 8-oxo-5,6,7,8-tetrahydroquinoline-3-carbonitrile, to afford 1-((3-iodo-1H-indazol-5-yl)amino)-4-methyl-2,3-dihydro-1H-indene-5-carbonitrile (120 mg, 27%). The residue (30 mg, 0.072 mmol) was purified by chiral separation using Method DQ to afford 1-((3-iodo-1H-indazol-5-yl)amino)-4-methyl-2,3-dihydro-1H-indene-5-carbonitrile, enantiomer 1 (7.2 mg, 24%) as a white solid. ¹H NMR (400 MHz, DMSO-d₆) δ 13.10 (s, 1H), 7.59 (d, J=7.8 Hz, 1H), 7.32 (d, J=8.9 Hz, 1H), 7.27 (d, J=7.8 Hz, 1H), 7.00 (dd, J=8.9, 2.1 Hz, 1H), 6.49 (d, J=2.0 Hz, 1H), 5.98 (d, J=8.6 Hz, 1H), 5.11 (q, J=7.8 Hz, 1H), 3.08-2.93 (m, 1H), 2.91-2.76 (m, 1H), 2.64-2.54 (m, 1H), 2.43 (s, 3H), 1.92-1.77 (m, 1H). MS-ESI (m/z) calc'd for C₁₈H₁₆IN₄ [M+H]⁺: 415.0. Found 415.0. A later eluting fraction was also isolated to afford 1-((3-iodo-1H-indazol-5-yl)amino)-4-methyl-2,3-dihydro-1H-indene-5-carbonitrile, enantiomer 2 (7.3 mg, 25%) as a white solid. ¹H NMR (400 MHz, DMSO-d₆) δ 13.10 (s, 1H), 7.59 (d, J=7.8 Hz, 1H), 7.32 (d, J=8.9 Hz, 1H), 7.27 (d, J=7.8 Hz, 1H), 7.00 (dd, J=9.0, 2.1 Hz, 1H), 6.49 (d, J=2.1 Hz, 1H), 5.99 (d, J=8.6 Hz, 1H), 5.11 (q, J=7.7 Hz, 1H), 3.05-2.93 (m, 1H), 2.91-2.76 (m, 1H), 2.64-2.53 (m, 1H), 2.43 (s, 3H), 1.93-1.76 (m, 1H). MS-ESI (m/z) calc'd for C₁₈H₁₆IN₄ [M+H]⁺: 415.0. Found 415.0.

### Example 128: 1-((3-Iodo-1H-indazol-5-yl)amino)-2,3-dihydro-1H-indene-5-carbonitrile, enantiomer 1 and 2

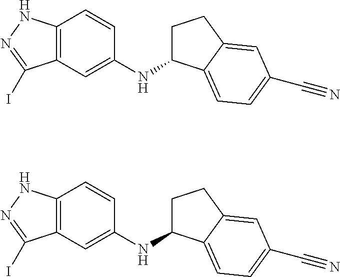 [SMILES: N#Cc1ccc2c(c1)CC[C@H]2Nc1ccc2[nH]nc(I)c2c1 ; N#Cc1ccc2c(c1)CC[C@@H]2Nc1ccc2[nH]nc(I)c2c1]

Prepared as described for 8-((3-bromo-1H-indazol-5-yl)amino)-5,6,7,8-tetrahydroquinoline-3-carbonitrile using 3-iodo-1H-indazol-5-amine in place of 3-bromo-1H-indazol-5-amine and 1-oxo-2,3-dihydro-1H-indene-5-carbonitrile in place of 8-oxo-5,6,7,8-tetrahydroquinoline-3-carbonitrile, to afford 1-((3-iodo-1H-indazol-5-yl)amino)-2,3-dihydro-1H-indene-5-carbonitrile (451 mg, 35%). 30 mg (0.072 mmol) of 1-[(3-iodo-1H-indazol-5-yl)amino]-2,3-dihydro-1H-indene-5-carbonitrile were subjected to chiral separation using Method DR to afford 1-((3-iodo-1H-indazol-5-yl)amino)-2,3-dihydro-1H-indene-5-carbonitrile, enantiomer 1 (8.6 mg, 28%) as a white solid. ¹H NMR (400 MHz, DMSO-d₆) δ 13.10 (s, 1H), 7.75 (s, 1H), 7.68-7.59 (m, 1H), 7.44 (d, J=7.8 Hz, 1H), 7.33 (d, J=8.9 Hz, 1H), 7.00 (dd, J=9.0, 2.1 Hz, 1H), 6.50 (d, J=2.1 Hz, 1H), 6.01 (d, J=8.7 Hz, 1H), 5.11 (q, J=7.9 Hz, 1H), 3.00 (ddd, J=12.2, 8.6, 4.3 Hz, 1H), 2.96-2.86 (m, 1H), 2.62-2.53 (m, 1H), 1.93-1.78 (m, 1H). MS-ESI (m/z) calc'd for C₁₇H₁₄IN₄ [M+H]⁺: 401.0. Found 401.0. A later eluting fraction was also isolated to afford 1-((3-iodo-1H-indazol-5-yl)amino)-2,3-dihydro-1H-indene-5-carbonitrile carbonitrile, enantiomer 2 (9.1 mg, 30%) as a white solid. ¹H NMR (400 MHz, DMSO-d₆) δ 13.10 (s, 1H), 7.76 (s, 1H), 7.64 (dd, J=7.8, 1.5 Hz, 1H), 7.45 (d, J=7.8 Hz, 1H), 7.34 (d, J=8.9 Hz, 1H), 7.01 (dd, J=9.0, 2.1 Hz, 1H), 6.51 (d, J=2.1 Hz, 1H), 6.01 (d, J=8.7 Hz, 1H), 5.12 (q, J=7.8 Hz, 1H), 3.02 (ddd, J=16.2, 8.6, 3.5 Hz, 1H), 2.97-2.87 (m, 1H), 2.63-2.53 (m, 1H), 1.94-1.79 (m, 1H). MS-ESI (m/z) calc'd for C₁₇H₁₄IN₄ [M+H]⁺: 401.0. Found 401.0.

### Example 129: 1-((3-Methyl-1H-indazol-5-yl)amino)-2,3-dihydro-1H-indene-4-carbonitrile, enantiomer 1 and 2

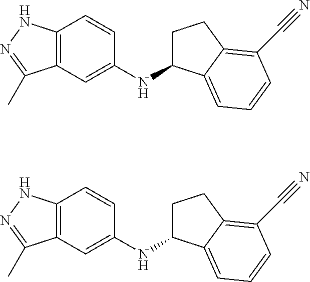 [SMILES: Cc1n[nH]c2ccc(N[C@H]3CCc4c(C#N)cccc43)cc12 ; Cc1n[nH]c2ccc(N[C@@H]3CCc4c(C#N)cccc43)cc12]

Prepared as described for 8-((3-bromo-1H-indazol-5-yl)amino)-5,6,7,8-tetrahydroquinoline-3-carbonitrile using 3-methyl-1H-indazol-5-amine in place of 3-bromo-1H-indazol-5-amine and 1-oxo-2,3-dihydro-1H-indene-4-carbonitrile in place of 8-oxo-5,6,7,8-tetrahydroquinoline-3-carbonitrile to afford 1-((3-methyl-1H-indazol-5-yl)amino)-2,3-dihydro-1H-indene-4-carbonitrile (56 mg, 44%). The mixture was subjected to chiral separation using Method DS to afford 1-((3-methyl-1H-indazol-5-yl)amino)-2,3-dihydro-1H-indene-4-carbonitrile, enantiomer 1 (22.5 mg, 17.5%) as a white solid. ¹H NMR (400 MHz, DMSO-d₆) δ 12.18 (s, 1H), 7.69 (d, J=7.6 Hz, 1H), 7.61 (d, J=7.6 Hz, 1H), 7.38 (t, J=7.6 Hz, 1H), 7.22 (d, J=8.8 Hz, 1H), 6.89 (dd, J=8.9, 2.1 Hz, 1H), 6.84 (d, J=2.0 Hz, 1H), 5.68 (d, J=9.0 Hz, 1H), 5.12 (q, J=7.7 Hz, 1H), 3.13 (ddd, J=16.5, 8.7, 3.9 Hz, 1H), 3.01 (dt, J=16.4, 8.1 Hz, 1H), 2.69-2.57 (m, 1H), 2.39 (s, 3H), 1.90 (dq, J=12.6, 8.1 Hz, 1H). MS-ESI (m/z) calc'd for C₁₈H₁₇N₄ [M+H]⁺: 289.1. Found 289.1. A later eluting fraction was also isolated to afford 1-((3-methyl-1H-indazol-5-yl)amino)-2,3-dihydro-1H-indene-4-carbonitrile, enantiomer 2 (23.1 mg, 18%) as a white solid. ¹H NMR (400 MHz, DMSO-d₆) δ 12.18 (s, 1H), 7.69 (d, J=7.6 Hz, 1H), 7.61 (d, J=7.6 Hz, 1H), 7.38 (t, J=7.7 Hz, 1H), 7.22 (d, J=8.8 Hz, 1H), 6.89 (dd, J=8.8, 2.1 Hz, 1H), 6.83 (d, J=2.0 Hz, 1H), 5.69 (d, J=9.0 Hz, 1H), 5.12 (q, J=7.6 Hz, 1H), 3.13 (ddd, J=16.5, 8.7, 3.9 Hz, 1H), 3.01 (dt, J=16.4, 8.1 Hz, 1H), 2.69-2.57 (m, 1H), 2.39 (s, 3H), 1.90 (dq, J=12.6, 8.2 Hz, 1H). MS-ESI (m/z) calc'd for C₁₈H₁₇N₄ [M+H]⁺: 289.1. Found 289.1.

### Example 130: 3-((3-Methyl-1H-indazol-5-yl)amino)-2,3-dihydro-1H-indene-5-carbonitrile, enantiomer 1 and 2

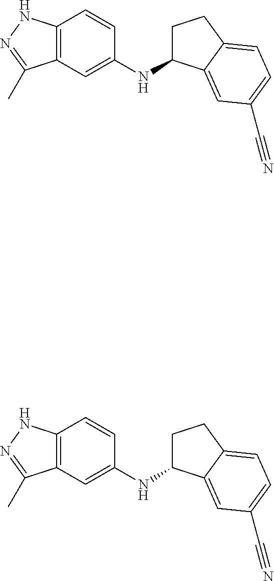 [SMILES: Cc1n[nH]c2ccc(N[C@H]3CCc4ccc(C#N)cc43)cc12 ; Cc1n[nH]c2ccc(N[C@@H]3CCc4ccc(C#N)cc43)cc12]

Prepared as described for 8-((3-bromo-1H-indazol-5-yl)amino]) 5,6,7,8-tetrahydroquinoline-3-carbonitrile using 3-methyl-1H-indazol-5-amine in place of 3-bromo-1H-indazol-5-amine and 3-oxo-2,3-dihydro-1H-indene-5-carbonitrile in place of 8-oxo-5,6,7,8-tetrahydroquinoline-3-carbonitrile to afford 3-((3-methyl-1H-indazol-5-yl)amino)-2,3-dihydro-1H-indene-5-carbonitrile (56 mg, 44%). The racemic mixture was subjected to chiral separation using Method DT to afford 3-((3-methyl-1H-indazol-5-yl)amino)-2,3-dihydro-1H-indene-5-carbonitrile, enantiomer 1 (22 mg, 17%) as a beige solid. ¹H NMR (400 MHz, DMSO-d₆) δ 12.19 (s, 1H), 7.69 (dd, J=7.6, 1.6 Hz, 1H), 7.66 (s, 1H), 7.50 (d, J=7.8 Hz, 1H), 7.23 (d, J=8.8 Hz, 1H), 6.90 (dd, J=8.9, 2.1 Hz, 1H), 6.83 (d, J=2.0 Hz, 1H), 5.69 (d, J=8.6 Hz, 1H), 5.06 (q, J=7.6 Hz, 1H), 3.05 (ddd, J=16.8, 8.6, 3.7 Hz, 1H), 2.93 (dt, J=16.6, 8.2 Hz, 1H), 2.60 (dtd, J=11.7, 7.6, 3.8 Hz, 1H), 2.40 (s, 3H), 1.83 (dq, J=12.5, 8.4 Hz, 1H). MS-ESI (m/z) calc'd for C₁₈H₁₇N₄ [M+H]⁺: 289.1. Found 289.1. A later eluting fraction was also isolated to afford 3-((3-methyl-1H-indazol-5-yl)amino)-2,3-dihydro-1H-indene-5-carbonitrile, enantiomer 2 (25 mg, 19.5%) as a beige solid. ¹H NMR (400 MHz, DMSO-d₆) δ 12.19 (s, 1H), 7.69 (dd, J=7.7, 1.6 Hz, 1H), 7.66 (s, 1H), 7.50 (d, J=7.7 Hz, 1H), 7.23 (d, J=8.8 Hz, 1H), 6.90 (dd, J=8.8, 2.1 Hz, 1H), 6.83 (d, J=2.0 Hz, 1H), 5.69 (d, J=8.6 Hz, 1H), 5.06 (q, J=7.6 Hz, 1H), 3.05 (ddd, J=16.7, 8.7, 3.7 Hz, 1H), 2.93 (dt, J=16.6, 8.1 Hz, 1H), 2.60 (dtd, J=11.7, 7.6, 3.8 Hz, 1H), 2.40 (s, 3H), 1.83 (dq, J=12.5, 8.3 Hz, 1H). MS-ESI (m/z) calc'd for C₁₈H₁₇N₄ [M+H]⁺: 289.1. Found 289.2.

### Example 131: 7-Methyl-1-((3-(oxazol-5-yl)-1H-indazol-5-yl)amino)-2,3-dihydro-1H-indene-5-carbonitrile, enantiomer 1 and 2

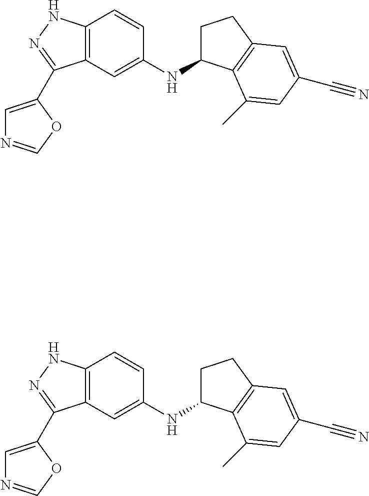 [SMILES: Cc1cc(C#N)cc2c1[C@@H](Nc1ccc3[nH]nc(-c4cnco4)c3c1)CC2 ; Cc1cc(C#N)cc2c1[C@H](Nc1ccc3[nH]nc(-c4cnco4)c3c1)CC2]

### Step 1: 5-Bromo-7-methyl-2,3-dihydro-1H-inden-1-ol

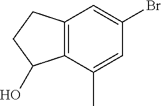

To a solution of 5-bromo-7-methyl-2,3-dihydro-1H-inden-1-one (250.0 mg, 1.11 mmol) in MeOH (15.0 mL) was added sodium borohydride (84.0 mg, 2.22 mmol) and the mixture was stirred at 25° C. for 2 hrs. Water was added and the reaction was extracted DCM (3×). The combined organic layers were dried over Na₂SO₄ and evaporated to afford the title compound (244 mg, 96%) as a white solid. ¹H NMR (400 MHz, DMSO-d₆) δ 7.23 (d, J=1.7 Hz, 1H), 7.16 (dq, J=1.6, 0.8 Hz, 1H), 5.06 (td, J=6.4, 3.0 Hz, 1H), 4.99 (d, J=6.4 Hz, 1H), 2.99 (dt, J=15.8, 7.8 Hz, 1H), 2.77-2.62 (m, 1H), 2.28-2.16 (m, 1H), 1.85 (dddd, J=13.5, 8.4, 4.3, 3.1 Hz, 1H). MS-ESI (m/z) calc'd for C₁₀H₁₂BrO [M+H]⁺: 227.0. Found 208.9 [M−H₂O]⁺.

### Step 2: 1-Hydroxy-7-methyl-2,3-dihydro-1H-indene-5-carbonitrile

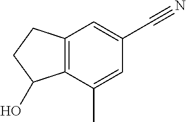

To a solution of 5-bromo-7-methyl-2,3-dihydro-1H-inden-1-ol (160.0 mg, 0.70 mmol) in 1,4-dioxane (3.0 mL) and H₂O (0.5 mL) were added 0.1 M potassium hexacyanoferrate (7.0 mL, 0.70 mmol) and KOAc (138.3 mg, 1.41 mmol). Then XPhos (33.6 mg, 0.07 mmol) and XPhos Pd G3 (59.6 mg, 0.07 mmol) were added and the mixture was stirred at 120° C. for 4 hrs. The reaction mixture was partitioned between H₂O and EtOAc and the phases were separated. The aqueous layer was extracted with EtOAc (2×) and the combined organic phases were washed with brine (1×), dried over anhydrous Na₂SO₄, and evaporated to dryness. The residue was purified by chromatography on a 10 g SiO₂ column using a 0-50% EtOAc/cyclohexane gradient eluent to afford the title compound (63 mg, 52%) as a yellow solid. ¹H NMR (400 MHz, DMSO-d₆) δ 7.51 (s, 1H), 7.44 (s, 1H), 5.19 (d, J=6.6 Hz, 1H), 5.15 (td, J=6.6, 3.4 Hz, 1H), 3.02 (dt, J=15.7, 7.7 Hz, 1H), 2.75 (ddd, J=16.3, 8.8, 4.6 Hz, 1H), 2.38 (s, 3H), 2.27 (ddt, J=13.4, 8.9, 6.8 Hz, 1H), 1.89 (dddd, J=13.3, 8.3, 4.6, 3.3 Hz, 1H). MS-ESI (m/z) calc'd for C₁₁H₁₂NO [M+H]⁺: 174.0. Found 173.9.

### Step 3: N-(5-Cyano-7-methyl-2,3-dihydro-1H-inden-1-yl)-2-nitro-N-(3-(oxazol-5-yl)-1-((2-(trimethylsilyl)ethoxy)methyl)-1H-indazol-5-yl)benzenesulfonamide

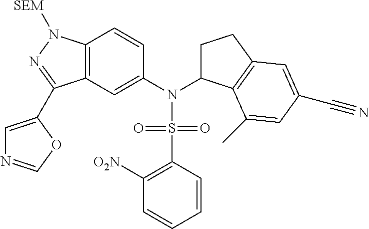 [SMILES: Cc1cc(C#N)cc2c1C(N(c1ccc3c(c1)c(-c1cnco1)nn3COCC[Si](C)(C)C)S(=O)(=O)c1ccccc1[N+](=O)[O-])CC2]

To a solution of 1-hydroxy-7-methyl-2,3-dihydro-1H-indene-5-carbonitrile (63.0 mg, 0.36 mmol), 2-nitro-N-[3-(1,3-oxazol-5-yl)-1-(2-trimethylsilylethoxymethyl)indazol-5-yl]benzenesulfonamide (187.5 mg, 0.36 mmol) and triphenylphosphine (190.8 mg, 0.73 mmol) in THF (3.6 mL) was added diethyl azodicarboxylate (114.5 uL, 0.73 mmol) dropwise and the mixture was stirred at 25° C. for 15 hrs. The solvent was evaporated to dryness and the residue was purified by chromatography on an 11 g NH column using a 0-50% EtOAc/cyclohexane gradient eluent to afford the title compound (244 mg, 100%) as an orange solid. ¹H NMR (400 MHz, DMSO-d₆) δ 8.57 (d, J=9.6 Hz, 1H), 8.14-7.07 (m, 10H), 6.04 (d, J=7.1 Hz, 1H), 5.76 (d, J=4.4 Hz, 2H), 3.67-3.45 (m, 3H), 2.54 (s, 3H), 2.44 (d, J=5.0 Hz, 2H), 1.95-1.74 (m, 1H), 0.89-0.71 (m, 2H), −0.16 (d, J=7.6 Hz, 9H). MS-ESI (m/z) calc'd for C₃₃H₃₅N₆O₆SiS [M+H]⁺: 671.2. Found 671.0.

### Step 4: N-(5-Cyano-7-methyl-2,3-dihydro-1H-inden-1-yl)-2-nitro-N-(3-(oxazol-5-yl)-1H-indazol-5-yl)benzenesulfonamide

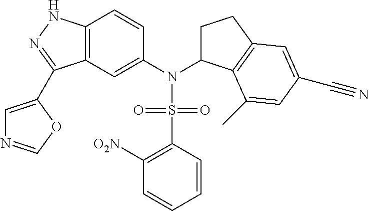 [SMILES: Cc1cc(C#N)cc2c1C(N(c1ccc3[nH]nc(-c4cnco4)c3c1)S(=O)(=O)c1ccccc1[N+](=O)[O-])CC2]

A solution of N-(5-cyano-7-methyl-2,3-dihydro-1H-inden-1-yl)-2-nitro-N-(3-(oxazol-5-yl)-1-((2-(trimethylsilyl)ethoxy)methyl)-1H-indazol-5-yl)benzenesulfonamide (244.0 mg, 0.36 mmol) in DCM (4 mL) and trifluoroacetic acid (1.0 mL) was stirred at 25° C. for 24 hrs. The solvent was evaporated to afford the title compound (196 mg, 99%) as an orange oil which was used without further purification. MS-ESI (m/z) calc'd for C₂₇H₂IN₆₀₅S [M+H]⁺: 541.1. Found 541.0.

### Step 5: 7-Methyl-1-((3-(oxazol-5-yl)-1H-indazol-5-yl)amino)-2,3-dihydro-1H-indene-5-carbonitrile, enantiomer 1 and 2

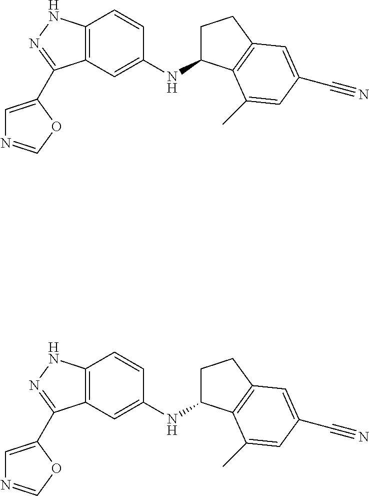 [SMILES: Cc1cc(C#N)cc2c1[C@@H](Nc1ccc3[nH]nc(-c4cnco4)c3c1)CC2 ; Cc1cc(C#N)cc2c1[C@H](Nc1ccc3[nH]nc(-c4cnco4)c3c1)CC2]

To a solution of N-(5-cyano-7-methyl-2,3-dihydro-1H-inden-1-yl)-2-nitro-N-[3-(1,3-oxazol-5-yl)-1H-indazol-5-yl]benzene-1-sulfonamide (196.0 mg, 0.36 mmol) in DMF (3.0 mL) were added K₂CO₃ (200.4 mg, 1.45 mmol) and benzenethiol (111.3 uL, 1.09 mmol) and the mixture was stirred at 25° C. for 2 hrs. Water was added and the mixture was extracted with EtOAc. The organic layer was evaporated to dryness. The material was purified by chromatography on an 11 g NH column using a 0-100% EtOAc/cyclohexane gradient eluent to afford 7-methyl-1-{[3-(1,3-oxazol-5-yl)-1H-indazol-5-yl]amino}-2,3-dihydro-1H-indene-5-carbonitrile (23.0 mg, 18%). The racemic mixture was subjected to chiral separation using Method DU to afford 7-methyl-1-((3-(oxazol-5-yl)-1H-indazol-5-yl)amino)-2,3-dihydro-1H-indene-5-carbonitrile, enantiomer 1 (6.0 mg, 5%) as a yellow solid. ¹H NMR (400 MHz, DMSO-d₆) δ p 13.08 (s, 1H), 8.47 (s, 1H), 7.65 (s, 1H), 7.61 (s, 1H), 7.51 (s, 1H), 7.36 (d, J=8.9 Hz, 1H), 6.99 (d, J=2.0 Hz, 1H), 6.96 (dd, J=8.9, 2.1 Hz, 1H), 5.83 (d, J=8.2 Hz, 1H), 5.17 (td, J=7.5, 2.7 Hz, 1H), 3.10 (dt, J=16.5, 8.3 Hz, 1H), 2.90 (ddd, J=16.5, 8.9, 3.4 Hz, 1H), 2.44-2.34 (m, 1H), 2.32 (s, 3H), 2.03 (ddt, J=12.8, 8.4, 3.1 Hz, 1H). MS-ESI (m/z) calc'd for C₂₁H₁₈N₅O [M+H]⁺: 356.1. Found 356.1. A later eluting fraction was also isolated to afford 7-methyl-1-((3-(oxazol-5-yl)-1H-indazol-5-yl)amino)-2,3-dihydro-1H-indene-5-carbonitrile, enantiomer 2 (6.0 mg, 5%) as a yellow solid. ¹H NMR (400 MHz, DMSO-d₆) δ 13.08 (s, 1H), 8.47 (s, 1H), 7.65 (s, 1H), 7.61 (s, 1H), 7.51 (s, 1H), 7.36 (d, J=8.9 Hz, 1H), 6.99 (d, J=2.0 Hz, 1H), 6.96 (dd, J=8.9, 2.1 Hz, 1H), 5.83 (d, J=8.2 Hz, 1H), 5.23-5.10 (m, 1H), 3.10 (dt, J=16.5, 8.3 Hz, 1H), 2.90 (ddd, J=16.5, 8.8, 3.4 Hz, 1H), 2.44-2.34 (m, 1H), 2.32 (s, 3H), 2.03 (ddt, J=13.0, 8.4, 3.2 Hz, 1H). MS-ESI (m/z) calc'd for C₂₁H₁₈N₅O [M+H]⁺: 356.1. Found 356.1.

### Example 132: 5-((5-Cyano-7-fluoro-2,3-dihydro-1H-inden-1-yl)amino)-3-(oxazol-5-yl)-1H-indazol-2-ium, enantiomer 1 and 2

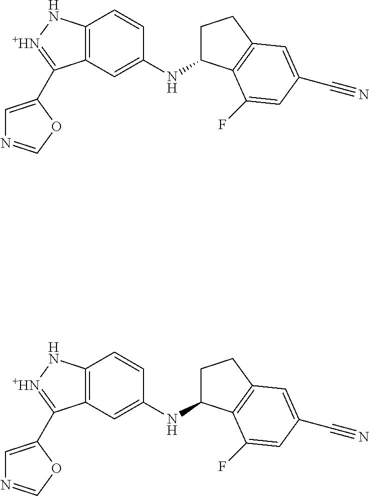 [SMILES: N#Cc1cc(F)c2c(c1)CC[C@H]2Nc1ccc2[nH][nH+]c(-c3cnco3)c2c1 ; N#Cc1cc(F)c2c(c1)CC[C@@H]2Nc1ccc2[nH][nH+]c(-c3cnco3)c2c1]

### Step 1: 5-Bromo-7-methyl-2,3-dihydro-1H-inden-1-ol

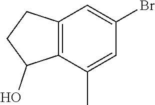

To a solution of 5-bromo-7-fluoro-2,3-dihydro-1H-inden-1-one (1.15 g, 5.00 mmol) in MeOH (10.0 mL) was added sodium borohydride (283.7 mg, 7.50 mmol) and the mixture was stirred at 25° C. for 1 hr. The solvent was evaporated and the residue was taken up in H₂O and extracted with Et₂O (3×). The combined organic layers were passed through a phase separator and evaporated to afford the title compound (1.15 g, 100%) as a dark oil. ¹H NMR (400 MHz, DMSO-d₆) δ 7.33-7.30 (m, 1H), 7.27 (ddt, J=8.7, 1.6, 0.8 Hz, 1H), 5.32 (d, J=6.3 Hz, 1H), 5.19 (td, J=6.5, 3.3 Hz, 1H), 3.04 (dddt, J=16.4, 8.1, 7.0, 1.1 Hz, 1H), 2.77 (dddt, J=16.5, 8.7, 4.5, 0.9 Hz, 1H), 2.27 (ddt, J=13.6, 8.7, 6.8 Hz, 1H), 1.88 (dddd, J=13.2, 8.1, 4.5, 3.3 Hz, 1H). MS-ESI (m/z) calc'd for C₉H₉BrFO [M+H]⁺: 230.9, 232.9. Found 212.9, 214.9 [M−H₂O]⁺.

### Step 2: ((5-Bromo-7-fluoro-2,3-dihydro-1H-inden-1-yl)oxy)(tert-butyl)dimethylsilane

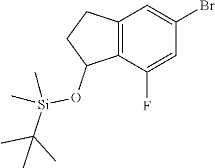 [SMILES: CC(C)(C)[Si](C)(C)OC1CCc2cc(Br)cc(F)c21]

To a solution of 5-bromo-7-fluoro-2,3-dihydro-1H-inden-1-ol (1.16 g, 5.00 mmol) in DMF (10.0 mL) was added tert-butyl-chloro-dimethylsilane (0.83 g, 5.50 mmol) and imidazole (0.68 g, 10.00 mmol). The mixture was stirred at 25° C. for 20 hrs. Water was added and the mixture was extracted with Et₂O (3×). The combined organic layers were washed with water (2×), dried over Na₂SO₄, and evaporated to dryness. The residue was purified by chromatography on a 25 g SiO₂ column using a 0-15% EtOAc/cyclohexane gradient eluent to afford the title compound (1.44 g, 83%) as a clear oil. ¹H NMR (400 MHz, DMSO-d₆) δ 7.34-7.32 (m, 1H), 7.32-7.28 (m, 1H), 5.41 (dd, J=6.7, 3.7 Hz, 1H), 3.09-2.98 (m, 1H), 2.85-2.73 (m, 1H), 2.35 (ddt, J=13.1, 8.6, 6.4 Hz, 1H), 1.88 (dddd, J=13.5, 8.7, 5.2, 3.8 Hz, 1H), 0.85 (s, 9H), 0.11 (s, 3H), 0.09 (d, J=0.7 Hz, 3H). MS-ESI (m/z) calc'd for C₁₅H₂₃BrFOSi [M+H]⁺: 345.0. Found no ionization.

### Step 3: 1-((tert-Butyldimethylsilyl)oxy)-7-fluoro-2,3-dihydro-1H-indene-5-carbonitrile

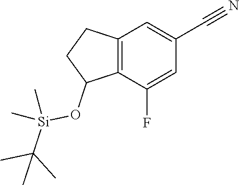 [SMILES: CC(C)(C)[Si](C)(C)OC1CCc2cc(C#N)cc(F)c21]

To a suspension of (5-bromo-7-fluoro-2,3-dihydro-1H-inden-1-yl)oxy-tert-butyl-dimethylsilane (1.04 g, 3.00 mmol), XPhos Pd G3 (253.94 mg, 0.300 mmol) and KOAc (588.8 mg, 6.00 mmol) was added 0.1 M potassium ferrocyanide (30.0 mL, 3.00 mmol) and the mixture was stirred at 100° C. for 2 hrs. The mixture was then diluted with H₂O and EtOAc. The biphasic mixture was filtered through Celite, then the two layers were separated and the organic phase was dried over Na₂SO₄ and evaporated to dryness. The residue was purified by chromatography on a 10 g SiO₂ column using a 0-10% EtOAc/cyclohexane gradient eluent to afford the title compound (754 mg, 86%) as an orange solid. ¹H NMR (400 MHz, DMSO-d₆) δ 7.65-7.59 (m, 2H), 5.51 (dd, J=6.8, 4.3 Hz, 1H), 3.07 (ddd, J=15.3, 8.6, 5.8 Hz, 1H), 2.91-2.77 (m, 1H), 2.41 (dddd, J=12.8, 8.5, 6.8, 5.8 Hz, 1H), 1.91 (dddd, J=13.0, 8.5, 5.8, 4.3 Hz, 1H), 0.86 (s, 9H), 0.13 (s, 3H), 0.11 (s, 3H). MS-ESI (m/z) calc'd for C₁₆H₂₃FNOSi [M+H]⁺: 292.1. Found 292.2.

### Step 4: 7-Fluoro-1-hydroxy-2,3-dihydro-1H-indene-5-carbonitrile

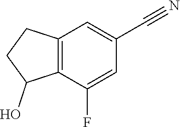 [SMILES: N#Cc1cc(F)c2c(c1)CCC2O]

To a solution of 1-[tert-butyl(dimethyl)silyl]oxy-7-fluoro-2,3-dihydro-1H-indene-5-carbonitrile (291.4 mg, 1.00 mmol) in THF (5.0 mL) was added 1.0 M tetrabutylammonium fluoride (1.0 mL, 1.00 mmol) dropwise at 0° C. and the mixture was stirred at 0° C. for 2 hrs. Water was added to quench the reaction and the THF was evaporated from the mixture under reduced pressure at room temperature to give an aqueous suspension. The suspension was extracted with EtOAc. The organic layer was passed through a phase separator and evaporated to dryness. The residue was purified by chromatography on a 10 g SiO₂ column using a 0-50% EtOAc/cyclohexane gradient to afford the title compound (85 mg, 48%) as an orange solid. ¹H NMR (400 MHz, DMSO-d₆) δ 7.65-7.56 (m, 2H), 5.50 (d, J=6.4 Hz, 1H), 5.29 (td, J=6.7, 3.8 Hz, 1H), 3.07 (dt, J=15.6, 7.2 Hz, 1H), 2.82 (ddd, J=16.5, 8.6, 4.9 Hz, 1H), 2.32 (ddt, J=13.5, 8.7, 6.7 Hz, 1H), 1.92 (dddd, J=13.3, 8.5, 4.9, 3.8 Hz, 1H). MS-ESI (m/z) calc'd for C₁₀H₉FNO [M+H]⁺: 178.0. Found 177.9.

### Step 5: N-(5-Cyano-7-fluoro-2,3-dihydro-1H-inden-1-yl)-2-nitro-N-(3-(oxazol-5-yl)-1-((2-(trimethylsilyl)ethoxy)methyl)-1H-indazol-5-yl)benzenesulfonamide

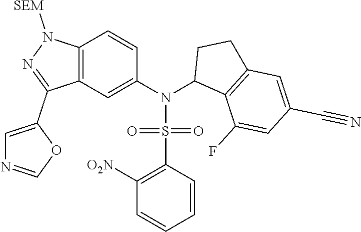 [SMILES: C[Si](C)(C)CCOCn1nc(-c2cnco2)c2cc(N(C3CCc4cc(C#N)cc(F)c43)S(=O)(=O)c3ccccc3[N+](=O)[O-])ccc21]

To a solution of 7-fluoro-1-hydroxy-2,3-dihydro-1H-indene-5-carbonitrile (85.0 mg, 0.48 mmol), 2-nitro-N-[3-(1,3-oxazol-5-yl)-1-(2-trimethylsilylethoxymethyl)indazol-5-yl]benzenesulfonamide (247.3 mg, 0.48 mmol) and triphenylphosphine (125.8 mg, 0.48 mmol) in THF (5.0 mL), was added diethyl azodicarboxylate (0.08 mL, 0.48 mmol) and the mixture was stirred at 25° C. for 15 hrs. The solvent was then evaporated and the residue was purified by chromatography on an 11 g NH column using a 0-50% EtOAc/cyclohexane gradient eluent to afford the title compound (153 mg, 47%) as an orange solid. ¹H NMR (400 MHz, DMSO-d₆) δ 8.57 (s, 1H), 8.10-8.03 (m, 1H), 7.96 (ddd, J=8.2, 6.0, 2.7 Hz, 1H), 7.86-7.71 (m, 2H), 7.69 (d, J=9.0 Hz, 1H), 7.64-7.54 (m, 2H), 7.40 (s, 1H), 7.32 (s, 1H), 6.93 (d, J=8.8 Hz, 1H), 6.19 (d, J=7.9 Hz, 1H), 5.75 (s, 2H), 3.52 (t, J=7.8 Hz, 2H), 3.07 (dt, J=15.8, 7.5 Hz, 1H), 2.82 (ddd, J=16.5, 8.5, 4.9 Hz, 1H), 2.37-2.24 (m, 1H), 1.99-1.84 (m, 1H), 0.77 (td, J=7.5, 2.0 Hz, 2H), −0.18 (s, 9H). MS-ESI (m/z) calc'd for C₁₀H₉FNO [M+H]⁺: 675.1. Found 675.1.

### Step 6: N-(5-Cyano-7-fluoro-2,3-dihydro-1H-inden-1-yl)-2-nitro-N-(3-(oxazol-5-yl)-1H-indazol-5-yl)benzenesulfonamide

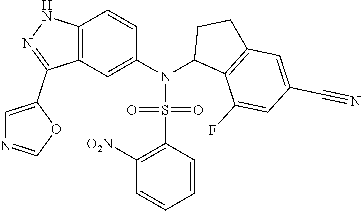 [SMILES: N#Cc1cc(F)c2c(c1)CCC2N(c1ccc2[nH]nc(-c3cnco3)c2c1)S(=O)(=O)c1ccccc1[N+](=O)[O-]]

A solution of N-(5-cyano-7-fluoro-2,3-dihydro-1H-inden-1-yl)-2-nitro-N-[3-(1,3-oxazol-5-yl)-1-(2-trimethylsilylethoxymethyl)indazol-5-yl]benzenesulfonamide (153.0 mg, 0.23 mmol) in DCM (5 mL) and trifluoroacetic acid (1 mL) was stirred at 25° C. for 24 hrs. The solvent was evaporated to afford the title compound (123 mg, 100%) as an orange oil which was used without further purification. MS-ESI (m/z) calc'd for C₁₀H₉FNO [M+H]⁺: 545.1. Found 545.0.

### Step 7: 5-((5-Cyano-7-fluoro-2,3-dihydro-1H-inden-1-yl)amino)-3-(oxazol-5-yl)-1H-indazol-2-ium, enantiomer 1 and 2

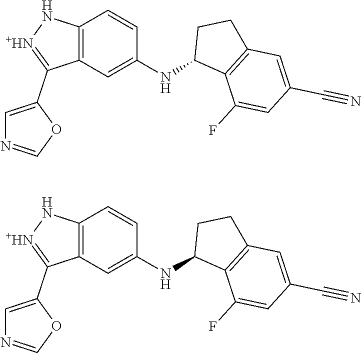 [SMILES: N#Cc1cc(F)c2c(c1)CC[C@H]2Nc1ccc2[nH][nH+]c(-c3cnco3)c2c1 ; N#Cc1cc(F)c2c(c1)CC[C@@H]2Nc1ccc2[nH][nH+]c(-c3cnco3)c2c1]

Prepared as described for 7-methyl-1-([3-(1,3-oxazol-5-yl)-1H-indazol-5-yl]amin))-2,3-dihydro-1H-indene-5-carbonitrile using N-(5-cyano-7-fluoro-2,3-dihydro-1H-inden-1-yl)-4-nitro-N-[3-(1,3-oxazol-5-yl)-1H-indazol-5-yl]benzene-1-sulfonamide in place of N-(5-cyano-7-methyl-2,3-dihydro-1H-inden-1-yl)-2-nitro-N((3-(1,3-oxazol-5-yl)-1H-indazol-5-yl)benzene-1-sulfonamide to afford 7-fluoro-1-((3-(1,3-oxazol-5-yl)-1H-indazol-5-yl)amino}-2,3-dihydro-1H-indene-5-carbonitrile (32.0 mg, 39%), which was subjected to chiral separation using Method DV to afford 5-((5-cyano-7-fluoro-2,3-dihydro-1H-inden-1-yl)amino)-3-(oxazol-5-yl)-1H-indazol-2-ium, enantiomer 1 (13.9 mg, 17%) as a yellow solid. ¹H NMR (400 MHz, DMSO-d₆) δ 13.09 (s, 1H), 8.46 (s, 1H), 7.68 (s, 1H), 7.66 (s, 1H), 7.63 (d, J=9.0 Hz, 1H), 7.36 (d, J=9.0 Hz, 1H), 7.06-7.01 (m, 1H), 6.93 (dd, J=9.0, 2.1 Hz, 1H), 6.02 (d, J=8.6 Hz, 1H), 5.46-5.30 (m, 1H), 3.15 (dt, J=16.0, 7.7 Hz, 1H), 3.02-2.87 (m, 1H), 2.56-2.43 (m, 1H), 2.04 (ddt, J=13.1, 8.7, 4.7 Hz, 1H). MS-ESI (m/z) calc'd for C₂₀H₁₅FN₅O [M+H]⁺: 360.1. Found 360.1. A later eluting fraction was also isolated to afford 5-((5-cyano-7-fluoro-2,3-dihydro-1H-inden-1-yl)amino)-3-(oxazol-5-yl)-1H-indazol-2-ium, enantiomer 2 (11.2 mg, 14%) as a yellow solid. ¹H NMR (400 MHz, DMSO-d₆) δ 13.09 (s, 1H), 8.46 (s, 1H), 7.68 (d, J=1.2 Hz, 1H), 7.66 (s, 1H), 7.63 (dd, J=8.9, 1.2 Hz, 1H), 7.36 (d, J=9.0 Hz, 1H), 7.03 (d, J=2.0 Hz, 1H), 6.93 (dd, J=9.0, 2.1 Hz, 1H), 6.02 (d, J=8.5 Hz, 1H), 5.37 (td, J=7.8, 4.0 Hz, 1H), 3.15 (dt, J=15.8, 7.7 Hz, 1H), 3.02-2.88 (m, 1H), 2.54-2.46 (m, 1H), 2.04 (ddt, J=13.0, 8.8, 4.6 Hz, 1H). MS-ESI (m/z) calc'd for C₂₀H₁₅FN₅O [M+H]⁺: 360.1. Found 360.1.

### Example 133: 5-((3-Cyclopropyl-1H-indazol-5-yl)oxy)-5,6,7,8-tetrahydroquinoline-2-carbonitrile, enantiomer 1 and 2

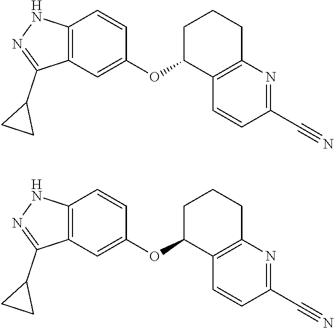 [SMILES: N#Cc1ccc2c(n1)CCC[C@H]2Oc1ccc2[nH]nc(C3CC3)c2c1 ; N#Cc1ccc2c(n1)CCC[C@@H]2Oc1ccc2[nH]nc(C3CC3)c2c1]

### Step 1:5-Hydroxy-5,6,7,8-tetrahydroquinoline-2-carbonitrile

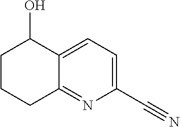 [SMILES: N#Cc1ccc2c(n1)CCCC2O]

5-Oxo-5,6,7,8-tetrahydroquinoline-2-carbonitrile (250.0 mg, 1.45 mmol) was dissolved in MeOH (25 mL), then sodium borohydride (109.9 mg, 2.90 mmol) was added and the mixture was stirred at r.t. for 1 hr. The solvent was evaporated and the residue was partitioned between H₂O and EtOAc. The phases were separated and the aqueous layer was extracted with EtOAc (2×). The combined organic phases were washed with H₂O (1×), dried over Na₂SO₄ and evaporated to dryness to afford the title compound (220 mg, 87%) as a white solid. ¹H NMR (400 MHz, DMSO-d₆) δ 7.95-8.05 (m, 1H) 7.84 (d, J=7.92 Hz, 1H) 5.60 (d, J=5.94 Hz, 1H) 4.61-4.77 (m, 1H) 2.75-2.95 (m, 2H) 1.91-2.06 (m, 2H) 1.73-1.86 (m, 1H) 1.60-1.72 (m, 1H). MS-ESI (m/z) calc'd for C₁₀H₁₁N₂O [M+H]⁺: 175.0. Found 175.0.

### Step 2: tert-Butyl 5-((2-cyano-5,6,7,8-tetrahydroquinolin-5-yl)oxy)-3-iodo-1H-indazole-1-carboxylate

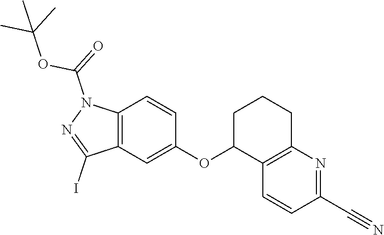 [SMILES: CC(C)(C)OC(=O)n1nc(I)c2cc(OC3CCCc4nc(C#N)ccc43)ccc21]

Diethyl azodicarboxylate (219.9 mg, 1.26 mmol) was added dropwise to a stirred solution of 5-hydroxy-5,6,7,8-tetrahydroquinoline-2-carbonitrile (220.0 mg, 1.26 mmol), tert-butyl 5-hydroxy-3-iodo-1H-indazole-1-carboxylate (454.8 mg, 1.26 mmol) and triphenylphosphine (364.4 mg, 1.39 mmol) in THF (5.5 mL) at 0° C. The reaction mixture was stirred for 15 minutes at 0° C. and then warmed to r.t. and stirred for an additional 2 hrs. The reaction mixture was partitioned between H₂O and EtOAc and the phases were separated. The aqueous layer was extracted with EtOAc (2×) and the combined organic phases were washed with H₂O (1×), dried over anhydrous Na₂SO₄ and evaporated to dryness. The material was purified by chromatography, first on a 25 g silica gel column using a 0-30% EtOAc/cyclohexane gradient eluent and then on a 28 g NH silica gel column using a 0-30% EtOAc/cyclohexane gradient eluent to afford the title compound (460 mg, 70%) as a white solid. ¹H NMR (400 MHz, DMSO-d₆) δ 8.02 (dd, J=9.79, 8.69 Hz, 2H) 7.89 (d, J=7.92 Hz, 1H) 7.44 (dd, J=9.02, 2.42 Hz, 1H) 7.21 (d, J=2.42 Hz, 1H) 5.81 (t, J=4.73 Hz, 1H) 2.87-3.07 (m, 2H) 1.86-2.16 (m, 4H) 1.65 (s, 9H). MS-ESI (m/z) calc'd for C₂₂H₂₂IN₄O₃ [M+H]⁺: 517.1. Found 517.1.

### Step 3: tert-Butyl 5-((2-cyano-5,6,7,8-tetrahydroquinolin-5-yl)oxy)-3-cyclopropyl-1H-indazole-1-carboxylate

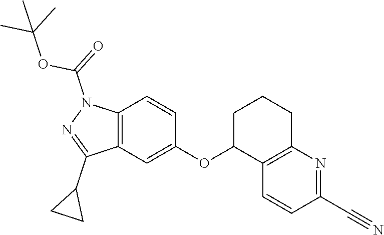 [SMILES: CC(C)(C)OC(=O)n1nc(C2CC2)c2cc(OC3CCCc4nc(C#N)ccc43)ccc21]

tert-Butyl 5-((2-cyano-5,6,7,8-tetrahydroquinolin-5-yl)oxy)-3-iodo-1H-indazole-1-carboxylate (120.0 mg, 0.23 mmol), cyclopropylboronic acid (29.9 mg, 0.35 mmol) and tripotassium phosphate (96.4 mg, 0.70 mmol) were dissolved in 1,4-dioxane (1.7 mL) and the mixture was degassed with N₂ for 5 minutes. [1,1′-bis(Diphenylphosphino)ferrocene]dichloropalladium(II) (17.0 mg, 0.02 mmol) was added and the mixture was stirred at 100° C. under N₂ for 3 hrs. The reaction mixture was partitioned between H₂O and EtOAc and the phases were separated. The aqueous layer was extracted with EtOAc (2×) and the combined organic phases were washed with H₂O (1×), dried over Na₂SO₄ and evaporated to dryness. The residue was purified by chromatography, first on a 10 g silica gel column using a 0-30% EtOAc/cyclohexane gradient eluent, then on a 5 g NH silica gel column 0-40% EtOAc/cyclohexane gradient eluent to afford the title compound (45 mg, 45%) as a white solid. ¹H NMR (400 MHz, DMSO-d₆) δ 8.03 (d, J=7.92 Hz, 1H) 7.91 (dd, J=16.95, 8.58 Hz, 2H) 7.67 (d, J=2.20 Hz, 1H) 7.34 (dd, J=9.24, 2.42 Hz, 1H) 5.73 (t, J=4.95 Hz, 1H) 2.87-3.07 (m, 2H) 2.34-2.44 (m, 1H) 1.97-2.17 (m, 3H) 1.86-1.96 (m, 1H) 1.63 (s, 9H) 0.98-1.14 (m, 4H). MS-ESI (m/z) calc'd for C₂₅H₂₇N₄O₃ [M+H]⁺: 431.2. Found 431.2.

### Step 4: 5-((3-Cyclopropyl-1H-indazol-5-yl)oxy)-5,6,7,8-tetrahydroquinoline-2-carbonitrile, enantiomer 1 and 2

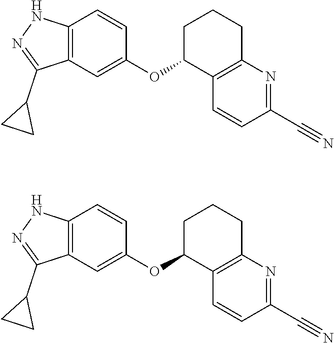 [SMILES: N#Cc1ccc2c(n1)CCC[C@H]2Oc1ccc2[nH]nc(C3CC3)c2c1 ; N#Cc1ccc2c(n1)CCC[C@@H]2Oc1ccc2[nH]nc(C3CC3)c2c1]

To a solution of tert-butyl 5-((2-cyano-5,6,7,8-tetrahydroquinolin-5-yl)oxy)-3-cyclopropyl-1H-indazole-1-carboxylate (45.0 mg, 0.09 mmol) in DCM (1 mL) was added TFA (0.5 mL) and the mixture was stirred at r.t. for 1 hr. The reaction mixture was evaporated to dryness to afford 5-((3-cyclopropyl-1H-indazol-5-yl)oxy)-5,6,7,8-tetrahydroquinoline-2-carbonitrile (38 mg) which was subjected to chiral separation using Method DW to afford 5-((3-cyclopropyl-1H-indazol-5-yl)oxy)-5,6,7,8-tetrahydroquinoline-2-carbonitrile, enantiomer 1 (4.8 mg, 15%) as a light yellow solid. ¹H NMR (400 MHz, DMSO-d₆) δ 12.44 (s, 1H) 8.03 (d, J=7.92 Hz, 1H) 7.88 (d, J=7.92 Hz, 1H) 7.46 (d, J=1.98 Hz, 1H) 7.39 (d, J=8.80 Hz, 1H) 7.09 (dd, J=9.02, 2.20 Hz, 1H) 5.59 (t, J=4.95 Hz, 1H) 2.86-3.08 (m, 2H) 2.19-2.30 (m, 1H) 1.96-2.16 (m, 3H) 1.82-1.96 (m, 1H) 0.85-1.03 (m, 4H). MS-ESI (m/z) calc'd for C₂₀H₁₉N₄O [M+H]⁺: 331.1. Found 331.1. A later eluting fraction was also isolated to afford 5-((3-cyclopropyl-1H-indazol-5-yl)oxy)-5,6,7,8-tetrahydroquinoline-2-carbonitrile, enantiomer 2 (5.5 mg, 18%) as a light yellow solid. ¹H NMR (400 MHz, DMSO-d₆) δ 12.44 (s, 1H) 8.03 (d, J=7.92 Hz, 1H) 7.88 (d, J=7.92 Hz, 1H) 7.46 (d, J=1.76 Hz, 1H) 7.39 (d, J=9.02 Hz, 1H) 7.10 (dd, J=9.02, 2.20 Hz, 1H) 5.59 (t, J=4.84 Hz, 1H) 2.84-3.09 (m, 2H) 2.19-2.31 (m, 1H) 1.96-2.15 (m, 3H) 1.82-1.94 (m, 1H) 0.87-1.02 (m, 4H). MS-ESI (m/z) calc'd for C₂₀H₁₉N₄O [M+H]⁺: 331.1. Found 331.1.

### Example 134: 1-((3-Cyclopropyl-1H-indazol-5-yl)amino)-2,3-dihydro-1H-indene-5-carbonitrile

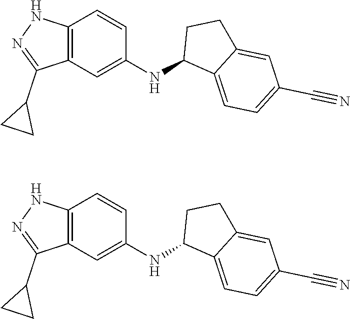 [SMILES: N#Cc1ccc2c(c1)CC[C@@H]2Nc1ccc2[nH]nc(C3CC3)c2c1 ; N#Cc1ccc2c(c1)CC[C@H]2Nc1ccc2[nH]nc(C3CC3)c2c1]

### Step 1: tert-Butyl 5-((5-cyano-2,3-dihydro-1H-inden-1-yl)amino)-3-iodo-1H-indazole-1-carboxylate

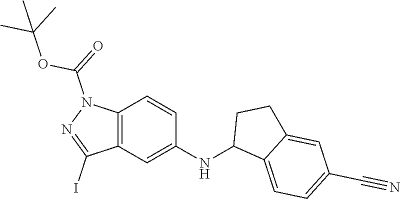 [SMILES: CC(C)(C)OC(=O)n1nc(I)c2cc(NC3CCc4cc(C#N)ccc43)ccc21]

To a solution of 1-((3-iodo-1H-indazol-5-yl)amino)-2,3-dihydro-1H-indene-5-carbonitrile (200.0 mg, 0.50 mmol) and triethylamine (0.08 mL, 0.60 mmol) in THF (3.76 mL), di-tert-butyl dicarbonate (239.9 mg, 1.10 mmol) was added and the mixture was stirred at 25° C. for 3 hrs. The reaction mixture was partitioned between H₂O and EtOAc and the phases were separated. The aqueous layer was extracted with EtOAc (2×) and the combined organic phases washed with brine (1×), dried over anhydrous Na₂SO₄, filtered and evaporated to dryness. The residue was purified by chromatography on a 10 g silica gel column using a 0-30% EtOAc/cyclohexane gradient eluent to afford the title compound (107 mg, 43%). ¹H NMR (400 MHz, DMSO-d₆) δ 7.82 (d, J=9.0 Hz, 1H), 7.76 (s, 1H), 7.65 (d, J=7.8 Hz, 1H), 7.43 (d, J=7.8 Hz, 1H), 7.18 (dd, J=9.1, 2.3 Hz, 1H), 6.62 (d, J=2.2 Hz, 1H), 6.42 (d, J=8.7 Hz, 1H), 5.19 (q, J=8.0 Hz, 1H), 3.01 (ddd, J=12.1, 8.8, 4.4 Hz, 1H), 2.92 (dt, J=16.2, 8.3 Hz, 1H), 2.59 (dtd, J=11.4, 7.6, 3.5 Hz, 1H), 1.86 (dq, J=12.5, 8.6 Hz, 1H), 1.63 (s, 9H). MS-ESI (m/z) calc'd for C₂₂H₂₂IN₄O₂ [M+H]⁺: 501.0. Found 501.0.

### Step 2: tert-Butyl 5-((5-cyano-2,3-dihydro-1H-inden-1-yl)amino)-3-cyclopropyl-1H-indazole-1-carboxylate

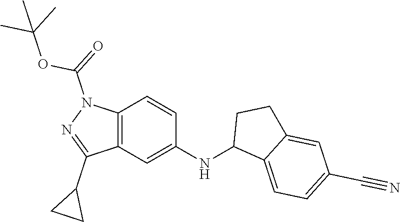 [SMILES: CC(C)(C)OC(=O)n1nc(C2CC2)c2cc(NC3CCc4cc(C#N)ccc43)ccc21]

In a sealed MW vial, tert-butyl 5-((5-cyano-2,3-dihydro-1H-inden-1-yl)amino)-3-iodo-1H-indazole-1-carboxylate (100.0 mg, 0.20 mmol), tripotassium phosphate (127.3 mg, 0.60 mmol) and cyclopropylboronic acid (34.3 mg, 0.40 mmol) were dissolved in 1,4-dioxane (1.46 mL). The mixture was degassed with N₂ for 15 minutes. Then [1,1′-bis(diphenylphosphino)ferrocene]dichloropalladium(II) (14.7 mg, 0.02 mmol) was added and the mixture was stirred at 95° C. for 4 hrs. The reaction mixture was partitioned between H₂O and EtOAc, the phases were separated, the aqueous layer was extracted with EtOAc (2×) and the combined organic phases washed with brine (1×), dried over anhydrous Na₂SO₄, filtered and evaporated to dryness. The residue was purified by chromatography on a 10 g silica gel column, column using a 0-50% EtOAc/cyclohexane gradient eluent to afford the title compound (58 mg, 70%) as a pale yellow oil. ¹H NMR (400 MHz, CDCl₃) δ 7.88 (d, J=9.1 Hz, 1H), 7.57 (s, 1H), 7.51 (d, J=7.9 Hz, 1H), 7.47 (d, J=7.9 Hz, 1H), 6.96-6.89 (m, 2H), 5.22-5.03 (m, 1H), 3.93 (s, 1H), 3.08 (ddd, J=16.5, 8.7, 3.7 Hz, 1H), 3.02-2.91 (m, 1H), 2.77-2.66 (m, 1H), 2.19-2.09 (m, 1H), 2.03-1.91 (m, 1H), 1.70 (s, 9H), 1.22-1.16 (m, 2H), 1.07-0.99 (m, 2H). MS-ESI (m/z) calc'd for C₂₅H₂₇N₄O₂ [M+H]⁺: 415.2. Found 415.2.

### Step 3: 1-((3-cyclopropyl-1H-indazol-5-yl)amino)-2,3-dihydro-1H-indene-5-carbonitrile, enantiomer 1 and 2

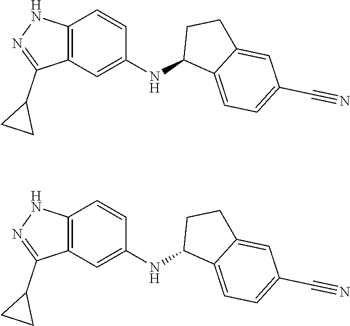 [SMILES: N#Cc1ccc2c(c1)CC[C@@H]2Nc1ccc2[nH]nc(C3CC3)c2c1 ; N#Cc1ccc2c(c1)CC[C@H]2Nc1ccc2[nH]nc(C3CC3)c2c1]

Prepared as described for 5-((3-cyclopropyl-1H-indazol-5-yl)oxy)-5,6,7,8-tetrahydroquinoline-2-carbonitrile, using tert-butyl 5-((5-cyano-2,3-dihydro-1H-inden-1-yl)amino)-3-cyclopropyl-1H-indazole-1-carboxylate in place of tert-butyl 5-[(2-cyano-5,6,7,8-tetrahydroquinolin-5-yl)oxy]-3-cyclopropyl-1H-indazole-1-carboxylate, to afford 1-((3-cyclopropyl-1H-indazol-5-yl)amino)-2,3-dihydro-1H-indene-5-carbonitrile (40 mg, 69%). The racemic mixture was subjected to chiral separation using Method DX to afford 1-((3-cyclopropyl-1H-indazol-5-yl)amino)-2,3-dihydro-1H-indene-5-carbonitrile, enantiomer 1 (12.7 mg, 22%) as a white solid. ¹H NMR (400 MHz, DMSO-d₆) δ 12.11 (s, 1H), 7.74 (s, 1H), 7.67-7.58 (m, 1H), 7.45 (d, J=7.8 Hz, 1H), 7.31-7.13 (m, 1H), 7.00-6.78 (m, 2H), 5.70 (d, J=8.9 Hz, 1H), 5.10 (q, J=7.9 Hz, 1H), 3.01 (ddd, J=16.1, 8.7, 3.3 Hz, 1H), 2.95-2.84 (m, 1H), 2.63-2.53 (m, 1H), 2.21-2.05 (m, 1H), 1.94-1.75 (m, 1H), 0.98-0.77 (m, 4H). MS-ESI (m/z) calc'd for C₂₀H₁₉N₄ [M+H]⁺: 315.1. Found 315.1. A later eluting fraction was also isolated to afford 1-((3-cyclopropyl-1H-indazol-5-yl)amino)-2,3-dihydro-1H-indene-5-carbonitrile, enantiomer 2 (12.8 mg, 22%) as a white solid. ¹H NMR (400 MHz, DMSO-d₆) δ 12.11 (s, 1H), 7.74 (s, 1H), 7.63 (dd, J=7.8, 1.5 Hz, 1H), 7.45 (d, J=7.8 Hz, 1H), 7.25-7.18 (m, 1H), 6.94-6.84 (m, 2H), 5.71 (d, J=8.9 Hz, 1H), 5.10 (q, J=7.9 Hz, 1H), 3.01 (ddd, J=16.2, 8.7, 3.3 Hz, 1H), 2.95-2.83 (m, 1H), 2.65-2.53 (m, 1H), 2.20-2.06 (m, 1H), 1.94-1.76 (m, 1H), 0.99-0.81 (m, 4H). MS-ESI (m/z) calc'd for C₂₀H₁₉N₄ [M+H]⁺: 315.1. Found 315.1.

### Example 135: 5-((3-Iodo-1H-indazol-5-yl)oxy)-5,6,7,8-tetrahydroquinoline-2-carbonitrile

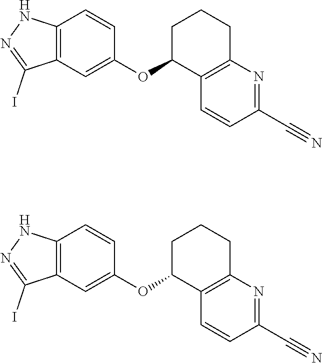 [SMILES: N#Cc1ccc2c(n1)CCC[C@@H]2Oc1ccc2[nH]nc(I)c2c1 ; N#Cc1ccc2c(n1)CCC[C@H]2Oc1ccc2[nH]nc(I)c2c1]

Prepared as described for 5-[(3-cyclopropyl-1H-indazol-5-yl)oxy]-5,6,7,8-tetrahydroquinoline-2-carbonitrile using tert-butyl 5-((2-cyano-5,6,7,8-tetrahydroquinolin-5-yl)oxy)-3-iodo-1H-indazole-1-carboxylate in place of tert-butyl 5-[(2-cyano-5,6,7,8-tetrahydroquinolin-5-yl)oxy]-3-cyclopropyl-1H-indazole-1-carboxylate to afford rac-5-[(3-iodo-1H-indazol-5-yl)oxy]-5,6,7,8-tetrahydroquinoline-2-carbonitrile (80 mg) which was subjected to chiral separation using Method DY to afford 5-((3-iodo-1H-indazol-5-yl)oxy)-5,6,7,8-tetrahydroquinoline-2-carbonitrile, enantiomer 1 (23.7 mg, 37%) as a white solid. ¹H NMR (400 MHz, DMSO-d₆) δ 13.43 (br. s., 1H) 8.03 (d, J=8.14 Hz, 1H) 7.88 (d, J=7.92 Hz, 1H) 7.52 (d, J=9.02 Hz, 1H) 7.20 (dd, J=8.91, 2.31 Hz, 1H) 7.07 (d, J=2.20 Hz, 1H) 5.69 (t, J=4.73 Hz, 1H) 2.85-3.07 (m, 2H) 1.83-2.15 (m, 4H). MS-ESI (m/z) calc'd for C₁₇H₁₄IN₄O [M+H]⁺: 417.0. Found 417.0. A later eluting fraction was also isolated to afford 5-((3-iodo-1H-indazol-5-yl)oxy)-5,6,7,8-tetrahydroquinoline-2-carbonitrile, enantiomer 2 (23.4 mg, 36%) as a white solid. ¹H NMR (400 MHz, DMSO-d₆) δ 13.42 (br. s., 1H) 8.03 (d, J=7.92 Hz, 1H) 7.88 (d, J=7.92 Hz, 1H) 7.52 (d, J=9.02 Hz, 1H) 7.20 (dd, J=8.91, 2.31 Hz, 1H) 7.07 (d, J=2.20 Hz, 1H) 5.69 (t, J=4.84 Hz, 1H) 2.85-3.07 (m, 2H) 1.85-2.15 (m, 4H). MS-ESI (m/z) calc'd for C₁₇H₁₄IN₄O [M+H]⁺: 417.0. Found 417.0.

### Example 136: 8-((3-Cyclopropyl-1H-indazol-5-yl)oxy)-5,6,7,8-tetrahydroquinoline-3-carbonitrile, enantiomer 1 and 2

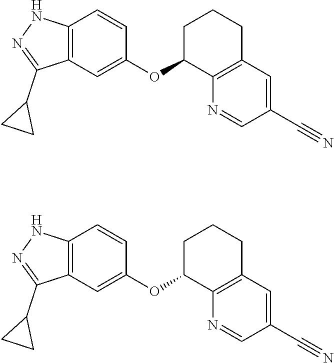 [SMILES: N#Cc1cnc2c(c1)CCC[C@@H]2Oc1ccc2[nH]nc(C3CC3)c2c1 ; N#Cc1cnc2c(c1)CCC[C@H]2Oc1ccc2[nH]nc(C3CC3)c2c1]

### Step 1: tert-Butyl 5-((3-cyano-5,6,7,8-tetrahydroquinolin-8-yl)oxy)-3-cyclopropyl-1H-indazole-1-carboxylate

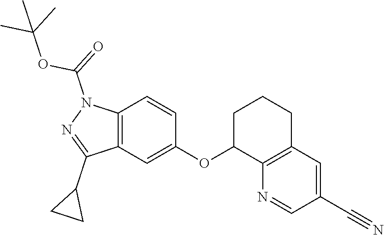 [SMILES: CC(C)(C)OC(=O)n1nc(C2CC2)c2cc(OC3CCCc4cc(C#N)cnc43)ccc21]

tert-Butyl 5-((3-cyano-5,6,7,8-tetrahydroquinolin-8-yl)oxy)-3-iodo-1H-indazole-1-carboxylate (120.0 mg, 0.23 mmol), cyclopropylboronic acid (30.0 mg, 0.35 mmol) and K₃PO₄ (96.36 mg, 0.70 mmol) were dissolved in 1,4-dioxane (2 mL) then the mixture was degassed with N₂ for 5 minutes. Pd(dppf)Cl₂ (17.01 mg, 0.02 mmol) was then added and the mixture was stirred at 100° C. under N₂ for 3 hrs. The reaction mixture was partitioned between H₂O and EtOAc, the phases were separated, and the aqueous layer was extracted with EtOAc (2×). The combined organic phases were washed with H₂O, dried over anhydrous Na₂SO₄ and evaporated to dryness. The residue was purified by chromatography on a 5 g silica gel column using a 0-30% EtOAc/cyclohexane gradient eluent to afford the title compound (74 mg, 74%) as a white solid. ¹H NMR (400 MHz, DMSO-d₆) δ 8.86 (d, J=1.98 Hz, 1H) 8.21 (d, J=1.98 Hz, 1H) 7.90 (d, J=9.02 Hz, 1H) 7.65 (d, J=2.42 Hz, 1H) 7.31 (dd, J=9.02, 2.42 Hz, 1H) 5.62 (t, J=3.96 Hz, 1H) 2.91-3.04 (m, 1H) 2.78-2.90 (m, 1H) 2.31-2.43 (m, 1H) 2.20-2.30 (m, 1H) 2.00-2.11 (m, 1H) 1.89-1.99 (m, 1H) 1.84 (d, J=9.46 Hz, 1H) 1.63 (s, 9H) 0.99-1.15 (m, 4H). MS-ESI (m/z) calc'd for C₂₅H₂₇N₄O₃ [M+H]⁺: 431.2. Found 431.2.

### Step 2: 8-((3-Cyclopropyl-1H-indazol-5-yl)oxy)-5,6,7,8-tetrahydroquinoline-3-carbonitrile, enantiomer 1 and 2

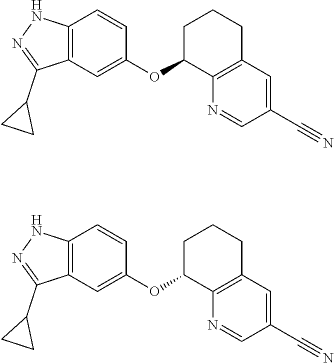 [SMILES: N#Cc1cnc2c(c1)CCC[C@@H]2Oc1ccc2[nH]nc(C3CC3)c2c1 ; N#Cc1cnc2c(c1)CCC[C@H]2Oc1ccc2[nH]nc(C3CC3)c2c1]

Prepared as described for 5-((3-cyclopropyl-1H-indazol-5-yl)oxy)-5,6,7,8-tetrahydroquinoline-2-carbonitrile using tert-butyl 5-((3-cyano-5,6,7,8-tetrahydroquinolin-8-yl)oxy)-3-cyclopropyl-1H-indazole-1-carboxylate in place of tert-butyl 5-((2-cyano-5,6,7,8-tetrahydroquinolin-5-yl)oxy)-3-cyclopropyl-1H-indazole-1-carboxylate, to afford 8-((3-cyclopropyl-1H-indazol-5-yl)oxy)-5,6,7,8-tetrahydroquinoline-3-carbonitrile. The racemic mixture was subjected to chiral separation using Method DZ to afford 8-((3-cyclopropyl-1H-indazol-5-yl)oxy)-5,6,7,8-tetrahydroquinoline-3-carbonitrile, enantiomer 1 (19.0 mg, 3%), as a white solid. ¹H NMR (400 MHz, DMSO-d₆) δ 12.40 (s, 1H) 8.87 (d, J=1.98 Hz, 1H) 8.20 (d, J=1.98 Hz, 1H) 7.45 (d, J=1.98 Hz, 1H) 7.36 (d, J=9.02 Hz, 1H) 7.07 (dd, J=8.91, 2.31 Hz, 1H) 5.47 (t, J=3.41 Hz, 1H) 2.91-3.06 (m, 1H) 2.76-2.89 (m, 1H) 2.16-2.34 (m, 2H) 1.89-2.08 (m, 2H) 1.75-1.87 (m, 1H) 0.85-1.01 (m, 4H). MS-ESI (m/z) calc'd for C₂₀H₂₀N₄O [M+H]⁺: 331.1. Found 331.1. A later eluting fraction was also isolated to afford 8-((3-cyclopropyl-1H-indazol-5-yl)oxy)-5,6,7,8-tetrahydroquinoline-3-carbonitrile, enantiomer 2 (20.0 mg, 35%) as a white solid. ¹H NMR (400 MHz, DMSO-d₆) δ 12.40 (s, 1H) 8.87 (d, J=1.98 Hz, 1H) 8.20 (d, J=1.98 Hz, 1H) 7.45 (d, J=1.98 Hz, 1H) 7.36 (d, J=9.02 Hz, 1H) 7.07 (dd, J=9.02, 2.20 Hz, 1H) 5.47 (t, J=3.19 Hz, 1H) 2.92-3.02 (m, 1H) 2.76-2.91 (m, 1H) 2.20-2.31 (m, 2H) 1.90-2.06 (m, 2H) 1.73-1.88 (m, 1H)_{0.85}-1.01 (m, 4H). MS-ESI (m/z) calc'd for C₂₀H₂₀N₄O [M+H]⁺: 331.1. Found 331.1.

### Example 137: 8-((3-Iodo-1H-indazol-5-yl)oxy)-5,6,7,8-tetrahydroquinoline-3-carbonitrile, enantiomer 1 and 2

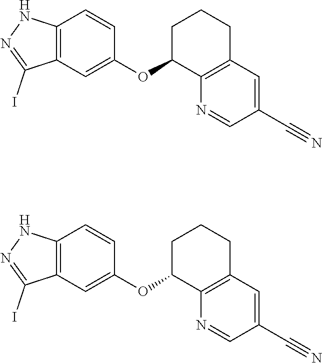 [SMILES: N#Cc1cnc2c(c1)CCC[C@@H]2Oc1ccc2[nH]nc(I)c2c1 ; N#Cc1cnc2c(c1)CCC[C@H]2Oc1ccc2[nH]nc(I)c2c1]

Prepared as described for 5-((3-cyclopropyl-1H-indazol-5-yl)oxy)-5,6,7,8-tetrahydroquinoline-2-carbonitrile, using tert-butyl 5-((5-cyano-2,3-dihydro-1H-inden-1-yl)oxy]-3-iodo-1H-indazole-1-carboxylate in place of tert-butyl 5-((2-cyano-5,6,7,8-tetrahydroquinolin-5-yl)oxy)-3-cyclopropyl-1H-indazole-1-carboxylate, to afford 8-((3-iodo-1H-indazol-5-yl)oxy)-5,6,7,8-tetrahydroquinoline-3-carbonitrile, which was subjected to chiral separation using Method EA to afford 8-((3-iodo-1H-indazol-5-yl)oxy)-5,6,7,8-tetrahydroquinoline-3-carbonitrile, enantiomer 1 (26.0 mg, 41%) as a white solid. ¹H NMR (400 MHz, DMSO-d₆) δ 13.38 (br. s., 1H) 8.86 (d, J=1.98 Hz, 1H) 8.21 (d, J=1.98 Hz, 1H) 7.48 (d, J=9.02 Hz, 1H) 7.17 (dd, J=8.91, 2.31 Hz, 1H) 7.07 (d, J=2.20 Hz, 1H) 5.54 (t, J=3.85 Hz, 1H) 2.92-3.03 (m, 1H) 2.76-2.89 (m, 1H) 2.19-2.31 (m, 1H) 1.77-2.11 (m, 3H). MS-ESI (m/z) calc'd for C₁₇H₁₄IN₄O [M+H]⁺: 417.0. Found 417.0. A later eluting fraction was also isolated to afford 8-((3-iodo-1H-indazol-5-yl)oxy)-5,6,7,8-tetrahydroquinoline-3-carbonitrile, enantiomer 2 (26.0 mg, 41%) as a white solid. ¹H NMR (400 MHz, DMSO-d₆) δ 13.39 (br. s., 1H) 8.86 (d, J=1.98 Hz, 1H) 8.21 (d, J=1.98 Hz, 1H) 7.48 (d, J=9.02 Hz, 1H) 7.17 (dd, J=8.91, 2.31 Hz, 1H) 7.07 (d, J=2.20 Hz, 1H) 5.54 (t, J=3.74 Hz, 1H) 2.91-3.03 (m, 1H) 2.75-2.89 (m, 1H) 2.18-2.29 (m, 1H) 1.78-2.10 (m, 3H). MS-ESI (m/z) calc'd for C₁₇H₁₄IN₄O [M+H]⁺: 417.0. Found 417.0.

### Example 138: 3,3-Difluoro-1-((3-(oxazol-5-yl)-1H-indazol-5-yl)amino)-2,3-dihydro-1H-indene-5-carbonitrile, enantiomer 1 and 2

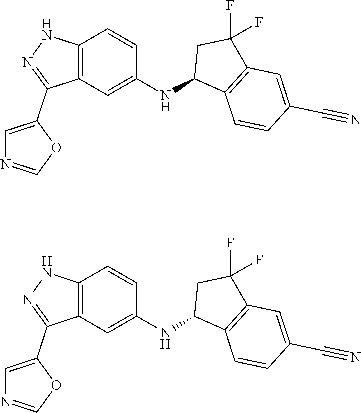 [SMILES: N#Cc1ccc2c(c1)C(F)(F)C[C@@H]2Nc1ccc2[nH]nc(-c3cnco3)c2c1 ; N#Cc1ccc2c(c1)C(F)(F)C[C@H]2Nc1ccc2[nH]nc(-c3cnco3)c2c1]

### Step 1: 2,3-Dihydrospiro[indene-1,2′-[1,3]dithiolane]-6-carbonitrile

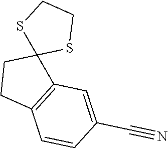 [SMILES: N#Cc1ccc2c(c1)C1(CC2)SCCS1]

To a solution of 3-oxo-2,3-dihydro-1H-indene-5-carbonitrile (2 g, 12.73 mmol) in CH₂Cl₂ (80 mL) were added BF₃.Et₂O (9.03 g, 63.63 mmol), AcOH (6.11 g, 101.80 mmol), and ethane-1,2-dithiol (5.99 g, 63.63 mmol) at 25° C. The mixture was stirred at 25° C. for 1 hr. The mixture was concentrated and purified by flash silica gel column chromatography (ISCO; 40 g SepaFlash column) using a 0-7% EtOAc/petroleum ether gradient eluent to afford the title compound (750 mg, 25%) as a white solid. ¹H NMR (400 MHz, CDCl₃) δ 7.75 (s, 1H), 7.41 (dd, J=7.82, 1.44 Hz, 1H), 7.20 (d, J=8.25 Hz, 1H), 3.34-3.54 (m, 4H), 2.95 (t, J=6.69 Hz, 2H), 2.64 (t, J=6.75 Hz, 2H).

### Step 2: 2-Bromo-3,3-difluoro-2,3-dihydro-1H-indene-5-carbonitrile

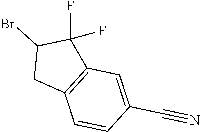 [SMILES: N#Cc1ccc2c(c1)C(F)(F)C(Br)C2]

A solution of 1,3-dibromo-5,5-dimethylimidazolidine-2,4-dione (3.19 g, 11.14 mmol) in CH₂Cl₂ (15 mL) was cooled to −70° C. Pyridine hydrofluoride (3.67 g, 25.91 mmol) was added dropwise at −65° C. under an N₂ atmosphere and the mixture was stirred at −70° C. for 30 min. A solution of 2,3-dihydrospiro[indene-1,2′-[1,3]dithiolane]-6-carbonitrile (650 mg, 2.79 mmol) in CH₂Cl₂ (5 mL) was then added dropwise and the resulting mixture was stirred at −70° C. for 4 hrs, and then stirred at 25° C. for an additional 12 hrs. The reaction mixture was diluted with 1 M HCl to pH=4 and extracted with CH₂Cl₂ (3×). The combined organic layers were dried over Na₂SO₄, filtered, and concentrated under reduced pressure to give a residue. The residue was purified by flash silica gel column chromatography (ISCO; 12 g SepaFlash column) using a 0-7% EtOAc/petroleum ether gradient eluent to afford the title compound (700 mg, 97%) as a yellow solid. ¹H NMR (400 MHz, CDCl₃) δ 7.83 (s, 1H), 7.71 (d, J=7.88 Hz, 1H), 7.37 (br d, J=7.75 Hz, 1H), 4.54 (tt, J=10.51, 7.13 Hz, 1H), 3.60 (ddd, J=17.10, 7.29, 1.63 Hz, 1H), 3.27 (br dd, J=17.01, 6.75 Hz, 1H).

### Step 3: 1,1-Difluoro-1H-indene-6-carbonitrile

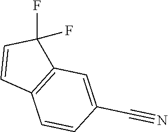 [SMILES: N#Cc1ccc2c(c1)C(F)(F)C=C2]

To a solution of 2-bromo-3,3-difluoro-2,3-dihydro-1H-indene-5-carbonitrile (580 mg, 2.25 mmol) in CH₂Cl₂ (16 mL) was added DBU (547.46 mg, 3.60 mmol) at 25° C. and the mixture was stirred at 25° C. for 2 hrs. The mixture was concentrated and purified by flash silica gel column chromatography (ISCO; 4 g SepaFlash column) using a 0-5% EtOAc/petroleum ether gradient eluent to afford the title compound (267 mg, 67%) as a yellow solid. ¹H NMR (400 MHz, CDCl₃) δ 7.63-7.77 (m, 2H), 7.28 (d, J=7.45 Hz, 1H), 6.84 (br d, J=5.92 Hz, 1H), 6.38 (d, J=5.92 Hz, 1H).

### Step 4: 3,3-Difluoro-2,3-dihydro-1H-indene-5-carbonitrile

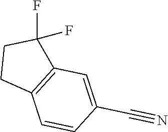 [SMILES: N#Cc1ccc2c(c1)C(F)(F)CC2]

To a solution of 1,1-difluoro-1H-indene-6-carbonitrile (267 mg, 1.51 mmol) in MeCN (5 mL) was added 2-nitrobenzenesulfonyl chloride (668.04 mg, 3.01 mmol) and hydrazine hydrate (307.96 mg, 6.03 mmol) at 0° C. under an N₂ atmosphere. The mixture was stirred at 0° C. for 0.5 hr. Then the mixture was stirred at 25° C. for 12 hrs under an N₂ atmosphere. The mixture was concentrated, diluted with H₂O, and extracted with EtOAc (3×). The combined organic phases were dried over anhydrous Na₂SO₄, filtered, and concentrated to give a residue. The residue was purified by flash silica gel column chromatography (ISCO; 12 g SepaFlash column) using a 0-5% EtOAc/petroleum ether gradient eluent to afford the title compound (270 mg, 100%) as a colorless solid. ¹H NMR (400 MHz, CDCl₃) δ 7.77 (d, J=0.61 Hz, 1H), 7.65 (d, J=8.68 Hz, 1H), 7.36 (dd, J=7.95, 0.61 Hz, 1H), 3.01-3.10 (m, 2H), 2.51-2.63 (m, 2H).

### Step 5: I-Bromo-3,3-difluoro-2,3-dihydro-1H-indene-5-carbonitrile

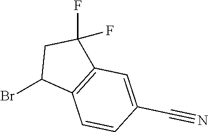 [SMILES: N#Cc1ccc2c(c1)C(F)(F)CC2Br]

A mixture of 3,3-difluoro-2,3-dihydro-1H-indene-5-carbonitrile (120 mg, 669.77 umol), NBS (143.05 mg, 803.72 umol), AIBN (11.00 mg, 66.98 umol) in CCl₄ (5 mL) was degassed and purged with N₂ (3×) at 25° C., and then the mixture was stirred at 80° C. for 12 hrs under an N₂ atmosphere. This procedure was conducted a second time and the residues were combined. The final mixture was concentrated to give a residue. The residue was purified by flash silica gel column chromatography (ISCO; 12 g SepaFlash column) using a 0-5% EtOAc/petroleum ether gradient eluent to afford the title compound (270 mg, 78%) as a colorless oil. ¹H NMR (400 MHz, CDCl₃) δ 7.73-7.77 (m, 2H), 7.58 (br d, J=8.00 Hz, 1H), 5.31-5.40 (m, 1H), 3.15-3.34 (m, 1H), 2.93 (dddd, J=15.62, 13.62, 11.29, 4.25 Hz, 1H).

### Step 6: 3,3-Difluoro-1-((3-(oxazol-5-yl)-1H-indazol-5-yl)amino)-2,3-dihydro-1H-indene-5-carbonitrile

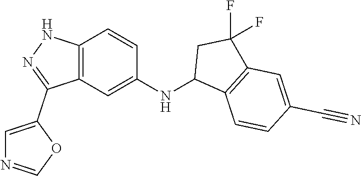 [SMILES: N#Cc1ccc2c(c1)C(F)(F)CC2Nc1ccc2[nH]nc(-c3cnco3)c2c1]

To a solution of 1-bromo-3,3-difluoro-2,3-dihydro-1H-indene-5-carbonitrile (120 mg, 465.00 umol) in DMF (2 mL) was added DIEA (90.15 mg, 697.51 umol) and 3-(oxazol-5-yl)-1H-indazol-5-amine (93.09 mg, 465.00 umol) at 25° C. The mixture was stirred at 25° C. for 3 hrs. The reaction was filtered and the filtrate was concentrated. The material was purified by preparative-HPLC using Method ED to afford the title compound (30 mg, 17%) as a brown solid. MS-ESI (m/z) calc'd for C₂₀H₁₄F₂N₅O [M+H]⁺: 378.1. Found 378.1.

### Step 7: 3,3-Difluoro-1-((3-(oxazol-5-yl)-1H-indazol-5-yl)amino)-2,3-dihydro-1H-indene-5-carbonitrile, enantiomer 1 and 2

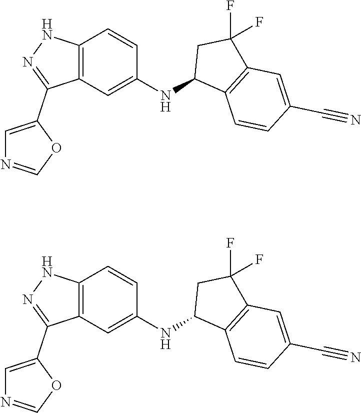 [SMILES: N#Cc1ccc2c(c1)C(F)(F)C[C@@H]2Nc1ccc2[nH]nc(-c3cnco3)c2c1 ; N#Cc1ccc2c(c1)C(F)(F)C[C@H]2Nc1ccc2[nH]nc(-c3cnco3)c2c1]

3,3-Difluoro-1-((3-(oxazol-5-yl)-1H-indazol-5-yl)amino)-2,3-dihydro-1H-indene-5-carbonitrile (9 mg) was separated by SFC method using Method EE to afford 3,3-difluoro-1-((3-(oxazol-5-yl)-1H-indazol-5-yl)amino)-2,3-dihydro-1H-indene-5-carbonitrile, enantiomer 1 (3.96 mg, 44%) as a pale pink solid. ¹H NMR (400 MHz, DMSO-d₆) δ 13.17 (s, 1H), 8.46 (s, 1H), 8.27 (s, 1H), 8.03 (d, J=7.95 Hz, 1H), 7.76 (s, 1H), 7.68 (d, J=7.95 Hz, 1H), 7.43 (d, J=8.93 Hz, 1H), 7.17 (s, 1H), 7.01 (dd, J=9.05, 1.96 Hz, 1H), 6.28 (d, J=9.17 Hz, 1H), 5.52 (br s, 1H), 3.26 (br d, J=7.46 Hz, 1H), 2.39-2.46 (m, 1H). MS-ESI (m/z) calc'd for C₂₀H₁₄F₂N₅O [M+H]⁺: 378.1. Found 378.1. A later eluting fraction was also isolated to afford 3,3-difluoro-1-((3-(oxazol-5-yl)-1H-indazol-5-yl)amino)-2,3-dihydro-1H-indene-5-carbonitrile, enantiomer 2 (3.74 mg, 41%) as a pale pink solid. ¹H NMR (400 MHz, DMSO-d₆) δ 13.18 (s, 1H), 8.46 (s, 1H), 8.26 (s, 1H), 8.03 (d, J=8.07 Hz, 1H), 7.76 (s, 1H), 7.68 (d, J=8.07 Hz, 1H), 7.43 (d, J=8.93 Hz, 1H), 7.17 (s, 1H), 7.01 (dd, J=8.99, 2.02 Hz, 1H), 6.28 (d, J=9.17 Hz, 1H), 5.52 (br s, 1H), 3.26 (br d, J=6.85 Hz, 1H), 2.39-2.46 (m, 1H). MS-ESI (m/z) calc'd for C₂₀H₁₄F₂N₅O [M+H]⁺: 378.1. Found 378.1.

### Example 139: 5-((3-(Cyclopropylmethyl)-1H-indazol-5-yl)amino)-5,6,7,8-tetrahydronaphthalene-2-carbonitrile, enantiomer 1 and 2

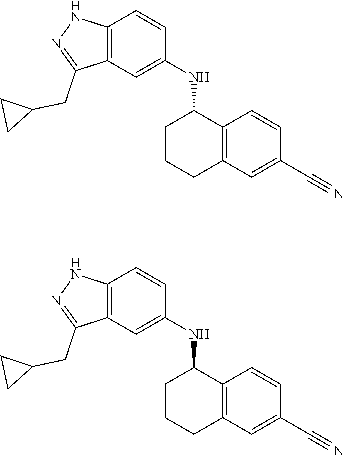 [SMILES: N#Cc1ccc2c(c1)CCC[C@@H]2Nc1ccc2[nH]nc(CC3CC3)c2c1 ; N#Cc1ccc2c(c1)CCC[C@H]2Nc1ccc2[nH]nc(CC3CC3)c2c1]

### Step 1: 2-Cyclopropyl-N-methoxy-N-methylacetamide

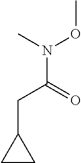

To a solution of 2-cyclopropylacetic acid (5 g, 49.94 mmol) in DCM (60 mL) was added CDI (8.91 g, 54.94 mmol). The mixture was stirred at 20° C. for 1 hr under an N₂ atmosphere. Then N,O-dimethylhydroxylamine (3.36 g, 54.94 mmol) was added. The mixture was stirred at 20° C. for 12 hrs under an N₂ atmosphere. The reaction mixture was concentrated and purified by flash silica gel chromatography (ISCO; 20 g SepaFlash column) using a 0-5% EtOAc/petroleum ether gradient eluent to afford the title compound (4.1 g, 57%) as a pale yellow oil. ¹H NMR (400 MHz, MeOD) δ 4.00 (br s, 3H), 3.47 (br s, 3H), 2.64 (br d, J=7.28 Hz, 2H), 1.26-1.39 (m, 1H), 0.75-0.84 (m, 2H), 0.39-0.50 (m, 2H). MS-ESI (m/z) calc'd for C₇H₁₄NO₂. [M+H]⁺: 144.1. Found 144.1.

### Step 2: 1-(5-Bromo-2-fluorophenyl)-2-cyclopropylethanone

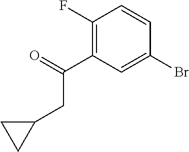 [SMILES: O=C(CC1CC1)c1cc(Br)ccc1F]

To a solution of 4-bromo-1-fluoro-2-iodobenzene (10.09 g, 33.52 mmol) in THF (150 mL) was added i-PrMgCl (2 M, 22.07 mL) at 0° C. under an N₂ atmosphere. The mixture was stirred at 0° C. for 0.5 hr under an N₂ atmosphere. Then 2-cyclopropyl-N-methoxy-N-methylacetamide (4 g, 27.94 mmol) in THF (90 mL) was added at 0° C. under an N₂ atmosphere. The mixture was stirred at 20° C. for 12 hrs under an N₂ atmosphere. The reaction mixture was quenched with saturated NH₄Cl and extracted with EtOAc (3×). The combined organic layer was dried over Na₂SO₄, filtered and concentrated to give a residue. The residue was purified by flash silica gel chromatography (ISCO; 40 g SepaFlash column) using a 0-11% EtOAc/petroleum ether gradient eluent to afford the title compound (5 g, 70%) as a pale yellow oil. ¹H NMR (400 MHz, MeOD) δ 7.92 (dd, J=6.38, 2.63 Hz, 1H), 7.72 (ddd, J=8.76, 4.32, 2.69 Hz, 1H), 7.19 (dd, J=10.63, 8.88 Hz, 1H), 2.88 (dd, J=6.82, 2.81 Hz, 2H), 1.04-1.13 (m, 1H), 0.52-0.59 (m, 2H), 0.14-0.19 (m, 2H). MS-ESI (m/z) calc'd for C₁₁H₁₁BrFO [M+H]⁺: 257.0/259.0. Found 257.0/259.0.

### Step 3: 5-Bromo-3-(cyclopropylmethyl)-1H-indazole

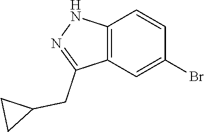 [SMILES: Brc1ccc2[nH]nc(CC3CC3)c2c1]

A mixture of 1-(5-bromo-2-fluorophenyl)-2-cyclopropylethanone (3.8 g, 14.78 mmol) in NH₂NH₂.H₂O (81.39 g, 1.59 mol) was stirred at 100° C. for 24 hrs. The reaction mixture was then diluted with H₂O and extracted with EtOAc (5×). The combined organic layers were dried over Na₂SO₄, filtered and concentrated to afford the title compound (2.7 g, 73%) as a yellow solid. ¹H NMR (400 MHz, MeOD) δ 7.92-7.96 (m, 1H), 7.43 (qd, J=8.93, 1.21 Hz, 2H), 2.86 (d, J=6.84 Hz, 2H), 1.08-1.19 (m, 1H), 0.50-0.57 (m, 2H), 0.24-0.31 (m, 2H). MS-ESI (m/z) calc'd for C₁₁H₁₂BrN₂ [M+H]⁺: 251.0/253.0. Found 251.0/253.0.

### Step 4: 5-Bromo-3-(cyclopropylmethyl)-1-(tetrahydro-2H-pyran-2-yl)-1H-indazole

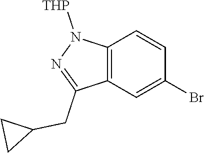

To a solution of 5-bromo-3-(cyclopropylmethyl)-1H-indazole (1.7 g, 6.77 mmol) in DCM (15 mL) were added PTSA (116.57 mg, 676.96 umol) and 3,4-dihydro-2H-pyran (1.14 g, 13.54 mmol) at 20° C. The mixture was stirred at 45° C. for 12 hrs. The reaction mixture was concentrated and purified by flash silica gel chromatography (ISCO; 20 g SepaFlash column) using a 0-5% EtOAc/petroleum ether gradient eluent to afford the title compound (2.1 g, 92%) as a yellow oil. ¹H NMR (400 MHz, MeOD) δ 7.93 (d, J=1.10 Hz, 1H), 7.54-7.58 (m, 1H), 7.45-7.50 (m, 1H), 5.71 (dd, J=9.92, 2.65 Hz, 1H), 3.96-4.03 (m, 1H), 3.78 (td, J=11.14, 2.87 Hz, 1H), 2.85 (d, J=6.62 Hz, 2H), 2.07-2.16 (m, 1H), 1.94-2.01 (m, 1H), 1.60-1.89 (m, 4H), 1.08-1.17 (m, 1H), 0.50-0.56 (m, 2H), 0.25-0.30 (m, 2H). MS-ESI (m/z) calc'd for C₁₆H₂₀BrN₂O. [M+H]⁺: 335.1/337.1. Found 335.0/337.0.

### Step 5: N-(3-(Cyclopropylmethyl)-1-(tetrahydro-2H-pyran-2-yl)-1H-indazol-5-yl)-1,1-diphenylmethanimine

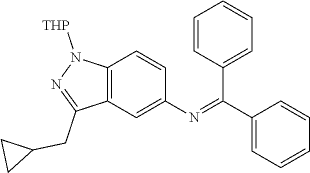

A mixture of 5-bromo-3-(cyclopropylmethyl)-1-(tetrahydro-2H-pyran-2-yl)-1H-indazole (2.1 g, 6.26 mmol), Pd₂(dba)₃ (573.63 mg, 626.42 umol), t-Bu Xphos (CAS: 564483-19-8) (266.00 mg, 626.42 umol), t-BuONa (1.20 g, 12.53 mmol) and diphenylmethanimine (1.14 g, 6.26 mmol) in toluene (40 mL) was degassed and purged with N₂ (3×) at 20° C. The mixture was then stirred at 100° C. for 12 hrs under an N₂ atmosphere. The reaction mixture was concentrated and purified by flash silica gel chromatography (ISCO; 20 g SepaFlash column) using a 0-5% EtOAc/petroleum ether gradient eluent to afford the title compound (2.4 g, 87%) as a yellow oil. ¹H NMR (400 MHz, MeOD) δ 7.67-7.72 (m, 2H), 7.48-7.54 (m, 1H), 7.40-7.46 (m, 3H), 7.20-7.30 (m, 3H), 7.10-7.16 (m, 2H), 6.95-7.02 (m, 2H), 5.64 (dd, J=2.25, 10.26 Hz, 1H), 3.94-4.06 (m, 1H), 3.76 (dt, J=2.69, 11.29 Hz, 1H), 2.71 (d, J=6.75 Hz, 2H), 2.38-2.50 (m, 1H), 2.03-2.14 (m, 1H), 1.94 (br dd, J=2.50, 13.38 Hz, 1H), 1.57-1.86 (m, 3H), 0.90-0.96 (m, 1H), 0.37-0.43 (m, 2H), 0.12 (q, J=4.92 Hz, 2H). MS-ESI (m/z) calc'd for C₂₉H₃₀N₃O. [M+H]⁺: 436.2. Found 436.1.

### Step 6: 3-(Cyclopropylmethyl)-1-(tetrahydro-2H-pyran-2-yl)-1H-indazol-5-amine

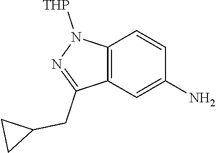

To a solution of N-(3-(cyclopropylmethyl)-1-(tetrahydro-2H-pyran-2-yl)-1H-indazol-5-yl)-1,1-diphenylmethanimine (2.3 g, 5.28 mmol) in THF (40 mL) was added HCl (1 M, 15.84 mL). The mixture was stirred at 20° C. for 10 minutes and then diluted with H₂O and extracted with EtOAc (3×). The combined organic layers were dried over Na₂SO₄, filtered, and the filtrate was concentrated and purified by flash silica gel chromatography (ISCO; 20 g SepaFlash column) using a 0-25% EtOAc/petroleum ether gradient eluent to afford the title compound (1.12 g, 78%) as an orange oil. MS-ESI (m/z) calc'd for C₁₆H₂₂N₃O. [M+H]⁺: 272.2. Found 272.2.

### Step 7: 5-((3-(Cyclopropylmethyl)-1H-indazol-5-yl)amino)-5,6,7,8-tetrahydronaphthalene-2-carbonitrile

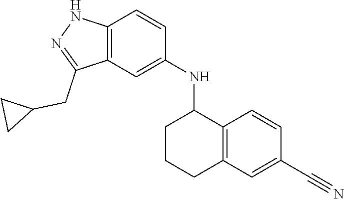 [SMILES: N#Cc1ccc2c(c1)CCCC2Nc1ccc2[nH]nc(CC3CC3)c2c1]

To a solution of 3-(cyclopropylmethyl)-1-(tetrahydro-2H-pyran-2-yl)-1H-indazol-5-amine (200 mg, 737.04 umol) and 5-oxo-5,6,7,8-tetrahydronaphthalene-2-carbonitrile (126.18 mg, 737.04 umol) in DMF (3 mL) were added TMSCl (200.18 mg, 1.84 mmol), BH₃.THF (1 M, 737.04 uL) at 0° C. The mixture was stirred at 0° C. for 2 hrs; then it was warmed to 20° C. and stirred for another 12 hrs. The reaction mixture was diluted with H₂O and extracted with EtOAc (5×). The combined organic layers were dried over Na₂SO₄, filtered, and concentrated to give a residue. The residue was purified by preparative-TLC (SiO₂, petroleum ether/EtOAc=2/1, Rf=0.2) and further purified by preparative-HPLC using Method EF to afford the title compound (9 mg, 3%) as a pale yellow solid. MS-ESI (m/z) calc'd for C₂₂H₂₃N₄. [M+H]⁺: 343.2. Found 343.1.

### Step 8: 5-((3-(Cyclopropylmethyl)-1H-indazol-5-yl)amino)-5,6,7,8-tetrahydronaphthalene-2-carbonitrile, enantiomer 1 and 2

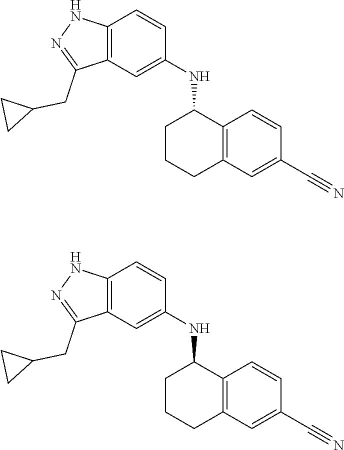 [SMILES: N#Cc1ccc2c(c1)CCC[C@@H]2Nc1ccc2[nH]nc(CC3CC3)c2c1 ; N#Cc1ccc2c(c1)CCC[C@H]2Nc1ccc2[nH]nc(CC3CC3)c2c1]

5-((3-(Cy clopropylmethyl)-1H-indazol-5-yl)amino)-5,6,7,8-tetrahydronaphthalene-2-carbonitrile was subjected to chiral separation using Method EG to afford 5-((3-(cyclopropylmethyl)-1H-indazol-5-yl)amino)-5,6,7,8-tetrahydronaphthalene-2-carbonitrile, enantiomer 1 (3 mg, 33%) as a pale yellow solid. ¹H NMR (400 MHz, DMSO-d₆) δ 12.19 (br s, 1H), 7.62 (s, 1H), 7.49-7.59 (m, 2H), 7.22 (d, J=8.82 Hz, 1H), 6.89 (dd, J=1.98, 8.82 Hz, 1H), 6.80 (s, 1H), 5.65 (d, J=9.04 Hz, 1H), 4.65 (br d, J=6.84 Hz, 1H), 2.77-2.85 (m, 2H), 2.71 (d, J=6.39 Hz, 2H), 1.76-1.99 (m, 4H), 1.00-1.08 (m, 1H), 0.39-0.44 (m, 2H), 0.16-0.21 (m, 2H). MS-ESI (m/z) calc'd for C₂₂H₂₃N₄ [M+H]⁺: 343.2. Found 343.0. A later eluting fraction was also isolated to afford 5-((3-(cyclopropylmethyl)-1H-indazol-5-yl)amino)-5,6,7,8-tetrahydronaphthalene-2-carbonitrile, enantiomer 2 (3.13 mg, 34%) as a pale yellow solid. ¹H NMR (400 MHz, DMSO-d₆) δ 12.19 (br s, 1H), 7.62 (s, 1H), 7.50-7.58 (m, 2H), 7.22 (d, J=8.82 Hz, 1H), 6.89 (dd, J=1.76, 8.82 Hz, 1H), 6.80 (s, 1H), 5.65 (d, J=9.04 Hz, 1H), 4.65 (br d, J=6.84 Hz, 1H), 2.77-2.85 (m, 2H), 2.71 (d, J=6.62 Hz, 2H), 1.73-2.01 (m, 4H), 0.99-1.09 (m, 1H), 0.38-0.45 (m, 2H), 0.15-0.21 (m, 2H). MS-ESI (m/z) calc'd for C₂₂H₂₃N₄. [M+H]⁺: 343.2. Found 343.1.

### Example 140: 3′-((3-(Oxazol-5-yl)-1H-indazol-5-yl)amino)-2′,3′-dihydrospiro[cyclopropane-1,1′-indene]-6′-carbonitrile, enantiomer 1 and 2

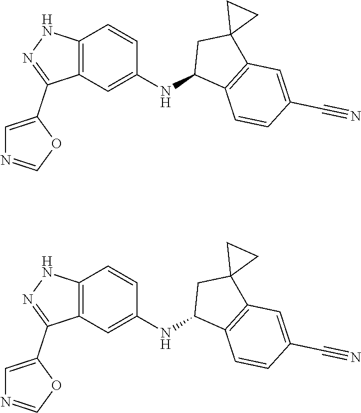 [SMILES: N#Cc1ccc2c(c1)C1(CC1)C[C@@H]2Nc1ccc2[nH]nc(-c3cnco3)c2c1 ; N#Cc1ccc2c(c1)C1(CC1)C[C@H]2Nc1ccc2[nH]nc(-c3cnco3)c2c1]

### Step 1: 6-Bromo-1-methylene-2,3-dihydro-1H-indene

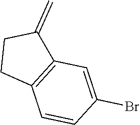

To a solution of methyl(triphenyl)phosphonium bromide (15.23 g, 42.64 mmol) in THF (80 mL) was added t-BuOK (5.02 g, 44.78 mmol) at 20° C. and the mixture was stirred at 20° C. for 0.5 hr. Then 6-bromo-1-methylene-2,3-dihydro-1H-indene (3 g, 14.21 mmol) in THF (20 mL) was added. The resulted mixture was stirred at 20° C. for 12 hrs. The reaction mixture was concentrated to give a residue that was diluted with H₂O and extracted with EtOAc (3×). The combined organic layers were dried over Na₂SO₄, filtered, and concentrated to afford the title compound (2.3 g, 77%) as a pale yellow oil. ¹H NMR (400 MHz, MeOD) δ 7.61 (s, 1H), 7.31 (dd, J=1.65, 8.05 Hz, 1H), 7.15 (d, J=8.16 Hz, 1H), 5.47 (t, J=2.43 Hz, 1H), 5.03-5.08 (m, 1H), 2.86-2.93 (m, 2H), 2.79 (td, J=1.93, 6.28 Hz, 2H).

### Step 2: 6′-Bromo-2′,3′-dihydrospiro[cyclopropane-1,1′-indene]

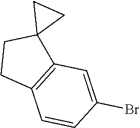 [SMILES: Brc1ccc2c(c1)C1(CC2)CC1]

To a solution of ZnEt₂ (1 M, 38.26 mL) in DCM (30 mL) was added TFA (4.36 g, 38.26 mmol) at 0° C. under an N₂ atmosphere and the mixture was stirred at 0° C. for 15 minutes. Then CH₂I₂ (10.25 g, 38.26 mmol) was added at 0° C. and the mixture was stirred at 0° C. for another 15 minutes under an N₂ atmosphere. Then a solution of 6-bromo-1-methylene-2,3-dihydro-1H-indene (2 g, 9.57 mmol) in DCM (25 mL) was added to the mixture at 0° C. The resulting mixture was stirred at 0° C. for 15 min and then stirred at 20° C. for 12 hrs under an N₂ atmosphere. The reaction mixture was quenched by H₂O and extracted with EtOAc (3×). The combined organic layers were dried over Na₂SO₄, filtered, and concentrated to give a residue. The residue was purified by flash silica gel chromatography (ISCO; 12 g SepaFlash column) using a 0-1% EtOAc/petroleum ether gradient eluent to afford the title compound (2 g, 94%) as a yellow oil. ¹H NMR (400 MHz, MeOD) δ 7.18 (dd, J=8.05, 1.87 Hz, 1H), 7.06 (d, J=7.94 Hz, 1H), 6.79 (d, J=1.76 Hz, 1H), 2.96 (t, J=7.61 Hz, 2H), 2.12 (t, J=7.61 Hz, 2H), 0.97 (s, 2H), 0.86-0.90 (m, 2H).

### Step 3: 6′-Bromospiro[cyclopropane-1,1′-inden]-3′(2′H)-one

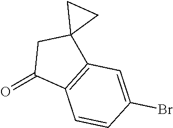 [SMILES: O=C1CC2(CC2)c2cc(Br)ccc21]

To a solution of 6′-bromo-2′,3′-dihydrospiro[cyclopropane-1,1′-indene] (2 g, 8.96 mmol) in acetone (30 mL) was added an aqueous solution of MgSO₄ (1.5 M, 10.16 mL) and KMnO₄ (1.56 g, 9.86 mmol). The mixture was stirred at 20° C. for 12 hrs and then quenched by addition of a 10% aqueous solution of Na₂SO₃ at 0° C. The mixture was filtered and the filtrate was extracted with EtOAc (3×). The combined organic layers were dried over Na₂SO₄, filtered and concentrated to give a residue. The residue was purified by flash silica gel chromatography (ISCO; 20 g SepaFlash column) using a 0-12% EtOAc/petroleum ether gradient eluent to afford the title compound (530 mg, 25%) as a white solid. MS-ESI (m/z) calc'd for C₁₁H₁₀BrO [M+H]⁺: 237.0/239.0. Found 236.9/238.9.

### Step 4: N-(6′-Bromo-2′,3′-dihydrospiro[cyclopropane-1,1′-inden]-3′-yl)-3-(oxazol-5-yl)-1H-indazol-5-amine

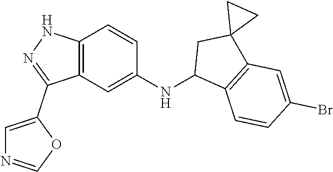 [SMILES: Brc1ccc2c(c1)C1(CC1)CC2Nc1ccc2[nH]nc(-c3cnco3)c2c1]

To a solution of 6′-bromospiro[cyclopropane-1,1′-inden]-3′(2′H)-one (200 mg, 843.55 umol) and 3-(oxazol-5-yl)-1H-indazol-5-amine (168.88 mg, 843.55 umol) in MeOH (15 mL) was added AcOH (101.31 mg, 1.69 mmol) to adjust to pH=5 and the mixture was stirred at 50° C. for 2 hrs. Then NaBH₃CN (159.03 mg, 2.53 mmol) was added and the mixture was stirred at 20° C. for 12 hrs. The reaction mixture was concentrated to give a residue that was purified by flash silica gel chromatography (ISCO; 12 g SepaFlash column) using a 0-21% EtOAc/petroleum ether gradient eluent to afford the title compound (200 mg, 56%) as a pale yellow oil. MS-ESI (m/z) calc'd for C₂₁H₁₈BrN₄O [M+H]⁺: 421.1/423.1. Found 421.0/423.0.

### Step 5: 3′-((3-(Oxazol-5-yl)-1H-indazol-5-yl)amino)-2′,3′-dihydrospiro[cyclopropane-1,1′-indene]-6′-carbonitrile

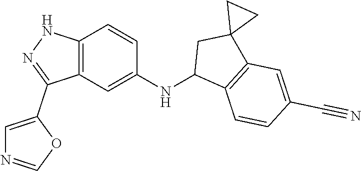 [SMILES: N#Cc1ccc2c(c1)C1(CC1)CC2Nc1ccc2[nH]nc(-c3cnco3)c2c1]

To a solution of N-(6′-bromo-2′,3′-dihydrospiro[cyclopropane-1,1′-inden]-3′-yl)-3-(oxazol-5-yl)-1H-indazol-5-amine (170 mg, 403.52 umol) in DMA (0.5 mL) were added Zn(CN)₂ (94.77 mg, 807.05 umol), Zn (52.77 mg, 807.05 umol), DPPF (44.74 mg, 80.70 umol), and Pd₂(dba)₃ (73.90 mg, 80.70 umol) at 20° C. The mixture was stirred at 100° C. for 2 hrs under an N₂ atmosphere. The reaction mixture was concentrated and then purified by preparative-HPLC using Method EH to afford the title compound (57.74 mg, 30%) as a yellow solid, TFA salt. MS-ESI (m/z) calc'd for C₂₂H₁₈N₅O [M+H]⁺: 368.1. Found 368.0.

### Step 6: 3′-((3-(Oxazol-5-yl)-1H-indazol-5-yl)amino)-2′,3′-dihydrospiro[cyclopropane-1,1′-indene]-6′-carbonitrile, enantiomer 1 and 2

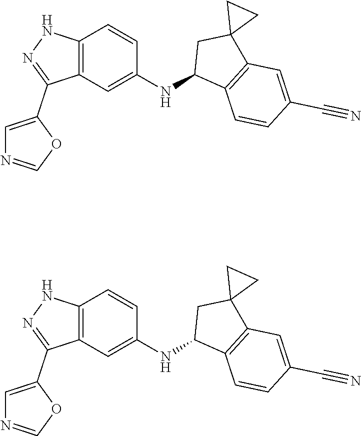 [SMILES: N#Cc1ccc2c(c1)C1(CC1)C[C@@H]2Nc1ccc2[nH]nc(-c3cnco3)c2c1 ; N#Cc1ccc2c(c1)C1(CC1)C[C@H]2Nc1ccc2[nH]nc(-c3cnco3)c2c1]

3′-((3-(Oxazol-5-yl)-1H-indazol-5-yl)amino)-2′,3′-dihydrospiro[cyclopropane-1,1′-indene]-6′-carbonitrile (9 mg) was subjected to chiral separation using Method EI to afford 3′-((3-(oxazol-5-yl)-1H-indazol-5-yl)amino)-2′,3′-dihydrospiro[cyclopropane-1,1′-indene]-6′-carbonitrile, enantiomer 1 (2.37 mg, 20%) as a yellow solid. ¹H NMR (400 MHz, DMSO-d₆) δ 13.12 (br s, 1H), 8.46 (s, 1H), 7.67 (s, 1H), 7.58 (dd, J=1.32, 7.72 Hz, 1H), 7.44 (d, J=7.72 Hz, 1H), 7.34-7.41 (m, 2H), 7.00-7.10 (m, 2H), 6.10 (d, J=8.38 Hz, 1H), 5.38 (q, J=7.86 Hz, 1H), 2.53-2.57 (m, 1H), 2.12 (dd, J=7.28, 12.79 Hz, 1H), 1.14-1.26 (m, 2H), 0.85-0.99 (m, 2H) MS-ESI (m/z) calc'd for C₂₂H₁₈N₅O [M+H]⁺: 368.1. Found 368.0. A later eluting fraction was also isolated to afford 3′-((3-(oxazol-5-yl)-1H-indazol-5-yl)amino)-2′,3′-dihydrospiro[cyclopropane-1,1′-indene]-6′-carbonitrile, enantiomer 2 (2.14 mg, 19%) as a yellow solid. ¹H NMR (400 MHz, DMSO-d₆) δ 13.11 (br s, 1H), 8.45 (s, 1H), 7.66 (s, 1H), 7.58 (dd, J=1.32, 7.94 Hz, 1H), 7.44 (d, J=7.72 Hz, 1H), 7.31-7.41 (m, 2H), 7.08 (s, 1H), 7.02 (dd, J=1.98, 8.82 Hz, 1H), 6.10 (d, J=8.82 Hz, 1H), 5.38 (q, J=7.86 Hz, 1H), 2.53-2.59 (m, 1H), 2.05-2.19 (m, 1H), 1.15-1.27 (m, 2H), 0.85-0.95 (m, 2H). MS-ESI (m/z) calc'd for C₂₂H₁₈N₅O [M+H]⁺: 368.1. Found 368.0.

### Example 141: 8-((3-(1-(Difluoromethyl)-1H-pyrazol-4-yl)-1H-indazol-5-yl)oxy)-5,6,7,8-tetrahydroquinoline-3-carbonitrile, enantiomer 1 and 2

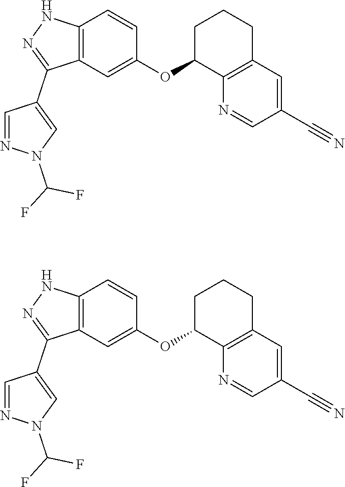 [SMILES: N#Cc1cnc2c(c1)CCC[C@@H]2Oc1ccc2[nH]nc(-c3cnn(C(F)F)c3)c2c1 ; N#Cc1cnc2c(c1)CCC[C@H]2Oc1ccc2[nH]nc(-c3cnn(C(F)F)c3)c2c1]

### Step 1: 3-Bromo-5,6,7,8-tetrahydroquinolin-8-ol

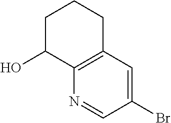 [SMILES: OC1CCCc2cc(Br)cnc21]

To a solution of 3-bromo-6,7-dihydroquinolin-8(5H)-one (190 mg, 840.45 umol) in MeOH (4 mL) was added NaBH₄ (38.16 mg, 1.01 mmol). The mixture was stirred at 20° C. for 1 hr. The reaction mixture was concentrated and purified by preparative-TLC (petroleum ether/EtOAc=1/1, Rf=0.53) to afford the title compound (100 mg, 52%) as a pale yellow oil. MS-ESI (m/z) calc'd for C₉H₁₁BrNO [M+H]⁺:228.0/230.0. Found 228.0/230.0.

### Step 2: 3-Bromo-8-((3-(1-(difluoromethyl)-1H-pyrazol-4-yl)-1H-indazol-5-yl)oxy)-5,6,7,8-tetrahydroquinoline

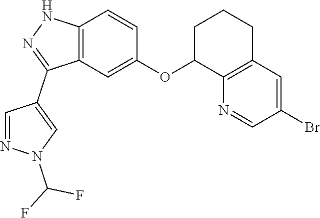 [SMILES: FC(F)n1cc(-c2n[nH]c3ccc(OC4CCCc5cc(Br)cnc54)cc23)cn1]

A mixture of 3-(1-(difluoromethyl)-1H-pyrazol-4-yl)-1H-indazol-5-ol (60.33 mg, 241.14 umol), 3-bromo-5,6,7,8-tetrahydroquinolin-8-ol (50 mg, 219.22 umol), and (trimethylphosphoranylidene)acetonitrile solution (0.5 M in THF, 876.86 uL) in toluene (5 mL) was degassed and purged with N₂ (3×) at 20° C., and then the mixture was stirred at 100° C. for 12 hrs under an N₂ atmosphere. The reaction mixture was concentrated to give a residue. The residue was purified by preparative-TLC (petroleum ether/EtOAc=1/1, Rf=0.46) to afford the title compound (40 mg, 40%) as a yellow oil. ¹H NMR (400 MHz, DMSO-d₆) δ 13.04 (s, 1H), 8.90 (s, 1H), 8.55 (d, 1H, J=2.3 Hz), 8.35 (s, 1H), 7.7-8.0 (m, 2H), 7.67 (d, 1H, J=1.9 Hz), 7.48 (d, 1H, J=9.0 Hz), 7.14 (dd, 1H, J=2.1, 9.0 Hz), 5.61 (br s, 1H), 2.9-3.0 (m, 1H), 2.8-2.8 (m, 1H), 2.2-2.3 (m, 1H), 1.9-2.0 (m, 2H), 1.7-1.8 (m, 1H). MS-ESI (m/z) calc'd for C₂₀H₁₇BrF₂N₅O [M+H]⁺:460.1/462.1. Found 460.0/462.0.

### Step 3: 8-((3-(1-(Difluoromethyl)-1H-pyrazol-4-yl)-1H-indazol-5-yl)oxy)-5,6,7,8-tetrahydroquinoline-3-carbonitrile

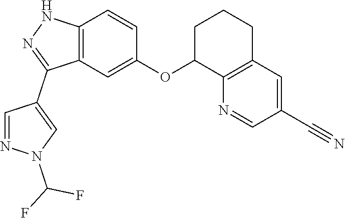 [SMILES: N#Cc1cnc2c(c1)CCCC2Oc1ccc2[nH]nc(-c3cnn(C(F)F)c3)c2c1]

A mixture of 3-bromo-8-((3-(1-(difluoromethyl)-1H-pyrazol-4-yl)-1H-indazol-5-yl)oxy)-5,6,7,8-tetrahydroquinoline (30 mg, 65.18 umol), Zn(CN)₂ (15.31 mg, 130.36 umol), Zn (4.26 mg, 65.18 umol), Pd₂(dba)₃ (11.94 mg, 13.04 umol), and DPPF (7.23 mg, 13.04 umol) in DMA (1 mL) was degassed and purged with N₂ (3×) at 20° C. and then the mixture was stirred at 120° C. for 12 hrs. The reaction mixture was concentrated and purified by preparative-HPLC using Method EJ to afford the title compound (11 mg, 32%) as a white solid TFA salt. MS-ESI (m/z) calc'd for C₂₁H₁₇F₂N₆O [M+H]⁺:407.1. Found 407.1.

### Step 4: 8-((3-(1-(Difluoromethyl)-1H-pyrazol-4-yl)-1H-indazol-5-yl)oxy)-5,6,7,8-tetrahydroquinoline-3-carbonitrile, enantiomer 1 and 2

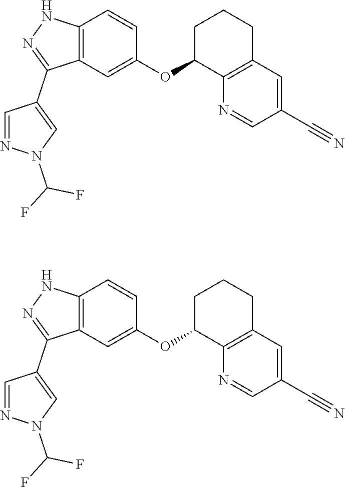 [SMILES: N#Cc1cnc2c(c1)CCC[C@@H]2Oc1ccc2[nH]nc(-c3cnn(C(F)F)c3)c2c1 ; N#Cc1cnc2c(c1)CCC[C@H]2Oc1ccc2[nH]nc(-c3cnn(C(F)F)c3)c2c1]

8-((3-(1-(Difluoromethyl)-1H-pyrazol-4-yl)-1H-indazol-5-yl)oxy)-5,6,7,8-tetrahydroquinoline-3-carbonitrile was subjected to chiral separation using Method EK to afford the two enantiomers. The first eluting fraction was re-purified by preparative-HPLC using Method EL to afford 8-((3-(1-(difluoromethyl)-1H-pyrazol-4-yl)-1H-indazol-5-yl)oxy)-5,6,7,8-tetrahydroquinoline-3-carbonitrile, enantiomer 1 (1.23 mg, 13%) as a yellow solid TFA salt. ¹H NMR (400 MHz, DMSO-d₆) δ 13.06 (br s, 1H), 8.90 (s, 1H), 8.86 (d, J=1.96 Hz, 1H), 8.36 (s, 1H), 8.21 (d, J=1.83 Hz, 1H), 7.71-8.03 (m, 1H), 7.68 (d, J=1.96 Hz, 1H), 7.49 (d, J=8.93 Hz, 1H), 7.15 (dd, J=8.99, 2.14 Hz, 1H), 5.68-5.71 (m, 1H), 2.92-3.01 (m, 1H), 2.77-2.88 (m, 1H), 2.22-2.30 (m, 1H), 1.94-2.02 (m, 2H), 1.73-1.87 (m, 1H). MS-ESI (m/z) calc'd for C₂₁H₁₇F₂N₆O [M+H]⁺:407.1. Found 407.0. A later eluting fraction was also isolated and re-purified by preparative-HPLC using Method EM to afford 8-((3-(1-(difluoromethyl)-1H-pyrazol-4-yl)-1H-indazol-5-yl)oxy)-5,6,7,8-tetrahydroquinoline-3-carbonitrile, enantiomer 2 (1.57 mg, 17%) as a white solid. ¹H NMR (400 MHz, DMSO-d₆) δ 13.06 (br s, 1H), 8.91 (s, 1H), 8.87 (d, J=1.83 Hz, 1H), 8.37 (s, 1H), 8.21 (s, 1H), 7.71-8.03 (m, 1H), 7.69 (d, J=1.96 Hz, 1H), 7.49 (d, J=9.05 Hz, 1H), 7.16 (dd, J=9.05, 2.08 Hz, 1H), 5.67-5.74 (m, 1H), 2.93-3.00 (m, 1H), 2.80-2.88 (m, 1H), 2.21-2.30 (m, 1H), 1.92-2.05 (m, 2H), 1.74-1.85 (m, 1H). MS-ESI (m/z) calc'd for C₂₁H₁₇F₂N₆₀ [M+H]⁺:407.1. Found 407.0.

### Example 142: 8-((3-(Thiazol-5-yl)-1H-indazol-5-yl)amino)-5,6,7,8-tetrahydroquinoline-3-carbonitrile, enantiomer 1 and 2

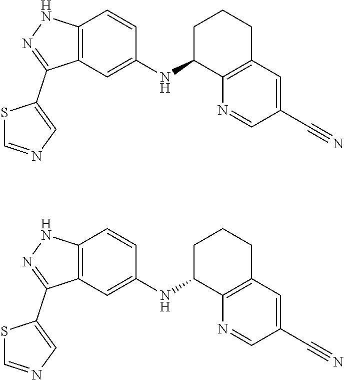 [SMILES: N#Cc1cnc2c(c1)CCC[C@@H]2Nc1ccc2[nH]nc(-c3cncs3)c2c1 ; N#Cc1cnc2c(c1)CCC[C@H]2Nc1ccc2[nH]nc(-c3cncs3)c2c1]

### Step 1: 3-Iodo-1H-indazol-5-amine

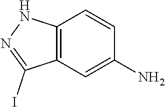 [SMILES: Nc1ccc2[nH]nc(I)c2c1]

To a solution of 3-iodo-5-nitro-1H-indazole (500 mg, 1.73 mmol) in EtOH (15 mL) and H₂O (3 mL) was added Fe (483.04 mg, 8.65 mmol) and NH₄Cl (462.68 mg, 8.65 mmol) at 20° C. The mixture was stirred at 80° C. for 1 hr. The reaction mixture was filtered and the filtrate was diluted with H₂O and extracted with EtOAc (5×). The combined organic layers were dried over Na₂SO₄, filtered and concentrated to give a residue. The residue was purified by flash silica gel chromatography (ISCO; 12 g SepaFlash column) using a 0-49% EtOAc/petroleum ether gradient eluent to afford the title compound (210 mg, 47%) as a brown solid. MS-ESI (m/z) calc'd for C₇H₇IN₃ [M+H]⁺:260.0. Found:259.9.

### Step 2: 8-((3-Iodo-1H-indazol-5-yl)amino)-5,6,7,8-tetrahydroquinoline-3-carbonitrile

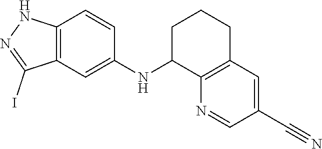 [SMILES: N#Cc1cnc2c(c1)CCCC2Nc1ccc2[nH]nc(I)c2c1]

To a solution of 3-iodo-1H-indazol-5-amine (70 mg, 270.22 umol) and 8-oxo-5,6,7,8-tetrahydroquinoline-3-carbonitrile (46.53 mg, 270.22 umol) in MeOH (2 mL) was added AcOH (16.23 mg, 270.22 umol) to adjust to pH=5 and the mixture was stirred at 50° C. for 1 hr. Then NaBH₃CN (16.98 mg, 270.22 umol) was added and the mixture was stirred at 20° C. for 12 hrs. The reaction mixture was concentrated and purified by preparative-TLC (SiO₂, petroleum ether/EtOAc=1/1, Rf=0.2) to afford the title compound (70 mg, 62%) as a white solid. MS-ESI (m/z) calc'd for C₁₇H₁₅IN₅ [M+H]⁺: 416.0. Found: 416.0.

### Step 3: 8-((3-(Thiazol-5-yl)-1H-indazol-5-yl)amino)-5,6,7,8-tetrahydroquinoline-3-carbonitrile

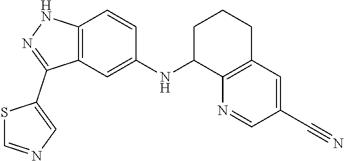 [SMILES: N#Cc1cnc2c(c1)CCCC2Nc1ccc2[nH]nc(-c3cncs3)c2c1]

To a solution of 8-((3-iodo-1H-indazol-5-yl)amino)-5,6,7,8-tetrahydroquinoline-3-carbonitrile (60 mg, 144.50 umol) in DMF (3 mL) were added 5-(dibutyl(pentyl)stannyl)thiazole (54.07 mg, 144.50 umol) and Pd(PPh₃)₂Cl₂ (10.14 mg, 14.45 umol) at 20° C. and the mixture was stirred at 80° C. for 12 hrs under an N₂ atmosphere. The reaction mixture was then concentrated and purified by preparative-HPLC using Method EN to afford the title compound (15 mg, 21%, TFA salt) as a white solid. MS-ESI (m/z) calc'd for C₂₀H₁₇N₆S [M+H]⁺: 373.1. Found: 373.3.

### Step 4: 8-((3-(Thiazol-5-yl)-1H-indazol-5-yl)amino)-5,6,7,8-tetrahydroquinoline-3-carbonitrile, enantiomer 1 and 2

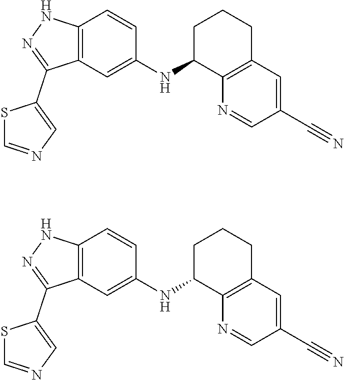 [SMILES: N#Cc1cnc2c(c1)CCC[C@@H]2Nc1ccc2[nH]nc(-c3cncs3)c2c1 ; N#Cc1cnc2c(c1)CCC[C@H]2Nc1ccc2[nH]nc(-c3cncs3)c2c1]

8-((3-(Thiazol-5-yl)-1H-indazol-5-yl)amino)-5,6,7,8-tetrahydroquinoline-3-carbonitrile was subjected to chiral separation using Method EO to afford: 8-((3-(thiazol-5-yl)-1H-indazol-5-yl)amino)-5,6,7,8-tetrahydroquinoline-3-carbonitrile, enantiomer 1 (4.08 mg, 35%) as a white solid. ¹H NMR (400 MHz, DMSO-d₆) δ 12.99 (s, 1H), 9.04 (s, 1H), 8.80 (s, 1H), 8.44 (s, 1H), 8.13 (d, J=1.97 Hz, 1H), 7.35 (d, J=8.99 Hz, 1H), 7.16 (s, 1H), 7.00 (dd, J=1.97, 8.99 Hz, 1H), 5.93 (d, J=7.23 Hz, 1H), 4.81-4.88 (m, 1H), 2.74-2.98 (m, 2H), 1.80-2.11 (m, 4H). MS-ESI (m/z) calc'd for C₂₀H₁₇N₆S [M+H]⁺: 373.1. Found: 373.0. A later eluting fraction was also isolated to afford 8-((3-(thiazol-5-yl)-1H-indazol-5-yl)amino)-5,6,7,8-tetrahydroquinoline-3-carbonitrile, enantiomer 2 (4.42 mg, 38%) as a white solid. ¹H NMR (400 MHz, DMSO-d₆) δ 12.99 (s, 1H), 9.04 (s, 1H), 8.80 (s, 1H), 8.44 (s, 1H), 8.13 (s, 1H), 7.35 (d, J=8.99 Hz, 1H), 7.16 (s, 1H), 7.00 (d, J=8.77 Hz, 1H), 5.94 (d, J=7.23 Hz, 1H), 4.84 (br d, J=6.14 Hz, 1H), 2.76-2.97 (m, 2H), 1.79-2.12 (m, 4H). MS-ESI (m/z) calc'd for C₂₀H₁₇N₆S [M+H]⁺: 373.1. Found: 373.0.

### Example 143: 1-Chloro-5-((3-(oxazol-5-yl)-1H-indazol-5-yl)amino)-5,6,7,8-tetrahydronaphthalene-2-carbonitrile, enantiomer 1 and 2

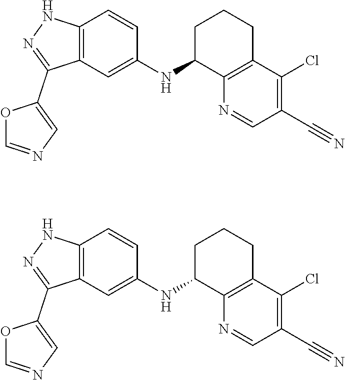 [SMILES: N#Cc1cnc2c(c1Cl)CCC[C@@H]2Nc1ccc2[nH]nc(-c3cnco3)c2c1 ; N#Cc1cnc2c(c1Cl)CCC[C@H]2Nc1ccc2[nH]nc(-c3cnco3)c2c1]

### Step 1: 6-Amino-5-chloro-3,4-dihydronaphthalen-1(2H)-one

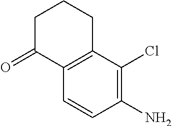 [SMILES: Nc1ccc2c(c1Cl)CCCC2=O]

To a solution of 6-amino-3,4-dihydronaphthalen-1(2H)-one (10 g, 62.03 mmol) in DCM (100 mL) was added NCS (8.28 g, 62.03 mmol) at 0° C. The mixture was then stirred at 20° C. for 12 hrs. The mixture was filtered and the filtrate was evaporated to give a residue. The residue was purified by flash silica gel column chromatography (ISCO; 120 g SepaFlash column) using a 0-15% EtOAc/petroleum ether gradient eluent to afford the title compound (5 g, 41%) as a yellow solid. ¹H NMR (400 MHz, DMSO-d₆) δ 7.61 (d, J=8.6 Hz, 1H), 6.72 (d, J=8.6 Hz, 1H), 2.87 (t, J=6.1 Hz, 2H), 2.45-2.39 (m, 2H), 1.99 (quin, J=6.3 Hz, 2H). MS-ESI (m/z) calc'd for C₁₀H₁₁ClNO [M+H]⁺: 196.1/198.1. Found 196.2/198.2.

### Step 2: 6-Bromo-5-chloro-3,4-dihydronaphthalen-1(2H)-one

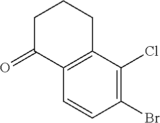 [SMILES: O=C1CCCc2c1ccc(Br)c2Cl]

To a solution of 6-amino-5-chloro-3,4-dihydronaphthalen-1(2H)-one (4 g, 20.45 mmol) and CuBr (8.80 g, 61.34 mmol) in acetonitrile (80 mL) was added tert-butyl nitrite (6.32 g, 61.34 mmol) at 0° C. The mixture was stirred at 20° C. for 12 hrs. The reaction mixture was diluted with H₂O and EtOAc. The mixture was filtered and the filtrate was extracted with EtOAc (3×). The combined organic phase was dried with anhydrous Na₂SO₄, the mixture was filtered and the filtrate was concentrated to give a residue. The residue was purified by flash silica gel column chromatography (ISCO; 40 g SepaFlash column) using a 0-3% EtOAc/petroleum ether gradient eluent to afford the title compound (2.12 g, 40%) as a pale yellow solid. ¹H NMR (400 MHz, CDCl₃): δ 7.84 (d, J=8.5 Hz, 1H), 7.63 (d, J=8.4 Hz, 1H), 3.03-3.15 (m, 2H), 2.57-2.73 (m, 2H), 2.18 ppm (quin, J=6.4 Hz, 2H). MS-ESI (m/z) calc'd for C₁₀H₉BrClO [M+H]⁺: 258.9/260.9. Found 259.1/261.1.

### Step 3: N-(6-Bromo-5-chloro-1,2,3,4-tetrahydronaphthalen-1-yl)-3-(oxazol-5-yl)-1H-indazol-5-amine

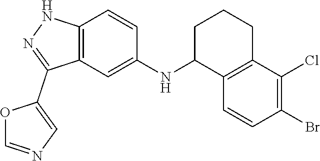 [SMILES: Clc1c(Br)ccc2c1CCCC2Nc1ccc2[nH]nc(-c3cnco3)c2c1]

To a solution of 6-bromo-5-chloro-3,4-dihydronaphthalen-1(2H)-one (200 mg, 770.63 umol) and 3-(oxazol-5-yl)-1H-indazol-5-amine (185.13 mg, 924.76 umol) in MeOH (5 mL) was added AcOH (92.56 mg, 1.54 mmol) to pH=5 at 20° C. The mixture was stirred at 80° C. for 1 hr. Then NaBH₃CN (145.28 mg, 2.31 mmol) was added to the mixture at 20° C. The mixture was stirred at 80° C. for 12 hrs. This procedure was conducted a second time and the residues were combined. The combined reaction mixture was evaporated. The material was purified by flash silica gel column chromatography (ISCO; 12 g SepaFlash column) using a 0-51% EtOAc/petroleum ether gradient eluent to afford the title compound (100 mg, 14%) as a yellow oil. MS-ESI (m/z) calc'd for C₂₀H₁₇BrClN₄O [M+H]⁺: 443.0/445.0. Found 443.1/445.1.

### Step 4: I-Chloro-5-((3-(oxazol-5-yl)-1H-indazol-5-yl)amino)-5,6,7,8-tetrahydronaphthalene-2-carbonitrile

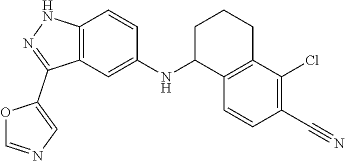 [SMILES: N#Cc1ccc2c(c1Cl)CCCC2Nc1ccc2[nH]nc(-c3cnco3)c2c1]

N-(6-Bromo-5-chloro-1,2,3,4-tetrahydronaphthalen-1-yl)-3-(oxazol-5-yl)-1H-indazol-5-amine (100 mg, 225.37 umol), Zn (10.32 mg, 157.76 umol), Zn(CN)₂ (39.70 mg, 338.05 umol) 1,1-bis(diphenylphosphino)ferrocene (12.49 mg, 22.54 umol) and Pd₂dba₃ (41.27 mg, 45.07 umol) were added to a microwave tube containing DMA (4 mL) at 20° C. The tube was sealed and heated at 100° C. for 2 hrs under microwave irradiation and an N₂ atmosphere. The reaction mixture was combined together with another 50 mg scale reaction before work up. The final mixture was filtered and the filtrate was evaporated to give a residue. The residue was purified by preparative-TLC (SiO₂, petroleum ether/EtOAc=1/2, Rf=0.48) and further purified by preparative-HPLC using Method ER to afford the title compound (8 mg, 6%) as a yellow solid. MS-ESI (m/z) calc'd for C₂₁H₁₇ClN₅O [M+H]⁺: 390.1/392.1. Found 390.2/392.2.

### Step 5: I-Chloro-5-((3-(oxazol-5-yl)-1H-indazol-5-yl)amino)-5,6,7,8-tetrahydronaphthalene-2-carbonitrile, enantiomer 1 and 2

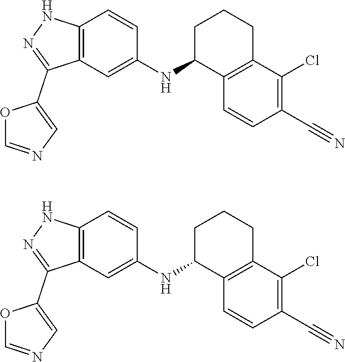 [SMILES: N#Cc1ccc2c(c1Cl)CCC[C@@H]2Nc1ccc2[nH]nc(-c3cnco3)c2c1 ; N#Cc1ccc2c(c1Cl)CCC[C@H]2Nc1ccc2[nH]nc(-c3cnco3)c2c1]

1-Chloro-5-((3-(oxazol-5-yl)-1H-indazol-5-yl)amino)-5,6,7,8-tetrahydronaphthalene-2-carbonitrile was subjected to chiral separation using Method ES to afford 1-chloro-5-((3-(oxazol-5-yl)-1H-indazol-5-yl)amino)-5,6,7,8-tetrahydronaphthalene-2-carbonitrile, enantiomer 1 (4.02 mg, 50%) as a pale yellow solid. ¹H NMR (DMSO-d₆, 400 MHz) δ 13.12 (br s, 1H), 8.45 (s, 1H), 7.75 (br d, J=8.6 Hz, 1H), 7.67 (br s, 1H), 7.52 (br d, J=8.3 Hz, 1H), 7.38 (br d, J=8.8 Hz, 1H), 7.06 (br s, 1H), 6.99 (br d, J=9.0 Hz, 1H), 6.01 (br d, J=9.4 Hz, 1H), 4.86 (br s, 1H), 2.81 (br d, J=15.8 Hz, 2H), 1.87 (br s, 4H). MS-ESI (m/z) calc'd for C₂₁H₁₇ClN₅O [M+H]⁺: 390.1/392.1. Found 390.1/392.1. A later eluting fraction was also isolated to afford 1-chloro-5-((3-(oxazol-5-yl)-1H-indazol-5-yl)amino)-5,6,7,8-tetrahydronaphthalene-2-carbonitrile, enantiomer 2 (3.19 mg, 39%) as a pale yellow solid. ¹H NMR (DMSO-d₆, 400 MHz) δ 13.04-13.19 (m, 1H), 8.45 (d, J=2.0 Hz, 1H), 7.74 (d, J=7.9 Hz, 1H), 7.67 (d, J=2.0 Hz, 1H), 7.52 (d, J=8.6 Hz, 1H), 7.35-7.43 (m, 1H), 7.05 (s, 1H), 6.98 (br d, J=8.8 Hz, 1H), 6.01 (br d, J=9.4 Hz, 1H), 4.88 (br s, 1H), 2.74-2.88 (m, 2H), 1.79-2.01 ppm (m, 4H). MS-ESI (m/z) calc'd for C₂₁H₁₇ClN₅O [M+H]⁺: 390.1/392.1. Found 390.1/392.1.

### Example 144: 4-Chloro-8-((3-(oxazol-5-yl)-1H-indazol-5-yl)amino)-5,6,7,8-tetrahydroquinoline-3-carbonitrile, enantiomer 1 and 2

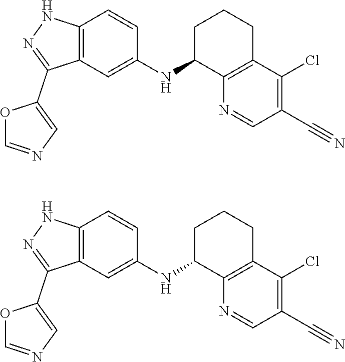 [SMILES: N#Cc1cnc2c(c1Cl)CCC[C@@H]2Nc1ccc2[nH]nc(-c3cnco3)c2c1 ; N#Cc1cnc2c(c1Cl)CCC[C@H]2Nc1ccc2[nH]nc(-c3cnco3)c2c1]

### Step 1: 3-(4,4,5,5-tetramethyl-1,3,2-dioxaborolan-2-yl)-5,6,7,8-tetrahydroquinoline

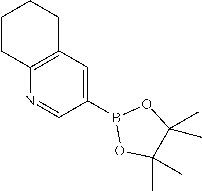 [SMILES: CC1(C)OB(c2cnc3c(c2)CCCC3)OC1(C)C]

A mixture of 5,6,7,8-tetrahydroquinoline (19 g, 142.65 mmol), bis(pinacolato)diboron (36.23 g, 142.65 mmol), (1,5-cyclooctadiene)(methoxy)iridium(I) dimer (2.84 g, 4.28 mmol), 4,4′-di-tert-butyl-2,2′-dipyridyl (2.30 g, 8.56 mmol) in THF (180 mL) was degassed and purged with N₂ (3×) at 25° C. and then the mixture was stirred at 75° C. for 12 hrs under an N₂ atmosphere. The reaction mixture was then concentrated to afford the title compound (36 g, 97%) as a black solid, which was used without further purification. MS-ESI (m/z) calc'd for C₁₅H₂₃BNO₂ [M+H]⁺: 260.2. Found: 260.3.

### Step 2: 3-Bromo-5,6,7,8-tetrahydroquinoline

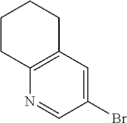 [SMILES: Brc1cnc2c(c1)CCCC2]

To a solution of 3-(4,4,5,5-tetramethyl-1,3,2-dioxaborolan-2-yl)-5,6,7,8-tetrahydroquinoline (36 g, 138.92 mmol) in MeOH (250 mL) was added a solution of CuBr₂ (99.29 g, 444.53 mmol) in H₂O (250 mL) at 25° C. The mixture was stirred at 75° C. for 2 hrs. The reaction mixture was concentrated to remove MeOH. The aqueous solution was basified with NH₃.H₂O to pH=8 and extracted with EtOAc (4×). The combined organic layers were dried over Na₂SO₄, filtered, and concentrated under reduced pressure to give a residue. The residue was purified by flash silica gel chromatography (ISCO; 80 g SepaFlash Silica Flash Column) using a 0-3% EtOAc/petroleum ether gradient eluent to afford the title compound (4 g, 13%) as a colorless oil. ¹H NMR (400 MHz, DMSO-d₆) δ 8.40 (d, J=2.13 Hz, 1H) 7.46-7.55 (d, 1H) 2.87 (t, J=6.38 Hz, 2H) 2.76 (t, J=6.32 Hz, 2H) 1.85-1.93 (m, 2H) 1.76-1.84 (m, 2H). MS-ESI (m/z) calc'd for C₉H₁₁BrN [M+H]⁺:212.0/214.0. Found: 212.2/214.1.

### Step 3: 3-Bromo-5,6,7,8-tetrahydroquinoline 1-oxide

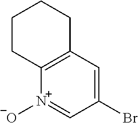 [SMILES: [O-][n+]1cc(Br)cc2c1CCCC2]

To a solution of 3-bromo-5,6,7,8-tetrahydroquinoline (8 g, 37.72 mmol) in DCM (160 mL) was added m-CPBA (16.27 g, 75.44 mmol) at 20° C. The mixture was stirred at 40° C. for 2 hrs. The reaction mixture was quenched with a 10% aqueous solution of Na₂SO₃ and extracted with EtOAc (3×). The combined organic layers were dried over Na₂SO₄, filtered, and concentrated to afford the title compound (8 g, 93%) as a white solid. MS-ESI (m/z) calc'd for C₉H₁₁BrNO [M+H]⁺:228.0/230.0. Found 228.1/230.1.

### Step 4: 3-Bromo-4-nitro-5,6,7,8-tetrahydroquinoline 1-oxide

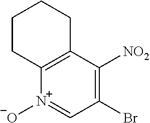 [SMILES: O=[N+]([O-])c1c(Br)c[n+]([O-])c2c1CCCC2]

To a solution of 3-bromo-5,6,7,8-tetrahydroquinoline 1-oxide (9.2 g, 40.34 mmol) in conc. H₂SO₄ (30 mL) was added HNO₃ (23.34 g, 363.02 mmol) slowly at 0° C. The mixture was stirred at 20° C. for 0.5 hr and then warmed to 90° C. and stirred for 4 hrs. The mixture was adjusted to pH=8 with saturated aqueous NaHCO₃ at 0° C. Then it was extracted with EtOAc (3×). The combined organic layers were dried over Na₂SO₄, filtered, and concentrated under reduced pressure to afford the title compound (5 g, 45%) as a yellow solid. MS-ESI (m/z) calc'd for C₉H₁₀BrN₂O₃ [M+H]⁺:273.0/275.0. Found 273.1/275.1.

### Step 5: 3-Bromo-4-chloro-5,6,7,8-tetrahydroquinoline 1-oxide

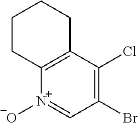 [SMILES: [O-][n+]1cc(Br)c(Cl)c2c1CCCC2]

A solution of 3-bromo-4-nitro-5,6,7,8-tetrahydroquinoline 1-oxide (5 g, 18.31 mmol) in HCl (1.80 g, 18.31 mmol) was stirred at 90° C. for 12 hrs. The mixture was adjusted to pH=8 with 2 M NaOH and extracted with EtOAc (5×). The combined organic layers were dried over Na₂SO₄, filtered, and concentrated to afford the title compound (4.1 g, 85%) as a yellow solid. ¹H NMR (400 MHz, MeOD) δ 8.58 (s, 1H), 2.83-2.91 (m, 4H), 1.82-1.90 (m, 4H). MS-ESI (m/z) calc'd for C₉H₁₀BrClNO [M+H]⁺:262.0/264.0. Found:262.1/264.1.

### Step 6: 3-Bromo-4-chloro-5,6,7,8-tetrahydroquinolin-8-ol

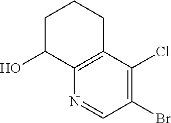 [SMILES: OC1CCCc2c1ncc(Br)c2Cl]

A solution of 3-bromo-4-chloro-5,6,7,8-tetrahydroquinoline 1-oxide (4.1 g, 15.62 mmol) in TFA (30 mL) was stirred at 50° C. for 12 hrs. The reaction mixture was concentrated to give a residue. The residue was diluted with 2 M NaOH aqueous solution to adjust pH=10 and stirred at 25° C. for 1 hr, then it was extracted with EtOAc (3×). The combined organic layers were dried over Na₂SO₄, filtered, and concentrated to give a residue. The residue was purified by flash silica gel chromatography (ISCO; 40 g SepaFlash column) using a 0-11% EtOAc/petroleum ether gradient eluent to afford the title compound (2 g, 49%). ¹H NMR (400 MHz, DMSO-d₆) δ 8.57 (br s, 1H) 4.62-4.74 (m, 1H) 3.77-3.88 (m, 1H) 2.78-2.95 (m, 2H) 2.21-2.32 (m, 1H) 2.04-2.14 (m, 1H) 1.78-1.89 (m, 2H). MS-ESI (m/z) calc'd for C₉H₁₀BrClNO [M+H]⁺:262.0/264.0. Found:262.1/264.1.

### Step 7: 3-Bromo-4-chloro-6,7-dihydroquinolin-8(5H)-one

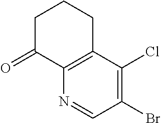 [SMILES: O=C1CCCc2c1ncc(Br)c2Cl]

To a solution of 3-bromo-4-chloro-5,6,7,8-tetrahydroquinolin-8-ol (400 mg, 1.52 mmol) in DCM (20 mL) was added Dess-Martin periodinane (1.29 g, 3.05 mmol). The mixture was stirred at 20° C. for 2 hrs. The mixture was basified with saturated aqueous Na₂CO₃ to adjust pH=8, the mixture was filtered and the filtrate was extracted with DCM (4×). The combined organic layers were dried over Na₂SO₄, filtered and concentrated to afford the title compound (392 mg, 99%) as a yellow solid. MS-ESI (m/z) calc'd for C₉H₈BrClNO [M+H]⁺:260.0/262.0. Found:260.1/262.1.

### Step 8: 3-Bromo-4-chloro-N-(3-(oxazol-5-yl)-1H-indazol-5-yl)-5,6,7,8-tetrahydroquinolin-8 amine

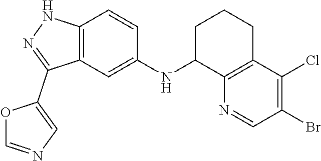 [SMILES: Clc1c(Br)cnc2c1CCCC2Nc1ccc2[nH]nc(-c3cnco3)c2c1]

To a solution of 3-(oxazol-5-yl)-1H-indazol-5-amine (308.30 mg, 1.54 mmol), 3-bromo-4-chloro-6,7-dihydroquinolin-8(5H)-one (400 mg, 1.54 mmol) in MeOH (20 mL) was added AcOH (277.43 mg, 4.62 mmol) to adjust to pH=5. The mixture was stirred at 25° C. for 1 hr, then NaBH₃CN (580.64 mg, 9.24 mmol) was added and the mixture was stirred at 25° C. for 12 hrs. A solid formed that was collected by filtration. The obtained solid was washed with H₂O (3×) and dried to afford 120 mg of product. Additional product was obtained by concentrating the filtrate to a residue and purifying the residue by preparative HPLC using Method ET to give an additional 40 mg of product. These were combined to afford the title compound (160 mg, 23%) as a yellow solid. ¹H NMR (400 MHz, DMSO-d₆) δ 13.09 (br s, 1H), 8.68 (s, 1H), 8.48 (s, 1H), 7.66 (s, 1H), 7.36 (br d, J=8.75 Hz, 1H), 7.08 (br s, 1H), 6.98 (br d, J=8.50 Hz, 1H), 5.92 (br d, J=7.25 Hz, 1H), 4.74 (br s, 1H), 2.87-3.00 (m, 1H), 2.72-2.82 (m, 1H), 1.85-2.06 (m, 4H) MS-ESI (m/z) calc'd for C₁₉H₁₆BrClN₅O [M+H]⁺:444.0/446.0. Found:444.1/446.1.

### Step 9: 4-Chloro-8-((3-(oxazol-5-yl)-1H-indazol-5-yl)amino)-5,6,7,8-tetrahydroquinoline-3-carbonitrile

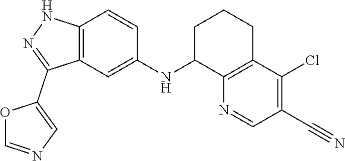 [SMILES: N#Cc1cnc2c(c1Cl)CCCC2Nc1ccc2[nH]nc(-c3cnco3)c2c1]

A mixture of 3-bromo-4-choro-N-(3-oxazol-5-yl-1H-indazol-5-yl)-5,6,7,8-tetrahydroquinolin-8-amine (10 mg, 22.49 umol), Zn(CN)₂ (1.06 mg, 8.99 umol), and Pd(PPh₃)₄ (2.60 mg, 2.25 umol) in DMF (0.5 mL) was degassed and purged with N₂ (3×) at 20° C. The mixture was then stirred under an N₂ atmosphere at 120° C. using microwave irradiation for 5 minutes. This procedure was conducted five times and the residues were combined. The final mixture was diluted with H₂O and extracted with EtOAc (3×). The combined organic layers were washed with brine (2×), dried over Na₂SO₄, filtered, and concentrated under reduced pressure to give a residue. The residue was purified by preparative-TLC (100% EtOAc, Rf=0.53) and further purified by preparative-HPLC using Method EU to afford the title compound (6 mg, 10%) as a yellow solid TFA salt. MS-ESI (m/z) calc'd for C₂₀H₁₆ClN₆O [M+H]⁺:391.1. Found: 391.2.

### Step 10: 4-Chloro-8-((3-(oxazol-5-yl)-1H-indazol-5-yl)amino)-5,6,7,8-tetrahydroquinoline-3-carbonitrile, enantiomer 1 and 2

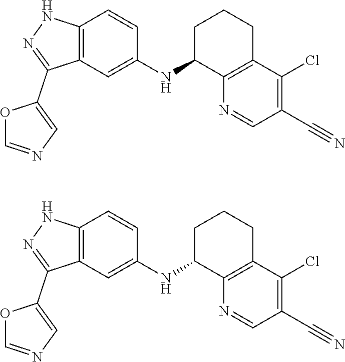 [SMILES: N#Cc1cnc2c(c1Cl)CCC[C@@H]2Nc1ccc2[nH]nc(-c3cnco3)c2c1 ; N#Cc1cnc2c(c1Cl)CCC[C@H]2Nc1ccc2[nH]nc(-c3cnco3)c2c1]

4-Chloro-8-((3-(oxazol-5-yl)-1H-indazol-5-yl)amino)-5,6,7,8-tetrahydroquinoline-3-carbonitrile was subjected to chiral separation using Method EV to afford 4-chloro-8-((3-(oxazol-5-yl)-1H-indazol-5-yl)amino)-5,6,7,8-tetrahydroquinoline-3-carbonitrile, enantiomer 1 (2.45 mg, 27%)¹H NMR (400 MHz, DMSO-d₆) δ 13.09 (s, 1H), 8.90 (s, 1H), 8.47 (s, 1H), 7.66 (s, 1H), 7.36 (d, J=9.05 Hz, 1H), 7.10 (s, 1H), 6.97 (dd, J=8.99, 1.89 Hz, 1H), 5.98 (d, J=7.82 Hz, 1H), 4.79-4.89 (m, 1H), 2.87-2.96 (m, 1H), 2.76-2.83 (m, 1H), 1.88-2.04 (m, 4H). MS-ESI (m/z) calc'd for C₂₀H₁₆ClN₆O [M+H]⁺: 391.1. Found: 391.1. A later eluting fraction was also isolated to afford 4-chloro-8-((3-(oxazol-5-yl)-1H-indazol-5-yl)amino)-5,6,7,8-tetrahydroquinoline-3-carbonitrile, enantiomer 2 (1.32 mg, 14%). ¹H NMR (400 MHz, DMSO-d₆) δ 13.09 (s, 1H), 8.90 (s, 1H), 8.47 (s, 1H), 7.66 (s, 1H), 7.36 (d, J=8.88 Hz, 1H), 7.10 (s, 1H), 6.97 (dd, J=8.94, 1.94 Hz, 1H), 5.98 (d, J=7.75 Hz, 1H), 4.77-4.91 (m, 1H), 2.88-2.95 (m, 1H), 2.76-2.83 (m, 1H), 1.90-2.03 (m, 4H). MS-ESI (m/z) calc'd for C₂₀H₁₆ClN₆O [M+H]⁺:391.1. Found: 391.1.

### Example 145: 8-((3-(Oxazol-5-yl)-1H-indazol-5-yl)amino)-4-(trifluoromethyl)-5,6,7,8-tetrahydroquinoline-3-carbonitrile, enantiomer 1 and 2

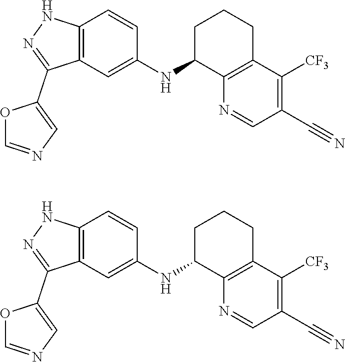 [SMILES: N#Cc1cnc2c(c1C(F)(F)F)CCC[C@@H]2Nc1ccc2[nH]nc(-c3cnco3)c2c1 ; N#Cc1cnc2c(c1C(F)(F)F)CCC[C@H]2Nc1ccc2[nH]nc(-c3cnco3)c2c1]

### Step 1: 2-(2,2,2-Trifluoroacetyl)cyclohexanone

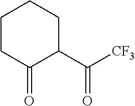 [SMILES: O=C1CCCCC1C(=O)C(F)(F)F]

To a solution of cyclohexanone (10 g, 101.89 mmol) in THF (200 mL) was added LDA (2 M, 50.95 mL) slowly at −70° C. under an N₂ atmosphere and the mixture was stirred for 10 minutes. Ethyl 2,2,2-trifluoroacetate (14.48 g, 101.89 mmol) was then added slowly at −70° C. The resulting mixture was stirred at 20° C. for 2 hrs under an N₂ atmosphere. The reaction mixture was quenched by addition of 1 M HCl at −70° C. and extracted with EtOAc (3×). The combined organic layers were dried over Na₂SO₄, filtered, and concentrated to afford the title compound (16 g, 80%) as a yellow oil. MS-ESI (m/z) calc'd for C₈H₈F₃₀₂ [M−H]⁻: 193.1. Found:193.0.

### Step 2: 2-Oxo-4-(trifluoromethyl)-1,2,5,6,7,8-hexahydroquinoline-3-carbonitrile

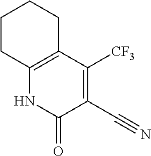 [SMILES: N#Cc1c(C(F)(F)F)c2c([nH]c1=O)CCCC2]

To a solution of 2-(2,2,2-trifluoroacetyl)cyclohexanone (16 g, 82.41 mmol) in propan-2-ol (30 mL) were added KF (957.55 mg, 16.48 mmol) and 2-cyanoacetamide (9.01 g, 107.13 mmol) at 20° C. The mixture was stirred at 80° C. for 12 hrs. The reaction mixture was diluted with H₂O and filtered. The solid was collected, washed with butanol, and dried under vacuum to afford the title compound (11 g, 55%) as a white solid. ¹H NMR (400 MHz, DMSO-d₆) δ 12.88 (br s, 1H), 2.28-2.40 (m, 4H), 1.49-1.53 (m, 4H). MS-ESI (m/z) calc'd for C₁₁H₁₀F₃N₂O [M+H]⁺:243.1. Found: 243.0.

### Step 3:2-Chloro-4-(trifluoromethyl)-5,6,7,8-tetrahydroquinoline-3-carbonitrile

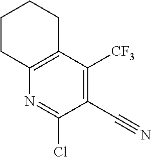 [SMILES: N#Cc1c(Cl)nc2c(c1C(F)(F)F)CCCC2]

A mixture of 2-oxo-4-(trifluoromethyl)-1,2,5,6,7,8-hexahydroquinoline-3-carbonitrile (7.5 g, 30.97 mmol) in POCl₃ (47.48 g, 309.67 mmol) was stirred at 100° C. for 12 hrs. The reaction mixture was then concentrated to give a residue. The residue was quenched with saturated aqueous NaHCO₃ and extracted with EtOAc (4×). The combined organic phases were dried with anhydrous Na₂SO₄, filtered, and concentrated to afford the title compound (7.3 g, 90%) as a pale yellow solid. ¹H NMR (400 MHz, DMSO-d₆) δ 3.00 (br t, J=6.14 Hz, 2H), 2.88 (br s, 2H), 1.74-1.87 (m, 4H). MS-ESI (m/z) calc'd for C₁₁H₉ClF₃N₂ [M+H]⁺:261.0/263.0. Found:261.2/263.2.

### Step 4: 4-(Trifluoromethyl)-5,6,7,8-tetrahydroquinoline-3-carbonitrile

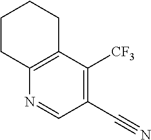 [SMILES: N#Cc1cnc2c(c1C(F)(F)F)CCCC2]

To a solution of 2-chloro-4-(trifluoromethyl)-5,6,7,8-tetrahydroquinoline-3-carbonitrile (7 g, 26.86 mmol) in MeOH (40 mL) was added wet 10% Pd/C (7 g) and ammonium formate (5.08 g, 80.57 mmol) at 20° C. The mixture was stirred at 60° C. for 5 hrs. The mixture was filtered and the filtrate was concentrated to give a residue that was purified by flash silica gel chromatography (ISCO; 20 g SepaFlash column) using a 0-8% EtOAc/petroleum ether gradient eluent to afford the title compound (3.2 g, 53%) as a yellow solid. ¹H NMR (400 MHz, DMSO-d₆) δ 8.96 (s, 1H), 3.01 (t, J=6.36 Hz, 2H), 2.85-2.93 (m, 2H), 1.74-1.89 (m, 4H). MS-ESI (m/z) calc'd for C₁₁H₁₀F₃N₂ [M+H]⁺:227.1. Found:227.2.

### Step 5: 3-Cyano-4-(trifluoromethyl)-5,6,7,8-tetrahydroquinoline 1-oxide

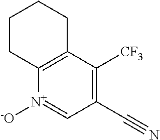

To a solution of 4-(trifluoromethyl)-5,6,7,8-tetrahydroquinoline-3-carbonitrile (1 g, 4.42 mmol) in DCM (10 mL) was added m-CPBA (1.80 g, 8.84 mmol) at 20° C. The mixture was then stirred at 40° C. for 4 hrs. The reaction mixture was quenched by addition of 10% aqueous Na₂SO₃ at 0° C. and basified with saturated aqueous NaHCO₃ to pH=8. The mixture was extracted with DCM (4×) and the combined organic layers were dried over Na₂SO₄, filtered, and concentrated to afford the title compound (0.98 g, 92%) as a pale yellow solid. MS-ESI (m/z) calc'd for C₁₁H₁₀F₃N₂O [M+H]⁺:243.1. Found:243.2.

### Step 6: 8-Hydroxy-4-(trifluoromethyl)-5,6,7,8-tetrahydroquinoline-3-carbonitrile

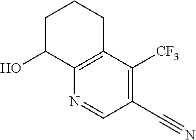 [SMILES: N#Cc1cnc2c(c1C(F)(F)F)CCCC2O]

To a solution of 3-cyano-4-(trifluoromethyl)-5,6,7,8-tetrahydroquinoline 1-oxide (1 g, 4.13 mmol) in DCM (6 mL) was added TFAA (5.20 g, 24.77 mmol) at 20° C. The mixture was stirred at 20° C. for 12 hrs. The reaction mixture was poured into 2 M aqueous NaOH and stirred for 5 minutes. Then it was extracted with EtOAc (4×) and the combined organic phases were dried over anhydrous Na₂SO₄, filtered, and concentrated to give a residue. The residue was purified by flash silica gel chromatography (ISCO; 12 g SepaFlash column) using a 0-22% EtOAc/petroleum ether gradient eluent to afford the title compound (418 mg, 42%) as a yellow solid. ¹H NMR (400 MHz, DMSO-d₆) δ 9.13 (s, 1H), 5.71 (br s, 1H), 4.68 (t, J=4.28 Hz, 1H), 2.92-3.05 (m, 1H), 2.79-2.91 (m, 1H), 1.72-1.97 (m, 4H). MS-ESI (m/z) calc'd for C₁₁H₁₀F₃N₂O [M+H]⁺:243.1. Found:243.1.

### Step 7: 8-Oxo-4-(trifluoromethyl)-5,6,7,8-tetrahydroquinoline-3-carbonitrile

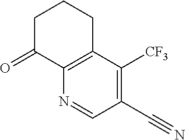 [SMILES: N#Cc1cnc2c(c1C(F)(F)F)CCCC2=O]

To a solution of 8-hydroxy-4-(trifluoromethyl)-5,6,7,8-tetrahydroquinoline-3-carbonitrile (150 mg, 619.33 umol) in DCM (2.5 mL) was added Dess-Martin periodinane (315.22 mg, 743.20 umol) at 20° C. The mixture was stirred at 20° C. for 2 hrs. The reaction mixture was adjusted to pH=8 by addition of saturated aqueous NaHCO₃ and extracted with EtOAc (3×). The combined organic phases were dried over anhydrous Na₂SO₄, filtered, and concentrated to afford the title compound (85 mg, 57%) as a yellow solid. ¹H NMR (400 MHz, DMSO-d₆) δ 9.29 (s, 1H), 3.16 (br d, J=4.82 Hz, 2H), 2.81 (t, J=6.58 Hz, 2H), 2.14 (quin, J=6.30 Hz, 2H). MS-ESI (m/z) calc'd for C₁₁H₈F₃N₂O [M+H]⁺:241.1. Found:241.1.

### Step 8: 8-((3-(Oxazol-5-yl)-1H-indazol-5-yl)amino)-4-(trifluoromethyl)-5,6,7,8-tetrahydroquinoline-3-carbonitrile

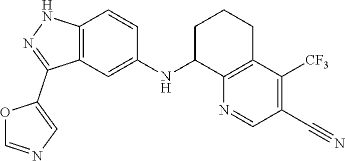 [SMILES: N#Cc1cnc2c(c1C(F)(F)F)CCCC2Nc1ccc2[nH]nc(-c3cnco3)c2c1]

To a solution of 8-oxo-4-(trifluoromethyl)-5,6,7,8-tetrahydroquinoline-3-carbonitrile (150 mg, 624.53 umol) and 3-(oxazol-5-yl)-1H-indazol-5-amine (62.51 mg, 312.26 umol) in MeOH (2 mL) was added AcOH (1.88 mg, 31.23 umol) to adjust to pH=5. The mixture was stirred at 20° C. for 2 hrs and then NaBH₃CN (58.87 mg, 936.79 umol) was added. The mixture was stirred at 20° C. for an additional 2 hrs and filtered. The filtrate was concentrated and purified by preparative-HPLC using Method EW to afford the title compound (10 mg, 6%) as a solid pale yellow TFA salt. MS-ESI (m/z) calc'd for C₂₁H₁₆F₃N₆O [M+H]⁺:425.1. Found:425.3.

### Step 9: 8-((3-(Oxazol-5-yl)-1H-indazol-5-yl)amino)-4-(trifluoromethyl)-5,6,7,8-tetrahydroquinoline-3-carbonitrile, enantiomer 1 and 2

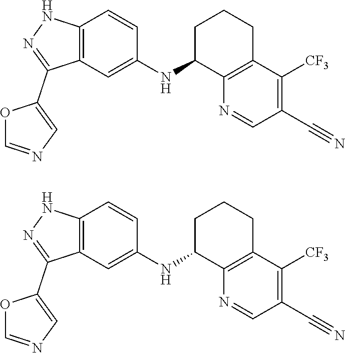 [SMILES: N#Cc1cnc2c(c1C(F)(F)F)CCC[C@@H]2Nc1ccc2[nH]nc(-c3cnco3)c2c1 ; N#Cc1cnc2c(c1C(F)(F)F)CCC[C@H]2Nc1ccc2[nH]nc(-c3cnco3)c2c1]

8-((3-(Oxazol-5-yl)-1H-indazol-5-yl)amino)-4-(trifluoromethyl)-5,6,7,8-tetrahydroquinoline-3-carbonitrile was subjected to chiral separation using Method EX to afford 8-((3-(oxazol-5-yl)-1H-indazol-5-yl)amino)-4-(trifluoromethyl)-5,6,7,8-tetrahydroquinoline-3-carbonitrile, enantiomer 1 (2.94 mg, 29%) as a pale yellow solid. ¹H NMR (400 MHz, DMSO-d₆) δ 13.10 (s, 1H), 9.09 (s, 1H), 8.47 (s, 1H), 7.67 (s, 1H), 7.36 (d, J=9.04 Hz, 1H), 7.09 (s, 1H), 6.96 (br d, J=9.04 Hz, 1H), 6.06 (d, J=8.16 Hz, 1H), 4.92 (br d, J=6.84 Hz, 1H), 2.90-3.06 (m, 2H), 1.84-2.12 (m, 4H). MS-ESI (m/z) calc'd for C₂₁H₁₆F₃N₆O [M+H]⁺: 425.1. Found: 425.1. A later eluting fraction was also isolated to afford 8-((3-(oxazol-5-yl)-1H-indazol-5-yl)amino)-4-(trifluoromethyl)-5,6,7,8-tetrahydroquinoline-3-carbonitrile, enantiomer 2 (3.25 mg, 32%) as a pale yellow solid. ¹H NMR (400 MHz, DMSO-d₆) δ 13.10 (s, 1H), 9.10 (s, 1H), 8.47 (s, 1H), 7.66 (s, 1H), 7.36 (d, J=8.82 Hz, 1H), 7.10 (s, 1H), 6.94-7.01 (m, 1H), 6.06 (d, J=7.72 Hz, 1H), 4.92 (br d, J=7.28 Hz, 1H), 2.90-3.11 (m, 2H), 1.87-2.09 (m, 4H). MS-ESI (m/z) calc'd for C₂₁H₁₆F₃N₆₀ [M+H]⁺: 425.1. Found: 425.1.

### Example 146: 4-Methoxy-8-((3-(oxazol-5-yl)-1H-indazol-5-yl)amino)-5,6,7,8-tetrahydroquinoline-3-carbonitrile, enantiomer 1 and 2

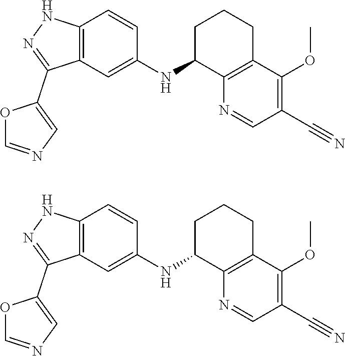 [SMILES: COc1c(C#N)cnc2c1CCC[C@@H]2Nc1ccc2[nH]nc(-c3cnco3)c2c1 ; COc1c(C#N)cnc2c1CCC[C@H]2Nc1ccc2[nH]nc(-c3cnco3)c2c1]

### Step 1: 5,6,7,8-Tetrahydroquinoline 1-oxide

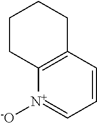

To a solution of 5,6,7,8-tetrahydroquinoline (10 g, 75.08 mmol) in CH₂Cl₂ (200 mL) was added m-CPBA (18.29 g, 90.10 mmol) at 25° C. The mixture was stirred at 25° C. for 12 hrs. The mixture was then diluted with 10% aqueous Na₂SO₃ at 0° C. and stirred at 25° C. for 0.5 hr. The mixture was extracted with CH₂Cl₂ (3×) and the combined organic layers were dried over Na₂SO₄, filtered, and concentrated under reduced pressure to afford the title compound (11 g, 98%) as yellow oil. MS-ESI (m/z) calc'd for C₉H₁₂NO [M+H]⁺: 150.1. Found 150.2.

### Step 2: 4-Nitro-5,6,7,8-tetrahydroquinoline 1-oxide

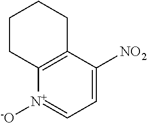

To a solution of 5,6,7,8-tetrahydroquinoline 1-oxide (22 g, 147.46 mmol) in conc. H₂SO₄ (44 mL) was added HNO₃ (88 mL, 1.92 mol) in conc. H₂SO₄ (44 mL) at 0° C. The mixture was stirred at 0° C. for 0.5 hr; then it was stirred at 25° C. for 12 hrs. The mixture was added to a 2 M NaOH solution to adjust to pH=10 and extracted with EtOAc (3×). The combined organic layers were dried over Na₂SO₄, filtered, and concentrated under reduced pressure to give a residue. The residue was purified by flash silica gel column chromatography (ISCO; 300 g SepaFlash column) using a 0-37% EtOAc/petroleum ether gradient eluent to afford the title compound (3 g, 10%) as a yellow solid. ¹H NMR (400 MHz, DMSO-d₆) δ ppm 8.36 (d, J=7.09 Hz, 1H), 7.97 (d, J=7.09 Hz, 1H), 2.99 (t, J=6.05 Hz, 2H), 2.76 (t, J=6.42 Hz, 2H), 1.78-1.87 (m, 2H), 1.64-1.74 (m, 2H). MS-ESI (m/z) calc'd for C₉H₁₁N₂O₃ [M+H]⁺: 195.1. Found 195.2.

### Step 3: 4-Methoxy-5,6,7,8-tetrahydroquinoline 1-oxide

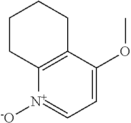

To a solution of NaOMe (8.07 g, 149.34 mmol) in MeOH (200 mL) was added 4-nitro-5,6,7,8-tetrahydroquinoline 1-oxide (14.5 g, 74.67 mmol) at 25° C. The mixture was stirred at 70° C. for 0.5 hr. The mixture was concentrated to give a residue that was diluted with EtOAc and filtered. The solid was washed with EtOAc (2×) and the filtrate was collected and evaporated to dryness to afford the title compound (3.1 g, 23%) as a yellow oil. ¹H NMR (400 MHz, DMSO-d₆) δ 8.12 (d, J=7.21 Hz, 1H), 6.94 (d, J=7.21 Hz, 1H), 3.85 (s, 3H), 2.71 (br t, J=6.17 Hz, 2H), 2.54-2.59 (m, 2H), 1.73-1.78 (m, 2H), 1.63-1.70 (m, 2H). MS-ESI (m/z) calc'd for C₁₀H₁₄NO₂ [M+H]⁺: 180.1. Found 180.2.

### Step 4: 3-Bromo-4-methoxy-5,6,7,8-tetrahydroquinoline 1-oxide

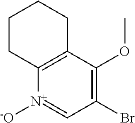

To a solution of 4-methoxy-5,6,7,8-tetrahydroquinoline 1-oxide (1.45 g, 8.09 mmol) in TFA (15 mL) and H₂SO₄ (22 mL) was added NBS (2.88 g, 16.18 mmol) at 25° C. The mixture was stirred at 25° C. for 5 hrs. This procedure was conducted a second time and the residues were combined. The pH of the final mixture was brought to pH=8 with 2 M NaOH and extracted with EtOAc (3×). The combined organic layers were dried over Na₂SO₄, filtered, and concentrated under reduced pressure to afford the title compound (3.3 g, 78%) as a red oil. ¹H NMR (400 MHz, MeOD) δ 8.37 (s, 1H), 3.82 (s, 3H), 2.71-2.76 (m, 4H), 1.76-1.84 (m, 2H), 1.64-1.72 (m, 2H). MS-ESI (m/z) calc'd for C₁₀H₁₃BrNO₂ [M+H]⁺: 258.0/260.0. Found 258.0/260.0.

### Step 5: 3-Bromo-4-methoxy-5,6,7,8-tetrahydroquinolin-8-ol

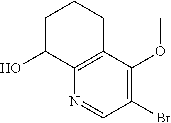

A solution of 3-bromo-4-methoxy-5,6,7,8-tetrahydroquinoline 1-oxide (1 g, 3.87 mmol) in Ac₂O (13.08 g, 128.12 mmol) was stirred at 100° C. for 1 hr. The mixture was then concentrated to give a residue was added to 2 M NaOH (24 mL) at 0° C. and stirred at 25° C. for 2 hrs. The mixture was extracted with EtOAc (3×) and the combined organic phases were dried over anhydrous Na₂SO₄, filtered, and concentrated to give a residue. The residue was purified by flash silica gel column chromatography (ISCO; 4 g SepaFlash column) using a 0-14% EtOAc/petroleum ether gradient eluent to afford the title compound (180 mg, 18%) as a yellow solid. ¹H NMR (400 MHz, MeOD) δ 8.52 (s, 1H), 4.68 (t, J=4.75 Hz, 1H), 3.93 (s, 3H), 2.88-2.97 (m, 1H), 2.70-2.79 (m, 1H), 1.93-2.02 (m, 3H), 1.76-1.86 (m, 1H). MS-ESI (m/z) calc'd for C₁₀H₁₃BrNO₂ [M+H]⁺: 258.0/260.0. Found 258.0/260.0.

### Step 6: 3-Bromo-4-methoxy-6,7-dihydroquinolin-8(5H)-one

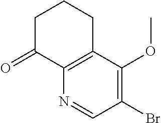

To a solution of 3-bromo-4-methoxy-5,6,7,8-tetrahydroquinolin-8-ol (180 mg, 697.37 umol) in CH₂Cl₂ (3 mL) was added Dess-Martin periodinane (1.18 g, 2.79 mmol) at 25° C. The mixture was stirred at 25° C. for 12 hrs. The reaction mixture was diluted with a NaHCO₃ solution to pH=8 and stirred at 25° C. for 1 hr. The mixture was extracted with EtOAc (3×). The combined organic layers were dried over Na₂SO₄, filtered, and evaporated to dryness to afford the title compound (172.9 mg, 96%) as a yellow solid. MS-ESI (m/z) calc'd for C₁₀H₁₁BrNO₂ [M+H]⁺: 256.0/258.0. Found 256.1/258.1.

### Step 7: 3-Bromo-4-methoxy-N-(3-(oxazol-5-yl)-1H-indazol-5-yl)-5,6,7,8-tetrahydroquinolin-8-amine

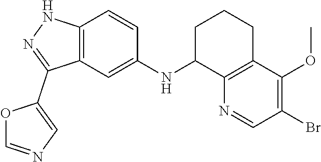 [SMILES: COc1c(Br)cnc2c1CCCC2Nc1ccc2[nH]nc(-c3cnco3)c2c1]

To a solution of 3-bromo-4-methoxy-6,7-dihydroquinolin-8(5H)-one (190 mg, 741.91 umol) in MeOH (5 mL) was added 3-(oxazol-5-yl)-1H-indazol-5-amine (148.53 mg, 741.91 umol) and AcOH (89.10 mg, 1.48 mmol) at 25° C. The mixture was stirred at 25° C. for 1 hr. Then NaBH₃CN (139.87 mg, 2.23 mmol) was added and the mixture was stirred at 25° C. for 12 hrs. The mixture was filtered and the solid was collected, washed with EtOAc (2×), and dried under vacuum to afford the title compound (100 mg, 30%) as a yellow solid. MS-ESI (m/z) calc'd for C₂₀H₁₉BrN₅O₂ [M+H]⁺: 440.1/442.1. Found 440.2/442.2.

### Step 8: 4-Methoxy-8-((3-(oxazol-5-yl)-1H-indazol-5-yl)amino)-5,6,7,8-tetrahydroquinoline-3-carbonitrile

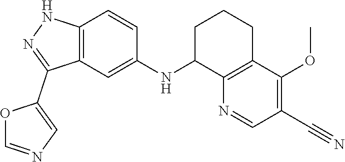 [SMILES: COc1c(C#N)cnc2c1CCCC2Nc1ccc2[nH]nc(-c3cnco3)c2c1]

A mixture of Zn(CN)₂ (64.01 mg, 545.09 umol), 3-bromo-4-methoxy-N-(3-(oxazol-5-yl)-1H-indazol-5-yl)-5,6,7,8-tetrahydroquinolin-8-amine (80 mg, 181.70 umol), Zn (35.64 mg, 545.09 umol), 1,1-bis(diphenylphosphino)ferrocene (10.07 mg, 18.17 umol) and Pd₂dba₃ (16.64 mg, 18.17 umol) in DMA (3 mL) was degassed and purged with N₂ (3×) at 25° C. The mixture was then heated to 100° C. using microwave irradiation under an N₂ atmosphere for 2 hrs. The reaction was filtered and the filtrate was concentrated and purified by preparative-HPLC using Method EY to afford the title compound (36 mg, 51%) as a pink solid. MS-ESI (m/z) calc'd for C₂₁H₁₉N₆O₂ [M+H]⁺: 387.2. Found 387.3.

### Step 9: 4-Methoxy-8-((3-(oxazol-5-yl)-1H-indazol-5-yl)amino)-5,6,7,8-tetrahydroquinoline-3-carbonitrile, enantiomer 1 and 2

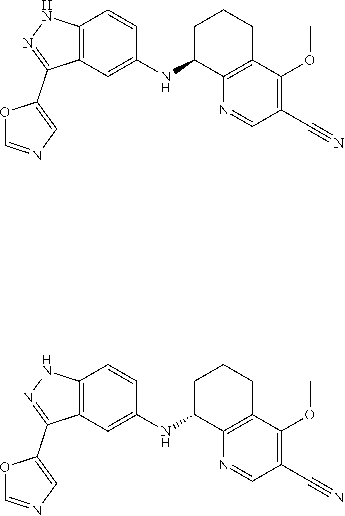 [SMILES: COc1c(C#N)cnc2c1CCC[C@@H]2Nc1ccc2[nH]nc(-c3cnco3)c2c1 ; COc1c(C#N)cnc2c1CCC[C@H]2Nc1ccc2[nH]nc(-c3cnco3)c2c1]

4-Methoxy-8-((3-(oxazol-5-yl)-1H-indazol-5-yl)amino)-5,6,7,8-tetrahydroquinoline-3-carbonitrile (9 mg, 0.02 umol) was subjected to chiral separation using Method EZ to afford 4-methoxy-8-((3-(oxazol-5-yl)-1H-indazol-5-yl)amino)-5,6,7,8-tetrahydroquinoline-3-carbonitrile, enantiomer 1 (2.71 mg, 30%) as a white solid. ¹H NMR (400 MHz, DMSO-d₆) δ 13.09 (s, 1H), 8.69 (s, 1H), 8.48 (s, 1H), 7.66 (s, 1H), 7.36 (d, J=8.88 Hz, 1H), 7.08 (s, 1H), 6.99 (dd, J=8.88, 1.63 Hz, 1H), 5.91 (br d, J=7.38 Hz, 1H), 4.74 (br d, J=6.50 Hz, 1H), 4.24 (s, 3H), 2.61-2.82 (m, 2H), 1.94-2.05 (m, 2H), 1.82-1.93 (m, 2H). MS-ESI (m/z) calc'd for C₂₁H₁₉N₆O₂ [M+H]⁺: 387.2. Found 387.2. A later eluting fraction was also isolated to afford 4-methoxy-8-((3-(oxazol-5-yl)-1H-indazol-5-yl)amino)-5,6,7,8-tetrahydroquinoline-3-carbonitrile, enantiomer 2 (4.32 mg, 46%) as a pale yellow solid. ¹H NMR (400 MHz, DMSO-d₆) δ 13.16 (s, 1H), 8.73 (s, 1H), 8.53 (s, 1H), 7.71 (s, 1H), 7.41 (d, J=9.01 Hz, 1H), 7.13 (s, 1H), 7.04 (dd, J=9.01, 1.88 Hz, 1H), 5.96 (br d, J=6.50 Hz, 1 H), 4.78 (br s, 1H), 4.29 (s, 3H), 2.68-2.87 (m, 2H), 1.98-2.05 (m, 2H), 1.86-1.97 (m, 2H). MS-ESI (m/z) calc'd for C₂₁H₁₉N₆O₂ [M+H]⁺: 387.2. Found 387.1.

### Example 147: 4-(Difluoromethyl)-8-((3-(oxazol-5-yl)-1H-indazol-5-yl)amino)-5,6,7,8-tetrahydroquinoline-3-carbonitrile, enantiomer 1 and 2

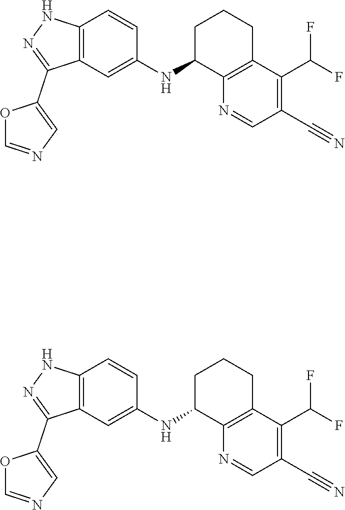 [SMILES: N#Cc1cnc2c(c1C(F)F)CCC[C@@H]2Nc1ccc2[nH]nc(-c3cnco3)c2c1 ; N#Cc1cnc2c(c1C(F)F)CCC[C@H]2Nc1ccc2[nH]nc(-c3cnco3)c2c1]

### Step 1: 2-(2,2-Difluoroacetyl)cyclohexanone

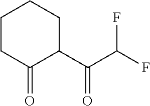 [SMILES: O=C1CCCCC1C(=O)C(F)F]

To a solution of cyclohexanone (2 g, 20.38 mmol) in THF (20 mL) was added LDA (2 M, 10.19 mL) slowly at −70° C. under an N₂ atmosphere. The mixture was stirred at −70° C. for 5 minutes; then methyl 2,2-difluoroacetate (2.24 g, 20.38 mmol) was added slowly at −70° C. The resulting mixture was then stirred at 20° C. for 2 hrs under an N₂ atmosphere. The reaction mixture was adjusted to pH=3 with 1 M HCl at −70° C. and then extracted with EtOAc (3×). The combined organic layers were dried over Na₂SO₄, filtered, and evaporated to afford the title compound (3.2 g, 89%) as yellow oil. MS-ESI (m/z) calc'd for C₈H₉F₂₀₂ [M−H]⁻: 175.1. Found 175.0.

### Step 2: 4-(Difluoromethyl)-2-hydroxy-5,6,7,8-tetrahydroquinoline-3-carbonitrile

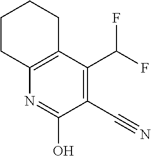 [SMILES: N#Cc1c(O)nc2c(c1C(F)F)CCCC2]

To a solution of 2-(2,2-difluoroacetyl)cyclohexanone (1.00 g, 5.68 mmol) and 2-cyanoacetamide (620.46 mg, 7.38 mmol) in 2-propanol (3 mL) was added KF (65.96 mg, 1.14 mmol) at 20° C. The mixture was then stirred at 80° C. for 12 hrs. A solid formed that was collected by filtration, washed with MeOH (2×), and dried under vacuum to afford the title compound (440 mg, 34%) as a gray solid. ¹H NMR (400 MHz, DMSO-d₆) δ 6.95-7.25 (m, 1H) 2.63 (br s, 2H) 2.55 (br s, 2H) 1.69 (br t, J=3 Hz, 4H). MS-ESI (m/z) calc'd for C₁₁H₁₁F₂N₂O [M+H]⁺: 225.1. Found 225.2.

### Step 3: 2-Chloro-4-(difluoromethyl)-5,6,7,8-tetrahydroquinoline-3-carbonitrile

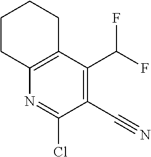 [SMILES: N#Cc1c(Cl)nc2c(c1C(F)F)CCCC2]

A solution of 4-(difluoromethyl)-2-hydroxy-5,6,7,8-tetrahydroquinoline-3-carbonitrile (440 mg, 1.96 mmol) in POCl₃ (3.01 g, 19.62 mmol) was stirred at 100° C. for 12 hrs. The reaction mixture was poured into saturated aqueous NaHCO₃ and extracted with EtOAc (3×). The combined organic layers were dried over Na₂SO₄, filtered, and concentrated to give a residue. The residue was purified by flash silica gel chromatography (ISCO; 4 g SepaFlash) using a 0-20% EtOAc/petroleum ether gradient elute to afford the title compound (340 mg, 69%) as a colorless oil. ¹H NMR (400 MHz, CDCl₃) δ 6.79-7.09 (m, 1H) 2.94-3.04 (m, 4H) 1.80-1.94 (m, 4H). MS-ESI (m/z) calc'd for C₁₁H₁₀ClF₂N₂ [M+H]⁺: 243.0/245.0. Found 243.0/245.1.

### Step 4: 4-(Difluoromethyl)-5,6,7,8-tetrahydroquinoline-3-carbonitrile

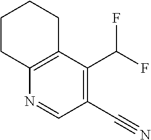 [SMILES: N#Cc1cnc2c(c1C(F)F)CCCC2]

To a solution of 2-chloro-4-(difluoromethyl)-5,6,7,8-tetrahydroquinoline-3-carbonitrile (0.34 g, 1.40 mmol) in MeOH (5 mL) was added wet 10% Pd/C (0.3 g) and ammonium formate (265.08 mg, 4.20 mmol). The mixture was then stirred at 60° C. for 2 hrs and filtered. The filtrate was evaporated to afford the title compound (235 mg, 80%) as a yellow solid. ¹H NMR (400 MHz, CDCl₃) δ 8.66 (s, 1H) 6.74-7.03 (m, 1H) 2.97 (dt, J=20, 6 Hz, 4H) 1.76-1.90 (m, 4H). MS-ESI (m/z) calc'd for C₁₁H₁₁F₂N₂ [M+H]⁺: 209.1. Found 209.0.

### Step 5: 3-Cyano-4-(difluoromethyl)-5,6,7,8-tetrahydroquinoline 1-oxide

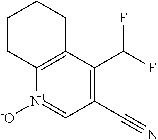

To a solution of 4-(difluoromethyl)-5,6,7,8-tetrahydroquinoline-3-carbonitrile (270 mg, 1.30 mmol) in DCM (4 mL) was added m-CPBA (419.59 mg, 1.95 mmol). The mixture was stirred at 40° C. for 2 hrs. The reaction mixture was quenched with saturated Na₂SO₃ and extracted with EtOAc (3×), the combined organic layer was dried over Na₂SO₄, filtered and concentrated to afford the title compound (230 mg, 79%) as a yellow gum. MS-ESI (m/z) calc'd for C₁₁H₁₁F₂N₂O [M+H]⁺: 225.1. Found 225.1.

### Step 6: 4-(Difluoromethyl)-8-hydroxy-5,6,7,8-tetrahydroquinoline-3-carbonitrile

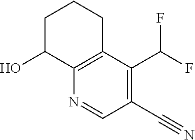 [SMILES: N#Cc1cnc2c(c1C(F)F)CCCC2O]

A solution of 3-cyano-4-(difluoromethyl)-5,6,7,8-tetrahydroquinoline 1-oxide (192 mg, 856.35 umol) in TFAA (1.26 g, 5.99 mmol) was stirred at 50° C. for 2 hrs. The reaction mixture was concentrated to give a residue that was diluted with 2 N aqueous NaOH and extracted with EtOAc (4×). The combined organic layers were dried over Na₂SO₄, filtered, and concentrated to give a residue. The residue was purified by preparative-TLC (SiO₂, petroleum ether/EtOAc=1/1, Rf=0.37) to afford the title compound (80 mg, 41%) as a yellow gum. ¹H NMR (400 MHz, CDCl₃) δ 8.74 (s, 1H) 6.75-7.05 (m, 1H) 4.65-4.71 (m, 1H) 3.80 (br s, 1H) 2.89-3.09 (m, 2H) 2.24-2.32 (m, 1H) 1.96-2.07 (m, 1H) 1.70-1.85 (m, 2H). MS-ESI (m/z) calc'd for C₁₁H₁₁F₂N₂O [M+H]⁺: 225.1. Found 225.1.

### Step 7: 4-(Difluoromethyl)-8-oxo-5,6,7,8-tetrahydroquinoline-3-carbonitrile

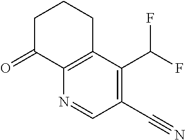 [SMILES: N#Cc1cnc2c(c1C(F)F)CCCC2=O]

To a solution of 4-(difluoromethyl)-8-hydroxy-5,6,7,8-tetrahydroquinoline-3-carbonitrile (80 mg, 356.81 umol) in DCM (6 mL) was added Dess-Martin periodinane (181.61 mg, 428.18 umol). The mixture was stirred at 20° C. for 12 hrs. The reaction mixture was quenched with saturated aqueous NaHCO₃ and extracted with EtOAc (4×). The combined organic layers were dried over Na₂SO₄, filtered, and concentrated to give a residue. The residue was purified by preparative-TLC (SiO₂, petroleum ether/EtOAc=1/3, Rf=0.43) to afford the title compound (67 mg, 84%) as a red solid. MS-ESI (m/z) calc'd for C₁₁H₉F₂N₂O [M+H]⁺: 223.1. Found 223.0.

### Step 8: rac-4-(Difluoromethyl)-8-((3-(oxazol-5-yl)-1H-indazol-5-yl)amino)-5,6,7,8-tetrahydroquinoline-3-carbonitrile

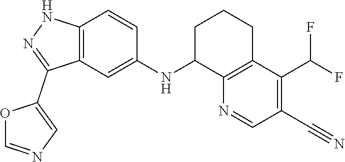 [SMILES: N#Cc1cnc2c(c1C(F)F)CCCC2Nc1ccc2[nH]nc(-c3cnco3)c2c1]

To a solution of 4-(difluoromethyl)-8-oxo-5,6,7,8-tetrahydroquinoline-3-carbonitrile (67 mg, 301.54 umol) and 3-oxazol-5-yl-1H-indazol-5-amine (60.37 mg, 301.54 umol) in MeOH (4 mL) was added AcOH (1.81 mg, 30.15 umol) to pH=5. The mixture was stirred at 20° C. for 1 hr, then NaBH₃CN (94.75 mg, 1.51 mmol) was added and the mixture was stirred at 20° C. for 1.5 hr. The reaction mixture was concentrated and purified by preparative-HPLC using Method FA to afford the title compound (18 mg, 14%) as a yellow solid. MS-ESI (m/z) calc'd for C₂₁H₁₇F₂N₆O [M+H]⁺: 407.1. Found 407.0.

### Step 9: 4-(Difluoromethyl)-8-((3-(oxazol-5-yl)-H-indazol-5-yl)amino)-5,6,7,8-tetrahydroquinoline-3-carbonitrile, enantiomer 1 and 2

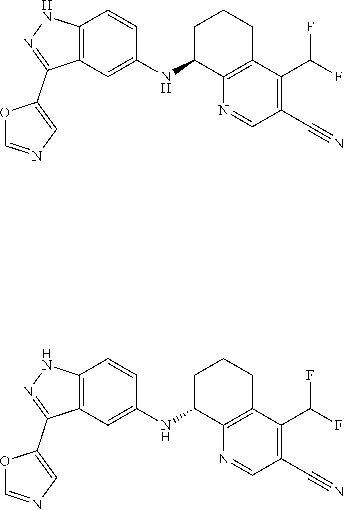 [SMILES: N#Cc1cnc2c(c1C(F)F)CCC[C@@H]2Nc1ccc2[nH]nc(-c3cnco3)c2c1 ; N#Cc1cnc2c(c1C(F)F)CCC[C@H]2Nc1ccc2[nH]nc(-c3cnco3)c2c1]

4-(Difluoromethyl)-8-((3-(oxazol-5-yl)-1H-indazol-5-yl)amino)-5,6,7,8-tetrahydroquinoline-3-carbonitrile (9 mg) was subjected to chiral separation using Method FB to afford the two enantiomers. The first eluting fraction was then re-purified by prep-HPLC using Method FC to afford 4-(difluoromethyl)-8-((3-(oxazol-5-yl)-1H-indazol-5-yl)amino)-5,6,7,8-tetrahydroquinoline-3-carbonitrile, enantiomer 1 (1.84 mg, 20%) as a yellow solid. ¹H NMR (400 MHz, DMSO-d₆) δ 13.09 (s, 1H) 9.00 (s, 1H) 8.47 (s, 1H) 7.66 (s, 1H) 7.32-7.62 (m, 2H) 7.10 (s, 1H) 6.98 (dd, J=9, 2 Hz, 1H) 6.03 (d, J=8 Hz, 1H) 4.83-4.91 (m, 1H) 3.01-3.12 (m, 1H) 2.88-2.99 (m, 1H) 1.83-2.07 (m, 4H). MS-ESI (m/z) calc'd for C₂₁H₁₇F₂N₆O [M+H]⁺: 407.1. Found 407.2. The later eluting fraction was also re-purified by prep-HPLC using Method FC to afford 4-(difluoromethyl)-8-((3-(oxazol-5-yl)-1H-indazol-5-yl)amino)-5,6,7,8-tetrahydroquinoline-3-carbonitrile, enantiomer 2 (1.68 mg, 18%). ¹H NMR (400 MHz, DMSO-d₆) δ 13.10 (br s, 1H) 9.00 (s, 1H) 8.47 (s, 1H) 7.66 (s, 1H) 7.32-7.62 (m, 2H) 7.09 (s, 1H) 6.98 (dd, J=9, 2 Hz, 1H) 6.03 (d, J=8 Hz, 1H) 4.83-4.91 (m, 1H) 3.02-3.12 (m, 1H) 2.88-3.00 (m, 1H) 1.83-2.08 (m, 4H). MS-ESI (m/z) calc'd for C₂₁H₁₇F₂N₆O [M+H]⁺: 407.1. Found 407.2.

### Example 148: 8-((3-(1H-Pyrrol-2-yl)-1H-indazol-5-yl)amino)-5,6,7,8-tetrahydroquinoline-3-carbonitrile, enantiomer 1 and 2

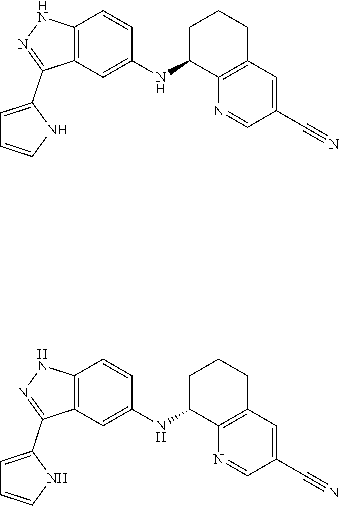 [SMILES: N#Cc1cnc2c(c1)CCC[C@@H]2Nc1ccc2[nH]nc(-c3ccc[nH]3)c2c1 ; N#Cc1cnc2c(c1)CCC[C@H]2Nc1ccc2[nH]nc(-c3ccc[nH]3)c2c1]

### Step 1: tert-Butyl 2-(5-amino-1H-indazol-3-yl)-1H-pyrrole-1-carboxylate

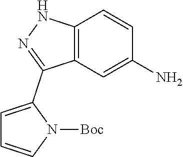 [SMILES: CC(C)(C)OC(=O)n1cccc1-c1n[nH]c2ccc(N)cc12]

A mixture of 3-iodo-1H-indazol-5-amine (800 mg, 3.09 mmol), tert-butyl 2-(4,4,5,5-tetramethyl-1,3,2-dioxaborolan-2-yl)-1H-pyrrole-1-carboxylate (995.90 mg, 3.40 mmol), Pd(Amphos)Cl₂ (218.67 mg, 308.83 umol), AcOK (909.24 mg, 9.26 mmol) in EtOH (15 mL) and H₂O (3 mL) was degassed and purged with N₂ (3×) at 20° C., and then the mixture was stirred at 80° C. for 2 hrs under an N₂ atmosphere. The reaction mixture was concentrated to give a residue that was diluted with H₂O and extracted with EtOAc (3×). The combined organic layers were washed with brine (2×), dried over Na₂SO₄, filtered, and concentrated. The residue was purified by flash silica gel chromatography (ISCO; 20 g SepaFlash column) using a 0-40% EtOAc/petroleum ether gradient eluent to afford the title compound (500 mg, 54%) as a yellow solid. MS-ESI (m/z) calc'd for C₁₆H₁₉N₄O₂ [M−tBu+H]⁺: 299.1. Found 243.2.

### Step 2: 3-(1H-Pyrrol-2-yl)-1H-indazol-5-amine

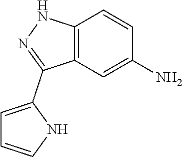 [SMILES: Nc1ccc2[nH]nc(-c3ccc[nH]3)c2c1]

A mixture of tert-butyl 2-(5-amino-1H-indazol-3-yl)-1H-pyrrole-1-carboxylate (350 mg, 1.17 mmol) in HCl/EtOAc (4 M, 5 mL) was stirred at 20° C. for 2 hrs. The reaction was combined with another 150 mg scale reaction before work up. The final mixture was concentrated under reduced pressure to remove solvent. The residue was diluted with saturated aqueous Na₂CO₃ to pH=8 and extracted with EtOAc (3×). The combined organic phases were dried over anhydrous Na₂SO₄, filtered, and the filtrate was concentrated to give a residue. The residue was purified by flash silica gel chromatography (ISCO; 4 g SepaFlash column) using a 0-36% EtOAc/petroleum ether gradient eluent to afford the title compound (190 mg, 57%) as a brown solid. MS-ESI (m/z) calc'd for C₁₁H₁₁N₄ [M+H]⁺: 199.1. Found 199.3.

### Step 3: 8-((3-(1H-Pyrrol-2-yl)-1H-indazol-5-yl)amino)-5,6,7,8-tetrahydroquinoline-3-carbonitrile

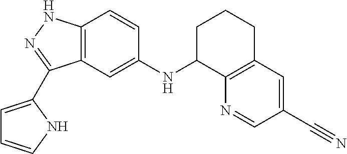 [SMILES: N#Cc1cnc2c(c1)CCCC2Nc1ccc2[nH]nc(-c3ccc[nH]3)c2c1]

To a solution of 3-(1H-pyrrol-2-yl)-1H-indazol-5-amine (180 mg, 908.07 umol) in MeOH (3 mL) were added AcOH (5.45 mg, 90.81 umol) and 8-oxo-5,6,7,8-tetrahydroquinoline-3-carbonitrile (156.35 mg, 908.07 umol). The mixture was stirred at 20° C. for 1 hr; then NaBH₃CN (342.38 mg, 5.45 mmol) was added and the mixture was stirred at 20° C. for an additional 2 hrs. The reaction mixture was concentrated and purified by preparative-HPLC using Method FD to afford the title compound (25 mg, 8%) as a white solid. MS-ESI (m/z) calc'd for C₂₁H₁₉N₆ [M+H]⁺: 355.2. Found 355.3.

### Step 4: 8-((3-(1H-Pyrrol-2-yl)-1H-indazol-5-yl)amino)-5,6,7,8-tetrahydroquinoline-3-carbonitrile, enantiomer 1 and 2

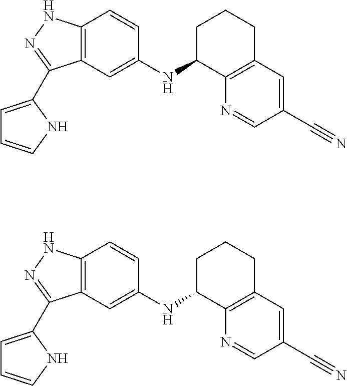 [SMILES: N#Cc1cnc2c(c1)CCC[C@@H]2Nc1ccc2[nH]nc(-c3ccc[nH]3)c2c1 ; N#Cc1cnc2c(c1)CCC[C@H]2Nc1ccc2[nH]nc(-c3ccc[nH]3)c2c1]

8-((3-(1H-Pyrrol-2-yl)-1H-indazol-5-yl)amino)-5,6,7,8-tetrahydroquinoline-3-carbonitrile (25 mg) was subjected to chiral separation using Method FE to afford 8-((3-(1H-pyrrol-2-yl)-1H-indazol-5-yl)amino)-5,6,7,8-tetrahydroquinoline-3-carbonitrile, enantiomer 1 (9.45 mg, 38%) as a white solid. ¹H NMR (400 MHz, DMSO-d₆) δ 12.48 (s, 1H), 11.14 (br s, 1H), 8.76 (d, J=1.8 Hz, 1H), 8.08 (d, J=1.6 Hz, 1H), 7.22 (d, J=8.9 Hz, 1H), 7.04 (s, 1H), 6.89 (dd, J=1.8, 8.9 Hz, 1H), 6.73 (br d, J=1.1 Hz, 1H), 6.50 (br s, 1H), 6.09 (q, J=2.6 Hz, 1H), 5.71 (d, J=7.1 Hz, 1H), 4.72-4.64 (m, 1H), 2.90-2.71 (m, 2H), 2.04-1.93 (m, 2H), 1.91-1.72 (m, 2H). MS-ESI (m/z) calc'd for C₂₁H₁₉N₆ [M+H]⁺: 355.2. Found 355.2. A later eluting fraction was also isolated to afford 8-((3-(1H-pyrrol-2-yl)-1H-indazol-5-yl)amino)-5,6,7,8-tetrahydroquinoline-3-carbonitrile, enantiomer 2 (6.44 mg, 26%) as a white solid. ¹H NMR (400 MHz, DMSO-d₆) δ 12.48 (br s, 1H), 11.14 (br s, 1H), 8.75 (d, J=1.8 Hz, 1H), 8.08 (d, J=1.6 Hz, 1H), 7.22 (d, J=8.9 Hz, 1H), 7.04 (s, 1H), 6.89 (dd, J=1.7, 8.9 Hz, 1H), 6.73 (br d, J=1.1 Hz, 1H), 6.50 (br s, 1H), 6.09 (q, J=2.5 Hz, 1H), 5.71 (d, J=7.3 Hz, 1H), 4.72-4.58 (m, 1H), 2.88-2.69 (m, 2H), 2.02-1.92 (m, 2H), 1.92-1.73 (m, 2H) MS-ESI (m/z) calc'd for C₂₁H₁₉N₆ [M+H]⁺: 355.2. Found 355.2.

### Example 149: 8-((3-(1-Methyl-1H-pyrrol-3-yl)-1H-indazol-5-yl)amino)-5,6,7,8-tetrahydroquinoline-3-carbonitrile, enantiomer 1 and 2

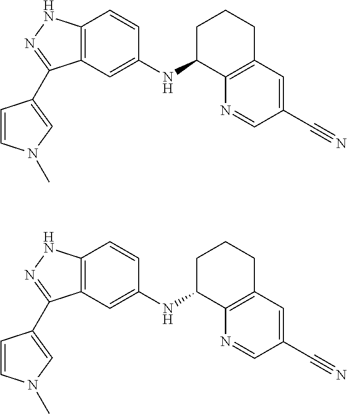 [SMILES: Cn1ccc(-c2n[nH]c3ccc(N[C@H]4CCCc5cc(C#N)cnc54)cc23)c1 ; Cn1ccc(-c2n[nH]c3ccc(N[C@@H]4CCCc5cc(C#N)cnc54)cc23)c1]

### Step 1: 3-(1-Methyl-1H-pyrrol-3-yl)-1H-indazol-5-amine

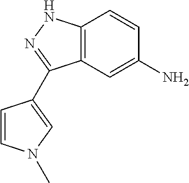 [SMILES: Cn1ccc(-c2n[nH]c3ccc(N)cc23)c1]

To a solution of 3-iodo-1H-indazol-5-amine (210 mg, 656.64 umol) in EtOH (3 mL) and H₂O (1.5 mL) were added AcOK (128.89 mg, 1.31 mmol) and 1-methyl-3-(4,4,5,5-tetramethyl-1,3,2-dioxaborolan-2-yl)pyrrole (135.97 mg, 656.64 umol), and Pd(Amphos)Cl₂ (46.49 mg, 65.66 umol) at 20° C. The mixture was stirred at 100° C. for 2 hrs under an N₂ atmosphere. The reaction mixture was filtered and the filtrate was concentrated to give a residue. The residue was purified by flash silica gel chromatography (ISCO; 12 g SepaFlash column) using a 0-48% EtOAc/petroleum ether gradient eluent to afford the title compound (67.95 mg, 48%) as a brown gum. MS-ESI (m/z) calc'd for C₁₂H₁₃N₄ [M+H]⁺: 213.1. Found 213.2.

### Step 2: 8-((3-(1-Methyl-1H-pyrrol-3-yl)-1H-indazol-5-yl)amino)-5,6,7,8-tetrahydroquinoline-3-carbonitrile

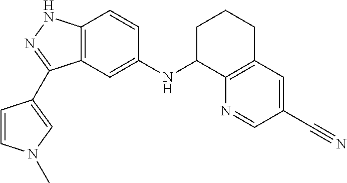 [SMILES: Cn1ccc(-c2n[nH]c3ccc(NC4CCCc5cc(C#N)cnc54)cc23)c1]

To a solution of 3-(1-methyl-1H-pyrrol-3-yl)-1H-indazol-5-amine (70 mg, 329.80 umol) and 8-oxo-5,6,7,8-tetrahydroquinoline-3-carbonitrile (56.79 mg, 329.80 umol) in MeOH (1.5 mL) was added AcOH (1.98 mg, 32.98 umol) to adjust pH=5, the mixture was stirred at 20° C. for 2 hrs. Then NaBH₃CN (62.18 mg, 989.40 umol) was added, the mixture was stirred at 20° C. for 2 hrs. The reaction mixture was concentrated and purified by preparative-HPLC using Method FF to afford the title compound (27 mg, 16%) as a pale yellow solid TFA salt. MS-ESI (m/z) calc'd for C₂₂H₂₁N₆ [M+H]⁺: 369.2. Found 369.3.

### Step 3: 8-((3-(1-Methyl-1H-pyrrol-3-yl)-1H-indazol-5-yl)amino)-5,6,7,8-tetrahydroquinoline-3-carbonitrile, enantiomer 1 and 2

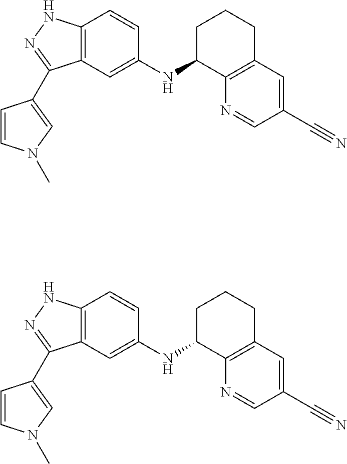 [SMILES: Cn1ccc(-c2n[nH]c3ccc(N[C@H]4CCCc5cc(C#N)cnc54)cc23)c1 ; Cn1ccc(-c2n[nH]c3ccc(N[C@@H]4CCCc5cc(C#N)cnc54)cc23)c1]

8-((3-(1-Methyl-1H-pyrrol-3-yl)-1H-indazol-5-yl)amino)-5,6,7,8-tetrahydroquinoline-3-carbonitrile (27 mg) was subjected to chiral separation using Method FG to afford 8-((3-(1-methyl-1H-pyrrol-3-yl)-1H-indazol-5-yl)amino)-5,6,7,8-tetrahydroquinoline-3-carbonitrile, enantiomer 1 (6.52 mg, 24%) as a yellow gum. ¹H NMR (400 MHz, DMSO-d₆) δ 12.27 (s, 1H), 8.82 (d, J=1.98 Hz, 1H), 8.14 (s, 1H), 7.19-7.27 (m, 2H), 7.07 (s, 1H), 6.90 (br d, J=8.82 Hz, 1H), 6.77 (s, 1H), 6.48 (s, 1H), 5.68 (br d, J=7.50 Hz, 1H), 4.73 (br d, J=7.06 Hz, 1H), 3.69 (s, 3H), 2.76-2.95 (m, 2H), 1.77-2.05 (m, 4H). MS-ESI (m/z) calc'd for C₂₂H₂₁N₆ [M+H]⁺: 369.2. Found 369.2. A later eluting fraction was also isolated to afford 8-((3-(1-methyl-1H-pyrrol-3-yl)-1H-indazol-5-yl)amino)-5,6,7,8-tetrahydroquinoline-3-carbonitrile, enantiomer 2 (5.85 mg, 21%) as a pale yellow solid. ¹H NMR (400 MHz, DMSO-d₆) δ 12.27 (br s, 1H), 8.82 (d, J=1.98 Hz, 1H), 8.14 (d, J=1.76 Hz, 1H), 7.20-7.28 (m, 2H), 7.06 (s, 1H), 6.90 (dd, J=1.98, 8.82 Hz, 1H), 6.77 (t, J=2.32 Hz, 1H), 6.46-6.51 (m, 1H), 5.68 (d, J=7.28 Hz, 1H), 4.73 (br d, J=6.84 Hz, 1H), 3.69 (s, 3H), 2.75-2.95 (m, 2H), 1.77-2.07 (m, 4H). MS-ESI (m/z) calc'd for C₂₂H₂₁N₆ [M+H]⁺: 369.2. Found 369.3.

### Example 150: 4-Methyl-8-((3-(oxazol-5-yl)-1H-indazol-5-yl)amino)-5,6,7,8-tetrahydroquinoline-3-carbonitrile, enantiomer 1 and 2

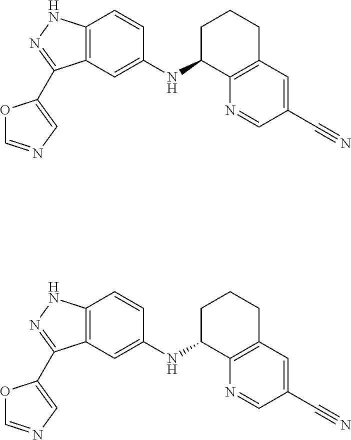 [SMILES: N#Cc1cnc2c(c1)CCC[C@@H]2Nc1ccc2[nH]nc(-c3cnco3)c2c1 ; N#Cc1cnc2c(c1)CCC[C@H]2Nc1ccc2[nH]nc(-c3cnco3)c2c1]

### Step 1: 2-Amino-4-methyl-5,6,7,8-tetrahydroquinoline-3-carbonitrile

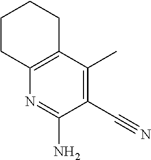

To a solution of cyclohexanone (70 g, 713.25 mmol), acetaldehyde (78.55 g, 713.25 mmol) and malononitrile (47.12 g, 713.25 mmol) in EtOH (1000 mL) was added NH₄OAc (82.47 g, 1.07 mol) at 20° C. The mixture was stirred at 80° C. for 1 hr. Then the mixture was stirred at 20° C. for an additional 3 hrs. A solid formed that was collected by filtration and dried to afford the title compound (4.6 g, 3%) as a yellow solid. ¹H NMR (400 MHz, DMSO-d₆) δ 6.37 (s, 2H), 2.61 (br s, 2H), 2.46 (br s, 2H), 2.22 (s, 3H), 1.71 (br t, J=2.8 Hz, 4H). MS-ESI (m/z) calc'd for C₁₁H₁₄N₃ [M+H]⁺: 188.1. Found 188.1.

### Step 2: 2-Hydroxy-4-methyl-5,6,7,8-tetrahydroquinoline-3-carbonitrile and 2-chloro-4-methyl-5,6,7,8-tetrahydroquinoline-3-carbonitrile

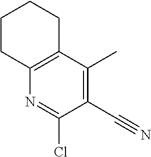

To a solution of 2-amino-4-methyl-5,6,7,8-tetrahydroquinoline-3-carbonitrile (1.7 g, 9.08 mmol) in DMF (100 mL) was added CuCl₂ (7.32 g, 54.48 mmol) and isopentyl nitrite (6.38 g, 54.48 mmol) at 20° C. The mixture was stirred at 40° C. for 12 hrs. This procedure was conducted three times and the mixtures were combined and concentrated. The residue was diluted with H₂O and filtered. The filtrate was extracted with EtOAc (3×). The combined organic phases were passed through a phase separator and evaporated to dryness to obtain material that was further purified by flash silica gel column chromatography (ISCO; 40 g SepaFlash column) using a 0-5% EtOAc/petroleum ether gradient eluent to afford the title compound (2.6 g, 46%) as a white solid. MS-ESI (m/z) calc'd for C₁₁H₁₂ClN₂ [M+H]⁺: 207.1/209.1. Found 207.2/209.2.

### Step 3: 4-Methyl-5,6,7,8-tetrahydroquinoline-3-carbonitrile

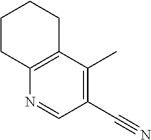

A solution of 2-chloro-4-methyl-5,6,7,8-tetrahydroquinoline-3-carbonitrile (4.1 g, 19.84 mmol), Zn (4.06 g, 62.09 mmol) and NaOAc (1.63 g, 19.84 mmol) in AcOH (20.25 g, 337.25 mmol) was stirred at 70° C. for 2 hrs. H₂O (10 mL) was then added to the mixture at 70° C. and the mixture was stirred at 70° C. for 12 hrs. The mixture was filtered, the filtrate was diluted with H₂O and extracted with EtOAc (5×). The combined organic phases were dried over anhydrous Na₂SO₄, filtered, and the filtrate was evaporated to afford the title compound (3.4 g, 99%) as a yellow oil. MS-ESI (m/z) calc'd for C₁₁H₁₃N₂ [M+H]⁺: 173.1. Found 173.0.

### Step 4: 3-Cyano-4-methyl-5,6,7,8-tetrahydroquinoline 1-oxide

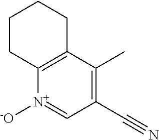

To a solution of 4-methyl-5,6,7,8-tetrahydroquinoline-3-carbonitrile (3.4 g, 19.74 mmol) in DCM (40 mL) was added m-CPBA (6.01 g, 29.61 mmol). The mixture was stirred at 20° C. for 12 hrs. The reaction mixture was cooled to 0° C. and quenched by 10% Na₂SO₃ aqueous solution and filtered. Then the filtrate was extracted with DCM (3×). The combined organic phases were dried with anhydrous Na₂SO₄, filtered, and the filtrate was evaporated to afford the title compound (2.5 g, 67%) as a yellow solid. MS-ESI (m/z) calc'd for C₁₁H₁₃N₂₀ [M+H]⁺: 189.1. Found 189.0.

### Step 5: 8-Hydroxy-4-methyl-5,6,7,8-tetrahydroquinoline-3-carbonitrile

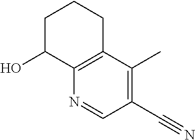

A solution of 3-cyano-4-methyl-5,6,7,8-tetrahydroquinoline 1-oxide (5.1 g, 27.10 mmol) in TFAA (50 mL) was stirred at 50° C. for 12 hrs. The reaction mixture was concentrated to give a residue. The residue was basified with 2 N NaOH aqueous solution to pH=9 and the mixture was stirred at 20° C. for 0.5 hr. The mixture was then extracted with EtOAc (3×). The combined organic phases were dried over anhydrous Na₂SO₄, filtered, and the filtrate was evaporated. The residue was purified by flash silica gel column chromatography (ISCO; 12 g SepaFlash column) using a 0-12% EtOAc/petroleum ether gradient eluent to afford the title compound (1.9 g, 37%) as a red solid. ¹H NMR (400 MHz, CDCl₃) δ 8.62 (s, 1H), 4.75-4.67 (m, 1H), 4.42-3.91 (m, 1H), 2.80-2.68 (m, 2H), 2.46 (s, 3H), 2.33-2.24 (m, 1H), 2.16-2.06 (m, 1H), 1.91-1.75 (m, 2H). MS-ESI (m/z) calc'd for C₁₁H₁₃N₂O [M+H]⁺: 189.1. Found 189.0.

### Step 6: 4-Methyl-8-oxo-5,6,7,8-tetrahydroquinoline-3-carbonitrile

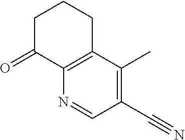

To a solution of 8-hydroxy-4-methyl-5,6,7,8-tetrahydroquinoline-3-carbonitrile (1.9 g, 10.09 mmol) in DCM (40 mL) was added Dess-Martin periodinane (5.14 g, 12.11 mmol). The mixture was stirred at 20° C. for 12 hrs. The reaction mixture was quenched with saturated aqueous Na₂CO₃ at 0° C., then the mixture was extracted with DCM (3×). The combined organic phase was washed with saturated aqueous Na₂CO₃ (6×), dried over anhydrous Na₂SO₄, filtered, and the filtrate was evaporated. The residue was purified by flash silica gel column chromatography (ISCO; 20 g SepaFlash column) using a 0-35% EtOAc/petroleum ether gradient eluent to afford the title compound (1.18 g, 63%) as a pale yellow solid. ¹H NMR (400 MHz, CDCl₃) δ 8.85 (s, 1H), 3.01 (t, J=6.1 Hz, 2H), 2.87-2.81 (m, 2H), 2.60 (s, 3H), 2.30-2.22 (m, 2H). MS-ESI (m/z) calc'd for C₁₁H₁₁N₂O [M+H]⁺: 187.1. Found 187.0.

### Step 7: 4-Methyl-8-((3-(oxazol-5-yl)-1H-indazol-5-yl)amino)-5,6,7,8-tetrahydroquinoline-3-carbonitrile

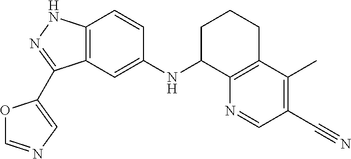 [SMILES: Cc1c(C#N)cnc2c1CCCC2Nc1ccc2[nH]nc(-c3cnco3)c2c1]

To a solution of 4-methyl-8-oxo-5,6,7,8-tetrahydroquinoline-3-carbonitrile (150 mg, 805.54 umol) and 3-(oxazol-5-yl)-1H-indazol-5-amine (161.27 mg, 805.54 umol) in MeOH (15 mL) was added AcOH (4.84 mg, 80.55 umol) to pH=5 at 20° C. The mixture was stirred at 20° C. for 1 hr. Then NaBH₃CN (303.73 mg, 4.83 mmol) was added to the mixture at 20° C. The mixture was stirred at 20° C. for 12 hrs. A solid was collected by filtration and dried under vacuum to afford the title compound (137 mg, 46%) as a yellow solid. MS-ESI (m/z) calc'd for C₂₁H₁₉N₆O [M+H]⁺: 371.2. Found 371.1.

### Step 8: 4-Methyl-8-((3-(oxazol-5-yl)-1H-indazol-5-yl)amino)-5,6,7,8-tetrahydroquinoline-3-carbonitrile, enantiomer 1 and 2

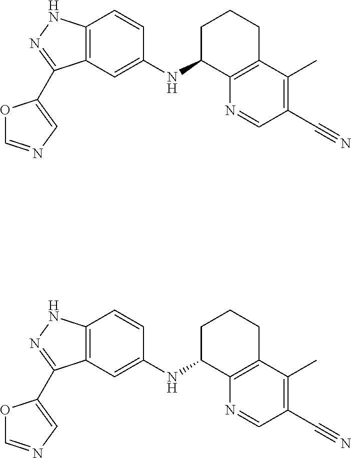 [SMILES: Cc1c(C#N)cnc2c1CCC[C@@H]2Nc1ccc2[nH]nc(-c3cnco3)c2c1 ; Cc1c(C#N)cnc2c1CCC[C@H]2Nc1ccc2[nH]nc(-c3cnco3)c2c1]

4-Methyl-8-((3-(oxazol-5-yl)-1H-indazol-5-yl)amino)-5,6,7,8-tetrahydroquinoline-3-carbonitrile (9 mg) was subjected to chiral separation using Method FH to afford the two enantiomers. The first eluting fraction was further purified by preparative-HPLC using Method FI to afford 4-methyl-8-((3-(oxazol-5-yl)-1H-indazol-5-yl)amino)-5,6,7,8-tetrahydroquinoline-3-carbonitrile, enantiomer 1 (4.05 mg, 45%) as a gray solid. ¹H NMR (400 MHz, DMSO-d₆) δ 13.09 (br s, 1H), 8.74 (s, 1H), 8.48 (s, 1H), 7.65 (s, 1H), 7.35 (d, J=8.8 Hz, 1H), 7.08 (s, 1H), 7.00 (br d, J=8.8 Hz, 1H), 5.94 (br d, J=7.5 Hz, 1H), 4.75 (br s, 1H), 2.86-2.66 (m, 2H), 2.44 (s, 3H), 2.01-1.85 (m, 4H). MS-ESI (m/z) calc'd for C₂₁H₁₉N₆O [M+H]⁺: 371.2. Found 371.2. A later eluting fraction was also isolated to afford 4-methyl-8-((3-(oxazol-5-yl)-1H-indazol-5-yl)amino)-5,6,7,8-tetrahydroquinoline-3-carbonitrile, enantiomer 2 (4.13 mg, 45%) as a yellow solid. ¹H NMR (400 MHz, DMSO-d₆) δ 13.10 (s, 1H), 8.74 (s, 1H), 8.48 (s, 1H), 7.66 (s, 1H), 7.36 (d, J=8.9 Hz, 1H), 7.14-6.94 (m, 2H), 5.98 (br s, 1H), 4.76 (br s, 1H), 2.89-2.62 (m, 2H), 2.45 (s, 3H), 2.04-1.80 (m, 4H). MS-ESI (m/z) calc'd for C₂₁H₁₉N₆O [M+H]⁺: 371.2. Found 371.2.

### Example 151: 2,2,4-Trifluoro-1-((3-(oxazol-5-yl)-1H-indazol-5-yl)amino)-2,3-dihydro-1H-indene-5-carbonitrile, enantiomer 1 and 2

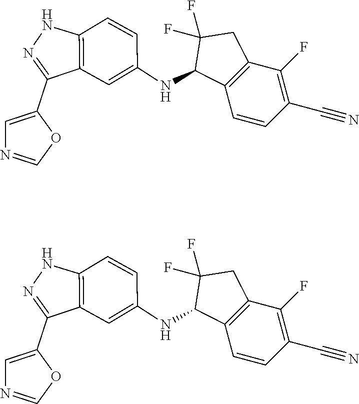 [SMILES: N#Cc1ccc2c(c1F)CC(F)(F)[C@@H]2Nc1ccc2[nH]nc(-c3cnco3)c2c1 ; N#Cc1ccc2c(c1F)CC(F)(F)[C@H]2Nc1ccc2[nH]nc(-c3cnco3)c2c1]

### Step 1: 5-Bromo-2,2,4-trifluoro-2,3-dihydro-1H-inden-1-one

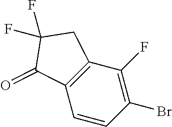 [SMILES: O=C1c2ccc(Br)c(F)c2CC1(F)F]

To a solution of 5-bromo-4-fluoro-2,3-dihydro-1H-inden-1-one (200 mg, 873.19 umol) in THF (10 mL) was added NaH (111.76 mg, 2.79 mmol) at 0° C. The mixture was stirred at 0° C. for 10 minutes. Then 1-chloromethyl-4-fluoro-1,4-diazoniabicyclo[2.2.2]octane bis(tetrafluoroborate) (649.60 mg, 1.83 mmol) was added to the mixture. The mixture was stirred at 20° C. for 1 hr. This procedure was conducted four times and the mixtures were combined. The final mixture was quenched by addition of saturated aqueous NH₄Cl at 0° C. and then extracted with EtOAc (3×). The combined organic layers were dried over Na₂SO₄, filtered, and concentrated. The residue was purified by flash silica gel chromatography (ISCO; 4 g SepaFlash column) using a 0-4% EtOAc/petroleum ether gradient eluent to afford the title compound (110 mg, 12%) as a white solid. ¹H NMR (400 MHz, CDCl₃) δ 7.66 (dd, J=8.00, 6.00 Hz, 1H), 7.49 (d, J=8.13 Hz, 1H), 3.54 (t, J=12.32 Hz, 2H).

### Step 2: 5-Bromo-2,2,4-trifluoro-N-(3-(oxazol-5-yl)-1H-indazol-5-yl)-2,3-dihydro-1H-inden-1-imine

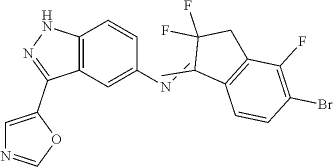

To a solution of 5-bromo-2,2,4-trifluoro-2,3-dihydro-1H-inden-1-one (170 mg, 641.45 umol) 3-oxazol-5-yl-1H-indazol-5-amine (128.42 mg, 641.45 umol) in toluene (3 mL) was added Ti(i-PrO)₄ (911.54 mg, 3.21 mmol) at 20° C. The mixture was then stirred at 120° C. for 13 hrs. The reaction mixture was concentrated to afford the title compound (280 mg, 97%) as a black solid, which was used directly without further purification. MS-ESI (m/z) calc'd for C₁₉H₁₁BrF₃N₄O [M+H]⁺: 447.0/449.0. Found 447.2/449.2.

### Step 3: N-(5-Bromo-2,2,4-trifluoro-2,3-dihydro-1H-inden-1-yl)-3-(oxazol-5-yl)-1H-indazol-5-amine

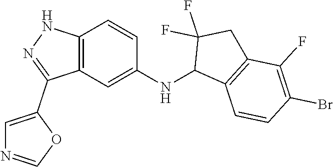 [SMILES: Fc1c(Br)ccc2c1CC(F)(F)C2Nc1ccc2[nH]nc(-c3cnco3)c2c1]

To a solution of N-(5-bromo-2,2,4-trifluoro-2,3-dihydro-1H-inden-1-ylidene)-3-(oxazol-5-yl)-1H-indazol-5-amine (280 mg, 626.11 umol) in MeOH (5 mL) was added NaBH₄ (189.48 mg, 5.01 mmol) at 0° C. and the mixture was stirred at 20° C. for 1 hr. The reaction mixture was quenched by addition of H₂O at 0° C., and then concentrated under reduced pressure to give a residue. The residue was purified by preparative-HPLC using Method FJ to afford the title compound (35 mg, 10%) as a yellow gum TFA salt. MS-ESI (m/z) calc'd for C₁₉H₁₃BrF₃N₄O [M+H]⁺: 449.0/451.0. Found 449.1/451.1.

### Step 4: 2,2,4-Trifluoro-1-((3-(oxazol-5-yl)-1H-indazol-5-yl)amino)-2,3-dihydro-1H-indene-5-carbonitrile

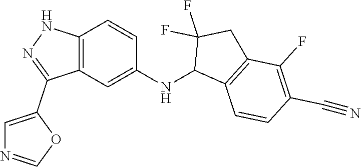 [SMILES: N#Cc1ccc2c(c1F)CC(F)(F)C2Nc1ccc2[nH]nc(-c3cnco3)c2c1]

A mixture of N-(5-bromo-2,2,4-trifluoro-2,3-dihydro-1H-inden-1-yl)-3-(oxazol-5-yl)-1H-indazol-5-amine (35 mg, 77.91 umol), Zn(CN)₂ (27.45 mg, 233.74 umol), Zn (15.28 mg, 233.74 umol), DPPF (4.32 mg, 7.79 umol) and Pd₂(dba)₃ (7.13 mg, 7.79 umol) in DMA (1 mL) was degassed and purged with N₂ (3×) at 20° C. The mixture was then stirred at 100° C. using microwave irradiation for 2 hrs under an N₂ atmosphere. The mixture was filtered; the filtrate was diluted with H₂O and extracted with EtOAc (3×). The combined organic layers were washed with brine (2×), dried over Na₂SO₄, filtered and concentrated give a residue. The residue was purified by preparative-TLC (100% EtOAc, Rf=0.53) to afford the title compound (10 mg, 32%) as a yellow solid. MS-ESI (m/z) calc'd for C₂₀H₁₃F₃N₅O [M+H]⁺: 396.1. Found 396.3.

### Step 5: 2,2,4-Trifluoro-1-((3-(oxazol-5-yl)-1H-indazol-5-yl)amino)-2,3-dihydro-1H-indene-5-carbonitrile, enantiomer 1 and 2

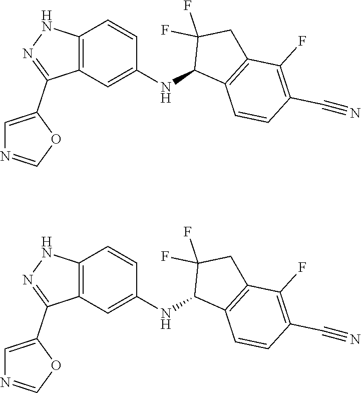 [SMILES: N#Cc1ccc2c(c1F)CC(F)(F)[C@@H]2Nc1ccc2[nH]nc(-c3cnco3)c2c1 ; N#Cc1ccc2c(c1F)CC(F)(F)[C@H]2Nc1ccc2[nH]nc(-c3cnco3)c2c1]

2,2,4-Trifluoro-1-((3-(oxazol-5-yl)-1H-indazol-5-yl)amino)-2,3-dihydro-1H-indene-5-carbonitrile was subjected to chiral separation using Method FK to afford 2,2,4-trifluoro-1-((3-(oxazol-5-yl)-1H-indazol-5-yl)amino)-2,3-dihydro-1H-indene-5-carbonitrile, enantiomer 1 (1.23 mg, 14%) as a red gum. ¹H NMR (400 MHz, DMSO-d₆) δ 13.20 (s, 1H), 8.48 (s, 1H), 7.90 (t, J=7.03 Hz, 1H), 7.67 (s, 1H), 7.44 (d, J=8.92 Hz, 1H), 7.36 (d, J=7.58 Hz, 1H), 7.29 (s, 1H), 7.15 (dd, J=8.99, 1.90 Hz, 1H), 6.40 (d, J=10.02 Hz, 1H), 5.81-5.93 (m, 1H), 3.61-3.72 (m, 2H). MS-ESI (m/z) calc'd for C₂₀H₁₃F₃N₅O [M+H]⁺: 396.1. Found 396.1. A later eluting fraction was also isolated to afford 2,2,4-trifluoro-1-((3-(oxazol-5-yl)-1H-indazol-5-yl)amino)-2,3-dihydro-1H-indene-5-carbonitrile, enantiomer 2 (0.67 mg, 7%) as a red solid. ¹H NMR (400 MHz, DMSO-d₆) δ 13.18 (br s, 1H), 8.48 (d, J=3.18 Hz, 1H), 7.90 (br t, J=7.09 Hz, 1H), 7.66 (d, J=3.06 Hz, 1H), 7.40-7.46 (m, 1H), 7.36 (br d, J=7.82 Hz, 1H), 7.29 (br s, 1H), 7.12-7.18 (m, 1H), 6.38 (br d, J=10.64 Hz, 1H), 5.79-5.91 (m, 1H), 3.67 (br dd, J=17.67, 4.22 Hz, 2H). MS-ESI (m/z) calc'd for C₂₀H₁₃F₃N₅O [M+H]⁺: 396.1. Found 396.1.

### Example 152: 5-((3-(Trifluoromethyl)-1H-indazol-5-yl)oxy)-5,6,7,8-tetrahydronaphthalene-2-carbonitrile, enantiomer 1 and 2

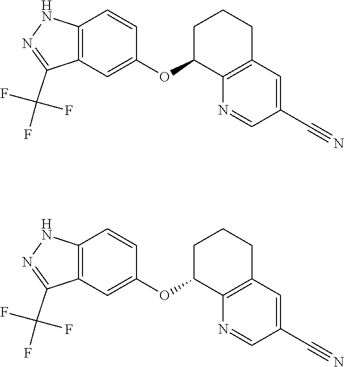 [SMILES: N#Cc1cnc2c(c1)CCC[C@@H]2Oc1ccc2[nH]nc(C(F)(F)F)c2c1 ; N#Cc1cnc2c(c1)CCC[C@H]2Oc1ccc2[nH]nc(C(F)(F)F)c2c1]

### Step 1: 5-Bromo-1-(tetrahydro-2H-pyran-2-yl)-3-(trifluoromethyl)-1H-indazole

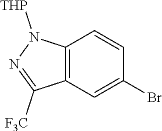

To a solution of 5-bromo-3-(trifluoromethyl)-1H-indazole (500 mg, 1.89 mmol) in DCM (11 mL) were added 3,4-dihydro-2H-pyran (476.08 mg, 5.66 mmol) and PTSA (97.46 mg, 565.97 umol) at 20° C. The mixture was stirred at 30° C. for 2 hrs. The reaction mixture was concentrated and purified by flash silica gel column chromatography (ISCO; 12 g SepaFlash column) using a 0-11% EtOAc/petroleum ether gradient eluent to afford the title compound (570 mg, 86%) as a yellow oil. ¹H NMR (400 MHz, CDCl₃) δ 7.99 (s, 1H), 7.51-7.65 (m, 2H), 5.78 (dd, J=2.63, 8.76 Hz, 1H), 3.92-4.05 (m, 1H), 3.68-3.82 (m, 1H), 2.07-2.21 (m, 2H), 1.65-1.91 (m, 4H).

### Step 2: 1-(Tetrahydro-2H-pyran-2-yl)-5-(4,4,5,5-tetramethyl-1,3,2-dioxaborolan-2-yl)-3-(trifluoromethyl)-1H-indazole

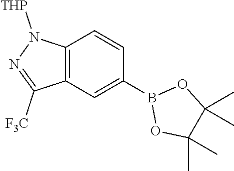

A mixture of 5-bromo-1-(tetrahydro-2H-pyran-2-yl)-3-(trifluoromethyl)-1H-indazole (780 mg, 2.23 mmol), bis(pinacolato)diboron (1.70 g, 6.70 mmol), Pd(dppf)Cl₂ (163.46 mg, 223.40 umol), AcOK (657.76 mg, 6.70 mmol) in dioxane (20 mL) was degassed and purged with N₂ (3×) at 20° C., and then the mixture was stirred at 120° C. for 12 hrs under an N₂ atmosphere. The reaction mixture was diluted with H₂O and extracted with EtOAc (3×). The combined organic phases were dried over anhydrous Na₂SO₄, filtered, and the filtrate was concentrated to give a residue. The residue was purified by flash silica gel column chromatography (ISCO; 12 g SepaFlash column) using a 0-8% EtOAc/petroleum ether gradient eluent to afford the title compound (0.87 g, 98%) as a white solid. ¹H NMR (400 MHz, CDCl₃) δ 8.34 (s, 1H), 7.87 (d, J=8.50 Hz, 1H), 7.65 (d, J=8.50 Hz, 1H), 5.80 (dd, J=2.69, 8.94 Hz, 1H), 3.97-4.06 (m, 1H), 3.71-3.81 (m, 1H), 2.49-2.63 (m, 1H), 2.03-2.23 (m, 2H), 1.65-1.82 (m, 3H), 1.33-1.42 (m, 12H).

### Step 3: 1-(Tetrahydro-2H-pyran-2-yl)-3-(trifluoromethyl)-1H-indazol-5-ol

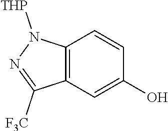

To a solution of 1-(tetrahydro-2H-pyran-2-yl)-5-(4,4,5,5-tetramethyl-1,3,2-dioxaborolan-2-yl)-3-(trifluoromethyl)-1H-indazole (1.24 g, 3.13 mmol) in THF (25 mL) and H₂O (25 mL) was added sodium perborate tetrahydrate (1.44 g, 9.39 mmol) at 20° C. and the mixture was stirred at 50° C. for 1 hr. The reaction mixture was then diluted with H₂O and extracted with EtOAc (3×). The combined organic phases were dried over anhydrous Na₂SO₄, filtered, and the filtrate was concentrated to give a residue. The residue was purified by flash silica gel column chromatography (ISCO; 4 g SepaFlash column) using a 0-10% EtOAc/petroleum ether gradient eluent to afford the title compound (460 mg, 510%) as a red oil. ¹H NMR (400 MHz, CDCl₃) δ 7.55 (d, J=9.13 Hz, 1H), 7.02-7.14 (m, 2H), 5.73 (dd, J=2.63, 9.01 Hz, 1H), 5.33 (br s, 1H), 3.94-4.07 (m, 1H), 3.74 (ddd, J=3.19, 9.85, 11.60 Hz, 1H), 2.42-2.57 (m, 1H), 2.03-2.19 (m, 2H), 1.66-1.81 (m, 3H).

### Step 4: 5-((1-(Tetrahydro-2H-pyran-2-yl)-3-(trifluoromethyl)-1H-indazol-5-yl)oxy)-5,6,7,8-tetrahydronaphthalene-2-carbonitrile

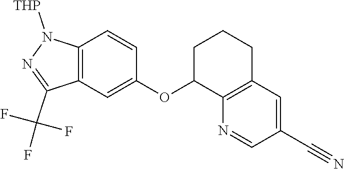

To a solution of 1-(tetrahydro-2H-pyran-2-yl)-3-(trifluoromethyl)-1H-indazol-5-ol (80 mg, 279.48 umol) and 5-hydroxy-5,6,7,8-tetrahydronaphthalene-2-carbonitrile (48.41 mg, 279.48 umol) in toluene (4 mL) was added tributylphosphine (113.09 mg, 558.95 umol) and 1,1-(azodicarbonyl)dipiperidine (141.03 mg, 558.95 umol) at 0° C. The mixture was stirred at 90° C. for 1 hr. The reaction mixture was concentrated and purified by preparative-TLC (SiO₂, petroleum ether/EtOAc=1/1, Rf=0.56) to afford the title compound (102 mg, 82%) as a white oil. ¹H NMR (400 MHz, MeOD) δ 7.75 (d, J=9.13 Hz, 1H), 7.56 (s, 1H), 7.52 (s, 2H), 7.20-7.36 (m, 2H), 5.87 (dd, J=2.69, 9.07 Hz, 1H), 5.49-5.58 (m, 1H), 3.96 (br d, J=11.01 Hz, 1H), 3.75-3.87 (m, 1H), 2.77-3.01 (m, 2H), 2.40-2.55 (m, 1H), 1.99-2.19 (m, 5H), 1.62-1.93 (m, 4H).

### Step 5: 5-((3-(Trifluoromethyl)-1H-indazol-5-yl)oxy)-5,6,7,8-tetrahydronaphthalene-2-carbonitrile

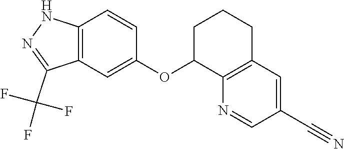 [SMILES: N#Cc1cnc2c(c1)CCCC2Oc1ccc2[nH]nc(C(F)(F)F)c2c1]

To a solution of 5-((1-(tetrahydro-2H-pyran-2-yl)-3-(trifluoromethyl)-1H-indazol-5-yl)oxy)-5,6,7,8-tetrahydronaphthalene-2-carbonitrile (102 mg, 231.06 umol) in DCM (10 mL) was added TFA (4 mL) at 0° C. The mixture was then stirred at 25° C. for 1 hr. The reaction mixture was concentrated and purified by preparative-HPLC using Method FL to afford the title compound (20 mg, 24%) as a white solid. ¹H NMR (400 MHz, MeOD) δ 7.44-7.63 (m, 4H), 7.32 (s, 1H), 7.24 (dd, J=2.06, 9.07 Hz, 1H), 5.51 (br t, J=4.63 Hz, 1H), 3.05 (s, 2H), 2.92 (s, 2H), 2.07 (s, 2H) MS-ESI (m/z) calc'd for C₁₉H₁₅F₃N₃O [M+H]⁺: 358.1. Found 358.0.

### Step 6: 5-((3-(Trifluoromethyl)-1H-indazol-5-yl)oxy)-5,6,7,8-tetrahydronaphthalene-2-carbonitrile, enantiomer 1 and 2

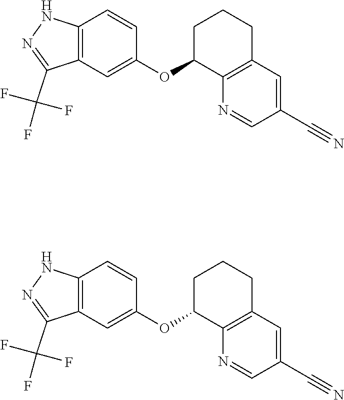 [SMILES: N#Cc1cnc2c(c1)CCC[C@@H]2Oc1ccc2[nH]nc(C(F)(F)F)c2c1 ; N#Cc1cnc2c(c1)CCC[C@H]2Oc1ccc2[nH]nc(C(F)(F)F)c2c1]

5-((3-(Trifluoromethyl)-1H-indazol-5-yl)oxy)-5,6,7,8-tetrahydronaphthalene-2-carbonitrile (9 mg) was subjected to chiral separation using Method FM to afford 5-((3-(trifluoromethyl)-1H-indazol-5-yl)oxy)-5,6,7,8-tetrahydronaphthalene-2-carbonitrile, enantiomer 1 (3.71 mg, 41%) as a colorless gum. ¹H NMR (400 MHz, DMSO-d₆) δ 7.69 (s, 1H), 7.61-7.68 (m, 2H), 7.54-7.60 (m, 1H), 7.37 (s, 1H), 7.25 (dd, J=2.21, 9.04 Hz, 1H), 5.59-5.69 (m, 1H), 2.85-2.96 (m, 1H), 2.72-2.83 (m, 1H), 2.01 (q, J=5.29 Hz, 2H), 1.77-1.92 (m, 2H). MS-ESI (m/z) calc'd for C₁₉H₁₅F₃N₃O [M+H]⁺: 358.1. Found 358.2. A later eluting fraction was also isolated to afford 5-((3-(trifluoromethyl)-1H-indazol-5-yl)oxy)-5,6,7,8-tetrahydronaphthalene-2-carbonitrile, enantiomer 2 (3.87 mg, 43%) as a colorless gum. ¹H NMR (400 MHz, DMSO-d₆) δ 13.90 (br s, 1H), 7.70 (s, 1H), 7.63-7.68 (m, 2H), 7.54-7.58 (m, 1H), 7.37 (s, 1H), 7.26 (dd, J=2.09, 9.15 Hz, 1H), 5.65 (t, J=4.74 Hz, 1H), 2.85-2.95 (m, 1H), 2.72-2.83 (m, 1H), 1.96-2.07 (m, 2H), 1.77-1.93 (m, 2H). MS-ESI (m/z) calc'd for C₁₉H₁₅F₃N₃O [M+H]⁺: 358.1. Found 358.2.

### Example 153: 5-((3-(Trifluoromethyl)-1H-indazol-5-yl)amino)-5,6,7,8-tetrahydronaphthalene-2-carbonitrile, enantiomer 1 and 2

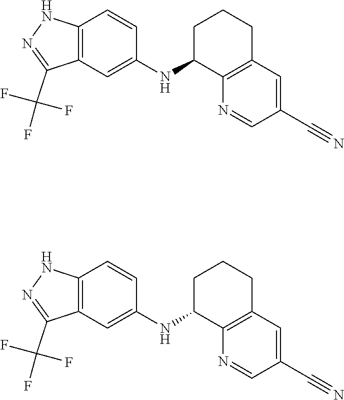 [SMILES: N#Cc1cnc2c(c1)CCC[C@@H]2Nc1ccc2[nH]nc(C(F)(F)F)c2c1 ; N#Cc1cnc2c(c1)CCC[C@H]2Nc1ccc2[nH]nc(C(F)(F)F)c2c1]

### Step 1: 5-((1-Tetrahydro-2H-pyran-2-yl)-3-(trifluoromethyl)-1H-indazol-5-yl)amino)-5,6,7,8-tetrahydronaphthalene-2-carbonitrile

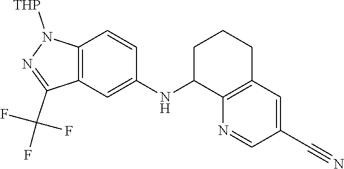

To a solution of 5-bromo-1-(tetrahydro-2H-pyran-2-yl)-3-(trifluoromethyl)-1H-indazole (100 mg, 286.41 umol) in THF (1 mL) were added 5-amino-5,6,7,8-tetrahydronaphthalene-2-carbonitrile (98.66 mg, 572.83 umol), t-BuXPhos Pd G3 (22.75 mg, 28.64 umol), and t-BuONa (55.05 mg, 572.83 umol). The mixture was stirred at 60° C. for 6 hrs under an N₂ atmosphere. The reaction mixture was concentrated and purified by preparative-TLC (petroleum ether/EtOAc=2/1, Rf=0.43) to afford the title compound (100 mg, 79%) as a white solid.

### Step 2: 5-((3-(Trifluoromethyl)-1H-indazol-5-yl)amino)-5,6,7,8-tetrahydronaphthalene-2-carbonitrile

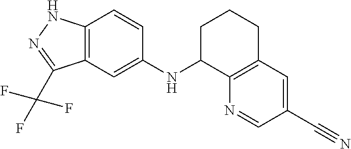 [SMILES: N#Cc1cnc2c(c1)CCCC2Nc1ccc2[nH]nc(C(F)(F)F)c2c1]

To a solution of 5-((1-(tetrahydro-2H-pyran-2-yl)-3-(trifluoromethyl)-1H-indazol-5-yl)amino)-5,6,7,8-tetrahydronaphthalene-2-carbonitrile (100 mg, 227.04 umol) in CH₂Cl₂ (4 mL) was added TFA (1.54 g, 13.51 mmol). The mixture was stirred at 20° C. for 3 hrs and then concentrated and purified by preparative-HPLC using Method FN to afford the title compound (35 mg, 33%) as a yellow solid TFA salt. MS-ESI (m/z) calc'd for C₁₉H₁₆F₃N₄. [M+H]⁺:357.1. Found 357.2.

### Step 3: 5-((3-(Trifluoromethyl)-1H-indazol-5-yl)amino)-5,6,7,8-tetrahydronaphthalene-2-carbonitrile, enantiomer 1 and 2

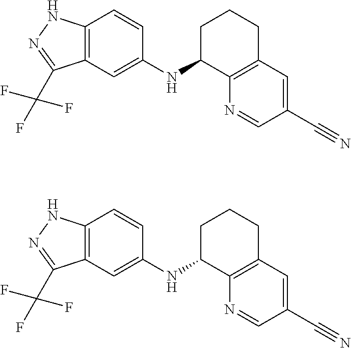 [SMILES: N#Cc1cnc2c(c1)CCC[C@@H]2Nc1ccc2[nH]nc(C(F)(F)F)c2c1 ; N#Cc1cnc2c(c1)CCC[C@H]2Nc1ccc2[nH]nc(C(F)(F)F)c2c1]

5-((3-(Trifluoromethyl)-1H-indazol-5-yl)amino)-5,6,7,8-tetrahydronaphthalene-2-carbonitrile (9 mg) was subjected to chiral separation using Method FO to afford 5-((3-(trifluoromethyl)-1H-indazol-5-yl)amino)-5,6,7,8-tetrahydronaphthalene-2-carbonitrile, enantiomer 1 (3.78 mg, 42%) as a white solid. ¹H NMR (400 MHz, DMSO-d₆) δ 7.65 (s, 1H), 7.57-7.61 (m, 1H), 7.44-7.52 (m, 2H), 7.07 (dd, J=9.07, 1.94 Hz, 1H), 6.75 (s, 1H), 6.16 (d, J=8.76 Hz, 1H), 4.71 (br d, J=8.63 Hz, 1H), 2.78-2.86 (m, 2H), 1.79-1.98 (m, 4H). MS-ESI (m/z) calc'd for C₁₉H₁₆F₃N₄ [M+H]⁺: 357.1. Found 357.1. A later eluting fraction was also isolated to afford 5-((3-(trifluoromethyl)-1H-indazol-5-yl)amino)-5,6,7,8-tetrahydronaphthalene-2-carbonitrile, enantiomer 2 (4.02 mg, 45%) as a white solid. ¹H NMR (400 MHz, DMSO-d₆) δ 7.65 (s, 1H) 7.57-7.61 (m, 1H) 7.44-7.52 (m, 2H) 7.07 (dd, J=9.07, 1.94 Hz, 1H) 6.75 (s, 1H) 6.16 (d, J=8.88 Hz, 1H) 4.71 (br d, J=8.00 Hz, 1H) 2.76-2.86 (m, 2H) 1.78-2.00 (m, 4H). MS-ESI (m/z) calc'd for C₁₉H₁₆F₃N₄ [M+H]⁺:357.1. Found 357.1.

### Example 154: 4-Methoxy-7-((3-(oxazol-5-yl)-1H-indazol-5-yl)amino)-6,7-dihydro-5H-cyclopenta[b]pyridine-3-carbonitrile, enantiomer 1 and 2

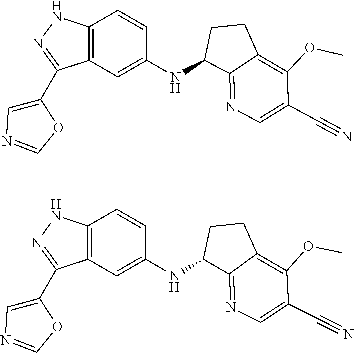 [SMILES: COc1c(C#N)cnc2c1CC[C@@H]2Nc1ccc2[nH]nc(-c3cnco3)c2c1 ; COc1c(C#N)cnc2c1CC[C@H]2Nc1ccc2[nH]nc(-c3cnco3)c2c1]

### Step 1: 4-Methoxy-6,7-dihydro-5H-cyclopenta[b]pyridine

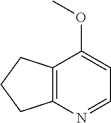

To a solution of 4-chloro-6,7-dihydro-5H-cyclopenta[b]pyridine (2 g, 13.02 mmol) in MeOH (10 mL) was added NaOMe (2.18 g, 40.36 mmol) at 20° C. The mixture was stirred at 110° C. for 12 hrs in sealed tube. The mixture was filtered and the filtrate was concentrated to give a residue. The residue was purified by flash silica gel chromatography (ISCO; 20 g SepaFlash column) using a 0-15% EtOAc/petroleum ether gradient eluent to afford the title compound (1.84 g, 95%) as a pale yellow oil. ¹H NMR (400 MHz, CDCl₃) δ 8.27 (d, J=5.70 Hz, 1H), 6.58 (d, J=5.70 Hz, 1H), 3.86 (s, 3H), 3.00 (t, J=7.67 Hz, 2H), 2.87 (t, J=7.34 Hz, 2H), 2.10 (quin, J=7.62 Hz, 2H). MS-ESI (m/z) calc'd for C₉H₁₂NO [M+H]⁺: 150.1. Found 150.1.

### Step 2: 3-Bromo-4-methoxy-6,7-dihydro-5H-cyclopenta[b]pyridine

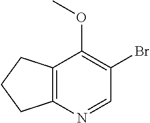

To a solution of 4-methoxy-6,7-dihydro-5H-cyclopenta[b]pyridine (1.9 g, 12.74 mmol) in conc. H₂SO₄ (18 mL) was added NBS (2.95 g, 16.56 mmol) at 0° C. The mixture was stirred at 60° C. for 3 hrs. The reaction mixture was poured into H₂O and adjusted to pH=10 with 2 M aqueous NaOH, then extracted with EtOAc (3×). The combined organic layers were washed with brine (3×), dried over Na₂SO₄, and filtered. The filtrate was concentrated and purified by flash silica gel chromatography (ISCO; 12 g SepaFlash column) using a 0-19% EtOAc/petroleum ether gradient eluent to afford the title compound (2 g, 69%) as a pale yellow gum. ¹H NMR (400 MHz, DMSO-d₆) δ 8.30 (s, 1H), 4.03 (s, 3H), 3.21 (t, J=7.34 Hz, 2H), 2.82 (t, J=7.78 Hz, 2H), 1.97-2.10 (m, 2H). MS-ESI (m/z) calc'd for C₉H₁₁BrNO [M+H]⁺: 228.0/230.0. Found 228.2/230.2.

### Step 3: 3-Bromo-4-methoxy-6,7-dihydro-5H-cyclopenta[b]pyridine 1-oxide

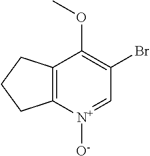

To a solution of 3-bromo-4-methoxy-6,7-dihydro-5H-cyclopenta[b]pyridine (1.5 g, 6.58 mmol) in DCM (20 mL) was added m-CPBA (2.00 g, 9.86 mmol). The mixture was stirred at 20° C. for 12 hrs and then stirred at 50° C. for an additional 2 hrs. The reaction mixture was quenched by addition 10% aqueous Na₂SO₃ and extracted with EtOAc (3×). The combined organic layers were dried over Na₂SO₄, filtered, and concentrated to afford the title compound (1.5 g, 93%) as a yellow solid. MS-ESI (m/z) calc'd for C₉H₁₁BrNO₂ [M+H]⁺: 244.0/246.0. Found 244.1/246.1.

### Step 4: 3-Bromo-4-methoxy-6,7-dihydro-5H-cyclopenta[b]pyridin-7-yl acetate

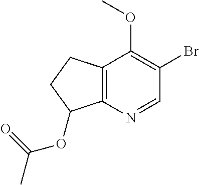 [SMILES: COc1c(Br)cnc2c1CCC2OC(C)=O]

A solution of 3-bromo-4-methoxy-6,7-dihydro-5H-cyclopenta[b]pyridine 1-oxide (1.5 g, 6.15 mmol) in Ac₂O (7.21 g, 70.67 mmol) was stirred at 100° C. for 2 hrs. The reaction mixture was adjusted to pH=8 with saturated aqueous NaHCO₃ and then extracted with EtOAc (3×). The combined organic layers were dried over Na₂SO₄, filtered, and concentrated to afford the title compound (1.5 g, 85%) as a brown oil. MS-ESI (m/z) calc'd for C₁₁H₁₃BrNO₃ [M+H]⁺: 286.0/288.0. Found 286.2/288.2.

### Step 5: 3-Bromo-4-methoxy-6,7-dihydro-5H-cyclopenta[b]pyridin-7-ol

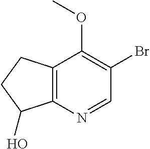

To a solution of 3-bromo-4-methoxy-6,7-dihydro-5H-cyclopenta[b]pyridin-7-yl acetate (1.5 g, 5.24 mmol) in MeOH (15 mL) was added K₂CO₃ (2.90 g, 20.97 mmol) at 20° C. The mixture was stirred at 20° C. for 0.5 hr and then concentrated to give a residue. The residue was diluted with H₂O and extracted with EtOAc (3×). The combined organic layers were dried over Na₂SO₄, filtered, and the filtrate was concentrated and purified by preparative-TLC (100% EtOAc, Rf=0.3) to afford the title compound (547.8 mg, 43%) as a yellow solid. MS-ESI (m/z) calc'd for C₉H₁₁BrNO₂ [M+H]⁺: 244.0/246.0. Found 244.2/246.2.

### Step 6: 3-Bromo-4-methoxy-5H-cyclopenta[b]pyridin-7(6H)-one

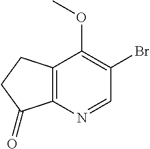

To a solution of 3-bromo-4-methoxy-6,7-dihydro-5H-cyclopenta[b]pyridin-7-ol (120 mg, 491.63 umol) in DCM (2 mL) was added Dess-Martin periodinane (312.78 mg, 737.45 umol) at 20° C. and the mixture was stirred at for 12 hrs. The mixture was adjusted to pH=8 with saturated aqueous Na₂CO₃, and then extracted with EtOAc (3×). The combined organic layers were dried over Na₂SO₄, filtered, and concentrated to give a residue. The residue was purified by preparative-TLC (100% EtOAc, Rf=0.48) to afford the title compound (40 mg, 34%) as a yellow solid. MS-ESI (m/z) calc'd for C₉H₉BrNO₂ [M+H]⁺: 242.0/244.0. Found 242.1/244.1.

### Step 7: N-(3-Bromo-4-methoxy-6,7-dihydro-5H-cyclopenta[b]pyridin-7-yl)-3-(oxazol-5-yl)-1H-indazol-5-amine

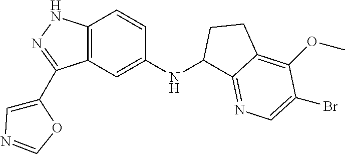 [SMILES: COc1c(Br)cnc2c1CCC2Nc1ccc2[nH]nc(-c3cnco3)c2c1]

To a solution of 3-bromo-4-methoxy-5H-cyclopenta[b]pyridin-7(6R)-one (40 mg, 165.24 umol) and 3-(oxazol-5-yl)-1H-indazol-5-amine (33.08 mg, 165.24 umol) in MeOH (3 mL) was added AcOH (19.85 mg, 330.48 umol) and the mixture was stirred at 20° C. for 2 hrs. Then NaBH₃CN (31.15 mg, 495.73 umol) was added and the mixture was stirred at 20° C. for an additional 2 hrs. The reaction mixture was concentrated and purified by preparative-TLC (100% EtOAc, Rf=0.35) to afford the title compound (22.1 mg, 31%) as a yellow solid. MS-ESI (m/z) calc'd for C₁₉H₁₇BrN₅O₂ [M+H]⁺: 426.1/428.1. Found 426.2/428.2.

### Step 8: 4-Methoxy-7-((3-(oxazol-5-yl)-1H-indazol-5-yl)amino)-6,7-dihydro-5H-cyclopenta[b]pyridine-3-carbonitrile

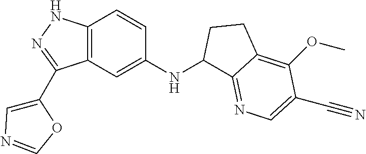 [SMILES: COc1c(C#N)cnc2c1CCC2Nc1ccc2[nH]nc(-c3cnco3)c2c1]

A mixture of N-(3-bromo-4-methoxy-6,7-dihydro-5H-cyclopenta[b]pyridin-7-yl)-3-(oxazol-5-yl)-1H-indazol-5-amine (50 mg, 117.30 umol), Zn(CN)₂ (27.55 mg, 234.60 umol), Zn (15.34 mg, 234.60 umol), DPPF (13.01 mg, 23.46 umol) and Pd₂(dba)₃ (21.48 mg, 23.46 umol) in DMA (2 mL) was degassed and purged with N₂ (3×) at 20° C. and the mixture was then stirred at 100° C. for 2 hrs under an N₂ atmosphere. The reaction mixture was concentrated and purified by preparative-HPLC using Method FP to afford the title compound (9 mg, 16%) as a yellow solid, TFA salt. MS-ESI (m/z) calc'd for C₂₀H₁₇N₆O₂ [M+H]⁺: 373.1. Found 373.3.

### Step 9: 4-Methoxy-7-((3-(oxazol-5-yl)-1H-indazol-5-yl)amino)-6,7-dihydro-5H-cyclopenta[b]pyridine-3-carbonitrile

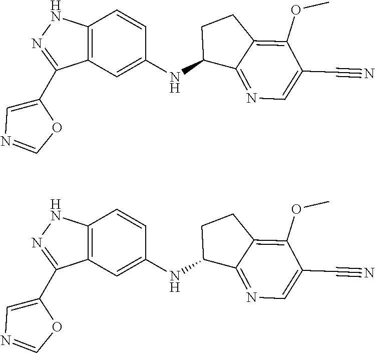 [SMILES: COc1c(C#N)cnc2c1CC[C@@H]2Nc1ccc2[nH]nc(-c3cnco3)c2c1 ; COc1c(C#N)cnc2c1CC[C@H]2Nc1ccc2[nH]nc(-c3cnco3)c2c1]

4-Methoxy-7-((3-(oxazol-5-yl)-1H-indazol-5-yl)amino)-6,7-dihydro-5H-cyclopenta[b]pyridine-3-carbonitrile was subjected to chiral separation using Method FQ to afford 4-methoxy-7-((3-(oxazol-5-yl)-1H-indazol-5-yl)amino)-6,7-dihydro-5H-cyclopenta[b]pyridine-3-carbonitrile, enantiomer 1 (3.49 mg, 39%) as a gray solid. ¹H NMR (400 MHz, DMSO-d₆) δ 13.11 (s, 1H), 8.64 (s, 1H), 8.47 (s, 1H), 7.65 (s, 1H), 7.37 (d, J=8.99 Hz, 1H), 7.13 (s, 1H), 7.02 (dd, J=8.99, 1.75 Hz, 1H), 5.94 (d, J=7.02 Hz, 1H), 4.99 (q, J=7.45 Hz, 1H), 4.24 (s, 3H), 3.36-3.43 (m, 1H), 3.15-3.26 (m, 1H), 2.60-2.69 (m, 1H), 1.82-1.96 (m, 1H). MS-ESI (m/z) calc'd for C₂₀H₁₇N₆O₂ [M+H]⁺: 373.1. Found 373.1. A later eluting fraction was also isolated to afford 4-methoxy-7-((3-(oxazol-5-yl)-1H-indazol-5-yl)amino)-6,7-dihydro-5H-cyclopenta[b]pyridine-3-carbonitrile, enantiomer 2 (2.78 mg, 31%) as a green gum. ¹H NMR (400 MHz, DMSO-d₆) δ 8.64 (s, 1H), 8.47 (s, 1H), 7.65 (s, 1H), 7.37 (d, J=8.77 Hz, 1H), 7.12 (s, 1H), 7.01 (dd, J=8.88, 1.86 Hz, 1H), 5.94 (d, J=6.80 Hz, 1H), 4.96-5.03 (m, 1H), 4.24 (s, 3H), 3.37-3.42 (m, 1H), 3.16-3.25 (m, 1H), 2.59-2.69 (m, 1H), 1.85-1.96 (m, 1H). MS-ESI (m/z) calc'd for C₂₀H₁₇N₆O₂ [M+H]⁺: 373.1. Found 373.2.

### Example 155: 2,2-Difluoro-1-((3-(2-methyloxazol-5-yl)-1H-indazol-5-yl)amino)-2,3-dihydro-1H-indene-5-carbonitrile, enantiomer 1 and 2

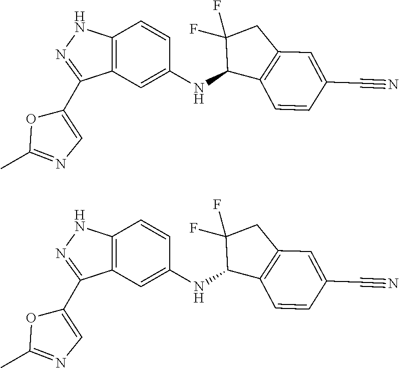 [SMILES: Cc1ncc(-c2n[nH]c3ccc(N[C@@H]4c5ccc(C#N)cc5CC4(F)F)cc23)o1 ; Cc1ncc(-c2n[nH]c3ccc(N[C@H]4c5ccc(C#N)cc5CC4(F)F)cc23)o1]

### Step 1: 3-(1-Ethoxyvinyl)-5-nitro-1-((2-(trimethylsilyl)ethoxy)methyl)-1H-indazole

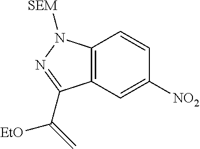 [SMILES: C=C(OCC)c1nn(COCC[Si](C)(C)C)c2ccc([N+](=O)[O-])cc12]

A mixture of 3-iodo-5-nitro-1-((2-(trimethylsilyl)ethoxy)methyl)-1H-indazole (4.5 g, 10.74 mmol), tributyl(1-ethoxyvinyl)stannane (4.66 g, 12.88 mmol), Pd(PPh₃)₄ (1.24 g, 1.07 mmol) in toluene (80 mL) was degassed and purged with N₂ (3×) at 25° C. The mixture was then stirred at 100° C. for 12 hrs under an N₂ atmosphere. The mixture was concentrated and purified by flash silica gel column chromatography (ISCO; 20 g SepaFlash column) using a 0-6% EtOAc/petroleum ether gradient eluent to afford the title compound (3.12 g, 80%) as a yellow oil. ¹H NMR (400 MHz, CDCl₃) δ 7.96-8.06 (m, 1H), 7.60-7.66 (m, 1H), 7.37-7.46 (m, 1H), 5.84-5.91 (m, 2H), 4.81-4.85 (m, 1H), 4.71 (d, J=2.88 Hz, 1H), 4.17 (q, J=7.13 Hz, 1H), 4.04-4.10 (m, 1H), 3.66-3.72 (m, 2H), 1.26-1.36 (m, 3H), 0.96-0.99 (m, 2H), 0.00-0.01 (m, 9H). MS-ESI (m/z) calc'd for C₁₇H₂₆N₃₀₄Si [M+H]⁺: 364.2. Found 364.3.

### Step 2: 1-(5-Nitro-1-((2-(trimethylsilyl)ethoxy)methyl)-1H-indazol-3-yl)ethanone

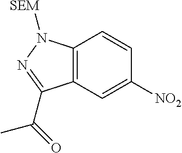 [SMILES: CC(=O)c1nn(COCC[Si](C)(C)C)c2ccc([N+](=O)[O-])cc12]

To a solution of 3-(1-ethoxyvinyl)-5-nitro-1-((2-(trimethylsilyl)ethoxy)methyl)-1H-indazole (2.8 g, 7.7 mmol) in THF (40 mL) was added HCl (2 M, 9.34 mL) at 25° C. The mixture was stirred at 25° C. for 30 minutes and then diluted with H₂O and extracted with EtOAc (3×). The combined organic phases were dried over anhydrous Na₂SO₄, filtered, and concentrated. The residue was purified by flash silica gel column chromatography (ISCO; 12 g SepaFlash column) using a 0-2% EtOAc/petroleum ether gradient eluent to afford the title compound (1.76 g, 68%) as a yellow solid. ¹H NMR (400 MHz, DMSO-d₆) δ 9.01 (d, J=2.20 Hz, 1H), 8.40 (dd, J=9.29, 2.20 Hz, 1H), 8.15 (d, J=9.17 Hz, 1H), 5.99 (s, 2H), 3.61-3.66 (m, 2H), 2.71 (s, 3H), 0.83-0.89 (m, 2H), −0.09-−0.05 (m, 9H).

### Step 3: 2-Methyl-5-(5-nitro-1-((2-(trimethylsilyl)ethoxy)methyl)-1H-indazol-3-yl)oxazole

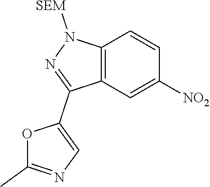 [SMILES: Cc1ncc(-c2nn(COCC[Si](C)(C)C)c3ccc([N+](=O)[O-])cc23)o1]

To a solution of 1-(5-nitro-1-((2-(trimethylsilyl)ethoxy)methyl)-1H-indazol-3-yl)ethanone (1.4 g, 4.17 mmol) in DMSO (25 mL) was added 12 (2.12 g, 8.35 mmol) at 25° C. and the mixture was stirred at 110° C. for 45 minutes. Then 2-aminopropanoic acid (743.70 mg, 8.35 mmol) was added and the mixture was stirred at 110° C. for an additional 15 minutes. The reaction mixture was diluted with H₂O and extracted with EtOAc (3×). The combined organic phases were dried over anhydrous Na₂SO₄, filtered, and concentrated. The residue was purified by flash silica gel column chromatography (ISCO; 20 g SepaFlash column) using a 0-16% EtOAc/petroleum ether gradient eluent to afford the title compound (920 mg, 59%) as a yellow solid. ¹H NMR (400 MHz, CDCl₃) δ 9.01 (d, J=1.83 Hz, 1H), 8.41 (dd, J=9.23, 2.02 Hz, 1H), 7.74 (d, J=9.17 Hz, 1H), 7.64 (s, 1H), 5.85 (s, 2H), 3.64 (d, J=8.44 Hz, 2H), 2.72 (s, 3H), 0.95 (d, J=8.19 Hz, 2H), −0.02 (s, 9H). MS-ESI (m/z) calc'd for C₁₇H₂₃N₄O₄Si [M+H]⁺: 375.1. Found 375.2.

### Step 4: 3-(2-Methyloxazol-5-yl)-1-((2-(trimethylsilyl)ethoxy)methyl)-1H-indazol-5-amine

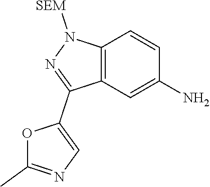 [SMILES: Cc1ncc(-c2nn(COCC[Si](C)(C)C)c3ccc(N)cc23)o1]

To a solution of 2-methyl-5-(5-nitro-1-((2-(trimethylsilyl)ethoxy)methyl)-1H-indazol-3-yl)oxazole (1.09 g, 2.91 mmol) in EtOH (20 mL) and H₂O (20 mL) was added Fe (812.85 mg, 14.55 mmol) and NH₄Cl (778.50 mg, 14.55 mmol) at 20° C. and the mixture was then stirred at 80° C. for 2 hrs. The reaction mixture was filtered and the filtrate was concentrated to give a residue. The residue was diluted with H₂O and extracted with EtOAc (3×). The combined organic phases were dried over anhydrous Na₂SO₄, filtered, and concentrated to afford the title compound (800 mg, 80%) as a red gum. ¹H NMR (400 MHz, CDCl₃) δ 7.47 (br d, J=7.95 Hz, 1H), 7.43 (br s, 1H), 7.33 (br s, 1H), 7.12 (br s, 1H), 5.63-5.86 (m, 2H), 3.60 (br d, J=7.21 Hz, 2H), 2.53-2.72 (m, 3H), 2.04-2.23 (m, 2H), 0.92 (br d, J=6.97 Hz, 2H), −0.04 (br s, 9H). MS-ESI (m/z) calc'd for C₁₇H₂₅N₄₀₂Si [M+H]⁺: 345.2. Found 345.3.

### Step 5: 5-Bromo-2-fluoro-2,3-dihydro-1H-inden-1-one

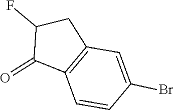 [SMILES: O=C1c2ccc(Br)cc2CC1F]

To a solution of 5-bromo-2,3-dihydro-1H-inden-1-one (3 g, 14.21 mmol) in MeOH (30 mL) was added 1-chloromethyl-4-fluoro-1,4-diazoniabicyclo[2.2.2]octane bis(tetrafluoroborate) (6.04 g, 17.06 mmol) at 25° C. The mixture was stirred at 70° C. for 2 hrs. The reaction was filtered and the filtrate was concentrated to give a residue. The residue was dissolved in THF and 1 N HCl was added, followed by stirring at room temperature for 3 hrs. Then 2 N aqueous NaOH was added to the mixture to adjust to pH=9 and the mixture was extracted with EtOAc (3×). The combined organic phases were dried over anhydrous Na₂SO₄, filtered, and concentrated to afford the title compound (3.2 g, 98%) as a yellow solid. ¹H NMR (400 MHz, CDCl₃) δ 7.61 (s, 1H), 7.50 (br d, J=2.38 Hz, 1H), 7.43-7.47 (m, 1H), 5.17-5.39 (m, 1H), 3.12-3.28 (m, 2H). MS-ESI (m/z) calc'd for C₉H₇BrFO [M+H]⁺: 229.0/231.0. Found 229.1/231.1.

### Step 6: 5-Bromo-2,2-difluoro-2,3-dihydro-1H-inden-1-one

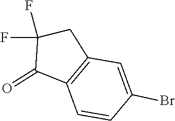 [SMILES: O=C1c2ccc(Br)cc2CC1(F)F]

To a solution of 5-bromo-2-fluoro-2,3-dihydro-1H-inden-1-one (3.2 g, 13.97 mmol) in CH₂Cl₂ (64 mL) was added Et₃N (6.90 g, 68.18 mmol) and [tert-butyl(dimethyl)silyl]trifluoromethanesulfonate (5.50 g, 20.82 mmol) at 25° C. The mixture was stirred at 25° C. for 1 hr. The reaction mixture was diluted with a saturated aqueous NaHCO₃, and extracted with CH₂Cl₂ (3×). The combined organic phases were dried over anhydrous Na₂SO₄, the mixture was filtered and the filtrate was concentrated to give a residue. The residue was dissolved in ACN (64 mL), and Select F (5.94 g, 16.77 mmol) was added at 25° C., and then the mixture was stirred at 25° C. for 2 hrs. The reaction mixture was diluted with H₂O and extracted with EtOAc (3×). The combined organic phases were dried over anhydrous Na₂SO₄, the mixture was filtered and the filtrate was concentrated under vacuum. The residue was purified by flash silica gel column chromatography (ISCO; 12 g SepaFlash column) using a 0-1% EtOAc/petroleum ether gradient eluent to afford the title compound (2.1 g, 60%) as a yellow oil. ¹H NMR (400 MHz, CDCl₃) δ 7.63-7.68 (m, 1H), 7.55-7.62 (m, 2H), 3.48 (t, J=12.57 Hz, 2H).

### Step 7: 2,2-Difluoro-1-oxo-2,3-dihydro-1H-indene-5-carbonitrile

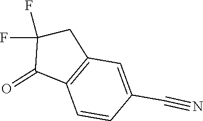 [SMILES: N#Cc1ccc2c(c1)CC(F)(F)C2=O]

A mixture of 5-bromo-2,2-difluoro-2,3-dihydro-1H-inden-1-one (400 mg, 1.62 mmol), Zn(CN)₂ (570.40 mg, 4.86 mmol), Zn (317.64 mg, 4.86 mmol), 1,1-bis(diphenylphosphino)ferrocene (89.77 mg, 161.92 umol) and Pd₂dba₃ (148.27 mg, 161.92 umol) in DMA (20 mL) was degassed and purged with N₂ (3×) at 25° C., and then the mixture was stirred under an N₂ atmosphere at 100° C. using microwave irradiation for 2 hrs. This procedure was repeated and the reaction mixtures were combined. The combined mixture was filtered and the filtrate was concentrated to give a residue. The residue was diluted with H₂O and extracted with EtOAc (3×). The combined organic phases were dried over anhydrous Na₂SO₄ and filtered. The filtrate was evaporated and purified by flash silica gel column chromatography (ISCO; 20 g SepaFlash column) using a 0-12% EtOAc/petroleum ether gradient eluent to afford the title compound (470 mg, 75%) as a yellow solid. ¹H NMR (400 MHz, CDCl₃) δ 8.02 (d, J=7.95 Hz, 1H), 7.85 (s, 1H), 7.82 (d, J=7.95 Hz, 1H), 3.56-3.76 (m, 2H).

### Step 8: 2,2-Difluoro-1-((3-(2-methyloxazol-5-yl)-1-((2-(trimethylsilyl)ethoxy)methyl)-1H-indazol-5-yl)amino)-2,3-dihydro-1H-indene-5-carbonitrile trifluoroacetate

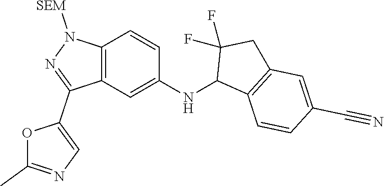 [SMILES: Cc1ncc(-c2nn(COCC[Si](C)(C)C)c3ccc(NC4c5ccc(C#N)cc5CC4(F)F)cc23)o1]

To a solution of 3-(2-methyloxazol-5-yl)-1-((2-(trimethylsilyl)ethoxy)methyl)-1H-indazol-5-amine (20 mg, 58.06 umol) in toluene (1 mL) were added 2,2-difluoro-1-oxo-2,3-dihydro-1H-indene-5-carbonitrile (22.43 mg, 116.12 umol) and Ti(i-PrO)₄ (132.01 mg, 464.46 umol) at 20° C. The mixture was then stirred at 100° C. for 12 hrs and concentrated to give a residue. Then the residue was dissolved in MeOH (1 mL) and NaBH₄ (17.47 mg, 461.88 umol) was added at 0° C. The mixture was stirred at 20° C. for 2 hrs. This procedure was conducted seven times and the reaction mixtures were combined. The combined mixture was poured into ice water and filtered. The filtrate was evaporated to remove MeOH and then extracted with EtOAc (3×). The combined organic phases were dried over anhydrous Na₂SO₄, and filtered, the filtrate was concentrated and purified by preparative-HPLC using Method FR to afford the title compound (60 mg, 23%) as a yellow so lid, TFA salt. MS-ESI (m/z) calc'd for C₂₇H₃₀F₂N₅O₂Si [M+H]⁺: 522.2. Found 522.4.

### Step 9: 2,2-Difluoro-1-((1-(hydroxymethyl)-3-(2-methyloxazol-5-yl)-1H-indazol-5-yl)amino)-2,3-dihydro-1H-indene-5-carbonitrile

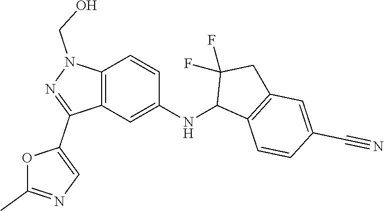 [SMILES: Cc1ncc(-c2nn(CO)c3ccc(NC4c5ccc(C#N)cc5CC4(F)F)cc23)o1]

A solution of 2,2-difluoro-1-((3-(2-methyloxazol-5-yl)-1-((2-(trimethylsilyl)ethoxy)methyl)-1H-indazol-5-yl)amino)-2,3-dihydro-1H-indene-5-carbonitrile trifluoroacetate (60 mg, 94.39 umol) in TFA (1 mL) was stirred at 20° C. for 2 hrs. The reaction mixture was basified with saturated aqueous NaHCO₃ to pH=8 and extracted with EtOAc (3×). The combined organic layers were dried over Na₂SO₄, filtered, and concentrated to afford the title compound (50 mg) as a green solid which was used without further purification. MS-ESI (m/z) calc'd for C₂₂H₁₈F₂N₅O₂ [M+H]⁺: 422.1. Found 422.3.

### Step 10: 2,2-Difluoro-1-((3-(2-methyloxazol-5-yl)-1H-indazol-5-yl)amino)-2,3-dihydro-1H-indene-5-carbonitrile

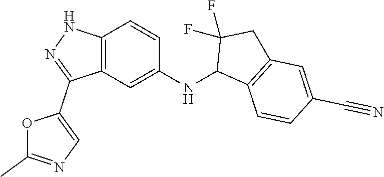 [SMILES: Cc1ncc(-c2n[nH]c3ccc(NC4c5ccc(C#N)cc5CC4(F)F)cc23)o1]

A solution of 2,2-difluoro-1-((1-(hydroxymethyl)-3-(2-methyloxazol-5-yl)-1H-indazol-5-yl)amino)-2,3-dihydro-1H-indene-5-carbonitrile (50 mg, 118.65 umol) in dioxane (1.2 mL) and NH₄OH (0.3 mL) was stirred at 20° C. for 2 hrs. The reaction mixture was diluted with H₂O and extracted with EtOAc (3×). The combined organic phases were dried over anhydrous Na₂SO₄, filtered, and the filtrate was concentrated to give a residue. The residue was purified by preparative-HPLC using Method FS to afford the title compound (22 mg, 47%) as a pink solid. MS-ESI (m/z) calc'd for C₂₁H₁₆F₂N₅O [M+H]⁺: 392.1. Found 392.0.

### Step 11: 2,2-Difluoro-1-((3-(2-methyloxazol-5-yl)-1H-indazol-5-yl)amino)-2,3-dihydro-1H-indene-5-carbonitrile, enantiomer 1 and 2

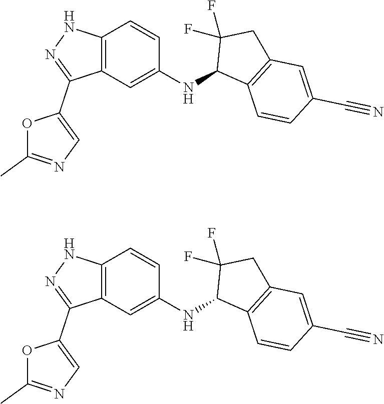 [SMILES: Cc1ncc(-c2n[nH]c3ccc(N[C@@H]4c5ccc(C#N)cc5CC4(F)F)cc23)o1 ; Cc1ncc(-c2n[nH]c3ccc(N[C@H]4c5ccc(C#N)cc5CC4(F)F)cc23)o1]

2,2-Difluoro-1-((3-(2-methyloxazol-5-yl)-1H-indazol-5-yl)amino)-2,3-dihydro-1H-indene-5-carbonitrile (9 mg) was subjected to chiral separation using Method FT to afford 2,2-difluoro-1-((3-(2-methyloxazol-5-yl)-1H-indazol-5-yl)amino)-2,3-dihydro-1H-indene-5-carbonitrile, enantiomer 1 (3.47 mg, 38%) as a yellow solid. ¹H NMR (400 MHz, MeOH-d₄) δ 7.68-7.74 (m, 2H), 7.60 (d, J=7.82 Hz, 1H), 7.42-7.49 (m, 2H), 7.34 (s, 1H), 7.21 (dd, J=8.99, 2.02 Hz, 1H), 5.55-5.64 (m, 1H), 3.49-3.71 (m, 2H), 2.60 (s, 3H). MS-ESI (m/z) calc'd for C₂₁H₁₆F₂N₅O [M+H]⁺: 392.1. Found 392.1. A later eluting fraction was also isolated to afford 2,2-difluoro-1-((3-(2-methyloxazol-5-yl)-1H-indazol-5-yl)amino)-2,3-dihydro-1H-indene-5-carbonitrile, enantiomer 2 (3.80 mg, 42%) as a red solid. ¹H NMR (400 MHz, MeOH-d₄) δ 7.68-7.75 (m, 2H), 7.60 (d, J=7.82 Hz, 1H), 7.43-7.48 (m, 2H), 7.34 (s, 1H), 7.21 (dd, J=8.92, 2.08 Hz, 1H), 5.57-5.64 (m, 1H), 3.49-3.71 (m, 2H), 2.60 (s, 3H). MS-ESI (m/z) calc'd for C₂₁H₁₆F₂N₅O [M+H]⁺: 392.1. Found 392.1.

### Example 156: 4-Fluoro-1-((3-(2-methyloxazol-5-yl)-1H-indazol-5-yl)oxy)-2,3-dihydro-1H-indene-5-carbonitrile, enantiomer 1 and 2

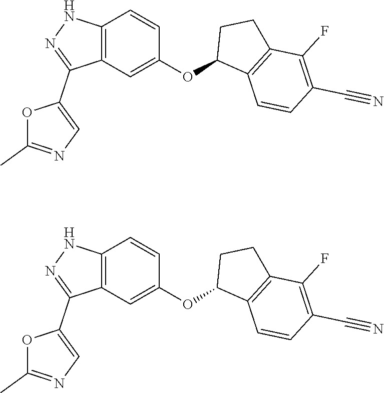 [SMILES: Cc1ncc(-c2n[nH]c3ccc(O[C@H]4CCc5c4ccc(C#N)c5F)cc23)o1 ; Cc1ncc(-c2n[nH]c3ccc(O[C@@H]4CCc5c4ccc(C#N)c5F)cc23)o1]

### Step 1: 4-Fluoro-1-((3-(2-methyloxazol-5-yl)-1-((2-(trimethylsilyl)ethoxy)methyl)-1H-indazol-5-yl)oxy)-2,3-dihydro-1H-indene-5-carbonitrile

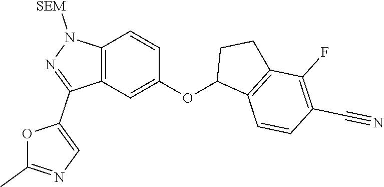 [SMILES: Cc1ncc(-c2nn(COCC[Si](C)(C)C)c3ccc(OC4CCc5c4ccc(C#N)c5F)cc23)o1]

To a mixture of 3-(2-methyloxazol-5-yl)-1-((2-(trimethylsilyl)ethoxy)methyl)-1H-indazol-5-ol (80 mg, 231.57 umol), 4-fluoro-1-hydroxy-2,3-dihydro-1H-indene-5-carbonitrile (41.03 mg, 231.57 umol) in toluene (1 mL) were added tributylphosphine (93.70 mg, 463.14 umol) and 1,1′-(azodicarbonyl)dipiperidine (116.86 mg, 463.14 umol) at 0° C. Then the mixture was degassed and purged with N₂ (3×) at 0° C., and then the mixture was stirred at 100° C. for 2 hrs under an N₂ atmosphere. The reaction mixture was combined with an identical reaction using 20 mg of 3-(2-methyloxazol-5-yl)-1-((2-(trimethylsilyl)ethoxy)methyl)-1H-indazol-5-ol. The combined mixtures were concentrated to give a residue. The residue was diluted with H₂O and extracted with EtOAc (3×). The combined organic phases were dried over anhydrous Na₂SO₄ and filtered. The filtrate was concentrated and purified by preparative-TLC (SiO₂, petroleum ether/EtOAc=1/1, Rf=0.58) to afford the title compound (105 mg, 71%) as a yellow solid. MS-ESI (m/z) calc'd for C₂₇H₃₀FN₄O₃Si [M+H]⁺: 505.2. Found 505.1.

### Step 2: 4-Fluoro-1-((1-(hydroxymethyl)-3-(2-methyloxazol-5-yl)-H-indazol-5-yl)oxy)-2,3-dihydro-1H-indene-5-carbonitrile

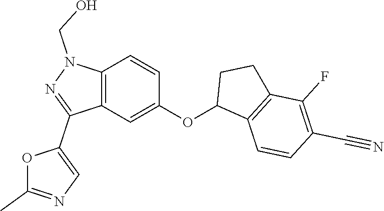 [SMILES: Cc1ncc(-c2nn(CO)c3ccc(OC4CCc5c4ccc(C#N)c5F)cc23)o1]

To a solution of 4-fluoro-1-((3-(2-methyloxazol-5-yl)-1-((2-(trimethylsilyl)ethoxy)methyl)-1H-indazol-5-yl)oxy)-2,3-dihydro-1H-indene-5-carbonitrile (110 mg, 217.98 umol) in DCM (4 mL) was added TFA (0.8 mL). The mixture was stirred at 20° C. for 2 hrs. The reaction mixture was then basified with saturated aqueous NaHCO₃ to pH=8 at 0° C. and extracted with EtOAc (3×). The combined organic phases were dried over anhydrous Na₂SO₄ and the mixture was filtered. The filtrate was concentrated under vacuum to afford the title compound (140 mg) as a yellow oil which was used without further purification. MS-ESI (m/z) calc'd for C₂₂H₁₈FN₄O₃ [M+H]⁺: 405.1. Found 405.1.

### Step 3: 4-Fluoro-1-((3-(2-methyloxazol-5-yl)-1H-indazol-5-yl)oxy)-2,3-dihydro-1H-indene-5-carbonitrile

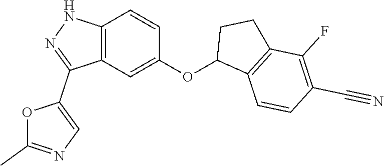 [SMILES: Cc1ncc(-c2n[nH]c3ccc(OC4CCc5c4ccc(C#N)c5F)cc23)o1]

To a solution of 4-fluoro-1-((1-(hydroxymethyl)-3-(2-methyloxazol-5-yl)-1H-indazol-5-yl)oxy)-2,3-dihydro-1H-indene-5-carbonitrile (140 mg, 346.20 umol) in dioxane (1 mL) was added NH₄OH (1 mL, 25% purity). The mixture was stirred at 20° C. for 1 hr. The reaction mixture was diluted with H₂O and extracted with EtOAc (3×). The combined organic phases were dried over anhydrous Na₂SO₄ and the mixture was filtered. The filtrate was concentrated under vacuum to give a residue that was purified by prep-TLC (SiO₂, petroleum ether/EtOAc=0/1, Rf=0.14) to afford the title compound (9 mg, 7%). MS-ESI (m/z) calc'd for C₂₁H₁₆FN₄O₂ [M+H]⁺: 375.1. Found 375.0.

### Step 4: 4-Fluoro-1-((3-(2-methyloxazol-5-yl)-1H-indazol-5-yl)oxy)-2,3-dihydro-1H-indene-5-carbonitrile, enantiomer 1 and 2

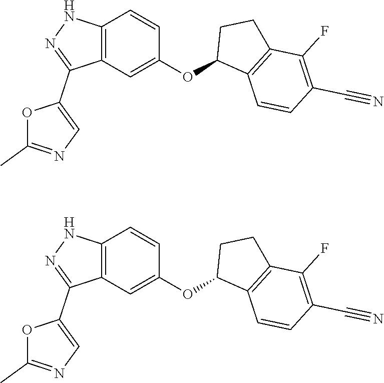 [SMILES: Cc1ncc(-c2n[nH]c3ccc(O[C@H]4CCc5c4ccc(C#N)c5F)cc23)o1 ; Cc1ncc(-c2n[nH]c3ccc(O[C@@H]4CCc5c4ccc(C#N)c5F)cc23)o1]

4-Fluoro-1-((3-(2-methyloxazol-5-yl)-1H-indazol-5-yl)oxy)-2,3-dihydro-1H-indene-5-carbonitrile was subjected to chiral separation using Method FU to afford 4-fluoro-1-((3-(2-methyloxazol-5-yl)-1H-indazol-5-yl)oxy)-2,3-dihydro-1H-indene-5-carbonitrile, enantiomer 1 (4.39 mg, 48%) as a colorless gum. ¹H NMR (400 MHz, DMSO-d₆) δ 13.33 (br s, 1H), 7.81 (br t, J=6.8 Hz, 1H), 7.70 (s, 1H), 7.59-7.51 (m, 2H), 7.45 (d, J=7.9 Hz, 1H), 7.16 (br d, J=8.8 Hz, 1H), 6.14 (br t, J=5.6 Hz, 1H), 3.21-3.11 (m, 1H), 3.06-2.95 (m, 1H), 2.81-2.69 (m, 1H), 2.53 (br s, 3H), 2.22-2.14 (m, 1H). MS-ESI (m/z) calc'd for C₂₁H₁₆FN₄O₂ [M+H]⁺: 375.1. Found 375.1. A later eluting fraction was also isolated to afford 4-fluoro-1-((3-(2-methyloxazol-5-yl)-1H-indazol-5-yl)oxy)-2,3-dihydro-1H-indene-5-carbonitrile, enantiomer 2 (2.81 mg, 31%) as a yellow solid. ¹H NMR (400 MHz, DMSO-d₆) δ 13.34 (s, 1H), 7.81 (dd, J=6.1, 7.7 Hz, 1H), 7.71 (s, 1H), 7.57-7.52 (m, 2H), 7.44 (d, J=7.8 Hz, 1H), 7.16 (dd, J=2.3, 9.0 Hz, 1H), 6.17-6.09 (m, 1H), 3.20-3.11 (m, 1H), 3.05-2.96 (m, 1H), 2.77-2.66 (m, 1H), 2.53 (s, 3H), 2.22-2.13 (m, 1H). MS-ESI (m/z) calc'd for C₂₁H₁₆FN₄O₂ [M+H]⁺: 375.1. Found 375.1.

### Example 157: 5-((3-(1H-Pyrazol-5-yl)-1H-indazol-5-yl)amino)-5,6,7,8-tetrahydronaphthalene-2-carbonitrile, enantiomer 1 and 2

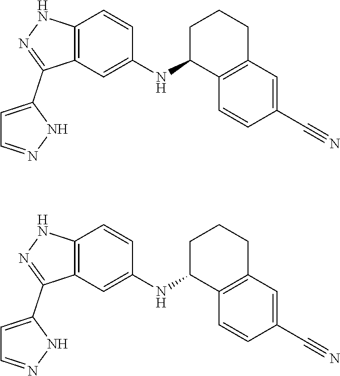 [SMILES: N#Cc1ccc2c(c1)CCC[C@@H]2Nc1ccc2[nH]nc(-c3ccn[nH]3)c2c1 ; N#Cc1ccc2c(c1)CCC[C@H]2Nc1ccc2[nH]nc(-c3ccn[nH]3)c2c1]

### Step 1: 5-((3-(1H-Pyrazol-5-yl)-1H-indazol-5-yl)amino)-5,6,7,8-tetrahydronaphthalene-2-carbonitrile

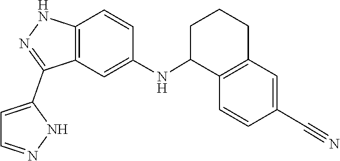 [SMILES: N#Cc1ccc2c(c1)CCCC2Nc1ccc2[nH]nc(-c3ccn[nH]3)c2c1]

To a solution of 5-((3-iodo-1H-indazol-5-yl)amino)-5,6,7,8-tetrahydronaphthalene-2-carbonitrile (200 mg, 482.81 umol) in EtOH (4 mL) and H₂O (1 mL) were added 5-(4,4,5,5-tetramethyl-1,3-dioxolan-2-yl)-1H-pyrazole (281.05 mg, 1.45 mmol), Pd(Amphos)Cl₂ (34.19 mg, 48.28 umol), and AcOK (142.15 mg, 1.45 mmol) at 20° C. The mixture was stirred at 100° C. for 3 hrs under an N₂ atmosphere. The reaction mixture was concentrated and purified by preparative-HPLC using Method FV to afford the title compound (80 mg, 47%) as a white solid. MS-ESI (m/z) calc'd for C₂₁H₁₉N₆ [M+H]⁺: 355.2. Found 355.1.

### Step 2: 5-((3-(1H-Pyrazol-5-yl)-1H-indazol-5-yl)amino)-5,6,7,8-tetrahydronaphthalene-2-carbonitrile, enantiomer 1 and 2

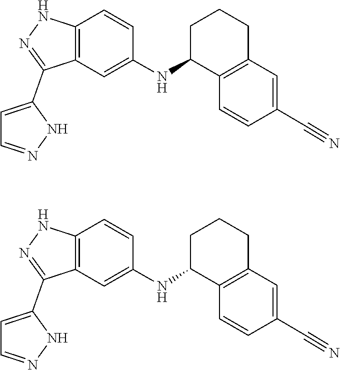 [SMILES: N#Cc1ccc2c(c1)CCC[C@@H]2Nc1ccc2[nH]nc(-c3ccn[nH]3)c2c1 ; N#Cc1ccc2c(c1)CCC[C@H]2Nc1ccc2[nH]nc(-c3ccn[nH]3)c2c1]

5-((3-(1H-Pyrazol-5-yl)-1H-indazol-5-yl)amino)-5,6,7,8-tetrahydronaphthalene-2-carbonitrile (9 mg) was subjected to chiral separation using Method FW to afford 5-((3-(1H-pyrazol-5-yl)-1H-indazol-5-yl)amino)-5,6,7,8-tetrahydronaphthalene-2-carbonitrile, enantiomer 1 (4.01 mg, 44%) as a yellow oil. ¹H NMR (400 MHz, DMSO-d₆) δ 12.48-13.44 (m, 2H), 7.61-7.86 (m, 2H), 7.50-7.59 (m, 2H), 7.20-7.48 (m, 2H), 6.96 (br d, J=8.63 Hz, 1H), 6.67 (br s, 1H), 5.82 (br d, J=6.75 Hz, 1H), 4.62 (br s, 1H), 2.72-2.93 (m, 2H), 1.70-2.04 (m, 4H). MS-ESI (m/z) calc'd for C₂₁H₁₉N₆ [M+H]⁺: 355.2. Found 355.2. A later eluting fraction was also isolated to afford 5-((3-(1H-pyrazol-5-yl)-1H-indazol-5-yl)amino)-5,6,7,8-tetrahydronaphthalene-2-carbonitrile, enantiomer 2 (4.54 mg, 50%) as a yellow oil. ¹H NMR (400 MHz, DMSO-d₆) δ 12.50-13.32 (m, 2H), 7.76 (br s, 1H), 7.63 (s, 1H), 7.51-7.59 (m, 2H), 7.18-7.46 (m, 2H), 6.96 (br d, J=8.76 Hz, 1H), 6.67 (br s, 1H), 5.82 (br d, J=7.75 Hz, 1H), 4.63 (br s, 1H), 2.74-2.91 (m, 2H), 1.78-2.01 (m, 4H). MS-ESI (m/z) calc'd for C₂₁H₁₉N₆ [M+H]⁺: 355.2. Found 355.2.

### Example 158: 8-((3-(3-Methylisothiazol-5-yl)-1H-indazol-5-yl)amino)-5,6,7,8-tetrahydroquinoline-3-carbonitrile, enantiomer 1 and 2

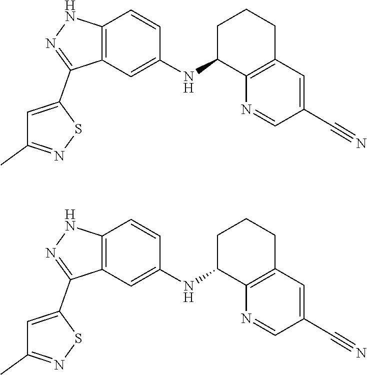 [SMILES: Cc1cc(-c2n[nH]c3ccc(N[C@H]4CCCc5cc(C#N)cnc54)cc23)sn1 ; Cc1cc(-c2n[nH]c3ccc(N[C@@H]4CCCc5cc(C#N)cnc54)cc23)sn1]

### Step 1: 8-((3-Iodo-1H-indazol-5-yl)amino)-5,6,7,8-tetrahydroquinoline-3-carbonitrile

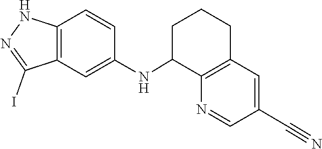 [SMILES: N#Cc1cnc2c(c1)CCCC2Nc1ccc2[nH]nc(I)c2c1]

To a solution of 3-iodo-1H-indazol-5-amine (150 mg, 579.05 umol) and 8-oxo-5,6,7,8-tetrahydroquinoline-3-carbonitrile (99.70 mg, 579.05 umol) in MeOH (3 mL) was added AcOH (3.48 mg, 57.90 umol) and the mixture was stirred at 20° C. for 0.5 hr. Then NaBH₃CN (181.94 mg, 2.90 mmol) was added and the mixture was stirred at 20° C. for an additional 11 hrs. The reaction mixture was filtered, the solid was washed with MeOH (2×) and dried under vacuum to afford the title compound (180 mg, 75%) as a yellow solid. MS-ESI (m/z) calc'd for C₁₇H₁₃IN₅ [M−H]⁻: 414.0. Found 413.9.

### Step 2: 3-Methyl-5-(tributylstannyl)isothiazole

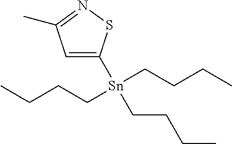 [SMILES: CCC[CH2][Sn]([CH2]CCC)([CH2]CCC)[c]1cc(C)ns1]

To a solution of 5-bromo-3-methyl-isothiazole (90 mg, 505.48 umol) in THF (4.5 mL) was added n-BuLi (2.5 M, 242.63 uL) at −78° C. and the mixture was stirred at −78° C. for 1 hr. Then tributylchlorostannane (164.53 mg, 505.48 umol) in THF (0.5 mL) was added. The resulting mixture was stirred at −78° C. for an additional 2 hrs. The reaction mixture was quenched with saturated aqueous NH₄Cl and extracted with EtOAc (3×). The combined organic layers were dried over Na₂SO₄, filtered, and concentrated to give a residue. The residue was purified by preparative-TLC (SiO₂, petroleum ether/EtOAc=20/1, Rf=0.43) to afford the title compound (127 mg, 65%) as a colorless oil. ¹H NMR (400 MHz, CDCl₃) δ 6.93 (s, 1H) 2.49 (s, 3H) 1.43-1.54 (m, 6H) 1.23-1.30 (m, 6H) 0.97-1.10 (m, 6H) 0.83 (t, J=7 Hz, 9H). MS-ESI (m/z) calc'd for C₁₆H₃₂NSSn [M+H]⁺: 390.1. Found 390.3.

### Step 3: 8-((3-(3-Methylisothiazol-5-yl)-1H-indazol-5-yl)amino)-5,6,7,8-tetrahydroquinoline-3-carbonitrile

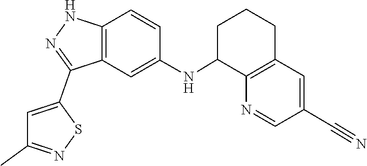 [SMILES: Cc1cc(-c2n[nH]c3ccc(NC4CCCc5cc(C#N)cnc54)cc23)sn1]

A mixture of 3-methyl-5-(tributylstannyl)isothiazole (90 mg, 231.84 umol), 8-((3-iodo-1H-indazol-5-yl)amino)-5,6,7,8-tetrahydroquinoline-3-carbonitrile (96.27 mg, 231.84 umol), and Pd(PPh₃)₂Cl₂ (16.27 mg, 23.18 umol) in DMF (4 mL) was degassed and purged with N₂ (3×) at 20° C. The mixture was then stirred at 100° C. for 2 hrs under an N₂ atmosphere. The reaction mixture was concentrated and purified by preparative-HPLC using Method FX to afford the title compound (15 mg, 17%) as a light yellow solid. MS-ESI (m/z) calc'd for C₂₁H₁₉N₆S [M+H]⁺: 387.1. Found 387.0.

### Step 4: 8-((3-(3-Methylisothiazol-5-yl)-1H-indazol-5-yl)amino)-5,6,7,8-tetrahydroquinoline-3-carbonitrile, enantiomer 1 and 2

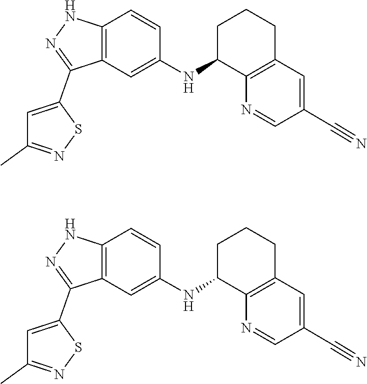 [SMILES: Cc1cc(-c2n[nH]c3ccc(N[C@H]4CCCc5cc(C#N)cnc54)cc23)sn1 ; Cc1cc(-c2n[nH]c3ccc(N[C@@H]4CCCc5cc(C#N)cnc54)cc23)sn1]

8-((3-(3-Methylisothiazol-5-yl)-1H-indazol-5-yl)amino)-5,6,7,8-tetrahydroquinoline-3-carbonitrile was subjected to chiral separation using Method FW to afford 8-((3-(3-methylisothiazol-5-yl)-1H-indazol-5-yl)amino)-5,6,7,8-tetrahydroquinoline-3-carbonitrile, enantiomer 1 (4.27 mg, 47%) as a yellow solid. ¹H NMR (400 MHz, DMSO-d₆) δ 13.17 (br d, J=20 Hz, 1H) 8.82 (d, J=2 Hz, 1H) 8.15 (d, J=2 Hz, 1H) 7.68 (s, 1H) 7.38 (d, J=9 Hz, 1H) 7.13 (s, 1H) 7.01 (dd, J=9, 2 Hz, 1H) 6.02 (d, J=8 Hz, 1H) 4.79-4.90 (m, 1H) 2.77-2.97 (m, 2H) 1.79-2.08 (m, 4H). MS-ESI (m/z) calc'd for C₂₁H₁₉N₆S [M+H]⁺: 387.1. Found 387.1. A later eluting fraction was also isolated to afford 8-((3-(3-methylisothiazol-5-yl)-1H-indazol-5-yl)amino)-5,6,7,8-tetrahydroquinoline-3-carbonitrile, enantiomer 2 (4.14 mg, 44%) as a yellow solid. ¹H NMR (400 MHz, DMSO-d₆) δ 8.82 (d, J=2 Hz, 1H) 8.15 (d, J=2 Hz, 1H) 7.67 (s, 1H) 7.39 (d, J=9 Hz, 1H) 7.12 (s, 1H) 7.00 (dd, J=9, 2 Hz, 1H) 6.01 (d, J=8 Hz, 1H) 4.81-4.90 (m, 1H) 2.79-2.96 (m, 2H) 2.00-2.07 (m, 2H) 1.80-1.98 (m, 2H). MS-ESI (m/z) calc'd for C₂₁H₁₉N₆S [M+H]⁺: 387.1. Found 387.1.

### Example 159: 2-Methyl-8-((3-(2-methyloxazol-5-yl)-1H-indazol-5-yl)amino)-5,6,7,8-tetrahydroquinoline-3-carbonitrile, enantiomer 1 and 2

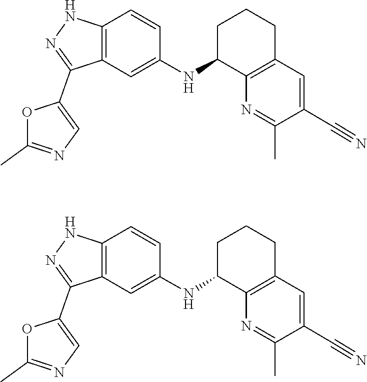 [SMILES: Cc1ncc(-c2n[nH]c3ccc(N[C@H]4CCCc5cc(C#N)c(C)nc54)cc23)o1 ; Cc1ncc(-c2n[nH]c3ccc(N[C@@H]4CCCc5cc(C#N)c(C)nc54)cc23)o1]

### Step 1: 2-Chloro-3-cyano-5,6,7,8-tetrahydroquinoline 1-oxide

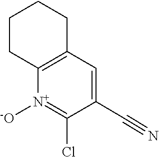

To a solution of 2-chloro-5,6,7,8-tetrahydroquinoline-3-carbonitrile (2 g, 10.38 mmol) in DCM (20 mL) was added m-CPBA (4.48 g, 20.76 mmol) at 20° C. The mixture was then stirred at 50° C. for 24 hrs. The reaction mixture was quenched by addition of 10% aqueous Na₂SO₃ at 0° C., and then extracted with EtOAc (3×). The combined organic layers were dried over Na₂SO₄, filtered and the filtrate was evaporated to dryness to afford the title compound (2 mg, 92%) as a yellow solid. ¹H NMR (400 MHz, DMSO-d₆) δ 7.80 (s, 1H), 2.79 (td, J=6.28, 16.29 Hz, 4H), 1.65-1.87 (m, 4H). MS-ESI (m/z) calc'd for C₁₀H₁₀ClN₂O [M+H]⁺: 209.0/211.0. Found 209.2/211.2.

### Step 2: 2-Chloro-8-hydroxy-5,6,7,8-tetrahydroquinoline-3-carbonitrile

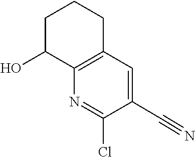 [SMILES: N#Cc1cc2c(nc1Cl)C(O)CCC2]

A solution of 2-chloro-3-cyano-5,6,7,8-tetrahydroquinoline 1-oxide (1.8 g, 8.63 mmol) in TFAA (18 mL) was stirred at 20° C. for 2 hrs. The reaction mixture was concentrated to give a residue that was basified with 2 M NaOH aqueous solution to pH=8 and extracted with EtOAc (3×). The combined organic phases were dried over anhydrous Na₂SO₄ and filtered. The filtrate was concentrated and purified by flash silica gel chromatography (ISCO; 20 g SepaFlash column) using a 0-50% EtOAc/petroleum ether gradient eluent to afford the title compound (500 mg, 28%) as a yellow solid. ¹H NMR (400 MHz, DMSO-d₆) δ 8.27 (s, 1H), 5.67 (br s, 1H), 4.56 (t, J=4.22 Hz, 1H), 2.76-2.88 (m, 1H), 2.64-2.75 (m, 1H), 1.81-1.95 (m, 3H), 1.66-1.78 (m, 1H). MS-ESI (m/z) calc'd for C₁₀H₁₀ClN₂O [M+H]⁺: 209.0/211.0. Found 209.0/211.0.

### Step 3: 2-Chloro-8-oxo-5,6,7,8-tetrahydroquinoline-3-carbonitrile

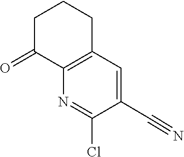 [SMILES: N#Cc1cc2c(nc1Cl)C(=O)CCC2]

To a solution of 2-chloro-8-hydroxy-5,6,7,8-tetrahydroquinoline-3-carbonitrile (150 mg, 718.93 umol) in DCM (1 mL) was added Dess-Martin periodinane (365.91 mg, 862.71 umol). The mixture was stirred at 20° C. for 1 hr. The reaction mixture was adjusted to pH=8 with saturated aqueous Na₂CO₃ and extracted with EtOAc (3×). The combined organic layers were dried over Na₂SO₄ and filtered. The filtrate was concentrated and purified by preparative-TLC (SiO₂, petroleum ether/EtOAc=1/1, Rf=0.3) to afford the title compound (50 mg, 34%) as a yellow solid. ¹H NMR (400 MHz, CDCl₃) δ 8.07 (s, 1H), 3.11 (t, J=6.11 Hz, 2H), 2.82-2.94 (m, 2H), 2.24-2.35 (m, 2H). MS-ESI (m/z) calc'd for C₁₀H₈ClN₂O [M+H]⁺: 207.0/209.0. Found 207.2/209.2.

### Step 4: 2-Methyl-8-oxo-5,6,7,8-tetrahydroquinoline-3-carbonitrile

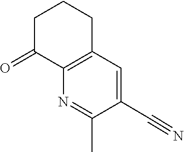

To a solution of 2-chloro-8-oxo-5,6,7,8-tetrahydroquinoline-3-carbonitrile (60 mg, 290.38 umol) in dioxane (3 mL) were added 2,4,6-trimethyl-1,3,5,2,4,6-trioxatriborinane (43.74 mg, 348.45 umol), XPhos-Pd-G2 (22.85 mg, 29.04 umol) and K₃PO₄ (123.27 mg, 580.75 umol) at 20° C. The mixture was then stirred at 60° C. for 12 hrs under an N₂ atmosphere. The reaction mixture was concentrated and purified by preparative-TLC (100% EtOAc, Rf=0.65) to afford the title compound (40 mg, 74%) as an orange solid. ¹H NMR (400 MHz, CDCl₃) δ 7.92 (s, 1H), 3.04 (t, J=6.00 Hz, 2H), 2.82-2.88 (m, 5H), 2.18-2.29 (m, 2H). MS-ESI (m/z) calc'd for C₁₁H₁₁N₂O [M+H]⁺: 187.1. Found 187.3.

### Step 5: 2-Methyl-8-((3-(2-methyloxazol-5-yl)-1H-indazol-5-yl)amino)-5,6,7,8-tetrahydroquinoline-3-carbonitrile

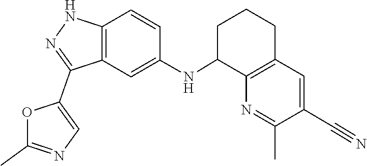 [SMILES: Cc1ncc(-c2n[nH]c3ccc(NC4CCCc5cc(C#N)c(C)nc54)cc23)o1]

To a solution of 2-methyl-8-oxo-5,6,7,8-tetrahydroquinoline-3-carbonitrile (50 mg, 268.51 umol) and 3-(2-methyloxazol-5-yl)-1H-indazol-5-amine (57.52 mg, 268.51 umol) in MeOH (5 mL) was added AcOH (32.25 mg, 537.03 umol). The mixture was stirred at 20° C. for 2 hrs. Then NaBH₃CN (50.62 mg, 805.54 umol) was added and the mixture was stirred at 20° C. for 2 hrs. The reaction mixture was concentrated and purified by preparative-HPLC using Method FY to afford the title compound (9 mg, 9%) as a yellow solid. MS-ESI (m/z) calc'd for C₂₂H₂₁N₆O [M−H]⁻: 383.2. Found 383.2.

### Step 6: 2-Methyl-8-((3-(2-methyloxazol-5-yl)-1H-indazol-5-yl)amino)-5,6,7,8-tetrahydroquinoline-3-carbonitrile, enantiomer 1 and 2

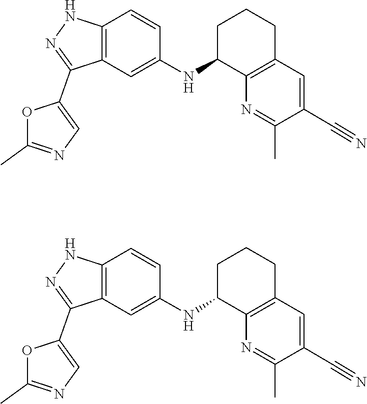 [SMILES: Cc1ncc(-c2n[nH]c3ccc(N[C@H]4CCCc5cc(C#N)c(C)nc54)cc23)o1 ; Cc1ncc(-c2n[nH]c3ccc(N[C@@H]4CCCc5cc(C#N)c(C)nc54)cc23)o1]

2-Methyl-8-((3-(2-methyloxazol-5-yl)-1H-indazol-5-yl)amino)-5,6,7,8-tetrahydroquinoline-3-carbonitrile was subjected to chiral separation using Method FZ to afford 2-methyl-8-((3-(2-methyloxazol-5-yl)-1H-indazol-5-yl)amino)-5,6,7,8-tetrahydroquinoline-3-carbonitrile, enantiomer 1 (2.91 mg, 32%) as a yellow solid. ¹H NMR (400 MHz, DMSO-d₆) δ 12.99 (br s, 1H), 8.06 (s, 1H), 7.50 (s, 1H), 7.35 (br d, J=8.66 Hz, 1H), 7.08 (s, 1H), 6.99 (br d, J=8.91 Hz, 1H), 5.88 (d, J=7.03 Hz, 1H), 4.68-4.73 (m, 1H), 2.82-2.90 (m, 1H), 2.71-2.81 (m, 1H), 2.60 (s, 3H), 1.87-2.04 (m, 3H), 1.74-1.83 (m, 1H). MS-ESI (m/z) calc'd for C₂₂H₂₁N₆O [M+H]⁺: 385.2. Found 385.2. A later eluting fraction was also isolated to afford 2-methyl-8-((3-(2-methyloxazol-5-yl)-1H-indazol-5-yl)amino)-5,6,7,8-tetrahydroquinoline-3-carbonitrile, enantiomer 2 (2.84 mg, 31%) as a yellow solid. ¹H NMR (400 MHz, DMSO-d₆) δ 13.00 (s, 1H) 8.06 (s, 1H) 7.50 (s, 1H) 7.34 (d, J=8.78 Hz, 1H) 7.08 (s, 1H) 6.99 (d, J=9.03 Hz, 1H) 5.89 (d, J=7.15 Hz, 1H) 4.71 (d, J=5.77 Hz, 1H) 2.82-2.90 (m, 1H) 2.71-2.80 (m, 1H) 2.60 (s, 3H) 1.87-2.05 (m, 3H) 1.73-1.83 (m, 1H). MS-ESI (m/z) calc'd for C₂₂H₂₁N₆O [M+H]⁺: 385.2. Found 385.2.

### Example 160: 6-Fluoro-1-((3-(2-methyloxazol-5-yl)-1H-indazol-5-yl)oxy)-2,3-dihydro-1H-indene-5-carbonitrile, enantiomer 1 and 2

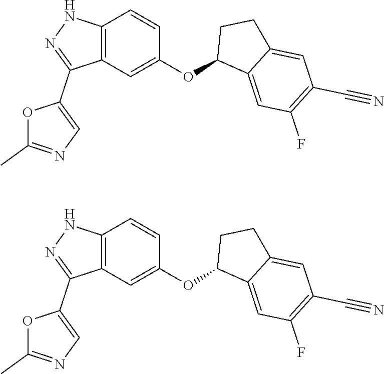 [SMILES: Cc1ncc(-c2n[nH]c3ccc(O[C@H]4CCc5cc(C#N)c(F)cc54)cc23)o1 ; Cc1ncc(-c2n[nH]c3ccc(O[C@@H]4CCc5cc(C#N)c(F)cc54)cc23)o1]

### Step 1: 5-Bromo-3-iodo-1-((2-(trimethylsilyl)ethoxy)methyl)-1H-indazole

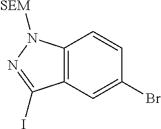 [SMILES: C[Si](C)(C)CCOCn1nc(I)c2cc(Br)ccc21]

To a solution of 5-bromo-3-iodo-1H-indazole (5 g, 15.48 mmol) in THF (50 mL) was added N-cyclohexyl-N-methyl-cyclohexanamine (4.84 g, 24.78 mmol) at 0° C. The mixture was stirred at 0° C. for 0.5 hr. Then SEM-Cl (3.36 g, 20.12 mmol) was added and the mixture was stirred at 50° C. for 5 hrs. The mixture was diluted with H₂O and extracted with EtOAc (3×). The combined organic phases were dried over anhydrous Na₂SO₄, filtered, and the filtrate was concentrated and purified by flash silica gel chromatography (ISCO; 40 g SepaFlash column) using a 0-5% EtOAc/petroleum ether gradient eluent to afford the title compound (4 g, 57%) as a yellow oil. ¹H NMR (400 MHz, CDCl₃) δ 7.61-7.68 (m, 1H), 7.52-7.60 (m, 1H), 7.36-7.47 (m, 1H), 5.68-5.82 (m, 2H), 3.52-3.67 (m, 2H), 0.84-0.96 (m, 2H), −0.08-0.05 (m, 9H).

### Step 2: 5-Bromo-3-(I-ethoxyvinyl)-1-((2-(trimethylsilyl)ethoxy)methyl)-1H-indazole

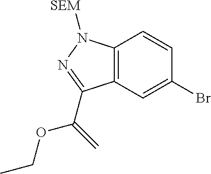 [SMILES: C=C(OCC)c1nn(COCC[Si](C)(C)C)c2ccc(Br)cc12]

A mixture of 5-bromo-3-iodo-1-((2-(trimethylsilyl)ethoxy)methyl)-1H-indazole (8 g, 17.64 mmol), tributyl(1-ethoxyvinyl)stannane (7.64 g, 21.2 mmol), Pd(PPh₃)₄ (2.04 g, 1.76 mmol) in toluene (80 mL) was degassed and purged with N₂ (3×) at 25° C. Then the mixture was stirred at 100° C. for 12 hrs under an N₂ atmosphere. The mixture was concentrated and purified by flash silica gel chromatography (ISCO; 80 g SepaFlash column) using a 0-7% EtOAc/petroleum ether gradient eluent to afford the title compound (5.2 g, 74%) a yellow oil. ¹H NMR (400 MHz, CDCl₃) δ 7.96-8.06 (m, 1H), 7.60-7.66 (m, 1H), 7.37-7.46 (m, 1H), 5.84-5.91 (m, 2H), 4.81-4.85 (m, 1H), 4.71 (d, J=2.88 Hz, 1H), 4.17 (q, J=7.13 Hz, 1H), 4.04-4.10 (m, 1H), 3.66-3.72 (m, 2H), 1.26-1.36 (m, 3H), 0.96-0.99 (m, 2H), 0.00-0.01 (m, 9H). MS-ESI (m/z) calc'd for C₁₇H₂₆BrN₂O₂Si [M+H]⁺: 397.1/399.1. Found: 397.2/399.2.

### Step 3: 5-Bromo-3-(I-ethoxyvinyl)-1-((2-(trimethylsilyl)ethoxy)methyl)-1H-indazole

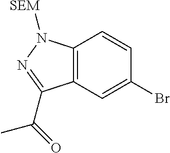 [SMILES: CC(=O)c1nn(COCC[Si](C)(C)C)c2ccc(Br)cc12]

To a solution of 5-bromo-3-(1-ethoxyvinyl)-1-((2-(trimethylsilyl)ethoxy)methyl)-1H-indazole (4.2 g, 10.56 mmol) in THF (62 mL) was added HCl (2 M, 13 mL) at 20° C. The mixture was stirred at 20° C. for 1 hr. The reaction mixture was diluted with H₂O and extracted with EtOAc (3×). The combined organic phases were dried over anhydrous Na₂SO₄ and filtered. The filtrate was concentrated and purified by flash silica gel chromatography (ISCO; 12 g SepaFlash column) using a 0-5% EtOAc/petroleum ether gradient eluent to afford the title compound (780 mg, 20%) as a yellow solid. ¹H NMR (400 MHz, CDCl₃) δ 8.55 (d, J=1.63 Hz, 1H), 7.53-7.57 (m, 1H), 7.48-7.51 (m, 1H), 5.76 (s, 2H), 3.54-3.60 (m, 2H), 2.67-2.74 (m, 3H), 0.86-0.93 (m, 2H), 0.10-0.03 (m, 9H).

### Step 4: 5-(5-Bromo-1-((2-(trimethylsilyl)ethoxy)methyl)-1H-indazol-3-yl)-2-methyloxazole

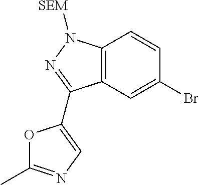 [SMILES: Cc1ncc(-c2nn(COCC[Si](C)(C)C)c3ccc(Br)cc23)o1]

To a solution of 5-bromo-3-(1-ethoxyvinyl)-1-((2-(trimethylsilyl)ethoxy)methyl)-1H-indazole (720 mg, 1.95 mmol) in DMSO (11 mL) was added 12 (989.60 mg, 3.90 mmol) at 25° C. The mixture was then stirred at 110° C. for 45 minutes. Then 2-aminopropanoic acid (347.37 mg, 3.90 mmol) was added and the mixture was stirred at 110° C. for an additional 15 minutes. The reaction mixture was diluted with H₂O and extracted with EtOAc (3×). The combined organic phases were dried over anhydrous Na₂SO₄ and filtered. The filtrate was concentrated to give a residue that was purified by flash silica gel chromatography (ISCO; 4 g SepaFlash column) using a 0-6% EtOAc/petroleum ether gradient eluent and further purified by preparative-HPLC using Method GA to afford the title compound (180 mg, 22%) as a yellow solid. MS-ESI (m/z) calc'd for C₁₇H₂₃BrN₃O₂Si [M+H]⁺:408.1/410.1. Found: 407.9/409.9.

### Step 5: 2-Methyl-5-(5-(4,4,5,5-tetramethyl-1,3,2-dioxaborolan-2-yl)-1-((2-(trimethylsilyl)ethoxy)methyl)-1H-indazol-3-yl)oxazole

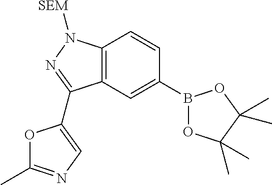

A mixture of 5-(5-bromo-1-((2-(trimethylsilyl)ethoxy)methyl)-1H-indazol-3-yl)-2-methyloxazole (180 mg, 440.78 umol), bis(pinacolato)diboron (223.86 mg, 881.57 umol), Pd(dppf)Cl₂ (32.25 mg, 44.08 umol), and KOAc (129.78 mg, 1.32 mmol) in dioxane (3 mL) was degassed and purged with N₂ (3×) at 20° C. The mixture was then stirred at 120° C. for 12 hrs. The mixture was filtered and the filtrate was concentrated to give a residue that was purified by flash silica gel chromatography (ISCO; 4 g SepaFlash column) using a 0-8% EtOAc/petroleum ether gradient eluent to afford the title compound (180 mg, 90%) as a yellow oil. MS-ESI (m/z) calc'd for C₂₃H₃₅BN₃O₄Si [M+H]⁺:456.2. Found: 456.4.

### Step 6: 3-(2-Methyloxazol-5-yl)-1-((2-(trimethylsilyl)ethoxy)methyl)-1H-indazol-5-ol

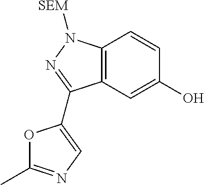 [SMILES: Cc1ncc(-c2nn(COCC[Si](C)(C)C)c3ccc(O)cc23)o1]

To a solution of 2-methyl-5-(5-(4,4,5,5-tetramethyl-1,3,2-dioxaborolan-2-yl)-1-((2-(trimethylsilyl)ethoxy)methyl)-1H-indazol-3-yl)oxazole (180 mg, 395.23 umol) in THF (2 mL) were added sodium perborate tetrahydrate (182.43 mg, 1.19 mmol) and H₂O (2 mL) at 20° C., The mixture was stirred at 50° C. for 1 hr. The reaction mixture was diluted with H₂O and extracted with EtOAc (3×). The combined organic phases were dried over anhydrous Na₂SO₄, filtered, and the filtrate was concentrated to afford the title compound (100 mg, 73%) as a white solid. ¹H NMR (400 MHz, CDCl₃) δ 7.51-7.61 (m, 2H), 7.31 (s, 1H), 7.17 (dd, J=8.94, 1.56 Hz, 1H), 5.77 (s, 2H), 3.55-3.65 (m, 2H), 2.68 (s, 3H), 0.89 (d, J=8.00 Hz, 2H), −0.09-0.02 (m, 9H). MS-ESI (m/z) calc'd for C₁₇H₂₄N₃O₃Si [M+H]⁺:346.2. Found: 346.3.

### Step 7: 6-Fluoro-1-((3-(2-methyloxazol-5-yl)-1-((2-(trimethylsilyl)ethoxy)methyl)-1H-indazol-5-yl)oxy)-2,3-dihydro-1H-indene-5-carbonitrile

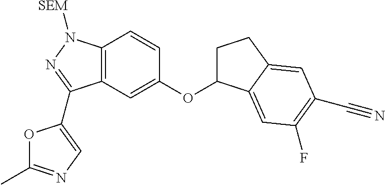 [SMILES: Cc1ncc(-c2nn(COCC[Si](C)(C)C)c3ccc(OC4CCc5cc(C#N)c(F)cc54)cc23)o1]

To a solution of 3-(2-methyloxazol-5-yl)-1-((2-(trimethylsilyl)ethoxy)methyl)-1H-indazol-5-ol (58.49 mg, 169.32 umol) and 6-fluoro-1-hydroxy-2,3-dihydro-1H-indene-5-carbonitrile (30 mg, 169.32 umol) in toluene (3 mL) were added tributylphosphine (34.26 mg, 169.32 umol) and ADDP (42.72 mg, 169.32 umol) at 0° C. The mixture was degassed and purged with N₂ (3×), and then the mixture was stirred at 100° C. for 12 hrs under an N₂ atmosphere. The reaction mixture was filtered; the filtrate was concentrated and purified by preparative-TLC (100% EtOAc, Rf=0.67) to afford the title compound (38 mg, 44%) as yellow solid. MS-ESI (m/z) calc'd for C₂₇H₃₀N₄O₃FSi [M+H]⁺:505.2. Found: 505.3.

### Step 8: 6-Fluoro-1-((1-(hydroxymethyl)-3-(2-methyloxazol-5-yl)-H-indazol-5-yl)oxy)-2,3-dihydro-1H-indene-5-carbonitrile

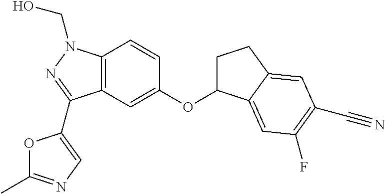 [SMILES: Cc1ncc(-c2nn(CO)c3ccc(OC4CCc5cc(C#N)c(F)cc54)cc23)o1]

To a solution of 6-fluoro-1-((3-(2-methyloxazol-5-yl)-1-((2-(trimethylsilyl)ethoxy)methyl)-1H-indazol-5-yl)oxy)-2,3-dihydro-1H-indene-5-carbonitrile (38 mg, 75.30 umol) in CH₂Cl₂ (1 mL) was added TFA (0.2 mL) at 20° C. The mixture was stirred at 20° C. for 2 hrs. The reaction mixture was basified by addition saturated aqueous NaHCO₃ at 0° C. to pH=7, and then diluted with H₂O and extracted with EtOAc (3×). The combined organic phases were dried over anhydrous Na₂SO₄, filtered, and the filtrate was concentrated to afford the title compound (30 mg, 98%) as yellow oil, which was used directly without further purification. MS-ESI (m/z) calc'd for C₂₂H₁₈FN₄O₃ [M+H]⁺: 405.1. Found 405.3.

### Step 9: 6-Fluoro-1-((3-(2-methyloxazol-5-yl)-1H-indazol-5-yl)oxy)-2,3-dihydro-1H-indene-5-carbonitrile

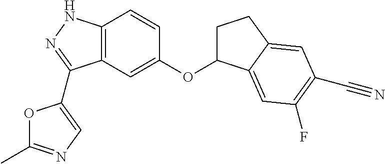 [SMILES: Cc1ncc(-c2n[nH]c3ccc(OC4CCc5cc(C#N)c(F)cc54)cc23)o1]

To a solution of 6-fluoro-1-((1-(hydroxymethyl)-3-(2-methyloxazol-5-yl)-1H-indazol-5-yl)oxy)-2,3-dihydro-1H-indene-5-carbonitrile (30 mg, 74.19 umol) in dioxane (1 mL) was added NH₄OH (910.00 mg, 6.49 mmol) at 20° C. The mixture was stirred at 20° C. for 0.5 hr. The reaction mixture was diluted with H₂O and extracted with EtOAc (3×). The combined organic phases were dried over anhydrous Na₂SO₄, filtered, the filtrate was concentrated and purified by preparative-HPLC using Method GB to afford the title compound (5 mg, 18%) as a yellow oil. MS-ESI (m/z) calc'd for C₂₁H₁₆FN₄O₂ [M+H]⁺: 375.1. Found: 375.0.

### Step 10: 6-Fluoro-1-((3-(2-methyloxazol-5-yl)-1H-indazol-5-yl)oxy)-2,3-dihydro-1H-indene-5-carbonitrile, enantiomer 1 and 2

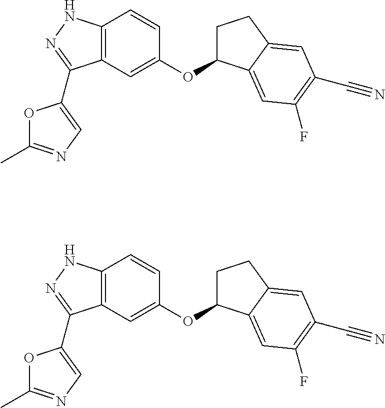 [SMILES: Cc1ncc(-c2n[nH]c3ccc(O[C@H]4CCc5cc(C#N)c(F)cc54)cc23)o1 ; Cc1ncc(-c2n[nH]c3ccc(O[C@H]4CCc5cc(C#N)c(F)cc54)cc23)o1]

Fluoro-1-((3-(2-methyloxazol-5-yl)-1H-indazol-5-yl)oxy)-2,3-dihydro-1H-indene-5-carbonitrile was subjected to chiral separation using Method GC to afford 6-fluoro-1-((3-(2-methyloxazol-5-yl)-1H-indazol-5-yl)oxy)-2,3-dihydro-1H-indene-5-carbonitrile, enantiomer 1 (0.71 mg, 14%) as a white solid. ¹H NMR (400 MHz, DMSO-d₆) δ 13.33 (s, 1H), 7.91 (d, J=6.00 Hz, 1H), 7.70 (s, 1H), 7.50-7.60 (m, 3H), 7.17 (dd, J=9.13, 2.13 Hz, 1H), 6.06 (t, J=5.88 Hz, 1H), 3.04 (br d, J=4.88 Hz, 1H), 2.94 (br d, J=7.75 Hz, 1H), 2.53 (br s, 4H), 2.10-2.17 (m, 1H). MS-ESI (m/z) calc'd for C₂₁H₁₆FN₄O₂ [M+H]⁺: 375.1. Found 375.1. A later eluting fraction was also isolated to afford 6-fluoro-1-((3-(2-methyloxazol-5-yl)-1H-indazol-5-yl)oxy)-2,3-dihydro-1H-indene-5-carbonitrile, enantiomer 2 (0.73 mg, 15%) as a white solid. ¹H NMR (400 MHz, DMSO-d₆) δ 13.39 (s, 1H), 7.98 (d, J=5.88 Hz, 1H), 7.77 (s, 1H), 7.55-7.67 (m, 3H), 7.24 (dd, J=8.94, 2.19 Hz, 1H), 6.13 (t, J=5.82 Hz, 1H), 3.13 (br dd, J=12.95, 7.69 Hz, 1H), 2.95-3.03 (m, 1H), 2.75-2.81 (m, 1H), 2.60 (s, 3H), 2.17-2.23 (m, 1H). MS-ESI (m/z) calc'd for C₂₁H₁₆FN₄O₂ [M+H]⁺: 375.1. Found 375.1.

### Example 161: 8-((3-(Pyrazin-2-yl)-1H-indazol-5-yl)oxy)-5,6,7,8-tetrahydroquinoline-3-carbonitrile, enantiomer 1 and 2

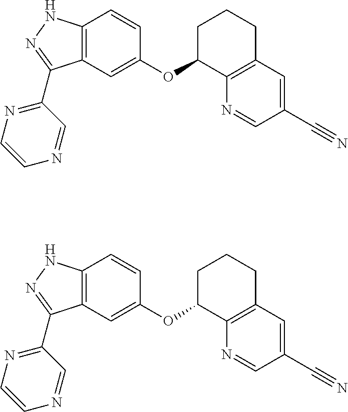 [SMILES: N#Cc1cnc2c(c1)CCC[C@@H]2Oc1ccc2[nH]nc(-c3cnccn3)c2c1 ; N#Cc1cnc2c(c1)CCC[C@H]2Oc1ccc2[nH]nc(-c3cnccn3)c2c1]

### Step 1: 5-((tert-Butyldimethylsilyl)oxy)-1H-indazole

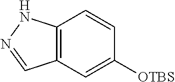 [SMILES: CC(C)(C)[Si](C)(C)Oc1ccc2[nH]ncc2c1]

To a solution of 1H-indazol-5-ol (1 g, 7.46 mmol) and imidazole (1.52 g, 22.37 mmol) in DMF (10 mL) was add tert-butyldimethylsilyl chloride (6.74 g, 44.73 mmol) at 20° C. The mixture was then stirred at 100° C. for 12 hrs. The reaction was combined with another 300 mg scale reaction before work up. The combined reaction mixture was concentrated and purified by flash silica gel column chromatography (ISCO; 20 g SepaFlash column) using a 0-13% EtOAc/petroleum ether gradient eluent to afford the title compound (1.8 g, 76%) as a pale red solid. ¹H NMR (400 MHz, CDCl₃) δ 9.97 (br s, 1H), 8.01 (d, J=0.88 Hz, 1H), 7.35-7.41 (m, 1H), 7.15 (dd, J=0.66, 2.19 Hz, 1H), 6.97-7.03 (m, 1H), 1.02 (s, 9H), 0.18-0.27 (m, 6H). MS-ESI (m/z) calc'd for C₁₃H₂₁N₂₀Si [M+H]⁺: 249.1. Found 249.2.

### Step 2: 3-Iodo-1H-indazol-5-ol

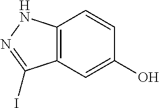 [SMILES: Oc1ccc2[nH]nc(I)c2c1]

To a solution of 5-((tert-butyldimethylsilyl)oxy)-1H-indazole (1 g, 4.03 mmol) in DMF (45 mL) was added NIS (1.81 g, 8.05 mmol) and the mixture was stirred at 25° C. for 12 hrs. The reaction mixture was concentrated to give a residue that was purified by flash silica gel column chromatography (ISCO; 20 g SepaFlash column) using a 0-80% EtOAc/petroleum ether gradient eluent to afford the title compound (670 mg, 64%). ¹H NMR (400 MHz, DMSO-d₆) δ 13.22 (br s, 1H), 9.35 (br s, 1H), 7.38 (d, J=8.88 Hz, 1H), 6.97 (dd, J=2.19, 8.94 Hz, 1H), 6.64 (d, J=2.00 Hz, 1H). MS-ESI (m/z) calc'd for C₇H₆₁N₂O [M+H]⁺: 260.9. Found 261.1.

### Step 3: 8-((3-Iodo-1H-indazol-5-yl)oxy)-5,6,7,8-tetrahydroquinoline-3-carbonitrile

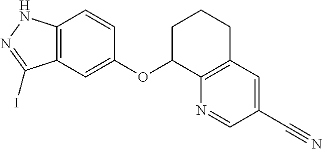 [SMILES: N#Cc1cnc2c(c1)CCCC2Oc1ccc2[nH]nc(I)c2c1]

To a solution of 3-iodo-1H-indazol-5-ol (300 mg, 1.15 mmol) and 8-hydroxy-5,6,7,8-tetrahydroquinoline-3-carbonitrile (200.97 mg, 1.15 mmol) in toluene (30 mL) were added ADDP (582.18 mg, 2.31 mmol) and tributylphosphine (466.84 mg, 2.31 mmol) at 0° C. The mixture was then stirred at 90° C. for 2 hrs. The mixture was concentrated to give a residue that was diluted with H₂O and extracted with EtOAc (3×). The combined organic phases were dried over anhydrous Na₂SO₄, filtered, and the filtrate was concentrated and purified by preparative-HPLC using Method GD to afford the title compound (70 mg, 14%) as a yellow solid. ¹H NMR (400 MHz, DMSO-d₆) δ 13.41 (s, 1H), 8.88 (d, J=1.96 Hz, 1H), 8.22 (s, 1H), 7.50 (d, J=9.05 Hz, 1H), 7.18 (dd, J=2.32, 9.05 Hz, 1H), 7.09 (d, J=2.08 Hz, 1H), 5.56 (t, J=3.67 Hz, 1H), 2.01 (s, 2H), 1.80-1.94 (m, 2H), 1.19 (t, J=7.09 Hz, 2H). MS-ESI (m/z) calc'd for C₁₇H₁₄IN₄O [M+H]⁺: 417.0. Found 416.9.

### Step 4: 8-((3-(Pyrazin-2-yl)-1H-indazol-5-yl)oxy)-5,6,7,8-tetrahydroquinoline-3-carbonitrile

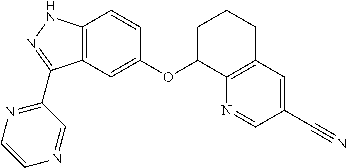 [SMILES: N#Cc1cnc2c(c1)CCCC2Oc1ccc2[nH]nc(-c3cnccn3)c2c1]

A mixture of 8-((3-iodo-1H-indazol-5-yl)oxy)-5,6,7,8-tetrahydroquinoline-3-carbonitrile (50 mg), 2-(tributylstannyl)pyrazine (44.34 mg, 120.13 umol), and Pd(PPh₃)₂Cl₂ (8.43 mg, 12.01 umol) in DMF (5 mL) was degassed and purged with N₂ (3×) at 20° C. The mixture was then stirred at 120° C. for 12 hrs under an N₂ atmosphere. The reaction mixture was concentrated and purified by preparative-HPLC using Method GE to afford the title compound (11 mg, 25%) as a yellow solid. MS-ESI (m/z) calc'd for C₂₁H₁₇N₆O [M+H]⁺: 369.1. Found 369.1.

### Step 5: 8-((3-(Pyrazin-2-yl)-1H-indazol-5-yl)oxy)-5,6,7,8-tetrahydroquinoline-3-carbonitrile, enantiomer 1 and 2

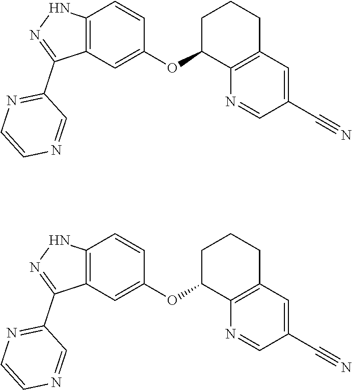 [SMILES: N#Cc1cnc2c(c1)CCC[C@@H]2Oc1ccc2[nH]nc(-c3cnccn3)c2c1 ; N#Cc1cnc2c(c1)CCC[C@H]2Oc1ccc2[nH]nc(-c3cnccn3)c2c1]

8-((3-(Pyrazin-2-yl)-1H-indazol-5-yl)oxy)-5,6,7,8-tetrahydroquinoline-3-carbonitrile was subjected to chiral separation using Method GF to afford 8-((3-(pyrazin-2-yl)-1H-indazol-5-yl)oxy)-5,6,7,8-tetrahydroquinoline-3-carbonitrile, enantiomer 1 (2.01 mg, 22%) as a yellow gum. ¹H NMR (400 MHz, DMSO-d₆) δ 13.58 (br s, 1H), 9.38 (d, J=1.34 Hz, 1H), 8.88 (d, J=1.83 Hz, 1H), 8.77 (dd, J=1.59, 2.45 Hz, 1H), 8.59 (d, J=2.57 Hz, 1H), 8.23 (d, J=1.71 Hz, 1H), 8.12 (d, J=2.20 Hz, 1H), 7.60 (d, J=8.92 Hz, 1H), 7.26 (dd, J=2.26, 8.99 Hz, 1H), 5.48-5.53 (m, 1H), 2.93-3.03 (m, 1H), 2.73-2.89 (m, 1H), 2.24-2.32 (m, 1H), 1.93-2.08 (m, 2H), 1.79-1.91 (m, 1H). MS-ESI (m/z) calc'd for C₂₁H₁₇N₆O [M+H]⁺: 369.1. Found 369.2. A later eluting fraction was also isolated to afford 8-((3-(pyrazin-2-yl)-1H-indazol-5-yl)oxy)-5,6,7,8-tetrahydroquinoline-3-carbonitrile, enantiomer 2 (2.53 mg, 28%) as a yellow gum. ¹H NMR (400 MHz, DMSO-d₆) δ 13.58 (br s, 1H), 9.39 (d, J=1.47 Hz, 1H), 8.89 (d, J=1.71 Hz, 1H), 8.72-8.80 (m, 1H), 8.59 (d, J=2.57 Hz, 1H), 8.24 (s, 1H), 8.10-8.16 (m, 1H), 7.61 (d, J=8.92 Hz, 1H), 7.27 (dd, J=2.32, 9.05 Hz, 1H), 5.48-5.55 (m, 1H), 2.95-3.04 (m, 1H), 2.76-2.88 (m, 1H), 2.29 (br d, J=12.23 Hz, 1H), 1.95-2.09 (m, 2H), 1.87 (br s, 1H). MS-ESI (m/z) calc'd for C₂₁H₁₇N₆O [M+H]⁺: 369.1. Found 369.1.

### Example 162: 8-Deuterio-8-((3-(oxazol-5-yl)-1H-indazol-5-yl)amino)-5,6,7,8-tetrahydroquinoline-3-carbonitrile enantiomer 1 and 2

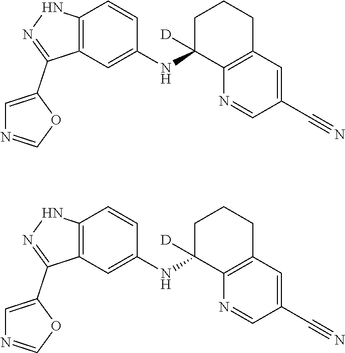 [SMILES: [2H][C@]1(Nc2ccc3[nH]nc(-c4cnco4)c3c2)CCCc2cc(C#N)cnc21 ; [2H][C@@]1(Nc2ccc3[nH]nc(-c4cnco4)c3c2)CCCc2cc(C#N)cnc21]

### Step 1: 8-Deuterio-8-((3-(oxazol-5-yl)-1H-indazol-5-yl)amino)-5,6,7,8-tetrahydroquinoline-3-carbonitrile

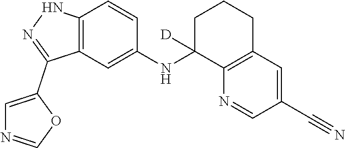 [SMILES: [2H]C1(Nc2ccc3[nH]nc(-c4cnco4)c3c2)CCCc2cc(C#N)cnc21]

To a solution of 8-oxo-5,6,7,8-tetrahydroquinoline-3-carbonitrile (8.60 mg, 49.95 umol) and 3-(oxazol-5-yl)-1H-indazol-5-amine (10 mg, 49.95 umol) in MeOH (1 mL) was added AcOH (299.97 ug, 5.00 umol). The mixture was stirred at 20° C. for 2 hrs. Then sodium borodeuteride (11.34 mg, 299.71 umol) was added. The mixture was stirred at 20° C. for 2 hrs. This procedure was repeat 7 additional times and the reaction mixtures were combined. The final combined mixture was concentrated and purified by preparative-HPLC using Method GH to afford the title compound (10 mg, 7%) as a white solid. MS-ESI (m/z) calc'd for C₂₀H₁₆DN₆O [M+H]⁺: 358.1. Found 358.0.

### Step 2: 8-Deuterio-8-((3-(oxazol-5-yl)-1H-indazol-5-yl)amino)-5,6,7,8-tetrahydroquinoline-3-carbonitrile, enantiomer 1 and 2

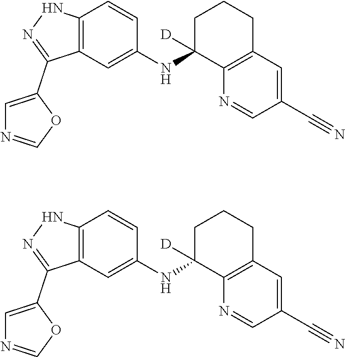 [SMILES: [2H][C@]1(Nc2ccc3[nH]nc(-c4cnco4)c3c2)CCCc2cc(C#N)cnc21 ; [2H][C@@]1(Nc2ccc3[nH]nc(-c4cnco4)c3c2)CCCc2cc(C#N)cnc21]

8-Deuterio-8-((3-(oxazol-5-yl)-1H-indazol-5-yl)amino)-5,6,7,8-tetrahydroquinoline-3-carbonitrile was subjected to chiral separation using Method GI to afford: 8-deuterio-8-((3-(oxazol-5-yl)-1H-indazol-5-yl)amino)-5,6,7,8-tetrahydroquinoline-3-carbonitrile, enantiomer 1 (1.37 mg, 13%) as a yellow solid. ¹H NMR (400 MHz, DMSO-d₆) δ ppm 13.11 (br s, 1H), 8.81 (d, J=2.0 Hz, 1H), 8.47 (s, 1H), 8.14 (d, J=2.0 Hz, 1H), 7.66 (s, 1H), 7.36 (d, J=8.9 Hz, 1H), 7.09 (d, J=1.8 Hz, 1H), 7.00 (dd, J=2.1, 8.9 Hz, 1H), 5.94 (s, 1H), 2.95-2.78 (m, 2H), 2.06-1.99 (m, 2H), 1.98-1.90 (m, 1H), 1.88-1.80 (m, 1H). MS-ESI (m/z) calc'd for C₂₀H₁₆DN₆O [M+H]⁺: 358.1. Found 358.1. A later eluting fraction was also isolated to afford 8-deuterio-8-((3-(oxazol-5-yl)-1H-indazol-5-yl)amino)-5,6,7,8-tetrahydroquinoline-3-carbonitrile, enantiomer 2 (1.48 mg, 14%) as a yellow solid. ¹H NMR (400 MHz, DMSO-d₆) δ ppm 13.09 (br s, 1H), 8.81 (d, J=1.9 Hz, 1H), 8.48 (s, 1H), 8.14 (d, J=1.9 Hz, 1H), 7.66 (s, 1H), 7.36 (d, J=8.9 Hz, 1H), 7.09 (s, 1H), 7.00 (dd, J=2.0, 9.0 Hz, 1H), 5.94 (s, 1H), 2.97-2.78 (m, 2H), 2.05-1.98 (m, 2H), 1.98-1.90 (m, 1H), 1.89-1.80 (m, 1H). MS-ESI (m/z) calc'd for C₂₀H₁₆DN₆O [M+H]⁺: 358.1. Found 358.1.

### Example 163: 5-[[3-(1,3-Oxazol-5-yl)-1H-indazol-5-yl]oxy]-5,6,7,8-tetrahydroquinoxaline-2-carbonitrile, enantiomer 1 and 2

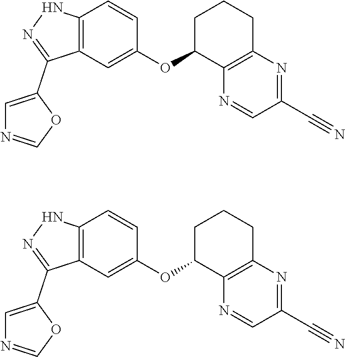 [SMILES: N#Cc1cnc2c(n1)CCC[C@@H]2Oc1ccc2[nH]nc(-c3cnco3)c2c1 ; N#Cc1cnc2c(n1)CCC[C@H]2Oc1ccc2[nH]nc(-c3cnco3)c2c1]

### Step 1: 5,6,7,8-Tetrahydroquinoxalin-2(1H)-one

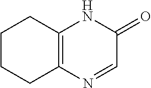 [SMILES: O=c1cnc2c([nH]1)CCCC2]

A suspension of glycinamide hydrochloride (3.32 g, 30 mmol) in MeOH (12 mL) was cooled to −30° C., then an ice-cold solution of 1,2-cyclohexanedione (3.36 g, 30 mmol) in MeOH (12 mL) was added. While stirring, 12.5 M sodium hydroxide (6.0 mL, 75 mmol) was added slowly to keep the internal temperature below −20° C. The mixture was gradually warmed to 25° C., then 37% HCl (4 mL) was added followed by NaHCO₃ (1.1 g). The mixture was stirred for 5 minutes then filtered under vacuum, washed with H₂O and dried to afford the title compound (3.1 g, 69%) as a yellow solid. ¹H NMR (400 MHz, DMSO-d₆) δ 11.94 (s, 1H), 7.76 (d, J=1.1 Hz, 1H), 2.59-2.49 (m, 4H), 1.79-1.61 (m, 4H). MS-ESI (m/z) calc'd for C₈H₁₁N₂O [M+H]⁺: 151.2. Found 150.9.

### Step 2: 2-Chloro-5,6,7,8-tetrahydroquinoxaline

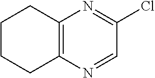 [SMILES: Clc1cnc2c(n1)CCCC2]

A suspension of 5,6,7,8-tetrahydroquinoxalin-2(1H)-one (3.12 g, 20.75 mmol) and tetraethylammonium chloride (3.44 g, 20.75 mmol) in phosphorus(V) oxychloride (30.0 mL, 320.88 mmol) was heated at 100° C. for 24 hrs. The solvent was evaporated; the residue was taken up in K₂CO₃(aq) and extracted with DCM (3×). The combined organic layers were passed through a phase separator and evaporated to obtain a residue which was purified by silica gel column chromatography using a 0-25% EtOAc/cyclohexane gradient eluent to afford the title compound (2.45 g, 70%) as a yellow oil. ¹H NMR (400 MHz, DMSO-d₆) δ 8.48 (s, 1H), 2.86 (ddt, J=6.7, 5.0, 2.5 Hz, 4H), 1.84 (p, J=3.2 Hz, 4H). MS-ESI (m/z) calc'd for C₈H₁₀ClN₂ [M+H]⁺: 169.2 Found 168.9.

### Step 3: 3-Chloro-5,6,7,8-tetrahydroquinoxaline 1-oxide

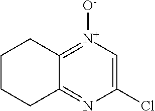 [SMILES: [O-][n+]1cc(Cl)nc2c1CCCC2]

To a solution of 2-chloro-5,6,7,8-tetrahydroquinoxaline (2.45 g, 14.53 mmol) in DCE (50 mL) was added MCPBA (4.3 g, 17.44 mmol) and the mixture was stirred at 65° C. for 15 hrs. After cooling, the mixture was diluted with DCM and washed with saturated aqueous K₂CO₃ (×3). The organic phase was passed through a phase separator and evaporated to afford the title compound (2.54 g, 95%) as a clear oil. ¹H NMR (400 MHz, DMSO-d₆) δ 8.55 (s, 1H), 2.80 (t, J=5.8 Hz, 2H), 2.67 (t, J=6.0 Hz, 2H), 1.90-1.68 (m, 4H). MS-ESI (m/z) calc'd for C₈H₁₀ClN[N⁺][O⁻] [M+H]⁺: 185.0 Found 185.0.

### Step 4: 2-Chloro-5,6,7,8-tetrahydroquinoxalin-5-ol

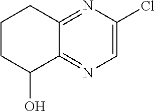 [SMILES: OC1CCCc2nc(Cl)cnc21]

To a solution of 3-chloro-5,6,7,8-tetrahydroquinoxaline 1-oxide (200.0 mg, 0.79 mmol) in MeOH (4 mL) was added sodium borohydride (60.03 mg, 1.59 mmol) and the mixture was stirred at r.t. for 1 hr and then concentrated under reduced pressure. The residue was taken up in EtOAc and water. The organic phase was separated, dried over Na₂SO₄, filtered, and concentrated under reduced pressure to give a residue. The residue was purified by silica gel column chromatography using a 0-50% EtOAc/cyclohexane gradient eluent to afford the title compound (172 mg, 87%), as a colorless oil. ¹H NMR (400 MHz, DMSO-d₆) δ 7.14 (d, J=7.8 Hz, 1H), 5.73 (d, J=5.9 Hz, 1H), 5.05 (q, J=6.4 Hz, 1H), 2.95 (ddd, J=16.3, 8.8, 3.9 Hz, 1H), 2.81-2.69 (m, 1H), 2.46-2.35 (m, 1H), 1.90-1.76 (m, 1H). MS-ESI (m/z) calc'd for C₈H₁₀ClN₂O [M+H]⁺: 185.0 Found [M+H−H₂O]⁺: 166.9.

### Step 5: 5-(5-((2-Chloro-5,6,7,8-tetrahydroquinoxalin-5-yl)oxy)-1-((2-(trimethylsilyl)ethoxy)methyl)-1H-indazol-3-yl)oxazole

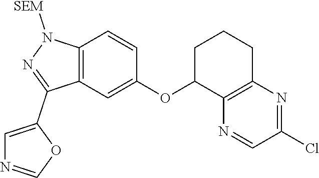 [SMILES: C[Si](C)(C)CCOCn1nc(-c2cnco2)c2cc(OC3CCCc4nc(Cl)cnc43)ccc21]

To a solution of 2-chloro-5,6,7,8-tetrahydroquinoxalin-5-ol (153.85 mg, 0.50 mmol), 3-(oxazol-5-yl)-1-((2-(trimethylsilyl)ethoxy)methyl)-1H-indazol-5-ol (165.72 mg, 0.50 mmol) and triphenylphosphine (262.29 mg, 1 mmol) was added DEAD (157.46 uL, 1 mmol) and the mixture was stirred at 25° C. for 2 hrs. The solvent was evaporated and the residue was purified by silica gel column chromatography using a 0-30% EtOAc/cyclohexane gradient eluent to afford the title compound (178 mg, 71%) as a glassy oil. ¹H NMR (400 MHz, DMSO-d₆) δ 8.64 (s, 1H), 8.55 (d, J=5.5 Hz, 3H), 7.91 (s, 1H), 7.75 (d, J=9.0 Hz, 1H), 5.78 (d, J=4.7 Hz, 2H), 4.24-3.84 (m, 1H), 3.56 (t, J=7.9 Hz, 2H), 3.16-2.65 (m, 2H), 2.36-1.73 (m, 4H), 0.82 (t, J=8.0 Hz, 2H), 0.10-−0.36 (m, 9H). MS-ESI (m/z) calc'd for C₂₄H₂₉ClN₅O₃Si [M+H]⁺: 498.2 Found 498.2.

### Step 6: 5-((3-(Oxazol-5-yl)-1-((2-(trimethylsilyl)ethoxy)methyl)-1H-indazol-5-yl)oxy)-5,6,7,8-tetrahydroquinoxaline-2-carbonitrile

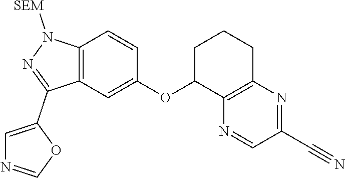 [SMILES: C[Si](C)(C)CCOCn1nc(-c2cnco2)c2cc(OC3CCCc4nc(C#N)cnc43)ccc21]

5-(5-((2-Chloro-5,6,7,8-tetrahydroquinoxalin-5-yl)oxy)-1-((2-(trimethylsilyl)ethoxy)methyl)-1H-indazol-3-yl)oxazole (489.0 mg, 0.98 mmol), 0.1 M potassium ferrocyanide (9.82 mL, 0.98 mmol) and KOAc (96.36 mg, 0.98 mmol) were mixed together in 1,4-dioxane (10 mL) and the mixture was degassed with N₂ for 10 minutes. XPhos (70.21 mg, 0.15 mmol) and XPhos Pd G3 (124.66 mg, 0.15 mmol) were added and the mixture was stirred at 100° C. for 1.5 hrs. The mixture was diluted with H₂O and extracted with DCM (3×). The combined organic layers were passed through a phase separator and evaporated to obtain a brown oil which was purified by silica gel column chromatography using a 0-50% EtOAc/cyclohexane gradient eluent to afford the title compound (197 mg, 41%). ¹H NMR (400 MHz, DMSO-d₆) δ 9.06 (s, 1H), 8.54 (s, 1H), 7.91 (s, 1H), 7.76 (d, J=9.1 Hz, 1H), 7.71 (d, J=2.2 Hz, 1H), 7.29 (dd, J=9.1, 2.3 Hz, 1H), 5.79 (s, 2H), 4.03 (q, J=7.2 Hz, 1H), 3.56 (t, J=8.0 Hz, 2H), 3.23-2.81 (m, 2H), 2.31-1.80 (m, 2H), 1.26-1.05 (m, 2H), 0.82 (t, J=7.9 Hz, 2H), −0.10 (s, 9H). MS-ESI (m/z) calc'd for C₂₅H₂₉N₆O₃Si [M+H]⁺: 489.2. Found 489.3.

### Step 7: 5-[[3-(1,3-Oxazol-5-yl)-1H-indazol-5-yl]oxy]-5,6,7,8-tetrahydroquinoxaline-2-carbonitrile, enantiomer 1 and 2

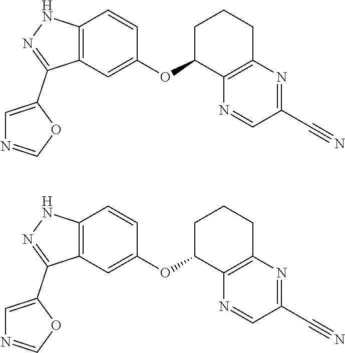 [SMILES: N#Cc1cnc2c(n1)CCC[C@@H]2Oc1ccc2[nH]nc(-c3cnco3)c2c1 ; N#Cc1cnc2c(n1)CCC[C@H]2Oc1ccc2[nH]nc(-c3cnco3)c2c1]

Prepared as described for 1-methoxy-5-((3-(oxazol-5-yl)-1H-indazol-5-yl)amino)-5,6,7,8-tetrahydronaphthalene-2-carbonitrile using 5-[3-(1,3-oxazol-5-yl)-1-(2-trimethylsilylethoxymethyl)indazol-5-yl]oxy-5,6,7,8-tetrahydroquinoxaline-2-carbonitrile in place of 1-methoxy-5-((3-(oxazol-5-yl)-1-((2-(trimethylsilyl)ethoxy)methyl)-1H-indazol-5-yl)amino)-5,6,7,8-tetrahydronaphthalene-2-carbonitrile, to afford 5-[[3-(1,3-oxazol-5-yl)-1H-indazol-5-yl]oxy]-5,6,7,8-tetrahydroquinoxaline-2-carbonitrile (13 mg, 15%), which was subjected to chiral separation using Method GJ to afford 5-[[3-(1,3-oxazol-5-yl)-1H-indazol-5-yl]oxy]-5,6,7,8-tetrahydroquinoxaline-2-carbonitrile, enantiomer 1 (1.7 mg, 2%) as a white solid. ¹H NMR (400 MHz, MeOD) δ 8.86 (d, J=1.0 Hz, 1H), 8.36 (s, 1H), 7.75 (d, J=2.3 Hz, 1H), 7.67 (s, 1H), 7.52 (dd, J=9.1, 0.7 Hz, 1H), 7.25 (dd, J=9.1, 2.3 Hz, 1H), 5.65 (t, J=4.1 Hz, 1H), 3.18 (dt, J=18.3, 5.3 Hz, 1H), 3.03 (ddd, J=18.4, 9.5, 5.8 Hz, 1H), 2.48-2.36 (m, 1H), 2.34-2.11 (m, 2H), 2.09-1.97 (m, 1H). MS-ESI (m/z) calc'd for C₁₉H₁₅N₆O₂ [M+H]⁺: 359.1. Found 359.2. A later eluting fraction was also isolated to afford 5-[[3-(1,3-oxazol-5-yl)-1H-indazol-5-yl]oxy]-5,6,7,8-tetrahydroquinoxaline-2-carbonitrile, enantiomer 2 (1.4 mg, 2%) as a white solid. ¹H NMR (400 MHz, MeOD) δ 8.86 (d, J=1.0 Hz, 1H), 8.36 (s, 1H), 7.75 (d, J=2.3 Hz, 1H), 7.67 (s, 1H), 7.52 (dd, J=9.0, 0.7 Hz, 1H), 7.25 (dd, J=9.0, 2.3 Hz, 1H), 5.65 (t, J=4.2 Hz, 1H), 3.18 (dt, J=18.1, 4.6 Hz, 1H), 3.03 (ddd, J=18.1, 9.2, 5.7 Hz, 1H), 2.49-2.36 (m, 1H), 2.34-2.11 (m, 2H), 2.03 (tt, J=7.3, 4.8 Hz, 1H). MS-ESI (m/z) calc'd for C₁₉H₁₅N₆O₂ [M+H]⁺: 359.1. Found 359.2.

### Example 164: 2,4-Dimethyl-8-[[3-(1,3-oxazol-5-yl)-1H-indazol-5-yl]amino]-5,6,7,8-tetrahydroquinoline-3-carbonitrile, enantiomer 1 and 2

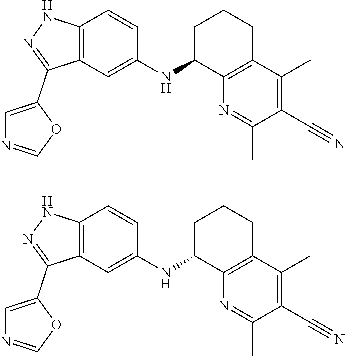 [SMILES: Cc1nc2c(c(C)c1C#N)CCC[C@@H]2Nc1ccc2[nH]nc(-c3cnco3)c2c1 ; Cc1nc2c(c(C)c1C#N)CCC[C@H]2Nc1ccc2[nH]nc(-c3cnco3)c2c1]

### Step 1: 2-Chloro-3-cyano-4-methyl-5,6,7,8-tetrahydroquinoline 1-oxide

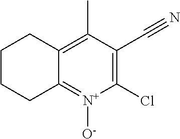

To a solution of 2-chloro-4-methyl-5,6,7,8-tetrahydroquinoline-3-carbonitrile (1.39 g, 6.73 mmol) in trifluoroacetic acid (25 mL) was added a 30 wt. % solution of hydrogen peroxide in H₂O (2.06 mL, 20.18 mmol) and the mixture was stirred at 75° C. for 16 hrs. A further 1.0 mL of H₂O₂ (30 wt. % in H₂O) was added and the mixture was stirred at 75° C. for an additional 24 hrs. Water was added and the solution was neutralized by addition of solid K₂CO₃ and then extracted with DCM (3×). The combined organic layers were washed with H₂O (1×), passed through a phase separator and evaporated under reduced pressure to afford the title compound (1.19 g, 79%) as a light yellow solid. ¹H NMR (400 MHz, DMSO-d₆) δ 2.78-2.89 (m, 2H) 2.63-2.74 (m, 2H) 2.41 (s, 3H) 1.67-1.84 (m, 4H). MS-ESI (m/z) calc'd for C₁₁H₁₂ClN₂O [M+H]⁺: 223.1. Found 223.0.

### Step 2: 2-Chloro-8-hydroxy-4-methyl-5,6,7,8-tetrahydroquinoline-3-carbonitrile

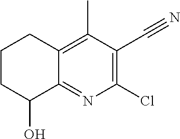

To a solution of 2-chloro-3-cyano-4-methyl-5,6,7,8-tetrahydroquinoline 1-oxide (800.0 mg, 3.59 mmol) in DCM (20 mL) was added trifluoroacetic anhydride (1.5 mL, 10.78 mmol) dropwise and the mixture was stirred at 25° C. for 16 hrs. The solvent was evaporated and the residue was taken up in MeOH; then K₂CO₃ was added till basic pH and the suspension was stirred at 25° C. for 1 hr. The solvent was evaporated and the residue was taken up in H₂O and extracted with DCM (3×). The combined organic layers were passed through a phase separator and evaporated to dryness. The material was purified by silica gel column chromatography using a 0-100% EtOAc/cyclohexane gradient eluent to afford the title compound (350 mg, 44%) as a white solid. ¹H NMR (400 MHz, DMSO-d₆) δ 5.59 (d, J=5.06 Hz, 1H) 4.55 (q, J=4.70 Hz, 1H) 2.68-2.78 (m, 1H) 2.53-2.63 (m, 1H) 2.45 (s, 3H) 1.69-2.00 (m, 4H). MS-ESI (m/z) calc'd for C₁₁H₁₂ClN₂O [M+H]⁺: 223.1; Found 223.0.

### Step 3: 8-Hydroxy-2,4-dimethyl-5,6,7,8-tetrahydroquinoline-3-carbonitrile

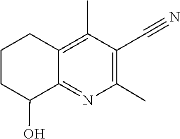

A solution of 2-chloro-8-hydroxy-4-methyl-5,6,7,8-tetrahydroquinoline-3-carbonitrile (120.0 mg, 0.54 mmol), K₂CO₃ (148.97 mg, 1.08 mmol) and 2,4,6-trimethyl-1,3,5,2,4,6-trioxatriborinane (135.3 mg, 1.08 mmol) in 1,4-dioxane (6 mL) and H₂O (3 mL) was degassed with N₂ for 15 min. Then Pd(PPh₃)₄ (124.55 mg, 0.11 mmol) was added and the mixture was heated to 100° C. for 30 minutes using microwave irradiation. Heating was then continued for an additional 30 min. The reaction mixture was partitioned between H₂O and DCM and the phases were separated. The aqueous layer was extracted with DCM (2×) and the combined organic phases were washed with H₂O (1×), passed through a phase separator and evaporated to dryness. The material was purified by silica gel column chromatography using a 0-50% EtOAc/cyclohexane gradient eluent to afford the title compound (105 mg, 96%) as an off-white solid. ¹H NMR (400 MHz, DMSO-d₆) δ 5.27 (d, J=4.40 Hz, 1H) 4.54 (q, J=4.62 Hz, 1H) 2.66-2.77 (m, 1H) 2.63 (s, 3H) 2.55-2.61 (m, 1H) 2.39 (s, 3H) 1.89-1.99 (m, 1H) 1.83 (dt, J=8.64, 4.15 Hz, 2H) 1.68-1.78 (m, 1H). MS-ESI (m/z) calc'd for C₁₂H₁₅N₂O [M+H]⁺: 203.1. Found 203.1.

### Step 4: N-(3-Cyano-2,4-dimethyl-5,6,7,8-tetrahydroquinolin-8-yl)-2-nitro-N-(3-(oxazol-5-yl)-1-((2-(trimethylsilyl)ethoxy)methyl)-1H-indazol-5-yl)benzenesulfonamide

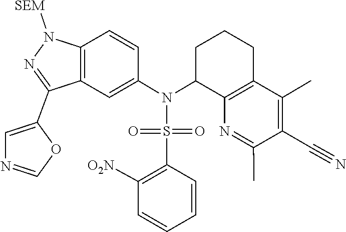 [SMILES: Cc1nc2c(c(C)c1C#N)CCCC2N(c1ccc2c(c1)c(-c1cnco1)nn2COCC[Si](C)(C)C)S(=O)(=O)c1ccccc1[N+](=O)[O-]]

To a solution of 8-hydroxy-2,4-dimethyl-5,6,7,8-tetrahydroquinoline-3-carbonitrile (105.0 mg, 0.52 mmol), 2-nitro-N-[3-(1,3-oxazol-5-yl)-1-(2-trimethylsilylethoxymethyl)indazol-5-yl]benzenesulfonamide (267.68 mg, 0.52 mmol) and triphenylphosphine (272.34 mg, 1.04 mmol) in THF (10 mL) was added diethyl azodicarboxylate (163.49 uL, 1.04 mmol) dropwise and the mixture was stirred at r.t. for 1.5 hrs. An additional 2 eq. of DEAD and 2 eq. of PPh₃ were then added and the mixture was stirred for an additional 2 hrs. The reaction mixture was partitioned between H₂O and EtOAc and the phases were separated. The aqueous layer was extracted with EtOAc (3×) and the combined organic phases were washed with brine (1×), dried over anhydrous Na₂SO₄, and evaporated to dryness. The material was purified by silica gel column chromatography using a 0-50% EtOAc/cyclohexane gradient eluent to afford the title compound (585 mg), as a beige solid. MS-ESI (m/z) calc'd for C₃₄H₃₈N₇O₆SSi [M+H]⁺: 700.2; Found 700.4.

### Step 5: 2,4-Dimethyl-8-((3-(oxazol-5-yl)-1-((2-(trimethylsilyl)ethoxy)methyl)-1H-indazol-5-yl)amino)-5,6,7,8-tetrahydroquinoline-3-carbonitrile

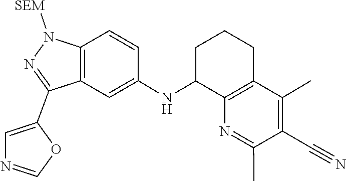 [SMILES: Cc1nc2c(c(C)c1C#N)CCCC2Nc1ccc2c(c1)c(-c1cnco1)nn2COCC[Si](C)(C)C]

To a solution of N-(3-cyano-2,4-dimethyl-5,6,7,8-tetrahydroquinolin-8-yl)-2-nitro-N-(3-(oxazol-5-yl)-1-((2-(trimethylsilyl)ethoxy)methyl)-1H-indazol-5-yl)benzenesulfonamide (363.22 mg, 0.52 mmol) in DMF (5 mL) were added K₂CO₃ (286.92 mg, 2.08 mmol) and benzenethiol (0.16 mL, 1.56 mmol) and the mixture was stirred at r.t. for 1 hr. The reaction mixture was partitioned between H₂O and EtOAc, the phases were separated, the aqueous layer was extracted with EtOAc (2×), and the combined organic phases were washed with brine (1×), dried over anhydrous Na₂SO₄, and evaporated to dryness. The residue was purified by SCX, using a 5 g cartridge, washing with MeOH and then eluting the compound with a 2 M solution of NH₃ in MeOH to afford the title compound (175 mg, 65%) as a yellow solid. ¹H NMR (400 MHz, DMSO-d₆) δ 8.51 (s, 1H) 7.73 (s, 1H) 7.55 (d, J=9.24 Hz, 1H) 7.15 (d, J=1.54 Hz, 1H) 7.07 (dd, J=9.13, 1.87 Hz, 1H) 5.99 (d, J=7.04 Hz, 1H) 5.71 (s, 2H) 4.67-4.74 (m, 1H) 3.54 (t, J=7.92 Hz, 2H) 2.73-2.82 (m, 1H) 2.62-2.69 (m, 1H) 2.58 (s, 3H) 2.43 (s, 3H) 1.77-2.06 (m, 4H) 0.81 (t, J=7.92 Hz, 2H) −0.10 (s, 9H). MS-ESI (m/z) calc'd for C₂₈H₃₅N₆O₂Si [M+H]⁺: 515.3; Found 515.4.

### Step 6: 2,4-Dimethyl-8-[[3-(1,3-oxazol-5-yl)-1H-indazol-5-yl]amino]-5,6,7,8-tetrahydroquinoline-3-carbonitrile, enantiomer 1 and 2

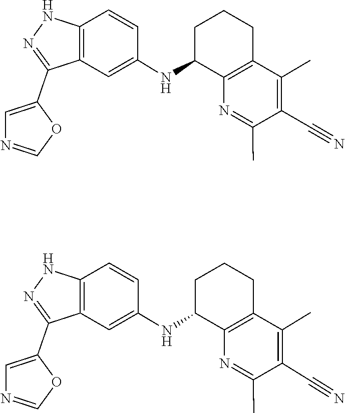 [SMILES: Cc1nc2c(c(C)c1C#N)CCC[C@@H]2Nc1ccc2[nH]nc(-c3cnco3)c2c1 ; Cc1nc2c(c(C)c1C#N)CCC[C@H]2Nc1ccc2[nH]nc(-c3cnco3)c2c1]

Prepared as described for 1-methoxy-5-((3-(oxazol-5-yl)-1H-indazol-5-yl)amino)-5,6,7,8-tetrahydronaphthalene-2-carbonitrile using 2,4-dimethyl-8-[[3-(1,3-oxazol-5-yl)-1-(2-trimethylsilylethoxymethyl)indazol-5-yl]amino]-5,6,7,8-tetrahydroquinoline-3-carbonitrile in place of 1-methoxy-5-((3-(oxazol-5-yl)-1-((2-(trimethylsilyl)ethoxy)methyl)-1H-indazol-5-yl)amino)-5,6,7,8-tetrahydronaphthalene-2-carbonitrile, to afford 2,4-dimethyl-8-[[3-(1,3-oxazol-5-yl)-1H-indazol-5-yl]amino]-5,6,7,8-tetrahydroquinoline-3-carbonitrile (87 mg, 67%), which was subjected to chiral separation using Method GK to afford 2,4-dimethyl-8-[[3-(1,3-oxazol-5-yl)-1H-indazol-5-yl]amino]-5,6,7,8-tetrahydroquinoline-3-carbonitrile, enantiomer 1 (35 mg, 27%) as an off-white solid. ¹H NMR (400 MHz, DMSO-d₆) δ 13.07 (br. s., 1H) 8.47 (s, 1H) 7.62-7.67 (m, 1H) 7.36 (d, J=8.80 Hz, 1H) 7.12 (s, 1H) 7.00 (dd, J=9.02, 1.98 Hz, 1H) 5.88 (d, J=7.04 Hz, 1H) 4.68 (br. s., 1H) 2.63-2.83 (m, 2H) 2.59 (s, 3H) 2.43 (s, 3H) 1.80-2.05 (m, 4H), MS-ESI (m/z) calc'd for C₂₂H₂₁N₆O [M+H]⁺: 385.2. Found 385.2. A later eluting fraction was also isolated to afford 2,4-dimethyl-8-[[3-(1,3-oxazol-5-yl)-1H-indazol-5-yl]amino]-5,6,7,8-tetrahydroquinoline-3-carbonitrile, enantiomer 2 (36 mg, 27%) as an off-white solid. ¹H NMR (400 MHz, DMSO-d₆) δ 12.98-13.14 (m, 1H) 8.47 (s, 1H) 7.65 (s, 1H) 7.32-7.40 (m, 1H) 7.12 (d, J=1.54 Hz, 1H) 7.00 (dd, J=8.91, 2.09 Hz, 1H) 5.88 (d, J=6.82 Hz, 1H) 4.67 (d, J=6.16 Hz, 1H) 2.62-2.82 (m, 2H) 2.59 (s, 3H) 2.43 (s, 3H) 1.85-2.05 (m, 4H). MS-ESI (m/z) calc'd for C₂₂H₂₁N₆O [M+H]⁺: 385.2. Found 385.2.

### Example 165: 2-Methoxy-4-methyl-8-[[3-(1,3-oxazol-5-yl)-1H-indazol-5-yl]amino]-5,6,7,8-tetrahydroquinoline-3-carbonitrile, enantiomer 1 and 2

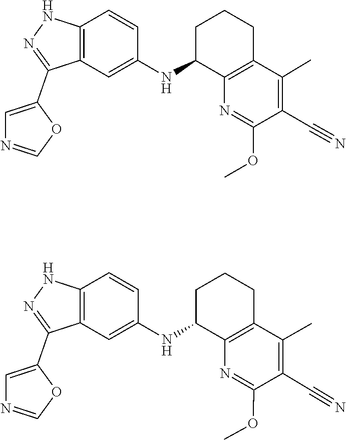 [SMILES: COc1nc2c(c(C)c1C#N)CCC[C@@H]2Nc1ccc2[nH]nc(-c3cnco3)c2c1 ; COc1nc2c(c(C)c1C#N)CCC[C@H]2Nc1ccc2[nH]nc(-c3cnco3)c2c1]

### Step 1: 4-Methyl-2-oxo-1,2,5,6,7,8-hexahydroquinoline-3-carbonitrile

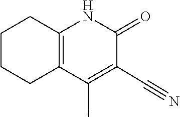

To a solution of 2-cyanoacetic acid ethyl ester (2.12 mL, 20 mmol), acetaldehyde (1.12 mL, 20 mmol) and cyclohexanone (2.07 mL, 20 mmol) in DMSO (3 mL) was added pyrrolidine (0.17 mL, 2 mmol) and the mixture was stirred for 1 hr. Ammonium acetate (2.31 g, 30 mmol) was then added and the mixture was stirred vigorously for 30 min. Additional pyrrolidine (2.01 mL, 24 mmol) was added and the reaction mixture was then stirred at 80° C. overnight. Three additional, identical reactions were performed and combined. The combined reaction mixture was partitioned between H₂O and DCM, the phases were separated, the aqueous layer was extracted with DCM (2×) and the combined organic phases were washed with H₂O (1×), passed through a phase separator and evaporated to dryness. The residue was purified by reversed phase chromatography on a 240 g C₁₈ column, using 5-35% CH₃CN/H₂O (0.1% formic acid) to afford the title compound (5.12 g, 34%) as a beige solid. ¹H NMR (400 MHz, DMSO-d₆) δ 12.07 (br. s., 1H) 2.55 (br. s., 2H) 2.34-2.41 (m, 2H) 2.28 (s, 3H) 1.66-1.72 (m, 4H). MS-ESI (m/z) calc'd for C₁₁H₁₃N₂₀ [M+H]⁺: 189.1. Found 189.0.

### Step 2: 2-Chloro-4-methyl-5,6,7,8-tetrahydroquinoline-3-carbonitrile

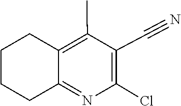

A suspension of 4-methyl-2-oxo-1,2,5,6,7,8-hexahydroquinoline-3-carbonitrile (5.12 g, 27.2 mmol) in phosphorus(V) oxychloride (25.0 mL, 267.4 mmol) was heated at 100° C. for 4 hrs. Excess POCl₃ was evaporated and the brown oily residue was partitioned between H₂O and DCM. The phases were separated and the aqueous layer was extracted with DCM (2×). The combined organic phases were washed with water (1×), dried over anhydrous Na₂SO₄, and evaporated to dryness. The material was purified by silica gel column chromatography using a 0-30% EtOAc/cyclohexane gradient eluent to afford the title compound (1.22 g, 22%), as a white solid. ¹H NMR (400 MHz, DMSO-d₆) δ 2.86 (br. s., 2H) 2.66 (br. s., 2H) 2.43 (s, 3H) 1.74-1.83 (m, 4H). MS-ESI (m/z) calc'd for C₁₁H₁₂ClN₂ [M+H]⁺: 207.1; Found 207.0. 209.0.

### Step 3: 2-Chloro-3-cyano-4-methyl-5,6,7,8-tetrahydroquinoline 1-oxide

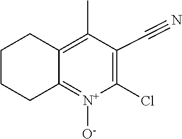

To a solution of 2-chloro-4-methyl-5,6,7,8-tetrahydroquinoline-3-carbonitrile (1.39 g, 6.73 mmol) in trifluoroacetic acid (25 mL) was added a 30 wt. % solution of hydrogen peroxide in water (2.06 mL, 20.18 mmol) and the mixture was stirred at 75° C. for 16 hrs. An additional 1.0 mL of a 30 wt. % solution of hydrogen peroxide in water was added and the mixture was stirred at 75° C. for another 24 hrs. Water was added and the solution was neutralized by addition of solid K₂CO₃ and then extracted with DCM (3×). The combined organic layers were washed with H₂O (1×), passed through a phase separator, and evaporated under reduced pressure to afford the title compound (1.19 g, 79%) as a light yellow solid. ¹H NMR (400 MHz, DMSO-d₆) δ 2.78-2.89 (m, 2H) 2.63-2.74 (m, 2H) 2.41 (s, 3H) 1.67-1.84 (m, 4H). MS-ESI (m/z) calc'd for C₁₁H₁₂ClN[N⁺][O⁻] [M+H]⁺: 223.1. Found 223.0.

### Step 4: 3-Cyano-2-methoxy-4-methyl-5,6,7,8-tetrahydroquinoline 1-oxide

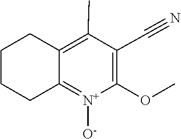 [SMILES: COc1c(C#N)c(C)c2c([n+]1[O-])CCCC2]

To a solution of 2-chloro-3-cyano-4-methyl-5,6,7,8-tetrahydroquinoline 1-oxide (390.0 mg, 1.75 mmol) in MeOH (6 mL) was added sodium methoxide (189.23 mg, 3.5 mmol) and the mixture was stirred at r.t. overnight. The reaction mixture was partitioned between H₂O and EtOAc, the phases were separated, the aqueous layer was extracted with EtOAc (2×), and the combined organic phases were washed with brine (1×), dried over anhydrous Na₂SO₄, and evaporated to dryness. The material was purified by silica gel column chromatography using a 50-100% EtOAc/cyclohexane gradient eluent, and then EtOAc/MeOH 95:5 to afford the title compound (200 mg, 52%) as a light yellow solid. ¹H NMR (400 MHz, DMSO-d₆) δ 4.15 (s, 3H) 2.79 (t, J=6.27 Hz, 2H) 2.63 (t, J=6.05 Hz, 2H) 2.35 (s, 3H) 1.66-1.82 (m, 4H). MS-ESI (m/z) calc'd for C₁₂H₁₅N₂O₂ [M+H]⁺: 219.1. Found 219.1.

### Step 5: 8-Hydroxy-2-methoxy-4-methyl-5,6,7,8-tetrahydroquinoline-3-carbonitrile

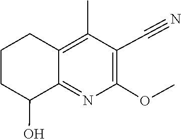 [SMILES: COc1nc2c(c(C)c1C#N)CCCC2O]

To a solution of 3-cyano-2-methoxy-4-methyl-5,6,7,8-tetrahydroquinoline 1-oxide (200.0 mg, 0.92 mmol) in DCM (5 mL) was added dropwise trifluoroacetic anhydride (0.38 mL, 2.75 mmol) and the mixture was stirred at 25° C. for 16 hrs. The solvent was evaporated and the residue was taken up in MeOH. K₂CO₃ was then added till basic pH and the suspension was stirred at 25° C. for 1 hr. The solvent was evaporated and the residue was taken up in H₂O and extracted with DCM (3×). The combined organic layers were passed through a phase separator and evaporated to dryness. The material was purified by silica gel column chromatography using a 0-100% EtOAc/cyclohexane gradient eluent to afford the title compound (45 mg, 22%) as a white solid. ¹H NMR (400 MHz, DMSO-d₆) δ 5.19 (d, J=4.18 Hz, 1H) 4.46-4.52 (m, 1H) 3.98 (s, 3H) 2.57-2.67 (m, 1H) 2.47 (d, J=7.92 Hz, 1H) 2.35-2.40 (m, 3H) 1.67-1.97 (m, 4H). MS-ESI (m/z) calc'd for C₁₂H₁₅N₂O₂ [M+H]⁺: 219.1. Found 219.1.

### Step 6: N-(3-Cyano-2-methoxy-4-methyl-5,6,7,8-tetrahydroquinolin-8-yl)-2-nitro-N-(3-(oxazol-5-yl)-1-((2-(trimethylsilyl)ethoxy)methyl)-1H-indazol-5-yl)benzenesulfonamide

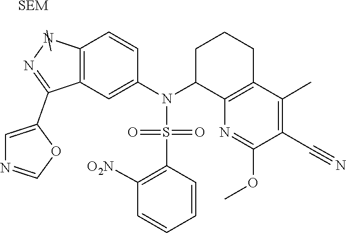

To a solution of 8-hydroxy-2-methoxy-4-methyl-5,6,7,8-tetrahydroquinoline-3-carbonitrile (45.0 mg, 0.21 mmol), 2-nitro-N-[3-(1,3-oxazol-5-yl)-1-(2-trimethylsilylethoxymethyl)indazol-5-yl]benzenesulfonamide (106.31 mg, 0.21 mmol) and triphenylphosphine (108.16 mg, 0.41 mmol) in THF (3 mL) was added diethyl azodicarboxylate (64.93 uL, 0.41 mmol) dropwise and the mixture was stirred at r.t. for 1.5 hrs. An additional 2 eq. of diethyl azodicarboxylate and 2 eq of triphenylphosphine were then added and the mixture was stirred at r.t. overnight. The reaction mixture was partitioned between H₂O and EtOAc and the phases were separated. The aqueous layer was extracted with EtOAc (2×) and the combined organic phases were washed with H₂O (1×), dried over anhydrous Na₂SO₄, and evaporated to dryness. The material was purified by silica gel column chromatography using a 0-50% EtOAc/cyclohexane gradient eluent to afford the title compound (336 mg) as a beige solid. MS-ESI (m/z) calc'd for C₃₄H₃₈N₇O₇SSi [M+H]⁺: 716.2. Found 716.5.

### Step 7: 2-Methoxy-4-methyl-8-((3-(oxazol-5-yl)-1-((2-(trimethylsilyl)ethoxy)methyl)-1H-indazol-5-yl)amino)-5,6,7,8-tetrahydroquinoline-3-carbonitrile

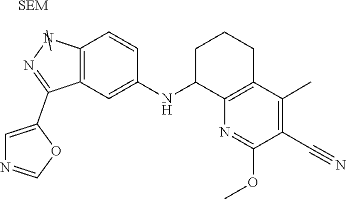

To a solution of N-(3-cyano-2-methoxy-4-methyl-5,6,7,8-tetrahydroquinolin-8-yl)-2-nitro-N-[3-(1,3-oxazol-5-yl)-1-(2-trimethylsilylethoxymethyl)indazol-5-yl]benzenesulfonamide (147.47 mg, 0.21 mmol) in DMF (3 mL) was added K₂CO₃ (0.11 mL, 0.82 mmol) and benzenethiol (68.09 mg, 0.620 mmol) and the mixture was stirred at r.t. for 1 hr. The reaction mixture was partitioned between H₂O and EtOAc and the phases were separated. The aqueous layer was extracted with EtOAc (2×) and the combined organic phases were washed with brine (1×), dried over anhydrous Na₂SO₄, and evaporated to dryness. The residue was purified by SCX using a 2 g cartridge, washing with MeOH and then eluting the compound with a 2 M solution of NH₃ in MeOH to afford the title compound (84 mg, 77%) as a yellow solid. ¹H NMR (400 MHz, DMSO-d₆) δ 8.50 (s, 1H) 7.70 (s, 1H) 7.54 (d, J=9.02 Hz, 1H) 7.24 (d, J=1.76 Hz, 1H) 7.11 (dd, J=9.13, 1.87 Hz, 1H) 5.91 (d, J=7.92 Hz, 1H) 5.70 (s, 2H) 4.67-4.77 (m, 1H) 3.64 (s, 3H) 3.53 (t, J=7.92 Hz, 2H) 2.59-2.74 (m, 2H) 2.39 (s, 3H) 1.78-2.08 (m, 4H) 0.80 (t, J=7.92 Hz, 2H) −0.11 (s, 9H). MS-ESI (m/z) calc'd for C₂₈H₃₅N₆O₃Si [M+H]⁺: 531.3. Found 531.4.

### Step 8: 2-Methoxy-4-methyl-8-[[3-(1,3-oxazol-5-yl)-1H-indazol-5-yl]amino]-5,6,7,8-tetrahydroquinoline-3-carbonitrile, enantiomer 1 and 2

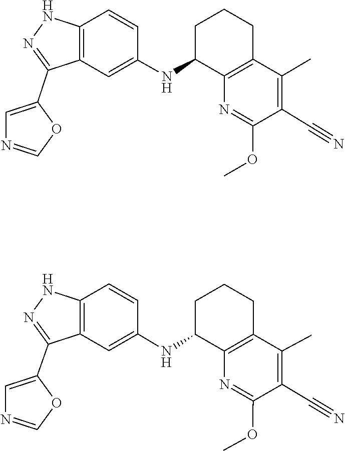 [SMILES: COc1nc2c(c(C)c1C#N)CCC[C@@H]2Nc1ccc2[nH]nc(-c3cnco3)c2c1 ; COc1nc2c(c(C)c1C#N)CCC[C@H]2Nc1ccc2[nH]nc(-c3cnco3)c2c1]

Prepared as described for 1-methoxy-5-((3-(oxazol-5-yl)-1H-indazol-5-yl)amino)-5,6,7,8-tetrahydronaphthalene-2-carbonitrile using 2-methoxy-4-methyl-8-[[3-(1,3-oxazol-5-yl)-1-(2-trimethylsilylethoxymethyl)indazol-5-yl]amino]-5,6,7,8-tetrahydroquinoline-3-carbonitrile in place of 1-methoxy-5-((3-(oxazol-5-yl)-1-((2-(trimethylsilyl)ethoxy)methyl)-1H-indazol-5-yl)amino)-5,6,7,8-tetrahydronaphthalene-2-carbonitrile to afford 2-methoxy-4-methyl-8-[[3-(1,3-oxazol-5-yl)-1H-indazol-5-yl]amino]-5,6,7,8-tetrahydroquinoline-3-carbonitrile (25 mg, 38%), which was subjected to chiral separation using Method GL to afford 2-methoxy-4-methyl-8-[[3-(1,3-oxazol-5-yl)-1H-indazol-5-yl]amino]-5,6,7,8-tetrahydroquinoline-3-carbonitrile, enantiomer 1 (87 mg, 67%) as a yellow solid. ¹H NMR (400 MHz, DMSO-d₆) δ 13.06 (br. s., 1H) 8.46 (s, 1H) 7.62 (s, 1H) 7.35 (d, J=8.80 Hz, 1H) 7.20 (d, J=1.54 Hz, 1H) 7.04 (dd, J=9.02, 1.98 Hz, 1H) 5.80 (d, J=7.48 Hz, 1H) 4.67 (br. s., 1H) 3.68 (s, 3H) 2.61-2.75 (m, 2H) 2.30-2.42 (m, 3H) 1.85-2.08 (m, 4H) MS-ESI (m/z) calc'd for C₂₂H₂₁N₆O₂ [M+H]⁺: 401.2. Found 401.2. A later eluting fraction was also isolated to afford 2-methoxy-4-methyl-8-[[3-(1,3-oxazol-5-yl)-1H-indazol-5-yl]amino]-5,6,7,8-tetrahydroquinoline-3-carbonitrile, enantiomer 2 (8 mg, 12%) as a yellow solid. ¹H NMR (400 MHz, DMSO-d₆) δ 13.05 (br. s., 1H) 8.46 (s, 1H) 7.61-7.65 (m, 1H) 7.35 (d, J=8.80 Hz, 1H) 7.20 (d, J=1.54 Hz, 1H) 7.04 (dd, J=9.02, 2.20 Hz, 1H) 5.80 (d, J=7.70 Hz, 1H) 4.68 (br. s., 1H) 3.68 (s, 3H) 2.58-2.72 (m, 2H) 2.40 (s, 3H) 1.82-2.02 (m, 4H). MS-ESI (m/z) calc'd for C₂₂H₂₁N₆O₂ [M+H]⁺: 401.2. Found 401.2.

### Example 166: 2-Chloro-4-methyl-7-[[3-(1,3-oxazol-5-yl)-1H-indazol-5-yl]oxy]-6,7-dihydro-5H-cyclopenta[b]pyridine-3-carbonitrile, enantiomer 1 and 2

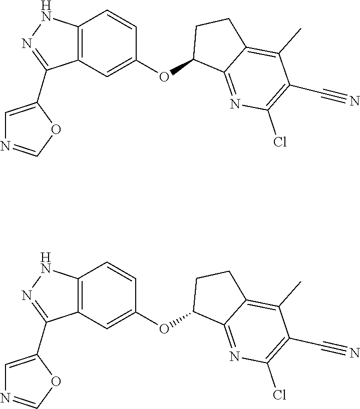 [SMILES: Cc1c(C#N)c(Cl)nc2c1CC[C@@H]2Oc1ccc2[nH]nc(-c3cnco3)c2c1 ; Cc1c(C#N)c(Cl)nc2c1CC[C@H]2Oc1ccc2[nH]nc(-c3cnco3)c2c1]

### Step 1: 2-Chloro-4-methyl-7-((3-(oxazol-5-yl)-1-((2-(trimethylsilyl)ethoxy)methyl)-1H-indazol-5-yl)oxy)-6,7-dihydro-5H-cyclopenta[b]pyridine-3-carbonitrile

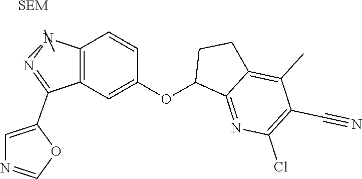

To a cooled (0° C.) solution of 2-chloro-7-hydroxy-4-methyl-6,7-dihydro-5H-cyclopenta[b]pyridine-3-carbonitrile (43.0 mg, 0.21 mmol), 3-(1,3-oxazol-5-yl)-1-(2-trimethylsilylethoxymethyl)indazol-5-ol (68.31 mg, 0.21 mmol) and triphenylphosphine (108.11 mg, 0.41 mmol) in THF (3.515 mL) was added diisopropyl azodicarboxylate (0.05 mL, 0.25 mmol) dropwise and after 10 min the mixture was stirred at r.t. for 18 hrs. The reaction mixture was then diluted with H₂O and EtOAc. The organic phase was separated, dried over Na₂SO₄, filtered, and concentrated under reduced pressure to give a residue. This residue was combined with the residue from an additional synthesis performed at 0.1 mmol scale. The combined residues were purified by silica gel column chromatography using a 0-100% EtOAc/cyclohexane gradient eluent to afford the title compound (155 mg), as a colorless oil. ¹H NMR (400 MHz, DMSO-d₆) δ 8.56 (s, 1H), 7.92 (s, 1H), 7.81-7.76 (m, 1H), 7.72-7.70 (m, 1H), 7.28 (dd, J=9.1, 2.3 Hz, 1H), 6.02 (dd, J=7.2, 4.4 Hz, 1H), 5.81 (s, 2H), 3.60-3.54 (m, 2H), 3.16-3.03 (m, 1H), 3.01-2.89 (m, 1H), 2.83-2.70 (m, 1H), 2.54 (s, 3H), 2.28-2.12 (m, 1H), 0.90-0.74 (m, 2H), −0.10 (s, 9H). MS-ESI (m/z) calc'd for C₂₆H₂₉ClN₅O₃Si [M+H]⁺: 522.2. Found 522.2.

### Step 2: 2-Chloro-4-methyl-7-[[3-(1,3-oxazol-5-yl)-1H-indazol-5-yl]oxy]-6,7-dihydro-5H-cyclopenta[b]pyridine-3-carbonitrile, enantiomer 1 and 2

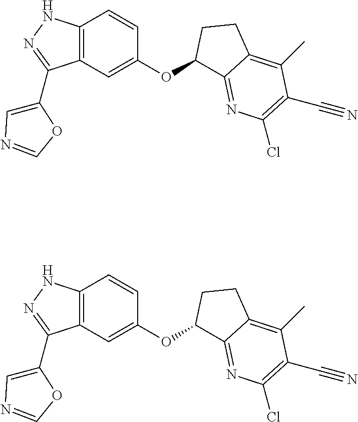 [SMILES: Cc1c(C#N)c(Cl)nc2c1CC[C@@H]2Oc1ccc2[nH]nc(-c3cnco3)c2c1 ; Cc1c(C#N)c(Cl)nc2c1CC[C@H]2Oc1ccc2[nH]nc(-c3cnco3)c2c1]

Prepared as described for 1-methoxy-5-((3-(oxazol-5-yl)-1H-indazol-5-yl)amino)-5,6,7,8-tetrahydronaphthalene-2-carbonitrile using 2-chloro-4-methyl-7-[3-(1,3-oxazol-5-yl)-1-(2-trimethylsilylethoxymethyl)indazol-5-yl]oxy-6,7-dihydro-5H-cyclopenta[b]pyridine-3-carbonitrile in place of 1-methoxy-5-((3-(oxazol-5-yl)-1-((2-(trimethylsilyl)ethoxy)methyl)-1H-indazol-5-yl)amino)-5,6,7,8-tetrahydronaphthalene-2-carbonitrile to afford 2-chloro-4-methyl-7-[[3-(1,3-oxazol-5-yl)-1H-indazol-5-yl]oxy]-6,7-dihydro-5H-cyclopenta[b]pyridine-3-carbonitrile (29 mg, 26%), which was subjected to chiral separation using Method GM to afford 2-chloro-4-methyl-7-[[3-(1,3-oxazol-5-yl)-1H-indazol-5-yl]oxy]-6,7-dihydro-5H-cyclopenta[b]pyridine-3-carbonitrile, enantiomer 1 (9 mg, 8%) as a yellow solid. ¹H NMR (400 MHz, DMSO-d₆) δ 13.41 (s, 1H), 8.51 (s, 1H), 7.82 (s, 1H), 7.66 (d, J=2.3 Hz, 1H), 7.56 (d, J=9.1 Hz, 1H), 7.17 (dd, J=9.0, 2.3 Hz, 1H), 5.97 (dd, J=7.3, 4.4 Hz, 1H), 3.15-3.02 (m, 1H), 2.99-2.88 (m, 1H), 2.80-2.68 (m, 1H), 2.53 (s, 3H), 2.24-2.12 (m, 1H). MS-ESI (m/z) calc'd for C₂₀H₁₅ClNO₃ [M+H]⁺: 392.1 Found 392.1. A later eluting fraction was also isolated to afford 2-chloro-4-methyl-7-[[3-(1,3-oxazol-5-yl)-1H-indazol-5-yl]oxy]-6,7-dihydro-5H-cyclopenta[b]pyridine-3-carbonitrile, enantiomer 2 (8 mg, 7%) as a yellow solid. ¹H NMR (400 MHz, DMSO-d₆) δ 13.41 (s, 1H), 8.51 (s, 1H), 7.82 (s, 1H), 7.66 (d, J=2.3 Hz, 1H), 7.56 (d, J=9.1 Hz, 1H), 7.17 (dd, J=9.0, 2.3 Hz, 1H), 5.97 (dd, J=7.2, 4.4 Hz, 1H), 3.15-3.02 (m, 1H), 3.00-2.87 (m, 1H), 2.79-2.68 (m, 1H), 2.53 (s, 3H), 2.24-2.12 (m, 1H). MS-ESI (m/z) calc'd for C₂₀H₁₅ClNO₃ [M+H]⁺: 392.1. Found 392.1.

### Example 167: 4,6-Difluoro-1-[[3-(1,3-oxazol-5-yl)-1H-indazol-5-yl]oxy]-2,3-dihydro-1H-indene-5-carbonitrile, enantiomer 1 and 2

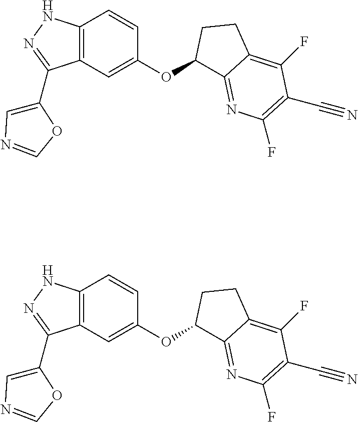 [SMILES: N#Cc1c(F)nc2c(c1F)CC[C@@H]2Oc1ccc2[nH]nc(-c3cnco3)c2c1 ; N#Cc1c(F)nc2c(c1F)CC[C@H]2Oc1ccc2[nH]nc(-c3cnco3)c2c1]

### Step 1: 3-(3-Bromo-2,4-difluorophenyl)propanoic acid

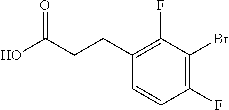 [SMILES: O=C(O)CCc1ccc(F)c(Br)c1F]

Triethylamine (0.15 mL, 1.09 mmol) was added to a solution of 3-bromo-2,4-difluorobenzaldehyde (200.0 mg, 0.90 mmol) and 2,2-dimethyl-1,3-dioxane-4,6-dione (0.13 g, 0.90 mmol) in DMF (0.700 mL). The solution was cooled to 0° C. and formic acid (0.1 mL, 2.71 mmol) was added. The flask was heated with stirring at 100° C. for 18 hrs. The reaction mixture was then cooled to r.t. and an aqueous solution of NaHCO₃ (250 mL) was added and the mixture was washed with EtOAc (250 mL). The aqueous phase was acidified with 2 M HCl until pH 3, and then extracted with EtOAc (250 mL). The organic phase was concentrated under reduced pressure to afford the title compound (2.367 g, 98%) as a colorless oil. ¹H NMR (400 MHz, DMSO-d₆) δ 12.19 (s, 1H), 7.40 (td, J=8.6, 6.3 Hz, 1H), 7.20 (td, J=8.5, 1.7 Hz, 1H), 2.86 (t, J=7.7 Hz, 2H), 2.54 (t, J=7.6 Hz, 2H). MS-ESI (m/z) calc'd for C₉H₈BrF₂O₂ [M+H]⁺: 265.0, 267.0. Found [M+H]⁻: 263.0, 265.1.

### Step 2: 3-(3-Bromo-2,4-difluorophenyl)propanoyl chloride

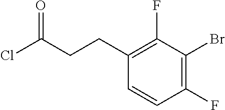 [SMILES: O=C(Cl)CCc1ccc(F)c(Br)c1F]

3-(3-Bromo-2,4-difluorophenyl)propanoic acid (205.0 mg, 0.77 mmol) was dissolved in DCM (1 mL) and 2 drops of DMF were added. Oxalyl chloride (0.09 mL, 1.08 mmol) was then added dropwise to the mixture. After stirring at r.t. for 2 hrs, the solvent was removed to afford the title compound (219 mg, 99%), as a dark yellow solid which was used without further purification. MS-ESI (m/z) calc'd for C₉H₇BrClF₂O [M+H]⁺: 282.9. Found 282.9.

### Step 3: 5-Bromo-4,6-difluoro-2,3-dihydro-1H-inden-1-one

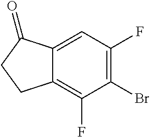 [SMILES: O=C1CCc2c1cc(F)c(Br)c2F]

3-(3-Bromo-2,4-difluorophenyl)propanoyl chloride (2.54 g, 8.94 mmol) was dissolved in DCM (28.6 mL). AlCl₃ (1.19 g, 8.94 mmol) was then added portionwise and the reaction mixture was refluxed with stirring under an N₂ atmosphere for 18 hrs. After cooling to r.t., the reaction mixture was carefully poured into a 1:1 solution of 2 M HCl and ice water (50 mL+50 mL) with agitation. The resulting solution was then extracted with DCM (3×50 mL). The combined organic phases were dried over Na₂SO₄, filtered, and then concentrated under reduced pressure. The residue was purified by silica gel column chromatography using a 0-50% EtOAc/cyclohexane gradient eluent to afford the title compound (833 mg, 38%), as a white solid. ¹H NMR (400 MHz, DMSO-d₆) δ 7.50 (dd, J=6.8, 1.2 Hz, 1H), 3.17-3.07 (m, 2H), 2.77-2.69 (m, 2H). MS-ESI (m/z) calc'd for C₉H₆BrF₂₀ [M+H]⁺: 247.0, 249.0. Found 247.0; 249.0.

### Step 4: 5-Bromo-4,6-difluoro-2,3-dihydro-1H-inden-1-ol

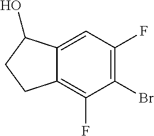 [SMILES: OC1CCc2c1cc(F)c(Br)c2F]

To a solution of 5-bromo-4,6-difluoro-2,3-dihydro-1H-inden-1-one (200.0 mg, 0.79 mmol) in MeOH (4 mL) was added sodium borohydride (60.03 mg, 1.59 mmol) and the mixture was stirred at room temperature for 1 hr. Then the mixture was concentrated under reduced pressure to give a residue that was taken up in EtOAc and H₂O. The organic phase was separated, dried over Na₂SO₄, filtered, and concentrated under reduced pressure. The material was purified by silica gel column chromatography using a 0-50% EtOAc/cyclohexane gradient eluent to afford the title compound (172 mg, 87%), as a colorless oil. ¹H NMR (400 MHz, DMSO-d₆) δ 7.14 (d, J=7.8 Hz, 1H), 5.73 (d, J=5.9 Hz, 1H), 5.05 (q, J=6.4 Hz, 1H), 2.95 (ddd, J=16.3, 8.8, 3.9 Hz, 1H), 2.81-2.69 (m, 1H), 2.46-2.35 (m, 1H), 1.90-1.76 (m, 1H). MS-ESI (m/z) calc'd for C₉H₈BrF₂O [M+H−H₂O]⁺: 231.0, 233.0. Found 230.9, 233.0.

### Step 5: 5-(5-((5-Bromo-4,6-difluoro-2,3-dihydro-1H-inden-1-yl)oxy)-1-((2-(trimethylsilyl)ethoxy)methyl)-1H-indazol-3-yl)oxazole

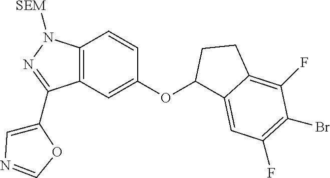 [SMILES: C[Si](C)(C)CCOCn1nc(-c2cnco2)c2cc(OC3CCc4c3cc(F)c(Br)c4F)ccc21]

Diisopropyl azodicarboxylate (0.06 mL, 0.280 mmol) was added dropwise to a cooled (0° C.) solution of 5-bromo-4,6-difluoro-2,3-dihydro-1H-inden-1-ol (70.0 mg, 0.280 mmol), 3-(1,3-oxazol-5-yl)-1-(2-trimethylsilylethoxymethyl)indazol-5-ol (93.16 mg, 0.280 mmol) and triphenylphosphine (147.44 mg, 0.560 mmol) in THF (5.723 mL). After 10 min the reaction mixture was brought to r.t. and stirred for 3 hrs. Water was added and the mixture was extracted with EtOAc (3×). The organic layers were collected, dried over Na₂SO₄, filtered, and concentrated under reduced pressure. The residue was purified by silica gel column chromatography using a 0-100% EtOAc/cyclohexane gradient eluent to afford the title compound (99 mg, 62%), as a pale yellow oil. MS-ESI (m/z) calc'd for C₂₅H₂₇BrF₂N₃O₃Si [M+H]⁺: 562.1, 564.1. Found 562.1, 564.2.

### Step 6: 4,6-Difluoro-1-((3-(oxazol-5-yl)-1-((2-(trimethylsilyl)ethoxy)methyl)-1H-indazol-5-yl)oxy)-2,3-dihydro-1H-indene-5-carbonitrile

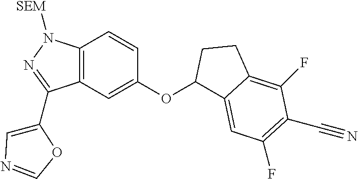 [SMILES: C[Si](C)(C)CCOCn1nc(-c2cnco2)c2cc(OC3CCc4c3cc(F)c(C#N)c4F)ccc21]

In a sealed microwave vial, potassium hexacyanoferrate (II), 0.1 N standardized solution (1.76 mL, 0.180 mmol), 5-(5-((5-bromo-4,6-difluoro-2,3-dihydro-1H-inden-1-yl)oxy)-1-((2-(trimethylsilyl)ethoxy)methyl)-1H-indazol-3-yl)oxazole (99.0 mg, 0.18 mmol) and KOAc (17.27 mg, 0.18 mmol) were dissolved in a mixture of 1,4-dioxane (1.4 mL) and H₂O (0.5 mL). The mixture was degassed with N₂ for 15 minutes. Then XPhos (18.49 mg, 0.040 mmol) and XPhos Pd G3 (14.9 mg, 0.020 mmol) were added and the mixture was stirred at 100° C. for 2 hrs. The reaction mixture was cooled to r.t. and then partitioned between H₂O and EtOAc. The phases were separated and the aqueous layer was extracted with EtOAc (2×). The combined organic phases were washed with H₂O (1×), dried over anhydrous Na₂SO₄, filtered, and evaporated to dryness. The material was purified by silica gel column chromatography using a 0-100% EtOAc/cyclohexane gradient eluent to afford the title compound (39 mg, 39%) as a yellow solid. ¹H NMR (400 MHz, DMSO-d₆) δ 8.55 (s, 1H), 7.94 (s, 1H), 7.79 (d, J=9.1 Hz, 1H), 7.62 (d, J=2.2 Hz, 1H), 7.50 (d, J=8.4 Hz, 1H), 7.28 (dd, J=9.0, 2.2 Hz, 1H), 6.13 (t, J=5.9 Hz, 1H), 5.80 (s, 2H), 3.56 (t, J=7.9 Hz, 2H), 3.16-3.08 (m, 1H), 3.04-2.91 (m, 1H), 2.83-2.72 (m, 2H), 0.81 (t, J=8.0 Hz, 2H), −0.11 (s, 9H). MS-ESI (m/z) calc'd for C₂₆H₂₇F₂N₄O₃Si [M+H]⁺: 509.2. Found 509.3.

### Step 7: 4,6-Difluoro-1-[[3-(1,3-oxazol-5-yl)-1H-indazol-5-yl]oxy]-2,3-dihydro-1H-indene-5-carbonitrile, enantiomer 1 and 2

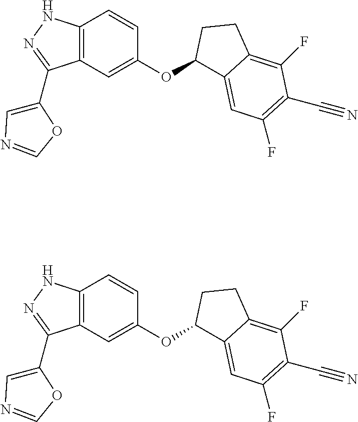 [SMILES: N#Cc1c(F)cc2c(c1F)CC[C@@H]2Oc1ccc2[nH]nc(-c3cnco3)c2c1 ; N#Cc1c(F)cc2c(c1F)CC[C@H]2Oc1ccc2[nH]nc(-c3cnco3)c2c1]

Prepared as described for 1-methoxy-5-((3-(oxazol-5-yl)-1H-indazol-5-yl)amino)-5,6,7,8-tetrahydronaphthalene-2-carbonitrile using 4,6-difluoro-1-[3-(1,3-oxazol-5-yl)-1-(2-trimethylsilylethoxymethyl)indazol-5-yl]oxy-2,3-dihydro-1H-indene-5-carbonitrile in place of 1-methoxy-5-((3-(oxazol-5-yl)-1-((2-(trimethylsilyl)ethoxy)methyl)-1H-indazol-5-yl)amino)-5,6,7,8-tetrahydronaphthalene-2-carbonitrile to afford 4,6-difluoro-1-[[3-(1,3-oxazol-5-yl)-1H-indazol-5-yl]oxy]-2,3-dihydro-1H-indene-5-carbonitrile (29 mg, 26%), which was subjected to chiral separation using Method GN to afford 4,6-difluoro-1-[[3-(1,3-oxazol-5-yl)-1H-indazol-5-yl]oxy]-2,3-dihydro-1H-indene-5-carbonitrile, enantiomer 1 (2.2 mg, 8%) as a white solid. ¹H NMR (400 MHz, MeOD) δ 8.36 (s, 1H), 7.68 (s, 1H), 7.60 (d, J=2.3 Hz, 1H), 7.55 (dd, J=9.1, 0.7 Hz, 1H), 7.27 (d, J=8.2 Hz, 1H), 7.21 (dd, J=9.1, 2.3 Hz, 1H), 6.01 (t, J=6.1 Hz, 1H), 3.24-3.14 (m, 1H), 3.02 (dt, J=16.0, 7.4 Hz, 1H), 2.88-2.74 (m, 1H), 2.37-2.22 (m, 1H). MS-ESI (m/z) calc'd for C₂₀H₁₃F₂N₄O₂ [M+H]⁺: 379.1. Found 379.1. A later eluting fraction was also isolated to afford 4,6-difluoro-1-[[3-(1,3-oxazol-5-yl)-1H-indazol-5-yl]oxy]-2,3-dihydro-1H-indene-5-carbonitrile, enantiomer 2 (2.2 mg, 8%) as a white solid. ¹H NMR (400 MHz, MeOD) δ 8.36 (s, 1H), 7.68 (s, 1H), 7.60 (d, J=2.3 Hz, 1H), 7.55 (dd, J=9.1, 0.7 Hz, 1H), 7.27 (d, J=8.2 Hz, 1H), 7.21 (dd, J=9.1, 2.3 Hz, 1H), 6.01 (t, J=6.1 Hz, 1H), 3.24-3.14 (m, 1H), 3.02 (dt, J=16.1, 7.4 Hz, 1H), 2.89-2.75 (m, 1H), 2.36-2.22 (m, 1H). MS-ESI (m/z) calc'd for C₂₀H₁₃F₂N₄O₂ [M+H]⁺: 379.1. Found 379.1.

### Example 168: trans-3-Methyl-1-[[3-(1,3-oxazol-5-yl)-1H-indazol-5-yl]amino]-2,3-dihydro-1H-indene-5-carbonitrile, enantiomer 1 and 2

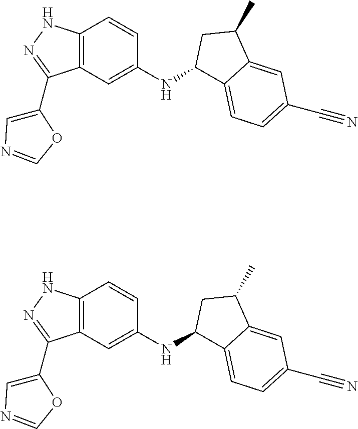 [SMILES: C[C@@H]1C[C@@H](Nc2ccc3[nH]nc(-c4cnco4)c3c2)c2ccc(C#N)cc21 ; C[C@H]1C[C@H](Nc2ccc3[nH]nc(-c4cnco4)c3c2)c2ccc(C#N)cc21]

### Step 1: 5-Bromo-3-methyl-2,3-dihydro-1H-inden-1-one

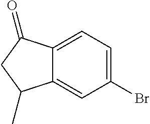

A mixture of NaCl (615.0 mg, 10.52 mmol) and AlCl₃ (2.5 g, 18.75 mmol) was stirred at 130° C. for 15 min; then 1-(4-bromophenyl)-4-chlorobutan-1-one (500.0 mg, 1.91 mmol) was added and the resulting mixture was heated to 180° C. and stirred for 30 minutes. The mixture was allowed to cool to r.t. and quenched by portionwise addition to a cold 1 N HCl solution (100 mL). The mixture was extracted with DCM. The combined organic layers were separated, dried over Na₂SO₄, filtered, and concentrated. The material was purified by silica gel column chromatography using a 0-50% EtOAc/cyclohexane gradient eluent to afford the title compound (430 mg, 99%), as a yellow oil. ¹H NMR (400 MHz, DMSO-d₆) δ 7.99-7.91 (m, 1H), 7.62 (ddd, J=8.1, 1.7, 0.7 Hz, 1H), 7.54 (d, J=8.1 Hz, 1H), 3.49-3.38 (m, 1H), 2.89 (dd, J=19.0, 7.6 Hz, 1H), 2.24 (dd, J=19.0, 3.6 Hz, 1H), 1.34 (d, J=7.1 Hz, 3H); MS-ESI (m/z) calc'd for C₁₀H₁₀BrO [M+H]⁺: 225.0, 227.0. Found 225.0, 227.0.

### Step 2: 3-Methyl-1-oxo-2,3-dihydro-1H-indene-5-carbonitrile

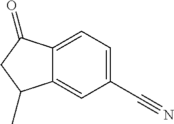

In a sealed microwave vial, potassium hexacyanoferrate (II), 0.1 N standardized solution (19.1 mL, 1.91 mmol), 5-bromo-3-methyl-2,3-dihydro-1H-inden-1-one (430.0 mg, 1.91 mmol) and KOAc (187.49 mg, 1.91 mmol) were dissolved in a mixture of H₂O (1.9 mL) and 1,4-dioxane (19.05 mL). The mixture was degassed with N₂ for 15 minutes. Then XPhos (18.49 mg, 0.04 mmol) and XPhos Pd G3 (32.84 mg, 0.04 mmol) were added and the mixture was left stirring at 100° C. for 3 hrs. The reaction mixture was partitioned between H₂O and EtOAc. The phases were separated and the aqueous layer was extracted with EtOAc (2×). The combined organic phases were washed with H₂O (1×), dried over anhydrous Na₂SO₄, and evaporated to dryness. The residue was purified by silica gel column chromatography using a 0-40% EtOAc/cyclohexane gradient eluent to afford the title compound the title (128 mg, 39%) as a white solid. ¹H NMR (400 MHz, DMSO-d₆) δ 8.24 (q, J=1.0 Hz, 1H), 7.87 (dt, J=7.7, 1.0 Hz, 1H), 7.76 (d, J=7.9 Hz, 1H), 3.49 (td, J=7.2, 3.7 Hz, 1H), 2.97 (dd, J=19.2, 7.6 Hz, 1H), 2.32 (dd, J=19.2, 3.7 Hz, 1H), 1.37 (d, J=7.1 Hz, 3H). MS-ESI (m/z) calc'd for C₁₁H₁₀NO [M+H]⁺: 172.1. Found 171.9.

### Step 3: 1-Hydroxy-3-methyl-2,3-dihydro-1H-indene-5-carbonitrile

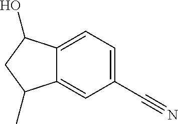

To a solution of 3-methyl-1-oxo-2,3-dihydro-1H-indene-5-carbonitrile (127.0 mg, 0.74 mmol) in MeOH (7 mL) was added sodium borohydride (56.13 mg, 1.48 mmol) and the mixture was stirred at r.t. for 1 hr. After evaporation of the solvent, the residue was taken up in EtOAc and H₂O. The organic phase was dried over Na₂SO₄, filtered, and concentrated to dryness. The residue was purified by silica gel column chromatography using a 0-50% EtOAc/cyclohexane gradient eluent to afford the title compound (124 mg, 96%), as a white solid. ¹H NMR (400 MHz, DMSO-d₆) δ 7.70 (s, 1H), 7.67 (dt, J=7.8, 1.2 Hz, 1H), 7.47 (d, J=7.8 Hz, 1H), 5.56 (d, J=6.1 Hz, 1H), 5.02 (q, J=7.1 Hz, 1H), 3.07-2.94 (m, 1H), 2.62 (dt, J=12.2, 7.1 Hz, 1H), 1.44-1.34 (m, 1H), 1.30 (d, J=6.8 Hz, 3H). MS-ESI (m/z) calc'd for C₁₁H₁₂NO [M+H]⁺: 174.1. Found 174.0.

### Step 4: N-(5-Cyano-3-methyl-2,3-dihydro-1H-inden-1-yl)-2-nitro-N-(3-(oxazol-5-yl)-1-((2-(trimethylsilyl)ethoxy)methyl)-1H-indazol-5-yl)benzenesulfonamide

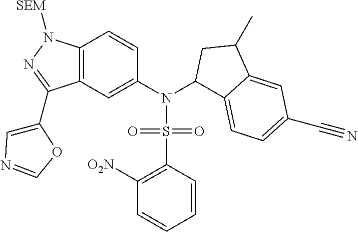 [SMILES: CC1CC(N(c2ccc3c(c2)c(-c2cnco2)nn3COCC[Si](C)(C)C)S(=O)(=O)c2ccccc2[N+](=O)[O-])c2ccc(C#N)cc21]

To a solution of 1-hydroxy-3-methyl-2,3-dihydro-1H-indene-5-carbonitrile (123.0 mg, 0.71 mmol), 2-nitro-N-[3-(1,3-oxazol-5-yl)-1-(2-trimethylsilylethoxymethyl)indazol-5-yl]benzenesulfonamide (366.15 mg, 0.71 mmol) and triphenylphosphine (372.52 mg, 1.42 mmol) in THF (8.07 mL) was added diethyl azodicarboxylate (0.22 mL, 1.42 mmol) dropwise and the mixture was stirred at 25° C. for 5 hrs. The solvent was evaporated, the residue was purified by silica gel column chromatography using a 0-100% EtOAc/cyclohexane gradient eluent to afford the title compound (480 mg, 100%) as an orange solid. MS-ESI (m/z) calc'd for C₃₃H₃₅N₆O₆SSi [M+H]⁺: 671.2. Found: 671.4.

### Step 5: 3-Methyl-1-((3-(oxazol-5-yl)-1-((2-(trimethylsilyl)ethoxy)methyl)-1H-indazol-5-yl)amino)-2,3-dihydro-1H-indene-5-carbonitrile

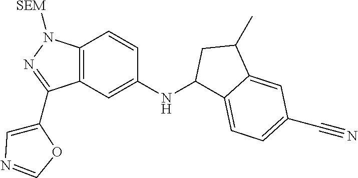 [SMILES: CC1CC(Nc2ccc3c(c2)c(-c2cnco2)nn3COCC[Si](C)(C)C)c2ccc(C#N)cc21]

To a solution of N-(5-Cyano-3-methyl-2,3-dihydro-1H-inden-1-yl)-2-nitro-N-(3-(oxazol-5-yl)-1-((2-(trimethylsilyl)ethoxy)methyl)-1H-indazol-5-yl)benzenesulfonamide (480.0 mg, 0.72 mmol) in DMF (12.26 mL) was added K₂CO₃ (395.59 mg, 2.86 mmol) and benzenethiol (0.22 mL, 2.15 mmol) and the mixture was stirred at 25° C. for 2 hrs. Water was added and the mixture was extracted with EtOAc (3×). The combined organic layers were evaporated to give a yellow oil which was passed through an SCX cartridge to afford the title compound (280 mg, 81%) as a yellow oil. MS-ESI (m/z) calc'd for C₂₇H₃₂N₅O₂Si [M+H]⁺: 486.2. Found 486.3.

### Step 6: trans-3-Methyl-1-[[3-(1,3-oxazol-5-yl)-1H-indazol-5-yl]amino]-2,3-dihydro-1H-indene-5-carbonitrile, enantiomer 1 and 2

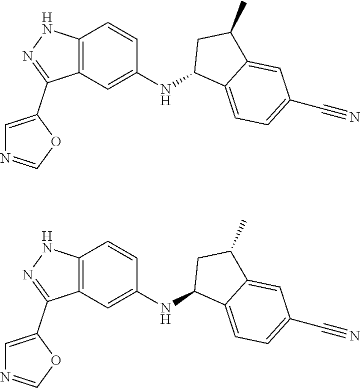 [SMILES: C[C@@H]1C[C@@H](Nc2ccc3[nH]nc(-c4cnco4)c3c2)c2ccc(C#N)cc21 ; C[C@H]1C[C@H](Nc2ccc3[nH]nc(-c4cnco4)c3c2)c2ccc(C#N)cc21]

Prepared as described for 1-methoxy-5-((3-(oxazol-5-yl)-1H-indazol-5-yl)amino)-5,6,7,8-tetrahydronaphthalene-2-carbonitrile using 3-methyl-1-[[3-(1,3-oxazol-5-yl)-1-(2-trimethylsilylethoxymethyl)indazol-5-yl]amino]-2,3-dihydro-1H-indene-5-carbonitrile in place of 1-methoxy-5-((3-(oxazol-5-yl)-1-((2-(trimethylsilyl)ethoxy)methyl)-1H-indazol-5-yl)amino)-5,6,7,8-tetrahydronaphthalene-2-carbonitrile to afford a mixture of four isomers. The material was purified using Method GO to separate the diastereomers. The minor pair was the cis pair of enantiomers (6 mg, 3%), the major pair was the trans pair of enantiomers (101 mg, 49%). The major pair was subjected to chiral separation using Method GP to afford trans-3-methyl-1-[[3-(1,3-oxazol-5-yl)-1H-indazol-5-yl]amino]-2,3-dihydro-1H-indene-5-carbonitrile, enantiomer 1 (24.7 mg, 12%) as a white solid. ¹H NMR (400 MHz, DMSO-d₆) δ 13.10 (s, 1H), 8.46 (s, 1H), 7.79 (s, 1H), 7.67 (s, 1H), 7.63 (dd, J=7.7, 1.5 Hz, 1H), 7.49 (d, J=7.8 Hz, 1H), 7.38 (d, J=8.9 Hz, 1H), 7.10 (d, J=2.0 Hz, 1H), 6.99 (dd, J=9.0, 2.1 Hz, 1H), 5.91 (d, J=8.7 Hz, 1H), 5.33-5.20 (m, 1H), 3.50-3.37 (m, 1H), 2.21 (ddd, J=13.0, 7.8, 5.2 Hz, 1H), 2.10 (ddd, J=12.8, 7.2, 5.5 Hz, 1H), 1.31 (d, J=7.0 Hz, 3H). MS-ESI (m/z) calc'd for C₂₁H₁₈N₅O [M+H]⁺: 356.1 Found 356.2. A later eluting fraction was also isolated to afford trans-3-methyl-1-[[3-(1,3-oxazol-5-yl)-1H-indazol-5-yl]amino]-2,3-dihydro-1H-indene-5-carbonitrile, enantiomer 2 (25.6 mg, 12%) as a white solid. ¹H NMR (400 MHz, DMSO-d₆) δ 13.10 (s, 1H), 8.46 (s, 1H), 7.78 (s, 1H), 7.67 (s, 1H), 7.63 (dd, J=7.8, 1.5 Hz, 1H), 7.48 (d, J=7.8 Hz, 1H), 7.37 (d, J=8.9 Hz, 1H), 7.10 (d, J=2.0 Hz, 1H), 6.98 (dd, J=9.0, 2.1 Hz, 1H), 5.90 (d, J=8.7 Hz, 1H), 5.27 (q, J=7.3 Hz, 1H), 3.47-3.37 (m, 1H), 2.21 (ddd, J=12.9, 7.7, 5.2 Hz, 1H), 2.10 (ddd, J=12.7, 7.2, 5.6 Hz, 1H), 1.31 (d, J=7.0 Hz, 3H). MS-ESI (m/z) calc'd for C₂₁H₁₈N₅O [M+H]⁺: 356.1 Found 356.2.

### Example 169: cis-3-Methyl-1-[[3-(1,3-oxazol-5-yl)-1H-indazol-5-yl]amino]-2,3-dihydro-1H-indene-5-carbonitrile, enantiomer 1 and 2

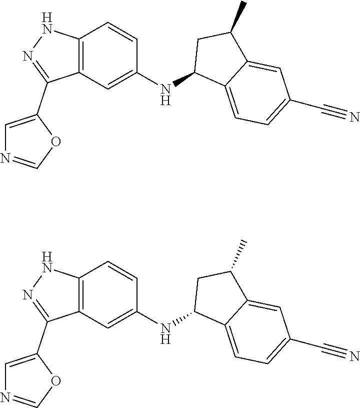 [SMILES: C[C@@H]1C[C@H](Nc2ccc3[nH]nc(-c4cnco4)c3c2)c2ccc(C#N)cc21 ; C[C@H]1C[C@@H](Nc2ccc3[nH]nc(-c4cnco4)c3c2)c2ccc(C#N)cc21]

The cis-3-methyl-1-[[3-(1,3-oxazol-5-yl)-1H-indazol-5-yl]amino]-2,3-dihydro-1H-indene-5-carbonitrile minor pair of diastereomers (6 mg, 3) was subjected to chiral separation using Method GQ to afford cis-3-methyl-1-[[3-(1,3-oxazol-5-yl)-1H-indazol-5-yl]amino]-2,3-dihydro-1H-indene-5-carbonitrile, enantiomer 1 (2.2 mg, 37%) as a white solid. ¹H NMR (400 MHz, MeOD) δ 8.30 (s, 1H), 7.63 (s, 1H), 7.55 (s, 1H), 7.53 (s, OH), 7.49 (d, J=7.8 Hz, 1H), 7.41 (d, J=9.0 Hz, 1H), 7.20 (d, J=2.1 Hz, 1H), 7.09 (dd, J=9.0, 2.1 Hz, 1H), 5.16 (dd, J=9.6, 7.1 Hz, 1H), 3.28-3.17 (m, 1H), 2.94 (dt, J=12.2, 7.1 Hz, 1H), 1.55 (dt, J=12.3, 9.9 Hz, 1H), 1.41 (d, J=6.7 Hz, 3H). MS-ESI (m/z) calc'd for C₂₁H₁₈N₅O [M+H]⁺: 356.1 Found 356.2. A later eluting fraction was also isolated to afford cis-3-methyl-1-[[3-(1,3-oxazol-5-yl)-1H-indazol-5-yl]amino]-2,3-dihydro-1H-indene-5-carbonitrile, enantiomer 2 (1.7 mg, 28%) as a white solid. ¹H NMR (400 MHz, MeOD) δ 8.31 (s, 1H), 7.65 (s, 1H), 7.57 (s, 1H), 7.55 (s, 1H), 7.51 (d, J=7.8 Hz, 1H), 7.43 (d, J=9.0 Hz, 1H), 7.22 (d, J=2.1 Hz, 1H), 7.11 (dd, J=9.0, 2.1 Hz, 1H), 5.27-5.08 (m, 1H), 3.30-3.21 (m, OH), 2.95 (dt, J=12.3, 7.1 Hz, 1H), 1.57 (dt, J=12.3, 9.9 Hz, 1H), 1.43 (d, J=6.8 Hz, 3H). MS-ESI (m/z) calc'd for C₂₁H₁₈N₅O [M+H]⁺: 356.1. Found 356.2.

### Example 170: 2,4-Dimethyl-7-[[3-(1,3-oxazol-5-yl)-1H-indazol-5-yl]amino]-6,7-dihydro-5H-cyclopenta[b]pyridine-3-carbonitrile

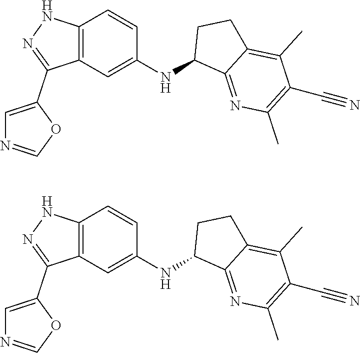 [SMILES: Cc1nc2c(c(C)c1C#N)CC[C@@H]2Nc1ccc2[nH]nc(-c3cnco3)c2c1 ; Cc1nc2c(c(C)c1C#N)CC[C@H]2Nc1ccc2[nH]nc(-c3cnco3)c2c1]

### Step 1: 2-Methyl-4-oxo-4a,5,6,7-tetrahydro-4H-cyclopenta[b]pyridine-3-carbonitrile

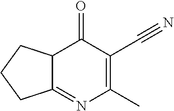

To a solution of 2-acetyl-1-cyclopentanone (5.05 g, 40 mmol) and 2-cyanoacetamide (3.36 g, 40 mmol) in EtOH (100 mL) was added piperidine (3.95 mL, 40 mmol) and the mixture was stirred at 75° C. for 22 hrs. After cooling the solid was filtered to afford the title compound (3.35 g) that was used without further purification. MS-ESI (m/z) calc'd for C₁₀H₁₁N₂O [M+H]⁺: 175.1. Found 175.0.

### Step 2: 4-Chloro-2-methyl-4a,5,6,7-tetrahydro-4H-cyclopenta[b]pyridine-3-carbonitrile

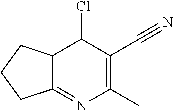

A suspension of 2-methyl-4-oxo-4a,5,6,7-tetrahydro-4H-cyclopenta[b]pyridine-3-carbonitrile (3.35 g, 19.23 mmol) in phosphorus(V) oxychloride (20.0 mL, 213.92 mmol) was heated at 100° C. for 17 hrs. The excess POCl₃ was evaporated and the oil was taken up H₂O and stirred for 30 minutes. The solid that formed was filtered and dried under vacuum to afford the title compound (3.70 g) as an off-white solid that was used without further purification. MS-ESI (m/z) calc'd for C₁₀H₁₀ClN₂ [M+H]⁺: 193.1. Found 193.0.

### Step 3: 4-Chloro-3-cyano-2-methyl-6,7-dihydro-5H-cyclopenta[b]pyridine 1-oxide

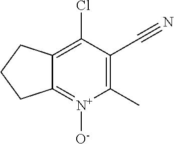

To a solution of 4-chloro-2-methyl-4a,5,6,7-tetrahydro-4H-cyclopenta[b]pyridine-3-carbonitrile (2.0 g, 10.38 mmol) in trifluoroacetic acid (42 mL) was added a 30 wt. % solution of hydrogen peroxide in water (1.29 mL, 12.64 mmol) and the mixture was stirred at 75° C. for 72 hrs. The reaction mixture was brought to r.t. and then concentrated under reduced pressure. Water was added and the solution was neutralized by addition of solid NaHCO₃ and then extracted with DCM (3×). The combined organic layers were passed through a phase separator and evaporated to afford the title compound (1.84 g) as a dark grey solid that was used without further purification. MS-ESI (m/z) calc'd for C₁₀H₁₀ClN₂₀ [M+H]⁺: 209.0. Found 209.0.

### Step 4: 4-Chloro-7-hydroxy-2-methyl-6,7-dihydro-5H-cyclopenta[b]pyridine-3-carbonitrile

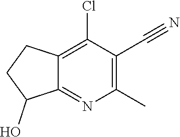

To a solution of 4-chloro-3-cyano-2-methyl-6,7-dihydro-5H-cyclopenta[b]pyridine 1-oxide (1.84 g, 8.82 mmol, ˜50% pure) in DCM (42.54 mL) was added trifluoroacetic anhydride (3.68 mL, 26.46 mmol) dropwise and the mixture was stirred at 25° C. for 20 hrs. The solvent was evaporated and the residue was taken up in MeOH. K₂CO₃ was added until basic pH and the suspension was stirred at 25° C. for 1 hr. The solvent was evaporated keeping the temperature under 40° C. The residue was then taken up in H₂O and extracted with DCM (3×). The combined organic layers were passed through a phase separator and evaporated to dryness. The material was purified by silica gel column chromatography using a 0-50% EtOAc/cyclohexane gradient eluent to afford the title compound (452 mg, 24%) as a pale yellow solid. ¹H NMR (400 MHz, DMSO-d₆) δ 5.76 (d, J=5.8 Hz, 1H), 4.99 (dt, J=7.6, 6.0 Hz, 1H), 2.93 (ddd, J=16.6, 8.9, 4.2 Hz, 1H), 2.72 (dt, J=16.4, 7.8 Hz, 1H), 2.48-2.38 (m, 1H), 2.46 (s, 3H), 1.85 (ddt, J=13.1, 9.0, 6.7 Hz, 1H). MS-ESI (m/z) calc'd for C₁₀H₁₀ClN₂O [M+H]⁺: 209.1. Found 209.0.

### Step 5: 7-Hydroxy-2,4-dimethyl-6,7-dihydro-5H-cyclopenta[b]pyridine-3-carbonitrile

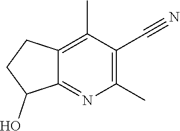

A solution of 4-chloro-7-hydroxy-2-methyl-6,7-dihydro-5H-cyclopenta[b]pyridine-3-carbonitrile (187.0 mg, 0.900 mmol), K₂CO₃ (247.75 mg, 1.79 mmol) and 2,4,6-trimethyl-1,3,5,2,4,6-trioxatriborinane (225.02 mg, 1.79 mmol) in 1,4-dioxane (6 mL) and H₂O (3 mL) was degassed with N₂ for 15 min. Then Pd(PPh₃)₄ (207.14 mg, 0.18 mmol) was added and the mixture was heated at 100° C. using microwave irradiation for 90 minutes. The reaction mixture was partitioned between H₂O and EtOAc, the phases were separated, and the aqueous layer was extracted with EtOAc (2×). The combined organic phases were washed with brine, passed through a phase separator, and evaporated to dryness. The residue was purified by reversed phase column chromatography on a 10 g column using a 0-70% MeCN/H₂O (0.1% HCOOH) gradient eluent to afford the title compound (41 mg, 24%) as an off-white solid. ¹H NMR (400 MHz, DMSO-d₆) δ 5.55 (d, J=5.7 Hz, 1H), 4.94 (dt, J=7.4, 5.8 Hz, 1H), 2.98-2.83 (m, 1H), 2.74-2.66 (m, 1H), 2.65 (s, 3H), 2.42-2.31 (m, 1H), 2.39 (s, 3H), 1.92-1.73 (m, 1H). MS-ESI (m/z) calc'd for C₁₁H₁₃N₂O [M+H]⁺: 189.1. Found 189.1.

### Step 6: N-(3-Cyano-2,4-dimethyl-6,7-dihydro-5H-cyclopenta[b]pyridin-7-yl)-2-nitro-N-(3-(oxazol-5-yl)-1-((2-(trimethylsilyl)ethoxy)methyl)-1H-indazol-5-yl)benzenesulfonamide

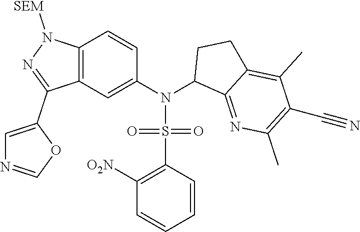 [SMILES: Cc1nc2c(c(C)c1C#N)CCC2N(c1ccc2c(c1)c(-c1cnco1)nn2COCC[Si](C)(C)C)S(=O)(=O)c1ccccc1[N+](=O)[O-]]

Diisopropyl azodicarboxylate (0.08 mL, 0.43 mmol) was added dropwise to a solution of 7-hydroxy-2,4-dimethyl-6,7-dihydro-5H-cyclopenta[b]pyridine-3-carbonitrile (40.0 mg, 0.21 mmol), 2-nitro-N-[3-(1,3-oxazol-5-yl)-1-(2-trimethylsilylethoxymethyl)indazol-5-yl]benzenesulfonamide (109.57 mg, 0.21 mmol) and triphenylphosphine (111.48 mg, 0.43 mmol) in THF (2.68 mL) and the mixture was stirred at 25° C. for 2 hrs. The reaction mixture was partitioned between H₂O and EtOAc, the phases were separated, and the aqueous layer was extracted with EtOAc (2×). The combined organic phases were washed with brine, dried over Na₂SO₄, filtered, and evaporated to dryness. The residue was purified by silica gel column chromatography using a 0-70% EtOAc/cyclohexane gradient eluent to afford the title compound (145 mg, 99%) as an orange solid. ¹H NMR (400 MHz, DMSO-d₆) δ 8.57 (s, 1H), 8.16 (d, J=7.9 Hz, 1H), 7.98-7.94 (m, 2H), 7.90-7.84 (m, 1H), 7.71 (d, J=9.0 Hz, 1H), 7.57 (d, J=1.9 Hz, 1H), 7.55 (s, 1H), 7.12 (dd, J=8.9, 1.9 Hz, 1H), 6.02 (t, J=8.0 Hz, 1H), 5.75 (s, 2H), 3.51 (t, J=7.9 Hz, 2H), 2.73-2.56 (m, 5H), 2.37-2.28 (m, 1H), 2.17 (s, 3H), 2.08-2.01 (m, 1H), 0.76 (t, J=8.0 Hz, 2H), −0.18 (s, 9H). MS-ESI (m/z) calc'd for C₃₃H₃₆N₇O₆SSi [M+H]⁺: 686.2. Found 686.3.

### Step 7: 2,4-Dimethyl-7-((3-(oxazol-5-yl)-1-((2-(trimethylsilyl)ethoxy)methyl)-1H-indazol-5-yl)amino)-6,7-dihydro-5H-cyclopenta[b]pyridine-3-carbonitrile

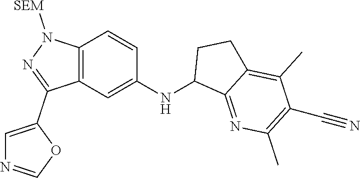 [SMILES: Cc1nc2c(c(C)c1C#N)CCC2Nc1ccc2c(c1)c(-c1cnco1)nn2COCC[Si](C)(C)C]

To a solution of N-(3-cyano-2,4-dimethyl-6,7-dihydro-5H-cyclopenta[b]pyridin-7-yl)-2-nitro-N-[3-(1,3-oxazol-5-yl)-1-(2-trimethylsilylethoxymethyl)indazol-5-yl]benzenesulfonamide (145.0 mg, 0.21 mmol) in DMF (2.2 mL) were added K₂CO₃ (116.88 mg, 0.85 mmol) and benzenethiol (0.06 mL, 0.63 mmol) and the mixture was stirred at 25° C. for 2 hrs. Water was added and the mixture was extracted with EtOAc (3×). The combined organic layers were dried over Na₂SO₄, filtered, and concentrated under reduced pressure. The residue was purified by SCX using a 2 g cartridge, washing with MeOH, and then eluting with a 2 M solution of NH₃ in MeOH to afford the title compound (100 mg, 94%), as a brown solid. ¹H NMR (400 MHz, DMSO-d₆) δ 8.51 (s, 1H), 7.73 (s, 1H), 7.56 (d, J=9.0 Hz, 1H), 7.20 (d, J=2.0 Hz, 1H), 7.09 (dd, J=9.1, 2.1 Hz, 1H), 6.02 (d, J=6.8 Hz, 1H), 5.71 (s, 2H), 5.03 (q, J=7.0 Hz, 1H), 3.54 (t, J=7.9 Hz, 2H), 3.07-2.94 (m, 1H), 2.85 (dt, J=15.9, 7.7 Hz, 1H), 2.74-2.65 (m, 1H), 2.63 (s, 3H), 2.45 (s, 3H), 1.92 (dq, J=15.4, 7.6 Hz, 1H), 0.81 (t, J=8.0 Hz, 2H), −0.10 (s, 9H). MS-ESI (m/z) calc'd for C₂₇H₃₃N₆O₂Si [M+H]⁺: 501.2. Found 501.3.

### Step 8: 2,4-Dimethyl-7-[[3-(1,3-oxazol-5-yl)-1H-indazol-5-yl]amino]-6,7-dihydro-5H-cyclopenta[b]pyridine-3-carbonitrile

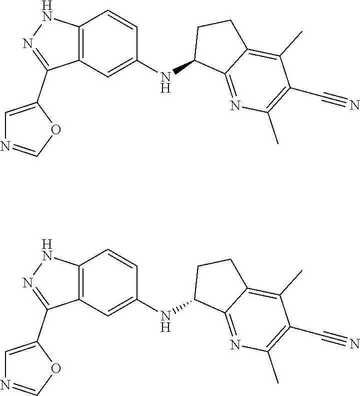 [SMILES: Cc1nc2c(c(C)c1C#N)CC[C@@H]2Nc1ccc2[nH]nc(-c3cnco3)c2c1 ; Cc1nc2c(c(C)c1C#N)CC[C@H]2Nc1ccc2[nH]nc(-c3cnco3)c2c1]

Prepared as described for 1-methoxy-5-((3-(oxazol-5-yl)-1H-indazol-5-yl)amino)-5,6,7,8-tetrahydronaphthalene-2-carbonitrile using 2,4-dimethyl-7-[[3-(1,3-oxazol-5-yl)-1-(2-trimethylsilylethoxymethyl)indazol-5-yl]amino]-6,7-dihydro-5H-cyclopenta[b]pyridine-3-carbonitrile in place of 1-methoxy-5-((3-(oxazol-5-yl)-1-((2-(trimethylsilyl)ethoxy)methyl)-1H-indazol-5-yl)amino)-5,6,7,8-tetrahydronaphthalene-2-carbonitrile to afford 2,4-dimethyl-7-[[3-(1,3-oxazol-5-yl)-1H-indazol-5-yl]amino]-6,7-dihydro-5H-cyclopenta[b]pyridine-3-carbonitrile (65 mg, 88%), which was subjected to chiral separation using Method GQ to afford 2,4-dimethyl-7-[[3-(1,3-oxazol-5-yl)-1H-indazol-5-yl]amino]-6,7-dihydro-5H-cyclopenta[b]pyridine-3-carbonitrile, enantiomer 1 (9 mg, 12%) as a white solid. ¹H NMR (400 MHz, DMSO-d₆) δ 13.41 (s, 1H), 8.51 (s, 1H), 7.82 (s, 1H), 7.66 (d, J=2.3 Hz, 1H), 7.56 (d, J=9.1 Hz, 1H), 7.17 (dd, J=9.0, 2.3 Hz, 1H), 5.97 (dd, J=7.3, 4.4 Hz, 1H), 3.15-3.02 (m, 1H), 2.99-2.88 (m, 1H), 2.80-2.68 (m, 1H), 2.53 (s, 3H), 2.24-2.12 (m, 1H). MS-ESI (m/z) calc'd for C₂₂H₁₉N₅O [M+H]⁺: 371.2 Found 371.2. A later eluting fraction was also isolated to afford 2,4-dimethyl-7-[[3-(1,3-oxazol-5-yl)-1H-indazol-5-yl]amino]-6,7-dihydro-5H-cyclopenta[b]pyridine-3-carbonitrile, enantiomer 2 (9 mg, 12%) as a white solid. ¹H NMR (400 MHz, DMSO-d₆) δ 13.08 (s, 1H), 8.47 (s, 1H), 7.65 (s, 1H), 7.37 (d, J=8.9 Hz, 1H), 7.16 (d, J=2.0 Hz, 1H), 7.02 (dd, J=9.0, 2.1 Hz, 1H), 5.90 (d, J=6.7 Hz, 1H), 4.99 (q, J=6.9 Hz, 1H), 3.00 (ddd, J=13.8, 8.8, 4.4 Hz, 1H), 2.85 (dt, J=16.1, 7.8 Hz, 1H), 2.74-2.64 (m, 1H), 2.63 (s, 3H), 2.45 (s, 3H), 1.99-1.86 (m, 1H). MS-ESI (m/z) calc'd for C₂₂H₁₉N₅O [M+H]⁺: 371.2. Found 371.2.

### Example 171: 2-Chloro-7-[[3-(1,3-oxazol-5-yl)-1H-indazol-5-yl]oxy]-6,7-dihydro-5H-cyclopenta[b]pyridine-3-carbonitrile, enantiomer 1 and 2

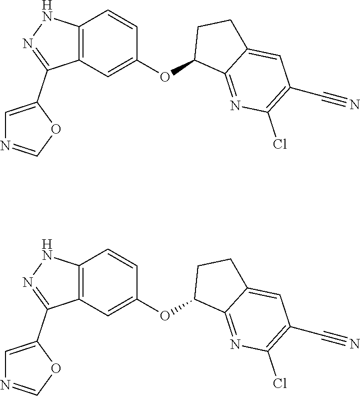 [SMILES: N#Cc1cc2c(nc1Cl)[C@@H](Oc1ccc3[nH]nc(-c4cnco4)c3c1)CC2 ; N#Cc1cc2c(nc1Cl)[C@H](Oc1ccc3[nH]nc(-c4cnco4)c3c1)CC2]

### Step 1: 2-Chloro-7-((3-(oxazol-5-yl)-1-((2-(trimethylsilyl)ethoxy)methyl)-1H-indazol-5-yl)oxy)-6,7-dihydro-5H-cyclopenta[b]pyridine-3-carbonitrile

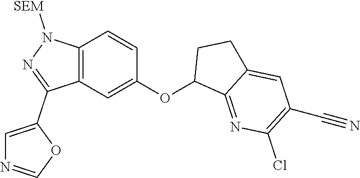 [SMILES: C[Si](C)(C)CCOCn1nc(-c2cnco2)c2cc(OC3CCc4cc(C#N)c(Cl)nc43)ccc21]

Diisopropyl azodicarboxylate (0.07 mL, 0.33 mmol) was added dropwise to a solution of 2-chloro-7-hydroxy-6,7-dihydro-5H-cyclopenta[b]pyridine-3-carbonitrile (70.0 mg, 0.28 mmol), 3-(1,3-oxazol-5-yl)-1-(2-trimethylsilylethoxymethyl)indazol-5-ol (91.79 mg, 0.28 mmol), and triphenylphosphine (145.28 mg, 0.550 mmol) in THF (5.723 mL) at 0° C. After 10 min, the mixture was brought to r.t. and stirred for 18 hrs. The reaction mixture was diluted with H₂O and EtOAc, the organic phase was separated, dried over Na₂SO₄, filtered, and concentrated under reduced pressure. The residue was purified by silica gel column chromatography using a 0-100% EtOAc/cyclohexane gradient eluent to afford the title compound (105 mg, 75%) as a beige solid. MS-ESI (m/z) calc'd for C₂₅H₂₇ClN₅O₃Si [M+H]⁺: 508.2. Found 508.3.

### Step 2: 2-Chloro-7-[[3-(1,3-oxazol-5-yl)-1H-indazol-5-yl]oxy]-6,7-dihydro-5H-cyclopenta[b]pyridine-3-carbonitrile

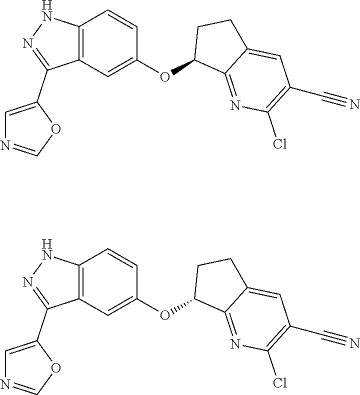 [SMILES: N#Cc1cc2c(nc1Cl)[C@@H](Oc1ccc3[nH]nc(-c4cnco4)c3c1)CC2 ; N#Cc1cc2c(nc1Cl)[C@H](Oc1ccc3[nH]nc(-c4cnco4)c3c1)CC2]

Prepared as described for 1-methoxy-5-((3-(oxazol-5-yl)-1H-indazol-5-yl)amino)-5,6,7,8-tetrahydronaphthalene-2-carbonitrile using 2-chloro-7-[3-(1,3-oxazol-5-yl)-1-(2-trimethylsilylethoxymethyl)indazol-5-yl]oxy-6,7-dihydro-5H-cyclopenta[b]pyridine-3-carbonitrile in place of 1-methoxy-5-((3-(oxazol-5-yl)-1-((2-(trimethylsilyl)ethoxy)methyl)-1H-indazol-5-yl)amino)-5,6,7,8-tetrahydronaphthalene-2-carbonitrile to afford 2-chloro-7-[[3-(1,3-oxazol-5-yl)-1H-indazol-5-yl]oxy]-6,7-dihydro-5H-cyclopenta[b]pyridine-3-carbonitrile (3.5 mg, 30%), which was subjected to chiral separation using Method GR to afford 2-chloro-7-[[3-(1,3-oxazol-5-yl)-1H-indazol-5-yl]oxy]-6,7-dihydro-5H-cyclopenta[b]pyridine-3-carbonitrile, enantiomer 1 (0.8 mg, 7%) as a white solid. ¹H NMR (400 MHz, MeOD) δ 8.37 (s, 1H), 8.23 (t, J=1.1 Hz, 1H), 7.80 (d, J=2.3 Hz, 1H), 7.71 (s, 1H), 7.53 (dd, J=9.0, 0.7 Hz, 1H), 7.24 (dd, J=9.0, 2.3 Hz, 1H), 5.84 (dd, J=7.1, 4.4 Hz, 1H), 3.26-3.15 (m, 1H), 3.09-2.98 (m, 1H), 2.83-2.70 (m, 1H), 2.37 (ddt, J=13.0, 9.0, 4.8 Hz, 1H). MS-ESI (m/z) calc'd for C₁₉H₁₂ClN₅O₂ [M+H]⁺: 378.1. Found 378.2. A later eluting fraction was also isolated to afford 2-chloro-7-[[3-(1,3-oxazol-5-yl)-1H-indazol-5-yl]oxy]-6,7-dihydro-5H-cyclopenta[b]pyridine-3-carbonitrile, enantiomer 2 (0.7 mg, 6%) as a white solid. ¹H NMR (400 MHz, MeOD) δ 8.36 (s, 1H), 8.26-8.19 (m, 1H), 7.80 (d, J=2.3 Hz, 1H), 7.71 (s, 1H), 7.53 (dd, J=9.0, 0.7 Hz, 1H), 7.24 (dd, J=9.1, 2.3 Hz, 1H), 5.83 (dd, J=7.1, 4.3 Hz, 1H), 3.27-3.15 (m, 1H), 3.04 (dddd, J=16.9, 8.6, 5.2, 1.1 Hz, 1H), 2.77 (dddd, J=14.2, 8.6, 7.1, 5.8 Hz, 1H), 2.37 (dddd, J=13.9, 8.6, 5.2, 4.3 Hz, 1H). MS-ESI (m/z) calc'd for C₁₉H₁₃ClN₅O₂ [M+H]⁺: 378.1. Found 378.1.

### Example 172: 6,6-Dimethyl-7-[[3-(1,3-oxazol-5-yl)-1H-indazol-5-yl]amino]-5,7-dihydrocyclopenta[b]pyridine-3-carbonitrile, enantiomer 1 and 2

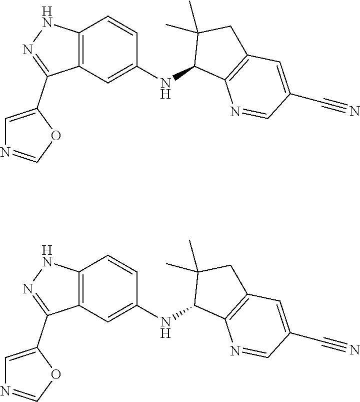 [SMILES: CC1(C)Cc2cc(C#N)cnc2[C@H]1Nc1ccc2[nH]nc(-c3cnco3)c2c1 ; CC1(C)Cc2cc(C#N)cnc2[C@@H]1Nc1ccc2[nH]nc(-c3cnco3)c2c1]

### Step 1: 3-Bromo-5,6-dihydro-7H-cyclopenta[b]pyridin-7-one

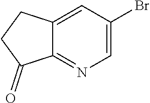 [SMILES: O=C1CCc2cc(Br)cnc21]

A solution of 3-bromo-6,7-dihydro-5H-cyclopenta[b]pyridin-7-ol (260.0 mg, 1.21 mmol) in DCM (10 mL) was treated with Dess-Martin periodinane (515.17 mg, 1.21 mmol) and stirred at r.t. for 2 hrs. The reaction was diluted with DCM and quenched by addition of 2 mL of saturated aqueous NaHCO₃. After stirring at r.t. for 5 minutes, the phases were separated and the aqueous layer was extracted with DCM (1×). The combined organic phases were washed with saturated aqueous NaHCO₃, passed through a phase separator and evaporated to dryness. The residue was purified by silica gel column chromatography using a 0-100% EtOAc/cyclohexane gradient eluent to afford the title compound (207 mg, 80%) as a beige solid. ¹H NMR (400 MHz, DMSO-d₆) δ 8.87-8.77 (m, 1H), 8.45-8.36 (m, 1H), 3.16-3.04 (m, 2H), 2.73-2.61 (m, 2H). MS-ESI (m/z) calc'd for C₈H₇BrNO [M+H]⁺: 212.0, 214.0. Found 211.9, 213.9.

### Step 2: 3-Bromo-6,6-dimethyl-5,6-dihydro-7H-cyclopenta[b]pyridin-7-one

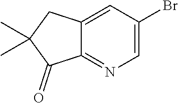

Iodomethane (0.09 mL, 1.41 mmol) was added dropwise to a solution of 3-bromo-5,6-dihydro-7H-cyclopenta[b]pyridin-7-one (150.0 mg, 0.71 mmol) and potassium tert-butoxide (158.76 mg, 1.41 mmol) in THF (7 mL) and the mixture was stirred under an N₂ atmosphere at r.t. for 2 hrs. Saturated aqueous NH₄Cl (50 mL) and EtOAc (50 mL) were added and the organic phase was separated, dried over Na₂SO₄, filtered, and concentrated under reduced pressure. The residue was purified by silica gel column chromatography using a 0-100% EtOAc/cyclohexane gradient eluent to afford the title compound (105 mg, 62%) as a dark solid. ¹H NMR (400 MHz, MeOD) δ 8.79 (dt, J=2.0, 0.9 Hz, 1H), 8.28 (dt, J=2.0, 1.0 Hz, 1H), 3.06 (t, J=0.9 Hz, 2H), 1.24 (s, 6H). MS-ESI (m/z) calc'd for C₁₀H₁₁BrNO [M+H]⁺: 240.0, 242.0. Found 240.0; 242.0.

### Step 3: 3-Bromo-6,6-dimethyl-6,7-dihydro-5H-cyclopenta[b]pyridin-7-ol

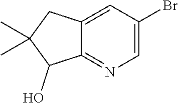

To a solution of 3-bromo-6,6-dimethyl-5H-cyclopenta[b]pyridin-7-one (113.0 mg, 0.34 mmol) in MeOH (2.5 mL) was added sodium borohydride (12.82 mg, 0.34 mmol) at 25° C. The resulting mixture was stirred for 1 hr. The reaction mixture was then quenched with saturated aqueous NaHCO₃ (50 mL) and diluted with DCM (50 mL). The organic layer was washed with brine, dried over Na₂SO₄, filtered, and concentrated to give material which was purified by column chromatography on a C18 cartridge using a 0-80% MeCN/H₂O (0.1% HCOOH) gradient eluent to afford the title compound (54 mg, 66%) as a white solid. ¹H NMR (400 MHz, DMSO-d₆) δ 8.49-8.40 (m, 1H), 7.85 (dt, J=2.2, 1.1 Hz, 1H), 5.42 (d, J=6.1 Hz, 1H), 4.37 (d, J=6.0 Hz, 1H), 2.73-2.66 (m, 1H), 2.58 (d, J=16.0 Hz, 1H), 1.08 (s, 3H), 0.92 (s, 3H). MS-ESI (m/z) calc'd for C₁₀H₁₃BrNO [M+H]⁺: 242.0, 244.0. Found 242.0, 244.0.

### Step 4: N-(3-Bromo-6,6-dimethyl-5,7-dihydrocyclopenta[b]pyridin-7-yl)-2-nitro-N-[3-(1,3-oxazol-5-yl)-1-(2-trimethylsilylethoxymethyl)indazol-5-yl]benzenesulfonamide

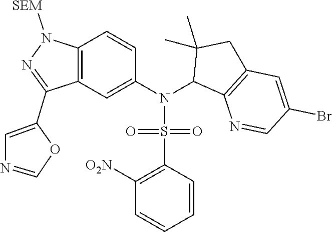 [SMILES: CC1(C)Cc2cc(Br)cnc2C1N(c1ccc2c(c1)c(-c1cnco1)nn2COCC[Si](C)(C)C)S(=O)(=O)c1ccccc1[N+](=O)[O-]]

Diisopropyl azodicarboxylate (0.05 mL, 0.27 mmol) was added dropwise to a solution of 3-bromo-6,6-dimethyl-6,7-dihydro-5H-cyclopenta[b]pyridin-7-ol (54.0 mg, 0.22 mmol), 2-nitro-N-[3-(1,3-oxazol-5-yl)-1-(2-trimethylsilylethoxymethyl)indazol-5-yl]benzenesulfonamide (115.0 mg, 0.22 mmol) and triphenylphosphine (117.0 mg, 0.45 mmol) in THF (3 mL) and the mixture was stirred at r.t. for 2 hrs. The reaction mixture was diluted with H₂O (50 mL) and EtOAc (3×50 mL). The organic phase was separated, dried over Na₂SO₄, filtered, and concentrated under reduced pressure. The residue was purified by silica gel column chromatography using a 0-70% EtOAc/cyclohexane gradient eluent to afford the title compound (196 mg, 72%), as a yellow oil. MS-ESI (m/z) calc'd for C₃₂H₃₆BrN₆O₆SSi [M+H]⁺: 739.1, 741.1. Found 739.3, 741.3.

### Step 5: N-(3-Cyano-6,6-dimethyl-6,7-dihydro-5H-cyclopenta[b]pyridin-7-yl)-2-nitro-N-(3-(oxazol-5-yl)-1-((2-(trimethylsilyl)ethoxy)methyl)-1H-indazol-5-yl)benzenesulfonamide

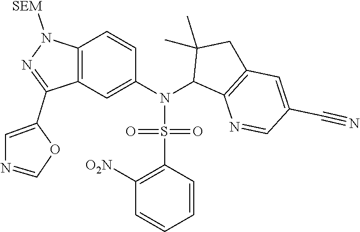 [SMILES: CC1(C)Cc2cc(C#N)cnc2C1N(c1ccc2c(c1)c(-c1cnco1)nn2COCC[Si](C)(C)C)S(=O)(=O)c1ccccc1[N+](=O)[O-]]

0.1 N Aqueous potassium hexacyanoferrate (II) (2.23 mL, 0.22 mmol), N-(3-bromo-6,6-dimethyl-5,7-dihydrocyclopenta[b]pyridin-7-yl)-2-nitro-N-[3-(1,3-oxazol-5-yl)-1-(2-trimethylsilylethoxymethyl)indazol-5-yl]benzenesulfonamide (165.0 mg, 0.22 mmol) and KOAc (21.89 mg, 0.22 mmol) were dissolved in a mixture of 1,4-dioxane (1.786 mL) and H₂O (0.595 mL) in a sealed microwave vial. The mixture was degassed with N₂ for 15 minutes. Then XPhos (18.49 mg, 0.040 mmol) and XPhos Pd G3 (18.88 mg, 0.020 mmol) were added and the mixture was stirred at 110° C. for 2 hrs. The reaction was brought to r.t. and then diluted with H₂O (50 mL) and EtOAc (50 mL). The organic phase was separated, dried over Na₂SO₄, filtered, and concentrated under reduced pressure. The residue was purified by silica gel column chromatography using a 0-100% EtOAc/cyclohexane gradient eluent to afford the title compound (127 mg, 83%) as a yellow solid. MS-ESI (m/z) calc'd for C₃₃H₃₆N₇O₆SSi [M+H]⁺: 686.2. Found 686.3.

### Step 6: 6,6-Dimethyl-7-((3-(oxazol-5-yl)-1-((2-(trimethylsilyl)ethoxy)methyl)-1H-indazol-5-yl)amino)-6,7-dihydro-5H-cyclopenta[b]pyridine-3-carbonitrile

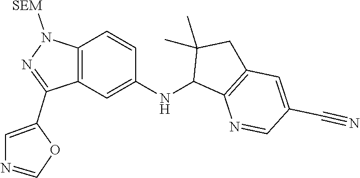 [SMILES: CC1(C)Cc2cc(C#N)cnc2C1Nc1ccc2c(c1)c(-c1cnco1)nn2COCC[Si](C)(C)C]

To a solution of N-(3-Cyano-6,6-dimethyl-6,7-dihydro-5H-cyclopenta[b]pyridin-7-yl)-2-nitro-N-(3-(oxazol-5-yl)-1-((2-(trimethylsilyl)ethoxy)methyl)-1H-indazol-5-yl)benzenesulfonamide (127.0 mg, 0.19 mmol) in DMF (1.7 mL) were added K₂CO₃ (102.37 mg, 0.74 mmol) and benzenethiol (56.83 uL, 0.56 mmol) and the mixture was stirred at 25° C. for 1 hr. Water (50 mL) was added and the mixture was extracted with EtOAc (3×50 mL). The combined organic layers were evaporated to give a yellow oil which was passed through an SCX cartridge to afford the title compound (52 mg, 56%) as a yellow oil. ¹H NMR (400 MHz, DMSO-d₆) δ 12.91 (1H, s) 8.83 (1H, d, J=1.76 Hz) 8.15 (1H, d, J=1.76 Hz) 7.29 (1H, s) 6.64 (1H, s) 5.21 (1H, d, J=5.50 Hz) 4.59-4.76 (1H, m) 2.90 (2H, br. s.) 2.19-2.33 (4H, m) 1.79-2.05 (3H, m). MS-ESI (m/z) calc'd for C₂₇H₃₃N₆O₂Si [M+H]⁺: 501.2. Found 501.3.

### Step 7: 6,6-Dimethyl-7-[[3-(1,3-oxazol-5-yl)-1H-indazol-5-yl]amino]-5,7-dihydrocyclopenta[b]pyridine-3-carbonitrile, enantiomer 1 and 2

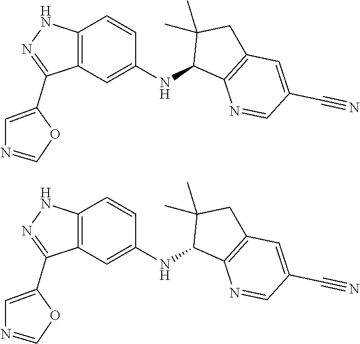

Prepared as described for 1-methoxy-5-((3-(oxazol-5-yl)-1H-indazol-5-yl)amino)-5,6,7,8-tetrahydronaphthalene-2-carbonitrile using 6,6-dimethyl-7-[[3-(1,3-oxazol-5-yl)-1-(2-trimethylsilylethoxymethyl)indazol-5-yl]amino]-5,7-dihydrocyclopenta[b]pyridine-3-carbonitrile in place of 1-methoxy-5-((3-(oxazol-5-yl)-1-((2-(trimethylsilyl)ethoxy)methyl)-1H-indazol-5-yl)amino)-5,6,7,8-tetrahydronaphthalene-2-carbonitrile to afford 6,6-dimethyl-7-[[3-(1,3-oxazol-5-yl)-1H-indazol-5-yl]amino]-5,7-dihydrocyclopenta[b]pyridine-3-carbonitrile, which was subjected to chiral separation using Method GS to afford 6,6-dimethyl-7-[[3-(1,3-oxazol-5-yl)-1H-indazol-5-yl]amino]-5,7-dihydrocyclopenta[b]pyridine-3-carbonitrile, enantiomer 1 (1 mg, 3%) as a yellow solid ¹H NMR (400 MHz, MeOD) δ 8.67 (s, 1H), 8.32 (s, 1H), 8.03-7.92 (m, 1H), 7.58 (s, 1H), 7.40 (dd, J=9.0, 0.7 Hz, 1H), 7.31 (d, J=2.1 Hz, 1H), 7.18 (dd, J=9.0, 2.2 Hz, 1H), 4.97 (s, 1H), 3.00-2.81 (m, 2H), 1.42 (s, 3H), 1.03 (s, 3H). MS-ESI (m/z) calc'd for C₂₁H₁₈N₆O [M+H]⁺: 371.2 Found 371.2. A later eluting fraction was also isolated to afford 6,6-dimethyl-7-[[3-(1,3-oxazol-5-yl)-1H-indazol-5-yl]amino]-5,7-dihydrocyclopenta[b]pyridine-3-carbonitrile, enantiomer 2 (0.7 mg, 2%) as a yellow solid. ¹H NMR (400 MHz, MeOD) δ 8.67 (d, J=1.8 Hz, 1H), 8.32 (s, 1H), 7.99 (d, J=1.9 Hz, 1H), 7.58 (s, 1H), 7.40 (d, J=9.0 Hz, 1H), 7.31 (d, J=2.1 Hz, 1H), 7.18 (dd, J=9.0, 2.2 Hz, 1H), 4.97 (s, 1H), 3.00-2.79 (m, 2H), 1.42 (s, 3H), 1.03 (s, 3H). MS-ESI (m/z) calc'd for C₂₁H₁₉N₆O [M+H]⁺: 371.2. Found 371.2.

### Example 173: cis-6-Methyl-5-((3-(oxazol-5-yl)-1H-indazol-5-yl)amino)-5,6,7,8-tetrahydronaphthalene-2-carbonitrile, enantiomer 1 and 2 and Example 174: trans-6-Methyl-5-((3-(oxazol-5-yl)-1H-indazol-5-yl)amino)-5,6,7,8-tetrahydronaphthalene-2-carbonitrile, enantiomer 1 and 2

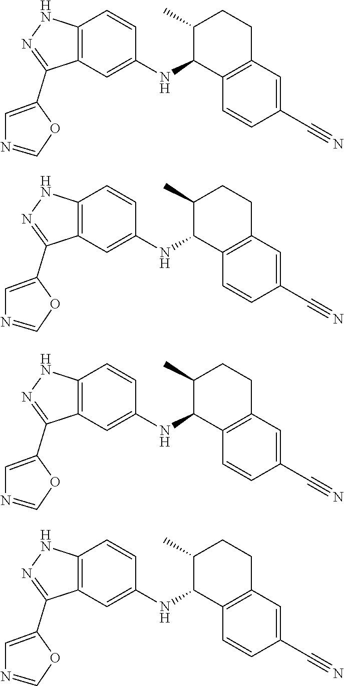

### Step 1: N-(6-Cyano-2-methyl-1,2,3,4-tetrahydronaphthalen-1-yl)-2-nitro-N-(3-(oxazol-5-yl)-1-((2-(trimethylsilyl)ethoxy)methyl)-1H-indazol-5-yl)benzenesulfonamide

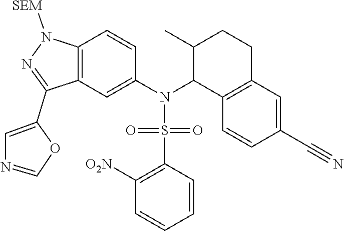 [SMILES: CC1CCc2cc(C#N)ccc2C1N(c1ccc2c(c1)c(-c1cnco1)nn2COCC[Si](C)(C)C)S(=O)(=O)c1ccccc1[N+](=O)[O-]]

Diisopropyl azodicarboxylate was added (0.07 mL, 0.33 mmol) to a solution of 5-hydroxy-6-methyl-5,6,7,8-tetrahydronaphthalene-2-carbonitrile (52.0 mg, 0.28 mmol), 2-nitro-N-[3-(1,3-oxazol-5-yl)-1-(2-trimethylsilylethoxymethyl)indazol-5-yl]benzenesulfonamide (143.19 mg, 0.28 mmol), and triphenylphosphine (145.69 mg, 0.56 mmol) in THF (3.12 mL) and the mixture was stirred at r.t. for 18 hrs. The reaction mixture was diluted with H₂O (50 mL) and extracted with EtOAc (3×50 mL). The organic phase was separated, dried over Na₂SO₄, filtered, and concentrated under reduced pressure. The residue was purified by silica gel column chromatography using a 0-100% EtOAc/cyclohexane gradient eluent to afford the title compound (190 mg, 99%) as a yellow solid. MS-ESI (m/z) calc'd for C₃₄H₃₇N₆O₆SSi [M+H]⁺: 685.2. Found 685.3.

### Step 2: 6-Methyl-5-((3-(oxazol-5-yl)-1-((2-(trimethylsilyl)ethoxy)methyl)-1H-indazol-5-yl)amino)-5,6,7,8-tetrahydronaphthalene-2-carbonitrile

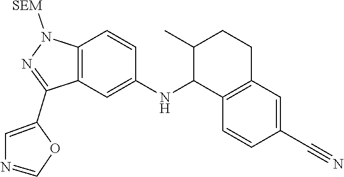 [SMILES: CC1CCc2cc(C#N)ccc2C1Nc1ccc2c(c1)c(-c1cnco1)nn2COCC[Si](C)(C)C]

To a solution of N-(6-cyano-2-methyl-1,2,3,4-tetrahydronaphthalen-1′-yl)-2-nitro-N-[3-(1,3-oxazol-5-yl)-1-(2-trimethylsilylethoxymethyl)indazol-5-yl]benzenesulfonamide (190.0 mg, 0.28 mmol) in DMF (2.5 mL) was added K₂CO₃ (153.38 mg, 1.11 mmol) and benzenethiol (0.09 mL, 0.83 mmol) and the mixture was stirred at 25° C. for 1 hr. Water (50 mL) was added and the mixture was extracted with EtOAc (3×50 mL). The combined organic layers were evaporated to give a yellow oil which was passed through an SCX cartridge to afford the title compound (109 mg, 79%) as a yellow oil. MS-ESI (m/z) calc'd for C₂₈H₃₄N₅O₂Si [M+H]⁺: 500.2. Found 500.3.

### Step 3: cis-6-Methyl-5-((3-(oxazol-5-yl)-1H-indazol-5-yl)amino)-5,6,7,8-tetrahydronaphthalene-2-carbonitrile, enantiomer 1 and 2, and trans-6-Methyl-5-((3-(oxazol-5-yl)-1H-indazol-5-yl)amino)-5,6,7,8-tetrahydronaphthalene-2-carbonitrile, enantiomer 1 and 2

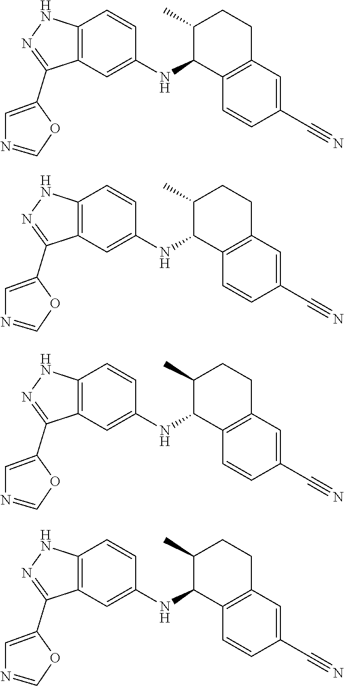

Prepared as described for 1-methoxy-5-((3-(oxazol-5-yl)-1H-indazol-5-yl)amino)-5,6,7,8-tetrahydronaphthalene-2-carbonitrile using 6-methyl-5-[[3-(1,3-oxazol-5-yl)-1-(2-trimethylsilylethoxymethyl)indazol-5-yl]amino]-5,6,7,8-tetrahydronaphthalene-2-carbonitrile in place of 1-methoxy-5-((3-(oxazol-5-yl)-1-((2-(trimethylsilyl)ethoxy)methyl)-1H-indazol-5-yl)amino)-5,6,7,8-tetrahydronaphthalene-2-carbonitrile to afford a mixture of four isomers, which was subjected to chiral separation using Method GT to afford the first eluting isomer, trans-6-methyl-5-[[3-(1,3-oxazol-5-yl)-1H-indazol-5-yl]amino]-5,6,7,8-tetrahydronaphthalene-2-carbonitrile, enantiomer 1 (9.9 mg, 11%) as a white solid ¹H NMR (400 MHz, CDCl₃) δ 10.08 (s, 1H), 7.99 (s, 1H), 7.54 (d, J=8.1 Hz, 1H), 7.49 (s, 1H), 7.45 (d, J=1.6 Hz, 1H), 7.40 (dd, J=8.1, 1.7 Hz, 1H), 7.37 (d, J=8.9 Hz, 1H), 7.03 (d, J=2.2 Hz, 1H), 6.88 (dd, J=8.9, 2.2 Hz, 1H), 4.28 (t, J=7.5 Hz, 1H), 3.99 (d, J=8.3 Hz, 1H), 2.90 (t, J=6.5 Hz, 2H), 2.21-2.11 (m, 1H), 2.10-1.99 (m, 1H), 1.72 (ddt, J=13.9, 9.0, 7.1 Hz, 1H), 1.15 (d, J=6.8 Hz, 3H). MS-ESI (m/z) calc'd for C₂₂H₂₀N₅O [M+H]⁺: 370.2. Found 370.1. A second eluting isomer was isolated to give cis-6-methyl-5-[[3-(1,3-oxazol-5-yl)-1H-indazol-5-yl]amino]-5,6,7,8-tetrahydronaphthalene-2-carbonitrile, enantiomer 1 (14 mg, 17%) as a white solid. ¹H NMR (400 MHz, CDCl₃) δ 10.06 (s, 1H), 8.01 (s, 1H), 7.55 (s, 1H), 7.50 (d, J=8.0 Hz, 1H), 7.47-7.43 (m, 1H), 7.42-7.35 (m, 2H), 7.19 (d, J=2.2 Hz, 1H), 6.94 (dd, J=8.9, 2.2 Hz, 1H), 4.74 (dd, J=9.7, 4.5 Hz, 1H), 3.74 (d, J=9.7 Hz, 1H), 3.04-2.74 (m, 2H), 2.44-2.31 (m, 1H), 1.96 (dtd, J=13.5, 6.6, 3.2 Hz, 1H), 1.87-1.74 (m, 1H), 1.06 (d, J=6.8 Hz, 3H) MS-ESI (m/z) calc'd for C₂₂H₂₀N₅O [M+H]⁺: 370.2. Found 370.2. The residue was again subjected to semipreparative chiral HPLC, using Method GU to afford trans-6-methyl-5-[[3-(1,3-oxazol-5-yl)-1H-indazol-5-yl]amino]-5,6,7,8-tetrahydronaphthalene-2-carbonitrile, enantiomer 2 (7.6 mg, 9%) as a white solid. ¹H NMR (400 MHz, CDCl₃) δ 10.02 (s, 1H), 7.99 (s, 1H), 7.54 (d, J=8.0 Hz, 1H), 7.49 (s, 1H), 7.45 (d, J=1.6 Hz, 1H), 7.40 (dd, J=8.0, 1.7 Hz, 1H), 7.37 (d, J=8.9 Hz, 1H), 7.03 (d, J=2.2 Hz, 1H), 6.88 (dd, J=8.9, 2.2 Hz, 1H), 4.28 (t, J=7.4 Hz, 1H), 3.98 (d, J=8.3 Hz, 1H), 2.90 (t, J=6.5 Hz, 2H), 2.22-2.11 (m, 1H), 2.10-1.97 (m, 1H), 1.72 (ddt, J=13.9, 8.9, 7.1 Hz, 1H), 1.15 (d, J=6.7 Hz, 3H). MS-ESI (m/z) calc'd for C₂₂H₂₀N₅O [M+H]⁺: 370.2. Found 370.2. A second eluting isomer was isolated to give cis-6-methyl-5-[[3-(1,3-oxazol-5-yl)-1H-indazol-5-yl]amino]-5,6,7,8-tetrahydronaphthalene-2-carbonitrile, enantiomer 2 (13.4 mg, 17%) as a white solid. ¹H NMR (400 MHz, CDCl₃) δ 10.07 (s, 1H), 8.02 (s, 1H), 7.55 (s, 1H), 7.50 (d, J=8.0 Hz, 1H), 7.45 (d, J=1.7 Hz, 1H), 7.42-7.36 (m, 2H), 7.19 (d, J=2.1 Hz, 1H), 6.94 (dd, J=8.9, 2.2 Hz, 1H), 4.74 (dd, J=9.7, 4.5 Hz, 1H), 3.74 (d, J=9.7 Hz, 1H), 3.02-2.77 (m, 2H), 2.46-2.29 (m, 1H), 1.96 (dtd, J=13.4, 6.6, 3.3 Hz, 1H), 1.80 (dtd, J=13.9, 7.8, 6.1 Hz, 1H), 1.06 (d, J=6.9 Hz, 3H). MS-ESI (m/z) calc'd for C₂₂H₂₀N₅O [M+H]⁺: 370.2. Found 370.1.

### Example 175: cis-6-Methyl-5-[[3-(1,3-oxazol-5-yl)-1H-indazol-5-yl]oxy]-5,6,7,8-tetrahydronaphthalene-2-carbonitrile, enantiomer 1 and 2 and Example 176: trans-6-Methyl-5-[[3-(1,3-oxazol-5-yl)-1H-indazol-5-yl]oxy]-5,6,7,8-tetrahydronaphthalene-2-carbonitrile, enantiomer 1 and 2

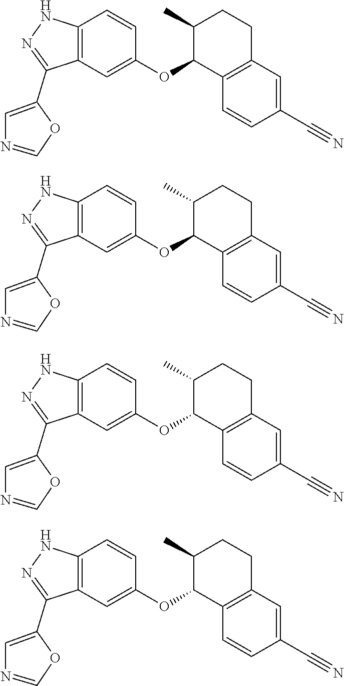

### Step 1: 6-Methyl-5-oxo-5,6,7,8-tetrahydronaphthalene-2-carbonitrile

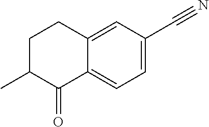

To a solution of 6-cyano-1-tetralone (2.43 g, 14.18 mmol) and potassium tert-butoxide (1.75 g, 15.59 mmol) in THF (40 mL) was added iodomethane (0.88 mL, 14.18 mmol) dropwise and the mixture was stirred under an N₂ atmosphere at r.t. for 1 hr. Then the reaction was diluted with EtOAc and washed with saturated aqueous NH₄Cl. The organic phase was separated and concentrated under reduced pressure to give a residue that was purified by reversed phase column chromatography using a 2-80% MeCN/H₂O (0.1% formic acid) gradient eluent to afford material of insufficient purity. This was further purified by silica gel column chromatography using a 0-50% EtOAc/cyclohexane gradient eluent to afford the title compound (1.0 g, 38%) as a white solid. MS-ESI (m/z) calc'd for C₁₂H₁₂NO [M+H]⁺: 186.1. Found 186.0.

### Step 2: 5-Hydroxy-6-methyl-5,6,7,8-tetrahydronaphthalene-2-carbonitrile

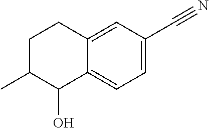

To a solution of 6-methyl-5-oxo-7,8-dihydro-6H-naphthalene-2-carbonitrile (1.0 g, 5.4 mmol) in MeOH (33.33 mL) was added sodium borohydride (204.24 mg, 5.4 mmol) at 25° C. The resulting mixture was stirred for 1 hr. The reaction was concentrated under reduced pressure and then saturated aqueous NaHCO₃ (100 mL) and DCM (100 mL) were added. The organic layer was washed with brine, dried over Na₂SO₄, filtered and concentrated. The residue was purified by silica gel column chromatography using a 2-100% MeCN/H₂O (0.1% formic acid) to afford the title compound (171 mg, 17%) as a colorless oil that was a mixture of two isomers. Isomer 1: ¹H NMR (400 MHz, DMSO-d₆) δ 7.65 (d, J=8.1 Hz, 1H), 7.61-7.58 (m, 1H), 7.53 (d, J=1.6 Hz, 1H), 5.46 (d, J=7.2 Hz, 1H), 4.11 (t, J=8.0 Hz, 1H), 2.81-2.74 (m, 2H), 1.92-1.80 (m, 1H), 1.73-1.66 (m, 1H), 1.53-1.40 (m, 1H), 1.06 (d, J=6.5 Hz, 3H). Isomer 2: ¹H NMR (400 MHz, DMSO-d₆) δ 7.60-7.58 (m, 1H), 7.57-7.55 (m, 1H), 7.49 (d, J=7.9 Hz, 1H), 5.18 (d, J=5.9 Hz, 1H), 4.45 (t, J=4.8 Hz, 1H), 2.87-2.63 (m, 2H), 1.85-1.81 (m, 1H), 1.73-1.57 (m, 2H), 0.96 (d, J=6.8 Hz, 3H). MS-ESI (m/z) calc'd for C₁₂H₁₄NO [M+H]⁺: 188.1. Found 188.1.

### Step 3: 6-Methyl-5-((3-(oxazol-5-yl)-1-((2-(trimethylsilyl)ethoxy)methyl)-1H-indazol-5-yl)oxy)-5,6,7,8-tetrahydronaphthalene-2-carbonitrile

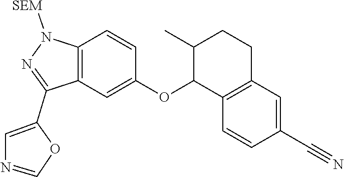 [SMILES: CC1CCc2cc(C#N)ccc2C1Oc1ccc2c(c1)c(-c1cnco1)nn2COCC[Si](C)(C)C]

Diisopropyl azodicarboxylate (0.11 mL, 0.54 mmol) was added dropwise to a solution of 5-hydroxy-6-methyl-5,6,7,8-tetrahydronaphthalene-2-carbonitrile (85.0 mg, 0.45 mmol), 3-(1,3-oxazol-5-yl)-1-(2-trimethylsilylethoxymethyl)indazol-5-ol (150.46 mg, 0.45 mmol) and triphenylphosphine (238.14 mg, 0.91 mmol) in THF (5.1 mL) and the mixture was stirred at r.t. for 18 hrs. The reaction mixture was diluted with H₂O (50 mL) and EtOAc (3×50 mL). The organic phase was separated, dried over Na₂SO₄, filtered, and concentrated under reduced pressure. The residue was purified by silica gel column chromatography using a 0-100% EtOAc/cyclohexane gradient eluent to afford the title compound (176 mg, 77%) as a pale yellow solid. MS-ESI (m/z) calc'd for C₂₈H₃₃N₄O₃Si [M+H]⁺: 501.2. Found 501.2.

### Step 4: cis-6-Methyl-5-[[3-(1,3-oxazol-5-yl)-1H-indazol-5-yl]oxy]-5,6,7,8-tetrahydronaphthalene-2-carbonitrile, enantiomer 1 and 2 and trans-6-Methyl-5-[[3-(1,3-oxazol-5-yl)-1H-indazol-5-yl]oxy]-5,6,7,8-tetrahydronaphthalene-2-carbonitrile, enantiomer 1 and 2

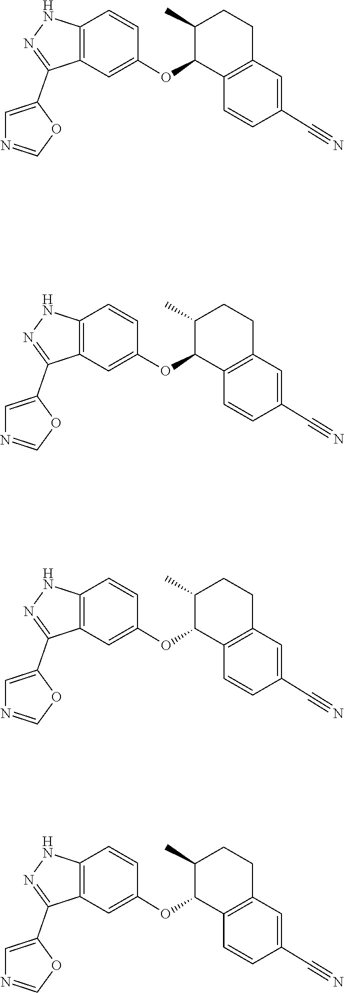 [SMILES: C[C@H]1CCc2cc(C#N)ccc2[C@H]1Oc1ccc2[nH]nc(-c3cnco3)c2c1 ; C[C@@H]1CCc2cc(C#N)ccc2[C@H]1Oc1ccc2[nH]nc(-c3cnco3)c2c1 ; C[C@@H]1CCc2cc(C#N)ccc2[C@@H]1Oc1ccc2[nH]nc(-c3cnco3)c2c1 ; C[C@H]1CCc2cc(C#N)ccc2[C@@H]1Oc1ccc2[nH]nc(-c3cnco3)c2c1]

Prepared as described for 1-methoxy-5-((3-(oxazol-5-yl)-1H-indazol-5-yl)amino)-5,6,7,8-tetrahydronaphthalene-2-carbonitrile using 6-methyl-5-[3-(1,3-oxazol-5-yl)-1-(2-trimethylsilylethoxymethyl)indazol-5-yl]oxy-5,6,7,8-tetrahydronaphthalene-2-carbonitrile in place of 1-methoxy-5-((3-(oxazol-5-yl)-1-((2-(trimethylsilyl)ethoxy)methyl)-1H-indazol-5-yl)amino)-5,6,7,8-tetrahydronaphthalene-2-carbonitrile to afford a mixture of four isomers, which was subjected to chiral separation using Method HB to afford the first eluted isomer cis-6-methyl-5-[[3-(1,3-oxazol-5-yl)-1H-indazol-5-yl]oxy]-5,6,7,8-tetrahydronaphthalene-2-carbonitrile, enantiomer 1 (1.5 mg, 1%) as a white solid. ¹H NMR (400 MHz, MeOD) δ 8.34 (s, 1H), 7.65 (s, 1H), 7.59 (d, J=2.2 Hz, 1H), 7.56 (d, J=1.4 Hz, 1H), 7.53 (dd, J=9.1, 0.7 Hz, 1H), 7.44-7.38 (m, 1H), 7.35 (d, J=8.0 Hz, 1H), 7.19 (dd, J=9.1, 2.3 Hz, 1H), 5.50 (d, J=3.8 Hz, 1H), 3.04 (dt, J=17.6, 6.1 Hz, 1H), 2.96-2.83 (m, 1H), 2.42-2.29 (m, 1H), 2.08-1.90 (m, 2H), 1.17 (d, J=6.9 Hz, 3H). MS-ESI (m/z) calc'd for C₂₂H₁₉N₄O₂ [M+H]⁺: 371.1. Found 371.2. The second eluting isomer was isolated, trans-6-methyl-5-[[3-(1,3-oxazol-5-yl)-1H-indazol-5-yl]oxy]-5,6,7,8-tetrahydronaphthalene-2-carbonitrile, enantiomer 1 (1.1 mg, 1%) as a white solid. ¹H NMR (400 MHz, MeOD) δ 8.35 (s, 1H), 7.65 (s, 1H), 7.60-7.55 (m, 3H), 7.50 (d, J=1.1 Hz, 2H), 7.26 (dd, J=9.1, 2.3 Hz, 1H), 5.28 (d, J=6.8 Hz, 1H), 2.97 (t, J=6.6 Hz, 2H), 2.41-2.27 (m, 1H), 2.21-2.11 (m, 1H), 1.83-1.70 (m, 1H), 1.13 (d, J=6.8 Hz, 3H). MS-ESI (m/z) calc'd for C₂₂H₁₉N₄O₂ [M+H]⁺: 371.1. Found 371.2. The third eluting isomer was cis-6-methyl-5-[[3-(1,3-oxazol-5-yl)-1H-indazol-5-yl]amino]-5,6,7,8-tetrahydronaphthalene-2-carbonitrile, enantiomer 2 (2.5 mg, 2%) as a white solid ¹H NMR (400 MHz, MeOD) δ 8.35 (s, 1H), 7.65 (s, 1H), 7.59 (d, J=2.3 Hz, 1H), 7.56 (d, J=1.6 Hz, 1H), 7.53 (d, J=9.0 Hz, 1H), 7.41 (dd, J=8.0, 1.7 Hz, 1H), 7.35 (d, J=8.0 Hz, 1H), 7.19 (dd, J=9.1, 2.3 Hz, 1H), 5.50 (d, J=3.8 Hz, 1H), 3.04 (dt, J=17.6, 6.1 Hz, 1H), 2.97-2.84 (m, 1H), 2.42-2.28 (m, 1H), 2.09-1.88 (m, 2H), 1.17 (d, J=6.8 Hz, 3H). MS-ESI (m/z) calc'd for C₂₂H₁₉N₄O₂ [M+H]⁺: 371.1. Found 371.2. The last eluting isomer was isolated, trans-6-methyl-5-[[3-(1,3-oxazol-5-yl)-1H-indazol-5-yl]amino]-5,6,7,8-tetrahydronaphthalene-2-carbonitrile, enantiomer 2 (1.1 mg, 1%) as a white solid. ¹H NMR (400 MHz, MeOD) δ 8.33 (s, 1H), 7.63 (s, 1H), 7.59-7.53 (m, 3H), 7.48 (d, J=1.2 Hz, 2H), 7.24 (dd, J=9.0, 2.4 Hz, 1H), 5.26 (d, J=6.9 Hz, 1H), 2.95 (t, J=6.6 Hz, 2H), 2.38-2.26 (m, OH), 2.23-2.07 (m, 1H), 1.82-1.67 (m, 1H), 1.11 (d, J=6.8 Hz, 3H). MS-ESI (m/z) calc'd for C₂₂H₁₉N₄O₂ [M+H]⁺: 371.1. Found 371.2.

### Example 177: 2-Chloro-8-((3-(oxazol-5-yl)-1H-indazol-5-yl)oxy)-5,6,7,8-tetrahydroquinoline-3-carbonitrile, enantiomer 1 and 2

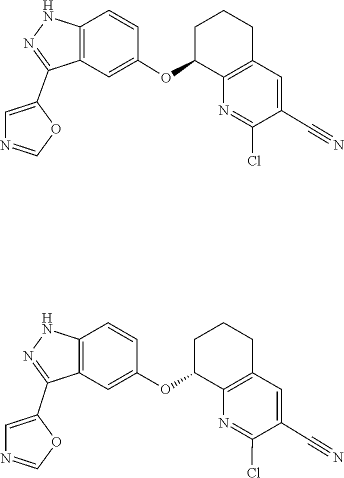 [SMILES: N#Cc1cc2c(nc1Cl)[C@@H](Oc1ccc3[nH]nc(-c4cnco4)c3c1)CCC2 ; N#Cc1cc2c(nc1Cl)[C@H](Oc1ccc3[nH]nc(-c4cnco4)c3c1)CCC2]

### Step 1: tert-Butyl 5-((2-chloro-3-cyano-5,6,7,8-tetrahydroquinolin-8-yl)oxy)-3-iodo-1H-indazole-1-carboxylate

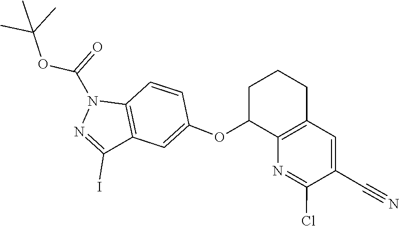 [SMILES: CC(C)(C)OC(=O)n1nc(I)c2cc(OC3CCCc4cc(C#N)c(Cl)nc43)ccc21]

Diethyl azodicarboxylate (0.19 mL, 1.2 mmol) was added to a solution of 2-chloro-8-hydroxy-5,6,7,8-tetrahydroquinoline-3-carbonitrile (250.0 mg, 1.2 mmol), tert-butyl 5-hydroxy-3-iodo-1H-indazole-1-carboxylate (431.54 mg, 1.2 mmol) and triphenylphosphine (345.71 mg, 1.32 mmol) in THF (8 mL) at 0° C. The reaction mixture was stirred for 15 minutes at 0° C., and then warmed to r.t. and stirred for 2.5 hrs. The reaction mixture was partitioned between H₂O and EtOAc, the phases were separated and the aqueous layer was extracted with EtOAc (2×). The combined organic phases were washed with H₂O (1×), dried over anhydrous Na₂SO₄ and evaporated to dryness. The residue was purified on a 55 g NH silica gel column using a 0-30% EtOAc/cyclohexane gradient eluent to afford the title compound (495 mg, 75%) as a white solid. ¹H NMR (400 MHz, DMSO-d₆) δ 8.40 (s, 1H) 7.98 (d, J=9.02 Hz, 1H) 7.39 (dd, J=9.24, 2.42 Hz, 1H) 7.26 (d, J=2.42 Hz, 1H) 5.64 (t, J=4.18 Hz, 1H) 2.89-3.00 (m, 1H) 2.74-2.86 (m, 1H) 2.18 (d, J=6.60 Hz, 1H) 2.00-2.13 (m, 1H) 1.79-1.97 (m, 2H) 1.64 (s, 9H). MS-ESI (m/z) calc'd for C₂₂H₂₁ClIN₄O₃ [M+H]⁺: 551.0. Found 551.1.

### Step 3: 2-Chloro-8-((3-(oxazol-5-yl)-1H-indazol-5-yl)oxy)-5,6,7,8-tetrahydroquinoline-3-carbonitrile, enantiomer 1 and 2

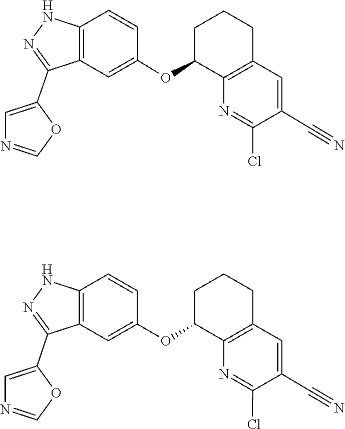 [SMILES: N#Cc1cc2c(nc1Cl)[C@@H](Oc1ccc3[nH]nc(-c4cnco4)c3c1)CCC2 ; N#Cc1cc2c(nc1Cl)[C@H](Oc1ccc3[nH]nc(-c4cnco4)c3c1)CCC2]

Prepared as described for 8-((3-(2-ethylpyridin-4-yl)-1H-indazol-5-yl)oxy)-5,6,7,8-tetrahydroquinoline-3-carbonitrile using 5-(4,4,5,5-tetramethyl-1,3,2-dioxaborolan-2-yl)-1,3-oxazole in place of 2-ethyl-4-(4,4,5,5-tetramethyl-1,3,2-dioxaborolan-2-yl)pyridine, and tert-butyl-5-[(2-chloro-3-cyano-5,6,7,8-tetrahydroquinolin-8-yl)oxy]-3-iodoindazole-1-carboxylate in place of tert-butyl 5-[(3-cyano-5,6,7,8-tetrahydroquinolin-8-yl)oxy]-3-iodo-1H-indazole-1-carboxylate, to afford 2-chloro-8-[[3-(1,3-oxazol-5-yl)-1H-indazol-5-yl]oxy]-5,6,7,8-tetrahydroquinoline-3-carbonitrile. This material was purified by chiral chromatography using Method GW to afford 2-chloro-8-[[3-(1,3-oxazol-5-yl)-1H-indazol-5-yl]oxy]-5,6,7,8-tetrahydroquinoline-3-carbonitrile, enantiomer 1 (24.4 mg, 24%) as a white solid. ¹H NMR (400 MHz, DMSO-d₆) δ 13.41 (br. s., 1H) 8.52 (s, 1H) 8.41 (s, 1H) 7.84 (s, 1H) 7.70 (d, J=1.98 Hz, 1H) 7.56 (d, J=9.02 Hz, 1H) 7.20 (dd, J=9.02, 2.42 Hz, 1H) 5.61 (t, J=3.96 Hz, 1H) 2.92-3.02 (m, 1H) 2.75-2.87 (m, 1H) 2.19-2.29 (m, 1H) 1.79-2.06 (m, 3H). MS-ESI (m/z) calc'd for C₂₀H₁₅ClN₅O₂ [M+H]⁺: 392.1. Found 392.2. A later eluting fraction was also isolated to afford 2-chloro-8-[[3-(1,3-oxazol-5-yl)-1H-indazol-5-yl]oxy]-5,6,7,8-tetrahydroquinoline-3-carbonitrile, enantiomer 2 (24.8 mg, 24%) as a white solid. ¹H NMR (400 MHz, DMSO-d₆) δ 13.41 (br. s., 1H) 8.51 (s, 1H) 8.41 (s, 1H) 7.84 (s, 1H) 7.70 (d, J=1.98 Hz, 1H) 7.56 (d, J=9.02 Hz, 1H) 7.19 (dd, J=9.02, 2.42 Hz, 1H) 5.61 (t, J=3.63 Hz, 1H) 2.91-3.03 (m, 1H) 2.74-2.88 (m, 1H) 2.18-2.28 (m, 1H) 1.77-2.06 (m, 3H). MS-ESI (m/z) calc'd for C₂₀H₁₅ClN₅O₂ [M+H]⁺: 392.1. Found 392.2.

### Example 178: 2-Chloro-8-[[3-(1,2-oxazol-4-yl)-1H-indazol-5-yl]oxy]-5,6,7,8-tetrahydroquinoline-3-carbonitrile, enantiomer 1 and 2

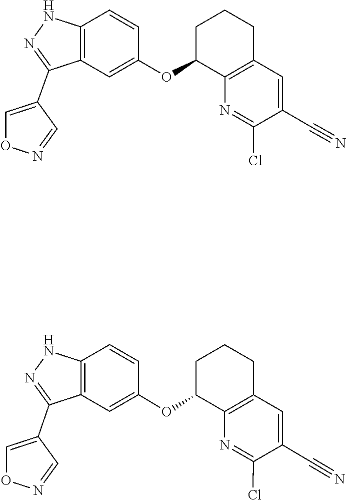 [SMILES: N#Cc1cc2c(nc1Cl)[C@@H](Oc1ccc3[nH]nc(-c4cnoc4)c3c1)CCC2 ; N#Cc1cc2c(nc1Cl)[C@H](Oc1ccc3[nH]nc(-c4cnoc4)c3c1)CCC2]

Prepared as described for 8-((3-(2-ethylpyridin-4-yl)-1H-indazol-5-yl)oxy)-5,6,7,8-tetrahydroquinoline-3-carbonitrile using isoxazole-4-boronic acid in place of 2-ethyl-4-(4,4,5,5-tetramethyl-1,3,2-dioxaborolan-2-yl)pyridine, and tert-butyl 5-[(2-chloro-3-cyano-5,6,7,8-tetrahydroquinolin-8-yl)oxy]-3-iodoindazole-1-carboxylate in place of tert-butyl 5-[(3-cyano-5,6,7,8-tetrahydroquinolin-8-yl)oxy]-3-iodo-1H-indazole-1-carboxylate, to afford 2-chloro-8-[[3-(1,2-oxazol-4-yl)-1H-indazol-5-yl]oxy]-5,6,7,8-tetrahydroquinoline-3-carbonitrile. This material was purified by chiral separation using Method GX to afford 2-chloro-8-[[3-(1,2-oxazol-4-yl)-1H-indazol-5-yl]oxy]-5,6,7,8-tetrahydroquinoline-3-carbonitrile, enantiomer 1 (12.5 mg, 12%) as a white solid. ¹H NMR (400 MHz, DMSO-d₆) δ 13.21 (br. s., 1H) 9.75 (s, 1H) 9.20 (s, 1H) 8.41 (s, 1H) 7.70 (d, J=1.98 Hz, 1H) 7.53 (d, J=9.02 Hz, 1H) 7.17 (dd, J=9.02, 2.20 Hz, 1H) 5.67 (t, J=3.74 Hz, 1H) 2.91-3.02 (m, 1H) 2.74-2.90 (m, 1H) 2.17-2.29 (m, 1H) 1.77-2.08 (m, 3H). MS-ESI (m/z) calc'd for C₂₀H₁₅ClN₅O₂ [M+H]⁺: 392.1. Found 392.1. A later eluting fraction was also isolated to afford 2-chloro-8-((3-(1,2-oxazol-4-yl)-1H-indazol-5-yl)oxy)-5,6,7,8-tetrahydroquinoline-3-carbonitrile, enantiomer 2 (10 mg, 9%) as a white solid. ¹H NMR (400 MHz, DMSO-d₆) δ 13.21 (br. s., 1H) 9.75 (s, 1H) 9.20 (s, 1H) 8.41 (s, 1H) 7.70 (d, J=1.98 Hz, 1H) 7.53 (d, J=9.02 Hz, 1H) 7.17 (dd, J=9.02, 2.20 Hz, 1H) 5.67 (t, J=3.74 Hz, 1H) 2.91-3.02 (m, 1H) 2.74-2.90 (m, 1H) 2.17-2.29 (m, 1H) 1.77-2.08 (m, 3H). MS-ESI (m/z) calc'd for C₂₀H₁₅ClN₅O₂ [M+H]⁺: 392.1. Found 392.2.

### Example 179: 2-Methoxy-8-[[3-(1,3-oxazol-5-yl)-1H-indazol-5-yl]oxy]-5,6,7,8-tetrahydroquinoline-3-carbonitrile, enantiomer 1 and 2

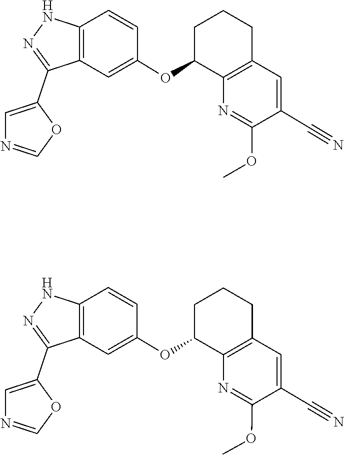 [SMILES: COc1nc2c(cc1C#N)CCC[C@@H]2Oc1ccc2[nH]nc(-c3cnco3)c2c1 ; COc1nc2c(cc1C#N)CCC[C@H]2Oc1ccc2[nH]nc(-c3cnco3)c2c1]

### Step 1: 2-Chloro-3-cyano-5,6,7,8-tetrahydroquinoline 1-oxide

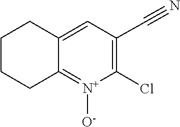 [SMILES: N#Cc1cc2c([n+]([O-])c1Cl)CCCC2]

To a solution of 2-chloro-5,6,7,8-tetrahydroquinoline-3-carbonitrile (1.0 g, 5.19 mmol) in trifluoroacetic acid (25 mL) was added a 30 wt. % solution of hydrogen peroxide in water (1.59 mL, 15.57 mmol) and the mixture was stirred at 75° C. for 15 hrs. Water was added and the solution was neutralized by addition of solid K₂CO₃ and then extracted with DCM (3×). The combined organic layers were washed with H₂O (1×), passed through a phase separator, and evaporated under reduced pressure to afford the title compound (875 mg, 81%) as a light yellow solid. ¹H NMR (400 MHz, DMSO-d₆) δ 7.78 (s, 1H) 2.79 (dt, J=16.67, 6.19 Hz, 4H) 1.77-1.92 (m, 2H) 1.63-1.74 (m, 2H). MS-ESI (m/z) calc'd for C₁₀H₁₀ClN[N⁺][O⁻] [M+H]⁺: 209.0. Found 209.0.

### Step 2: 3-Cyano-2-methoxy-5,6,7,8-tetrahydroquinoline 1-oxide

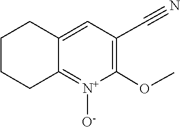

To a solution of 2-chloro-3-cyano-5,6,7,8-tetrahydroquinoline 1-oxide (875.0 mg, 4.19 mmol) in MeOH (12 mL) was added sodium methoxide (453.1 mg, 8.39 mmol) and the mixture was stirred at r.t. for three days. The reaction mixture was partitioned between water and EtOAc, the phases were separated, and the aqueous layer was extracted with EtOAc (2×). The combined organic phases were washed with brine (1×), dried over anhydrous Na₂SO₄, and evaporated to dryness. The residue was purified by silica gel column chromatography using a 0-100% EtOAc/cyclohexane gradient eluent, and then with 90% EtOAc/MeOH to afford the title compound (260 mg, 30%) as a yellow solid. ¹H NMR (400 MHz, DMSO-d₆) δ 7.60 (s, 1H) 4.15 (s, 3H) 2.77 (t, J=6.49 Hz, 2H) 2.72 (t, J=6.16 Hz, 2H) 1.76-1.86 (m, 2H) 1.63-1.72 (m, 2H). MS-ESI (m/z) calc'd for C₁₁H₁₃N[N⁺]O[O⁻][M+H]⁺: 205.1. Found 205.1.

### Step 3: 8-Hydroxy-2-methoxy-5,6,7,8-tetrahydroquinoline-3-carbonitrile

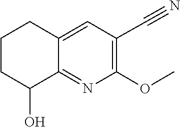

To a solution of 3-cyano-2-methoxy-5,6,7,8-tetrahydroquinoline 1-oxide (260.0 mg, 1.27 mmol) in DCM (5 mL) was added dropwise trifluoroacetic anhydride (0.53 mL, 3.82 mmol) and the mixture was stirred at 25° C. for 1 hr. The solvent was evaporated and the residue was taken up in MeOH. Then K₂CO₃ was added till basic pH and the suspension was stirred at 25° C. for 20 min. The solvent was evaporated; the residue was taken up in H₂O and extracted with DCM (3×). The combined organic layers were washed with brine (1×) passed through a phase separator and evaporated under reduced pressure to dryness. The material was purified by silica gel column chromatography using a 0-40% EtOAc/cyclohexane gradient eluent to afford the title compound (191.4 mg, 74%) as a white solid. ¹H NMR (400 MHz, DMSO-d₆) δ 8.00 (s, 1H) 5.24 (d, J=4.62 Hz, 1H) 4.48 (d, J=5.06 Hz, 1H) 3.99 (s, 3H) 2.55-2.78 (m, 2H) 1.75-1.99 (m, 3H) 1.56-1.74 (m, 1H). MS-ESI (m/z) calc'd for C₁₁H₁₃N₂O₂ [M+H]⁺: 205.1. Found 205.0.

### Step 4: tert-Butyl 5-((3-cyano-2-methoxy-5,6,7,8-tetrahydroquinolin-8-yl)oxy)-3-iodo-1H-indazole-1-carboxylate

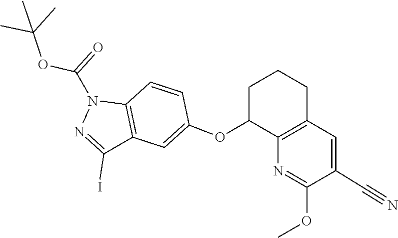 [SMILES: COc1nc2c(cc1C#N)CCCC2Oc1ccc2c(c1)c(I)nn2C(=O)OC(C)(C)C]

Diisopropyl azodicarboxylate (0.05 mL, 0.25 mmol) was added to a solution of 8-hydroxy-2-methoxy-5,6,7,8-tetrahydroquinoline-3-carbonitrile (45.0 mg, 0.21 mmol), tert-butyl 5-hydroxy-3-iodo-1H-indazole-1-carboxylate (75.39 mg, 0.21 mmol) and triphenylphosphine (109.81 mg, 0.42 mmol) in THF (3 mL) at 0° C. The reaction mixture was stirred for 15 minutes at 0° C., and then warmed to r.t. and stirred for 2.5 hrs. The reaction mixture was partitioned between H₂O and EtOAc, the phases were separated, the aqueous layer was extracted with EtOAc (2×) and the combined organic phases washed with H₂O (1×), dried over anhydrous Na₂SO₄ and evaporated to dryness. The residue was purified by silica gel column chromatography using a 0-30% EtOAc/cyclohexane gradient eluent to afford the title compound (87 mg, 76%) as a white solid. ¹H NMR (400 MHz, DMSO-d₆) δ 8.15 (s, 1H) 7.97 (d, J=9.02 Hz, 1H) 7.37-7.44 (m, 2H) 5.58 (t, J=4.40 Hz, 1H) 3.79 (s, 3H) 2.80-2.91 (m, 1H) 2.65-2.78 (m, 1H) 2.13-2.26 (m, 1H) 2.04-2.13 (m, 1H) 1.91-2.04 (m, 1H) 1.80-1.90 (m, 1H) 1.65 (s, 9H). MS-ESI (m/z) calc'd for C₂₃H₂₄IN₄O₄ [M+H]⁺: 547.1. Found 547.3.

### Step 5: 2-Methoxy-8-[[3-(1,3-oxazol-5-yl)-1H-indazol-5-yl]oxy]-5,6,7,8-tetrahydroquinoline-3-carbonitrile, enantiomer 1 and 2

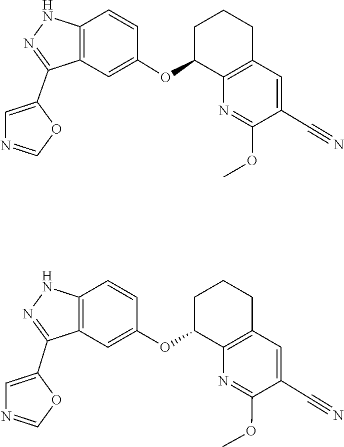 [SMILES: COc1nc2c(cc1C#N)CCC[C@@H]2Oc1ccc2[nH]nc(-c3cnco3)c2c1 ; COc1nc2c(cc1C#N)CCC[C@H]2Oc1ccc2[nH]nc(-c3cnco3)c2c1]

Prepared as described for 8-((3-(2-ethylpyridin-4-yl)-1H-indazol-5-yl)oxy)-5,6,7,8-tetrahydroquinoline-3-carbonitrile using 5-(4,4,5,5-tetramethyl-1,3,2-dioxaborolan-2-yl)-1,3-oxazole in place of 2-ethyl-4-(4,4,5,5-tetramethyl-1,3,2-dioxaborolan-2-yl)pyridine, and tert-butyl 5-[(3-cyano-2-methoxy-5,6,7,8-tetrahydroquinolin-8-yl)oxy]-3-iodoindazole-1-carboxylate in place of tert-butyl 5-[(3-cyano-5,6,7,8-tetrahydroquinolin-8-yl)oxy]-3-iodo-1H-indazole-1-carboxylate, to afford 2-methoxy-8-[[3-(1,3-oxazol-5-yl)-1H-indazol-5-yl]oxy]-5,6,7,8-tetrahydroquinoline-3-carbonitrile. This material was purified by chiral chromatography using Method GY to afford 2-methoxy-8-[[3-(1,3-oxazol-5-yl)-1H-indazol-5-yl]oxy]-5,6,7,8-tetrahydroquinoline-3-carbonitrile, enantiomer 1 (17.0 mg, 28%) as a white solid. ¹H NMR (400 MHz, DMSO-d₆) δ 13.39 (1H, br. s.) 8.51 (1H, s) 8.15 (1H, s) 7.74-7.83 (2H, m) 7.54 (1H, d, J=8.80 Hz) 7.22 (1H, dd, J=9.02, 2.20 Hz) 5.53 (1H, t, J=4.18 Hz) 3.77 (3H, s) 2.79-2.91 (1H, m) 2.68-2.78 (1H, m) 2.17-2.27 (1H, m) 1.89-2.11 (2H, m) 1.75-1.88 (1H, m). MS-ESI (m/z) calc'd for C₂₁H₁₈N₅O₃ [M+H]⁺: 388.1. Found 388.4. A later eluting fraction was also isolated to afford 2-methoxy-8-[[3-(1,3-oxazol-5-yl)-1H-indazol-5-yl]oxy]-5,6,7,8-tetrahydroquinoline-3-carbonitrile, enantiomer 2 (17.7 mg, 29%) as a white solid. ¹H NMR (400 MHz, DMSO-d₆) δ 13.39 (1H, br. s.) 8.51 (1H, s) 8.15 (1H, s) 7.73-7.83 (2H, m) 7.54 (1H, d, J=9.02 Hz) 7.22 (1H, dd, J=9.02, 2.20 Hz) 5.52 (1H, t, J=4.07 Hz) 3.77 (3H, s) 2.80-2.90 (1H, m) 2.68-2.78 (1H, m) 2.16-2.28 (1H, m) 1.90-2.10 (2H, m) 1.75-1.89 (1H, m). MS-ESI (m/z) calc'd for C₂₁H₁₈N₅O₃ [M+H]⁺: 388.1. Found 388.4.

### Example 180: 3-Fluoro-5-[[3-(1,3-oxazol-5-yl)-1H-indazol-5-yl]oxy]-5,6,7,8-tetrahydronaphthalene-2-carbonitrile, enantiomer 1 and 2

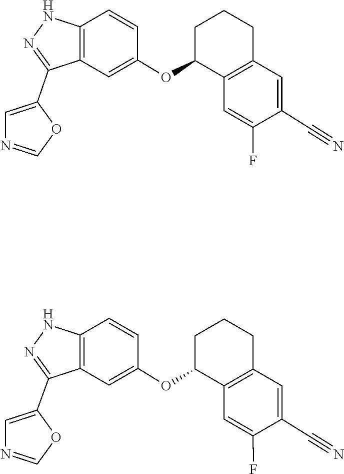 [SMILES: N#Cc1cc2c(cc1F)[C@@H](Oc1ccc3[nH]nc(-c4cnco4)c3c1)CCC2 ; N#Cc1cc2c(cc1F)[C@H](Oc1ccc3[nH]nc(-c4cnco4)c3c1)CCC2]

### Step 1: 3-Fluoro-5-oxo-5,6,7,8-tetrahydronaphthalene-2-carbonitrile

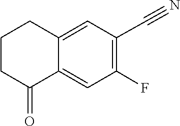 [SMILES: N#Cc1cc2c(cc1F)C(=O)CCC2]

Potassium hexacyanoferrate (II), 0.1 N standardized solution (4.94 mL, 0.49 mmol), 6-bromo-7-fluoro-3,4-dihydro-2H-naphthalen-1-one (120.0 mg, 0.49 mmol) and XPhos (18.83 mg, 0.040 mmol) were dissolved in a mixture of 1,4-dioxane (4 mL) and H₂O (1 mL) in a sealed microwave vial. The mixture was degassed with N₂ for 15 minutes. Then XPhos (18.49 mg, 0.040 mmol) and XPhos Pd G3 (32.84 mg, 0.04 mmol) were added and the mixture was stirred at 100° C. for 3 hrs. After that time an additional 0.08 eq of XPhos and 0.08 eq of XPhos Pd G3 were added and the mixture was stirred at 100° C. for 3 hrs. The reaction mixture was partitioned between H₂O and EtOAc, the phases were separated, and the aqueous layer was extracted with EtOAc (2×). The combined organic phases were washed with H₂O (1×), dried over anhydrous Na₂SO₄ and evaporated to dryness. The residue was purified by silica gel column chromatography using a 0-20% EtOAc/cyclohexane gradient eluent to afford the title compound (110 mg, 29%) as a white solid. ¹H NMR (400 MHz, DMSO-d₆) δ 8.05 (1H, d, J=6.16 Hz) 7.78 (1H, d, J=9.46 Hz) 2.97 (2H, t, J=6.05 Hz) 2.63-2.71 (2H, m) 2.01-2.15 (2H, m). MS-ESI (m/z) calc'd for C₁₁H₉FNO [M+H]⁺: 190.1. Found 190.1.

### Step 2: 3-Fluoro-5-hydroxy-5,6,7,8-tetrahydronaphthalene-2-carbonitrile

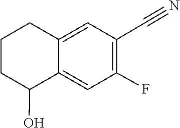 [SMILES: N#Cc1cc2c(cc1F)C(O)CCC2]

To a solution of 3-fluoro-5-oxo-5,6,7,8-tetrahydronaphthalene-2-carbonitrile (50.0 mg, 0.26 mmol) in MeOH (3 mL) was added sodium borohydride (20.0 mg, 0.53 mmol) and the mixture was stirred at 25° C. for 10 min. The solvent was evaporated and the residue was taken up in H₂O and extracted with DCM (3×). The combined organic layers were washed with water (1×), passed through a phase separator and evaporated to dryness to afford the title compound (50 mg, 99%) as a colorless oil. ¹H NMR (400 MHz, DMSO-d₆) δ 7.66 (1H, d, J=6.60 Hz) 7.45 (1H, d, J=10.56 Hz) 5.57 (1H, d, J=5.94 Hz) 4.52-4.65 (1H, m) 2.63-2.81 (2H, m) 1.93-2.03 (1H, m) 1.81-1.92 (1H, m) 1.56-1.76 (2H, m). MS-ESI (m/z) calc'd for C₁₁H₁₁FNO [M+H]⁺: 192.1. Found 192.1.

### Step 3: tert-Butyl 5-((6-cyano-7-fluoro-1,2,3,4-tetrahydronaphthalen-1-yl)oxy)-3-iodo-1H-indazole-1-carboxylate

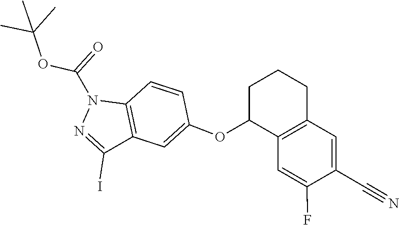 [SMILES: CC(C)(C)OC(=O)n1nc(I)c2cc(OC3CCCc4cc(C#N)c(F)cc43)ccc21]

Diisopropyl azodicarboxylate (0.06 mL, 0.31 mmol) was added to a solution of 3-fluoro-5-hydroxy-5,6,7,8-tetrahydronaphthalene-2-carbonitrile (50.0 mg, 0.26 mmol), tert-butyl 5-hydroxy-3-iodo-1H-indazole-1-carboxylate (94.18 mg, 0.26 mmol) and triphenylphosphine (137.18 mg, 0.52 mmol) in THF (4 mL) at r.t. and the reaction mixture was stirred for 2.5 hrs. After that time an additional 1 eq of PPh₃ and 0.6 eq of diisopropyl azodicarboxylate were added and the mixture was stirred at r.t. for additional 2 hrs. The reaction mixture was partitioned between H₂O and EtOAc, the phases were separated, and the aqueous layer was extracted with EtOAc (2×). The combined organic phases were washed with brine (1×), dried over anhydrous Na₂SO₄, and evaporated to dryness. The residue was purified by silica gel column chromatography using a 0-100% EtOAc/cyclohexane gradient eluent to afford the title compound as a white solid (70 mg, 50%). ¹H NMR (400 MHz, DMSO-d₆) δ 8.01 (1H, d, J=9.02 Hz) 7.81 (1H, d, J=6.60 Hz) 7.54 (1H, d, J=10.12 Hz) 7.44 (1H, dd, J=9.02, 2.42 Hz) 7.20 (1H, d, J=2.20 Hz) 5.70 (1H, t, J=5.06 Hz) 2.83-2.94 (1H, m) 2.71-2.82 (1H, m) 2.02-2.15 (1H, m) 1.76-2.01 (3H, m) 1.65 (9H, s). MS-ESI (m/z) calc'd for C₂₃H₂₂FIN₃O₃ [M+H]⁺: 534.1. Found 534.1.

### Step 4: 3-Fluoro-5-[[3-(1,3-oxazol-5-yl)-1H-indazol-5-yl]oxy]-5,6,7,8-tetrahydronaphthalene-2-carbonitrile, enantiomer 1 and 2

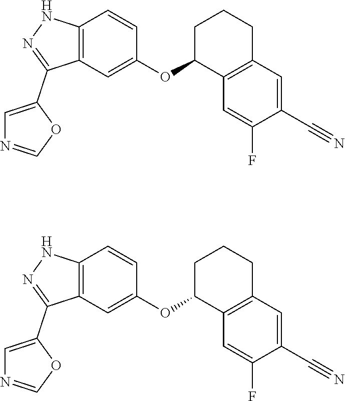 [SMILES: N#Cc1cc2c(cc1F)[C@@H](Oc1ccc3[nH]nc(-c4cnco4)c3c1)CCC2 ; N#Cc1cc2c(cc1F)[C@H](Oc1ccc3[nH]nc(-c4cnco4)c3c1)CCC2]

Prepared as described for 8-((3-(2-ethylpyridin-4-yl)-1H-indazol-5-yl)oxy)-5,6,7,8-tetrahydroquinoline-3-carbonitrile using 5-(4,4,5,5-tetramethyl-1,3,2-dioxaborolan-2-yl)-1,3-oxazole in place of 2-ethyl-4-(4,4,5,5-tetramethyl-1,3,2-dioxaborolan-2-yl)pyridine and tert-butyl 5-[(6-cyano-7-fluoro-1,2,3,4-tetrahydronaphthalen-1-yl)oxy]-3-iodoindazole-1-carboxylate in place of tert-butyl 5-[(3-cyano-5,6,7,8-tetrahydroquinolin-8-yl)oxy]-3-iodo-1H-indazole-1-carboxylate, to afford 3-fluoro-5-[[3-(1,3-oxazol-5-yl)-1H-indazol-5-yl]oxy]-5,6,7,8-tetrahydronaphthalene-2-carbonitrile. This material was purified by chiral separation using Method GZ to afford 3-fluoro-5-[[3-(1,3-oxazol-5-yl)-1H-indazol-5-yl]oxy]-5,6,7,8-tetrahydronaphthalene-2-carbonitrile, enantiomer 1 (13.1 mg, 27%) as a white solid. ¹H NMR (400 MHz, DMSO-d₆) δ 13.40 (1H, br. s.) 8.52 (1H, s) 7.87 (1H, s) 7.81 (1H, d, J=6.82 Hz) 7.66 (1H, d, J=1.98 Hz) 7.52-7.61 (2H, m) 7.23 (1H, dd, J=8.91, 2.31 Hz) 5.65 (1H, t, J=5.06 Hz) 2.83-2.98 (1H, m) 2.72-2.82 (1H, m) 1.69-2.21 (4H, m). MS-ESI (m/z) calc'd for C₂₀H₁₅FN₅O₂ [M+H]⁺: 375.1. Found 375.1. A later eluting fraction was also isolated to afford 3-fluoro-5-[[3-(1,3-oxazol-5-yl)-1H-indazol-5-yl]oxy]-5,6,7,8-tetrahydronaphthalene-2-carbonitrile, enantiomer 2 (15.1 mg, 31%) as a white solid. ¹H NMR (400 MHz, DMSO-d₆) δ 13.43 (1H, br. s.) 8.51 (1H, s) 7.86 (1H, s) 7.81 (1H, d, J=6.60 Hz) 7.66 (1H, d, J=1.98 Hz) 7.56 (2H, t, J=10.01 Hz) 7.23 (1H, dd, J=9.02, 2.20 Hz) 5.65 (1H, t, J=5.06 Hz) 2.84-2.94 (1H, m) 2.72-2.83 (1H, m) 1.75-2.11 (4H, m). MS-ESI (m/z) calc'd for C₂₀H₁₅FN₅O₂ [M+H]⁺: 375.1. Found 375.1.

### Example 181: 2-Chloro-8-[(3-cyclopropyl-1H-indazol-5-yl)oxy]-5,6,7,8-tetrahydroquinoline-3-carbonitrile, enantiomer 1 and 2

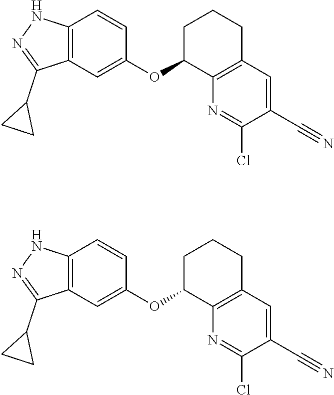 [SMILES: N#Cc1cc2c(nc1Cl)[C@@H](Oc1ccc3[nH]nc(C4CC4)c3c1)CCC2 ; N#Cc1cc2c(nc1Cl)[C@H](Oc1ccc3[nH]nc(C4CC4)c3c1)CCC2]

### Step 1: tert-Butyl 5-((2-chloro-3-cyano-5,6,7,8-tetrahydroquinolin-8-yl)oxy)-3-iodo-1H-indazole-1-carboxylate

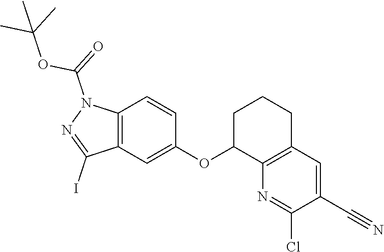 [SMILES: CC(C)(C)OC(=O)n1nc(I)c2cc(OC3CCCc4cc(C#N)c(Cl)nc43)ccc21]

Diethyl azodicarboxylate (0.13 mL, 0.810 mmol) was added to a solution of 2-chloro-8-hydroxy-5,6,7,8-tetrahydroquinoline-3-carbonitrile (170.0 mg, 0.81 mmol), tert-butyl 5-hydroxy-3-iodo-1H-indazole-1-carboxylate (293.45 mg, 0.81 mmol) and triphenylphosphine (235.09 mg, 0.90 mmol) in THF (5 mL) at 0° C. The reaction mixture was stirred for 15 minutes at 0° C., and then warmed to r.t. and stirred for 2.5 hrs. The reaction mixture was partitioned between H₂O and EtOAc, the phases were separated, and the aqueous layer was extracted with EtOAc (2×). The combined organic phases were washed with H₂O (1×), dried over anhydrous Na₂SO₄ and evaporated to dryness. The residue was purified by silica gel column chromatography using a 0-20% EtOAc/cyclohexane gradient eluent to afford material of insufficient purity which was then purified on a 28 g NH silica gel column by silica gel column chromatography using a 0-40% EtOAc/cyclohexane gradient eluent to afford the title compound (245 mg, 54%) as a white solid. ¹H NMR (400 MHz, DMSO-d₆) δ 8.35-8.42 (m, 1H) 7.98 (d, J=9.02 Hz, 1H) 7.39 (dd, J=9.02, 2.42 Hz, 1H) 7.26 (d, J=2.42 Hz, 1H) 5.64 (t, J=4.07 Hz, 1H) 2.74-3.01 (m, 2H) 1.98-2.22 (m, 2H) 1.78-1.96 (m, 2H) 1.60-1.68 (m, 9H). MS-ESI (m/z) calc'd for C₂₂H₂₁ClIN₄O₃ [M+H]⁺: 551.0. Found 551.1.

### Step 2: tert-Butyl 5-((2-chloro-3-cyano-5,6,7,8-tetrahydroquinolin-8-yl)oxy)-3-cyclopropyl-1H-indazole-1-carboxylate

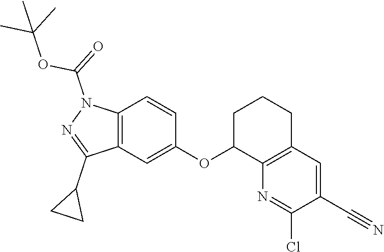 [SMILES: CC(C)(C)OC(=O)n1nc(C2CC2)c2cc(OC3CCCc4cc(C#N)c(Cl)nc43)ccc21]

tert-Butyl 5-[(2-chloro-3-cyano-5,6,7,8-tetrahydroquinolin-8-yl)oxy]-3-iodoindazole-1-carboxylate (245.0 mg, 0.44 mmol), cyclopropylboronic acid (38.21 mg, 0.44 mmol) and tripotassium phosphate (184.44 mg, 1.33 mmol) were dissolved in 1,4-dioxane (5 mL). The mixture was degassed with N₂ for 5 minutes and then Pd(dppf)Cl₂ (32.55 mg, 0.04 mmol) was added and the mixture was stirred at 100° C. under N₂ overnight. Then additional Pd(dppf)Cl₂ (32.55 mg, 0.040 mmol) and cyclopropylboronic acid (38.21 mg, 0.440 mmol) were added and the mixture was stirred at 100° C. for an additional 10 hrs. The reaction mixture was partitioned between H₂O and EtOAc, the phases were separated, and the aqueous layer was extracted with EtOAc (2×). The combined organic phases were washed with brine (1×), dried over anhydrous Na₂SO₄, and evaporated to dryness. The material was purified by column chromatography on an NH₃ column, using a 0-60% EtOAc/cyclohexane gradient eluent to afford the title compound (42 mg, 20%) as a white solid. ¹H NMR (400 MHz, DMSO-d₆) δ 8.40 (s, 1H) 7.91 (d, J=9.02 Hz, 1H) 7.65 (d, J=2.42 Hz, 1H) 7.29 (dd, J=9.02, 2.42 Hz, 1H) 5.58 (t, J=4.07 Hz, 1H) 2.89-3.00 (m, 2H) 2.74-2.85 (m, 2H) 2.31-2.39 (m, 1H) 2.19 (d, J=6.60 Hz, 1H) 2.02 (dd, J=14.31, 10.78 Hz, 2H) 1.78-1.94 (m, 3H) 1.62 (s, 9H). MS-ESI (m/z) calc'd for C₂₅H₂₆ClN₄O₃ [M+H]⁺: 465.2. Found 465.2.

### Step 3: 2-Chloro-8-[(3-cyclopropyl-1H-indazol-5-yl)oxy]-5,6,7,8-tetrahydroquinoline-3-carbonitrile, enantiomer 1 and 2

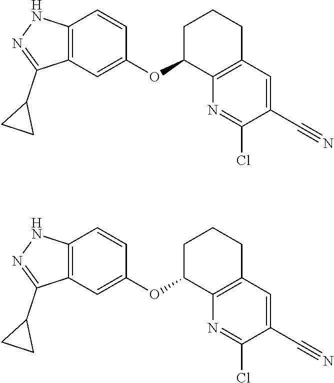 [SMILES: N#Cc1cc2c(nc1Cl)[C@@H](Oc1ccc3[nH]nc(C4CC4)c3c1)CCC2 ; N#Cc1cc2c(nc1Cl)[C@H](Oc1ccc3[nH]nc(C4CC4)c3c1)CCC2]

Prepared as described for 5-[(3-cyclopropyl-1H-indazol-5-yl)oxy]-5,6,7,8-tetrahydroquinoline-2-carbonitrile, using tert-butyl 5-[(2-chloro-3-cyano-5,6,7,8-tetrahydroquinolin-8-yl)oxy]-3-cyclopropylindazole-1-carboxylate (40 mg, 0.09 mmol) in place of tert-butyl 5-[(2-cyano-5,6,7,8-tetrahydroquinolin-5-yl)oxy]-3-cyclopropyl-1H-indazole-1-carboxylate, to afford 2-chloro-8-[(3-cyclopropyl-1H-indazol-5-yl)oxy]-5,6,7,8-tetrahydroquinoline-3-carbonitrile. This material was purified by chiral separation using Method HA to afford 2-chloro-8-[(3-cyclopropyl-1H-indazol-5-yl)oxy]-5,6,7,8-tetrahydroquinoline-3-carbonitrile, enantiomer 1 (9 mg, 28%) as a white solid. ¹H NMR (400 MHz, DMSO-d₆) δ 12.41 (s, 1H) 8.38 (s, 1H) 7.46 (d, J=1.98 Hz, 1H) 7.36 (d, J=9.02 Hz, 1H) 7.05 (dd, J=8.91, 2.31 Hz, 1H) 5.31-5.49 (m, 1H) 2.72-3.01 (m, 2H) 2.16-2.29 (m, 2H) 1.74-2.04 (m, 2H) 1.16-1.27 (m, 1H) 0.91-1.00 (m, 4H). MS-ESI (m/z) calc'd for C₂₀H₁₈ClNO₄ [M+H]⁺: 365.1. Found 365.2. A later eluting fraction was also isolated to afford 2-chloro-8-[(3-cyclopropyl-1H-indazol-5-yl)oxy]-5,6,7,8-tetrahydroquinoline-3-carbonitrile, enantiomer 2 (9 mg, 29%) as a white solid. ¹H NMR (400 MHz, DMSO-d₆) δ 12.41 (s, 1H) 8.38 (s, 1H) 7.46 (d, J=1.98 Hz, 1H) 7.36 (d, J=8.80 Hz, 1H) 7.05 (dd, J=9.02, 2.20 Hz, 1H) 5.37-5.49 (m, 1H) 2.73-3.01 (m, 2H) 2.15-2.29 (m, 2H) 1.83-1.98 (m, 2H) 1.19-1.27 (m, 1H) 0.89-1.00 (m, 4H). MS-ESI (m/z) calc'd for C₂₀H₁₈ClNO₄ [M+H]⁺: 365.1. Found 365.2.

### Example 182: 5-((3-(Oxazol-5-yl)-1H-indazol-5-yl)oxy)-6,7-dihydro-5H-cyclopenta[b]pyrazine-2-carbonitrile, enantiomer 1 and 2

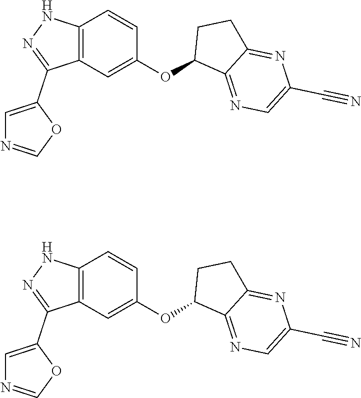 [SMILES: N#Cc1cnc2c(n1)CC[C@@H]2Oc1ccc2[nH]nc(-c3cnco3)c2c1 ; N#Cc1cnc2c(n1)CC[C@H]2Oc1ccc2[nH]nc(-c3cnco3)c2c1]

### Step 1: 1,5,6,7-Tetrahydro-2H-cyclopenta[b]pyrazin-2-one

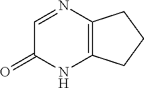

10 M sodium hydroxide (1.27 mL, 12.74 mmol) was added to a suspension of glycinamide hydrochloride (563.4 mg, 5.1 mmol) and cyclopentane-1,2-dione (500.0 mg, 5.1 mmol) in EtOH (25 mL) at 25° C. The mixture was then gradually warmed to 80° C. and stirred for 3 hrs. Then the reaction mixture was filtered and the filtrate was concentrated under reduced pressure to give a residue. The residue was purified by silica gel column chromatography using a 0-10% MeOH/DCM gradient eluent to afford the title compound (146 mg, 21%) as a yellow solid. ¹H NMR (400 MHz, DMSO-d₆) δ 7.74 (s, 1H), 2.85-2.69 (m, 4H), 2.08 (p, J=7.7 Hz, 2H). MS-ESI (m/z) calc'd for C₇H₉N₂O [M+H]⁺: 137.1. Found 137.0.

### Step 2: 2-Chloro-6,7-dihydro-5H-cyclopenta[b]pyrazine

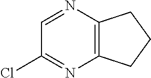

A suspension of 1,5,6,7-tetrahydro-2H-cyclopenta[b]pyrazin-2-one (145.0 mg, 1.01 mmol) and tetraethylammonium chloride (167.65 mg, 1.01 mmol) in phosphorus oxychloride (1.46 mL, 15.65 mmol) was heated at 100° C. for 1 hr using microwave irradiation. The solvent was evaporated to give a residue that was taken up in saturated aqueous NaHCO₃ and extracted with DCM (3×). The combined organic layers were passed through a phase separator and evaporated to give a residue which was purified by silica gel column chromatography using a 0-25% EtOAc/cyclohexane gradient eluent to afford the title compound (70 mg, 45%) as a colorless oil. ¹H NMR (400 MHz, DMSO-d₆) δ 8.44 (t, J=1.1 Hz, 1H), 3.04-2.89 (m, 4H), 2.27-2.08 (m, 2H). MS-ESI (m/z) calc'd for C₇H₈ClN₂ [M+H]⁺: 155.0. Found 155.0.

### Step 3: 3-Chloro-6,7-dihydro-5H-cyclopenta[b]pyrazine 1-oxide

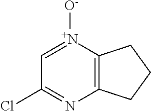 [SMILES: [O-][n+]1cc(Cl)nc2c1CCC2]

To a solution of 3-chloro-6,7-dihydro-5H-cyclopenta[b]pyrazine (1.05 g, 6.79 mmol) in DCE (33.96 mL) was added MCPBA (1.41 g, 8.15 mmol) and the mixture was stirred at 65° C. for 1.5 hrs. After cooling the mixture was diluted with DCM and washed with saturated aqueous NaHCO₃. The organic phase was passed through a phase separator and evaporated to afford the title compound (1.15 g, 99%) as a yellow solid. ¹H NMR (500 MHz, DMSO-d₆) δ 1.83-2.00 (m, 1H), 2.42-2.53 (m, 1H), 2.78-2.91 (m, 1H), 2.95-3.08 (m, 1H), 5.01 (dt, J=7.5, 5.8 Hz, 1H), 5.69 (d, J=6.0 Hz, 1H), 8.57 (s, 1H). MS-ESI (m/z) calc'd for C₇H₈ClN[N⁺][O⁻] [M+H]⁺: 171.0. Found 171.0.

### Step 4: 2-Chloro-6,7-dihydro-5H-cyclopenta[b]pyrazin-5-ol

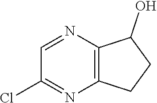 [SMILES: OC1CCc2nc(Cl)cnc21]

To a solution of 3-chloro-6,7-dihydro-5H-cyclopenta[b]pyrazine 1-oxide (1.15 g, 6.74 mmol) in DCM (33.73 mL) was added trifluoroacetic anhydride (2.81 mL, 20.22 mmol) dropwise and the mixture was stirred at 40° C. for 3 days. The solvent was evaporated to give a residue that was taken up in MeOH. K₂CO₃ (932 mg, 6.74 mmol) was added and the suspension was stirred at 25° C. for 1 hr. The solvent was evaporated and the residue was taken up in water and extracted with DCM (3×). The combined organic layers were passed through a phase separator and evaporated to afford a residue which was purified by silica gel column chromatography using a 0-50% EtOAc/cyclohexane gradient to obtain a yellow solid which was further purified by silica gel column chromatography using a 0-100% EtOAc/cyclohexane gradient eluent to afford the title compound (435 mg, 38%) as a clear oil. ¹H NMR (500 MHz, DMSO-d₆) δ 1.83-2.00 (m, 1H), 2.42-2.53 (m, 1H), 2.78-2.91 (m, 1H), 2.95-3.08 (m, 1H), 5.01 (dt, J=7.5, 5.8 Hz, 1H), 5.69 (d, J=6.0 Hz, 1H), 8.57 (s, 1H). MS-ESI (m/z) calc'd for C₇H₈ClN₂O [M+H]⁺: 171.0. Found 171.0.

### Step 5: 5-(5-((2-Chloro-6,7-dihydro-5H-cyclopenta[b]pyrazin-5-yl)oxy)-1-((2-(trimethylsilyl)ethoxy)methyl)-1H-indazol-3-yl)oxazole

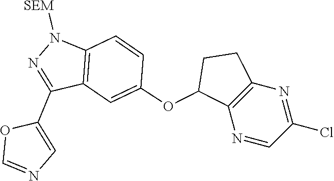 [SMILES: C[Si](C)(C)CCOCn1nc(-c2cnco2)c2cc(OC3CCc4nc(Cl)cnc43)ccc21]

Diisopropyl azodicarboxylate (0.14 mL, 0.70 mmol) was added to a solution of 2-chloro-6,7-dihydro-5H-cyclopenta[b]pyrazin-5-ol (100.0 mg, 0.59 mmol), 3-(oxazol-5-yl)-1-((2-(trimethylsilyl)ethoxy)methyl)-1H-indazol-5-ol (194.28 mg, 0.59 mmol) and triphenylphosphine (307.49 mg, 1.17 mmol) in THF (5.862 mL) dropwise and the mixture was stirred at r.t. for 18 hrs. The reaction mixture was diluted with H₂O (50 mL) and EtOAc (3×50 mL). The organic phase was separated, dried over Na₂SO₄, filtered, and concentrated under reduced pressure to give a residue. The residue was purified by silica gel column chromatography using a 0-50% EtOAc/cyclohexane gradient eluent to afford the title compound (283 mg, 100%), as a white solid. ¹H NMR (400 MHz, DMSO-d₆) δ 8.66 (s, 1H), 8.56 (s, 1H), 7.90 (s, 1H), 7.78 (d, J=9.1 Hz, 1H), 7.72 (d, J=2.3 Hz, 1H), 7.28 (dd, J=9.0, 2.3 Hz, 1H), 6.06 (dd, J=7.3, 4.2 Hz, 1H), 5.80 (s, 2H), 3.59-3.54 (m, 2H), 3.25-3.13 (m, 1H), 3.11-2.98 (m, 1H), 2.89-2.74 (m, 1H), 2.27 (ddt, J=13.8, 9.4, 5.0 Hz, 1H), 0.83 (t, J=8.0 Hz, 2H), −0.09 (s, 9H). MS-ESI (m/z) calc'd for C₂₃H₂₇ClN₅O₃Si [M+H]⁺: 484.2. Found 484.1.

### Step 6: 5-((3-(Oxazol-5-yl)-1-((2-(trimethylsilyl)ethoxy)methyl)-1H-indazol-5-yl)oxy)-6,7-dihydro-5H-cyclopenta[b]pyrazine-2-carbonitrile

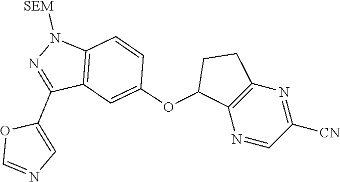 [SMILES: C[Si](C)(C)CCOCn1nc(-c2cnco2)c2cc(OC3CCc4nc(C#N)cnc43)ccc21]

Potassium hexacyanoferrate (II), 0.1 N (2.92 mL, 0.29 mmol), 5-(5-((2-chloro-6,7-dihydro-5H-cyclopenta[b]pyrazin-5-yl)oxy)-1-((2-(trimethylsilyl)ethoxy)methyl)-1H-indazol-3-yl)oxazole (283.0 mg, 0.58 mmol) and KOAc (57.38 mg, 0.58 mmol) were dissolved in a mixture of 1,4-dioxane (3.1 mL) and H₂O (1 mL) in a sealed microwave vial. The mixture was degassed with N₂ for 15 minutes. Then XPhos (18.49 mg, 0.04 mmol) and XPhos Pd G3 (49.49 mg, 0.060 mmol) were added and the mixture was stirred at 110° C. for 2 hrs. The reaction was brought to r.t. and then diluted with saturated aqueous NaHCO₃ (100 mL) and EtOAc (100 mL). The organic phase was separated, dried over Na₂SO₄, filtered, and concentrated under reduced pressure to give a residue. The residue was purified by silica gel column chromatography using a 0-100% EtOAc/cyclohexane gradient eluent to afford the title compound (41 mg, 15%) as a yellow solid. ¹H NMR (400 MHz, MeOD) δ 8.90 (s, 1H), 8.40 (s, 1H), 7.84 (d, J=2.3 Hz, 1H), 7.74 (s, 1H), 7.69 (d, J=9.1 Hz, 1H), 7.33 (dd, J=9.1, 2.3 Hz, 1H), 6.02 (dd, J=7.4, 4.7 Hz, 1H), 5.81 (s, 2H), 3.66-3.59 (m, 2H), 3.39-3.35 (m, 1H), 3.24-3.09 (m, 1H), 2.95-2.80 (m, 1H), 2.46 (ddt, J=14.1, 9.1, 5.3 Hz, 1H), 0.92-0.87 (m, 2H), −0.06 (s, 9H). MS-ESI (m/z) calc'd for C₂₄H₂₇N₆O₃Si [M+H]⁺: 475.2. Found 475.2.

### Step 7: 5-((3-(Oxazol-5-yl)-1H-indazol-5-yl)oxy)-6,7-dihydro-5H-cyclopenta[b]pyrazine-2-carbonitrile, enantiomer 1 and 2

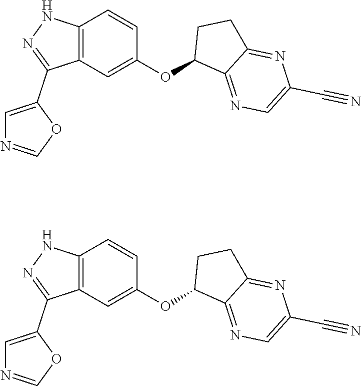 [SMILES: N#Cc1cnc2c(n1)CC[C@@H]2Oc1ccc2[nH]nc(-c3cnco3)c2c1 ; N#Cc1cnc2c(n1)CC[C@H]2Oc1ccc2[nH]nc(-c3cnco3)c2c1]

Prepared as described for 1-methoxy-5-((3-(oxazol-5-yl)-1H-indazol-5-yl)amino)-5,6,7,8-tetrahydronaphthalene-2-carbonitrile using 5-((3-(oxazol-5-yl)-1-((2-(trimethylsilyl)ethoxy)methyl)-1H-indazol-5-yl)oxy)-6,7-dihydro-5H-cyclopenta[b]pyrazine-2-carbonitrile in place of 1-methoxy-5-{[3-(1,3-oxazol-5-yl)-1-{[2-(trimethylsilyl)ethoxy]methyl}-1H-indazol-5-yl]amino}-5,6,7,8-tetrahydronaphthalene-2-carbonitrile to afford 7-[[3-(1,3-oxazol-5-yl)-1H-indazol-5-yl]oxy]-6,7-dihydro-5H-cyclopenta[b]pyrazine-3-carbonitrile, which was subjected to chiral separation using Method HC to afford 7-[[3-(1,3-oxazol-5-yl)-1H-indazol-5-yl]oxy]-6,7-dihydro-5H-cyclopenta[b]pyrazine-3-carbonitrile, enantiomer 1 (2.3 mg, 8%) as a white solid. ¹H NMR (400 MHz, MeOD) δ 8.87 (s, 1H), 8.36 (s, 1H), 7.78 (d, J=2.3 Hz, 1H), 7.68 (s, 1H), 7.53 (d, J=9.1 Hz, 1H), 7.24 (dd, J=9.1, 2.3 Hz, 1H), 5.97 (dd, J=7.3, 4.7 Hz, 1H), 3.36-3.24 (m, 1H), 3.14 (dddd, J=17.9, 8.9, 5.9, 1.0 Hz, 1H), 2.85 (dddd, J=14.3, 8.9, 7.3, 5.6 Hz, 1H), 2.43 (dddd, J=13.8, 9.0, 5.9, 4.7 Hz, 1H). MS-ESI (m/z) calc'd for C₁₈H₁₃N₆O₂ [M+H]⁺: 345.1 Found 345.1. A later eluting fraction was also isolated to afford 7-[[3-(1,3-oxazol-5-yl)-1H-indazol-5-yl]oxy]-6,7-dihydro-5H-cyclopenta[b]pyrazine-3-carbonitrile, enantiomer 2 (2.8 mg, 10%) as a white solid. ¹H NMR (400 MHz, MeOD) δ 8.87 (s, 1H), 8.36 (s, 1H), 7.78 (d, J=2.3 Hz, 1H), 7.68 (s, 1H), 7.53 (d, J=9.1 Hz, 1H), 7.24 (dd, J=9.0, 2.3 Hz, 1H), 5.97 (dd, J=7.3, 4.7 Hz, 1H), 3.30-3.26 (m, 1H), 3.14 (dddd, J=17.9, 8.9, 6.0, 1.0 Hz, 1H), 2.85 (dddd, J=14.3, 8.9, 7.3, 5.6 Hz, 1H), 2.43 (dddd, J=13.9, 9.1, 5.9, 4.7 Hz, 1H). MS-ESI (m/z) calc'd for C₁₈H₁₃N₆O₂ [M+H]⁺: 345.1 Found 345.2.

### Example 183: 2-Chloro-8-[(3-cyclopropyl-1H-indazol-5-yl)amino]-4-methyl-5,6,7,8-tetrahydroquinoline-3-carbonitrile, enantiomer 1 and 2

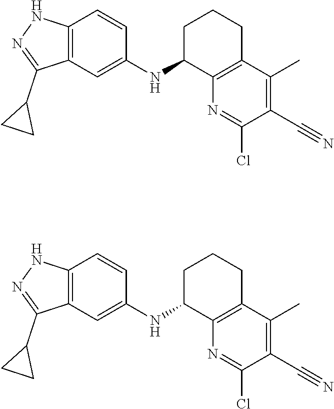 [SMILES: Cc1c(C#N)c(Cl)nc2c1CCC[C@@H]2Nc1ccc2[nH]nc(C3CC3)c2c1 ; Cc1c(C#N)c(Cl)nc2c1CCC[C@H]2Nc1ccc2[nH]nc(C3CC3)c2c1]

### Step 1: 2-Chloro-4-methyl-8-oxo-5,6,7,8-tetrahydroquinoline-3-carbonitrile

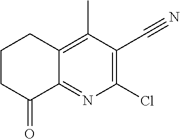

A solution of 2-chloro-8-hydroxy-4-methyl-5,6,7,8-tetrahydroquinoline-3-carbonitrile (230.0 mg, 1.03 mmol) in DCM (5 mL) was treated with Dess-Martin periodinane (525.72 mg, 1.24 mmol) and stirred at r.t. for 2 hrs. The reaction was diluted with DCM and quenched by addition of 2 mL of saturated aqueous NaHCO₃. After stirring at r.t. for 5 minutes, the phases were separated. The aqueous layer was extracted with DCM (2×) and the combined organic phases were washed with H₂O (1×), passed through a phase separator and evaporated to dryness. The residue was purified by silica gel column chromatography using a 0-80% EtOAc/cyclohexane gradient eluent to afford the title compound (220 mg, 96%) as a white solid. ¹H NMR (400 MHz, DMSO-d₆) δ 2.95 (t, J=6.05 Hz, 2H) 2.69-2.78 (m, 2H) 2.57 (s, 3H) 2.12 (quin, J=6.44 Hz, 2H). MS-ESI (m/z) calc'd for C₁₁H₁₀ClN₂O [M+H]⁺: 221.0. Found 221.1.

### Step 2: 2-Chloro-8-((3-iodo-1H-indazol-5-yl)amino)-4-methyl-5,6,7,8-tetrahydroquinoline-3-carbonitrile

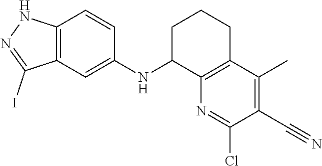 [SMILES: Cc1c(C#N)c(Cl)nc2c1CCCC2Nc1ccc2[nH]nc(I)c2c1]

To a stirred solution of 3-iodo-1H-indazol-5-amine (309.94 mg, 1.2 mmol) and 2-chloro-4-methyl-8-oxo-6,7-dihydro-5H-quinoline-3-carbonitrile (220.0 mg, 1 mmol) in 1,4-dioxane (8 mL) was added 4-methylbenzenesulfonic acid hydrate (18.97 mg, 0.10 mmol) and the mixture was stirred at 100° C. for 4 hrs. The reaction was cooled to 40° C., sodium triacetoxyborohydride (575.08 mg, 2.99 mmol) was added portionwise over 3 hrs and the mixture was stirred overnight at 40° C. The mixture was cooled to r.t. and sodium borohydride (113.16 mg, 2.99 mmol) was added and the reaction mixture was stirred at r.t. for 2 hrs. The reaction mixture was partitioned between H₂O and EtOAc, the phases were separated, and the aqueous layer was extracted with EtOAc (2×). The combined organic phases were washed with H₂O (1×), dried over anhydrous Na₂SO₄, and evaporated to dryness. The residue was purified by silica gel column chromatography using a 0-50% EtOAc/cyclohexane gradient eluent to afford the title compound (188 mg, 41%) as a yellow solid. ¹H NMR (400 MHz, DMSO-d₆) δ 13.08 (s, 1H) 7.31 (d, J=8.80 Hz, 1H) 6.99 (dd, J=9.02, 1.98 Hz, 1H) 6.52 (s, 1H) 5.96 (d, J=7.48 Hz, 1H) 4.63 (br. s., 1H) 2.73-2.87 (m, 1H) 2.59-2.71 (m, 1H) 2.50 (3H, 1.77-1.98 (m, 4H). MS-ESI (m/z) calc'd for C₁₈H₁₆ClIN₅ [M+H]⁺: 464.0. Found 464.1.

### Step 3: tert-Butyl 5-((2-chloro-3-cyano-4-methyl-5,6,7,8-tetrahydroquinolin-8-yl)amino)-3-iodo-1H-indazole-1-carboxylate

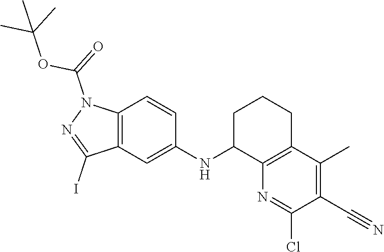 [SMILES: Cc1c(C#N)c(Cl)nc2c1CCCC2Nc1ccc2c(c1)c(I)nn2C(=O)OC(C)(C)C]

To a solution of 2-chloro-8-[(3-iodo-1H-indazol-5-yl)amino]-4-methyl-5,6,7,8-tetrahydroquinoline-3-carbonitrile (188.0 mg, 0.41 mmol) and triethylamine (0.08 mL, 0.61 mmol) in THF (5 mL), was added di-tert-butyl dicarbonate (309.7 mg, 1.42 mmol) and the mixture was stirred at r.t. overnight. The reaction mixture was partitioned between water and EtOAc, the phases were separated, the aqueous layer was extracted with EtOAc (2×) and the combined organic phases were washed with brine (1×), dried over anhydrous Na₂SO₄, and evaporated to dryness. The material was purified by silica gel column chromatography on a 28 g NH column using a 0-30% EtOAc/cyclohexane gradient eluent to afford the title compound (80 mg, 35%) as a yellow solid. ¹H NMR (400 MHz, DMSO-d₆) δ 7.80 (d, J=9.02 Hz, 1H) 7.15 (dd, J=9.02, 2.20 Hz, 1H) 6.66 (d, J=2.20 Hz, 1H) 6.36 (d, J=7.70 Hz, 1H) 4.72 (br. s., 1H) 2.75-2.86 (m, 1H) 2.68 (s, 1H) 2.50 (3H, peak under DMSO signal) 1.82-2.05 (m, 4H) 1.64 (s, 9H). MS-ESI (m/z) calc'd for C₂₃H₂₄ClIN₅O₂ [M+H]⁺: 564.1. Found 564.2.

### Step 4: tert-Butyl 5-((2-chloro-3-cyano-4-methyl-5,6,7,8-tetrahydroquinolin-8-yl)amino)-3-cyclopropyl-1H-indazole-1-carboxylate

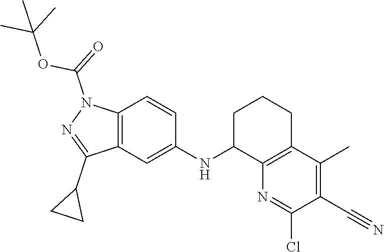 [SMILES: Cc1c(C#N)c(Cl)nc2c1CCCC2Nc1ccc2c(c1)c(C1CC1)nn2C(=O)OC(C)(C)C]

tert-Butyl 5-[(2-chloro-3-cyano-4-methyl-5,6,7,8-tetrahydroquinolin-8-yl)amino]-3-iodoindazole-1-carboxylate (80.0 mg, 0.14 mmol), cyclopropylboronic acid (14.63 mg, 0.17 mmol) and tripotassium phosphate (58.83 mg, 0.43 mmol) were dissolved in 1,4-dioxane (2 mL) and the mixture was degassed with N₂ for 5 minutes. Pd(dppf)Cl₂ (9.28 mg, 0.01 mmol) was then added and the mixture was stirred at 100° C. under N₂ for 2 hrs. The reaction mixture was partitioned between H₂O and EtOAc, the phases were separated, the aqueous layer was extracted with EtOAc (2×) and the combined organic phases were washed with brine (1×), dried over anhydrous Na₂SO₄, and evaporated to dryness. The residue was purified by silica gel column chromatography on a 11 g NH column using a 0-25% EtOAc/cyclohexane gradient eluent to afford the title compound (35 mg, 52%) as a yellow solid. ¹H NMR (400 MHz, DMSO-d₆) δ 7.65-7.79 (m, 1H) 6.96-7.11 (m, 2H) 6.13 (d, J=7.70 Hz, 1H) 4.72 (br. s., 1H) 2.74-2.87 (m, 1H) 2.62-2.72 (m, 1H) 2.50 (3H, peak under DMSO signal) 2.20-2.30 (m, 1H) 1.80-2.03 (m, 4H) 1.62 (s, 9H) 0.95-1.10 (m, 4H). MS-ESI (m/z) calc'd for C₂₆H₂₈ClN₅O₂ [M+H]⁺: 478.2. Found 478.3.

### Step 5: 2-Chloro-8-[(3-cyclopropyl-1H-indazol-5-yl)amino]-4-methyl-5,6,7,8-tetrahydroquinoline-3-carbonitrile, enantiomer 1 and 2

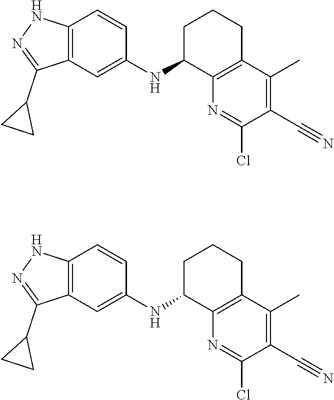 [SMILES: Cc1c(C#N)c(Cl)nc2c1CCC[C@@H]2Nc1ccc2[nH]nc(C3CC3)c2c1 ; Cc1c(C#N)c(Cl)nc2c1CCC[C@H]2Nc1ccc2[nH]nc(C3CC3)c2c1]

Prepared as described for 5-[(3-cyclopropyl-1H-indazol-5-yl)oxy]-5,6,7,8-tetrahydroquinoline-2-carbonitrile, using tert-butyl 5-[(2-chloro-3-cyano-4-methyl-5,6,7,8-tetrahydroquinolin-8-yl)amino]-3-cyclopropylindazole-1-carboxylate) in place of tert-butyl 5-[(2-cyano-5,6,7,8-tetrahydroquinolin-5-yl)oxy]-3-cyclopropyl-1H-indazole-1-carboxylate, to afford 2-chloro-8-[(3-cyclopropyl-1H-indazol-5-yl)amino]-4-methyl-5,6,7,8-tetrahydroquinoline-3-carbonitrile, which was subjected to chiral separation using Method HD to afford 2-chloro-8-[(3-cyclopropyl-1H-indazol-5-yl)amino]-4-methyl-5,6,7,8-tetrahydroquinoline-3-carbonitrile, enantiomer 1 (1.1 mg, 4%) as a white solid. ¹H NMR (400 MHz, DMSO-d₆) δ 12.09 (s, 1H) 7.20 (d, J=8.80 Hz, 1H) 6.78-6.98 (m, 2H) 5.65 (d, J=7.26 Hz, 1H) 4.56-4.66 (m, 1H) 2.74-2.86 (m, 1H) 2.60-2.72 (m, 1H) 2.50 (3H, peak under DMSO signal) 2.10-2.22 (m, 1H) 1.79-2.06 (m, 4H) 0.84-0.99 (m, 4H). MS-ESI (m/z) calc'd for C₂₁H₂₁ClN₅ [M+H]⁺: 378.1. Found 378.2. A later eluting fraction was also isolated to afford 2-chloro-8-[(3-cyclopropyl-1H-indazol-5-yl)amino]-4-methyl-5,6,7,8-tetrahydroquinoline-3-carbonitrile, enantiomer 2 (0.6 mg, 2%) as a white solid. ¹H NMR (400 MHz, DMSO-d₆) δ 12.09 (br. s., 1H) 7.20 (d, J=8.80 Hz, 1H) 6.82-7.02 (m, 2H) 5.65 (d, J=7.26 Hz, 1H) 4.59-4.66 (m, 1H) 2.72-2.87 (m, 1H) 2.60-2.71 (m, 1H) 2.50 (3H, peak under DMSO signal) 2.10-2.21 (m, 1H) 1.71-2.06 (m, 4H) 0.82-1.01 (m, 4H). MS-ESI (m/z) calc'd for C₂₁H₂₁ClN₅ [M+H]⁺: 378.1. Found 378.2.

### Example 184: 2-Chloro-8-[(3-cyclopropyl-1H-indazol-5-yl)oxy]-4-methyl-5,6,7,8-tetrahydroquinoline-3-carbonitrile, enantiomer 1 and 2

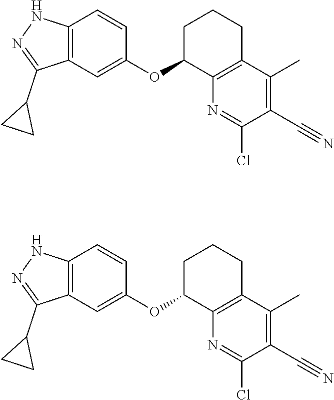 [SMILES: Cc1c(C#N)c(Cl)nc2c1CCC[C@@H]2Oc1ccc2[nH]nc(C3CC3)c2c1 ; Cc1c(C#N)c(Cl)nc2c1CCC[C@H]2Oc1ccc2[nH]nc(C3CC3)c2c1]

### Step 1: 4-Methyl-2-oxo-2,4a,5,6,7,8-hexahydroquinoline-3-carbonitrile

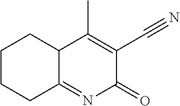

To a solution of 2-cyanoacetic acid ethyl ester (8.51 mL, 80 mmol), acetaldehyde (4.49 mL, 80 mmol), and cyclohexanone (8.28 mL, 80 mmol) in DMSO (12 mL) was added pyrrolidine (0.67 mL, 8 mmol) and the mixture was stirred for 1 hr. Ammonium acetate (9.25 g, 120 mmol) was then added and the mixture was stirred vigorously for 30 min. Pyrrolidine (8.03 mL, 96 mmol) was then added and the reaction mixture was stirred at 80° C. overnight. The reaction mixture was partitioned between water and DCM, the phases were separated, and the aqueous layer was extracted with DCM (2×). The combined organic phases were washed with H₂O (1×), passed through a phase separator and evaporated to dryness. The residue was purified by silica gel column chromatography on a 240 g C18 column using a 5-35% MeCN/H₂O (0.1% HCOOH) to afford the title compound (5.12 g, 34%) as a beige solid. ¹H NMR (400 MHz, DMSO-d₆) δ 12.07 (br. s., 1H) 2.55 (br. s., 2H) 2.34-2.41 (m, 2H) 2.28 (s, 3H) 1.66-1.72 (m, 4H). MS-ESI (m/z) calc'd for C₁₁H₁₃N₂₀ [M+H]⁺: 189.1. Found 189.0.

### Step 2: 2-Chloro-4-methyl-5,6,7,8-tetrahydroquinoline-3-carbonitrile

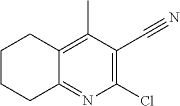

A suspension of 4-methyl-2-oxo-2,4a,5,6,7,8-hexahydroquinoline-3-carbonitrile (5.12 g, 27.2 mmol) in phosphorus(V) oxychloride (25.0 mL, 267.4 mmol) was heated at 100° C. for 4 hrs.

The excess of POCl₃ was evaporated and the brown oily residue was partitioned between H₂O and DCM. The phases were separated, and the aqueous layer was extracted with DCM (2×). The combined organic phases were washed with H₂O (1×), dried over anhydrous Na₂SO₄, and evaporated to dryness. The residue was purified by silica gel column chromatography using a 0-30% EtOAc/cyclohexane gradient eluent to afford the title compound (1.22 g, 22%) as a white solid. ¹H NMR (400 MHz, DMSO-d₆) δ 2.78-2.89 (m, 2H) 2.63-2.74 (m, 2H) 2.41 (s, 3H) 1.67-1.84 (m, 4H). MS-ESI (m/z) calc'd for C₁₁H₁₂ClN₂ [M+H]⁺: 207.1. Found 207.04.

### Step 3: 2-Chloro-3-cyano-4-methyl-5,6,7,8-tetrahydroquinoline 1-oxide

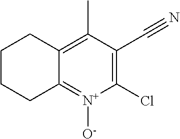

To a solution of 2-chloro-4-methyl-5,6,7,8-tetrahydroquinoline-3-carbonitrile (310.0 mg, 1.5 mmol) in trifluoroacetic acid (5 mL) was added a 30 wt. % solution of hydrogen peroxide in water (0.15 mL, 4.5 mmol) and the mixture was stirred at 75° C. for 16 hrs. Water was added and the solution was neutralized by addition of solid K₂CO₃, and then extracted with DCM (3×). The combined organic layers were washed with H₂O (1×), passed through a phase separator, and evaporated under reduced pressure to afford the title compound (190 mg, 57%) as a light yellow solid. ¹H NMR (400 MHz, DMSO-d₆) δ 2.78-2.91 (m, 2H) 2.62-2.75 (m, 2H) 2.41 (s, 3H) 1.66-1.84 (m, 4H). MS-ESI (m/z) calc'd for C₁₁H₁₂ClN[N⁺][O⁻] [M+H]⁺: 223.1. Found 223.1.

### Step 4: 2-Chloro-8-hydroxy-4-methyl-5,6,7,8-tetrahydroquinoline-3-carbonitrile

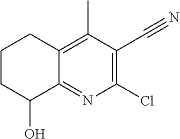

To a solution of 2-chloro-3-cyano-4-methyl-5,6,7,8-tetrahydroquinoline 1-oxide (190.0 mg, 0.70 mmol) in DCM (5 mL) was added trifluoroacetic anhydride (0.29 mL, 2.1 mmol) dropwise and the mixture was stirred at r.t. for 16 hrs. The solvent was evaporated and the residue was taken up in MeOH. K₂CO₃ was added till basic pH and the suspension was stirred at r.t. for 1 hr. The solvent was evaporated; the residue was taken up in H₂O and extracted with DCM (3×). The combined organic layers were passed through a phase separator and evaporated to dryness. The residue was purified by silica gel column chromatography using a 0-50% EtOAc/cyclohexane gradient eluent to afford the title compound (90 mg, 58%) as a white solid. ¹H NMR (400 MHz, DMSO-d₆) δ 5.59 (d, J=5.06 Hz, 1H) 4.55 (q, J=4.70 Hz, 1H) 2.68-2.79 (m, 1H) 2.54-2.64 (m, 1H) 2.45 (s, 3H) 1.68-1.99 (m, 4H). MS-ESI (m/z) calc'd for C₁₁H₁₂ClN₂O [M+H]⁺: 223.1. Found 223.0.

### Step 5: tert-Butyl 5-((2-chloro-3-cyano-4-methyl-5,6,7,8-tetrahydroquinolin-8-yl)oxy)-3-iodo-1H-indazole-1-carboxylate

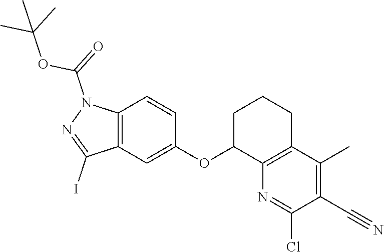 [SMILES: Cc1c(C#N)c(Cl)nc2c1CCCC2Oc1ccc2c(c1)c(I)nn2C(=O)OC(C)(C)C]

Diethyl azodicarboxylate (0.06 mL, 0.40 mmol) was added to a solution of 2-chloro-8-hydroxy-4-methyl-5,6,7,8-tetrahydroquinoline-3-carbonitrile (90.0 mg, 0.40 mmol), tert-butyl 5-hydroxy-3-iodo-1H-indazole-1-carboxylate (145.57 mg, 0.40 mmol) and triphenylphosphine (116.62 mg, 0.44 mmol) in THF (3 mL) at 0° C. The reaction mixture was stirred for 15 minutes at 0° C. and then warmed to r.t. and stirred for 2 hrs. The reaction mixture was partitioned between H₂O and EtOAc, the phases were separated, and the aqueous layer was extracted with EtOAc (2×). The combined organic phases were washed with H₂O (1×), dried over anhydrous Na₂SO₄, and evaporated to dryness. The residue was purified by silica gel column chromatography using a 0-30% EtOAc/cyclohexane gradient eluent to afford the title compound (140 mg, 61%) as a white solid. ¹H NMR (400 MHz, DMSO-d₆) δ 7.99 (d, J=9.02 Hz, 1H) 7.40 (dd, J=9.02, 2.42 Hz, 1H) 7.27 (d, J=2.42 Hz, 1H) 5.61-5.68 (m, 1H) 2.83-2.95 (m, 1H) 2.61-2.75 (m, 1H) 2.50 (3H, peak under DMSO signal) 2.15-2.25 (m, 1H) 1.86-2.05 (m, 3H) 1.65 (s, 9H). MS-ESI (m/z) calc'd for C₂₃H₂₃ClIN₄O₃ [M+H]⁺: 565.0. Found 565.2.

### Step 6: tert-Butyl 5-((2-chloro-3-cyano-4-methyl-5,6,7,8-tetrahydroquinolin-8-yl)oxy)-3-cyclopropyl-1H-indazole-1-carboxylate

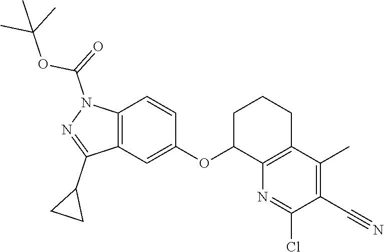 [SMILES: Cc1c(C#N)c(Cl)nc2c1CCCC2Oc1ccc2c(c1)c(C1CC1)nn2C(=O)OC(C)(C)C]

tert-Butyl 5-[(2-chloro-3-cyano-4-methyl-5,6,7,8-tetrahydroquinolin-8-yl)oxy]-3-iodoindazole-1-carboxylate (140.0 mg, 0.25 mmol), cyclopropylboronic acid (23.42 mg, 0.27 mmol) and tripotassium phosphate (102.78 mg, 0.74 mmol) were dissolved in 1,4-dioxane (3 mL) and the mixture was degassed with N₂ for 5 minutes. Pd(dppf)Cl₂ (18.14 mg, 0.02 mmol) was then added and the mixture was stirred at 100° C. under N₂ overnight. Additional Pd(dppf)Cl₂ (18.14 mg, 0.020 mmol) and cyclopropylboronic acid (23.42 mg, 0.270 mmol) were then added and the mixture was stirred at 100° C. for an additional 10 hrs. The reaction mixture was partitioned between H₂O and EtOAc, the phases were separated, and the aqueous layer was extracted with EtOAc (2×). The combined organic phases were washed with brine (1×), dried over anhydrous Na₂SO₄, and evaporated to dryness. The material was purified by silica gel column chromatography using a 0-100% EtOAc/cyclohexane gradient eluent to afford the title compound (13 mg, 11%) as a white solid. MS-ESI (m/z) calc'd for C₂₆H₂₈ClN₄O₃ [M+H]⁺: 479.2. Found 479.2.

### Step 7: 2-Chloro-8-[(3-cyclopropyl-1H-indazol-5-yl)oxy]-4-methyl-5,6,7,8-tetrahydroquinoline-3-carbonitrile, enantiomer 1 and 2

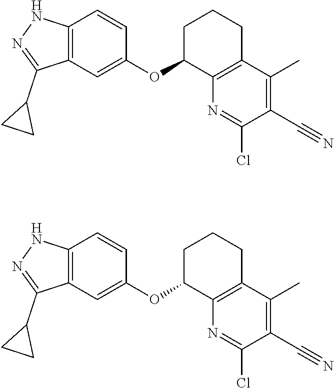 [SMILES: Cc1c(C#N)c(Cl)nc2c1CCC[C@@H]2Oc1ccc2[nH]nc(C3CC3)c2c1 ; Cc1c(C#N)c(Cl)nc2c1CCC[C@H]2Oc1ccc2[nH]nc(C3CC3)c2c1]

Prepared as described for 5-[(3-cyclopropyl-1H-indazol-5-yl)oxy]-5,6,7,8-tetrahydroquinoline-2-carbonitrile, using tert-butyl 5-[(2-chloro-3-cyano-4-methyl-5,6,7,8-tetrahydroquinolin-8-yl)oxy]-3-cyclopropylindazole-1-carboxylate in place of tert-butyl 5-[(2-cyano-5,6,7,8-tetrahydroquinolin-5-yl)oxy]-3-cyclopropyl-1H-indazole-1-carboxylate, to afford 2-chloro-8-[(3-cyclopropyl-1H-indazol-5-yl)oxy]-4-methyl-5,6,7,8-tetrahydroquinoline-3-carbonitrile, which was subjected to chiral separation using Method HE to afford 2-chloro-8-[(3-cyclopropyl-1H-indazol-5-yl)oxy]-4-methyl-5,6,7,8-tetrahydroquinoline-3-carbonitrile, enantiomer 1 (1.1 mg, 11%) as a white solid. ¹H NMR (400 MHz, MeOD) δ 7.52 (d, J=1.98 Hz, 1H) 7.39 (d, J=8.80 Hz, 1H) 7.18 (dd, J=9.02, 2.20 Hz, 1H) 5.36 (t, J=3.96 Hz, 1H) 2.89-3.01 (m, 1H) 2.64-2.77 (m, 1H) 2.58 (s, 3H) 2.36 (d, J=11.66 Hz, 1H) 2.12-2.30 (m, 2H) 1.92-2.04 (m, 2H) 1.00-1.09 (m, 4H). MS-ESI (m/z) calc'd for C₂₁C₂₀ClN₄O [M+H]⁺: 379.1. Found 379.2. A later eluting fraction was also isolated to afford 2-chloro-8-[(3-cyclopropyl-1H-indazol-5-yl)oxy]-4-methyl-5,6,7,8-tetrahydroquinoline-3-carbonitrile, enantiomer 2 (0.9 mg, 9%) as a white solid. ¹H NMR (400 MHz, MeOD) δ 7.52 (d, J=1.98 Hz, 1H) 7.39 (d, J=8.80 Hz, 1H) 7.18 (dd, J=9.02, 2.20 Hz, 1H) 5.36 (t, J=3.96 Hz, 1H) 2.89-3.01 (m, 1H) 2.64-2.77 (m, 1H) 2.58 (s, 3H) 2.36 (d, J=11.66 Hz, 1H) 2.12-2.30 (m, 2H) 1.92-2.04 (m, 2H) 1.00-1.09 (m, 4H). MS-ESI (m/z) calc'd for C₂₁H₂₀ClN₄O [M+H]⁺: 379.1. Found 379.3.

### Example 185: 2-Chloro-7-[(3-methoxy-1H-indazol-5-yl)oxy]-6,7-dihydro-5H-cyclopenta[b]pyridine-3-carbonitrile, enantiomer 1 and 2

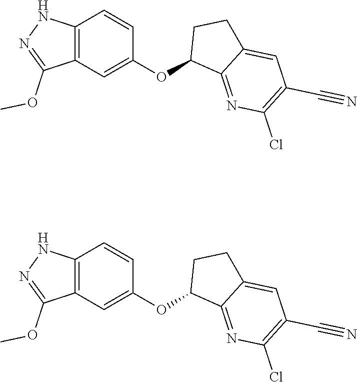 [SMILES: COc1n[nH]c2ccc(O[C@H]3CCc4cc(C#N)c(Cl)nc43)cc12 ; COc1n[nH]c2ccc(O[C@@H]3CCc4cc(C#N)c(Cl)nc43)cc12]

### Step 1: 2-Chloro-3-cyano-6,7-dihydro-5H-cyclopenta[b]pyridine 1-oxide

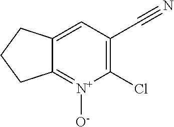 [SMILES: N#Cc1cc2c([n+]([O-])c1Cl)CCC2]

To a solution of 2-chloro-6,7-dihydro-5H-cyclopenta[b]pyridine-3-carbonitrile (2.0 g, 10.38 mmol) in trifluoroacetic acid (42 mL) was added a 30 wt. % solution of hydrogen peroxide (0.57 mL, 5.6 mmol) and the mixture was stirred at 75° C. for 18 hrs. The reaction mixture was cooled to r.t. and then concentrated under reduced pressure. Water was added and the solution was neutralized by addition of solid NaHCO₃, and then extracted with DCM (3×). The combined organic layers were passed through a phase separator and evaporated to afford the title compound (977 mg, 89%) as a beige solid. ¹H NMR (400 MHz, DMSO-d₆) δ 7.84 (s, 1H), 3.11-2.97 (m, 4H), 2.14 (p, J=7.7 Hz, 2H). ESI (m/z) calc'd for C₉H₈ClN[N⁺][O⁻] [M+H]⁺: 195.0. Found 195.0.

### Step 2: 2-Chloro-7-hydroxy-6,7-dihydro-5H-cyclopenta[b]pyridine-3-carbonitrile

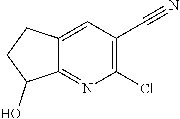 [SMILES: N#Cc1cc2c(nc1Cl)C(O)CC2]

To a solution of 2-chloro-3-cyano-6,7-dihydro-5H-cyclopenta[b]pyridine 1-oxide (2.1 g, 10.79 mmol) in DCM (53.95 mL) was added trifluoroacetic anhydride (4.5 mL, 32.37 mmol) dropwise and the mixture was stirred at 25° C. for 18 hrs. The solvent was evaporated and the residue was taken up in MeOH. Then K₂CO₃ was added until basic pH (˜8) and the suspension was stirred at 25° C. for 30 min. The solvent was evaporated, and the residue was taken up in H₂O and extracted with DCM (3×). The combined organic layers were passed through a phase separator and evaporated to dryness to afford the title compound (1.7 g, 81%) as a beige solid. ¹H NMR (400 MHz, DMSO-d₆) δ 8.33 (s, 1H), 5.79 (d, J=6.0 Hz, 2H), 5.00 (dt, J=7.5, 6.2 Hz, 2H), 2.94 (dddd, J=16.7, 8.8, 4.1, 1.1 Hz, 2H), 2.83-2.70 (m, 2H), 2.43 (dddd, J=12.5, 8.3, 7.5, 4.2 Hz, 2H), 1.86 (dddd, J=13.1, 8.8, 7.4, 6.5 Hz, 2H). MS-ESI (m/z) calc'd for C₉H₈ClN₂O [M+H]⁺: 195.0. Found 195.0.

### Step 3: tert-Butyl 5-((2-chloro-3-cyano-6,7-dihydro-5H-cyclopenta[b]pyridin-7-yl)oxy)-3-methoxy-1H-indazole-1-carboxylate

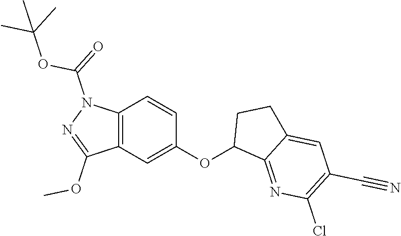 [SMILES: COc1nn(C(=O)OC(C)(C)C)c2ccc(OC3CCc4cc(C#N)c(Cl)nc43)cc12]

Diisopropyl azodicarboxylate (0.06 mL, 0.280 mmol) was added dropwise to a cooled (0° C.) solution of 2-chloro-7-hydroxy-6,7-dihydro-5H-cyclopenta[b]pyridine-3-carbonitrile (50.0 mg, 0.23 mmol), tert-butyl 5-hydroxy-3-methoxyindazole-1-carboxylate (61.79 mg, 0.23 mmol), and triphenylphosphine (122.64 mg, 0.47 mmol) in THF (4.83 mL). After 10 min the mixture was stirred at r.t. for 18 hrs. The reaction mixture was diluted with H₂O and EtOAc, the organic phase was separated, dried over Na₂SO₄, filtered and concentrated under reduced pressure. The residue was purified by silica gel column chromatography using a 0-100% EtOAc/cyclohexane gradient eluent to afford a residue (350 mg) of insufficient purity. The residue was further purified by reverse phase column chromatography on a 10 g Cis cartridge using a 2-80% MeCN/H₂O (0.1% HCOOH) gradient eluent to afford the title compound (86 mg, 83%) as a white solid. ¹H NMR (400 MHz, DMSO-d₆) δ 8.48 (s, 1H), 7.89 (d, J=9.2 Hz, 1H), 7.38 (d, J=2.5 Hz, 1H), 7.31 (dd, J=9.1, 2.5 Hz, 1H), 5.95 (dd, J=7.2, 4.8 Hz, 1H), 4.08 (s, 3H), 3.15-3.03 (m, 1H), 3.01-2.87 (m, 1H), 2.71 (tdd, J=11.5, 8.3, 4.3 Hz, 1H), 2.21-2.08 (m, 1H), 1.63 (s, 9H). MS-ESI (m/z) calc'd for C₂₂H₂₂ClN₄O₄ [M+H]⁺: 441.1. Found 441.3.

### Step 4: 2-Chloro-7-[(3-methoxy-1H-indazol-5-yl)oxy]-6,7-dihydro-5H-cyclopenta[b]pyridine-3-carbonitrile, enantiomer 1 and 2

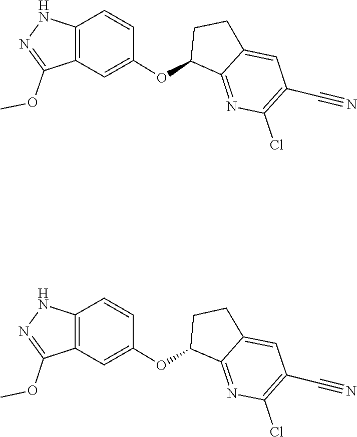 [SMILES: COc1n[nH]c2ccc(O[C@H]3CCc4cc(C#N)c(Cl)nc43)cc12 ; COc1n[nH]c2ccc(O[C@@H]3CCc4cc(C#N)c(Cl)nc43)cc12]

Prepared as described for 5-[(3-cyclopropyl-1H-indazol-5-yl)oxy]-5,6,7,8-tetrahydroquinoline-2-carbonitrile, using tert-butyl 5-[(2-chloro-3-cyano-6,7-dihydro-5H-cyclopenta[b]pyridin-7-yl)oxy]-3-methoxyindazole-1-carboxylate in place of tert-butyl 5-[(2-cyano-5,6,7,8-tetrahydroquinolin-5-yl)oxy]-3-cyclopropyl-1H-indazole-1-carboxylate, to afford 2-chloro-7-[(3-methoxy-1H-indazol-5-yl)oxy]-6,7-dihydro-5H-cyclopenta[b]pyridine-3-carbonitrile, which was subjected to chiral separation using Method HF to afford 2-chloro-7-[(3-methoxy-1H-indazol-5-yl)oxy]-6,7-dihydro-5H-cyclopenta[b]pyridine-3-carbonitrile, enantiomer 1 (15.6 mg, 23%) as a white solid. ¹H NMR (400 MHz, DMSO-d₆) δ 11.77 (s, 1H), 8.47 (s, 1H), 7.29 (d, J=9.0 Hz, 1H), 7.22 (d, J=2.4 Hz, 1H), 7.05 (dd, J=9.0, 2.4 Hz, 1H), 5.82 (dd, J=7.2, 4.8 Hz, 1H), 3.99 (s, 3H), 3.13-3.02 (m, 1H), 3.01-2.85 (m, 1H), 2.76-2.59 (m, 1H), 2.21-2.05 (m, 1H). MS-ESI (m/z) calc'd for C₁₇H₁₄ClN₄O₂ [M+H]⁺: 341.1. Found 341.2. A later eluting fraction was also isolated to afford 2-chloro-7-[(3-methoxy-1H-indazol-5-yl)oxy]-6,7-dihydro-5H-cyclopenta[b]pyridine-3-carbonitrile, enantiomer 2 (21.1 mg, 32%) as a white solid. ¹H NMR (400 MHz, DMSO-d₆) δ 11.77 (s, 1H), 8.47 (s, 1H), 7.30 (d, J=8.9 Hz, 1H), 7.23 (d, J=2.4 Hz, 1H), 7.05 (dd, J=9.0, 2.4 Hz, 1H), 5.82 (dd, J=7.2, 4.7 Hz, 1H), 3.99 (s, 3H), 3.14-3.02 (m, 1H), 3.01-2.87 (m, 1H), 2.68 (dtd, J=13.6, 8.5, 5.4 Hz, 1H), 2.14 (ddt, J=13.9, 8.8, 5.4 Hz, 1H). MS-ESI (m/z) calc'd for C₁₇H₁₄ClN₄O₂ [M+H]⁺: 341.1. Found 341.1.

### Example 186: 2-Chloro-8-[(3-methoxy-1H-indazol-5-yl)oxy]-5,6,7,8-tetrahydroquinoline-3-carbonitrile, enantiomer 1 and 2

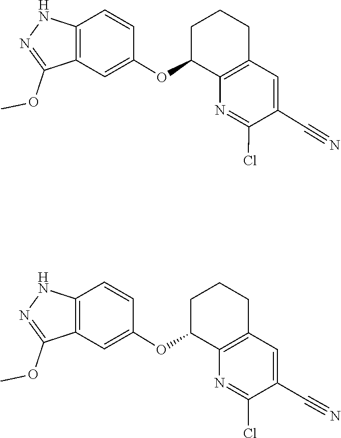 [SMILES: COc1n[nH]c2ccc(O[C@H]3CCCc4cc(C#N)c(Cl)nc43)cc12 ; COc1n[nH]c2ccc(O[C@@H]3CCCc4cc(C#N)c(Cl)nc43)cc12]

### Step 1: 2-Chloro-3-cyano-5,6,7,8-tetrahydroquinoline 1-oxide

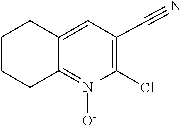 [SMILES: N#Cc1cc2c([n+]([O-])c1Cl)CCCC2]

To a solution of 2-chloro-5,6,7,8-tetrahydroquinoline-3-carbonitrile (3.0 g, 15.57 mmol) in trifluoroacetic acid (40 mL), was added a 30 wt. % solution of hydrogen peroxide in H₂O (4.77 mL, 46.72 mmol) and the mixture was stirred at 75° C. for 3 hrs. After that time an additional 3 eq of hydrogen peroxide (4.77 mL, 46.72 mmol) were added and the reaction was stirred at 75° C. overnight. Water was added and the solution was neutralized by addition of solid K₂CO₃ and then extracted with DCM (3×). The combined organic layers were passed through a phase separator and evaporated to afford the title compound (3.25 g, 100%) as a yellow solid. ¹H NMR (400 MHz, DMSO-d₆) δ 7.77 (s, 1H) 2.77 (dt, J=16.40, 6.33 Hz, 4H) 1.77-1.86 (m, 2H) 1.63-1.73 (m, 2H). MS-ESI (m/z) calc'd for C₁₀H₁₀ClN[N⁺][O⁻][M+H]⁺: 209.0. Found 209.0.

### Step 2: 2-Chloro-8-hydroxy-5,6,7,8-tetrahydroquinoline-3-carbonitrile

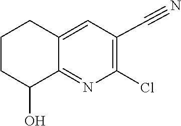 [SMILES: N#Cc1cc2c(nc1Cl)C(O)CCC2]

To a solution of 2-chloro-3-cyano-5,6,7,8-tetrahydroquinoline 1-oxide (3.25 g, 13.71 mmol) in DCM (60 mL) was added trifluoroacetic anhydride (5.72 mL, 41.12 mmol) dropwise and the mixture was stirred at 25° C. for 20 hrs. The solvent was evaporated and the residue was taken up in MeOH. K₂CO₃ was added till basic pH and the suspension was stirred at 25° C. for 2 hrs. The solvent was evaporated keeping the temperature under 40° C. and the residue was taken up in H₂O and extracted with DCM (3×). The combined organic layers were passed through a phase separator and evaporated to dryness. The residue was purified by silica gel column chromatography using a 0-40% EtOAc/cyclohexane gradient eluent to afford the title compound (2.3 g, 80%) as a white solid. ¹H NMR (400 MHz, DMSO-d₆) δ 8.24 (s, 1H) 5.61 (d, J=4.62 Hz, 1H) 4.55 (d, J=4.18 Hz, 1H) 2.64-2.86 (m, 2H) 1.67-1.93 (m, 4H). MS-ESI (m/z) calc'd for C₁₀H₁₀ClN₂O [M+H]⁺: 209.0. Found 209.0.

### Step 3: tert-Butyl 5-((2-chloro-3-cyano-6,7-dihydro-5H-cyclopenta[b]pyridin-7-yl)oxy)-3-methoxy-1H-indazole-1-carboxylate

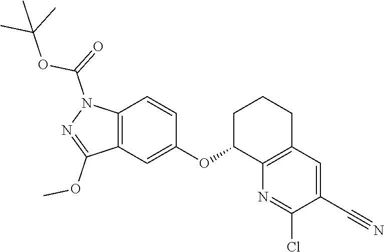 [SMILES: COc1nn(C(=O)OC(C)(C)C)c2ccc(O[C@@H]3CCCc4cc(C#N)c(Cl)nc43)cc12]

Diethyl azodicarboxylate (0.08 mL, 0.48 mmol) was added to a solution of 2-chloro-8-hydroxy-5,6,7,8-tetrahydroquinoline-3-carbonitrile (100.0 mg, 0.48 mmol), tert-butyl 5-hydroxy-3-methoxyindazole-1-carboxylate (126.67 mg, 0.48 mmol) and triphenylphosphine (138.29 mg, 0.53 mmol) in THF (4 mL) at 0° C. The reaction mixture was stirred for 15 minutes at 0° C., then warmed to r.t. and stirred for 2.5 hrs. Additional triphenylphosphine (138.29 mg, 0.53 mmol) and diethyl azodicarboxylate (0.08 mL, 0.48 mmol) were added and the mixture was stirred for 20 minutes at 25° C. The reaction mixture was partitioned between H₂O and EtOAc, the phases were separated, and the aqueous layer was extracted with EtOAc (2×). The combined organic phases were washed with brine (1×), dried over anhydrous Na₂SO₄, and evaporated to dryness. The material was purified by silica gel column chromatography using a 0-40% EtOAc/cyclohexane gradient eluent to afford the title compound (79 mg, 82%) as a white solid. ¹H NMR (400 MHz, DMSO-d₆) δ 8.40 (s, 1H) 7.90 (d, J=9.02 Hz, 1H) 7.42 (d, J=2.42 Hz, 1H) 7.32 (dd, J=9.13, 2.53 Hz, 1H) 5.58 (t, J=4.07 Hz, 1H) 4.08 (s, 3H) 2.74-2.98 (m, 2H) 1.97-2.22 (m, 2H) 1.79-1.94 (m, 2H) 1.64 (s, 9H). MS-ESI (m/z) calc'd for C₂₃H₂₄ClN₄O₄ [M+H]⁺: 455.1. Found 455.2.

### Step 4: 2-Chloro-8-[(3-methoxy-1H-indazol-5-yl)oxy]-5,6,7,8-tetrahydroquinoline-3-carbonitrile, enantiomer 1 and 2

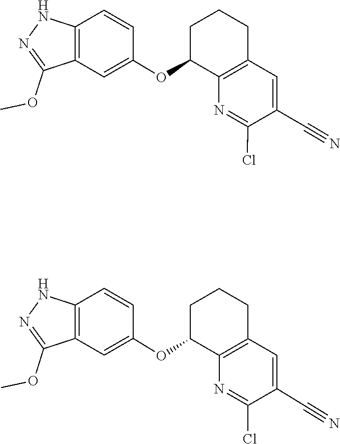 [SMILES: COc1n[nH]c2ccc(O[C@H]3CCCc4cc(C#N)c(Cl)nc43)cc12 ; COc1n[nH]c2ccc(O[C@@H]3CCCc4cc(C#N)c(Cl)nc43)cc12]

Prepared as described for 5-[(3-cyclopropyl-1H-indazol-5-yl)oxy]-5,6,7,8-tetrahydroquinoline-2-carbonitrile, using tert-butyl 5-[(2-chloro-3-cyano-5,6,7,8-tetrahydroquinolin-8-yl)oxy]-3-methoxyindazole-1-carboxylate in place of tert-butyl 5-[(2-cyano-5,6,7,8-tetrahydroquinolin-5-yl)oxy]-3-cyclopropyl-1H-indazole-1-carboxylate, to afford 2-chloro-8-[(3-methoxy-1H-indazol-5-yl)oxy]-5,6,7,8-tetrahydroquinoline-3-carbonitrile, which was subjected to chiral separation using Method HG to afford 2-chloro-8-[(3-methoxy-1H-indazol-5-yl)oxy]-5,6,7,8-tetrahydroquinoline-3-carbonitrile, enantiomer 1 (23.4 mg, 20%) as a white solid. ¹H NMR (400 MHz, DMSO-d₆) δ 11.77 (s, 1H) 8.39 (s, 1H) 7.30 (d, J=9.02 Hz, 1H) 7.25 (d, J=2.20 Hz, 1H) 7.08 (dd, J=8.91, 2.31 Hz, 1H) 5.41 (t, J=3.85 Hz, 1H) 3.99 (s, 3H) 2.87-3.00 (m, 1H) 2.72-2.84 (m, 1H) 2.13-2.25 (m, 1H) 1.75-2.03 (m, 3H). MS-ESI (m/z) calc'd for C₁₈H₁₆ClN₄O₂ [M+H]⁺: 355.1. Found 355.2. A later eluting fraction was also isolated to afford 2-chloro-8-[(3-methoxy-1H-indazol-5-yl)oxy]-5,6,7,8-tetrahydroquinoline-3-carbonitrile, enantiomer 2 (20 mg, 17%) as a white solid. ¹H NMR (400 MHz, DMSO-d₆) δ 11.77 (s, 1H) 8.39 (s, 1H) 7.30 (d, J=9.02 Hz, 1H) 7.25 (d, J=2.20 Hz, 1H) 7.08 (dd, J=8.91, 2.31 Hz, 1H) 5.41 (t, J=3.63 Hz, 1H) 3.99 (s, 3H) 2.87-3.00 (m, 1H) 2.72-2.84 (m, 1H) 2.13-2.25 (m, 1H) 1.75-2.01 (m, 3H). MS-ESI (m/z) calc'd for C₁₈H₁₆ClN₄O₂ [M+H]⁺: 355.1. Found 355.2.

### Example 187: 2-Bromo-7-[(3-methoxy-1H-indazol-5-yl)oxy]-6,7-dihydro-5H-cyclopenta[b]pyridine-3-carbonitrile, enantiomer 1 and 2

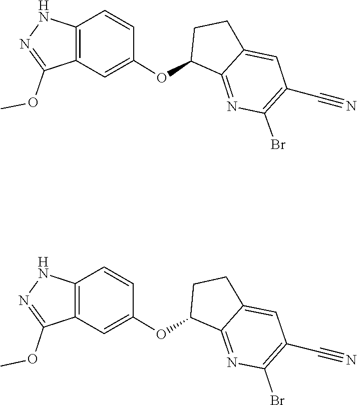 [SMILES: COc1n[nH]c2ccc(O[C@H]3CCc4cc(C#N)c(Br)nc43)cc12 ; COc1n[nH]c2ccc(O[C@@H]3CCc4cc(C#N)c(Br)nc43)cc12]

### Step 1: 2-Bromo-6,7-dihydro-5H-cyclopenta[b]pyridine-3-carbonitrile

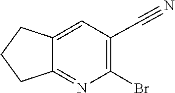 [SMILES: N#Cc1cc2c(nc1Br)CCC2]

2-Chloro-6,7-dihydro-5H-cyclopenta[b]pyridine-3-carbonitrile (925.0 mg, 5.18 mmol) was dissolved in 4.53 mL of 33 wt. % HBr in acetic acid. The reaction was stirred at 100° C. for 8 hrs. Then the reaction mixture was quenched with H₂O and extracted with EtOAc (3×). The organic phases were collected and then concentrated under reduced pressure. The residue was taken up in DCM and H₂O and filtered through a phase separator. The organic phase was concentrated under reduced pressure to afford material which was dissolved in 2.5 mL of 33 wt. % HBr in acetic acid. The reaction was then stirred at 100° C. for an additional 4 hrs. Volatiles were removed under reduced pressure to afford the title compound (1.1 g, 95%) as a beige solid. ¹H NMR (400 MHz, DMSO-d₆) δ 8.21-8.18 (m, 1H), 3.00 (t, J=7.8 Hz, 2H), 2.93-2.87 (m, 2H), 2.13-2.07 (m, 2H). ESI (m/z) calc'd for C₉H₈BrN₂: 223.0, 225.0. Found 223.0, 225.0.

### Step 2: 2-Bromo-3-cyano-6,7-dihydro-5H-cyclopenta[b]pyridine 1-oxide

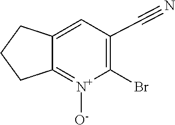 [SMILES: N#Cc1cc2c([n+]([O-])c1Br)CCC2]

To a solution of 2-bromo-6,7-dihydro-5H-cyclopenta[b]pyridine-3-carbonitrile (1.7 g, 5.18 mmol) in trifluoroacetic acid (42 mL) was added a 30 wt. % solution of hydrogen peroxide in water (0.53 mL, 5.18 mmol) and the mixture was stirred at 75° C. for 1 hr. The reaction mixture was cooled to r.t. and then concentrated under reduced pressure. Water was added and the solution was neutralized by addition of solid NaHCO₃. The mixture was then extracted with DCM (3×) and the combined organic layers were washed with brine, passed through a phase separator, and evaporated to afford the title compound (1.2 g) as a beige solid which was used without further purification. ESI (m/z) calc'd for C₉H₈BrN[N⁺][O⁻][M+H]⁺: 239.0, 241.0. Found 239.0, 241.0.

### Step 3: 2-Bromo-7-hydroxy-6,7-dihydro-5H-cyclopenta[b]pyridine-3-carbonitrile

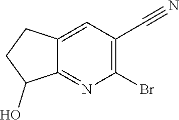 [SMILES: N#Cc1cc2c(nc1Br)C(O)CC2]

To a solution of 2-bromo-3-cyano-6,7-dihydro-5H-cyclopenta[b]pyridine 1-oxide (1.24 g, 5.18 mmol) in DCM (25.9 mL) trifluoroacetic anhydride (2.16 mL, 15.54 mmol) was added dropwise and the mixture was stirred at 25° C. for 1.5 hrs. The solvent was evaporated, the residue was taken up in MeOH, K₂CO₃ was added until basic pH (˜8) and the suspension was stirred at 25° C. for 30 min. The solvent was evaporated while keeping the temperature at 30° C. The residue was taken up in H₂O, extracted with DCM (3×) and the combined organic layers were passed through a phase separator and evaporated to dryness. The residue was purified by silica gel column chromatography using a 0-100% EtOAc/cyclohexane gradient eluent to afford the title compound (240 mg, 19% over 2 steps) as a white solid. ¹H NMR (400 MHz, CDCl₃) δ 7.81 (t, J=1.1 Hz, 1H), 5.26-5.16 (m, 1H), 3.05 (dddd, J=16.8, 9.0, 3.8, 1.0 Hz, 1H), 2.91-2.78 (m, 1H), 2.70 (d, J=3.2 Hz, 1H), 2.62 (dtd, J=13.5, 7.9, 3.8 Hz, 1H), 2.11 (dddd, J=13.8, 9.0, 7.6, 6.5 Hz, 1H). MS-ESI (m/z) calc'd for C₉H₈BrN₂O [M+H]⁺: 239.0, 241.0. Found 238.9, 240.9.

### Step 3: tert-Butyl 5-((2-bromo-3-cyano-6,7-dihydro-5H-cyclopenta[b]pyridin-7-yl)oxy)-3-methoxy-1H-indazole-1-carboxylate

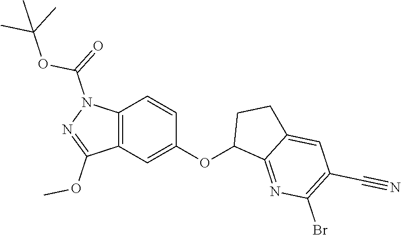 [SMILES: COc1nn(C(=O)OC(C)(C)C)c2ccc(OC3CCc4cc(C#N)c(Br)nc43)cc12]

Diisopropyl azodicarboxylate (0.06 mL, 0.30 mmol) was added dropwise to a solution of 2-bromo-7-hydroxy-6,7-dihydro-5H-cyclopenta[b]pyridine-3-carbonitrile (60.0 mg, 0.25 mmol), tert-butyl 5-hydroxy-3-methoxyindazole-1-carboxylate (66.33 mg, 0.25 mmol) and triphenylphosphine (131.66 mg, 0.50 mmol) in THF (5.309 mL) at r.t. and the mixture was stirred for 3 hrs. The reaction mixture was diluted with H₂O and EtOAc, the organic phase was separated, dried over Na₂SO₄, filtered and concentrated under reduced pressure to afford the title compound (120 mg, 98%) as a dark yellow solid which was used without further purification. MS-ESI (m/z) calc'd for C₂₂H₂₂BrN₄O₄ [M+H]⁺: 485.1, 487.1. Found 485.1, 487.2.

### Step 4: 2-Bromo-7-[(3-methoxy-1H-indazol-5-yl)oxy]-6,7-dihydro-5H-cyclopenta[b]pyridine-3-carbonitrile, enantiomer 1 and 2

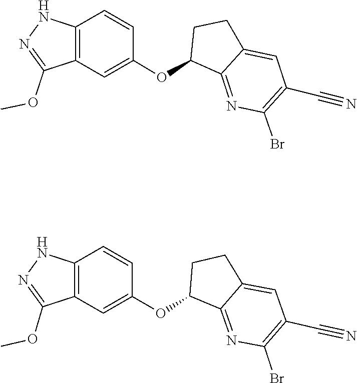 [SMILES: COc1n[nH]c2ccc(O[C@H]3CCc4cc(C#N)c(Br)nc43)cc12 ; COc1n[nH]c2ccc(O[C@@H]3CCc4cc(C#N)c(Br)nc43)cc12]

Prepared as described for 5-[(3-cyclopropyl-1H-indazol-5-yl)oxy]-5,6,7,8-tetrahydroquinoline-2-carbonitrile, using tert-butyl 5-[(2-bromo-3-cyano-6,7-dihydro-5H-cyclopenta[b]pyridin-7-yl)oxy]-3-methoxyindazole-1-carboxylate in place of tert-butyl 5-[(2-cyano-5,6,7,8-tetrahydroquinolin-5-yl)oxy]-3-cyclopropyl-1H-indazole-1-carboxylate, to afford 2-bromo-7-[(3-methoxy-1H-indazol-5-yl)oxy]-6,7-dihydro-5H-cyclopenta[b]pyridine-3-carbonitrile. This material was subjected to chiral separation using Method HH to afford 2-bromo-7-[(3-methoxy-1H-indazol-5-yl)oxy]-6,7-dihydro-5H-cyclopenta[b]pyridine-3-carbonitrile, enantiomer 1 (5.7 mg, 6%) as a white solid. ¹H NMR (400 MHz, DMSO-d₆) δ 11.78 (s, 1H), 8.41 (s, 1H), 7.30 (d, J=9.0 Hz, 1H), 7.22 (d, J=2.3 Hz, 1H), 7.05 (dd, J=9.0, 2.4 Hz, 1H), 5.82 (dd, J=7.2, 4.7 Hz, 1H), 3.99 (s, 3H), 3.15-2.98 (m, 1H), 2.96-2.85 (m, 1H), 2.74-2.59 (m, 1H), 2.19-2.04 (m, 1H). MS-ESI (m/z) calc'd for C₁₇H₁₄BrN₄O₂ [M+H]⁺: 385.0, 387.0. Found 385.1; 387.1. A later eluting fraction was also isolated to afford 2-bromo-7-[(3-methoxy-1H-indazol-5-yl)oxy]-6,7-dihydro-5H-cyclopenta[b]pyridine-3-carbonitrile, enantiomer 2 (5.4 mg, 6%) as a white solid. ¹H NMR (400 MHz, DMSO-d₆) δ 11.77 (s, 1H), 8.41 (s, 1H), 7.30 (d, J=9.0 Hz, 1H), 7.22 (d, J=2.3 Hz, 1H), 7.05 (dd, J=9.0, 2.4 Hz, 1H), 5.82 (dd, J=7.2, 4.7 Hz, 1H), 3.99 (s, 3H), 3.10-2.99 (m, 1H), 2.97-2.85 (m, 1H), 2.74-2.61 (m, 1H), 2.20-2.05 (m, 1H). MS-ESI (m/z) calc'd for C₁₇H₁₄BrN₄O₂ [M+H]⁺: 385.0, 387.0. Found 385.1, 387.1.

### Example 188: 2-Bromo-8-[(3-methoxy-1H-indazol-5-yl)oxy]-5,6,7,8-tetrahydroquinoline-3-carbonitrile, enantiomer 1 and 2

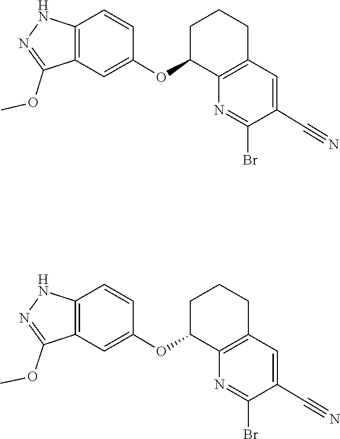 [SMILES: COc1n[nH]c2ccc(O[C@H]3CCCc4cc(C#N)c(Br)nc43)cc12 ; COc1n[nH]c2ccc(O[C@@H]3CCCc4cc(C#N)c(Br)nc43)cc12]

### Step 1: 2-Bromo-5,6,7,8-tetrahydroquinoline-3-carbonitrile

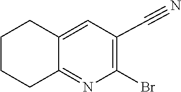 [SMILES: N#Cc1cc2c(nc1Br)CCCC2]

2-Chloro-5,6,7,8-tetrahydroquinoline-3-carbonitrile (2.0 g, 10.38 mmol) was dissolved in 1 mL of 33 wt. % HBr in acetic acid. The reaction was stirred at 100° C. for 2 hrs. Volatiles were evaporated under reduced pressure and the acetic acid was removed by co-evaporation with CH₃CN to afford the title compound (3.1 g) as a light brown solid which was used without further purification. ¹H NMR (400 MHz, DMSO-d₆) δ 8.12 (s, 1H) 2.88 (t, J=6.49 Hz, 2H) 2.69-2.76 (m, 2H) 1.68-1.90 (m, 4H). MS-ESI (m/z) calc'd for C₁₀H₁₀BrN₂ [M+H]⁺: 237.0, 239.0. Found 237.0, 239.0.

### Step 2: 2-Bromo-3-cyano-5,6,7,8-tetrahydroquinoline 1-oxide

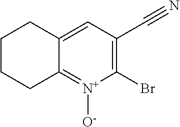 [SMILES: N#Cc1cc2c([n+]([O-])c1Br)CCCC2]

To a solution of 2-bromo-5,6,7,8-tetrahydroquinoline-3-carbonitrile (10.38 mmol) in trifluoroacetic acid (40 mL) was added a 30 wt. % solution of hydrogen peroxide in water (3.18 mL, 31.14 mmol) and the mixture was stirred at 75° C. for 3 hrs. The mixture was cooled to r.t.; H₂O was added and the solution was neutralized by addition of solid K₂CO₃. The mixture was then extracted with DCM (3×) and the combined organic layers were washed with H₂O (1×), passed through a phase separator, and evaporated to dryness to afford the title compound (2.32 g, 92%, 2 steps) as a light yellow solid. ¹H NMR (400 MHz, DMSO-d₆) δ 7.72 (s, 1H) 2.78-2.85 (m, 2H) 2.74 (t, J=5.83 Hz, 2H) 1.78-1.87 (m, 2H) 1.63-1.72 (m, 2H). MS-ESI (m/z) calc'd for C₁₀H₁₀BrN[N⁺][O⁻] [M+H]⁺: 253.0, 255.0. Found 253.0, 255.0.

### Step 3: 2-Bromo-8-hydroxy-5,6,7,8-tetrahydroquinoline-3-carbonitrile

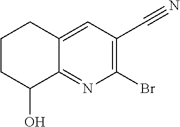 [SMILES: N#Cc1cc2c(nc1Br)C(O)CCC2]

To a solution of 2-bromo-3-cyano-5,6,7,8-tetrahydroquinoline 1-oxide (2.32 g, 9.17 mmol) in DCM (50 mL) was added trifluoroacetic anhydride (3.82 mL, 27.5 mmol) dropwise and the mixture was stirred at 25° C. for 20 hrs. The solvent was evaporated and the residue was taken up in MeOH; then K₂CO₃ was added till basic pH and the suspension was stirred at 25° C. for 30 min. The solvent was evaporated under reduced pressure. The material was partitioned between H₂O and DCM, the phases were separated, the aqueous layer was extracted with DCM (2×), and the combined organic phases were washed with H₂O (1×), dried over anhydrous Na₂SO₄ and evaporated to dryness. The residue was purified by silica gel column chromatography using a 0-50% EtOAc/cyclohexane gradient eluent to afford the title compound (395 mg, 17%) as a white solid. ¹H NMR (400 MHz, DMSO-d₆) δ 8.18 (s, 1H) 5.61 (d, J=5.28 Hz, 1H) 4.56 (q, J=4.77 Hz, 1H) 2.73-2.88 (m, 1H) 2.59-2.73 (m, 1H) 1.80-1.95 (m, 3H) 1.64-1.76 (m, 1H). MS-ESI (m/z) calc'd for C₁₀H₁₀BrN₂O [M+H]⁺: 253.0. 255.0. Found 253.0, 255.0.

### Step 4: tert-Butyl 5-((2-bromo-3-cyano-5,6,7,8-tetrahydroquinolin-8-yl)oxy)-3-methoxy-1H-indazole-1-carboxylate

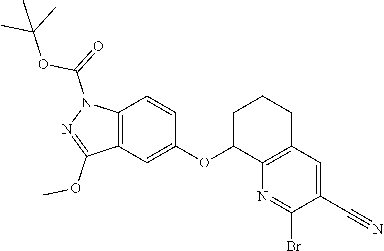 [SMILES: COc1nn(C(=O)OC(C)(C)C)c2ccc(OC3CCCc4cc(C#N)c(Br)nc43)cc12]

Diisopropyl azodicarboxylate (0.09 mL, 0.47 mmol) was added dropwise to a cooled (0° C.) solution of 2-bromo-8-hydroxy-5,6,7,8-tetrahydroquinoline-3-carbonitrile (100.0 mg, 0.40 mmol), tert-butyl 5-hydroxy-3-methoxyindazole-1-carboxylate (104.42 mg, 0.40 mmol) and triphenylphosphine (207.26 mg, 0.79 mmol) in THF (7 mL), and after 10 min the mixture was allowed to reach r.t. and stirred for 3 hrs. The reaction mixture was partitioned between water and EtOAc, the phases were separated, and the aqueous layer was extracted with EtOAc (2×). The combined organic phases were washed with brine (1×), dried over anhydrous Na₂SO₄, and evaporated to dryness. The residue was purified by silica gel column chromatography using a 0-30% EtOAc/cyclohexane gradient eluent. Selected fractions were combined and purified again by silica gel column chromatography chromatography on a 11 g NH silica gel column using a 0-30% EtOAc/cyclohexane gradient eluent to afford the title compound (92 mg, 47%) as a white solid. ¹H NMR (400 MHz, DMSO-d₆) δ 8.32 (s, 1H) 7.89 (d, J=9.24 Hz, 1H) 7.42 (d, J=2.42 Hz, 1H) 7.32 (dd, J=9.13, 2.53 Hz, 1H) 5.58 (t, J=4.07 Hz, 1H) 4.08 (s, 3H) 2.86-2.97 (m, 1H) 2.70-2.83 (m, 1H) 2.10-2.25 (m, 1H) 1.94-2.08 (m, 1H) 1.75-1.93 (m, 2H) 1.63 (s, 9H). MS-ESI (m/z) calc'd for C₂₃H₂₃BrN₄O₄ [M+H]⁺: 499.1, 501.1. Found 499.2, 501.1.

### Step 5: 2-Bromo-8-[(3-methoxy-1H-indazol-5-yl)oxy]-5,6,7,8-tetrahydroquinoline-3-carbonitrile, enantiomer 1 and 2

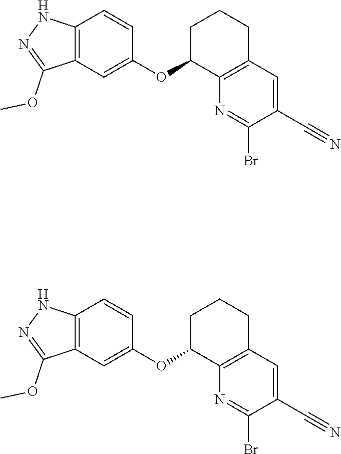 [SMILES: COc1n[nH]c2ccc(O[C@H]3CCCc4cc(C#N)c(Br)nc43)cc12 ; COc1n[nH]c2ccc(O[C@@H]3CCCc4cc(C#N)c(Br)nc43)cc12]

Prepared as described for 5-[(3-cyclopropyl-1H-indazol-5-yl)oxy]-5,6,7,8-tetrahydroquinoline-2-carbonitrile, using tert-butyl 5-[(2-bromo-3-cyano-5,6,7,8-tetrahydroquinolin-8-yl)oxy]-3-methoxyindazole-1-carboxylate in place of tert-butyl 5-[(2-cyano-5,6,7,8-tetrahydroquinolin-5-yl)oxy]-3-cyclopropyl-1H-indazole-1-carboxylate, to afford 2-bromo-8-[(3-methoxy-1H-indazol-5-yl)oxy]-5,6,7,8-tetrahydroquinoline-3-carbonitrile, which was subjected to chiral separation using Method HI to afford 2-bromo-8-[(3-methoxy-1H-indazol-5-yl)oxy]-5,6,7,8-tetrahydroquinoline-3-carbonitrile, enantiomer 1 (18.3 mg, 25%) as a white solid. ¹H NMR (400 MHz, DMSO-d₆) δ 11.77 (s, 1H) 8.31 (s, 1H) 7.30 (d, J=9.02 Hz, 1H) 7.25 (d, J=2.20 Hz, 1H) 7.08 (dd, J=8.91, 2.31 Hz, 1H) 5.42 (t, J=3.63 Hz, 1H) 3.99 (s, 3H) 2.85-2.99 (m, 1H) 2.70-2.82 (m, 1H) 2.12-2.25 (m, 1H) 1.73-2.03 (m, 3H). MS-ESI (m/z) calc'd for C₁₈H₁₆BrN₄O₂ [M+H]⁺: 399.0, 401.0. Found 399.1, 401.1. A later eluting fraction was also isolated to afford 2-bromo-8-[(3-methoxy-1H-indazol-5-yl)oxy]-5,6,7,8-tetrahydroquinoline-3-carbonitrile, enantiomer 2 (19.1 mg, 26%) as a white solid. ¹H NMR (400 MHz, DMSO-d₆) δ 11.77 (s, 1H) 8.31 (s, 1H) 7.30 (d, J=9.02 Hz, 1H) 7.25 (d, J=2.20 Hz, 1H) 7.08 (dd, J=9.02, 2.42 Hz, 1H) 5.42 (t, J=3.63 Hz, 1H) 3.99 (s, 3H) 2.85-2.98 (m, 1H) 2.69-2.83 (m, 1H) 2.13-2.24 (m, 1H) 1.74-2.02 (m, 3H). MS-ESI (m/z) calc'd for C₁₈H₁₆BrN₄O₂ [M+H]⁺: 399.0, 401.0. Found 399.2, 401.1.

### Example 189: 6-Chloro-1-[(3-methoxy-1H-indazol-5-yl)oxy]-2,3-dihydro-1H-indene-5-carbonitrile, enantiomer 1 and 2

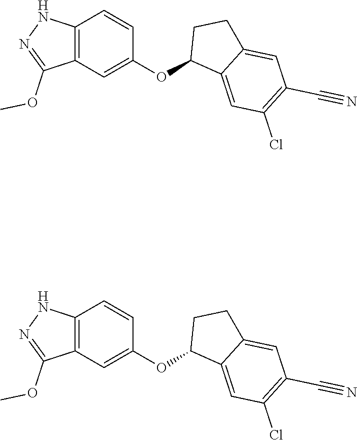 [SMILES: COc1n[nH]c2ccc(O[C@H]3CCc4cc(C#N)c(Cl)cc43)cc12 ; COc1n[nH]c2ccc(O[C@@H]3CCc4cc(C#N)c(Cl)cc43)cc12]

### Step 1: 6-Chloro-5-vinyl-2,3-dihydro-1H-inden-1-one

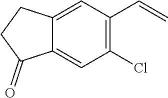

A microwave vial was charged with 5-bromo-6-chloro-2,3-dihydroinden-1-one (600.0 mg, 2.44 mmol), triphenylphosphine (19.23 mg, 0.070 mmol), tributyl(vinyl)stannane (0.86 mL, 2.93 mmol) and toluene (4 mL) under N₂. The reaction mixture was degassed with N₂ for 10 min, then Pd(PPh₃)₄ (28.24 mg, 0.020 mmol) was added and the mixture was refluxed for 18 hrs. Water (150 mL) and EtOAc (150 mL) were added. The organic phase was separated, washed with brine, dried over Na₂SO₄, filtered, and concentrated under vacuum. The residue was purified by silica gel column chromatography using a 0-60% EtOAc/cyclohexane gradient eluent to afford the title compound (365 mg, 77%). ¹H NMR (400 MHz, DMSO-d₆) δ 7.96 (s, 1H), 7.65 (s, 1H), 7.10 (dd, J=17.5, 11.1 Hz, 1H), 6.08 (dd, J=17.5, 1.0 Hz, 1H), 5.63 (dd, J=11.0, 0.9 Hz, 1H), 3.18-3.00 (m, 2H), 2.77-2.60 (m, 2H). MS-ESI (m/z) calc'd for C₁₁H₁₀ClO [M+H]⁺: 193.0. Found 193.0.

### Step 2: 6-Chloro-1-oxo-2,3-dihydro-1H-indene-5-carbaldehyde

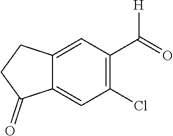 [SMILES: O=Cc1cc2c(cc1Cl)C(=O)CC2]

A solution of 6-chloro-5-vinyl-2,3-dihydro-1H-inden-1-one (360.0 mg, 1.86 mmol) in DCM (30 mL) was cooled to −78° C. A stream of ozone-enriched oxygen was introduced until a yellow color persisted. After 20 min a stream of dry nitrogen was bubbled through the reaction mixture. A solution of triphenylphosphine (585.18 mg, 2.23 mmol) in DCM (2 mL) was added dropwise and the resulting solution was allowed to warm to r.t. The reaction mixture was concentrated in vacuo to afford the title compound (360 mg) which was used without further purification. ¹H NMR (400 MHz, DMSO-d₆) δ 10.42 (s, 1H), 8.06 (s, 1H), 7.79 (s, 1H), 3.19-3.13 (m, 2H), 2.78-2.71 (m, 2H). MS-ESI (m/z) calc'd for C₁₀H₈ClO₂ [M+H]⁺: 195.0. Found 194.9.

### Step 3: (E)-6-Chloro-1-oxo-2,3-dihydro-1H-indene-5-carbaldehyde oxime

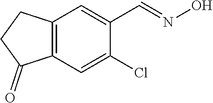 [SMILES: O=C1CCc2cc(/C=N/O)c(Cl)cc21]

To a solution of 6-chloro-1-oxo-2,3-dihydro-1H-indene-5-carbaldehyde (363.6 mg, 1.86 mmol) in MeOH (18.59 mL) were added K₂CO₃ (282.61 mg, 2.04 mmol) and hydroxylamine hydrochloride (129.17 mg, 1.86 mmol) and the mixture was stirred at 25° C. for 2 hrs. The solvent was evaporated, and the residue was taken up in H₂O and extracted with EtOAc (3×). The combined organic layers were dried over Na₂SO₄, filtered, and evaporated to afford the title compound (390 mg) as a brown oil which was used without further purification. MS-ESI (m/z) calc'd for C₁₀H₈ClNO₂ [M+H]⁺: 210.0. Found 210.0.

### Step 3: 6-Chloro-1-oxo-2,3-dihydro-1H-indene-5-carbonitrile

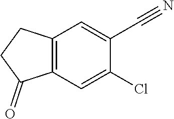 [SMILES: N#Cc1cc2c(cc1Cl)C(=O)CC2]

To a solution of (E)-6-chloro-1-oxo-2,3-dihydro-1H-indene-5-carbaldehyde oxime (389.7 mg, 1.86 mmol) in DCM (12.63 mL) were added triethylamine (518.22 uL, 3.72 mmol) and mesyl chloride (158.49 uL, 2.04 mmol) and the mixture was stirred at 25° C. for 18 hrs. The solvent was evaporated under reduced pressure and the residue was taken up in DCM and H₂O and filtered through a phase separator and concentrated under reduced pressure. The residue was purified by silica gel column chromatography using a 0-100% EtOAc/cyclohexane gradient eluent to afford the title compound (25 mg, 7%) as a beige solid. ¹H NMR (400 MHz, DMSO-d₆) δ 8.29 (s, 1H), 7.91 (s, 1H), 3.17-3.10 (m, 2H), 2.78-2.72 (m, 2H). MS-ESI (m/z) calc'd for C₁₀H₇ClNO [M+H]⁺: 192.0. Found 192.1.

### Step 4: 6-Chloro-1-hydroxy-2,3-dihydro-1H-indene-5-carbonitrile

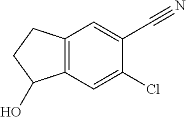 [SMILES: N#Cc1cc2c(cc1Cl)C(O)CC2]

To a solution of 6-chloro-1-oxo-2,3-dihydro-1H-indene-5-carbonitrile (24.0 mg, 0.13 mmol) in MeOH (2 mL) was added sodium borohydride (4.74 mg, 0.13 mmol) at 0° C. The resulting mixture was allowed to reach r.t. and stirred for 1 hr. The reaction mixture was quenched with saturated aqueous NaHCO₃ (10 mL) and diluted with H₂O (50 mL) and DCM (50 mL). The organic layer was washed with brine, dried over Na₂SO₄, filtered, and concentrated to afford the title compound (24 mg, 99%) which was used without further purification. ¹H NMR (400 MHz, CDCl₃) δ 7.53 (d, J=3.5 Hz, 2H), 5.29-5.22 (m, 1H), 3.08-2.97 (m, 1H), 2.82 (dt, J=15.9, 7.8 Hz, 1H), 2.64-2.53 (m, 1H), 2.06-1.94 (m, 1H). MS-ESI (m/z) calc'd for C₁₀H₉ClNO [M+H]⁺: 194.0. Found 193.9.

### Step 5: tert-Butyl 5-bromo-3-methoxy-1H-indazole-1-carboxylate

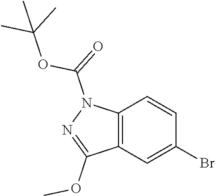 [SMILES: COc1nn(C(=O)OC(C)(C)C)c2ccc(Br)cc12]

Di-tert-butyl dicarbonate (1.52 mL, 6.61 mmol) was added to a solution of 5-bromo-3-methoxy-1H-indazole (1.0 g, 4.4 mmol) and triethylamine (0.92 mL, 6.61 mmol) in THF (20 mL) and the mixture was stirred at r.t. for 3 hrs. An additional 1.0 eq of di-tert-butyl dicarbonate (1.52 mL, 6.61 mmol) was then added and the reaction was stirred at 25° C. overnight. The reaction mixture was partitioned between H₂O and EtOAc, the phases were separated, and the aqueous layer was extracted with EtOAc (2×). The combined organic phases were washed with brine (1×), dried over anhydrous Na₂SO₄, and evaporated to dryness. The material was purified by silica gel column chromatography using a 0-10% EtOAc/cyclohexane gradient eluent to afford the title compound (1.3 g, 90%) as a white solid. ¹H NMR (400 MHz, DMSO-d₆) δ 7.89-7.97 (m, 2H) 7.77 (dd, J=8.80, 1.98 Hz, 1H) 4.08 (s, 3H) 1.61-1.66 (m, 9H). MS-ESI (m/z) calc'd for C₁₃H₁₆BrN₂O₃ [M+H]⁺: 327.0, 329.0. Found 327.1, 329.2.

### Step 6: tert-Butyl 3-methoxy-5-(4,4,5,5-tetramethyl-1,3,2-dioxaborolan-2-yl)-1H-indazole-1-carboxylate

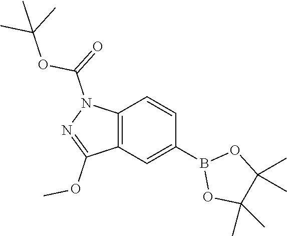 [SMILES: COc1nn(C(=O)OC(C)(C)C)c2ccc(B3OC(C)(C)C(C)(C)O3)cc12]

To a solution of tert-butyl 5-bromo-3-methoxyindazole-1-carboxylate (1.3 g, 3.97 mmol) in 1,4-dioxane (20 mL) were added Pd(dppf)Cl₂ (0.29 g, 0.40 mmol), 4,4,5,5-tetramethyl-2-(4,4,5,5-tetramethyl-1,3,2-dioxaborolan-2-yl)-1,3,2-dioxaborolane (2.02 g, 7.95 mmol) and KOAc (0.78 g, 7.95 mmol). The reaction mixture was stirred at 80° C. for 4 hrs. An additional 0.05 eq of Pd(dppf)Cl₂ (0.29 g, 0.40 mmol) was then added and the mixture was stirred overnight at 80° C. The mixture was filtered through Celite, washing with EtOAc. The filtrate was concentrated under reduced pressure to afford the title compound (3.49 g) as a black solid which was used without further purification. ¹H NMR (400 MHz, DMSO-d₆) δ 8.01 (d, J=8.58 Hz, 1H) 7.96 (s, 1H) 7.86 (dd, J=8.47, 0.99 Hz, 1H) 4.08 (s, 3H) 1.59-1.65 (m, 9H) 1.28-1.33 (m, 12H). MS-ESI (m/z) calc'd for MS-ESI (m/z) calc'd for C₁₉H₂₈BN₂O₅ [M+H]⁺: 375.2. Found 375.3.

### Step 7: tert-Butyl 5-hydroxy-3-methoxy-1H-indazole-1-carboxylate

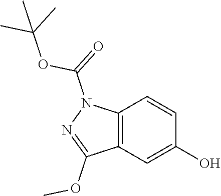 [SMILES: COc1nn(C(=O)OC(C)(C)C)c2ccc(O)cc12]

To a solution of tert-butyl 3-methoxy-5-(4,4,5,5-tetramethyl-1,3,2-dioxaborolan-2-yl)indazole-1-carboxylate (3.49 g, 3.937 mmol) in MeOH (15 mL) was added a 30 wt. % solution of hydrogen peroxide in water (2.01 mL, 19.69 mmol) and the mixture was stirred at r.t. for 1 hr. The reaction was quenched by addition of saturated aqueous Na₂SO₃ and the solvent was evaporated. The mixture was partitioned between H₂O and EtOAc, the phases were separated, and the aqueous layer was extracted with EtOAc (3×). The combined organic phases were washed with brine (1×), dried over anhydrous Na₂SO₄, filtered, and evaporated to dryness. The residue was purified by silica gel column chromatography using a 0-40% EtOAc/cyclohexane gradient eluent to afford the title compound (790 mg, 76%), as a white solid. ¹H NMR (400 MHz, DMSO-d₆) δ 9.69 (br. s., 1H) 7.78 (d, J=9.02 Hz, 1H) 7.08 (dd, J=9.02, 2.42 Hz, 1H) 6.90 (d, J=2.42 Hz, 1H) 4.01-4.06 (m, 3H) 1.57-1.64 (m, 9H). MS-ESI (m/z) calc'd for C₁₃H₁₇N₂O₄ [M+H]⁺: 265.1. Found 265.1.

### Step 8: tert-Butyl 5-((6-chloro-5-cyano-2,3-dihydro-1H-inden-1-yl)oxy)-3-methoxy-1H-indazole-1-carboxylate

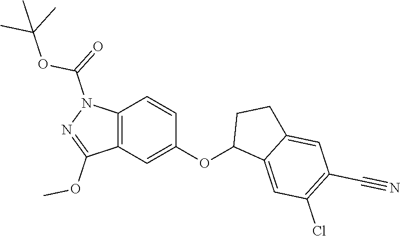 [SMILES: COc1nn(C(=O)OC(C)(C)C)c2ccc(OC3CCc4cc(C#N)c(Cl)cc43)cc12]

Diisopropyl azodicarboxylate (0.03 mL, 0.140 mmol) was added dropwise to a cooled solution of 6-chloro-1-hydroxy-2,3-dihydro-1H-indene-5-carbonitrile (24.0 mg, 0.12 mmol), tert-butyl 5-hydroxy-3-methoxyindazole-1-carboxylate (31.12 mg, 0.12 mmol) and triphenylphosphine (61.77 mg, 0.24 mmol) in THF (1.8 mL) and the mixture was stirred for 2 hrs. The reaction mixture was then diluted with H₂O and EtOAc. The organic phase was separated, dried over Na₂SO₄, filtered, and concentrated under reduced pressure. The material was purified by silica gel column chromatography using a 0-100% EtOAc/cyclohexane gradient eluent to afford the title compound (12 mg, 23%) as a pale yellow solid. ¹H NMR (400 MHz, CDCl₃) δ 7.90 (s, 1H), 7.60 (s, 1H), 7.55 (s, 1H), 7.16 (d, J=8.3 Hz, 2H), 5.79-5.70 (m, 1H), 4.18 (s, 3H), 3.21-3.06 (m, 1H), 3.03-2.86 (m, 1H), 2.77-2.62 (m, 1H), 2.36-2.21 (m, 1H), 1.70 (s, 9H). MS-ESI (m/z) calc'd for C₂₃H₂₃ClN₃O₄ [M+H]⁺: 440.1. Found 440.2.

### Step 9: 6-Chloro-1-[(3-methoxy-1H-indazol-5-yl)oxy]-2,3-dihydro-1H-indene-5-carbonitrile, enantiomer 1 and 2

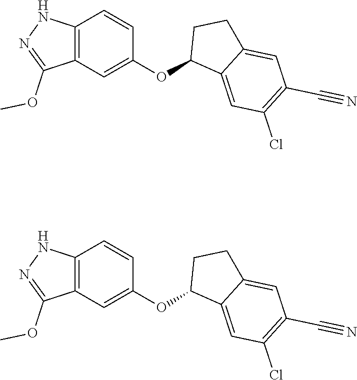 [SMILES: COc1n[nH]c2ccc(O[C@H]3CCc4cc(C#N)c(Cl)cc43)cc12 ; COc1n[nH]c2ccc(O[C@@H]3CCc4cc(C#N)c(Cl)cc43)cc12]

Prepared as described for 5-[(3-cyclopropyl-1H-indazol-5-yl)oxy]-5,6,7,8-tetrahydroquinoline-2-carbonitrile, using tert-butyl 5-[(6-chloro-5-cyano-2,3-dihydro-1H-inden-1-yl)oxy]-3-methoxyindazole-1-carboxylate in place of tert-butyl 5-[(2-cyano-5,6,7,8-tetrahydroquinolin-5-yl)oxy]-3-cyclopropyl-1H-indazole-1-carboxylate, to afford 6-chloro-1-[(3-methoxy-1H-indazol-5-yl)oxy]-2,3-dihydro-1H-indene-5-carbonitrile, which was subjected to chiral separation using Method HJ to afford 6-chloro-1-[(3-methoxy-1H-indazol-5-yl)oxy]-2,3-dihydro-1H-indene-5-carbonitrile, enantiomer 1 (2.1 mg, 23%) as a white solid. ¹H NMR (400 MHz, MeOD) δ 7.75 (s, 1H), 7.59 (s, 1H), 7.29 (dd, J=9.0, 0.7 Hz, 1H), 7.19 (d, J=2.3 Hz, 1H), 7.09 (dd, J=9.0, 2.4 Hz, 1H), 5.87-5.79 (m, 1H), 4.06 (s, 3H), 3.19-3.07 (m, 1H), 2.98 (dt, J=16.1, 7.4 Hz, 1H), 2.79-2.58 (m, 1H), 2.30-2.14 (m, 1H). MS-ESI (m/z) calc'd for C₁₈H₁₅ClN₃O₂ [M+H]⁺: 340.1. Found 340.1. A later eluting fraction was also isolated to afford 6-chloro-1-[(3-methoxy-1H-indazol-5-yl)oxy]-2,3-dihydro-1H-indene-5-carbonitrile, enantiomer 2 (1.9 mg, 20%) as a white solid. ¹H NMR (400 MHz, MeOD) δ 7.75 (s, 1H), 7.59 (s, 1H), 7.29 (dd, J=9.0, 0.8 Hz, 1H), 7.19 (d, J=2.3 Hz, 1H), 7.09 (dd, J=9.0, 2.4 Hz, 1H), 5.87-5.79 (m, 1H), 4.06 (s, 3H), 3.13 (ddd, J=13.7, 8.7, 4.4 Hz, 1H), 2.98 (dt, J=16.1, 7.4 Hz, 1H), 2.69 (dddd, J=13.4, 8.4, 6.8, 5.0 Hz, 1H), 2.22 (dddd, J=13.7, 8.7, 6.5, 5.2 Hz, 1H). MS-ESI (m/z) calc'd for C₁₈H₁₅ClN₃O₂ [M+H]⁺: 340.1. Found 340.1.

### Example 190: 8-[[6-Methyl-3-(1,3-oxazol-5-yl)-1H-indazol-5-yl]amino]-5,6,7,8-tetrahydroquinoline-3-carbonitrile, enantiomer 1 and 2

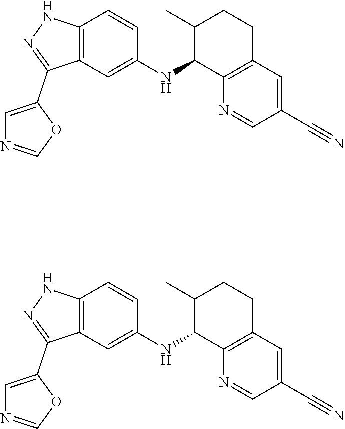 [SMILES: CC1CCc2cc(C#N)cnc2[C@H]1Nc1ccc2[nH]nc(-c3cnco3)c2c1 ; CC1CCc2cc(C#N)cnc2[C@@H]1Nc1ccc2[nH]nc(-c3cnco3)c2c1]

### Step 1: 3-Bromo-6-methyl-1H-indazol-5-amine

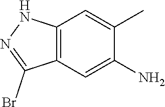

A mixture of 3-bromo-6-methyl-5-nitro-1H-indazole (500.0 mg, 1.95 mmol), NH₄Cl (0.11 g, 2.15 mmol) and iron powder (0.44 g, 7.81 mmol) in EtOH (8 mL) and H₂O (8 mL) was stirred at 80° C. for 2 hrs. The solids were removed by filtration through Celite and the solid was washed with EtOH. Volatiles were removed under vacuum to give a residue that was dissolved in EtOAc. Water was added and the two phases were separated. The aqueous layer was extracted with EtOAc (2×) and the combined organic layers were washed with H₂O (1×), dried over anhydrous Na₂SO₄, and the solvent was removed under reduced pressure to afford the title compound (390 mg, 88%) as a light brown solid. ¹H NMR (400 MHz, DMSO-d₆) δ 12.78 (1H, s) 7.17 (1H, s) 6.64 (1H, s) 4.77 (2H, s) 2.21 (3H, s). MS-ESI (m/z) calc'd for C₈H₉BrN₃ [M+H]⁺: 226.0, 228.0. Found 226.0, 227.9.

### Step 2: 8-((3-Bromo-1H-indazol-5-yl)amino)-7-methyl-5,6,7,8-tetrahydroquinoline-3-carbonitrile

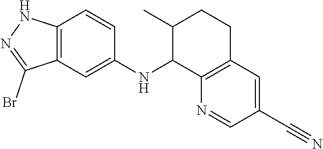 [SMILES: CC1CCc2cc(C#N)cnc2C1Nc1ccc2[nH]nc(Br)c2c1]

To a stirred solution of 3-bromo-6-methyl-1H-indazol-5-amine (388.64 mg, 1.72 mmol) and 8-oxo-5,6,7,8-tetrahydroquinoline-3-carbonitrile (200.0 mg, 1.16 mmol) in 1,4-dioxane (8 mL) was added 4-methylbenzenesulfonic acid hydrate (22.1 mg, 0.12 mmol) and the mixture was stirred at 100° C. for 4 hrs. The reaction was cooled to 40° C. and sodium triacetoxyborohydride (669.97 mg, 3.48 mmol) was added portionwise over 3 hrs and the mixture was then stirred overnight at 40° C. The reaction mixture was partitioned between H₂O and EtOAc, and the phases were separated. The aqueous layer was extracted with EtOAc (3×) and the combined organic phases were washed with brine (2×), dried over anhydrous Na₂SO₄, and evaporated to dryness. The residue was purified by silica gel chromatography on a 25 g column using as 0-30% EtOAc/cyclohexane gradient eluent to give material of insufficient purity. It was purified again by reverse phase chromatography on a 30 g C18 column using a 5-70% MeCN/H₂O (0.1% formic acid) to afford the title compound (135 mg, 30%) as a yellow solid. ¹H NMR (400 MHz, DMSO-d₆) δ 12.91 (1H, s) 8.83 (1H, d, J=1.76 Hz) 8.15 (1H, d, J=1.76 Hz) 7.29 (1H, s) 6.64 (1H, s) 5.21 (1H, d, J=5.50 Hz) 4.59-4.76 (1H, m) 2.90 (2H, br. s.) 2.19-2.33 (4H, m) 1.79-2.05 (3H, m). MS-ESI (m/z) calc'd for C₁₈H₁₇BrN₅ [M+H]⁺: 382.1, 384.1. Found 382.1, 384.1.

### Step 3: 8-[[6-Methyl-3-(1,3-oxazol-5-yl)-1H-indazol-5-yl]amino]-5,6,7,8-tetrahydroquinoline-3-carbonitrile, enantiomer 1 and 2

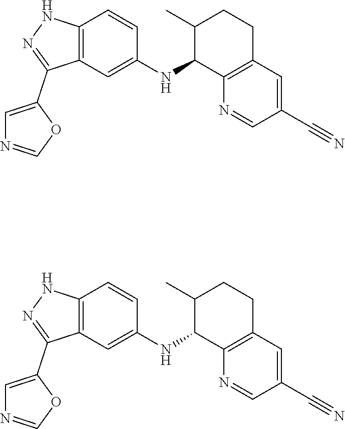 [SMILES: CC1CCc2cc(C#N)cnc2[C@H]1Nc1ccc2[nH]nc(-c3cnco3)c2c1 ; CC1CCc2cc(C#N)cnc2[C@@H]1Nc1ccc2[nH]nc(-c3cnco3)c2c1]

8-[(3-Bromo-6-methyl-1H-indazol-5-yl)amino]-5,6,7,8-tetrahydroquinoline-3-carbonitrile (60.0 mg, 0.16 mmol), oxazole-5-boronic acid pinacol ester (33.67 mg, 0.17 mmol) and KOAc (27.73 mg, 0.28 mmol) were dissolved in 1,4-dioxane (2 mL) and water (0.40 mL) in a microwave vial. The mixture was degassed with N₂ for 5 minutes and Pd(amphos)Cl₂ (11.11 mg, 0.02 mmol) was added. The mixture was sealed and heated at 100° C. using microwave irradiation under N₂ for 30 min. The reaction mixture was partitioned between H₂O and EtOAc, and the phases were separated. The aqueous layer was extracted with EtOAc (2×) and the combined organic phases were washed with H₂O (1×), dried over anhydrous Na₂SO₄, and evaporated to dryness. The residue was purified by silica gel chromatography on a 10 g column, using a 0-100% EtOAc/cyclohexane gradient eluent. The purest fractions were combined and purified again by column chromatography on a 6 g NH silica gel column, using a 50-100% EtOAc/cyclohexane gradient eluent to afford 8-[[6-methyl-3-(1,3-oxazol-5-yl)-1H-indazol-5-yl]amino]-5,6,7,8-tetrahydroquinoline-3-carbonitrile, which was subjected to chiral separation using Method HK to afford 8-[[6-methyl-3-(1,3-oxazol-5-yl)-1H-indazol-5-yl]amino]-5,6,7,8-tetrahydroquinoline-3-carbonitrile, enantiomer 1 (15.6 mg, 27%) as a yellow solid. ¹H NMR (400 MHz, DMSO-d₆) δ 13.00 (s, 1H), 8.82 (d, J=2.1 Hz, 1H), 8.46 (s, 1H), 8.15 (d, J=2.0 Hz, 1H), 7.66 (s, 1H), 7.32 (s, 1H), 7.09 (s, 1H), 5.18 (d, J=5.7 Hz, 1H), 4.86-4.76 (m, 1H), 2.98-2.85 (m, 2H), 2.31-2.18 (m, 4H), 2.02-1.86 (m, 3H). MS-ESI (m/z) calc'd for C₂₁H₁₉N₆O [M+H]⁺: 371.2. Found 371.2. A later eluting fraction was also isolated to afford 8-[[6-methyl-3-(1,3-oxazol-5-yl)-1H-indazol-5-yl]amino]-5,6,7,8-tetrahydroquinoline-3-carbonitrile, enantiomer 2 (15.4 mg, 26%) as a yellow solid. ¹H NMR (400 MHz, DMSO-d₆) δ 13.01 (s, 1H), 8.82 (d, J=2.1 Hz, 1H), 8.46 (s, 1H), 8.15 (d, J=2.1 Hz, 1H), 7.66 (s, 1H), 7.32 (s, 1H), 7.09 (s, 1H), 5.18 (d, J=5.7 Hz, 1H), 4.85-4.75 (m, 1H), 3.00-2.81 (m, 2H), 2.29-2.22 (m, 4H), 1.99-1.89 (m, 3H). MS-ESI (m/z) calc'd for C₂₁H₁₉N₆O [M+H]⁺: 371.2. Found 371.2.

### Example 191: 4-((3-(Oxazol-5-yl)-1H-indazol-5-yl)amino)chromane-7-carbonitrile, enantiomer 1 and 2

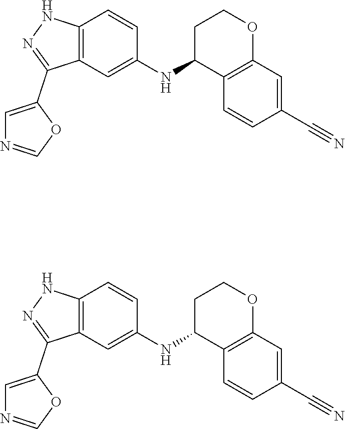 [SMILES: N#Cc1ccc2c(c1)OCC[C@@H]2Nc1ccc2[nH]nc(-c3cnco3)c2c1 ; N#Cc1ccc2c(c1)OCC[C@H]2Nc1ccc2[nH]nc(-c3cnco3)c2c1]

### Step 1: 4-Hydroxychromane-7-carbonitrile

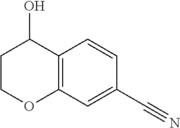 [SMILES: N#Cc1ccc2c(c1)OCCC2O]

To a solution of 4-oxo-3,4-dihydro-2H-1-benzopyran-7-carbonitrile (173.1 mg, 1.00 mmol) in MeOH (10.0 mL) was added sodium borohydride (75.6 mg, 2.00 mmol) and the mixture was stirred at 25° C. for 30 minutes. The solvent was evaporated to dryness, the residue was taken up in H₂O and extracted with DCM (3×), the combined organic layers were passed through a phase separator and evaporated to afford the title compound (175.1 mg, 100%) as a white solid. ¹H NMR (400 MHz, DMSO-d₆) δ 7.47 (d, J=7.9 Hz, 1H), 7.27 (dd, J=7.9, 1.7 Hz, 1H), 7.19 (d, J=1.6 Hz, 1H), 5.80 (d, J=5.4 Hz, 1H), 4.66 (q, J=5.3 Hz, 1H), 4.29-4.13 (m, 2H), 2.10-1.97 (m, 1H), 1.93-1.81 (m, 1H). MS-ESI (m/z) calc'd for C₁₀H₁₀NO₂ [M+H]⁺: 176.0. Found 175.9.

### Step 2: N-(7-Cyanochroman-4-yl)-2-nitro-N-(3-(oxazol-5-yl)-1-((2-(trimethylsilyl)ethoxy)methyl)-1H-indazol-5-yl)benzenesulfonamide

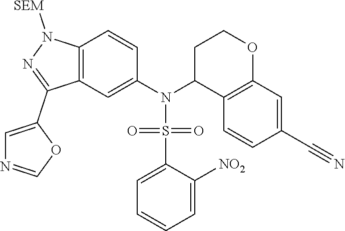 [SMILES: C[Si](C)(C)CCOCn1nc(-c2cnco2)c2cc(N(C3CCOc4cc(C#N)ccc43)S(=O)(=O)c3ccccc3[N+](=O)[O-])ccc21]

To a solution of 4-hydroxychromane-7-carbonitrile (175.1 mg, 1.00 mmol), 2-nitro-N-[3-(1,3-oxazol-5-yl)-1-(2-trimethylsilylethoxymethyl)indazol-5-yl]benzenesulfonamide (515.6 mg, 1.00 mmol) and triphenylphosphine (524.5 mg, 2.00 mmol) in THF (10.0 mL), was added dropwise diethyl azodicarboxylate (315.0 μL, 2.00 mmol) and the mixture was stirred at 25° C. for 15 hrs. The solvent was evaporated to dryness. The residue was purified by chromatography on an 11 g NH column using a 0-50% EtOAc/cyclohexane gradient eluent to afford the title compound (672 mg, 100%) as an orange solid. ¹H NMR (400 MHz, DMSO-d₆) δ 8.56-6.85 (m, 12H), 5.77 (s, 2H), 4.29-3.76 (m, 3H), 3.65-3.48 (m, 2H), 1.29-1.00 (m, 2H), 0.87-0.71 (m, 2H), −0.16 (s, 9H). MS-ESI (m/z) calc'd for C₃₂H₃₃N₆O₇SiS [M+H]⁺: 673.1. Found 672.9.

### Step 3: 4-((3-(Oxazol-5-yl)-1-((2-(trimethylsilyl)ethoxy)methyl)-1H-indazol-5-yl)amino)chromane-7-carbonitrile

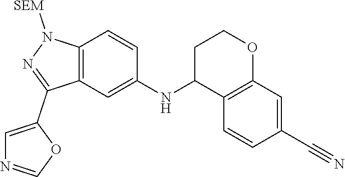 [SMILES: C[Si](C)(C)CCOCn1nc(-c2cnco2)c2cc(NC3CCOc4cc(C#N)ccc43)ccc21]

To a solution of N-(7-cyano-3,4-dihydro-2H-1-benzopyran-4-yl)-2-nitro-N-[3-(1,3-oxazol-5-yl)-1-{[2-(trimethylsilyl)ethoxy]methyl}-1H-indazol-5-yl]benzene-1-sulfonamide (672.7 mg, 1.00 mmol) in DMF (10.0 mL) were added K₂CO₃ (552.8 mg, 4.00 mmol) and benzenethiol (30.7 μL, 3.00 mmol) and the mixture was stirred at 25° C. for 1 hr. Water was added and the mixture was extracted with EtOAc (3×). The combined organic layers were evaporated to afford a yellow oil which was passed through an SCX cartridge (20 g) to afford the title compound (402 mg, 82%) as a yellow oil. ¹H NMR (400 MHz, DMSO-d₆) δ 8.53-8.49 (m, 1H), 7.85-6.68 (m, 7H), 6.21 (d, J=9.0 Hz, 1H), 5.70 (d, J=8.3 Hz, 2H), 4.97 (dt, J=9.6, 5.3 Hz, 1H), 4.42-4.24 (m, 2H), 3.64-3.47 (m, 2H), 2.22-2.08 (m, 1H), 2.09-1.95 (m, 1H), 0.90-0.73 (m, 2H), −0.07-−0.12 (m, 9H). MS-ESI (m/z) calc'd for C₂₆H₃₀N₅O₃Si [M+H]⁺: 488.2. Found 488.1.

### Step 4: rac-4-((3-(Oxazol-5-yl)-1H-indazol-5-yl)amino)chromane-7-carbonitrile

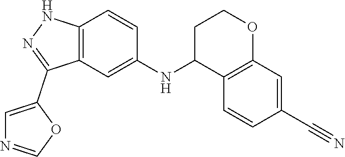 [SMILES: N#Cc1ccc2c(c1)OCCC2Nc1ccc2[nH]nc(-c3cnco3)c2c1]

Prepared as described for 1-methoxy-5-{[3-(1,3-oxazol-5-yl)-1H-indazol-5-yl]amino}-5,6,7,8-tetrahydronaphthalene-2-carbonitrile using 4-((3-(oxazol-5-yl)-1-((2-(trimethylsilyl)ethoxy)methyl)-1H-indazol-5-yl)amino)chromane-7-carbonitrile in place of 1-methoxy-5-{[3-(1,3-oxazol-5-yl)-1-{[2-(trimethylsilyl)ethoxy]methyl}-1H-indazol-5-yl]amino}-5,6,7,8-tetrahydronaphthalene-2-carbonitrile, to afford the title compound (121 mg, 41%).

### Step 5: 4-((3-(Oxazol-5-yl)-1H-indazol-5-yl)amino)chromane-7-carbonitrile, enantiomer 1 and 2

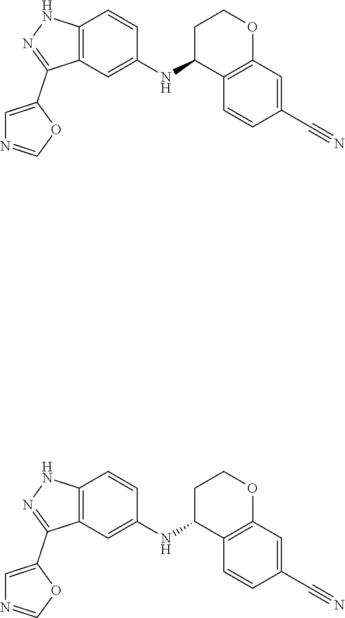 [SMILES: N#Cc1ccc2c(c1)OCC[C@@H]2Nc1ccc2[nH]nc(-c3cnco3)c2c1 ; N#Cc1ccc2c(c1)OCC[C@H]2Nc1ccc2[nH]nc(-c3cnco3)c2c1]

4-((3-(Oxazol-5-yl)-1H-indazol-5-yl)amino)chromane-7-carbonitrile was subjected to chiral separation using Method CW to afford 4-((3-(oxazol-5-yl)-1H-indazol-5-yl)amino)chromane-7-carbonitrile, enantiomer 1 (32.4 mg, 11%) as a white solid. ¹H NMR (400 MHz, DMSO-d₆) δ 13.12 (s, 1H), 8.46 (s, 1H), 7.69 (s, 1H), 7.50 (d, J=7.8 Hz, 1H), 7.39 (d, J=8.9 Hz, 1H), 7.32 (d, J=1.5 Hz, 1H), 7.33-7.27 (m, 1H), 7.10 (d, J=2.1 Hz, 1H), 7.01 (dd, J=9.0, 2.1 Hz, 1H), 6.10 (d, J=9.0 Hz, 1H), 4.94 (dt, J=9.4, 5.2 Hz, 1H), 4.44-4.22 (m, 2H), 2.13 (ddt, J=12.2, 8.3, 3.6 Hz, 1H), 2.03 (ddt, J=10.9, 8.1, 3.7 Hz, 1H). MS-ESI (m/z) calc'd for C₂₀H₁₆N₅O₂ [M+H]⁺: 358.1. Found 358.2. A later eluting fraction was also isolated to afford 4-{[3-(1,3-oxazol-5-yl)-1H-indazol-5-yl]amino}-3,4-dihydro-2H-1-benzopyran-7-carbonitrile, enantiomer 2 (30.3 mg, 10%) as a white solid. ¹H NMR (400 MHz, DMSO-d₆) δ 13.12 (s, 1H), 8.46 (s, 1H), 7.69 (s, 1H), 7.50 (d, J=7.8 Hz, 1H), 7.39 (d, J=8.9 Hz, 1H), 7.32 (d, J=1.6 Hz, 1H), 7.31-7.28 (m, 1H), 7.10 (d, J=2.0 Hz, 1H), 7.01 (dd, J=9.0, 2.1 Hz, 1H), 6.10 (d, J=9.0 Hz, 1H), 4.94 (dt, J=9.6, 5.3 Hz, 1H), 4.44-4.22 (m, 2H), 2.13 (ddt, J=12.1, 8.3, 3.7 Hz, 1H), 2.08-1.96 (m, 1H). MS-ESI (m/z) calc'd for C₂₀H₁₆N₅O₂ [M+H]⁺: 358.1. Found 358.2.

### Example 192: 4-((3-(Isoxazol-4-yl)-1H-indazol-5-yl)amino)chromane-7-carbonitrile, enantiomer 1 and 2

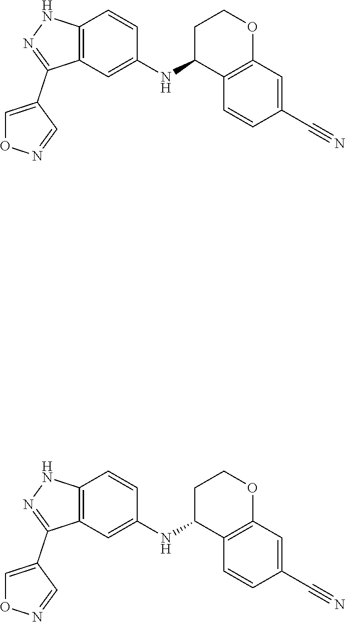 [SMILES: N#Cc1ccc2c(c1)OCC[C@@H]2Nc1ccc2[nH]nc(-c3cnoc3)c2c1 ; N#Cc1ccc2c(c1)OCC[C@H]2Nc1ccc2[nH]nc(-c3cnoc3)c2c1]

### Step 1: 4-((3-Iodo-1H-indazol-5-yl)amino)chromane-7-carbonitrile

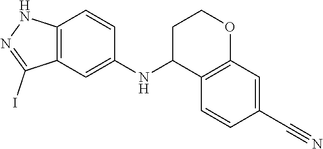 [SMILES: N#Cc1ccc2c(c1)OCCC2Nc1ccc2[nH]nc(I)c2c1]

To a stirred solution of 3-iodo-1H-indazol-5-amine (897.56 mg, 3.46 mmol) and 4-oxo-2,3-dihydrochromene-7-carbonitrile (500.0 mg, 2.89 mmol) in 1,4-dioxane (28.87 mL) was added 4-methylbenzenesulfonic acid; hydrate (54.92 mg, 0.290 mmol) and the mixture was stirred at 100° C. for 2.5 hrs. The reaction was left reach r.t. and then heated to 40° C., sodium triacetoxyborohydride (0.56 g, 2.89 mmol) was added portionwise over 3 hrs, during which the reaction mixture was left stirring at 40° C. The reaction mixture was diluted with H₂O and extracted with EtOAc. The organic phase was washed with saturated aqueous NaHCO₃ and brine, dried over anhydrous Na₂SO₄, filtered and evaporated to dryness. The residue was purified by reversed phase chromatography using a 2-100% CH₃CN/H₂O (0.1% HCOOH) gradient eluent to afford the title compound (156 mg, 13%). MS-ESI (m/z) calc'd for C₁₇H₁₄IN₄O [M+H]⁺: 417.0. Found 417.0.

### Step 2: 4-((3-(Isoxazol-4-yl)-1H-indazol-5-yl)amino)chromane-7-carbonitrile

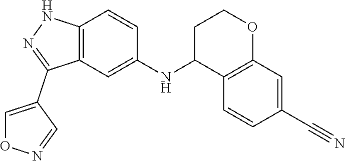 [SMILES: N#Cc1ccc2c(c1)OCCC2Nc1ccc2[nH]nc(-c3cnoc3)c2c1]

Prepared as described for 2-chloro-8-{[3-(1,3-oxazol-5-yl)-1H-indazol-5-yl]amino}-5,6,7,8-tetrahydroquinoline-3-carbonitrile using 4-((3-iodo-1H-indazol-5-yl)amino)chromane-7-carbonitrile in place of 2-chloro-8-[(3-iodo-1H-indazol-5-yl)amino]-5,6,7,8-tetrahydroquinoline-3-carbonitrile and isoxazol-4-ylboronic acid in place of oxazole-5-boronic acid pinacol ester to afford the title compound (21 mg, 42%).

### Step 3: 4-((3-(Isoxazol-4-yl)-1H-indazol-5-yl)amino)chromane-7-carbonitrile, enantiomer 1 and 2

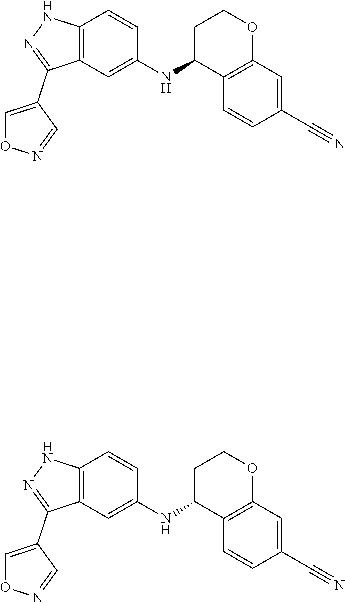 [SMILES: N#Cc1ccc2c(c1)OCC[C@@H]2Nc1ccc2[nH]nc(-c3cnoc3)c2c1 ; N#Cc1ccc2c(c1)OCC[C@H]2Nc1ccc2[nH]nc(-c3cnoc3)c2c1]

4-((3-(Isoxazol-4-yl)-1H-indazol-5-yl)amino)chromane-7-carbonitrile was purified by chiral separation using Method DA to afford 4-((3-(isoxazol-4-yl)-1H-indazol-5-yl)amino)chromane-7-carbonitrile, enantiomer 1 (7.2 mg, 14%) was obtained as a white solid. ¹H NMR (400 MHz, MeOD) δ 9.30 (s, 1H), 8.94 (s, 1H), 7.55 (d, J=7.9 Hz, 1H), 7.39 (dd, J=8.9, 0.8 Hz, 1H), 7.20 (dd, J=7.9, 1.6 Hz, 1H), 7.16 (d, J=1.6 Hz, 1H), 7.08 (d, J=2.0 Hz, 1H), 7.04 (dd, J=8.9, 2.1 Hz, 1H), 4.91 (t, J=5.2 Hz, 1H), 4.40-4.29 (m, 2H), 2.31-2.07 (m, 2H). MS-ESI (m/z) calc'd for C₂₀H₁₆N₅O₂ [M+H]⁺: 358.1. Found 358.1. A later eluting fraction was also isolated to afford 4-((3-(isoxazol-4-yl)-1H-indazol-5-yl)amino)chromane-7-carbonitrile, enantiomer 2 (6.8 mg, 14%) as a white solid. ¹H NMR (400 MHz, MeOD) δ 9.30 (s, 1H), 8.94 (s, 1H), 7.20 (dd, J=7.9, 1.7 Hz, 1H), 7.16 (d, J=1.6 Hz, 1H), 7.08 (d, J=2.0 Hz, 1H), 7.04 (dd, J=8.9, 2.1 Hz, 1H), 4.91 (t, J=5.2 Hz, 1H), 4.34 (dd, J=6.6, 4.2 Hz, 2H), 2.34-2.05 (m, 2H). MS-ESI (m/z) calc'd for C₂₀H₁₆N₅₀₂ [M+H]⁺: 358.1. Found 358.1.

### Example 193: 4-((3-Iodo-1H-indazol-5-yl)amino)chromane-7-carbonitrile, enantiomer 1 and 2

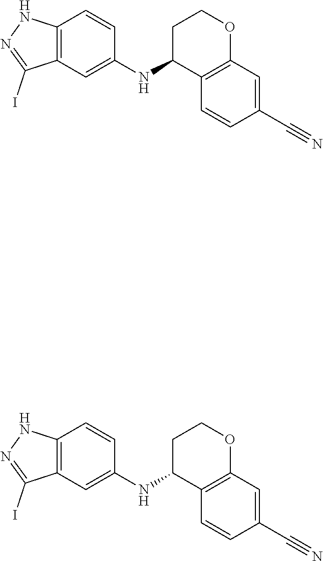 [SMILES: N#Cc1ccc2c(c1)OCC[C@@H]2Nc1ccc2[nH]nc(I)c2c1 ; N#Cc1ccc2c(c1)OCC[C@H]2Nc1ccc2[nH]nc(I)c2c1]

Prepared as described for 8-((3-bromo-1H-indazol-5-yl)amino)-5,6,7,8-tetrahydroquinoline-3-carbonitrile, using 3-iodo-1H-indazol-5-amine in place of 3-bromo-1H-indazol-5-amine and 4-oxo-3,4-dihydro-2H-1-benzopyran-7-carbonitrile in place of 8-oxo-5,6,7,8-tetrahydroquinoline-3-carbonitrile, to afford 4-((3-iodo-1H-indazol-5-yl)amino)-3,4-dihydro-2H-1-benzopyran-7-carbonitrile (35 mg, 10%). The mixture was purified by chiral separation using Method DP to afford 4-((3-iodo-1H-indazol-5-yl)amino)chromane-7-carbonitrile, enantiomer 1 (13.3 mg, 4%) as a white solid. ¹H NMR (400 MHz, DMSO-d₆) δ 13.11 (s, 1H), 7.48 (d, J=7.8 Hz, 1H), 7.39-7.21 (m, 3H), 7.00 (dd, J=8.9, 2.1 Hz, 1H), 6.50 (d, J=2.1 Hz, 1H), 6.09 (d, J=8.8 Hz, 1H), 4.95-4.71 (m, 1H), 4.44-4.16 (m, 2H), 2.18-2.06 (m, 1H), 2.06-1.94 (m, 1H). MS-ESI (m/z) calc'd for C₁₇H₁₄IN₄O [M+H]⁺: 417.0. Found 417.0. A later eluting fraction was also isolated to afford 4-((3-iodo-1H-indazol-5-yl)amino)chromane-7-carbonitrile, enantiomer 2 (13.9 mg, 4%) as a white solid. ¹H NMR (400 MHz, DMSO-d₆) δ 13.11 (s, 1H), 7.48 (d, J=7.8 Hz, 1H), 7.39-7.22 (m, 3H), 7.00 (dd, J=9.0, 2.1 Hz, 1H), 6.50 (d, J=2.1 Hz, 1H), 6.09 (d, J=8.8 Hz, 1H), 4.87-4.74 (m, 1H), 4.39-4.23 (m, 2H), 2.18-2.06 (m, 1H), 2.06-1.95 (m, 1H). MS-ESI (m/z) calc'd for C₁₇H₁₄IN₄O [M+H]⁺: 417.0. Found 417.1.

### Example 194: 4-((3-(Oxazol-5-yl)-1H-indazol-5-yl)amino)-3,4-dihydro-2H-pyrano[3,2-b]pyridine-7-carbonitrile, enantiomer 1 and 2

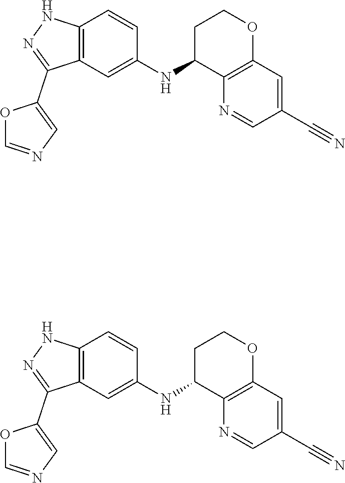 [SMILES: N#Cc1cnc2c(c1)OCC[C@@H]2Nc1ccc2[nH]nc(-c3cnco3)c2c1 ; N#Cc1cnc2c(c1)OCC[C@H]2Nc1ccc2[nH]nc(-c3cnco3)c2c1]

### Step 1: 5-Bromo-2-iodopyridin-3-ol

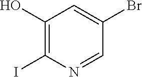

A mixture of 5-bromopyridin-3-ol (10 g, 57.47 mmol), 12 (14.59 g, 57.47 mmol), and Na₂CO₃ (12.79 g, 120.69 mmol) in H₂O (200 mL) was degassed and purged with N₂ (3×) and then the mixture was stirred at 25° C. for 3 hrs under an N₂ atmosphere. The reaction mixture was acidified with 1 M HCl to pH=5 and extracted with EtOAc (3×). The combined organic layers were dried over Na₂SO₄, filtered, and concentrated to afford the title compound (13 g, 75%) as a brown solid. ¹H NMR (400 MHz, DMSO-d₆) δ 11.40 (s, 1H), 8.01 (d, J=2.13 Hz, 1H), 7.30 (d, J=2.13 Hz, 1H). MS-ESI (m/z) calc'd for C₅H₄BrINO [M+H]⁺: 299.8, 301.8. Found 299.8, 301.8.

### Step 2: 5-Bromo-3-(but-3-en-1-yloxy)-2-iodopyridine

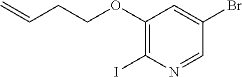

To a solution of 5-bromo-2-iodopyridin-3-ol (5 g, 16.67 mmol) in THF (20 mL) was added but-3-en-1-ol (1.20 g, 16.67 mmol), PPh₃ (5.25 g, 20.01 mmol) and DIAD (3.71 g, 18.34 mmol) at 0° C. The mixture was stirred at 70° C. for 1 hr. The reaction mixture was combined with another 1 g scale reaction before work up. The final mixture was concentrated to give a residue. The residue was diluted with H₂O and extracted with EtOAc (3×). The combined organic phases were dried over anhydrous Na₂SO₄, filtered, and the filtrate was concentrated to give a residue. The residue was purified by flash silica gel column chromatography (ISCO; 40 g SepaFlash column) using a 0-3% EtOAc/petroleum ether gradient eluent to afford the title compound (4.79 g, 67%) as a colorless oil. ¹H NMR (400 MHz, DMSO-d₆) δ 8.12 (d, J=2.13 Hz, 1H), 7.63 (d, J=2.00 Hz, 1H), 5.92 (ddt, J=17.13, 10.38, 6.63, 6.63 Hz, 1H), 5.21 (dq, J=17.26, 1.67 Hz, 1H), 5.03-5.14 (m, 1H), 4.19 (t, J=6.38 Hz, 2H), 2.51-2.53 (m, 2H). MS-ESI (m/z) calc'd for C₉H₁₀BrINO [M+H]⁺: 353.9, 355.9. Found 354.0, 356.0.

### Step 3: 7-Bromo-4-methylene-3,4-dihydro-2H-pyrano[3,2-b]pyridine

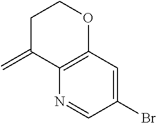

A mixture of 5-bromo-3-(but-3-en-1-yloxy)-2-iodopyridine (5.2 g, 14.69 mmol), Et₄NCl (4.87 g, 29.38 mmol), PPh₃ (1.16 g, 4.41 mmol), KOAc (720.86 mg, 7.35 mmol) and Pd(OAc)₂, (329.80 mg, 1.47 mmol) in DMF (50 mL) was degassed and purged with N₂ (3×) at 20° C. The mixture was then stirred at 120° C. for 12 hrs under an N₂ atmosphere. The reaction mixture was diluted with H₂O and extracted with EtOAc (3×). The combined organic phases were dried over anhydrous Na₂SO₄, filtered, and the filtrate was concentrated to give a residue. The residue was purified by flash silica gel column chromatography (ISCO; 20 g SepaFlash column) using a 0-7% EtOAc/petroleum ether gradient eluent to afford the title compound (1.94 g, 58%) as a yellow oil. ¹H NMR (400 MHz, CDCl₃) δ 8.16 (d, J=1.75 Hz, 1H), 7.22-7.29 (m, 1H), 6.11 (d, J=1.13 Hz, 1H), 5.02 (d, J=1.50 Hz, 1H), 4.08-4.23 (m, 2H), 2.73 (br t, J=5.69 Hz, 2H). MS-ESI (m/z) calc'd for C₉H₉BrNO [M+H]⁺: 226.0, 228.0. Found 226.1, 228.1.

### Step 4: 7-Bromo-2,3-dihydro-4H-pyrano[3,2-b]pyridin-4-one

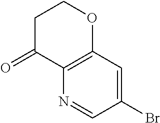 [SMILES: O=C1CCOc2cc(Br)cnc21]

Ozone was bubbled through a mixture of 7-bromo-4-methylene-3,4-dihydro-2H-pyrano[3,2-b]pyridine (500 mg, 2.21 mmol) in CH₂Cl₂ (25 mL) for 0.5 hr at −78° C. (15 psi). The reaction mixture turned blue. PPh₃ (2.90 g, 11.06 mmol) was then added to the mixture and the mixture was stirred at 20° C. for 12 hrs. The mixture was concentrated and purified by flash silica gel column chromatography (ISCO; 12 g SepaFlash column) using a 0-37% EtOAc/petroleum ether gradient eluent to afford the title compound (460 mg, 910%) as a pale yellow solid. ¹H NMR (400 MHz, DMSO-d₆) δ 8.47 (d, J=1.88 Hz, 1H), 7.96 (d, J=1.88 Hz, 1H), 4.65 (t, J=6.50 Hz, 2H), 2.92 (t, J=6.44 Hz, 2H). MS-ESI (m/z) calc'd for C₈H₇BrNO₂ [M+H]⁺: 228.0, 230.0. Found 227.9, 229.9.

### Step 5: 7-Bromo-N-(3-(oxazol-5-yl)-1H-indazol-5-yl)-3,4-dihydro-2H-pyrano[3,2-b]pyridin-4-amine

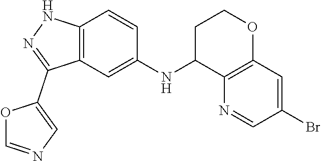 [SMILES: Brc1cnc2c(c1)OCCC2Nc1ccc2[nH]nc(-c3cnco3)c2c1]

To a solution of 3-(oxazol-5-yl)-1H-indazol-5-amine (368.71 mg, 1.84 mmol) in MeOH (10 mL) was added 7-bromo-2,3-dihydro-4H-pyrano[3,2-b]pyridin-4-one (420 mg, 1.84 mmol), AcOH (110.60 mg, 1.84 mmol) at 25° C. The mixture was stirred at 25° C. for 0.5 hr. Then NaBH₃CN (347.22 mg, 5.53 mmol) was added to the mixture at 25° C. The mixture was stirred at 25° C. for 12 hrs and then concentrated to give a residue. The residue was diluted with EtOAc and a brown solid formed. The mixture was filtered and the solid was washed with EtOAc (2×) and dried under vacuum to afford the title compound (250.8 mg, 33%) as a brown solid. MS-ESI (m/z) calc'd for C₁₈H₁₅BrN₅O₂ [M+H]⁺: 412.0, 414.0. Found 412.2, 414.1.

### Step 6: 4-((3-(Oxazol-5-yl)-1H-indazol-5-yl)amino)-3,4-dihydro-2H-pyrano[3,2-b]pyridine-7-carbonitrile

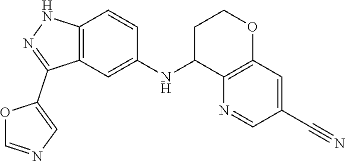 [SMILES: N#Cc1cnc2c(c1)OCCC2Nc1ccc2[nH]nc(-c3cnco3)c2c1]

A mixture of Zn(CN)₂ (170.91 mg, 1.46 mmol), 7-bromo-N-(3-(oxazol-5-yl)-1H-indazol-5-yl)-3,4-dihydro-2H-pyrano[3,2-b]pyridin-4-amine (200 mg, 485.15 umol), Zn (63.45 mg, 970.31 umol), DPPF (26.90 mg, 48.52 umol) and Pd₂(dba)₃ (44.43 mg, 48.52 umol) in DMA (5 mL) was degassed and purged with N₂ (3×) at 20° C. The mixture was then stirred at 100° C. for 2 hrs under an N₂ atmosphere. The reaction mixture was filtered and the filtrate was concentrated to give a residue. The residue was purified by preparative-HPLC using Method EP to afford the title compound (48 mg, 27%) as a green solid. MS-ESI (m/z) calc'd for C₁₉H₁₅N₆O₂ [M+H]⁺: 359.1. Found 359.3.

### Step 7: 4-((3-(Oxazol-5-yl)-1H-indazol-5-yl)amino)-3,4-dihydro-2H-pyrano[3,2-b]pyridine-7-carbonitrile, enantiomer 1 and 2

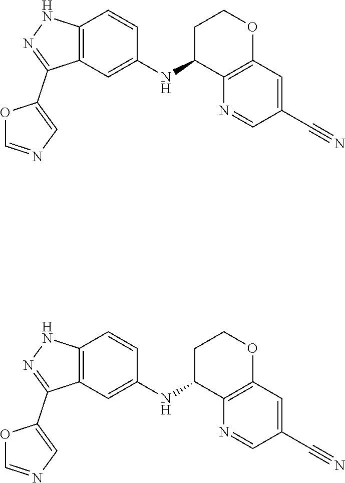 [SMILES: N#Cc1cnc2c(c1)OCC[C@@H]2Nc1ccc2[nH]nc(-c3cnco3)c2c1 ; N#Cc1cnc2c(c1)OCC[C@H]2Nc1ccc2[nH]nc(-c3cnco3)c2c1]

4-((3-(Oxazol-5-yl)-1H-indazol-5-yl)amino)-3,4-dihydro-2H-pyrano[3,2-b]pyridine-7-carbonitrile (9 mg) was subjected to chiral separation using Method EQ to afford 4-((3-(oxazol-5-yl)-1H-indazol-5-yl)amino)-3,4-dihydro-2H-pyrano[3,2-b]pyridine-7-carbonitrile, enantiomer 1 (3.29 mg, 36%) as a yellow solid. ¹H NMR (400 MHz, DMSO-d₆) δ 13.12 (s, 1H), 8.56 (d, J=1.83 Hz, 1H), 8.48 (s, 1H), 7.88 (d, J=1.83 Hz, 1H), 7.71 (s, 1H), 7.39 (d, J=8.93 Hz, 1H), 7.15 (s, 1H), 7.00 (dd, J=8.99, 2.02 Hz, 1H), 6.15 (d, J=7.46 Hz, 1H), 4.84-4.94 (m, 1H), 4.29-4.48 (m, 2H), 2.12-2.32 (m, 2H). MS-ESI (m/z) calc'd for C₁₉H₁₅N₆O₂ [M+H]⁺: 359.1. Found 359.0. A later eluting fraction was also isolated to afford 4-((3-(oxazol-5-yl)-1H-indazol-5-yl)amino)-3,4-dihydro-2H-pyrano[3,2-b]pyridine-7-carbonitrile, enantiomer 2 (3.07 mg, 34%) as a yellow solid. ¹H NMR (400 MHz, DMSO-d₆) δ 13.13 (br s, 1H), 8.56 (d, J=1.83 Hz, 1H), 8.48 (s, 1H), 7.88 (d, J=1.83 Hz, 1H), 7.71 (s, 1H), 7.39 (d, J=9.05 Hz, 1H), 7.14 (s, 1H), 7.00 (dd, J=9.05, 2.08 Hz, 1H), 6.15 (d, J=7.46 Hz, 1H), 4.85-4.93 (m, 1H), 4.30-4.47 (m, 2H), 2.12-2.31 (m, 2H). MS-ESI (m/z) calc'd for C₁₉H₁₅N₆O₂ [M+H]⁺: 359.1. Found 359.0.

### Example A. LRRK2 Kinase Activity

LRRK2 kinase activity was measured using a LanthaScreen™ Kinase Activity Assay from ThermoFisher Scientific. Recombinant wild type or G2019S-LRRK2 protein (Life Technologies, PR8604B or PV4881, respectively), was incubated with a fluorescein-labeled peptide substrate called LRRKtide that is based upon ezrin/radixin/moesin (ERM) (Life Technologies, PV4901) in the presence of ATP and serially diluted compound. After an incubation period of 1 hr, the phosphotransferase activity was stopped and a terbium-labelled anti-pERM antibody (Life Technologies, PV4899) was added to detect the phosphorylation of LRRKtide by measuring the time resolved-Förster resonant energy transfer (TR-FRET) signal from the terbium label on the antibody to the fluorescein tag on LRRKtide, expressed as the 520 nm/495 nm emission ratio. Compound-dependent inhibition of the TR-FRET signal was used to generate a concentration-response curve for IC₅₀ determination.

The assay was carried out under the following protocol conditions: 1 mM compound in DMSO was serially diluted 1:3, 11 points in DMSO with a Biomek FX and 0.1 μL of the diluted compound was subsequently stamped into the assay plate (384-well format Lumitrac 200, Greiner, 781075) with an Echo Labcyte such that the final compound concentration in the assay was 10 μM to 169 μM. Subsequently, 5 μL of 2×kinase solution (2.9 nM final concentration) was added to the assay plate in assay buffer composed of 50 mM Tris pH 8.5 (Sigma, T6791), 5 mM MgCl₂ (Fluka, 63020), 1 mM EGTA (Sigma, E3889), 0.01% BRIJ-35 (Sigma, P1254) and 2 mM DTT. The reaction was started by addition of 2×ATP/LRRKtide solution in assay buffer such that the final concentration was 400 nM LRRKtide and 25 μM ATP. After 60 min incubation at room temperature, the reaction was stopped by addition of 10 μL of 2×stop solution containing a final concentration of 2 nM anti-pERM antibody and 10 mM EDTA. After a 30 min incubation at RT, the TR-FRET signal was measured on a Wallac 2104 EnVision® multilabel reader at an excitation wavelength of 340 nm and reading emission at 520 nm and 495 nm. The ratio of the 520 nm and 495 nm emission was used to analyze the data.

The Results of the LRRK2 kinase activity assay are shown in Table A-1. Data is displayed as follows: + is IC₅₀≤100 nM; ++ is 100 nM<IC₅₀≤1,000 nM; and +++ is 1,000 nM<IC₅₀≤10,000 nM.

TABLE A-1
LRRK2 Kinase Activity Assay

| | LRRK2 WT | LRRK2 G2019S |
|---|---|---|
| Example No. | IC₅₀ (nM) | IC₅₀ (nM) |
| 1, enantiomer 1 | ++ | + |
| 1, enantiomer 2 | ++ | + |
| 2, enantiomer 1 | + | + |
| 2, enantiomer 2 | + | + |
| 3, enantiomer 1 | + | + |
| 3, enantiomer 2 | + | + |
| 4, enantiomer 1 | ++ | + |
| 4, enantiomer 2 | + | + |
| 5, enantiomer 1 | + | + |
| 5, enantiomer 2 | + | + |
| 6, enantiomer 1 | + | + |
| 6, enantiomer 2 | ++ | + |
| 7, enantiomer 1 | +++ | ++ |
| 7, enantiomer 2 | + | + |
| 8, enantiomer 1 | + | + |
| 8, enantiomer 2 | + | + |
| 9, enantiomer 1 | + | + |
| 9, enantiomer 2 | + | + |
| 10 | ++ | + |
| 11, enantiomer 1 | ++ | + |
| 11, enantiomer 2 | + | + |
| 12, enantiomer 1 | + | + |
| 12, enantiomer 2 | + | + |
| 13 | ++ | + |
| 14, enantiomer 1 | + | + |
| 14, enantiomer 2 | + | + |
| 15, enantiomer 1 | ++ | + |
| 15, enantiomer 2 | +++ | ++ |
| 16, enantiomer 1 | + | + |
| 16, enantiomer 2 | + | + |
| 17, enantiomer 1 | + | + |
| 17, enantiomer 2 | ++ | + |
| 18, enantiomer 1 | + | + |
| 18, enantiomer 2 | + | + |
| 19, enantiomer 1 | ++ | ++ |
| 19, enantiomer 2 | ++ | + |
| 20, enantiomer 1 | + | + |
| 20, enantiomer 2 | ++ | + |
| 21, enantiomer 1 | + | + |
| 21, enantiomer 2 | ++ | + |
| 22, enantiomer 1 | + | + |
| 22, enantiomer 2 | + | + |
| 23, enantiomer 1 | + | + |
| 23, enantiomer 2 | + | + |
| 24, enantiomer 1 | + | + |
| 24, enantiomer 2 | + | + |
| 25, enantiomer 1 | + | + |
| 25, enantiomer 2 | + | + |
| 26, enantiomer 1 | + | + |
| 26, enantiomer 2 | + | + |
| 27, enantiomer 1 | + | + |
| 27, enantiomer 2 | + | + |
| 28, enantiomer 1 | ++ | + |
| 28, enantiomer 2 | + | + |
| 29, enantiomer 1 | + | + |
| 29, enantiomer 2 | + | + |
| 30, enantiomer 1 | + | + |
| 30, enantiomer 2 | ++ | + |
| 31, enantiomer 1 | + | + |
| 31, enantiomer 2 | ++ | + |
| 32, enantiomer 1 | +++ | ++ |
| 32, enantiomer 2 | + | + |
| 33, enantiomer 1 | + | + |
| 33, enantiomer 2 | ++ | ++ |
| 34 | ++ | + |
| 35, enantiomer 1 | + | + |
| 35, enantiomer 2 | ++ | ++ |
| 36, enantiomer 1 | +++ | ++ |
| 36, enantiomer 2 | + | + |
| 37, enantiomer 1 | + | + |
| 37, enantiomer 2 | + | + |
| 38, enantiomer 1 | + | + |
| 38, enantiomer 2 | ++ | + |
| 39, enantiomer 1 | + | + |
| 39, enantiomer 2 | + | + |
| 40, enantiomer 1 | ++ | + |
| 40, enantiomer 2 | + | + |
| 41, enantiomer 1 | + | + |
| 41, enantiomer 2 | + | + |
| 42, enantiomer 1 | + | + |
| 42, enantiomer 2 | + | + |
| 43, enantiomer 1 | ++ | + |
| 43, enantiomer 2 | +++ | ++ |
| 44 | + | + |
| 45 | >10,000 | +++ |
| 46 | >10,000 | ++ |
| 47, enantiomer 1 | ++ | ++ |
| 47, enantiomer 2 | + | + |
| 48, enantiomer 1 | + | + |
| 48, enantiomer 2 | + | + |
| 49, enantiomer 1 | + | + |
| 49, enantiomer 2 | ++ | + |
| 50, enantiomer 1 | + | + |
| 50, enantiomer 2 | + | ++ |
| 51, enantiomer 1 | + | + |
| 51, enantiomer 2 | + | + |
| 52, enantiomer 1 | + | + |
| 52, enantiomer 2 | + | + |
| 53, enantiomer 1 | + | + |
| 53, enantiomer 2 | ++ | + |
| 54 | +++ | ++ |
| 55 | + | + |
| 56 | +++ | ++ |
| 57 | + | + |
| 58 | ++ | + |
| 59 | + | + |
| 60 | + | + |
| 61 | ++ | + |
| 62 | + | + |
| 63 | ++ | + |
| 64 | + | + |
| 65 | +++ | ++ |
| 66 | ++ | + |
| 67 | + | + |
| 68 | + | + |
| 69 | + | + |
| 70 | ++ | + |
| 71 | + | + |
| 72 | + | + |
| 73 | ++ | + |
| 74 | + | + |
| 75 | ++ | + |
| 76 | + | + |
| 77 | + | + |
| 78 | ++ | + |
| 79 | ++ | + |
| 80 | >10,000 | ++ |
| 81 | + | + |
| 82 | + | + |
| 83 | + | + |
| 84 | ++ | + |
| 85 | + | + |
| 86 | + | + |
| 87 | +++ | ++ |
| 88 | ++ | + |
| 89 | + | + |
| 90 | + | + |
| 91 | ++ | + |
| 92 | ++ | + |
| 93 | + | + |
| 94 | >10,000 | +++ |
| 95 | +++ | + |
| 96 | + | + |
| 97 | ++ | + |
| 98 | + | + |
| 99 | ++ | + |
| 100 | + | + |
| 101, enantiomer 1 | + | + |
| 101, enantiomer 2 | + | + |
| 102, enantiomer 1 | + | + |
| 102, enantiomer 2 | +++ | ++ |
| 103, enantiomer 1 | + | + |
| 103, enantiomer 2 | + | + |
| 104, enantiomer 1 | + | + |
| 104, enantiomer 2 | ++ | ++ |
| 105, enantiomer 1 | ++ | + |
| 105, enantiomer 2 | ++ | + |
| 106, enantiomer 1 | ++ | + |
| 106, enantiomer 2 | + | + |
| 107, enantiomer 1 | + | + |
| 107, enantiomer 2 | ++ | + |
| 108, enantiomer 1 | ++ | + |
| 108, enantiomer 2 | + | + |
| 109, enantiomer 1 | + | + |
| 109, enantiomer 2 | ++ | + |
| 110, enantiomer 1 | + | + |
| 110, enantiomer 2 | ++ | + |
| 111, enantiomer 1 | + | + |
| 111, enantiomer 2 | ++ | + |
| 112, enantiomer 1 | ++ | + |
| 112, enantiomer 2 | + | + |
| 113, enantiomer 1 | + | + |
| 113, enantiomer 2 | + | + |
| 114, enantiomer 1 | ++ | ++ |
| 114, enantiomer 2 | + | + |
| 115, enantiomer 1 | ++ | + |
| 115, enantiomer 2 | ++ | + |
| 116, enantiomer 1 | + | + |
| 116, enantiomer 2 | + | + |
| 117, enantiomer 1 | + | + |
| 117, enantiomer 2 | ++ | ++ |
| 118, enantiomer 1 | + | + |
| 118, enantiomer 2 | + | + |
| 119, enantiomer 1 | ++ | + |
| 119, enantiomer 2 | + | + |
| 120, enantiomer 1 | + | + |
| 120, enantiomer 2 | + | + |
| 121, enantiomer 1 | + | + |
| 121, enantiomer 2 | + | + |
| 122, enantiomer 1 | ++ | + |
| 122, enantiomer 2 | + | + |
| 123, enantiomer 1 | + | + |
| 123, enantiomer 2 | + | + |
| 124, enantiomer 1 | ++ | + |
| 124, enantiomer 2 | ++ | ++ |
| 125, enantiomer 1 | ++ | + |
| 125, enantiomer 2 | +++ | ++ |
| 126, enantiomer 1 | +++ | +++ |
| 126, enantiomer 2 | ++ | + |
| 127, enantiomer 1 | ++ | ++ |
| 127, enantiomer 2 | ++ | ++ |
| 128, enantiomer 1 | ++ | ++ |
| 128, enantiomer 2 | ++ | + |
| 129, enantiomer 1 | +++ | ++ |
| 129, enantiomer 2 | ++ | ++ |
| 130, enantiomer 1 | +++ | ++ |
| 130, enantiomer 2 | ++ | + |
| 131, enantiomer 1 | + | + |
| 131, enantiomer 2 | + | + |
| 132, enantiomer 1 | + | + |
| 132, enantiomer 2 | + | + |
| 133, enantiomer 1 | +++ | ++ |
| 133, enantiomer 2 | +++ | + |
| 134, enantiomer 1 | +++ | ++ |
| 134, enantiomer 2 | +++ | ++ |
| 135, enantiomer 1 | ++ | + |
| 135, enantiomer 2 | ++ | + |
| 136, enantiomer 1 | ++ | + |
| 136, enantiomer 2 | ++ | ++ |
| 137, enantiomer 1 | + | + |
| 137, enantiomer 2 | ++ | + |
| 138, enantiomer 1 | + | + |
| 138, enantiomer 2 | ++ | + |
| 139, enantiomer 1 | +++ | +++ |
| 139, enantiomer 2 | +++ | + |
| 140, enantiomer 1 | ++ | + |
| 140, enantiomer 2 | ++ | + |
| 141, enantiomer 1 | + | + |
| 141, enantiomer 2 | + | + |
| 142, enantiomer 1 | ++ | + |
| 142, enantiomer 2 | + | + |
| 143, enantiomer 1 | ++ | + |
| 143, enantiomer 2 | + | + |
| 144, enantiomer 1 | ++ | ++ |
| 144, enantiomer 2 | + | + |
| 145, enantiomer 1 | ++ | + |
| 145, enantiomer 2 | + | + |
| 146, enantiomer 1 | ++ | + |
| 146, enantiomer 2 | + | + |
| 147, enantiomer 1 | + | + |
| 147, enantiomer 2 | ++ | + |
| 148, enantiomer 1 | + | + |
| 148, enantiomer 2 | ++ | ++ |
| 149, enantiomer 1 | ++ | ++ |
| 149, enantiomer 2 | + | + |
| 150, enantiomer 1 | ++ | ++ |
| 150, enantiomer 2 | + | + |
| 151, enantiomer 1 | ++ | + |
| 151, enantiomer 2 | + | + |
| 152, enantiomer 1 | +++ | +++ |
| 152, enantiomer 2 | +++ | + |
| 153, enantiomer 1 | +++ | + |
| 153, enantiomer 2 | +++ | +++ |
| 154, enantiomer 1 | ++ | |
| 154, enantiomer 2 | + | + |
| 155, enantiomer 1 | + | + |
| 155, enantiomer 2 | + | + |
| 156, enantiomer 1 | + | + |
| 156, enantiomer 2 | + | + |
| 157, enantiomer 1 | ++ | ++ |
| 157, enantiomer 2 | + | + |
| 158, enantiomer 1 | + | + |
| 158, enantiomer 2 | + | + |
| 159, enantiomer 1 | + | + |
| 159, enantiomer 2 | + | + |
| 160, enantiomer 1 | + | + |
| 160, enantiomer 2 | + | + |
| 161, enantiomer 1 | + | + |
| 161, enantiomer 2 | ++ | + |
| 162, enantiomer 1 | ++ | ++ |
| 162, enantiomer 2 | + | + |
| 163, enantiomer 1 | ++ | + |
| 163, enantiomer 2 | + | + |
| 164, enantiomer 1 | ++ | + |
| 164, enantiomer 2 | + | + |
| 165, enantiomer 1 | ++ | + |
| 165, enantiomer 2 | + | + |
| 166, enantiomer 1 | + | + |
| 166, enantiomer 2 | + | + |
| 167, enantiomer 1 | ++ | + |
| 167, enantiomer 2 | + | + |
| 168, enantiomer 1 | ++ | + |
| 168, enantiomer 2 | ++ | + |
| 169, enantiomer 1 | ++ | + |
| 169, enantiomer 2 | ++ | + |
| 170, enantiomer 1 | + | + |
| 170, enantiomer 2 | ++ | + |
| 171, enantiomer 1 | + | + |
| 171, enantiomer 2 | + | + |
| 172, enantiomer 1 | ++ | ++ |
| 172, enantiomer 2 | + | + |
| 173, enantiomer 1 | + | + |
| 173, enantiomer 2 | ++ | + |
| 174, enantiomer 1 | + | + |
| 174, enantiomer 2 | ++ | + |
| 175, enantiomer 1 | ++ | + |
| 175, enantiomer 2 | ++ | + |
| 176, enantiomer 1 | ++ | + |
| 176, enantiomer 2 | + | + |
| 177, enantiomer 1 | + | + |
| 177, enantiomer 2 | + | + |
| 178, enantiomer 1 | + | + |
| 178, enantiomer 2 | + | + |
| 179, enantiomer 1 | + | + |
| 179, enantiomer 2 | ++ | ++ |
| 180, enantiomer 1 | + | + |
| 180, enantiomer 2 | + | + |
| 181, enantiomer 1 | + | + |
| 181, enantiomer 2 | ++ | + |
| 182, enantiomer 1 | + | + |
| 182, enantiomer 2 | + | + |
| 183, enantiomer 1 | +++ | ++ |
| 183, enantiomer 2 | + | + |
| 184, enantiomer 1 | + | + |
| 184, enantiomer 2 | +++ | + |
| 185, enantiomer 1 | + | + |
| 185, enantiomer 2 | +++ | ++ |
| 186, enantiomer 1 | + | + |
| 186, enantiomer 2 | +++ | ++ |
| 187, enantiomer 1 | + | + |
| 187, enantiomer 2 | ++ | ++ |
| 188, enantiomer 1 | + | + |
| 188, enantiomer 2 | +++ | ++ |
| 189, enantiomer 1 | +++ | ++ |
| 189, enantiomer 2 | ++ | + |
| 190, enantiomer 1 | + | + |
| 190, enantiomer 2 | + | + |
| 191, enantiomer 1 | ++ | + |
| 191, enantiomer 2 | + | + |
| 192, enantiomer 1 | ++ | ++ |
| 192, enantiomer 2 | + | + |
| 193, enantiomer 1 | +++ | ++ |
| 193, enantiomer 2 | + | + |
| 194, enantiomer 1 | + | + |
| 194, enantiomer 2 | ++ | ++ |

Various modifications of the invention, in addition to those described herein, will be apparent to those skilled in the art from the foregoing description. Such modifications are also intended to fall within the scope of the appended claims. Each reference, including all patent, patent applications, and publications, cited in the present application is incorporated herein by reference in its entirety.

What is claimed is:

1. A compound of Formula I: 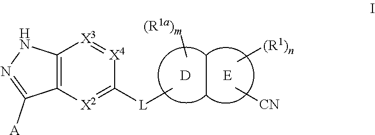
or a pharmaceutically acceptable salt thereof, wherein:
A is selected from Cy¹, Cy¹-C₁₋₄ alkyl-, Cy¹-C₂₋₄ alkenyl-, halo, C₁₋₆ alkyl, C₂₋₆ alkenyl, C₂₋₆ alkynyl, C₁₋₆ haloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, C(═NR^{e})NR^{c}R^{d}, NR^{c}C(═NR^{e})NR^{c}R^{d}, NR^{c}R^{d}, NR^{c}C(O)R^{b}, NR^{c}C(O)OR^{a}, NR^{c}C(O)NR^{c}R^{d}, NR^{c}C(S)NR^{c}R^{d}, NR^{c}S(O)R^{b}, NR^{c}S(O)₂R^{b}, NR^{c}S(O)₂NR^{c}R^{d}, S(O)R^{b}, S(O)NR^{e}R^{d}, S(O)₂R^{b}, and S(O)₂NR^{e}R^{d}; wherein said C₁₋₆ alkyl, C₂₋₆ alkenyl, C₂₋₆ alkynyl, and C₁₋₆ haloalkyl of A are each optionally substituted with 1, 2, 3, 4, or 5 substituents independently selected from halo, C₁₋₆ alkyl, C₂₋₆ alkenyl, C₂₋₆ alkynyl, C₁₋₆ haloalkyl, C₃₋₇ cycloalkyl, CN, NO₂, OR^{a}, SR^{a}, C(O)R^{b}, C(O)NR^{e}R^{d}, C(O)OR^{a}, OC(O)R^{b}, OC(O)NR^{c}R^{d}, C(═NR^{e})NR^{c}R^{d}, NR^{c}C(═NR^{e})NR^{c}R^{d}, NR^{c}R^{d}, NR^{c}C(O)R^{b}, NR^{c}C(O)OR^{a}, NR^{c}C(O)NR^{c}R^{d}, NR^{c}S(O)R^{b}, NR^{c}S(O)₂R^{b}, NR^{c}S(O)₂NR^{c}R^{d}, S(O)R^{b}, S(O)NR^{c}R^{d}, S(O)₂R^{b}, and S(O)₂NR^{c}R^{d};
L is O, S, or NR^{N};
R^{N} is H or C₁₋₄ alkyl;
X² is N or CR²;
X³ is N or CR³;
X⁴ is N or CR⁴; wherein not more than two of X², X³, and X⁴ are simultaneously N;
Ring D is a C₄₋₇ cycloalkyl group or a 4-7 membered heterocycloalkyl group, each of which is fused with Ring E;
Ring E is phenyl or a 5- to 6-membered heteroaryl group, fused with Ring D;
Cy¹ is selected from C₆₋₁₀ aryl, C₃₋₁₀ cycloalkyl, 5-14 membered heteroaryl, and 4-14 membered heterocycloalkyl, each optionally substituted by 1, 2, 3, 4, or 5 substituents independently selected from halo, C₁₋₆ alkyl, C₂₋₆ alkenyl, C₂₋₆ alkynyl, C₁₋₆ haloalkyl, C₆₋₁₀ aryl, C₃₋₁₀ cycloalkyl, 5-14 membered heteroaryl, 4-14 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, C(O)N(R^{c})OR^{a}, NR^{c}R^{d}, NR^{c}C(O)R^{b}, NR^{c}C(O)OR^{a}, NR^{c}C(O)NR^{c}R^{d}, C(═NR^{e})R^{b}, C(═NR^{e})NR^{c}R^{d}, NR^{c}C(═NR^{e})NR^{c}R^{d}, NR^{c}S(O)R^{b}, NR^{c}S(O)₂R^{b}, NR^{c}S(O)₂NR^{c}R^{d}, S(O)R^{b}, S(O)NR^{c}R^{d}, S(O)₂R^{b}, and S(O)₂NR^{c}R^{d}, wherein said substituents C₁₋₆ alkyl, C₂₋₆ alkenyl, C₂₋₆ alkynyl, C₁₋₆ haloalkyl, C₆₋₁₀ aryl, C₃₋₁₀ cycloalkyl, 5-14 membered heteroaryl, 4-14 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl are each optionally substituted by 1, 2, or 3 further substituents independently selected from halo, C₁₋₆ alkyl, C₂₋₆ alkenyl, C₂₋₆ alkynyl, C₁₋₆ haloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)OR^{a}, NR^{c}C(O)NR^{c}R^{d}, C(═NR^{e})R^{b}, C(═NR^{e})NR^{c}R^{d}, NR^{c}C(═NR^{e})NR^{c}R^{d}, NR^{c}S(O)R^{b}, NR^{c}S(O)₂R^{b}, NR^{c}S(O)₂NR^{c}R^{d}, S(O)R^{b}, S(O)NR^{c}R^{d}, S(O)₂R^{b}, and S(O)₂NR^{c}R^{d};
each R¹ and R^{1a} is independently selected from H, D, halo, C₁₋₆ alkyl, C₂₋₆ alkenyl, C₂-6 alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}C(S)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}; wherein said C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R¹ are each optionally substituted with 1, 2, 3, 4, or 5 substituents independently selected from halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};
or two R¹ groups together with the atoms to which they are attached form a C₃₋₇ cycloalkyl group which is optionally substituted with 1, 2, 3, 4, or 5 substituents independently selected from halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{a1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{a1}, S(O)₂R^{b1}, and S(O)₂NR^{c1}R^{d1};
or two R^{1a} groups together with the atoms to which they are attached form a C₃₋₇ cycloalkyl group which is optionally substituted with 1, 2, 3, 4, or 5 substituents independently selected from halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};
R² and R⁴ are each independently selected from H, D, halo, C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a}2, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2}, wherein said C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R² and R⁴ are each optionally substituted with 1, 2, 3, 4, or 5 substituents independently selected from halo, C₁₋₆ alkyl, C₂₋₆ alkenyl, C₂₋₆ alkynyl, C₁₋₆ haloalkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2};
R³ is selected from H, D, halo, C₁₋₆ alkyl, C₂₋₆ alkenyl, C₂₋₆ alkynyl, C₁₋₆ haloalkyl, C₃-4 cycloalkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, C(═NR^{e3})R^{b3}, C(═NR^{e3})NR^{c3}R^{d3}, NR^{c3}C(═NR^{e3})NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3}, wherein said C₁₋₆ alkyl, C₂₋₆ alkenyl, C₂₋₆ alkynyl, C₁₋₆ haloalkyl, and C₃₋₄ cycloalkyl of R³ are each optionally substituted with 1, 2, or 3 substituents independently selected from halo, C₁₋₄ alkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, C(═NR^{e3})R^{b3}, C(═NR^{e3})NR^{c3}R^{d3}, NR^{c3}C(═NR^{e3})NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3};
each R^{a}, R^{b}, R^{c}, R^{d}, R^{a1}, R^{b1}, R^{c1}, R^{d1}, R^{a2}, R^{b2}, R^{c2}, and R^{d2} is independently selected from H, D, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl, wherein said C₁₋₆ alkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{a}, R^{b}, R^{c}, R^{d}, R^{a1}, R^{b1}, R^{c1}, R^{d1}, R^{a2}, R^{b2}, R^{c2}, and R^{d2} is optionally substituted with 1, 2, 3, 4, or 5 substituents independently selected from halo, C₁₋₄ alkyl, C₁₋₄ haloalkyl, C₁₋₆ haloalkyl, C₂₋₆ alkenyl, C₂₋₆ alkynyl, CN, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)NR^{c3}R^{d3}, NR^{c3}C(O)OR^{a3}, C(═NR^{e3})NR^{c3}R^{d3}, NR^{c3}C(═NR^{e3})NR^{c3}R^{d3}, S(O)R^{b3}, S(O)NR^{c3}R^{d3}, S(O)₂R^{b3}, NR^{c3}S(O)₂R^{b3}, NR^{c3}S(O)₂NR^{c3}R^{d3}, and S(O)₂NR^{c3}R^{d3};
each R^{a3}, R^{b3}, R^{c3}, and R^{d3} is independently selected from H, D, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-6 membered heteroaryl, and 4-7 membered heterocycloalkyl, wherein said C₁₋₆ alkyl, C₁₋₆ haloalkyl, C₂₋₆ alkenyl, C₂₋₆ alkynyl, C₆₋₁₀ aryl, C₃₋₇ cycloalkyl, 5-6 membered heteroaryl, and 4-7 membered heterocycloalkyl are each optionally substituted with 1, 2, or 3 substituents independently selected from OH, CN, amino, halo, C₁₋₆ alkyl, C₁₋₆ alkoxy, C₁₋₆ haloalkyl, and C₁₋₆ haloalkoxy;
each R^{e}, R^{e1}, R^{e2}, and R^{e3} is independently selected from H, D, C₁₋₄ alkyl, and CN;
n is 0, 1, 2, or 3; and
m is 0, 1 or 2.

2. A compound of Formula I: 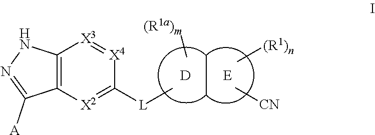
or a pharmaceutically acceptable salt thereof, wherein:
A is selected from Cy¹, Cy¹-C₁₋₄ alkyl-, halo, C₁₋₆ alkyl, C₂₋₆ alkenyl, C₂₋₆ alkynyl, C₁₋₆ haloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, C(═NR^{e})NR^{c}R^{d}, NR^{c}C(═NR^{e})NR^{c}R^{d}, NR^{c}R^{d}, NR^{c}C(O)R^{b}, NR^{c}C(O)OR^{a}, NR^{c}C(O)NR^{c}R^{d}, NR^{c}S(O)R^{b}, NR^{c}S(O)₂R^{b}, NR^{c}S(O)₂NR^{c}R^{d}, S(O)R^{b}, S(O)NR^{c}R^{d}, S(O)₂R^{b}, and S(O)₂NR^{c}R^{d}; wherein said C₁₋₆ alkyl, C₂₋₆ alkenyl, C₂₋₆ alkynyl, and C₁₋₆ haloalkyl of A are each optionally substituted with 1, 2, 3, 4, or 5 substituents independently selected from halo, C₁₋₆ alkyl, C₂₋₆ alkenyl, C₂₋₆ alkynyl, C₁₋₆ haloalkyl, C₃₋₇ cycloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, C(═NR^{e})NR^{c}R^{d}, NR^{c}C(═NR^{e})NR^{c}R^{d}, NR^{c}R^{d}, NR^{c}C(O)R^{b}, NR^{c}C(O)OR^{a}, NR^{c}C(O)NR^{c}R^{d}, NR^{c}S(O)R^{b}, NR^{c}S(O)₂R^{b}, NR^{c}S(O)₂NR^{c}R^{d}, S(O)R^{b}, S(O)NR^{c}R^{d}, S(O)₂R^{b}, and S(O)₂NR^{c}R^{d};
L is O, S, or NR^{N};
R^{N} is H or C₁₋₄ alkyl;
X² is N or CR²;
X³ is N or CR³;
X⁴ is N or CR⁴; wherein not more than two of X², X³, and X⁴ are simultaneously N;
Ring D is a C₄₋₇ cycloalkyl group fused with Ring E;
Ring E is phenyl or a 5- to 6-membered heteroaryl group, fused with Ring D;
Cy¹ is selected from C₆₋₁₀ aryl, C₃₋₁₀ cycloalkyl, 5-14 membered heteroaryl, and 4-14 membered heterocycloalkyl, each optionally substituted by 1, 2, 3, 4, or 5 substituents independently selected from halo, C₁₋₆ alkyl, C₂₋₆ alkenyl, C₂₋₆ alkynyl, C₁₋₆ haloalkyl, C₆₋₁₀ aryl, C₃₋₁₀ cycloalkyl, 5-14 membered heteroaryl, 4-14 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a}, SR^{a}, C(O)R^{b}, C(O)NR^{e}R^{d}, C(O)OR^{a}, OC(O)R^{b}, OC(O)NR^{c}R^{d}, NR^{c}R^{d}, NR^{c}C(O)R^{b}, NR^{c}C(O)OR^{a}, NR^{c}C(O)NR^{c}R^{d}, C(═NR^{e})R^{b}, C(═NR^{e})NR^{c}R^{d}, NR^{c}C(═NR^{e})NR^{c}R^{d}, NR^{c}S(O)R^{b}, NR^{c}S(O)₂R^{b}, NR^{c}S(O)₂NR^{c}R^{d}, S(O)R^{b}, S(O)NR^{e}R^{d}, S(O)₂R^{b}, and S(O)₂NR^{e}R^{d}, wherein said substituents C₁₋₆ alkyl, C₂₋₆ alkenyl, C₂₋₆ alkynyl, C₁₋₆ haloalkyl, C₆₋₁₀ aryl, C₃₋₁₀ cycloalkyl, 5-14 membered heteroaryl, 4-14 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl are each optionally substituted by 1, 2, or 3 further substituents independently selected from halo, C₁₋₆ alkyl, C₂₋₆ alkenyl, C₂₋₆ alkynyl, C₁₋₆ haloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)OR^{a}, NR^{c}C(O)NR^{c}R^{d}, C(═NR^{e})R^{b}, C(═NR^{e})NR^{c}R^{d}, NR^{c}C(═NR^{e})NR^{c}R^{d}, NR^{c}S(O)R^{b}, NR^{c}S(O)₂R^{b}, NR^{c}S(O)₂NR^{c}R^{d}, S(O)R^{b}, S(O)NR^{c}R^{d}, S(O)₂R^{b}, and S(O)₂NR^{c}R^{d};
each R¹ and R^{1a} is independently selected from H, halo, C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}; wherein said C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R¹ are each optionally substituted with 1, 2, 3, 4, or 5 substituents independently selected from halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};
or two R¹ groups together with the atoms to which they are attached form a C₅₋₇ cycloalkyl group which is optionally substituted with 1, 2, 3, 4, or 5 substituents independently selected from halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};
R² and R⁴ are each independently selected from H, halo, C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2}, wherein said C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R² and R⁴ are each optionally substituted with 1, 2, 3, 4, or 5 substituents independently selected from halo, C₁₋₆ alkyl, C₂₋₆ alkenyl, C₂₋₆ alkynyl, C₁₋₆ haloalkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2};
R³ is selected from H, halo, C₁₋₆ alkyl, C₂₋₆ alkenyl, C₂₋₆ alkynyl, C₁₋₆ haloalkyl, C₃₋₄ cycloalkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, C(═NR^{e3})R^{b3}, C(═NR^{e3})NR^{c3}R^{d3}, NR^{c3}C(═NR^{e3})NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3}, wherein said C₁₋₆ alkyl, C₂₋₆ alkenyl, C₂₋₆ alkynyl, C₁₋₆ haloalkyl, and C₃₋₄ cycloalkyl of R³ are each optionally substituted with 1, 2, or 3 substituents independently selected from halo, C₁₋₄ alkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, C(═NR^{e3})R^{b3}, C(═NR^{e3})NR^{c3}R^{d3}, NR^{c3}C(═NR^{e3})NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3};
each R^{a}, R^{b}, R^{c}, R^{d}, R^{a1}, R^{b1}, R^{c1}, R^{d1}, R^{a2}, R^{b2}, R^{c2}, and R^{d2} is independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl, wherein said C₁₋₆ alkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{a}, R^{b}, R^{c}, R^{d}, R^{a1}, R^{b1}, R^{c1}, R^{d1}, R^{a2}, R^{b2}, R^{c2}, and R^{d2} is optionally substituted with 1, 2, 3, 4, or 5 substituents independently selected from halo, C₁₋₄ alkyl, C₁₋₄haloalkyl, C₁₋₆ haloalkyl, C₂₋₆ alkenyl, C₂₋₆ alkynyl, CN, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)NR^{c3}R^{d3}, NR^{c3}C(O)OR^{a3}, C(═NR^{e3})NR^{c3}R^{d3}, NR^{c3}C(═NR^{e3})NR^{c3}R^{d3}, S(O)R^{b3}, S(O)NR^{c3}R^{d3}, S(O)₂R^{b3}, NR^{c3}S(O)₂R^{b3}, NR^{c3}S(O)₂NR^{c3}R^{d3}, and S(O)₂NR^{c3}R^{d3};
each R^{a3}, R^{b3}, R^{C3}, and R^{d3} is independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-6 membered heteroaryl, and 4-7 membered heterocycloalkyl, wherein said C₁₋₆ alkyl, C₁₋₆ haloalkyl, C₂₋₆ alkenyl, C₂₋₆ alkynyl, C₆₋₁₀ aryl, C₃₋₇ cycloalkyl, 5-6 membered heteroaryl, and 4-7 membered heterocycloalkyl are each optionally substituted with 1, 2, or 3 substituents independently selected from OH, CN, amino, halo, C₁₋₆ alkyl, C₁₋₆ alkoxy, C₁₋₆ haloalkyl, and C₁₋₆ haloalkoxy;
each R^{e}, R^{e1}, R^{e2}, and R^{e3} is independently selected from H, C₁₋₄ alkyl, and CN;
n is 0, 1, 2, or 3; and
m is 0, 1 or 2.

3. The compound of claim 1 or 2, or a pharmaceutically acceptable salt thereof, wherein X² is CR².

4. The compound of claim 1 or 2, or a pharmaceutically acceptable salt thereof, wherein X² is N.

5. The compound of any one of claims 1-4, or a pharmaceutically acceptable salt thereof, wherein X³ is CR³.

6. The compound of any one of claims 1-5, or a pharmaceutically acceptable salt thereof, wherein X⁴ is CR⁴.

7. The compound of any one of claims 1-5, or a pharmaceutically acceptable salt thereof, wherein X⁴ is N.

8. The compound of any one of claims 1-7, or a pharmaceutically acceptable salt thereof, wherein L is O or NR^{N}.

9. The compound of any one of claims 1-7, or a pharmaceutically acceptable salt thereof, wherein L is O.

10. The compound of any one of claims 1-7, or a pharmaceutically acceptable salt thereof, wherein L is NH.

11. The compound of any one of claims 1-7, or a pharmaceutically acceptable salt thereof, wherein L is NCH₃.

12. The compound of any one of claims 1-11, or a pharmaceutically acceptable salt thereof, wherein A is selected from Cy¹, halo, C₁₋₆ alkyl, C₁₋₆ haloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)OR^{a}, NR^{c}C(O)NR^{c}R^{d}, NR^{c}S(O)₂R^{b}, NR^{c}S(O)₂NR^{c}R^{d}, S(O)₂R^{b}, and S(O)₂NR^{c}R^{d}; wherein said C₁₋₆ alkyl and C₁₋₆ haloalkyl of A are each optionally substituted with 1, 2, 3, 4, or 5 substituents independently selected from halo, C₁₋₆ alkyl, C₁₋₆ haloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)OR^{a}, NR^{c}C(O)NR^{c}R^{d}, NR^{c}S(O)₂R^{b}, NR^{c}S(O)₂NR^{c}R^{d}, S(O)₂R^{b}, and S(O)₂NR^{c}R^{d}.

13. The compound of any one of claims 1-11, or a pharmaceutically acceptable salt thereof, wherein A is selected from Cy¹, halo, and C₁₋₆ alkyl.

14. The compound of any one of claims 1-11, or a pharmaceutically acceptable salt thereof, wherein A is selected from Cy¹, Cy¹-C₁₋₄ alkyl-, Cy¹-C₂₋₄ alkenyl-, halo, C₁₋₆ alkyl, C₁₋₆ haloalkyl, C₂₋₆ alkenyl, and OR^{a}.

15. The compound of any one of claims 1-11, or a pharmaceutically acceptable salt thereof, wherein A is selected from halo and C₁₋₆ alkyl.

16. The compound of any one of claims 1-11, or a pharmaceutically acceptable salt thereof, wherein A is selected from methyl and iodide.

17. The compound of any one of claims 1-11, or a pharmaceutically acceptable salt thereof, wherein A is selected from cyclopropylmethyl, styryl, methyl, bromide, chloride, iodide, CF₃, prop-1-en-1-yl, and methoxy.

18. The compound of any one of claims 1-17, or a pharmaceutically acceptable salt thereof, wherein Cy¹ is 5-6 membered heteroaryl optionally substituted by 1 or 2 substituents independently selected from halo, C₁₋₆ alkyl, C₁₋₆ haloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)OR^{a}, NR^{c}C(O)NR^{c}R^{d}, NR^{c}S(O)₂R^{b}, NR^{c}S(O)₂NR^{c}R^{d}, S(O)₂R^{b}, and S(O)₂NR^{c}R^{d}.

19. The compound of any one of claims 1-17, or a pharmaceutically acceptable salt thereof, wherein Cy¹ is C₃₋₆ cycloalkyl, phenyl, or 5-6 membered heteroaryl, each optionally substituted by 1 or 2 substituents independently selected from 4-14 membered heterocycloalkyl, halo, C₁₋₆ alkyl, C₁₋₆ haloalkyl, CN, NO₂, OR^{a}, SR^{a}, C(O)R^{b}, C(O)NR^{c}R^{d}, C(O)OR^{a}, C(O)N(R^{c})OR^{a}, OC(O)R^{b}, OC(O)NR^{c}R^{d}, NR^{c}R^{d}, NR^{c}C(O)R^{b}, NR^{c}C(O)OR^{a}, NR^{c}C(O)NR^{c}R^{d}, NR^{c}S(O)₂R^{b}, NR^{c}S(O)₂NR^{c}R^{d}, S(O)₂R^{b}, and S(O)₂NR^{c}R^{d}.

20. The compound of any one of claims 1-17, or a pharmaceutically acceptable salt thereof, wherein Cy¹ is selected from isoxazolyl, oxazolyl, pyrazolyl, and furanyl, each of which is optionally substituted by 1 or 2 substituents independently selected from halo, C₁₋₆ alkyl, C₁₋₆ haloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)OR^{a}, NR^{c}C(O)NR^{c}R^{d}, NR^{c}S(O)₂R^{b}, NR^{c}S(O)₂NR^{c}R^{d}, S(O)₂R^{b}, and S(O)₂NR^{c}R^{d}.

21. The compound of any one of claims 1-17, or a pharmaceutically acceptable salt thereof, wherein Cy¹ is selected from phenyl, pyridinyl, isoxazolyl, oxazolyl, pyrazolyl, furanyl, thiazolyl, cyclohexyl, oxo-1,2-dihydropyridinyl, cyclohex-1-en-1-yl, 1H,2′H-[3,6′-biindazol]-yl, benzo[d]thiazolyl, 1H-indolyl, 6-oxo-1,6-dihydropyridin-3-yl, cyclopent-1-en-1-yl, benzo[d]thiazolyl, benzo[d][1,3]dioxolyl, 2-oxoindolinyl, 1H,2′H-[3,5′-biindazol]-5-yl, 2,3-dihydrobenzo[b][1,4]dioxinyl, 1,4-dioxaspiro[4.5]dec-7-enyl, 3,6-dihydro-2H-pyran-4-yl, 1,2,3,6-tetrahydropyridin-4-yl, 5,6-dihydro-4H-pyrrolo[1,2-b]pyrazolyl, 2-oxo-1,2-dihydropyridin-4-yl, and 1,2-oxazolyl, each of which is optionally substituted by 1 or 2 substituents independently selected from 5-6 membered heterocycloalkyl, C₃₋₆ cycloalkyl, halo, C₁₋₆ alkyl, C₁₋₆ haloalkyl, CN, NO₂, OR^{a}, SR^{a}, C(O)R^{b}, C(O)NR^{C}R^{d}, C(O)OR^{a}, OC(O)R^{b}, OC(O)NR^{c}R^{d}, NR^{c}R^{d}, NR^{c}C(O)R^{b}, NR^{c}C(O)OR^{a}, NR^{c}C(O)NR^{c}R^{d}, NR^{c}S(O)₂R^{b}, NR^{c}S(O)₂NR^{c}R^{d}, S(O)₂R^{b}, and S(O)₂NR^{c}R^{d}.

22. The compound of any one of claims 1-17, or a pharmaceutically acceptable salt thereof, wherein Cy¹ is selected from isoxazol-4-yl, oxazol-5-yl, 1-(difluoromethyl)-1H-pyrazol-4-yl, and furan-3-yl.

23. The compound of any one of claims 1-17, or a pharmaceutically acceptable salt thereof, wherein Cy¹ is selected from isoxazol-4-yl, oxazol-5-yl, 1-(difluoromethyl)-1H-pyrazol-4-yl, furan-3-yl, 4-carboxyphenyl, thiazol-5-yl, 1H-2-yl, 1-methyl-1H-2-yl, 2-methyloxazol-5-yl, 1H-pyrazol-5-yl, 3-methylisothiazol-5-yl, pyrazin-2-yl, 2-morpholinopyridin-4-yl, 2-methoxypyridin-4-yl, cyclopropyl, cyclohexyl, 1-methyl-2-oxo-1,2-dihydropyridin-3-yl, 2′-methyl-1H,2′H-[3,6′-biindazol]-5-yl, 3-(methylsulfonyl)phenyl, 3,5-dimethoxyphenyl, benzo[d]thiazol-6-yl, 1H-indol-6-yl, 1-methyl-6-oxo-1,6-dihydropyridin-3-yl, 4-cyanophenyl, pyridin-4-yl, cyclopent-1-en-1-yl, 3-carboxy-4-fluorophenyl, benzo[d]thiazol-5-yl, 3-(difluoromethyl)phenyl, 3-(methoxycarbamoyl)phenyl, 4-nitrophenyl, 3,4-dimethoxyphenyl, 4-morpholinophenyl, 4-methoxy-3-methylphenyl, 4-(methylsulfonyl)phenyl, 5-cyclopropylpyridin-3-yl, benzo[d][1,3]dioxol-5-yl, 1H-indol-6-yl, 1-(tert-butoxycarbonyl)-1H-pyrrol-2-yl, 4-(morpholine-4-carbonyl)phenyl, 2-oxoindolin-6-yl, 2′-methyl-1H,2′H-[3,5′-biindazol]-5-yl, 2,3-dihydrobenzo[b][1,4]dioxin-6-yl, 3-acetamidophenyl, 3-(dimethylcarbamoyl)phenyl, 1,4-dioxaspiro[4.5]dec-7-en-8-yl, 3,6-dihydro-2H-pyran-4-yl, 3-cyanophenyl, 2-methylpyridin-4-yl, 6-cyanopyridin-3-yl, 4-methoxyphenyl, 1-methyl-1,2,3,6-tetrahydropyridin-4-yl, 4-bromophenyl, 5,6-dihydro-4H-pyrrolo[1,2-b]pyrazol-3-yl, 1-methyl-2-oxo-1,2-dihydropyridin-4-yl, pyridin-3-yl, 5-methylpyridin-3-yl, 2-ethylpyridin-4-yl, 2-methoxypyridin-4-yl, and 1,2-oxazol-4-yl.

24. The compound of any one of claims 1-23, or a pharmaceutically acceptable salt thereof, wherein Ring D is cyclopentyl or cyclohexyl fused with Ring E.

25. The compound of any one of claims 1-23, or a pharmaceutically acceptable salt thereof, wherein Ring D is cyclopentyl, cyclohexyl, or cycloheptyl, each of which is fused with Ring E.

26. The compound of any one of claims 1-23, or a pharmaceutically acceptable salt thereof, wherein Ring D is cyclopentyl fused with Ring E.

27. The compound of any one of claims 1-23, or a pharmaceutically acceptable salt thereof, wherein Ring D is cyclohexyl fused with Ring E.

28. The compound of any one of claims 1-23, or a pharmaceutically acceptable salt thereof, wherein Ring D is a tetrahydropyranyl group fused with Ring E.

29. The compound of any one of claims 1-28, or a pharmaceutically acceptable salt thereof, wherein Ring E is phenyl or a 6-membered heteroaryl group, fused with Ring D.

30. The compound of any one of claims 1-28, or a pharmaceutically acceptable salt thereof, wherein Ring E is phenyl or a 5-6 membered heteroaryl group, each of which is fused with Ring D.

31. The compound of any one of claims 1-28, or a pharmaceutically acceptable salt thereof, wherein Ring E is phenyl fused with Ring D.

32. The compound of any one of claims 1-28, or a pharmaceutically acceptable salt thereof, wherein Ring E is pyridinyl fused with Ring D.

33. The compound of any one of claims 1-28, or a pharmaceutically acceptable salt thereof, wherein Ring E is phenyl, pyridinyl, pyridazinyl, oxazolyl, thiazolyl, or pyrazinyl, each of which is fused with Ring D.

34. The compound of any one of claims 1-33, or a pharmaceutically acceptable salt thereof, wherein R^{1a} is H.

35. The compound of any one of claims 1-33, or a pharmaceutically acceptable salt thereof, wherein R^{1a} is H, D, F, or methyl.

36. The compound of any one of claims 1-33, or a pharmaceutically acceptable salt thereof, wherein two R^{1a} groups together with the atoms to which they are attached form a cyclopropyl group.

37. The compound of any one of claims 1-36, or a pharmaceutically acceptable salt thereof, wherein each R¹ is independently selected from H, halo, and C₁₋₆ alkyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, NR^{c1}S(O)₂NR^{c1}R^{d1}, S(O)₂R^{b1}, and S(O)₂NR^{c1}R^{d1}.

38. The compound of any one of claims 1-36, or a pharmaceutically acceptable salt thereof, wherein each R¹ is independently selected from halo and C₁₋₆ alkyl.

39. The compound of any one of claims 1-36, or a pharmaceutically acceptable salt thereof, wherein each R¹ is independently selected from F and methyl.

40. The compound of any one of claims 1-36, or a pharmaceutically acceptable salt thereof, wherein each R¹ is independently selected from F, Cl, Br, methyl, CF₃, OCH₃, and CHF₂.

41. The compound of any one of claims 1-40, or a pharmaceutically acceptable salt thereof, wherein R² is H.

42. The compound of any one of claims 1-41, or a pharmaceutically acceptable salt thereof, wherein R³ is H.

43. The compound of any one of claims 1-42, or a pharmaceutically acceptable salt thereof, wherein R⁴ is H.

44. The compound of any one of claims 1-43, or a pharmaceutically acceptable salt thereof, wherein m is 0.

45. The compound of any one of claims 1-43, or a pharmaceutically acceptable salt thereof, wherein m is 2.

46. The compound of any one of claims 1-45, or a pharmaceutically acceptable salt thereof, wherein n is 0 or 1.

47. The compound of any one of claims 1-45, or a pharmaceutically acceptable salt thereof, wherein n is 0, 1, or 2.

48. The compound of any one of claims 1-23 and 34-47, wherein the compound is of Formula II: 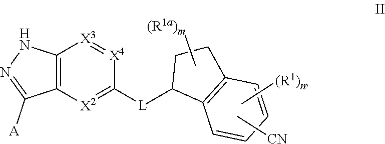
or a pharmaceutically acceptable salt thereof.

49. The compound of any one of claims 1, 2, 8-23, 37-40, and 44-47, wherein the compound is of Formula III: 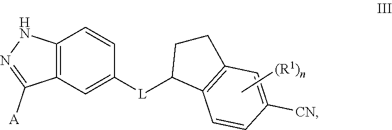
or a pharmaceutically acceptable salt thereof.

50. The compound of any one of claims 1-23 and 34-47, wherein the compound is of Formula IVa or Formula IVb: 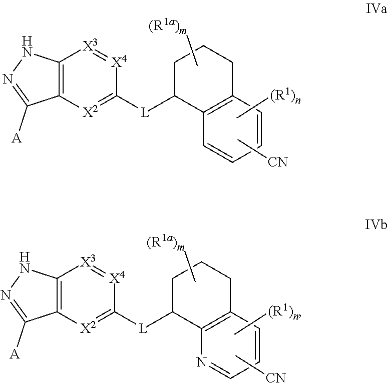
or a pharmaceutically acceptable salt thereof.

51. The compound of any one of claims 1, 2, 8-23, 37-40, and 44-47, wherein the compound is of Formula Va or Formula Vb: 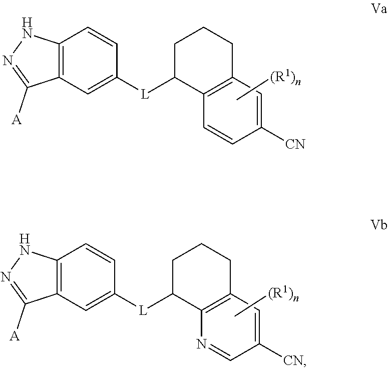
or a pharmaceutically acceptable salt thereof.

52. The compound of any one of claims 1, 2, 8-23, and 29-47, wherein the compound is of Formula VIa or Formula VIb: 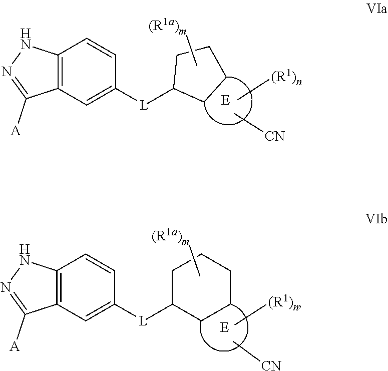
or a pharmaceutically acceptable salt thereof.

53. The compound of claim 1 or 2, or a pharmaceutically acceptable salt thereof, wherein:
A is selected from Cy¹, halo, and C₁₋₆ alkyl;
L is O or NH;
X² is CR²;
X³ is CR³;
X⁴ is N or CR⁴;
Ring D is cyclopentyl or cyclohexyl group fused with Ring E;
Ring E is phenyl or a 6-membered heteroaryl group, fused with Ring D;
Cy¹ is 5-6 membered heteroaryl optionally substituted by 1 or 2 substituents independently selected from halo, C₁₋₆ alkyl, C₁₋₆ haloalkyl, CN, NO₂, OR^{a}, SR^{a}, C(O)R^{b}, C(O)NR^{c}R^{d}, C(O)OR^{a}, OC(O)R^{b}, OC(O)NR^{c}R^{d}, NR^{c}R^{d}, NR^{c}C(O)R^{b}, NR^{c}C(O)OR^{a}, NR^{c}C(O)NR^{c}R^{d}, NR^{c}S(O)₂R^{b}, NR^{c}S(O)₂NR^{c}R^{d}, S(O)₂R^{b}, and S(O)₂NR^{c}R^{d};
each R¹ and R^{1a} is independently selected from H, halo, C₁₋₆ alkyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, NR^{c1}S(O)₂NR^{c1}R^{d1}, S(O)₂R^{b1}, and S(O)₂NR^{c1}R^{d1};
R², R³, and R⁴ are each H;
each R^{a}, R^{b}, R^{c}, R^{d}, R^{a1}, R^{b1}, R^{c1}, and R^{d1} is independently selected from H, C₁₋₆ alkyl, and C₁₋₆ haloalkyl, wherein said C₁₋₆ alkyl of R^{a}, R^{b}, R^{c}, R^{d}, R^{a1}, R^{b1}, R^{c1}, and R^{d1} is optionally substituted with 1, 2, 3, 4, or 5 substituents independently selected from halo, C₁₋₄ alkyl, C₁₋₄ haloalkyl, C₁₋₆ haloalkyl, CN, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)NR^{c3}R^{d3}, NR^{c3}C(O)OR^{a3}, S(O)₂R^{b3}, NR^{c3}S(O)₂R^{b3}, NR^{c3}S(O)₂NR^{c3}R^{d3}, and S(O)₂NR^{c3}R^{d3};
each R^{a3}, R^{b3}, R^{c3}, and R^{d3} is independently selected from H, C₁₋₆ alkyl, and C₁₋₆ haloalkyl, wherein said C₁₋₆ alkyl, and C₁₋₆ haloalkyl are each optionally substituted with 1, 2, or 3 substituents independently selected from OH, CN, amino, halo, C₁₋₆ alkyl, C₁₋₆ alkoxy, C₁₋₆ haloalkyl, and C₁₋₆ haloalkoxy;
n is 0, or 1; and
m is 0.

54. The compound of claim 1, or a pharmaceutically acceptable salt thereof, wherein:
A is selected from Cy¹, Cy¹-C₁₋₄ alkyl-, Cy¹-C₂₋₄ alkenyl-, halo, C₁₋₆ alkyl, C₁₋₆ haloalkyl, C₂₋₆ alkenyl, and OR^{a};
L is O, NH, or NCH₃;
X² is N or CR²;
X³ is CR³;
X⁴ is N or CR⁴;
Ring D is cyclopentyl, cyclohexyl, cycloheptyl, or tetrahydropyranyl, each of which is fused with Ring E;
Ring E is phenyl or a 5-6 membered heteroaryl group, each of which is fused with Ring D;
Cy¹ is C₃₋₆ cycloalkyl, phenyl, or 5-6 membered heteroaryl, each optionally substituted by 1 or 2 substituents independently selected from 4-14 membered heterocycloalkyl, halo, C₁₋₆ alkyl, C₁₋₆ haloalkyl, CN, NO₂, OR^{a}, SR^{a}, C(O)R^{b}, C(O)NR^{c}R^{d}, C(O)OR^{a}, C(O)N(R^{c})OR^{a}, OC(O)R^{b}, OC(O)NR^{c}R^{d}, NR^{c}R^{d}, NR^{c}C(O)R^{b}, NR^{c}C(O)OR^{a}, NR^{c}C(O)NR^{c}R^{d}, NR^{c}S(O)₂R^{b}, NR^{c}S(O)₂NR^{c}R^{d}, S(O)₂R^{b}, and S(O)₂NR^{c}R^{d};
each R¹ and R^{1a} is independently selected from H, D, halo, C₁₋₆ alkyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, NR^{c1}S(O)₂NR^{c1}R^{d1}, S(O)₂R^{b1}, and S(O)₂NR^{c1}R^{d1};
or two R^{1a} groups together with the atoms to which they are attached form a C₃₋₆ cycloalkyl group;
R², R³, and R⁴ are each H;
each R^{a}, R^{b}, R^{c}, R^{d}, R^{a1}, R^{b1}, R^{c1}, and R^{d1} is independently selected from H, C₁₋₆ alkyl, and C₁₋₆ haloalkyl, wherein said C₁₋₆ alkyl of R^{a}, R^{b}, R^{c}, R^{d}, R^{a1}, R^{b1}, R^{c1}, and R^{d1} is optionally substituted with 1, 2, 3, 4, or 5 substituents independently selected from halo, C₁₋₄ alkyl, C₁₋₄ haloalkyl, C₁₋₆ haloalkyl, CN, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)NR^{c3}R^{d3}, NR^{c3}C(O)OR^{a3}, S(O)₂R^{b3}, NR^{c3}S(O)₂R^{b3}, NR^{c3}S(O)₂NR^{c3}R^{d3}, and S(O)₂NR^{c3}R^{d3};
each R^{a3}, R^{b3}, R^{c3}, and R^{d3} is independently selected from H, C₁₋₆ alkyl, and C₁₋₆ haloalkyl, wherein said C₁₋₆ alkyl, and C₁₋₆ haloalkyl are each optionally substituted with 1, 2, or 3 substituents independently selected from OH, CN, amino, halo, C₁₋₆ alkyl, C₁₋₆ alkoxy, C₁₋₆ haloalkyl, and C₁₋₆ haloalkoxy;
n is 0, 1, or 2; and
m is 0, 1, or 2.

55. The compound of claim 1 or 2, which is selected from:
(S)-1-((3-Methyl-1H-indazol-5-yl)oxy)-2,3-dihydro-1H-indene-5-carbonitrile;
(R)-1-((3-Methyl-1H-indazol-5-yl)oxy)-2,3-dihydro-1H-indene-5-carbonitrile;
(S)-8-((3-(Isoxazol-4-yl)-1H-indazol-5-yl)oxy)-5,6,7,8-tetrahydroquinoline-3-carbonitrile;
(R)-8-((3-(Isoxazol-4-yl)-1H-indazol-5-yl)oxy)-5,6,7,8-tetrahydroquinoline-3-carbonitrile;
(S)-1-((3-(Oxazol-5-yl)-1H-indazol-5-yl)oxy)-2,3-dihydro-1H-indene-5-carbonitrile;
(R)-1-((3-(Oxazol-5-yl)-1H-indazol-5-yl)oxy)-2,3-dihydro-1H-indene-5-carbonitrile;
(S)-1-((3-(1-(Difluoromethyl)-1H-pyrazol-4-yl)-1H-indazol-5-yl)oxy)-2,3-dihydro-1H-indene-5-carbonitrile;
(R)-1-((3-(1-(Difluoromethyl)-1H-pyrazol-4-yl)-1H-indazol-5-yl)oxy)-2,3-dihydro-1H-indene-5-carbonitrile;
(S)-1-((3-(Isoxazol-4-yl)-1H-indazol-5-yl)oxy)-2,3-dihydro-1H-indene-5-carbonitrile;
(S)-1-((3-(Isoxazol-4-yl)-1H-indazol-5-yl)oxy)-2,3-dihydro-1H-indene-5-carbonitrile;
(S)-5-((3-(Isoxazol-4-yl)-1H-indazol-5-yl)amino)-5,6,7,8-tetrahydronaphthalene-2-carbonitrile;
(R)-5-((3-(Isoxazol-4-yl)-1H-indazol-5-yl)amino)-5,6,7,8-tetrahydronaphthalene-2-carbonitrile;
(S)-8-((3-(Isoxazol-4-yl)-1H-indazol-5-yl)amino)-5,6,7,8-tetrahydroquinoline-3-carbonitrile;
(R)-8-((3-(Isoxazol-4-yl)-1H-indazol-5-yl)amino)-5,6,7,8-tetrahydroquinoline-3-carbonitrile;
(S)-1-((3-(Isoxazol-4-yl)-1H-indazol-5-yl)amino)-2,3-dihydro-1H-indene-5-carbonitrile;
(R)-1-((3-(Isoxazol-4-yl)-1H-indazol-5-yl)amino)-2,3-dihydro-1H-indene-5-carbonitrile;
(S)-1-((3-(Oxazol-5-yl)-1H-indazol-5-yl)amino)-2,3-dihydro-H-indene-5-carbonitrile;
(R)-1-((3-(Oxazol-5-yl)-1H-indazol-5-yl)amino)-2,3-dihydro-1H-indene-5-carbonitrile;
1-((3-(Furan-3-yl)-1H-indazol-5-yl)amino)-2,3-dihydro-1H-indene-5-carbonitrile;
(S)-1-((3-(Furan-3-yl)-1H-indazol-5-yl)amino)-2,3-dihydro-1H-indene-5-carbonitrile;
(R)-1-((3-(Furan-3-yl)-1H-indazol-5-yl)amino)-2,3-dihydro-1H-indene-5-carbonitrile (S)-1-((3-(1-(Difluoromethyl)-1H-pyrazol-4-yl)-1H-indazol-5-yl)amino)-2,3-dihydro-1H-indene-5-carbonitrile;
(R)-1-((3-(1-(Difluoromethyl)-1H-pyrazol-4-yl)-1H-indazol-5-yl)amino)-2,3-dihydro-1H-indene-5-carbonitrile;
(S)-1-((3-Iodo-1H-indazol-5-yl)oxy)-2,3-dihydro-1H-indene-5-carbonitrile;
(S)-7-methyl-1-((3-(oxazol-5-yl)-1H-indazol-5-yl)oxy)-2,3-dihydro-1H-indene-5-carbonitrile;
(R)-7-methyl-1-((3-(oxazol-5-yl)-1H-indazol-5-yl)oxy)-2,3-dihydro-1H-indene-5-carbonitrile;
(S)-5-((3-iodo-1H-indazol-5-yl)amino)-5,6,7,8-tetrahydronaphthalene-2-carbonitrile;
(R)-5-((3-iodo-1H-indazol-5-yl)amino)-5,6,7,8-tetrahydronaphthalene-2-carbonitrile;
(S)-8-((3-(Oxazol-5-yl)-1H-indazol-5-yl)oxy)-5,6,7,8-tetrahydroquinoline-3-carbonitrile;
(R)-8-((3-(Oxazol-5-yl)-1H-indazol-5-yl)oxy)-5,6,7,8-tetrahydroquinoline-3-carbonitrile;
(S)-5-((3-(Isoxazol-4-yl)-1H-indazol-5-yl)oxy)-5,6,7,8-tetrahydronaphthalene-2-carbonitrile;
(R)-5-((3-(Isoxazol-4-yl)-1H-indazol-5-yl)oxy)-5,6,7,8-tetrahydronaphthalene-2-carbonitrile;
(S)-5-((3-(Oxazol-5-yl)-1H-indazol-5-yl)oxy)-5,6,7,8-tetrahydronaphthalene-2-carbonitrile;
(R)-5-((3-(Oxazol-5-yl)-1H-indazol-5-yl)oxy)-5,6,7,8-tetrahydronaphthalene-2-carbonitrile;
(S)-5-((3-Methyl-1H-indazol-5-yl)oxy)-5,6,7,8-tetrahydronaphthalene-2-carbonitrile;
(R)-5-((3-Methyl-1H-indazol-5-yl)oxy)-5,6,7,8-tetrahydronaphthalene-2-carbonitrile;
(S)-1-((3-(Oxazol-5-yl)-1H-pyrazolo[3,4-c]pyridin-5-yl)oxy)-2,3-dihydro-1H-indene-5-carbonitrile;
(R)-1-((3-(Oxazol-5-yl)-1H-pyrazolo[3,4-c]pyridin-5-yl)oxy)-2,3-dihydro-1H-indene-5-carbonitrile;
(S)-1-((3-(Oxazol-5-yl)-1H-pyrazolo[3,4-c]pyridin-5-yl)amino)-2,3-dihydro-1H-indene-5-carbonitrile
(R)-1-((3-(Oxazol-5-yl)-1H-pyrazolo[3,4-c]pyridin-5-yl)amino)-2,3-dihydro-1H-indene-5-carbonitrile
(S)-4-Methyl-1-((3-(oxazol-5-yl)-1H-indazol-5-yl)oxy)-2,3-dihydro-1H-indene-5-carbonitrile;
(R)-4-Methyl-1-((3-(oxazol-5-yl)-1H-indazol-5-yl)oxy)-2,3-dihydro-1H-indene-5-carbonitrile;
(S)-6-Fluoro-1-((3-(oxazol-5-yl)-1H-indazol-5-yl)oxy)-2,3-dihydro-1H-indene-5-carbonitrile;
(R)-6-Fluoro-1-((3-(oxazol-5-yl)-1H-indazol-5-yl)oxy)-2,3-dihydro-1H-indene-5-carbonitrile;
(S)-4-Fluoro-1-((3-(oxazol-5-yl)-1H-indazol-5-yl)oxy)-2,3-dihydro-1H-indene-5-carbonitrile;
(R)-4-Fluoro-1-((3-(oxazol-5-yl)-1H-indazol-5-yl)oxy)-2,3-dihydro-1H-indene-5-carbonitrile;
(S)-6-Fluoro-1-((3-(oxazol-5-yl)-1H-indazol-5-yl)amino)-2,3-dihydro-1H-indene-5-carbonitrile;
(R)-6-Fluoro-1-((3-(oxazol-5-yl)-1H-indazol-5-yl)amino)-2,3-dihydro-1H-indene-5-carbonitrile;
(S)-4-Fluoro-1-((3-(oxazol-5-yl)-1H-indazol-5-yl)amino)-2,3-dihydro-1H-indene-5-carbonitrile;
(R)-4-Fluoro-1-((3-(oxazol-5-yl)-1H-indazol-5-yl)amino)-2,3-dihydro-1H-indene-5-carbonitrile;
(S)-4-Methyl-1-((3-(oxazol-5-yl)-1H-indazol-5-yl)amino)-2,3-dihydro-1H-indene-5-carbonitrile;
(R)-4-Methyl-1-((3-(oxazol-5-yl)-1H-indazol-5-yl)amino)-2,3-dihydro-1H-indene-5-carbonitrile;
(S)-6-Methyl-1-((3-(oxazol-5-yl)-1H-indazol-5-yl)amino)-2,3-dihydro-1H-indene-5-carbonitrile; and
(R)-6-Methyl-1-((3-(oxazol-5-yl)-1H-indazol-5-yl)amino)-2,3-dihydro-1H-indene-5-carbonitrile;
or a pharmaceutically acceptable salt of any of the aforementioned.

56. The compound of claim 1, which is selected from:
6-Methyl-1-((3-(oxazol-5-yl)-1H-indazol-5-yl)oxy)-2,3-dihydro-1H-indene-5-carbonitrile;
1-((3-(Oxazol-5-yl)-1H-pyrazolo[4,3-b]pyridin-5-yl)amino)-2,3-dihydro-1H-indene-5-carbonitrile;
1-((3-(Oxazol-5-yl)-1H-pyrazolo[4,3-b]pyridin-5-yl)oxy)-2,3-dihydro-1H-indene-5-carbonitrile;
5-((3-(Oxazol-5-yl)-1H-pyrazolo[3,4-c]pyridin-5-yl)oxy)-5,6,7,8-tetrahydronaphthalene-2-carbonitrile;
5-((3-(Oxazol-5-yl)-1H-pyrazolo[3,4-c]pyridin-5-yl)amino)-5,6,7,8-tetrahydronaphthalene-2-carbonitrile;
6,6-Difluoro-7-((3-(oxazol-5-yl)-1H-indazol-5-yl)amino)-6,7-dihydro-5H-cyclopenta[b]pyridine-3-carbonitrile;
4-((3-(Oxazol-5-yl)-1H-indazol-5-yl)amino)-4,5,6,7-tetrahydrobenzo[d]oxazole-2-carbonitrile;
4-((3-(Oxazol-5-yl)-1H-indazol-5-yl)amino)-5,6-dihydro-4H-cyclopenta[d]oxazole-2-carbonitrile;
3-((3-(Oxazol-5-yl)-1H-indazol-5-yl)oxy)-2,3-dihydro-1H-indene-4-carbonitrile;
5-((3-(2-Morpholinopyridin-4-yl)-1H-indazol-5-yl)amino)-5,6,7,8-tetrahydronaphthalene-2-carbonitrile;
5-((3-(2-Morpholinopyridin-4-yl)-1H-indazol-5-yl)oxy)-5,6,7,8-tetrahydronaphthalene-2-carbonitrile;
1-((3-(2-Methoxypyridin-4-yl)-1H-indazol-5-yl)amino)-2,3-dihydro-1H-indene-5-carbonitrile;
7-((3-(Oxazol-5-yl)-1H-indazol-5-yl)oxy)-6,7-dihydro-5H-cyclopenta[c]pyridazine-3-carbonitrile;
2,2-Difluoro-1-((3-(oxazol-5-yl)-1H-indazol-5-yl)amino)-2,3-dihydro-1H-indene-5-carbonitrile;
5-((3-Methyl-1H-indazol-5-yl)amino)-5,6,7,8-tetrahydronaphthalene-2-carbonitrile;
5-((3-(Thiazol-5-yl)-1H-indazol-5-yl)amino)-5,6,7,8-tetrahydronaphthalene-2-carbonitrile;
5-((3-Cyclohexyl-1H-indazol-5-yl)amino)-5,6,7,8-tetrahydronaphthalene-2-carbonitrile;
5-((3-(1-Methyl-2-oxo-1,2-dihydropyridin-3-yl)-1H-indazol-5-yl)amino)-5,6,7,8-tetrahydronaphthalene-2-carbonitrile;
7-((3-(Oxazol-5-yl)-1H-indazol-5-yl)amino)-4,5,6,7-tetrahydrobenzo[d]thiazole-2-carbonitrile;
6-((3-(Oxazol-5-yl)-1H-indazol-5-yl)amino)-4b,5,5a,6-tetrahydrocyclopropa[3,4]cyclopenta[1,2-b]pyridine-3-carbonitrile;
8-((3-(1-(Difluoromethyl)-1H-pyrazol-4-yl)-1H-indazol-5-yl)amino)-5,6,7,8-tetrahydroquinoline-3-carbonitrile;
1-((3-(1-(Difluoromethyl)-1H-pyrazol-4-yl)-1H-indazol-5-yl)oxy)-4-methyl-2,3-dihydro-1H-indene-5-carbonitrile;
1-((3-(1-(Difluoromethyl)-1H-pyrazol-4-yl)-1H-indazol-5-yl)oxy)-4-fluoro-2,3-dihydro-1H-indene-5-carbonitrile;
1-((3-(1-(Difluoromethyl)-1H-pyrazol-4-yl)-1H-indazol-5-yl)amino)-4-methyl-2,3-dihydro-1H-indene-5-carbonitrile;
1-((3-(1-(Difluoromethyl)-1H-pyrazol-4-yl)-1H-indazol-5-yl)amino)-4-fluoro-2,3-dihydro-1H-indene-5-carbonitrile;
5-((3-(Cyclohex-1-en-1-yl)-1H-indazol-5-yl)amino)-5,6,7,8-tetrahydronaphthalene-2-carbonitrile;
4-(5-((6-Cyano-1,2,3,4-tetrahydronaphthalen-1-yl)amino)-1H-indazol-3-yl)benzoic acid;
5-((2′-Methyl-1H,2′H-[3,6′-biindazol]-5-yl)amino)-5,6,7,8-tetrahydronaphthalene-2-carbonitrile;
5-((3-(3-(Methylsulfonyl)phenyl)-1H-indazol-5-yl)amino)-5,6,7,8-tetrahydronaphthalene-2-carbonitrile;
5-((3-(3,5-Dimethoxyphenyl)-1H-indazol-5-yl)amino)-5,6,7,8-tetrahydronaphthalene-2-carbonitrile;
5-((3-(Benzo[d]thiazol-6-yl)-1H-indazol-5-yl)amino)-5,6,7,8-tetrahydronaphthalene-2-carbonitrile;
5-((3-(2-Methyloxazol-5-yl)-1H-indazol-5-yl)amino)-5,6,7,8-tetrahydronaphthalene-2-carbonitrile;
5-((3-(1H-Indol-6-yl)-1H-indazol-5-yl)amino)-5,6,7,8-tetrahydronaphthalene-2-carbonitrile;
5-((3-(1-Methyl-6-oxo-1,6-dihydropyridin-3-yl)-1H-indazol-5-yl)amino)-5,6,7,8-tetrahydronaphthalene-2-carbonitrile;
5-((3-(4-Cyanophenyl)-1H-indazol-5-yl)amino)-5,6,7,8-tetrahydronaphthalene-2-carbonitrile;
5-((3-(Pyridin-4-yl)-1H-indazol-5-yl)amino)-5,6,7,8-tetrahydronaphthalene-2-carbonitrile;
5-((3-(Cyclopent-1-en-1-yl)-1H-indazol-5-yl)amino)-5,6,7,8-tetrahydronaphthalene-2-carbonitrile;
5-(5-((6-Cyano-1,2,3,4-tetrahydronaphthalen-1-yl)amino)-1H-indazol-3-yl)-2-fluorobenzoic acid;
5-((3-(Benzo[d]thiazol-5-yl)-1H-indazol-5-yl)amino)-5,6,7,8-tetrahydronaphthalene-2-carbonitrile;
5-((3-(3-(Difluoromethyl)phenyl)-1H-indazol-5-yl)amino)-5,6,7,8-tetrahydronaphthalene-2-carbonitrile;
3-(5-((6-Cyano-1,2,3,4-tetrahydronaphthalen-1-yl)amino)-1H-indazol-3-yl)-N-methoxybenzamide;
5-((3-(4-Nitrophenyl)-1H-indazol-5-yl)amino)-5,6,7,8-tetrahydronaphthalene-2-carbonitrile;
5-((3-(3,4-Dimethoxyphenyl)-1H-indazol-5-yl)amino)-5,6,7,8-tetrahydronaphthalene-2-carbonitrile;
5-((3-(4-Morpholinophenyl)-1H-indazol-5-yl)amino)-5,6,7,8-tetrahydronaphthalene-2-carbonitrile;
5-((3-(4-Methoxy-3-methylphenyl)-1H-indazol-5-yl)amino)-5,6,7,8-tetrahydronaphthalene-2-carbonitrile;
5-((3-(4-(Methylsulfonyl)phenyl)-1H-indazol-5-yl)amino)-5,6,7,8-tetrahydronaphthalene-2-carbonitrile;
(E)-5-((3-(Prop-1-en-1-yl)-1H-indazol-5-yl)amino)-5,6,7,8-tetrahydronaphthalene-2-carbonitrile;
5-((3-(5-Cyclopropylpyridin-3-yl)-1H-indazol-5-yl)amino)-5,6,7,8-tetrahydronaphthalene-2-carbonitrile;
5-((3-(Benzo[d][1,3]dioxol-5-yl)-1H-indazol-5-yl)amino)-5,6,7,8-tetrahydronaphthalene-2-carbonitrile;
5-((3-(1H-Indol-5-yl)-1H-indazol-5-yl)amino)-5,6,7,8-tetrahydronaphthalene-2-carbonitrile;
5-((3-(1H-Pyrrol-2-yl)-1H-indazol-5-yl)amino)-5,6,7,8-tetrahydronaphthalene-2-carbonitrile;
tert-Butyl 2-(5-((6-cyano-1,2,3,4-tetrahydronaphthalen-1-yl)amino)-1H-indazol-3-yl)-1H-pyrrole-1-carboxylate;
5-((3-(4-(Morpholine-4-carbonyl)phenyl)-1H-indazol-5-yl)amino)-5,6,7,8-tetrahydronaphthalene-2-carbonitrile;
5-((3-(2-Oxoindolin-6-yl)-1H-indazol-5-yl)amino)-5,6,7,8-tetrahydronaphthalene-2-carbonitrile;
5-((2′-Methyl-1H,2′H-[3,5′-biindazol]-5-yl)amino)-5,6,7,8-tetrahydronaphthalene-2-carbonitrile;
5-((3-(2,3-Dihydrobenzo[b][1,4]dioxin-6-yl)-1H-indazol-5-yl)amino)-5,6,7,8-tetrahydronaphthalene-2-carbonitrile;
N-(3-(5-((6-Cyano-1,2,3,4-tetrahydronaphthalen-1-yl)amino)-1H-indazol-3-yl)phenyl)acetamide;
3-(5-((6-Cyano-1,2,3,4-tetrahydronaphthalen-1-yl)amino)-1H-indazol-3-yl)-N,N-dimethylbenzamide;
5-((3-(1,4-Dioxaspiro[4.5]dec-7-en-8-yl)-1H-indazol-5-yl)amino)-5,6,7,8-tetrahydronaphthalene-2-carbonitrile;
5-((3-(3,6-Dihydro-2H-pyran-4-yl)-1H-indazol-5-yl)amino)-5,6,7,8-tetrahydronaphthalene-2-carbonitrile;
5-((3-(3-Cyanophenyl)-1H-indazol-5-yl)amino)-5,6,7,8-tetrahydronaphthalene-2-carbonitrile;
5-((3-(2-Methylpyridin-4-yl)-1H-indazol-5-yl)amino)-5,6,7,8-tetrahydronaphthalene-2-carbonitrile;
5-(5-((6-Cyano-1,2,3,4-tetrahydronaphthalen-1-yl)amino)-1H-indazol-3-yl)picolinonitrile;
5-((3-(4-Methoxyphenyl)-1H-indazol-5-yl)amino)-5,6,7,8-tetrahydronaphthalene-2-carbonitrile;
(E)-5-((3-Styryl-1H-indazol-5-yl)amino)-5,6,7,8-tetrahydronaphthalene-2-carbonitrile;
5-((3-(1-Methyl-1,2,3,6-tetrahydropyridin-4-yl)-1H-indazol-5-yl)amino)-5,6,7,8-tetrahydronaphthalene-2-carbonitrile;
5-((3-(4-Bromophenyl)-1H-indazol-5-yl)amino)-5,6,7,8-tetrahydronaphthalene-2-carbonitrile;
5-((3-(5,6-Dihydro-4H-pyrrolo[1,2-b]pyrazol-3-yl)-1H-indazol-5-yl)amino)-5,6,7,8-tetrahydronaphthalene-2-carbonitrile;
5-((3-(1-methyl-2-oxo-1,2-dihydropyridin-4-yl)-1H-indazol-5-yl)amino)-5,6,7,8-tetrahydronaphthalene-2-carbonitrile;
5-((3-(Pyridin-3-yl)-1H-indazol-5-yl)amino)-5,6,7,8-tetrahydronaphthalene-2-carbonitrile;
(Z)-5-((3-(Prop-1-en-1-yl)-1H-indazol-5-yl)amino)-5,6,7,8-tetrahydronaphthalene-2-carbonitrile;
5-((3-(5-Methylpyridin-3-yl)-1H-indazol-5-yl)amino)-5,6,7,8-tetrahydronaphthalene-2-carbonitrile;
1-Methoxy-5-{[3-(1,3-oxazol-5-yl)-1H-indazol-5-yl]amino}-5,6,7,8-tetrahydronaphthalene-2-carbonitrile;
2-Methoxy-8-((3-(oxazol-5-yl)-1H-indazol-5-yl)amino)-5,6,7,8-tetrahydroquinoline-3-carbonitrile;
7-((3-(Oxazol-5-yl)-1H-indazol-5-yl)oxy)-6,7-dihydro-5H-cyclopenta[b]pyridine-3-carbonitrile;
5-((3-(Oxazol-5-yl)-1H-indazol-5-yl)amino)-6,7,8,9-tetrahydro-5H-benzo[7]annulene-2-carbonitrile;
3,3-Dimethyl-1-((3-(oxazol-5-yl)-1H-indazol-5-yl)amino)-2,3-dihydro-1H-indene-5-carbonitrile;
2-Methyl-8-((3-(oxazol-5-yl)-1H-indazol-5-yl)amino)-5,6,7,8-tetrahydroquinoline-3-carbonitrile;
4-Methyl-7-((3-(oxazol-5-yl)-1H-indazol-5-yl)oxy)-6,7-dihydro-5H-cyclopenta[b]pyridine-3-carbonitrile;
4-Methyl-7-((3-(oxazol-yl)-1H-indazol-5-yl)amino)-6,7-dihydro-5H-cyclopenta[b]pyridine-3-carbonitrile;
7-((3-(Oxazol-5-yl)-1H-indazol-5-yl)amino)-6,7-dihydro-5H-cyclopenta[b]pyridine-3-carbonitrile;
3-Methyl-5-((3-(oxazol-5-yl)-1H-indazol-5-yl)amino)-5,6,7,8-tetrahydronaphthalene-2-carbonitrile;
1-(Methyl(3-(oxazol-5-yl)-1H-indazol-5-yl)amino)-2,3-dihydro-1H-indene-5-carbonitrile;
2-Chloro-8-((3-(oxazol-5-yl)-1H-indazol-5-yl)amino)-5,6,7,8-tetrahydroquinoline-3-carbonitrile;
4-Methoxy-1-((3-(oxazol-5-yl)-1H-indazol-5-yl)amino)-2,3-dihydro-1H-indene-5-carbonitrile;
5-((3-(Isoxazol-4-yl)-1H-indazol-5-yl)amino)-3-methoxy-5,6,7,8-tetrahydronaphthalene-2-carbonitrile;
8,8-Dimethyl-5-((3-(oxazol-5-yl)-1H-indazol-5-yl)amino)-5,6,7,8-tetrahydronaphthalene-2-carbonitrile;
5-((3-(2-Ethylpyridin-4-yl)-1H-indazol-5-yl)amino)-5,6,7,8-tetrahydronaphthalene-2-carbonitrile;
5-((3-(2-Methoxypyridin-4-yl)-1H-indazol-5-yl)amino)-5,6,7,8-tetrahydronaphthalene-2-carbonitrile;
1-((3-(Isoxazol-4-yl)-1H-indazol-5-yl)amino)-4-methyl-2,3-dihydro-1H-indene-5-carbonitrile;
5-((3-(Oxazol-5-yl)-1H-indazol-5-yl)amino)-5,6,7,8-tetrahydronaphthalene-2-carbonitrile;
8-((3-(Oxazol-5-yl)-1H-indazol-5-yl)amino)-5,6,7,8-tetrahydroquinoline-3-carbonitrile;
8-((3-(2-Ethylpyridin-4-yl)-1H-indazol-5-yl)oxy)-5,6,7,8-tetrahydroquinoline-3-carbonitrile;
8-((3-(Oxazol-5-yl)-1H-indazol-5-yl)oxy)-5,6,7,8-tetrahydroisoquinoline-3-carbonitrile;
8-((3-(2-Methoxypyridin-4-yl)-1H-indazol-5-yl)oxy)-5,6,7,8-tetrahydroquinoline-3-carbonitrile;
1-((3-Cyclopropyl-1H-indazol-5-yl)oxy)-2,3-dihydro-1H-indene-5-carbonitrile;
8-((3-Bromo-1H-indazol-5-yl)amino)-5,6,7,8-tetrahydroquinoline-3-carbonitrile;
8-((3-Chloro-1H-indazol-5-yl)amino)-5,6,7,8-tetrahydroquinoline-3-carbonitrile;
1-((3-Iodo-1H-indazol-5-yl)amino)-4-methyl-2,3-dihydro-1H-indene-5-carbonitrile;
1-((3-Iodo-1H-indazol-5-yl)amino)-2,3-dihydro-1H-indene-5-carbonitrile;
1-((3-Methyl-1H-indazol-5-yl)amino)-2,3-dihydro-1H-indene-4-carbonitrile;
3-((3-Methyl-1H-indazol-5-yl)amino)-2,3-dihydro-1H-indene-5-carbonitrile;
7-Methyl-1-((3-(oxazol-5-yl)-1H-indazol-5-yl)amino)-2,3-dihydro-1H-indene-5-carbonitrile;
5-((5-Cyano-7-fluoro-2,3-dihydro-1H-inden-1-yl)amino)-3-(oxazol-5-yl)-1H-indazol-2-ium;
5-((3-Cyclopropyl-1H-indazol-5-yl)oxy)-5,6,7,8-tetrahydroquinoline-2-carbonitrile;
1-((3-Cyclopropyl-1H-indazol-5-yl)amino)-2,3-dihydro-1H-indene-5-carbonitrile;
5-((3-Iodo-1H-indazol-5-yl)oxy)-5,6,7,8-tetrahydroquinoline-2-carbonitrile;
8-((3-Cyclopropyl-1H-indazol-5-yl)oxy)-5,6,7,8-tetrahydroquinoline-3-carbonitrile;
8-((3-Iodo-1H-indazol-5-yl)oxy)-5,6,7,8-tetrahydroquinoline-3-carbonitrile;
3,3-Difluoro-1-((3-(oxazol-5-yl)-1H-indazol-5-yl)amino)-2,3-dihydro-1H-indene-5-carbonitrile;
5-((3-(Cyclopropylmethyl)-1H-indazol-5-yl)amino)-5,6,7,8-tetrahydronaphthalene-2-carbonitrile;
3′-((3-(Oxazol-5-yl)-1H-indazol-5-yl)amino)-2′,3′-dihydrospiro[cyclopropane-1,1′-indene]-6′-carbonitrile;
8-((3-(1-(Difluoromethyl)-1H-pyrazol-4-yl)-1H-indazol-5-yl)oxy)-5,6,7,8-tetrahydroquinoline-3-carbonitrile;
8-((3-(Thiazol-5-yl)-1H-indazol-5-yl)amino)-5,6,7,8-tetrahydroquinoline-3-carbonitrile;
1-Chloro-5-((3-(oxazol-5-yl)-1H-indazol-5-yl)amino)-5,6,7,8-tetrahydronaphthalene-2-carbonitrile;
4-Chloro-8-((3-(oxazol-5-yl)-1H-indazol-5-yl)amino)-5,6,7,8-tetrahydroquinoline-3-carbonitrile;
8-((3-(Oxazol-5-yl)-1H-indazol-5-yl)amino)-4-(trifluoromethyl)-5,6,7,8-tetrahydroquinoline-3-carbonitrile;
4-Methoxy-8-((3-(oxazol-5-yl)-1H-indazol-5-yl)amino)-5,6,7,8-tetrahydroquinoline-3-carbonitrile;
4-(Difluoromethyl)-8-((3-(oxazol-5-yl)-1H-indazol-5-yl)amino)-5,6,7,8-tetrahydroquinoline-3-carbonitrile;
8-((3-(1H-Pyrrol-2-yl)-1H-indazol-5-yl)amino)-5,6,7,8-tetrahydroquinoline-3-carbonitrile;
8-((3-(1-Methyl-1H-pyrrol-3-yl)-1H-indazol-5-yl)amino)-5,6,7,8-tetrahydroquinoline-3-carbonitrile;
4-Methyl-8-((3-(oxazol-5-yl)-1H-indazol-5-yl)amino)-5,6,7,8-tetrahydroquinoline-3-carbonitrile;
2,2,4-Trifluoro-1-((3-(oxazol-5-yl)-1H-indazol-5-yl)amino)-2,3-dihydro-1H-indene-5-carbonitrile;
5-((3-(Trifluoromethyl)-1H-indazol-5-yl)oxy)-5,6,7,8-tetrahydronaphthalene-2-carbonitrile;
5-((3-(Trifluoromethyl)-1H-indazol-5-yl)amino)-5,6,7,8-tetrahydronaphthalene-2-carbonitrile;
4-Methoxy-7-((3-(oxazol-5-yl)-1H-indazol-5-yl)amino)-6,7-dihydro-5H-cyclopenta[b]pyridine-3-carbonitrile;
2,2-Difluoro-1-((3-(2-methyloxazol-5-yl)-1H-indazol-5-yl)amino)-2,3-dihydro-1H-indene-5-carbonitrile;
4-Fluoro-1-((3-(2-methyloxazol-5-yl)-1H-indazol-5-yl)oxy)-2,3-dihydro-1H-indene-5-carbonitrile;
5-((3-(1H-Pyrazol-5-yl)-1H-indazol-5-yl)amino)-5,6,7,8-tetrahydronaphthalene-2-carbonitrile;
8-((3-(3-Methylisothiazol-5-yl)-1H-indazol-5-yl)amino)-5,6,7,8-tetrahydroquinoline-3-carbonitrile;
2-Methyl-8-((3-(2-methyloxazol-5-yl)-1H-indazol-5-yl)amino)-5,6,7,8-tetrahydroquinoline-3-carbonitrile 6-Fluoro-1-((3-(2-methyloxazol-5-yl)-1H-indazol-5-yl)oxy)-2,3-dihydro-1H-indene-5-carbonitrile;
8-((3-(Pyrazin-2-yl)-1H-indazol-5-yl)oxy)-5,6,7,8-tetrahydroquinoline-3-carbonitrile;
8-Deuterio-8-((3-(oxazol-5-yl)-1H-indazol-5-yl)amino)-5,6,7,8-tetrahydroquinoline-3-carbonitrile;
5-[[3-(1,3-Oxazol-5-yl)-1H-indazol-5-yl]oxy]-5,6,7,8-tetrahydroquinoxaline-2-carbonitrile;
2,4-Dimethyl-8-[[3-(1,3-oxazol-5-yl)-1H-indazol-5-yl]amino]-5,6,7,8-tetrahydroquinoline-3-carbonitrile;
2-Methoxy-4-methyl-8-[[3-(1,3-oxazol-5-yl)-1H-indazol-5-yl]amino]-5,6,7,8-tetrahydroquinoline-3-carbonitrile;
2-Chloro-4-methyl-7-[[3-(1,3-oxazol-5-yl)-1H-indazol-5-yl]oxy]-6,7-dihydro-5H-cyclopenta[b]pyridine-3-carbonitrile;
4,6-Difluoro-1-[[3-(1,3-oxazol-5-yl)-1H-indazol-5-yl]oxy]-2,3-dihydro-1H-indene-5-carbonitrile;
trans-3-Methyl-1-[[3-(1,3-oxazol-5-yl)-1H-indazol-5-yl]amino]-2,3-dihydro-1H-indene-5-carbonitrile;
cis-3-Methyl-1-[[3-(1,3-oxazol-5-yl)-1H-indazol-5-yl]amino]-2,3-dihydro-1H-indene-5-carbonitrile;
2,4-Dimethyl-7-[[3-(1,3-oxazol-5-yl)-1H-indazol-5-yl]amino]-6,7-dihydro-5H-cyclopenta[b]pyridine-3-carbonitrile;
2-Chloro-7-[[3-(1,3-oxazol-5-yl)-1H-indazol-5-yl]oxy]-6,7-dihydro-5H-cyclopenta[b]pyridine-3-carbonitrile;
6,6-Dimethyl-7-[[3-(1,3-oxazol-5-yl)-1H-indazol-5-yl]amino]-5,7-dihydrocyclopenta[b]pyridine-3-carbonitrile;
cis-6-Methyl-5-((3-(oxazol-5-yl)-1H-indazol-5-yl)amino)-5,6,7,8-tetrahydronaphthalene-2-carbonitrile;
trans-6-Methyl-5-((3-(oxazol-5-yl)-1H-indazol-5-yl)amino)-5,6,7,8-tetrahydronaphthalene-2-carbonitrile;
cis-6-Methyl-5-[[3-(1,3-oxazol-5-yl)-1H-indazol-5-yl]oxy]-5,6,7,8-tetrahydronaphthalene-2-carbonitrile;
trans-6-Methyl-5-[[3-(1,3-oxazol-5-yl)-1H-indazol-5-yl]oxy]-5,6,7,8-tetrahydronaphthalene-2-carbonitrile;
2-Chloro-8-((3-(oxazol-5-yl)-1H-indazol-5-yl)oxy)-5,6,7,8-tetrahydroquinoline-3-carbonitrile;
2-Chloro-8-[[3-(1,2-oxazol-4-yl)-1H-indazol-5-yl]oxy]-5,6,7,8-tetrahydroquinoline-3-carbonitrile;
2-Methoxy-8-[[3-(1,3-oxazol-5-yl)-1H-indazol-5-yl]oxy]-5,6,7,8-tetrahydroquinoline-3-carbonitrile;
3-Fluoro-5-[[3-(1,3-oxazol-5-yl)-1H-indazol-5-yl]oxy]-5,6,7,8-tetrahydronaphthalene-2-carbonitrile;
2-Chloro-8-[(3-cyclopropyl-1H-indazol-5-yl)oxy]-5,6,7,8-tetrahydroquinoline-3-carbonitrile;
5-((3-(Oxazol-5-yl)-1H-indazol-5-yl)oxy)-6,7-dihydro-5H-cyclopenta[b]pyrazine-2-carbonitrile;
2-Chloro-8-[(3-cyclopropyl-1H-indazol-5-yl)amino]-4-methyl-5,6,7,8-tetrahydroquinoline-3-carbonitrile;
2-Chloro-8-[(3-cyclopropyl-1H-indazol-5-yl)oxy]-4-methyl-5,6,7,8-tetrahydroquinoline-3-carbonitrile;
2-Chloro-7-[(3-methoxy-1H-indazol-5-yl)oxy]-6,7-dihydro-5H-cyclopenta[b]pyridine-3-carbonitrile;
2-Chloro-8-[(3-methoxy-1H-indazol-5-yl)oxy]-5,6,7,8-tetrahydroquinoline-3-carbonitrile;
2-Bromo-7-[(3-methoxy-1H-indazol-5-yl)oxy]-6,7-dihydro-5H-cyclopenta[b]pyridine-3-carbonitrile;
2-Bromo-8-[(3-methoxy-1H-indazol-5-yl)oxy]-5,6,7,8-tetrahydroquinoline-3-carbonitrile;
6-Chloro-1-[(3-methoxy-1H-indazol-5-yl)oxy]-2,3-dihydro-1H-indene-5-carbonitrile;
8-[[6-Methyl-3-(1,3-oxazol-5-yl)-1H-indazol-5-yl]amino]-5,6,7,8-tetrahydroquinoline-3-carbonitrile;
4-((3-(Oxazol-5-yl)-1H-indazol-5-yl)amino)chromane-7-carbonitrile;
4-((3-(Isoxazol-4-yl)-1H-indazol-5-yl)amino)chromane-7-carbonitrile;
4-((3-Iodo-1H-indazol-5-yl)amino)chromane-7-carbonitrile; and
4-((3-(Oxazol-5-yl)-1H-indazol-5-yl)amino)-3,4-dihydro-2H-pyrano[3,2-b]pyridine-7-carbonitrile,
or a pharmaceutically acceptable salt of any of the aforementioned.

57. A pharmaceutical composition comprising a compound of any one of claims 1-56, or a pharmaceutically acceptable salt thereof, and at least one pharmaceutically acceptable carrier.

58. A method of inhibiting LRRK2 activity, said method comprising contacting a compound of any one of claims 1-56 or a pharmaceutically acceptable salt thereof with LRRK2.

59. The method of claim 58, wherein the LRRK2 is characterized by a G2019S mutation.

60. The method of claim 58, wherein the contacting comprises administering the compound to a patient.

61. A method of treating a disease or disorder associated with elevated expression or activity of LRRK2, or a functional variant thereof, said method comprising administering to a patient in need thereof a therapeutically effective amount of a compound of any one of claims 1-56, or a pharmaceutically acceptable salt thereof.

62. The method of claim 61, wherein the LRRK2 is characterized by a G2019S mutation.

63. A method for treating a neurodegenerative disease in a patient, said method comprising: administering to the patient a therapeutically effective amount of the compound of any one of claims 1-56, or a pharmaceutically acceptable salt thereof.

64. The method of claim 63, wherein said neurodegenerative disease is selected from Parkinson's disease, Parkinson disease with dementia, Parkinson's associated risksyndrome, dementia with Lewy bodies, Lewy body variant of Alzheimer's disease, combined Parkinson's disease and Alzheimer's disease, multiple system atrophy, striatonigral degeneration, olivopontocerebellar atrophy, and Shy-Drager syndrome.

65. The method of claim 63, wherein said neurodegenerative disease is Parkinson's disease.

66. The method of claim 65, wherein the Parkinson's disease is characterized by a G2019S mutation in LRRK2.
